SEQUENCE LISTING (1 sequence)

SEQ ID NO: 1 - protein, 188 aa, Homo sapiens [sequence omitted]

(19) United States
(12) Patent Application Publication
(10) Pub. No.: US 2024/0254129 A1
(43) Pub. Date: Aug. 1, 2024

(54) HETEROCYCLIC COMPOUNDS AND USES THEREOF

(71) Applicant: Kumquat Biosciences Inc., San Diego, CA (US)

(72) Inventors: Liansheng Li, San Diego, CA (US); Xiuwen Zhu, San Diego, CA (US); Zhimin Zhu, San Diego, CA (US); Pingda Ren, San Diego, CA (US); Yuan Liu, San Diego, CA (US); Yi Liu, San Diego, CA (US)

(21) Appl. No.: 18/446,017

(22) Filed: Aug. 8, 2023

Related U.S. Application Data
(63) Continuation of application No. PCT/US2022/015874, filed on Feb. 9, 2022.
(60) Provisional application No. 63/147,712, filed on Feb. 9, 2021.
(60) Provisional application No. 63/147,713, filed on Feb. 9, 2021.
(60) Provisional application No. 63/166,224, filed on Mar. 25, 2021.
(60) Provisional application No. 63/176,866, filed on Apr. 19, 2021.
(60) Provisional application No. 63/191,910, filed on May 21, 2021.

(51) Int. Cl. C07D 487/04; A61P 35/00; C07D 401/14; C07D 403/14; C07D 413/14; C07D 417/14; C07D 471/04; C07D 471/10; C07D 487/08; C07D 487/10; C07D 491/048; C07D 519/00

(52) U.S. Cl. CPC C07D 487/04; A61P 35/00; C07D 401/14; C07D 403/14; C07D 413/14; C07D 417/14; C07D 471/04; C07D 471/10; C07D 487/08; C07D 487/10; C07D 491/048; C07D 519/00

(57) ABSTRACT

The present disclosure provides compounds and pharmaceutically acceptable salt thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of International Application No. PCT/US2022/015874, filed on Feb. 9, 2022, which claims benefit of U.S. Provisional Patent Application No. 63/147,712, filed on Feb. 9, 2021, U.S. Provisional Patent Application No. 63/191,910, filed on May 21, 2021, U.S. Provisional Patent Application No. 63/147,713, filed on Feb. 9, 2021, U.S. Provisional Patent Application No. 63/166,224, filed on Mar. 25, 2021, and U.S. Provisional Patent Application No. 63/176,866, filed on Apr. 19, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Aug. 7, 2023, is named 56690_725_301_SL.xml and is 1,913 bytes in size.

### BACKGROUND

Cancer (e.g., tumor, neoplasm, metastases) is the second leading cause of death worldwide estimated to be responsible for about 10 million deaths each year. Many types of cancers are marked with mutations in one or more proteins involved in various signaling pathways leading to unregulated growth of cancerous cells. In some cases, about 25 to 30 percent (%) of tumors are known to harbor Rat sarcoma (Ras) mutations. In particular, mutations in the Kirsten Ras oncogene (K-Ras) gene are one of the most frequent Ras mutations detected in human cancers including lung adenocarcinomas (LUADs) and pancreatic ductal adenocarcinoma (PDAC).

Although Kras is known to be an oncogenic driver, there is no clinically approved targeted therapy for Ras mutant cancers thus far. Ras proteins have long been considered to be “undruggable,” due to, in part, high affinity to their substrate Guanosine-5′-triphosphate (GTP) and/or their smooth surfaces without any obvious targeting region. Recently, a specific G12C Ras gene mutation has been identified as a druggable target. However, such therapeutic approach is still limiting, as the G12C mutation in Ras has a low prevalence rate (e.g., about 3% in PDAC) as compared to other known Ras mutations including G12D, G12V, G12S mutations.

### SUMMARY

In view of the foregoing, there remains a considerable need for a new design of therapeutics and diagnostics that can specifically target Ras mutants and/or associated proteins of Ras to reduce Ras pathway signaling. Such compositions and methods can be particularly useful for treating a variety of the diseases including, but not limited to, cancers and neoplasia conditions. The present disclosure addresses these needs, and provides additional advantages applicable for diagnosis, prognosis, and treatment for a wide diversity of diseases.

In an aspect is provided a compound of Formula (I-1), or a pharmaceutically acceptable salt or solvate thereof:

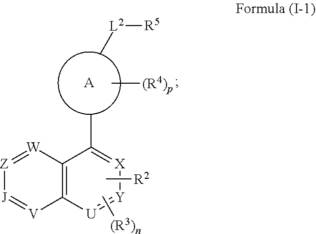

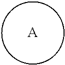

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}, or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In an aspect is provided a compound of Formula (I-2), or a pharmaceutically acceptable salt or solvate thereof:

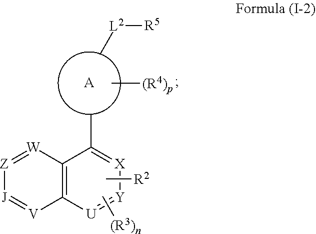

wherein:

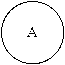

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In embodiments, p is 2, 3, 4, or 5.

In embodiments, J is C(R¹⁷).

In embodiments, R² is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}.

In embodiments, the compound, or a pharmaceutically acceptable salt or solvate thereof, has the structure of Formula (I′):

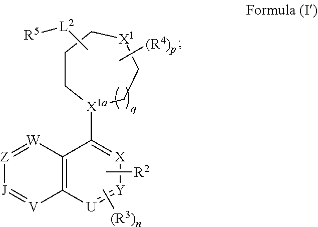

wherein X¹ is selected from C, N, O, S, S(O), and S(O)₂; X^{1a} is selected from N and C(H); and q is 1 or 2.

In embodiments, the compound, or a pharmaceutically acceptable salt or solvate thereof, has a structure selected from Formulae (Ia), (Ib), (Ic), (Id), (Ie), (If), and (Ig):

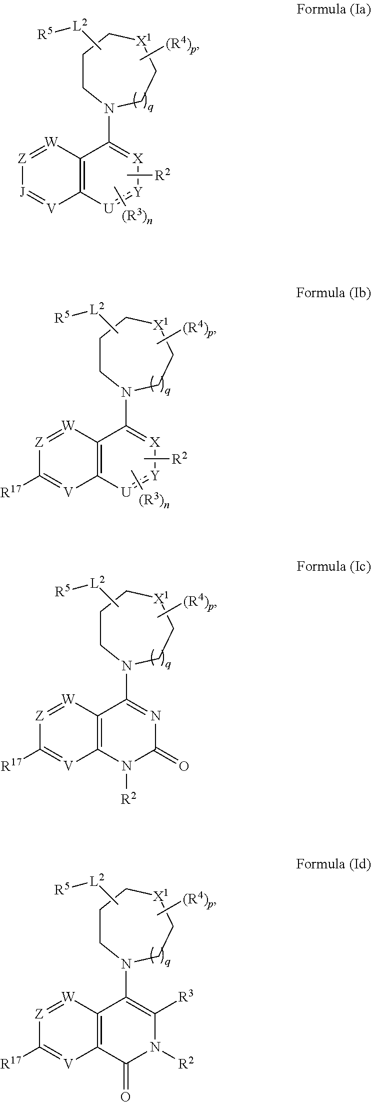

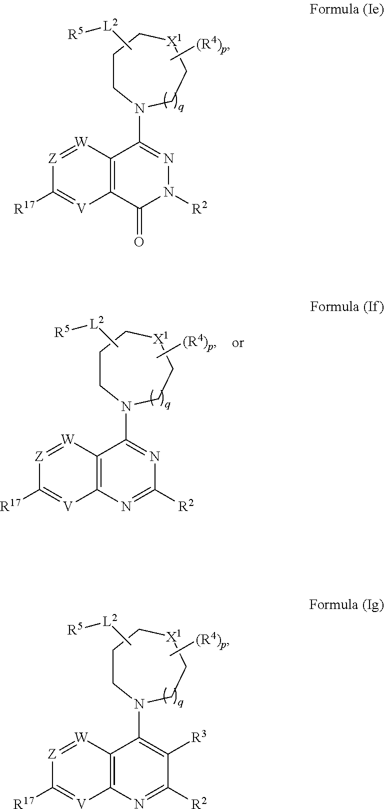

wherein X¹ is selected from C, N, O, S, S(O), and S(O)₂; and q is 1 or 2.

In an aspect is provided a compound of Formula (I″-1), or a pharmaceutically acceptable salt or solvate thereof:

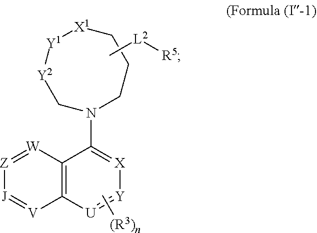

wherein:
X is C or N;
X¹ is selected from C(R+)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

In an aspect is provided a compound of Formula (I″-2), or a pharmaceutically acceptable salt or solvate thereof:

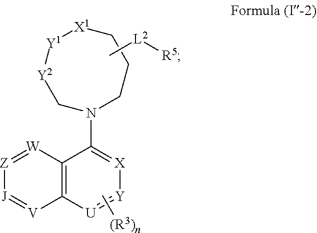

wherein:
X is C or N;
X¹ is selected from C(R⁴)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

In embodiments, Y² is a bond. In embodiments, Y² is CH₂. In embodiments, Y¹ is CH₂. In embodiments, X is N; Y is C; U is N; Z is C(R⁸); V is C(R¹⁶); J is C(R¹⁷); and W is C(R¹⁸). In embodiments, X is N; Y is C(O); U is N; Z is C(R⁸); V is N; J is C(R¹⁷); and W is C(R¹⁸). In embodiments, X is N; Y is N; U is C(O); Z is C(R⁸); V is C(R¹⁶); J is C(R¹⁷); and W is C(R¹⁸). In embodiments, X is N; Y is C; U is N; Z is N; V is N; J is C(R¹⁷); and W is C(R¹⁸). In embodiments, X is N; Y is C; U is N; Z is C(R⁸); V is C(R¹⁶); J is C(R¹⁷); and W is N. In embodiments, X is N; Y is C; U is N; Z is C(R⁸); V is N; J is C(R¹⁷); and W is C(R¹⁸).

In an aspect is provided a compound of Formula (II-1), or a pharmaceutically acceptable salt or solvate thereof:

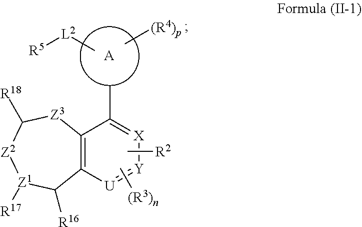

wherein:

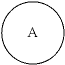

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸);
Z¹ is N or C(R⁶);
Z² is N(R⁷) or C(R⁸)(R⁹);
Z³ is absent;
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}.

R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₉alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In an aspect is provided a compound of Formula (II-2), or a pharmaceutically acceptable salt or solvate thereof:

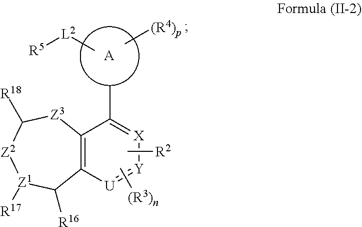

wherein:

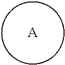

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸);
Z¹ is N or C(R⁶);
Z² is N(R⁷) or C(R⁸)(R⁹);
Z³ is absent;
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₉alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In embodiments, the compound, or a pharmaceutically acceptable salt or solvate thereof, has a structure selected from Formulae (IIa), (IIb), (IIc′), (IIc), (IId′), (IId), (IIe), (IIf), (IIg), (IIh), (IIi), (IIj), (IIk), (IIl), and (IIm):

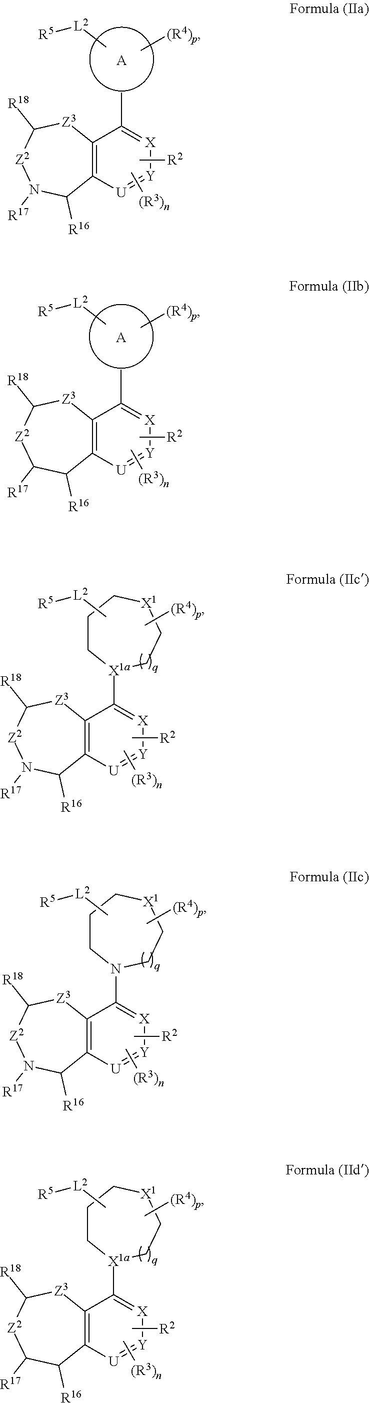

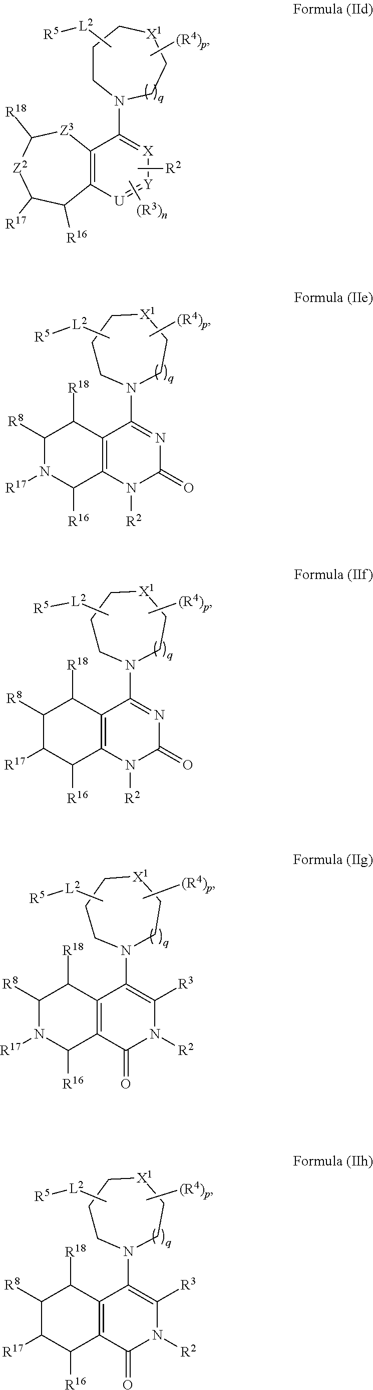

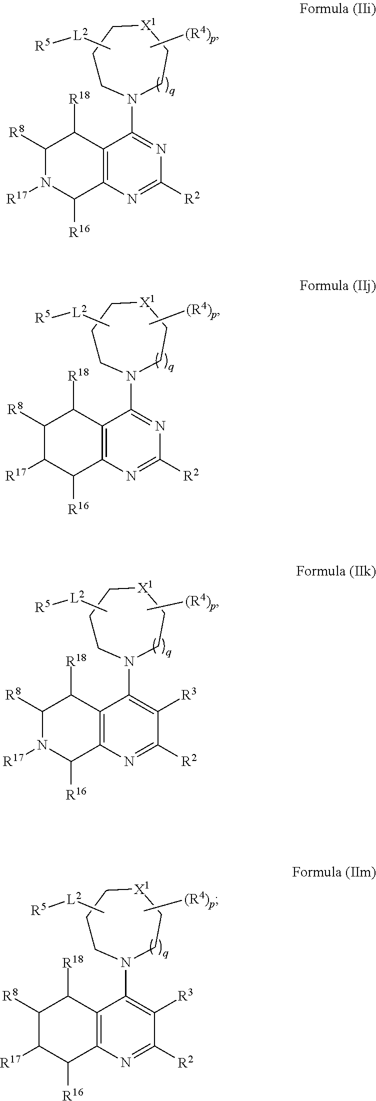

and wherein X¹ is selected from C, N, O, S, S(O), and S(O)₂; X^{1a} is selected from N and C(H); and q is 1 or 2.

In an aspect is provided a compound of Formula (IIIa-3), (IIIb-3), (IIId-3), (IIIe-3), (IIIf-3), (IIIg-3), (IIIh-3), or (IIIi-3), or a pharmaceutically acceptable salt or solvate thereof:

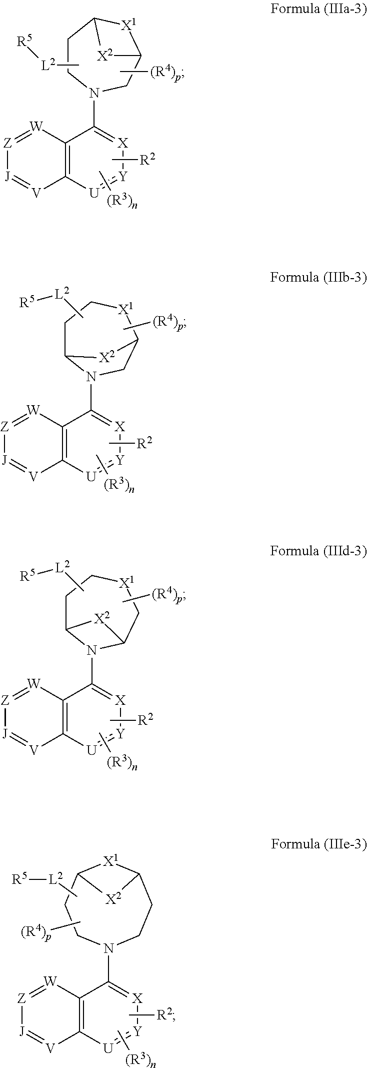

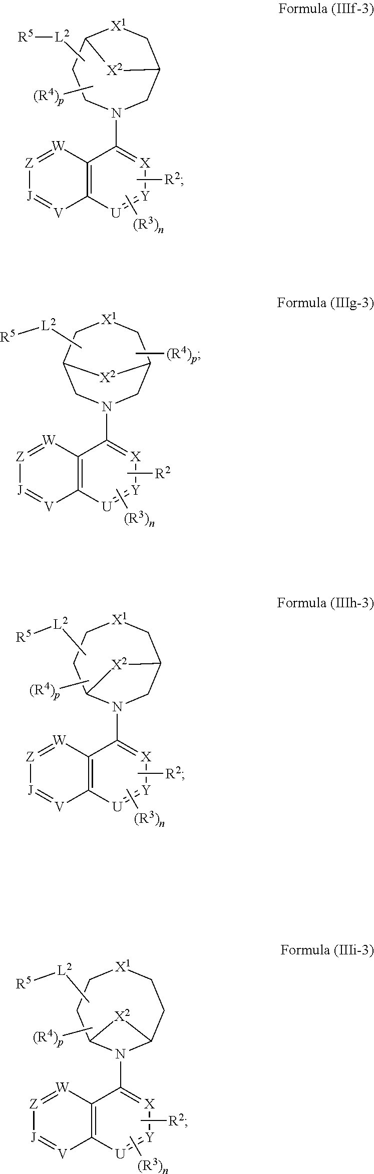

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In an aspect is provided a compound of Formula (IIIa-4), (IIIb-4), (IIId-4), (IIIe-4), (IIIf-4), (IIIg-4), (IIIh-4), or (IIIi-4), or a pharmaceutically acceptable salt or solvate thereof:

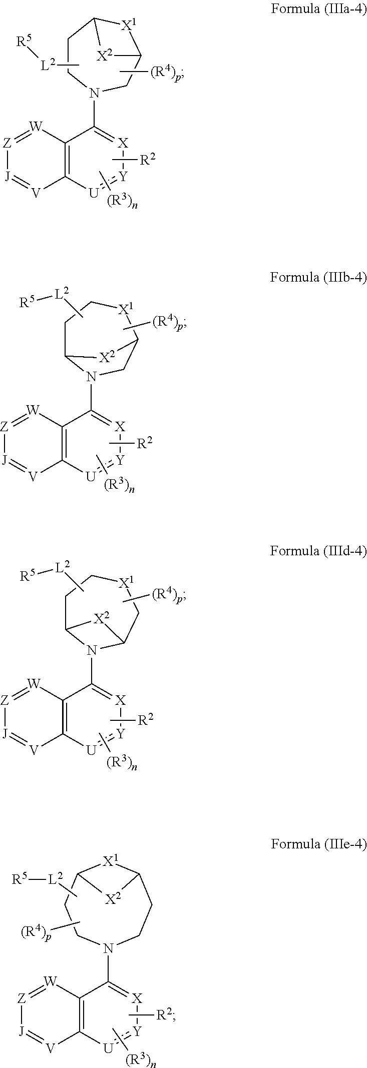

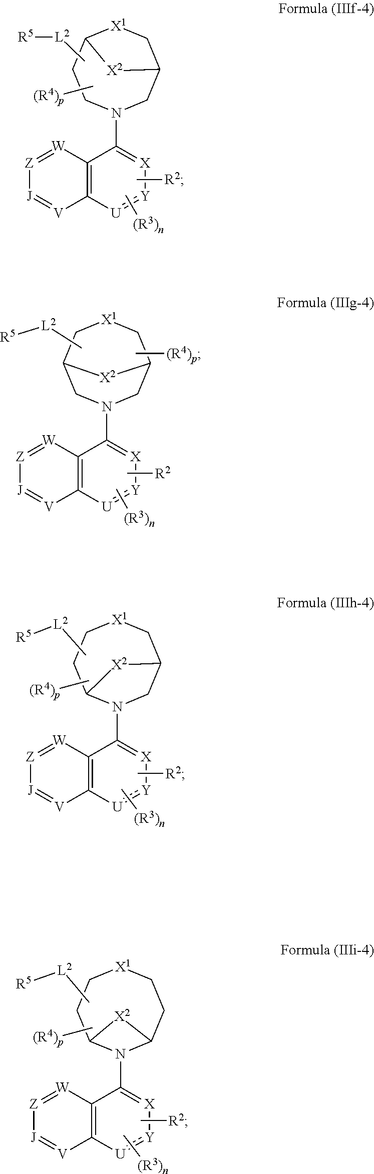

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In embodiments, the compound, or a pharmaceutically acceptable salt or solvate thereof, has the structure of Formula (IIIa-3) or (IIIa-4). In embodiments, the compound, or a pharmaceutically acceptable salt or solvate thereof, has the structure of Formula (IIIb-3) or (IIIb-4). In embodiments, the compound, or a pharmaceutically acceptable salt or solvate thereof, has the structure of Formula (IIId-3) or (IIId-4). In embodiments, the compound, or a pharmaceutically acceptable salt or solvate thereof, has the structure of Formula (IIIe-3) or (IIIe-4). In embodiments, the compound, or a pharmaceutically acceptable salt or solvate thereof, has the structure of Formula (IIIf-3) or (IIIf-4). In embodiments, the compound, or a pharmaceutically acceptable salt or solvate thereof, has the structure of Formula (IIIg-3) or (IIIg-4). In embodiments, the compound, or a pharmaceutically acceptable salt or solvate thereof, has the structure of Formula (IIIh-3) or (IIIh-4). In embodiments, the compound, or a pharmaceutically acceptable salt or solvate thereof, has the structure of Formula (IIIi-3) or (IIIi-4). In embodiments, X² is selected from —CH₂— and —CH₂CH₂—.

In an aspect is provided a compound of Formula (IVa-1), (IVb-1), or (IVc-1), or a pharmaceutically acceptable salt or solvate thereof:

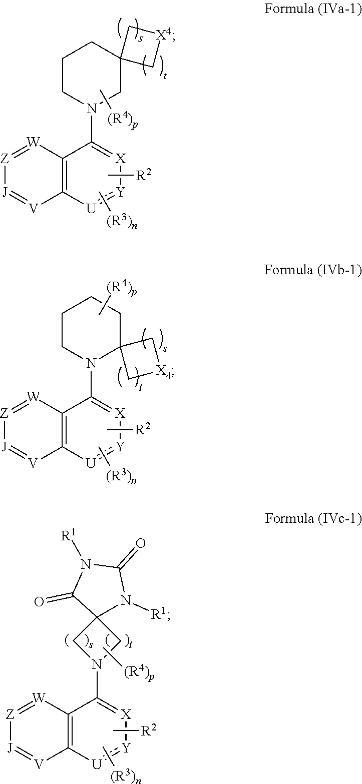

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷); W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

In an aspect is provided a compound of Formula (IVa-2), (IVb-2), or (IVc-2), or a pharmaceutically acceptable salt or solvate thereof:

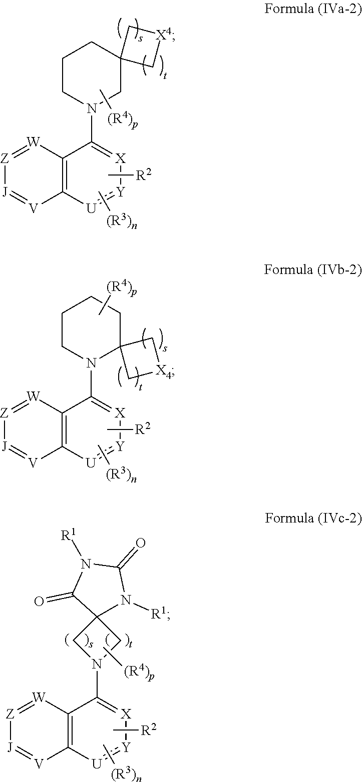

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₉haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

In embodiments, the compound, or a pharmaceutically acceptable salt or solvate thereof, has the structure of Formula (IVa-1) or (IVa-2). In embodiments, the compound, or a pharmaceutically acceptable salt or solvate thereof, has the structure of Formula (IVb-1) or (IVb-2). In embodiments, s is 1 or 2. In embodiments, t is 1 or 2. In embodiments, X⁴ is N(R¹). In embodiments, the compound, or a pharmaceutically acceptable salt or solvate thereof, has the structure of Formula (IVc-1) or (IVc-2):

In an aspect is provided a compound of Formula (Va-1), (Vb-1), or (Vc-1), or a pharmaceutically acceptable salt or solvate thereof:

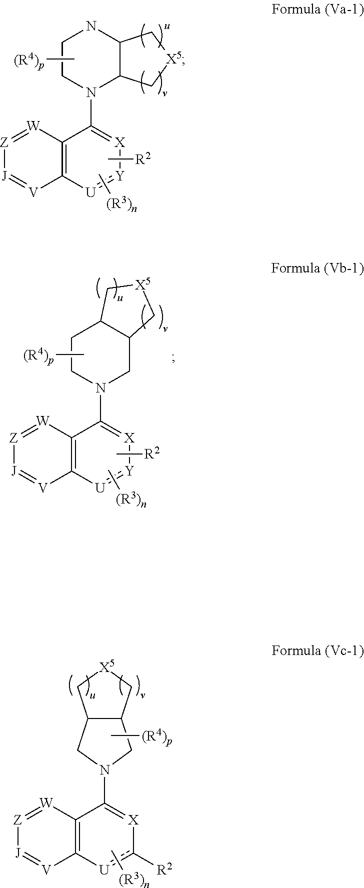

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

In an aspect is provided a compound of Formula (Va-2), (Vb-2), or (Vc-2), or a pharmaceutically acceptable salt or solvate thereof:

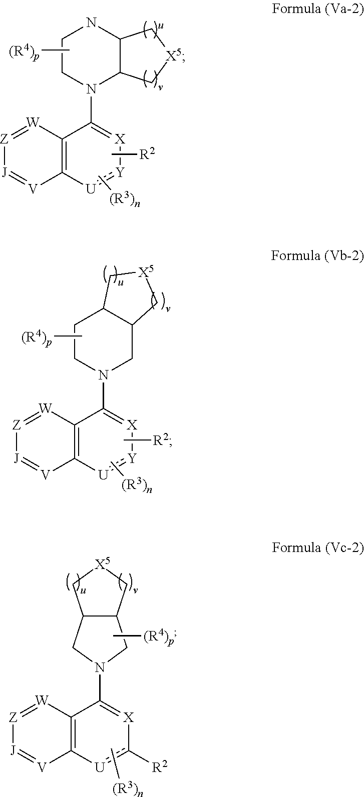

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

In embodiments, the compound, or a pharmaceutically acceptable salt or solvate thereof, has the structure of Formula (Va-1) or (Va-2). In embodiments, the compound, or a pharmaceutically acceptable salt or solvate thereof, has the structure of Formula (Vb-1) or (Vb-2). In embodiments, the compound, or a pharmaceutically acceptable salt or solvate thereof, has the structure of Formula (Vc-1) or (Vc-2). In embodiments, u is 0 or 1. In embodiments, v is 0 or 1. In embodiments, X⁵ is N(R¹). In embodiments, R¹ is hydrogen. In embodiments, R¹ is -L²-R⁵.

In an aspect is provided a compound of Formula (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof:

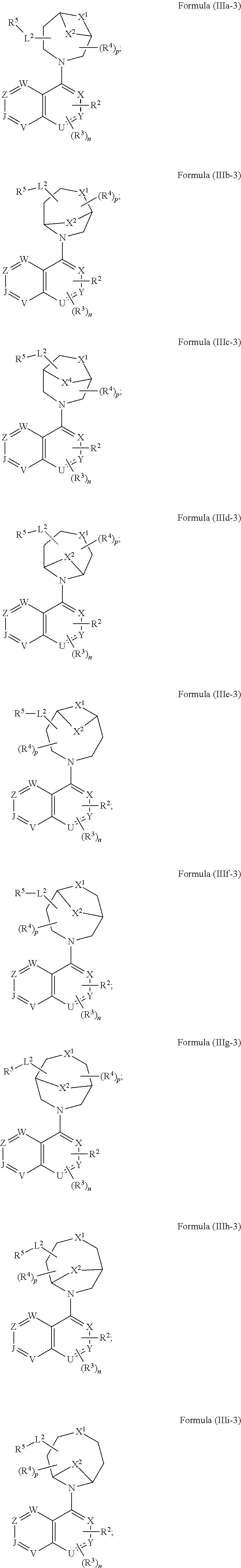

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X⁵, —CH₂—, —X⁵CH₂—, —CH₂X³—, —CH₂CH₂—, —C(H)(R⁴)—, —C(H)(R⁴)—, —X⁵—C (H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(—NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;

each R″ is independently selected from H and C₁₋₆alkyl;

each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In an aspect is provided a compound of Formula (IIIa-4), (IIIb-4), (IIIc-4), (IIId-4), (IIIc-4), (IIIf-4), (IIIg-4), (IIIh-4), or (IIIi-4), or a pharmaceutically acceptable salt or solvate thereof:

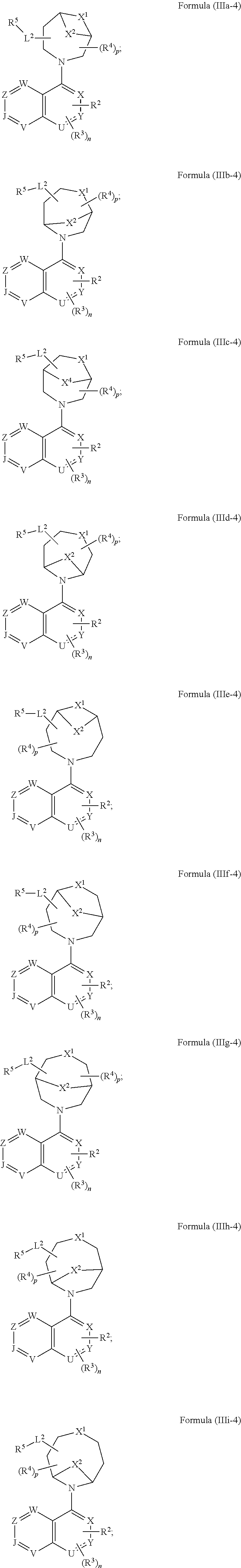

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X³, —CH₂—, —X³CH₂—, —CH₂X³—, —CH₂CH₂—, —C(H)(R⁴)—, —C(H)(R⁴)—, —X⁵—C (H)(R⁴)—, —C(H)(R⁴)X³—, —C(H)(R⁴)CH₂—, —C(H)(R⁴)C(H)(R⁴)—, —C(R⁴)₂—, —X⁵—C (R⁴)₂—, —C(R⁴)₂X⁵—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In embodiments, the compound, or a pharmaceutically acceptable salt or solvate thereof, has the structure of Formula (IIIa-3) or (IIIa-4). In embodiments, the compound, or a pharmaceutically acceptable salt or solvate thereof, has the structure of Formula (IIIb-3) or (IIIb-4). In embodiments, the compound, or a pharmaceutically acceptable salt or solvate thereof, has the structure of Formula (IIId-3) or (IIId-4). In embodiments, the compound, or a pharmaceutically acceptable salt or solvate thereof, has the structure of Formula (IIIe-3) or (IIIe-4). In embodiments, the compound, or a pharmaceutically acceptable salt or solvate thereof, has the structure of Formula (IIIf-3) or (IIIf-4). In embodiments, the compound, or a pharmaceutically acceptable salt or solvate thereof, has the structure of Formula (IIIg-3) or (IIIg-4). In embodiments, the compound, or a pharmaceutically acceptable salt or solvate thereof, has the structure of Formula (IIIh-3) or (IIIh-4). In embodiments, the compound, or a pharmaceutically acceptable salt or solvate thereof, has the structure of Formula (IIIi-3) or (IIIi-4). In embodiments, the compound, or a pharmaceutically acceptable salt or solvate thereof, has the structure of Formula (IIIc-3) or (IIIc-4).

In embodiments, X⁴ is —NH—. In embodiments, Y is C. In embodiments, Y is N. In embodiments, Y is C(O). In embodiments, X is C. In embodiments, X is N. In embodiments, U is C. In embodiments, U is N. In embodiments, U is C(O). In embodiments, Z is C(R⁸). In embodiments, R⁸ is hydrogen. In embodiments, Z is N. In embodiments, V is C(R¹⁶). In embodiments, V is C(H). In embodiments, V is N. In embodiments, J is C(R¹⁷). In embodiments, W is C(R¹⁸). In embodiments, W is C(H). In embodiments, W is N. In embodiments, R² is selected from C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In embodiments, R² is selected from —OR¹², —SR¹², and C₁₋₆alkyl, wherein C₁₋₆alkyl is optionally substituted with one, two, or three R^{20b}. In embodiments, R² is —OR¹². In embodiments, R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In embodiments, R¹² is C₁₋₆alkyl optionally substituted with one, two, or three R^{20d}. In embodiments, R¹² is C₂₋₉heterocycloalkyl optionally substituted with one, two, or three R^{20d}. In embodiments, R¹² is —CH₂—C₂₋₉heterocycloalkyl optionally substituted with one, two, or three R^{20d}.

In embodiments, each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In embodiments, each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In embodiments, R² is selected from

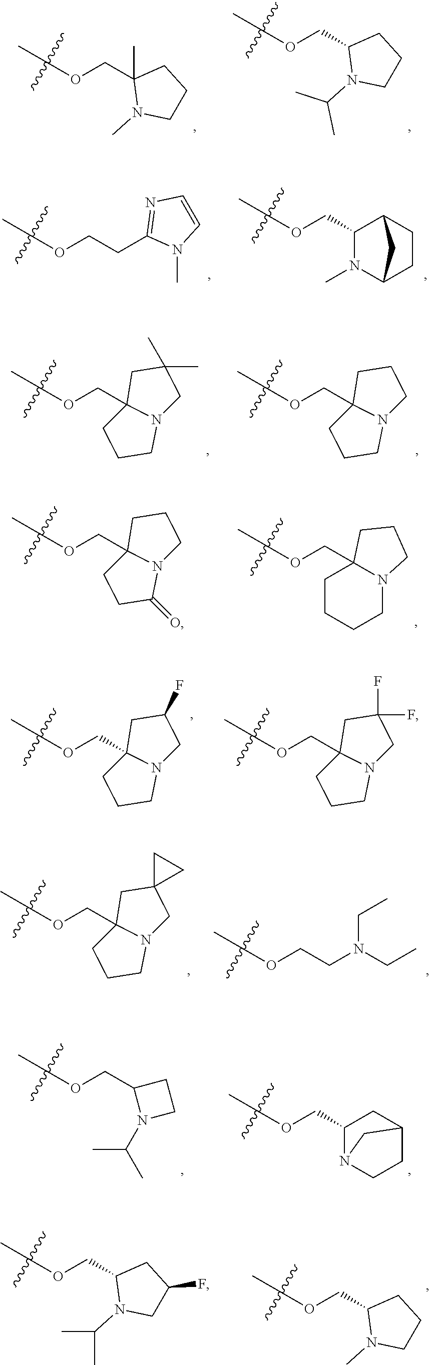

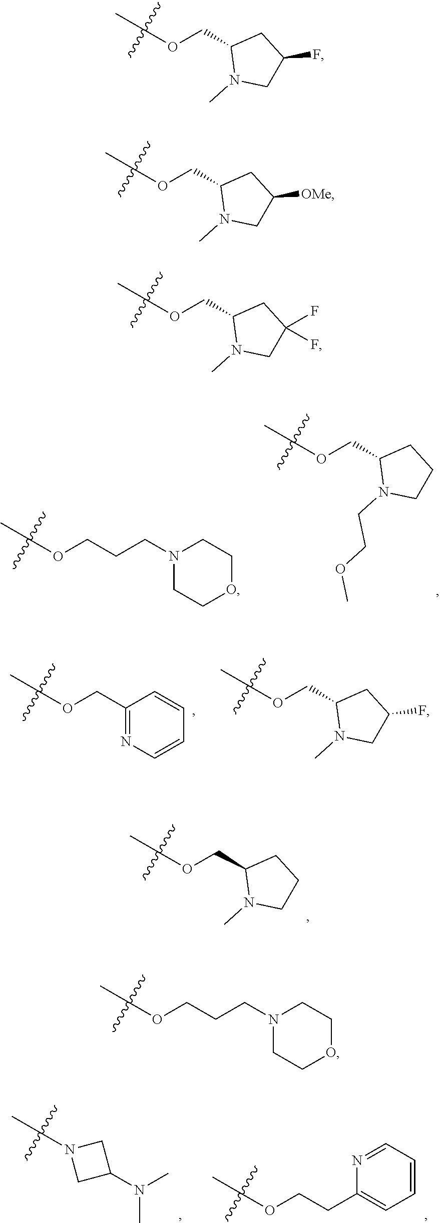

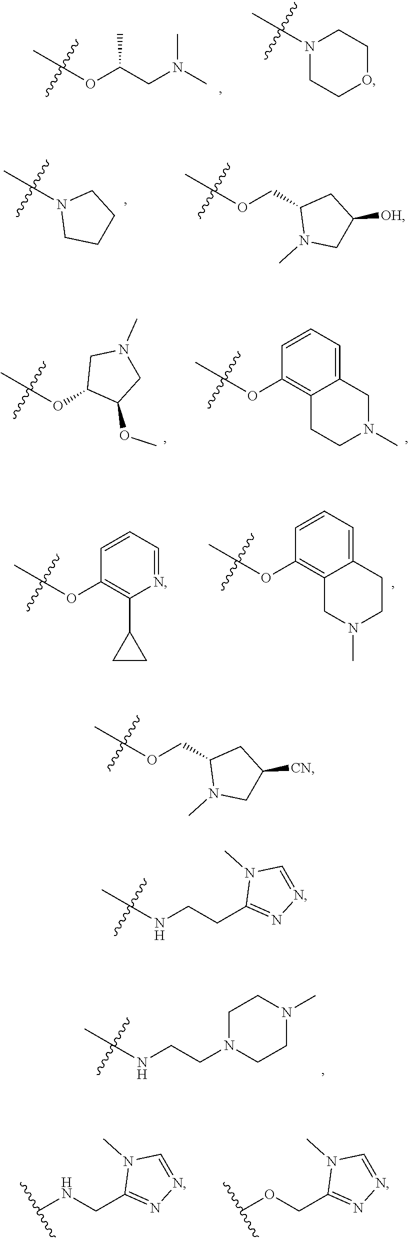

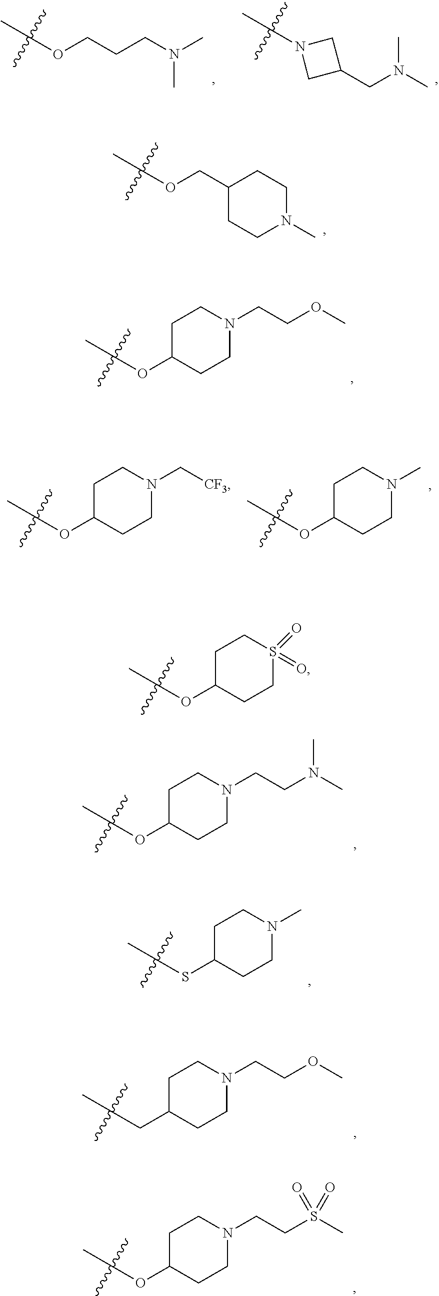

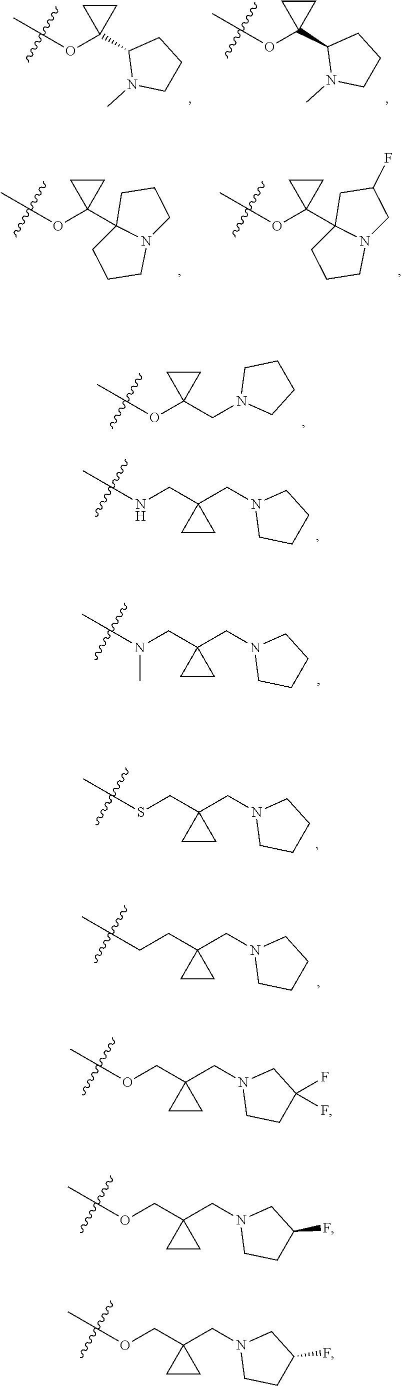

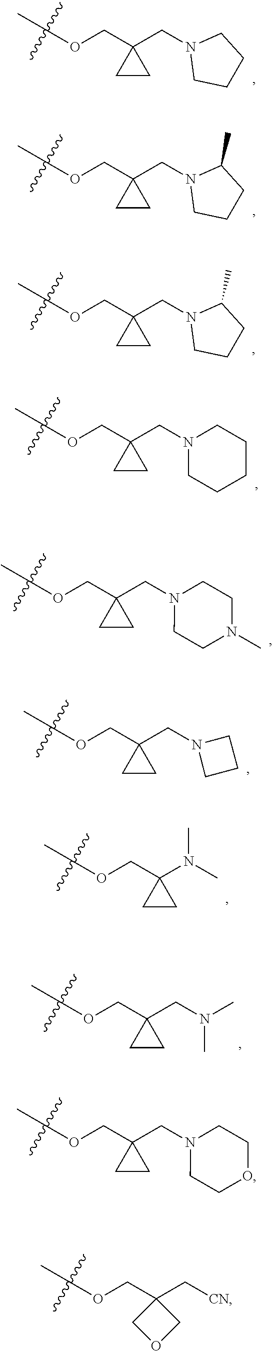

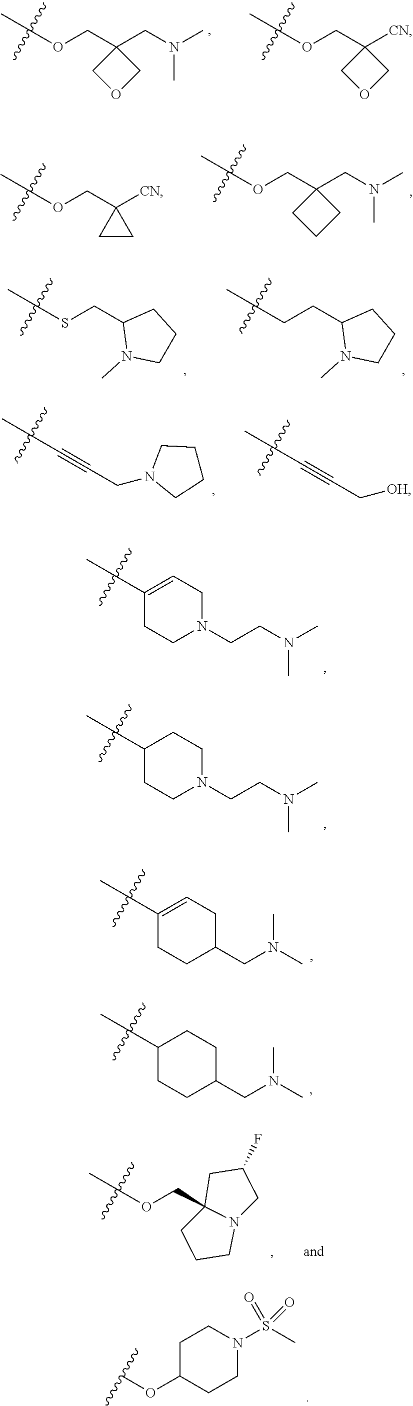

In embodiments, L¹ is a bond. In embodiments, L¹ is O. In embodiments, R¹⁹ is C₁₋₉heteroaryl optionally substituted with one, two, or three R²⁰. In embodiments, R¹⁹ is C₆₋₁₀aryl optionally substituted with one, two, or three R²⁰ⁱ. In embodiments, L² is selected from a bond, C₁-C₆alkyl, and —C(O)—. In embodiments, L² is a bond. In embodiments, L² is a C₁-C₆alkyl. In embodiments, R⁵ is hydrogen. In embodiments, R⁵ is a group other than an electrophilic moiety capable of forming a covalent bond with the cysteine residue at position 12 of a KRAS protein. In embodiments, R⁵ is an electrophilic moiety capable of forming a covalent bond with a residue at position 12 of a KRAS protein, e.g., the cysteine residue at position 12 of a KRAS protein. In embodiments. R⁵ is —H, —NH₂, —OH, —NH(C₁₋₆ alkyl),

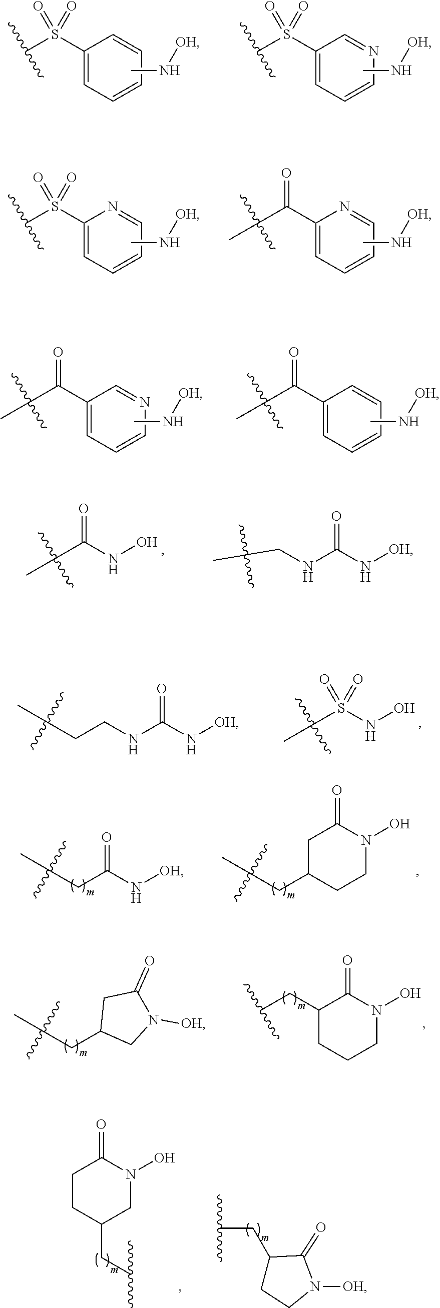

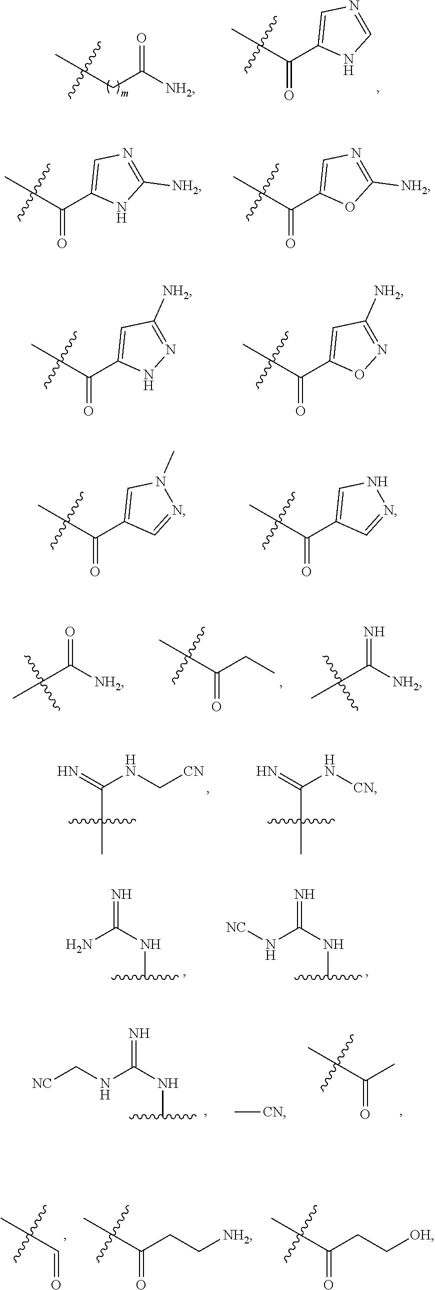

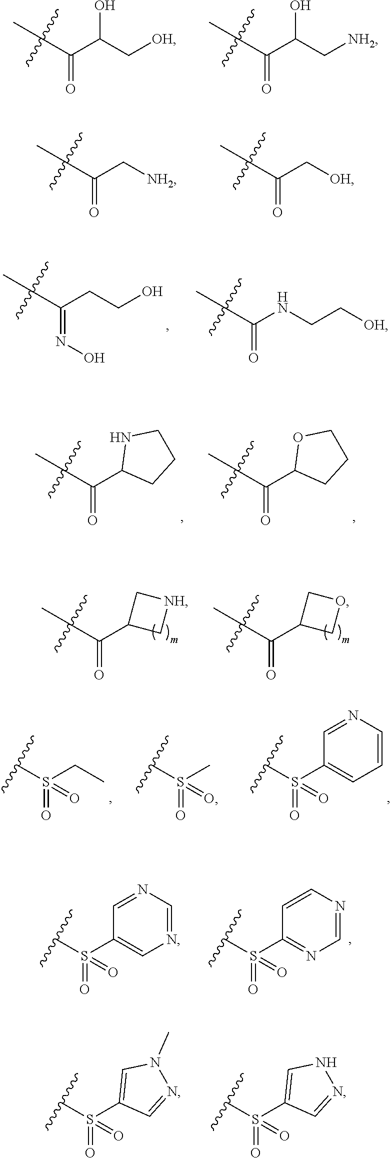

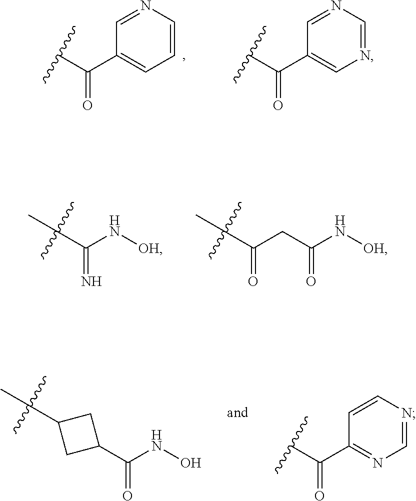 [SMILES: *C(=O)c1cccnc1 ; *C(=O)c1cncnc1 ; *C(=N)NO ; *C(=O)CC(=O)NO ; *C1CC(C(=O)NO)C1 ; *C(=O)c1ccncn1]

and m, when present, is 0, 1, 2, or 3.

In some embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently hydrogen, halogen, oxo, —CN, C₁₋₆alkyl, C₂₋₉alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₉alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently halogen. In further embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —CN. In embodiments of the subject compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —OH. In some embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa- 1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —NH₂. In further embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —C(O)OH. In select embodiments of the subject compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —OC(O)NH₂. In embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —NHC(O)NH₂. In embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —NHC(O)OH. In some embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —NHS(O)₂H. In embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —C(O)NH₂. In some embodiments of the subject compound of Formula (I-1), (I′), (I″-1), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IVa-1), (IVb-1), (IVc-1), (Va-1), (Vb-1), or (Vc-1), or a pharmaceutically acceptable salt or solvate thereof, each R⁵ is independently each R⁵ is independently hydrogen. In embodiments of the subject compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa- 1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently oxo. In further embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently C₁₋₆alkyl optionally substituted with one, two, or three R^{20a}. In select embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently C₃₋₆cycloalkyl optionally substituted with one, two, or three R^{20a}. In embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently C₂₋₉heterocycloalkyl optionally substituted with one, two, or three R^{20a}. In some embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently C₆₋₁₀aryl optionally substituted with one, two, or three R^{20a}. In further embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently each R⁵ is independently C₁₋₉heteroaryl optionally substituted with one, two, or three R^{20a}. In select embodiments of the subject compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —OR¹². In embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —N(R¹²)(R¹³). In select embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently N(R¹⁴)S(O)₂R¹⁵. In select embodiments of the subject compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, each R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In an aspect is provided a pharmaceutical composition comprising a compound described herein, or a pharmaceutically acceptable salt or solvate thereof, and a pharmaceutically acceptable excipient.

In an aspect is provided a method of treating cancer in a subject in need thereof, comprising administering to the subject a therapeutically effective amount of a subject compound disclosed herein, or a pharmaceutically acceptable salt or solvate thereof. In embodiments, the cancer is a solid tumor. In embodiments, cancer is a hematological cancer.

In an aspect is provided a method of modulating activity of a Ras protein, comprising contacting a Ras protein with an effective amount of a subject compound disclosed herein, or a pharmaceutically acceptable salt or solvate thereof, thereby modulating the activity of the Ras protein.

In embodiments, modulating comprises inhibiting the Ras protein activity. In embodiments, the Ras protein is a K-Ras protein. In embodiments, the Ras protein is a G12D, G12S, G12C, G13D, G13C, or G12V mutant K-Ras. In embodiments, the method comprises administering an additional agent or therapy. In embodiments, the additional agent or therapy is selected from the group consisting of a chemotherapeutic agent, a radioactive agent, and an immune modulator. In embodiments, modulating takes place in vitro or in vivo.

In an aspect is provided a method of inhibiting cell growth, comprising administering a cell expressing a Ras protein with an effective amount of a subject compound disclosed herein, or a pharmaceutically acceptable salt or solvate thereof, thereby inhibiting growth of said cells. In embodiments, the method comprises administering an additional agent to said cell. In embodiments, the additional agent is a chemotherapeutic agent, a radioactive agent, or an immune modulator.

In an aspect is provided a Ras protein modulated by a subject compound disclosed herein. In yet another aspect is provided a Ras protein bound by a subject compound disclosed herein, or a pharmaceutically acceptable salt or solvate thereof, wherein activity of said Ras protein is reduced as compared to a Ras protein unbound to said compound.

In one aspect, the disclosure provides a compound of Formula (I), or a pharmaceutically acceptable salt or solvate thereof:

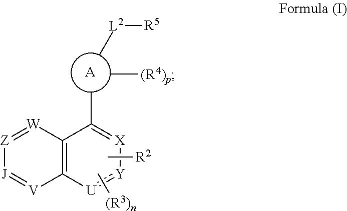

wherein:

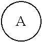

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;

R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound of Formula (I) having the structure of Formula (I′), or a pharmaceutically acceptable salt or solvate thereof:

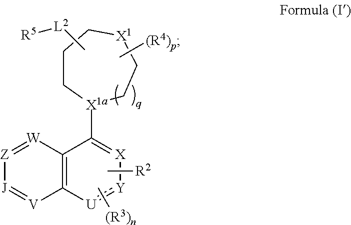

wherein X¹ is selected from C, N, O, S, S(O), and S(O)₂; X^{1a} is selected from N and C(H); and q is 1 or 2.

In some embodiments is a compound of Formula (I) having the structure of Formula (Ia), or a pharmaceutically acceptable salt or solvate thereof:

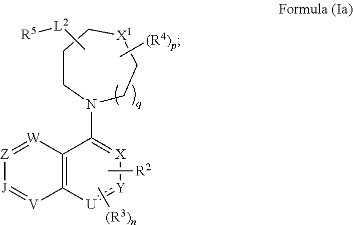

wherein X¹ is selected from C, N, O, S, S(O), and S(O)₂; and q is 1 or 2.

In some embodiments is a compound of Formula (I) having the structure of Formula (Ib), or a pharmaceutically acceptable salt or solvate thereof:

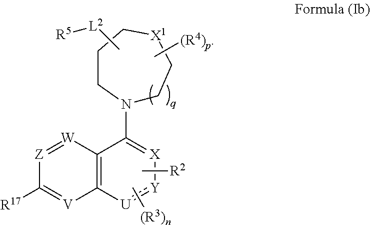

In some embodiments is a compound of Formula (I) having the structure of Formula (Ic), or a pharmaceutically acceptable salt or solvate thereof:

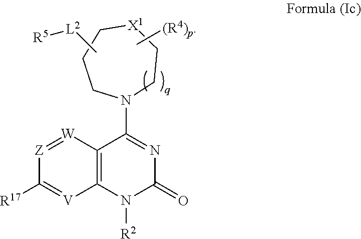

In some embodiments is a compound of Formula (I) having the structure of Formula (Id), or a pharmaceutically acceptable salt or solvate thereof:

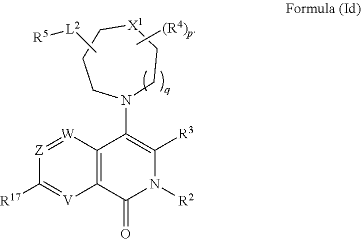

In some embodiments is a compound of Formula (I) having the structure of Formula (Ie), or a pharmaceutically acceptable salt or solvate thereof:

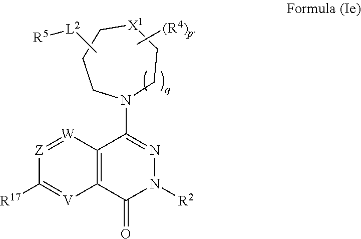

In some embodiments is a compound of Formula (I) having the structure of Formula (If), or a pharmaceutically acceptable salt or solvate thereof:

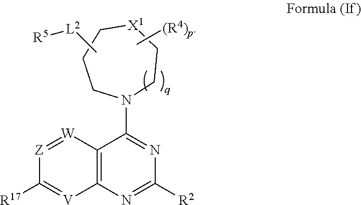

In some embodiments is a compound of Formula (I) having the structure of Formula (Ig), or a pharmaceutically acceptable salt or solvate thereof:

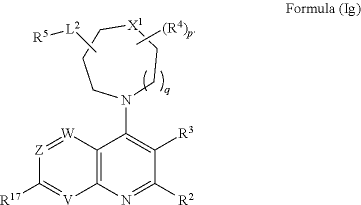

In another aspect, the disclosure provides a compound of Formula (I″), or a pharmaceutically acceptable salt or solvate thereof:

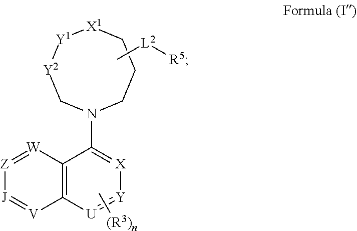

wherein:
X is C or N;
X¹ is selected from C(R⁴)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ,
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

In some embodiments is a compound of Formula (I″), or a pharmaceutically acceptable salt or solvate thereof, wherein Y² is a bond. In some embodiments is a compound of Formula (I″), or a pharmaceutically acceptable salt or solvate thereof, wherein Y² is CH₂. In some embodiments is a compound of Formula (I″), or a pharmaceutically acceptable salt or solvate thereof, wherein Y¹ is CH₂. In some embodiments is a compound of Formula (I′), (I″), (Ia), (Ib), (Ic), (Id), (Ie), (If), or (Ig), or a pharmaceutically acceptable salt or solvate thereof, wherein X¹ is N(H). In some embodiments is a compound of Formula (I′), (I″), (Ia), (Ib), (Ic), (Id), (Ie), (If), or (Ig), or a pharmaceutically acceptable salt or solvate thereof, wherein X¹ is N(R⁴). In some embodiments is a compound of Formula (I′), (I″), (Ia), (Ib), (Ic), (Id), (Ie), (If), or (Ig), or a pharmaceutically acceptable salt or solvate thereof, wherein X¹ is N(-L²-R⁵). In some embodiments is a compound of Formula (I′), (I″), (Ia), (Ib), (Ic), (Id), (Ie), (If), or (Ig), or a pharmaceutically acceptable salt or solvate thereof, wherein X¹ is O. In some embodiments is a compound of Formula (I′), (I″), (Ia), (Ib), (Ic), (Id), (Ie), (If), or (Ig), or a pharmaceutically acceptable salt or solvate thereof, wherein X¹ is S. In some embodiments is a compound of Formula (I′), (I″), (Ia), (Ib), (Ic), (Id), (Ie), (If), or (Ig), or a pharmaceutically acceptable salt or solvate thereof, wherein X¹ is S(O)₂. In some embodiments is a compound of Formula (I′), (I″), (Ia), (Ib), (Ic), (Id), (Ie), (If), or (Ig), or a pharmaceutically acceptable salt or solvate thereof, wherein X¹ is CH₂. In some embodiments is a compound of Formula (I′), (I″), (Ia), (Ib), (Ic), (Id), (Ie), (If), or (Ig), or a pharmaceutically acceptable salt or solvate thereof, wherein X¹ is C(H)(R⁴). In some embodiments is a compound of Formula (I′), (I″), (Ia), (Ib), (Ic), (Id), (Ie), (If), or (Ig), or a pharmaceutically acceptable salt or solvate thereof, wherein X¹ is C(R⁴)₂. In some embodiments is a compound of Formula (I′), (I″), (Ia), (Ib), (Ic), (Id), (Ie), (If), or (Ig), or a pharmaceutically acceptable salt or solvate thereof, wherein X¹ is C(H)(-L²-R⁵).

In some embodiments is a compound of Formula (I′), (Ia), (Ib), (Ic), (Id), (Ie), (If), or (Ig), or a pharmaceutically acceptable salt or solvate thereof, wherein q is 1. In some embodiments is a compound of Formula (I′), (Ia), (Ib), (Ic), (Id), (Ie), (If), or (Ig), or a pharmaceutically acceptable salt or solvate thereof, wherein q is 2.

In some embodiments is a compound of Formula (I), (I′), (I″), (Ia), (Ib), (Ic), (Id), (Ie), (If), or (Ig), or a pharmaceutically acceptable salt or solvate thereof, wherein V is C(R¹⁶). In some embodiments is a compound of Formula (I), (I′), (I″), (Ia), (Ib), (Ic), (Id), (Ie), (If), or (Ig), or a pharmaceutically acceptable salt or solvate thereof, wherein V is C(H). In some embodiments is a compound of Formula (I), (I′), (I″), (Ia), (Ib), (Ic), (Id), (Ie), (If), or (Ig), or a pharmaceutically acceptable salt or solvate thereof, wherein V is N.

In some embodiments is a compound of Formula (I), (I′), (I″), (Ia), (Ib), (Ic), (Id), (Ie), (If), or (Ig), or a pharmaceutically acceptable salt or solvate thereof, wherein W is C(R¹⁸). In some embodiments is a compound of Formula (I), (I′), (I″), (Ia), (Ib), (Ic), (Id), (Ie), (If), or (Ig), or a pharmaceutically acceptable salt or solvate thereof, wherein W is C(H). In some embodiments is a compound of Formula (I), (I′), (I″), (Ia), (Ib), (Ic), (Id), (Ie), (If), or (Ig), or a pharmaceutically acceptable salt or solvate thereof, wherein W is N.

In some embodiments is a compound of Formula (I), (I′), (I″), (Ia), (Ib), (Ic), (Id), (Ie), (If), or (Ig), or a pharmaceutically acceptable salt or solvate thereof, wherein Z is C(R⁸). In some embodiments is a compound of Formula (I), (I′), (I″), (Ia), (Ib), (Ic), (Id), (Ie), (If), or (Ig), or a pharmaceutically acceptable salt or solvate thereof, wherein Z is C(H). In some embodiments is a compound of Formula (I), (I′), (I″), (Ia), (Ib), (Ic), (Id), (Ie), (If), or (Ig), or a pharmaceutically acceptable salt or solvate thereof, wherein Z is N.

In another aspect, the disclosure provides a compound of Formula (II), or a pharmaceutically acceptable salt or solvate

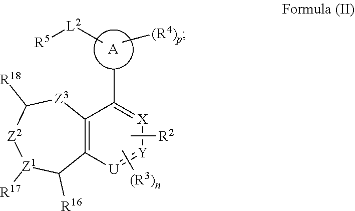

wherein:

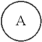

is a 7- or 8-membered monocyclic heterocycloalkyl ring;

X is C or N;
Y is C, S(O), S(O)₂, C(O), or N;
U is C, S(O), S(O)₂, C(O), or N;
W is N or C(R¹⁸);
Z¹ is N or C(R⁶);
Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}, or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};

R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, R²⁰ⁱ, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

In some embodiments is a compound of Formula (II) having the structure of Formula (IIa), or a pharmaceutically acceptable salt or solvate thereof:

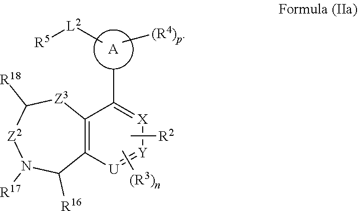

In some embodiments is a compound of Formula (II) having the structure of Formula (IIb), or a pharmaceutically acceptable salt or solvate thereof:

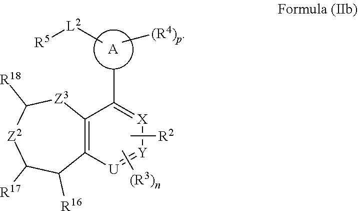

In some embodiments is a compound of Formula (II) having the structure of Formula (IIc′), or a pharmaceutically acceptable salt or solvate thereof:

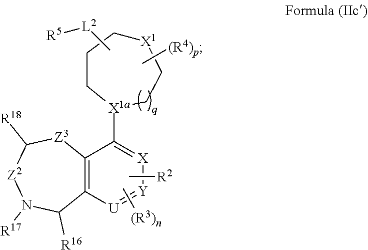

wherein X¹ is selected from C, N, O, S, S(O), and S(O)₂; X^{1a} is selected from N and C(H); and q is 1 or 2.

In some embodiments is a compound of Formula (II) having the structure of Formula (IIc), or a pharmaceutically acceptable salt or solvate thereof:

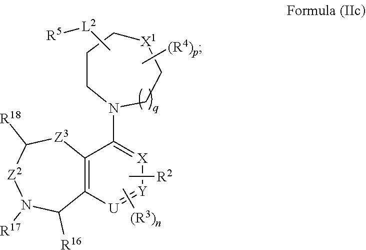

wherein X¹ is selected from C, N, O, S, S(O), and S(O)₂; and q is 1 or 2.

In some embodiments is a compound of Formula (II) having the structure of Formula (IId′), or a pharmaceutically acceptable salt or solvate thereof:

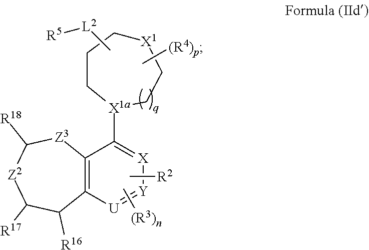

wherein X¹ is selected from C, N, O, S, S(O), and S(O)₂; X^{1a} is selected from N and C(H); and q is 1 or 2.

In some embodiments is a compound of Formula (II) having the structure of Formula (IId), or a pharmaceutically acceptable salt or solvate thereof:

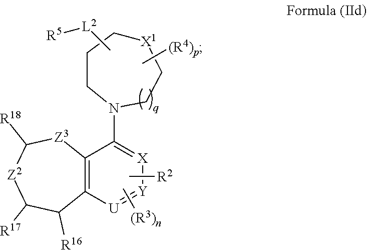

wherein X¹ is selected from C, N, O, S, S(O), and S(O)₂; and q is 1 or 2.

In some embodiments is a compound of Formula (II), (IIa), (IIb), (IIc′), (IIc), (IId′), or (IId), or a pharmaceutically acceptable salt or solvate thereof, wherein X¹ is N(H). In some embodiments is a compound of Formula (II), (IIa), (IIb), (IIc′), (IIc), (IId′), or (IId), or a pharmaceutically acceptable salt or solvate thereof, wherein X¹ is N(R⁴). In some embodiments is a compound of Formula (II), (IIa), (IIb), (IIc′), (IIc), (IId′), or (IId), or a pharmaceutically acceptable salt or solvate thereof, wherein X¹ is N(-L²-R⁵). In some embodiments is a compound of Formula (II), (IIa), (IIb), (IIc′), (IIc), (IId′), or (IId), or a pharmaceutically acceptable salt or solvate thereof, wherein X¹ is O. In some embodiments is a compound of Formula (II), (IIa), (IIb), (IIc′), (IIc), (IId′), or (IId), or a pharmaceutically acceptable salt or solvate thereof, wherein X¹ is S. In some embodiments is a compound of Formula (II), (IIa), (IIb), (IIc′), (IIc), (IId′), or (IId), or a pharmaceutically acceptable salt or solvate thereof, wherein X¹ is S(O)₂. In some embodiments is a compound of Formula (II), (IIa), (IIb), (IIc′), (IIc), (IId′), or (IId), or a pharmaceutically acceptable salt or solvate thereof, wherein X¹ is CH₂. In some embodiments is a compound of Formula (II), (IIa), (IIb), (IIc′), (IIc), (IId′), or (IId), or a pharmaceutically acceptable salt or solvate thereof, wherein X¹ is C(H)(R⁴). In some embodiments is a compound of Formula (II), (IIa), (IIb), (IIc′), (IIc), (IId′), or (IId), or a pharmaceutically acceptable salt or solvate thereof, wherein X¹ is C(R⁴)₂. In some embodiments is a compound of Formula (II), (IIa), (IIb), (IIc′), (IIc), (IId′), or (IId), or a pharmaceutically acceptable salt or solvate thereof, wherein X¹ is C(H)(-L²-R⁵).

In some embodiments is a compound of Formula (II), (IIa), (IIb), (IIc′), (IIc), (IId′), or (IId), or a pharmaceutically acceptable salt or solvate thereof, wherein q is 1. In some embodiments is a compound of Formula (II), (IIa), (IIb), (IIc′), (IIc), (IId′), or (IId), or a pharmaceutically acceptable salt or solvate thereof, wherein q is 2.

In some embodiments is a compound of Formula (II), (IIa), (IIb), (IIc′), (IIc), (IId′), or (IId), or a pharmaceutically acceptable salt or solvate thereof, wherein Z³ is absent.

In some embodiments is a compound of Formula (II), (IIa), (IIb), (IIc′), (IIc), (IId′), or (IId), or a pharmaceutically acceptable salt or solvate thereof, wherein Z² is C(R⁸)(R⁹).

In some embodiments is a compound of Formula (II), (IIa), (IIb), (IIc′), (IIc), (IId′), or (IId), or a pharmaceutically acceptable salt or solvate thereof, wherein R⁹ is hydrogen.

In some embodiments is a compound of Formula (II), (IIa), (IIb), (IIc′), (IIc), (IId′), or (IId), or a pharmaceutically acceptable salt or solvate thereof, wherein X is C. In some embodiments is a compound of Formula (II), (IIa), (IIb), (IIc′), (IIc), (IId′), or (IId), or a pharmaceutically acceptable salt or solvate thereof, wherein X is N.

In some embodiments is a compound of Formula (II), (IIa), (IIb), (IIc′), (IIc), (IId′), or (IId), or a pharmaceutically acceptable salt or solvate thereof, wherein Y is C. In some embodiments is a compound of Formula (II), (IIa), (IIb), (IIc′), (IIc), (IId′), or (IId), or a pharmaceutically acceptable salt or solvate thereof, wherein Y is N. In some embodiments is a compound of Formula (II), (IIa), (IIb), (IIc′), (IIc), (IId′), or (IId), or a pharmaceutically acceptable salt or solvate thereof, wherein Y is C(O).

In some embodiments is a compound of Formula (II), (IIa), (IIb), (IIc′), (IIc), (IId′), or (IId), or a pharmaceutically acceptable salt or solvate thereof, wherein U is C. In some embodiments is a compound of Formula (II), (IIa), (IIb), (IIc′), (IIc), (IId′), or (IId), or a pharmaceutically acceptable salt or solvate thereof, wherein U is N. In some embodiments is a compound of Formula (II), (IIa), (IIb), (IIc′), (IIc), (IId′), or (IId), or a pharmaceutically acceptable salt or solvate thereof, wherein U is C(O).

In some embodiments is a compound of Formula (II) having the structure of Formula (IIe), or a pharmaceutically acceptable salt or solvate thereof:

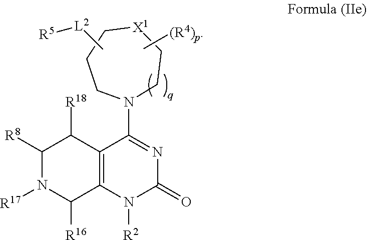

In some embodiments is a compound of Formula (II) having the structure of Formula (IIf), or a pharmaceutically acceptable salt or solvate thereof:

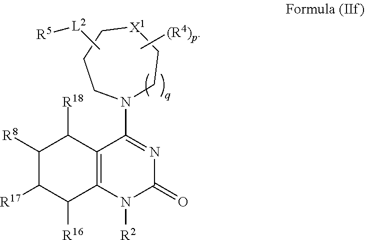

In some embodiments is a compound of Formula (II) having the structure of Formula (IIg), or a pharmaceutically acceptable salt or solvate thereof:

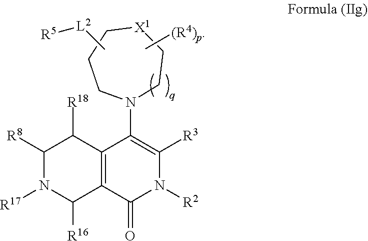

In some embodiments is a compound of Formula (II) having the structure of Formula (IIh), or a pharmaceutically acceptable salt or solvate thereof:

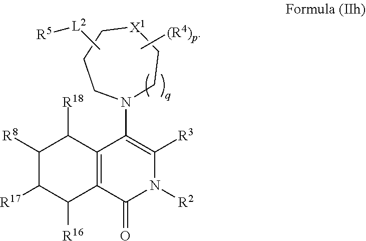

In some embodiments is a compound of Formula (II) having the structure of Formula (III), or a pharmaceutically acceptable salt or solvate thereof:

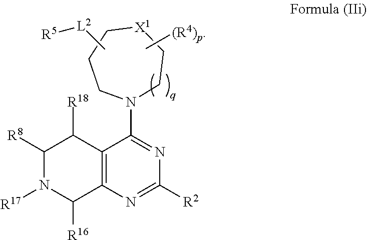

In some embodiments is a compound of Formula (II) having the structure of Formula (IIj), or a pharmaceutically acceptable salt or solvate thereof:

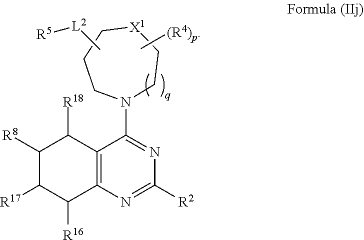

In some embodiments is a compound of Formula (II) having the structure of Formula (IIIk), or a pharmaceutically acceptable salt or solvate thereof:

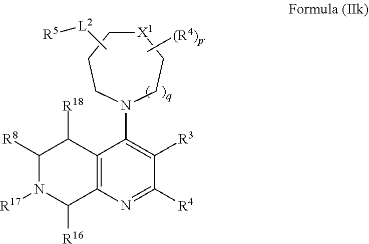

In some embodiments is a compound of Formula (II) having the structure of Formula (IIm), or a pharmaceutically acceptable salt or solvate thereof:

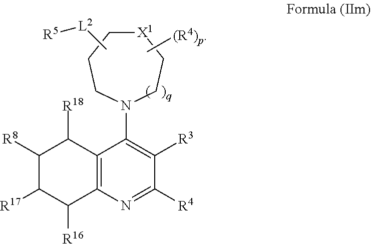

In another aspect, the disclosure provides a compound of Formula (IIIa)-(IIIi), or a pharmaceutically acceptable salt or solvate thereof:

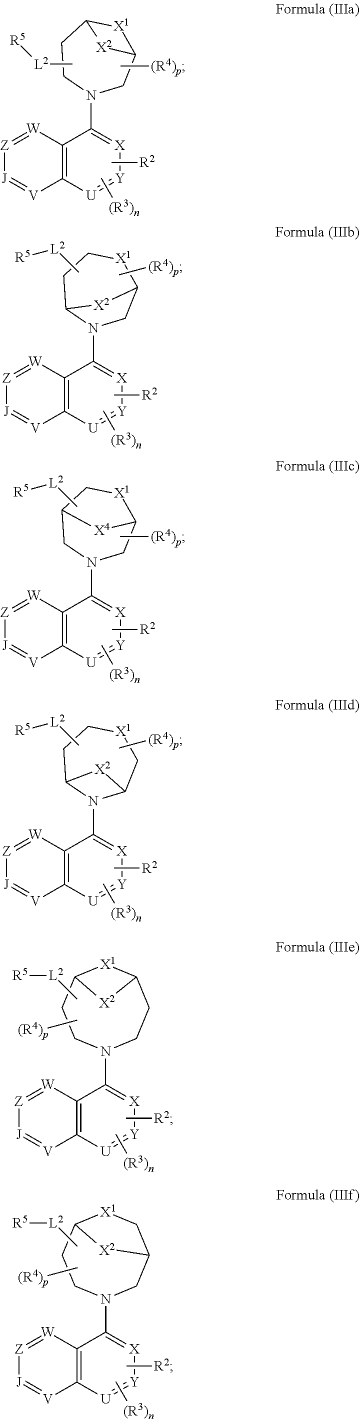

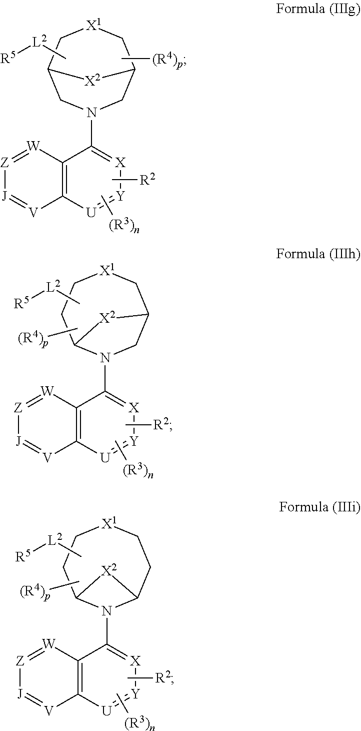

wherein:
X is C or N;
X¹ is selected from C(R⁴)(R⁶), N(R⁴), N(R⁶), O, S, S(O), and S(O)₂;
X² is selected from X³, —CH₂, —X³CH₂—, —CH₂X³—, —CH₂CH₂—, —C(H)(R+)—, —C(H)(R+)—, —X⁵C(H)(R+)—, —C(H)(R+)X³—, —C(H)(R⁴)CH₂—, —C(H)(R⁴)C(H)(R⁴), —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X⁵, —CH₂—, —X⁵CH₂—, —CH₂X⁵—, —CH₂CH₂—, —C(H)(R⁴)₂—, —C(H)(R⁴)₂—, —X⁵C(H)(R⁴)₂—, —C(H)(R⁴)X⁵—, —C(H)(R⁴)CH₂—, —C(H)(R⁴)C(H)(R⁴)—, —C(R⁴)₂—, —, —X⁵C(R⁴)₂—, —C(R⁴)₂X⁵—, —C(H)C(R⁴)₂—, —C(R⁴), C(H)—, and —C(R⁴), C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound having the structure of Formula (IIIa), or a pharmaceutically acceptable salt or solvate thereof:

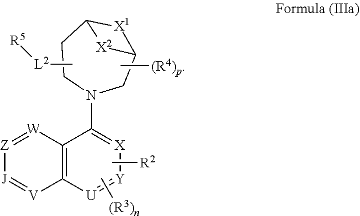

In some embodiments is a compound having the structure of Formula (IIIb), or a pharmaceutically acceptable salt or solvate thereof:

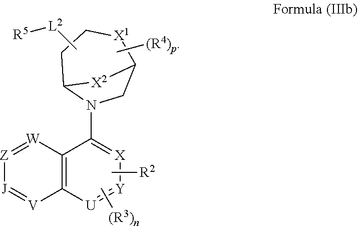

In some embodiments is a compound having the structure of Formula (IIId), or a pharmaceutically acceptable salt or solvate thereof.

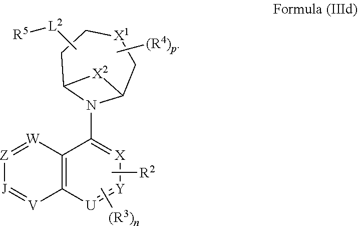

In some embodiments is a compound having the structure of Formula (IIIe), or a pharmaceutically acceptable salt or solvate thereof.

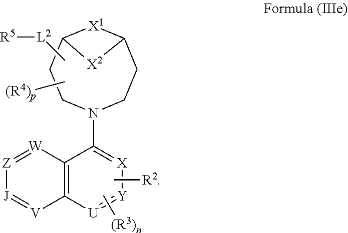

In some embodiments is a compound having the structure of Formula (IIIf), or a pharmaceutically acceptable salt or solvate thereof:

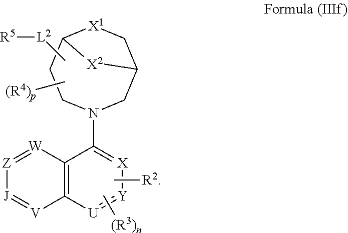

In some embodiments is a compound having the structure of Formula (IIIg), or a pharmaceutically acceptable salt or solvate thereof.

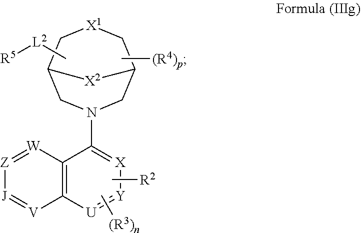

In some embodiments is a compound having the structure of Formula (IIIh), or a pharmaceutically acceptable salt or solvate thereof:

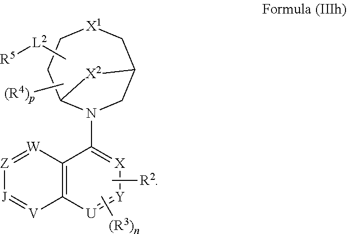

In some embodiments is a compound having the structure of Formula (IIIi), or a pharmaceutically acceptable salt or solvate thereof:

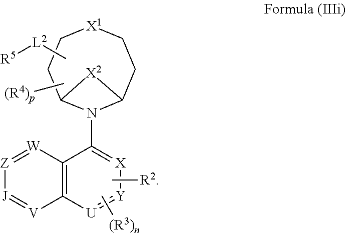

In some embodiments is a compound having the structure of Formula (IIIc), or a pharmaceutically acceptable salt or solvate thereof:

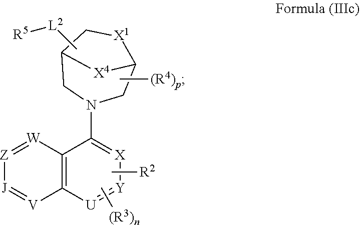

In some embodiments is a compound of Formula (IIIc), or a pharmaceutically acceptable salt or solvate thereof, wherein X⁴ is selected from —CH₂— and —CH₂CH₂—.

In some embodiments is a compound of Formula (IIIa), (IIIb), (IId), (IIIe), (IIIf), (IIIg), (IIIh), or (IIIi), or a pharmaceutically acceptable salt or solvate thereof, wherein X² is selected from —CH₂— and —CH₂CH₂—.

In another aspect, the disclosure provides a compound of Formula (IIIa)-(IIIi), or a pharmaceutically acceptable salt or solvate thereof:

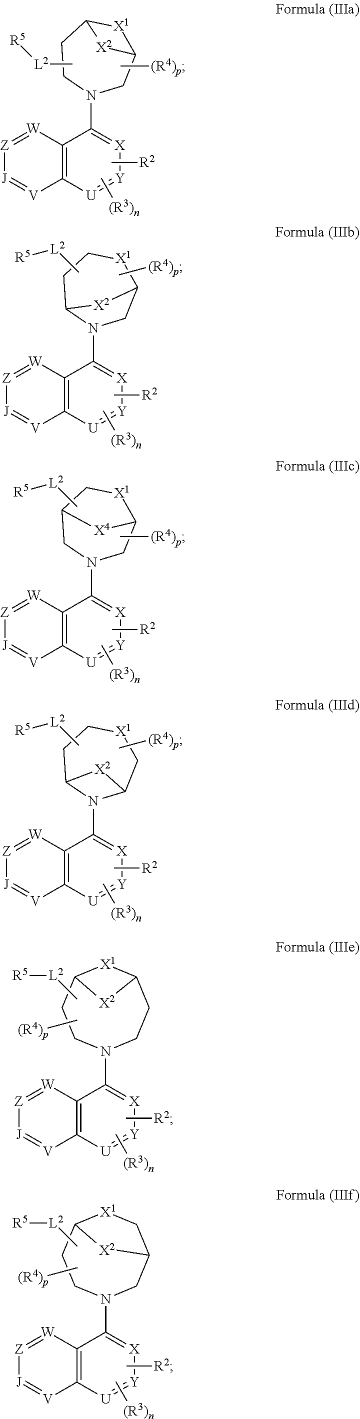

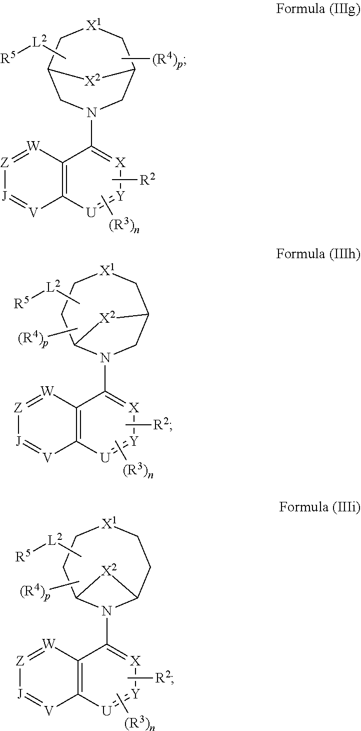

wherein:
X is C or N;
X¹ is selected from C(R⁴)(R⁶), N(R⁴), N(R⁶), O, S, S(O), and S(O)₂;
X² is selected from X³, —CH₂—, —X³CH₂, —CH₂X³—, —CH₂CH₂—, —C(H)(R⁴)—, —C(H)(R⁴)—, —X⁵C(H)(R⁴)—, —C(H)(R⁴)X⁵—, —C(H)(R⁴)CH₂—, —C(H)(R⁴)C(H)(R⁴)—, —C(R⁴)₂—, —X³C(R⁴)₂—, —C(R⁴)₂—X³—, —C(H)C(R⁴)₂, —C(R⁴)₂C(H)—, and —C(R⁴)₂—C(R⁴)₂—;
X³ is selected from N(R¹), O, S, S(O), and S(O)₂;
X⁴ is selected from X³, —CH₂, —X³CH₂—, —CH₂X³—, —CH₂CH₂, —C(H)(R⁴)—, —C(H)(R⁴)—, —X′C(H)(R⁴)—, —C(H)(R⁴)X³—, —C(H)(R⁴)CH₂—, —C(H)(R⁴)C(H)(R⁴)—, —C(R⁴)₂—, —X⁵C(R⁴)₂—, —C(R⁴)₂—X⁵—, —C(H)C(R⁴)₂—, —C(R⁴)₂C(H)—, and —C(R⁴)₂—C(R⁴)₂—;
X⁵ is selected from N(R¹), S, S(O), and S(O)₂;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a},
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound having the structure of Formula (IIIa), or a pharmaceutically acceptable salt or solvate thereof:

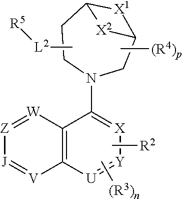

Formula (IIIa) wherein J, U, V, W, X, Y, Z, X¹, X², L², R², R³, n, R⁴, p, and R⁵ are as described herein.

In some embodiments is a compound having the structure of Formula (IIIb), or a pharmaceutically acceptable salt or solvate thereof:

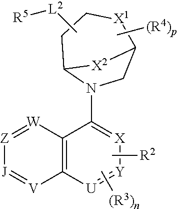

Formula (IIIb) wherein J, U, V, W, X, Y, Z, X¹, X², L², R², R³, n, R⁴, p, and R⁵ are as described herein.

In some embodiments is a compound having the structure of Formula (IIId), or a pharmaceutically acceptable salt or solvate thereof:

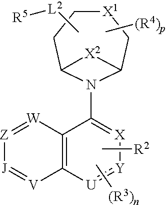

Formula (IIId) wherein J, U, V, W, X, Y, Z, X¹, X², L², R², R³, n, R⁴, p, and R⁵ are as described herein.

In some embodiments is a compound having the structure of Formula (IIIe), or a pharmaceutically acceptable salt or solvate thereof:

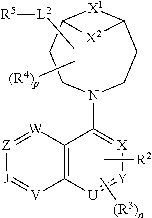

Formula (Ille) wherein J, U, V, W, X, Y, Z, X¹, X², L², R², R³, n, R⁴, p, and R⁵ are as described herein.

In some embodiments is a compound having the structure of Formula (IIIf), or a pharmaceutically acceptable salt or solvate thereof:

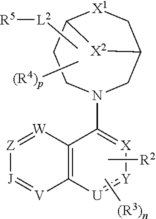

Formula (IIIf) wherein J, U, V, W, X, Y, Z, X¹, X², L², R², R³, n, R⁴, p, and R⁵ are as described herein.

In some embodiments is a compound having the structure of Formula (IIIg), or a pharmaceutically acceptable salt or solvate thereof:

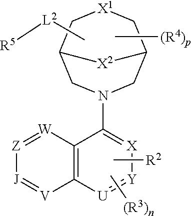

Formula (IIIg) wherein J, U, V, W, X, Y, Z, X¹, X², L², R², R³, n, R⁴, p, and R⁵ are as described herein.

In some embodiments is a compound having the structure of Formula (IIIh), or a pharmaceutically acceptable salt or solvate thereof:

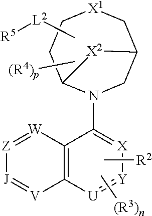

Formula (IIIh) wherein J, U, V, W, X, Y, Z, X¹, X², L², R², R³, n, R⁴, p, and R⁵ are as described herein.

In some embodiments is a compound having the structure of Formula (IIIi), or a pharmaceutically acceptable salt or solvate thereof:

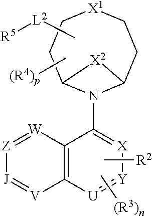

Formula (IIIi) wherein J, U, V, W, X, Y, Z, X¹, X², L², R², R³, n, R⁴, p, and R⁵ are as described herein.

In some embodiments is a compound having the structure of Formula (IIIc), or a pharmaceutically acceptable salt or solvate

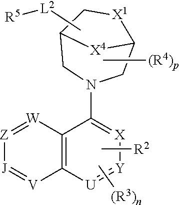

Formula (IIIc) wherein J, U, V, W, X, Y, Z, X¹, X⁴, L², R², R³, n, R⁴, p, and R⁵ are as described herein.

In another aspect, the disclosure provides a compound of Formula (IVa), (IVb), or (IVc), or a pharmaceutically acceptable salt or solvate thereof:

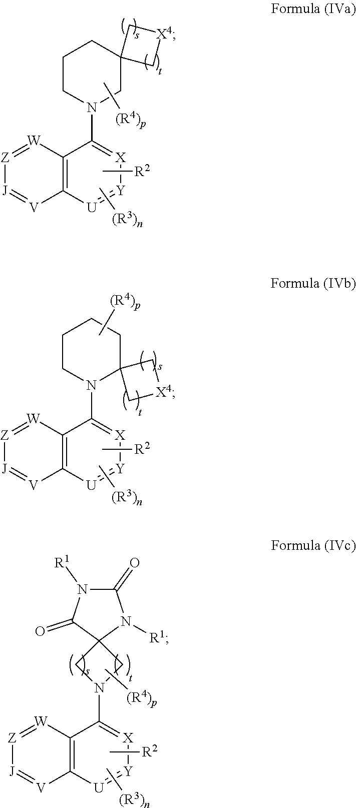

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O), C(O), or N;
U is C, S(O), S(O)₂, C(O), or N;
Z is Nor C(R⁸);
V and J are independently selected from N, C(R¹⁶), and C(R¹⁷), wherein one of V and J is C(R¹⁷);
W is N or C(R¹⁵);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)N(R¹⁴)—, —S(O)N(R¹⁴)—, —N(R¹⁴)S(O)₂—, —N(R¹⁴)S(O)—, —OCON(R¹⁴)—, —N(R¹⁴)C(O)O—, and —N(R¹)C(O)N(R¹)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

In some embodiments is a compound having the structure of Formula (IVa), or a pharmaceutically acceptable salt or solvate thereof:

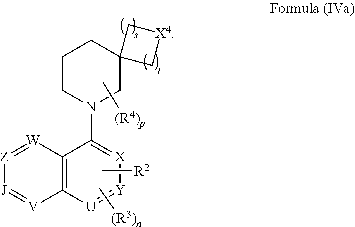

In some embodiments is a compound having the structure of Formula (IVb), or a pharmaceutically acceptable salt or solvate thereof:

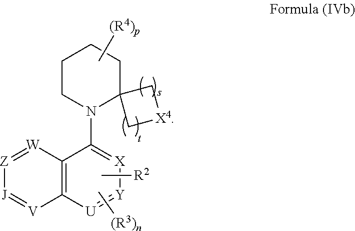

In some embodiments is a compound of Formula (IVa) or (IVb), or a pharmaceutically acceptable salt or solvate thereof, wherein s is 1.

In some embodiments is a compound of Formula (IVa) or (IVb), or a pharmaceutically acceptable salt or solvate thereof, wherein t is 1 or 2.

In some embodiments is a compound of Formula (IVa) or (IVb), or a pharmaceutically acceptable salt or solvate thereof, wherein X⁴ is N(R¹).

In some embodiments is a compound having the structure of Formula (IVc), or a pharmaceutically acceptable salt or solvate thereof:

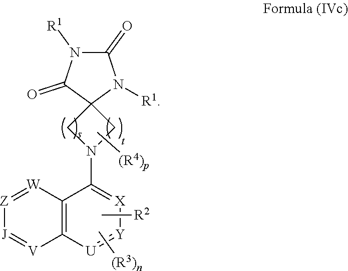

In another aspect, the disclosure provides a compound of Formula (Va), (Vb), or (Vc), or a pharmaceutically acceptable salt or solvate thereof:

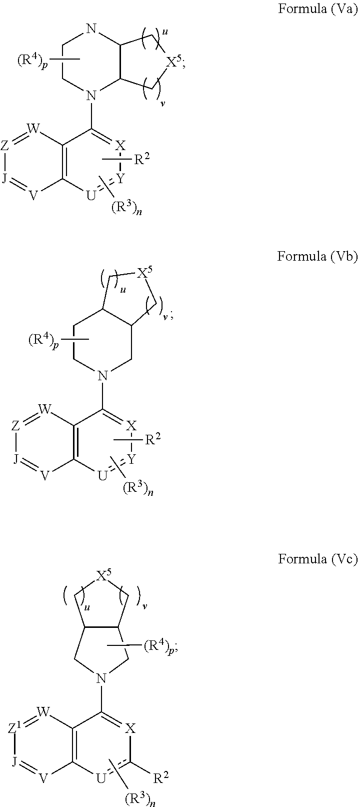

wherein:
X is C or N;
X³ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁴);
V and J are independently selected from N, C(R¹⁶), and C(R¹⁷), wherein one of V and J is C(R¹⁷);
W is N or C(R¹⁵);
L¹ and L² are independently selected from a bond, C₁-C₆alkyl, —O—, —N(R¹)—, —C(O)—, —N(R⁴)C(O)—, —C(O)N(R, —S—, —S(O)—, —S(O)—, —S(O)N(R¹)—, —S(O)N(R¹⁴)—, —N(R'S(O)—, —N(R¹⁴)S(O)—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

In some embodiments is a compound having the structure of Formula (Va), or a pharmaceutically acceptable salt or solvate thereof:

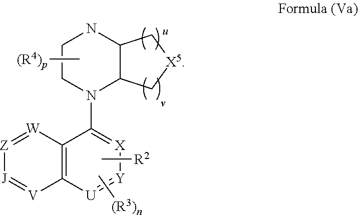

In some embodiments is a compound having the structure of Formula (Vb), or a pharmaceutically acceptable salt or solvate thereof:

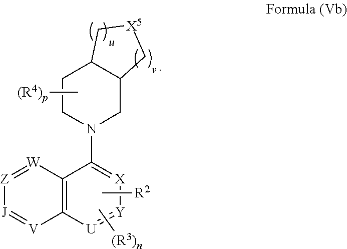

In some embodiments is a compound having the structure of Formula (Vc), or a pharmaceutically acceptable salt or solvate thereof:

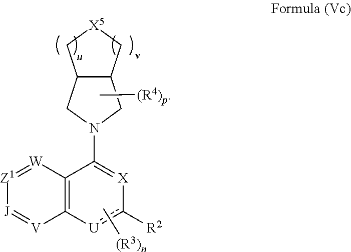

In some embodiments is a compound of Formula (Va), (Vb), or (Vc), or a pharmaceutically acceptable salt or solvate thereof, wherein u is 0 or 1.

In some embodiments is a compound of Formula (Va), (Vb), or (Vc), or a pharmaceutically acceptable salt or solvate thereof, wherein v is 0 or 1.

In some embodiments is a compound of Formula (Va), (Vb), or (Vc), or a pharmaceutically acceptable salt or solvate thereof, wherein X⁵ is N(R¹).

In some embodiments is a compound of Formula (IVa), (IVb), (IVc), (Va), (Vb), or (Vc), or a pharmaceutically acceptable salt or solvate thereof, wherein R¹ is hydrogen. In some embodiments is a compound of Formula (IVa), (IVb), (IVc), (Va), (Vb), or (Vc), or a pharmaceutically acceptable salt or solvate thereof, wherein R¹ is -L²-R⁵. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹ is hydrogen. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹ is -L²-R⁵. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein X⁴ is —NH—.

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein Y is C. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein Y is N. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein Y is C(O).

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein X is C. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein X is N.

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein U is C. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein U is N. In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein U is C(O).

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein Z is C(R⁸).

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R⁸ is hydrogen.

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein Z is N.

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein V is C(R¹⁶). In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein is C(H). In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein V is N.

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein J is C(R¹⁷).

In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein W is C(R¹⁸). In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein W is C(H). In some embodiments is a compound of Formula (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein W is N.

In some embodiments is a compound of Formula (I), (I′), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (I), (I′), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (I), (I′), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R² is —OR¹².

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹² is C₁₋₆alkyl optionally substituted with one, two, or three R^{20d}. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹² is C₂₋₉heterocycloalkyl optionally substituted with one, two, or three R^{20d}. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₄alkyl, —OR²¹, and —N(R²)(R²³).

In some embodiments is a compound of Formula (I), (I′), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R² is selected from

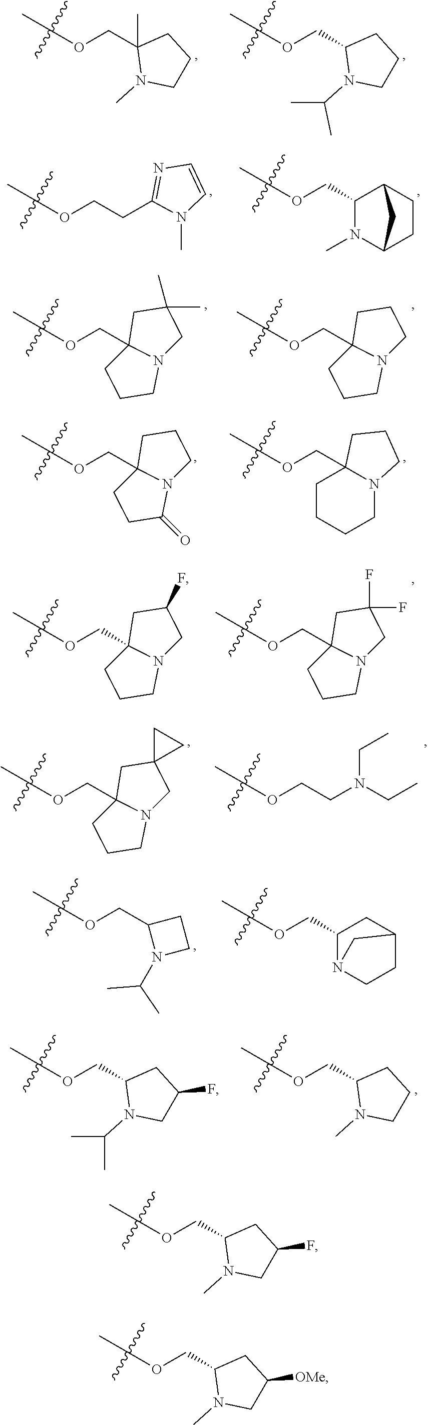

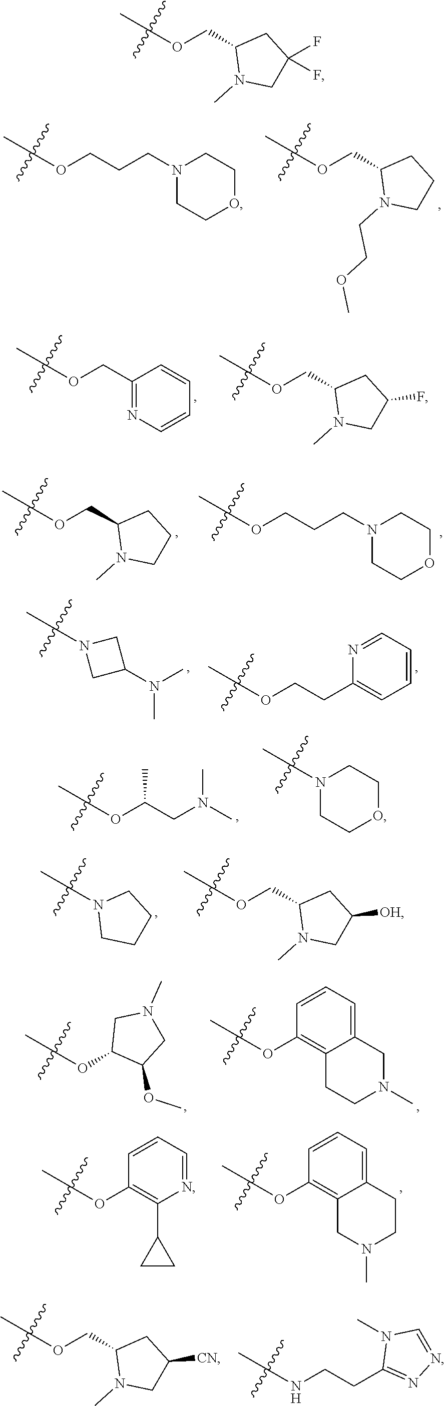

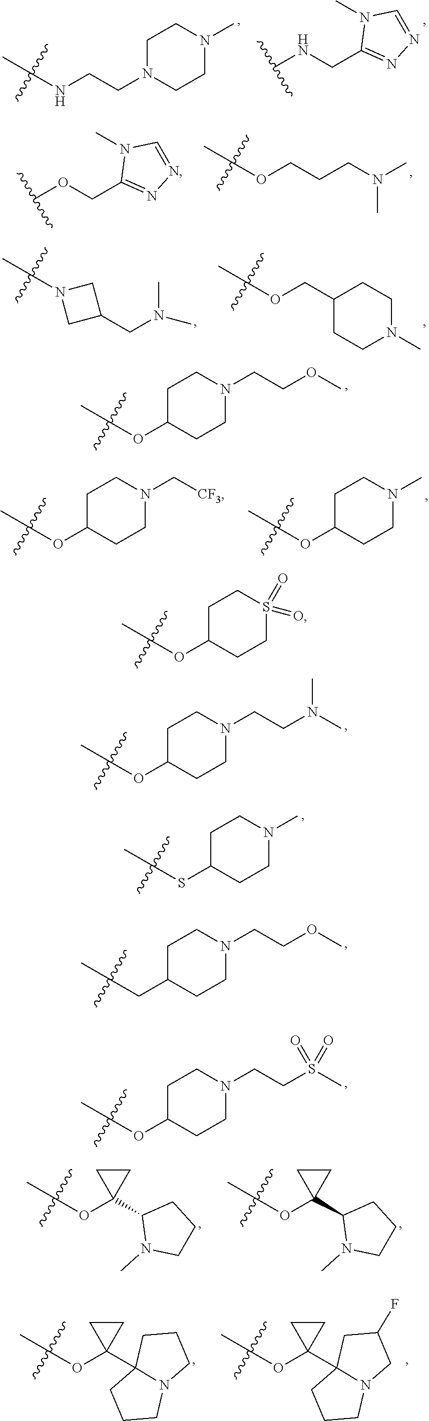

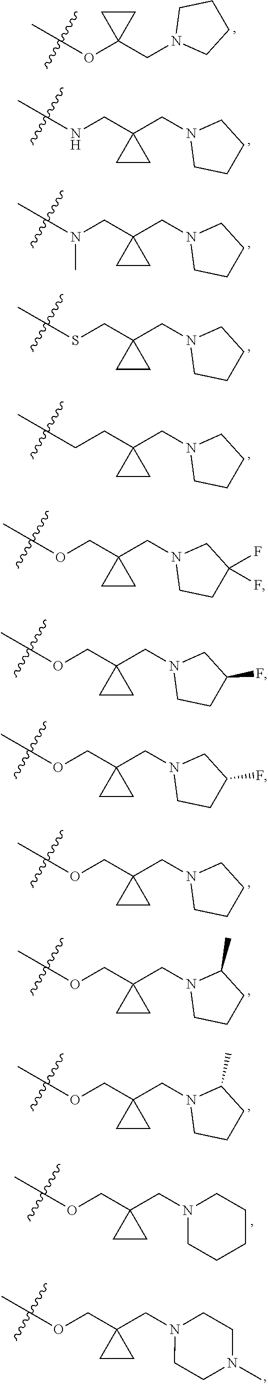

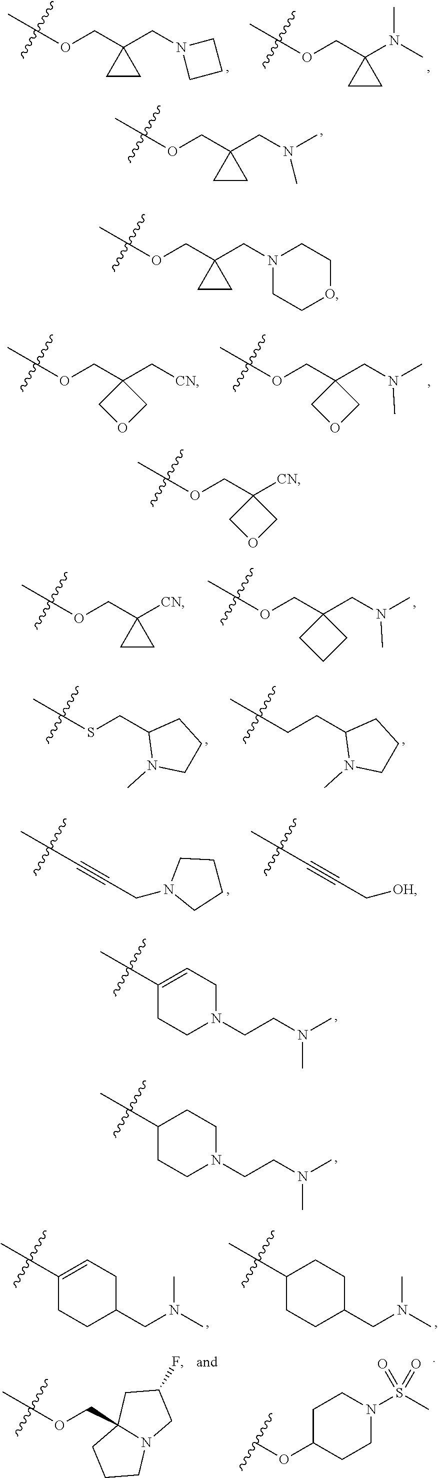

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein L¹ is a bond. In some embodiments is a compound of Formula (I), (I′), (I′), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein L¹ is O.

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹⁹ is C₁₋₉heteroaryl optionally substituted with one, two, or three R²⁰ⁱ. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R¹⁹ is C₆₋₁₀aryl optionally substituted with one, two, or three R²⁰ⁱ.

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein L² is selected from a bond, C₁-C₆alkyl, and —C(O)—. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein L² is a bond. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein L² is, C₁-C₆alkyl.

In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R⁵ is hydrogen. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R⁵ is a group other than an electrophilic moiety capable of forming a covalent bond with the cysteine residue at position 12 of a KRAS protein. In some embodiments is a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein R⁵ is —H, —NH₂, —OH, —NH(C₁₋₆ alkyl),

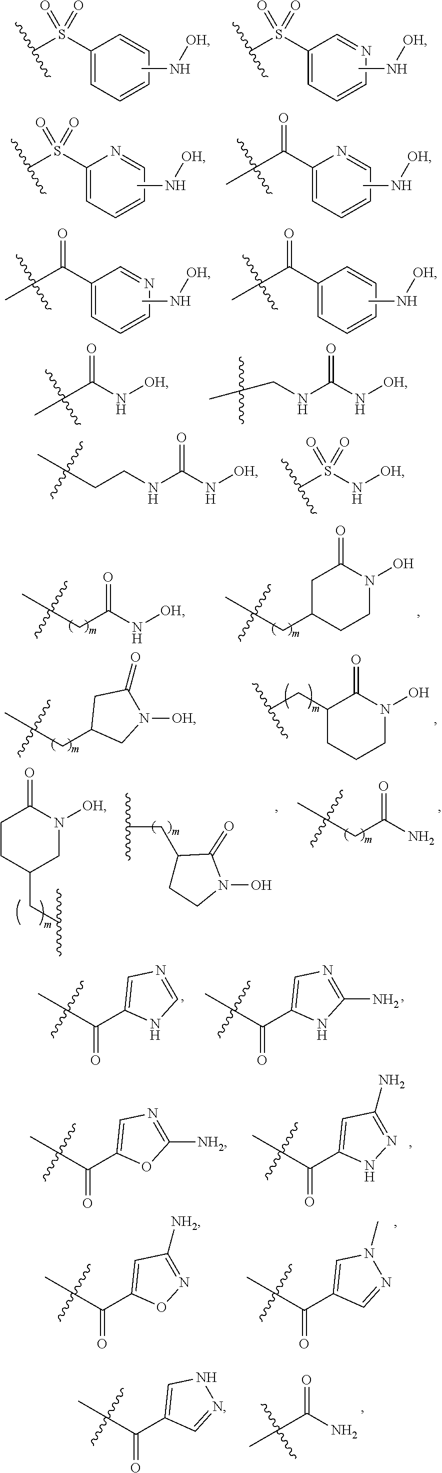

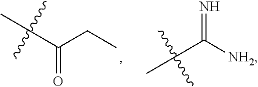

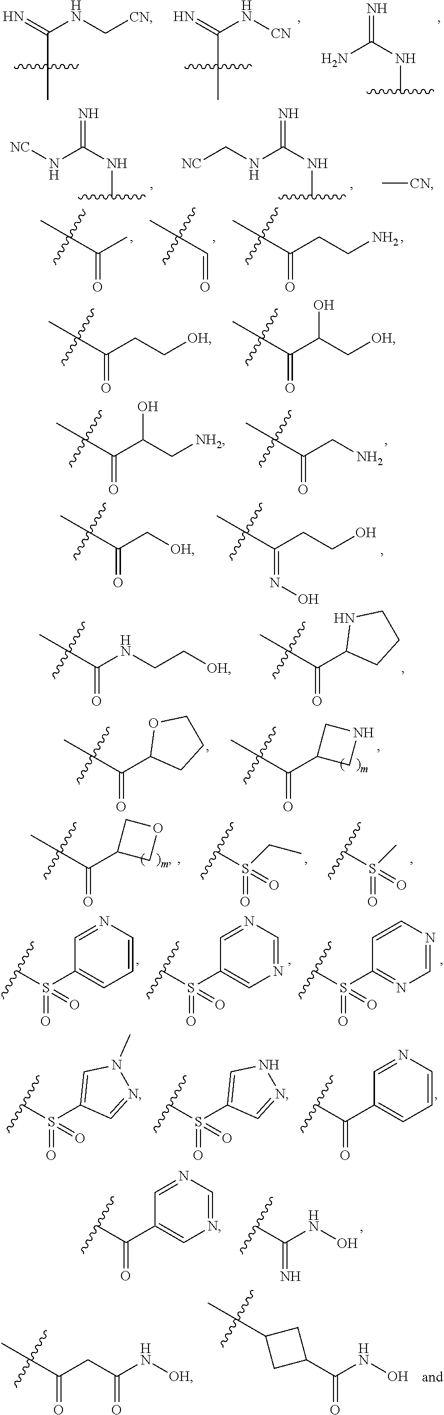

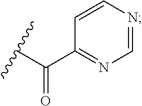 [SMILES: *C(=O)c1ccncn1]

and m, when present, is 0, 1, 2, or 3.

In another aspect, the disclosure provides a pharmaceutical composition comprising a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, and a pharmaceutically acceptable excipient.

In another aspect, the disclosure provides a method of treating cancer in a subject in need thereof, comprising administering to the subject a therapeutically effective amount of a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof. In some embodiments, the cancer is a solid tumor. In some embodiments, the cancer is a hematological cancer.

In another aspect, the disclosure provides a method of modulating activity of a Ras protein, comprising contacting a Ras protein with an effective amount of a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, thereby modulating the activity of the Ras protein. In some embodiments, said modulating comprises inhibiting the Ras protein activity. In some embodiments, the Ras protein is a K-Ras protein. In some embodiments, the Ras protein is a G12D or G12V mutant K-Ras. In some embodiments, said method comprises administering an additional agent or therapy. In some embodiments, the additional agent or therapy is selected from the group consisting of a chemotherapeutic agent, a radioactive agent, and an immune modulator. In some embodiments, said modulating takes place in vitro or in vivo.

In another aspect, the disclosure provides a method of inhibiting cell growth, comprising administering a cell expressing a Ras protein with an effective amount of a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, thereby inhibiting growth of said cells. In some embodiments, the method comprises administering an additional agent to said cell. In some embodiments, the additional agent is a chemotherapeutic agent, a radioactive agent, or an immune modulator.

In another aspect, the disclosure provides a Ras protein bound by a compound of Formula (I), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein activity of said Ras protein is reduced as compared to a Ras protein unbound to said compound.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).
Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to a straight or branched hydrocarbon chain radical consisting solely of carbon and hydrogen atoms, containing no unsaturation. In some embodiments, the “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a straight or branched hydrocarbon chain radical group consisting solely of carbon and hydrogen atoms, containing at least one carbon-carbon triple bond. Non-limiting examples of an alkynyl group include —C≡CH, —C≡CCH₃, —C≡CCH₂CH₃ and —C≡CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized x-electron system containing 4n+2π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group). In embodiments, the aryl radical is a monocyclic, bicyclic, or tricyclic ring system. In some embodiments is a “fused ring aryl” wherein the aryl ring is fused with a cycloalkyl or a heterocycloalkyl ring.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

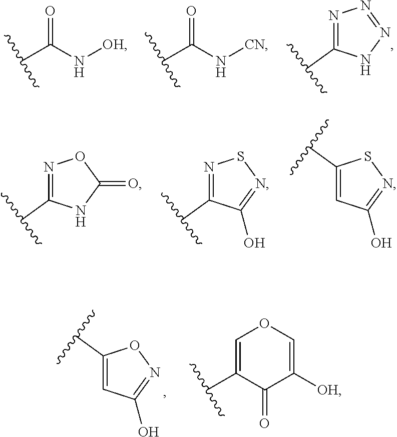

and the like.

The term “cycloalkyl” refers to a monocyclic or polycyclic non-aromatic radical, wherein each of the atoms forming the ring (i.e. skeletal atoms) is a carbon atom. Cycloalkyls may be saturated or partially unsaturated. In some embodiments, a cycloalkyl ring is fused with an aryl, heteroaryl, heterocycloalkyl, or a second cycloalkyl ring. In some embodiments, a cycloalkyl ring is a spirocyclic cycloalkyl ring. In some embodiments, cycloalkyl groups include groups having from 3 to 10 ring atoms. Depending on the structure, a cycloalkyl group can be a monoradical or a diradical (i.e., a cycloalkylene group).

The terms “heteroaryl” or, alternatively, “heteroaromatic” refers to an aryl group that includes one or more ring heteroatoms selected from nitrogen, oxygen and sulfur. As used herein, the heteroaryl radical is a monocyclic, bicyclic, or tricyclic ring system, wherein at least one of the rings in the ring system is fully unsaturated. An N-containing “heteroaromatic” or “heteroaryl” moiety refers to an aromatic group in which at least one of the skeletal atoms of the ring is a nitrogen atom. Depending on the structure, a heteroaryl group can be a monoradical or a diradical (i.e., a heteroarylene group). In some embodiments is a “fused ring heteroaryl” wherein the heteroaryl ring is fused with a cycloalkyl or heterocycloalkyl ring.

A “heterocycloalkyl” group or “heteroalicyclic” group refers to a cycloalkyl group, wherein at least one skeletal ring atom is a heteroatom selected from nitrogen, oxygen and sulfur. Heterocycloalkyls may be saturated or partially unsaturated. In some embodiments, a heterocycloalkyl ring is fused with an aryl, heteroaryl, cycloalkyl, or a second heterocycloalkyl ring. The term heterocycloalkyl also includes all ring forms of the carbohydrates, including but not limited to the monosaccharides, the disaccharides and the oligosaccharides. In some embodiments, a heterocycloalkyl ring is a spirocyclic heterocycloalkyl ring. In some embodiments, a heterocycloalkyl ring is a bridged heterocycloalkyl ring. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Depending on the structure, a heterocycloalkyl group can be a monoradical or a diradical (i.e., a heterocycloalkylene group).

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “oxo” refers to the ═O radical.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

The term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R⁴)₂), and the protected derivatives thereof. By way of example, an optional substituents may be LSRs, wherein each Ls is independently selected from a bond, —O—, —C(═O)—, —S—, —S(═O)—, —S(═O)₂—, —NH—, —NHC(O)—, —C(O)NH—, S(═O)₂NH—, —NHS(—O)₂, —OC(O)NH—, —NHC(O)O—, —(C₁-C₆alkyl)-, or —(C₂-C₆alkenyl)-; and each R$ is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

An “antigen” is a moiety or molecule that contains an epitope, and, as such, also specifically binds to an antibody.

An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, i.e., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH—VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

The terms “Mutant Ras” and “Ras mutant,” as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids). In some embodiments, two or more mutations can be consecutive, non-consecutive, or a combination thereof. In some embodiments, a mutation can be present at any position of Ras. In some embodiments, a mutation can be present at position 12, 13, 62, 92, 95, or any combination thereof of Ras relative to SEQ ID No. 1 when optimally aligned. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169, 168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

### Compounds

The compounds of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XIIb-2), (XXIIc), (XXIId), (XXIle), (XXIIf), (XXIIg), (XXIII-1), (XXIII-2), (XXIV-1), or (XXIV-2), or a pharmaceutically acceptable salt or solvate thereof, are Ras modulators (including Ras inhibitors) and have a wide range of applications in therapeutics, diagnostics, and other biomedical research.

In an aspect is provided a compound of Formula (I-1), or a pharmaceutically acceptable salt or solvate thereof:

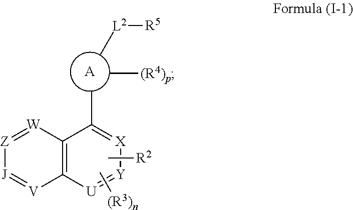

wherein:

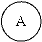

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(—NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In one aspect, the disclosure provides a compound of Formula (I-2), or a pharmaceutically acceptable salt or solvate thereof:

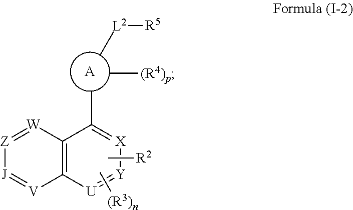

wherein:

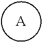

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound of Formula (I-1) or (I-2) having the structure of Formula (I′), or a pharmaceutically acceptable salt or solvate thereof:

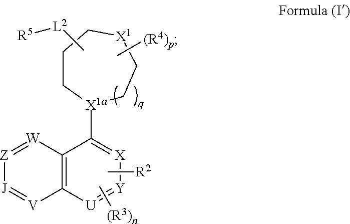

wherein X¹ is selected from C, N, O, S, S(O), and S(O)₂; X^{1a} is selected from N and C(H); and q is 1 or 2. In some embodiments is a compound of Formula (I-1) or (I-2) having the structure of Formula (I′), or a pharmaceutically acceptable salt or solvate thereof, wherein X¹ is selected from C, N, O, S, S(O), and S(O)₂; X^{1a} is selected from N and C(H); q is 1 or 2; and p is 2, 2-5, 2-4, or 2-3.

In some embodiments is a compound of Formula (I-1) or (I-2) having the structure of Formula (Ia), or a pharmaceutically acceptable salt or solvate thereof:

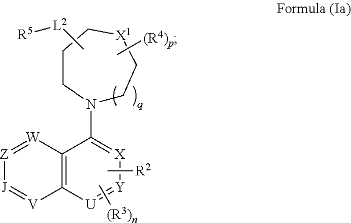

wherein X¹ is selected from C, N, O, S, S(O), and S(O)₂; and q is 1 or 2. In some embodiments is a compound of Formula (Ia), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I-1) or (I-2) having the structure of Formula (Ib), or a pharmaceutically acceptable salt or solvate thereof:

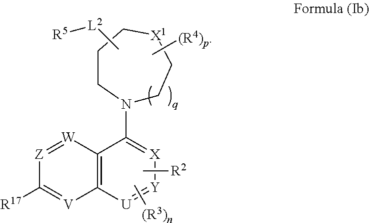

In some embodiments is a compound of Formula (I-1) or (I-2) having the structure of Formula (Ic), or a pharmaceutically acceptable salt or solvate thereof:

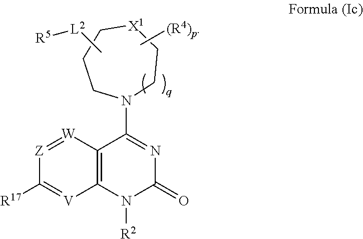

In some embodiments is a compound of Formula (Ic), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I-1) or (I-2) having the structure of Formula (Id), or a pharmaceutically acceptable salt or solvate thereof:

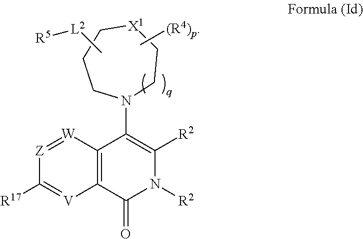

In some embodiments is a compound of Formula (Id), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I-1) or (I-2) having the structure of Formula (Ie), or a pharmaceutically acceptable salt or solvate thereof:

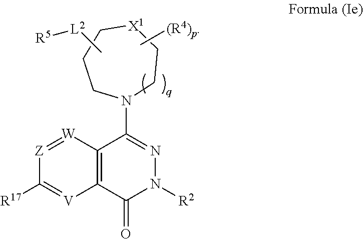

In some embodiments is a compound of Formula (Ie), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I-1) or (I-2) having the structure of Formula (If), or a pharmaceutically acceptable salt or solvate thereof:

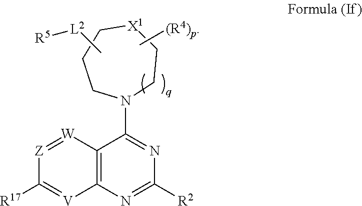

In some embodiments is a compound of Formula (If), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I-1) or (I-2) having the structure of Formula (Ig), or a pharmaceutically acceptable salt or solvate thereof:

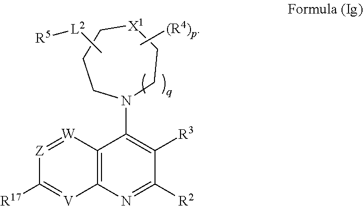

In some embodiments is a compound of Formula (Ig), or a pharmaceutically acceptable salt or solvate thereof, wherein X¹ is selected from C, N, O, S, S(O), and S(O)₂; q is 1 or 2; and p is 2, 2-5, 2-4, or 2-3.

In some embodiments is a compound of Formula (I′), (Ia), (Ib), (Ic), (Id), (Ie), (If), or (Ig), or a pharmaceutically acceptable salt or solvate thereof, wherein X¹ is N(H). In some embodiments is a compound of Formula (I′), (Ia), (Ib), (Ic), (Id), (Ie), (If), or (Ig), or a pharmaceutically acceptable salt or solvate thereof, wherein X¹ is N(R⁴). In some embodiments is a compound of Formula (I′), (Ia), (Ib), (Ic), (Id), (Ie), (If), or (Ig), or a pharmaceutically acceptable salt or solvate thereof, wherein X¹ is N(-L²-R⁵), and wherein R⁵ is hydrogen, or a group other than an electrophilic moiety capable of forming a covalent bond with the cysteine residue at position 12 of a KRAS protein. In some embodiments is a compound of Formula (I′), (Ia), (Ib), (Ic), (Id), (Ie), (If), or (Ig), or a pharmaceutically acceptable salt or solvate thereof, wherein X¹ is N(-L²-R⁵), and wherein R⁵ is a group having an electrophilic moiety capable of forming a covalent bond with a residue at position 12 of a KRAS protein, e.g., the cysteine residue at position 12. In some embodiments is a compound of Formula (I′), (Ia), (Ib), (Ic), (Id), (Ie), (If), or (Ig), or a pharmaceutically acceptable salt or solvate thereof, wherein X¹ is O. In some embodiments is a compound of Formula (I′), (Ia), (Ib), (Ic), (Id), (Ie), (If), or (Ig), or a pharmaceutically acceptable salt or solvate thereof, wherein X¹ is S. In some embodiments is a compound of Formula (I′), (Ia), (Ib), (Ic), (Id), (Ie), (If), or (Ig), or a pharmaceutically acceptable salt or solvate thereof, wherein X¹ is S(O)₂. In some embodiments is a compound of Formula (I′), (Ia), (Ib), (Ic), (Id), (Ie), (If), or (Ig), or a pharmaceutically acceptable salt or solvate thereof, wherein X¹ is CH₂. In some embodiments is a compound of Formula (I′), (Ia), (Ib), (Ic), (Id), (Ie), (If), or (Ig), or a pharmaceutically acceptable salt or solvate thereof, wherein X¹ is C(H)(R⁴). In some embodiments is a compound of Formula (I′), (Ia), (Ib), (Ic), (Id), (Ie), (If), or (Ig), or a pharmaceutically acceptable salt or solvate thereof, wherein X¹ is C(R⁴)₂. In some embodiments is a compound of Formula (I′), (Ia), (Ib), (Ic), (Id), (Ie), (If), or (Ig), or a pharmaceutically acceptable salt or solvate thereof, wherein X¹ is C(H)(-L²-R⁵).

In some embodiments is a compound of Formula (I′), (Ia), (Ib), (Ic), (Id), (Ie), (If), or (Ig), or a pharmaceutically acceptable salt or solvate thereof, wherein q is 1. In some embodiments is a compound of Formula (I′), (Ia), (Ib), (Ic), (Id), (Ie), (If), or (Ig), or a pharmaceutically acceptable salt or solvate thereof, wherein q is 2.

In some embodiments is a compound of Formula (I-1), (I-2), (I′), (Ia), (Ib), (Ic), (Id), (Ie), (If), or (Ig), or a pharmaceutically acceptable salt or solvate thereof, wherein V is C(R¹⁶). In some embodiments is a compound of Formula (I-1), (I-2), (I′), (Ia), (Ib), (Ic), (Id), (Ie), (If), or (Ig), or a pharmaceutically acceptable salt or solvate thereof, wherein V is C(H). In some embodiments is a compound of Formula (I-1), (I-2), (I′), (Ia), (Ib), (Ic), (Id), (Ie), (If), or (Ig), or a pharmaceutically acceptable salt or solvate thereof, wherein V is N.

In some embodiments is a compound of Formula (I-1), (I-2), (I′), (Ia), (Ib), (Ic), (Id), (Ie), (If), or (Ig), or a pharmaceutically acceptable salt or solvate thereof, wherein W is C(R¹⁸). In some embodiments is a compound of Formula (I-1), (I-2), (I′), (Ia), (Ib), (Ic), (Id), (Ie), (If), or (Ig), or a pharmaceutically acceptable salt or solvate thereof, wherein W is C(H). In some embodiments is a compound of Formula (I-1), (I-2), (I′), (Ia), (Ib), (Ic), (Id), (Ie), (If), or (Ig), or a pharmaceutically acceptable salt or solvate thereof, wherein W is N.

In some embodiments is a compound of Formula (I-1), (1-2), (I′), (Ia), (Ib), (Ic), (Id), (Ie), (If), or (Ig), or a pharmaceutically acceptable salt or solvate thereof, wherein Z is C(R⁸). In some embodiments is a compound of Formula (I-1), (I-2), (I′), (Ia), (Ib), (Ic), (Id), (Ie), (If), or (Ig), or a pharmaceutically acceptable salt or solvate thereof, wherein Z is C(H). In some embodiments is a compound of Formula (I-1), (I-2), (I′), (Ia), (Ib), (Ic), (Id), (Ie), (If), or (Ig), or a pharmaceutically acceptable salt or solvate thereof, wherein Z is N.

In some embodiments is a compound of Formula (I-1), (I-2), (I′), (Ia), (Ib), (Ic), (Id), (Ie), (If), or (Ig), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (I-1), (I-2), (I′), (Ia), (Ib), (Ic), (Id), (Ie), (If), or (Ig), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (I-1), (I-2), (I′), (Ia), (Ib), (Ic), (Id), (Ie), (If), or (Ig), or a pharmaceutically acceptable salt or solvate thereof, wherein R² is —OR¹².

In some embodiments is a compound of Formula (I-1), (1-2), (I′), (Ia), (Ib), (Ic), (Id), (Ie), (If), or (Ig), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (I-1), (I-2), (I′), (Ia), (Ib), (Ic), (Id), (Ie), (If), or (Ig), or a pharmaceutically acceptable salt or solvate thereof, wherein R¹² is C₁₋₆alkyl optionally substituted with one, two, or three R^{20d}. In some embodiments is a compound of Formula (I-1), (I-2), (I′), (Ia), (Ib), (Ic), (Id), (Ie), (If), or (Ig), or a pharmaceutically acceptable salt or solvate thereof, wherein R¹² is C₂₋₉heterocycloalkyl optionally substituted with one, two, or three R^{20d}. In some embodiments is a compound of Formula (I-1), (I-2), (I′), (Ia), (Ib), (Ic), (Id), (Ie), (If), or (Ig), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (I-1), (I-2), (I′), (Ia), (Ib), (Ic), (Id), (Ie), (If), or (Ig), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (I-1), (I-2), (I′), (Ia), (Ib), (Ic), (Id), (Ie), (If), or (Ig), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (I-1), (I-2), (I′), (Ia), (Ib), (Ic), (Id), (Ie), (If), or (Ig), or a pharmaceutically acceptable salt or solvate thereof, wherein R² is selected from

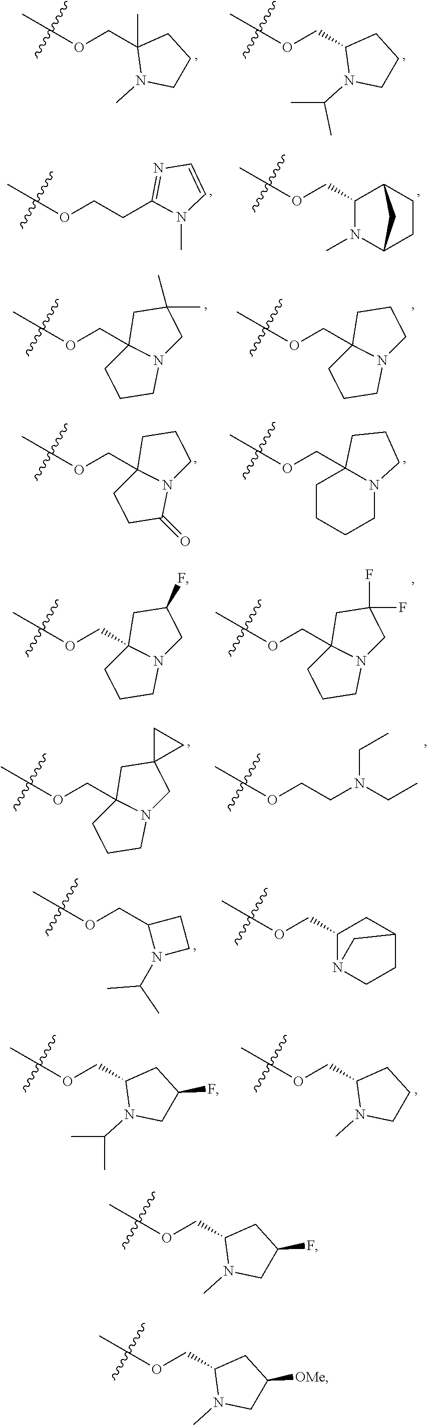

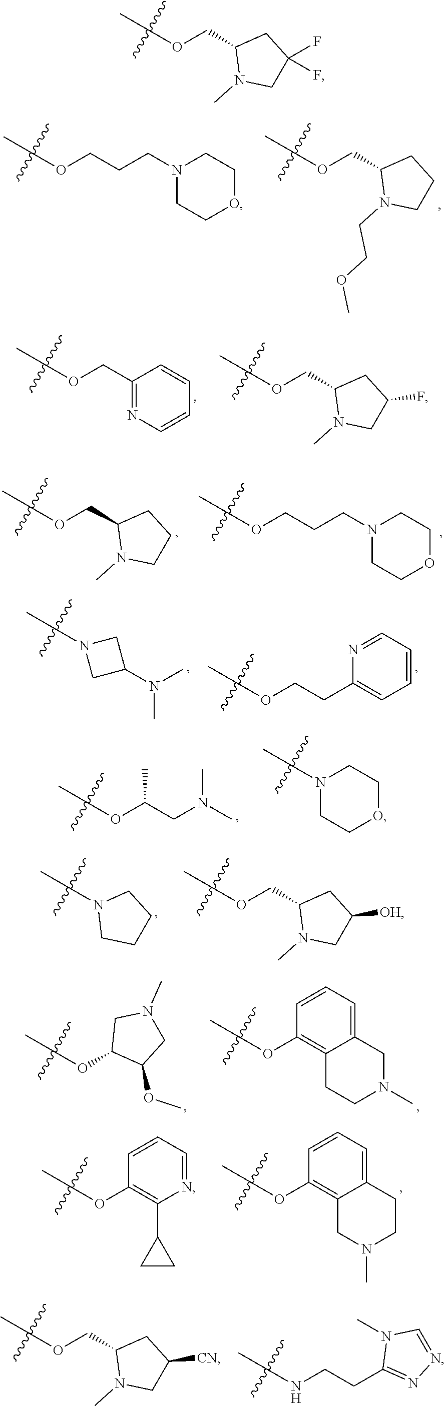

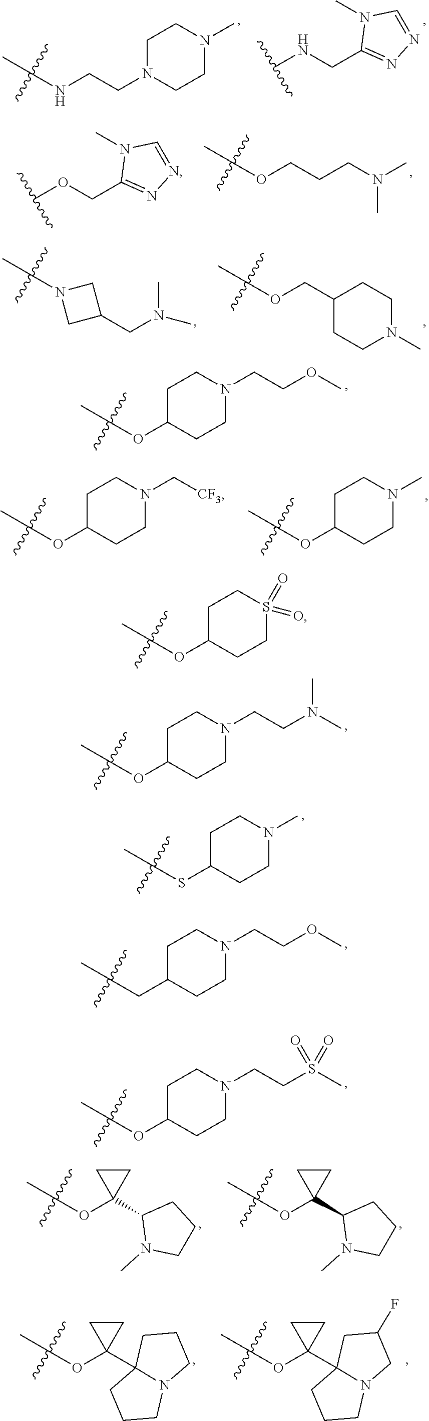

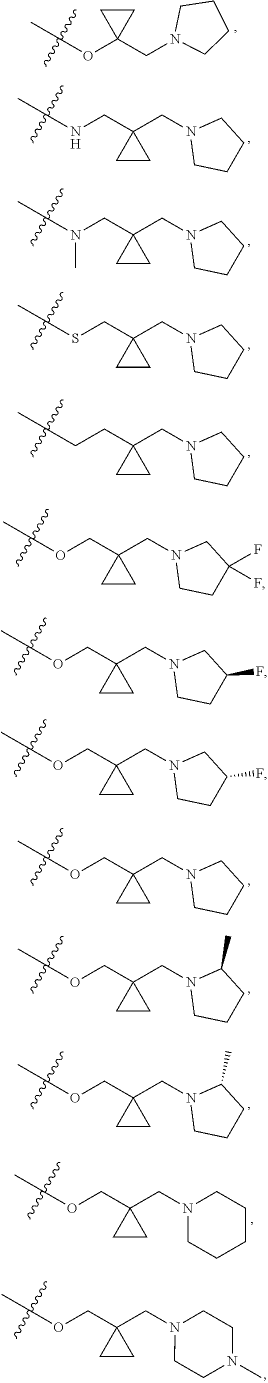

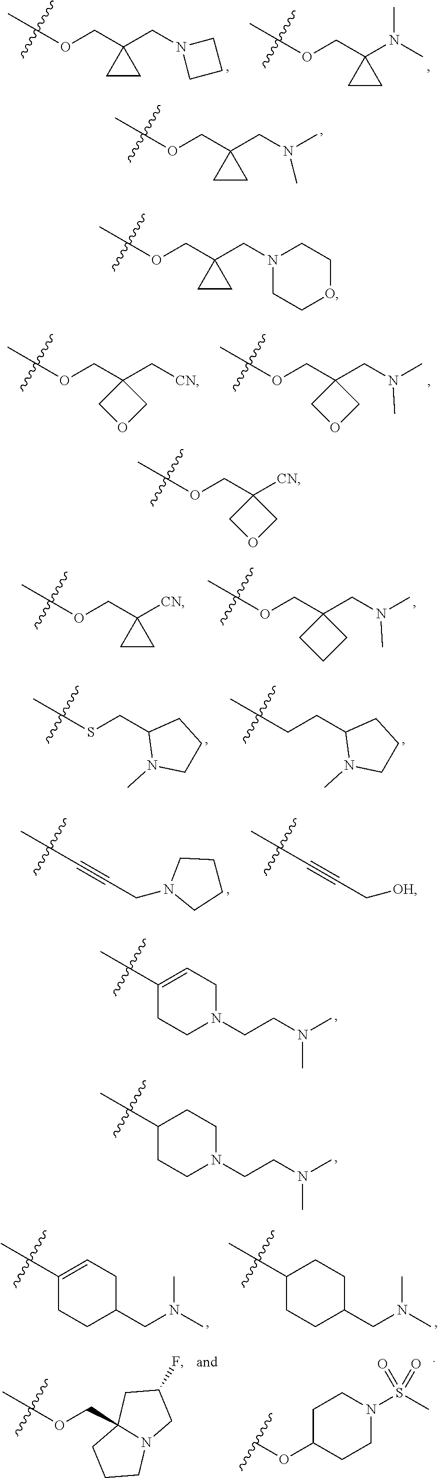

In some embodiments is a compound of Formula (I-1), (1-2), (I′), (Ia), (Ib), (Ic), (Id), (Ie), (If), or (Ig), or a pharmaceutically acceptable salt or solvate thereof, wherein L¹ is a bond. In some embodiments is a compound of Formula (I-1), (I-2), (I′), (Ia), (Ib), (Ic), (Id), (Ie), (If), or (Ig), or a pharmaceutically acceptable salt or solvate thereof, wherein L¹ is O.

In some embodiments is a compound of Formula (I-1), (I-2), (I′), (Ia), (Ib), (Ic), (Id), (Ie), (If), or (Ig), or a pharmaceutically acceptable salt or solvate thereof, wherein R¹⁹ is C₁₋₉heteroaryl optionally substituted with one, two, or three R²⁰ⁱ. In some embodiments is a compound of Formula (I-1), (I-2), (I′), (Ia), (Ib), (Ic), (Id), (Ie), (If), or (Ig), or a pharmaceutically acceptable salt or solvate thereof, wherein R¹⁹ is C₆₋₁₀aryl optionally substituted with one, two, or three R²⁰ⁱ.

In some embodiments is a compound of Formula (I-1), (I-2), (I′), (Ia), (Ib), (Ic), (Id), (Ie), (If), or (Ig), or a pharmaceutically acceptable salt or solvate thereof, wherein L² is selected from a bond, C₁-C₆alkyl, and —C(O)—. In some embodiments is a compound of Formula (I-1), (I-2), (I′), (Ia), (Ib), (Ic), (Id), (Ie), (If), or (Ig), or a pharmaceutically acceptable salt or solvate thereof, wherein L² is a bond. In some embodiments is a compound of Formula (I-1), (I-2), (I′), (Ia), (Ib), (Ic), (Id), (Ie), (If), or (Ig), or a pharmaceutically acceptable salt or solvate thereof, wherein L² is, C₁-C₆alkyl.

In some embodiments is a compound of Formula (I-1), (I′), (Ia), (Ib), (Ic), (Id), (Ie), (If), or (Ig), or a pharmaceutically acceptable salt or solvate thereof, wherein R⁵ is hydrogen. In some embodiments is a compound of Formula (I-1), (I′), (Ia), (Ib), (Ic), (Id), (Ie), (If), or (Ig), or a pharmaceutically acceptable salt or solvate thereof, wherein R⁵ is a group other than an electrophilic moiety capable of forming a covalent bond with the cysteine residue at position 12 of a KRAS protein. In some embodiments is a compound of Formula (I-1), (I′), (Ia), (Ib), (Ic), (Id), (Ie), (If), or (Ig), or a pharmaceutically acceptable salt or solvate thereof, wherein R⁵ is —H, —NH₂, —OH, —NH(C₁₋₆ alkyl).

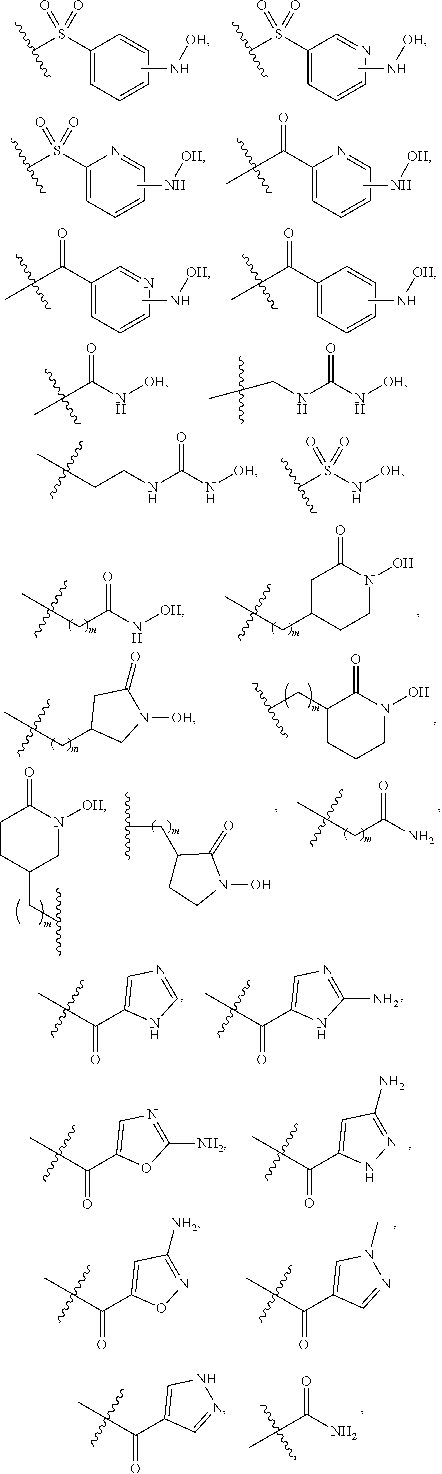

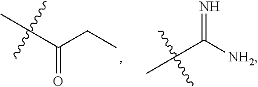

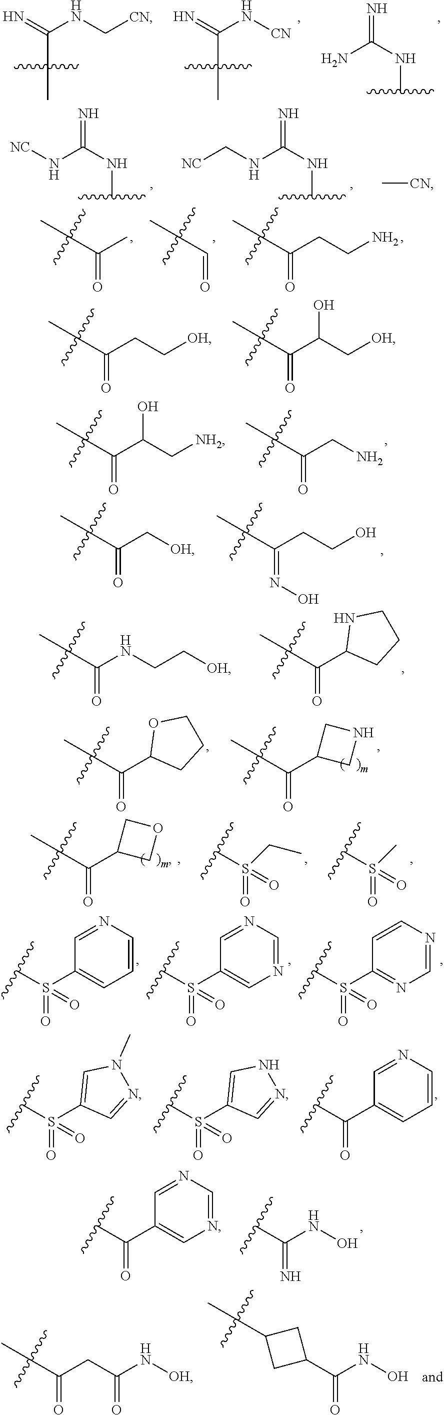

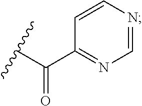 [SMILES: *C(=O)c1ccncn1]

and m, when present, is 0, 1, 2, or 3.

In some embodiments of the subject compound of Formula (I-1), (I′), (Ia), (Ib), (Ic), (Id), (Ie), (If), or (Ig), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C_{2- 9}heterocycloalkyl, C₆₋₁₀aryl, and C₁₋₉heteroaryl are optionally substituted with one, two, or three R^{20a}. In some embodiments of the subject compound of Formula (I-1), (I′), (Ia), (Ib), (Ic), (Id), (Ie), (If), or (Ig), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (I-1), (I′), (Ia), (Ib), (Ic), (Id), (Ie), (If), or (Ig), or a pharmaceutically acceptable salt or solvate thereof, R⁵ is halogen. In further embodiments of the subject compound of Formula (I-1), (I′), (Ia), (Ib), (Ic), (Id), (Ie), (If), or (Ig), or a pharmaceutically acceptable salt or solvate thereof, R⁵ is —CN. In embodiments of the subject compound of Formula (I-1), (I′), (Ia), (Ib), (Ic), (Id), (Ie), (If), or (Ig), or a pharmaceutically acceptable salt or solvate thereof, R⁵ is —OH. In some embodiments of the subject compound of Formula (I-1), (I′), (Ia), (Ib), (Ic), (Id), (Ie), (If), or (Ig), or a pharmaceutically acceptable salt or solvate thereof, R⁵ is —NH₂. In further embodiments of the subject compound of Formula (I-1), (I′), (Ia), (Ib), (Ic), (Id), (Ie), (If), or (Ig), or a pharmaceutically acceptable salt or solvate thereof, R⁵ is —C(O)OH. In select embodiments of the subject compound of Formula (I-1), (I′), (Ia), (Ib), (Ic), (Id), (Ie), (If), or (Ig), or a pharmaceutically acceptable salt or solvate thereof, R⁵ is —OC(O)NH₂. In embodiments of the subject compound of Formula (I-1), (I′), (Ia), (b), (Ic), (Id), (Ie), (If), or (Ig), or a pharmaceutically acceptable salt or solvate thereof, R⁵ is —NHC(O)NH₂. In embodiments of the subject compound of Formula (I-1), (I′), (Ia), (Ib), (Ic), (Id), (Ie), (If), or (Ig), or a pharmaceutically acceptable salt or solvate thereof, R⁵ is —NHC(O)OH. In some embodiments of the subject compound of Formula (I-1), (I′), (Ia), (Ib), (Ic), (Id), (Ie), (If), or (Ig), or a pharmaceutically acceptable salt or solvate thereof, R⁵ is —NHS(O)₂H. In embodiments of the subject compound of Formula (I-1), (I′), (Ia), (Ib), (Ic), (Id), (Ie), (If), or (Ig), or a pharmaceutically acceptable salt or solvate thereof, R⁵ is —C(O)NH₂. In some embodiments of the subject compound of Formula (I-1), (I′), (Ia), (Ib), (Ic), (Id), (Ie), (If), or (Ig), or a pharmaceutically acceptable salt or solvate thereof, R⁵ is hydrogen. In embodiments of the subject compound of Formula (I-1), (I′), (Ia), (Ib), (Ic), (Id), (Ie), (If), or (Ig), or a pharmaceutically acceptable salt or solvate thereof, R⁵ is oxo. In further embodiments of the subject compound of Formula (I-1), (I′), (Ia), (Ib), (Ic), (Id), (Ie), (If), or (Ig), or a pharmaceutically acceptable salt or solvate thereof, R⁵ is C₁₋₆alkyl optionally substituted with one, two, or three R^{20a}. In select embodiments of the subject compound of Formula (I-1), (I′), (Ia), (Ib), (Ic), (Id), (Ie), (If), or (Ig), or a pharmaceutically acceptable salt or solvate thereof, R⁵ is C₃₋₆cycloalkyl optionally substituted with one, two, or three R^{20a}. In embodiments of the subject compound of Formula (I-1), (I′), (Ia), (Ib), (Ic), (Id), (Ie), (If), or (Ig), or a pharmaceutically acceptable salt or solvate thereof, R⁵ is C₂₋₉heterocycloalkyl optionally substituted with one, two, or three R^{20a}. In some embodiments of the subject compound of Formula (I-1), (I′), (Ia), (Ib), (Ic), (Id), (Ie), (If), or (Ig), or a pharmaceutically acceptable salt or solvate thereof, R⁵ is C₆₋₁₀aryl optionally substituted with one, two, or three R^{20a}. In further embodiments of the subject compound of Formula (I-1), (I′), (Ia), (Ib), (Ic), (Id), (Ie), (If), or (Ig), or a pharmaceutically acceptable salt or solvate thereof, R⁵ is C₁₋₉heteroaryl optionally substituted with one, two, or three R^{20a}. In select embodiments of the subject compound of Formula (I-1), (I′), (Ia), (Ib), (Ic), (Id), (Ie), (If), or (Ig), or a pharmaceutically acceptable salt or solvate thereof, R⁵ is —OR¹². In embodiments of the subject compound of Formula (I-1), (I′), (Ia), (Ib), (Ic), (Id), (Ie), (If), or (Ig), or a pharmaceutically acceptable salt or solvate thereof, R⁵ is —N(R¹²)(R¹³). In select embodiments of the subject compound of Formula (I-1), (I′), (Ia), (Ib), (Ic), (Id), (Ie), (If), or (Ig), or a pharmaceutically acceptable salt or solvate thereof, R⁵ is N(R¹⁴)S(O)₂R¹⁵. In select embodiments of the subject compound of Formula (I-1), (I′), (Ia), (Ib), (Ic), (Id), (Ie), (If), or (Ig), or a pharmaceutically acceptable salt or solvate thereof, R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}. In some embodiments is a compound of Formula (I-2), (I′), (Ia), (Ib), (Ic), (Id), (Ie), (If), or (Ig), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (I-2), (I′), (Ia), (Ib), (Ic), (Id), (Ie), (If), or (Ig), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (I-2), (I′), (Ia), (Ib), (Ic), (Id), (Ie), (If), or (Ig), or a pharmaceutically acceptable salt or solvate thereof, wherein R⁵ is selected from the group consisting of

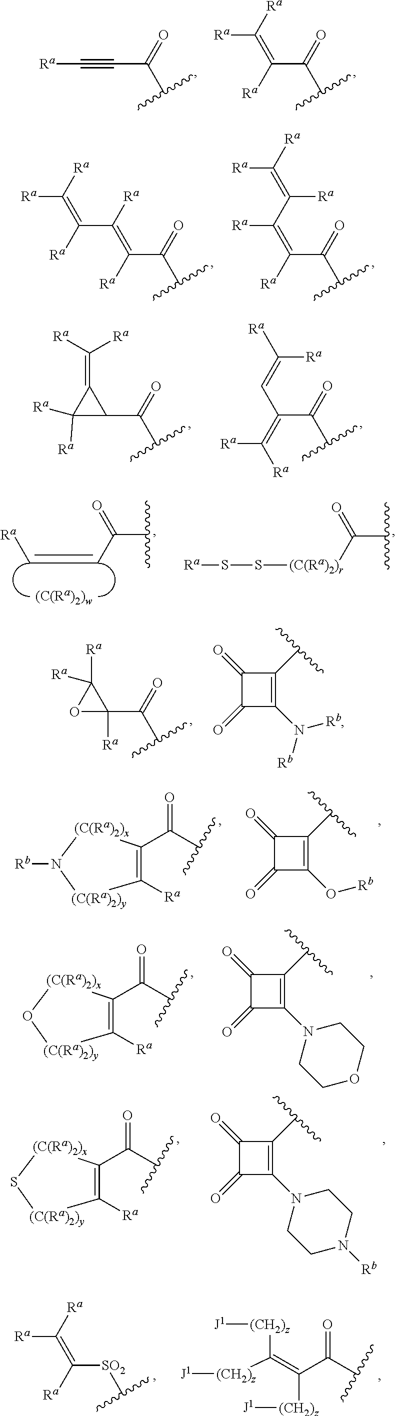

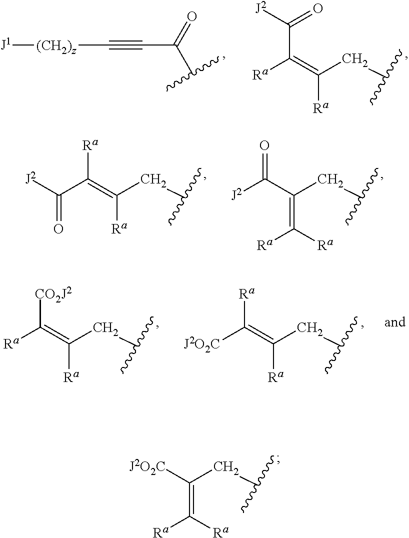

where each R^{a} is independently hydrogen, C₁₋₆alkyl, carboxy, C₁₋₆carboalkoxy, phenyl, C₂₋₇carboalkyl, R^{c}—(C(R^{b})₂)_{z}, R^{c}—(C(R^{b})₂)_{w}-M-(C(R^{b})₂)—, (R^{c})(R^{b})CH-M-(C(R^{b})₂)—, or Het-J³-(C(R⁴)₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

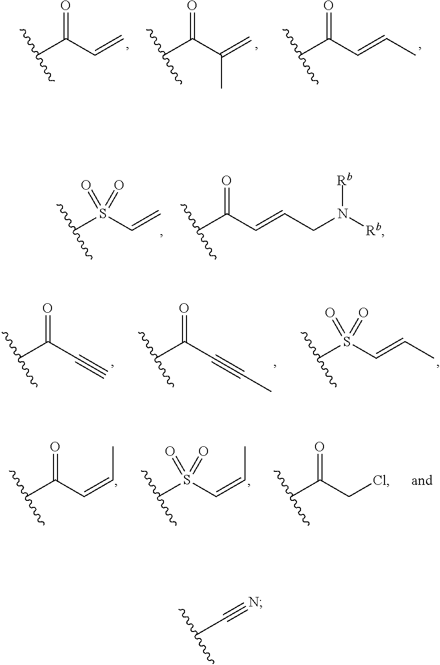

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In some embodiments is a compound of Formula (I″-1), or a pharmaceutically acceptable salt or solvate thereof:

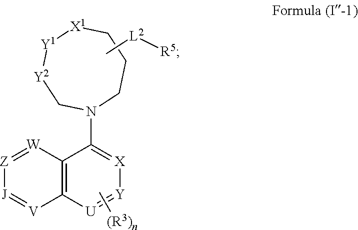

wherein:
X is C or N;
X¹ is selected from C(R⁴)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₉alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

In another aspect, the disclosure provides a compound of Formula (I″-2), or a pharmaceutically acceptable salt or solvate thereof:

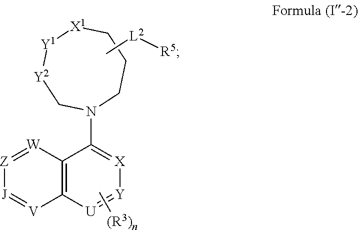

wherein:
X is C or N;
X¹ is selected from C(R⁴)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

In some embodiments is a compound of Formula (I″-1) or (I″-2), or a pharmaceutically acceptable salt or solvate thereof, wherein Y² is a bond. In some embodiments is a compound of Formula (I″-1) or (I″-2), or a pharmaceutically acceptable salt or solvate thereof, wherein Y² is CH₂. In some embodiments is a compound of Formula (I″-1) or (I″-2), or a pharmaceutically acceptable salt or solvate thereof, wherein Y¹ is CH₂. In some embodiments is a compound of Formula (I″-1) or (I″-2), or a pharmaceutically acceptable salt or solvate thereof, wherein X¹ is N(H). In some embodiments is a compound of Formula (I″-1) or (I″-2), or a pharmaceutically acceptable salt or solvate thereof, wherein X¹ is N(R⁴). In some embodiments is a compound of Formula (I″-1) or (I″-2), or a pharmaceutically acceptable salt or solvate thereof, wherein X¹ is N(-L²-R⁵). In some embodiments is a compound of Formula (I″-1) or (I″-2), or a pharmaceutically acceptable salt or solvate thereof, wherein X¹ is O. In some embodiments is a compound of Formula (I″-1) or (I″-2), or a pharmaceutically acceptable salt or solvate thereof, wherein X¹ is S. In some embodiments is a compound of Formula (I″-1) or (I″-2), or a pharmaceutically acceptable salt or solvate thereof, wherein X¹ is S(O)₂. In some embodiments is a compound of Formula (I″-1) or (I″-2), or a pharmaceutically acceptable salt or solvate thereof, wherein X¹ is CH₂. In some embodiments is a compound of Formula (I″-1) or (I″-2), or a pharmaceutically acceptable salt or solvate thereof, wherein X¹ is C(H)(R⁴). In some embodiments is a compound of Formula (I″-1) or (I″-2), or a pharmaceutically acceptable salt or solvate thereof, wherein X¹ is C(R⁴)₂. In some embodiments is a compound of Formula (I″-1) or (I″-2), or a pharmaceutically acceptable salt or solvate thereof, wherein X¹ is C(H)(-L²-R⁵).

In some embodiments is a compound of Formula (I″-1) or (I″-2), or a pharmaceutically acceptable salt or solvate thereof, wherein V is C(R¹⁶). In some embodiments is a compound of Formula (I″-1) or (I″-2), or a pharmaceutically acceptable salt or solvate thereof, wherein V is C(H). In some embodiments is a compound of Formula (I″-1) or (I″-2), or a pharmaceutically acceptable salt or solvate thereof, wherein V is N.

In some embodiments is a compound of Formula (I″-1) or (I″-2), or a pharmaceutically acceptable salt or solvate thereof, wherein W is C(R¹⁸). In some embodiments is a compound of Formula (I″-1) or (I″-2), or a pharmaceutically acceptable salt or solvate thereof, wherein W is C(H). In some embodiments is a compound of Formula (I″-1) or (I″-2), or a pharmaceutically acceptable salt or solvate thereof, wherein W is N.

In some embodiments is a compound of Formula (I″-1) or (I″-2), or a pharmaceutically acceptable salt or solvate thereof, wherein Z is C(R⁸). In some embodiments is a compound of Formula (I″-1) or (I″-2), or a pharmaceutically acceptable salt or solvate thereof, wherein Z is C(H). In some embodiments is a compound of Formula (I″-1) or (I″-2), or a pharmaceutically acceptable salt or solvate thereof, wherein Z is N.

In some embodiments is a compound of Formula (I″-1) or (I″-2), or a pharmaceutically acceptable salt or solvate thereof, wherein Y is C(R⁷). In some embodiments is a compound of Formula (I″-1) or (I″-2), or a pharmaceutically acceptable salt or solvate thereof, wherein R⁷ is selected from C₃₋₆cycloalkyl, C₂₋₉heterocycloalkyl, C₆₋₁₀aryl, C₁₋₉heteroaryl, —OR¹², —SR¹², and —N(R¹²)(R¹⁵), wherein C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (I″-1) or (I″-2), or a pharmaceutically acceptable salt or solvate thereof, wherein R⁷ is selected from —OR¹² and —SR¹². In some embodiments is a compound of Formula (I″-1) or (I″-2), or a pharmaceutically acceptable salt or solvate thereof, wherein R⁷ is C₁₋₆alkyl substituted with one, two, or three R^{20b}. In some embodiments is a compound of Formula (I″-1) or (I″-2), or a pharmaceutically acceptable salt or solvate thereof, wherein R⁷ is —OR¹².

In some embodiments is a compound of Formula (I″-1) or (I″-2), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (I″-1) or (I″-2), or a pharmaceutically acceptable salt or solvate thereof, wherein R¹² is C₁₋₆alkyl optionally substituted with one, two, or three R^{20d}. In some embodiments is a compound of Formula (I″-1) or (I″-2), or a pharmaceutically acceptable salt or solvate thereof, wherein R¹² is C₂₋₉heterocycloalkyl optionally substituted with one, two, or three R^{20d}. In some embodiments is a compound of Formula (I″-1) or (I″-2), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (I″-1) or (I″-2), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (I″-1) or (I″-2), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (I″-1) or (I″-2), or a pharmaceutically acceptable salt or solvate thereof, wherein R⁷ is selected from

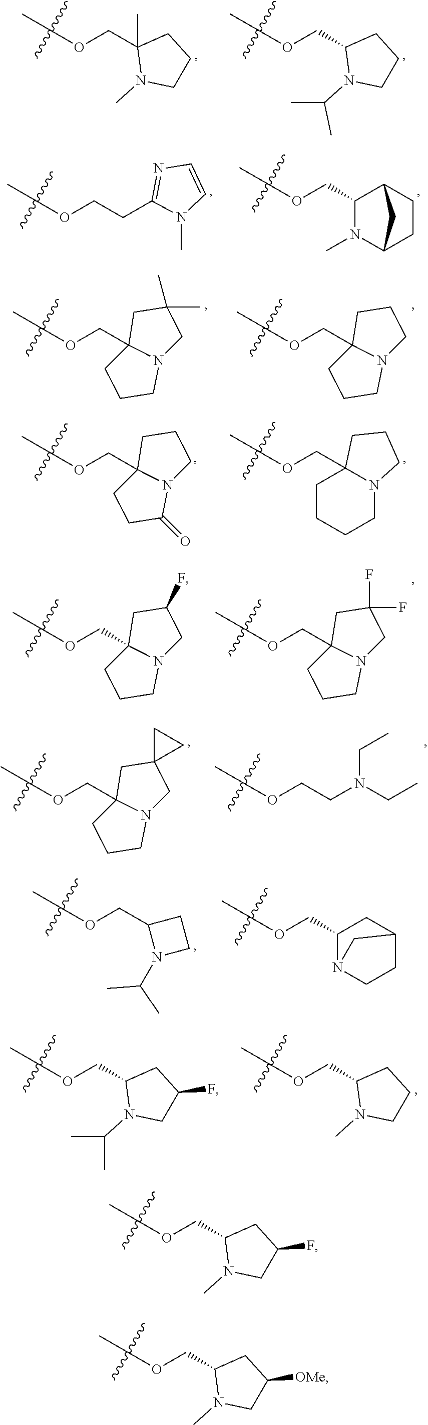

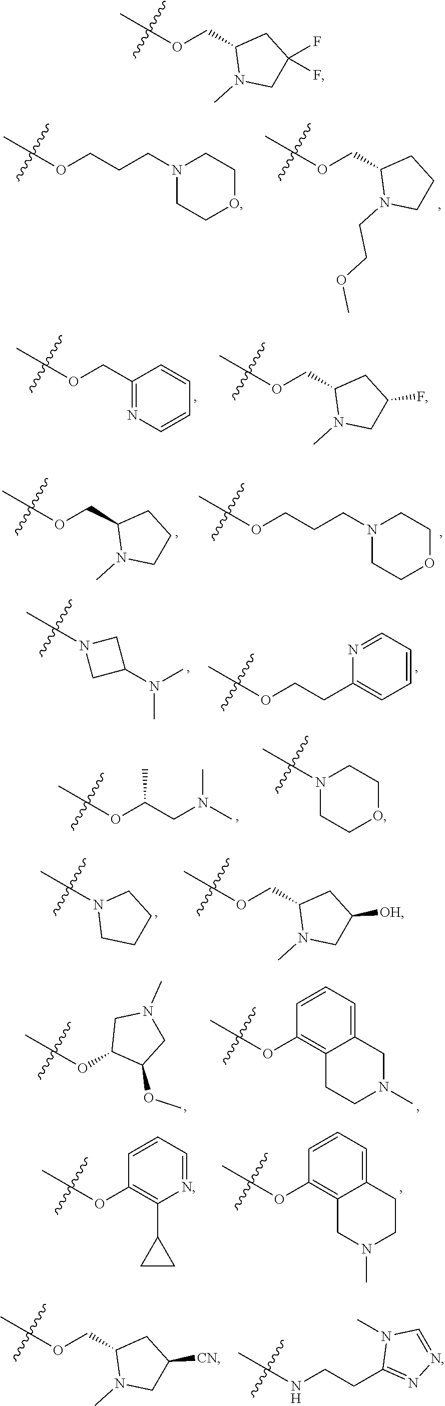

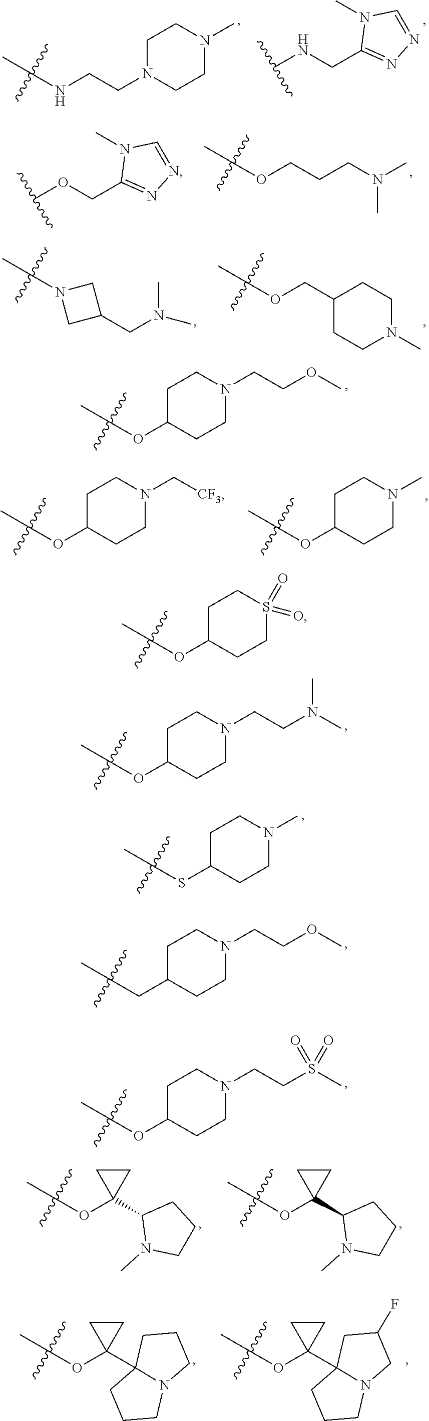

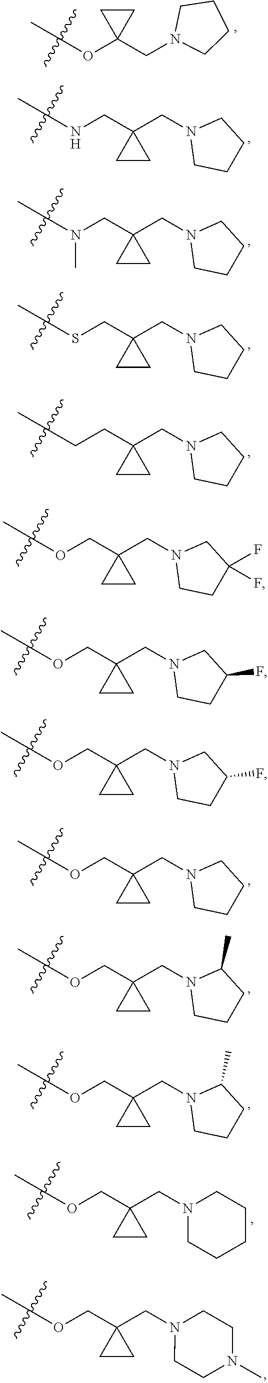

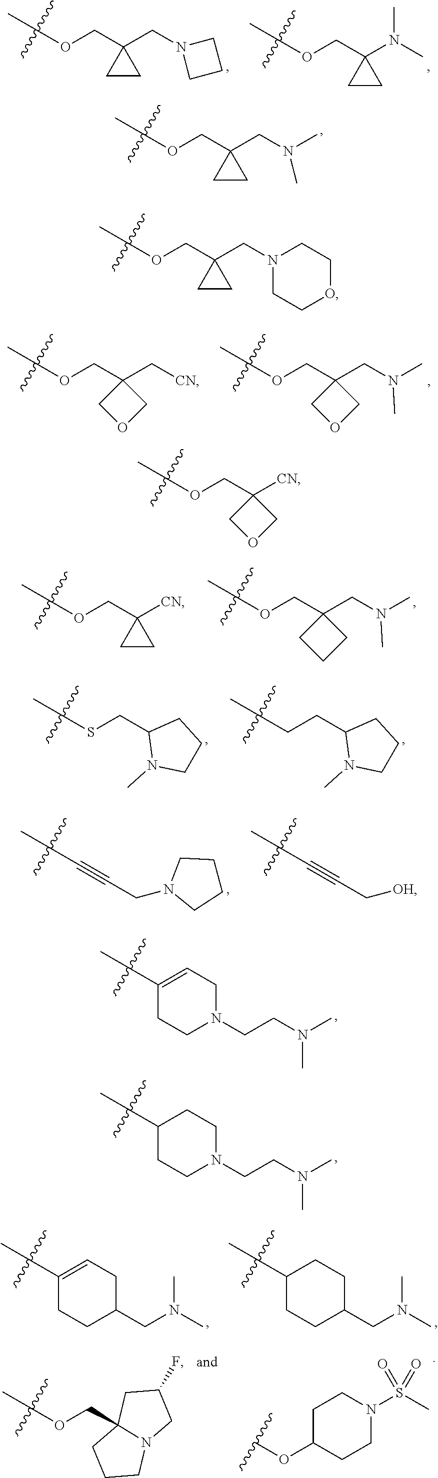

In some embodiments is a compound of Formula (I″-1) or (I″-2), or a pharmaceutically acceptable salt or solvate thereof, wherein L¹ is a bond. In some embodiments is a compound of Formula (I″-1) or (I″-2), or a pharmaceutically acceptable salt or solvate thereof, wherein L¹ is O.

In some embodiments is a compound of Formula (I″-1) or (I″-2), or a pharmaceutically acceptable salt or solvate thereof, wherein R¹⁹ is C₁₋₉heteroaryl optionally substituted with one, two, or three R²⁰ⁱ. In some embodiments is a compound of Formula (I″-1) or (I″-2), or a pharmaceutically acceptable salt or solvate thereof, wherein R¹⁹ is C₆₋₁₀aryl optionally substituted with one, two, or three R²⁰ⁱ.

In some embodiments is a compound of Formula (I″-1) or (I″-2), or a pharmaceutically acceptable salt or solvate thereof, wherein L² is selected from a bond, C₁-C₆alkyl, and —C(O)—. In some embodiments is a compound of Formula (I″-1) or (I″-2), or a pharmaceutically acceptable salt or solvate thereof, wherein L² is a bond. In some embodiments is a compound of Formula (I″-1) or (I″-2), or a pharmaceutically acceptable salt or solvate thereof, wherein L² is, C₁-C₆alkyl.

In some embodiments is a compound of Formula (I″-1)), or a pharmaceutically acceptable salt or solvate thereof, wherein R⁵ is hydrogen. In some embodiments is a compound of Formula (I″-1), or a pharmaceutically acceptable salt or solvate thereof, wherein R⁵ is a group other than an electrophilic moiety capable of forming a covalent bond with the cysteine residue at position 12 of a KRAS protein. In some embodiments is a compound of Formula (I″-1), or a pharmaceutically acceptable salt or solvate thereof, wherein R⁵ is —H, —NH₂, —OH, —NH(C₁₋₆ alkyl),

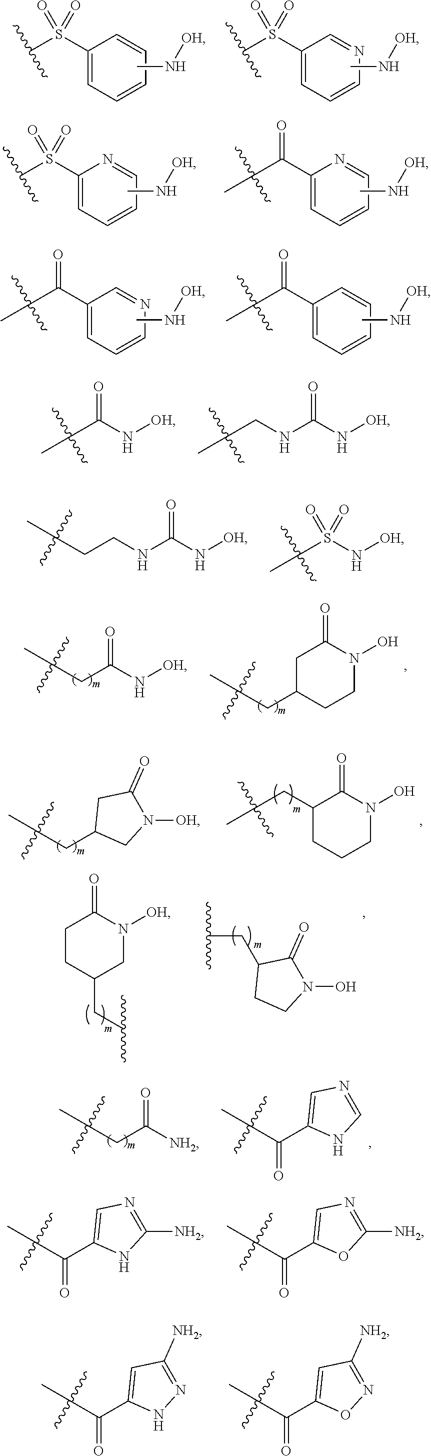

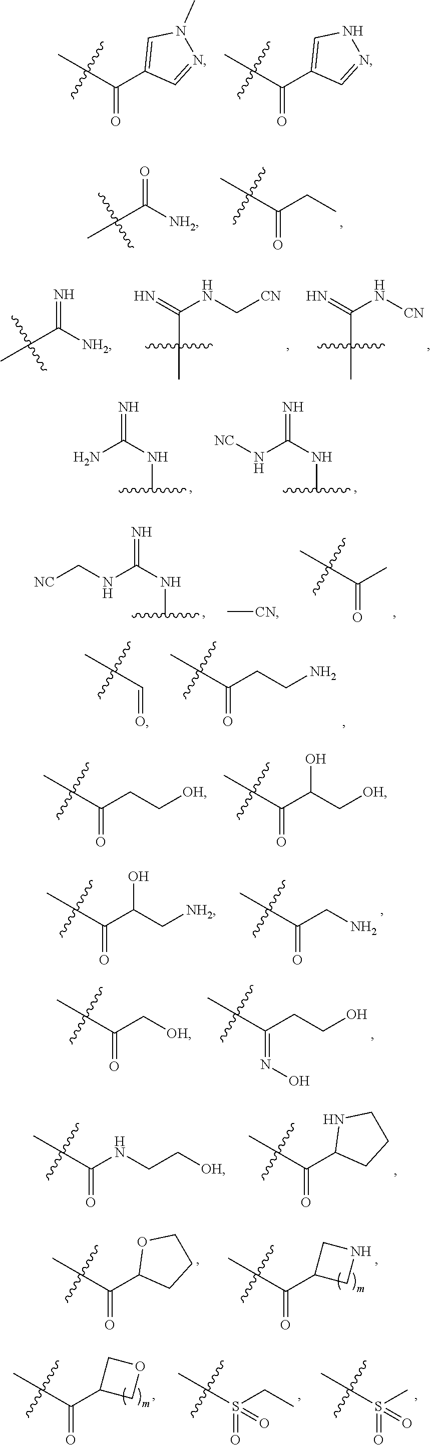

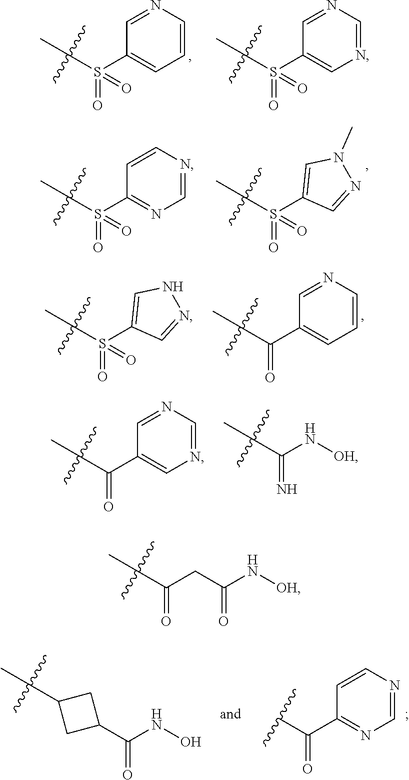

and m, when present, is 0, 1, 2, or 3.

In some embodiments of the subject compound of Formula (I″-1), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(—NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (I″-1), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (I″-1), or a pharmaceutically acceptable salt or solvate thereof, R⁵ is halogen. In further embodiments of the subject compound of Formula (I″-1), or a pharmaceutically acceptable salt or solvate thereof, R⁵ is —CN. In embodiments of the subject compound of Formula (I″-1), or a pharmaceutically acceptable salt or solvate thereof, R⁵ is —OH. In some embodiments of the subject compound of Formula (I″-1), or a pharmaceutically acceptable salt or solvate thereof, R⁵ is —NH₂. In further embodiments of the subject compound of Formula (I″-1), or a pharmaceutically acceptable salt or solvate thereof, R⁵ is —C(O)OH. In select embodiments of the subject compound of Formula (I″-1), or a pharmaceutically acceptable salt or solvate thereof, R⁵ is —OC(O)NH₂. In embodiments of the subject compound of Formula (I″-1), or a pharmaceutically acceptable salt or solvate thereof, R⁵ is —NHC(O)NH₂. In embodiments of the subject compound of Formula (I″-1), or a pharmaceutically acceptable salt or solvate thereof, R⁵ is —NHC(O)OH. In some embodiments of the subject compound of Formula (I″-1), or a pharmaceutically acceptable salt or solvate thereof, R⁵ is —NHS(O)₂H. In embodiments of the subject compound of Formula (I″-1), or a pharmaceutically acceptable salt or solvate thereof, R⁵ is —C(O)NH₂. In some embodiments of the subject compound of Formula (I″-1), or a pharmaceutically acceptable salt or solvate thereof, R⁵ is hydrogen. In embodiments of the subject compound of Formula (I″-1), or a pharmaceutically acceptable salt or solvate thereof, R⁵ is oxo. In further embodiments of the subject compound of Formula (I″-1), or a pharmaceutically acceptable salt or solvate thereof, R⁵ is C₁₋₆alkyl optionally substituted with one, two, or three R^{20a}. In select embodiments of the subject compound of Formula (I″-1), or a pharmaceutically acceptable salt or solvate thereof, R⁵ is C₃₋₆cycloalkyl optionally substituted with one, two, or three R^{20a}. In embodiments of the subject compound of Formula (I″-1), or a pharmaceutically acceptable salt or solvate thereof, R⁵ is C₂₋₉heterocycloalkyl optionally substituted with one, two, or three R^{20a}. In some embodiments of the subject compound of Formula (I″-1), or a pharmaceutically acceptable salt or solvate thereof, R⁵ is C₆₋₁₀aryl optionally substituted with one, two, or three R^{20a}. In further embodiments of the subject compound of Formula (I″-1), or a pharmaceutically acceptable salt or solvate thereof, R⁵ is C₁₋₉heteroaryl optionally substituted with one, two, or three R^{20a}. In select embodiments of the subject compound of Formula (I″-1), or a pharmaceutically acceptable salt or solvate thereof, R⁵ is —OR¹². In embodiments of the subject compound of Formula (I″-1), or a pharmaceutically acceptable salt or solvate thereof, R⁵ is —N(R¹²)(R¹³). In select embodiments of the subject compound of Formula (I″-1), or a pharmaceutically acceptable salt or solvate thereof, R⁵ is N(R¹⁴)S(O)₂R¹⁵. In select embodiments of the subject compound of Formula (I″-1), or a pharmaceutically acceptable salt or solvate thereof, R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (I″-2), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (I″-2), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (I″-2), or a pharmaceutically acceptable salt or solvate thereof, wherein R⁵ is selected from the group consisting of

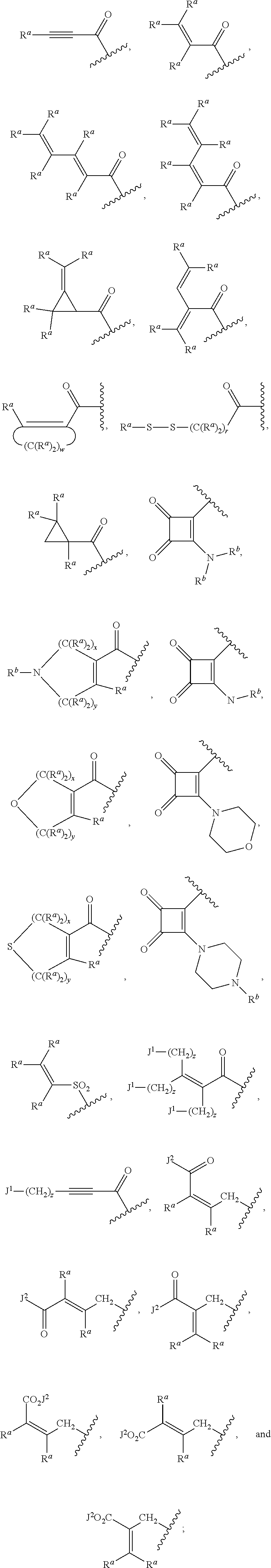

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{b}—(C(R^{b})₂)_{w}-M-(C(R^{b})₂), (R^{a})(R^{e})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{c} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

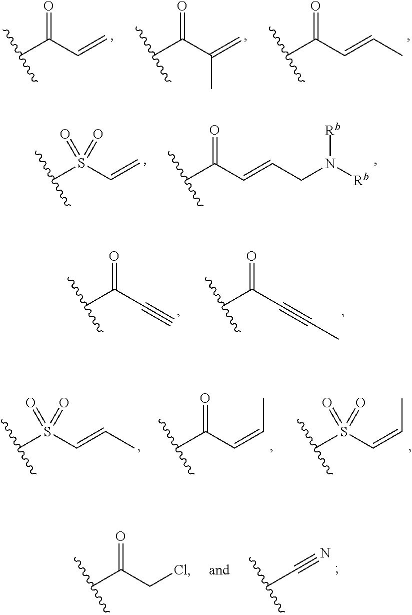

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In another aspect, the disclosure provides a compound of Formula (II-1), or a pharmaceutically acceptable salt or solvate thereof:

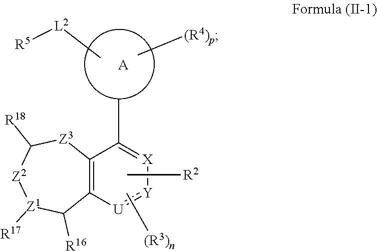

wherein:

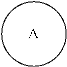

is a 7- or 8-membered monocyclic heterocycloalkyl ring;
X is C or N;
Y is C, S(O), S(O)₂, C(O), or N;
U is C, S(O), S(O)₂, C(O), or N;
W is Nor C(R¹⁸);
Z¹ is N or C(R⁶);
Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂-alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰%;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, R²⁰ⁱ, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

In another aspect, the disclosure provides a compound of Formula (II-2), or a pharmaceutically acceptable salt or solvate thereof:

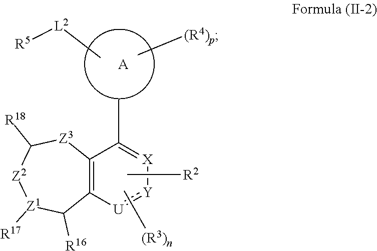

wherein:

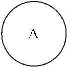

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸);
Z¹ is N or C(R⁶);
Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

In some embodiments is a compound of Formula (II-1) or (II-2) having the structure of Formula (IIa), or a pharmaceutically acceptable salt or solvate thereof:

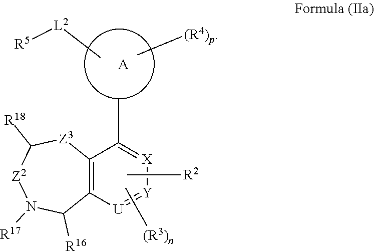

In some embodiments is a compound of Formula (II-1) or (II-2) having the structure of Formula (IIb), or a pharmaceutically acceptable salt or solvate thereof:

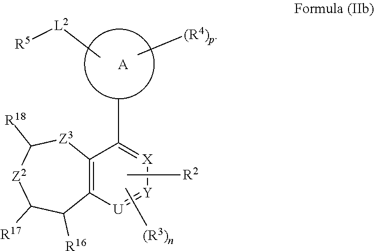

In some embodiments is a compound of Formula (II-1) or (II-2) having the structure of Formula (IIc′), or a pharmaceutically acceptable salt or solvate thereof:

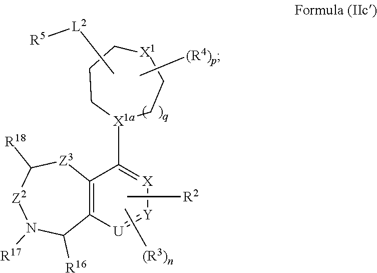

wherein X¹ is selected from C, N, O, S, S(O), and S(O)₂; X^{1a} is selected from N and C(H); and q is 1 or 2.

In some embodiments is a compound of Formula (II-1) or (II-2) having the structure of Formula (IIc), or a pharmaceutically acceptable salt or solvate thereof:

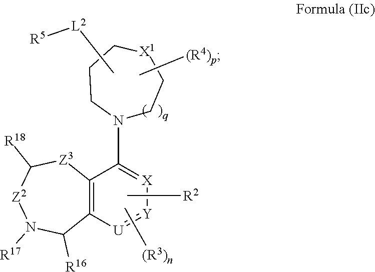

wherein X¹ is selected from C, N, O, S, S(O), and S(O)₂; and q is 1 or 2.

In some embodiments is a compound of Formula (II-1) or (II-2) having the structure of Formula (IId′), or a pharmaceutically acceptable salt or solvate thereof:

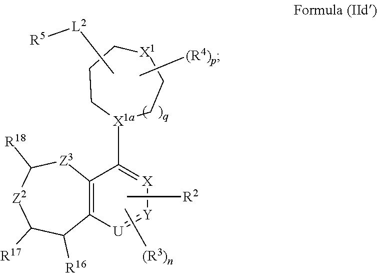

wherein X¹ is selected from C, N, O, S, S(O), and S(O)₂; X^{1a} is selected from N and C(H); and q is 1 or 2.

In some embodiments is a compound of Formula (II-1) or (II-2) having the structure of Formula (IId), or a pharmaceutically acceptable salt or solvate thereof:

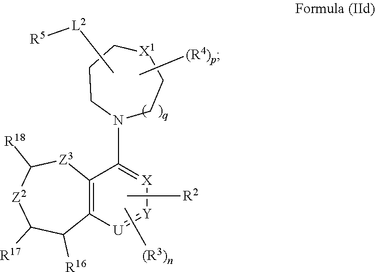

wherein X¹ is selected from C, N, O, S, S(O), and S(O)₂; and q is 1 or 2.

In some embodiments is a compound of Formula (II-1), (II-2), (IIa), (IIb), (IIc′), (IIc), (IId′), or (IId), or a pharmaceutically acceptable salt or solvate thereof, wherein X¹ is N(H). In some embodiments is a compound of Formula (II-1), (II-2), (IIa), (IIb), (IIc′), (IIc), (IId′), or (IId), or a pharmaceutically acceptable salt or solvate thereof, wherein X¹ is N(R⁴). In some embodiments is a compound of Formula (II-1), (II-2), (IIa), (IIb), (IIc′), (IIc), (IId′), or (IId), or a pharmaceutically acceptable salt or solvate thereof, wherein X¹ is N(-L²-R⁵). In some embodiments is a compound of Formula (II-1), (II-2), (IIa), (IIb), (IIc′), (IIc), (IId′), or (IId), or a pharmaceutically acceptable salt or solvate thereof, wherein X¹ is O. In some embodiments is a compound of Formula (II-1), (II-2), (IIa), (IIb), (IIc′), (IIc), (IId′), or (IId), or a pharmaceutically acceptable salt or solvate thereof, wherein X¹ is S. In some embodiments is a compound of Formula (II-1), (II-2), (IIa), (IIb), (IIc′), (IIc), (IId′), or (IId), or a pharmaceutically acceptable salt or solvate thereof, wherein X¹ is S(O)₂. In some embodiments is a compound of Formula (II-1), (II-2), (IIa), (IIb), (IIc′), (IIc), (IId′), or (IId), or a pharmaceutically acceptable salt or solvate thereof, wherein X¹ is CH₂. In some embodiments is a compound of Formula (II-1), (II-2), (IIa), (IIb), (IIc′), (IIc), (IId′), or (IId), or a pharmaceutically acceptable salt or solvate thereof, wherein X¹ is C(H)(R⁴). In some embodiments is a compound of Formula (II-1), (II-2), (IIa), (IIb), (IIc′), (IIc), (IId′), or (IId), or a pharmaceutically acceptable salt or solvate thereof, wherein X¹ is C(R⁴)₂. In some embodiments is a compound of Formula (II-1), (II-2), (IIa), (IIb), (IIc′), (IIc), (IId′), or (IId), or a pharmaceutically acceptable salt or solvate thereof, wherein X¹ is C(H)(-L²-R⁵).

In some embodiments is a compound of Formula (II-1), (II-2), (IIa), (IIb), (IIc′), (IIc), (IId′), or (IId), or a pharmaceutically acceptable salt or solvate thereof, wherein q is 1. In some embodiments is a compound of Formula (II-1), (II-2), (IIa), (IIb), (IIc′), (IIc), (IId′), or (IId), or a pharmaceutically acceptable salt or solvate thereof, wherein q is 2.

In some embodiments is a compound of Formula (II-1), (II-2), (IIa), (IIb), (IIc′), (IIc), (IId′), or (IId), or a pharmaceutically acceptable salt or solvate thereof, wherein Z³ is absent.

In some embodiments is a compound of Formula (II-1), (II-2), (IIa), (IIb), (IIc′), (IIc), (IId′), or (IId), or a pharmaceutically acceptable salt or solvate thereof, wherein Z² is C(R⁸)(R⁹).

In some embodiments is a compound of Formula (II-1), (II-2), (IIa), (IIb), (IIc′), (IIc), (IId′), or (IId), or a pharmaceutically acceptable salt or solvate thereof, wherein R⁹ is hydrogen.

In some embodiments is a compound of Formula (II-1), (II-2), (IIa), (IIb), (IIc′), (IIc), (IId′), or (IId), or a pharmaceutically acceptable salt or solvate thereof, wherein X is C. In some embodiments is a compound of Formula (II-1), (II-2), (IIa), (IIb), (IIc′), (IIc), (IId′), or (IId), or a pharmaceutically acceptable salt or solvate thereof, wherein X is N.

In some embodiments is a compound of Formula (II-1), (II-2), (IIa), (IIb), (IIc′), (IIc), (IId′), or (IId), or a pharmaceutically acceptable salt or solvate thereof, wherein Y is C. In some embodiments is a compound of Formula (II-1), (II-2), (IIa), (IIb), (IIc′), (IIc), (IId′), or (IId), or a pharmaceutically acceptable salt or solvate thereof, wherein Y is N. In some embodiments is a compound of Formula (II-1), (II-2), (IIa), (IIb), (IIc′), (IIc), (IId′), or (IId), or a pharmaceutically acceptable salt or solvate thereof, wherein Y is C(O).

In some embodiments is a compound of Formula (II-1), (II-2), (IIa), (IIb), (IIc′), (IIc), (IId′), or (IId), or a pharmaceutically acceptable salt or solvate thereof, wherein U is C. In some embodiments is a compound of Formula (II-1), (II-2), (IIa), (IIb), (IIc′), (IIc), (IId′), or (IId), or a pharmaceutically acceptable salt or solvate thereof, wherein U is N. In some embodiments is a compound of Formula (II-1), (II-2), (IIa), (IIb), (IIc′), (IIc), (IId′), or (IId), or a pharmaceutically acceptable salt or solvate thereof, wherein U is C(O).

In some embodiments is a compound of Formula (II-1) or (II-2) having the structure of Formula (IIe), or a pharmaceutically acceptable salt or solvate thereof:

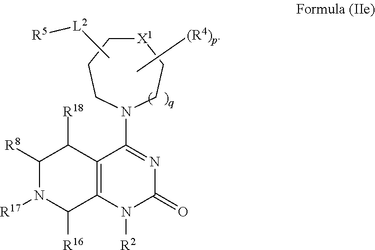

In some embodiments is a compound of Formula (II-1) or (II-2) having the structure of Formula (IIf), or a pharmaceutically acceptable salt or solvate thereof:

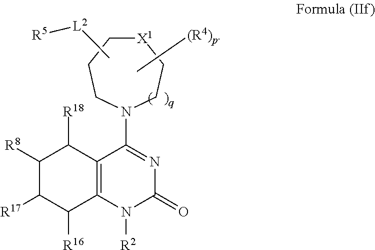

In some embodiments is a compound of Formula (II-1) or (II-2) having the structure of Formula (IIg), or a pharmaceutically acceptable salt or solvate thereof:

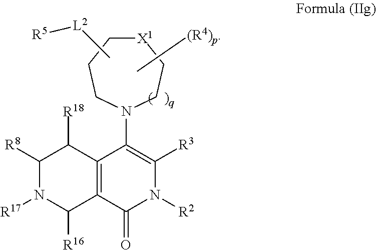

In some embodiments is a compound of Formula (II-1) or (II-2) having the structure of Formula (IIh), or a pharmaceutically acceptable salt or solvate thereof:

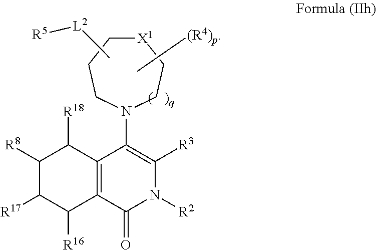

In some embodiments is a compound of Formula (II-1) or (II-2) having the structure of Formula (IIi), or a pharmaceutically acceptable salt or solvate thereof:

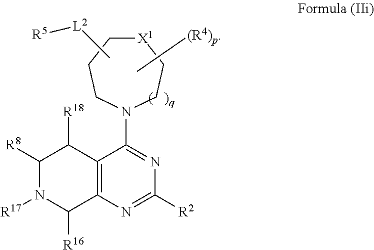

In some embodiments is a compound of Formula (II-1) or (II-2) having the structure of Formula (IIj), or a pharmaceutically acceptable salt or solvate thereof:

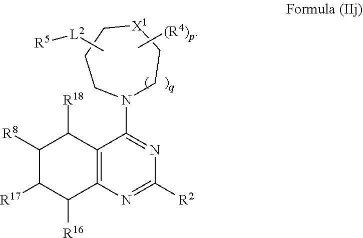

In some embodiments is a compound of Formula (II-1) or (II-2) having the structure of Formula (II), or a pharmaceutically acceptable salt or solvate thereof:

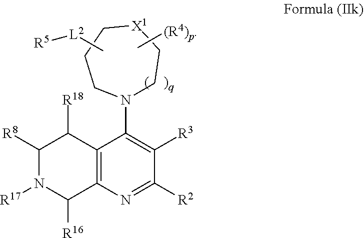

In some embodiments is a compound of Formula (II-1) or (II-2) having the structure of Formula (IIm), or a pharmaceutically acceptable salt or solvate thereof:

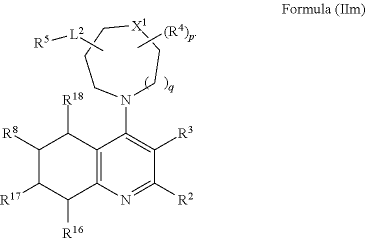

In some embodiments is a compound of Formula (IIe), (IIf), (IIg), (IIh), (IIi), (IIj), (IIk), or (IIm), or a pharmaceutically acceptable salt or solvate thereof, wherein X¹ is N(H). In some embodiments is a compound of Formula (IIe), (IIf), (IIg), (IIh), (IIi), (IIj), (IIk), or (IIm), or a pharmaceutically acceptable salt or solvate thereof, wherein X¹ is N(R⁴). In some embodiments is a compound of Formula (IIe), (IIf), (IIg), (IIh), (IIi), (IIj), (IIk), or (IIm), or a pharmaceutically acceptable salt or solvate thereof, wherein X¹ is N(-L²-R⁵). In some embodiments is a compound of Formula (IIe), (IIf), (IIg), (IIh), (IIi), (IIj), (IIk), or (IIm), or a pharmaceutically acceptable salt or solvate thereof, wherein X¹ is O. In some embodiments is a compound of Formula (IIe), (IIf), (IIg), (IIh), (IIi), (IIj), (IIk), or (IIm), or a pharmaceutically acceptable salt or solvate thereof, wherein X¹ is S. In some embodiments is a compound of Formula (IIe), (IIf), (IIg), (IIh), (IIi), (IIj), (IIk), or (IIm), or a pharmaceutically acceptable salt or solvate thereof, wherein X¹ is S(O)₂. In some embodiments is a compound of Formula (IIe), (IIf), (IIg), (IIh), (IIi), (IIj), (IIk), or (IIm), or a pharmaceutically acceptable salt or solvate thereof, wherein X¹ is CH₂. In some embodiments is a compound of Formula (IIe), (IIf), (IIg), (IIh), (IIi), (IIj), (IIk), or (IIm), a pharmaceutically acceptable salt or solvate thereof, wherein X¹ is C(H)(R⁴). In some embodiments is a compound of Formula (IIe), (IIf), (IIg), (IIh), (IIi), (IIj), (IIk), or (IIm), or a pharmaceutically acceptable salt or solvate thereof, wherein X¹ is C(R⁴)₂. In some embodiments is a compound of Formula (IIe), (IIf), (IIg), (IIh), (IIi), (IIj), (IIk), or (IIm), or a pharmaceutically acceptable salt or solvate thereof, wherein X¹ is C(H)(-L²-R⁵).

In some embodiments is a compound of Formula (IIe), (IIf), (IIg), (IIh), (IIi), (IIj), (IIk), or (IIm), or a pharmaceutically acceptable salt or solvate thereof, wherein q is 1. In some embodiments is a compound of Formula (IIe), (IIf), (IIg), (IIh), (IIi), (IIj), (IIk), or (IIm), or a pharmaceutically acceptable salt or solvate thereof, wherein q is 2.

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (II-1), (II-2), (IIa), (IIb), (IIc′), (IIc), (IId′), (IId), (Ile), (IIf), (IIg), (IIh), (IIi), (IIj), (IIk), or (IIm), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R² is —OR¹².

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹² is C₁₋₆alkyl optionally substituted with one, two, or three R^{20d}. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹² is C₂₋₉heterocycloalkyl optionally substituted with one, two, or three R^{20d}. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R² is selected from

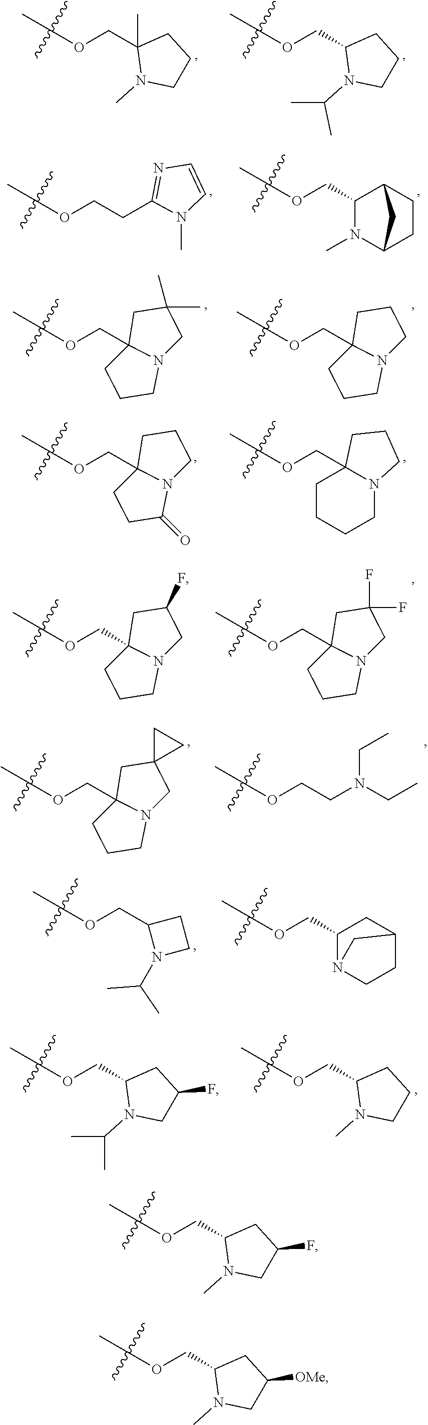

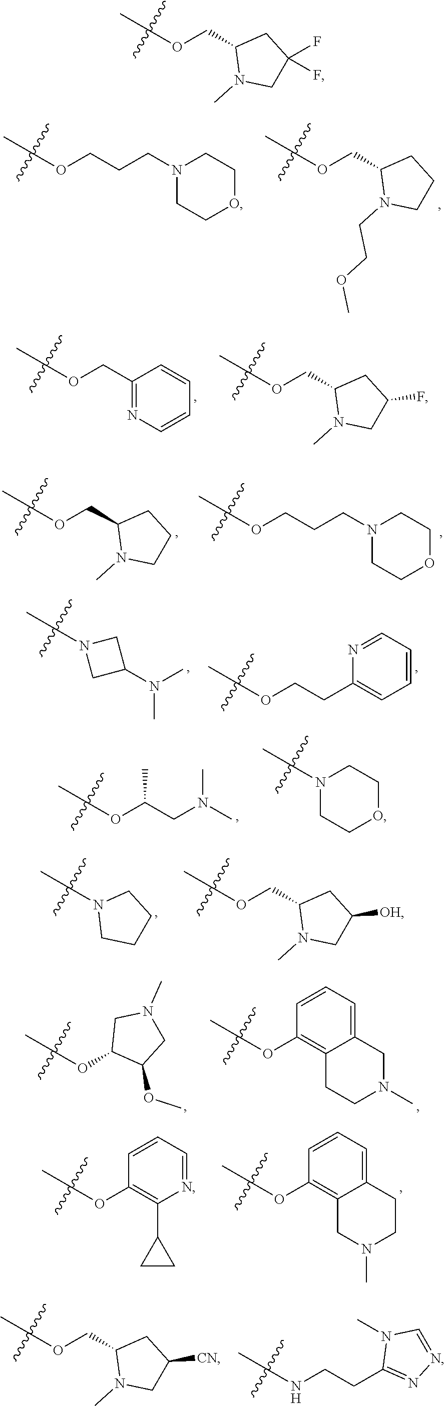

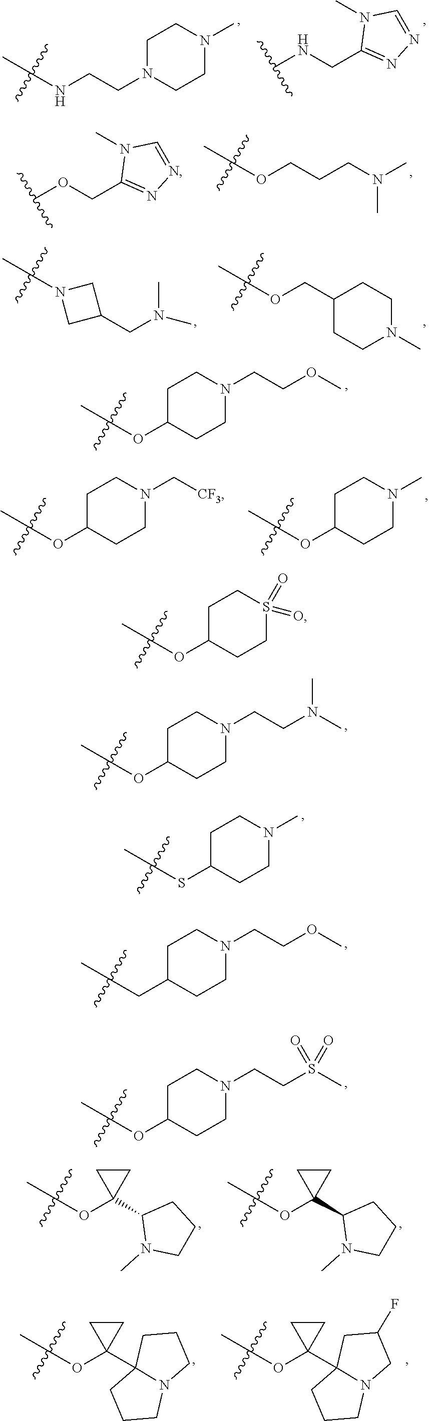

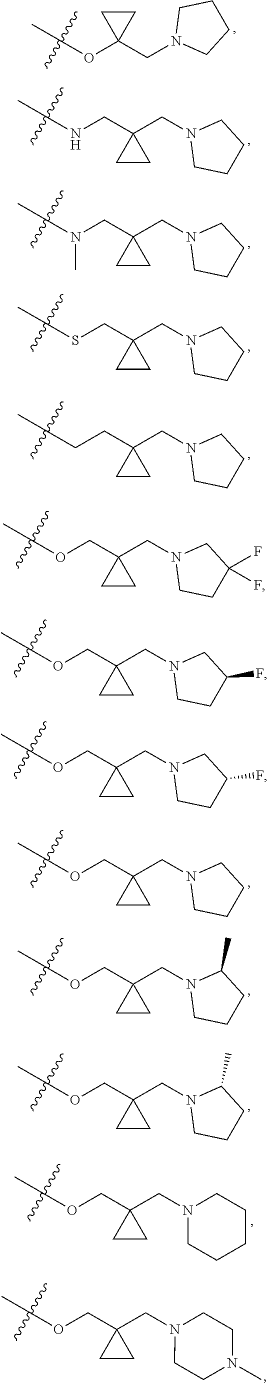

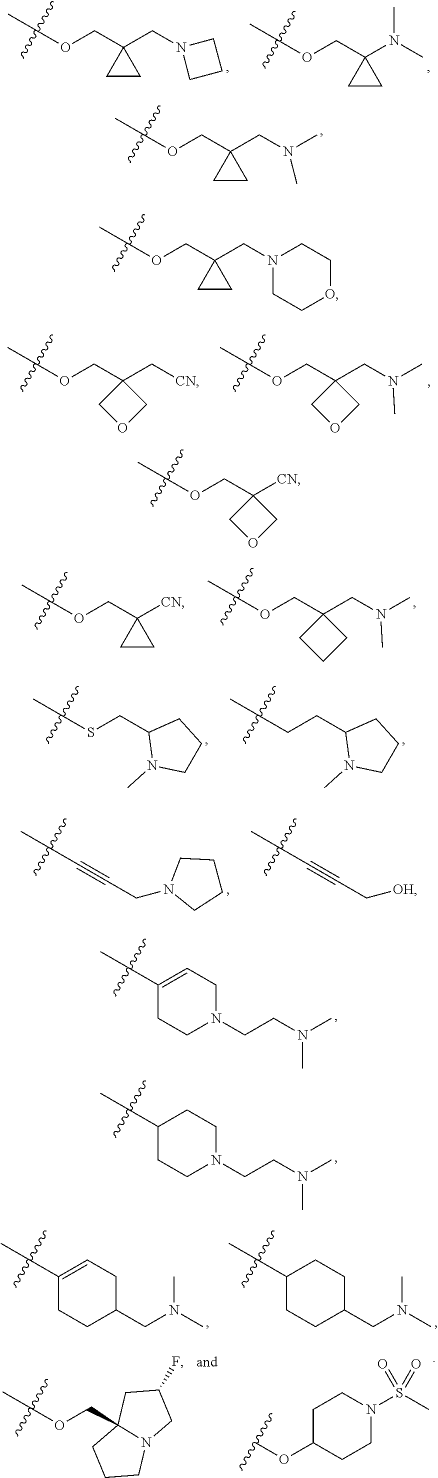

In some embodiments is a compound of Formula (II-1), (II-2), (IIa), (IIb), (IIc′), (IIc), (IId′), (IId), (Ile), (IIf), (IIg), (IIh), (IIi), (IIj), (IIk), or (IIm), or a pharmaceutically acceptable salt or solvate thereof, wherein L¹ is a bond. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein L¹ is O.

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹⁹ is C₁₋₉heteroaryl optionally substituted with one, two, or three R²⁰ⁱ. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R¹⁹ is C₆₋₁₀aryl optionally substituted with one, two, or three R²⁰ⁱ.

In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein L² is selected from a bond, C₁-C₆alkyl, and —C(O)—. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein L² is a bond. In some embodiments is a compound of Formula (II-1), (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein L² is, C₁-C₆alkyl.

In some embodiments is a compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is hydrogen. In some embodiments is a compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is a group other than an electrophilic moiety capable of forming a covalent bond with the cysteine residue at position 12 of a KRAS protein. In some embodiments is a compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is —H, —NH₂, —OH, —NH(C₁₋₆ alkyl),

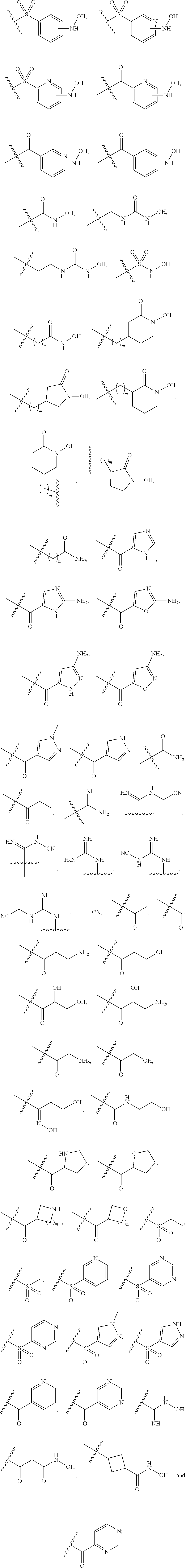

and m, when present, is 0, 1, 2, or 3.

In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is halogen. In further embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —CN. In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —OH. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —NH₂. In further embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —C(O)OH. In select embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —OC(O)NH₂. In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —NHC(O)NH₂. In embodiments of the subject compound of Formula (II-1), (IIa), (IIb), (IIc′), (IIc), (IId′), (IId), (Ile), (IIf), (IIg), (IIh), (IIi), (IIj), (IIk), or (IIm), or a pharmaceutically acceptable salt or solvate thereof, R⁵ is —NHC(O)OH. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —NHS(O)₂H. In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —C(O)NH₂. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is hydrogen. In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is oxo. In further embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is C₁₋₆alkyl optionally substituted with one, two, or three R^{20a}. In select embodiments of the subject compound of Formula (II-1), (IIa), (IIb), (IIc′), (IIc), (IId′), (IId), (Ile), (IIf), (IIg), (IIh), (IIi), (IIj), (IIk), or (IIm), or a pharmaceutically acceptable salt or solvate thereof, R⁵ is C₃₋₆cycloalkyl optionally substituted with one, two, or three R^{20a}. In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is C₂₋₉heterocycloalkyl optionally substituted with one, two, or three R^{20a}. In some embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is C₆₋₁₀aryl optionally substituted with one, two, or three R^{20a}. In further embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is C₁₋₉heteroaryl optionally substituted with one, two, or three R^{20a}. In select embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —OR¹². In embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is —N(R¹²)(R¹³). In select embodiments of the subject compound of Formula (II-1), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, R⁵ is N(R¹⁴)S(O)₂R¹⁵. In select embodiments of the subject compound of Formula (II-1), (IIa), (IIb), (IIc′), (IIc), (IId′), (IId), (Ile), (IIf), (IIg), (IIh), (IIi), (IIj), (IIk), or (IIm), or a pharmaceutically acceptable salt or solvate thereof, R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (II-2), (IIa), (IIb), (IIc′), (IIc), (IId′), (IId), (IIe), (IIf), (IIg), (IIh), (IIi), (IIj), (IIk), or (IIm), or a pharmaceutically acceptable salt or solvate thereof, wherein R⁵ is selected from the group consisting of

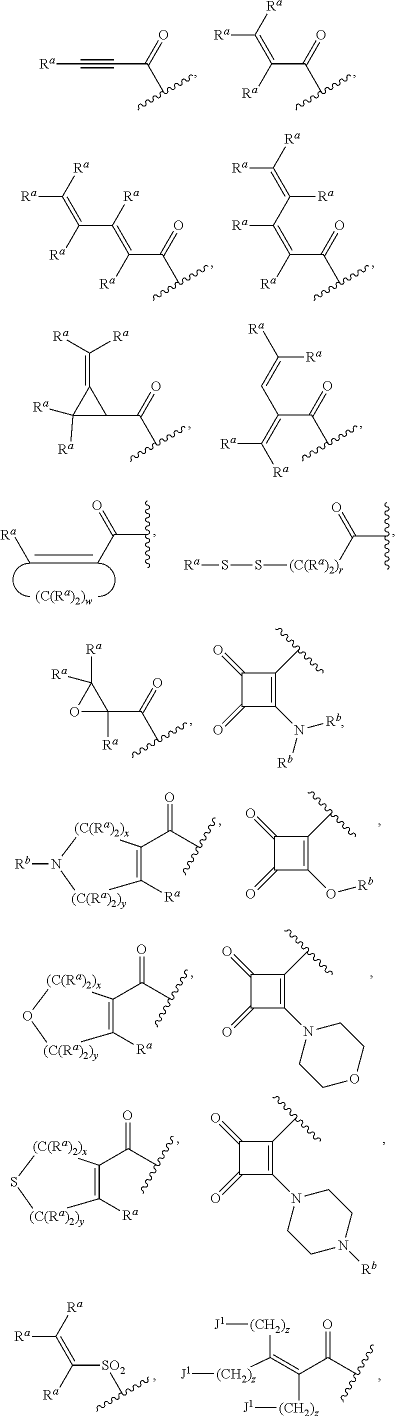

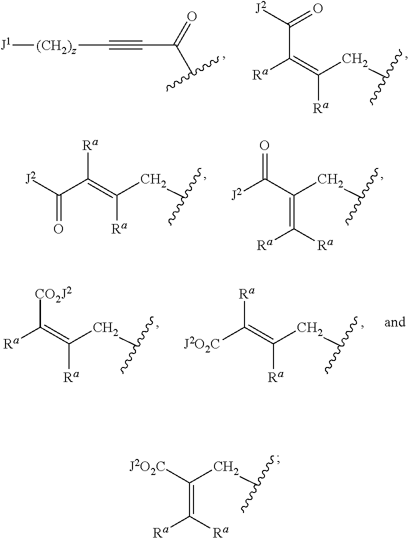

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-(C(R^{b})₂)ᵣ—, (R^{d})(R^{e})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{e} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

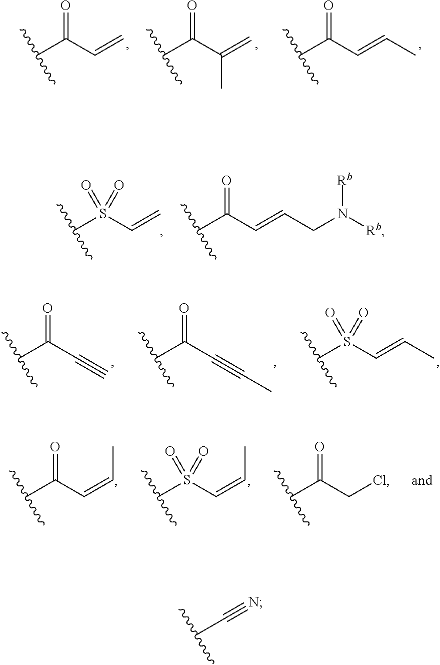

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In another aspect, the disclosure provides a compound of Formula (IIIa-1)-(IIIi-1), or a pharmaceutically acceptable salt or solvate thereof:

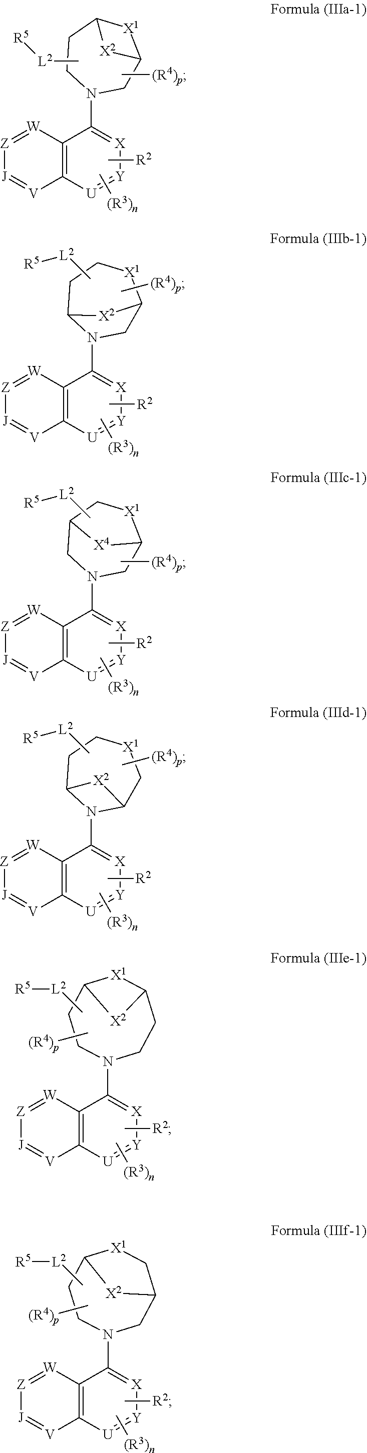

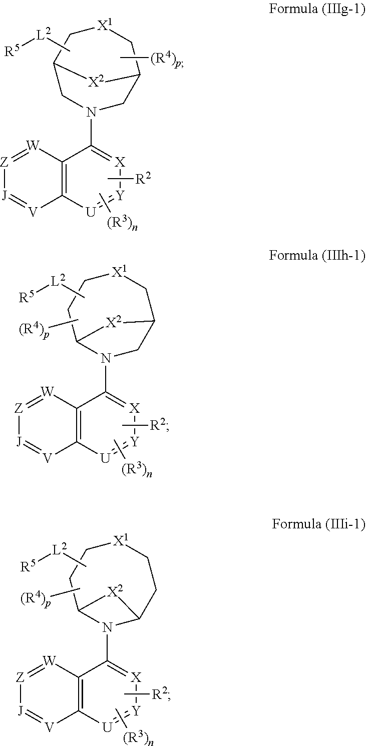

wherein:
X is C or N;
X¹ is selected from C(R⁴)(R⁶), N(R⁴), N(R⁶), O, S, S(O), and S(O);
X² is selected from X³, —CH₂—, —X³CH₂—, —CH₂X³—, —CH₂CH₂—, —C(H)(R⁴)—, —C(H)(R⁴)—, —XC(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴),X³—, —((H)C(R⁴)₂—, —C(R⁴)₂—, C(H)—, and —C(R⁴)₂C(R⁴)₂—;
X³ is selected from N(R¹), O, S, S(O), and S(O)₂;
X⁴ is selected from X⁵, —CH₂—, —X⁵CH₂—, —CH₂X³—, —CH₂CH₂—, —C(H)(R⁴)—, —C(H)(R⁴)—, —X⁵—C (H)(R⁴)—, —C(H)(R⁴)X³—, —C(H)(R⁴)CH₂—, —C(H)(R⁴)C(H)(R⁴)—, —C(R⁴)₂—, —X⁵—, —C(R⁴)₂—, —C(R⁴)₂X⁵—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-2)-(IIIi-2), or a pharmaceutically acceptable salt or solvate thereof:

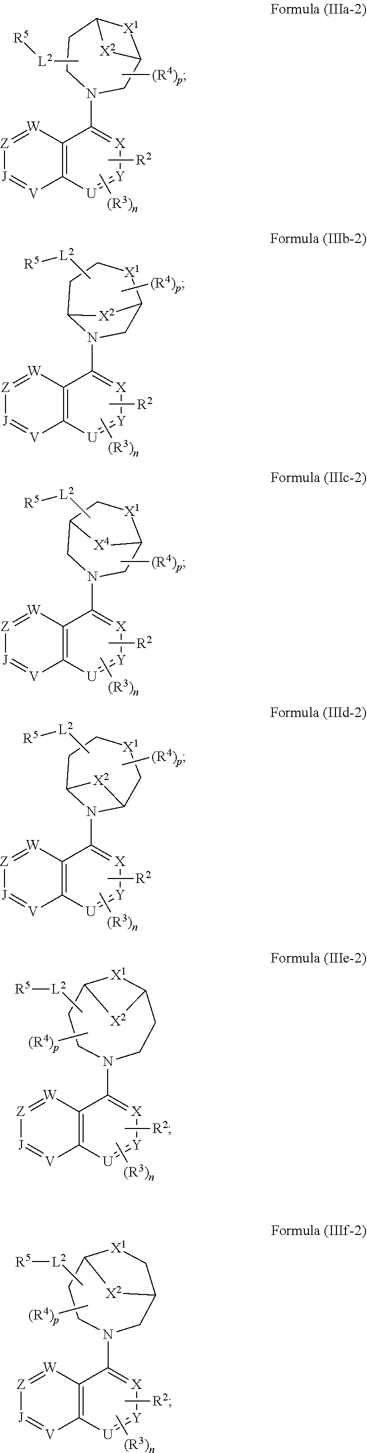

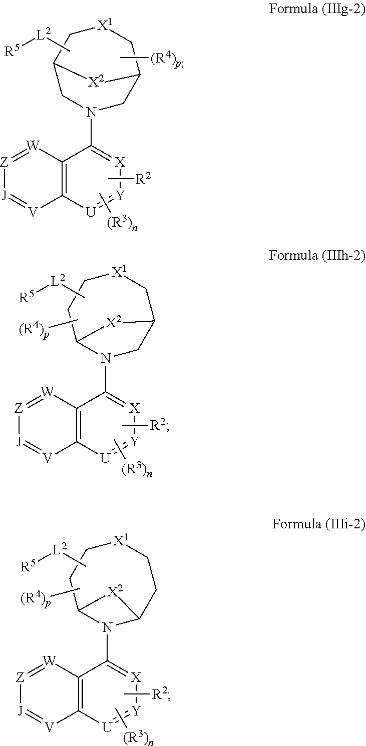

wherein:
X is C or N;
X¹ is selected from C(R⁴)(R⁶), N(R⁴), N(R⁶), O, S, S(O), and S(O)₂;
X² is selected from X³, —CH₂, —X³CH₂—, —CH₂X³—, —CH₂CH₂—, —C(H)(R⁴)—, —C(H)(R⁴)—, —X⁵C(H)(R⁴)—, —C(H)(R⁴)X³—, —C(H)(R⁴)CH₂—, —C(H)(R⁴)₂—, —C(H)(R⁴)—, —C(R⁴)₂—, —X³C(R⁴)₂—, —C(R⁴)₂—X³—, —C(H)C(R⁴)₂—, —C(R⁴)₂—C(H)—, and —C(R⁴)₂—C(R⁴)₂—;
X³ is selected from N(R¹), O, S, S(O), and S(O)₂;
X⁴ is selected from X³, —CH₂, —X³CH₂, —CH₂X³—, —CH₂CH₂, —C(H)(R⁴)—, —C(H)(R⁴)—, —X³C(H)(R⁴)—, —C(H)(R⁴)X³—, —C(H)(R⁴)CH₂—, —C(H)(R⁴)₂—, C(H)(R⁴)₂—, —C(R⁴)₂—, —XC(R⁴)₂—, —C(R⁴)₂—X³—, —C(H)C(R⁴)₂—, —C(R⁴)₂—C(H)—, and —C(R⁴)₂—C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}, R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound having the structure of Formula (IIIa-1) or (IIIa-2), or a pharmaceutically acceptable salt or solvate thereof:

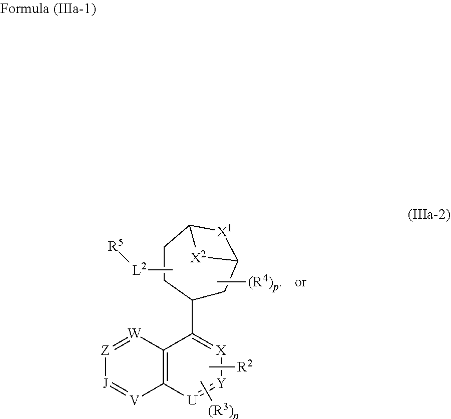

In some embodiments is a compound having the structure of Formula (IIIb-1) or (IIIb-2), or a pharmaceutically acceptable salt or solvate thereof:

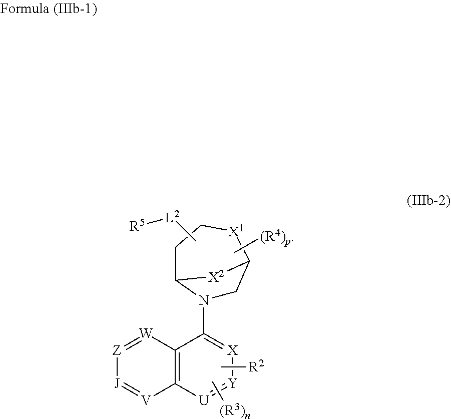

In some embodiments is a compound having the structure of Formula (IIId-1) or (IIId-2), or a pharmaceutically acceptable salt or solvate thereof:

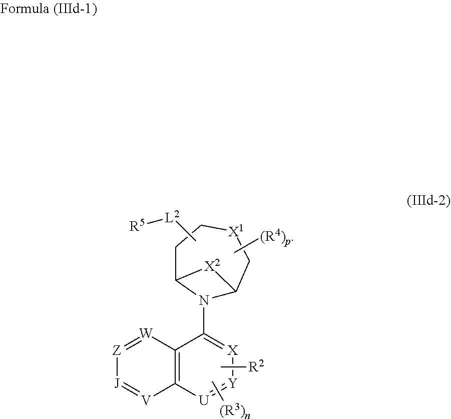

In some embodiments is a compound having the structure of Formula (IIIe-1) or (IIIc-2), or a pharmaceutically acceptable salt or solvate thereof:

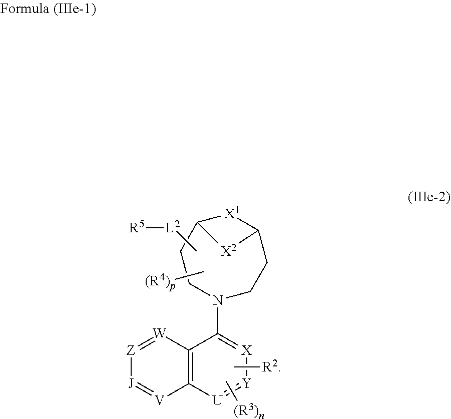

In some embodiments is a compound having the structure of Formula (IIIf-1) or (IIIf-2), or a pharmaceutically acceptable salt or solvate thereof:

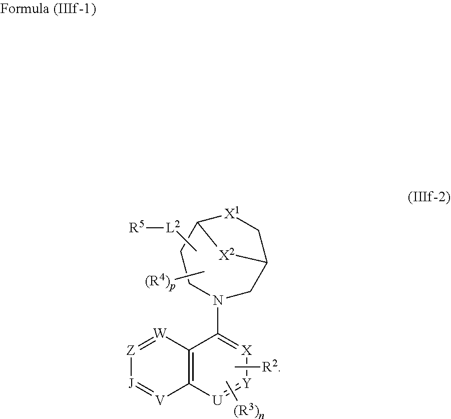

In some embodiments is a compound having the structure of Formula (IIIg-1) or (IIIg-2), or a pharmaceutically acceptable salt or solvate thereof:

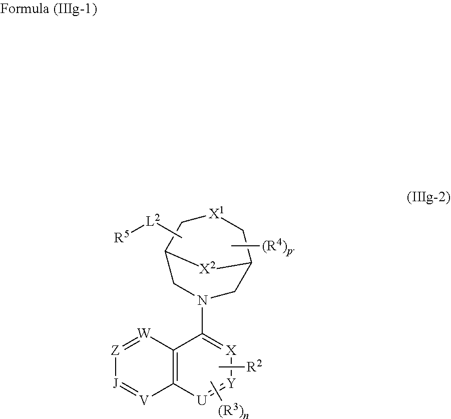

In some embodiments is a compound having the structure of Formula (IIIh-1) or (IIIh-2), or a pharmaceutically acceptable salt or solvate thereof:

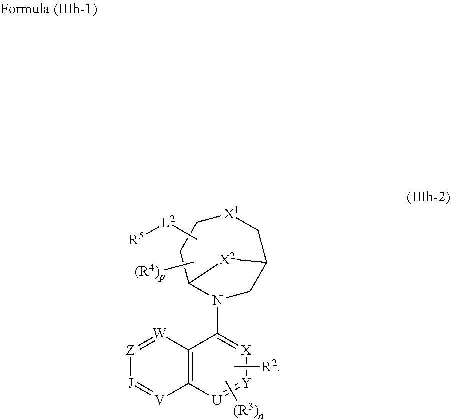

In some embodiments is a compound having the structure of Formula (IIIi-1) or (IIIi-2), or a pharmaceutically acceptable salt or solvate thereof:

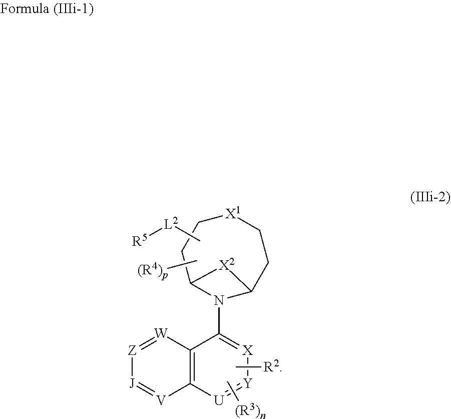

In some embodiments is a compound having the structure of Formula (IIIc-1) or (IIIc-2), or a pharmaceutically acceptable salt or solvate thereof:

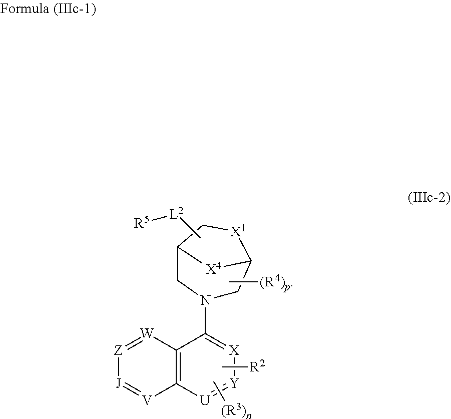

In another aspect, the disclosure provides a compound of Formula (IIIa-3)-(IIIi-3), or a pharmaceutically acceptable salt or solvate thereof:

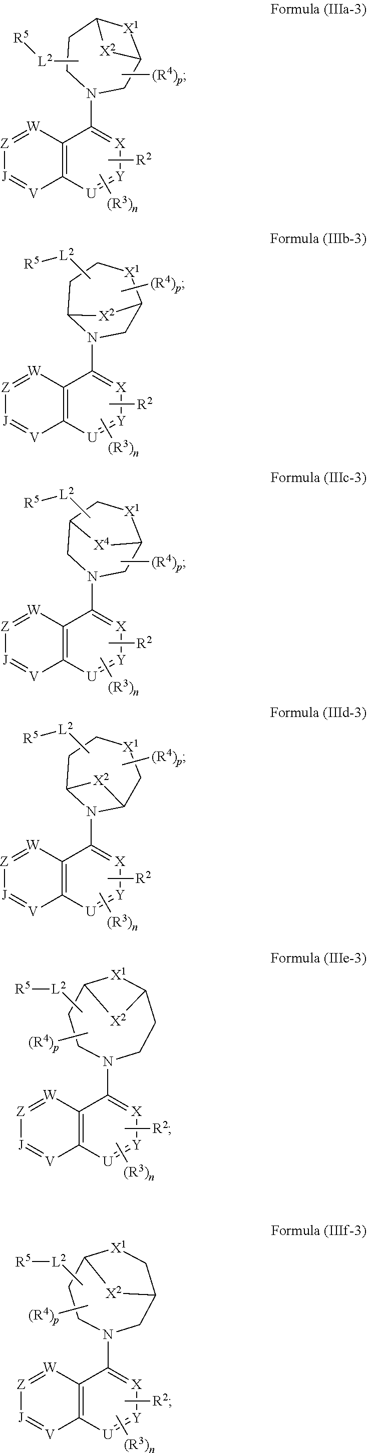

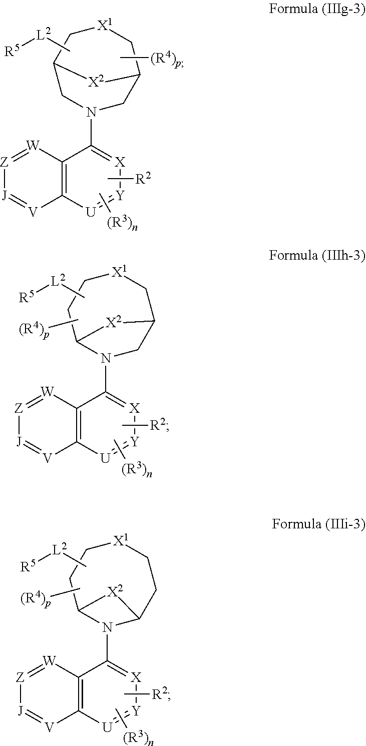

wherein:
X is C or N;
X¹ is selected from C(R⁴)(R⁶), N(R⁴), N(R⁶), O, S, S(O), and S(O)₂;
X² is selected from X³, —CH₂, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)—, —C(H)(R⁴)—, —C(R⁴)₂—, —X³C(R⁴)₂—, —C(R⁴)₂—X³—, —C(H)C(R⁴)₂—, —C(R⁴)₂—C(H)—, and —C(R⁴)₂—C(R⁴)₂—;
X³ is selected from N(R¹), O, S, S(O), and S(O)₂;
X⁴ is selected from X³, —CH₂, —X³CH₂, —CH₂X³—, —CH₂CH₂, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴),X³—, —C(H)C(R⁴)₂—, —C(R⁴), C(H)—, and —C(R⁴), C(R⁴)₂—;
X⁵ is selected from N(R¹), S, S(O), and S(O)₂;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a},
Y is C, S(O), S(O)₂, C(O), or N;
U is C, S(O), S(O)₂, C(O), or N;
Z is Nor C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-4)-(IIIi-4), or a pharmaceutically acceptable salt or solvate thereof:

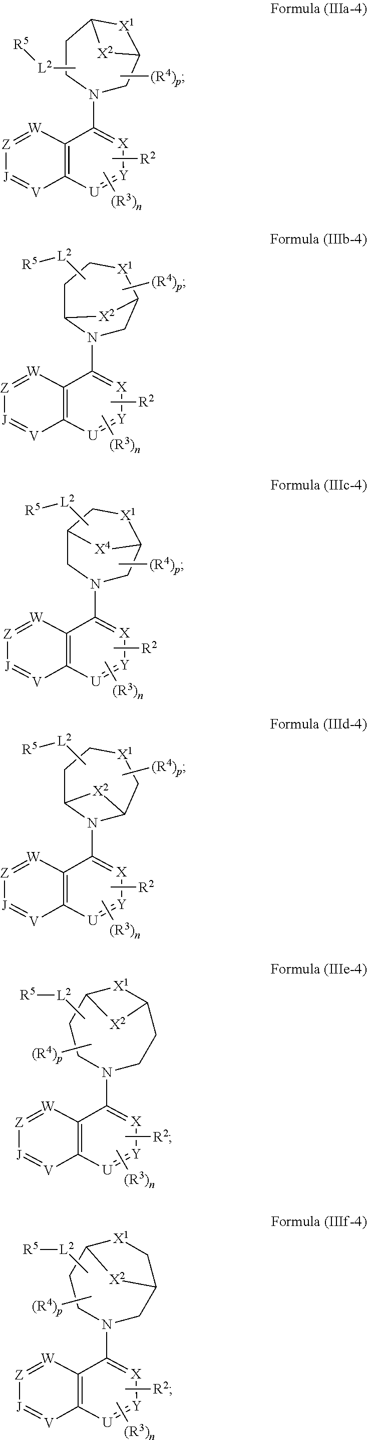

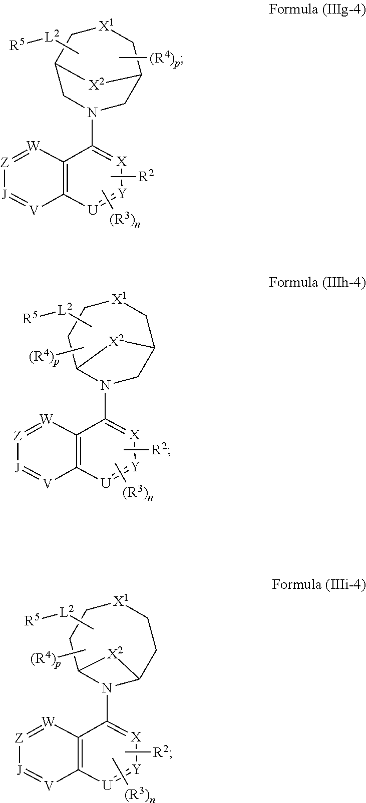

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴), —X³C(R⁴)₂—, —C(R⁴),X³—, —C(H)C(R⁴)₂—, —C(R⁴), C(H)—, and —C(R⁴), C(R⁴)₂—;
X³ is selected from N(R¹), O, S, S(O), and S(O)₂;
X⁴ is selected from X³, —CH₂, —X³CH₂, —CH₂X³—, —CH₂CH₂, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X⁵C(R⁴)₂—, —C(R⁴)₂X³—, —C(H)C(R⁴)₂—, —C(R⁴)₂C(H)—, and —C(R⁴)₂C(R⁴)₂—;
X⁵ is selected from N(R¹), S, S(O), and S(O)₂;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-1)-(IIIb-1) and (IIId-1)-(IIIi-1), or a pharmaceutically acceptable salt or solvate thereof:

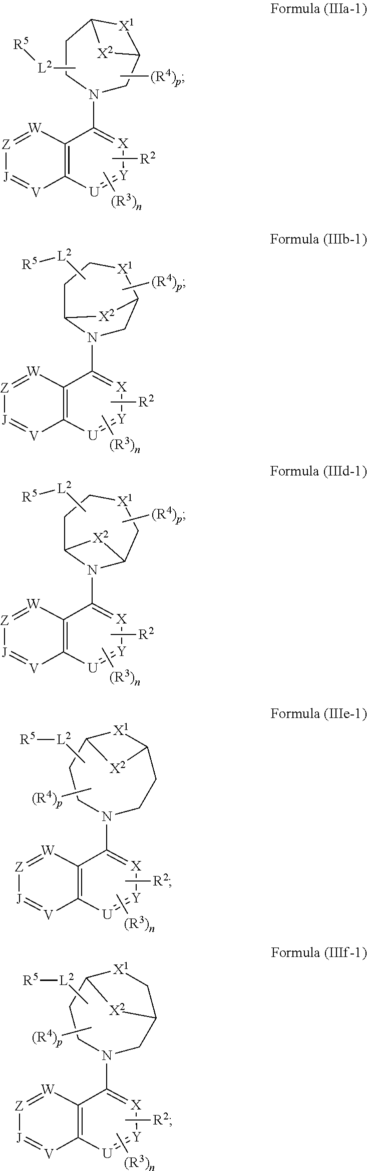

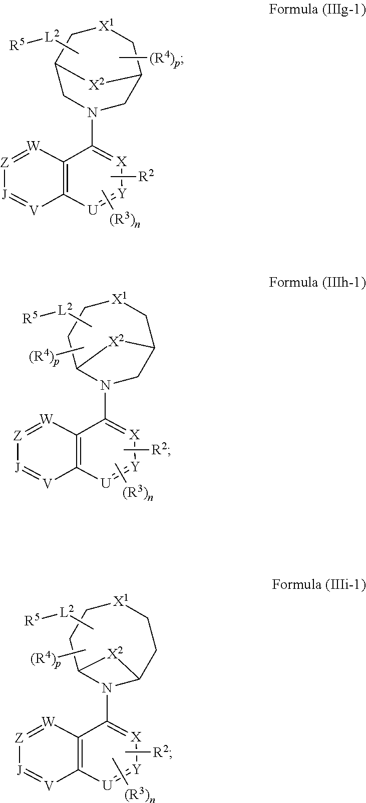

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴), —C(H)(R⁴)₂, —X³C(H)(R⁴)—, —C(H)(R⁴)X³—, —C(H)(R⁴)CH₂—, —C(H)(R⁴)C(H)(R⁴)—, —C(R⁴), —, —X′C(R⁴), —, —C(R⁴),X³—, —C(H)C(R⁴)₂—, —C(R⁴), C(H)—, and —C(R⁴), C(R⁴)₂—;
X³ is selected from N(R¹), O, S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-2)-(IIIb-2) and (IIId-2)-(IIIi-2), or a pharmaceutically acceptable salt or solvate thereof:

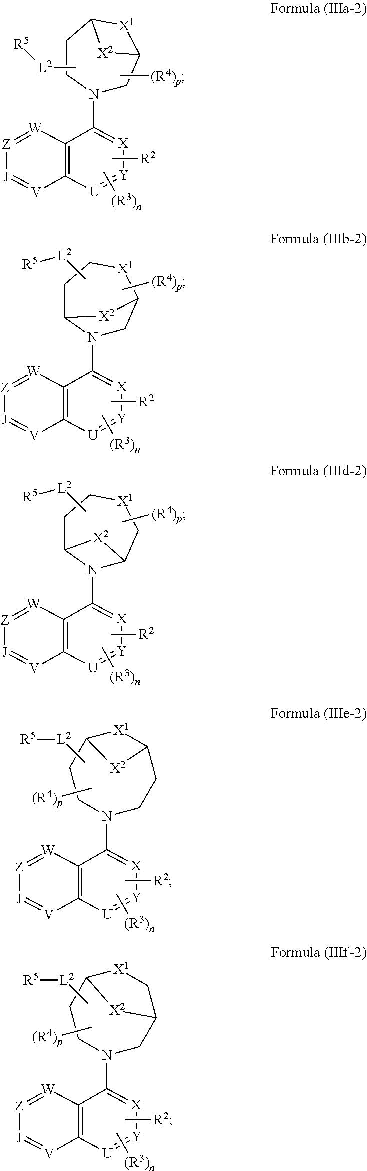

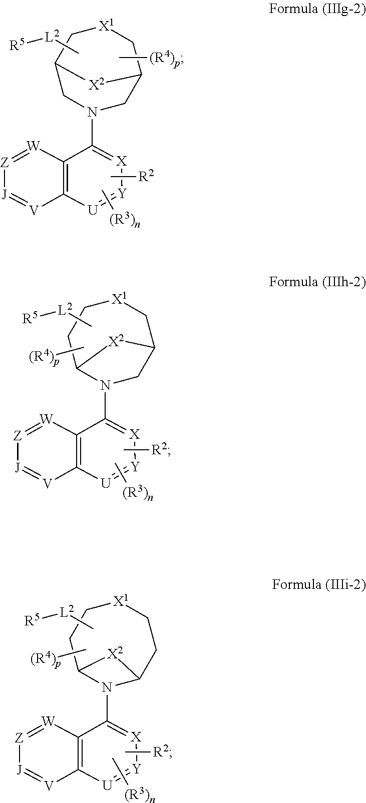

wherein:
X is C or N;
X¹ is selected from C(R⁴)(R⁶), N(R⁴), N(R⁶), O, S, S(O), and S(O);
X² is selected from X′, —CH₂—, —X′CH—, —CH₂X³—, —CH₂CH—, —C(H)(R⁴)—, —((H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X′C(R⁴)₂—, —C(R⁴)₂X³—, —((H)C(R⁴)—, —((R⁴)₂C(H)—, and —C(R⁴)₂C(R⁴)—;
X³ is selected from N(R¹), O, S, S(O), and S(O);
Y is C, S(O), S(O), C(O), or N;
U is C, S(O), S(O), CO), or N;
Z is N or ((R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}, R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-3)-(IIIb-3) and (IIId-3)-(IIIi-3), or a pharmaceutically acceptable salt or solvate thereof:

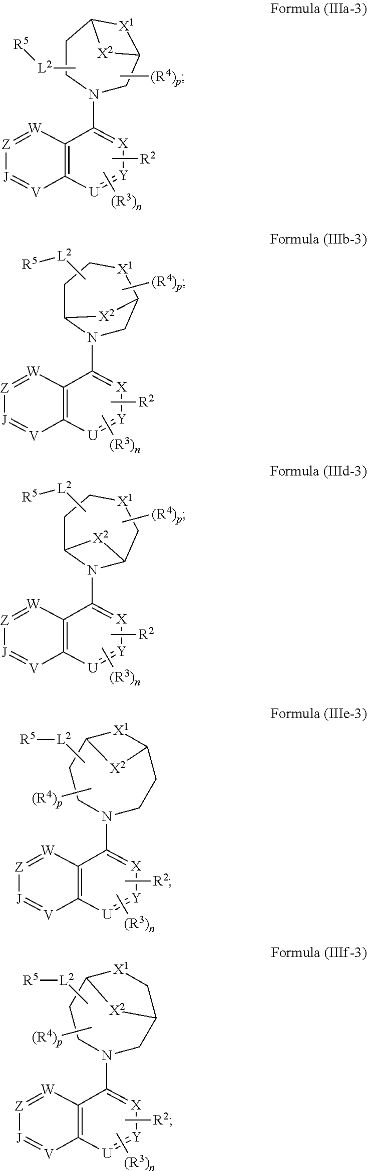

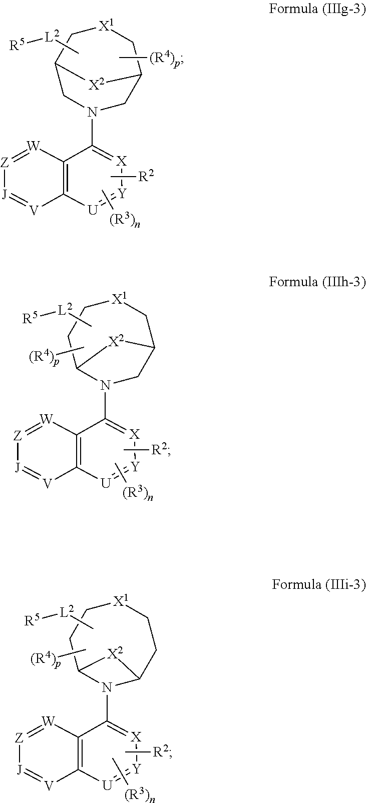

wherein:
X is C or N;
X¹ is selected from)C(R⁴)(R⁶), N(R⁴), N(R⁶), O, S, S(O), and S(O)₂;
X² is selected from X³, —CH₂, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂—X³—, —C(H)C(R⁴)₂—, —C(R⁴)₂—C(H)—, and —C(R⁴)₂C(R⁴)₂—;
X³ is selected from N(R¹), O, S, S(O), and S(O)₂;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂-heterocycloalkyl, —CH₂—C₂₋₉heterocycloalkyl, C₆₋₁₀aryl, —CH₂—C₆₋₁₀aryl, and C₁₋₉heteroaryl, wherein C₁₋₉alkyl, C₂₋₆alkenyl, C₂₋₆alkynyl, C₃₋₆cycloalkyl, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heteroaryl are optionally substituted with one, two, or three R^{20a};
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In another aspect, the disclosure provides a compound of Formula (IIIa-4)-(IIIb-4) and (IIId-4)-(IIIi-4), or a pharmaceutically acceptable salt or solvate thereof:

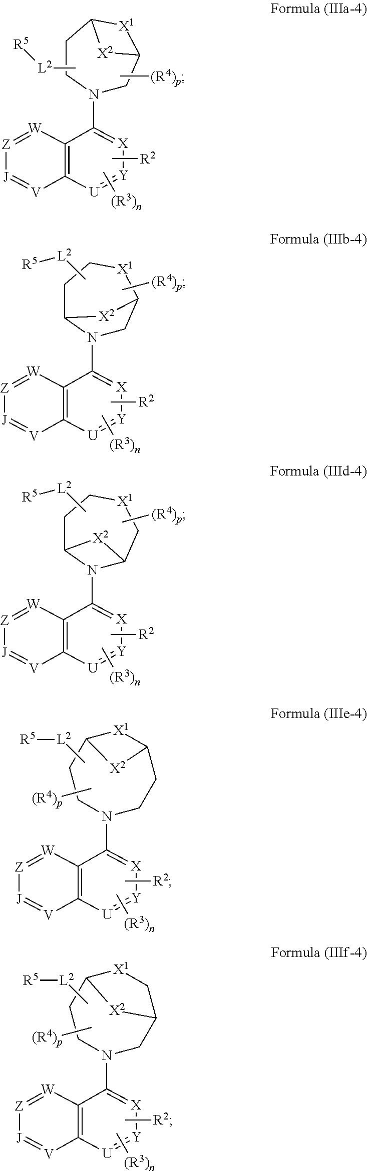

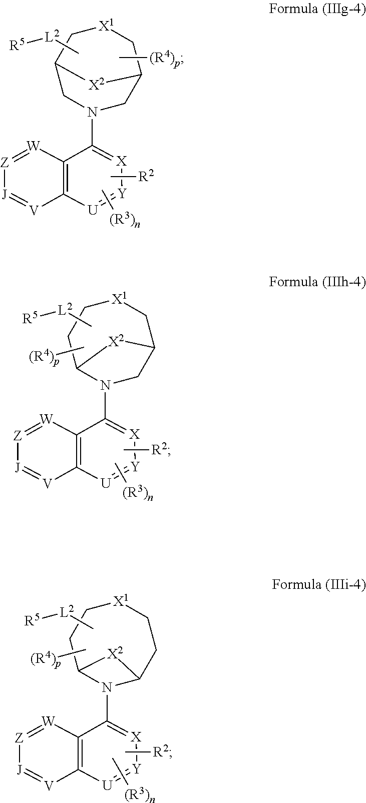

wherein:
X is C or N;
X¹ is selected from)C(R⁴)(R⁶), N(R⁴), N(R⁶), O, S, S(O), and S(O)₂;
X² is selected from X³, —CH₂, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂—X³—, —C(H)C(R⁴)₂—, —C(R⁴)₂—C(H)—, and —C(RA)₂C(R⁴)₂—;
X³ is selected from N(R¹), O, S, S(O), and S(O)₂;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₆heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₉cycloalkyl, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heteroaryl are optionally substituted with one, two, or three R^{20a};
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

In some embodiments is a compound having the structure of Formula (IIIa-1), (IIIa-2), (IIIa-3), or (IIIa-4), or a pharmaceutically acceptable salt or solvate thereof:

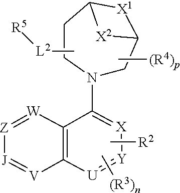

Formula (IIIa-1), (IIIa-2), (IIIa-3), or (IIIa-4) wherein J, U, V, W, X, Y, Z, X¹, X², L², R², R³, n, R⁴, p, and R⁵ are as described herein.

In some embodiments is a compound having the structure of Formula (IIIb-1), (IIIb-2), (IIIb-3), or (IIIb-4), or a pharmaceutically acceptable salt or solvate thereof:

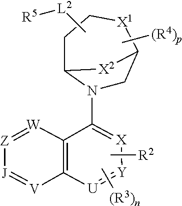

Formula (IIIb-1), (IIIb-2), (IIIb-3), or (IIIb-4) wherein J, U, V, W, X, Y, Z, X¹, X², L², R², R³, n, R⁴, p, and R⁵ are as described herein.

In some embodiments is a compound having the structure of Formula (IIId-1), (IIId-2), (IIId-3), or (IIId-4), or a pharmaceutically acceptable salt or solvate thereof:

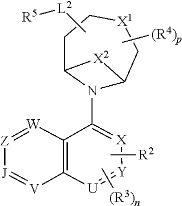

Formula (IIId-1), (IIId-2), (IIId-3), or (IIId-4) wherein J, U, V, W, X, Y, Z, X¹, X², L², R², R³, n, R⁴, p, and R⁵ are as described herein.

In some embodiments is a compound having the structure of Formula (IIIe-1), (IIIe-2), (IIIe-3), or (IIIe-4), or a pharmaceutically acceptable salt or solvate thereof:

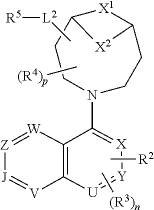

Formula (IIIe-1), (IIIe-2), (IIIe-3), or (IIIe-4) wherein J, U, V, W, X, Y, Z, X¹, X², L², R², R³, n, R⁴, p, and R⁵ are as described herein.

In some embodiments is a compound having the structure of Formula (IIIf-1), (IIIf-2), (IIIf-3), or (IIIf-4), or a pharmaceutically acceptable salt or solvate thereof:

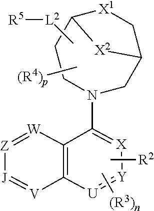

Formula (IIIf-1), (IIIf-2), (IIIf-3), or (IIIf-4) wherein J, U, V, W, X, Y, Z, X¹, X², L², R², R³, n, R⁴, p, and R⁵ are as described herein.

In some embodiments is a compound having the structure of Formula (IIIg-1), (IIIg-2), (IIIg-3), or (IIIg-4), or a pharmaceutically acceptable salt or solvate thereof:

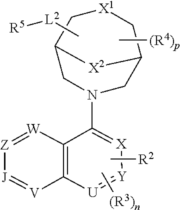

Formula (IIIg-1), (IIIg-2), (IIIg-3), or (IIIg-4) wherein J, U, V, W, X, Y, Z, X¹, X², L², R², R³, n, R⁴, p, and R⁵ are as described herein.

In some embodiments is a compound having the structure of Formula (IIIh-1), (IIIh-2), (IIIh-3) or (IIIh-4), or a pharmaceutically acceptable salt or solvate thereof:

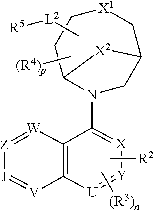

Formula (IIIh-1), (IIIh-2), (IIIh-3) or (IIIh-4) wherein J, U, V, W, X, Y, Z, X¹, X², L², R², R³, n, R⁴, p, and R⁵ are as described herein.

In some embodiments is a compound having the structure of Formula (IIIi-1), (IIIi-2), (IIIi-3) or (IIIi-4), or a pharmaceutically acceptable salt or solvate thereof:

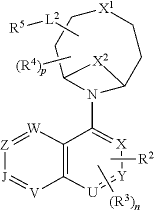

Formula (IIIi-1), (IIIi-2), (IIIi-3) or (IIIi-4) wherein J, U, V, W, X, Y, Z, X¹, X², L², R², R³, n, R⁴, p, and R⁵ are as described herein.

In some embodiments is a compound having the structure of Formula (IIIc-1), (IIIc-2), (IIIc-3), or (IIIc-4), or a pharmaceutically acceptable salt or solvate thereof:

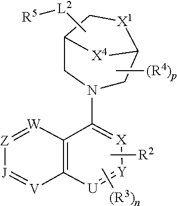

Formula (IIIc-1), (IIIc-2), (IIIc-3), or (IIIc-4) wherein J, U, V, W, X, Y, Z, X¹, X⁴, L², R², R³, n, R⁴, p, and R⁵ are as described herein.

In some embodiments is a compound of Formula (IIIc-1), (IIIc-2), (IIIc-3), or (IIIc-4), or a pharmaceutically acceptable salt or solvate thereof, wherein X⁴ is —N(H)—. In some embodiments is a compound of Formula (IIIc-1), (IIIc-2), (IIIc-3), or (IIIc-4), or a pharmaceutically acceptable salt or solvate thereof, wherein X¹ is —CH₂—. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein R¹ is hydrogen. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (Ille-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein R¹ is -L²-R⁵. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein X⁴ is —NH—.

In some embodiments is a compound of Formula (IIIc-1), (IIIc-2), (IIIc-3), or (IIIc-4), or), or a pharmaceutically acceptable salt or solvate thereof, wherein X⁴ is selected from —CH₂— and —CH₂CH₂—.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein X² is selected from —CH₂— and —CH₂CH₂—.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (Ille-4), (IIIf-4), (IIIg-4), (IIIh-4), or (IIIi-4), or a pharmaceutically acceptable salt or solvate thereof, wherein Y is C. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein Y is N. In some embodiments is a compound of (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein Y is C(O).

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein X is C. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein X is N.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein U is C. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein U is N. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein U is C(O).

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein Z is C(R⁸).

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R⁸ is hydrogen.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein Z is N.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein V is C(R¹⁶). In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein is C(H). In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein V is N.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein J is C(R¹⁷).

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein W is C(R¹⁸). In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein W is C(H). In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein W is N.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IITb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R² is —OR¹².

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹² is C₁₋₆alkyl optionally substituted with one, two, or three R^{20d}. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹² is C₂₋₉heterocycloalkyl optionally substituted with one, two, or three R^{20d}. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁-haloalkyl, C₁₋₆alkoxy, C₁-haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R² is selected from

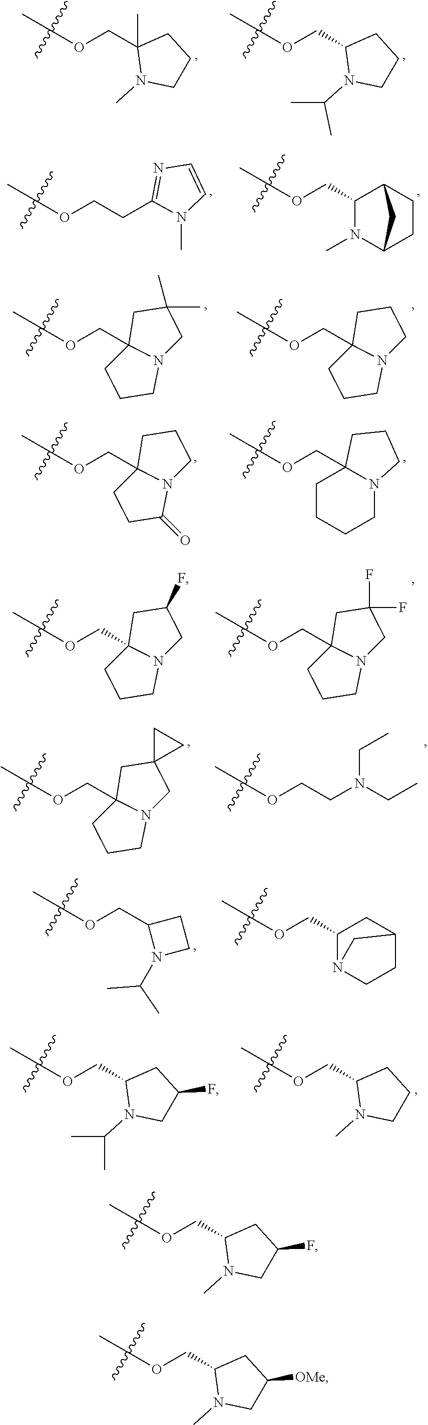

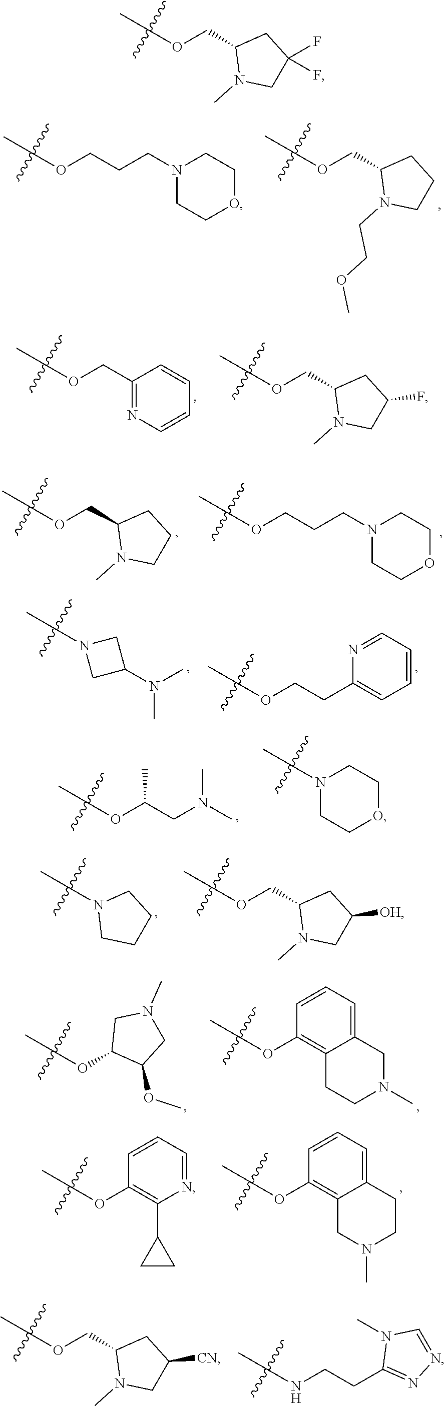

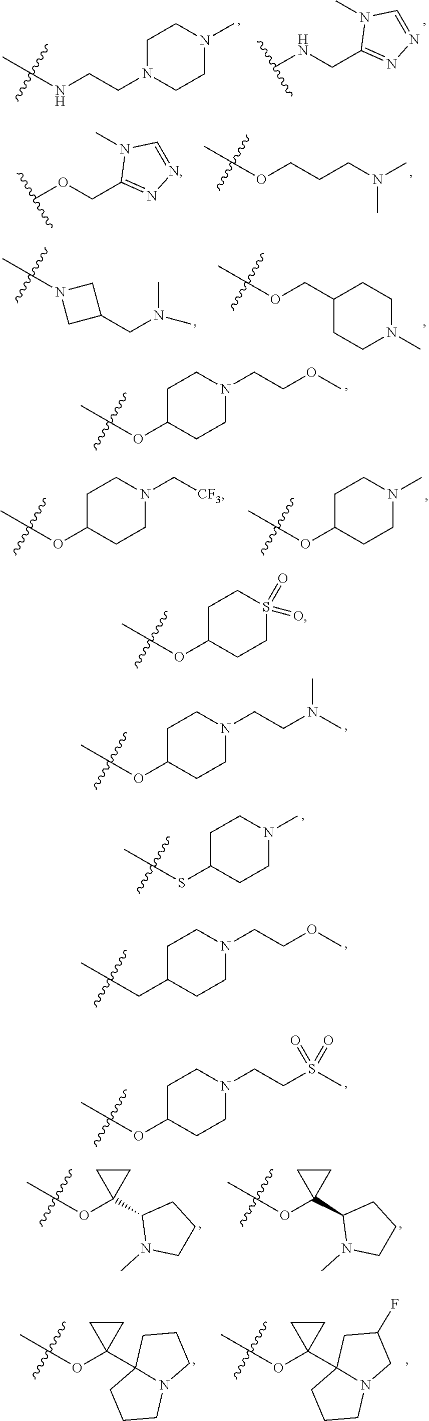

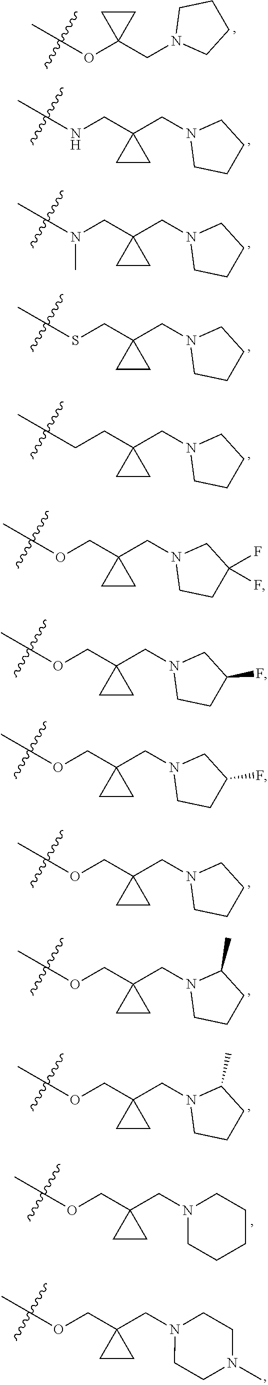

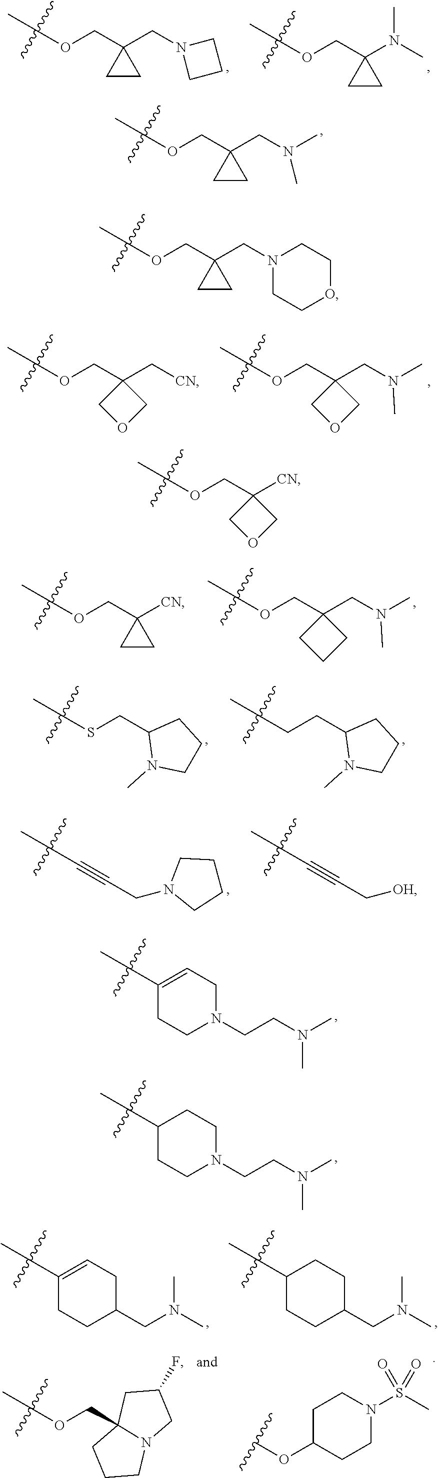

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein L¹ is a bond. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein L¹ is O.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹⁹ is C₁₋₉heteroaryl optionally substituted with one, two, or three R²⁰ⁱ. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R¹⁹ is C₆₋₁₀aryl optionally substituted with one, two, or three R²⁰ⁱ.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein L² is selected from a bond, C₁-C₆alkyl, and —C(O)—. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein L² is a bond. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein L² is, C₁-C₆alkyl.

In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, wherein R⁵ is hydrogen. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, wherein R⁵ is a group other than an electrophilic moiety capable of forming a covalent bond with the cysteine residue at position 12 of a KRAS protein. In some embodiments is a compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, wherein R⁵ is —H, —NH₂, —OH, —NH(C₁₋₆ alkyl),

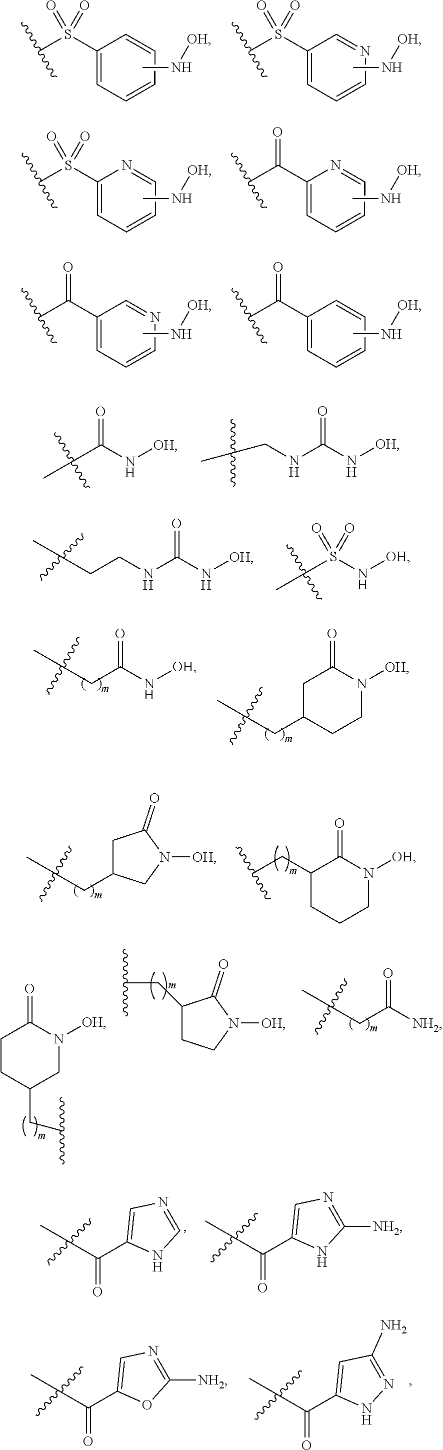

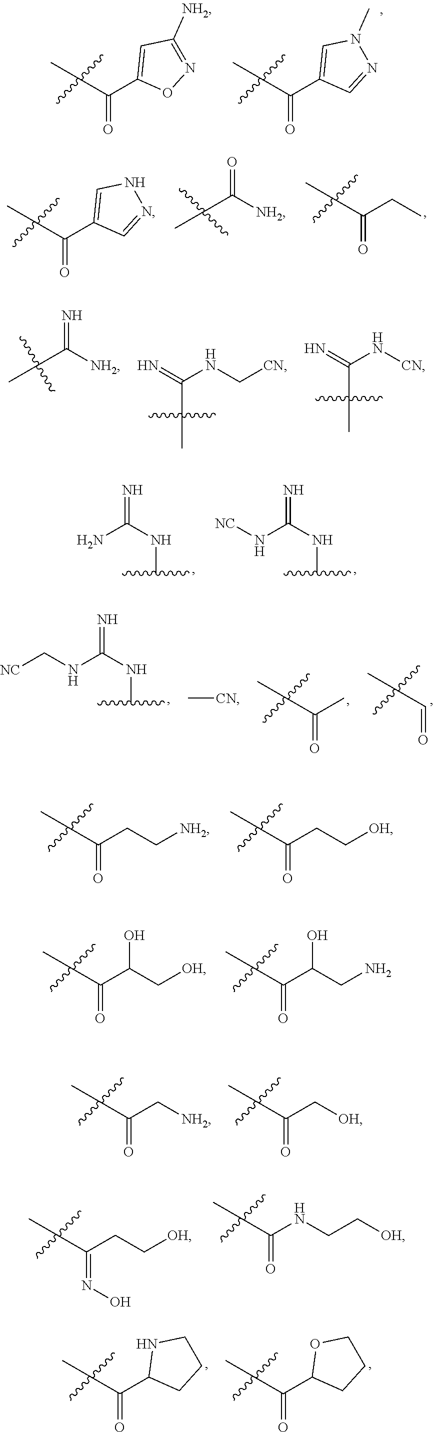

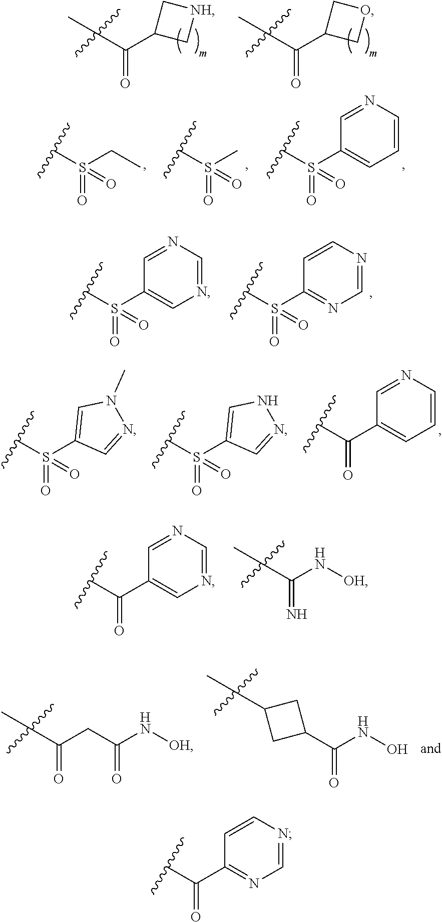

and m, when present, is 0, 1, 2, or 3.

In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C_{2- 9}heterocycloalkyl, C₆₋₁₀aryl, and C₁₋₉heteroaryl are optionally substituted with one, two, or three R^{20a}. In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is halogen. In further embodiments of the subject compound of Formula (IIIa-1), (IIIb-1), (IIIc-1), (IIId-1), (Ille-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —CN. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —OH. In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —NH₂. In further embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —C(O)OH. In select embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —OC(O)NH₂. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —NHC(O)NH₂. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —NHC(O)OH. In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —NHS(O)₂H. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —C(O)NH₂. In some embodiments of the subject compound of Formula (IIIa-1), (IIIb-1), (IIIc-1), (IIId-1), (Ille-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is hydrogen. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is oxo. In further embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is C₁₋₆alkyl optionally substituted with one, two, or three R^{20a}. In select embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is C₃₋₆cycloalkyl optionally substituted with one, two, or three R^{20a}. In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is C₂₋₉heterocycloalkyl optionally substituted with one, two, or three R^{20a}. In some embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is C₆₋₁₀aryl optionally substituted with one, two, or three R^{20a}. In further embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is C₁₋₉heteroaryl optionally substituted with one, two, or three R^{20a}. In select embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —OR¹². In embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is —N(R¹²)(R¹³). In select embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is N(R¹⁴)S(O)₂R¹⁵. In select embodiments of the subject compound of Formula (IIIa-1), (IIIb-1), (IIIc-1), (IIId-1), (IIIe-1), (IIIf-1), (IIIg-1), (IIIh-1), (IIIi-1), (IIIa-3), (IIIb-3), (IIIc-3), (IIId-3), (IIIe-3), (IIIf-3), (IIIg-3), (IIIh-3), or (IIIi-3), or a pharmaceutically acceptable salt or solvate thereof, R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (IIIa-2), (IIIb-2), (IIIc-2), (IIId-2), (IIIe-2), (IIIf-2), (IIIg-2), (IIIh-2), (IIIi-2), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (IIIa-2), (IIIb-2), (IIIc-2), (IIId-2), (IIIe-2), (IIIf-2), (IIIg-2), (IIIh-2), (IIIi-2), (IIIa-4), (IIIb-4), (IIIc-4), (IIId-4), (IIIe-4), (IIIf-4), (IIIg-4), (IIIh-4), or (IIIi-4), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (IIIa), (IIIb), (IIIc), (IIId), (IIIe), (IIIf), (IIIg), (IIIh), or (IIIi), or a pharmaceutically acceptable salt or solvate thereof, wherein R⁵ is selected from the group consisting of

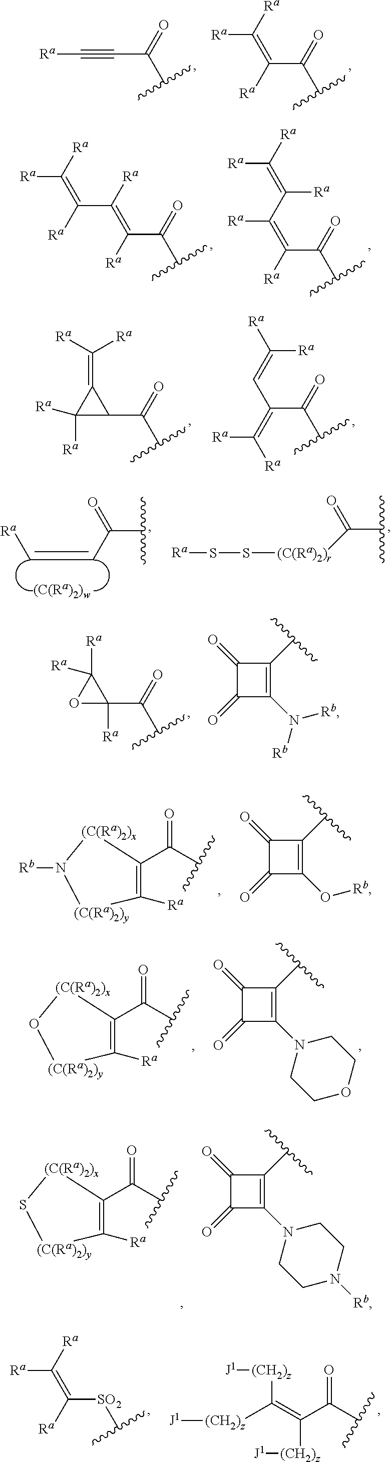

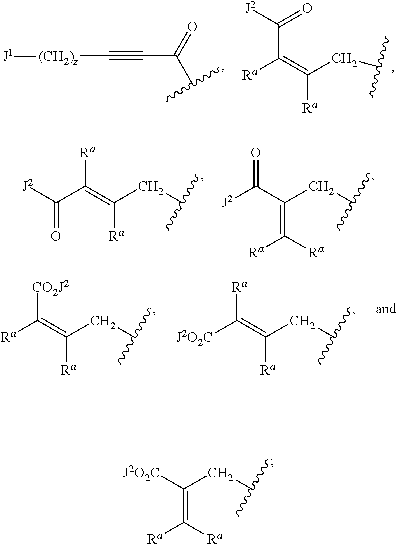

where each R^{a} is independently hydrogen, C₁₋₆alkyl, carboxy, C₁₋₆carboalkoxy, phenyl, C₂₋₇carboalkyl, R^{c}—(C(R^{b})₂)z, R^{c}—(C(R^{b})₂)_{w}-M-(C(R^{b})₂), (R^{a})(R⁶)CH-M-(C(R^{b})₂), or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{e} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

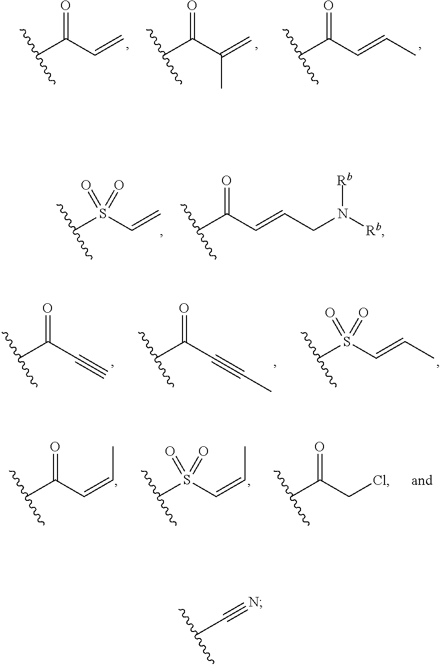

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In another aspect, the disclosure provides a compound of Formula (IVa-1), (IVb-1), or (IVc-1), or a pharmaceutically acceptable salt or solvate thereof:

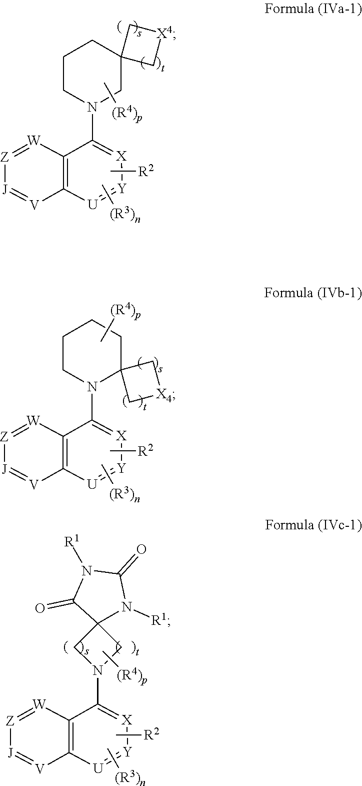

wherein:
X is C or N;
X⁴ is selected from N(R¹), O, S, S(O), S(O), —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is Nor C(R⁴);
V and J are independently selected from N, C(R¹⁶), and C(R¹⁷), wherein one of V and J is C(R¹⁷);
W is N or C(R¹);
L¹ and L² are independently selected from a bond, C₁-C₆alkyl, —O—, —N(R¹⁴)—, —C(O)—, —N(R¹⁴)C(O)—, —C(O)N(R¹⁴), —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

In another aspect, the disclosure provides a compound of Formula (IVa-2), (IVb-2), or (IVc-2), or a pharmaceutically acceptable salt or solvate thereof:

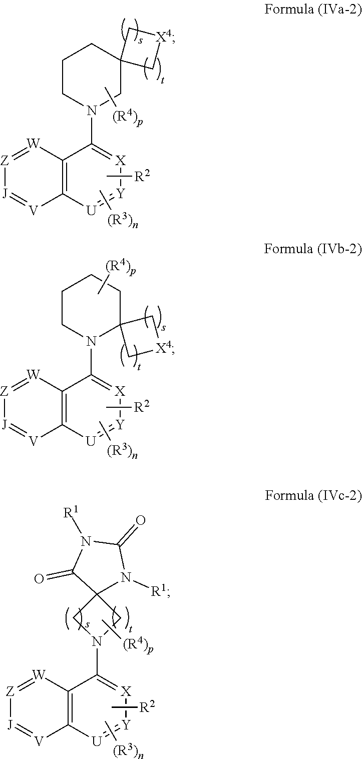

wherein:
X is C or N;
X⁴ is selected from N(R¹), O, S, S(O), S(O)₂, —CH₂—, —C(H)(R⁴)—, —C(R⁴)₂—, and C(H)(-L³-R⁵);
Y is C, S(O), S(O), C(O), or N;
U is C, S(O), S(O)₂, C(O), or N;
Z is Nor C(R⁸);
V and J are independently selected from N, C(R¹⁶), and C(R¹⁷), wherein one of V and J is C(R¹⁷);
W is N or C(R¹⁵);
L¹ and L² are independently selected from a bond, C₁-C₆alkyl, —O—, —N(R¹⁴), —C(O)—, —N(R¹⁴)C(O)—, —C(O)N(R¹⁴)—, —S—, —S(O)₂—, —S(O)—, —S(O)N(R¹⁴)—, —S(O)N(R¹⁴)—, —N(R¹⁴)S(O)—, —N(R¹⁴)S(O)—, —OCON(R¹⁴)—, —N(R¹⁴)C(O)O—, and —N(R¹)C(O)N(R¹)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(—NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

In some embodiments is a compound having the structure of Formula (IVa-1) or (IVa-2), or a pharmaceutically acceptable salt or solvate thereof:

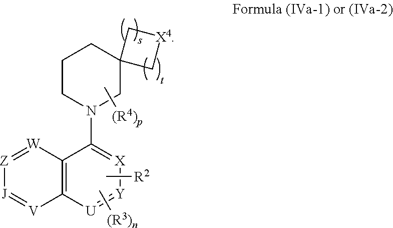

In some embodiments is a compound having the structure of Formula (IVb-1) or (IVb-2), or a pharmaceutically acceptable salt or solvate thereof:

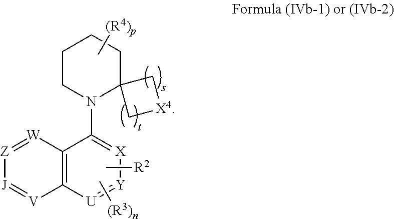

In some embodiments is a compound of Formula (IVa-1), (IVb-1), (IVa-2), or (IVb-2), or a pharmaceutically acceptable salt or solvate thereof, wherein s is 1.

In some embodiments is a compound of Formula (IVa-1), (IVb-1), (IVa-2), or (IVb-2), or a pharmaceutically acceptable salt or solvate thereof, wherein t is 1 or 2.

In some embodiments is a compound of Formula (IVa-1), (IVb-1), (IVa-2), or (IVb-2), or a pharmaceutically acceptable salt or solvate thereof, wherein X⁴ is N(R¹).

In some embodiments is a compound having the structure of Formula (IVc-1) or (IVc-2), or a pharmaceutically acceptable salt or solvate thereof:

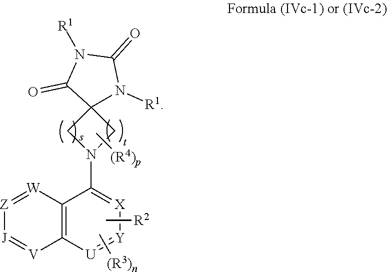

In some embodiments is a compound of Formula (IVa-1), (IVb-1), (IVc-1), (IVa-2), (IVb-2), or (IVc-2), or a pharmaceutically acceptable salt or solvate thereof, wherein R¹ is hydrogen. In some embodiments is a compound of Formula (IVa-1), IVb-1), (IVc-1), (IVa-2), (IVb-2), or (IVc-2), or (IVc), or a pharmaceutically acceptable salt or solvate thereof, wherein R¹ is -L²-R⁵.

In some embodiments is a compound of Formula (IVa-1), (IVb-1), (IVc-1), (IVa-2), (IVb-2), or (IVc-2), or a pharmaceutically acceptable salt or solvate thereof, wherein Y is C. In some embodiments is a compound of Formula (IVa-1), (IVb-1), (IVc-1), (IVa-2), (IVb-2), or (IVc-2), or a pharmaceutically acceptable salt or solvate thereof, wherein Y is N. In some embodiments is a compound of Formula (IVa-1), (IVb-1), (IVc-1), (IVa-2), (IVb-2), or (IVc-2), or a pharmaceutically acceptable salt or solvate thereof, wherein Y is C(O).

In some embodiments is a compound of Formula (IVa-1), (IVb-1), (IVc-1), (IVa-2), (IVb-2), or (IVc-2), or a pharmaceutically acceptable salt or solvate thereof, wherein X is C. In some embodiments is a compound of Formula (IVa-1), (IVb-1), (IVc-1), (IVa-2), (IVb-2), or (IVc-2), or a pharmaceutically acceptable salt or solvate thereof, wherein X is N.

In some embodiments is a compound of Formula (IVa-1), (IVb-1), (IVc-1), (IVa-2), (IVb-2), or (IVc-2), or a pharmaceutically acceptable salt or solvate thereof, wherein U is C. In some embodiments is a compound of Formula (IVa-1), IVb-1), (IVc-1), (IVa-2), (IVb-2), or (IVc-2), or a pharmaceutically acceptable salt or solvate thereof, wherein U is N. In some embodiments is a compound of Formula (IVa-1), (IVb-1), (IVc-1), (IVa-2), (IVb-2), or (IVc-2), or a pharmaceutically acceptable salt or solvate thereof, wherein U is C(O).

In some embodiments is a compound of Formula (IVa-1), (IVb-1), (IVc-1), (IVa-2), (IVb-2), or (IVc-2), or a pharmaceutically acceptable salt or solvate thereof, wherein Z is C(R⁸).

In some embodiments is a compound of Formula (IVa-1), (IVb-1), (IVc-1), (IVa-2), (IVb-2), or (IVc-2), or a pharmaceutically acceptable salt or solvate thereof, wherein R⁸ is hydrogen.

In some embodiments is a compound of Formula (IVa-1), (IVb-1), (IVc-1), (IVa-2), (IVb-2), or (IVc-2), or a pharmaceutically acceptable salt or solvate thereof, wherein Z is N.

In some embodiments is a compound of Formula (IVa-1), (IVb-1), (IVc-1), (IVa-2), (IVb-2), or (IVc-2), or a pharmaceutically acceptable salt or solvate thereof, wherein V is C(R¹⁶). In some embodiments is a compound of Formula (IVa-1), (IVb-1), (IVc-1), (IVa-2), (IVb-2), or (IVc-2), or a pharmaceutically acceptable salt or solvate thereof, wherein is C(H). In some embodiments is a compound of Formula (IVa-1), (IVb-1), (IVc-1), (IVa-2), (IVb-2), or (IVc-2), or a pharmaceutically acceptable salt or solvate thereof, wherein V is N.

In some embodiments is a compound of Formula (IVa-1), (IVb-1), (IVc-1), (IVa-2), (IVb-2), or (IVc-2), or a pharmaceutically acceptable salt or solvate thereof, wherein J is C(R¹⁷).

In some embodiments is a compound of Formula (IVa-1), (IVb-1), (IVc-1), (IVa-2), (IVb-2), or (IVc-2), or a pharmaceutically acceptable salt or solvate thereof, wherein W is C(R¹⁸). In some embodiments is a compound of Formula (IVa-1), IVb-1), (IVc-1), (IVa-2), (IVb-2), or (IVc-2), or a pharmaceutically acceptable salt or solvate thereof, wherein W is C(H). In some embodiments is a compound of Formula (IVa-1), (IVb-1), (IVc-1), (IVa-2), (IVb-2), or (IVc-2), or a pharmaceutically acceptable salt or solvate thereof, wherein W is N.

In some embodiments is a compound of Formula (IVa-1), (IVb-1), (IVc-1), (IVa-2), (IVb-2), or (IVc-2), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (IVa-1), (IVb-1), (IVc-1), (IVa-2), (IVb-2), or (IVc-2), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (IVa-1), (IVb-1), (IVc-1), (IVa-2), (IVb-2), or (IVc-2), or a pharmaceutically acceptable salt or solvate thereof, wherein R² is —OR¹².

In some embodiments is a compound of Formula (IVa-1), (IVb-1), (IVc-1), (IVa-2), (IVb-2), or (IVc-2), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (IVa-1), (IVb-1), (IVc-1), (IVa-2), (IVb-2), or (IVc-2), or a pharmaceutically acceptable salt or solvate thereof, wherein R¹² is C₁₋₆alkyl optionally substituted with one, two, or three R^{20d}. In some embodiments is a compound of Formula (IVa-1), (IVb-1), (IVc-1), (IVa-2), (IVb-2), or (IVc-2), or a pharmaceutically acceptable salt or solvate thereof, wherein R¹² is C₂₋₉heterocycloalkyl optionally substituted with one, two, or three R^{20d}. In some embodiments is a compound of Formula (IVa-1), (IVb-1), (IVc-1), (IVa-2), (IVb-2), or (IVc-2), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (IVa-1), (IVb-1), (IVc-1), (IVa-2), (IVb-2), or (IVc-2), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (IVa-1), (IVb-1), (IVc-1), (IVa-2), (IVb-2), or (IVc-2), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (IVa-1), (IVb-1), (IVc-1), (IVa-2), (IVb-2), or (IVc-2), or a pharmaceutically acceptable salt or solvate thereof, wherein R² is selected from

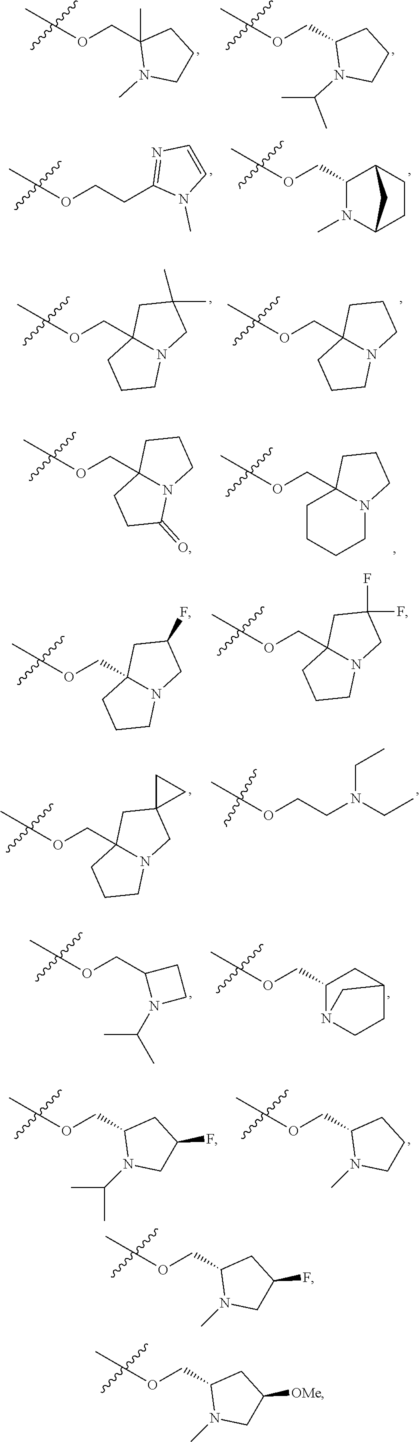

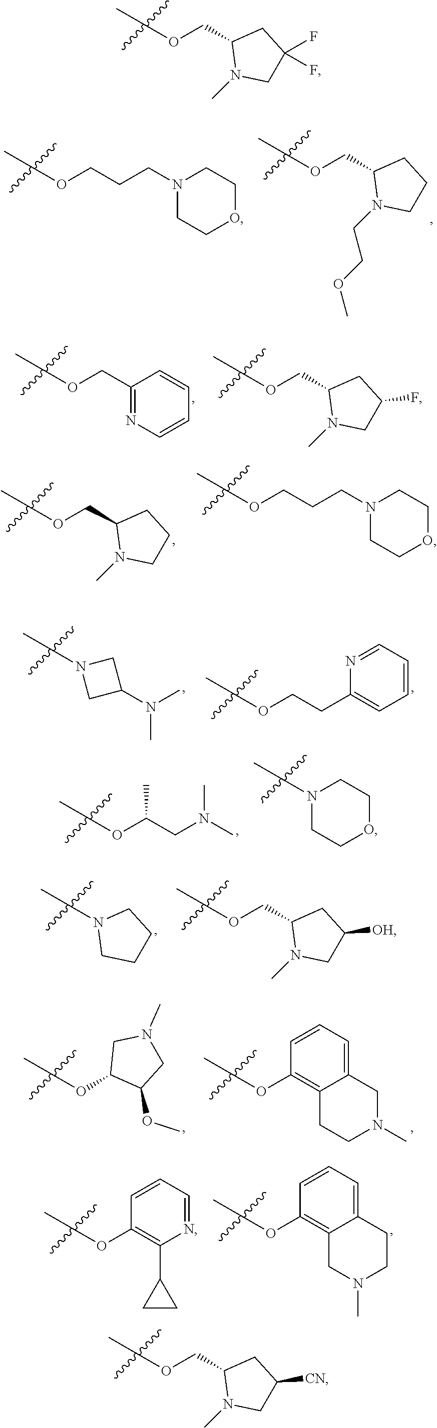

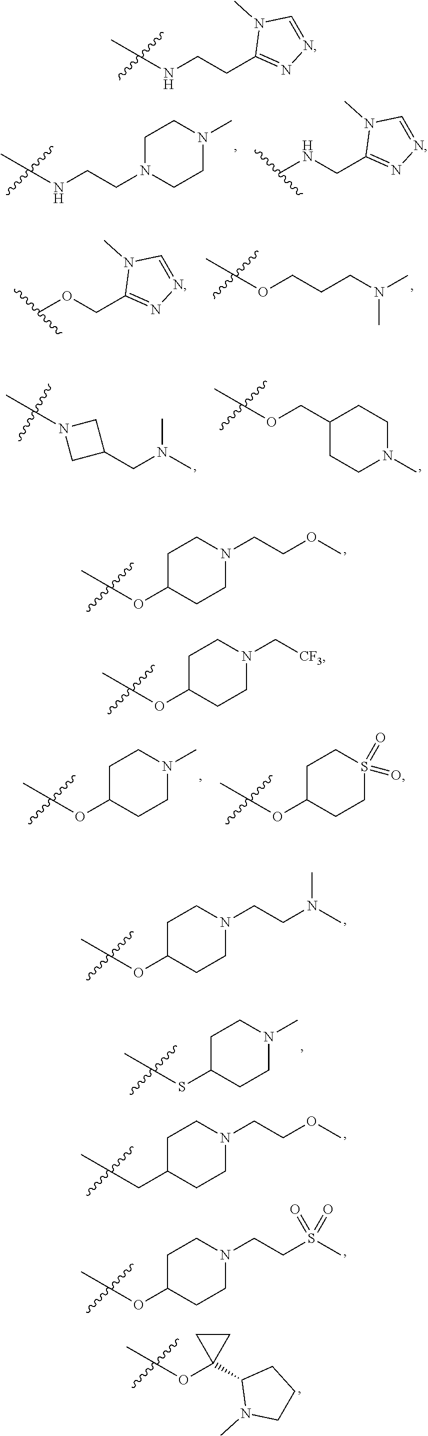

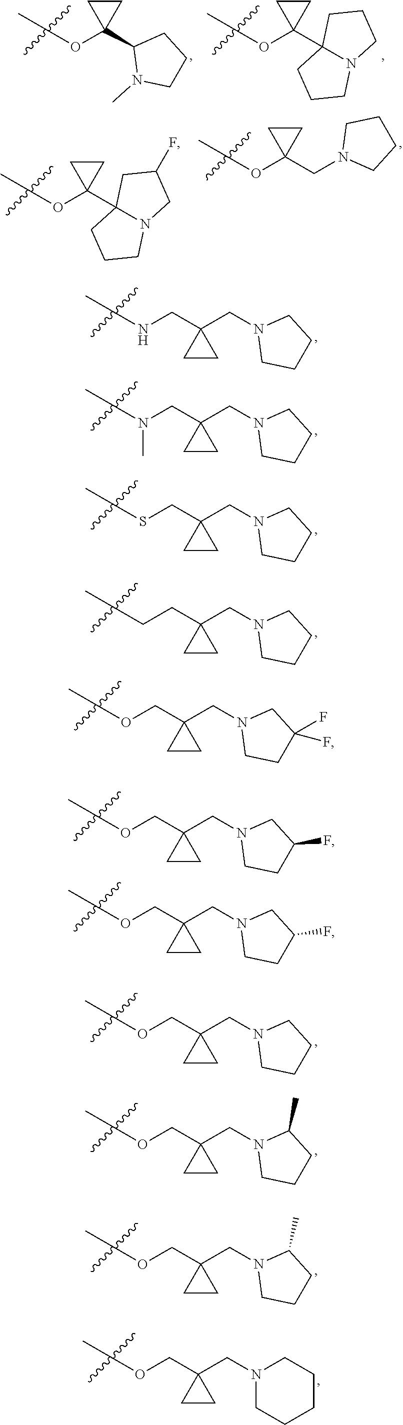

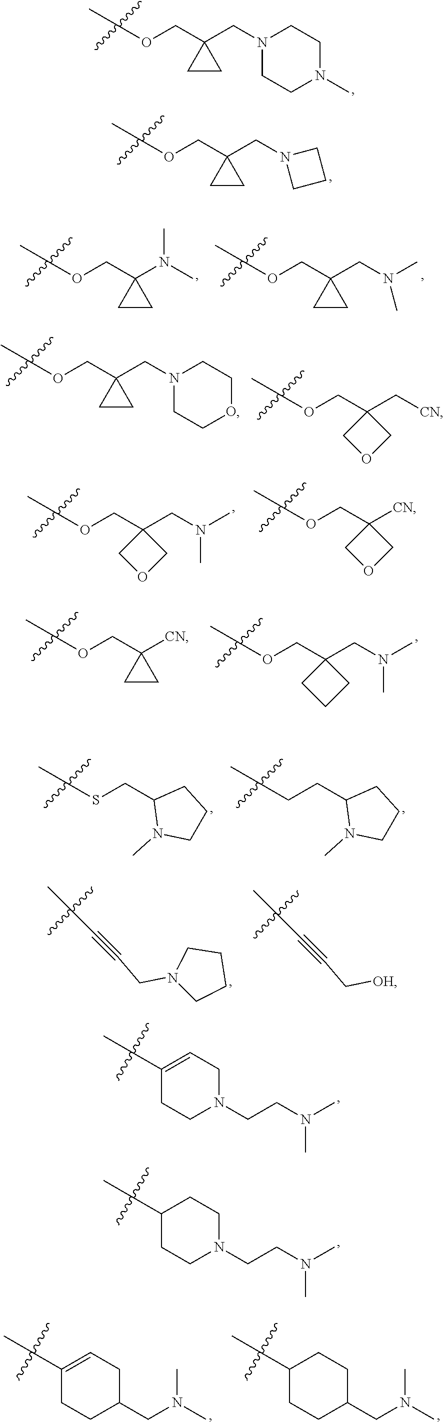

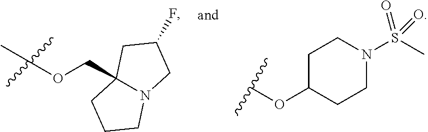 [SMILES: *OC[C@]12CCCN1C[C@@H](F)C2 ; *OC1CCN(S(C)(=O)=O)CC1]

In some embodiments is a compound of Formula (IVa-1), (IVb-1), (IVc-1), (IVa-2), (IVb-2), or (IVc-2), or a pharmaceutically acceptable salt or solvate thereof, wherein L¹ is a bond. In some embodiments is a compound of Formula (IVa-1), IVb-1), (IVc-1), (IVa-2), (IVb-2), or (IVc-2), or a pharmaceutically acceptable salt or solvate thereof, wherein L¹ is O.

In some embodiments is a compound of Formula (IVa-1), IVb-1), (IVc-1), (IVa-2), (IVb-2), or (IVc-2), or a pharmaceutically acceptable salt or solvate thereof, wherein R¹⁹ is C₁₋₉heteroaryl optionally substituted with one, two, or three R²⁰ⁱ. In some embodiments is a compound of Formula (IVa-1), (IVb-1), (IVc-1), (IVa-2), (IVb-2), or (IVc-2), or a pharmaceutically acceptable salt or solvate thereof, wherein R¹⁹ is C₆₋₁₀aryl optionally substituted with one, two, or three R²⁰ⁱ.

In some embodiments is a compound of Formula (IVa-1), (IVb-1), (IVc-1), (IVa-2), (IVb-2), or (IVc-2), or a pharmaceutically acceptable salt or solvate thereof, wherein L² is selected from a bond, C₁-C₆alkyl, and —C(O)—. In some embodiments is a compound of Formula (IVa-1), (IVb-1), (IVc-1), (IVa-2), (IVb-2), or (IVc-2), or a pharmaceutically acceptable salt or solvate thereof, wherein L² is a bond. In some embodiments is a compound of Formula (IVa-1), IVb-1), (IVc-1), (IVa-2), (IVb-2), or (IVc-2), or a pharmaceutically acceptable salt or solvate thereof, wherein L² is, C₁-C₆alkyl.

In some embodiments is a compound of Formula (IVa-1), (IVb-1), or (IVc-1), or a pharmaceutically acceptable salt or solvate thereof, wherein each R⁵ is independently hydrogen. In some embodiments is a compound of Formula (IVa-1), (IVb-1), or (IVc-1), or a pharmaceutically acceptable salt or solvate thereof, wherein each R⁵ is independently a group other than an electrophilic moiety capable of forming a covalent bond with the cysteine residue at position 12 of a KRAS protein. In some embodiments is a compound of Formula (IVa-1), (IVb-1), or (IVc-1) or a pharmaceutically acceptable salt or solvate thereof, wherein each R⁵ is independently —H, —NH₂, —OH, —NH(C₁₋₆ alkyl)

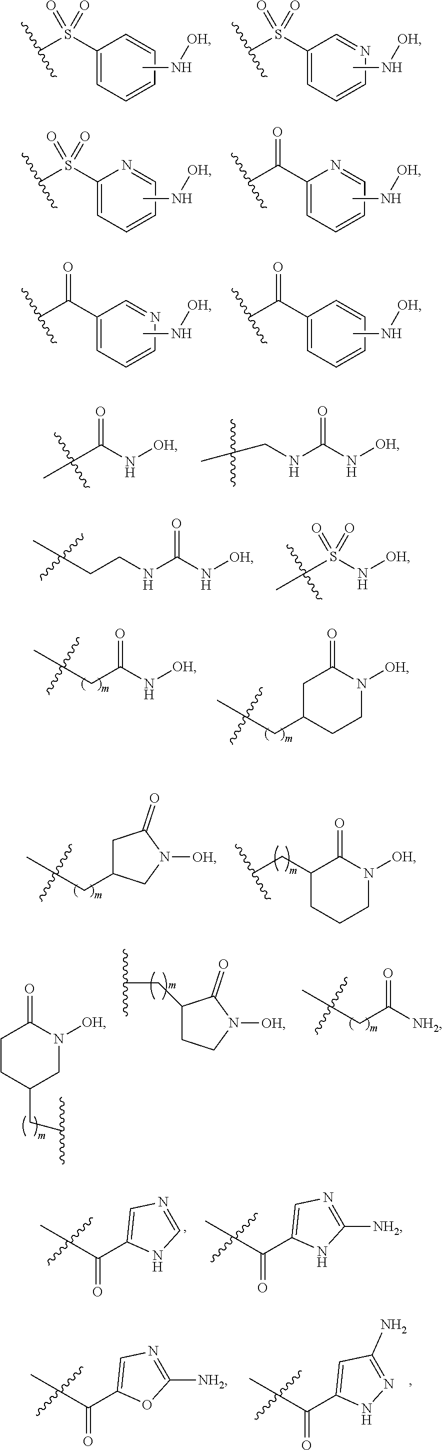

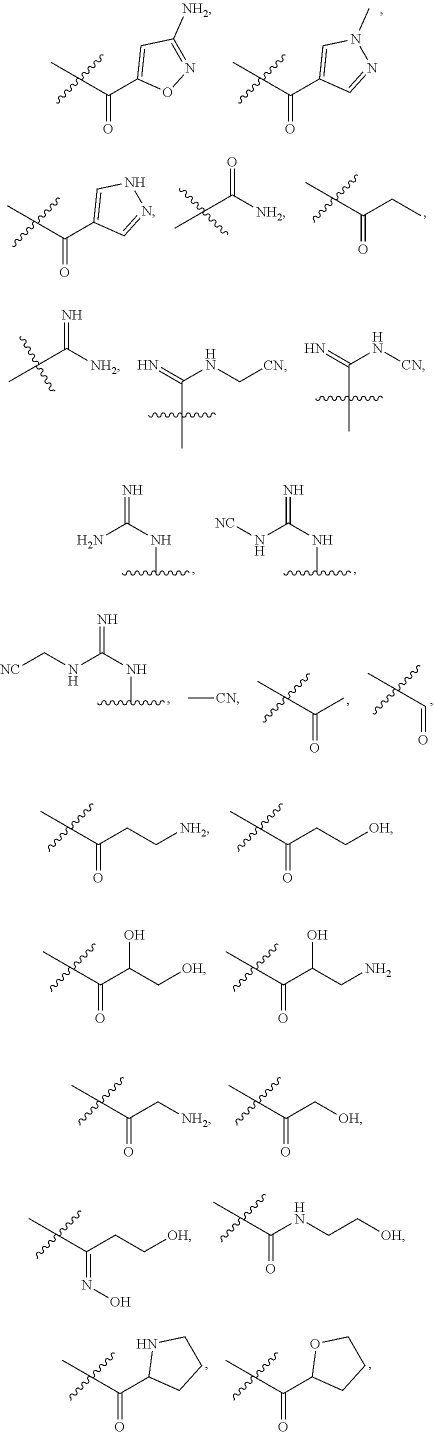

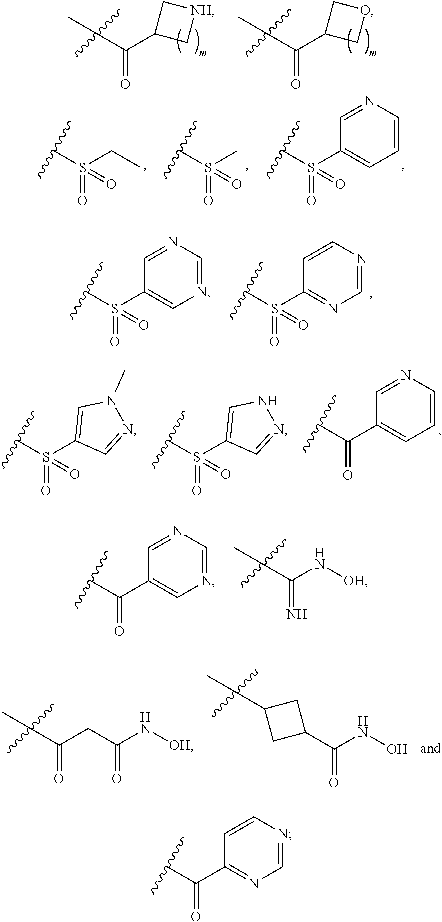

and m, when present, is 0, 1, 2, or 3.

In some embodiments of the subject compound of Formula (IVa-1), (IVb-1), or (IVc-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C_{2- 9}heterocycloalkyl, C₆₋₁₀aryl, and C₁₋₉heteroaryl are optionally substituted with one, two, or three R^{20a}. In some embodiments of the subject compound of Formula (IVa-1), (IVb-1), or (IVc-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (IVa-1), (IVb-1), or (IVc-1), or a pharmaceutically acceptable salt or solvate thereof, each R⁵ is independently halogen. In further embodiments of the subject compound of Formula (IVa-1), (IVb-1), or (IVc-1), or a pharmaceutically acceptable salt or solvate thereof, each R⁵ is independently —CN. In embodiments of the subject compound of Formula (IVa-1), (IVb-1), or (IVc-1), or a pharmaceutically acceptable salt or solvate thereof, each R⁵ is independently —OH. In some embodiments of the subject compound of Formula (IVa-1), (IVb-1), or (IVc-1), or a pharmaceutically acceptable salt or solvate thereof, each R⁵ is independently —NH₂. In further embodiments of the subject compound of Formula (IVa-1), (IVb-1), or (IVc-1), or a pharmaceutically acceptable salt or solvate thereof, each R⁵ is independently —C(O)OH. In select embodiments of the subject compound of Formula (IVa-1), (IVb-1), or (IVc-1), or a pharmaceutically acceptable salt or solvate thereof, each R⁵ is independently —OC(O)NH₂. In embodiments of the subject compound of Formula (IVa-1), (IVb-1), or (IVc-1), or a pharmaceutically acceptable salt or solvate thereof, each R⁵ is independently —NHC(O)NH₂. In embodiments of the subject compound of Formula (IVa-1), (IVb-1), or (IVc-1), or a pharmaceutically acceptable salt or solvate thereof, each R⁵ is independently —NHC(O)OH. In some embodiments of the subject compound of Formula (IVa-1), (IVb-1), or (IVc-1), or a pharmaceutically acceptable salt or solvate thereof, each R⁵ is independently —NHS(O)₂H. In embodiments of the subject compound of Formula (IVa-1), (IVb-1), or (IVc-1), or a pharmaceutically acceptable salt or solvate thereof, each R⁵ is independently —C(O)NH₂. In some embodiments of the subject compound of Formula (IVa-1), (IVb-1), or (IVc-1), or a pharmaceutically acceptable salt or solvate thereof, each R⁵ is independently each R⁵ is independently hydrogen. In embodiments of the subject compound of Formula (IVa-1), (IVb-1), or (IVc-1), or a pharmaceutically acceptable salt or solvate thereof, each R⁵ is independently oxo. In further embodiments of the subject compound of Formula (IVa-1), (IVb-1), or (IVc-1), or a pharmaceutically acceptable salt or solvate thereof, each R⁵ is independently C₁₋₆alkyl optionally substituted with one, two, or three R^{20a}. In select embodiments of the subject compound of Formula (IVa-1), (IVb-1), or (IVc-1), or a pharmaceutically acceptable salt or solvate thereof, each R⁵ is independently C₃₋₆cycloalkyl optionally substituted with one, two, or three R^{20a}. In embodiments of the subject compound of Formula (IVa-1), (IVb-1), or (IVc-1), or a pharmaceutically acceptable salt or solvate thereof, each R⁵ is independently C₂₋₉heterocycloalkyl optionally substituted with one, two, or three R^{20a}. In some embodiments of the subject compound of Formula (IVa-1), (IVb-1), or (IVc-1), or a pharmaceutically acceptable salt or solvate thereof, each R⁵ is independently C₆₋₁₀aryl optionally substituted with one, two, or three R^{20a}. In further embodiments of the subject compound of Formula (IVa-1), (IVb-1), or (IVc-1), or a pharmaceutically acceptable salt or solvate thereof, each R⁵ is independently each R⁵ is independently C₁₋₉heteroaryl optionally substituted with one, two, or three R^{20a}. In select embodiments of the subject compound of Formula (IVa-1), (IVb-1), or (IVc-1), or a pharmaceutically acceptable salt or solvate thereof, each R⁵ is independently —OR¹². In embodiments of the subject compound of Formula (IVa-1), (IVb-1), or (IVc-1), or a pharmaceutically acceptable salt or solvate thereof, each R⁵ is independently —N(R¹²)(R¹³). In select embodiments of the subject compound of Formula (IVa-1), (IVb-1), or (IVc-1), or a pharmaceutically acceptable salt or solvate thereof, each R⁵ is independently N(R¹⁴)S(O)₂R¹⁵. In select embodiments of the subject compound of Formula (IVa-1), (IVb-1), or (IVc-1), or a pharmaceutically acceptable salt or solvate thereof, each R⁵ is independently —S(O)₂R¹⁵. In embodiments;
each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (IVa-2), (IVb-2), or (IVc-2), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (IVa-2), (IVb-2), or (IVc-2), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (IVa-2), (IVb-2), or (IVc-2), or a pharmaceutically acceptable salt or solvate thereof, wherein R⁵ is selected from the group consisting of

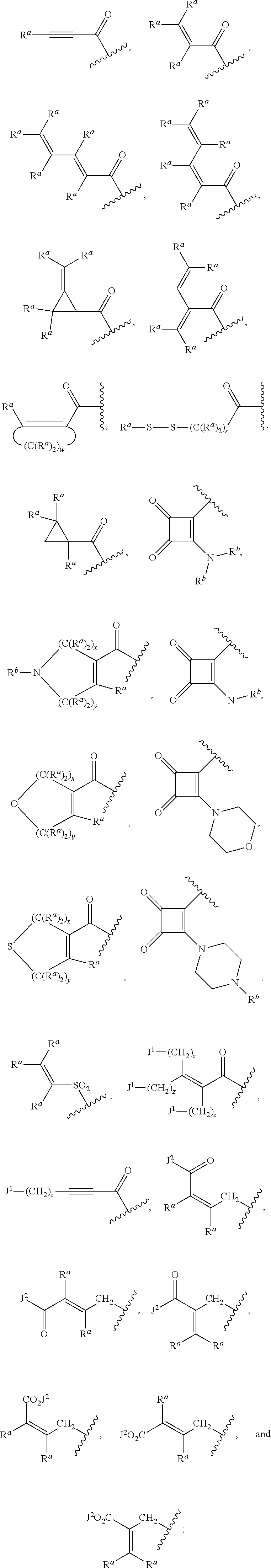

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-(C(R^{b})₂)ᵣ—, (R^{d})(R⁶)CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)—NR^{b}R^{b}, or —(C(R^{b})₂), —OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

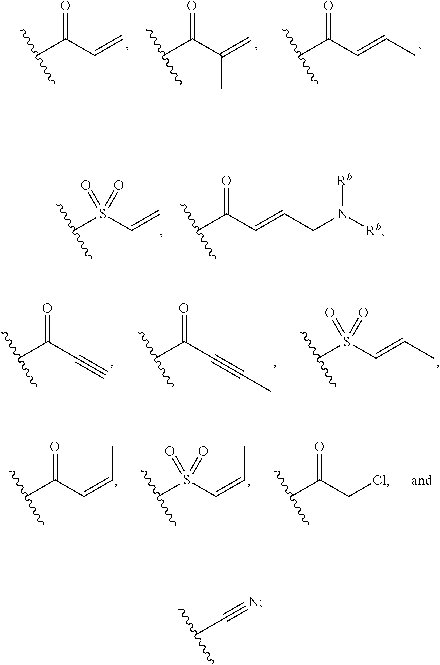

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In another aspect, the disclosure provides a compound of Formula (Va-1), (Vb-1), or (Vc-1), or a pharmaceutically acceptable salt or solvate thereof:

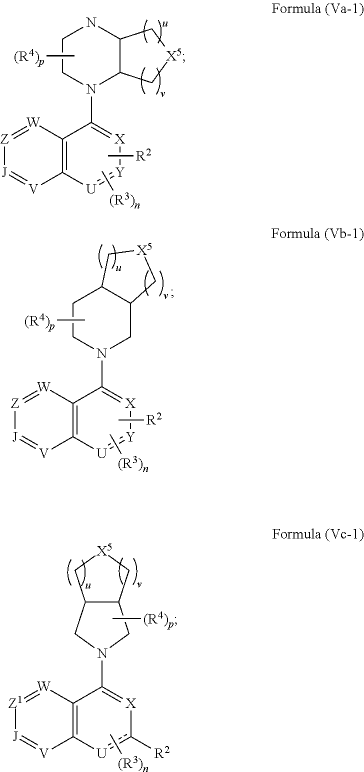

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

In another aspect, the disclosure provides a compound of Formula (Va-2), (Vb-2), or (Vc-2), or a pharmaceutically acceptable salt or solvate thereof:

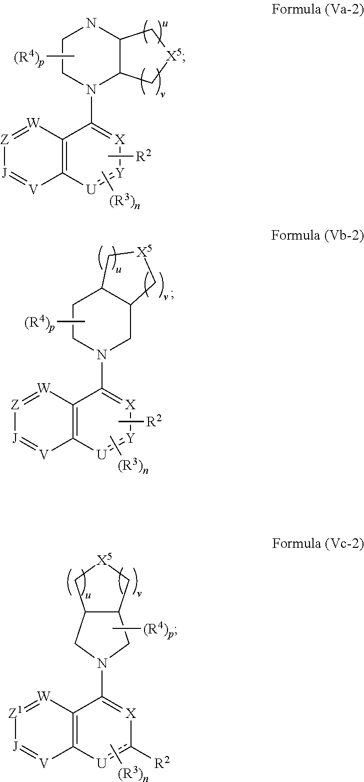

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁵ is independently an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

In some embodiments is a compound having the structure of Formula (Va-1) or (Va-2), or a pharmaceutically acceptable salt or solvate thereof:

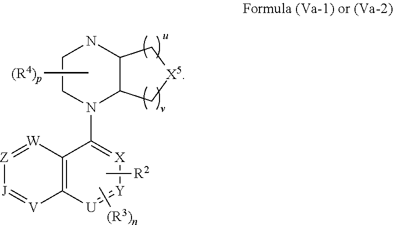

In some embodiments is a compound having the structure of Formula (Vb-1) or (Vb-2), or a pharmaceutically acceptable salt or solvate thereof:

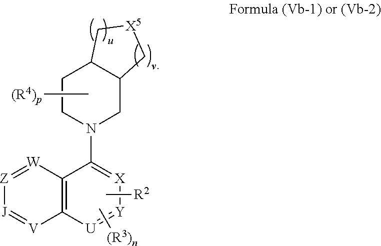

In some embodiments is a compound having the structure of Formula (Vc-1) or (Vc-2), or a pharmaceutically acceptable salt or solvate thereof:

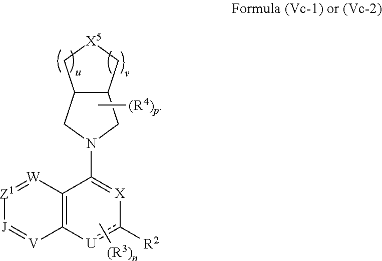

In some embodiments is a compound of Formula (Va-1), (Vb-1), (Vc-1), (Va-2), (Vb-2), or (Vc-2), or a pharmaceutically acceptable salt or solvate thereof, wherein u is 0 or 1.

In some embodiments is a compound of Formula (Va-1), (Vb-1), (Vc-1), (Va-2), (Vb-2), or (Vc-2), or a pharmaceutically acceptable salt or solvate thereof, wherein v is 0 or 1.

In some embodiments is a compound of Formula (Va-1), (Vb-1), (Vc-1), (Va-2), (Vb-2), or (Vc-2), or a pharmaceutically acceptable salt or solvate thereof, wherein X⁵ is N(R¹).

In some embodiments is a compound of Formula (Va-1), (Vb-1), (Vc-1), (Va-2), (Vb-2), or (Vc-2), or a pharmaceutically acceptable salt or solvate thereof, wherein R¹ is hydrogen. In some embodiments is a compound of Formula (Va-1), (Vb-1), (Vc-1), (Va-2), (Vb-2), or (Vc-2), or a pharmaceutically acceptable salt or solvate thereof, wherein R¹ is -L²-R⁵.

In some embodiments is a compound of Formula (Va-1), (Vb-1), (Vc-1), (Va-2), (Vb-2), or (Vc-2), or a pharmaceutically acceptable salt or solvate thereof, wherein Y is C. In some embodiments is a compound of Formula (Va-1), (Vb-1), (Vc-1), (Va-2), (Vb-2), or (Vc-2), or a pharmaceutically acceptable salt or solvate thereof, wherein Y is N. In some embodiments is a compound of Formula (Va-1), (Vb-1), (Vc-1), (Va-2), (Vb-2), or (Vc-2), or a pharmaceutically acceptable salt or solvate thereof, wherein Y is C(O).

In some embodiments is a compound of Formula (Va-1), (Vb-1), (Vc-1), (Va-2), (Vb-2), or (Vc-2), or a pharmaceutically acceptable salt or solvate thereof, wherein X is C. In some embodiments is a compound of Formula (Va-1), (Vb-1), (Vc-1), (Va-2), (Vb-2), or (Vc-2), or a pharmaceutically acceptable salt or solvate thereof, wherein X is N.

In some embodiments is a compound of Formula (Va-1), (Vb-1), (Vc-1), (Va-2), (Vb-2), or (Vc-2), or a pharmaceutically acceptable salt or solvate thereof, wherein U is C. In some embodiments is a compound of Formula (Va-1), (Vb-1), (Vc-1), (Va-2), (Vb-2), or (Vc-2), or a pharmaceutically acceptable salt or solvate thereof, wherein U is N. In some embodiments is a compound of Formula (Va-1), (Vb-1), (Vc-1), (Va-2), (Vb-2), or (Vc-2), or a pharmaceutically acceptable salt or solvate thereof, wherein U is C(O).

In some embodiments is a compound of Formula (Va-1), (Vb-1), (Vc-1), (Va-2), (Vb-2), or (Vc-2), or a pharmaceutically acceptable salt or solvate thereof, wherein Z is C(R⁸).

In some embodiments is a compound of Formula (Va-1), (Vb-1), (Vc-1), (Va-2), (Vb-2), or (Vc-2), or a pharmaceutically acceptable salt or solvate thereof, wherein R⁸ is hydrogen.

In some embodiments is a compound of Formula (Va-1), (Vb-1), (Vc-1), (Va-2), (Vb-2), or (Vc-2), or a pharmaceutically acceptable salt or solvate thereof, wherein Z is N.

In some embodiments is a compound of Formula (Va-1), (Vb-1), (Vc-1), (Va-2), (Vb-2), or (Vc-2), or a pharmaceutically acceptable salt or solvate thereof, wherein V is C(R¹⁶). In some embodiments is a compound of Formula (Va-1), (Vb-1), (Vc-1), (Va-2), (Vb-2), or (Vc-2), or a pharmaceutically acceptable salt or solvate thereof, wherein is C(H). In some embodiments is a compound of Formula (Va-1), (Vb-1), (Vc-1), (Va-2), (Vb-2), or (Vc-2), or a pharmaceutically acceptable salt or solvate thereof, wherein V is N.

In some embodiments is a compound of Formula (Va-1), (Vb-1), (Vc-1), (Va-2), (Vb-2), or (Vc-2), or a pharmaceutically acceptable salt or solvate thereof, wherein J is C(R¹⁷).

In some embodiments is a compound of Formula (Va-1), (Vb-1), (Vc-1), (Va-2), (Vb-2), or (Vc-2), or a pharmaceutically acceptable salt or solvate thereof, wherein W is C(R¹⁸). In some embodiments is a compound of Formula (Va-1), (Vb-1), (Vc-1), (Va-2), (Vb-2), or (Vc-2), or a pharmaceutically acceptable salt or solvate thereof, wherein W is C(H). In some embodiments is a compound of Formula (Va-1), (Vb-1), (Vc-1), (Va-2), (Vb-2), or (Vc-2), or a pharmaceutically acceptable salt or solvate thereof, wherein W is N.

In some embodiments is a compound of Formula (Va-1), (Vb-1), (Vc-1), (Va-2), (Vb-2), or (Vc-2), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (Va-1), (Vb-1), (Vc-1), (Va-2), (Vb-2), or (Vc-2), or a pharmaceutically acceptable salt or solvate thereof, wherein R² is selected from —OR¹², —SR¹², and C₁₋₆alkyl, wherein C₁₋₆alkyl is optionally substituted with one, two, or three R²⁰%. In some embodiments is a compound of Formula (Va-1), (Vb-1), (Vc-1), (Va-2), (Vb-2), or (Vc-2), or a pharmaceutically acceptable salt or solvate thereof, wherein R² is —OR¹².

In some embodiments is a compound of Formula (Va-1), (Vb-1), (Vc-1), (Va-2), (Vb-2), or (Vc-2), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (Va), (Vb), or (Vc), or a pharmaceutically acceptable salt or solvate thereof, wherein R¹² is C₁₋₆alkyl optionally substituted with one, two, or three R^{20d}. In some embodiments is a compound of Formula (Va-1), (Vb-1), (Vc-1), (Va-2), (Vb-2), or (Vc-2), or a pharmaceutically acceptable salt or solvate thereof, wherein R¹² is C₂₋₉heterocycloalkyl optionally substituted with one, two, or three R^{20d}. In some embodiments is a compound of Formula (Va-1), (Vb-1), (Vc-1), (Va-2), (Vb-2), or (Vc-2), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (Va-1), (Vb-1), (Vc-1), (Va-2), (Vb-2), or (Vc-2), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (Va-1), (Vb-1), (Vc-1), (Va-2), (Vb-2), or (Vc-2), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (Va-1), (Vb-1), (Vc-1), (Va-2), (Vb-2), or (Vc-2), or a pharmaceutically acceptable salt or solvate thereof, wherein R² is selected from

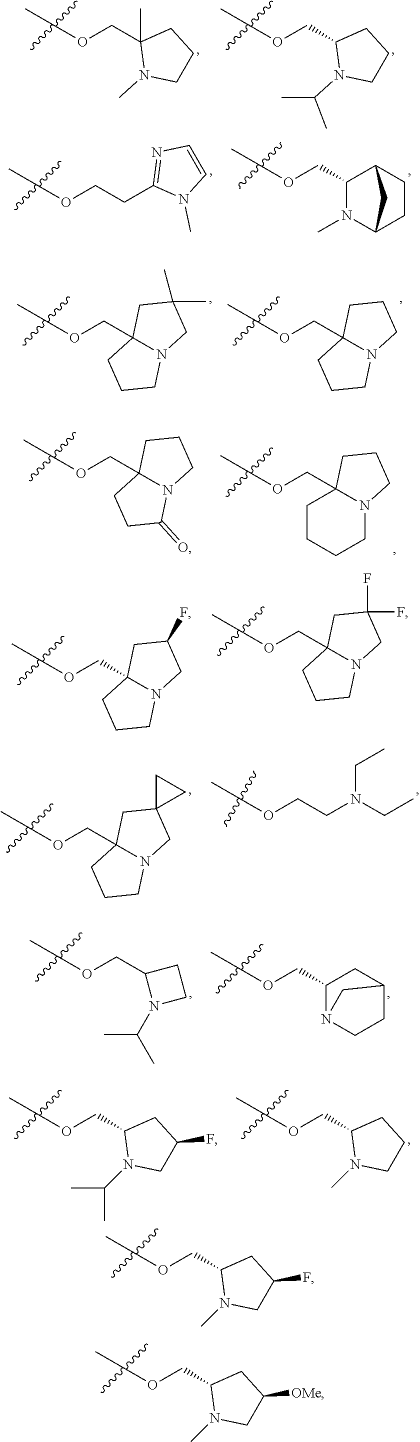

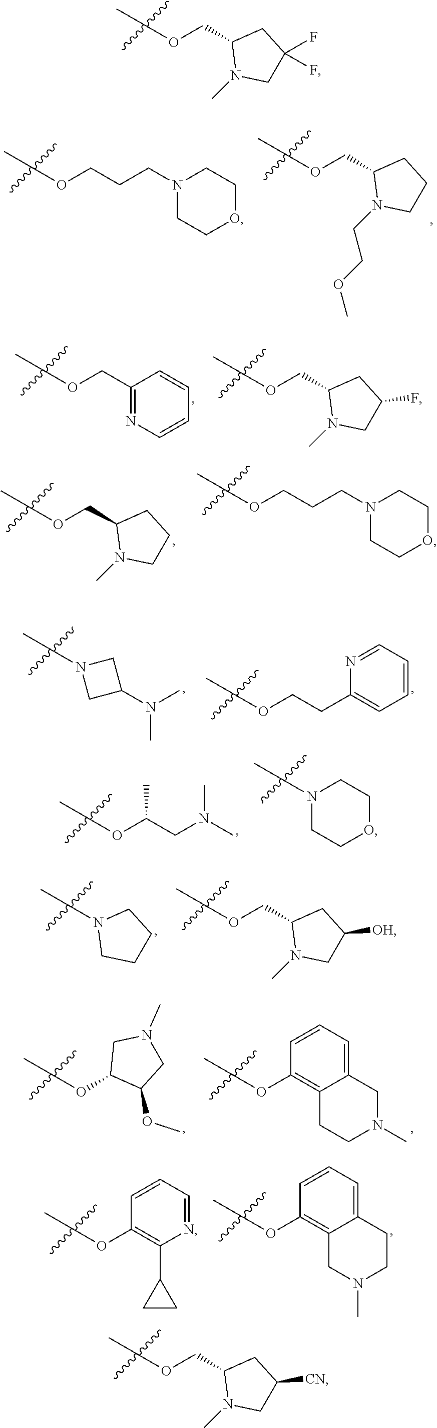

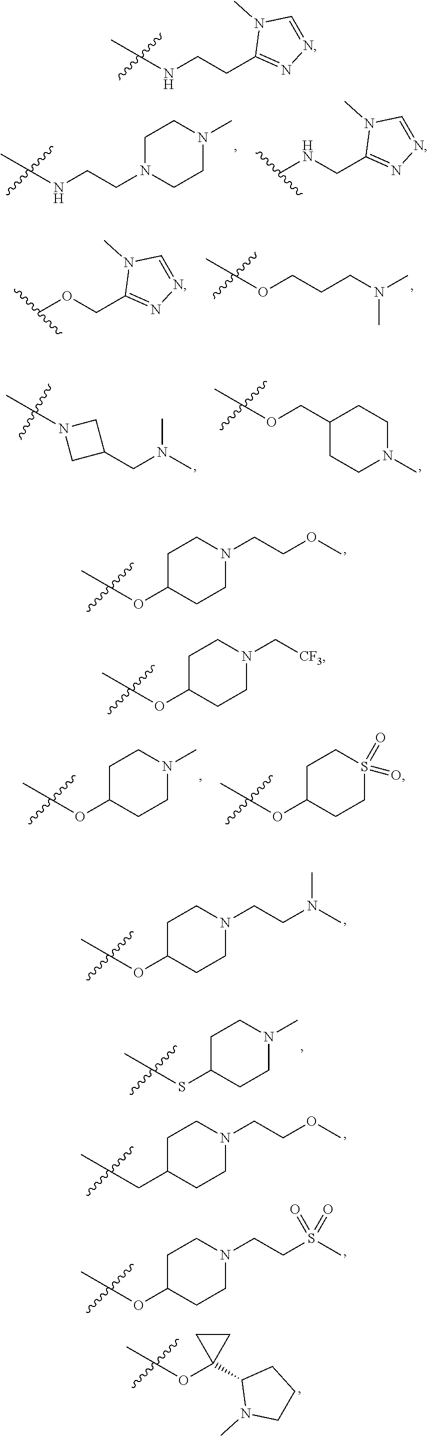

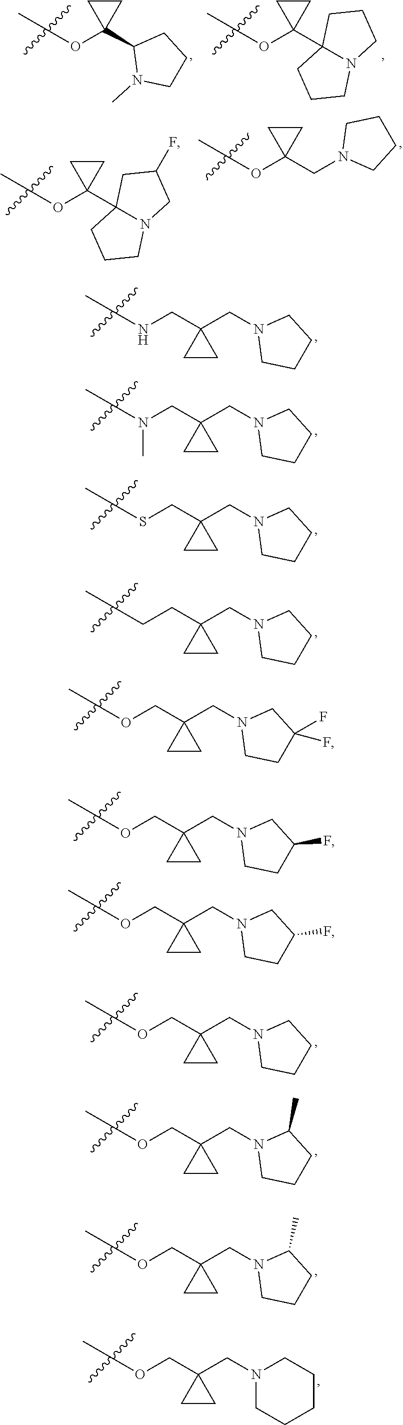

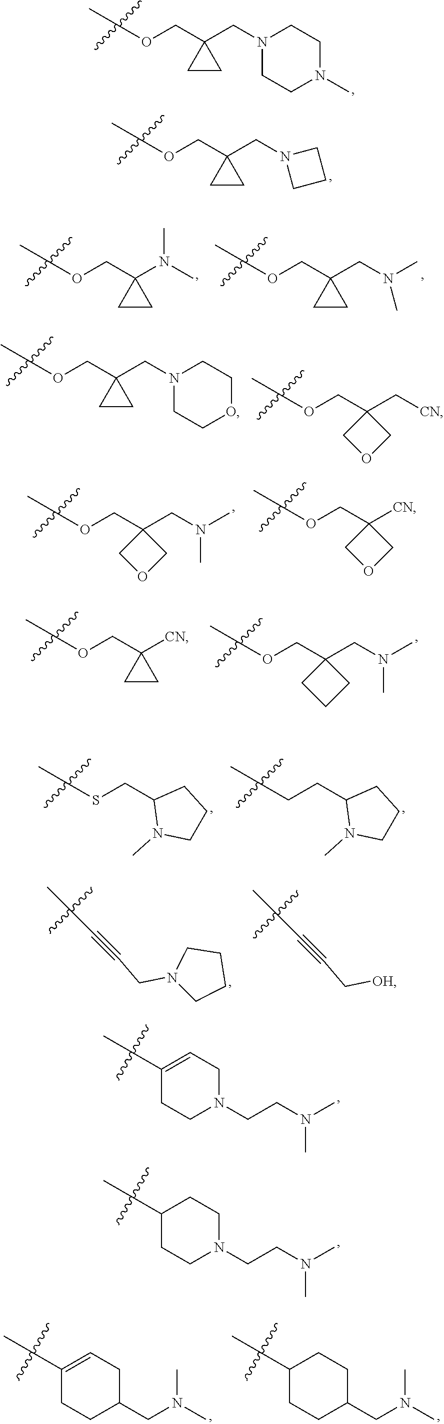

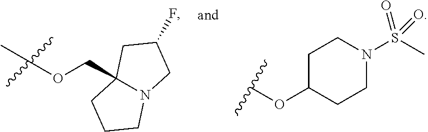 [SMILES: *OC[C@]12CCCN1C[C@@H](F)C2 ; *OC1CCN(S(C)(=O)=O)CC1]

In some embodiments is a compound of Formula (Va-1), (Vb-1), (Vc-1), (Va-2), (Vb-2), or (Vc-2), or a pharmaceutically acceptable salt or solvate thereof, wherein L¹ is a bond. In some embodiments is a compound of Formula (Va-1), (Vb-1), (Vc-1), (Va-2), (Vb-2), or (Vc-2), or a pharmaceutically acceptable salt or solvate thereof, wherein L¹ is O.

In some embodiments is a compound of Formula (Va-1), (Vb-1), (Vc-1), (Va-2), (Vb-2), or (Vc-2), or a pharmaceutically acceptable salt or solvate thereof, wherein R¹⁹ is C₁₋₉heteroaryl optionally substituted with one, two, or three R²⁰ⁱ. In some embodiments is a compound of Formula (Va-1), (Vb-1), (Vc-1), (Va-2), (Vb-2), or (Vc-2), or a pharmaceutically acceptable salt or solvate thereof, wherein R¹⁹ is C₆₋₁₀aryl optionally substituted with one, two, or three R²⁰ⁱ.

In some embodiments is a compound of Formula (Va-1), (Vb-1), (Vc-1), (Va-2), (Vb-2), or (Vc-2), or a pharmaceutically acceptable salt or solvate thereof, wherein L² is selected from a bond, C₁-C₆alkyl, and —C(O)—. In some embodiments is a compound of Formula (Va-1), (Vb-1), (Vc-1), (Va-2), (Vb-2), or (Vc-2), or a pharmaceutically acceptable salt or solvate thereof, wherein L² is a bond. In some embodiments is a compound of Formula (Va-1), (Vb-1), (Vc-1), (Va-2), (Vb-2), or (Vc-2), or a pharmaceutically acceptable salt or solvate thereof, wherein L² is, C₁-C₆alkyl.

In some embodiments is a compound of Formula (Va-1), (Vb-1), or (Vc-1), or a pharmaceutically acceptable salt or solvate thereof, wherein each R⁵ is independently hydrogen. In some embodiments is a compound of Formula (Va-1), (Vb-1), or (Vc-1), or a pharmaceutically acceptable salt or solvate thereof, wherein each R⁵ is independently a group other than an electrophilic moiety capable of forming a covalent bond with the cysteine residue at position 12 of a KRAS protein. In some embodiments is a compound of Formula (Va-1), (Vb-1), or (Vc-1), or a pharmaceutically acceptable salt or solvate thereof, wherein each R⁵ is independently —H, —NH₂, —OH, —NH(C₁₋₆ alkyl),

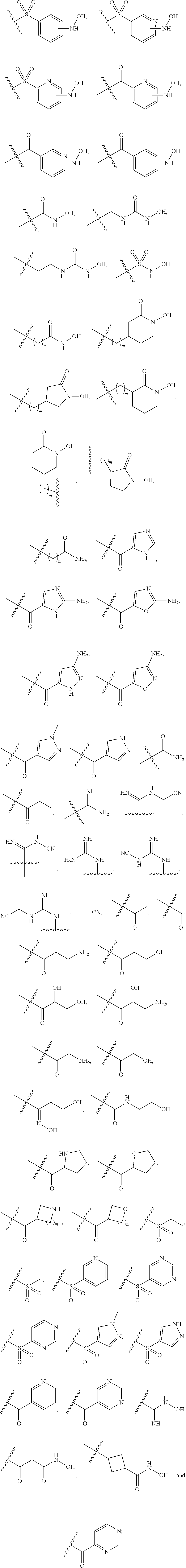

and m, when present, is 0, 1, 2, or 3.

In some embodiments of the subject compound of Formula (Va-1), (Vb-1), or (Vc-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C_{2- 9}heterocycloalkyl, C₆₋₁₀aryl, and C₁₋₉heteroaryl are optionally substituted with one, two, or three R^{20a}. In some embodiments of the subject compound of Formula (Va-1), (Vb-1), or (Vc-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (Va-1), (Vb-1), or (Vc-1), or a pharmaceutically acceptable salt or solvate thereof, each R⁵ is independently halogen. In further embodiments of the subject compound of Formula (Va-1), (Vb-1), or (Vc-1), or a pharmaceutically acceptable salt or solvate thereof, each R⁵ is independently —CN. In embodiments of the subject compound of Formula (Va-1), (Vb-1), or (Vc-1), or a pharmaceutically acceptable salt or solvate thereof, each R⁵ is independently —OH. In some embodiments of the subject compound of Formula (Va-1), (Vb-1), or (Vc-1), or a pharmaceutically acceptable salt or solvate thereof, each R⁵ is independently —NH₂. In further embodiments of the subject compound of Formula (Va-1), (Vb-1), or (Vc-1), or a pharmaceutically acceptable salt or solvate thereof, each R⁵ is independently —C(O)OH. In select embodiments of the subject compound of Formula (Va-1), (Vb-1), or (Vc-1), or a pharmaceutically acceptable salt or solvate thereof, each R⁵ is independently —OC(O)NH₂. In embodiments of the subject compound of Formula (Va-1), (Vb-1), or (Vc-1), or a pharmaceutically acceptable salt or solvate thereof, each R⁵ is independently —NHC(O)NH₂. In embodiments of the subject compound of Formula (Va-1), (Vb-1), or (Vc-1), or a pharmaceutically acceptable salt or solvate thereof, each R⁵ is independently —NHC(O)OH. In some embodiments of the subject compound of Formula (Va-1), (Vb-1), or (Vc-1), or a pharmaceutically acceptable salt or solvate thereof, each R⁵ is independently —NHS(O)₂H. In embodiments of the subject compound of Formula (Va-1), (Vb-1), or (Vc-1), or a pharmaceutically acceptable salt or solvate thereof, each R⁵ is independently —C(O)NH₂. In some embodiments of the subject compound of Formula (Va-1), (Vb-1), or (Vc-1), or a pharmaceutically acceptable salt or solvate thereof, each R⁵ is independently each R⁵ is independently hydrogen. In embodiments of the subject compound of Formula (Va-1), (Vb-1), or (Vc-1), or a pharmaceutically acceptable salt or solvate thereof, each R⁵ is independently oxo. In further embodiments of the subject compound of Formula (Va-1), (Vb-1), or (Vc-1), or a pharmaceutically acceptable salt or solvate thereof, each R⁵ is independently C₁₋₆alkyl optionally substituted with one, two, or three R^{20a}. In select embodiments of the subject compound of Formula (Va-1), (Vb-1), or (Vc-1), or a pharmaceutically acceptable salt or solvate thereof, each R⁵ is independently C₃₋₆cycloalkyl optionally substituted with one, two, or three R^{20a}. In embodiments of the subject compound of Formula (Va-1), (Vb-1), or (Vc-1), or a pharmaceutically acceptable salt or solvate thereof, each R⁵ is independently C₂₋₉heterocycloalkyl optionally substituted with one, two, or three R^{20a}. In some embodiments of the subject compound of Formula (Va-1), (Vb-1), or (Vc-1), or a pharmaceutically acceptable salt or solvate thereof, each R⁵ is independently C₆₋₁₀aryl optionally substituted with one, two, or three R^{20a}. In further embodiments of the subject compound of Formula (Va-1), (Vb-1), or (Vc-1), or a pharmaceutically acceptable salt or solvate thereof, each R⁵ is independently each R⁵ is independently C₁₋₉heteroaryl optionally substituted with one, two, or three R^{20a}. In select embodiments of the subject compound of Formula (Va-1), (Vb-1), or (Vc-1), or a pharmaceutically acceptable salt or solvate thereof, each R⁵ is independently —OR¹². In embodiments of the subject compound of Formula (Va-1), (Vb-1), or (Vc-1), or a pharmaceutically acceptable salt or solvate thereof, each R⁵ is independently —N(R¹²)(R¹³). In select embodiments of the subject compound of Formula (Va-1), (Vb-1), or (Vc-1), or a pharmaceutically acceptable salt or solvate thereof, each R⁵ is independently N(R¹⁴)S(O)₂R¹⁵. In select embodiments of the subject compound of Formula (Va-1), (Vb-1), or (Vc-1), or a pharmaceutically acceptable salt or solvate thereof, each R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (Va-2), (Vb-2), or (Vc-2), or a pharmaceutically acceptable salt or solvate thereof, wherein each R⁵ is independently an electrophilic moiety capable of forming a covalent bond with a residue at position 12 of a KRAS protein, e.g., the cysteine residue at position 12. In some embodiments is a compound of Formula (Va-2), (Vb-2), or (Vc-2), or a pharmaceutically acceptable salt or solvate thereof, wherein each R⁵ is independently an electrophilic moiety capable of forming a covalent bond with the cysteine residue at position 12 of a KRAS protein. In some embodiments is a compound of Formula (Va-2), (Vb-2), or (Vc-2), or a pharmaceutically acceptable salt or solvate thereof, wherein each R⁵ is independently selected from the group consisting of

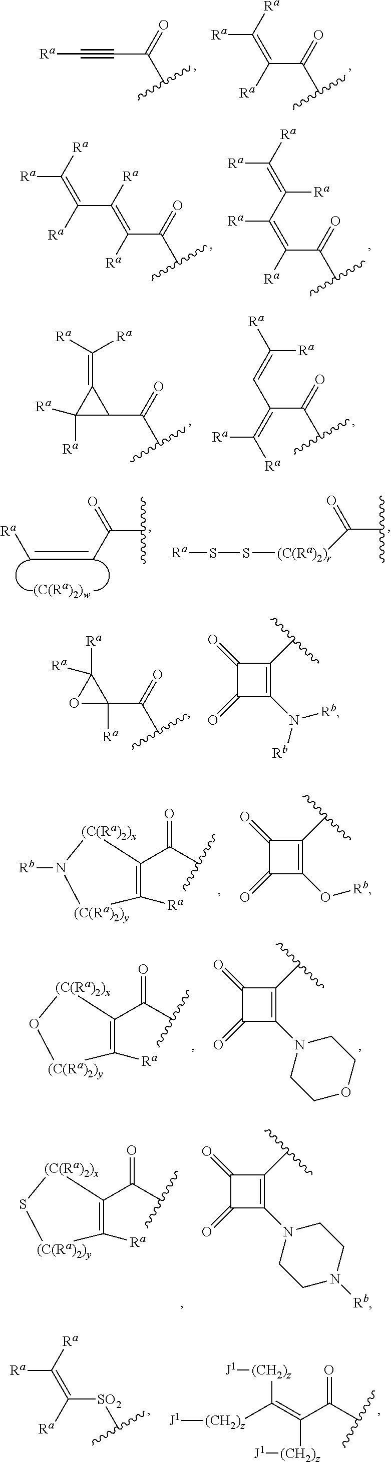

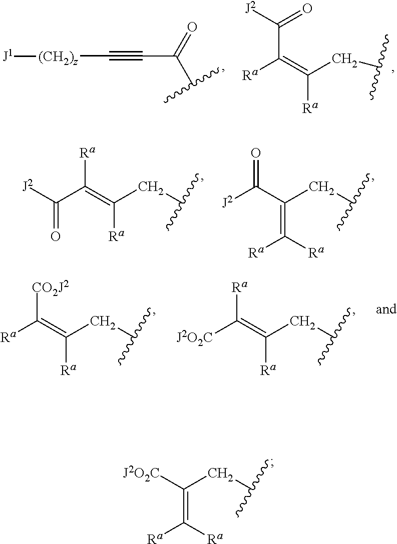

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-(C(R^{b})₂)ᵣ—, (R^{a})(R^{c})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{e} is —NR^{b}R^{b} or —OR^{b}; R^{d} and R^{e} are each, independently, —(C(R^{b})₂)—NR^{b}R^{b}, or —(C(R^{b})₂), —OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

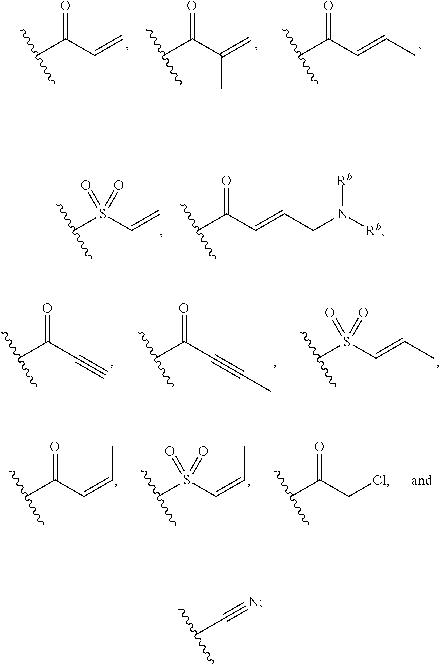

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In some embodiments is a compound of Formula (I-2), (I′), (I″-2), (Ia), (Ib), (Ic), (Id), (Ie), (If), (Ig), (II-2), (IIa), (IIb), (IIc′), (IIc), (IId′), (IId), (IIe), (IIf), (IIg), (IIh), (IIi), (IIj), (IIk), (IIm), (IIIa-2), (IIIb-2), (IIIc-2), (IIId-2), (IIIe-2), (IIIf-2), (IIIg-2), (IIIh-2), (IIIi-2), (IIIa-4), (IIIb-4), (IIIc-4), (IIId-4), (IIIe-4), (IIIf-4), (IIIg-4), (IIIh-4), (IIIi-4), (IVa-2), (IVb-2), (IVc-2), (Va-2), (Vb-2), or (Vc-2), or a pharmaceutically acceptable salt or solvate thereof, wherein R⁵ is an electrophilic moiety capable of forming a covalent bond with a residue at position 12 of a KRAS protein, e.g., the cysteine residue at position 12. In some embodiments is a compound of Formula (I-2), (I′), (I″-2), (Ia), (Ib), (Ic), (Id), (Ie), (If), (Ig), (II-2), (IIa), (IIb), (IIc′), (IIc), (IId′), (IId), (IIe), (IIf), (IIg), (IIh), (IIi), (IIj), (IIk), (IIm), (IIIa-2), (IIIb-2), (IIIc-2), (IIId-2), (IIIe-2), (IIIf-2), (IIIg-2), (IIIh-2), (IIIi-2), (IIIa-4), (IIIb-4), (IIIc-4), (IIId-4), (IIIe-4), (IIIf-4), (IIIg-4), (IIIh-4), (IIIi-4), (IVa-2), (IVb-2), (IVc-2), (Va-2), (Vb-2), or (Vc-2), or a pharmaceutically acceptable salt or solvate thereof, wherein R⁵ is an electrophilic moiety capable of forming a covalent bond with the cysteine residue at position 12 of a KRAS protein. In some embodiments is a compound of Formula (I-2), (I′), (I″-2), (Ia), (Ib), (Ic), (Id), (Ie), (If), (Ig), (II-2), (IIa), (IIb), (IIc′), (IIc), (IId′), (IId), (IIe), (IIf), (IIg), (IIh), (IIi), (IIj), (IIk), (IIm), (IIIa-2), (IIIb-2), (IIIc-2), (IIId-2), (IIIe-2), (IIIf-2), (IIIg-2), (IIIh-2), (IIIi-2), (IIIa-4), (IIIb-4), (IIIc-4), (IIId-4), (IIIe-4), (IIIf-4), (IIIg-4), (IIIh-4), (IIIi-4), (IVa-2), (IVb-2), (IVc-2), (Va-2), (Vb-2), or (Vc-2), or a pharmaceutically acceptable salt or solvate thereof, wherein R⁵ is selected from the group consisting of

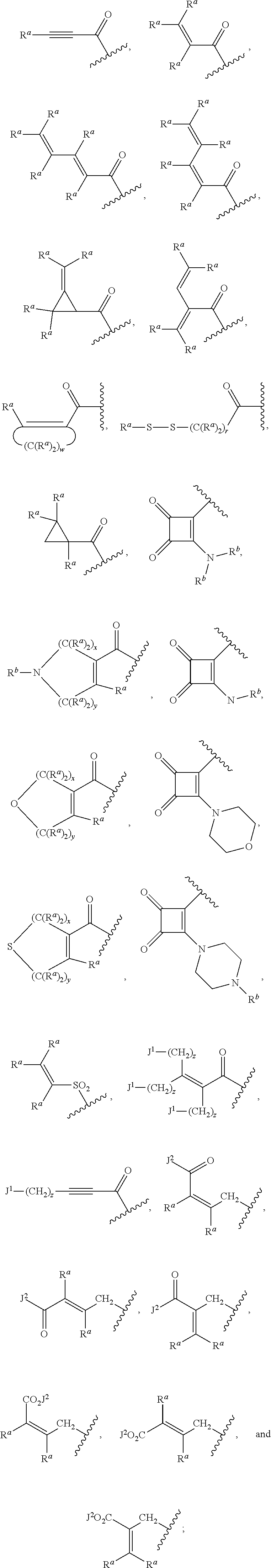

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-(C(R^{b})₂)ᵣ—, (R^{d})(R⁶)CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, R⁵ is selected from the group consisting of:

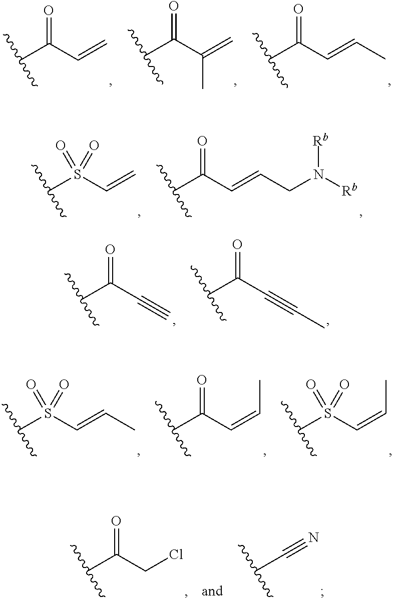

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In some embodiments, the disclosure provides a compound selected from:

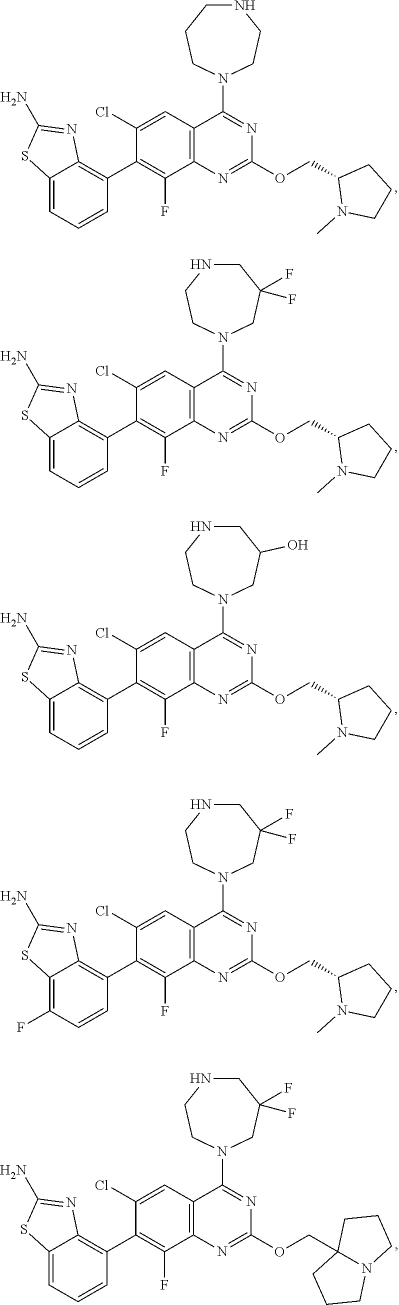 [SMILES: CN1CCC[C@H]1COc1nc(N2CCCNCC2)c2cc(Cl)c(-c3cccc4sc(N)nc34)c(F)c2n1 ; CN1CCC[C@H]1COc1nc(N2CCNCC(F)(F)C2)c2cc(Cl)c(-c3cccc4sc(N)nc34)c(F)c2n1 ; CN1CCC[C@H]1COc1nc(N2CCNCC(O)C2)c2cc(Cl)c(-c3cccc4sc(N)nc34)c(F)c2n1 ; CN1CCC[C@H]1COc1nc(N2CCNCC(F)(F)C2)c2cc(Cl)c(-c3ccc(F)c4sc(N)nc34)c(F)c2n1 ; Nc1nc2c(-c3c(Cl)cc4c(N5CCNCC(F)(F)C5)nc(OCC56CCCN5CCC6)nc4c3F)cccc2s1]

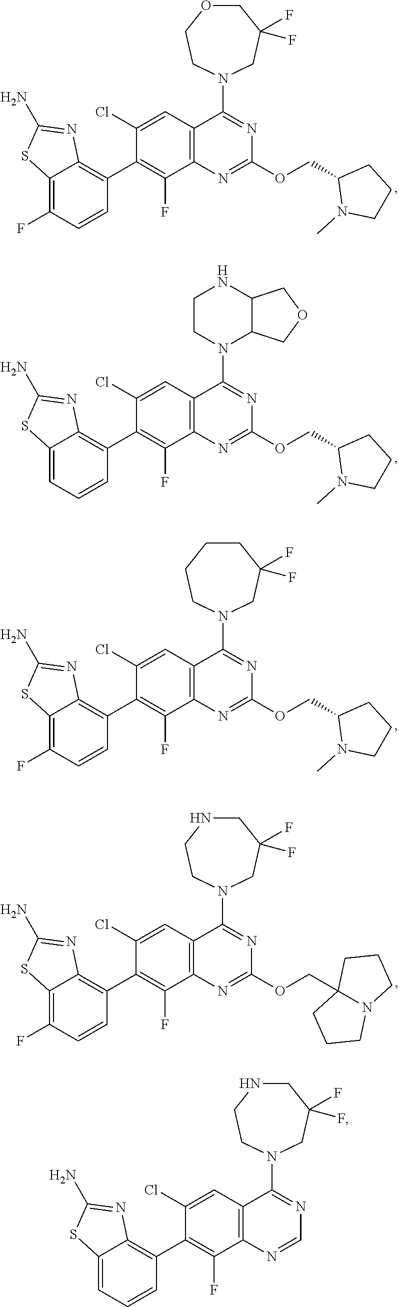 [SMILES: CN1CCC[C@H]1COc1nc(N2CCOCC(F)(F)C2)c2cc(Cl)c(-c3ccc(F)c4sc(N)nc34)c(F)c2n1 ; CN1CCC[C@H]1COc1nc(N2CCNC3COCC32)c2cc(Cl)c(-c3cccc4sc(N)nc34)c(F)c2n1 ; CN1CCC[C@H]1COc1nc(N2CCCCC(F)(F)C2)c2cc(Cl)c(-c3ccc(F)c4sc(N)nc34)c(F)c2n1 ; Nc1nc2c(-c3c(Cl)cc4c(N5CCNCC(F)(F)C5)nc(OCC56CCCN5CCC6)nc4c3F)ccc(F)c2s1 ; Nc1nc2c(-c3c(Cl)cc4c(N5CCNCC(F)(F)C5)ncnc4c3F)cccc2s1]

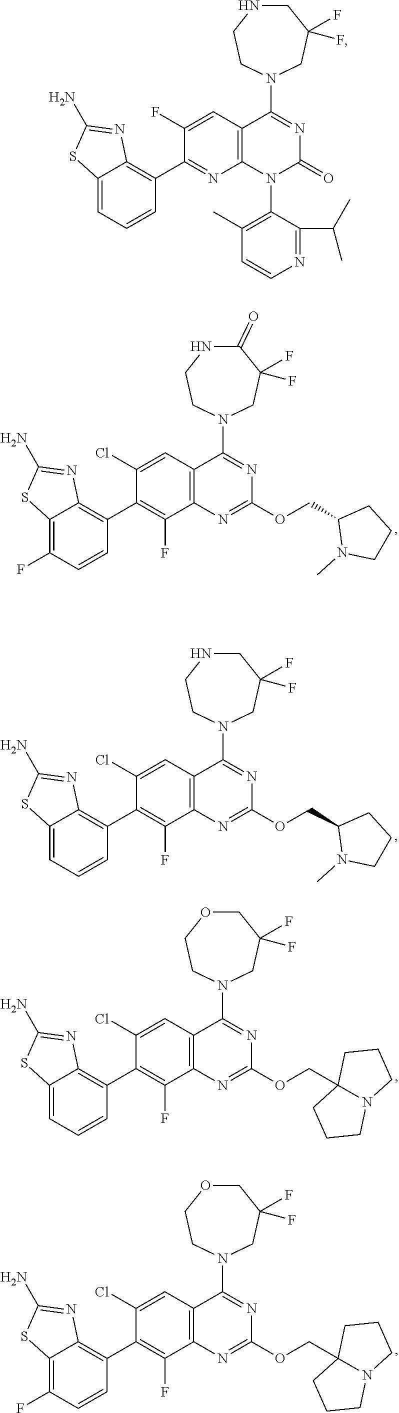

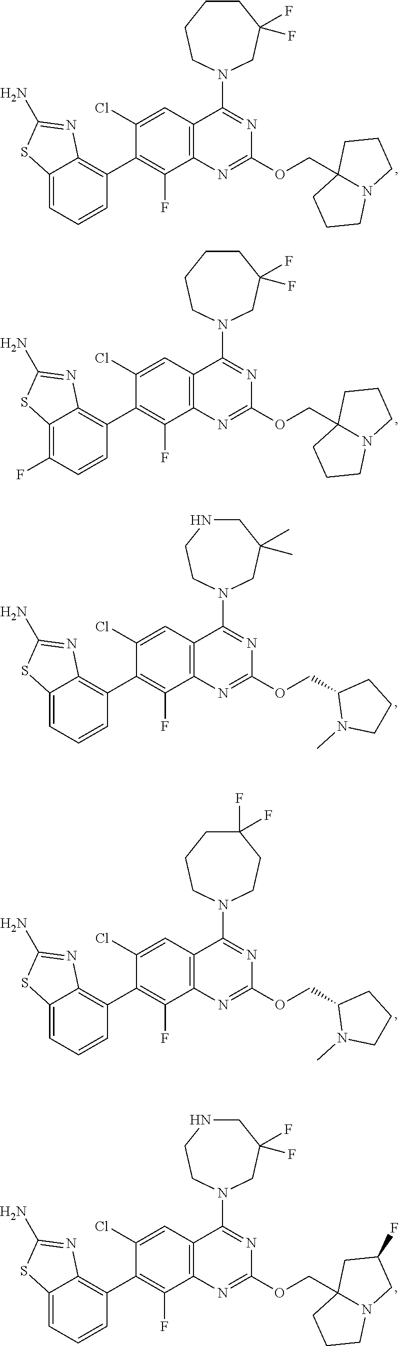 [SMILES: Nc1nc2c(-c3c(Cl)cc4c(N5CCCCC(F)(F)C5)nc(OCC56CCCN5CCC6)nc4c3F)cccc2s1 ; Nc1nc2c(-c3c(Cl)cc4c(N5CCCCC(F)(F)C5)nc(OCC56CCCN5CCC6)nc4c3F)ccc(F)c2s1 ; CN1CCC[C@H]1COc1nc(N2CCNCC(C)(C)C2)c2cc(Cl)c(-c3cccc4sc(N)nc34)c(F)c2n1 ; CN1CCC[C@H]1COc1nc(N2CCCC(F)(F)CC2)c2cc(Cl)c(-c3cccc4sc(N)nc34)c(F)c2n1 ; Nc1nc2c(-c3c(Cl)cc4c(N5CCNCC(F)(F)C5)nc(OCC56CCCN5C[C@H](F)C6)nc4c3F)cccc2s1]

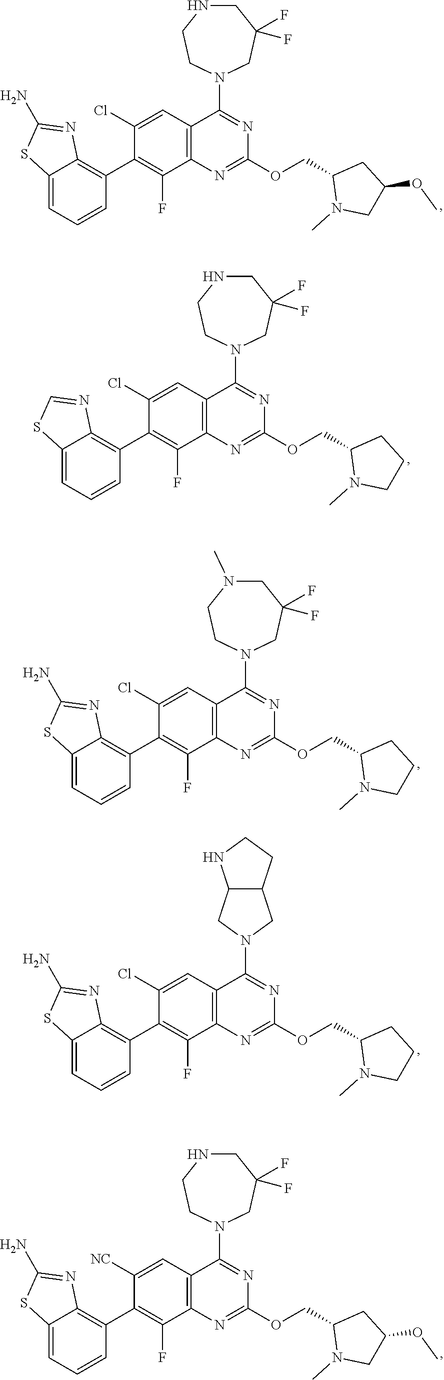 [SMILES: CO[C@@H]1C[C@@H](COc2nc(N3CCNCC(F)(F)C3)c3cc(Cl)c(-c4cccc5sc(N)nc45)c(F)c3n2)N(C)C1 ; CN1CCC[C@H]1COc1nc(N2CCNCC(F)(F)C2)c2cc(Cl)c(-c3cccc4scnc34)c(F)c2n1 ; CN1CCN(c2nc(OC[C@@H]3CCCN3C)nc3c(F)c(-c4cccc5sc(N)nc45)c(Cl)cc23)CC(F)(F)C1 ; CN1CCC[C@H]1COc1nc(N2CC3CCNC3C2)c2cc(Cl)c(-c3cccc4sc(N)nc34)c(F)c2n1 ; CO[C@H]1C[C@@H](COc2nc(N3CCNCC(F)(F)C3)c3cc(C#N)c(-c4cccc5sc(N)nc45)c(F)c3n2)N(C)C1]

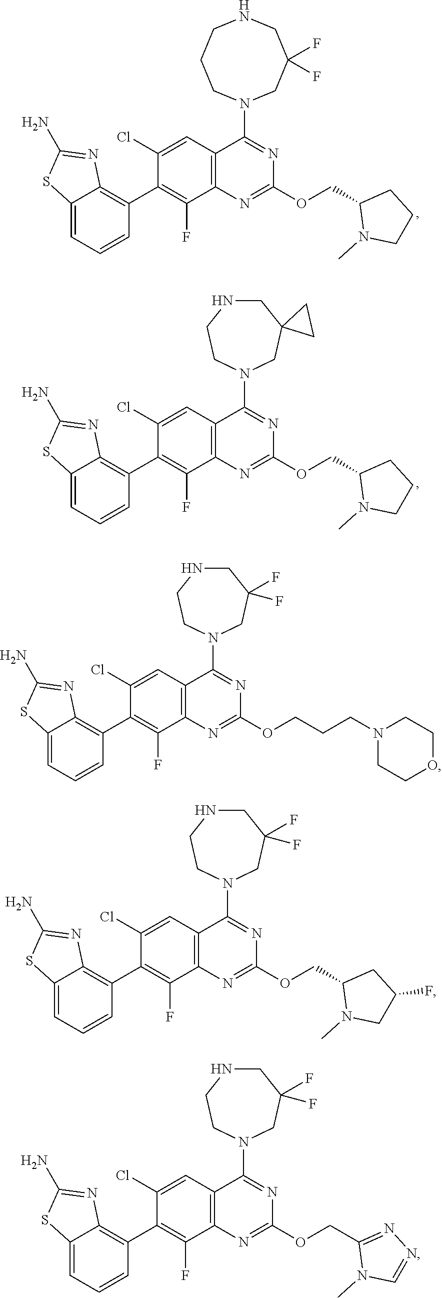

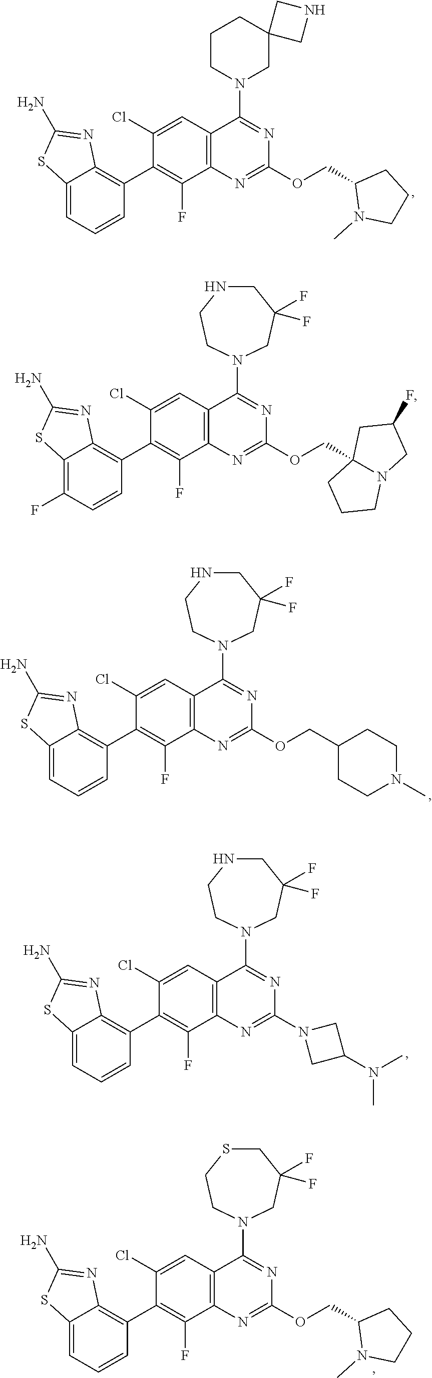 [SMILES: CN1CCC[C@H]1COc1nc(N2CCCC3(CNC3)C2)c2cc(Cl)c(-c3cccc4sc(N)nc34)c(F)c2n1 ; Nc1nc2c(-c3c(Cl)cc4c(N5CCNCC(F)(F)C5)nc(OC[C@@]56CCCN5C[C@H](F)C6)nc4c3F)ccc(F)c2s1 ; CN1CCC(COc2nc(N3CCNCC(F)(F)C3)c3cc(Cl)c(-c4cccc5sc(N)nc45)c(F)c3n2)CC1 ; CN(C)C1CN(c2nc(N3CCNCC(F)(F)C3)c3cc(Cl)c(-c4cccc5sc(N)nc45)c(F)c3n2)C1 ; CN1CCC[C@H]1COc1nc(N2CCSCC(F)(F)C2)c2cc(Cl)c(-c3cccc4sc(N)nc34)c(F)c2n1]

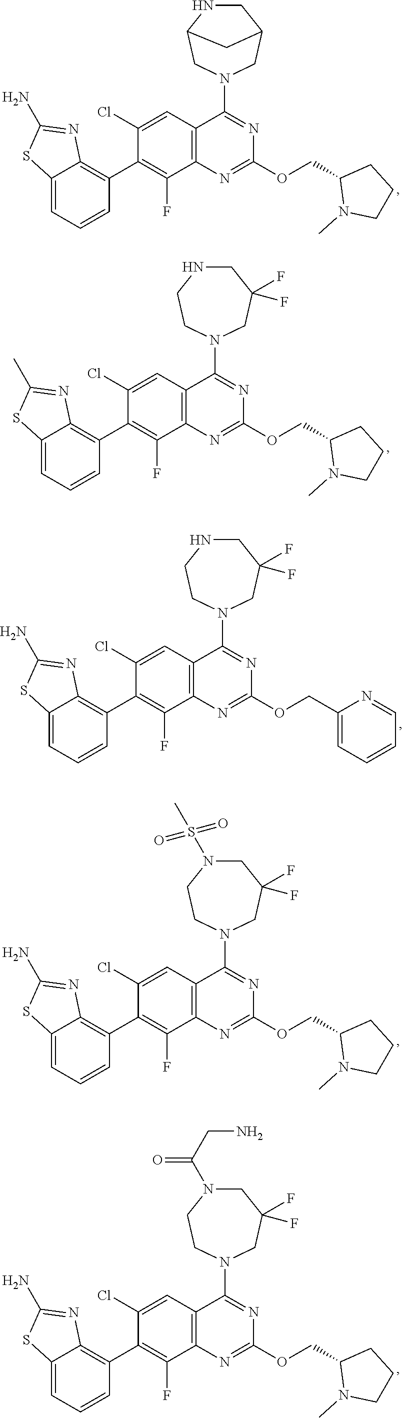 [SMILES: CN1CCC[C@H]1COc1nc(N2CC3CNC(C3)C2)c2cc(Cl)c(-c3cccc4sc(N)nc34)c(F)c2n1 ; Cc1nc2c(-c3c(Cl)cc4c(N5CCNCC(F)(F)C5)nc(OC[C@@H]5CCCN5C)nc4c3F)cccc2s1 ; Nc1nc2c(-c3c(Cl)cc4c(N5CCNCC(F)(F)C5)nc(OCc5ccccn5)nc4c3F)cccc2s1 ; CN1CCC[C@H]1COc1nc(N2CCN(S(C)(=O)=O)CC(F)(F)C2)c2cc(Cl)c(-c3cccc4sc(N)nc34)c(F)c2n1 ; CN1CCC[C@H]1COc1nc(N2CCN(C(=O)CN)CC(F)(F)C2)c2cc(Cl)c(-c3cccc4sc(N)nc34)c(F)c2n1]

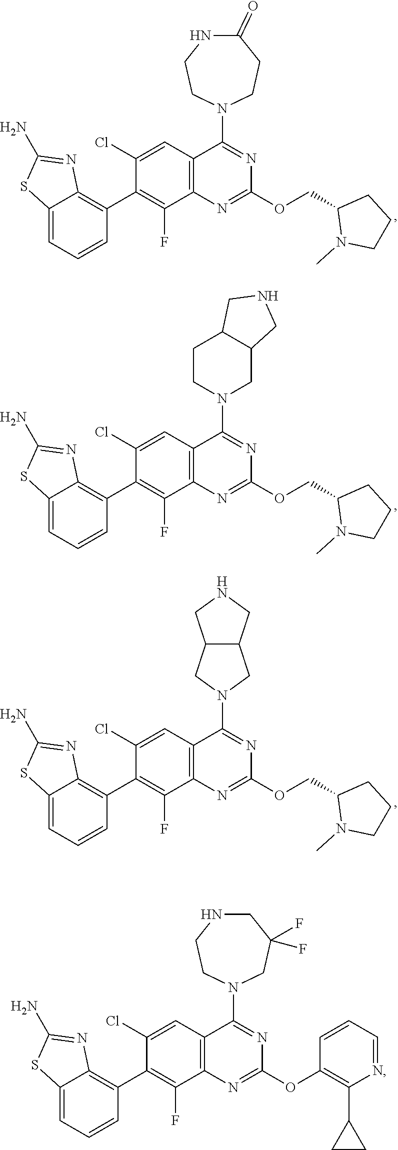 [SMILES: CN1CCC[C@H]1COc1nc(N2CCNC(=O)CC2)c2cc(Cl)c(-c3cccc4sc(N)nc34)c(F)c2n1 ; CN1CCC[C@H]1COc1nc(N2CCC3CNCC3C2)c2cc(Cl)c(-c3cccc4sc(N)nc34)c(F)c2n1 ; CN1CCC[C@H]1COc1nc(N2CC3CNCC3C2)c2cc(Cl)c(-c3cccc4sc(N)nc34)c(F)c2n1 ; Nc1nc2c(-c3c(Cl)cc4c(N5CCNCC(F)(F)C5)nc(Oc5cccnc5C5CC5)nc4c3F)cccc2s1]

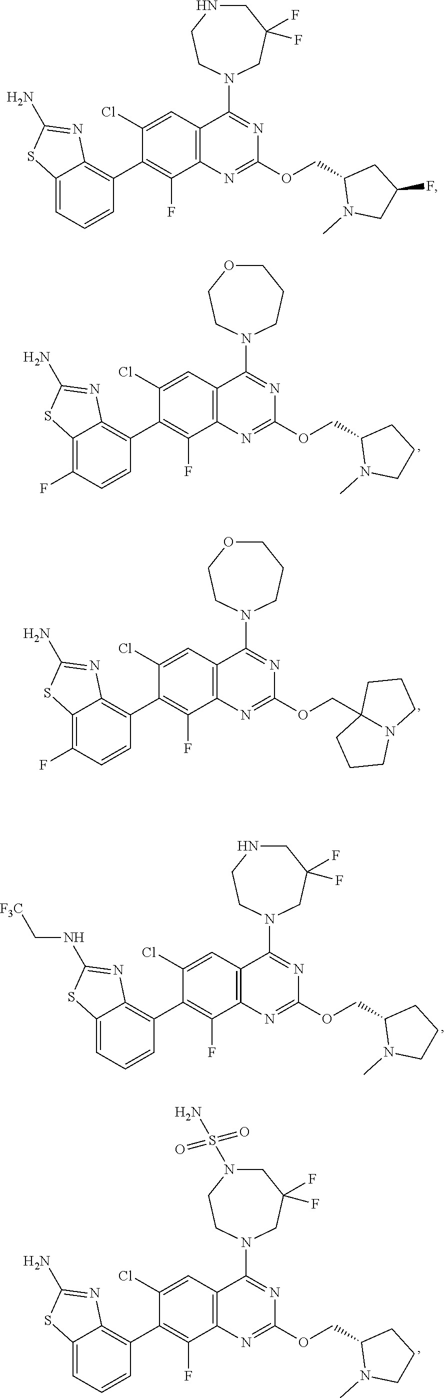 [SMILES: CN1C[C@H](F)C[C@H]1COc1nc(N2CCNCC(F)(F)C2)c2cc(Cl)c(-c3cccc4sc(N)nc34)c(F)c2n1 ; CN1CCC[C@H]1COc1nc(N2CCCOCC2)c2cc(Cl)c(-c3ccc(F)c4sc(N)nc34)c(F)c2n1 ; Nc1nc2c(-c3c(Cl)cc4c(N5CCCOCC5)nc(OCC56CCCN5CCC6)nc4c3F)ccc(F)c2s1 ; CN1CCC[C@H]1COc1nc(N2CCNCC(F)(F)C2)c2cc(Cl)c(-c3cccc4sc(NCC(F)(F)F)nc34)c(F)c2n1 ; CN1CCC[C@H]1COc1nc(N2CCN(S(N)(=O)=O)CC(F)(F)C2)c2cc(Cl)c(-c3cccc4sc(N)nc34)c(F)c2n1]

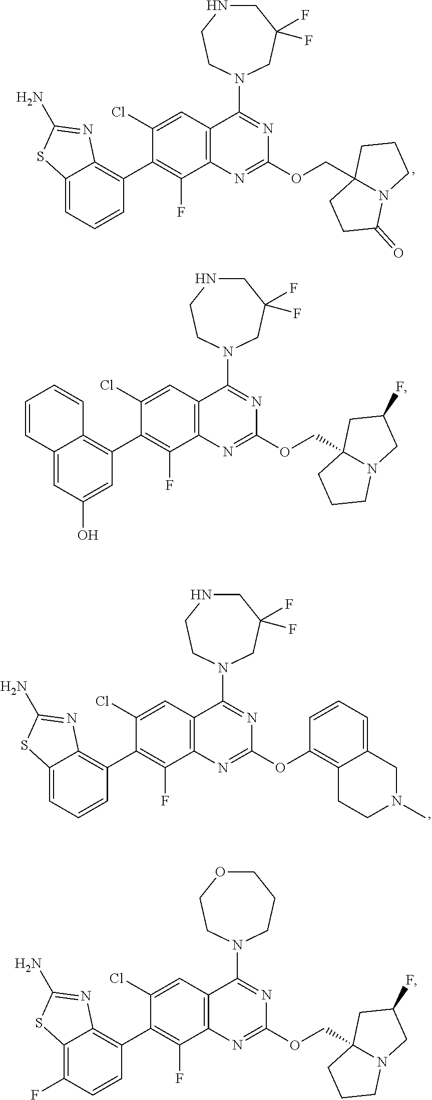 [SMILES: Nc1nc2c(-c3c(Cl)cc4c(N5CCNCC(F)(F)C5)nc(OCC56CCCN5C(=O)CC6)nc4c3F)cccc2s1 ; Oc1cc(-c2c(Cl)cc3c(N4CCNCC(F)(F)C4)nc(OC[C@@]45CCCN4C[C@H](F)C5)nc3c2F)c2ccccc2c1 ; CN1CCc2c(cccc2Oc2nc(N3CCNCC(F)(F)C3)c3cc(Cl)c(-c4cccc5sc(N)nc45)c(F)c3n2)C1 ; Nc1nc2c(-c3c(Cl)cc4c(N5CCCOCC5)nc(OC[C@@]56CCCN5C[C@H](F)C6)nc4c3F)ccc(F)c2s1]

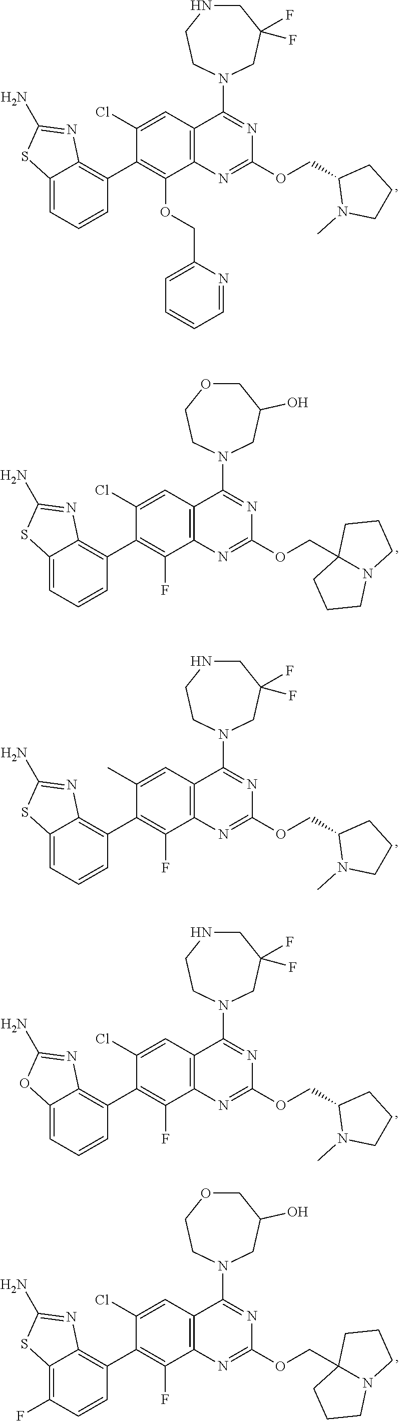 [SMILES: CN1CCC[C@H]1COc1nc(N2CCNCC(F)(F)C2)c2cc(Cl)c(-c3cccc4sc(N)nc34)c(OCc3ccccn3)c2n1 ; Nc1nc2c(-c3c(Cl)cc4c(N5CCOCC(O)C5)nc(OCC56CCCN5CCC6)nc4c3F)cccc2s1 ; Cc1cc2c(N3CCNCC(F)(F)C3)nc(OC[C@@H]3CCCN3C)nc2c(F)c1-c1cccc2sc(N)nc12 ; CN1CCC[C@H]1COc1nc(N2CCNCC(F)(F)C2)c2cc(Cl)c(-c3cccc4oc(N)nc34)c(F)c2n1 ; Nc1nc2c(-c3c(Cl)cc4c(N5CCOCC(O)C5)nc(OCC56CCCN5CCC6)nc4c3F)ccc(F)c2s1]

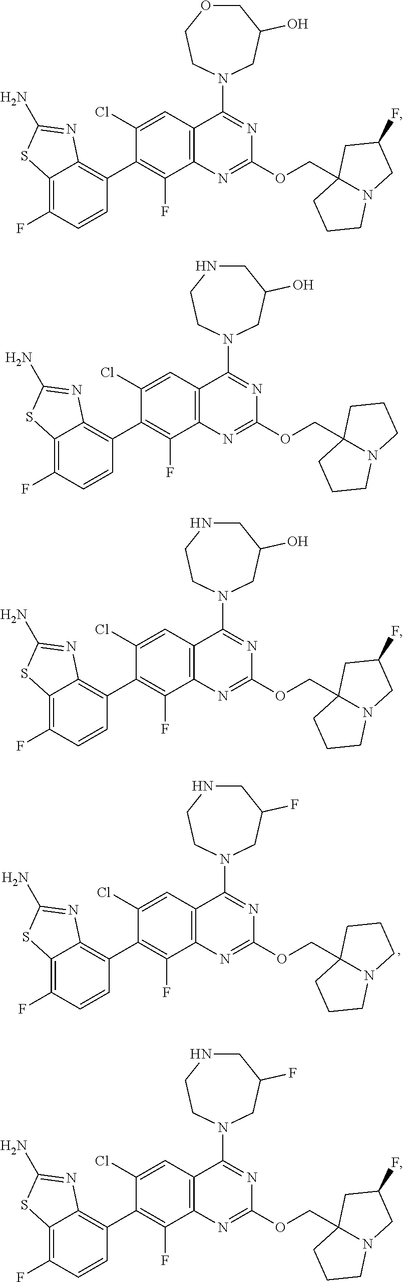 [SMILES: Nc1nc2c(-c3c(Cl)cc4c(N5CCOCC(O)C5)nc(OCC56CCCN5C[C@H](F)C6)nc4c3F)ccc(F)c2s1 ; Nc1nc2c(-c3c(Cl)cc4c(N5CCNCC(O)C5)nc(OCC56CCCN5CCC6)nc4c3F)ccc(F)c2s1 ; Nc1nc2c(-c3c(Cl)cc4c(N5CCNCC(O)C5)nc(OCC56CCCN5C[C@H](F)C6)nc4c3F)ccc(F)c2s1 ; Nc1nc2c(-c3c(Cl)cc4c(N5CCNCC(F)C5)nc(OCC56CCCN5CCC6)nc4c3F)ccc(F)c2s1 ; Nc1nc2c(-c3c(Cl)cc4c(N5CCNCC(F)C5)nc(OCC56CCCN5C[C@H](F)C6)nc4c3F)ccc(F)c2s1]

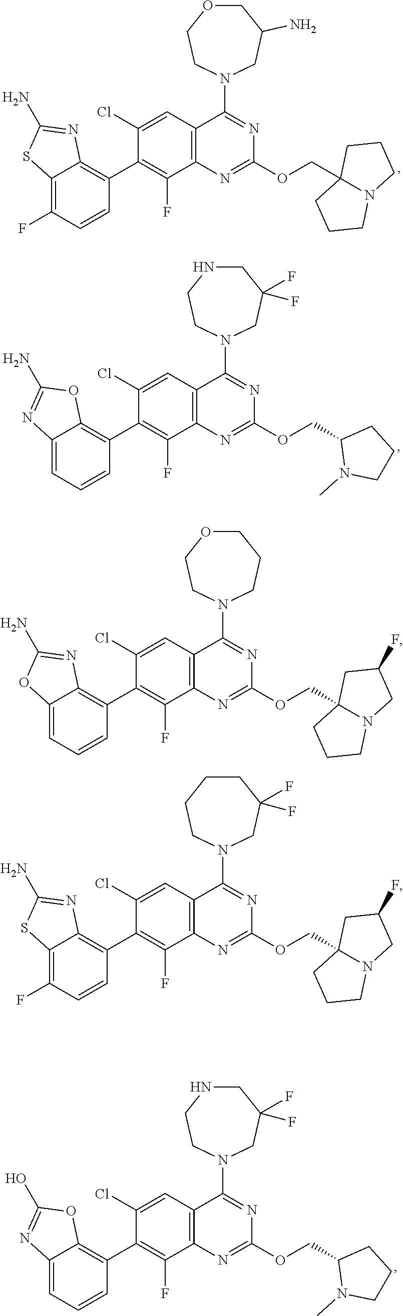 [SMILES: Nc1nc2c(-c3c(Cl)cc4c(N5CCOCC(N)C5)nc(OCC56CCCN5CCC6)nc4c3F)ccc(F)c2s1 ; CN1CCC[C@H]1COc1nc(N2CCNCC(F)(F)C2)c2cc(Cl)c(-c3cccc4nc(N)oc34)c(F)c2n1 ; Nc1nc2c(-c3c(Cl)cc4c(N5CCCOCC5)nc(OC[C@@]56CCCN5C[C@H](F)C6)nc4c3F)cccc2o1 ; Nc1nc2c(-c3c(Cl)cc4c(N5CCCCC(F)(F)C5)nc(OC[C@@]56CCCN5C[C@H](F)C6)nc4c3F)ccc(F)c2s1 ; CN1CCC[C@H]1COc1nc(N2CCNCC(F)(F)C2)c2cc(Cl)c(-c3cccc4nc(O)oc34)c(F)c2n1]

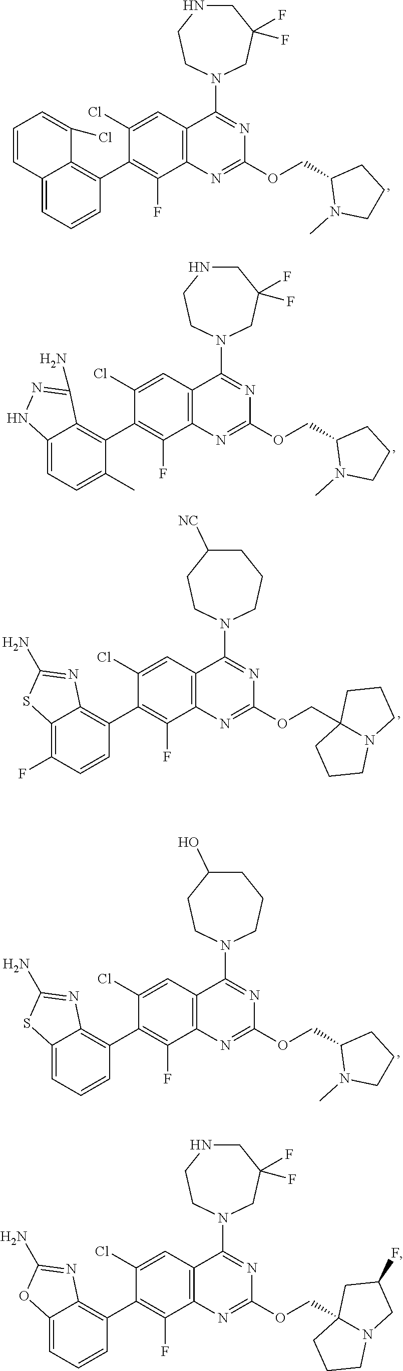 [SMILES: CN1CCC[C@H]1COc1nc(N2CCNCC(F)(F)C2)c2cc(Cl)c(-c3cccc4cccc(Cl)c34)c(F)c2n1 ; Cc1ccc2[nH]nc(N)c2c1-c1c(Cl)cc2c(N3CCNCC(F)(F)C3)nc(OC[C@@H]3CCCN3C)nc2c1F ; N#CC1CCCN(c2nc(OCC34CCCN3CCC4)nc3c(F)c(-c4ccc(F)c5sc(N)nc45)c(Cl)cc23)CC1 ; CN1CCC[C@H]1COc1nc(N2CCCC(O)CC2)c2cc(Cl)c(-c3cccc4sc(N)nc34)c(F)c2n1 ; Nc1nc2c(-c3c(Cl)cc4c(N5CCNCC(F)(F)C5)nc(OC[C@@]56CCCN5C[C@H](F)C6)nc4c3F)cccc2o1]

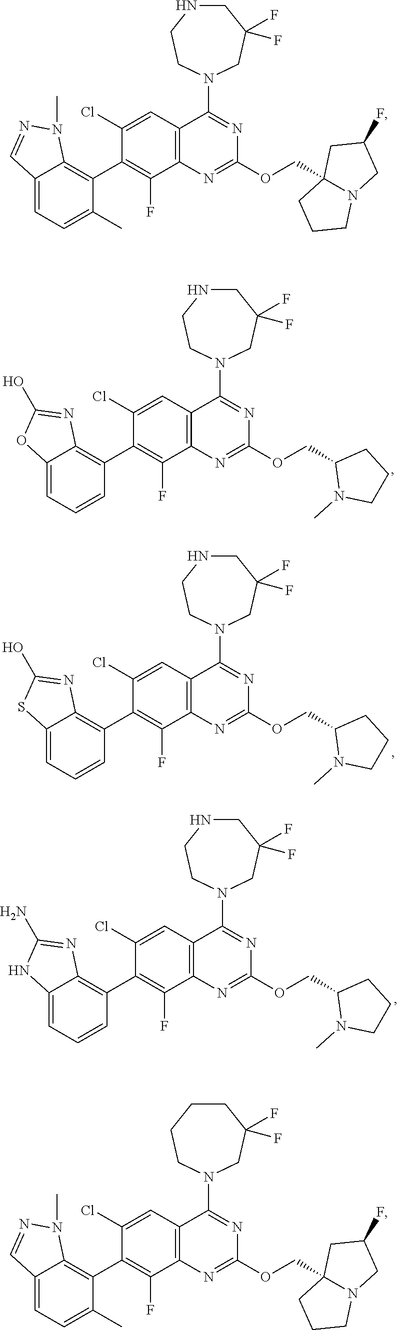 [SMILES: Cc1ccc2cnn(C)c2c1-c1c(Cl)cc2c(N3CCNCC(F)(F)C3)nc(OC[C@@]34CCCN3C[C@H](F)C4)nc2c1F ; CN1CCC[C@H]1COc1nc(N2CCNCC(F)(F)C2)c2cc(Cl)c(-c3cccc4oc(O)nc34)c(F)c2n1 ; CN1CCC[C@H]1COc1nc(N2CCNCC(F)(F)C2)c2cc(Cl)c(-c3cccc4sc(O)nc34)c(F)c2n1 ; CN1CCC[C@H]1COc1nc(N2CCNCC(F)(F)C2)c2cc(Cl)c(-c3cccc4[nH]c(N)nc34)c(F)c2n1 ; Cc1ccc2cnn(C)c2c1-c1c(Cl)cc2c(N3CCCCC(F)(F)C3)nc(OC[C@@]34CCCN3C[C@H](F)C4)nc2c1F]

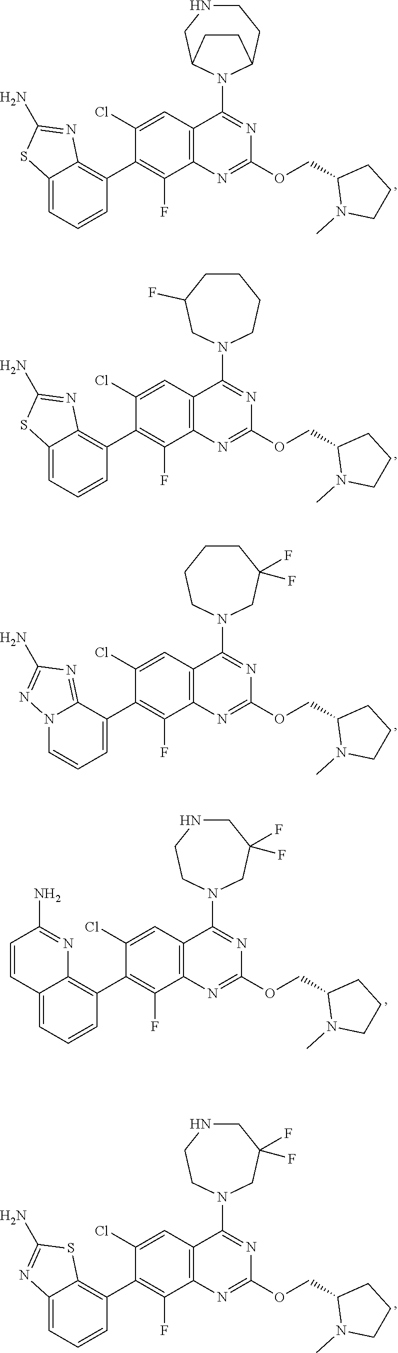 [SMILES: CN1CCC[C@H]1COc1nc(N2C3CCNCC2CC3)c2cc(Cl)c(-c3cccc4sc(N)nc34)c(F)c2n1 ; CN1CCC[C@H]1COc1nc(N2CCCCC(F)C2)c2cc(Cl)c(-c3cccc4sc(N)nc34)c(F)c2n1 ; CN1CCC[C@H]1COc1nc(N2CCCCC(F)(F)C2)c2cc(Cl)c(-c3cccn4nc(N)nc34)c(F)c2n1 ; CN1CCC[C@H]1COc1nc(N2CCNCC(F)(F)C2)c2cc(Cl)c(-c3cccc4ccc(N)nc34)c(F)c2n1 ; CN1CCC[C@H]1COc1nc(N2CCNCC(F)(F)C2)c2cc(Cl)c(-c3cccc4nc(N)sc34)c(F)c2n1]

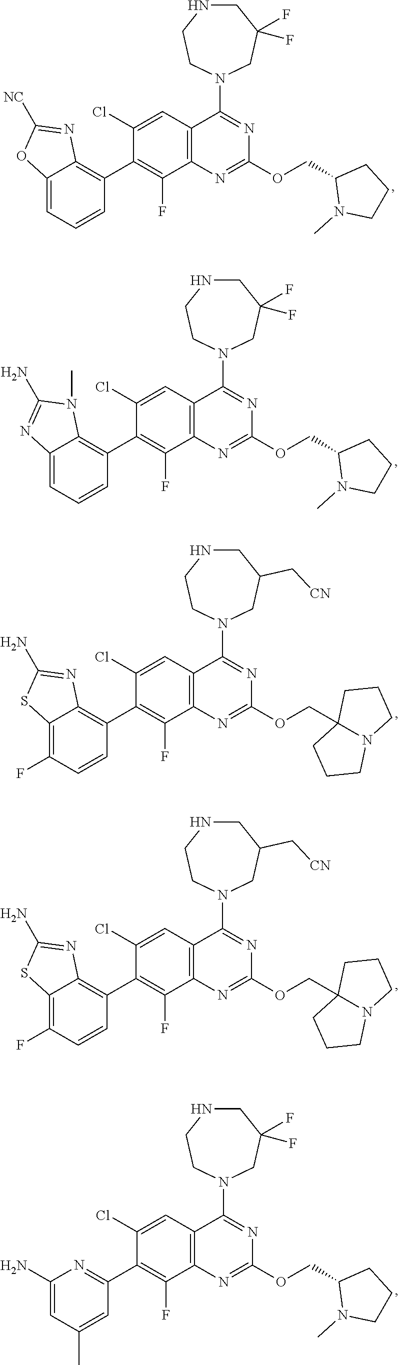 [SMILES: CN1CCC[C@H]1COc1nc(N2CCNCC(F)(F)C2)c2cc(Cl)c(-c3cccc4oc(C#N)nc34)c(F)c2n1 ; CN1CCC[C@H]1COc1nc(N2CCNCC(F)(F)C2)c2cc(Cl)c(-c3cccc4nc(N)n(C)c34)c(F)c2n1 ; N#CCC1CNCCN(c2nc(OCC34CCCN3CCC4)nc3c(F)c(-c4ccc(F)c5sc(N)nc45)c(Cl)cc23)C1 ; N#CCC1CNCCN(c2nc(OCC34CCCN3CCC4)nc3c(F)c(-c4ccc(F)c5sc(N)nc45)c(Cl)cc23)C1 ; Cc1cc(N)nc(-c2c(Cl)cc3c(N4CCNCC(F)(F)C4)nc(OC[C@@H]4CCCN4C)nc3c2F)c1]

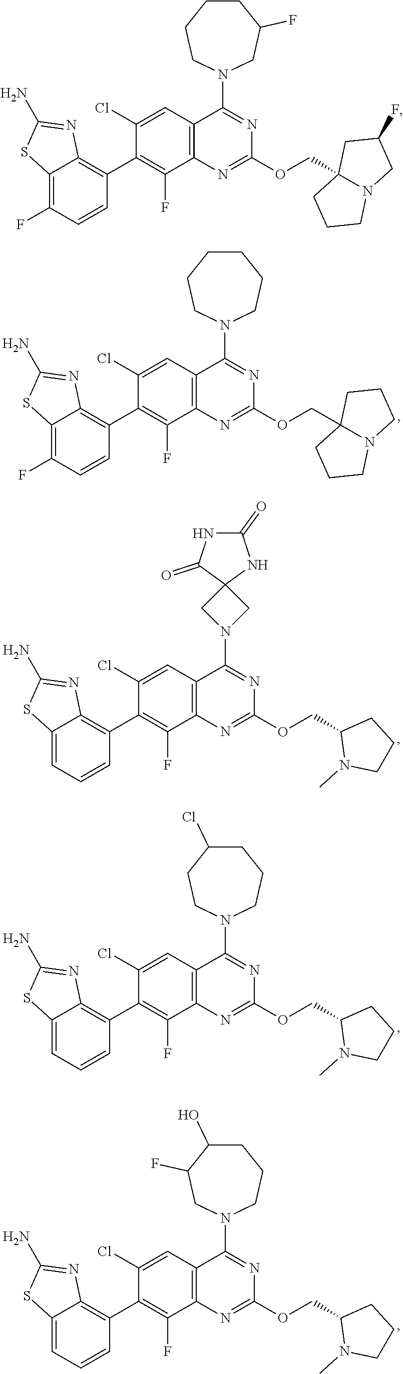 [SMILES: Nc1nc2c(-c3c(Cl)cc4c(N5CCCCC(F)C5)nc(OC[C@@]56CCCN5C[C@H](F)C6)nc4c3F)ccc(F)c2s1 ; Nc1nc2c(-c3c(Cl)cc4c(N5CCCCCC5)nc(OCC56CCCN5CCC6)nc4c3F)ccc(F)c2s1 ; CN1CCC[C@H]1COc1nc(N2CC3(C2)NC(=O)NC3=O)c2cc(Cl)c(-c3cccc4sc(N)nc34)c(F)c2n1 ; CN1CCC[C@H]1COc1nc(N2CCCC(Cl)CC2)c2cc(Cl)c(-c3cccc4sc(N)nc34)c(F)c2n1 ; CN1CCC[C@H]1COc1nc(N2CCCC(O)C(F)C2)c2cc(Cl)c(-c3cccc4sc(N)nc34)c(F)c2n1]

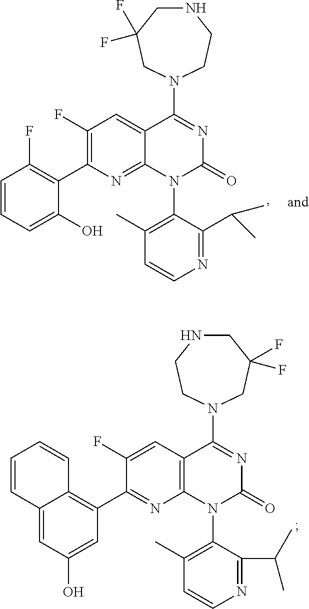 [SMILES: Cc1ccnc(C(C)C)c1-n1c(=O)nc(N2CCNCC(F)(F)C2)c2cc(F)c(-c3c(O)cccc3F)nc21 ; Cc1ccnc(C(C)C)c1-n1c(=O)nc(N2CCNCC(F)(F)C2)c2cc(F)c(-c3cc(O)cc4ccccc34)nc21]

or a pharmaceutically acceptable salt or solvate thereof.

The compounds of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, are Ras modulators (including Ras inhibitors) and have a wide range of applications in therapeutics, diagnostics, and other biomedical research.

In an aspect is provided a compound of Formula (XI-1), or a pharmaceutically acceptable salt or solvate thereof:

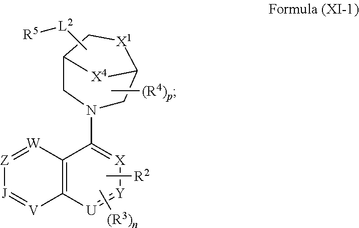

wherein:
X is C or N;
X¹ is selected from C(R⁴)(R⁶), C═N—OR⁴,)C═NN(R⁴)(R⁴), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
X⁴ is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X⁵C(R⁴)₂—, —C(R⁴)₂X³—, —C(H)C(R⁴)₂—, —C(R⁴)₂C(H)—, and —C(R⁴)₂C(R⁴)₂—;
X⁵ is selected from C(O), C═N—OR⁴, C═NNR⁴R⁶, N(R⁴), N(R⁶), S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N;
Z is N or C(R⁸), wherein at least one of U, X, and Z is not N;
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In an aspect is provided a compound of Formula (XI-2), or a pharmaceutically acceptable salt or solvate thereof:

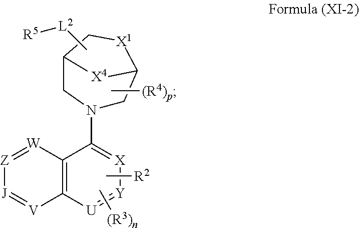

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
X⁴ is selected from X⁵, —CH₂—, —X⁵CH₂—, —CH₂X³—, —CH₂CH₂—, —C(H)(R⁴)—, —C(H)(R⁴)—, —X⁵C(H)(R⁴)—, —C(H)(R⁴)X³—, —C(H)(R⁴)CH₂—, —C(H)(R⁴)C(H)(R⁴)—, —C(R⁴)₂—, —X⁵—, —C(R⁴)₂—, —C(R⁴)₂X³—, —C(H)C(R⁴)₂—, —C(R⁴)₂C(H)—, and —C(R⁴)₂C(R⁴)₂—;
X⁵ is selected from C(O), C—N—OR⁴, C═NNR⁴R⁶, N(R⁴), N(R⁶), S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N;
Z is N or C(R⁸), wherein at least one of U, X, and Z is not N;
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In some embodiments is a compound of Formula (XI-1) or (XI-2) having the structure of Formula (XIa), or a pharmaceutically acceptable salt or solvate thereof:

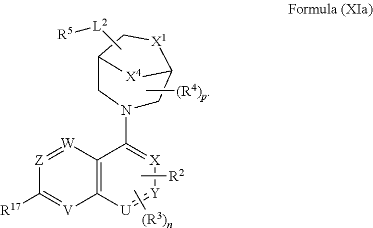

In some embodiments is a compound of Formula (XI-1) or (XI-2) or (XIa), or a pharmaceutically acceptable salt or solvate thereof, wherein Y is C. In some embodiments is a compound of Formula (XI-1) or (XI-2) or (XIa), or a pharmaceutically acceptable salt or solvate thereof, wherein Y is N. In some embodiments is a compound of Formula (XI-1) or (XI-2) or (XIa), or a pharmaceutically acceptable salt or solvate thereof, wherein Y is C(O).

In some embodiments is a compound of Formula (XI-1) or (XI-2) or (XIa), or a pharmaceutically acceptable salt or solvate thereof, wherein X is C. In some embodiments is a compound of Formula (XI-1) or (XI-2) or (XIa), or a pharmaceutically acceptable salt or solvate thereof, wherein X is N.

In some embodiments is a compound of Formula (XI-1) or (XI-2) or (XIa), or a pharmaceutically acceptable salt or solvate thereof, wherein U is C. In some embodiments is a compound of Formula (XI-1) or (XI-2) or (XIa), or a pharmaceutically acceptable salt or solvate thereof, wherein U is N. In some embodiments is a compound of Formula (XI-1) or (XI-2) or (XIa), or a pharmaceutically acceptable salt or solvate thereof, wherein U is C(O).

In some embodiments is a compound of Formula (XI-1) or (XI-2) having the structure of Formula (XIb), or a pharmaceutically acceptable salt or solvate thereof:

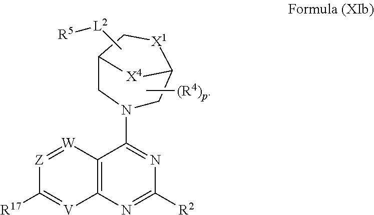

In some embodiments is a compound of Formula (XI-1) or (XI-2) having the structure of Formula (XIc), or a pharmaceutically acceptable salt or solvate thereof:

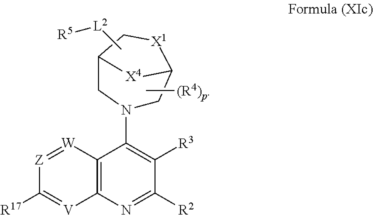

In some embodiments is a compound of Formula (XI-1) or (XI-2) having the structure of Formula (XId), or a pharmaceutically acceptable salt or solvate thereof:

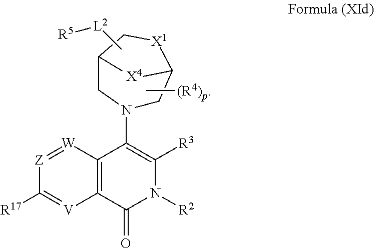

In some embodiments is a compound of Formula (XI-1) or (XI-2) having the structure of Formula (XIe), or a pharmaceutically acceptable salt or solvate thereof:

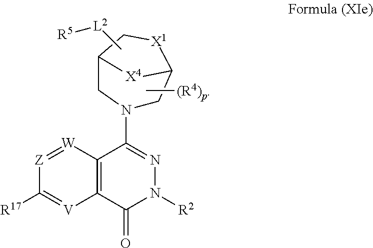

In some embodiments is a compound of Formula (XI-1) or (XI-2) having the structure of Formula (XIf), or a pharmaceutically acceptable salt or solvate thereof:

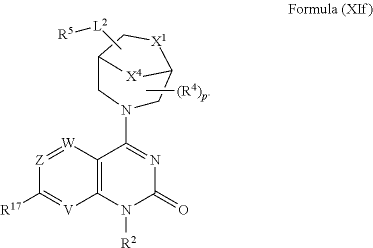

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is hydrogen, halogen, —CN, C₁₋₆alkyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is hydrogen, halogen, C₁₋₆alkyl, —OR¹², —SR¹², —N(H)(R¹²), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is hydrogen, halogen, C₁₋₆alkyl, or —OR¹², wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is hydrogen or halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is hydrogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is chloro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is C(R⁸) and R⁸ is fluoro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein Z is N.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is hydrogen, halogen, —CN, C₁₋₆alkyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is hydrogen, halogen, C₁₋₆alkyl, —OR¹², —SR¹², —N(R¹²)(R¹³), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is hydrogen, halogen, C₁₋₆alkyl, or —OR¹², wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is hydrogen or halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is hydrogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is chloro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is C(R¹⁶) and R¹⁶ is fluoro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein V is N.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is hydrogen, halogen, —CN, C₁₋₆alkyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is hydrogen, halogen, C₁₋₆alkyl, —OR¹², —SR¹², —N(R¹²)(R¹³), —C(O)OR¹², —C(O)R¹⁵, —C(O)N(R¹²)(R¹³), —S(O)₂R¹⁵, or —S(O)₂N(R¹²)(R¹³)—, wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is hydrogen, halogen, C₁₋₆alkyl, or —OR¹², wherein C₁₋₆alkyl is optionally substituted with one, two, or three R^{20c}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is hydrogen or halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is hydrogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is halogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is chloro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is C(R¹⁸) and R¹⁸ is fluoro. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein W is N.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is N(R⁴). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is N(H). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is N(R⁶). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is O. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is S. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is S(O)₂. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is C(R⁴)(R⁶). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is C(H)(R⁴). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is CH₂. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X¹ is C(H)(R⁶).

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X⁴ is selected from X⁵ and —CH₂—. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X⁴ is X⁵. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein X⁵ is N(R¹). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹ is selected from hydrogen and C₁₋₆alkyl. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹ is hydrogen. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹ is C₁₋₆alkyl.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R² is —OR¹².

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹² is C₁₋₆alkyl optionally substituted with one, two, or three R^{20d}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹² is C₂₋₉heterocycloalkyl optionally substituted with one, two, or three R^{20d}. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹² is —CH₂—C₂₋₉heterocycloalkyl optionally substituted with one, two, or three R^{20d}.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R² is selected from

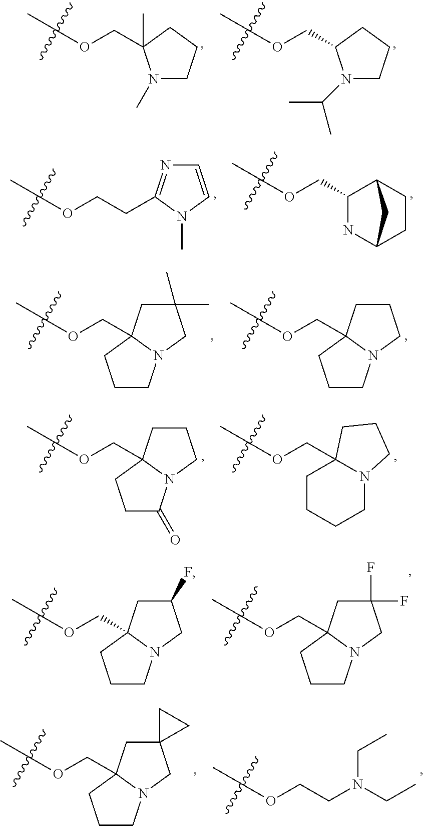

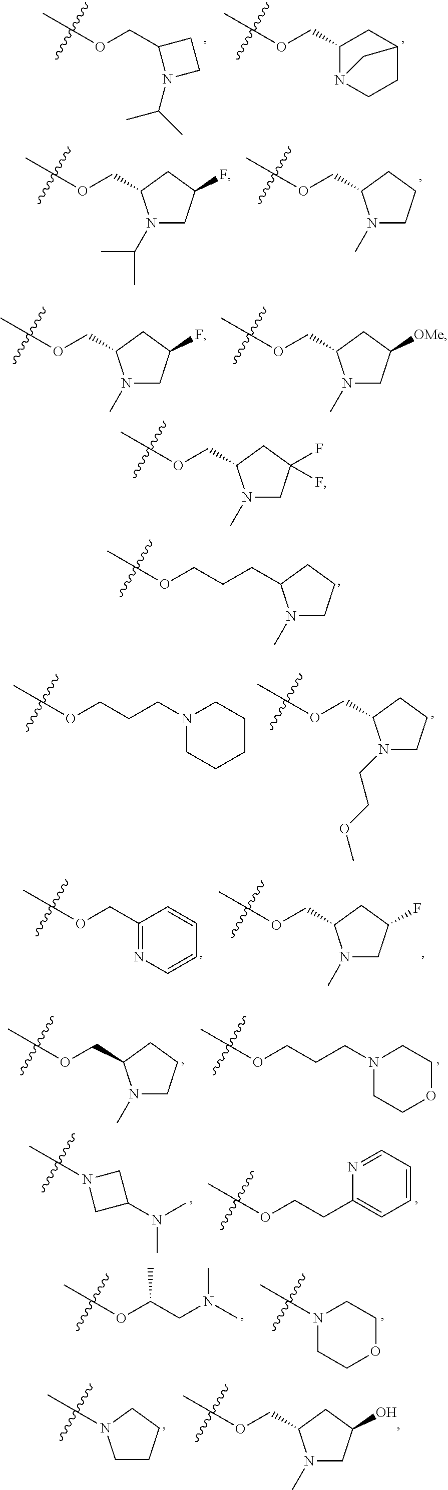

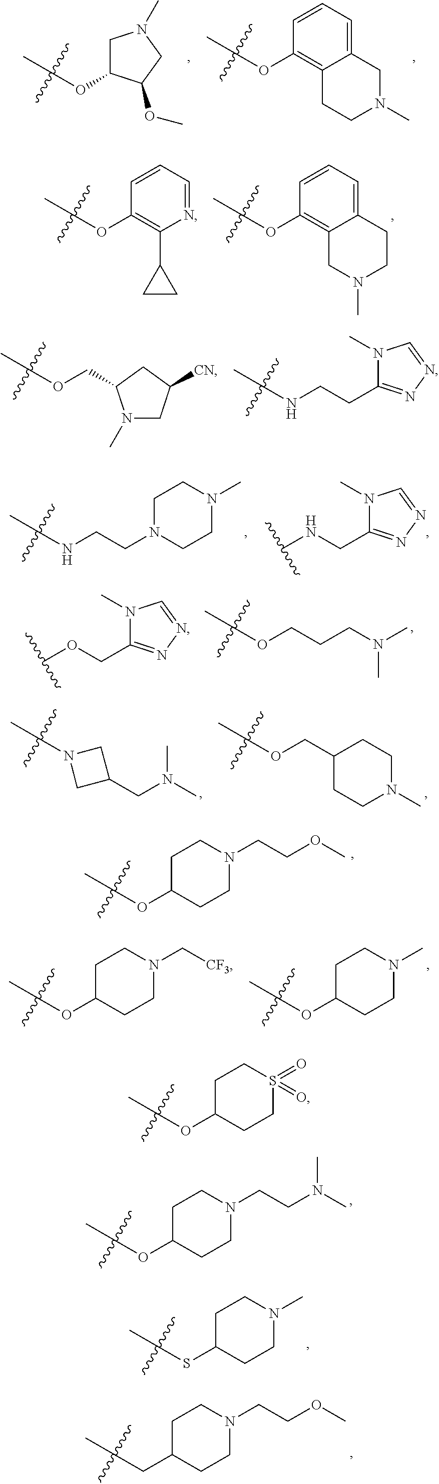

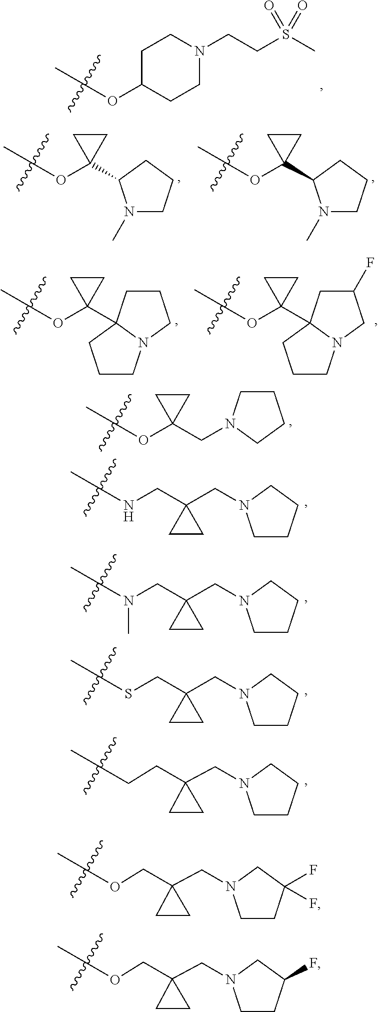

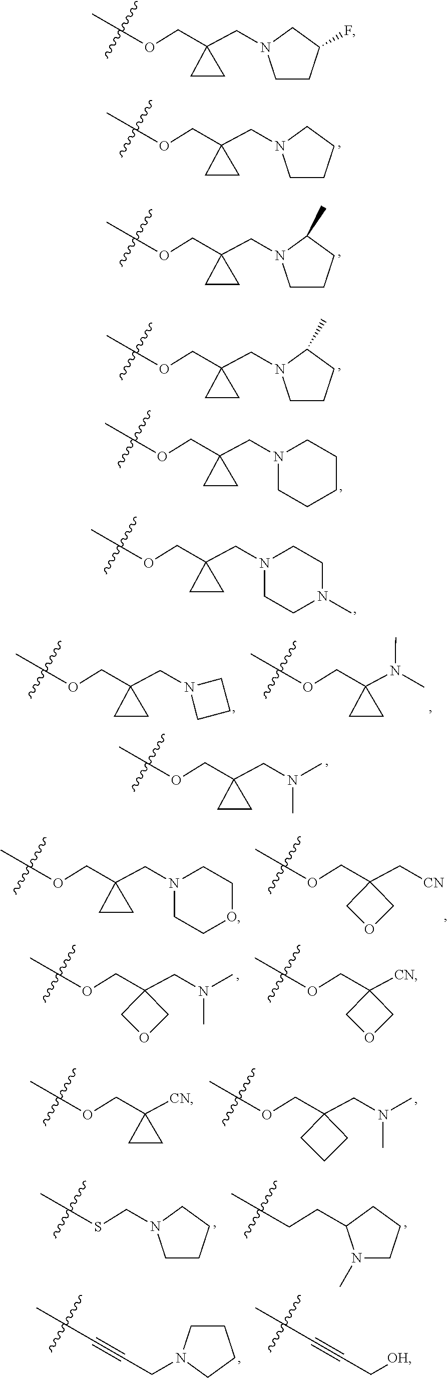

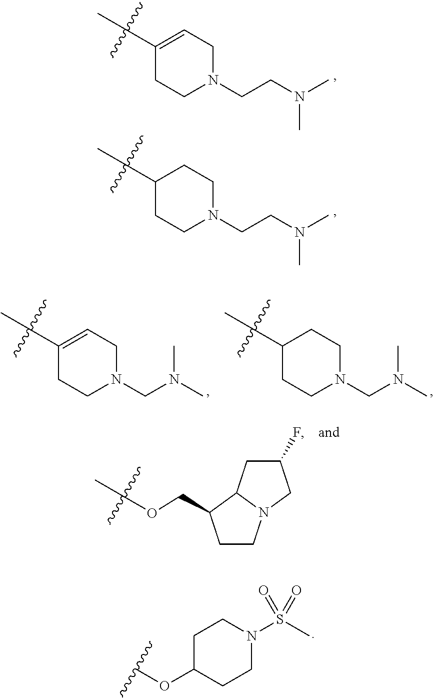

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein L¹ is a bond. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein L¹ is O.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹⁹ is C₁₋₉heteroaryl optionally substituted with one, two, or three R²⁰ⁱ. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein R¹⁹ is C₆₋₁₀aryl optionally substituted with one, two, or three R²⁰ⁱ.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 1, 2, 3, 4, or 5. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 0, 1, 2, or 3. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 0. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 1. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 2. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein p is 3.

In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein each L² is selected from a bond, C₁-C₆alkyl, and —C(O)—. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein each L² is a bond. In some embodiments is a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein each L² is C₁-C₆alkyl.

In some embodiments is a compound of Formula (XI-1), (XIa), (XIb), (XIc), (XId), (XIe), (XIf), (XII-1), (XIIa), (XIIb), (XIIc), or (XIId), or a pharmaceutically acceptable salt or solvate thereof, wherein each R⁵ is hydrogen. In some embodiments is a compound of Formula (XI-1), (XIa), (XIb), (XIc), (XId), (XIe), (XIf), (XII-1), (XIIa), (XIIb), (XIIc), or (XIId), or a pharmaceutically acceptable salt or solvate thereof, wherein each R⁵ is a group other than an electrophilic moiety capable of forming a covalent bond with the cysteine residue at position 12 of a KRAS protein. In some embodiments is a compound of Formula (XI-1), (XIa), (XIb), (XIc), (XId), (XIe), (XIf), (XII-1), (XIIa), (XIIb), (XIIc), or (XIId), or a pharmaceutically acceptable salt or solvate thereof, wherein each R⁵ is selected from: —H, —NH₂, —OH, —NH(C) (alkyl).

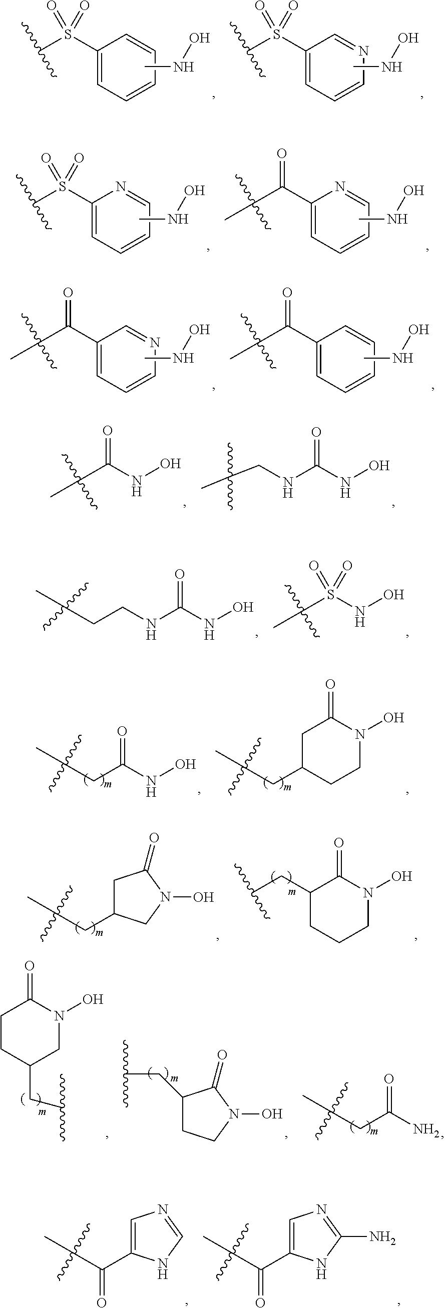

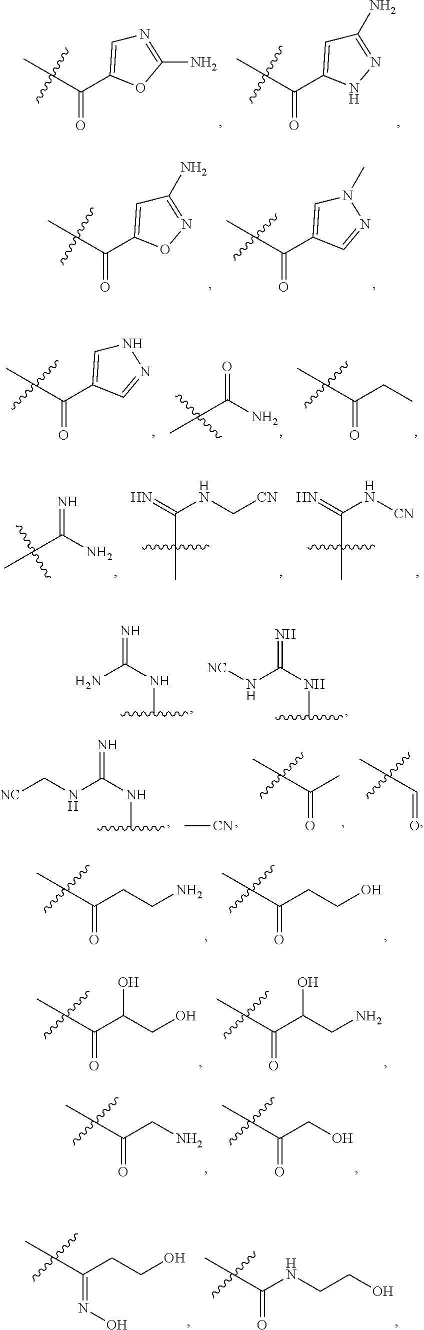

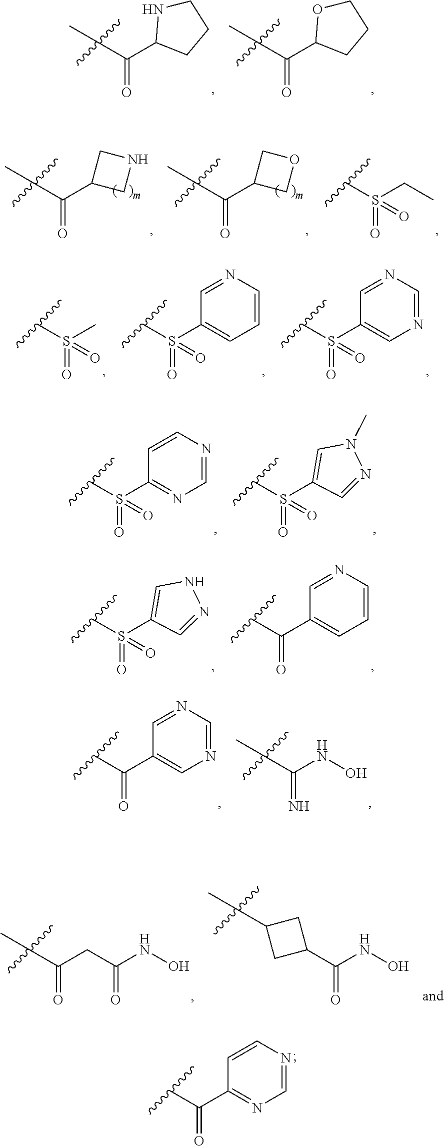

and m, when present, is 0, 1, 2, or 3.

In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently halogen. In further embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —CN. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —OH. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —NH₂. In further embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —C(O)OH. In select embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —OC(O)NH₂. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —NHC(O)NH₂. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —NHC(O)OH. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —NHS(O)₂H. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —C(O)NH₂. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently each R⁵ is independently hydrogen. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently oxo. In further embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently C₁₋₆alkyl optionally substituted with one, two, or three R^{20a}. In select embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently C₃₋₆cycloalkyl optionally substituted with one, two, or three R^{20a}. In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently C₂₋₉heterocycloalkyl optionally substituted with one, two, or three R^{20a}. In some embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently C₆₋₁₀aryl optionally substituted with one, two, or three R^{20a}. In further embodiments of the subject compound of Formula) (XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently each R⁵ is independently C₁₋₉heteroaryl optionally substituted with one, two, or three R^{20a}. In select embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —OR¹². In embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —N(R¹²)(R¹³). In select embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently N(R¹⁴)S(O)₂R¹⁵. In select embodiments of the subject compound of Formula)(XI-1), (XIa), (XIb), (XIc), (XId), (XIe), (XIf), (XII-1), (XIIa), (XIIb), (XIIc), or (XIId), or a pharmaceutically acceptable salt or solvate thereof, each R⁵ is independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (XI-2), (XIa), (XIb), (XIc), (XId), (XIe), (XIf), (XII-2), (XIIa), (XIIb), (XIIc), or (XIId), or a pharmaceutically acceptable salt or solvate thereof, wherein each R⁵ is an electrophilic moiety capable of forming a covalent bond with a residue at position 12 of a KRAS protein, e.g., the cysteine residue at position 12. In some embodiments is a compound of Formula (XI-2), (XIa), (XIb), (XIc), (XId), (XIe), (XIf), (XII-2), (XIIa), (XIIb), (XIIc), or (XIId), or a pharmaceutically acceptable salt or solvate thereof, wherein each R⁵ is an electrophilic moiety capable of forming a covalent bond with the cysteine residue at position 12 of a KRAS protein. In some embodiments is a compound of Formula (XI-2), (XIa), (XIb), (XIc), (XId), (XIe), (XIf), (XII-2), (XIIa), (XIIb), (XIIc), or (XIId), or a pharmaceutically acceptable salt or solvate thereof, wherein each R⁵ is selected from the group consisting of

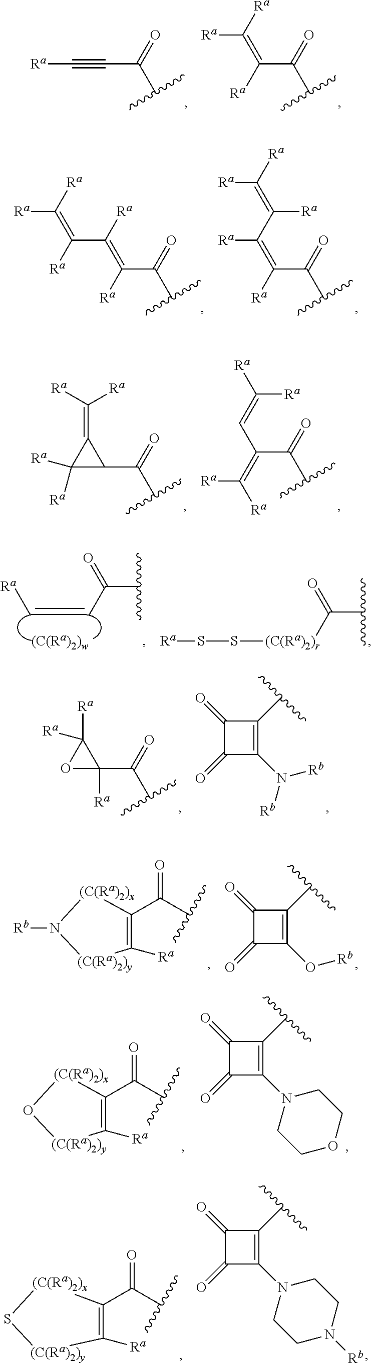

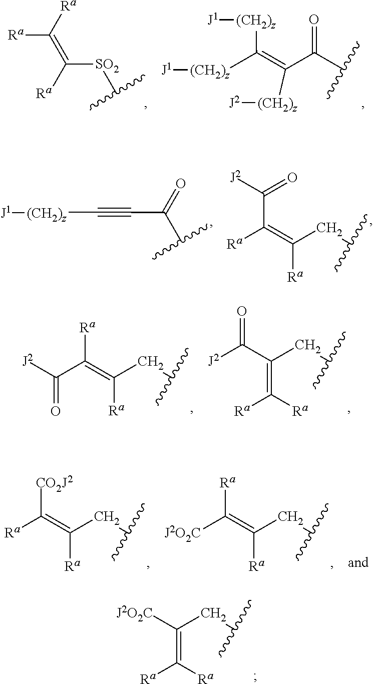

where each R^{a} is independently hydrogen, C₁₋₆alkyl, carboxy, C₁₋₆carboalkoxy, phenyl, C₂₋₇carboalkyl, R^{c}—(C(R^{b})₂)_{z}, R^{c}—(C(R^{b})₂)_{w}-M-(C(R^{b})₂), (R^{d})(R^{c})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{e} is —NR^{b}R^{b} or —OR^{b}; R^{d} and R^{e} are each, independently, —(C(R^{b})₂)ᵣ—NR^{b}R^{b}, or —(C(R^{b})₂)—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, each R⁵ is selected from the group consisting of:

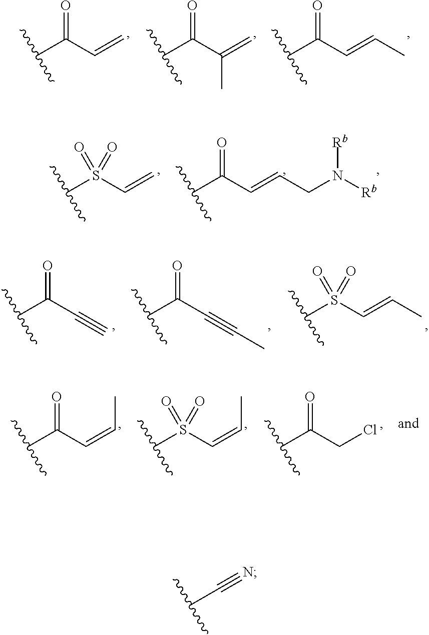

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In an aspect is provided a compound of Formula (XII-1), or a pharmaceutically acceptable salt or solvate thereof:

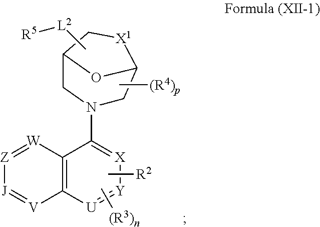

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴); N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N, wherein two of U, Y, and X are N;
Z is N or C(R⁸);
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—; each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, naphthalenyl, and C₁₋₉heteroaryl, wherein C₃₋₆cycloalkyl, C₂₋₉heterocycloalkyl, naphthalenyl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently 8selected from halogen, —CN, C₁₋₆alkyl, C₂₋₉alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In an aspect is provided a compound of Formula (XII-2), or a pharmaceutically acceptable salt or solvate thereof:

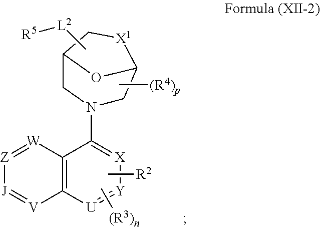

wherein:

X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴); N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N, wherein two of U, Y, and X are N;
Z is N or C(R⁸);
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—; each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, naphthalenyl, and C₁₋₉heteroaryl, wherein C₃₋₆cycloalkyl, C₂₋₉heterocycloalkyl, naphthalenyl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently 8selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In some embodiments is a compound of Formula (XII-1) or (XII-2) having the structure of Formula (XIIa), or a pharmaceutically acceptable salt or solvate thereof:

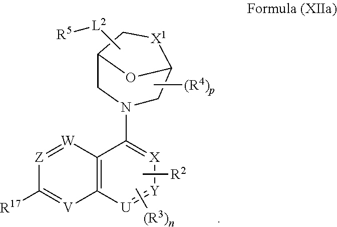

In some embodiments is a compound of Formula (XII-1) or (XII-2) or (XIIa), or a pharmaceutically acceptable salt or solvate thereof, wherein Y is C. In some embodiments is a compound of Formula (XII-1) or (XII-2) or (XIIa), or a pharmaceutically acceptable salt or solvate thereof, wherein Y is N. In some embodiments is a compound of Formula (XII-1) or (XII-2) or (XIIa), or a pharmaceutically acceptable salt or solvate thereof, wherein Y is C(O).

In some embodiments is a compound of Formula (XII-1) or (XII-2) or (XIIa), or a pharmaceutically acceptable salt or solvate thereof, wherein X is C. In some embodiments is a compound of Formula (XII-1) or (XII-2) or (XIIa), or a pharmaceutically acceptable salt or solvate thereof, wherein X is N.

In some embodiments is a compound of Formula (XII-1) or (XII-2) or (XIIa), or a pharmaceutically acceptable salt or solvate thereof, wherein U is C. In some embodiments is a compound of Formula (XII-1) or (XII-2) or (XIIa), or a pharmaceutically acceptable salt or solvate thereof, wherein U is N. In some embodiments is a compound of Formula (XII-1) or (XII-2) or (XIIa), or a pharmaceutically acceptable salt or solvate thereof, wherein U is C(O).

In some embodiments is a compound of Formula (XII-1) or (XII-2) having the structure of Formula (XIIb), or a pharmaceutically acceptable salt or solvate thereof:

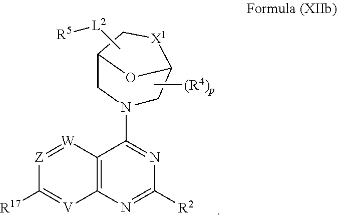

In some embodiments is a compound of Formula (XII-1) or (XII-2) having the structure of Formula (XIIc), or a pharmaceutically acceptable salt or solvate thereof:

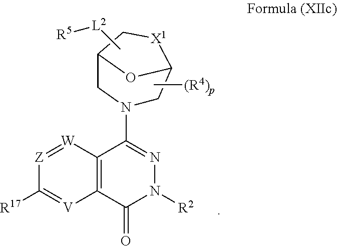

In some embodiments is a compound of Formula (XII-1) or (XII-2) having the structure of Formula (XIId), or a pharmaceutically acceptable salt or solvate thereof:

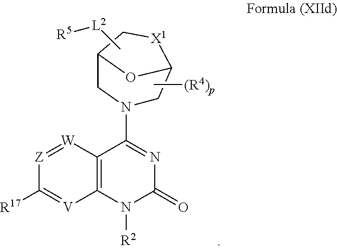

The compounds of Formula (XXI-1), (XXI-2), (XXIa), (XXIb), (XXIc), (XXII), (XXIIa), (XXIIb), (XXIIc), (XXIId), (XXIle), (XXIIf), (XXIIg), (XXIII-1), (XXIII-2), (XXIV-1), or (XXIV-2) or a pharmaceutically acceptable salt or solvate thereof, are Ras modulators (including Ras inhibitors) and have a wide range of applications in therapeutics, diagnostics, and other biomedical research.

In an aspect is provided a compound of Formula (XXI-1), or a pharmaceutically acceptable salt or solvate thereof:

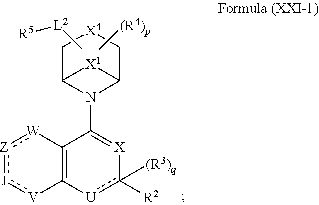

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R¹⁸)O, C(R^{1f})(R¹⁸)N(R^{1c}), and C(R^{1f})(R¹⁸);
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(—NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d},
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R¹²⁰;
L³ is selected from C₁-C₆alkyl, C₂₋₆alkenyl, C₂₋₆alkynyl, —O—, —N(R¹⁴)—, and —S—;
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXI-2), or a pharmaceutically acceptable salt or solvate thereof:

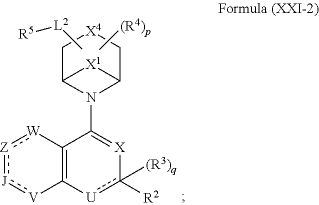

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁴)C═N—OR⁴, CH₂C═NN(R⁴)(R⁶), C(R⁴)(R⁶)C(O)N(R⁴), C(R⁴)(R⁴)N(R⁴), C(R⁴)(R⁴)N(R⁶), C(R⁴)(R⁴)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C—NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(—NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C—NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R¹⁵)(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₆heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d},
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12b};
L³ is selected from C₁-C₆alkyl, C₂₋₆alkenyl, C₂₋₆alkynyl, —O—, —N(R¹⁴)—, and —S—;
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments of the subject compound of Formula (XXI-1) or (XXI-2), or a pharmaceutically acceptable salt or solvate thereof, X is C(R³). In further embodiments of the subject compound of Formula (XXI-1) or (XXI-2), or a pharmaceutically acceptable salt or solvate thereof, X is N. In select embodiments of the subject compound of Formula (XXI-1) or (XXI-2), or a pharmaceutically acceptable salt or solvate thereof, X is CH.

In some embodiments of the subject compound of Formula (XXI-1) or (XXI-2), or a pharmaceutically acceptable salt or solvate thereof, U is C(R³). In further embodiments of the subject compound of Formula (XXI-1) or (XXI-2), or a pharmaceutically acceptable salt or solvate thereof, U is N(R³). In additional embodiments of the subject compound of Formula (XXI-1) or (XXI-2), or a pharmaceutically acceptable salt or solvate thereof, U is N. In embodiments of the subject compound of Formula (XXI-1) or (XXI-2), or a pharmaceutically acceptable salt or solvate thereof, U is C(O). In embodiments of the subject compound of Formula (XXI-1) or (XXI-2), or a pharmaceutically acceptable salt or solvate thereof, U is NH. In embodiments of the subject compound of Formula (XXI-1) or (XXI-2), or a pharmaceutically acceptable salt or solvate thereof, U is CH. In embodiments of the subject compound of Formula (XXI-1) or (XXI-2), or a pharmaceutically acceptable salt or solvate thereof, U is C(R³)₂. In embodiments of the subject compound of Formula (XXI-1) or (XXI-2), or a pharmaceutically acceptable salt or solvate thereof, U is S(O). In embodiments of the subject compound of Formula (XXI-1) or (XXI-2), or a pharmaceutically acceptable salt or solvate thereof, U is S(O)₂. In embodiments of the subject compound of Formula (XXI-1) or (XXI-2), or a pharmaceutically acceptable salt or solvate thereof, U is CH₂.

The subject compound of Formula (XXI-1) or (XXI-2), or a pharmaceutically acceptable salt or solvate thereof, may have the structure of Formula (XXIa):

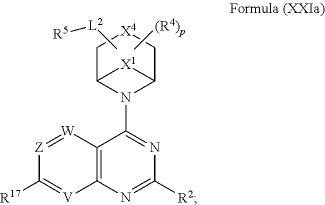

wherein X¹, X², R², L², R⁴, R⁵, p, R¹⁷, V, W, and Z are as described herein, including in embodiments.

The subject compound of Formula (XXI-1) or (XXI-2), or a pharmaceutically acceptable salt or solvate thereof, may have the structure of Formula (XXIb):

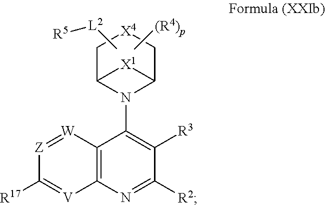

wherein X¹, X², R², R³, L², R⁴, R⁵, p, R¹⁷, V, W, and Z are as described herein, including in embodiments.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Z is C(R⁸). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is hydrogen or halogen. In additional embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Z is N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Z is N(R⁸). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Z is CH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Z is CH₂.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently chloro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently bromo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently iodo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁸ is independently C₁₋₆alkyl optionally substituted with one, two, or three R^{20c}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently chloro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently bromo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently iodo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{8a} is independently C₁₋₆alkyl optionally substituted with one, two, or three R^{20c}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, V is C(R¹⁶). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is hydrogen or halogen. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, V is N. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, V is CF.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR 12, —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently C₁₋₆alkyl optionally substituted with one, two, or three R^{20g}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently —OR^{12a}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently chloro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently bromo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁶ is independently iodo.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently C₁₋₆alkyl optionally substituted with one, two, or three R^{20g}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently —OR^{12a}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently chloro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently bromo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{16a} is independently iodo.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₁₋₆alkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₆alkenyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₆alkynyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₃₋₆cycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₉heterocycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₂₋₉heterocycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₆₋₁₀aryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₆₋₁₀aryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₁₋₉heteroaryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₁₋₉heteroaryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₁₋₆alkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₆alkenyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₆alkynyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₃₋₆cycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₂₋₉heterocycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₂₋₉heterocycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₆₋₁₀aryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₆₋₁₀aryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently —CH₂—C₁₋₉heteroaryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12a} is independently C₁₋₉heteroaryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is C(R¹⁸). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is N(R¹⁸). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, wherein R¹⁸ is hydrogen. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is N. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is N, C(R¹⁸)(R^{18a}), or C(R¹⁸). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is NH. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, W is CH.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently hydrogen.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{18a} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{18a} is independently hydrogen.

The subject compound of Formula (XXI-1) or (XXI-2), or a pharmaceutically acceptable salt or solvate thereof, may have the structure of Formula (XXIc):

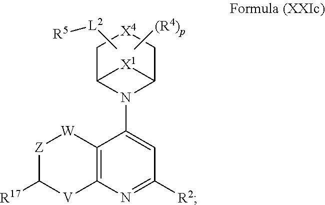

wherein X¹, X², R², L², R⁴, R⁵, p, R¹⁷, V, W, and Z are as described herein, including in embodiments.

In some embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, Z is C(R⁸)(R^{8a}). In further embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, R⁸ and R^{8a} are hydrogen. In further embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, Z is N(R⁸).

In some embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, V is C(R¹⁶)(R^{16a}). In embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, R¹⁶ and R^{16a} are hydrogen. In further embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, V is N(R¹⁶). In embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, V is NH.

In some embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, W is C(R¹⁸)(R^{18a}). In further embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, R¹⁸ and R^{18a} are hydrogen. In select embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, W is N(R¹⁸). In embodiments of the subject compound of Formula (XXI-1) or (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof, W is NH.

In an aspect is provided a compound of Formula (XXII-1), or a pharmaceutically acceptable salt or solvate thereof:

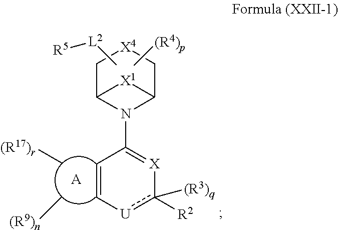

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴); S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C—N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(—O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl; C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;
R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXII-2), or a pharmaceutically acceptable salt or solvate thereof:

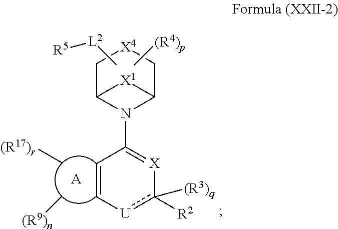

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S,)C(R⁴)(R⁴)SC(R⁴)(R⁶), C(R⁴)(R⁶)S(O),) C(R⁴)(R⁴)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴),) C(R⁴)(R⁶)N(R⁴)C(O)N(R⁴), C(R⁴)(R⁶)S(O)₂, C═NN(R⁴)(R⁴)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C—N—OR⁴, C—NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₆heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R¹⁸)O, C(R^{1f})(R¹⁸)N(R^{1c}), and C(R^{1f})(R¹⁸);
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl; and
[symbol] indicates a single or double bond such that all valences are satisfied.

In some embodiments of the subject compound of Formula (XXII-1) or (XXII-2), or a pharmaceutically acceptable salt or solvate thereof, Ring A is a 7-membered cycloalkyl ring. In some embodiments of the subject compound of Formula (XXII-1) or (XXII-2) or a pharmaceutically acceptable salt or solvate thereof, Ring A is a 7-membered heterocycloalkyl ring. In some embodiments of the subject compound of Formula (XXII-1) or (XXII-2) or a pharmaceutically acceptable salt or solvate thereof, Ring A is a saturated 7-membered cycloalkyl ring. In some embodiments of the subject compound of Formula (XXII-1) or (XXII-2)r a pharmaceutically acceptable salt or solvate thereof, Ring A is a saturated 7-membered heterocycloalkyl ring.

In an aspect is provided a compound having the formula (XXIIa-1), or a pharmaceutically acceptable salt or solvate thereof:

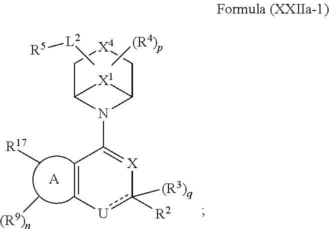

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴); S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶),) CH₂C(R⁴)(R⁶)CH₂, C(R⁴)(R⁶)C(R⁴)(R⁴)C(R⁴)(R⁶), C(R⁴)(R⁶)C—N—OR⁴, CH₂C═NN(R⁴)(R⁶), C(R⁴)(R⁴)C(O)N(R⁴),) C(R⁴)(R⁶)N(R⁴), C(R⁴)(R⁴)N(R⁶), C(R⁴)(R⁶)O,)C(R⁴)(R⁴)OC(R⁴)(R⁶), C(R⁴)(R⁴)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C—N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1e} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, or 5;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound having the formula (XXIIa-2), or a pharmaceutically acceptable salt or solvate thereof:

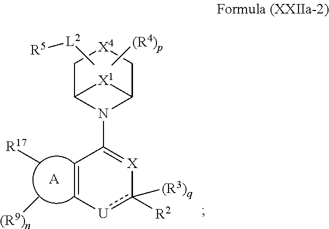

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁴)C(R⁴)(R⁶)C(R⁴)(R⁴), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, or 5;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments of the subject compound of Formula (XXII-1), (XXII-2), (XXIIa-1), or (XXIIa-2), or a pharmaceutically acceptable salt or solvate thereof, R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, or three R²⁰ⁱ.

In some embodiments of the subject compound of Formula (XXII-1), (XXII-2), (XXIIa-1), or (XXIIa-2), or a pharmaceutically acceptable salt or solvate thereof, R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, or three R²⁰ⁱ.

In an aspect is provided a compound of Formula (XXIIb-1), or a pharmaceutically acceptable salt or solvate thereof:

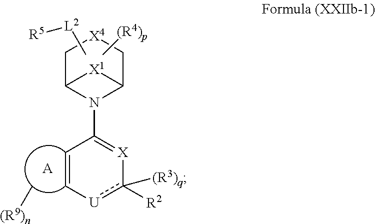

wherein:
Ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁴)N(R⁶), C(R⁴)(R⁶)O,)C(R⁴)(R⁶)OC(R⁴)(R⁴), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁴)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXIIb-2), or a pharmaceutically acceptable salt or solvate thereof:

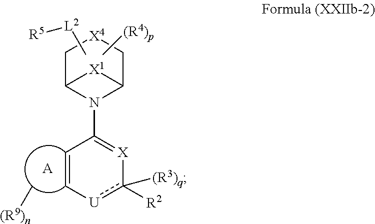

wherein:
Ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴),) C(R⁴)(R⁴)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C—NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b}.
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

The compound of Formula (XXII-1) or (XXII-2), or a pharmaceutically acceptable salt or solvate thereof, may have the structure of Formula (XXIIc):

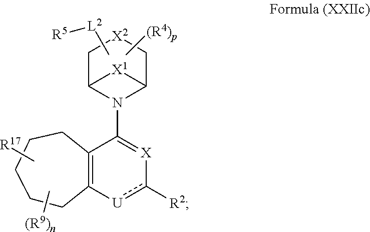

wherein U, X, X¹, X², R², L², R⁴, R⁵, p, R⁹, n, and R¹⁷ are as described herein, including in embodiments.

The compound of Formula (XXII-1) or (XXII-2) or (XXIIc), or a pharmaceutically acceptable salt or solvate thereof, may have the structure of Formula (XXIId):

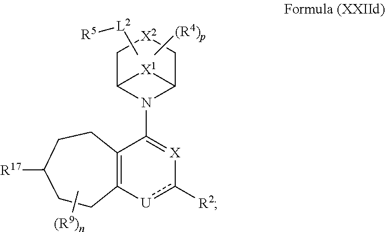

wherein U, X, X¹, X², R², L², R⁴, R⁵, p, R⁹, n, and R¹⁷ are as described herein, including in embodiments.

The compound of Formula (XXII-1) or (XXII-2) or (XXIIc), or a pharmaceutically acceptable salt or solvate thereof, may have the structure of Formula (XXIIe):

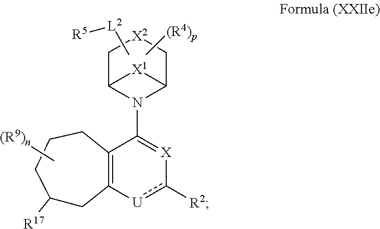

wherein U, X, X¹, X², R², L², R⁴, R⁵, p, Ron, and R¹⁷ are as described herein, including in embodiments.

The compound of Formula (XXII-1) or (XXII-2), or a pharmaceutically acceptable salt or solvate thereof, may have the structure of Formula (XXIIf):

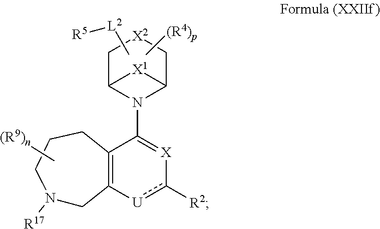

wherein U, X, X¹, X², R², L², R⁴, R⁵, p, R⁹,n, and R¹⁷ are as described herein, including in embodiments.

The compound of Formula (XXII-1) or (XXII-2), or a pharmaceutically acceptable salt or solvate thereof, may have the structure of Formula (XXIIg):

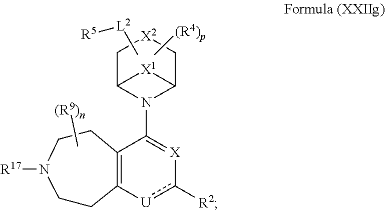

wherein U, X, X¹, X², R², L², R⁴, R⁵, p, R⁹, n, and R¹⁷ are as described herein, including in embodiments.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X is C(R³). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X is N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X is CH.

In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, U is C(R³). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, U is N(R³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, U is N. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, U is C(O).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently selected from halogen, C₁₋₆alkyl, C₃₋₆cycloalkyl, C₂₋₉heterocycloalkyl, C₆₋₁₀aryl, C₁₋₉heteroaryl, —OR¹², and —N(H)(R¹²), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20c}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently selected from halogen, C₁₋₆alkyl, —OR¹², and —N(H)(R¹²), wherein C₁₋₆alkyl is optionally substituted with one, two, or three R^{20c}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently a halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —CN. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₁₋₆alkyl optionally substituted with one, two, or three R^{20c}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₃₋₆cycloalkyl optionally substituted with one, two, or three R^{20c}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₂₋₉heterocycloalkyl optionally substituted with one, two, or three R^{20c}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₆₋₁₀aryl optionally substituted with one, two, or three R^{20c}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₁₋₉heteroaryl optionally substituted with one, two, or three R^{20c}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —OR¹². In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₁₋₆alkyl. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —OR¹². In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —N(H)(R¹²). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently F. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently C₁. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently Br. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently I. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —OH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —NH₂. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R⁹ is independently —CH₃.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, n is 1, 2, or 3. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, n is 0. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, n is 1. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, n is 2.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C—NN(R⁴)(R⁶) C(R⁴)(R⁶), C(O)N(R⁴)C(R⁴)(R⁶), S(O)₂N(R⁴)C(R⁴)(R⁶), S(O)N(R⁴)C(R⁴)(R⁶), and OC(O)N(R⁴)C(R⁴)(R⁶).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C═N—OR⁴, CH₂C═NN(R⁴)(R⁶), CH₂C(O)N(R⁴), CH₂N(R⁴), CH₂N(R⁶), CH₂O, CH₂S, CH₂S(O), CH₂S(═O)(═NR⁴), CH₂S(O)₂N(R⁴), CH₂N(R⁴)S(O)N(R⁴), CH₂N(R⁴)S(O)₂N(R⁴), CH₂S(O)N(R⁴), CH₂OC(O)N(R⁴), CH₂N(R⁴)C(O)N(R⁴), CH₂S(O)₂, C═NN(R⁴)(R⁶)CH₂, C(O)N(R⁴)CH₂, S(O)₂N(R⁴)CH₂, S(O)N(R⁴)CH₂, and OC(O)N(R⁴)CH₂.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂C(R⁴)(R⁶). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is —CH₂CH₂—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is C(R⁴)(R⁶). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is N(R⁴). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is N(R⁶). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is O. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is S. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂C(R⁴)(R⁶). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂N(R⁴). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂N(R⁶). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂O. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂S. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂C(OH)(R⁶). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂C(C₁-C₃ alkyl-CN)(R⁶). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂C(CH₂—CN)(R⁶). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂CH(C₁-C₃ alkyl-CN). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹ is CH₂CH(CH₂—CN).

In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is N(R¹). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is N(H). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is O. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is C(R⁴)₂. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is —CH₂—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is N(R⁶). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is S. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is S(O). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is —C(H)(R⁶)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is C(C₁-C₃ alkyl-CN)(R⁴). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is C(CH₂—CN)(R⁴). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is CH(C₁-C₃ alkyl-CN). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is CH(CH₂—CN). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is C(C₁-C₃ alkyl-CN)(R⁶). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁴ is C(CH₂—CN)(R⁶).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises only one R⁶. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises exactly two optionally different R⁶. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises exactly three optionally different R⁶.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises only one R⁵. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises exactly two optionally different R⁵. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, the compound of Formula (XXI-1), (XXI-2), (XXIa), (XXIb), (XXIc), (XXII), (XXIIa), (XXIIb), (XXIIc), (XXIId), (XXIIe), (XXIIf), (XXIIg), (XXIII), or (XXIV) comprises exactly three optionally different R⁵.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently hydrogen. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently halogen. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently oxo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —CN. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₁₋₆alkyl optionally substituted with one, two, or three R^{20a}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₃₋₆cycloalkyl optionally substituted with one, two, or three R^{20a}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₂₋₉heterocycloalkyl optionally substituted with one, two, or three R^{20a}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₆₋₁₀aryl optionally substituted with one, two, or three R^{20a}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₁₋₉heteroaryl optionally substituted with one, two, or three R^{20a}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —OR¹². In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —N(R¹²)(R¹³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)OR¹². In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)R¹⁵. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —OC(O)R¹⁵. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)N(R¹²)(R¹³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —N(R¹⁴)C(O)R¹⁵. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently C₁-C₃ alkyl-CN. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —O(C₁-C₃ alkyl-CN). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —NH(C₁-C₃ alkyl-CN). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)O(C₁-C₃ alkyl-CN). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)(C₁-C₃ alkyl-CN). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —OC(O)(C₁-C₃ alkyl-CN). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)NH(C₁-C₃ alkyl-CN). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —NHC(O)(C₁-C₃ alkyl-CN). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently CH₂—CN. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —O(CH₂—CN). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —NH(CH₂—CN). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)O(CH₂—CN). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)(CH₂—CN). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —OC(O)(CH₂—CN). In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —C(O)NH(CH₂—CN). In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R⁴ is independently —NHC(O)(CH₂—CN).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 0. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 1. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 2. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 3. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 4. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 5.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently a bond. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently C₁-C₆alkyl. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —O—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —C(O)—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)C(O)—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —C(O)N(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)₂—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)₂N(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)N(R¹⁴)—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)S(O)—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)S(O)₂—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —OCON(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)C(O)O—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —N(R¹⁴)C(O)N(R¹⁴)—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NH—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NHC(O)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —C(O)NH—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)₂NH—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —S(O)NH—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NHS(O)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NHS(O)₂—. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —OCONH—. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NHC(O)O—. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L² is independently —NHC(O)NH—.

In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1) or a pharmaceutically acceptable salt or solvate thereof, each R⁵ is independently hydrogen. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1) or a pharmaceutically acceptable salt or solvate thereof, each R⁵ is not hydrogen and is not an electrophilic moiety capable of forming a covalent bond with the cysteine residue at position 12 of a KRAS protein. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1) or a pharmaceutically acceptable salt or solvate thereof, each R⁵ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1) or a pharmaceutically acceptable salt or solvate thereof, each R⁵ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, and —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently halogen. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —CN. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OH. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NH₂. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)OH. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)NH₂. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)NH₂. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)OH. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHS(O)₂H. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)H. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)H. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)NH₂. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, R⁵ is independently hydrogen. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently halogen. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently oxo. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —CN. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₁₋₆alkyl optionally substituted with one, two, or three R^{20a}. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₃₋₆cycloalkyl optionally substituted with one, two, or three R^{20a}. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₂₋₉heterocycloalkyl optionally substituted with one, two, or three R^{20a}. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₆₋₁₀aryl optionally substituted with one, two, or three R^{20a}. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₁₋₉heteroaryl optionally substituted with one, two, or three R^{20a}.

In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OR¹². In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —N(R¹²)(R¹³). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)OR¹². In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)R¹⁵. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)R¹⁵. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)N(R¹²)(R¹³). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —N(R¹⁵)C(O)R¹⁵. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₁-C₃ alkyl-CN. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —O(C₁-C₃ alkyl-CN). In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NH(C₁-C₃ alkyl-CN). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)O(C₁-C₃ alkyl-CN). In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)(C₁-C₃ alkyl-CN). In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)(C₁-C₃ alkyl-CN). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)NH(C₁-C₃ alkyl-CN). In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)(C₁-C₃ alkyl-CN). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently CH₂—CN. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —O(CH₂—CN). In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NH(CH₂—CN). In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)O(CH₂—CN). In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)(CH₂—CN). In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)(CH₂—CN). In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)NH(CH₂—CN). In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)(CH₂—CN).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is hydrogen. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₁₋₆alkyl optionally substituted with one, two, or three R^{20a}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₁₋₆haloalkyl optionally substituted with one, two, or three R^{20a}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₃₋₆cycloalkyl optionally substituted with one, two, or three R^{20a}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is —CH₂—C₃₋₆cycloalkyl optionally substituted with one, two, or three R^{20a}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₂₋₉heterocycloalkyl optionally substituted with one, two, or three R^{20a}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is —CH₂—C₂₋₉heterocycloalkyl optionally substituted with one, two, or three R^{20a}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₆₋₁₀aryl optionally substituted with one, two, or three R^{20a}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is —CH₂—C₆₋₁₀aryl optionally substituted with one, two, or three R^{20a}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is —CH₂—C₁₋₉heteroaryl optionally substituted with one, two, or three R^{20a}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ is C₁₋₉heteroaryl optionally substituted with one, two, or three R^{20a}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently C₁₋₆alkyl optionally substituted with one, two, or three R^{20b}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently —OR¹². In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently —N(R¹²)(R¹³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently —OH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently —NH₂. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently chloro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently bromo. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R³ is independently iodo.

In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, q is 0. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, q is 1.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is C₁-C₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is —O—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is —N(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is —S—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is —NH—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L³ is selected from —O—, —N(R¹⁴)—, and —S—.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is C₁₋₆alkyl optionally substituted with one, two, or three R^{20d}. In select embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is C₂₋₉heterocycloalkyl optionally substituted with one, two, or three R^{20d}. In embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂—C₂₋₉heterocycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently C₁₋₆alkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently C₃₋₆cycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₃₋₆cycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently C₂₋₉heterocycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₂₋₉heterocycloalkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently C₆₋₁₀aryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₆₋₁₀aryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₁₋₉heteroaryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently C₁₋₉heteroaryl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₂₋₉heterocycloalkyl optionally substituted with one, two, or three halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently —CH₂—C₂₋₉heterocycloalkyl optionally substituted with one, two, or three fluoro. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is independently selected from —CH₂-hexahydro-IH-pyrrolizinyl, —CH₂-hexahydro-3H-pyrrolizin-3-one, —CH₂-hexahydro-1H-pyrrolo[2,1-c][1,4]oxazinyl, —CH₂-octahydroindolizinyl, —CH₂-hexahydropyrrolizine 4(IH)-oxide, —CH₂-azetidinyl, —CH₂-pyrrolidinyl, —CH₂-pyrrolidin-2-one, —CH₂-oxetanyl, —CH₂-piperidinyl, —CH₂-1-azabicyclo[2.2.1]heptanyl, morpholinyl, —CH₂-oxa-5-azabicyclo[2.2.1]heptan-5-yl, —CH₂-thiopyranyl, —CH₂-6-oxa-212azaspiro[3.4]octanyl, —CH₂-7-oxa-212-azaspiro[3.5]nonanyl, —CH₂-2′,3′-dihydrospiro[cyclopropane-1,1′-indenyl], —CH₂-(2S)-1-azabicyclo[2.2.1]heptan-2-yl, and —CH₂-tetrahydrofuranyl, each optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-hexahydro-IH-pyrrolizinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-hexahydro-3H-pyrrolizin-3-one optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-hexahydro-1H-pyrrolo[2,1-c][1,4]oxazinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-octahydroindolizinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-hexahydropyrrolizine 4(IH)-oxide optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-azetidinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-pyrrolidinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-pyrrolidin-2-one optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-oxetanyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-piperidinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-1-azabicyclo[2.2.1]heptanyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is morpholinyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-oxa-5-azabicyclo[2.2.1]heptan-5-yl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-thiopyranyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-6-oxa-212azaspiro[3.4]octanyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-7-oxa-212-azaspiro[3.5]nonanyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-2′,3′-dihydrospiro[cyclopropane-1,l′-indenyl] optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-(2S)-1-azabicyclo[2.2.1]heptan-2-yl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{12b} is —CH₂-tetrahydrofuranyl optionally substituted with one, two, or three R^{20d}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³). In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{20d} is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{20d} is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{20d} is independently methyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{20d} is independently fluoro.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R² is selected from

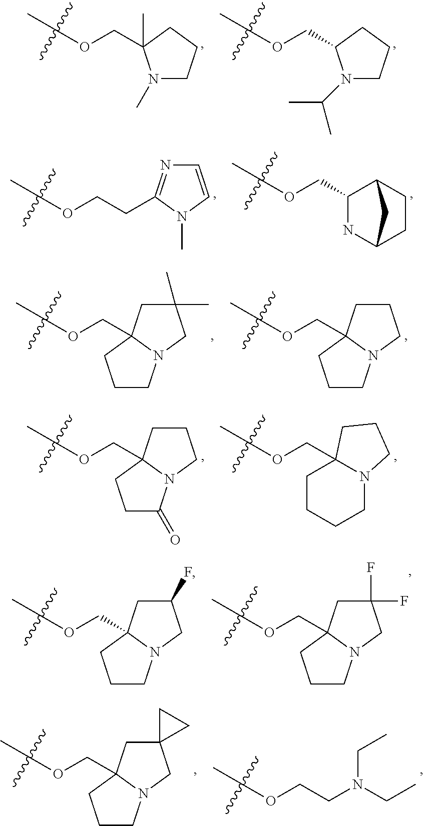

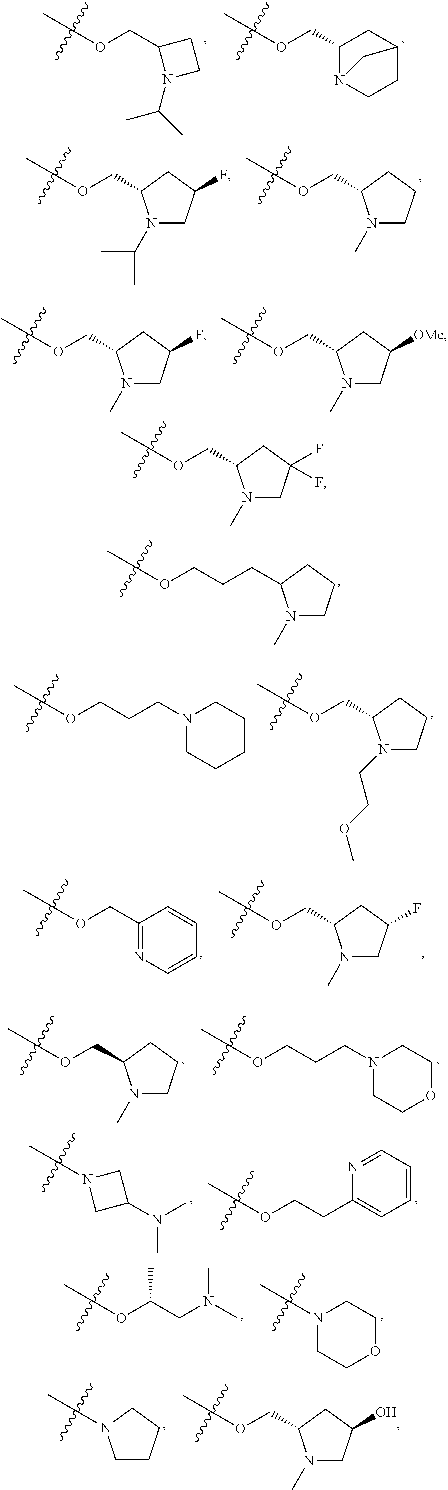

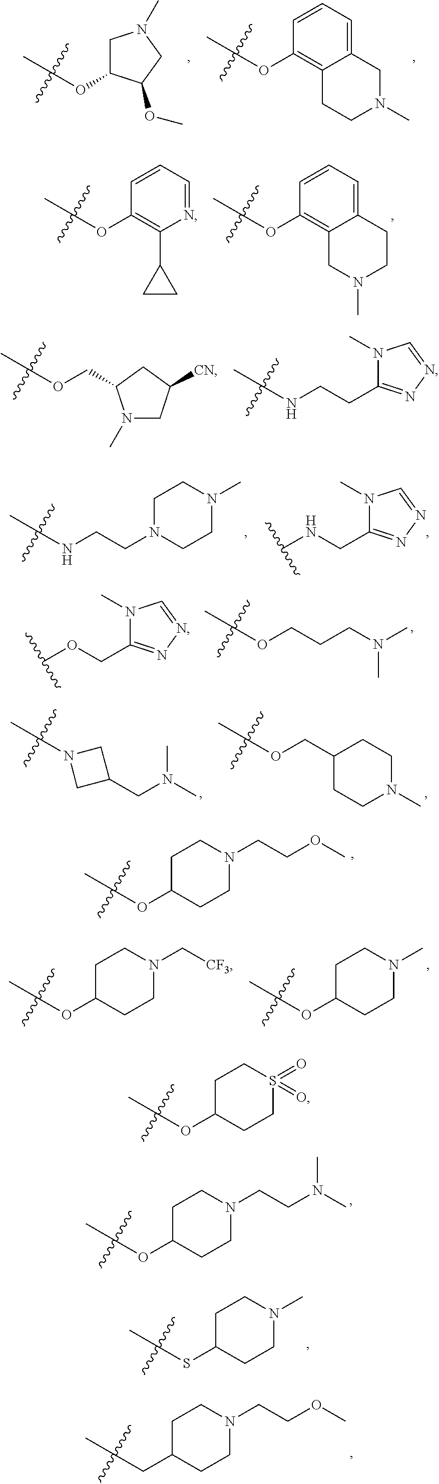

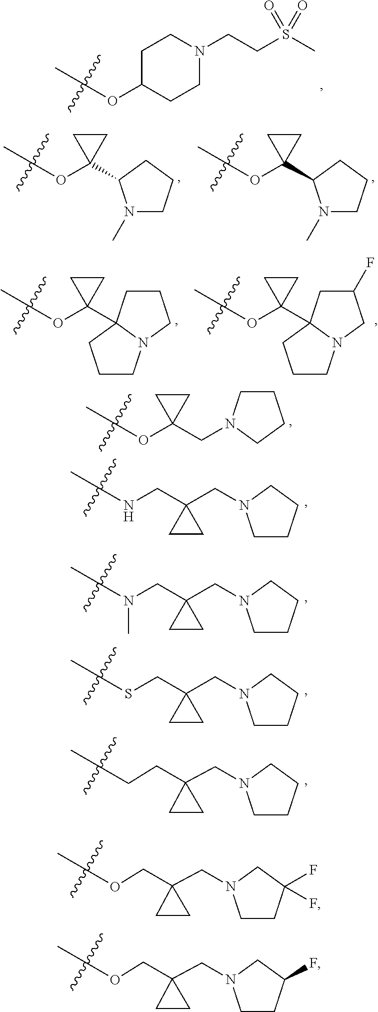

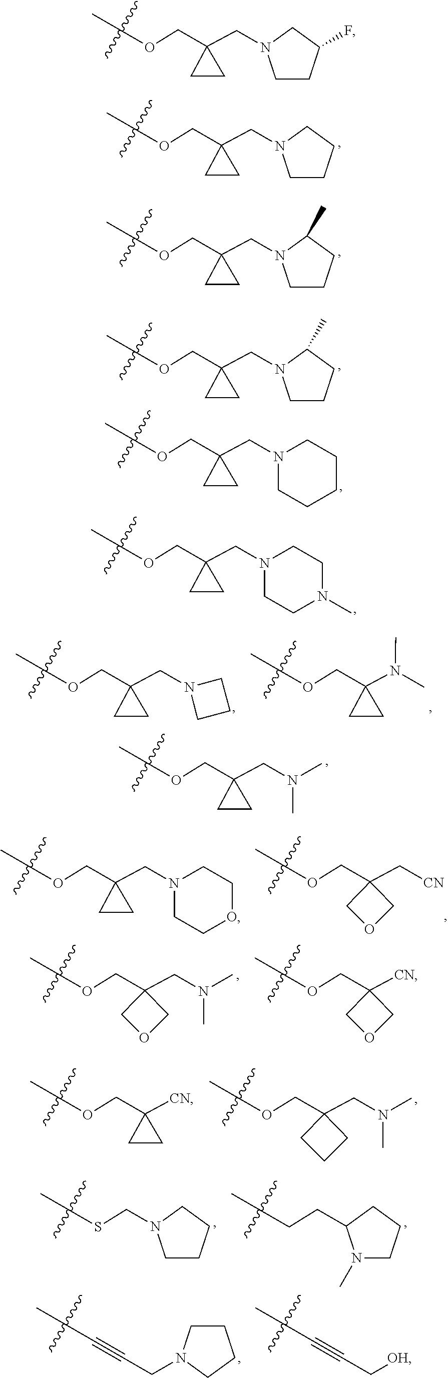

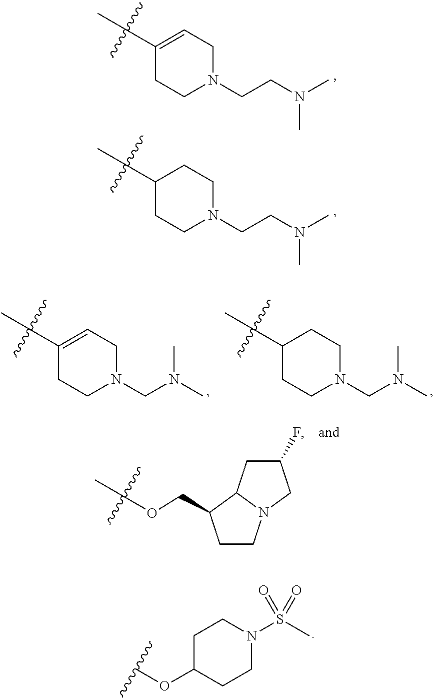

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R² is selected from

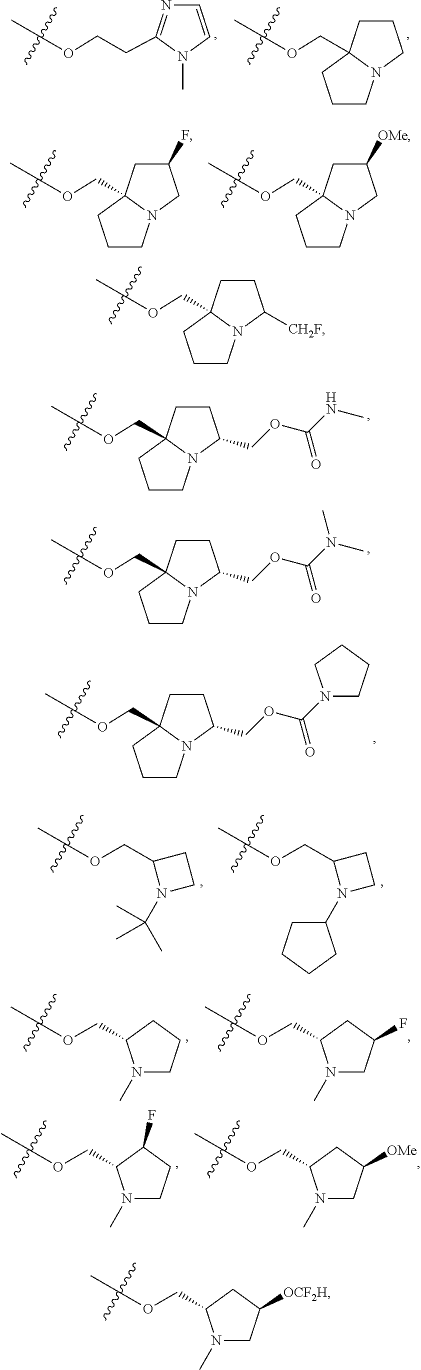

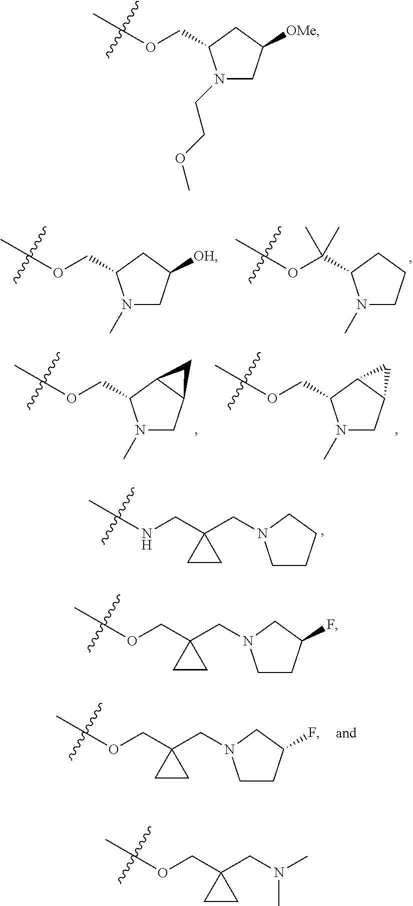

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R² is

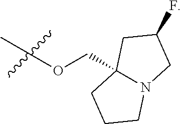 [SMILES: *OC[C@@]12CCCN1C[C@H](F)C2]

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R² is

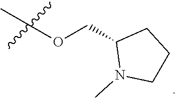 [SMILES: *OC[C@@H]1CCCN1C]

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, J is N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, J is CH(R¹⁷). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, J is C(R¹⁷).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is a bond. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is C₁-C₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —O—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —N(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —NH—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —C(O)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —N(R¹⁴)C(O)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —C(O)N(R¹⁴)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is N(R^{1e}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is C(O)N(R^{1c}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is C(R¹⁶)(R¹⁸). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is CH₂. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is —C(O)NH—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is NH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is NHC(O). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is a bond. In further embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, L¹ is O.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₁₋₆alkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₁₋₆haloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1c} is independently C₂₋₆alkenyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₂₋₆alkynyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₃₋₆cycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₂₋₉heterocycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₆₋₁₀aryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1e} is independently C₁₋₉heteroaryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₁₋₆alkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₁₋₆haloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₂₋₆alkenyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₂₋₆alkynyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₃₋₆cycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₂₋₉heterocycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₆₋₁₀aryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} is independently C₁₋₉heteroaryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₁₋₆alkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₁₋₆haloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₂₋₆alkenyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₂₋₆alkynyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₃₋₆cycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₂₋₉heterocycloalkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₆₋₁₀aryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁸ is independently C₁₋₉heteroaryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} and R^{1g} are joined to form a 4-7 membered heterocycloalkyl ring optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} and R^{1g} are joined to form a 4-7 membered cycloalkyl ring, wherein the 4-7 membered heterocycloalkyl ring or 4-7 membered cycloalkyl ring optionally substituted with one, two, or three R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₂heteroaryl or a fused ring C₂₋₁₂heteroaryl, wherein the bicyclic C₂₋₁₂heteroaryl and fused ring C₂₋₁₂heteroaryl are optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₂heteroaryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₂₋₁₂heteroaryl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₂₋₁₂heteroaryl optionally substituted with one, two, or three R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is selected from:

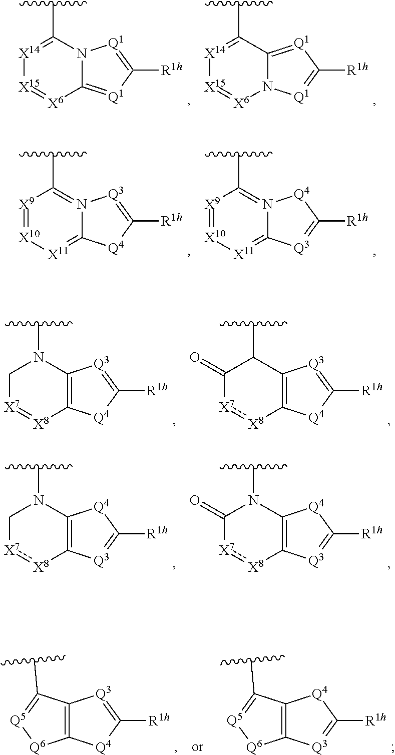

Q¹, Q³, and Q⁵ are independently N or C(R^{1d});
Q⁴ and Q⁶ are independently O, S, C(R^{1a})(R^{1b}), or N(R^{1c});
X¹⁴, X¹⁵, X⁶, X⁹, X¹⁰, and X¹¹ are independently selected from C(R^{1a}) or N;
X⁷ and X⁸ are independently selected from C(R^{1a}), C(R^{1a})(R^{1b}), N, or N(R^{1c});
each R^{1a}, R^{1b}, R^{1d}, R^{1f}, R^{1g}, and R^{1h} are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, and
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

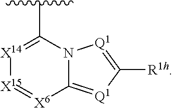

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

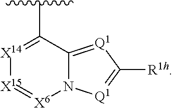

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XIII-1), (XXIII-2), (XXIV-1), or (XXIV-2), or a pharmaceutically acceptable salt or solvate thereof, R¹⁹ is

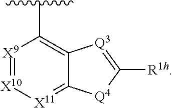

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

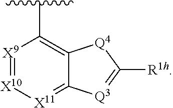

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

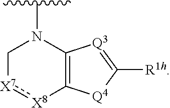

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

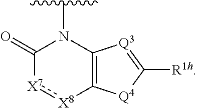

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

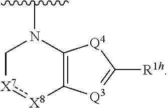

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

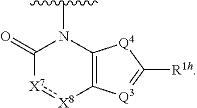

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

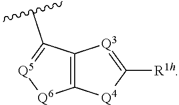

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is

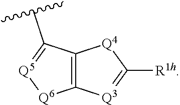

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₄₋₁₂cycloalkyl optionally substituted with one, two, three, four, or five R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₁heterocycloalkyl optionally substituted with one, two, three, four, or five R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a naphthalenyl optionally substituted with one, two, three, four, or five R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₇₋₁₂aryl optionally substituted with one, two, three, four, or five R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₂heteroaryl optionally substituted with one, two, three, four, or five R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₂₋₁₂heteroaryl optionally substituted with one, two, three, four, or five R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₄₋₁₂cycloalkyl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₁heterocycloalkyl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a naphthalenyl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₇₋₁₂aryl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a bicyclic C₂₋₁₂heteroaryl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is a fused ring C₂₋₁₂heteroaryl optionally substituted with one, two, three, four, five, six, or seven R¹ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently halogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —CN. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently C₁₋₆alkyl optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently C₁₋₆haloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —OR¹². In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —SR¹². In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —N(R¹²)(R¹³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —C(O)OR¹². In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —C(O)N(R¹²)(R¹³). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —OH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —SH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —NH₂. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —C(O)OH. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹ⁱ is independently —C(O)NH₂.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q¹, Q³, and Q⁵ are independently N or C(R^{1d}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q¹ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q¹ is independently C(R^{1d}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q³ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q³ is independently C(R^{1d}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁵ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁵ is independently C(R^{1d}).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁴ and Q⁶ are independently O, S, C(R^{1a})(R^{1b}), or N(R^{1c}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁴ is independently O. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁴ is independently S. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁴ is independently C(R^{1a})(R^{1b}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁴ is independently N(R^{1c}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁶ is independently O. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁶ is independently S. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁶ is independently C(R^{1a})(R^{1b}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, Q⁶ is independently N(R^{1c}).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁴, X¹⁵, X⁶, X⁹, X¹⁰, and X¹¹ are independently selected from C(R^{1a}) or N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁴ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁴ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁵ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁵ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁶ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁶ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁹ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁹ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁰ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹⁰ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹¹ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X¹¹ is independently N.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁷ and X⁸ are independently selected from C(R^{1a}), C(R^{1a})(R^{1b}), N, or N(R^{1c}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁷ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁷ is independently C(R^{1a})(R^{1b}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁷ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁷ is independently N(R^{1c}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁸ is independently C(R^{1a}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁸ is independently C(R^{1a})(R^{1b}). In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁸ is independently N. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, X⁸ is independently N(R^{1c}).

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1a}, R^{1b}, R^{1d}, R^{1f}, R^{1g}, and R^{1h} are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ, or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or Rif and R¹⁸ are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1a}, R^{1b}, R^{1d}, R^{1f}, R¹⁸, and R^{1h} are independently selected from hydrogen, halogen, C₁₋₆alkyl, C₁₋₆haloalkyl, —OR¹², —N(R¹²)(R¹³), wherein C₁₋₆alkyl is optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1a}, R^{1b}, R^{1d}, R^{1f}, R^{1g}, and R^{1h} are independently selected from hydrogen, halogen, C₁₋₆alkyl, C₁₋₆haloalkyl, —OH, —NH₂, wherein C₁₋₆alkyl is optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1c} is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{1c} is independently C₁₋₆alkyl optionally substituted with one, two, or three R²⁰ⁱ.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is selected from

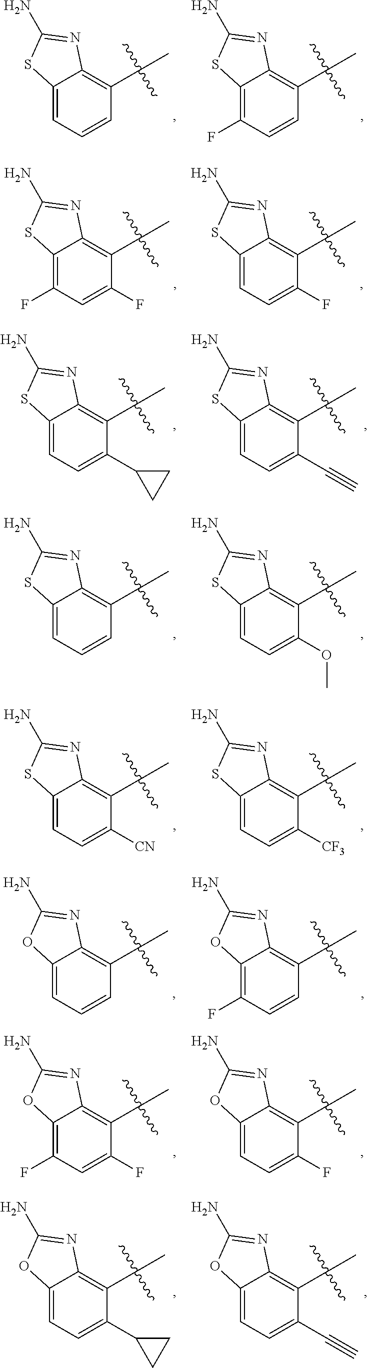

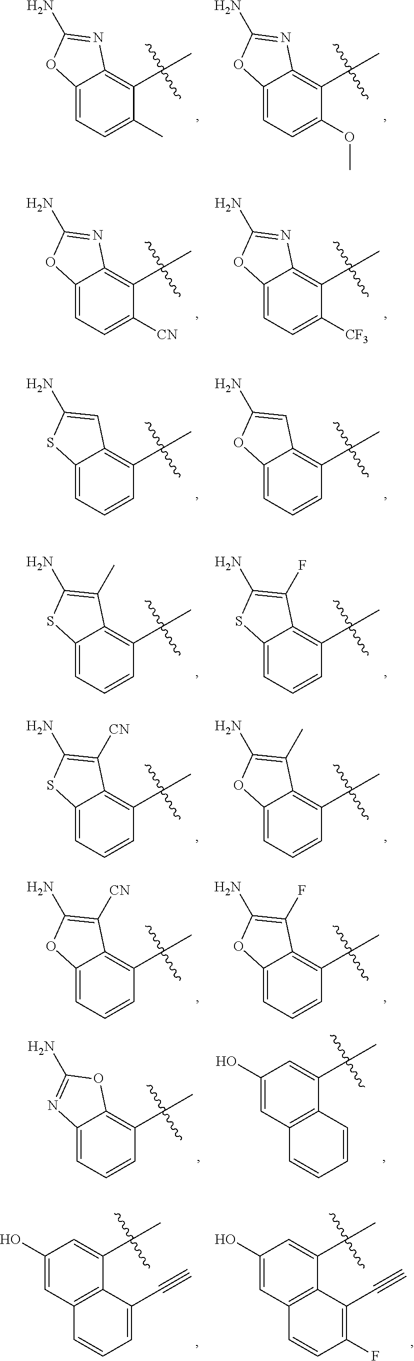

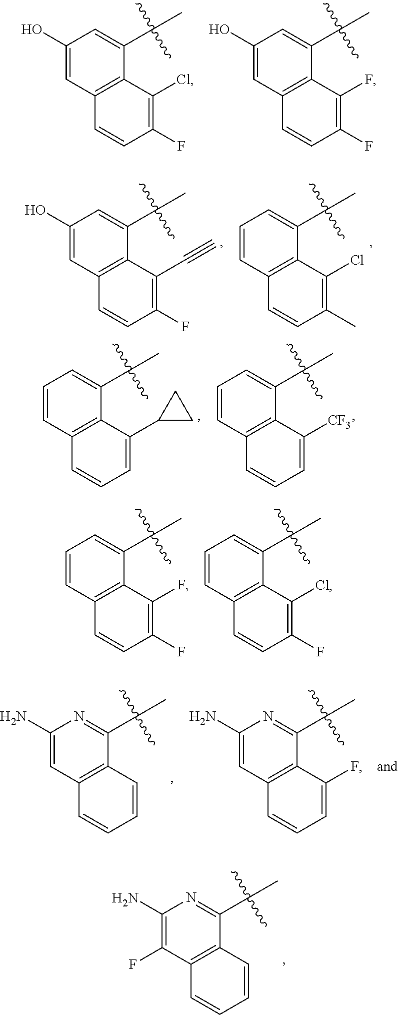

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, p is 0.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each L² is selected from a bond, C₁-C₆alkyl, and —C(O)—. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each L² is a bond. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each L² is C₁-Calkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each L² is —C(O)—.

In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is hydrogen.

In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is a group other than an electrophilic moiety capable of forming a covalent bond with the cysteine residue at position 12 of a KRAS protein. In further embodiments of the subject compound of Formula, or(XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently selected from: —H, —NH₂, —OH, —NH(C₁₋₆ alkyl).

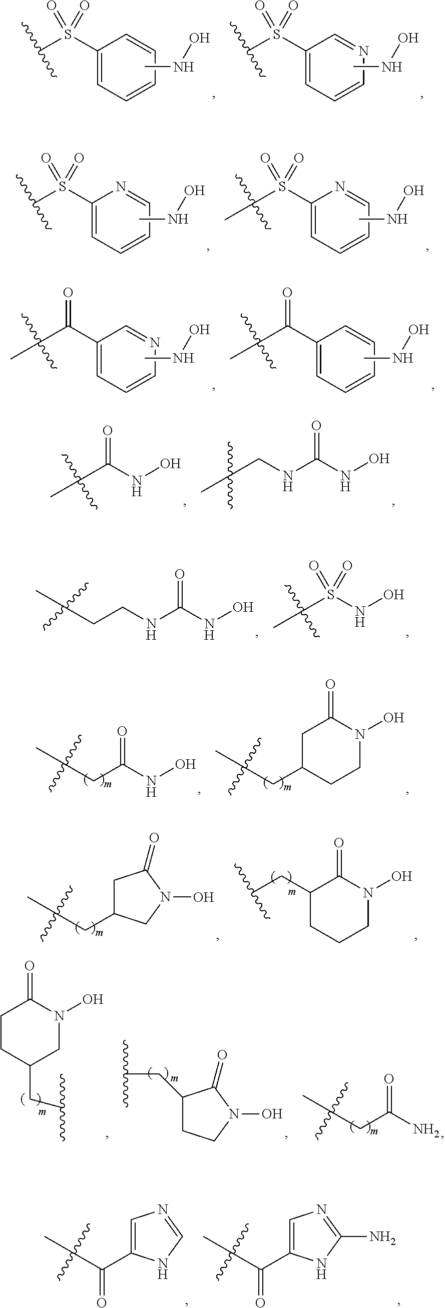

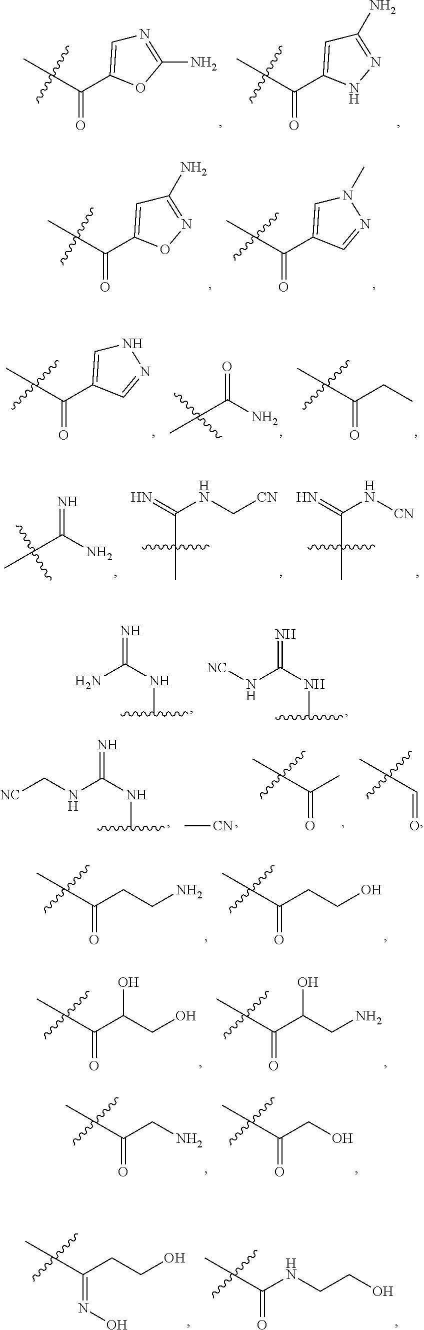

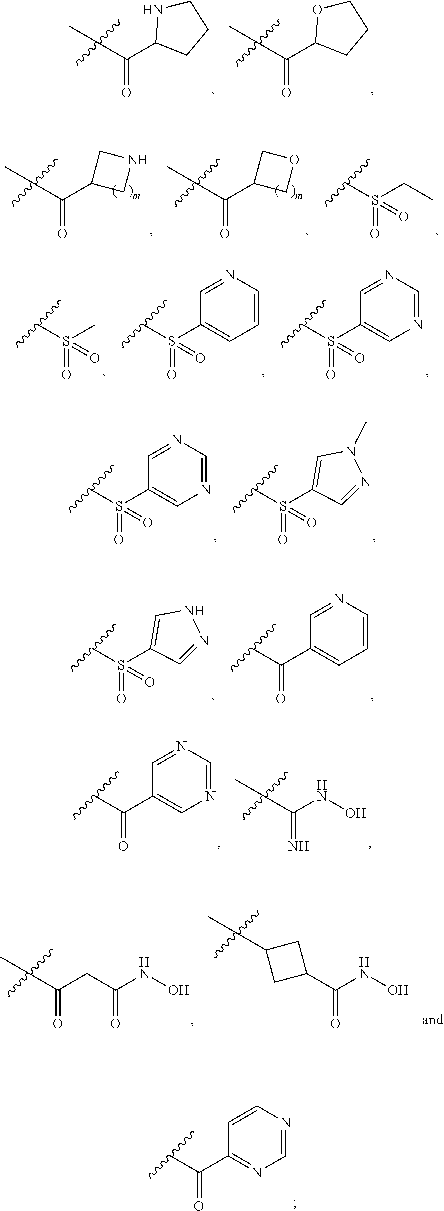

and m, when present, is 0, 1, 2, or 3.

In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, or —CH₂S(O)₂N(R¹²)(R¹³), wherein C₁₋₆alkyl, C₂₋₆alkenyl, C₂₋₆alkynyl, C₃₋₆cycloalkyl, C_{2- 9}heterocycloalkyl, C₆₋₁₀aryl, and C₁₋₉heteroaryl are optionally substituted with one, two, or three R^{20a}. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OH, —SH, —NH₂, —C(O)OH, —OC(O)NH₂, —NHC(O)NH₂, —NHC(O)OH, —NHS(O)₂H, —C(O)H, —S(O)H, —OC(O)H, —C(O)NH₂, —C(O)C(O)NH₂, —NHC(O)H, —S(O)₂H, —S(O)₂NH₂—, S(═O)(═NH)NH₂, —CH₂C(O)NH₂, —CH₂NHC(O)H, —CH₂S(O)₂H, or —CH₂S(O)₂NH₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently halogen. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —CN. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OH. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NH₂. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)OH. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OC(O)NH₂. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)NH₂. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHC(O)OH. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —NHS(O)₂H. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —C(O)NH₂. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently each R⁵ is independently hydrogen. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently oxo. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₁₋₆alkyl optionally substituted with one, two, or three R^{20a}. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₃₋₆cycloalkyl optionally substituted with one, two, or three R^{20a}. In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₂₋₉heterocycloalkyl optionally substituted with one, two, or three R^{20a}. In some embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently C₆₋₁₀aryl optionally substituted with one, two, or three R^{20a}. In further embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently each R⁵ is independently C₁₋₉heteroaryl optionally substituted with one, two, or three R^{20a}. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —OR¹². In embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently —N(R¹²)(R¹³). In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently N(R¹⁴)S(O)₂R¹⁵. In select embodiments of the subject compound of Formula (XXI-1), (XXIa), (XXIb), (XXIc), (XXII-1), (XXIIa-1), (XXIIb-1), (XXIIc), (XXIId), (XXIIe), (XXIIf), (XXIIg), (XXIII-1), or (XXIV-1), or a pharmaceutically acceptable salt or solvate thereof, each R⁵ is independently independently —S(O)₂R¹⁵. In embodiments, each R^{20a} is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵; each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; each R²² is independently selected from H, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl; each R²³ is independently selected from H and C₁₋₆alkyl; each R²⁴ is independently selected from H and C₁₋₆alkyl; each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl. In embodiments, each R^{20a} is independently selected from halogen, —CN, —OR²¹, and —N(R²²)(R²³). In embodiments, each R^{20a} is independently selected from halogen, —CN, —OH, and —NH₂. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments, each R^{20a} is independently selected from C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl. In embodiments, R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, and —CH₂—C₂₋₉heterocycloalkyl, are optionally substituted with one, two, or three R^{20d}. In embodiments, R¹³ is independently selected from hydrogen, C₁₋₆alkyl, and C₁₋₆haloalkyl. In embodiments, R¹⁴ is independently selected from hydrogen, C₁₋₆alkyl, and C₁₋₆haloalkyl. In embodiments, R¹⁵ is independently selected C₁₋₆alkyl, C₂₋₆alkenyl, C₂₋₆alkynyl, C₃₋₆cycloalkyl, and C₂₋₉heterocycloalkyl, wherein C₁₋₆alkyl, C₂₋₆alkenyl, C₂₋₆alkynyl, C₃₋₆cycloalkyl, and C₂₋₉heterocycloalkyl are optionally substituted with one, two, or three R^{20f}.

In some embodiments is a compound of Formula (XXI-2), (XXIa-2), (XXIb-2), (XXIc), (XXII), (XXIIa), (XXIIb), (XXIIc), (XXIId), (XXIIe), (XXIIf), (XXIIg), (XXIII-2), or (XXIV-2), or a pharmaceutically acceptable salt or solvate thereof, wherein each R⁵ is an electrophilic moiety capable of forming a covalent bond with a residue at position 12 of a KRAS protein, e.g., the cysteine residue at position 12. In some embodiments is a compound of Formula (XXI-2), (XXIa-2), (XXIb-2), (XXIc), (XXII), (XXIIa), (XXIIb), (XXIIc), (XXIId), (XXIIe), (XXIIf), (XXIIg), (XXIII-2), or (XXIV-2), or a pharmaceutically acceptable salt or solvate thereof, wherein each R⁵ is an electrophilic moiety capable of forming a covalent bond with the cysteine residue at position 12 of a KRAS protein. In some embodiments is a compound of Formula (XXI-2), (XXIa-2), (XXIb-2), (XXIc), (XXII), (XXIIa), (XXIIb), (XXIIc), (XXIId), (XXIIe), (XXIIf), (XXIIg), (XXIII-2), or (XXIV-2), or a pharmaceutically acceptable salt or solvate thereof, wherein each R⁵ is selected from the group consisting of

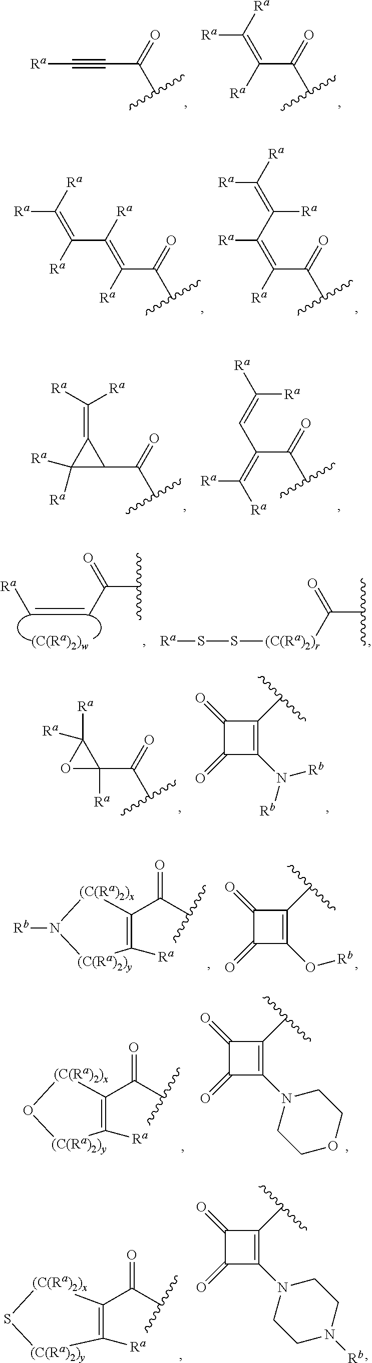

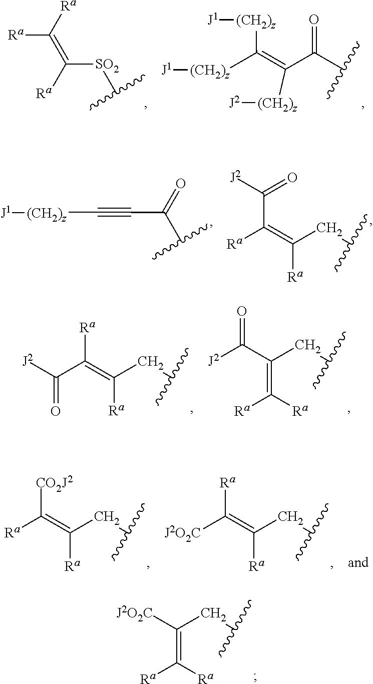

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-(C(R^{b})₂)ᵣ—, (R^{d})(R^{e})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)ᵣ—NR^{b}R^{b}, or —(C(R^{b})₂), —OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R⁴)—, —O—, or a bond; Het is a heterocycle, optionally mono- or di-substituted on carbon or nitrogen with R^{c} and optionally mono-substituted on carbon with —CH₂OR^{b}; wherein the heterocycle is selected from the group consisting of morpholine, thiomorpholine, thiomorpholine S-oxide, thiomorpholine S, S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4. In some embodiments, each R⁵ is selected from the group consisting of:

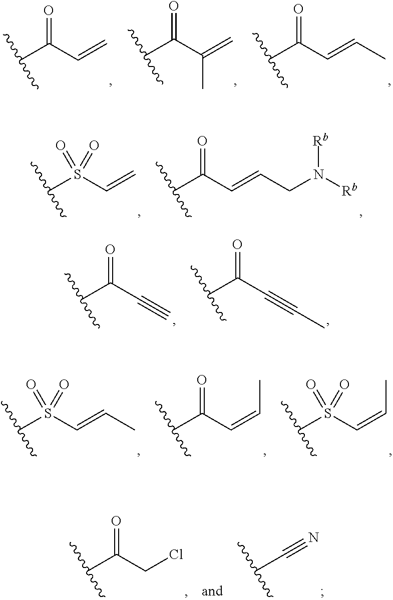

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹² is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹² is independently C₁₋₆alkyl optionally substituted with one, two, or three R^{20d}. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹² is independently C₃₋₆cycloalkyl optionally substituted with one, two, or three R^{20d}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹³ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹³ is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹³ is independently C₁₋₆haloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹³ is independently R¹² and R¹³, together with the nitrogen to which they are attached, form a C₂₋₉heterocycloalkyl ring optionally substituted with one, two, or three R^{20c}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹⁴ is independently hydrogen. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹⁴ is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹⁴ is independently C₁₋₆haloalkyl.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R¹⁵ is independently C₁₋₆alkyl optionally substituted with one, two, or three R^{20f}.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OH, —SH, —NH₂, —C(O)OH, —C(O)NH₂, —C(O)C(O)NH₂, —OC(O)NH₂, —NHC(O)NH₂, —NHC(O)OH, —NHC(O)H, —NHS(O)₂H, —C(O)H, —S(O)₂H, —S(O)₂NH₂, —OCH₂C(O)OH, and —OC(O)H, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OH, —SH, —NH₂, —C(O)OH, —C(O)NH₂, —C(O)C(O)NH₂, —OC(O)NH₂, —NHC(O)NH₂, —NHC(O)OH, —NHC(O)H, —NHS(O)₂H, —C(O)H, —S(O)₂H, —S(O)₂NH₂, and —OC(O)H.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, each R²¹ is independently selected from H, C₁₋₆alkyl, C₁₋₆haloalkyl, C₂₋₆alkenyl, C₂₋₆alkynyl, C₃₋₆cycloalkyl, C₂— heterocycloalkyl, C₆₋₁₀aryl, and C₁₋₉heteroaryl;

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently H. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₁₋₆haloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₂₋₆alkenyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₂₋₆alkynyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₃₋₆cycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₂₋₉heterocycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₆₋₁₀aryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²² is independently C₁₋₉heteroaryl.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²³ is independently H. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²³ is independently C₁₋₆alkyl.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁴ is independently H. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁴ is independently C₁₋₆alkyl.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₁₋₆alkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₂₋₆alkenyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₂₋₆alkynyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₃₋₆cycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₂₋₉heterocycloalkyl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₆₋₁₀aryl. In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R²⁵ is independently C₁₋₉heteroaryl.

In an aspect is provided a compound of Formula (XXIII-1), or a pharmaceutically acceptable salt or solvate thereof:

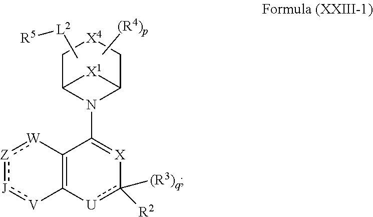

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁴)C═N—OR⁴, CH₂C═NN(R⁴)(R⁶), C(R⁴)(R⁶)C(O)N(R⁴), C(R⁴)(R⁴)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;

R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ,
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d},
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12c};
L³ is selected from C₁-C₆alkyl, C₂₋₆alkenyl, C₂₋₆alkynyl, —O—, —N(R¹⁴)—, and —S—;
R^{12c} is independently selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉ heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXIII-2), or a pharmaceutically acceptable salt or solvate thereof:

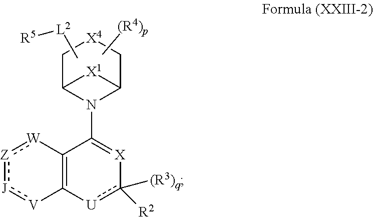

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴),) C(R⁴)(R⁴)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};

J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or Rif and R¹⁸ are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;
R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ,
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12c}.
L³ is selected from C₁-C₆alkyl, C₂₋₆alkenyl, C₂₋₆alkynyl, —O—, —N(R¹⁴)—, and —S—;
R^{12c} is independently selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C_{1- 9}heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXIV-1), or a pharmaceutically acceptable salt or solvate thereof:

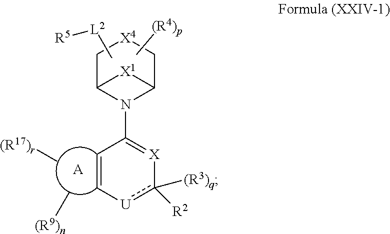

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴);
N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶),) C(R⁴)(R⁶)O, C(R⁴)(R⁴)OC(R⁴)(R⁶), C(R⁴)(R⁶)S,))C(R⁴)(R⁴)SC(R⁴)(R⁴), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁴)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C—N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R¹⁸ is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;
R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is provided a compound of Formula (XXIV-2), or a pharmaceutically acceptable salt or solvate thereof:

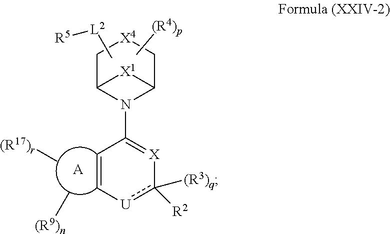

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴); N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R 12b;
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R¹)(R^{1g})O, C(R¹⁶)(R^{1g})N(R^{1c}), and C(R^{1f})(R¹⁸);
each R^{1c}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₅alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃-cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₅alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments of the subject compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof, R¹⁹ is selected from

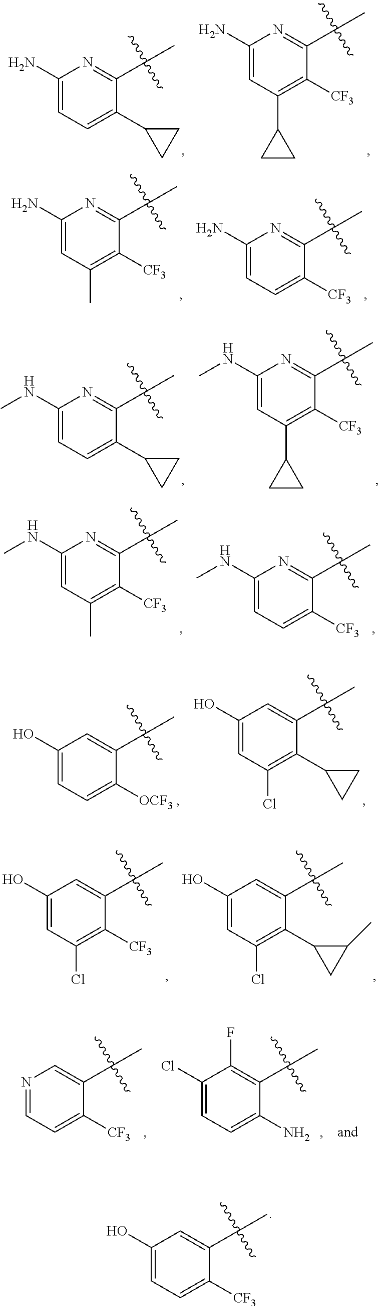

In embodiments,

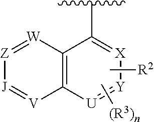

is selected from

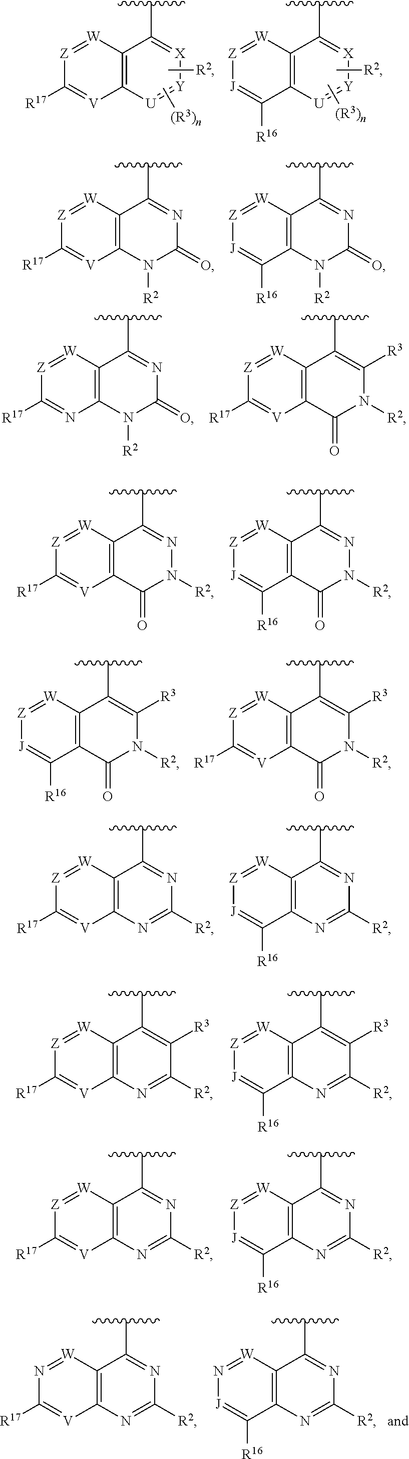

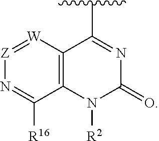

In embodiments,

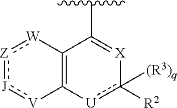

is selected from

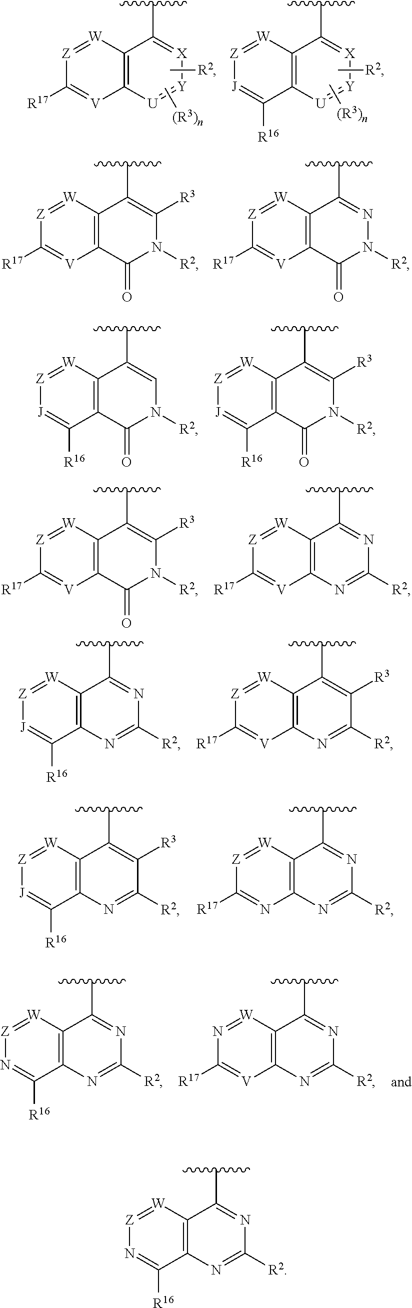

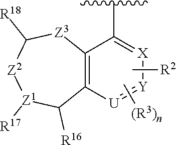

is selected from

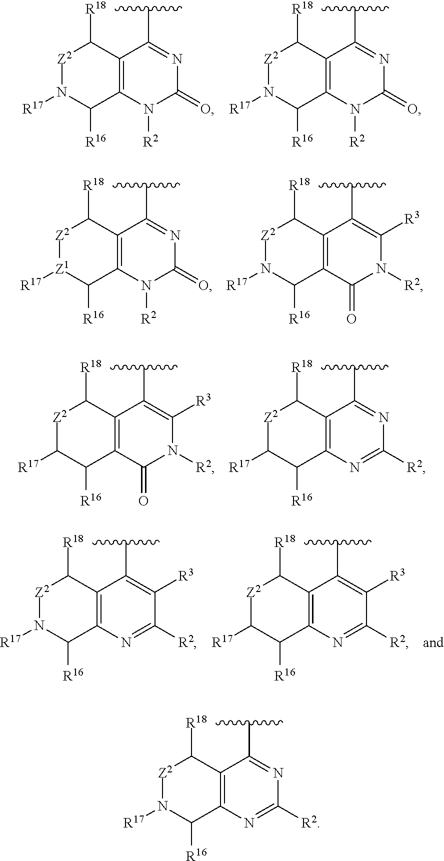

In embodiments,

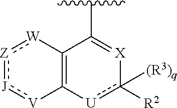

is selected from

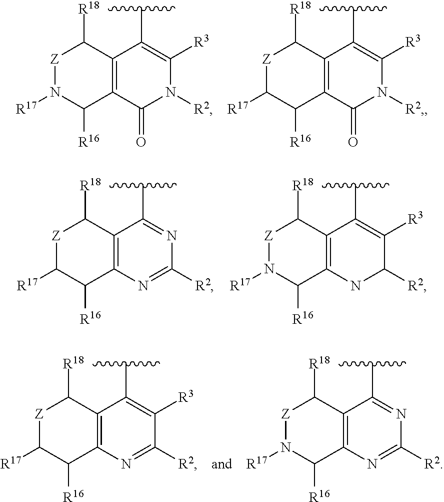

In embodiments,

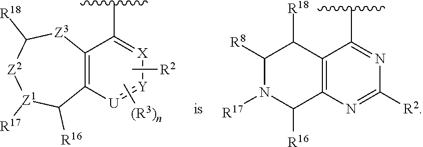

In embodiments,

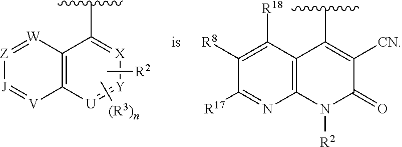

In embodiments,

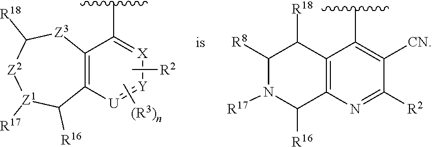

In embodiments,

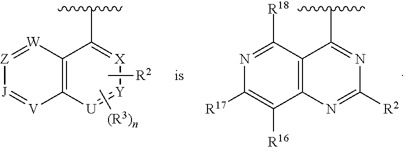

In embodiments,

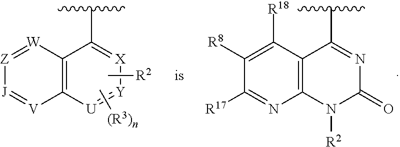

In embodiments,

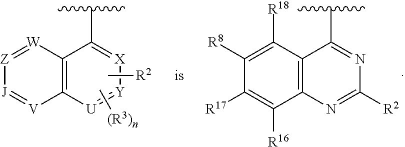

In embodiments.

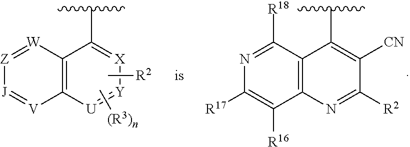

In embodiments,

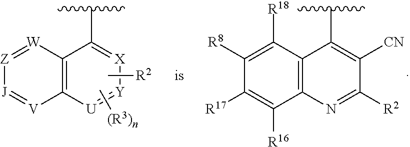

In embodiments,

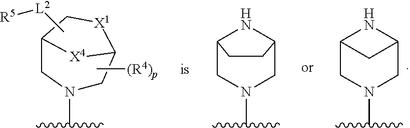

In embodiments,

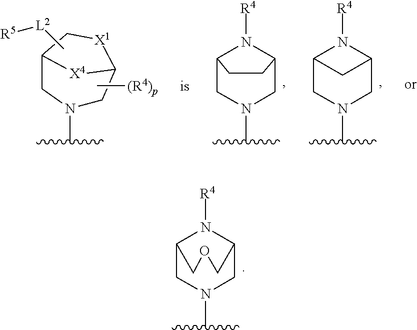

In embodiments,

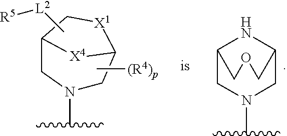

In embodiments,

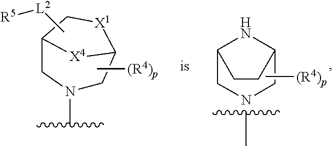

and p is independently an integer from 0 to 4.

In embodiments,

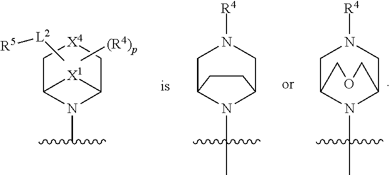

In embodiments,

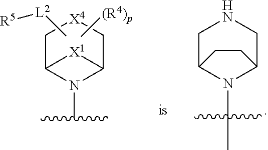

In embodiments,

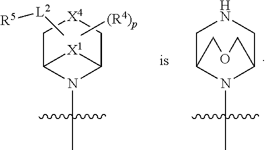

In embodiments,

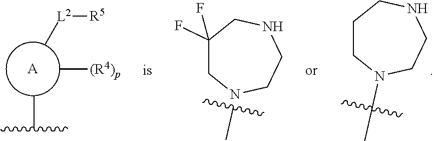

In embodiments,

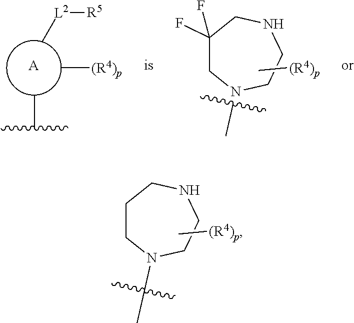

and p is independently an integer from 0 to 4. In embodiments,

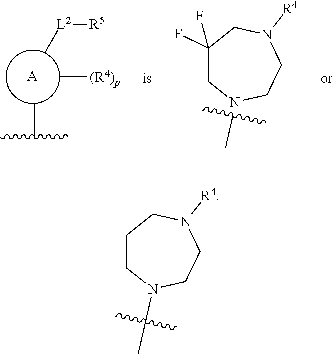

In embodiments,

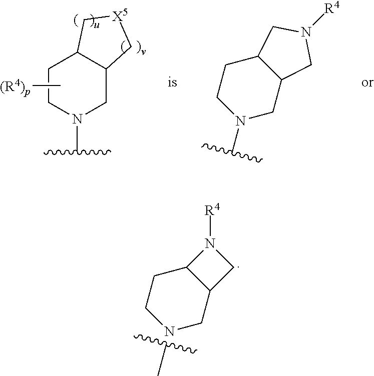

In embodiments,

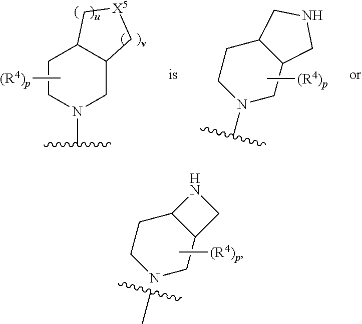

and p is independently an integer from 0 to 4. In embodiments,

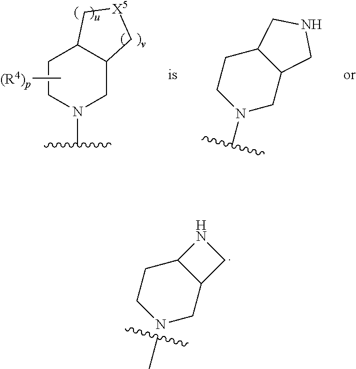

In some embodiments,

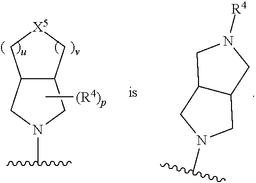

In some embodiments,

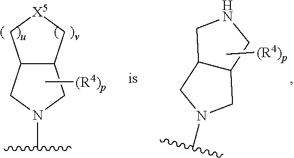

and p is independently an integer from 0 to 4. In some embodiments,

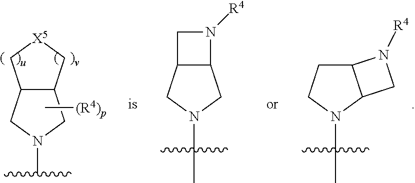

In some embodiments,

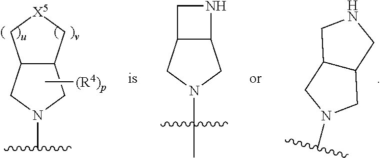

In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (Ille-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 0. In embodiments of a compound of Formula (I-1), (I-2), (I′), (I″-1), (I′″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (INIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 1. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 2. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 3. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 6. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 7. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (INIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 8. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 9. In embodiments of a compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is 10. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 2 to 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 2 to 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 2 to 3. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 3 to 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (INIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 3 to 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 4 to 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 1 to 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 1 to 3. In embodiments of a compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer from 1 to 2. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 2, 3, 4, and 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 2, 3, and 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 2 and 3. In embodiments of a compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 3, 4, and 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 4 and 5. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 1, 2, 3, and 4. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (lIli-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 1, 2, and 3. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (Ile), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 2, 1 and 2. In embodiments of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), p is an integer selected from 3 and 4.

In embodiments of the compound, or a pharmaceutically acceptable salt or solvate thereof, R¹⁷ is:

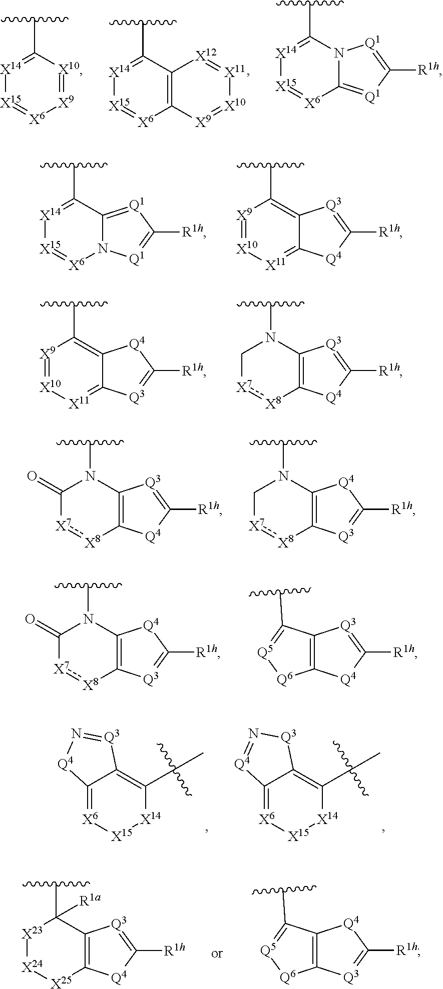

Q¹, Q³, and Q⁵ are independently N or C(R^{1d});
Q⁴ and Q⁶ are independently O, S, C(R^{1a})(R^{1b}), or N(R^{1c});
X¹⁴, X¹⁵, X⁶, X⁹, X¹⁰, X¹¹, and X¹² are independently selected from C(R^{1a}) or N;
X⁷ and X⁸ are independently selected from C(R^{1a}), C(R^{1a})(R^{1b}), N, or N(R^{1c});
X²³ is selected from a bond, C(R^{1a})(R^{1b}), C(R^{1a})(R^{1b})C(R^{1a})(R^{1b}), C(R^{1a})(R^{1b})N(R^{1c}), and N(R^{1c});
X²⁴ and X²⁵ are independently selected from a bond, C(R^{1a})(R^{1b}), and N(R^{1c});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ, or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments, R^{1h} is selected from hydrogen, C₁₋₆alkyl, C₁₋₆haloalkyl, C₂₋₆alkenyl, C₂₋₆alkynyl, —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —C(O)R¹⁵, —OC(O)R¹⁵, —C(O)N(R¹²)(R¹³), —C(O)C(O)N(R¹²)(R¹³), —N(R¹⁴)C(O)R¹⁵, —CH₂C(O)N(R¹²)(R¹³), and —CH₂N(R¹⁴)C(O)R¹⁵, wherein C₁₋₆alkyl, C₂₋₆alkenyl, and C₂₋₆alkynyl are optionally substituted with one, two, or three R²⁰ⁱ.

In embodiments, R^{1h} is selected from hydrogen and —N(R¹²)(R¹³). In embodiments, R^{1h} is hydrogen. In embodiments, R^{1h} is —N(R¹²)(R¹³). In embodiments, R^{1h} is —NH₂.

In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, —CN, C₁₋₆alkyl, and C₁₋₆haloalkyl, wherein C₁₋₆alkyl is optionally substituted with one, two, or three R²⁰ⁱ. In embodiments, R^{1d} is independently hydrogen. In embodiments, R^{1d} is independently —CN.

In some embodiments, R¹⁷ is selected from:

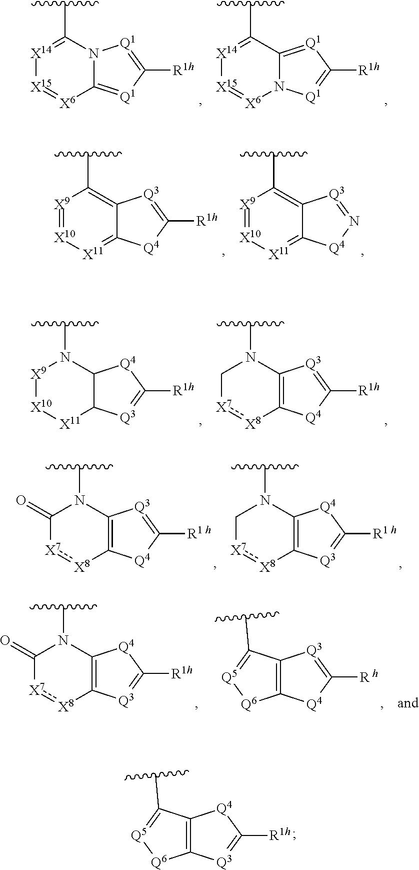

Q¹, Q³, and Q⁵ are independently N or C(R^{1d});
Q⁴ and Q⁶ are independently O, S, C(R^{1a})(R^{1b}), or N(R^{1c});
X¹⁴, X¹⁵, X⁶, X⁹, X¹⁰, and X¹¹ are independently selected from C(R^{1a}) or N;
X⁷ and X⁸ are independently selected from C(R^{1a}), C(R^{1a})(R^{1b}), N, or N(R^{1c});
each R^{1a}, R^{1b}, R^{1d}, R^{1f}, R^{1g}, and R^{1h} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; and each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In embodiments, R^{1h} is selected from hydrogen, C₁₋₆alkyl, C₁₋₆haloalkyl, C₂₋₆alkenyl, C₂₋₆alkynyl, —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —C(O)R¹⁵, —OC(O)R¹⁵, —C(O)N(R¹²)(R¹³), —C(O)C(O)N(R¹²)(R¹³), —N(R¹⁴)C(O)R¹⁵, —CH₂C(O)N(R¹²)(R¹³), and —CH₂N(R¹⁴)C(O)R¹⁵, wherein C₁₋₆alkyl, C₂₋₆alkenyl, and C₂₋₆alkynyl are optionally substituted with one, two, or three R²⁰ⁱ.

In embodiments, R^{1h} is selected from hydrogen and —N(R¹²)(R¹³). In embodiments, R^{1h} is hydrogen. In embodiments, R^{1h} is —N(R¹²)(R¹³). In embodiments, R^{1h} is —NH₂.

In embodiments, R^{1a} is independently hydrogen. In embodiments, R^{1a} is independently halogen. In embodiments, R^{1a} is independently —CN. In embodiments, R^{1a} is independently C₁₋₆alkyl optionally substituted with one, two, or three R²⁰ⁱ. In embodiments, R^{1a} is independently C₁₋₆haloalkyl. In embodiments, R^{1a} is independently C₂₋₆alkenyl optionally substituted with one, two, or three R²⁰ⁱ. In embodiments, R^{1a} is independently C₂₋₆alkynyl optionally substituted with one, two, or three R²⁰ⁱ. In embodiments, R^{1a} is independently C₃₋₁₀cycloalkyl optionally substituted with one, two, or three R²⁰ⁱ. In embodiments, R^{1a} is independently C₃₋₆cycloalkyl optionally substituted with one, two, or three R²⁰ⁱ. In embodiments, R^{1a} is independently C₂₋₉heterocycloalkyl optionally substituted with one, two, or three R²⁰ⁱ. In embodiments, R^{1a} is independently C₆₋₁₀aryl optionally substituted with one, two, or three R²⁰ⁱ. In embodiments, R^{1a} is independently C₁₋₉heteroaryl optionally substituted with one, two, or three R²⁰ⁱ. In embodiments, R^{1a} is independently —OR¹². In embodiments, R^{1a} is independently —SR¹². In embodiments, R^{1a} is independently —N(R¹²)(R¹³). In embodiments, R^{1a} is independently —C(O)OR¹². In embodiments, R^{1a} is independently —OC(O)N(R¹²)(R¹³). In embodiments, R^{1a} is independently —N(R¹⁴)C(O)N(R¹²)(R¹³). In embodiments, R^{1a} is independently —N(R¹⁴)C(O)OR¹⁵. In embodiments, R^{1a} is independently —N(R¹⁴)S(O)₂R¹⁵. In embodiments, R^{1a} is independently —C(O)R¹⁵. In embodiments, R^{1a} is independently —S(O)R¹⁵. In embodiments, R^{1a} is independently —OC(O)R¹⁵. In embodiments, R^{1a} is independently —C(O)N(R¹²)(R¹³). In embodiments, R^{1a} is independently —C(O)C(O)N(R¹²)(R¹³). In embodiments, R^{1a} is independently —N(R¹⁴)C(O)R¹⁵. In embodiments, R^{1a} is independently —S(O)₂R¹⁵. In embodiments, R^{1a} is independently —S(O)₂N(R¹²)(R¹³). In embodiments, R^{1a} is independently —S(═O)(═NH)N(R¹²)(R¹³). In embodiments, R^{1a} is independently —CH₂C(O)N(R¹²)(R¹³). In embodiments, R^{1a} is independently —CH₂N(R¹⁴)C(O)R¹⁵. In embodiments, R^{1a} is independently —CH₂S(O)₂R¹⁵. In embodiments, R^{1a} is independently —CH₂S(O)₂N(R¹²)(R¹³). In embodiments, R^{1a} is independently —OH. In embodiments, R^{1a} is independently —SH. In embodiments, R^{1a} is independently —NH₂. In embodiments, R^{1a} is independently —C(O)OH. In embodiments, R^{1a} is independently —OC(O)NH₂. In embodiments, R^{1a} is independently —N(H)C(O)NH₂. In embodiments, R^{1a} is independently —N(H)C(O)OH. In embodiments, R^{1a} is independently —N(H)S(O)₂CH₃. In embodiments, R^{1a} is independently —C(O)CH₃. In embodiments, R^{1a} is independently —S(O)CH₃. In embodiments, R^{1a} is independently —OC(O)CH₃. In embodiments, R^{1a} is independently —C(O)NH₂. In embodiments, R^{1a} is independently —C(O)C(O)NH₂. In embodiments, R^{1a} is independently —N(H)C(O)CH₃. In embodiments, R^{1a} is independently —S(O)₂CH₃. In embodiments, R^{1a} is independently —S(O)₂NH₂. In embodiments, R^{1a} is independently —S(—O)(—NH)NH₂. In embodiments, R^{1a} is independently —CH₂C(O)NH₂. In embodiments, R^{1a} is independently —CH₂N(H)C(O)CH₃. In embodiments, R^{1a} is independently —CH₂S(O)₂CH₃. In embodiments, R^{1a} is independently —CH₂S(O)₂NH₂.

In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}. In embodiments, R^{1d} is independently selected from hydrogen, —CN, C₁₋₆alkyl, and C₁₋₆haloalkyl, wherein C₁₋₆alkyl is optionally substituted with one, two, or three R²⁰ⁱ. In embodiments, R^{1d} is independently hydrogen. In embodiments, R^{1d} is independently —CN.

In embodiments, R¹⁷ is

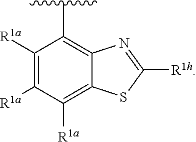

In embodiments, R¹⁷ is

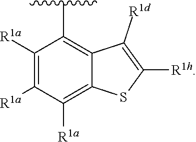

In embodiments, R¹⁷ is

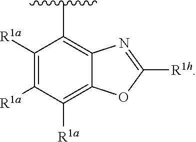

In embodiments, R¹⁷ is

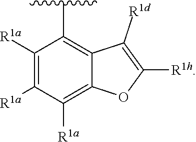

In embodiments, R¹⁷ is

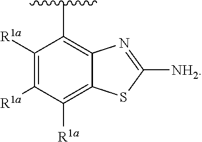

In embodiments, R¹⁷ is

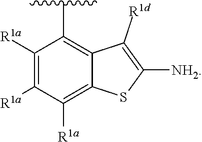

In embodiments, R¹⁷ is

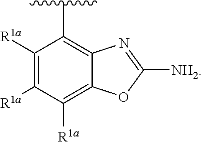

In embodiments, R¹⁷ is

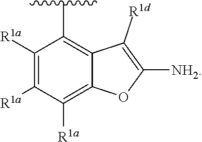

In embodiments, R¹⁷ is

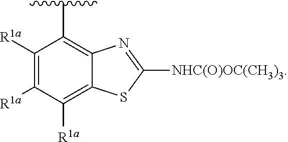

In embodiments, R¹⁷ is

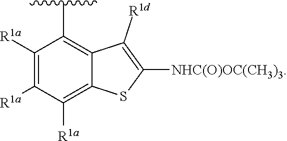

In embodiments, R¹⁷ is

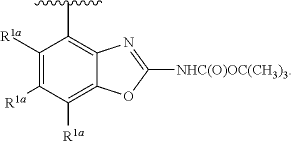

In embodiments, R¹⁷ is

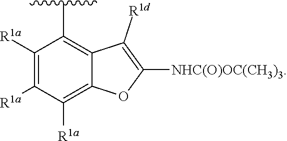

In embodiments, R¹⁷ is

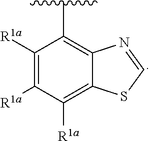

In embodiments, R¹⁷ is

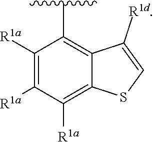

In embodiments, R¹⁷ is

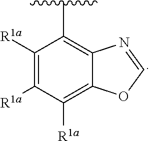

In embodiments, R¹⁷ is

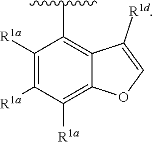

In embodiments, R¹⁷ is

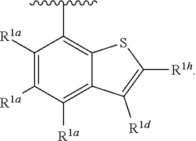

In embodiments, R¹⁷ is

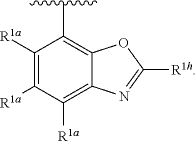

In embodiments, R¹⁷ is

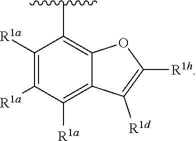

In embodiments, R¹⁷ is

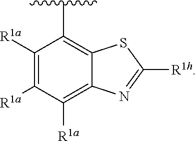

In embodiments, R¹⁷ is

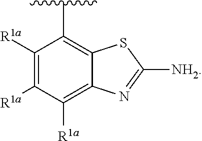

In embodiments, R¹⁷ is

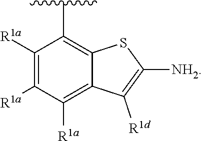

In embodiments, R¹⁷ is

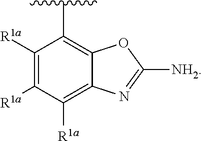

In embodiments, R¹⁷ is

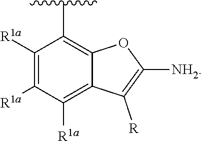

In embodiments, R¹⁷ is

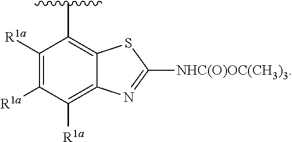

In embodiments, R¹⁷ is

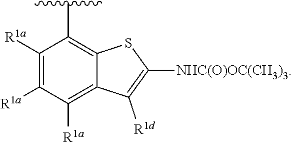

In embodiments, R¹⁷ is

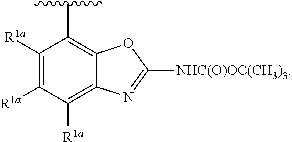

In embodiments, R¹⁷ is

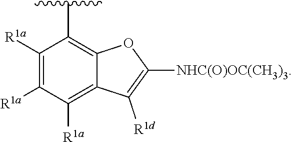

In embodiments, R¹⁷ is

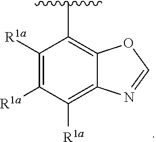

In embodiments, R¹⁷ is

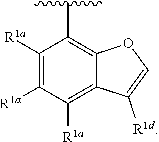

In embodiments, R¹⁷ is

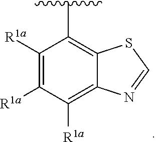

In embodiments, R¹⁷ is

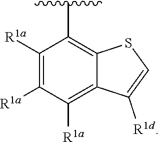

In some embodiments, R¹⁷ is

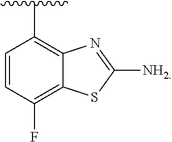 [SMILES: *c1ccc(F)c2sc(N)nc12]

In some embodiments, R¹⁷ is

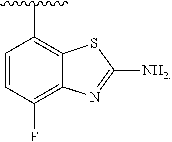 [SMILES: *c1ccc(F)c2nc(N)sc12]

In some embodiments, R¹⁷ is

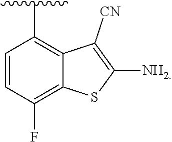 [SMILES: *c1ccc(F)c2sc(N)c(C#N)c12]

In some embodiments, R¹⁷ is

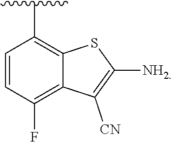 [SMILES: *c1ccc(F)c2c(C#N)c(N)sc12]

In some embodiments, R¹⁷ is

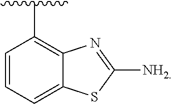 [SMILES: *c1cccc2sc(N)nc12]

In some embodiments, R¹⁷ is

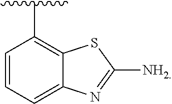 [SMILES: *c1cccc2nc(N)sc12]

In some embodiments, R¹⁷ is

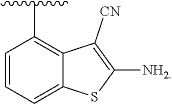 [SMILES: *c1cccc2sc(N)c(C#N)c12]

In some embodiments, R¹⁷ is

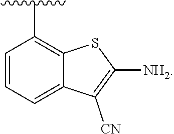 [SMILES: *c1cccc2c(C#N)c(N)sc12]

In some embodiments, R¹⁷ is

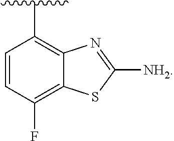 [SMILES: *c1ccc(F)c2sc(N)nc12]

In some embodiments, R¹⁷ is

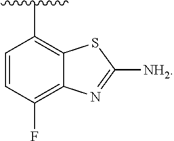 [SMILES: *c1ccc(F)c2nc(N)sc12]

In some embodiments, R¹⁷ is

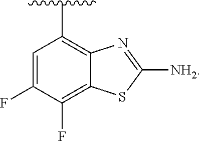 [SMILES: *c1cc(F)c(F)c2sc(N)nc12]

In some embodiments, R¹⁷ is

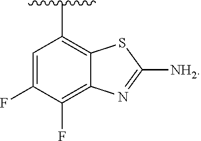 [SMILES: *c1cc(F)c(F)c2nc(N)sc12]

In some embodiments, R¹⁷ is

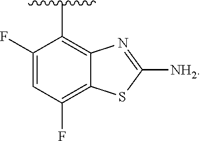 [SMILES: *c1c(F)cc(F)c2sc(N)nc12]

In some embodiments, R¹⁷ is

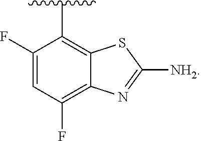 [SMILES: *c1c(F)cc(F)c2nc(N)sc12]

In some embodiments, R¹⁷ is

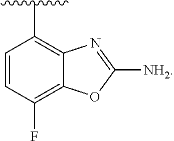 [SMILES: *c1ccc(F)c2oc(N)nc12]

In some embodiments, R¹⁷ is

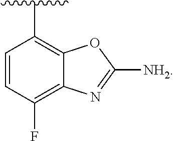 [SMILES: *c1ccc(F)c2nc(N)oc12]

In some embodiments, R¹⁷ is

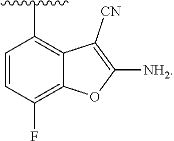 [SMILES: *c1ccc(F)c2oc(N)c(C#N)c12]

In some embodiments, R¹⁷ is

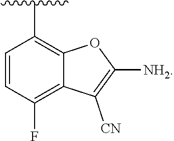 [SMILES: *c1ccc(F)c2c(C#N)c(N)oc12]

In some embodiments, R¹⁷ is

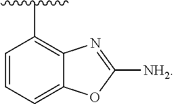 [SMILES: *c1cccc2oc(N)nc12]

In some embodiments, R¹⁷ is

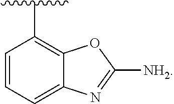 [SMILES: *c1cccc2nc(N)oc12]

In some embodiments, R¹⁷ is

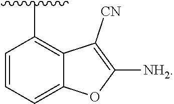 [SMILES: *c1cccc2oc(N)c(C#N)c12]

In some embodiments, R¹⁷ is

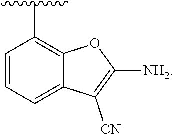 [SMILES: *c1cccc2c(C#N)c(N)oc12]

In some embodiments, R¹⁷ is

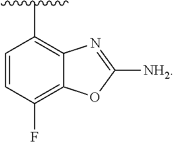 [SMILES: *c1ccc(F)c2oc(N)nc12]

In some embodiments, R¹⁷ is

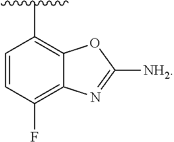 [SMILES: *c1ccc(F)c2nc(N)oc12]

In some embodiments, R¹⁷ is

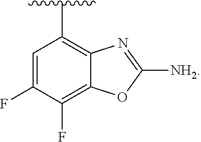 [SMILES: *c1cc(F)c(F)c2oc(N)nc12]

In some embodiments, R¹⁷ is

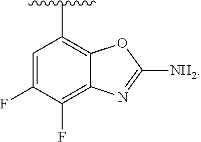 [SMILES: *c1cc(F)c(F)c2nc(N)oc12]

In some embodiments, R¹⁷ is

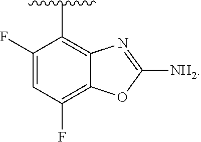 [SMILES: *c1c(F)cc(F)c2oc(N)nc12]

In some embodiments; R¹⁷ is

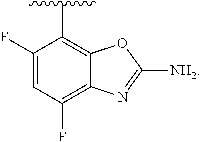 [SMILES: *c1c(F)cc(F)c2nc(N)oc12]

In some embodiments, R¹⁷ is selected from:

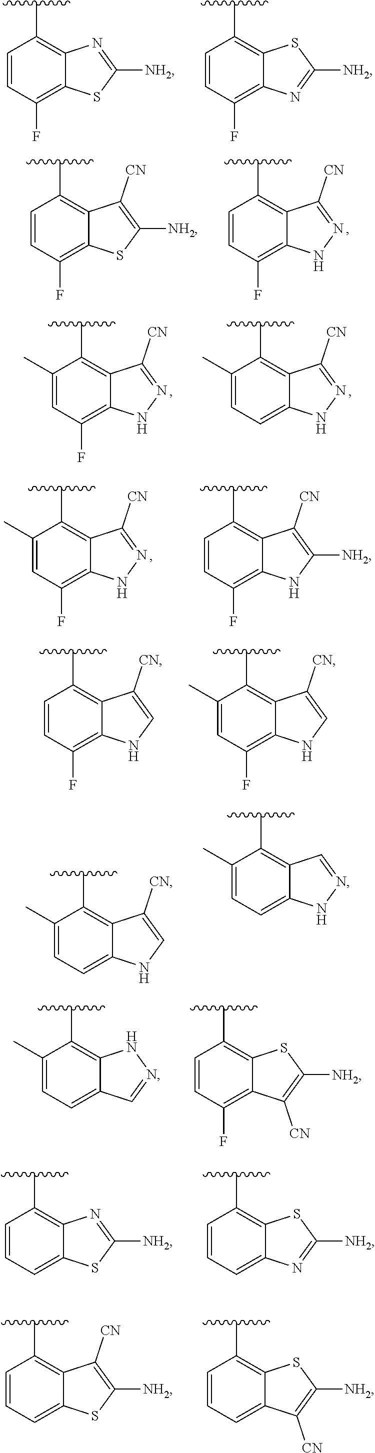

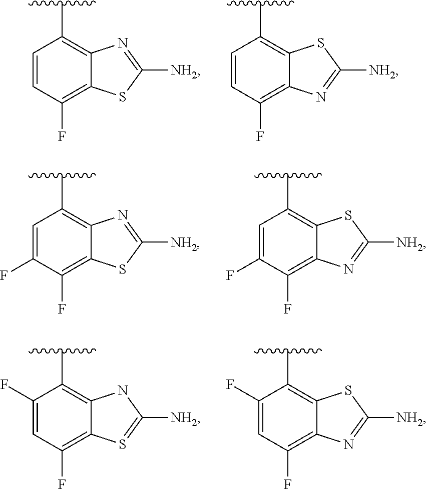

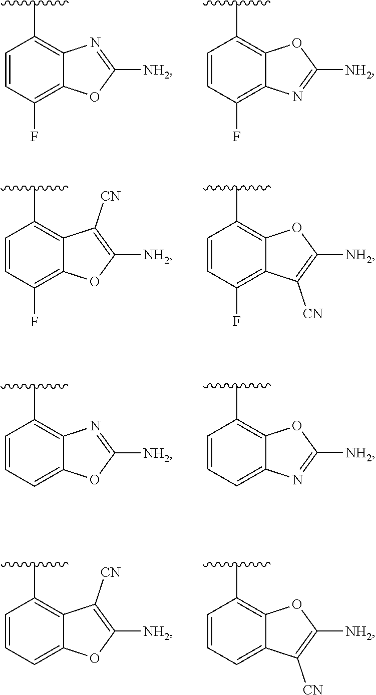

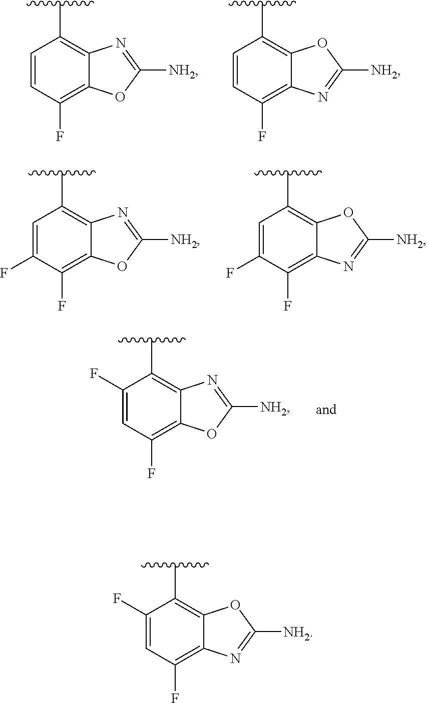

In some embodiments of the compound, or a pharmaceutically acceptable salt or solvate thereof, R¹⁷ is:

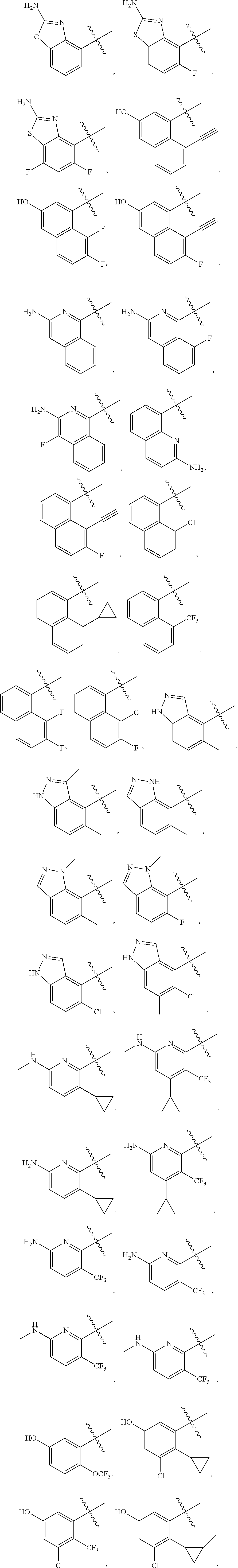

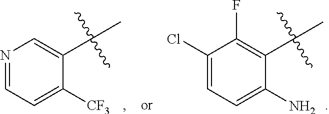 [SMILES: *c1cnccc1C(F)(F)F ; *c1c(N)ccc(Cl)c1F]

In embodiments R¹⁷ is selected from —CH═CH₂,

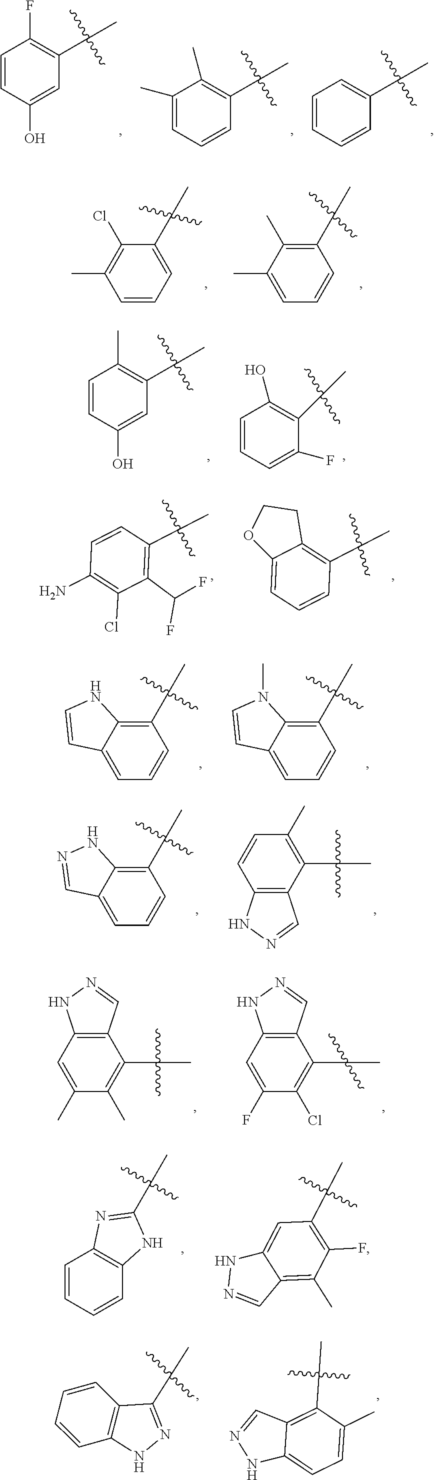

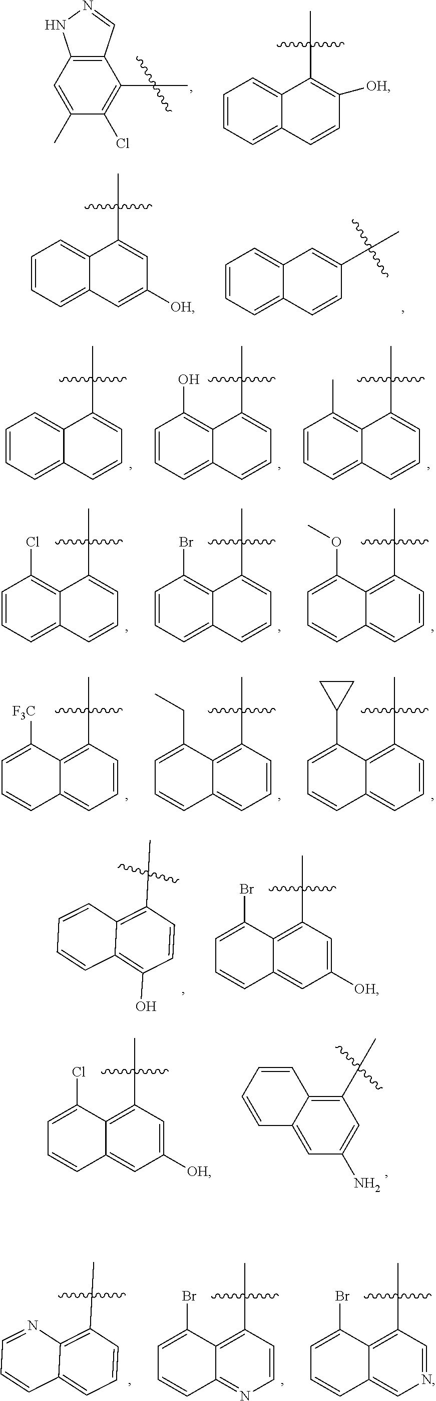

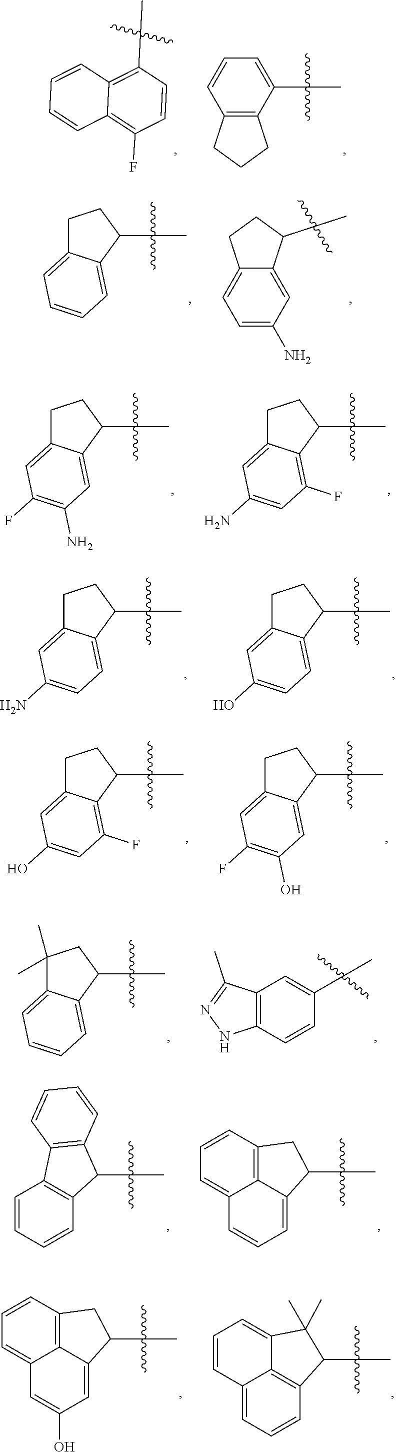

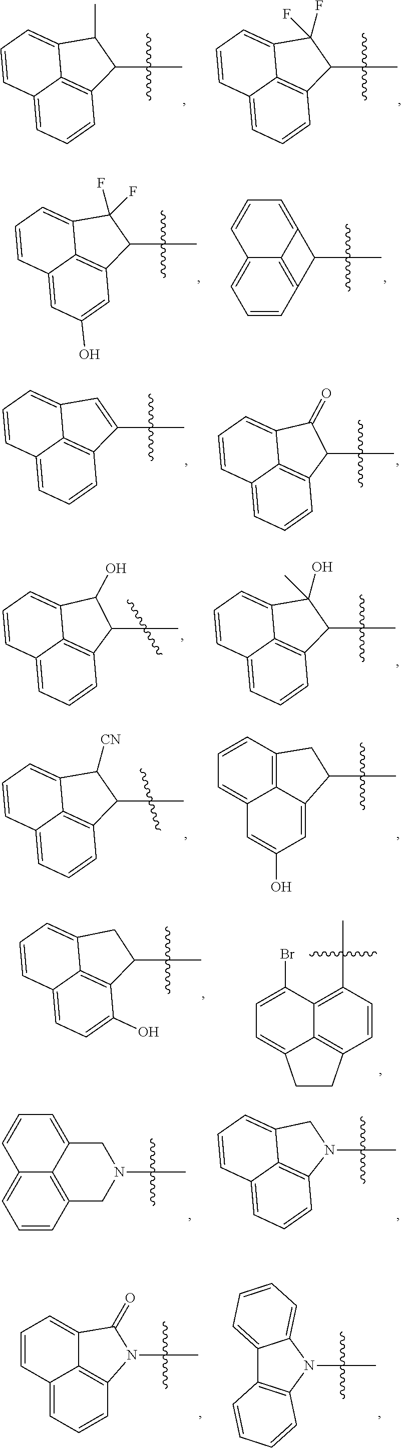

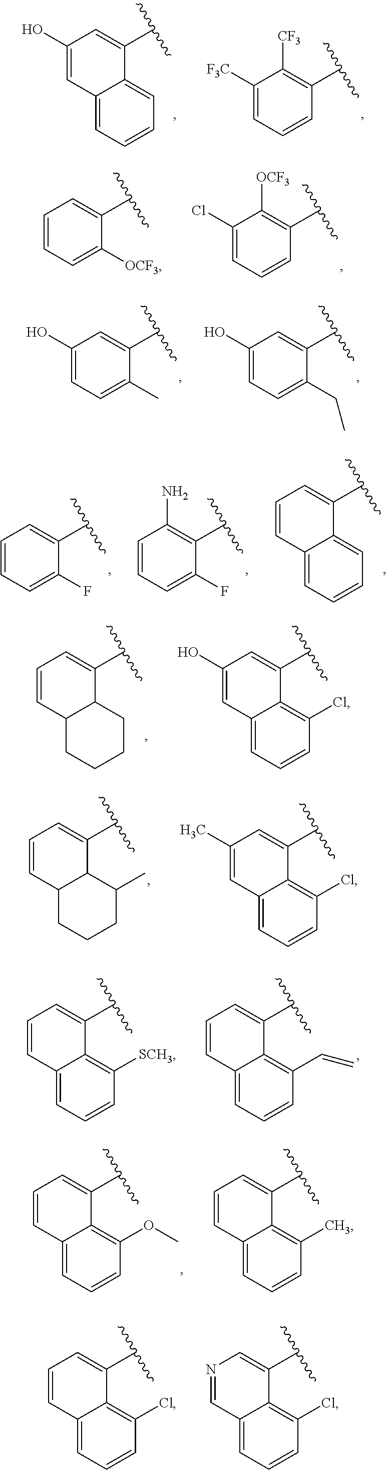

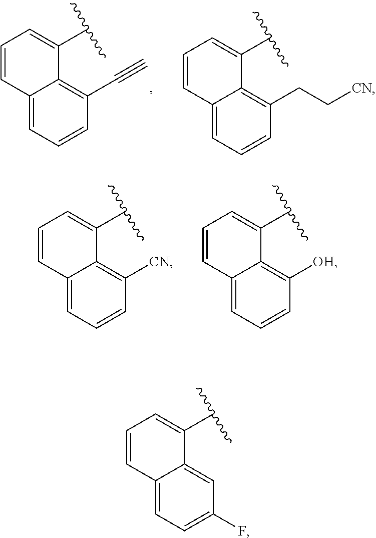

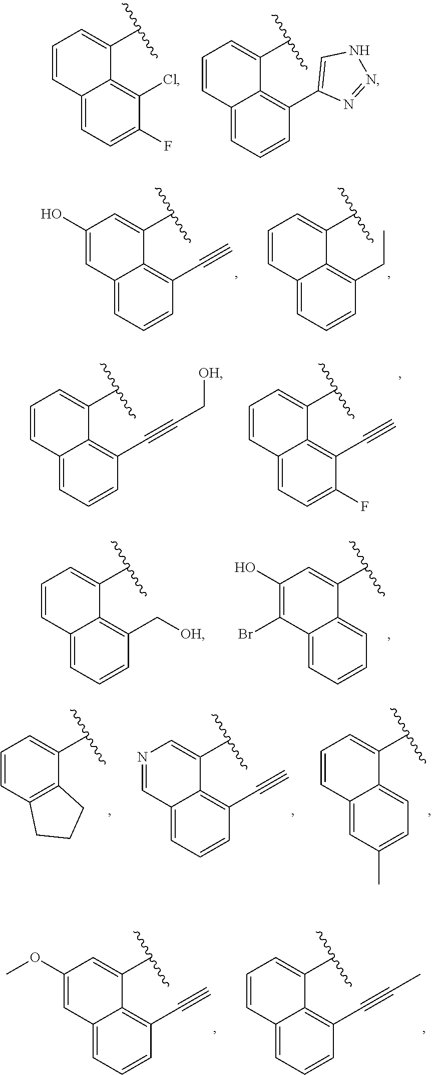

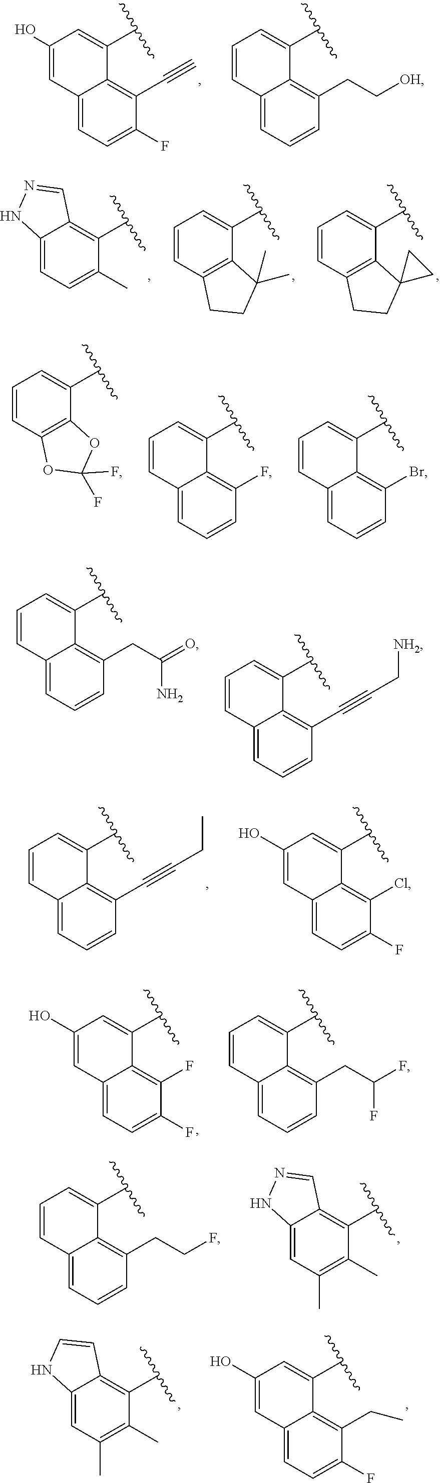

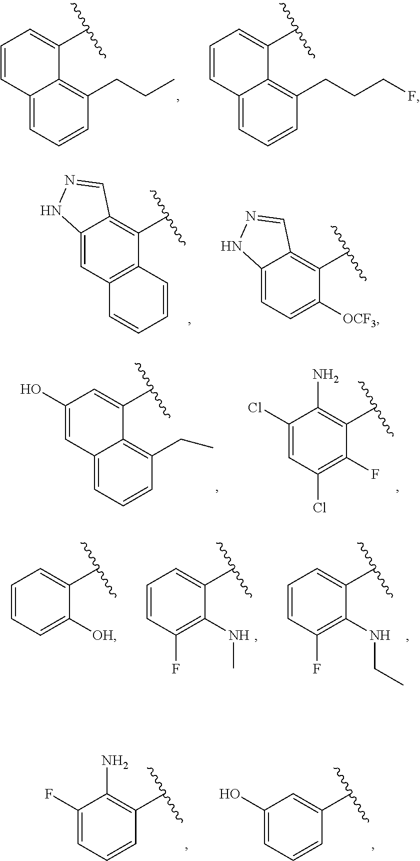

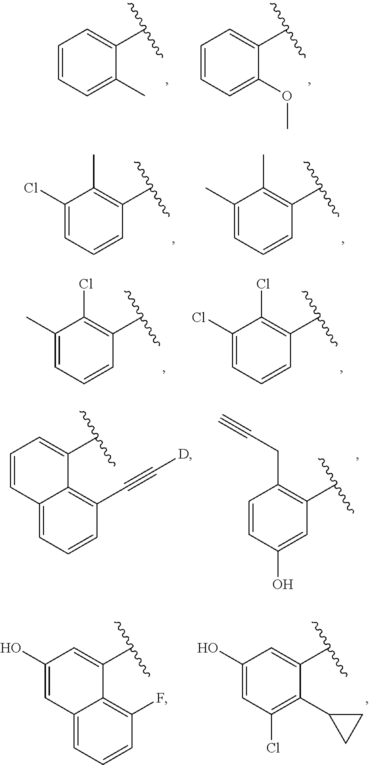

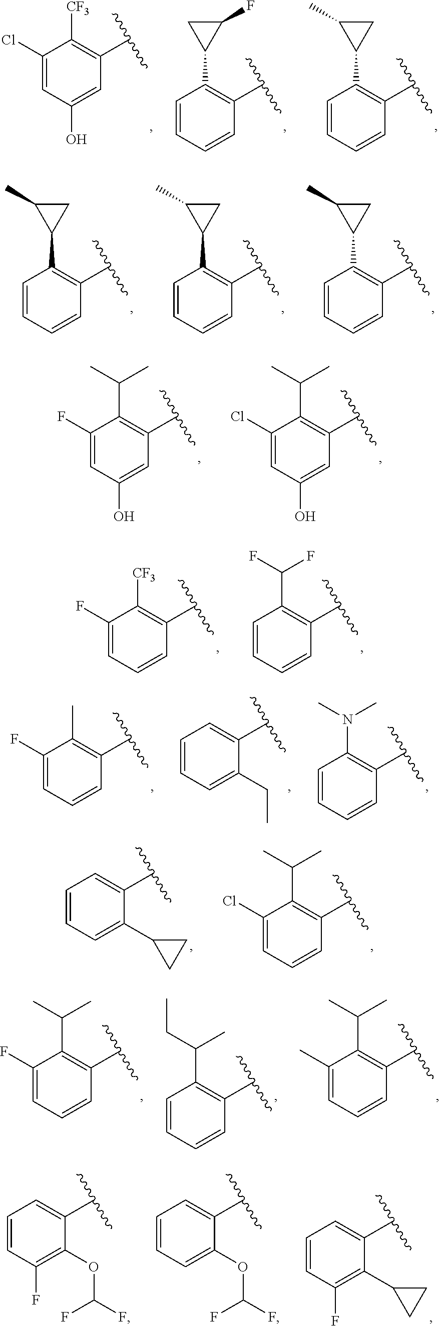

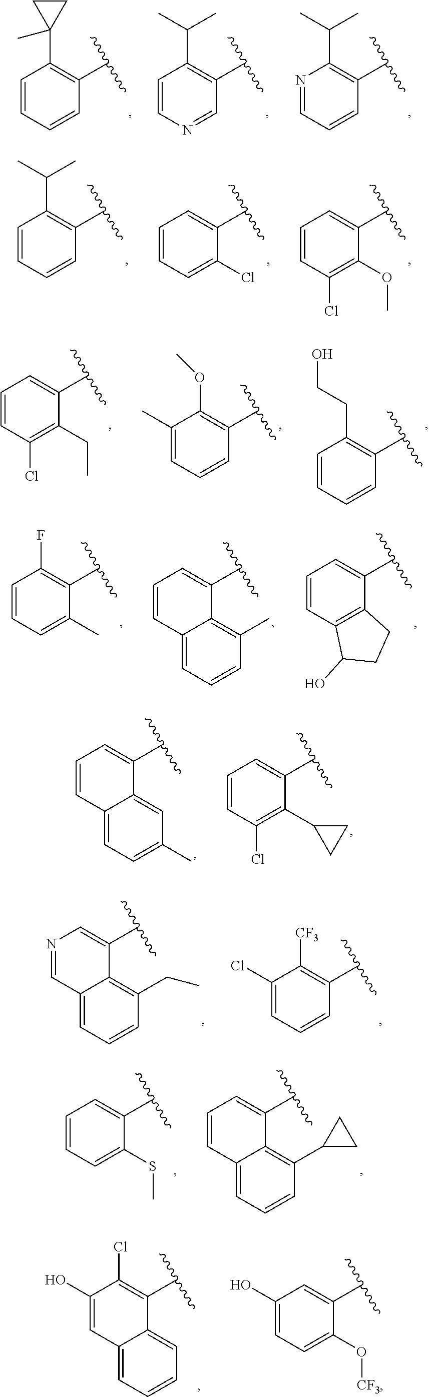

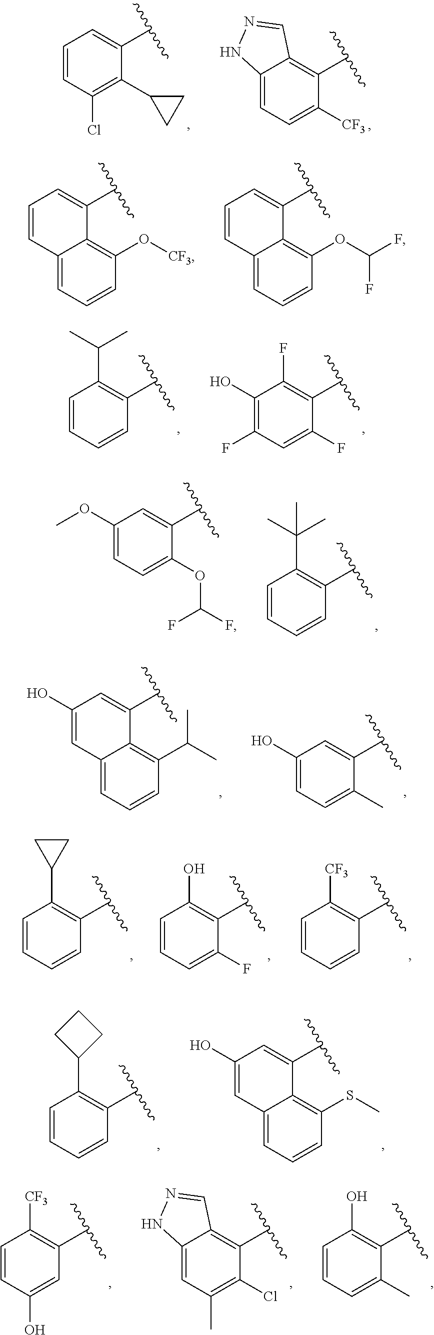

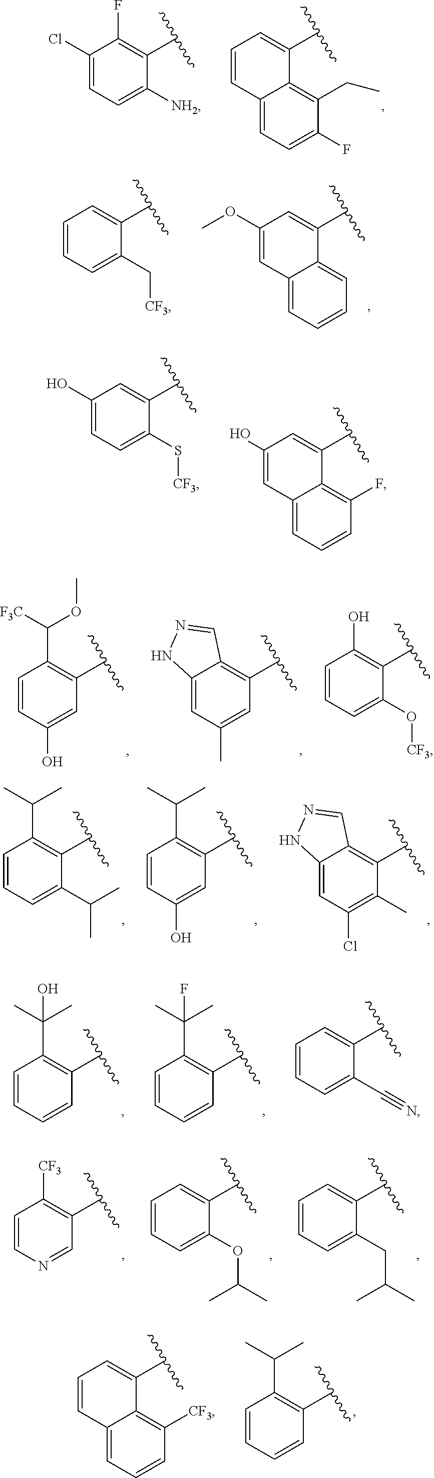

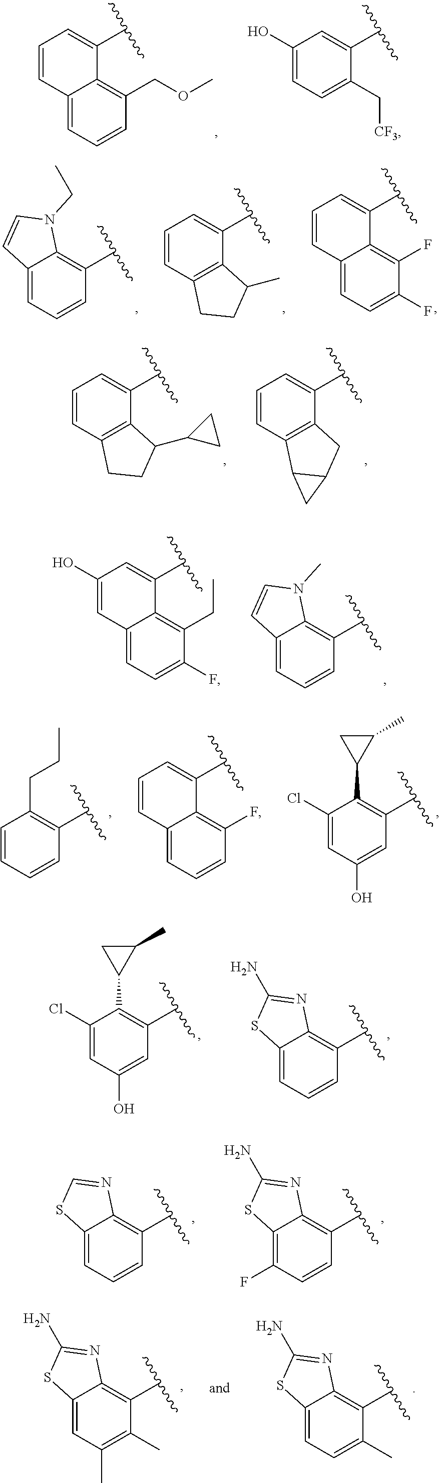

In some embodiments, R¹⁷ is selected from:

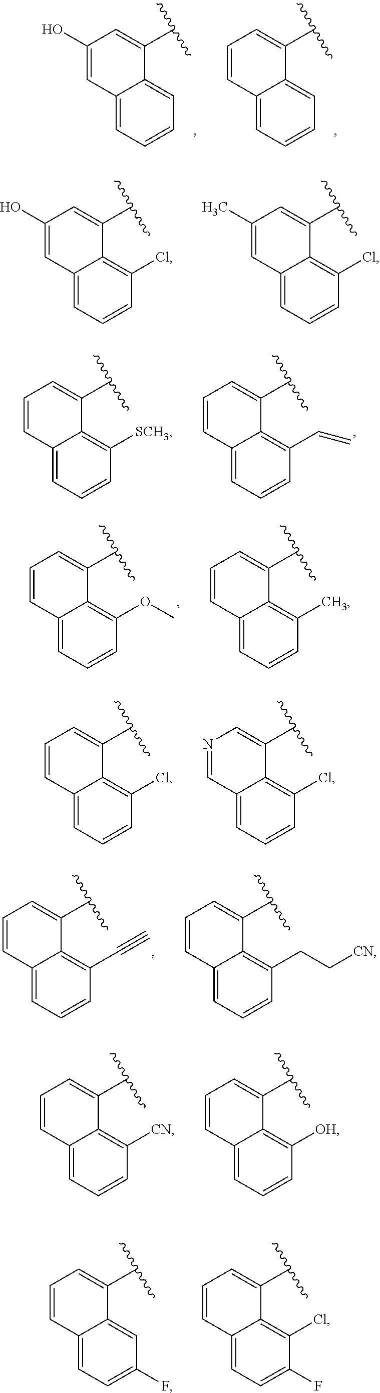

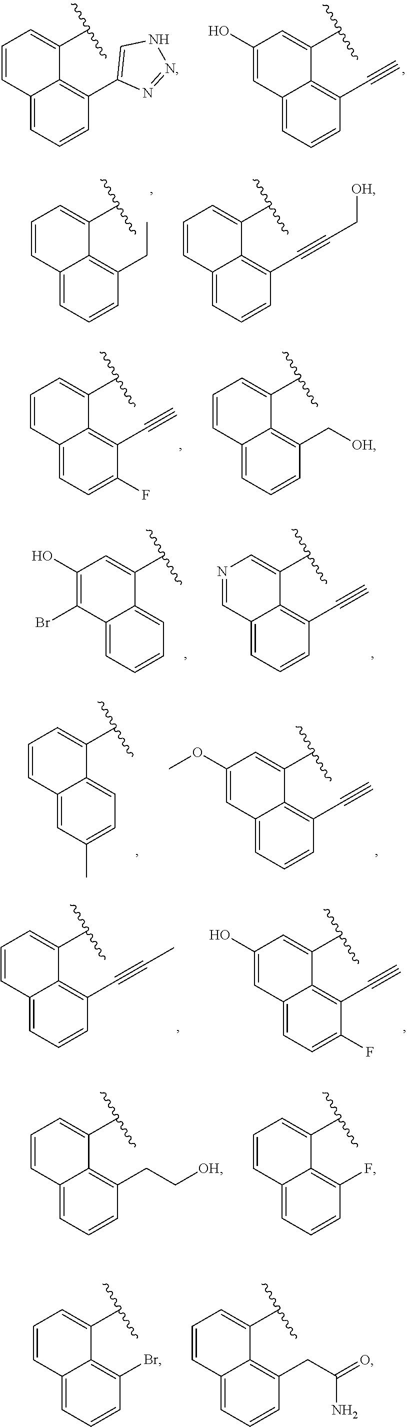

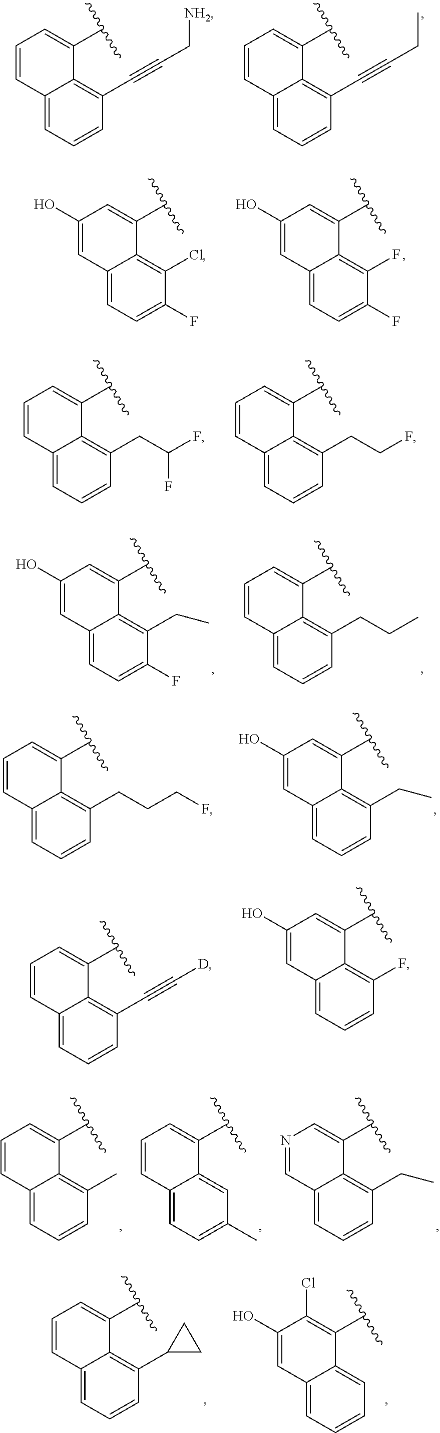

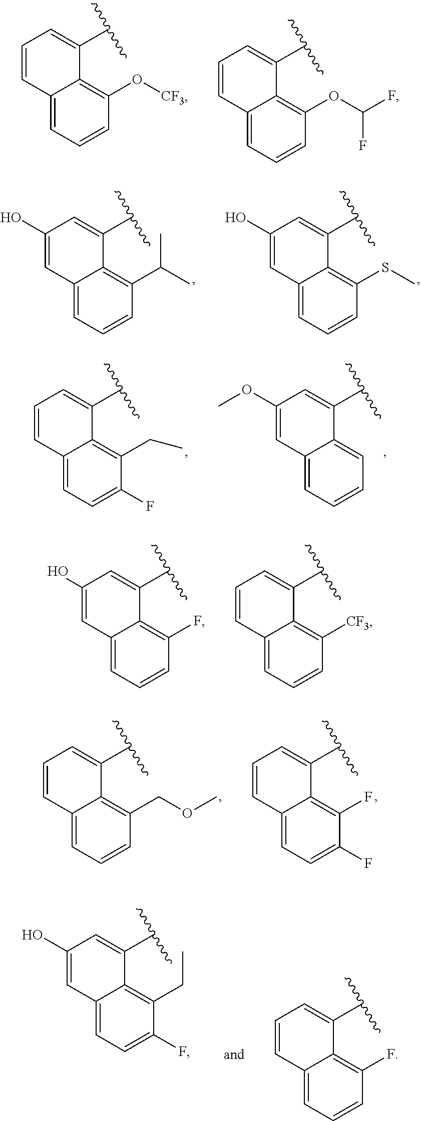

In embodiments of the compound, R¹⁷ is

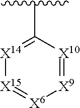

wherein X¹⁴, X¹⁵, X⁶, X′, and X¹⁰ are as described herein. In embodiments of the compound, R¹⁷ is

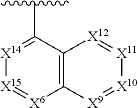

wherein X¹⁴, X¹⁵, X⁶, X′, X¹⁰, X¹¹, and X¹² are as described herein. In embodiments of the compound, R¹⁷ is

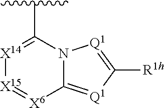

wherein X¹⁴, X¹⁵, X⁶, Q¹, and R^{1a} are as described herein. In embodiments of the compound, R¹⁷ is

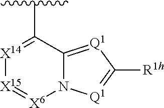

wherein X¹⁴, X¹⁵, X⁶, Q¹, and R¹⁴ are as described herein. In embodiments of the compound, R¹⁷ is

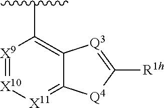

wherein X⁹, X¹⁰, X¹¹, Q³, Q⁴, and R^{1a} are as described herein. In embodiments of the compound, R¹⁷ is

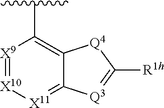

wherein X′, X¹⁰, X¹¹, Q³, Q⁴, and R^{1h} are as described herein. In embodiments of the compound, R¹⁷ is

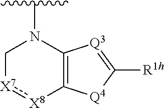

wherein X¹, X⁸, X¹², Q³, Q⁴, and R^{1h} are as described herein. In embodiments of the compound, R¹⁷ is

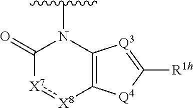

wherein X¹, X⁸, Q³, Q⁴, and R^{1h} are as described herein. In embodiments of the compound, R¹⁷ is

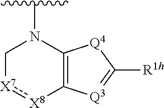

wherein X⁷, X⁸, Q³, Q⁴, and R^{1h} are as described herein. In embodiments of the compound, R¹⁷ is

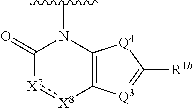

wherein X⁷, X⁸, Q³, Q′, and R^{1h} are as described herein. In embodiments of the compound, R¹⁷ is

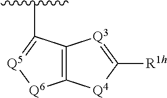

wherein Q³, Q¹, Q⁵, Q⁶, and R^{1h} are as described herein. In embodiments of the compound, R¹⁷ is

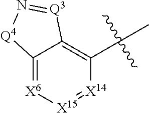

wherein X¹⁴, X¹⁵, X⁶, Q³, and Q⁴ are as described herein. In embodiments of the compound, R¹⁷ is

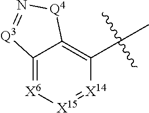

wherein X¹⁴, X¹⁵, X⁶, Q³, and Q⁴ are as described herein. In embodiments of the compound, R¹⁷ is

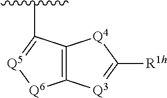

wherein, Q³, Q⁴, Q⁵, Q⁶, and R^{1h} are as described herein. In embodiments of the compound, R¹⁷ is

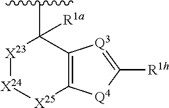

wherein Q³, Q⁴, X²³, X²⁴, X²⁵, R^{1a} are R^{1h} are as described herein.

In embodiments of the compound, R¹⁷ is

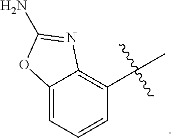 [SMILES: *c1cccc2oc(N)nc12]

In some embodiments of the compound, R¹⁷ is

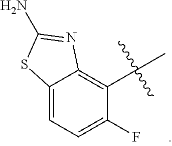 [SMILES: *c1c(F)ccc2sc(N)nc12]

In further embodiments of the compound, R¹⁷ is

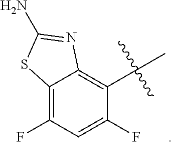 [SMILES: *c1c(F)cc(F)c2sc(N)nc12]

In additional embodiments of the compound, R¹⁷ is

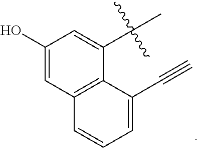 [SMILES: *c1cc(O)cc2cccc(C#C)c12]

In select embodiments of the compound, R¹⁷ is

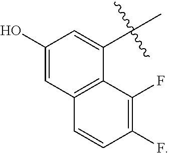 [SMILES: *c1cc(O)cc2ccc(F)c(F)c12]

In embodiments of the compound, R¹⁷ is

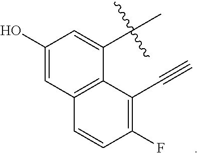 [SMILES: *c1cc(O)cc2ccc(F)c(C#C)c12]

In some embodiments of the compound, R¹⁷ is

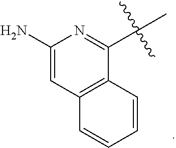 [SMILES: *c1nc(N)cc2ccccc12]

In some embodiments of the compound, R¹⁷ is

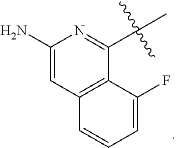 [SMILES: *c1nc(N)cc2cccc(F)c12]

In further embodiments of the compound, R¹⁷ is

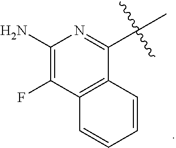 [SMILES: *c1nc(N)c(F)c2ccccc12]

In select embodiments of the compound, R¹⁷ is

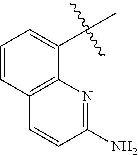 [SMILES: *c1cccc2ccc(N)nc12]

In embodiments of the compound, R¹⁷ is

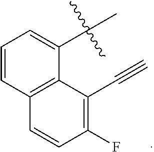 [SMILES: *c1cccc2ccc(F)c(C#C)c12]

In some embodiments of the compound, R¹⁷ is

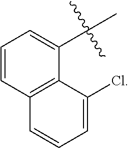 [SMILES: *c1cccc2cccc(Cl)c12]

In additional embodiments of the compound, R¹⁷ is

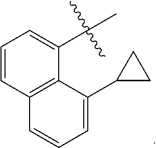 [SMILES: *c1cccc2cccc(C3CC3)c12]

In some embodiments of the compound, R¹⁷ is

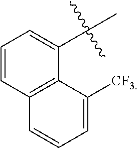 [SMILES: *c1cccc2cccc(C(F)(F)F)c12]

In further embodiments of the compound, R¹⁷ is

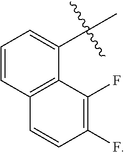 [SMILES: *c1cccc2ccc(F)c(F)c12]

In embodiments of the compound, R¹⁷ is

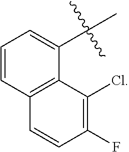 [SMILES: *c1cccc2ccc(F)c(Cl)c12]

In select embodiments of the compound, R¹⁷ is

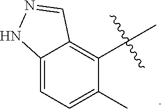 [SMILES: *c1c(C)ccc2[nH]ncc12]

In some embodiments of the compound, R¹⁷ is

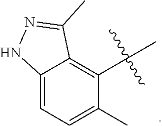 [SMILES: *c1c(C)ccc2[nH]nc(C)c12]

In additional embodiments of the compound, R¹⁷ is

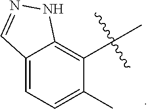 [SMILES: *c1c(C)ccc2cn[nH]c12]

In embodiments of the compound, R¹⁷ is

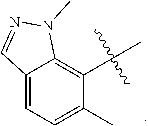 [SMILES: *c1c(C)ccc2cnn(C)c12]

In embodiments of the compound, R¹⁷ is

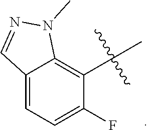 [SMILES: *c1c(F)ccc2cnn(C)c12]

In further embodiments of the compound, R¹⁷ is

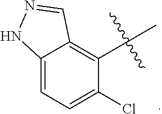 [SMILES: *c1c(Cl)ccc2[nH]ncc12]

In select embodiments of the compound, R¹⁷ is

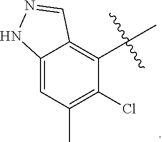 [SMILES: *c1c(Cl)c(C)cc2[nH]ncc12]

In some embodiments of the compound, R¹⁷ is

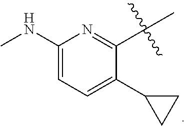 [SMILES: *c1nc(NC)ccc1C1CC1]

In additional embodiments of the compound, R¹⁷ is

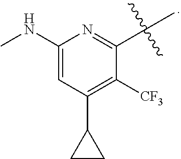 [SMILES: *c1nc(NC)cc(C2CC2)c1C(F)(F)F]

In embodiments of the compound, R¹⁷ is

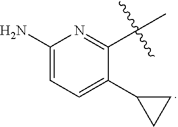 [SMILES: *c1nc(N)ccc1C1CC1]

In some embodiments of the compound, R¹⁷ is

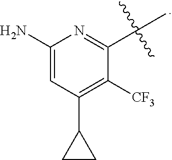 [SMILES: *c1nc(N)cc(C2CC2)c1C(F)(F)F]

In further embodiments of the compound, R¹⁷ is

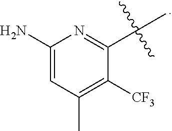 [SMILES: *c1nc(N)cc(C)c1C(F)(F)F]

In select embodiments of the compound, R¹⁷ is

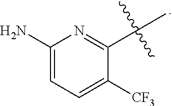 [SMILES: *c1nc(N)ccc1C(F)(F)F]

In some embodiments of the compound, R¹⁷ is

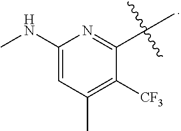 [SMILES: *c1nc(NC)cc(C)c1C(F)(F)F]

In embodiments of the compound, R¹⁷ is

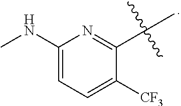 [SMILES: *c1nc(NC)ccc1C(F)(F)F]

In some embodiments of the compound, R¹⁷ is

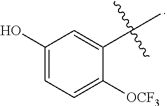 [SMILES: *c1cc(O)ccc1OC(F)(F)F]

In additional embodiments of the compound, R¹⁷ is

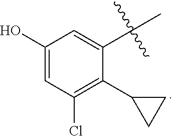 [SMILES: *c1cc(O)cc(Cl)c1C1CC1]

In further embodiments of the compound, R¹⁷ is

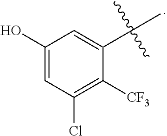 [SMILES: *c1cc(O)cc(Cl)c1C(F)(F)F]

In select embodiments of the compound, R¹⁷ is

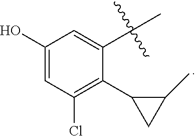 [SMILES: *c1cc(O)cc(Cl)c1C1CC1C]

In embodiments of the compound, R¹⁷ is

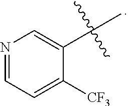 [SMILES: *c1cnccc1C(F)(F)F]

In some embodiments of the compound, R¹⁷ is

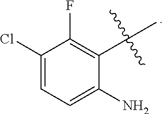 [SMILES: *c1c(N)ccc(Cl)c1F]

In some embodiments of the compound, R¹⁷ is

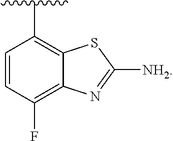 [SMILES: *c1ccc(F)c2nc(N)sc12]

In some embodiments of the compound, R¹⁷ is

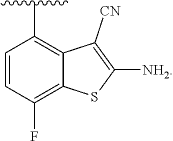 [SMILES: *c1ccc(F)c2sc(N)c(C#N)c12]

In some embodiments of the compound, R¹⁷ is

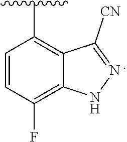 [SMILES: *c1ccc(F)c2[nH]nc(C#N)c12]

In some embodiments of the compound, R¹⁷ is

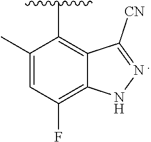 [SMILES: *c1c(C)cc(F)c2[nH]nc(C#N)c12]

In some embodiments of the compound, R¹⁷ is

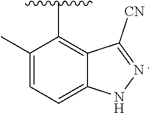 [SMILES: *c1c(C)ccc2[nH]nc(C#N)c12]

In some embodiments of the compound, R¹⁷ is

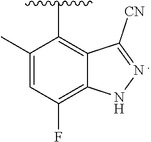 [SMILES: *c1c(C)cc(F)c2[nH]nc(C#N)c12]

In some embodiments of the compound, R¹⁷ is

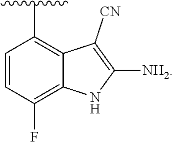 [SMILES: *c1ccc(F)c2[nH]c(N)c(C#N)c12]

In some embodiments of the compound, R¹⁷ is

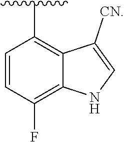 [SMILES: *c1ccc(F)c2[nH]cc(C#N)c12]

In some embodiments of the compound, R¹⁷ is

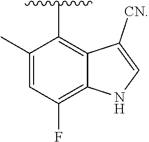 [SMILES: *c1c(C)cc(F)c2[nH]cc(C#N)c12]

In some embodiments of the compound, R¹⁷ is

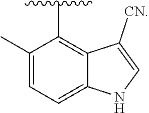 [SMILES: *c1c(C)ccc2[nH]cc(C#N)c12]

In some embodiments of the compound, R¹⁷ is

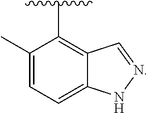 [SMILES: *c1c(C)ccc2[nH]ncc12]

In some embodiments of the compound, R¹⁷ is

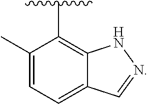 [SMILES: *c1c(C)ccc2cn[nH]c12]

In embodiments of the compound, R¹⁷ is

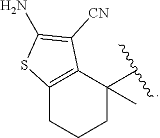 [SMILES: *C1(C)CCCc2sc(N)c(C#N)c21]

In embodiments of the compound, R¹⁷ is

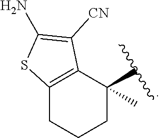 [SMILES: *[C@@]1(C)CCCc2sc(N)c(C#N)c21]

In embodiments of the compound, R¹⁷ is

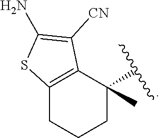 [SMILES: *[C@]1(C)CCCc2sc(N)c(C#N)c21]

In embodiments of the compound, R¹⁷ is

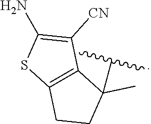 [SMILES: *C1(C)CCc2sc(N)c(C#N)c21]

In embodiments of the compound, R¹⁷ is

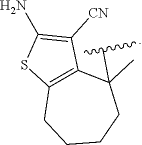 [SMILES: *C1(C)CCCCc2sc(N)c(C#N)c21]

In embodiments of the compound, R¹⁷ is

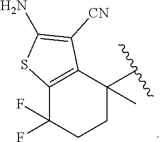 [SMILES: *C1(C)CCC(F)(F)c2sc(N)c(C#N)c21]

In embodiments of the compound, R¹⁷ is

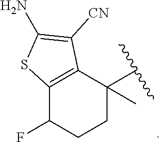 [SMILES: *C1(C)CCC(F)c2sc(N)c(C#N)c21]

In embodiments of the compound, R¹⁷ is

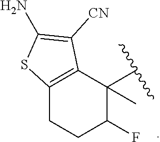 [SMILES: *C1(C)c2c(sc(N)c2C#N)CCC1F]

In embodiments of the compound, R¹⁷ is

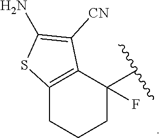 [SMILES: *C1(F)CCCc2sc(N)c(C#N)c21]

In embodiments of the compound, R¹⁷ is

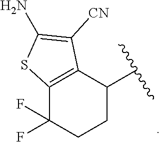 [SMILES: *C1CCC(F)(F)c2sc(N)c(C#N)c21]

In embodiments of the compound, R¹⁷ is

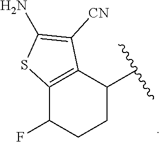 [SMILES: *C1CCC(F)c2sc(N)c(C#N)c21]

In embodiments of the compound, R¹⁷ is

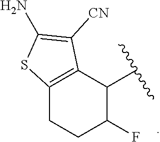 [SMILES: *C1c2c(sc(N)c2C#N)CCC1F]

In embodiments of the compound, R¹⁷ is

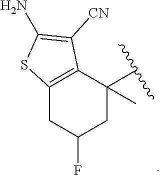 [SMILES: *C1(C)CC(F)Cc2sc(N)c(C#N)c21]

In embodiments of the compound, R¹⁷ is

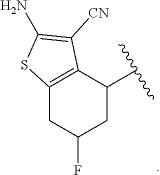 [SMILES: *C1CC(F)Cc2sc(N)c(C#N)c21]

In embodiments of the compound, or a pharmaceutically acceptable salt or solvate thereof, R¹⁹ is:

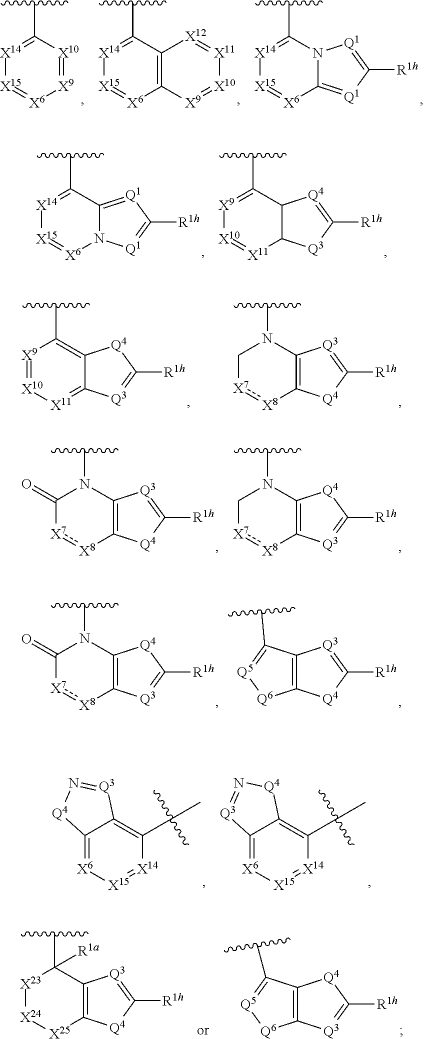

Q¹, Q³, and Q⁵ are independently N or C(R¹⁴);
Q⁴ and Q⁶ are independently O, S, C(R^{1a})(R¹⁸), or N(R^{1c});
X¹⁴, X¹⁵, X⁶, X⁹, X¹⁰, X¹¹, and X¹² are independently selected from C(R¹⁴) or N;
X⁷ and X⁸ are independently selected from C(R^{1a}), C(R^{1a})(R^{1b}), N, or N(R^{1c});
X²³ is selected from a bond, C(R^{1a})(R^{1b}), C(R^{1a})(R^{1b})C(R^{1a})(R^{1b}), C(R^{1a})(R^{1b})N(R^{1c}), and N(R^{1c});
X²⁴ and X²⁵ are independently selected from a bond, C(R^{1a})(R^{1b}), and N(R^{1c});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments, R^{1h} is selected from hydrogen, C₁₋₆alkyl, C₁₋₆haloalkyl, C₂₋₆alkenyl, C₂₋₆alkynyl, —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —C(O)R¹⁵, —OC(O)R¹⁵, —C(O)N(R¹²)(R¹³), —C(O)C(O)N(R¹²)(R¹³), —N(R¹⁴)C(O)R¹⁵, —CH₂C(O)N(R¹²)(R¹³), and —CH₂N(R¹⁴)C(O)R¹⁵, wherein C₁₋₆alkyl, C₂₋₆alkenyl, and C₂₋₆alkynyl are optionally substituted with one, two, or three R²⁰ⁱ.

In embodiments, R^{1h} is selected from hydrogen and —N(R¹²)(R¹³). In embodiments, R^{1h} is hydrogen. In embodiments, R^{1h} is —N(R¹²)(R¹³). In embodiments, R^{1h} is —NH₂.

In embodiments, R^{1a} is independently hydrogen. In embodiments, R^{1a} is independently halogen. In embodiments, R^{1a} is independently —CN. In embodiments, R^{1a} is independently C₁₋₆alkyl optionally substituted with one, two, or three R²⁰ⁱ. In embodiments, R^{1a} is independently C₁₋₆haloalkyl. In embodiments, R^{1a} is independently C₂₋₆alkenyl optionally substituted with one, two, or three R²⁰ⁱ. In embodiments, R^{1a} is independently C₂₋₆alkynyl optionally substituted with one, two, or three R²⁰ⁱ. In embodiments, R^{1a} is independently C₃₋₁₀cycloalkyl optionally substituted with one, two, or three R²⁰ⁱ. In embodiments, R^{1a} is independently C₃₋₆cycloalkyl optionally substituted with one, two, or three R²⁰ⁱ. In embodiments, R^{1a} is independently C₂₋₉heterocycloalkyl optionally substituted with one, two, or three R²⁰ⁱ. In embodiments, R^{1a} is independently C₆₋₁₀aryl optionally substituted with one, two, or three R²⁰ⁱ. In embodiments, R^{1a} is independently C₁₋₉heteroaryl optionally substituted with one, two, or three R²⁰ⁱ. In embodiments, R^{1a} is independently —OR¹². In embodiments, R^{1a} is independently —SR¹². In embodiments, R^{1a} is independently —N(R¹²)(R¹³). In embodiments, R^{1a} is independently —C(O)OR¹². In embodiments, R^{1a} is independently —OC(O)N(R¹²)(R¹³). In embodiments, R^{1a} is independently —N(R¹⁴)C(O)N(R¹²)(R¹³). In embodiments, R^{1a} is independently —N(R¹⁴)C(O)OR¹⁵. In embodiments, R^{1a} is independently —N(R¹⁴)S(O)₂R¹⁵. In embodiments, R^{1a} is independently —C(O)R¹⁵. In embodiments, R^{1a} is independently —S(O)R¹⁵. In embodiments, R^{1a} is independently —OC(O)R¹⁵. In embodiments, R^{1a} is independently —C(O)N(R¹²)(R¹³). In embodiments, R^{1a} is independently —C(O)C(O)N(R¹²)(R¹³). In embodiments, R^{1a} is independently —N(R¹⁴)C(O)R¹⁵. In embodiments, R^{1a} is independently —S(O)₂R¹⁵. In embodiments, R^{1a} is independently —S(O)₂N(R¹²)(R¹³). In embodiments, R^{1a} is independently —S(═O)(═NH)N(R¹²)(R¹³). In embodiments, R^{1a} is independently —CH₂C(O)N(R¹²)(R¹³). In embodiments, R^{1a} is independently —CH₂N(R¹⁴)C(O)R¹⁵. In embodiments, R^{1a} is independently —CH₂S(O)₂R¹⁵. In embodiments, R^{1a} is independently —CH₂S(O)₂N(R¹²)(R¹³). In embodiments, R^{1a} is independently —OH. In embodiments, R^{1a} is independently —SH. In embodiments, R^{1a} is independently —NH₂. In embodiments, R^{1a} is independently —C(O)OH. In embodiments, R^{1a} is independently —OC(O)NH₂. In embodiments, R^{1a} is independently —N(H)C(O)NH₂. In embodiments, R^{1a} is independently —N(H)C(O)OH. In embodiments, R^{1a} is independently —N(H)S(O)₂CH₃. In embodiments, R^{1a} is independently —C(O)CH₃. In embodiments, R^{1a} is independently —S(O)CH₃. In embodiments, R^{1a} is independently —OC(O)CH₃. In embodiments, R^{1a} is independently —C(O)NH₂. In embodiments, R^{1a} is independently —C(O)C(O)NH₂. In embodiments, R^{1a} is independently —N(H)C(O)CH₃. In embodiments, R^{1a} is independently —S(O)₂CH₃. In embodiments, R^{1a} is independently —S(O)₂NH₂. In embodiments, R^{1a} is independently —S(—O)(═NH)NH₂. In embodiments, R^{1a} is independently —CH₂C(O)NH₂. In embodiments, R^{1a} is independently —CH₂N(H)C(O)CH₃. In embodiments, R^{1a} is independently —CH₂S(O)₂CH₃. In embodiments, R^{1a} is independently —CH₂S(O)₂NH₂.

In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, —CN, C₁₋₆alkyl, and C₁₋₆haloalkyl, wherein C₁₋₆alkyl is optionally substituted with one, two, or three R²⁰ⁱ. In embodiments, R^{1d} is independently hydrogen. In embodiments, R^{1d} is independently —CN.

In some embodiments, R¹⁹ is selected from:

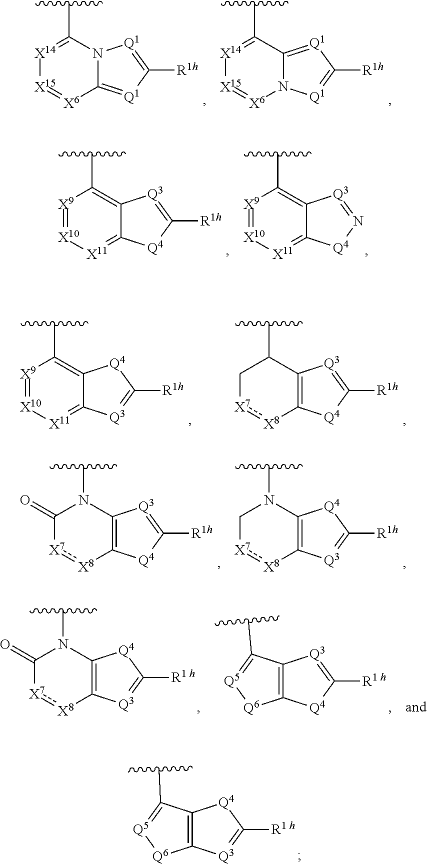

Q¹, Q³, and Q⁵ are independently N or C(R^{1d});
Q⁴ and Q⁶ are independently O, S, C(R^{1a})(R^{1b}), or N(R^{1c});
X¹⁴, X¹⁵, X⁶, X⁹, X¹⁰, and X¹¹ are independently selected from C(R^{1a}) or N;
X⁷ and X⁸ are independently selected from C(R^{1a}), C(R^{1a})(R^{1b}), N, or N(R^{1c});
each R^{1a}, R^{1b}, R^{1d}, R^{1f}, R^{1g}, and R^{1h} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ, or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, and each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

In embodiments, R^{1f} is independently hydrogen. In embodiments, R^{1f} is independently halogen. In embodiments, Rif is independently —CN. In embodiments, R^{1f} is independently C₁₋₆alkyl optionally substituted with one, two, or three R²⁰ⁱ. In embodiments, R^{1f} is independently C₁₋₆haloalkyl. In embodiments, R^{1f} is independently C₂₋₆alkenyl optionally substituted with one, two, or three R²⁰ⁱ. In embodiments, R^{1f} is independently C₂₋₆alkynyl optionally substituted with one, two, or three R²⁰ⁱ. In embodiments, R^{1f} is independently C₃₋₁₀cycloalkyl optionally substituted with one, two, or three R²⁰ⁱ. In embodiments, Rif is independently C₃₋₆cycloalkyl optionally substituted with one, two, or three R²⁰ⁱ. In embodiments, R^{1f} is independently C₂₋₉heterocycloalkyl optionally substituted with one, two, or three R²⁰ⁱ. In embodiments, R^{1f} is independently C₆₋₁₀aryl optionally substituted with one, two, or three R²⁰ⁱ. In embodiments, R^{1f} is independently C₁₋₉heteroaryl optionally substituted with one, two, or three R²⁰ⁱ. In embodiments, R^{1f} is independently —OR¹². In embodiments, R^{1f} is independently —SR¹². In embodiments, Rif is independently —N(R¹²)(R¹³). In embodiments, R^{1f} is independently —C(O)OR¹². In embodiments, R^{1f} is independently —OC(O)N(R¹²)(R¹³). In embodiments, R^{1f} is independently —N(R¹⁴)C(O)N(R¹²)(R¹³). In embodiments, R^{1f} is independently —N(R¹⁴)C(O)OR¹⁵. In embodiments, R^{1f} is independently —N(R¹⁴)S(O)₂R¹⁵. In embodiments, R^{1f} is independently —C(O)R¹⁵. In embodiments, R^{1f} is independently —S(O)R¹⁵. In embodiments, R^{1f} is independently —OC(O)R¹⁵. In embodiments, R^{1f} is independently —C(O)N(R¹²)(R¹³). In embodiments, R^{1f} is independently —C(O)C(O)N(R¹²)(R¹³). In embodiments, R^{1f} is independently —N(R¹⁴)C(O)R¹⁵. In embodiments, R^{1f} is independently —S(O)₂R¹⁵. In embodiments, R^{1f} is independently —S(O)₂N(R¹²)(R¹³). In embodiments, R^{1f} is independently —S(═OX(═NH)N(R¹²)(R¹³). In embodiments, R^{1f} is independently —CH₂C(O)N(R¹²)(R¹³). In embodiments, R^{1f} is independently —CH₂N(R¹⁴)C(O)R¹⁵. In embodiments, R^{1f} is independently —CH₂S(O)₂R¹⁵. In embodiments, R^{1f} is independently —CH₂S(O)₂N(R¹²)(R¹³). In embodiments, R^{1f} is independently —OH. In embodiments, R^{1f} is independently —SH. In embodiments, R^{1f} is independently —NH₂. In embodiments, R^{1f} is independently —C(O)OH. In embodiments, R^{1f} is independently —OC(O)NH₂. In embodiments, R^{1f} is independently —N(H)C(O)NH₂. In embodiments, R^{1f} is independently —N(H)C(O)OH. In embodiments, R^{1f} is independently —N(H)S(O)₂CH₃. In embodiments, R^{1f} is independently —C(O)CH₃. In embodiments, R^{1f} is independently —S(O)CH₃. In embodiments, R^{1f} is independently —OC(O)CH₃. In embodiments, R^{1f} is independently —C(O)NH₂. In embodiments, R^{1f} is independently —C(O)C(O)NH₂. In embodiments, R^{1f} is independently —N(H)C(O)CH₃. In embodiments, Rif is independently —S(O)₂CH₃. In embodiments, R^{1f} is independently —S(O)₂NH₂. In embodiments, Rf is independently —S(═O)(═NH)NH₂. In embodiments, R^{1f} is independently —CH₂C(O)NH₂. In embodiments, R^{1f} is independently —CH₂N(H)C(O)CH₃.

In embodiments, R^{1f} is independently —CH₂S(O)₂CH₃. In embodiments, R^{1f} is independently —CH₂S(O)₂NH₂. In embodiments, R^{1a} is independently hydrogen. In embodiments, R^{1a} is independently halogen. In embodiments, R^{1a} is independently —CN. In embodiments, R^{1a} is independently C₁₋₆alkyl optionally substituted with one, two, or three R²⁰ⁱ. In embodiments, R^{1a} is independently C₁₋₆haloalkyl. In embodiments, R^{1a} is independently C₂₋₆alkenyl optionally substituted with one, two, or three R²⁰ⁱ. In embodiments, R^{1a} is independently C₂₋₆alkynyl optionally substituted with one, two, or three R²⁰ⁱ. In embodiments, R^{1a} is independently C₃₋₁₀cycloalkyl optionally substituted with one, two, or three R²⁰ⁱ. In embodiments, R^{1a} is independently C₃₋₆cycloalkyl optionally substituted with one, two, or three R²⁰ⁱ. In embodiments, R^{1a} is independently C₂₋₉heterocycloalkyl optionally substituted with one, two, or three R²⁰ⁱ. In embodiments, R^{1a} is independently C₆₋₁₀aryl optionally substituted with one, two, or three R²⁰ⁱ. In embodiments, R^{1a} is independently C₁₋₉heteroaryl optionally substituted with one, two, or three R²⁰ⁱ. In embodiments, R^{1a} is independently —OR¹². In embodiments, R^{1a} is independently —SR¹². In embodiments, R^{1a} is independently —N(R¹²)(R¹³). In embodiments, R^{1a} is independently —C(O)OR¹². In embodiments, R^{1a} is independently —OC(O)N(R¹²)(R¹³). In embodiments, R^{1a} is independently —N(R¹⁴)C(O)N(R¹²)(R¹³). In embodiments, R^{1a} is independently —N(R¹⁴)C(O)OR¹⁵. In embodiments, R^{1a} is independently —N(R¹⁴)S(O)₂R¹⁵. In embodiments, R^{1a} is independently —C(O)R¹⁵. In embodiments, R^{1a} is independently —S(O)R¹⁵. In embodiments, R^{1a} is independently —OC(O)R¹⁵. In embodiments, R^{1a} is independently —C(O)N(R¹²)(R¹³). In embodiments, R^{1a} is independently —C(O)C(O)N(R¹²)(R¹³). In embodiments, R^{1a} is independently —N(R¹⁴)C(O)R¹⁵. In embodiments, R^{1a} is independently —S(O)₂R¹⁵. In embodiments, R^{1a} is independently —S(O)₂N(R¹²)(R¹³). In embodiments, R^{1a} is independently —S(═O)(═NH)N(R¹²)(R¹³). In embodiments, R^{1a} is independently —CH₂C(O)N(R¹²)(R¹³). In embodiments, R^{1a} is independently —CH₂N(R¹⁴)C(O)R¹⁵. In embodiments, R^{1a} is independently —CH₂S(O)₂R¹⁵. In embodiments, R^{1a} is independently —CH₂S(O)₂N(R¹²)(R¹³). In embodiments, R^{1a} is independently —OH. In embodiments, R^{1a} is independently —SH. In embodiments, R^{1a} is independently —NH₂. In embodiments, R^{1a} is independently —C(O)OH. In embodiments, R^{1a} is independently —OC(O)NH₂. In embodiments, R^{1a} is independently —N(H)C(O)NH₂. In embodiments, R^{1a} is independently —N(H)C(O)OH. In embodiments, R^{1a} is independently —N(H)S(O)₂CH₃. In embodiments, R^{1a} is independently —C(O)CH₃. In embodiments, R^{1a} is independently —S(O)CH₃. In embodiments, R^{1a} is independently —OC(O)CH₃. In embodiments, R^{1a} is independently —C(O)NH₂. In embodiments, R^{1a} is independently —C(O)C(O)NH₂. In embodiments, R^{1a} is independently —N(H)C(O)CH₃. In embodiments, R^{1a} is independently —S(O)₂CH₃. In embodiments, R^{1a} is independently —S(O)₂NH₂. In embodiments, R^{1a} is independently —S(═O)(═NH)NH₂. In embodiments, R^{1a} is independently —CH₂C(O)NH₂. In embodiments, R^{1a} is independently —CH₂N(H)C(O)CH₃. In embodiments, R^{1a} is independently —CH₂S(O)₂CH₃. In embodiments, R^{1a} is independently —CH₂S(O)₂NH₂.

In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}. In embodiments, R^{1d} is independently selected from hydrogen, —CN, C₁₋₆alkyl, and C₁₋₆haloalkyl, wherein C₁₋₆alkyl is optionally substituted with one, two, or three R²⁰ⁱ. In embodiments, R^{1d} is independently hydrogen. In embodiments, R^{1d} is independently —CN.

In embodiments, R¹⁹ is

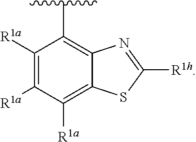

In embodiments, R¹⁹ is

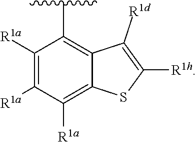

In embodiments, R¹⁹ is

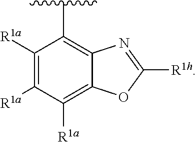

In embodiments, R¹⁹ is

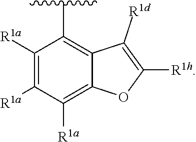

In embodiments, R¹⁹ is

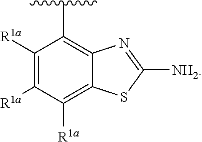

In embodiments, R¹⁹ is

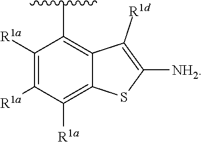

In embodiments, R¹⁹ is

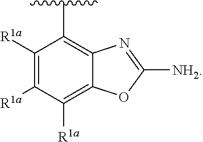

In embodiments, R¹⁹ is

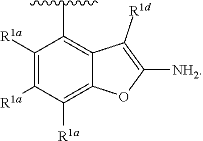

In embodiments, R¹⁹ is

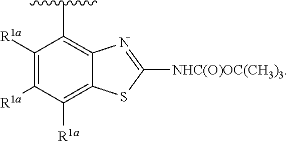

In embodiments, R¹⁹ is

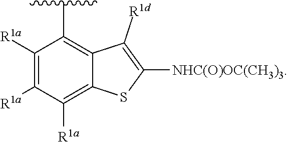

In embodiments, R¹⁹ is

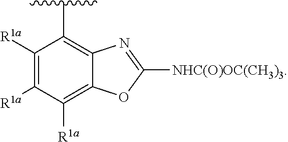

In embodiments, R¹⁹ is

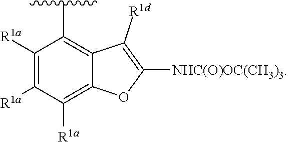

In embodiments, R¹⁹ is

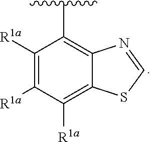

In embodiments, R¹⁹ is

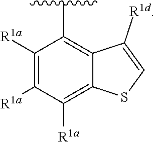

In embodiments, R¹⁹ is

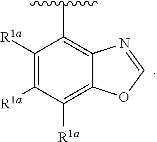

In embodiments, R¹⁹ is

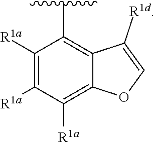

In embodiments, R¹⁹ is

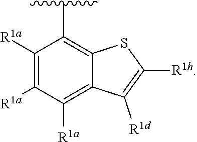

In embodiments, R¹⁹ is

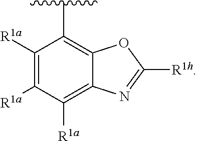

In embodiments, R¹⁹ is

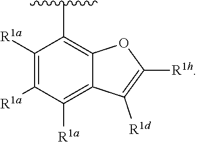

In embodiments, R¹⁹ is

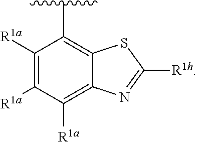

In embodiments, R¹⁹ is

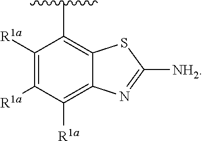

In embodiments, R¹⁹ is

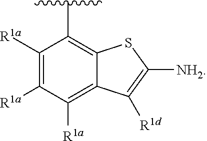

In embodiments, R¹⁹ is

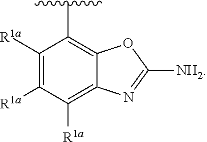

In embodiments, R¹⁹ is

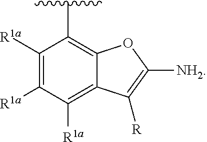

In embodiments, R¹⁹ is

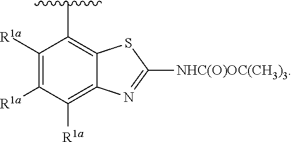

In embodiments, R¹⁹ is

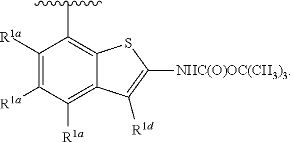

In embodiments, R¹⁹ is

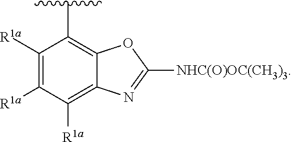

In embodiments, R¹⁹ is

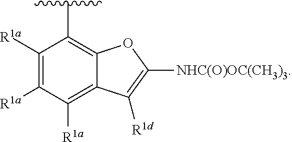

In embodiments, R¹⁹ is

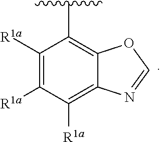

In embodiments, R¹⁹ is

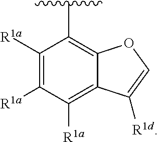

In embodiments, R¹⁹ is

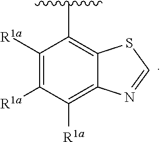

In embodiments, R¹⁹ is

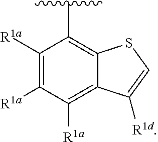

In some embodiments, R¹⁹ is

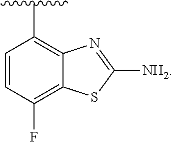 [SMILES: *c1ccc(F)c2sc(N)nc12]

In some embodiments, R¹⁹ is

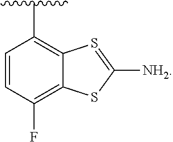

In some embodiments, R¹⁹ is

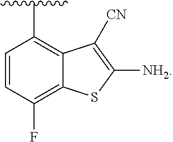 [SMILES: *c1ccc(F)c2sc(N)c(C#N)c12]

In some embodiments, R¹⁹ is

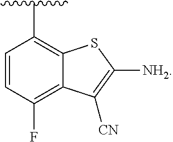 [SMILES: *c1ccc(F)c2c(C#N)c(N)sc12]

In some embodiments, R¹⁹ is

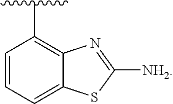 [SMILES: *c1cccc2sc(N)nc12]

In some embodiments, R¹⁹ is

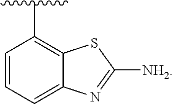 [SMILES: *c1cccc2nc(N)sc12]

In some embodiments, R¹⁹ is

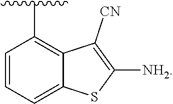 [SMILES: *c1cccc2sc(N)c(C#N)c12]

In some embodiments, R¹⁹ is

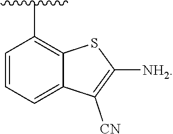 [SMILES: *c1cccc2c(C#N)c(N)sc12]

In some embodiments, R¹⁹ is

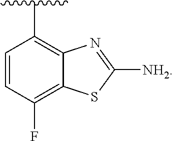 [SMILES: *c1ccc(F)c2sc(N)nc12]

In some embodiments, R¹⁹ is

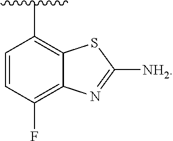 [SMILES: *c1ccc(F)c2nc(N)sc12]

In some embodiments, R¹⁹ is

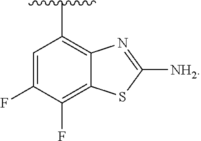 [SMILES: *c1cc(F)c(F)c2sc(N)nc12]

In some embodiments, R¹⁹ is

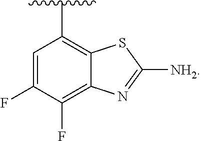 [SMILES: *c1cc(F)c(F)c2nc(N)sc12]

In some embodiments, R¹⁹ is

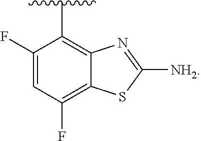 [SMILES: *c1c(F)cc(F)c2sc(N)nc12]

In some embodiments, R¹⁹ is

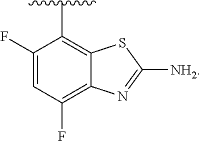 [SMILES: *c1c(F)cc(F)c2nc(N)sc12]

In some embodiments, R¹⁹ is

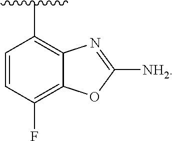 [SMILES: *c1ccc(F)c2oc(N)nc12]

In some embodiments, R¹⁹ is

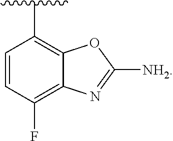 [SMILES: *c1ccc(F)c2nc(N)oc12]

In some embodiments, R¹⁹ is

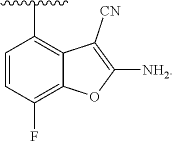 [SMILES: *c1ccc(F)c2oc(N)c(C#N)c12]

In some embodiments, R¹⁹ is

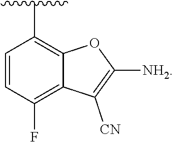 [SMILES: *c1ccc(F)c2c(C#N)c(N)oc12]

In some embodiments, R¹⁹ is

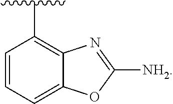 [SMILES: *c1cccc2oc(N)nc12]

In some embodiments, R¹⁹ is

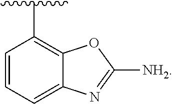 [SMILES: *c1cccc2nc(N)oc12]

In some embodiments, R¹⁹ is

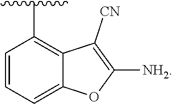 [SMILES: *c1cccc2oc(N)c(C#N)c12]

In some embodiments, R¹⁹ is

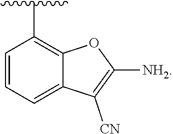 [SMILES: *c1cccc2c(C#N)c(N)oc12]

In some embodiments, R¹⁹ is

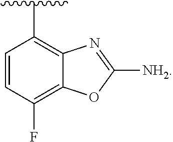 [SMILES: *c1ccc(F)c2oc(N)nc12]

In some embodiments, R¹⁹ is

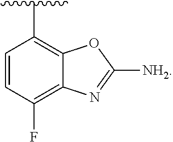 [SMILES: *c1ccc(F)c2nc(N)oc12]

In some embodiments, R¹⁹ is

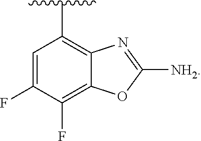 [SMILES: *c1cc(F)c(F)c2oc(N)nc12]

In some embodiments, R¹⁹ is

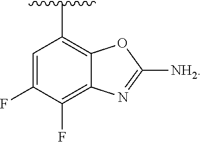 [SMILES: *c1cc(F)c(F)c2nc(N)oc12]

In some embodiments, R¹⁹ is

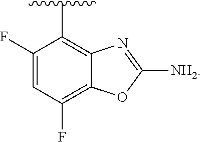 [SMILES: *c1c(F)cc(F)c2oc(N)nc12]

In some embodiments, R¹⁹ is

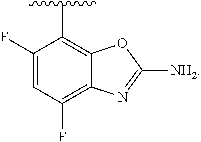 [SMILES: *c1c(F)cc(F)c2nc(N)oc12]

In some embodiments, R¹⁹ is selected from:

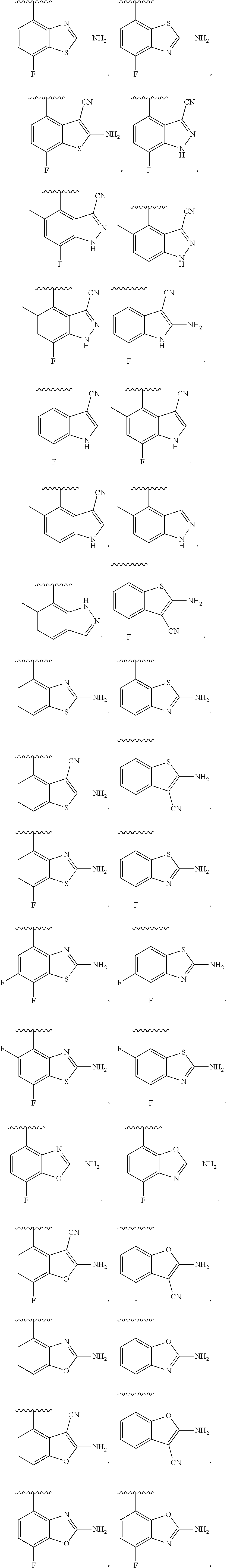

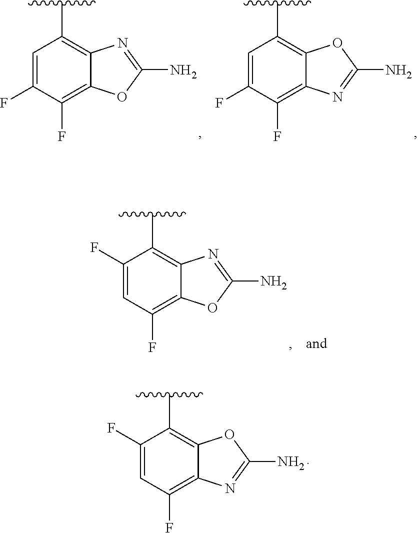 [SMILES: *c1cc(F)c(F)c2oc(N)nc12 ; *c1cc(F)c(F)c2nc(N)oc12 ; *c1c(F)cc(F)c2oc(N)nc12 ; *c1c(F)cc(F)c2nc(N)oc12]

In some embodiments of the compound, or a pharmaceutically acceptable salt or solvate thereof, R¹⁹ is:

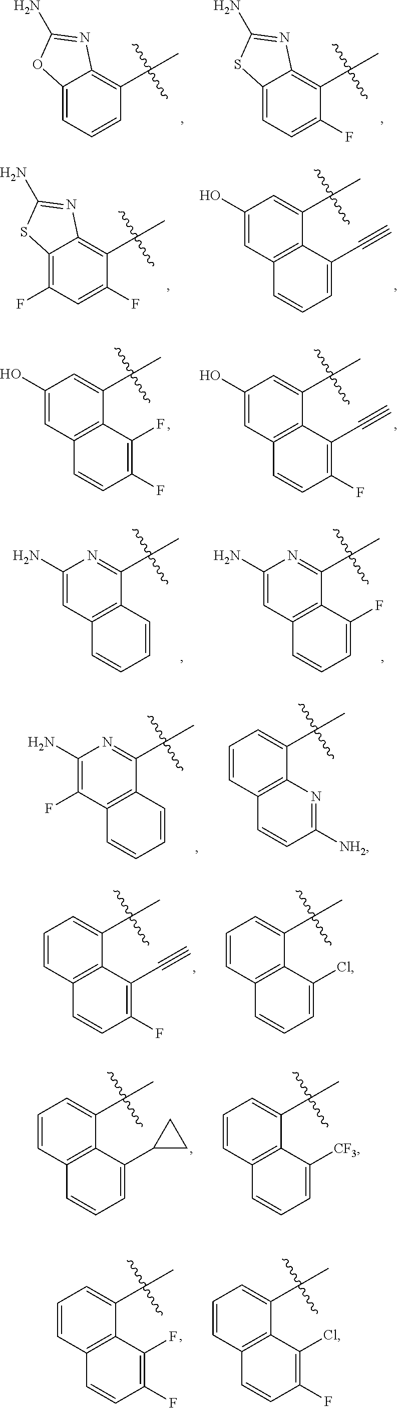

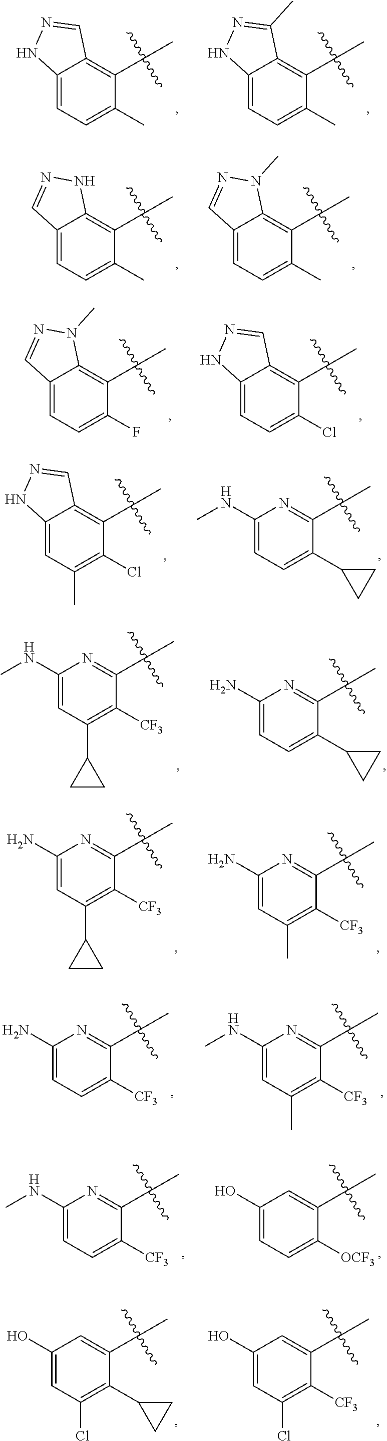

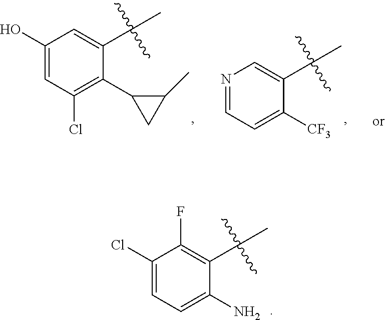

In embodiments R¹⁹ is selected from —CH═CH₂,

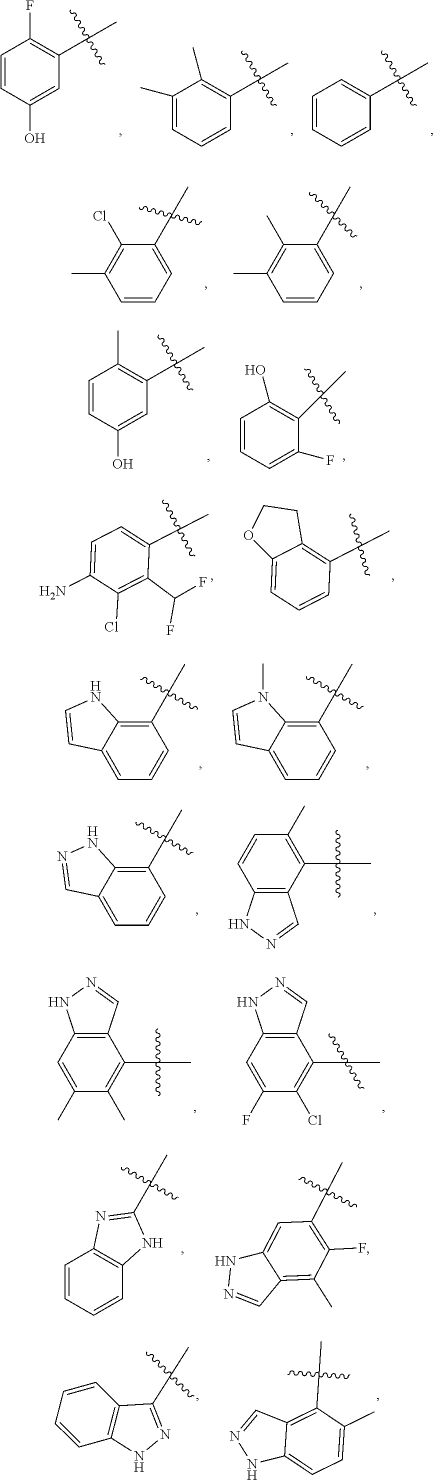

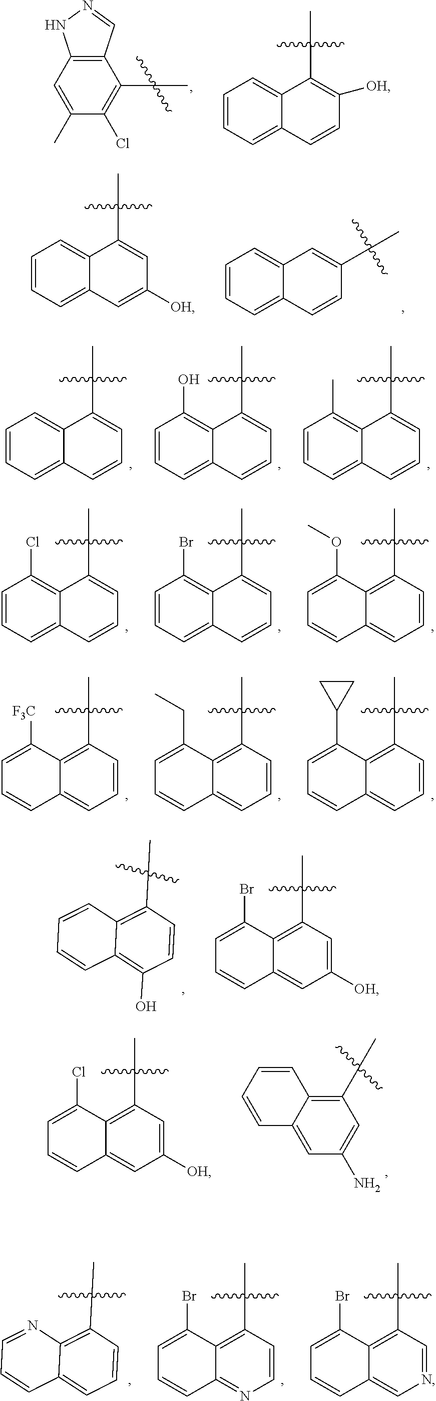

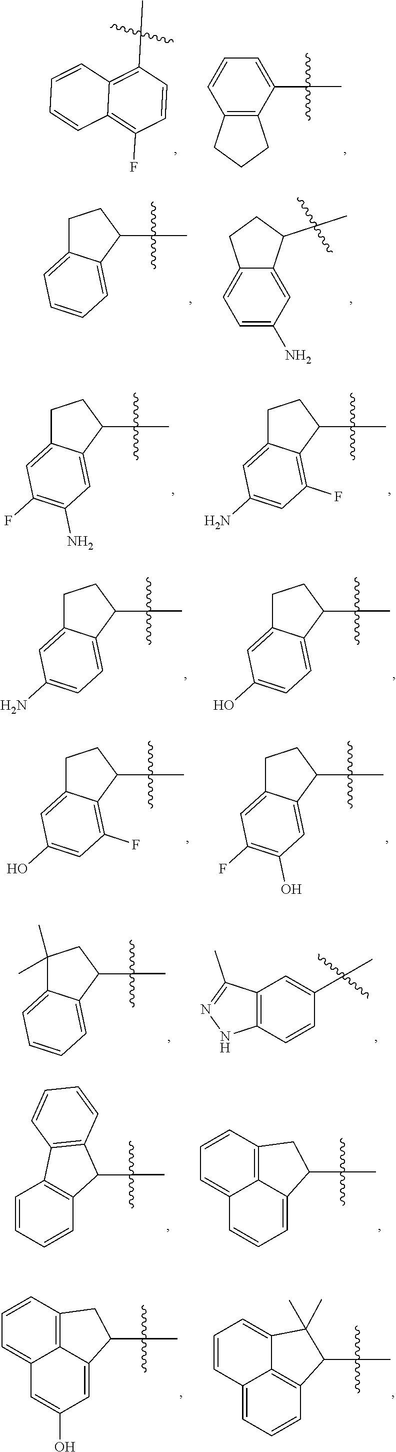

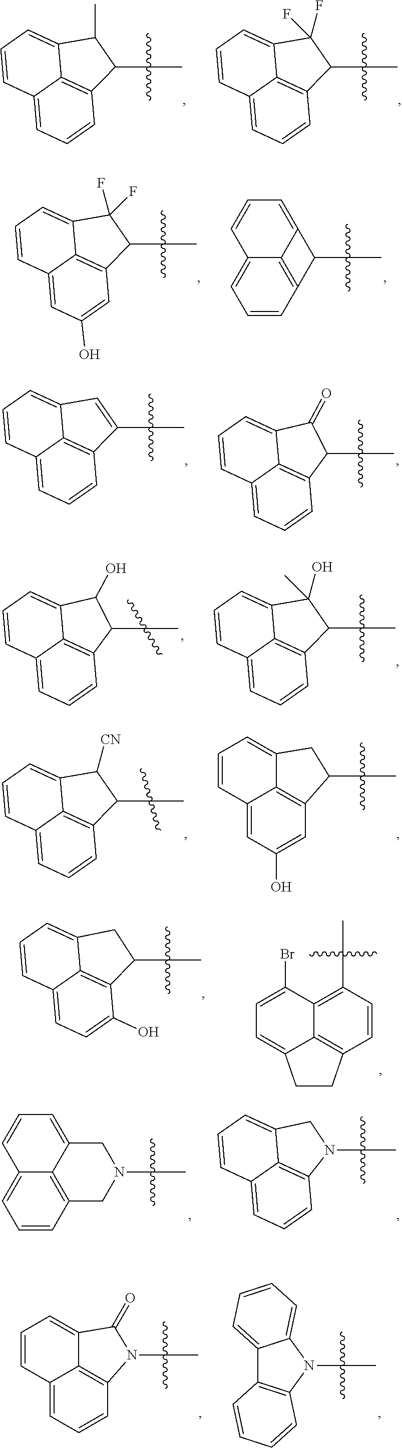

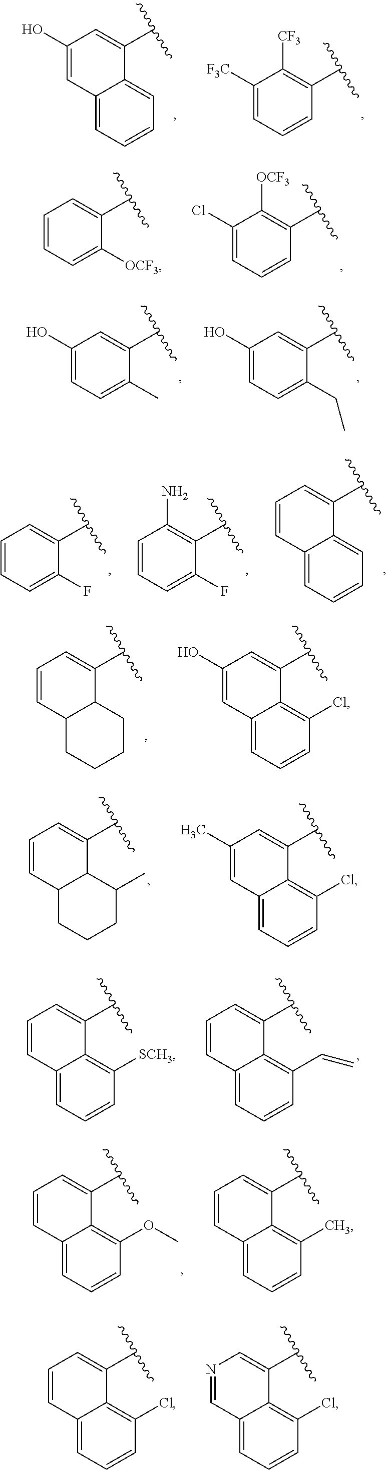

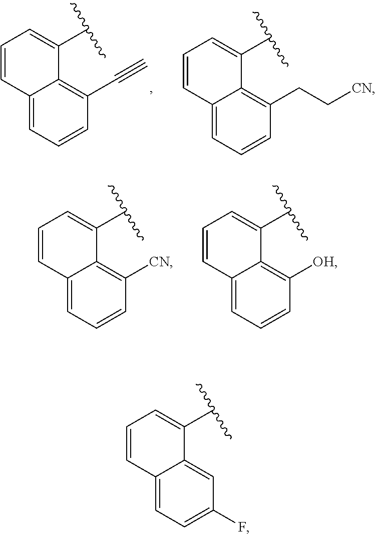

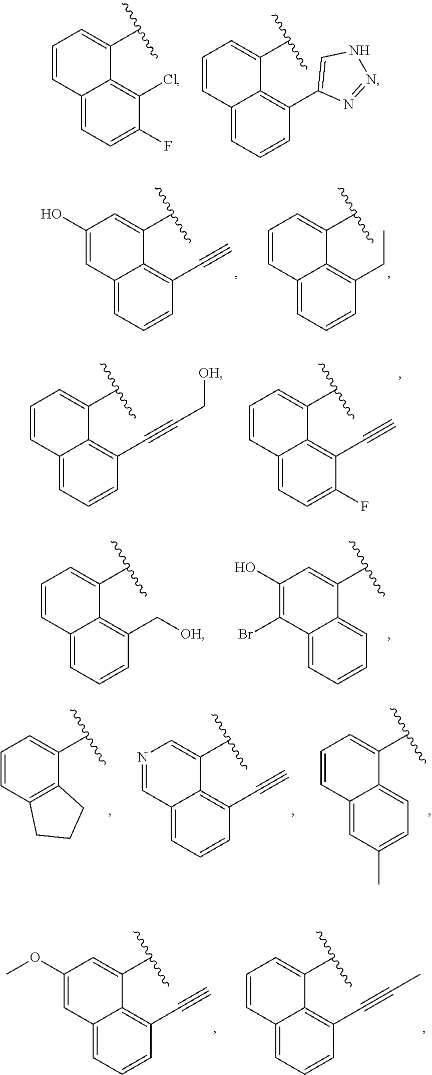

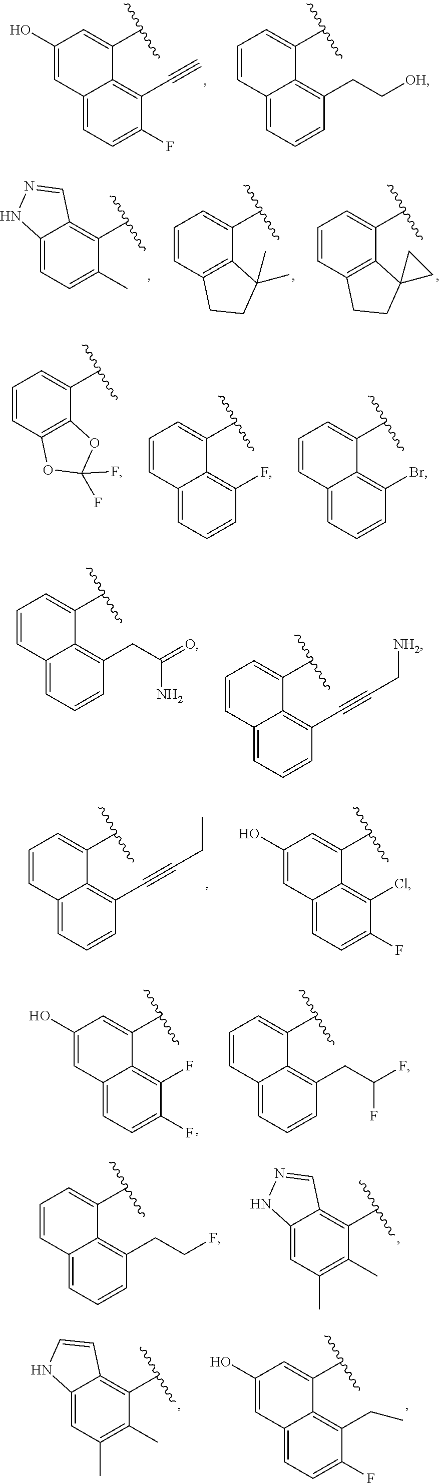

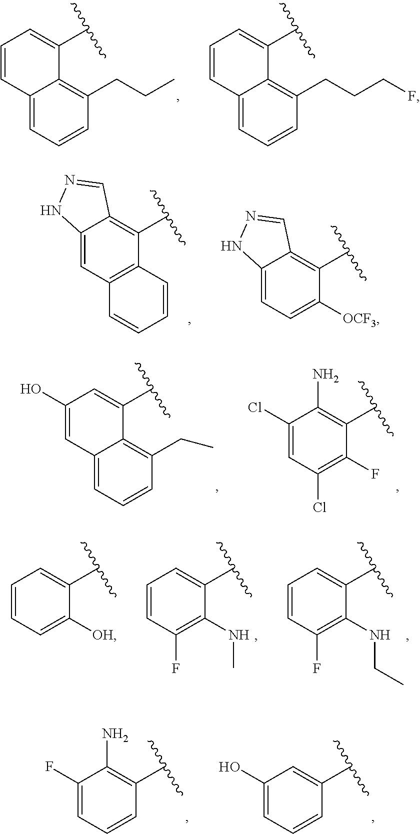

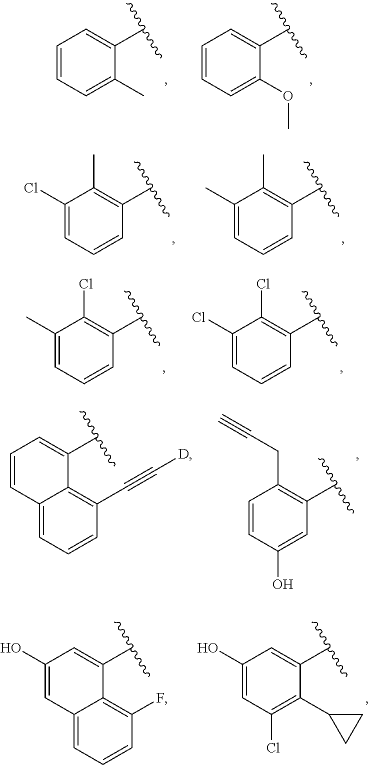

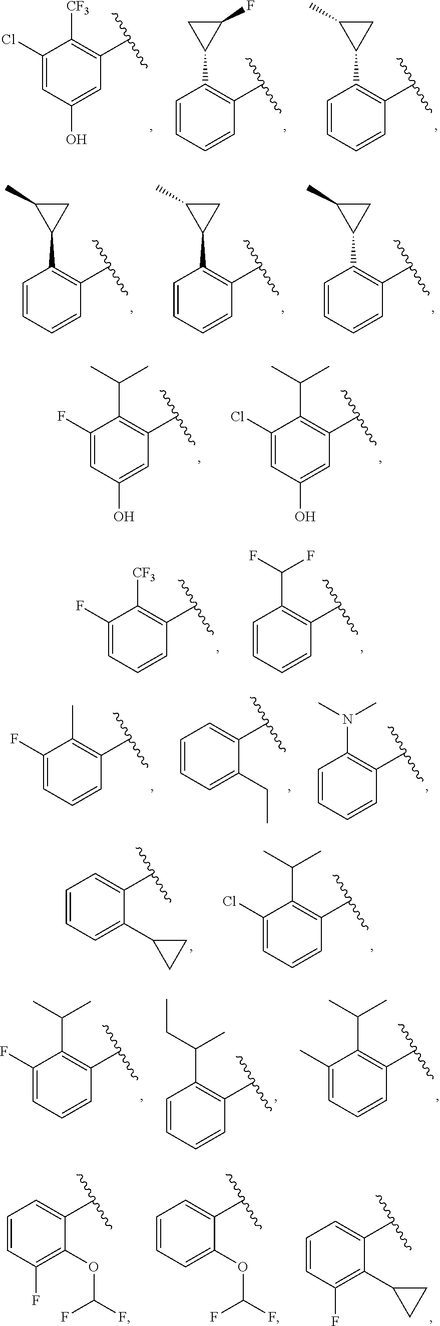

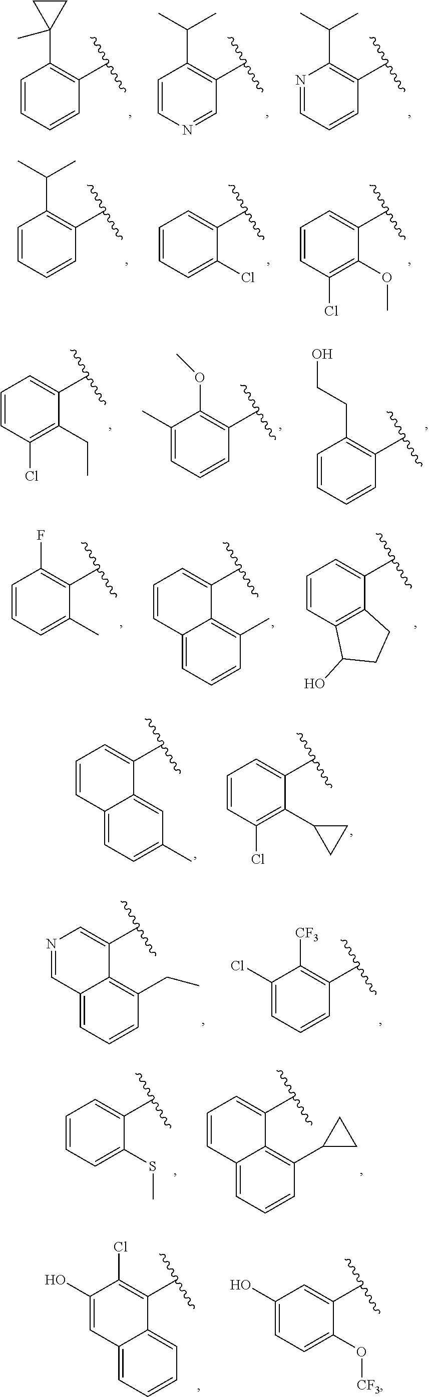

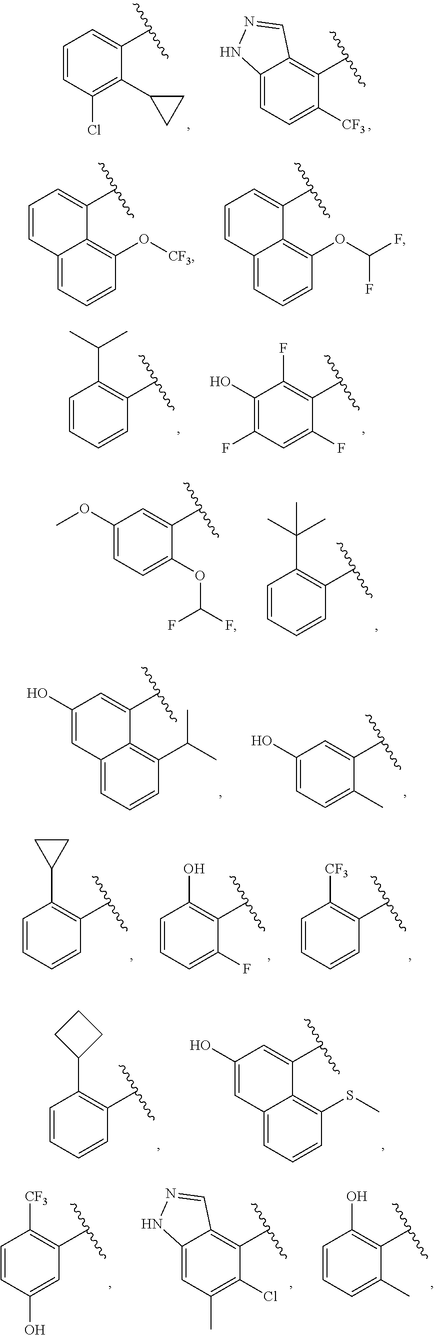

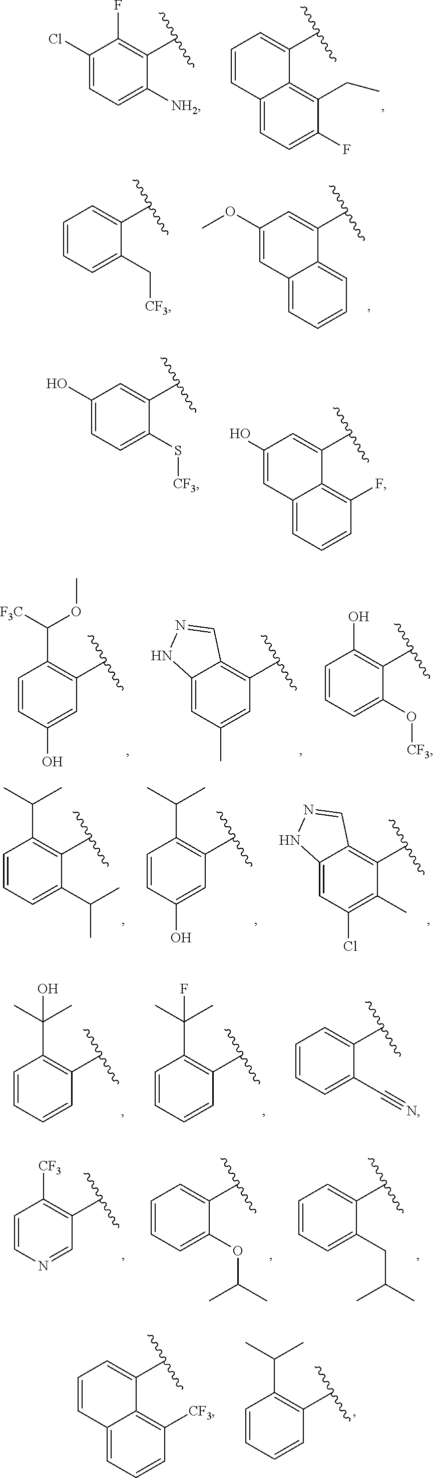

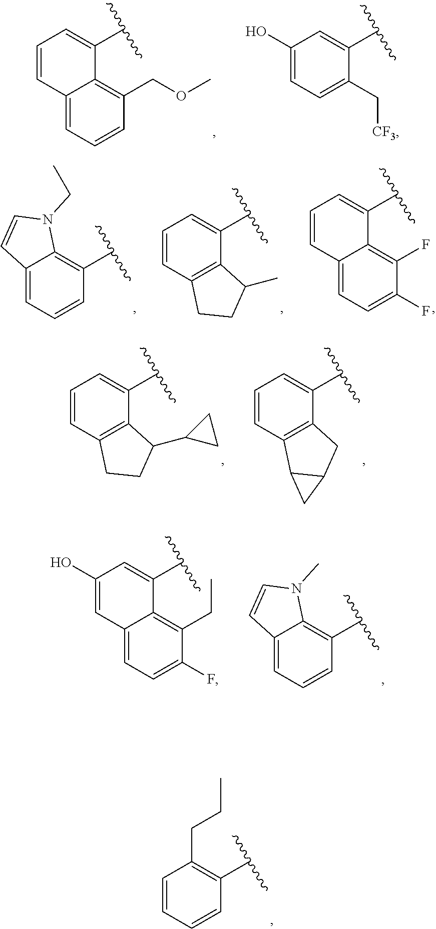

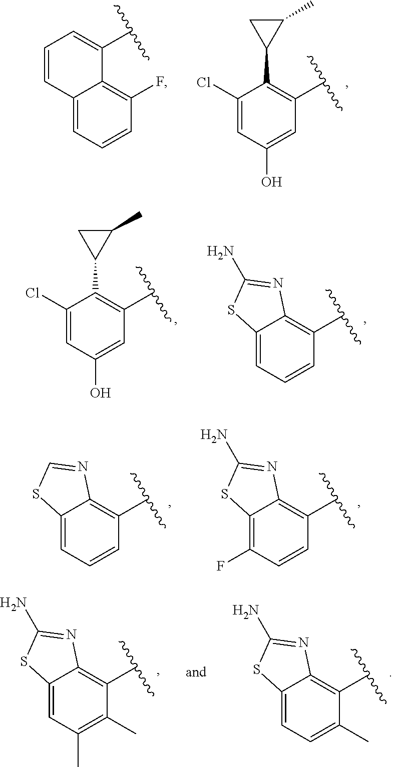

In some embodiments, R¹⁹ is selected from:

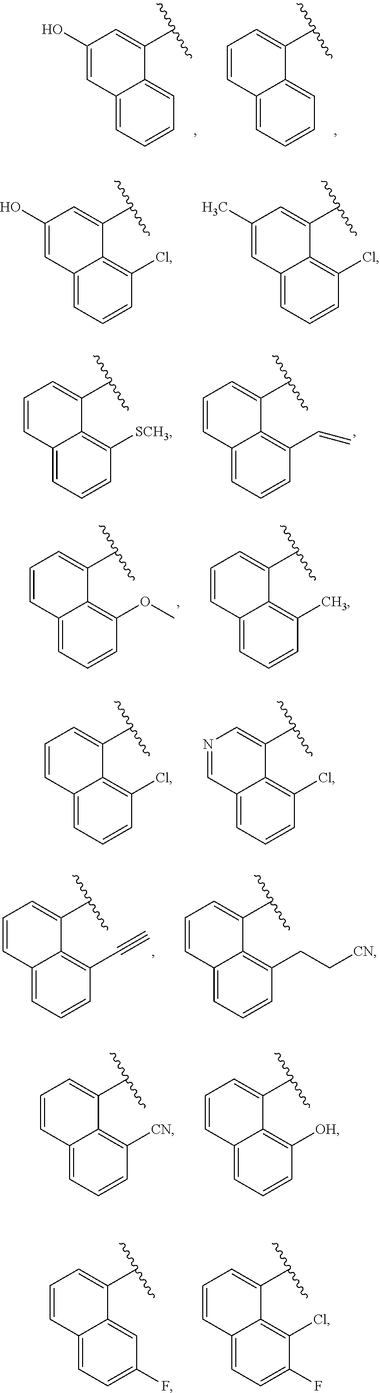

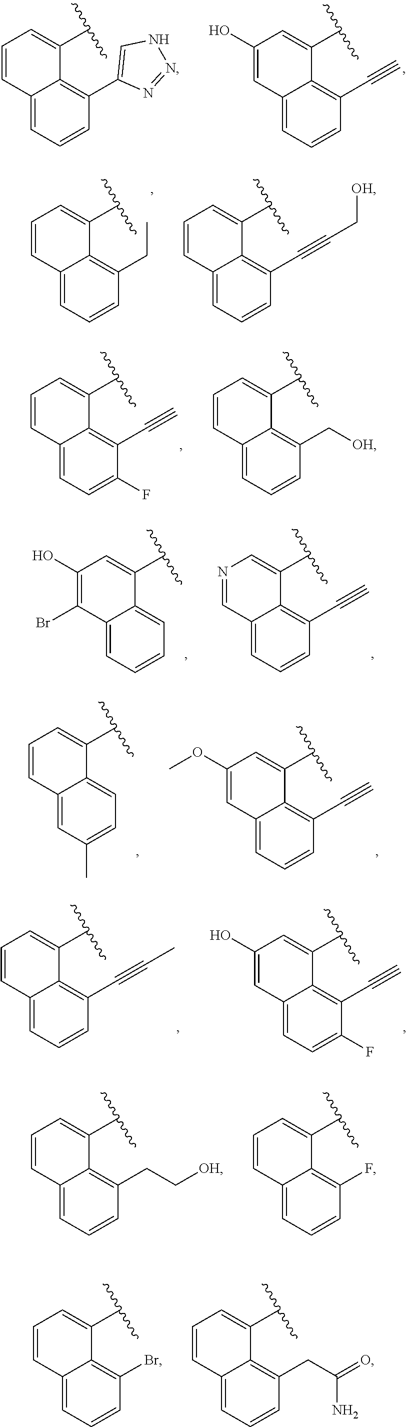

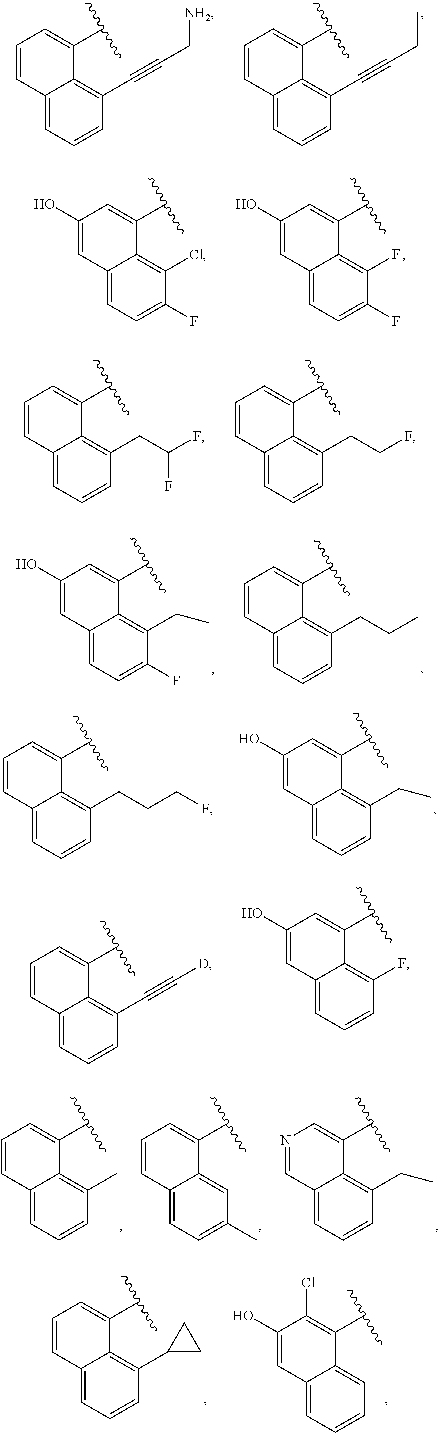

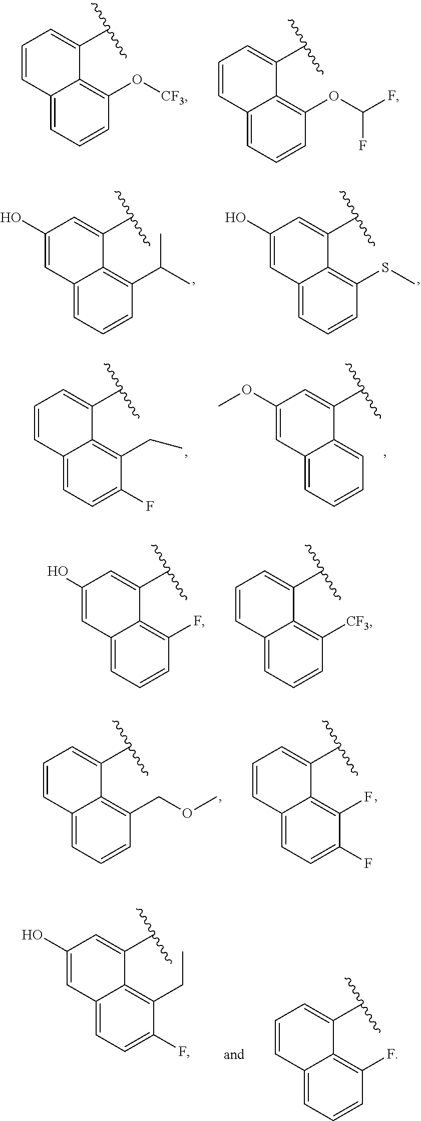

In embodiments of the compound, R¹⁹ is

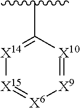

wherein X¹⁴, X¹⁵, X⁶, X, and X¹⁰ are as described herein. In embodiments of the compound, R¹⁹ is

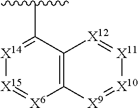

wherein X¹⁴, X¹⁵, X⁶, X⁹, X¹⁰, X¹¹, and X¹² are as described herein. In embodiments of the compound, R¹⁹ is

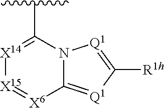

wherein X¹⁴, X¹⁵, X⁶, Q¹, and R¹) are as described herein. In embodiments of the compound, R¹⁹ is

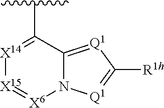

wherein X¹⁴, X¹⁵, X⁶, Q¹, and R¹⁴ are as described herein. In embodiments of the compound, R¹⁹ is

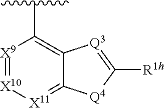

wherein X⁹, X¹⁰, X¹¹, Q³, Q⁴, and R¹⁴ are as described herein. In embodiments of the compound, R¹⁹ is

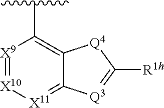

wherein X⁹, X¹⁰, X¹¹, Q³, Q⁴, and R^{1h} are as described herein. In embodiments of the compound, R¹⁹ is

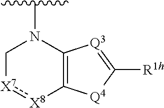

wherein X¹, X⁸, X¹², Q³, Q⁴, and R^{1h} are as described herein. In embodiments of the compound, R¹⁹ is

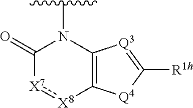

wherein X¹, X⁸, Q³, Q⁴, and R^{1h} are as described herein. In embodiments of the compound, R¹⁹ is

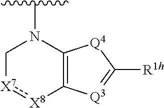

wherein X¹, X⁸, Q³, Q⁴, and R^{1h} are as described herein. In embodiments of the compound, R¹⁹ is

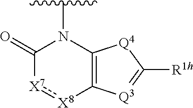

wherein X¹, X⁸, Q³, Q⁴, and R^{1h} are as described herein. In embodiments of the compound, R¹⁹ is

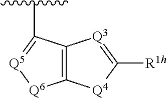

wherein Q³, Q⁴, Q⁵, Q⁶, and R^{1h} are as described herein. In embodiments of the compound, R¹⁹ is

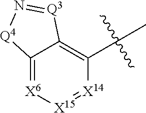

wherein X¹⁴, X¹⁵, X⁶, Q³, and Q⁴ are as described herein. In embodiments of the compound, R¹⁹ is

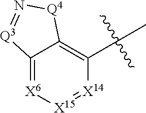

wherein X¹⁴, X¹⁵, X⁶, Q³, and Q⁴ are as described herein. In embodiments of the compound, R¹⁹ is

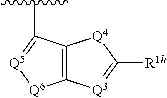

wherein, Q³, Q⁴, Q⁵, Q⁶, and R^{1h} are as described herein. In embodiments of the compound, R¹⁹ is

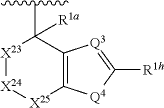

wherein Q³, Q⁴, X²³, X²⁴, X²⁵, R^{1a} are R^{1h} are as described herein.

In embodiments of the compound, R¹⁹ is

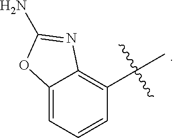 [SMILES: *c1cccc2oc(N)nc12]

In some embodiments of the compound, R¹⁹ is

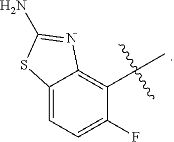 [SMILES: *c1c(F)ccc2sc(N)nc12]

In further embodiments of the compound, R¹⁹ is

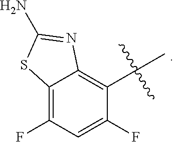 [SMILES: *c1c(F)cc(F)c2sc(N)nc12]

In additional embodiments of the compound, R¹⁹ is

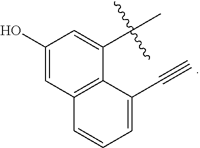 [SMILES: *c1cc(O)cc2cccc(C#C)c12]

In select embodiments of the compound, R¹⁹ is

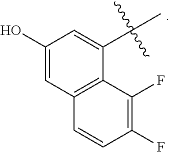 [SMILES: *c1cc(O)cc2ccc(F)c(F)c12]

In embodiments of the compound, R¹⁹ is

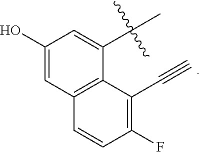 [SMILES: *c1cc(O)cc2ccc(F)c(C#C)c12]

In some embodiments of the compound, R¹⁹ is

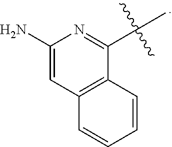 [SMILES: *c1nc(N)cc2ccccc12]

In some embodiments of the compound, R¹⁹ is

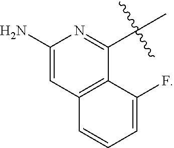 [SMILES: *c1nc(N)cc2cccc(F)c12]

In further embodiments of the compound, R¹⁹ is

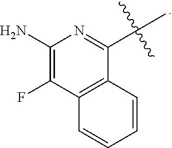 [SMILES: *c1nc(N)c(F)c2ccccc12]

In select embodiments of the compound, R¹⁹ is

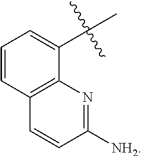 [SMILES: *c1cccc2ccc(N)nc12]

In embodiments of the compound, R¹⁹ is

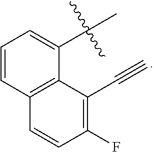 [SMILES: *c1cccc2ccc(F)c(C#C)c12]

In some embodiments of the compound, R¹⁹ is

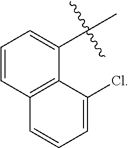 [SMILES: *c1cccc2cccc(Cl)c12]

In additional embodiments of the compound, R¹⁹ is

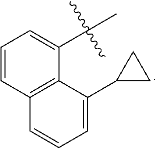 [SMILES: *c1cccc2cccc(C3CC3)c12]

In some embodiments of the compound, R¹⁹ is

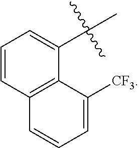 [SMILES: *c1cccc2cccc(C(F)(F)F)c12]

In further embodiments of the compound, R¹⁹ is

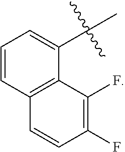 [SMILES: *c1cccc2ccc(F)c(F)c12]

In embodiments of the compound, R¹⁹ is

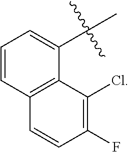 [SMILES: *c1cccc2ccc(F)c(Cl)c12]

In select embodiments of the compound, R¹⁹ is

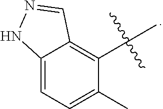 [SMILES: *c1c(C)ccc2[nH]ncc12]

In some embodiments of the compound, R¹⁹ is

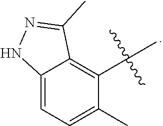 [SMILES: *c1c(C)ccc2[nH]nc(C)c12]

In additional embodiments of the compound, R¹⁹ is

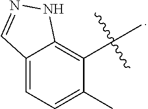 [SMILES: *c1c(C)ccc2cn[nH]c12]

In embodiments of the compound, R¹⁹ is

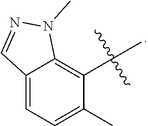 [SMILES: *c1c(C)ccc2cnn(C)c12]

In embodiments of the compound, R¹⁹ is

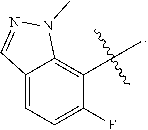 [SMILES: *c1c(F)ccc2cnn(C)c12]

In further embodiments of the compound, R¹⁹ is

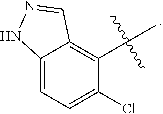 [SMILES: *c1c(Cl)ccc2[nH]ncc12]

In select embodiments of the compound, R¹⁹ is

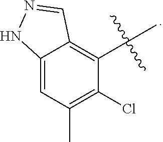 [SMILES: *c1c(Cl)c(C)cc2[nH]ncc12]

In some embodiments of the compound, R¹⁹ is

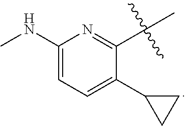 [SMILES: *c1nc(NC)ccc1C1CC1]

In additional embodiments of the compound, R¹⁹ is

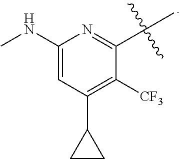 [SMILES: *c1nc(NC)cc(C2CC2)c1C(F)(F)F]

In embodiments of the compound, R¹⁹ is

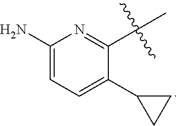 [SMILES: *c1nc(N)ccc1C1CC1]

In some embodiments of the compound, R¹⁹ is

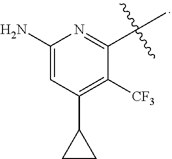 [SMILES: *c1nc(N)cc(C2CC2)c1C(F)(F)F]

In further embodiments of the compound, R¹⁹ is

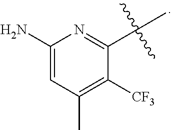 [SMILES: *c1nc(N)cc(C)c1C(F)(F)F]

In select embodiments of the compound, R¹⁹ is

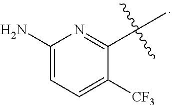 [SMILES: *c1nc(N)ccc1C(F)(F)F]

In some embodiments of the compound, R¹⁹ is

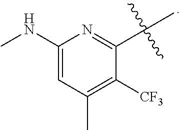 [SMILES: *c1nc(NC)cc(C)c1C(F)(F)F]

In embodiments of the compound, R¹⁹ is

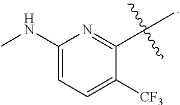 [SMILES: *c1nc(NC)ccc1C(F)(F)F]

In some embodiments of the compound, R¹⁹ is

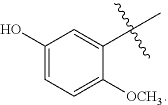 [SMILES: *c1cc(O)ccc1OC]

In additional embodiments of the compound, R¹⁹ is

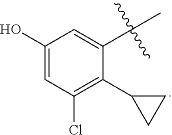 [SMILES: *c1cc(O)cc(Cl)c1C1CC1]

In further embodiments of the compound, R¹⁹ is

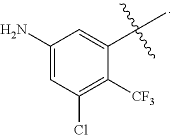 [SMILES: *c1cc(N)cc(Cl)c1C(F)(F)F]

In select embodiments of the compound, R¹⁹ is

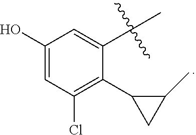 [SMILES: *c1cc(O)cc(Cl)c1C1CC1C]

In embodiments of the compound, R¹⁹ is

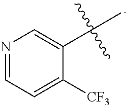 [SMILES: *c1cnccc1C(F)(F)F]

In some embodiments of the compound, R¹⁹ is

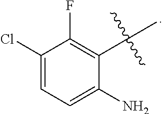 [SMILES: *c1c(N)ccc(Cl)c1F]

In some embodiments of the compound, R¹⁹ is

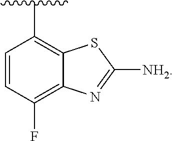 [SMILES: *c1ccc(F)c2nc(N)sc12]

In some embodiments of the compound, R¹⁹ is

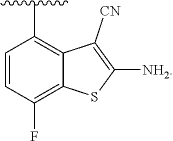 [SMILES: *c1ccc(F)c2sc(N)c(C#N)c12]

In some embodiments of the compound, R¹⁹ is

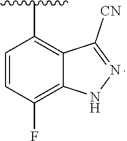 [SMILES: *c1ccc(F)c2[nH]nc(C#N)c12]

In some embodiments of the compound, R¹⁹ is

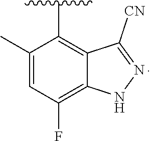 [SMILES: *c1c(C)cc(F)c2[nH]nc(C#N)c12]

In some embodiments of the compound, R¹⁹ is

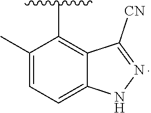 [SMILES: *c1c(C)ccc2[nH]nc(C#N)c12]

In some embodiments of the compound, R¹⁹ is

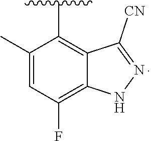 [SMILES: *c1c(C)cc(F)c2[nH]nc(C#N)c12]

In some embodiments of the compound, R¹⁹ is

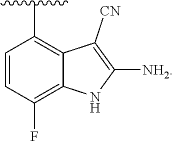 [SMILES: *c1ccc(F)c2[nH]c(N)c(C#N)c12]

In some embodiments of the compound, R¹⁹ is

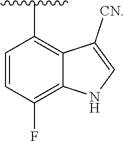 [SMILES: *c1ccc(F)c2[nH]cc(C#N)c12]

In some embodiments of the compound, R¹⁹ is

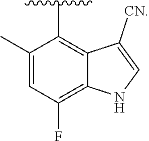 [SMILES: *c1c(C)cc(F)c2[nH]cc(C#N)c12]

In some embodiments of the compound, R¹⁹ is

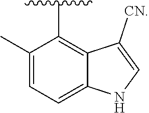 [SMILES: *c1c(C)ccc2[nH]cc(C#N)c12]

In some embodiments of the compound, R¹⁹ is

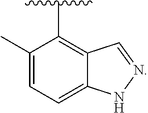 [SMILES: *c1c(C)ccc2[nH]ncc12]

In some embodiments of the compound, R¹⁹ is

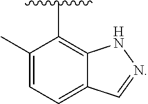 [SMILES: *c1c(C)ccc2cn[nH]c12]

In embodiments of the compound, R¹⁹ is

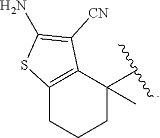 [SMILES: *C1(C)CCCc2sc(N)c(C#N)c21]

In embodiments of the compound, R¹⁹ is

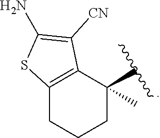 [SMILES: *[C@@]1(C)CCCc2sc(N)c(C#N)c21]

In embodiments of the compound, R¹⁹ is

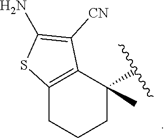 [SMILES: *[C@]1(C)CCCc2sc(N)c(C#N)c21]

In embodiments of the compound, R¹⁹ is

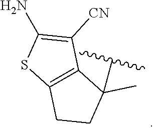 [SMILES: *C1(C)CCc2sc(N)c(C#N)c21]

In embodiments of the compound, R¹⁹ is

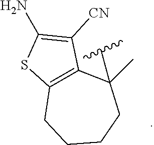 [SMILES: *C1(C)CCCCc2sc(N)c(C#N)c21]

In embodiments of the compound, R¹⁹ is

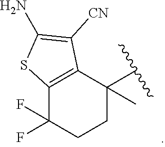 [SMILES: *C1(C)CCC(F)(F)c2sc(N)c(C#N)c21]

In embodiments of the compound, R¹⁹ is

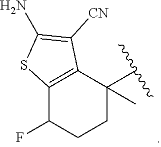 [SMILES: *C1(C)CCC(F)c2sc(N)c(C#N)c21]

In embodiments of the compound, R¹⁹ is

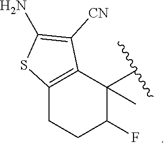 [SMILES: *C1(C)c2c(sc(N)c2C#N)CCC1F]

In embodiments of the compound, R¹⁹ is

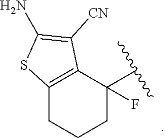 [SMILES: *C1(F)CCCc2sc(N)c(C#N)c21]

In embodiments of the compound, R¹⁹ is

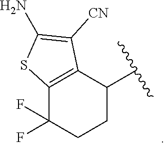 [SMILES: *C1CCC(F)(F)c2sc(N)c(C#N)c21]

In embodiments of the compound, R¹⁹ is

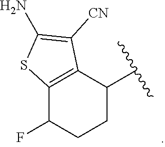 [SMILES: *C1CCC(F)c2sc(N)c(C#N)c21]

In embodiments of the compound, R¹⁹ is

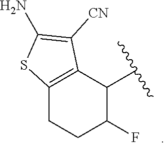 [SMILES: *C1c2c(sc(N)c2C#N)CCC1F]

In embodiments of the compound, R¹⁹ is

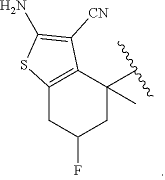 [SMILES: *C1(C)CC(F)Cc2sc(N)c(C#N)c21]

In embodiments of the compound, R¹⁹ is

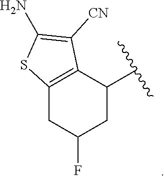 [SMILES: *C1CC(F)Cc2sc(N)c(C#N)c21]

In some embodiments, R² is selected from

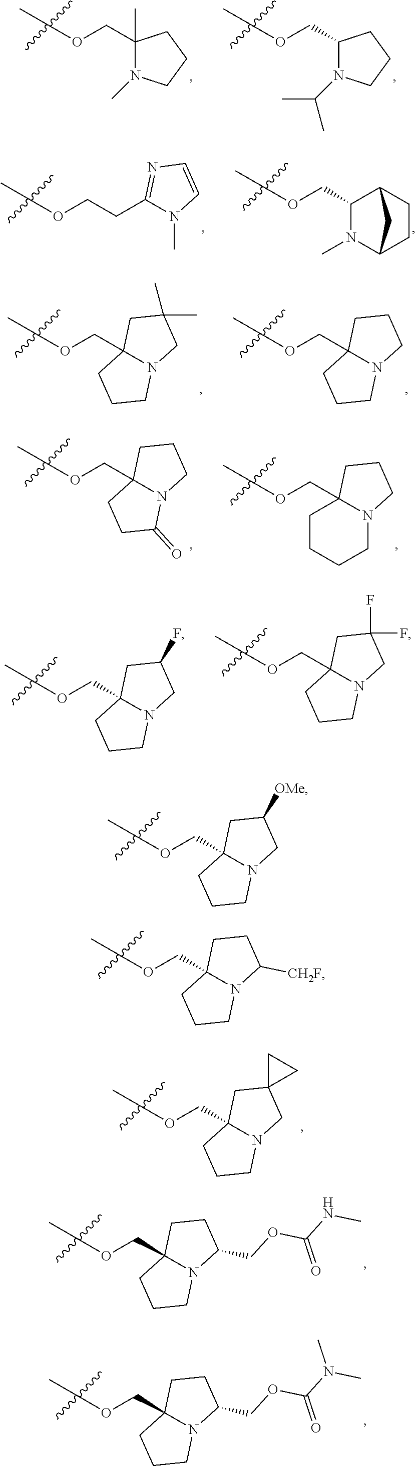

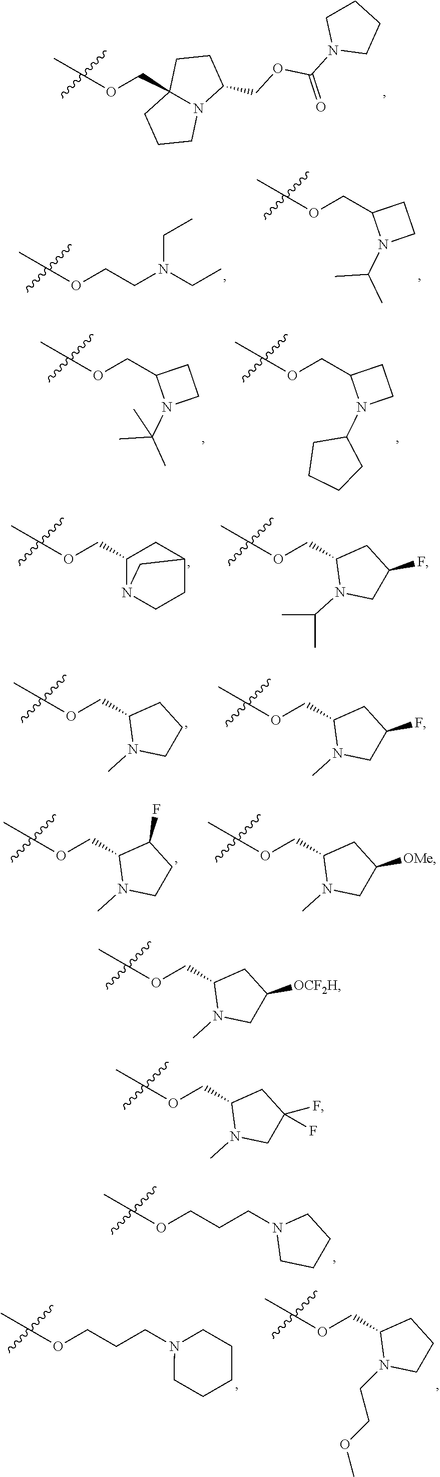

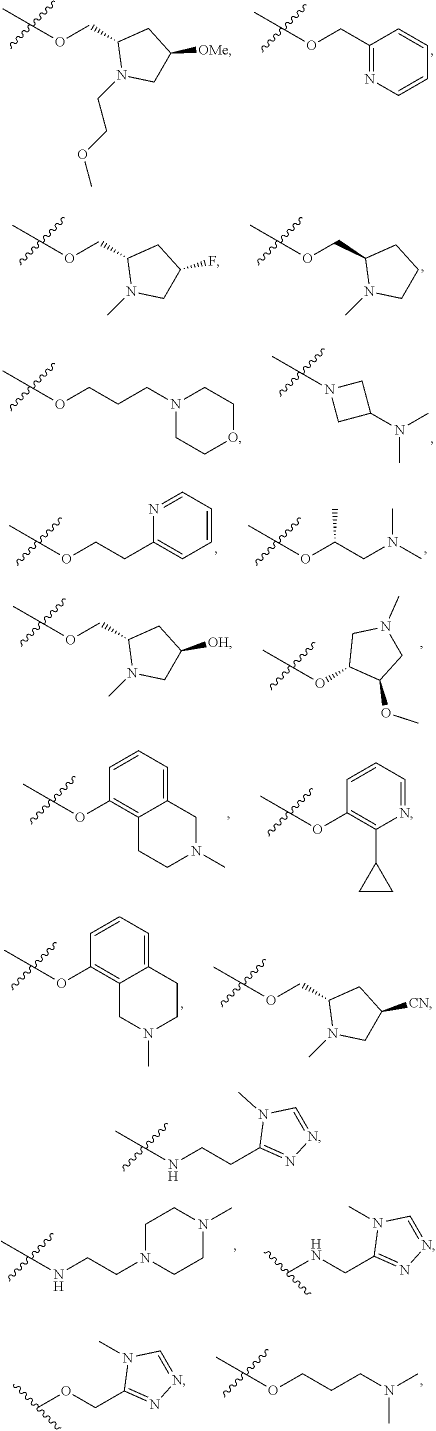

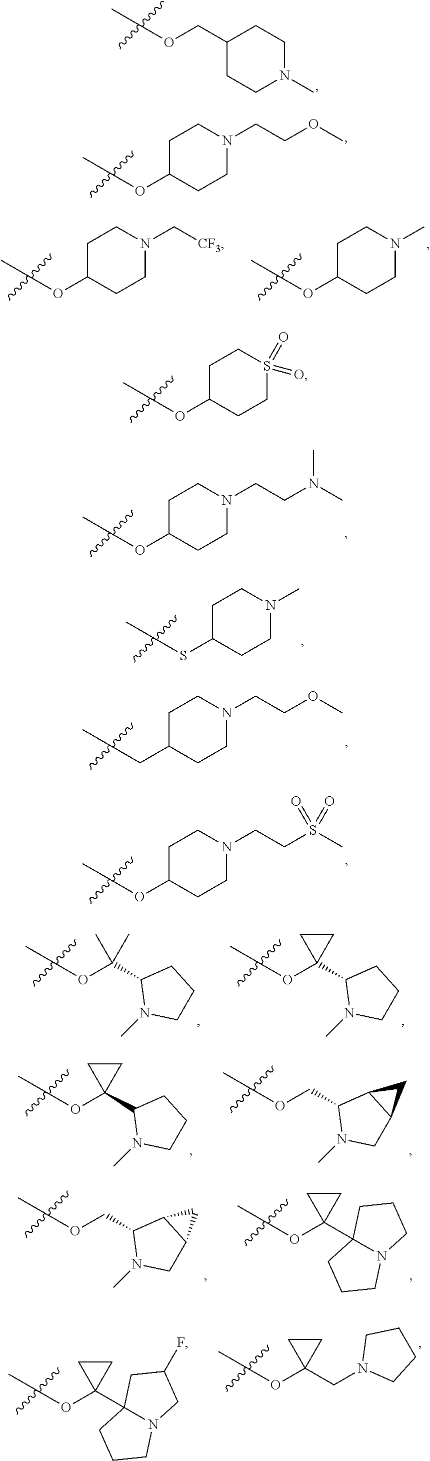

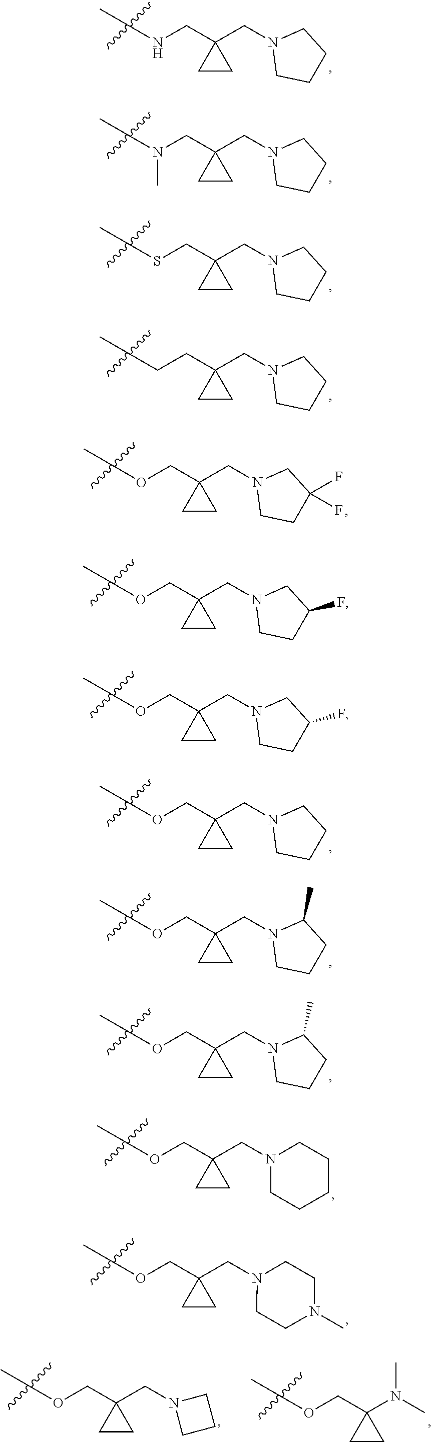

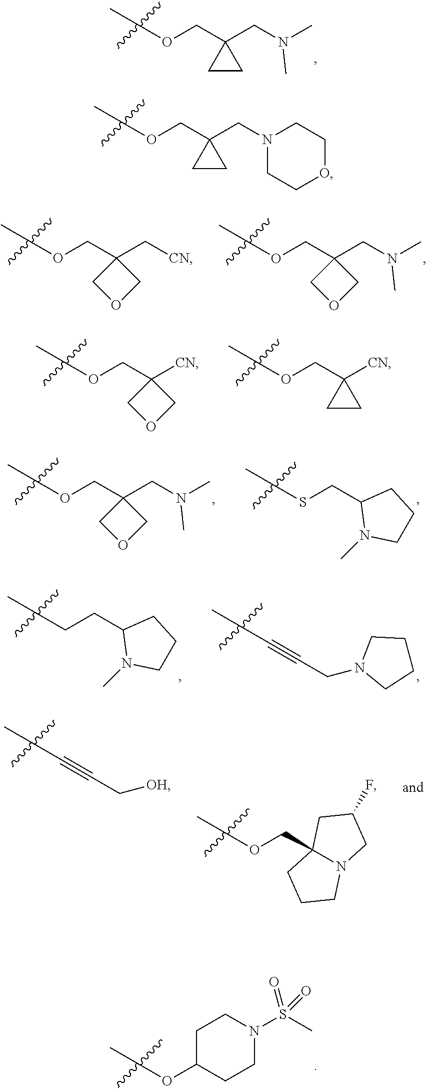

In embodiments of the compound, or a pharmaceutically acceptable salt or solvate thereof, R² is selected from

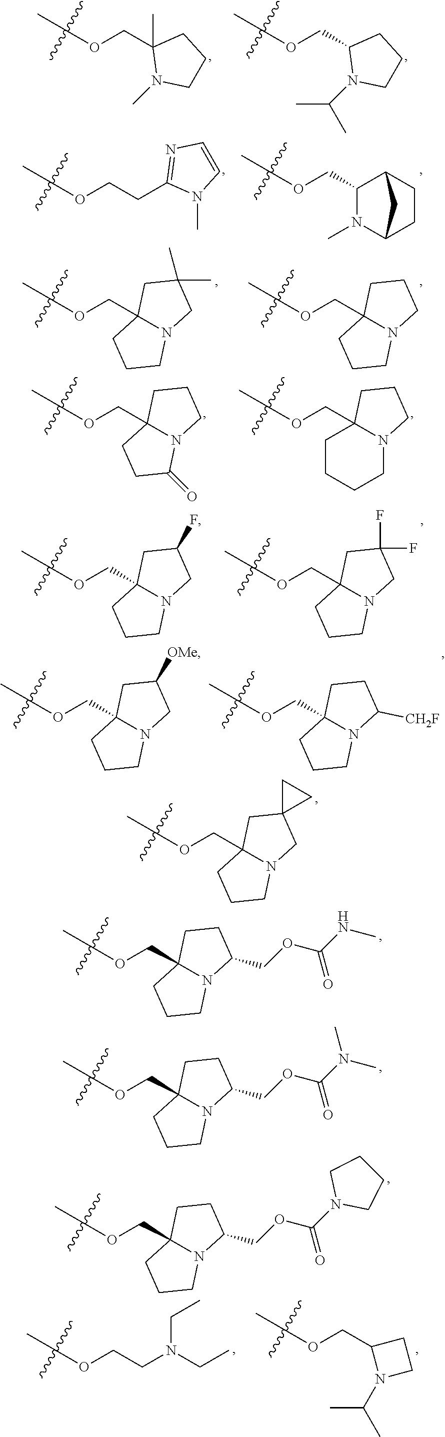

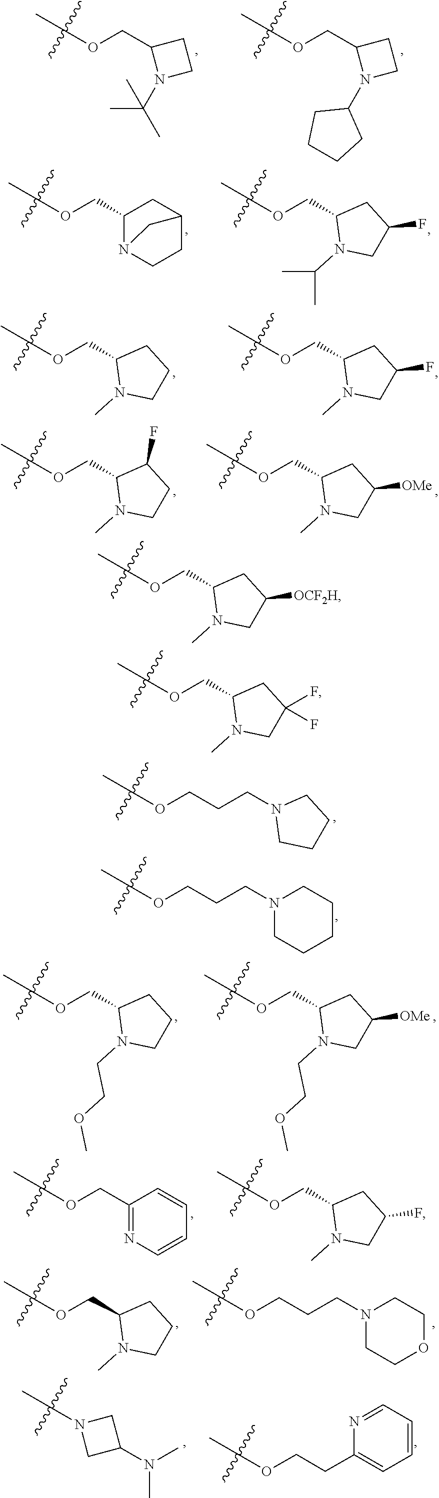

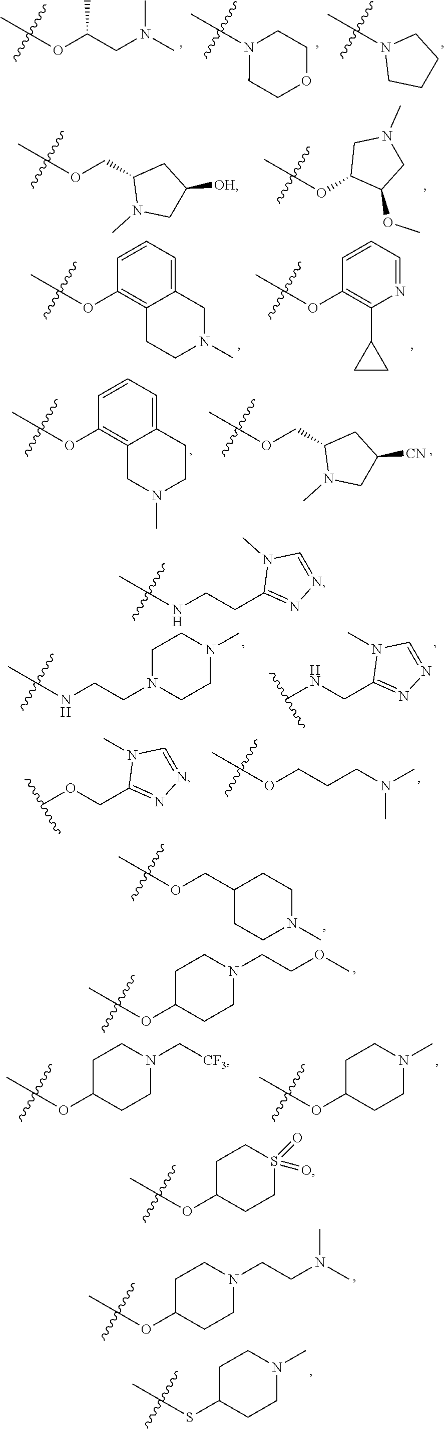

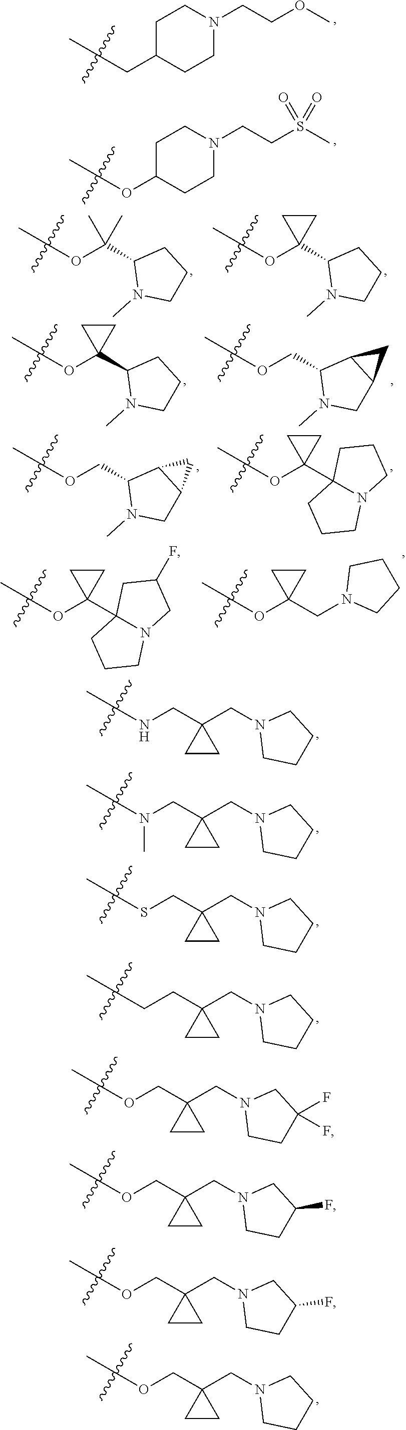

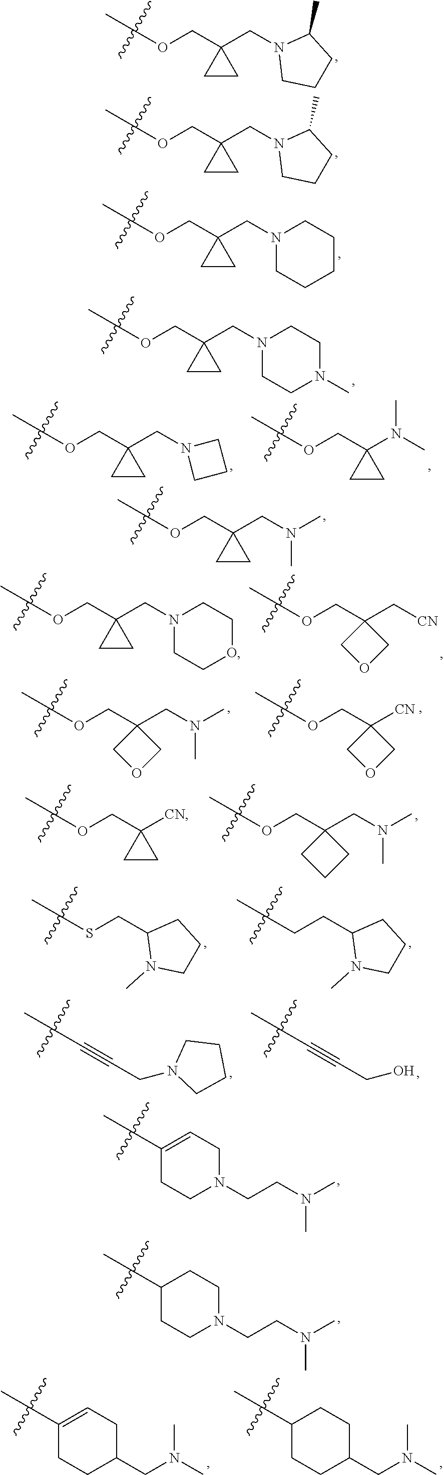

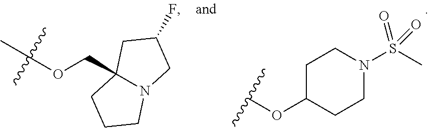 [SMILES: *OC[C@]12CCCN1C[C@@H](F)C2 ; *OC1CCN(S(C)(=O)=O)CC1]

In embodiments, R² is —O—CH₂—C₂₋₉heterocycloalkyl optionally substituted with one, two, or three R^{20b} wherein R²⁰ⁱ is independently halogen (e.g., F). In embodiments, R² is —O—CH₂—C₂₋₉heterocycloalkyl optionally substituted with one, two, or three R^{2d} wherein R^{20b} is independently C₁alkyl (e.g., methyl). In embodiments, R² is

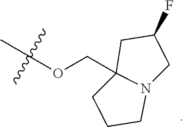 [SMILES: *OCC12CCCN1C[C@H](F)C2]

In embodiments, R² is

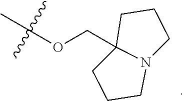 [SMILES: *OCC12CCCN1CCC2]

In embodiments, R² is

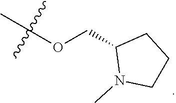 [SMILES: *OC[C@@H]1CCCN1C]

In embodiments, R² is

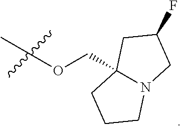 [SMILES: *OC[C@@]12CCCN1C[C@H](F)C2]

In embodiments, R² is

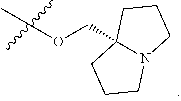

In embodiments, R² is

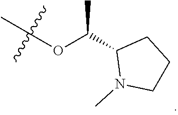 [SMILES: *O[C@@H](C)[C@@H]1CCCN1C]

In additional embodiments of the compound, R² is

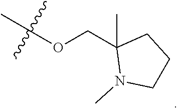 [SMILES: *OCC1(C)CCCN1C]

In embodiments of the compound, R² is

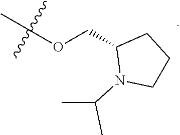 [SMILES: *OC[C@@H]1CCCN1C(C)C]

In select embodiments of the compound, R² is

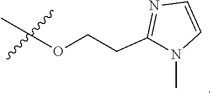 [SMILES: *OCCc1nccn1C]

In further embodiments of the compound, R² is

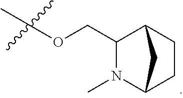 [SMILES: *OCC1[C@@H]2CC[C@@H](C2)N1C]

In some embodiments of the compound, R² is

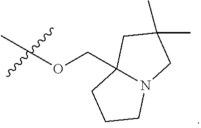 [SMILES: *OCC12CCCN1CC(C)(C)C2]

In some embodiments of the compound, R² is

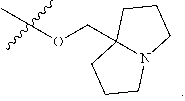 [SMILES: *OCC12CCCN1CCC2]

In embodiments of the compound, R² is

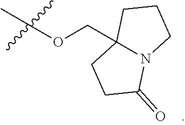 [SMILES: *OCC12CCCN1C(=O)CC2]

In additional embodiments of the compound, R² is

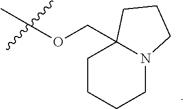 [SMILES: *OCC12CCCCN1CCC2]

In select embodiments of the compound, R² is

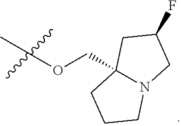 [SMILES: *OC[C@@]12CCCN1C[C@H](F)C2]

In further embodiments of the compound, R² is

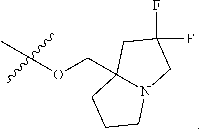 [SMILES: *OCC12CCCN1CC(F)(F)C2]

In some embodiments of the compound, R² is

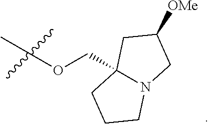 [SMILES: *OC[C@@]12CCCN1C[C@H](OC)C2]

In embodiments of the compound, R² is

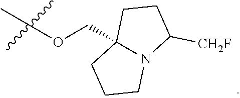

In some embodiments of the compound, R² is

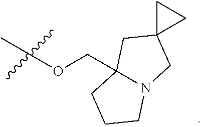 [SMILES: *OCC12CCCN1CC1(CC1)C2]

In additional embodiments of the compound, R² is

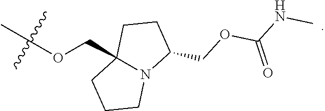 [SMILES: *OC[C@]12CCCN1[C@@H](COC(=O)NC)CC2]

In select embodiments of the compound, R² is

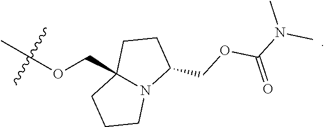 [SMILES: *OC[C@]12CCCN1[C@@H](COC(=O)N(C)C)CC2]

In further embodiments of the compound, R² is

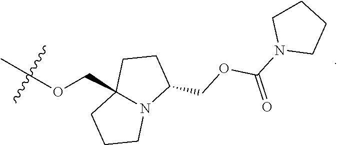 [SMILES: *OC[C@]12CCCN1[C@@H](COC(=O)N1CCCC1)CC2]

In embodiments of the compound, R² is

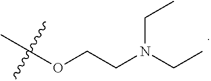 [SMILES: *OCCN(CC)CC]

In some embodiments of the compound, R² is

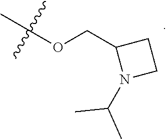 [SMILES: *OCC1CCN1C(C)C]

In embodiments of the compound, R² is

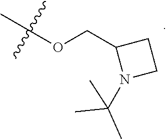 [SMILES: *OCC1CCN1C(C)(C)C]

In additional embodiments of the compound, R² is

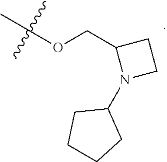 [SMILES: *OCC1CCN1C1CCCC1]

In select embodiments of the compound, R² is

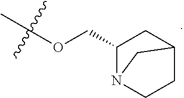 [SMILES: *OC[C@@H]1CC2CCN1C2]

In embodiments of the compound, R² is

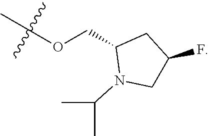 [SMILES: *OC[C@@H]1C[C@@H](F)CN1C(C)C]

In further embodiments of the compound, R² is

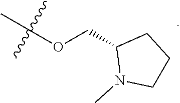 [SMILES: *OC[C@@H]1CCCN1C]

In some embodiments of the compound, R² is

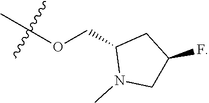 [SMILES: *OC[C@@H]1C[C@@H](F)CN1C]

In embodiments of the compound, R² is

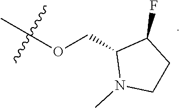 [SMILES: *OC[C@@H]1[C@@H](F)CCN1C]

In additional embodiments of the compound, R² is

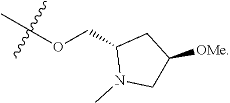 [SMILES: *OC[C@@H]1C[C@@H](OC)CN1C]

In embodiments of the compound, R² is

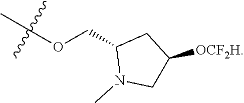

In select embodiments of the compound, R² is

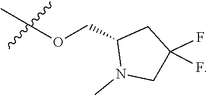 [SMILES: *OC[C@@H]1CC(F)(F)CN1C]

In further embodiments of the compound, R² is

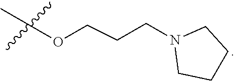 [SMILES: *OCCCN1CCCC1]

In some embodiments of the compound, R² is

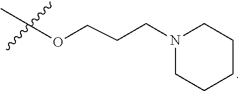 [SMILES: *OCCCN1CCCCC1]

In some embodiments of the compound, R² is

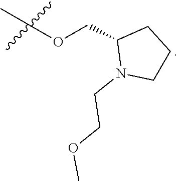 [SMILES: *OC[C@@H]1CCCN1CCOC]

In embodiments of the compound, R² is

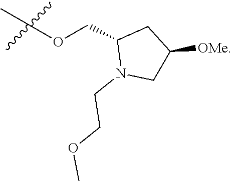 [SMILES: *OC[C@@H]1C[C@@H](OC)CN1CCOC]

In additional embodiments of the compound, R² is

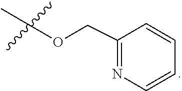 [SMILES: *OCc1ccccn1]

In select embodiments of the compound, R² is

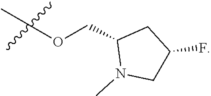 [SMILES: *OC[C@@H]1C[C@H](F)CN1C]

In further embodiments of the compound, R² is

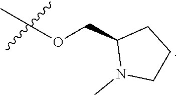 [SMILES: *OC[C@H]1CCCN1C]

In some embodiments of the compound, R² is

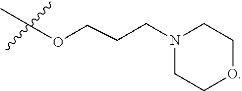 [SMILES: *OCCCN1CCOCC1]

In embodiments of the compound, R² is

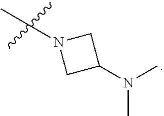 [SMILES: *N1CC(N(C)C)C1]

In embodiments of the compound, R² is

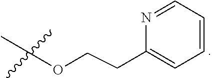 [SMILES: *OCCc1ccccn1]

In additional embodiments of the compound, R² is

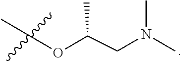 [SMILES: *O[C@H](C)CN(C)C]

In select embodiments of the compound, R² is

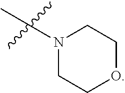 [SMILES: *N1CCOCC1]

In further embodiments of the compound, R² is

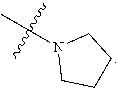 [SMILES: *N1CCCC1]

In embodiments of the compound, R² is

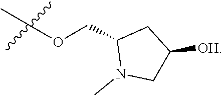 [SMILES: *OC[C@@H]1C[C@@H](O)CN1C]

In some embodiments of the compound, R² is

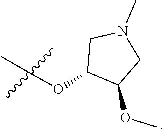 [SMILES: *O[C@@H]1CN(C)C[C@H]1OC]

In some embodiments of the compound, R² is

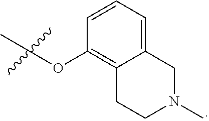 [SMILES: *Oc1cccc2c1CCN(C)C2]

In additional embodiments of the compound, R² is

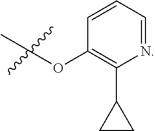 [SMILES: *Oc1cccnc1C1CC1]

In select embodiments of the compound, R² is

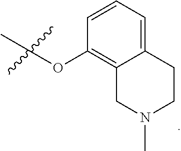 [SMILES: *Oc1cccc2c1CN(C)CC2]

In embodiments of the compound, R² is

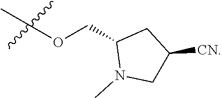 [SMILES: *OC[C@@H]1C[C@@H](C#N)CN1C]

In further embodiments of the compound, R² is

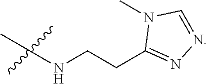 [SMILES: *NCCc1nncn1C]

In some embodiments of the compound, R² is

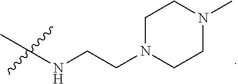 [SMILES: *NCCN1CCN(C)CC1]

In embodiments of the compound, R² is

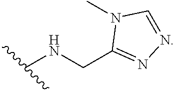 [SMILES: *NCc1nncn1C]

In additional embodiments of the compound, R² is

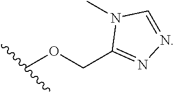 [SMILES: *OCc1nncn1C]

In embodiments of the compound, R² is

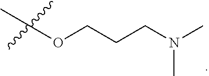 [SMILES: *OCCCN(C)C]

In select embodiments of the compound, R² is

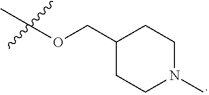 [SMILES: *OCC1CCN(C)CC1]

In further embodiments of the compound, R² is

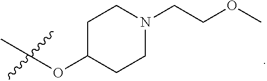 [SMILES: *OC1CCN(CCOC)CC1]

In some embodiments of the compound, R² is

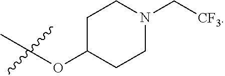 [SMILES: *OC1CCN(CC(F)(F)F)CC1]

In embodiments of the compound, R² is

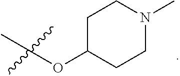 [SMILES: *OC1CCN(C)CC1]

In embodiments of the compound, R² is

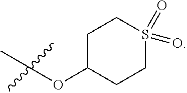 [SMILES: *OC1CCS(=O)(=O)CC1]

In additional embodiments of the compound, R² is

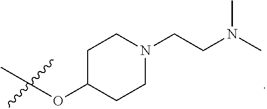 [SMILES: *OC1CCN(CCN(C)C)CC1]

In select embodiments of the compound, R² is

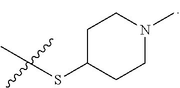 [SMILES: *SC1CCN(C)CC1]

In further embodiments of the compound, R² is

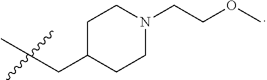 [SMILES: *CC1CCN(CCOC)CC1]

In some embodiments of the compound, R² is

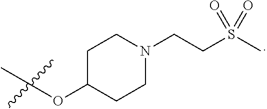 [SMILES: *OC1CCN(CCS(C)(=O)=O)CC1]

In some embodiments of the compound, R² is

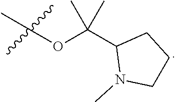 [SMILES: *OC(C)(C)C1CCCN1C]

In embodiments of the compound, R² is

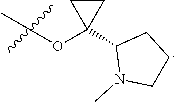 [SMILES: *OC1([C@@H]2CCCN2C)CC1]

In additional embodiments of the compound, R² is

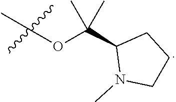 [SMILES: *OC(C)(C)[C@H]1CCCN1C]

In select embodiments of the compound, R² is

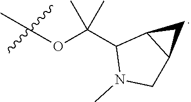 [SMILES: *OC(C)(C)C1[C@H]2C[C@H]2CN1C]

In further embodiments of the compound, R² is

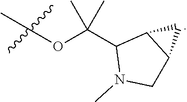 [SMILES: *OC(C)(C)C1[C@@H]2C[C@@H]2CN1C]

In some embodiments of the compound, R² is

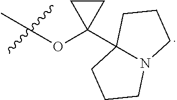 [SMILES: *OC1(C23CCCN2CCC3)CC1]

In embodiments of the compound, R² is

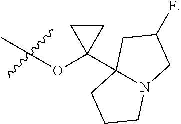 [SMILES: *OC1(C23CCCN2CC(F)C3)CC1]

In some embodiments of the compound, R² is

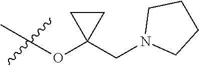 [SMILES: *OC1(CN2CCCC2)CC1]

In additional embodiments of the compound, R² is

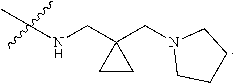 [SMILES: *NCC1(CN2CCCC2)CC1]

In select embodiments of the compound, R² is

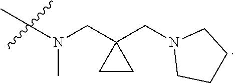 [SMILES: *N(C)CC1(CN2CCCC2)CC1]

In further embodiments of the compound, R² is

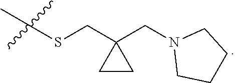 [SMILES: *SCC1(CN2CCCC2)CC1]

In embodiments of the compound, R² is

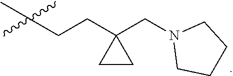 [SMILES: *CCC1(CN2CCCC2)CC1]

In some embodiments of the compound, R² is

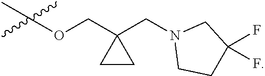 [SMILES: *OCC1(CN2CCC(F)(F)C2)CC1]

In some embodiments of the compound, R² is

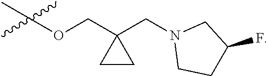 [SMILES: *OCC1(CN2CC[C@H](F)C2)CC1]

In select embodiments of the compound, R² is

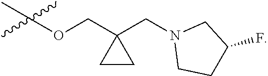 [SMILES: *OCC1(CN2CC[C@@H](F)C2)CC1]

In additional embodiments of the compound, R² is

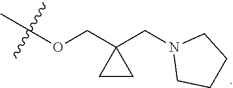 [SMILES: *OCC1(CN2CCCC2)CC1]

In embodiments of the compound, R² is

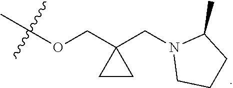 [SMILES: *OCC1(CN2CCC[C@@H]2C)CC1]

In further embodiments of the compound, R² is

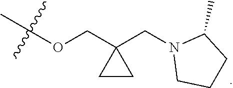 [SMILES: *OCC1(CN2CCC[C@H]2C)CC1]

In some embodiments of the compound, R² is

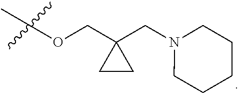 [SMILES: *OCC1(CN2CCCCC2)CC1]

In embodiments of the compound, R² is

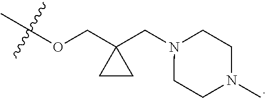 [SMILES: *OCC1(CN2CCN(C)CC2)CC1]

In select embodiments of the compound, R² is

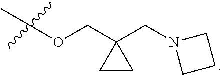 [SMILES: *OCC1(CN2CCC2)CC1]

In embodiments of the compound, R² is

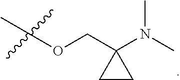 [SMILES: *OCC1(N(C)C)CC1]

In some embodiments of the compound, R² is

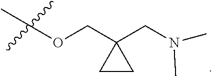 [SMILES: *OCC1(CN(C)C)CC1]

In further embodiments of the compound, R² is

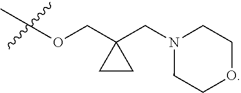 [SMILES: *OCC1(CN2CCOCC2)CC1]

In additional embodiments of the compound, R² is

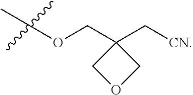 [SMILES: *OCC1(CC#N)COC1]

In some embodiments of the compound, R² is

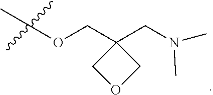 [SMILES: *OCC1(CN(C)C)COC1]

In embodiments of the compound, R² is

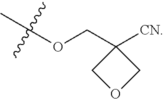 [SMILES: *OCC1(C#N)COC1]

In select embodiments of the compound, R² is

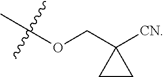 [SMILES: *OCC1(C#N)CC1]

In some embodiments of the compound, R² is

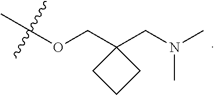 [SMILES: *OCC1(CN(C)C)CCC1]

In further embodiments of the compound, R² is

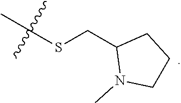 [SMILES: *SCC1CCCN1C]

In some embodiments of the compound, R² is

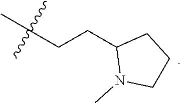 [SMILES: *CCC1CCCN1C]

In embodiments of compound, R² is

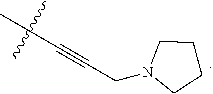 [SMILES: *C#CCN1CCCC1]

In additional embodiments of the compound, R² is

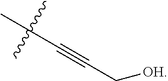 [SMILES: *C#CCO]

In select embodiments of the compound, R² is

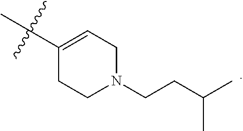 [SMILES: *C1=CCN(CCC(C)C)CC1]

In some embodiments of the compound, R² is

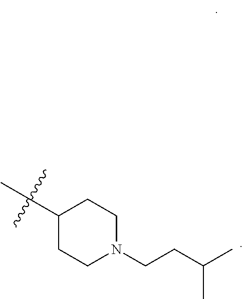 [SMILES: *C1CCN(CCC(C)C)CC1]

In further embodiments of the compound, R² is

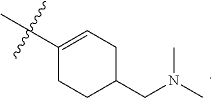 [SMILES: *C1=CCC(CN(C)C)CC1]

In embodiments of the compound, R² is

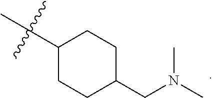 [SMILES: *C1CCC(CN(C)C)CC1]

In embodiments of the compound, R² is

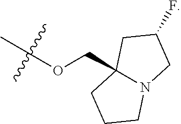 [SMILES: *OC[C@]12CCCN1C[C@@H](F)C2]

In select embodiments of the compound, R² is

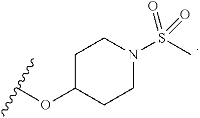 [SMILES: *OC1CCN(S(C)(=O)=O)CC1]

In embodiments, R² is

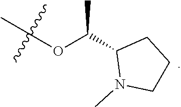 [SMILES: *O[C@@H](C)[C@@H]1CCCN1C]

In an aspect is provided a compound having the formula A-LAB-B wherein
A is a monovalent form of a compound described herein;
L^{AB} is a covalent linker bonded to A and B; and
B is a monovalent form of a degradation enhancer.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

In embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAPI (cellular inhibitor of apoptosis protein 1), APC/C (anaphase-promoting complex/cyclosome), CRBN (cereblon), CUL4-RBX1-DDB1-CRBN (CRLA^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.

In embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.

In embodiments, the degradation enhancer is a compound described in Ishida and Ciulli, SLAS Discovery 2021, Vol. 25(4) 484-502, which is incorporated by reference in its entirety for any purpose, for example VH032, VH101, VH298, thalidomide, bestatin, methyl bestatin, nutlin, idasanutlin, bardoxolone, bardoxolone methyl, indisulam (E7070), E7820, chloroquinoxaline sulfonamide (CQS), nimbolide, KB02, ASTX660, lenalidomide, or pomalidomide.

In embodiments, the degradation enhancer is a compound described in US20180050021, WO2016146985, WO2018189554, WO2018119441, WO2018140809, WO2018119448, WO2018119357, WO2018118598, WO2018102067, WO201898280, WO201889736, WO201881530, WO201871606, WO201864589, WO201852949, WO2017223452, WO2017204445, WO2017197055, WO2017197046, WO2017180417, WO2017176958, WO201711371, WO2018226542, WO2018223909, WO2018189554, WO2016169989, WO2016146985, CN105085620B, CN106543185B, U.S. Ser. No. 10/040,804, U.S. Pat. No. 9,938,302, U.S. Ser. No. 10/144,745, U.S. Ser. No. 10/145,848, U.S. Pat. Nos. 9,938,264, 9,632,089, 9,821,068, 9,758,522, 9,500,653, 9,765,019, 8,507,488, 8,299,057, US20180298027, US20180215731, US20170065719, US20170037004, US20160272639, US20150291562, or US20140356322, which are incorporated by reference in their entirety for any purpose.

In embodiments L^{AB} is -L^{AB1}-L^{AB2}-L^{AB3}-L^{AB4}-L^{AB5}-;
L^{AB1}, L^{AB2}, L^{AB3}, L^{AB4}, and L^{AB5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆alkylene, (—O—C₁₋₆alkyl)_{z}-, (—C₁₋₆alkyl-O)_{z}—, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, wherein C₁₋₆alkylene, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, are optionally substituted with one, two, or three R^{20j}; wherein each C₁₋₆alkyl of (—O—C₁₋₆alkyl)_{z}- and (—C₁₋₆alkyl-O)_{z}— is optionally substituted with one, two, or three R^{20j};
is independently an integer from 0 to 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20d}, R^{20e}, R^{20f}, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and

each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl.

In embodiments, L^{AB} is —(O—C₂alkyl)_{z}- and z is an integer from 1 to 10.

In embodiments, L^{AB} is —(C₂alkyl-O—)_{z}— and z is an integer from 1 to 10.

In embodiments, L^{AB} is —(CH₂)_{zz1}L^{AB2}(CH₂O)_{zz2}—, wherein L^{AB2} is a bond, a 5 or 6 membered heterocycloalkylene or heteroarylene, phenylene, —(C₂-C₄)alkynylene, —SO₂— or —NH—; and zz1 and zz2 are independently an integer from 0 to 10.

In embodiments, L^{AB} is —(CH₂)_{zz1}(CH₂O)_{zz2}—, wherein zz1 and zz2 are each independently an integer from 0 to 10.

In embodiments, L^{AB} is a PEG linker (e.g., divalent linker of 1 to 10 ethylene glycol subunits).

In embodiments, R⁴ is independently halogen. In embodiments, R⁴ is independently R⁴ is independently oxo. In embodiments, R⁴ is independently —CN. In embodiments, R⁴ is independently C₁₋₆alkyl. In embodiments, R⁴ is independently C₂₋₆ alkenyl. In embodiments, R⁴ is independently C₂₋₆alkynyl. In embodiments, R⁴ is independently C₁₋₆haloalkyl. In embodiments, R⁴ is independently C₃₋₁₂cycloalkyl. In embodiments, R⁴ is independently —CH₂—C₃₋₁₂cycloalkyl. In embodiments, R⁴ is independently C₁₋₁₁heterocycloalkyl. In embodiments, R⁴ is independently —CH₂—C₁₋₁₁heterocycloalkyl. In embodiments, R⁴ is independently C₆₋₁₂aryl. In embodiments, R⁴ is independently —CH₂—C₆₋₁₂aryl. In embodiments, R⁴ is independently —CH₂—C₁₋₁₁heteroaryl. In embodiments, R⁴ is independently C₁₋₁₁heteroaryl. In embodiments, R⁴ is independently —OR¹². In embodiments, R⁴ is independently —SR¹². In embodiments, R⁴ is independently —N(R¹²)(R¹³). In embodiments, R⁴ is independently —C(O)OR¹². In embodiments, R⁴ is independently —OC(O)N(R¹²)(R¹³). In embodiments, R⁴ is independently —N(R¹⁴)C(O)N(R¹²)(R¹³). In embodiments, R⁴ is independently —N(R¹⁴)C(O)OR¹⁵. In embodiments, R⁴ is independently —N(R¹⁴)S(O)₂R¹⁵. In embodiments, R⁴ is independently —C(O)R¹⁵. In embodiments, R⁴ is independently —S(O)R¹⁵. In embodiments, R⁴ is independently —OC(O)R¹⁵. In embodiments, R⁴ is independently —C(O)N(R¹²)(R¹³). In embodiments, R⁴ is independently —C(O)C(O)N(R¹²)(R¹³). In embodiments, R⁴ is independently —N(R¹⁴)C(O)R¹⁵. In embodiments, R⁴ is independently —S(O)₂R¹⁵. In embodiments, R⁴ is independently —S(O)₂N(R¹²)(R¹³)—. In embodiments, R⁴ is independently S(═O)(═NH)N(R¹²)(R¹³). In embodiments, R⁴ is independently —CH₂C(O)N(R¹²)(R¹³). In embodiments, R⁴ is independently —CH₂N(R¹⁴)C(O)R¹⁵. In embodiments, R⁴ is independently —CH₂S(O)₂R¹⁵. In embodiments, R⁴ is independently and —CH₂S(O)₂N(R¹²)(R¹³).

In embodiments, R⁴ is independently C₁₋₆alkyl optionally substituted with one, two, or three R^{20a}. In embodiments, R⁴ is independently C₂₋₆alkenyl optionally substituted with one, two, or three R^{20a}. In embodiments, R⁴ is independently C₂₋₆alkynyl optionally substituted with one, two, or three R^{20a}. In embodiments, R⁴ is independently C₃₋₆cycloalkyl optionally substituted with one, two, or three R^{20a}. In embodiments, R⁴ is independently C₂₋₉heterocycloalkyl optionally substituted with one, two, or three R^{20a}. In embodiments, R⁴ is independently C₆₋₁₂aryl optionally substituted with one, two, or three R^{20a}. In embodiments, R⁴ is independently C₁₋₉heteroaryl optionally substituted with one, two, or three R^{20a}.

In some embodiments of the compound, or a pharmaceutically acceptable salt or solvate thereof, R¹⁷ is a monocyclic ring. In some embodiments of the compound, or a pharmaceutically acceptable salt or solvate thereof, R¹⁷ is a bicyclic ring system. In some embodiments of the compound, or a pharmaceutically acceptable salt or solvate thereof, R¹⁷ is a polycyclic ring system.

In select embodiments of the compound, R¹⁷ is a C₃₋₁₂cycloalkyl optionally substituted with one, two, three, four, five, six, or seven R¹ⁱ. In additional embodiments of the compound, R¹⁷ is a C₂₋₁₁heterocycloalkyl optionally substituted with one, two, three, four, five, six, or seven R¹ⁱ. In further embodiments of the compound, R¹⁷ is a C₆₋₁₂aryl optionally substituted with one, two, three, four, five, six, or seven R¹ⁱ. In some embodiments of the compound, R¹⁷ is a C₂₋₁₂heteroaryl optionally substituted with one, two, three, four, five, six, or seven R¹ⁱ. In embodiments of the compound, R¹⁷ is a C₃₋₁₂cycloalkyl. In select embodiments of the compound, R¹⁷ is a C₂₋₁₁heterocycloalkyl. In additional embodiments of the compound, R¹⁷ is a C₆₋₁₂aryl. In some embodiments of the compound, R¹⁷ is a C₂₋₁₂heteroaryl. In some embodiments of the compound, or a pharmaceutically acceptable salt or solvate thereof, R¹⁷ is a monocyclic C₃₋₉cycloalkyl optionally substituted with one, two, three, four, five, six, or seven R¹ⁱ. In embodiments of the compound, or a pharmaceutically acceptable salt or solvate thereof, R¹⁷ is a monocyclic C₁₋₅heterocycloalkyl optionally substituted with one, two, three, four, five, six, or seven R¹ⁱ. In additional embodiments of the compound, or a pharmaceutically acceptable salt or solvate thereof, R¹⁷ is a monocyclic phenyl optionally substituted with one, two, three, four, or five R¹ⁱ. In further embodiments of the compound, or a pharmaceutically acceptable salt or solvate thereof, R¹⁷ is a monocyclic C₁₋₅heteroaryl optionally substituted with one, two, three, four, or five R¹ⁱ. In some embodiments of the compound, or a pharmaceutically acceptable salt or solvate thereof, R¹⁷ is a spirocyclic C₅₋₁₂cycloalkyl optionally substituted with one, two, three, four, five, six, or seven R¹ⁱ. In embodiments of the compound, or a pharmaceutically acceptable salt or solvate thereof, R¹⁷ is a spirocyclic C₂₋₁₁heterocycloalkyl optionally substituted with one, two, three, four, five, six, or seven R¹ⁱ. In additional embodiments of the compound, or a pharmaceutically acceptable salt or solvate thereof, R¹⁷ is a fused C₅₋₁₂cycloalkyl optionally substituted with one, two, three, four, five, six, or seven R¹ⁱ. In further embodiments of the compound, or a pharmaceutically acceptable salt or solvate thereof, R¹⁷ is a fused C₂₋₁₁heterocycloalkyl optionally substituted with one, two, three, four, five, six, or seven R¹ⁱ. In further embodiments of the compound, or a pharmaceutically acceptable salt or solvate thereof, R¹⁷ is a fused C₆₋₁₂aryl, optionally substituted with one, two, three, four, five, six, or seven R¹ⁱ. In some embodiments of the compound, or a pharmaceutically acceptable salt or solvate thereof, R¹⁷ is a fused 5 to 12 membered heteroaryl optionally substituted with one, two, three, four, five, six, or seven R¹ⁱ. In embodiments, R¹⁷ is a fused bicyclic C₅₋₁₂cycloalkyl. In embodiments, R¹⁷ is a fused bicyclic C₂₋₁₁heterocycloalkyl. In embodiments, R¹⁷ is a fused bicyclic C₇₋₁₂aryl. In embodiments, R¹⁷ is a fused bicyclic C₂₋₁₂heteroaryl. In embodiments, R¹⁷ is a fused bicyclic C₅₋₁₂cycloalkyl substituted with one, two, three, four, five, six, or seven R¹ⁱ. In embodiments, R¹⁷ is a fused bicyclic C₂₋₁₁heterocycloalkyl substituted with one, two, three, four, five, six, or seven R¹ⁱ. In embodiments, R¹⁷ is a fused bicyclic C₇₋₁₂aryl substituted with one, two, three, four, five, six, or seven R¹ⁱ. In embodiments, R¹⁷ is a fused bicyclic C₂₋₁₂heteroaryl substituted with one, two, three, four, five, six, or seven R¹ⁱ. In embodiments, R¹⁷ is a fused bicyclic C₅₋₁₂cycloalkyl optionally substituted with one, two, three, four, five, six, or seven R¹ⁱ. In embodiments, R¹⁷ is a fused bicyclic C₂₋₉heterocycloalkyl optionally substituted with one, two, three, four, five, six, or seven R¹ⁱ. In embodiments, R¹⁷ is a fused bicyclic C₇₋₁₂aryl optionally substituted with one, two, three, four, five, six, or seven R¹ⁱ. In embodiments, R¹⁷ is a fused bicyclic C₂₋₁₂heteroaryl optionally substituted with one, two, three, four, five, six, or seven R¹ⁱ. In embodiments, R¹⁷ is a C₃₋₁₂cycloalkyl. In embodiments, R¹⁷ is a C₂₋₁₁heterocycloalkyl. In embodiments, R¹⁷ is a C₆₋₁₂aryl. In embodiments, R¹⁷ is a C₂₋₁₂heteroaryl. In embodiments, R¹⁷ is a C₃₋₁₂cycloalkyl optionally substituted with one, two, three, four, five, six, or seven R¹ⁱ. In embodiments, R¹⁷ is a C₂₋₁₁heterocycloalkyl optionally substituted with one, two, three, four, five, six, or seven R¹ⁱ. In embodiments, R¹⁷ is a C₆₋₁₂aryl optionally substituted with one, two, three, four, five, six, or seven R¹ⁱ. In embodiments, R¹⁷ is a C₂₋₁₂heteroaryl optionally substituted with one, two, three, four, five, six, or seven R¹ⁱ. In embodiments, R¹⁷ is a C₃₋₁₂cycloalkyl substituted with one, two, three, four, five, six, or seven R¹ⁱ. In embodiments, R¹⁷ is a C₂₋₁₁heterocycloalkyl substituted with one, two, three, four, five, six, or seven R¹ⁱ. In embodiments, R¹⁷ is a C₆₋₁₂aryl substituted with one, two, three, four, five, six, or seven R¹ⁱ. In embodiments, R¹⁷ is a C₂₋₁₂heteroaryl substituted with one, two, three, four, five, six, or seven R¹ⁱ.

In embodiments, R¹ⁱ is independently hydrogen. In embodiments, R¹ⁱ is independently halogen. In embodiments, R¹ⁱ is independently oxo. In embodiments, R¹ⁱ is independently —CN. In embodiments, R¹ⁱ is independently C₁₋₆alkyl. In embodiments, R¹ⁱ is independently C₂₋₆alkenyl. In embodiments, R¹ⁱ is independently C₂₋₆alkynyl. In embodiments, R¹ⁱ is independently C₃₋₁₀cycloalkyl. In embodiments, R¹ⁱ is independently C₂₋₉heterocycloalkyl. In embodiments, R¹ⁱ is independently C₆₋₁₀aryl. In embodiments, R¹ⁱ is independently C₁₋₉heteroaryl.

In embodiments, R¹ⁱ is independently C₁₋₆alkyl optionally substituted with one, two, or three R²⁰ⁱ. In embodiments, R¹ⁱ is independently C₂₋₆alkenyl optionally substituted with one, two, or three R²⁰ⁱ. In embodiments, R¹ⁱ is independently C₂₋₆alkynyl optionally substituted with one, two, or three R²⁰ⁱ. In embodiments, R¹ⁱ is independently C₃₋₁₀cycloalkyl optionally substituted with one, two, or three R²⁰ⁱ. In embodiments, R¹ⁱ is independently C₂₋₉heterocycloalkyl optionally substituted with one, two, or three R²⁰ⁱ. In embodiments, R¹ⁱ is independently C₆₋₁₀aryl optionally substituted with one, two, or three R²⁰ⁱ. In embodiments, R¹ⁱ is independently C₁₋₉heteroaryl optionally substituted with one, two, or three R²⁰ⁱ.

In embodiments, R¹ⁱ is independently —OR¹². In embodiments, R¹ⁱ is independently —SR¹². In embodiments, R¹ⁱ is independently —N(R¹²)(R¹³). In embodiments, R¹ⁱ is independently —C(O)OR¹². In embodiments, R¹ⁱ is independently —OC(O)N(R¹²)(R¹³). In embodiments, R¹ⁱ is independently —N(R¹⁴)C(O)N(R¹²)(R¹³). In embodiments, R¹ⁱ is independently —N(R¹⁴)C(O)OR¹⁵. In embodiments, R¹ⁱ is independently —N(R¹⁴)S(O)₂R¹⁵. In embodiments, R¹ⁱ is independently —C(O)R¹⁵. In embodiments, R¹ⁱ is independently —S(O)R¹⁵. In embodiments, R¹ⁱ is independently —OC(O)R¹⁵. In embodiments, R¹ⁱ is independently —C(O)N(R¹²)(R¹³). In embodiments, R¹ⁱ is independently —C(O)C(O)N(R¹²)(R¹³). In embodiments, R¹ⁱ is independently —N(R¹⁴)C(O)R¹⁵. In embodiments, R¹ⁱ is independently —S(O)₂R¹⁵. In embodiments, R¹ⁱ is independently —S(O)₂N(R¹²)(R¹³). In embodiments, R¹ⁱ is independently S(═O)(═NH)N(R¹²)(R¹³). In embodiments, R¹ⁱ is independently —CH₂C(O)N(R¹²)(R¹³). In embodiments, R¹ⁱ is independently —CH₂N(R¹⁴)C(O)R¹⁵. In embodiments, R¹ⁱ is independently —CH₂S(O)₂R¹⁵. In embodiments, R¹ⁱ is independently —CH₂S(O)₂N(R¹²)(R¹³).

In some embodiments of the compound, or a pharmaceutically acceptable salt or solvate thereof, R¹⁹ is a monocyclic ring. In some embodiments of the compound, or a pharmaceutically acceptable salt or solvate thereof, R¹⁹ is a bicyclic ring system. In some embodiments of the compound, or a pharmaceutically acceptable salt or solvate thereof, R¹⁹ is a polycyclic ring system.

In select embodiments of the compound, R¹⁹ is a C₃₋₁₂cycloalkyl optionally substituted with one, two, three, four, five, six, or seven R¹ⁱ. In additional embodiments of the compound, R¹⁹ is a C₂₋₁₁heterocycloalkyl optionally substituted with one, two, three, four, five, six, or seven R¹ⁱ. In further embodiments of the compound, R¹⁹ is a C₆₋₁₂aryl optionally substituted with one, two, three, four, five, six, or seven R¹ⁱ. In some embodiments of the compound, R¹⁹ is a C₂₋₁₂heteroaryl optionally substituted with one, two, three, four, five, six, or seven R¹ⁱ. In embodiments of the compound, R¹⁹ is a C₃₋₁₂cycloalkyl. In select embodiments of the compound, R¹⁹ is a C₂₋₁₁heterocycloalkyl. In additional embodiments of the compound, R¹⁹ is a C₆₋₁₂aryl. In some embodiments of the compound, R¹⁹ is a C₂₋₁₂heteroaryl. In some embodiments of the compound, or a pharmaceutically acceptable salt or solvate thereof, R¹⁹ is a monocyclic C₃₋₉cycloalkyl optionally substituted with one, two, three, four, five, six, or seven R¹ⁱ. In embodiments of the compound, or a pharmaceutically acceptable salt or solvate thereof, R¹⁹ is a monocyclic C₁₋₅heterocycloalkyl optionally substituted with one, two, three, four, five, six, or seven R¹ⁱ. In additional embodiments of the compound, or a pharmaceutically acceptable salt or solvate thereof, R¹⁹ is a monocyclic phenyl optionally substituted with one, two, three, four, or five R¹ⁱ. In further embodiments of the compound, or a pharmaceutically acceptable salt or solvate thereof, R¹⁹ is a monocyclic C₁₋₅heteroaryl optionally substituted with one, two, three, four, or five R¹ⁱ. In some embodiments of the compound, or a pharmaceutically acceptable salt or solvate thereof, R¹⁹ is a spirocyclic C₅₋₁₂cycloalkyl optionally substituted with one, two, three, four, five, six, or seven R¹ⁱ. In embodiments of the compound, or a pharmaceutically acceptable salt or solvate thereof, R¹⁹ is a spirocyclic C₂₋₁₁heterocycloalkyl optionally substituted with one, two, three, four, five, six, or seven R¹ⁱ. In additional embodiments of the compound, or a pharmaceutically acceptable salt or solvate thereof, R¹⁹ is a fused C₅₋₁₂cycloalkyl optionally substituted with one, two, three, four, five, six, or seven R¹ⁱ. In further embodiments of the compound, or a pharmaceutically acceptable salt or solvate thereof, R¹⁹ is a fused C₂₋₁₁heterocycloalkyl optionally substituted with one, two, three, four, five, six, or seven R¹ⁱ. In further embodiments of the compound, or a pharmaceutically acceptable salt or solvate thereof, R¹⁹ is a fused C₆₋₁₂aryl, optionally substituted with one, two, three, four, five, six, or seven R¹ⁱ. In some embodiments of the compound, or a pharmaceutically acceptable salt or solvate thereof, R¹⁹ is a fused 5 to 12 membered heteroaryl optionally substituted with one, two, three, four, five, six, or seven R¹ⁱ. In embodiments, R¹⁹ is a fused bicyclic C₅₋₁₂cycloalkyl. In embodiments, R¹⁹ is a fused bicyclic C₂₋₁₁heterocycloalkyl. In embodiments, R¹⁹ is a fused bicyclic C₇₋₁₂aryl. In embodiments, R¹⁹ is a fused bicyclic C₂₋₁₂heteroaryl. In embodiments, R¹⁹ is a fused bicyclic C₅₋₁₂cycloalkyl substituted with one, two, three, four, five, six, or seven R¹ⁱ. In embodiments, R¹⁹ is a fused bicyclic C₂₋₁₁heterocycloalkyl substituted with one, two, three, four, five, six, or seven R¹ⁱ. In embodiments, R¹⁹ is a fused bicyclic C₇₋₁₂aryl substituted with one, two, three, four, five, six, or seven R¹ⁱ. In embodiments, R¹⁹ is a fused bicyclic C₂₋₁₂heteroaryl substituted with one, two, three, four, five, six, or seven R¹ⁱ. In embodiments, R¹⁹ is a fused bicyclic C₅₋₁₂cycloalkyl optionally substituted with one, two, three, four, five, six, or seven R¹ⁱ. In embodiments, R¹⁹ is a fused bicyclic C₂₋₁₁heterocycloalkyl optionally substituted with one, two, three, four, five, six, or seven R¹ⁱ. In embodiments, R¹⁹ is a fused bicyclic C₇₋₁₂aryl optionally substituted with one, two, three, four, five, six, or seven R¹ⁱ. In embodiments, R¹⁹ is a fused bicyclic C₂₋₁₂heteroaryl optionally substituted with one, two, three, four, five, six, or seven R¹ⁱ. In embodiments, R¹⁹ is a C₃₋₁₂cycloalkyl. In embodiments, R¹⁹ is a C₂₋₁₁heterocycloalkyl. In embodiments, R¹⁹ is a C₆₋₁₂aryl. In embodiments, R¹⁹ is a C₂₋₁₂heteroaryl. In embodiments, R¹⁹ is a C₃₋₁₂cycloalkyl optionally substituted with one, two, three, four, five, six, or seven R¹ⁱ. In embodiments, R¹⁹ is a C₂₋₁₁heterocycloalkyl optionally substituted with one, two, three, four, five, six, or seven R¹ⁱ. In embodiments, R¹⁹ is a C₆₋₁₂aryl optionally substituted with one, two, three, four, five, six, or seven R¹ⁱ. In embodiments, R¹⁹ is a C₂₋₁₂heteroaryl optionally substituted with one, two, three, four, five, six, or seven R¹ⁱ. In embodiments, R¹⁹ is a C₃₋₁₂cycloalkyl substituted with one, two, three, four, five, six, or seven R¹ⁱ. In embodiments, R¹⁹ is a C₂₋₁₁heterocycloalkyl substituted with one, two, three, four, five, six, or seven R¹ⁱ. In embodiments, R¹⁹ is a C₆₋₁₂aryl substituted with one, two, three, four, five, six, or seven R¹ⁱ. In embodiments, R¹⁹ is a C₂₋₁₂heteroaryl substituted with one, two, three, four, five, six, or seven R¹ⁱ.

In embodiments, R¹ⁱ is independently hydrogen. In embodiments, R¹ⁱ is independently halogen. In embodiments, R¹ⁱ is independently oxo. In embodiments, R¹ⁱ is independently —CN. In embodiments, R¹ⁱ is independently C₁₋₆alkyl. In embodiments, R¹ⁱ is independently C₂₋₆alkenyl. In embodiments, R¹ⁱ is independently C₂₋₆alkynyl. In embodiments, R¹ⁱ is independently C₃₋₁₀cycloalkyl. In embodiments, R¹ⁱ is independently C₂₋₉heterocycloalkyl. In embodiments, R¹ⁱ is independently C₆₋₁₀aryl. In embodiments, R¹ⁱ is independently C₁₋₉heteroaryl.

In embodiments, R¹ⁱ is independently C₁₋₆alkyl optionally substituted with one, two, or three R²⁰ⁱ. In embodiments, R¹ⁱ is independently C₂₋₆alkenyl optionally substituted with one, two, or three R²⁰ⁱ. In embodiments, R¹ⁱ is independently C₂₋₆alkynyl optionally substituted with one, two, or three R²⁰ⁱ. In embodiments, R¹ⁱ is independently C₃₋₁₀cycloalkyl optionally substituted with one, two, or three R²⁰ⁱ. In embodiments, R¹ⁱ is independently C₂₋₉heterocycloalkyl optionally substituted with one, two, or three R²⁰ⁱ. In embodiments, R¹ⁱ is independently C₆₋₁₀aryl optionally substituted with one, two, or three R²⁰ⁱ. In embodiments, R¹ⁱ is independently C₁₋₉heteroaryl optionally substituted with one, two, or three R²⁰ⁱ.

In embodiments, R¹ⁱ is independently —OR¹². In embodiments, R¹ⁱ is independently —SR¹². In embodiments, R¹ⁱ is independently —N(R¹²)(R¹³). In embodiments, R¹ⁱ is independently —C(O)OR¹². In embodiments, R¹ⁱ is independently —OC(O)N(R¹²)(R¹³). In embodiments, R¹ⁱ is independently —N(R¹⁴)C(O)N(R¹²)(R¹³). In embodiments, R¹ⁱ is independently —N(R¹⁴)C(O)OR¹⁵. In embodiments, R¹ⁱ is independently —N(R¹⁴)S(O)₂R¹⁵. In embodiments, R¹ⁱ is independently —C(O)R¹⁵. In embodiments, R¹ⁱ is independently —S(O)R¹⁵. In embodiments, R¹ⁱ is independently —OC(O)R¹⁵. In embodiments, R¹ⁱ is independently —C(O)N(R¹²)(R¹³). In embodiments, R¹ⁱ is independently —C(O)C(O)N(R¹²)(R¹³). In embodiments, R¹ⁱ is independently —N(R¹⁴)C(O)R¹⁵. In embodiments, R¹ⁱ is independently —S(O)₂R¹⁵. In embodiments, R¹ⁱ is independently —S(O)₂N(R¹²)(R¹³). In embodiments, R¹ⁱ is independently S(═O)(═NH)N(R¹²)(R¹³). In embodiments, R¹ⁱ is independently —CH₂C(O)N(R¹²)(R¹³). In embodiments, R¹ⁱ is independently —CH₂N(R¹⁴)C(O)R¹⁵. In embodiments, R¹ⁱ is independently —CH₂S(O)₂R¹⁵. In embodiments, R¹ⁱ is independently —CH₂S(O)₂N(R¹²)(R¹³).

In some embodiments of the compound, or a pharmaceutically acceptable salt or solvate thereof, R¹⁷ is a monocyclic ring. In some embodiments of the compound, or a pharmaceutically acceptable salt or solvate thereof, R¹⁷ is a bicyclic ring system. In some embodiments of the compound, or a pharmaceutically acceptable salt or solvate thereof, R¹⁷ is a polycyclic ring system.

In select embodiments of the compound, R¹⁷ is a C₃₋₁₂cycloalkyl optionally substituted with one, two, three, four, five, six, or seven R²⁰ⁱ. In additional embodiments of the compound, R¹⁷ is a C₂₋₁₁heterocycloalkyl optionally substituted with one, two, three, four, five, six, or seven R²⁰ⁱ. In further embodiments of the compound, R¹⁷ is a C₆₋₁₂aryl optionally substituted with one, two, three, four, five, six, or seven R²⁰ⁱ. In some embodiments of the compound, R¹⁷ is a C₂₋₁₂heteroaryl optionally substituted with one, two, three, four, five, six, or seven R²⁰ⁱ. In embodiments of the compound, R¹⁷ is a C₃₋₁₂cycloalkyl. In select embodiments of the compound, R¹⁷ is a C₂₋₁₁heterocycloalkyl. In additional embodiments of the compound, R¹⁷ is a C₆₋₁₂aryl. In some embodiments of the compound, R¹⁷ is a C₂₋₁₂heteroaryl. In some embodiments of the compound, or a pharmaceutically acceptable salt or solvate thereof, R¹⁷ is a monocyclic C₃₋₉cycloalkyl optionally substituted with one, two, three, four, five, six, or seven R²⁰ⁱ. In embodiments of the compound, or a pharmaceutically acceptable salt or solvate thereof, R¹⁷ is a monocyclic C₁₋₅heterocycloalkyl optionally substituted with one, two, three, four, five, six, or seven R²⁰ⁱ. In additional embodiments of the compound, or a pharmaceutically acceptable salt or solvate thereof, R¹⁷ is a monocyclic phenyl optionally substituted with one, two, three, four, or five R²⁰ⁱ. In further embodiments of the compound, or a pharmaceutically acceptable salt or solvate thereof, R¹⁷ is a monocyclic C₁₋₅heteroaryl optionally substituted with one, two, three, four, or five R²⁰ⁱ. In some embodiments of the compound, or a pharmaceutically acceptable salt or solvate thereof, R¹⁷ is a spirocyclic C₅₋₁₂cycloalkyl optionally substituted with one, two, three, four, five, six, or seven R²⁰ⁱ. In embodiments of the compound, or a pharmaceutically acceptable salt or solvate thereof, R¹⁷ is a spirocyclic C₂₋₁₁heterocycloalkyl optionally substituted with one, two, three, four, five, six, or seven R²⁰ⁱ. In additional embodiments of the compound, or a pharmaceutically acceptable salt or solvate thereof, R¹⁷ is a fused C₅₋₁₂cycloalkyl optionally substituted with one, two, three, four, five, six, or seven R²⁰ⁱ. In further embodiments of the compound, or a pharmaceutically acceptable salt or solvate thereof, R¹⁷ is a fused C₂₋₁₁heterocycloalkyl optionally substituted with one, two, three, four, five, six, or seven R²⁰ⁱ. In further embodiments of the compound, or a pharmaceutically acceptable salt or solvate thereof, R¹⁷ is a fused C₆₋₁₂aryl, optionally substituted with one, two, three, four, five, six, or seven R²⁰ⁱ. In some embodiments of the compound, or a pharmaceutically acceptable salt or solvate thereof, R¹⁷ is a fused 5 to 12 membered heteroaryl optionally substituted with one, two, three, four, five, six, or seven R²⁰ⁱ. In embodiments, R¹⁷ is a fused bicyclic C₅₋₁₂cycloalkyl. In embodiments, R¹⁷ is a fused bicyclic C₂₋₁₁heterocycloalkyl. In embodiments, R¹⁷ is a fused bicyclic C₇₋₁₂aryl. In embodiments, R¹⁷ is a fused bicyclic C₂₋₁₂heteroaryl. In embodiments, R¹⁷ is a fused bicyclic C₅₋₁₂cycloalkyl substituted with one, two, three, four, five, six, or seven R²⁰ⁱ. In embodiments, R¹⁷ is a fused bicyclic C₂₋₁₁heterocycloalkyl substituted with one, two, three, four, five, six, or seven R²⁰ⁱ. In embodiments, R¹⁷ is a fused bicyclic C₇₋₁₂aryl substituted with one, two, three, four, five, six, or seven R²⁰ⁱ. In embodiments, R¹⁷ is a fused bicyclic C₂₋₁₂heteroaryl substituted with one, two, three, four, five, six, or seven R²⁰ⁱ. In embodiments, R¹⁷ is a fused bicyclic C₅₋₁₂cycloalkyl optionally substituted with one, two, three, four, five, six, or seven R²⁰ⁱ. In embodiments, R¹⁷ is a fused bicyclic C₂₋₁₁heterocycloalkyl optionally substituted with one, two, three, four, five, six, or seven R²⁰ⁱ. In embodiments, R¹⁷ is a fused bicyclic C₇₋₁₂aryl optionally substituted with one, two, three, four, five, six, or seven R²⁰ⁱ. In embodiments, R¹⁷ is a fused bicyclic C₂₋₁₂heteroaryl optionally substituted with one, two, three, four, five, six, or seven R²⁰ⁱ. In embodiments, R¹⁷ is a C₃₋₁₂cycloalkyl. In embodiments, R¹⁷ is a C₂₋₁₁heterocycloalkyl. In embodiments, R¹⁷ is a C₆₋₁₂aryl. In embodiments, R¹⁷ is a C₂₋₁₂heteroaryl. In embodiments, R¹⁷ is a C₃₋₁₂cycloalkyl optionally substituted with one, two, three, four, five, six, or seven R²⁰ⁱ. In embodiments, R¹⁷ is a C₂₋₁₁heterocycloalkyl optionally substituted with one, two, three, four, five, six, or seven R²⁰ⁱ. In embodiments, R¹⁷ is a C₆₋₁₂aryl optionally substituted with one, two, three, four, five, six, or seven R²⁰ⁱ. In embodiments, R¹⁷ is a C₂₋₁₂heteroaryl optionally substituted with one, two, three, four, five, six, or seven R²⁰ⁱ. In embodiments, R¹⁷ is a C₃₋₁₂cycloalkyl substituted with one, two, three, four, five, six, or seven R²⁰ⁱ. In embodiments, R¹⁷ is a C₂₋₁₁heterocycloalkyl substituted with one, two, three, four, five, six, or seven R²⁰ⁱ. In embodiments, R¹⁷ is a C₆₋₁₂aryl substituted with one, two, three, four, five, six, or seven R²⁰ⁱ. In embodiments, R¹⁷ is a C₂₋₁₂heteroaryl substituted with one, two, three, four, five, six, or seven R²⁰ⁱ.

In embodiments, R²⁰ⁱ is independently hydrogen. In embodiments, R²⁰ⁱ is independently halogen. In embodiments, R²⁰ⁱ is independently oxo. In embodiments, R²⁰ⁱ is independently —CN. In embodiments, R²⁰ⁱ is independently C₁₋₆alkyl. In embodiments, R²⁰ⁱ is independently C₂₋₆alkenyl. In embodiments, R²⁰ⁱ is independently C₂₋₆alkynyl. In embodiments, R²⁰ⁱ is independently C₃₋₁₀cycloalkyl. In embodiments, R²⁰ⁱ is independently C₂₋₉heterocycloalkyl. In embodiments, R²⁰ⁱ is independently C₆₋₁₀aryl. In embodiments, R²⁰ⁱ is independently C₁₋₉heteroaryl.

In embodiments, R²⁰ⁱ is independently —OR¹². In embodiments, R²⁰ⁱ is independently —SR¹². In embodiments, R²⁰ⁱ is independently —N(R¹²)(R¹³). In embodiments, R²⁰ⁱ is independently —C(O)OR¹². In embodiments, R²⁰ⁱ is independently —OC(O)N(R¹²)(R¹³). In embodiments, R²⁰ⁱ is independently —N(R¹⁴)C(O)N(R¹²)(R¹³). In embodiments, R²⁰ⁱ is independently —N(R¹⁴)C(O)OR¹⁵. In embodiments, R²⁰ⁱ is independently —N(R¹⁴)S(O)₂R¹⁵. In embodiments, R²⁰ⁱ is independently —C(O)R¹⁵. In embodiments, R²⁰ⁱ is independently —S(O)R¹⁵. In embodiments, R²⁰ⁱ is independently —OC(O)R¹⁵. In embodiments, R²⁰ⁱ is independently —C(O)N(R¹²)(R¹³). In embodiments, R²⁰ⁱ is independently —C(O)C(O)N(R¹²)(R¹³). In embodiments, R²⁰ⁱ is independently —N(R¹⁴)C(O)R¹⁵. In embodiments, R²⁰ⁱ is independently —S(O)₂R¹⁵. In embodiments, R²⁰ⁱ is independently —S(O)₂N(R¹²)(R¹³). In embodiments, R²⁰ⁱ is independently S(═O)(═NH)N(R¹²)(R¹³). In embodiments, R²⁰ⁱ is independently —CH₂C(O)N(R¹²)(R¹³). In embodiments, R²⁰ⁱ is independently —CH₂N(R¹⁴)C(O)R¹⁵. In embodiments, R²⁰ⁱ is independently —CH₂S(O)₂R¹⁵. In embodiments, R²⁰ⁱ is independently —CH₂S(O)₂N(R¹²)(R¹³).

In some embodiments of the compound, or a pharmaceutically acceptable salt or solvate thereof, R¹⁹ is independently a monocyclic ring. In some embodiments of the compound, or a pharmaceutically acceptable salt or solvate thereof, R¹⁹ is independently a bicyclic ring system. In some embodiments of the compound, or a pharmaceutically acceptable salt or solvate thereof, R¹⁹ is independently a polycyclic ring system.

In select embodiments of the compound, R¹⁹ is a C₃₋₁₂cycloalkyl optionally substituted with one, two, three, four, five, six, or seven R²⁰ⁱ. In additional embodiments of the compound, R¹⁹ is a C₂₋₁₁heterocycloalkyl optionally substituted with one, two, three, four, five, six, or seven R²⁰ⁱ. In further embodiments of the compound, R¹⁹ is a C₆₋₁₂aryl optionally substituted with one, two, three, four, five, six, or seven R²⁰ⁱ. In some embodiments of the compound, R¹⁹ is a C₂₋₁₂heteroaryl optionally substituted with one, two, three, four, five, six, or seven R²⁰ⁱ. In embodiments of the compound, R¹⁹ is a C₃₋₁₂cycloalkyl. In select embodiments of the compound, R¹⁹ is a C₂₋₁₁heterocycloalkyl. In additional embodiments of the compound, R¹⁹ is a C₆₋₁₂aryl. In some embodiments of the compound, R¹⁹ is a C₂₋₁₂heteroaryl. In some embodiments of the compound, or a pharmaceutically acceptable salt or solvate thereof, R¹⁹ is a monocyclic C₃₋₉cycloalkyl optionally substituted with one, two, three, four, five, six, or seven R²⁰ⁱ. In embodiments of the compound, or a pharmaceutically acceptable salt or solvate thereof, R¹⁹ is a monocyclic C₁₋₅heterocycloalkyl optionally substituted with one, two, three, four, five, six, or seven R²⁰ⁱ. In additional embodiments of the compound, or a pharmaceutically acceptable salt or solvate thereof, R¹⁹ is a monocyclic phenyl optionally substituted with one, two, three, four, or five R²⁰ⁱ. In further embodiments of the compound, or a pharmaceutically acceptable salt or solvate thereof, R¹⁹ is a monocyclic C₁₋₅heteroaryl optionally substituted with one, two, three, four, or five R²⁰ⁱ. In some embodiments of the compound, or a pharmaceutically acceptable salt or solvate thereof, R¹⁹ is a spirocyclic C₅₋₁₂cycloalkyl optionally substituted with one, two, three, four, five, six, or seven R²⁰ⁱ. In embodiments of the compound, or a pharmaceutically acceptable salt or solvate thereof, R¹⁹ is a spirocyclic C₂₋₁₁heterocycloalkyl optionally substituted with one, two, three, four, five, six, or seven R²⁰ⁱ. In additional embodiments of the compound, or a pharmaceutically acceptable salt or solvate thereof, R¹⁹ is a fused C₅₋₁₂cycloalkyl optionally substituted with one, two, three, four, five, six, or seven R²⁰ⁱ. In further embodiments of the compound, or a pharmaceutically acceptable salt or solvate thereof, R¹⁹ is a fused C₂₋₁₁heterocycloalkyl optionally substituted with one, two, three, four, five, six, or seven R²⁰ⁱ. In further embodiments of the compound, or a pharmaceutically acceptable salt or solvate thereof, R¹⁹ is a fused C₆₋₁₂aryl, optionally substituted with one, two, three, four, five, six, or seven R²⁰ⁱ. In some embodiments of the compound, or a pharmaceutically acceptable salt or solvate thereof, R¹⁹ is a fused 5 to 12 membered heteroaryl optionally substituted with one, two, three, four, five, six, or seven R²⁰ⁱ. In embodiments, R¹⁹ is a fused bicyclic C₅₋₁₂cycloalkyl. In embodiments, R¹⁹ is a fused bicyclic C₂₋₁₁heterocycloalkyl. In embodiments, R¹⁹ is a fused bicyclic C₇₋₁₂aryl. In embodiments, R¹⁹ is a fused bicyclic C₂₋₁₂heteroaryl. In embodiments, R¹⁹ is a fused bicyclic C₅₋₁₂cycloalkyl substituted with one, two, three, four, five, six, or seven R²⁰ⁱ. In embodiments, R¹⁹ is a fused bicyclic C₂₋₁₁heterocycloalkyl substituted with one, two, three, four, five, six, or seven R²⁰ⁱ. In embodiments, R¹⁹ is a fused bicyclic C₇₋₁₂aryl substituted with one, two, three, four, five, six, or seven R²⁰ⁱ. In embodiments, R¹⁹ is a fused bicyclic C₂₋₁₂heteroaryl substituted with one, two, three, four, five, six, or seven R²⁰ⁱ. In embodiments, R¹⁹ is a fused bicyclic C₅₋₁₂cycloalkyl optionally substituted with one, two, three, four, five, six, or seven R²⁰ⁱ. In embodiments, R¹⁹ is a fused bicyclic C₂₋₁₁heterocycloalkyl optionally substituted with one, two, three, four, five, six, or seven R²⁰ⁱ. In embodiments, R¹⁹ is a fused bicyclic C₇₋₁₂aryl optionally substituted with one, two, three, four, five, six, or seven R²⁰ⁱ. In embodiments, R¹⁹ is a fused bicyclic C₂₋₁₂heteroaryl optionally substituted with one, two, three, four, five, six, or seven R²⁰ⁱ. In embodiments, R¹⁹ is a C₃₋₁₂cycloalkyl. In embodiments, R¹⁹ is a C₂₋₁₁heterocycloalkyl. In embodiments, R¹⁹ is a C₆₋₁₂aryl. In embodiments, R¹⁹ is a C₂₋₁₂heteroaryl. In embodiments, R¹⁹ is a C₃₋₁₂cycloalkyl optionally substituted with one, two, three, four, five, six, or seven R²⁰ⁱ. In embodiments, R¹⁹ is a C₂₋₁₁heterocycloalkyl optionally substituted with one, two, three, four, five, six, or seven R²⁰ⁱ. In embodiments, R¹⁹ is a C₆₋₁₂aryl optionally substituted with one, two, three, four, five, six, or seven R²⁰ⁱ. In embodiments, R¹⁹ is a C₂₋₁₂heteroaryl optionally substituted with one, two, three, four, five, six, or seven R²⁰ⁱ. In embodiments, R¹⁹ is a C₃₋₁₂cycloalkyl substituted with one, two, three, four, five, six, or seven R²⁰ⁱ. In embodiments, R¹⁹ is a C₂₋₁₁heterocycloalkyl substituted with one, two, three, four, five, six, or seven R²⁰ⁱ. In embodiments, R¹⁹ is a C₆₋₁₂aryl substituted with one, two, three, four, five, six, or seven R²⁰ⁱ. In embodiments, R¹⁹ is a C₂₋₁₂heteroaryl substituted with one, two, three, four, five, six, or seven R²⁰ⁱ.

In embodiments, R²⁰ⁱ is independently hydrogen. In embodiments, R²⁰ⁱ is independently halogen. In embodiments, R²⁰ⁱ is independently oxo. In embodiments, R²⁰ⁱ is independently —CN. In embodiments, R²⁰ⁱ is independently C₁₋₆alkyl. In embodiments, R²⁰ⁱ is independently C₂₋₆alkenyl. In embodiments, R²⁰ⁱ is independently C₂₋₆alkynyl. In embodiments, R²⁰ⁱ is independently C₃₋₁₀cycloalkyl. In embodiments, R²⁰ⁱ is independently C₂₋₉heterocycloalkyl. In embodiments, R²⁰ⁱ is independently C₆₋₁₀aryl. In embodiments, R²⁰ⁱ is independently C₁₋₉heteroaryl.

In embodiments, R²⁰ⁱ is independently —OR¹². In embodiments, R²⁰ⁱ is independently —SR¹². In embodiments, R²⁰ⁱ is independently —N(R¹²)(R¹³). In embodiments, R²⁰ⁱ is independently —C(O)OR¹². In embodiments, R²⁰ⁱ is independently —OC(O)N(R¹²)(R¹³). In embodiments, R²⁰ⁱ is independently —N(R¹⁴)C(O)N(R¹²)(R¹³). In embodiments, R²⁰ⁱ is independently —N(R¹⁴)C(O)OR¹⁵. In embodiments, R²⁰ⁱ is independently —N(R¹⁴)S(O)₂R¹⁵. In embodiments, R²⁰ⁱ is independently —C(O)R¹⁵. In embodiments, R²⁰ⁱ is independently —S(O)R¹⁵. In embodiments, R²⁰ⁱ is independently —OC(O)R¹⁵. In embodiments, R²⁰ⁱ is independently —C(O)N(R¹²)(R¹³). In embodiments, R²⁰ⁱ is independently —C(O)C(O)N(R¹²)(R¹³). In embodiments, R²⁰ⁱ is independently —N(R¹⁴)C(O)R¹⁵. In embodiments, R²⁰ⁱ is independently —S(O)₂R¹⁵. In embodiments, R²⁰ⁱ is independently —S(O)₂N(R¹²)(R¹³). In embodiments, R²⁰ⁱ is independently S(═O)(═NH)N(R¹²)(R¹³). In embodiments, R²⁰ⁱ is independently —CH₂C(O)N(R¹²)(R¹³). In embodiments, R²⁰ⁱ is independently —CH₂N(R¹⁴)C(O)R¹⁵. In embodiments, R²⁰ⁱ is independently —CH₂S(O)₂R¹⁵. In embodiments, R²⁰ⁱ is independently —CH₂S(O)₂N(R¹²)(R¹³).

In some embodiments of the subject compound, or a pharmaceutically acceptable salt or solvate thereof, R¹ⁱ is independently halogen. In some embodiments of the subject compound, or a pharmaceutically acceptable salt or solvate thereof, R¹ⁱ is independently —CN. In some embodiments of the subject compound, or a pharmaceutically acceptable salt or solvate thereof, R¹ⁱ is independently C₁₋₆alkyl optionally substituted with one, two, or three R²⁰ⁱ. In some embodiments of the subject compound, or a pharmaceutically acceptable salt or solvate thereof, R¹ⁱ is independently C₁₋₆haloalkyl. In some embodiments of the subject compound, or a pharmaceutically acceptable salt or solvate thereof, R¹ⁱ is independently —OR¹². In some embodiments of the subject compound, or a pharmaceutically acceptable salt or solvate thereof, R¹ⁱ is independently —SR¹². In some embodiments of the subject compound, or a pharmaceutically acceptable salt or solvate thereof, R¹ⁱ is independently —N(R¹²)(R¹³). In some embodiments of the subject compound, or a pharmaceutically acceptable salt or solvate thereof, R¹ⁱ is independently —C(O)OR¹². In some embodiments of the subject compound, or a pharmaceutically acceptable salt or solvate thereof, R¹ⁱ is independently —C(O)N(R¹²)(R¹³). In some embodiments of the subject compound, or a pharmaceutically acceptable salt or solvate thereof, R¹ⁱ is independently —OH. In some embodiments of the subject compound, or a pharmaceutically acceptable salt or solvate thereof, R¹ⁱ is independently —SH. In some embodiments of the subject compound, or a pharmaceutically acceptable salt or solvate thereof, R¹ⁱ is independently —NH₂. In some embodiments of the subject compound, or a pharmaceutically acceptable salt or solvate thereof, R¹ⁱ is independently —C(O)OH. In some embodiments of the subject compound, or a pharmaceutically acceptable salt or solvate thereof, R¹ⁱ is independently —C(O)NH₂.

In some embodiments of the subject compound, or a pharmaceutically acceptable salt or solvate thereof, Q¹, Q³, and Q⁵ are independently N or C(R^{1d}). In some embodiments of the subject compound, or a pharmaceutically acceptable salt or solvate thereof, Q¹ is independently N. In some embodiments of the subject compound, or a pharmaceutically acceptable salt or solvate thereof, Q¹ is independently C(R^{1d}). In some embodiments of the subject compound, or a pharmaceutically acceptable salt or solvate thereof, Q³ is independently N. In some embodiments of the subject compound, or a pharmaceutically acceptable salt or solvate thereof, Q³ is independently C(R^{1d}). In some embodiments of the subject compound, or a pharmaceutically acceptable salt or solvate thereof, Q⁵ is independently N. In some embodiments of the subject compound, or a pharmaceutically acceptable salt or solvate thereof, Q⁵ is independently C(R^{1d}).

In some embodiments of the subject compound, or a pharmaceutically acceptable salt or solvate thereof, Q⁴ and Q⁶ are independently O, S, C(R^{1a})(R^{1b}), or N(R^{1c}). In some embodiments of the subject compound, or a pharmaceutically acceptable salt or solvate thereof, Q⁴ is independently O. In some embodiments of the subject compound, or a pharmaceutically acceptable salt or solvate thereof, Q⁴ is independently S. In some embodiments of the subject compound, or a pharmaceutically acceptable salt or solvate thereof, Q⁴ is independently C(R^{1a})(R^{1b}). In some embodiments of the subject compound, or a pharmaceutically acceptable salt or solvate thereof, Q⁴ is independently N(R^{1c}). In some embodiments of the subject compound, or a pharmaceutically acceptable salt or solvate thereof, Q⁶ is independently O. In some embodiments of the subject compound, or a pharmaceutically acceptable salt or solvate thereof, Q⁶ is independently S. In some embodiments of the subject compound, or a pharmaceutically acceptable salt or solvate thereof, Q⁶ is independently C(R^{1a})(R^{1b}). In some embodiments of the subject compound, or a pharmaceutically acceptable salt or solvate thereof, Q⁶ is independently N(R^{1c}).

In some embodiments of the subject compound, or a pharmaceutically acceptable salt or solvate thereof, X¹⁴, X¹⁵, X⁶, X⁹, X¹⁰, and X¹¹ are independently selected from C(R^{1a}) or N. In some embodiments of the subject compound, or a pharmaceutically acceptable salt or solvate thereof, X¹⁴ is independently C(R^{1a}). In some embodiments of the subject compound, or a pharmaceutically acceptable salt or solvate thereof, X¹⁴ is independently N. In some embodiments of the subject compound, or a pharmaceutically acceptable salt or solvate thereof, X¹⁵ is independently C(R^{1a}). In some embodiments of the subject compound, or a pharmaceutically acceptable salt or solvate thereof, X¹⁵ is independently N. In some embodiments of the subject compound, or a pharmaceutically acceptable salt or solvate thereof, X⁶ is independently C(R^{1a}). In some embodiments of the subject compound, or a pharmaceutically acceptable salt or solvate thereof, X⁶ is independently N. In some embodiments of the subject compound, or a pharmaceutically acceptable salt or solvate thereof, X⁹ is independently C(R^{1a}). In some embodiments of the subject compound, or a pharmaceutically acceptable salt or solvate thereof, X⁹ is independently N. In some embodiments of the subject compound, or a pharmaceutically acceptable salt or solvate thereof, X¹⁰ is independently C(R^{1a}). In some embodiments of the subject compound, or a pharmaceutically acceptable salt or solvate thereof, X¹⁰ is independently N. In some embodiments of the subject compound, or a pharmaceutically acceptable salt or solvate thereof, X¹¹ is independently C(R^{1a}). In some embodiments of the subject compound, or a pharmaceutically acceptable salt or solvate thereof, X¹¹ is independently N.

In some embodiments of the subject compound, or a pharmaceutically acceptable salt or solvate thereof, X⁷ and X⁸ are independently selected from C(R^{1a}), C(R^{1a})(R^{1b}), N, or N(R^{1c}). In some embodiments of the subject compound, or a pharmaceutically acceptable salt or solvate thereof, X⁷ is independently C(R^{1a}). In some embodiments of the subject compound, or a pharmaceutically acceptable salt or solvate thereof, X⁷ is independently C(R^{a})(R^{1b}). In some embodiments of the subject compound, or a pharmaceutically acceptable salt or solvate thereof, X⁷ is independently N. In some embodiments of the subject compound, or a pharmaceutically acceptable salt or solvate thereof, X⁷ is independently N(R^{1c}). In some embodiments of the subject compound, or a pharmaceutically acceptable salt or solvate thereof, X⁸ is independently C(R^{1a}). In some embodiments of the subject compound, or a pharmaceutically acceptable salt or solvate thereof, X⁸ is independently C(R^{1a})(R^{1b}). In some embodiments of the subject compound, or a pharmaceutically acceptable salt or solvate thereof, X⁸ is independently N. In some embodiments of the subject compound, or a pharmaceutically acceptable salt or solvate thereof, X⁸ is independently N(R^{1c}).

In some embodiments of the subject compound, or a pharmaceutically acceptable salt or solvate thereof, each R^{1a}, R^{1b}, R^{1d}, R^{1f}, R^{1g}, and R^{1h} are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ.

In some embodiments of the subject compound, or a pharmaceutically acceptable salt or solvate thereof, each R^{1a}, R^{1b}, R^{1d}, R^{1f}, R^{1g}, and R^{1h} are independently selected from hydrogen, halogen, C₁₋₆alkyl, C₁₋₆haloalkyl, —OR¹², —N(R¹²)(R¹³), wherein C₁₋₆alkyl is optionally substituted with one, two, or three R²⁰ⁱ. In some embodiments of the subject compound, or a pharmaceutically acceptable salt or solvate thereof, each R^{1a}, R^{1b}, R^{1d}, R^{1f}, R^{1g}, and R^{1h} are independently selected from hydrogen, halogen, C₁₋₆alkyl, C₁₋₆haloalkyl, —OH, —NH₂, wherein C₁₋₆alkyl is optionally substituted with one, two, or three R²⁰ⁱ. In some embodiments of the subject compound, or a pharmaceutically acceptable salt or solvate thereof, R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ. In some embodiments of the subject compound, or a pharmaceutically acceptable salt or solvate thereof, two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In some embodiments of the subject compound, or a pharmaceutically acceptable salt or solvate thereof, R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In some embodiments of the subject compound, or a pharmaceutically acceptable salt or solvate thereof, R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ.

In some embodiments of the subject compound, or a pharmaceutically acceptable salt or solvate thereof, each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₆heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ. In some embodiments of the subject compound, or a pharmaceutically acceptable salt or solvate thereof, each R^{1c} is independently hydrogen. In some embodiments of the subject compound, or a pharmaceutically acceptable salt or solvate thereof, each R^{1c} is independently C₁₋₆alkyl optionally substituted with one, two, or three R²⁰ⁱ.

In some embodiments of the subject compound, or a pharmaceutically acceptable salt or solvate thereof, each R²⁰ⁱ is independently halogen. In some embodiments of the subject compound, or a pharmaceutically acceptable salt or solvate thereof, each R²⁰ⁱ is independently —CN. In some embodiments of the subject compound, or a pharmaceutically acceptable salt or solvate thereof;
each R²⁰ⁱ is independently C₁₋₆alkyl. In some embodiments of the subject compound, or a pharmaceutically acceptable salt or solvate thereof, each R²⁰ⁱ is independently C₂₋₆alkenyl. In some embodiments of the subject compound, or a pharmaceutically acceptable salt or solvate thereof, each R²⁰ⁱ is independently C₂₋₆alkynyl. In some embodiments of the subject compound, or a pharmaceutically acceptable salt or solvate thereof, each R²⁰ⁱ is independently C₃₋₆cycloalkyl. In some embodiments of the subject compound, or a pharmaceutically acceptable salt or solvate thereof, each R²⁰ⁱ is independently —CH₂—C₃₋₆cycloalkyl. In some embodiments of the subject compound, or a pharmaceutically acceptable salt or solvate thereof, each R²⁰ⁱ is independently C₂₋₉heterocycloalkyl. In some embodiments of the subject compound, or a pharmaceutically acceptable salt or solvate thereof, each R²⁰ⁱ is independently —CH₂—C₂₋₉heterocycloalkyl. In some embodiments of the subject compound, or a pharmaceutically acceptable salt or solvate thereof, each R²⁰ⁱ is independently C₆₋₁₀aryl. In some embodiments of the subject compound, or a pharmaceutically acceptable salt or solvate thereof, each R²⁰ⁱ is independently —CH₂—C₆₋₁₀aryl. In some embodiments of the subject compound, or a pharmaceutically acceptable salt or solvate thereof, each R²⁰ⁱ is independently C₁₋₉heteroaryl. In some embodiments of the subject compound, or a pharmaceutically acceptable salt or solvate thereof, each R²⁰ⁱ is independently —OR²¹. In some embodiments of the subject compound, or a pharmaceutically acceptable salt or solvate thereof, each R²⁰ⁱ is independently —SR²¹. In some embodiments of the subject compound, or a pharmaceutically acceptable salt or solvate thereof, each R²⁰ⁱ is independently —N(R²²)(R²³). In some embodiments of the subject compound, or a pharmaceutically acceptable salt or solvate thereof, each R²⁰ⁱ is independently —C(O)OR²². In some embodiments of the subject compound, or a pharmaceutically acceptable salt or solvate thereof, each R²⁰ⁱ is independently —C(O)N(R²²)(R²³). In some embodiments of the subject compound, or a pharmaceutically acceptable salt or solvate thereof, each R²⁰ⁱ is independently —C(O)C(O)N(R²²)(R²³). In some embodiments of the subject compound, or a pharmaceutically acceptable salt or solvate thereof, each R²⁰ⁱ is independently —OC(O)N(R²²)(R²³). In some embodiments of the subject compound, or a pharmaceutically acceptable salt or solvate thereof, each R²⁰ⁱ is independently —N(R²⁴)C(O)N(R²²)(R²³). In some embodiments of the subject compound, or a pharmaceutically acceptable salt or solvate thereof, each R²⁰ⁱ is independently —N(R²⁴)C(O)OR²⁵. In some embodiments of the subject compound, or a pharmaceutically acceptable salt or solvate thereof, each R²⁰ⁱ is independently —N(R²⁴)C(O)R²⁵. In some embodiments of the subject compound, or a pharmaceutically acceptable salt or solvate thereof, each R²⁰ⁱ is independently —N(R²⁴)S(O)₂R²⁵. In some embodiments of the subject compound, or a pharmaceutically acceptable salt or solvate thereof, each R²⁰ⁱ is independently —C(O)R²⁵. In some embodiments of the subject compound, or a pharmaceutically acceptable salt or solvate thereof, each R²⁰ⁱ is independently —S(O)₂R²⁵. In some embodiments of the subject compound, or a pharmaceutically acceptable salt or solvate thereof, each R²⁰ⁱ is independently —S(O)₂N(R²²)(R²³). In some embodiments of the subject compound, or a pharmaceutically acceptable salt or solvate thereof, each R²⁰ⁱ is independently —OCH₂C(O)OR²². In some embodiments of the subject compound, or a pharmaceutically acceptable salt or solvate thereof, each R²⁰ⁱ is independently and —OC(O)R²⁵. In some embodiments of the subject compound, or a pharmaceutically acceptable salt or solvate thereof, each R²⁰ⁱ is independently C₁₋₆alkyl. In some embodiments of the subject compound, or a pharmaceutically acceptable salt or solvate thereof, each R²⁰ⁱ is independently C₂₋₆alkenyl. In some embodiments of the subject compound, or a pharmaceutically acceptable salt or solvate thereof, each R²⁰ⁱ is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently C₃₋₆cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently —CH₂—C₃₋₆cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently —CH₂—C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In some embodiments of the subject compound, or a pharmaceutically acceptable salt or solvate thereof, each R²⁰ⁱ is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments, R² is hydrogen. In embodiments, R² is halogen. In embodiments, R² is —CN. In embodiments, R² is C₁₋₆alkyl. In embodiments, R² is C₂₋₆alkenyl. In embodiments, R² is C₂₋₆alkynyl. In embodiments, R² is C₃₋₁₀cycloalkyl. In embodiments, R² is C₂₋₉heterocycloalkyl. In embodiments, R² is C₆₋₁₀aryl. In embodiments, R² is C₁₋₉heteroaryl.

In embodiments, R² is C₁₋₆alkyl optionally substituted with one, two, or three R^{20d}. In embodiments, R² is C₂₋₆alkenyl optionally substituted with one, two, or three R^{20d}. In embodiments, R² is C₂₋₆alkynyl optionally substituted with one, two, or three R^{20d}. In embodiments, R² is C₃₋₁₀cycloalkyl optionally substituted with one, two, or three R^{20d}. In embodiments, R² is C₂₋₉heterocycloalkyl optionally substituted with one, two, or three R^{20d}. In embodiments, R² is C₆₋₁₀aryl optionally substituted with one, two, or three R^{20d}. In embodiments, R² is C₁₋₉heteroaryl optionally substituted with one, two, or three R^{20d}.

In embodiments, R² is —OR¹². In embodiments, R² is —SR¹². In embodiments, R² is —N(R¹²)(R¹³). In embodiments, R² is —C(O)OR¹². In embodiments, R² is —OC(O)N(R¹²)(R¹³). In embodiments, R² is —N(R¹⁴)C(O)N(R¹²)(R¹³). In embodiments, R² is —N(R¹⁴)C(O)OR¹⁵. In embodiments, R² is —N(R¹⁴)S(O)₂R¹⁵. In embodiments, R² is —C(O)R¹⁵. In embodiments, R² is —S(O)R¹⁵. In embodiments, R² is —OC(O)R¹⁵. In embodiments, R² is —C(O)N(R¹²)(R¹³). In embodiments, R² is —C(O)C(O)N(R¹²)(R¹³). In embodiments, R² is —N(R¹⁴)C(O)R¹⁵. In embodiments, R² is —S(O)₂R¹⁵. In embodiments, R² is —S(O)₂N(R¹²)(R¹³). In embodiments, R² is S(═OX(═NH)N(R¹²)(R¹³). In embodiments, R² is —CH₂C(O)N(R¹²)(R¹³). In embodiments, R² is —CH₂N(R¹⁴)C(O)R¹⁵. In embodiments, R² is —CH₂S(O)₂R¹⁵. In embodiments, R² is —CH₂S(O)₂N(R¹²)(R¹³).

In some embodiments the compound of Formula (I′), or a pharmaceutically acceptable salt or solvate thereof, has the formula:

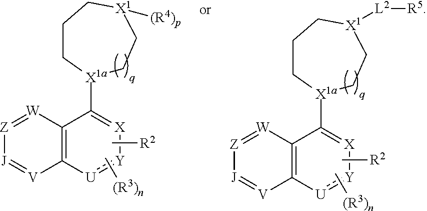

In some embodiments the compound of Formula (Ia), or a pharmaceutically acceptable salt or solvate thereof, has the formula:

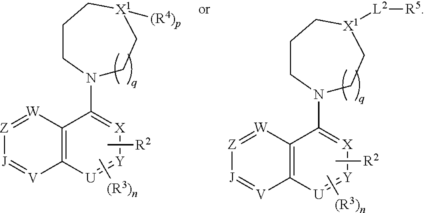

In some embodiments the compound of Formula (Ib), or a pharmaceutically acceptable salt or solvate thereof, has the formula:

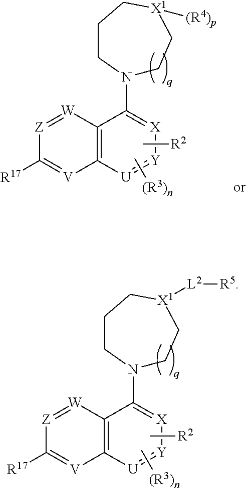

In some embodiments the compound of Formula (Ic), or a pharmaceutically acceptable salt or solvate thereof, has the formula:

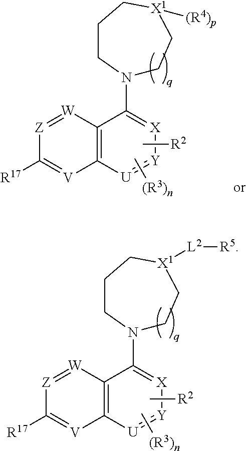

In some embodiments the compound of Formula (Id), or a pharmaceutically acceptable salt or solvate thereof, has the formula:

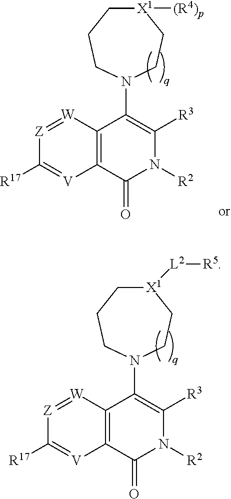

In some embodiments the compound of Formula (Ie), or a pharmaceutically acceptable salt or solvate thereof, has the formula:

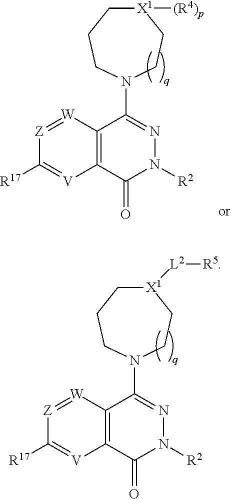

In some embodiments the compound of Formula (If), or a pharmaceutically acceptable salt or solvate thereof, has the formula:

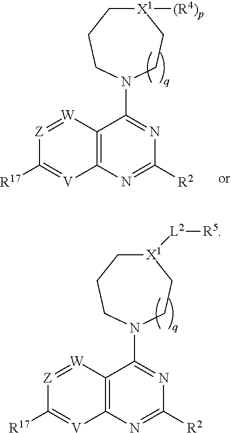

In some embodiments the compound of Formula (Ig), or a pharmaceutically acceptable salt or solvate thereof, has the formula:

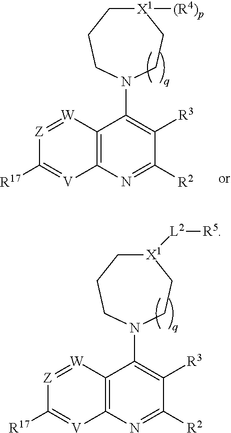

In some embodiments the compound of Formula (I′), or a pharmaceutically acceptable salt or solvate thereof, has the formula:

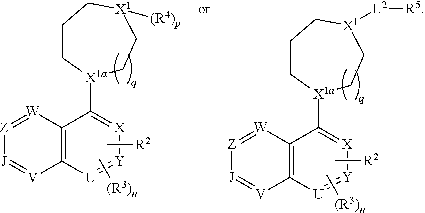

In some embodiments the compound of Formula (I″-1) or (T″-2), or a pharmaceutically acceptable salt or solvate thereof, has the formula:

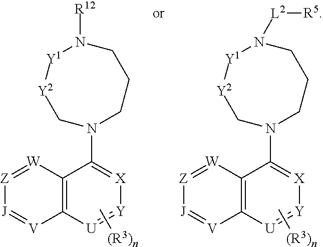

In some embodiments the compound of Formula (IIc′), or a pharmaceutically acceptable salt or solvate thereof, has the formula:

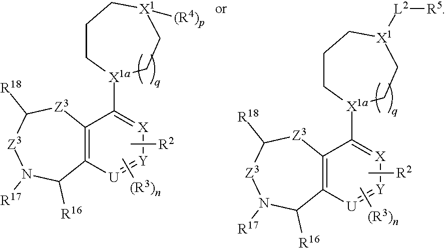

In some embodiments the compound of Formula (IIc), or a pharmaceutically acceptable salt or solvate thereof, has the

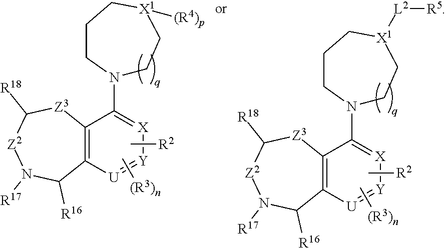

In some embodiments the compound of Formula (IId′), or a pharmaceutically acceptable salt or solvate thereof, has the formula;

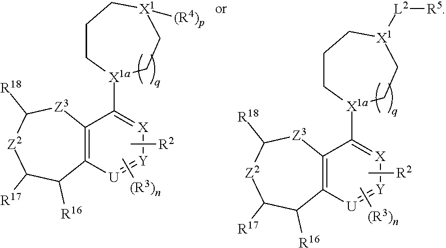

In some embodiments the compound of Formula (IId), or a pharmaceutically acceptable salt or solvate thereof, has the formula:

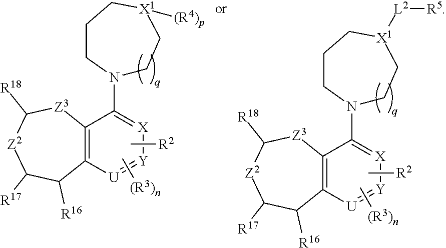

In some embodiments the compound of Formula (Le), or a pharmaceutically acceptable salt or solvate thereof, has the formula:

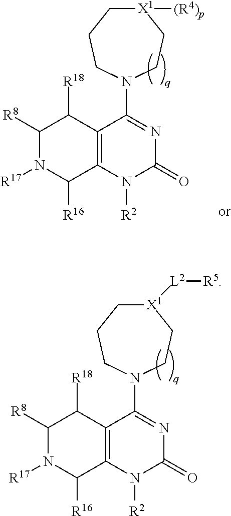

In some embodiments the compound of Formula (IIf), or a pharmaceutically acceptable salt or solvate thereof, has the formula:

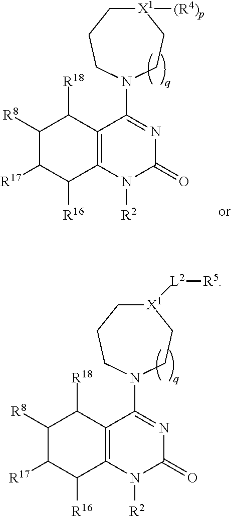

In some embodiments the compound of Formula (IIg), or a pharmaceutically acceptable salt or solvate thereof, has the formula:

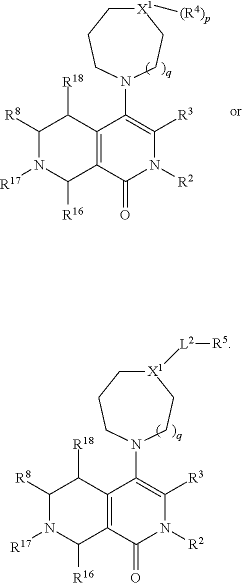

In some embodiments the compound of Formula (IIh), or a pharmaceutically acceptable salt or solvate thereof, has the formula:

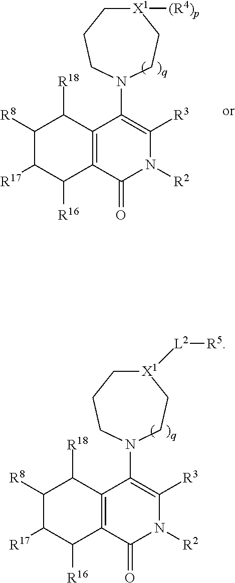

In some embodiments the compound of Formula (III), or a pharmaceutically acceptable salt or solvate thereof, has the formula:

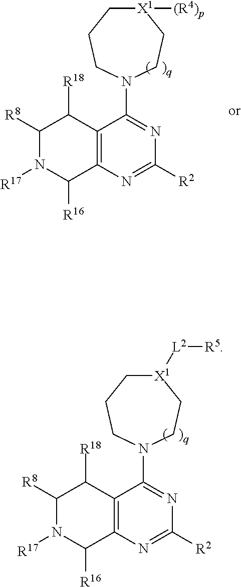

In some embodiments the compound of Formula (IIj), or a pharmaceutically acceptable salt or solvate thereof, has the formula:

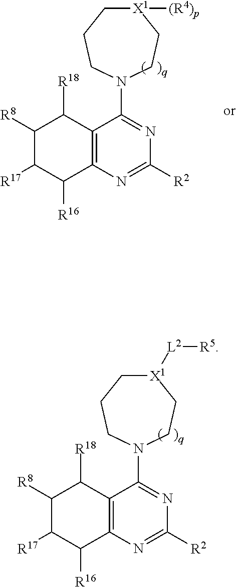

In some embodiments the compound of Formula (II), or a pharmaceutically acceptable salt or solvate thereof, has the formula:

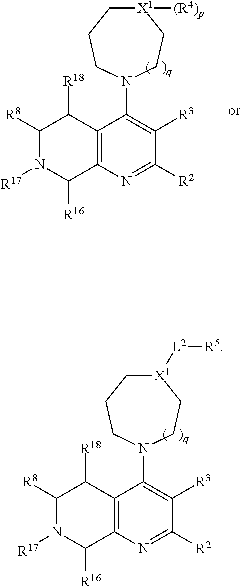

In some embodiments the compound of Formula (IIm), or a pharmaceutically acceptable salt or solvate thereof, has the formula:

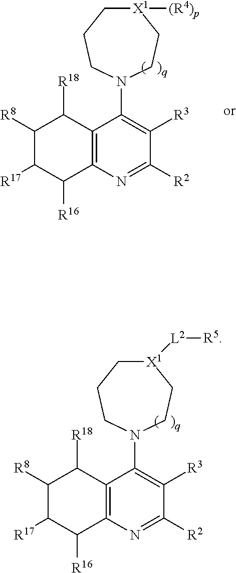

In some embodiments the compound of Formula (IIIa-1), (IIIa-2), (IIIa-3), or (IIIa-4), or a pharmaceutically acceptable salt or solvate thereof, has the formula:

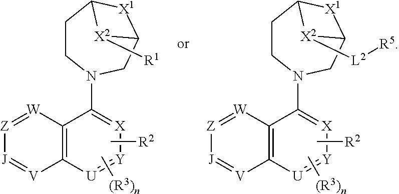

In some embodiments the compound of Formula (IIIb-1), (IIIb-2), (IIIb-3), or (IIIb-4), or a pharmaceutically acceptable salt or solvate thereof, has the formula:

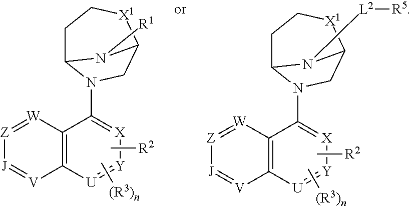

In some embodiments the compound of Formula (IIId-1), (IIId-2), (IIId-3), or (IIId-4), or a pharmaceutically acceptable salt or solvate thereof, has the formula:

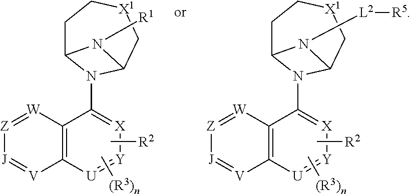

In some embodiments the compound of Formula (IIIc-1), (IIIc-2), (IIIc-3), or (IIIc-4), or a pharmaceutically acceptable salt or solvate thereof, has the formula:

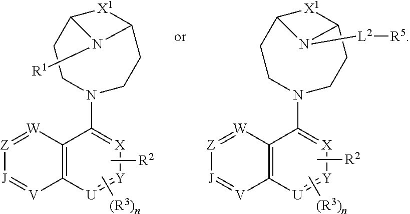

In some embodiments the compound of Formula (IIIf-1), (IIIf-2), (IIIf-3), or (IIIf-4), or a pharmaceutically acceptable salt or solvate thereof, has the formula:

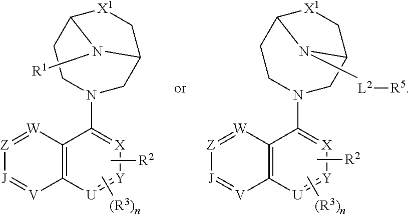

In some embodiments the compound of Formula (IIIg-1), (IIIg-2), (IIIg-3), or (IIIg-4), or a pharmaceutically acceptable salt or solvate thereof, has the formula:

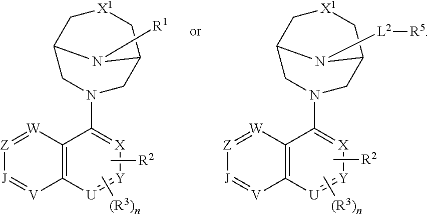

In some embodiments the compound of Formula (IIIh-1), (IIIh-2), (IIIh-3), or (IIIh-4), or a pharmaceutically acceptable salt or solvate thereof, has the formula:

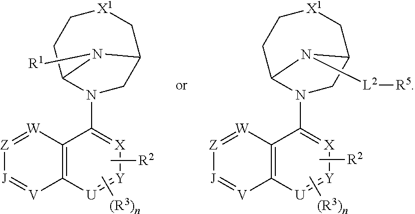

In some embodiments the compound of Formula (IIIi-1), (IIIi-2), (IIIi-3), or (IIIi-4), or a pharmaceutically acceptable salt or solvate thereof, has the formula:

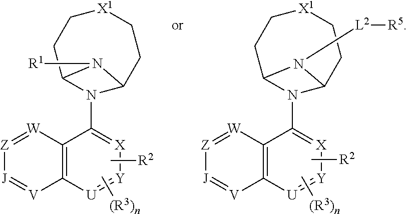

In some embodiments the compound of Formula (IIIc-1), (IIIc-2), (IIIc-3), or (IIIc-4), or a pharmaceutically acceptable salt or solvate thereof, has the formula:

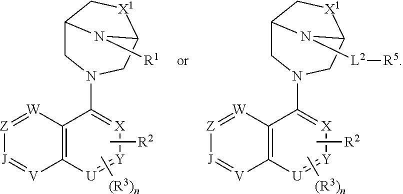

In some embodiments the compound of Formula (IVa-1), (IVb-1), (IVc-1), (IVa-2), (IVb-2), or (IVc-2), or a pharmaceutically acceptable salt or solvate thereof, has the formula

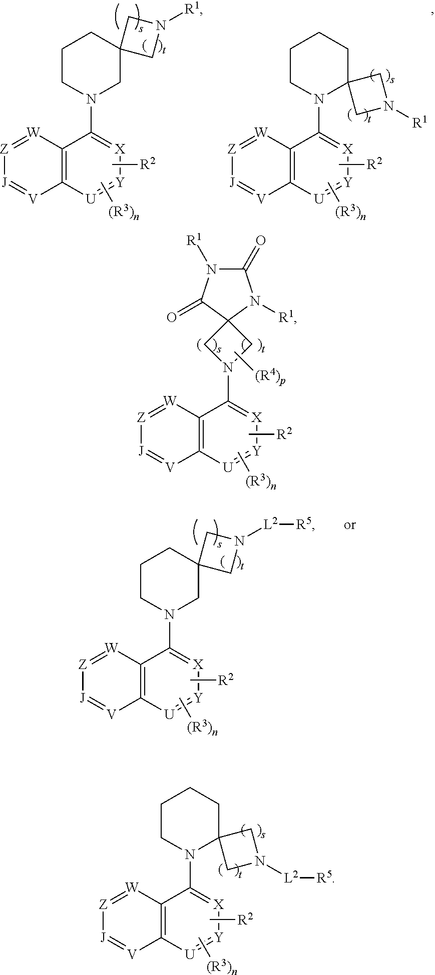

In some embodiments the compound of Formula (Va-1), (Vb-1), (Vc-1), (Va-2), (Vb-2), or (Vc-2), or a pharmaceutically acceptable salt or solvate thereof, has the formula

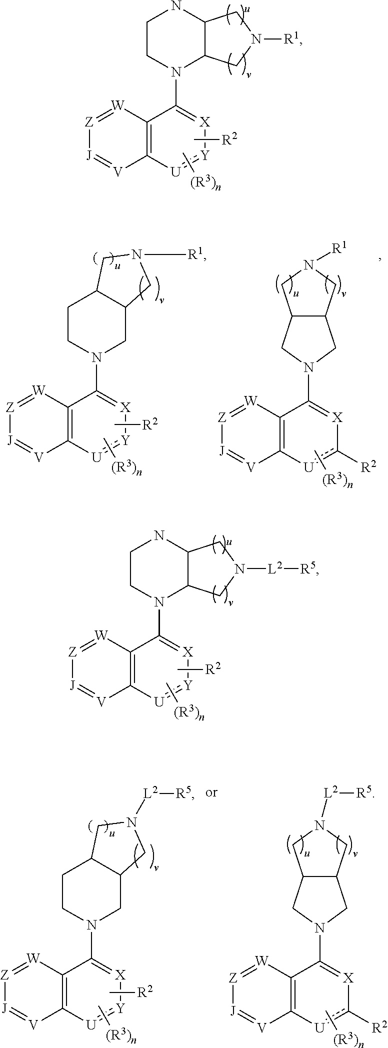

In some embodiments the compound of Formula (XI-1), or a pharmaceutically acceptable salt or solvate thereof, has the formula

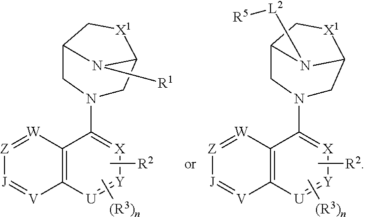

In some embodiments is a compound of Formula (XIa), or a pharmaceutically acceptable salt or solvate thereof, has the formula

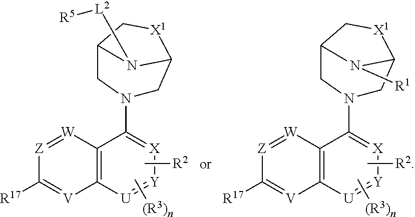

In some embodiments the compound of Formula (XIb), or a pharmaceutically acceptable salt or solvate thereof, has the formula

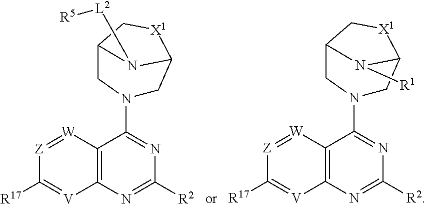

In some embodiments the compound of Formula (XIc), or a pharmaceutically acceptable salt or solvate thereof, has the formula

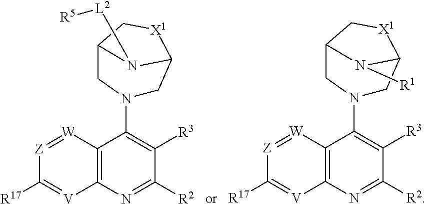

In some embodiments the compound of Formula (XId), or a pharmaceutically acceptable salt or solvate thereof, has the formula

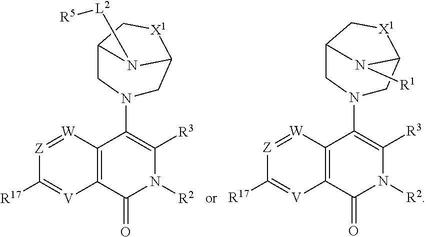

In some embodiments the compound of Formula (XIe), or a pharmaceutically acceptable salt or solvate thereof, has the formula

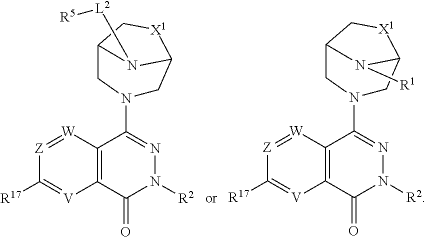

In some embodiments the compound of Formula (XIf), or a pharmaceutically acceptable salt or solvate thereof, has the formula

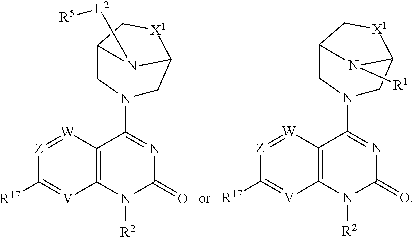

In some embodiments the compound of Formula (XXI-1) or (XXI-2), or a pharmaceutically acceptable salt or solvate thereof, has the formula:

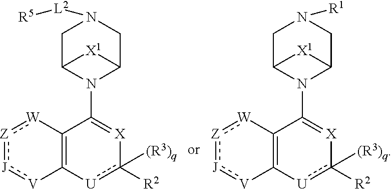

In some embodiments the compound of Formula (XXIa), or a pharmaceutically acceptable salt or solvate thereof, has the formula:

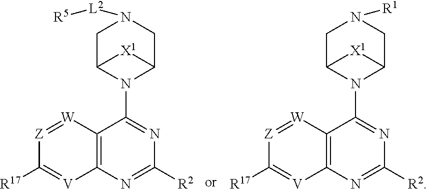

In some embodiments the compound of Formula (XXIb), or a pharmaceutically acceptable salt or solvate thereof, has the

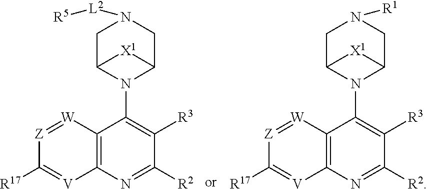

In some embodiments the compound of Formula (XXIc), or a pharmaceutically acceptable salt or solvate thereof, has the formula:

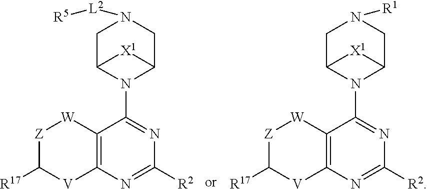

In some embodiments the compound of Formula (XXII-1) or (XXII-2), or a pharmaceutically acceptable salt or solvate thereof, has the formula:

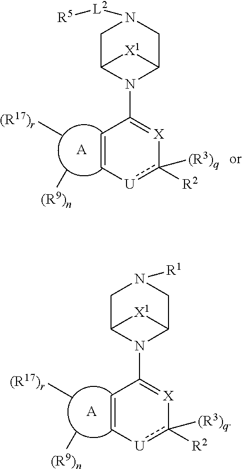

In some embodiments the compound of Formula (XXIIa-1) or (XXIIa-2), or a pharmaceutically acceptable salt or solvate thereof, has the formula:

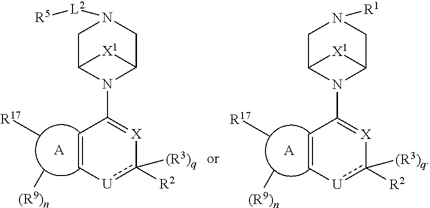

In some embodiments the compound of Formula (XXIIb-1) or (XXIIb-2), or a pharmaceutically acceptable salt or solvate thereof, has the formula:

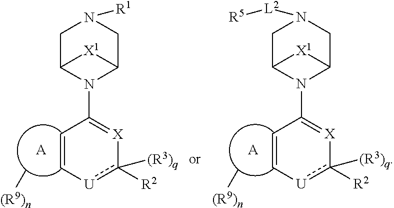

In some embodiments the compound of Formula (XXIIc), or a pharmaceutically acceptable salt or solvate thereof, has the formula:

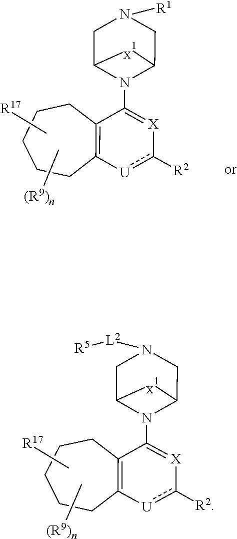

In some embodiments the compound of Formula (XXIId), or a pharmaceutically acceptable salt or solvate thereof, has the

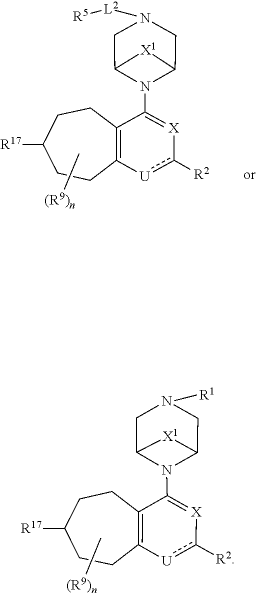

In some embodiments the compound of Formula (XXIIe), or a pharmaceutically acceptable salt or solvate thereof, has the formula:

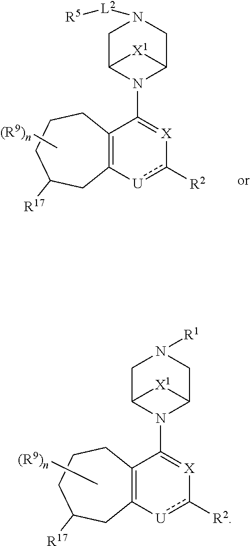

In some embodiments the compound of Formula (XXIIf), or a pharmaceutically acceptable salt or solvate thereof, has the formula:

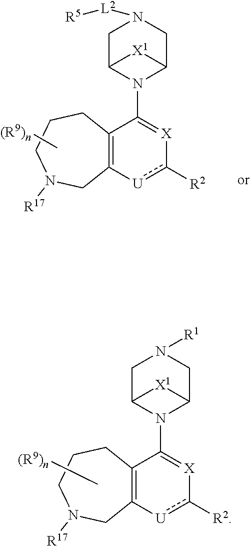

In some embodiments the compound of Formula (XXIIg), or a pharmaceutically acceptable salt or solvate thereof, has the

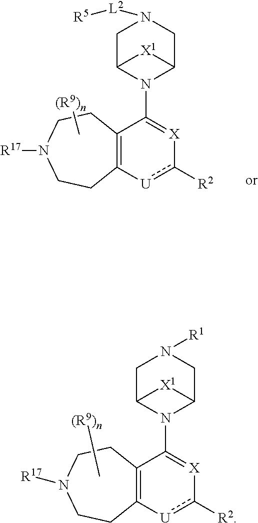

In some embodiments the compound of Formula (XXIII-1) or (XXIII-2), or a pharmaceutically acceptable salt or solvate thereof, has the formula:

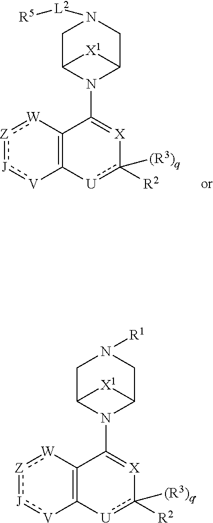

In some embodiments the compound of Formula (XXIV-1) or (XXIV-2), or a pharmaceutically acceptable salt or solvate thereof, has the formula

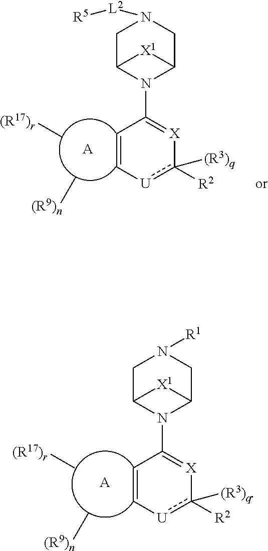

In some embodiments, the disclosure provides a compound selected from:

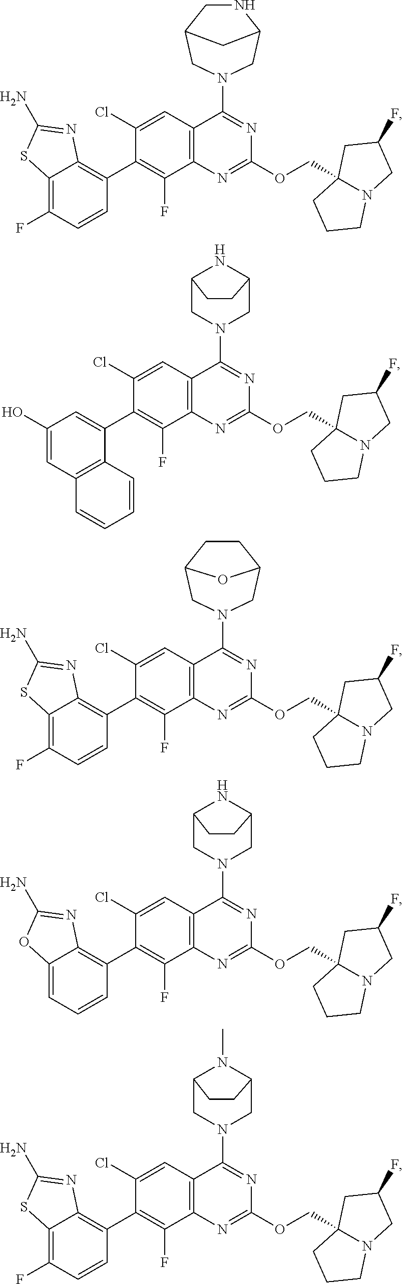 [SMILES: Nc1nc2c(-c3c(Cl)cc4c(N5CC6CNC(C6)C5)nc(OC[C@@]56CCCN5C[C@H](F)C6)nc4c3F)ccc(F)c2s1 ; Oc1cc(-c2c(Cl)cc3c(N4CC5CCC(C4)N5)nc(OC[C@@]45CCCN4C[C@H](F)C5)nc3c2F)c2ccccc2c1 ; Nc1nc2c(-c3c(Cl)cc4c(N5CC6CCC(C5)O6)nc(OC[C@@]56CCCN5C[C@H](F)C6)nc4c3F)ccc(F)c2s1 ; Nc1nc2c(-c3c(Cl)cc4c(N5CC6CCC(C5)N6)nc(OC[C@@]56CCCN5C[C@H](F)C6)nc4c3F)cccc2o1 ; CN1C2CCC1CN(c1nc(OC[C@@]34CCCN3C[C@H](F)C4)nc3c(F)c(-c4ccc(F)c5sc(N)nc45)c(Cl)cc13)C2]

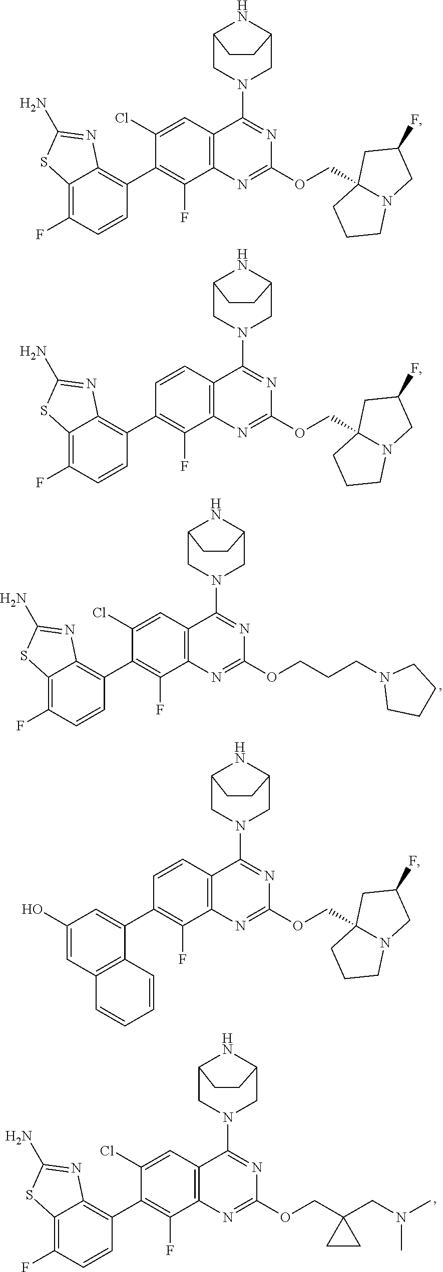 [SMILES: Nc1nc2c(-c3c(Cl)cc4c(N5CC6CCC(C5)N6)nc(OC[C@@]56CCCN5C[C@H](F)C6)nc4c3F)ccc(F)c2s1 ; Nc1nc2c(-c3ccc4c(N5CC6CCC(C5)N6)nc(OC[C@@]56CCCN5C[C@H](F)C6)nc4c3F)ccc(F)c2s1 ; Nc1nc2c(-c3c(Cl)cc4c(N5CC6CCC(C5)N6)nc(OCCCN5CCCC5)nc4c3F)ccc(F)c2s1 ; Oc1cc(-c2ccc3c(N4CC5CCC(C4)N5)nc(OC[C@@]45CCCN4C[C@H](F)C5)nc3c2F)c2ccccc2c1 ; CN(C)CC1(COc2nc(N3CC4CCC(C3)N4)c3cc(Cl)c(-c4ccc(F)c5sc(N)nc45)c(F)c3n2)CC1]

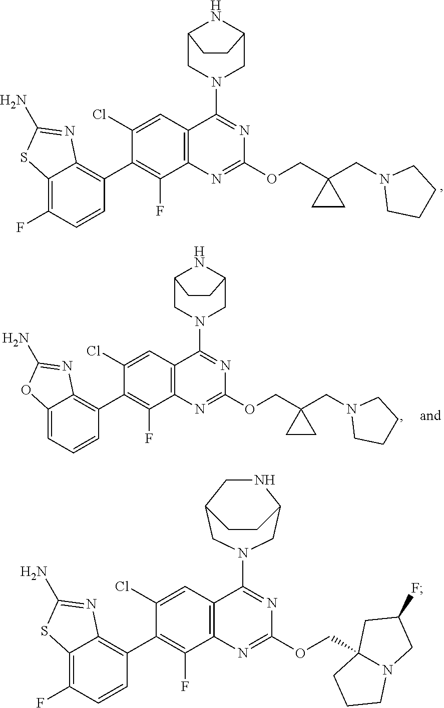 [SMILES: Nc1nc2c(-c3c(Cl)cc4c(N5CC6CCC(C5)N6)nc(OCC5(CN6CCCC6)CC5)nc4c3F)ccc(F)c2s1 ; Nc1nc2c(-c3c(Cl)cc4c(N5CC6CCC(C5)N6)nc(OCC5(CN6CCCC6)CC5)nc4c3F)cccc2o1 ; Nc1nc2c(-c3c(Cl)cc4c(N5CC6CCC(C5)NC6)nc(OC[C@@]56CCCN5C[C@H](F)C6)nc4c3F)ccc(F)c2s1]

or a pharmaceutically acceptable salt or solvate thereof.

In some embodiments, the compounds of Formula (XI) disclosed herein include a compound having the structure:

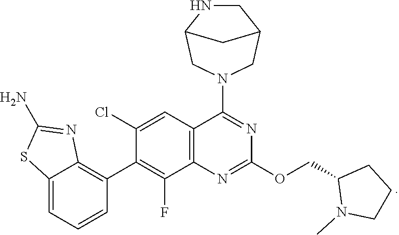 [SMILES: CN1CCC[C@H]1COc1nc(N2CC3CNC(C3)C2)c2cc(Cl)c(-c3cccc4sc(N)nc34)c(F)c2n1]

In some embodiments, the compounds of Formula (XI) disclosed herein do not include a compound having the structure:

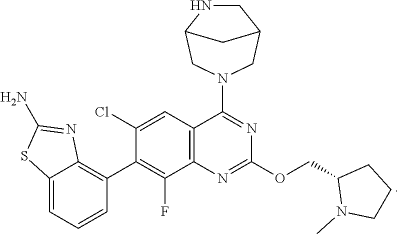 [SMILES: CN1CCC[C@H]1COc1nc(N2CC3CNC(C3)C2)c2cc(Cl)c(-c3cccc4sc(N)nc34)c(F)c2n1]

| Compound | Structure | Name |
|---|---|---|
| 1 | 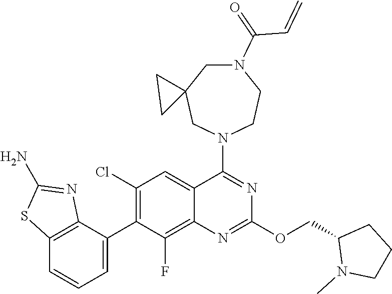 [SMILES: C=CC(=O)N1CCN(c2nc(OC[C@@H]3CCCN3C)nc3c(F)c(-c4cccc5sc(N)nc45)c(Cl)cc23)CC2(CC2)C1] | 1-(8-(7-(2-aminobenzo[d]thiazol- 4-yl)-6-chloro-8-fluoro-2-(((S)-1- methylpyrrolidin-2-yl)methoxy)- quinazolin-4-yl)-5,8-diazaspiro- [2.6]nonan-5-yl)prop-2-en-1-one |
| 2 | 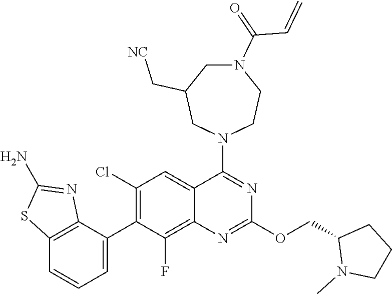 [SMILES: C=CC(=O)N1CCN(c2nc(OC[C@@H]3CCCN3C)nc3c(F)c(-c4cccc5sc(N)nc45)c(Cl)cc23)CC(CC#N)C1] | 2-(1-acryloyl-4-(7-(2-amino- benzo[d]thiazol-4-yl)-6-chloro-8- fluoro-2-(((S)-1-methylpyrrolidin- 2-yl)methoxy)quinazolin-4-yl)- 1,4-diazepan-6-yl)acetonitrile |
| 3 | 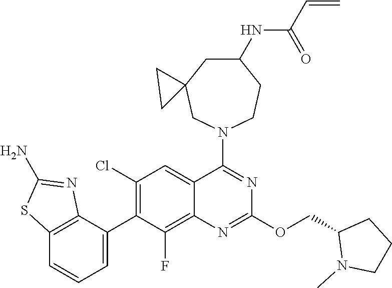 [SMILES: C=CC(=O)NC1CCN(c2nc(OC[C@@H]3CCCN3C)nc3c(F)c(-c4cccc5sc(N)nc45)c(Cl)cc23)CC2(CC2)C1] | N-(1-(7-(2-aminobenzo[d]thiazol- 4-yl)-6-chloro-8-fluoro-2-(((S)-1- methylpyrrolidin-2-yl)methoxy)- quinazolin-4-yl)azepan-4-yl)- acrylamide |
| 4 | 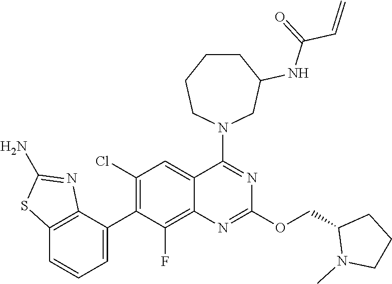 [SMILES: C=CC(=O)NC1CCCCN(c2nc(OC[C@@H]3CCCN3C)nc3c(F)c(-c4cccc5sc(N)nc45)c(Cl)cc23)C1] | N-(1-(7-(2-aminobenzo[d]thiazol- 4-yl)-6-chloro-8-fluoro-2-(((S)-1- methylpyrrolidin-2-yl)methoxy)- quinazolin-4-yl)azepan-3-yl)- acrylamide |
| 5 | 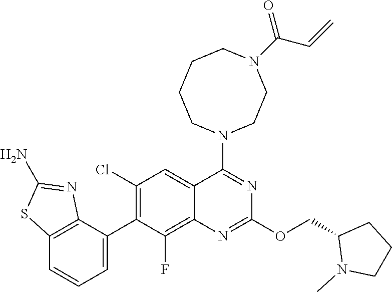 [SMILES: C=CC(=O)N1CCCCN(c2nc(OC[C@@H]3CCCN3C)nc3c(F)c(-c4cccc5sc(N)nc45)c(Cl)cc23)CC1] | 1-(4-(7-(2-aminobenzo[d]thiazol- 4-yl)-6-chloro-8-fluoro-2-(((S)-1- methylpyrrolidin-2-yl)methoxy)- quinazolin-4-yl)-1,4-diazocan-1- yl)prop-2-en-1-one |
| 6 | 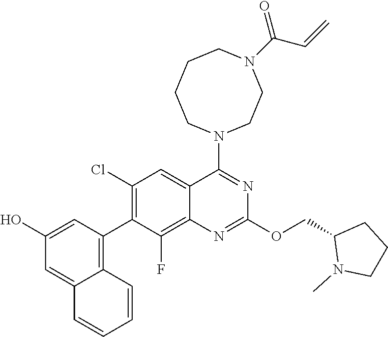 [SMILES: C=CC(=O)N1CCCCN(c2nc(OC[C@@H]3CCCN3C)nc3c(F)c(-c4cc(O)cc5ccccc45)c(Cl)cc23)CC1] | 1-(4-(6-chloro-8-fluoro-7-(3- hydroxynaphthalen-1-yl)-2- (((S)-1-methylpyrrolidin-2- yl)methoxy)quinazolin-4-yl)- 1,4-diazocan-1-yl)prop-2-en- 1-one |
| 7 | 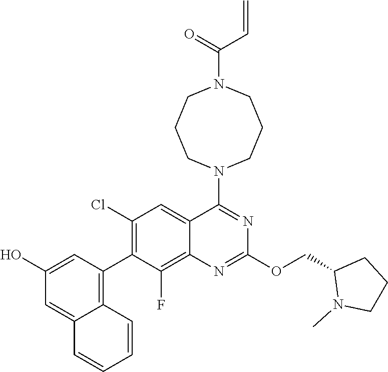 [SMILES: C=CC(=O)N1CCCN(c2nc(OC[C@@H]3CCCN3C)nc3c(F)c(-c4cc(O)cc5ccccc45)c(Cl)cc23)CCC1] | 1-(5-(6-chloro-8-fluoro-7-(3- hydroxynaphthalen-1-yl)-2-(((S)- 1-methylpyrrolidin-2-yl)meth- oxy)quinazolin-4-yl)-1,5-diazo- can-1-yl)prop-2-en-1-one |
| 8 | 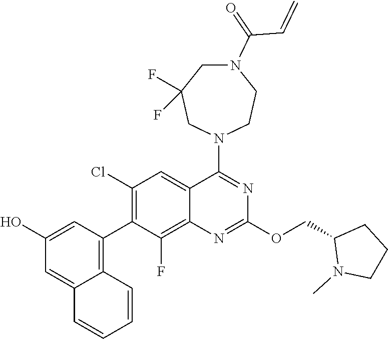 [SMILES: C=CC(=O)N1CCN(c2nc(OC[C@@H]3CCCN3C)nc3c(F)c(-c4cc(O)cc5ccccc45)c(Cl)cc23)CC(F)(F)C1] | 1-(4-(6-chloro-8-fluoro-7-(3- hydroxynaphthalen-1-yl)-2-(((S)- 1-methylpyrrolidin-2-yl)meth- oxy)quinazolin-4-yl)-6,6-difluoro- 1,4-diazepan-1-yl)prop-2-en-1- one |
| 9 | 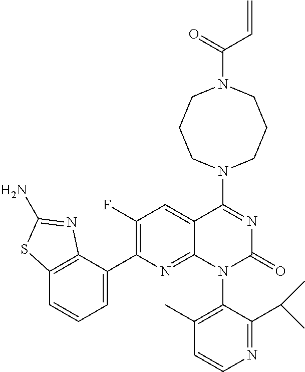 [SMILES: C=CC(=O)N1CCCN(c2nc(=O)n(-c3c(C)ccnc3C(C)C)c3nc(-c4cccc5sc(N)nc45)c(F)cc23)CCC1] | 4-(5-acryloyl-1,5-diazocan-1-yl)- 7-(2-aminobenzo[d]thiazol-4-yl)- 6-fluoro-1-(2-isopropyl-4-methyl- pyridin-3-yl)pyrido[2,3-d]pyrim- idin-2(1H)-one |
| 10 | 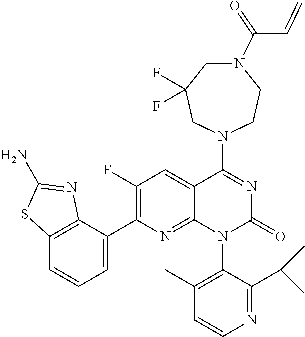 [SMILES: C=CC(=O)N1CCN(c2nc(=O)n(-c3c(C)ccnc3C(C)C)c3nc(-c4cccc5sc(N)nc45)c(F)cc23)CC(F)(F)C1] | 4-(4-acryloyl-6,6-difluoro-1,4- diazepan-1-yl)-7-(2-aminobenzo- [d]thiazol-4-yl)-6-fluoro-1-(2- isopropyl-4-methylpyridin-3-yl)- pyrido[2,3-d]pyrimidin-2(1H)- one |
| 11 | 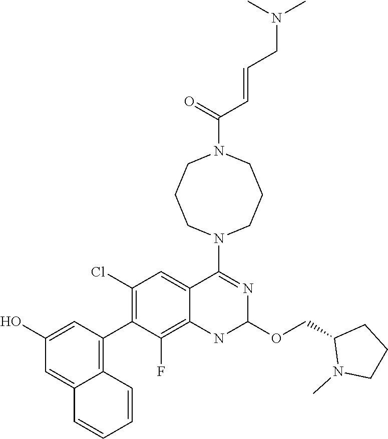 [SMILES: CN(C)C/C=C/C(=O)N1CCCN(C2=NC(OC[C@@H]3CCCN3C)Nc3c2cc(Cl)c(-c2cc(O)cc4ccccc24)c3F)CCC1] | (E)-1-(5-(6-chloro-8-fluoro-7-(3- hydroxynaphthalen-1-yl)-2-(((S)- 1-methylpyrrolidin-2-yl)meth- oxy)quinazolin-4-yl)-1,5-diazo- can-1-yl)-4-(dimethylamino)but- 2-en-1-one |
| 12 | 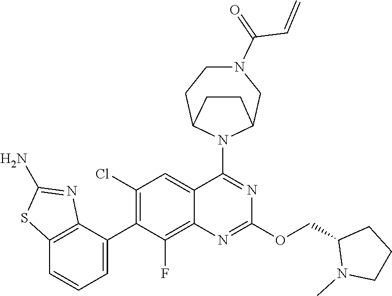 [SMILES: C=CC(=O)N1CCC2CCC(C1)N2c1nc(OC[C@@H]2CCCN2C)nc2c(F)c(-c3cccc4sc(N)nc34)c(Cl)cc12] | 1-(9-(7-(2-aminobenzo[d]thiazol- 4-yl)-6-chloro-8-fluoro-2-(((S)-1- methylpyrrolidin-2-yl)methoxy)- quinazolin-4-yl)-3,9-diazabicyclo- [4.2.1]nonan-3-yl)-prop-2-en-1- one |
| 13 | 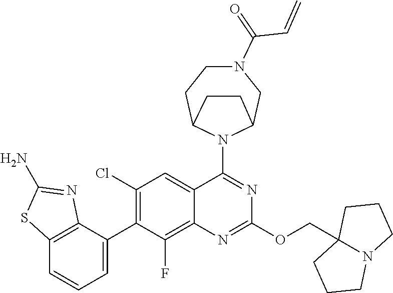 [SMILES: C=CC(=O)N1CCC2CCC(C1)N2c1nc(OCC23CCCN2CCC3)nc2c(F)c(-c3cccc4sc(N)nc34)c(Cl)cc12] | 1-(9-(7-(2-aminobenzo[d]thiazol- 4-yl)-6-chloro-8-fluoro-2-((tetra- hydro-1H-pyrrolizin-7a(5H)-yl)- methoxy)quinazolin-4-yl)-3,9- diazabicyclo[4.2.1]nonan-3-yl)- prop-2-en-1-one |
| 14 | 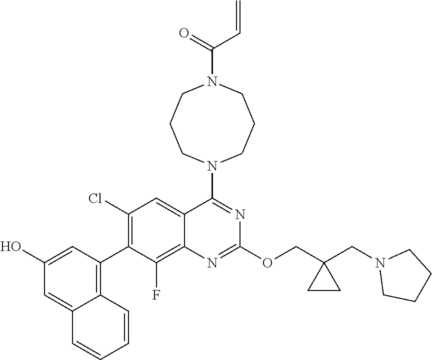 [SMILES: C=CC(=O)N1CCCN(c2nc(OCC3(CN4CCCC4)CC3)nc3c(F)c(-c4cc(O)cc5ccccc45)c(Cl)cc23)CCC1] | 1-(5-(6-chloro-8-fluoro-7-(3- hydroxynaphthalen-1-yl)-2-((1- (pyrrolidin-1-ylmethyl)cyclo- propyl)methoxy)quinazolin-4- yl)-1,5-diazocan-1-yl)prop-2-en- 1-one |
| 15 | 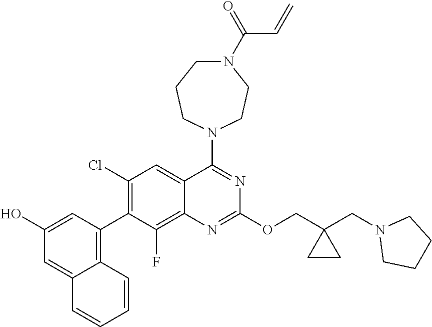 [SMILES: C=CC(=O)N1CCCN(c2nc(OCC3(CN4CCCC4)CC3)nc3c(F)c(-c4cc(O)cc5ccccc45)c(Cl)cc23)CC1] | 1-(4-(6-chloro-8-fluoro-7-(3- hydroxynaphthalen-1-yl)-2-((1- (pyrrolidin-1-ylmethyl)cyclo- propyl)methoxy)quinazolin-4- yl)-1,4-diazepan-1-yl)prop-2-en- 1-one |

In embodiments, the compound is selected from

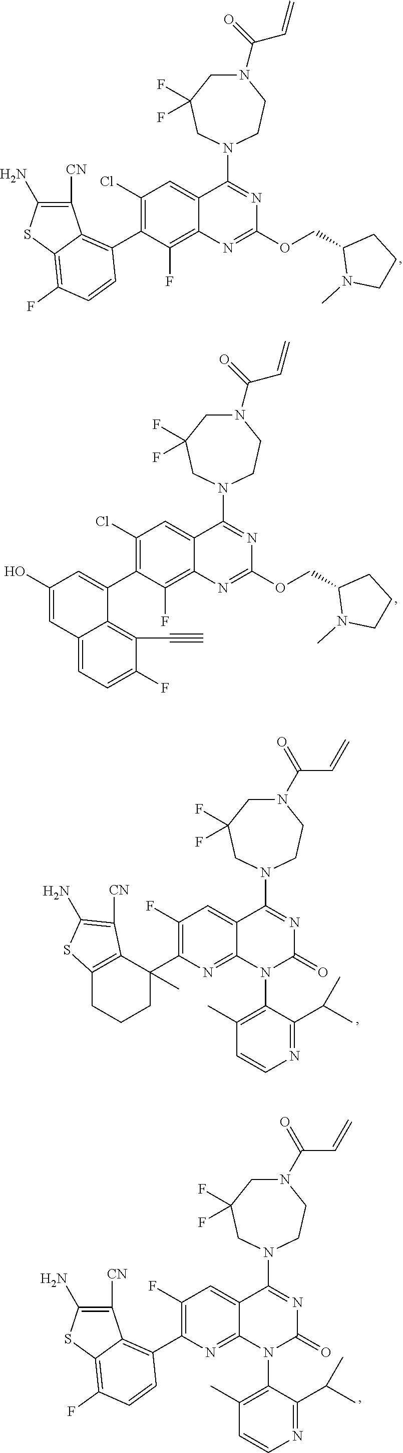 [SMILES: C=CC(=O)N1CCN(c2nc(OC[C@@H]3CCCN3C)nc3c(F)c(-c4ccc(F)c5sc(N)c(C#N)c45)c(Cl)cc23)CC(F)(F)C1 ; C#Cc1c(F)ccc2cc(O)cc(-c3c(Cl)cc4c(N5CCN(C(=O)C=C)CC(F)(F)C5)nc(OC[C@@H]5CCCN5C)nc4c3F)c12 ; C=CC(=O)N1CCN(c2nc(=O)n(-c3c(C)ccnc3C(C)C)c3nc(C4(C)CCCc5sc(N)c(C#N)c54)c(F)cc23)CC(F)(F)C1 ; C=CC(=O)N1CCN(c2nc(=O)n(-c3c(C)ccnc3C(C)C)c3nc(-c4ccc(F)c5sc(N)c(C#N)c45)c(F)cc23)CC(F)(F)C1]

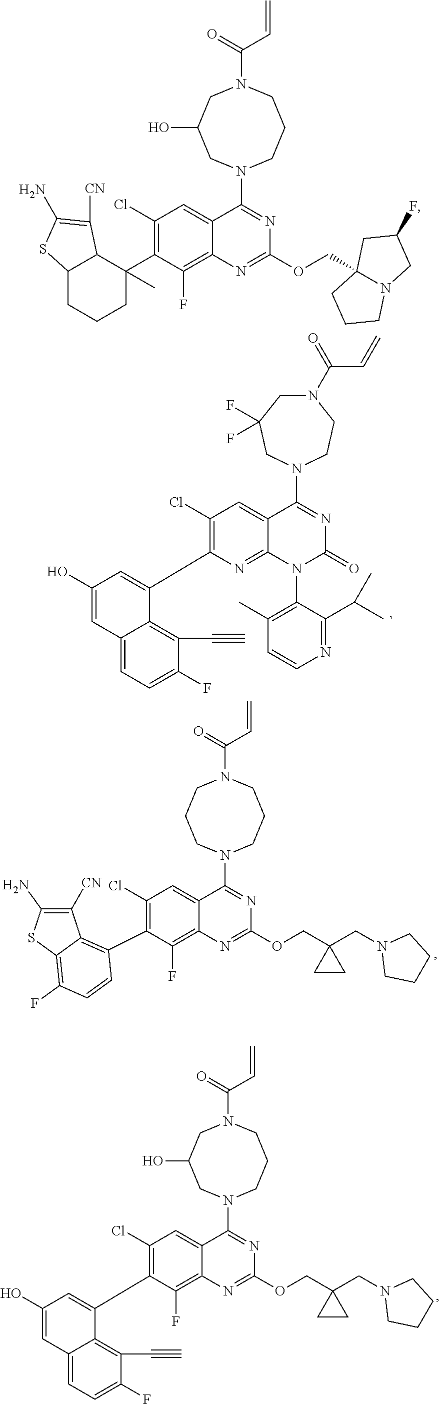

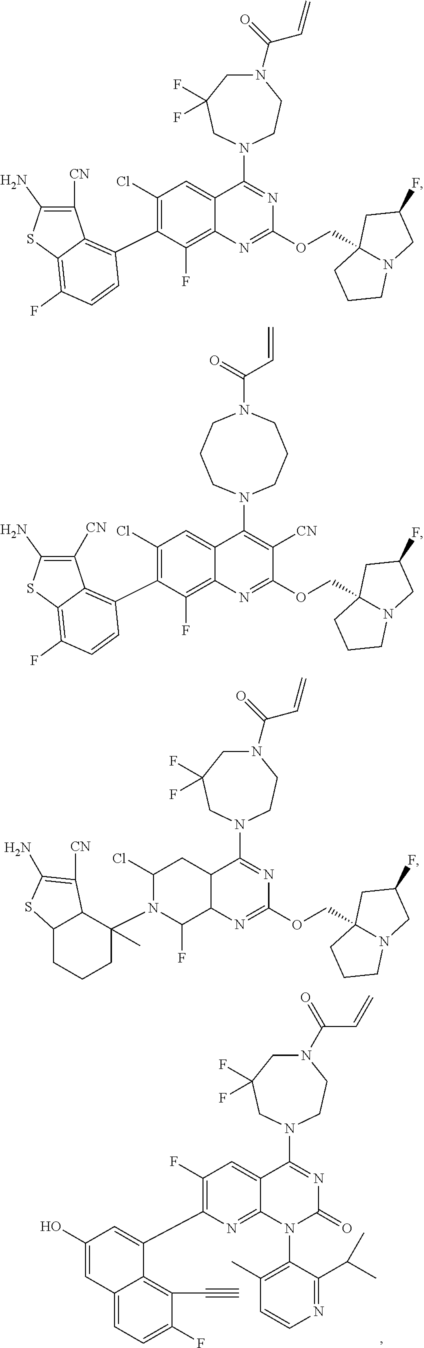

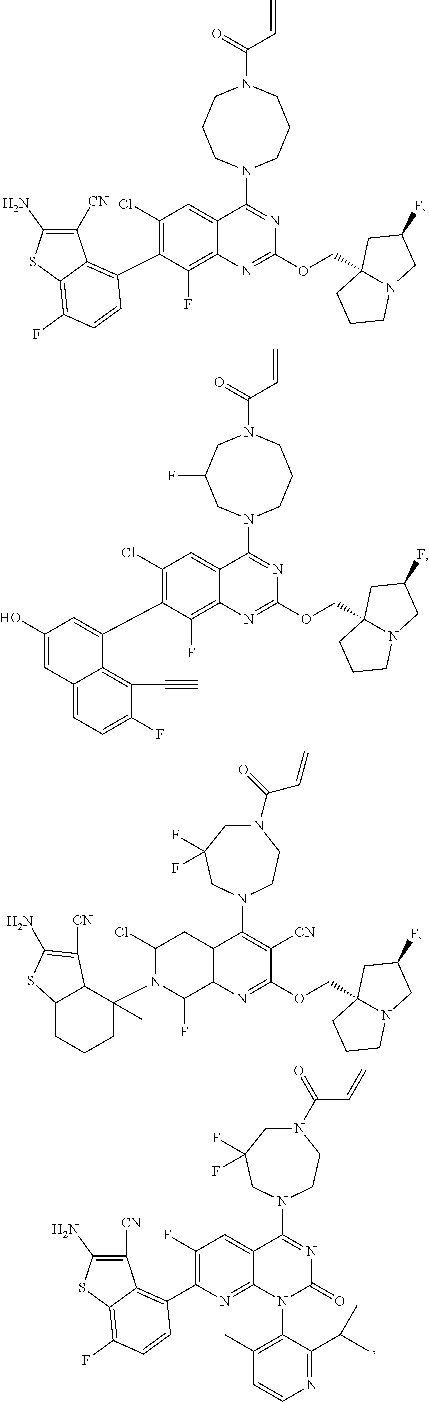

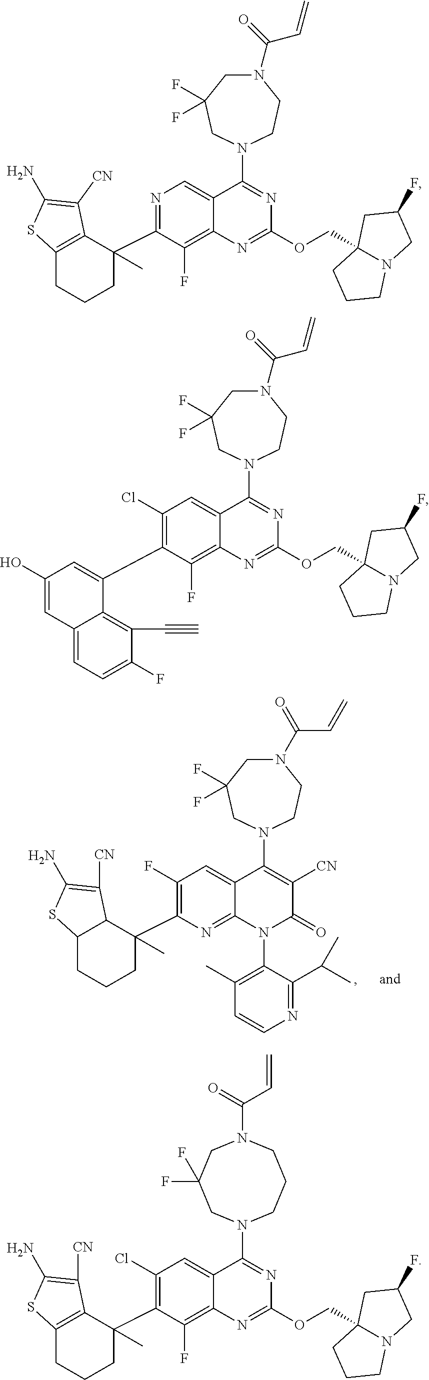

In embodiments, the subject compound is a compound described herein, including in the Example section below. It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. In some embodiments, the following synthetic methods may be utilized.

### General Synthetic Method 1

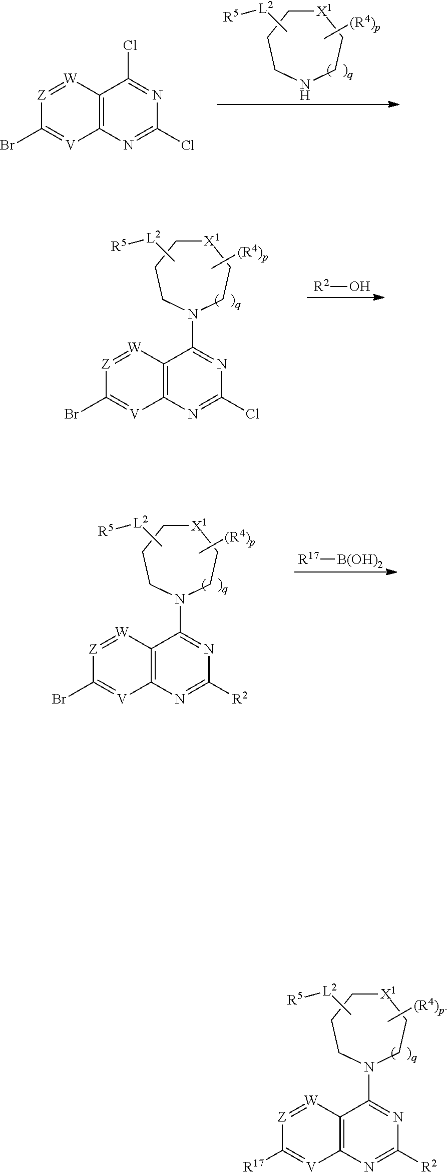

### General Synthetic Method 2

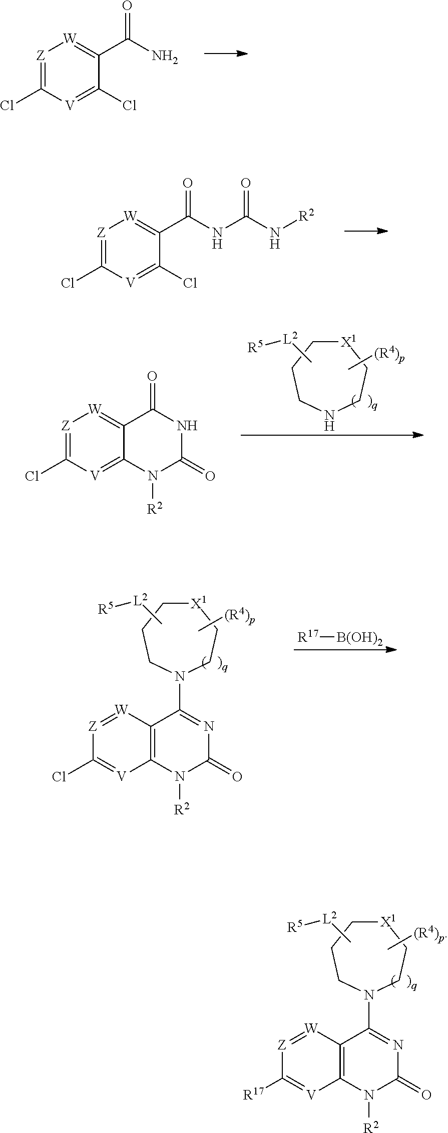

### General Synthetic Method 3

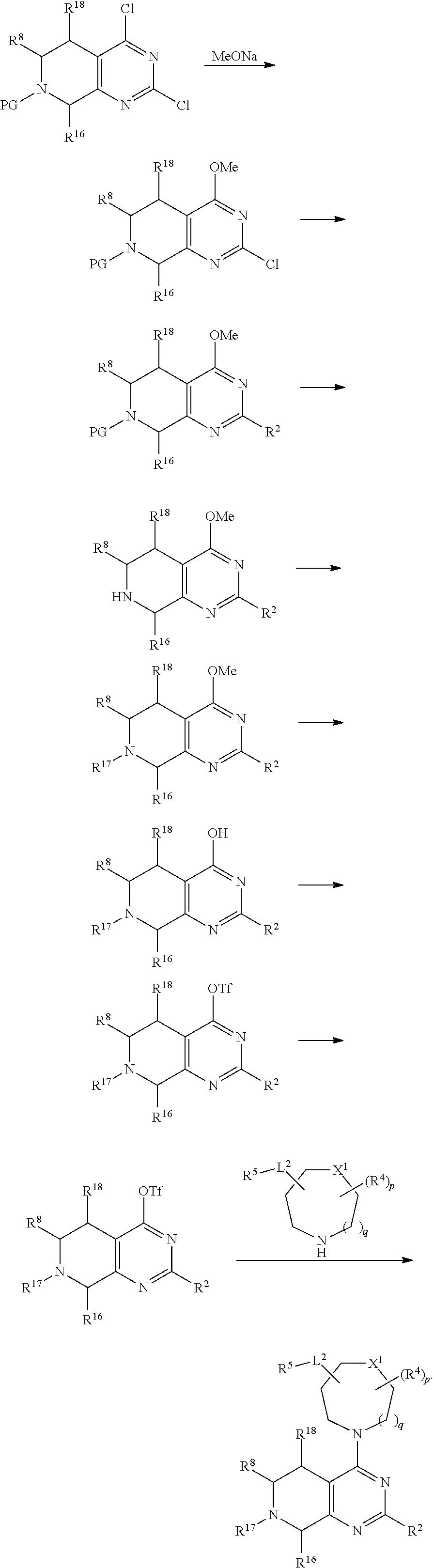

### General Synthetic Method 4

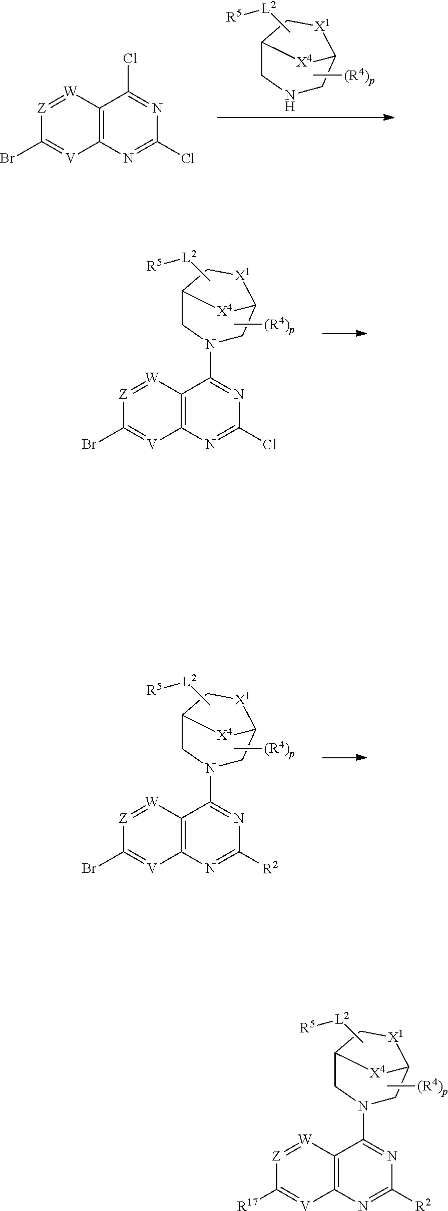

### General Synthetic Method 5:

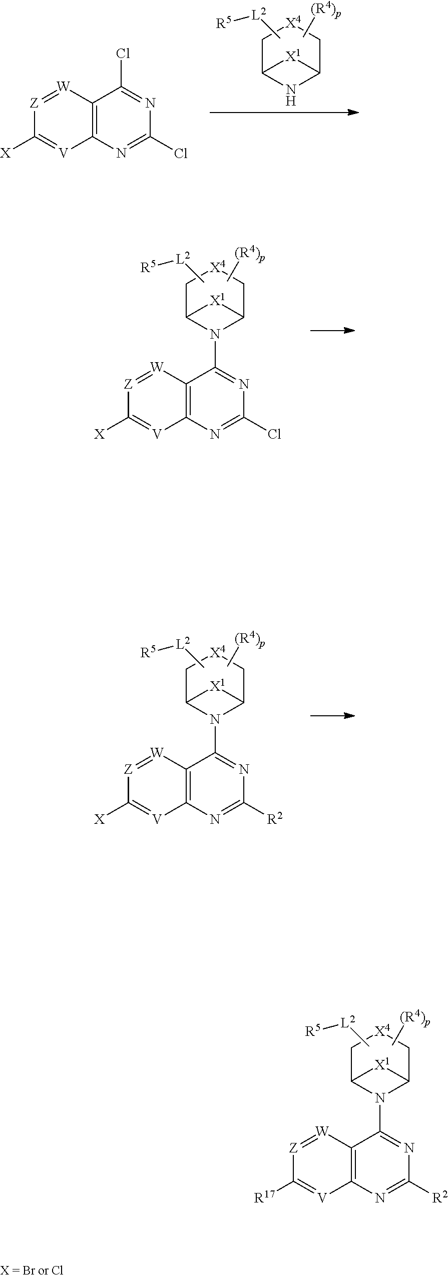

In some embodiments, the compounds of the present invention exhibit one or more functional characteristics disclosed herein. For example, a subject compound binds to a Ras protein, Kras protein or a mutant form thereof. In some embodiments, a subject compound binds specifically and also inhibits a Ras protein, Kras protein or a mutant form thereof. In some embodiments, a subject compound selectively inhibits a Kras mutant relative to a wildtype Kras. In some embodiment, a subject compound selectively inhibits KrasG12D and/or KrasG12V relative to wildtype Kras. In some embodiments, the IC50 of a subject compound (including those shown in Table 1) for a Kras mutant (e.g., including G12D) is less than about less than about 5 uM, less than about 1 uM, less than about 50n nM, less than about 10 nM, less than about 1 nM, less than about 0.5 nM, less than about 100 pM, or less than about 50 pM, as measured in an in vitro assay known in the art or exemplified herein.

In some embodiments, a subject compound of the present disclosure is capable of reducing Ras signaling output. Such reduction can be evidenced by one or more members of the following: (i) an increase in steady state level of GDP-bound Ras protein; (ii) a reduction of phosphorylated AKTs473, (iii) a reduction of phosphorylated ERKT202/y204, (iv) a reduction of phosphorylated S6S235/236, and (v) reduction (e.g., inhibition) of cell growth of Ras-driven tumor cells (e.g., those derived from a tumor cell line disclosed herein). In some cases, the reduction in Ras signaling output can be evidenced by two, three, four or all of (i)-(v) above.

### Methods

In some embodiments is a method of treating cancer in a subject in need thereof, comprising administering to the subject a therapeutically effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof. In some embodiments is a method of treating cancer in a subject in need thereof, comprising administering to the subject a therapeutically effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the cancer is a solid tumor or a hematological cancer. Non limiting examples of cancer that can be treated by any of the subject method utilizing a subject compound include prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. Non limiting examples of hematological cancer that can be treated by any subject method utilizing a subject compound disclosed herein include one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments, a subject method utilizing a subject compound disclosed herein is applied for treating chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

In some embodiments is a method of treating cancer in a subject in need thereof, comprising administering to the subject a therapeutically effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof. In some embodiments is a method of treating cancer in a subject in need thereof, comprising administering to the subject a therapeutically effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the cancer is a solid tumor or a hematological cancer. In some embodiments is a method of treating cancer in a subject in need thereof, comprising administering to the subject a therapeutically effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the cancer is a solid tumor. Non limiting examples of cancer that can be treated by any of the subject method utilizing a subject compound disclosed above include prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. Non limiting examples of hematological cancer that can be treated by any subject method utilizing a subject compound disclosed above include one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

In some embodiments is a method of treating cancer in a subject in need thereof, comprising administering to the subject a therapeutically effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof. In some embodiments is a method of treating cancer in a subject in need thereof, comprising administering to the subject a therapeutically effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the cancer is a solid tumor or a hematological cancer. Non limiting examples of cancer that can be treated by any of the subject method utilizing a subject compound disclosed above include prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the cancer is a hematological cancer. Non limiting examples of hematological cancer that can be treated by any subject method utilizing a subject compound disclosed above include one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the compound disclosed in, including compounds described in the “ADDITIONAL EMBODIMENTS” section, can be utilized in a method of treating cancer, modulating activity of a Ras protein, reducing Ras signaling output, or inhibiting activity of a KrasG12D mutant protein or other mutants of Ras protein

Any of the treatment methods disclosed herein can be administered alone or in combination or in conjunction with another therapy or another agent. By “combination” it is meant to include (a) formulating a subject composition containing a subject compound together with another agent, and (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiment, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof.

In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-1BB antibody (e.g., urelumab, PF-05082566), an anti-OX40 antibody (pogalizam favola lixizumab, PP-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies (a avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some embodiments, any of the compounds herein that is capable of binding a Ras protein (e.g., KRAS) to modulate activity of such Ras protein may be administered in combination or in conjunction with one or more pharmacologically active agents comprising (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY²⁹⁴⁰⁰², AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor (e.g., 6-(4-amino-4-methylpiperidin-1-yl)-3-(2,3-dichlorophenyl)pyrazin-2-amine), as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), or (32) an inhibitor of FLT3, HDAC, VEGFR, PDGFR, LCK, Bcr-Abl or AKT. In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L¹ antibody, anti-CLTA-4 antibody).

In some embodiments, any of the compounds herein that is capable of binding a Ras protein (e.g., KRAS) to modulate activity of such Ras protein may be administered in combination or in conjunction with one or more pharmacologically active agents comprising (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY²⁹⁴⁰⁰², AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of SHP pathway including SHIP2 inhibitor (e.g., 6-(4-amino-4-methylpiperidin-1-yl)-3-(2,3-dichlorophenyl)pyrazin-2-amine, as well as SHP1 inhibitors, or SHP2 inhibitors, or (33) an inhibitor of Src, FLT3, HDAC, VEGFR, PDGFR, LCK, Bcr-Abl or AKT. In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody).

In some embodiments, any of the compounds herein that is capable of binding a Ras protein (e.g., KRAS) to modulate activity of such Ras protein may be administered in combination or in conjunction with one or more pharmacologically active agents comprising an inhibitor of: (1) SOS1 or a mutant thereof (e.g., BAY-293, BI-1701963); (2) SHP2 or a mutant thereof (e.g., 6-(4-amino-4-methylpiperidin-1-yl)-3-(2,3-dichlorophenyl)pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., Sorafenib, Vemurafenib, Dabrafenib, Encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX⁰²¹⁸⁹, LY³²¹⁴⁹⁹⁶, ravoxertinib); (11) PI3K or a mutant thereof (e.g., Idelalisib, Copanlisib, Duvelisib, Alpelisib, Taselisib, Perifosine, Buparlisib, Umbralisib, NVP-BEZ²³⁵-AN); (12) MAPK or a mutant thereof (e.g., VX-745, VX-702, RO-4402257, SCIO-469, BIRB-796, SD-0006, PH-797804, AMG-548, LY²²²⁸⁸²⁰, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFR1, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R428, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY²⁹⁴⁰⁰², AZD5153, MT-1, MS645); (26) IGF1, IGF2, IGFIR, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In some embodiments, any of the compounds herein that is capable of binding a Ras protein (e.g., KRAS) to modulate activity of such Ras protein may be administered in combination or in conjunction with one or more pharmacologically active agents comprising (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY²⁹⁴⁰⁰², AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of SHP pathway including SHP2 inhibitor (e.g., 6-(4-amino-4-methylpiperidin-1-yl)-3-(2,3-dichlorophenyl)pyrazin-2-amine, as well as SHP1 inhibitors, or (33) an inhibitor of Src, FLT3, HDAC, VEGFR, PDGFR, LCK, Bcr-Abl or AKT or (34) an inhibitor of KrasG12C mutant (e.g., including but not limited to AMG510, MRTX⁸⁴⁹, and any covalent inhibitors binding to the cysteine residue 12 of Kras, the structures of these compounds are publicly known)(e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety),), (35) a SHC inhibitor (e.g., PP2, AID371185), (36) a GAB inhibitor (e.g., GAB-0001), (37) a GRB inhibitor, (38) a PI-3 kinase inhibitor (e.g., Idelalisib, Copanlisib, Duvelisib, Alpelisib, Taselisib, Perifosine, Buparlisib, Umbralisib, NVP-BEZ²³⁵-AN), (39) a MARPK inhibitor, (40) CDK4/6 (e.g., palbociclib, ribociclib, abemaciclib), or (41) MAPK inhibitor (e.g., VX-745, VX-702, RO-4402257, SCIO-469, BIRB-796, SD-0006, PH-797804, AMG-548, LY²²²⁸⁸²⁰, SB-681323, GW-856553, RWJ67657, BCT-197), or (42) an inhibitor of SHP pathway including SHP2 inhibitor (e.g., 6-(4-amino-4-methylpiperidin-1-yl)-3-(2,3-dichlorophenyl)pyrazin-2-amine, RMC-4630, TNO155

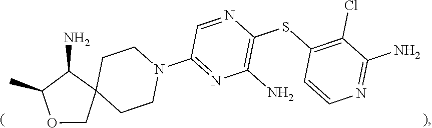 [SMILES: C[C@@H]1OCC2(CCN(c3cnc(Sc4ccnc(N)c4Cl)c(N)n3)CC2)[C@@H]1N]

### JAB-3068

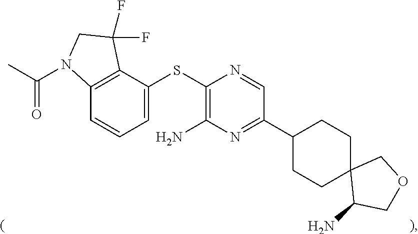 [SMILES: CC(=O)N1CC(F)(F)c2c(Sc3ncc(C4CCC5(CC4)COC[C@H]5N)nc3N)cccc21]

### IACS-13909/BBP-398

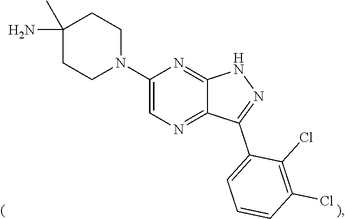 [SMILES: CC1(N)CCN(c2cnc3c(-c4cccc(Cl)c4Cl)n[nH]c3n2)CC1]

### SHP099

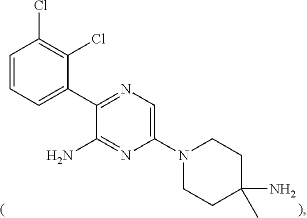 [SMILES: CC1(N)CCN(c2cnc(-c3cccc(Cl)c3Cl)c(N)n2)CC1]

### ERAS-601, and KMIC-4550

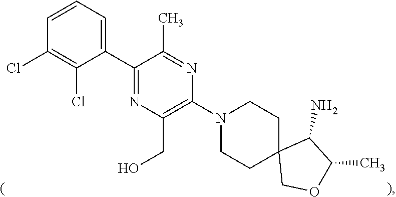 [SMILES: Cc1nc(N2CCC3(CC2)CO[C@@H](C)[C@H]3N)c(CO)nc1-c1cccc(Cl)c1Cl]

as well as SHP1 inhibitors. In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody). In some embodiments, any of the compounds herein that is capable of binding a Ras protein (e.g., KRAS) to modulate activity of such Ras protein may be administered in combination or in conjunction with one or more pharmacologically active agents comprising an inhibitor against one or more targets selected from the group of: MEK, epidermal growth factor receptor (EGFR), FGFR1, FGFR2, FGFR3, mitotic kinase, topoisomerase, ALK, c-MET, ErbB2, AXL, NTRK1, RET, A-Raf, B-Raf, C-Raf, ERK, MDM2, mTOR, BET, IGF1/2, IGF1-R, CDK9, SHIP1, SHIP2, SHP2, SRC, JAK, PARP, BTK, FLT3, HDAC, VEGFR, PDGFR, LCK, Bcr-Abl, AKT, KrasG12C mutant, and ROS1. Where desired, the additional agent can be an inhibitor against one or more targets selected from the group of: MEK, epidermal growth factor receptor (EGFR), FGFR1, FGFR2, FGFR3, mitotic kinase, topoisomerase, ALK, c-MET, ErbB2, AXL, NTRK1, RET, A-Raf, B-Raf, C-Raf, ERK, MDM2, mTOR, BET, IGF1/2, IGF1-R, CDK9, SHP2, SRC, JAK, PARP, BTK, FLT3, HDAC, VEGFR, PDGFR, LCK, Bcr-Abl, AKT, KrasG12C mutant, and ROS1. In some embodiments, any of the compounds herein that is capable of binding a Ras protein (e.g., KRAS, mutant Ras protein) to modulate activity of such Ras mutant (e.g., G12C, G12D, G12S, G12V, G13C, or G13D) may be administered in combination or in conjunction with one or more additional pharmacologically active agents comprising an inhibitor of SOS (e.g., SOS1, SOS2) or of mutants thereof. In embodiments, the additional pharmacologically active agent administered in combination or in conjunction with a compound described herein (e.g., compound capable of binding a Ras protein) is an inhibitor of SOS (e.g., SOS1, SOS2). In embodiments, the additional pharmacologically active agent administered in combination or in conjunction with a compound (e.g., compound capable of binding a Ras protein) described herein is an inhibitor of SOS (e.g., SOS1, SOS2). In embodiments, the additional pharmacologically active agent administered in combination or in conjunction with a compound (e.g., compound capable of binding a Ras protein) described herein is an inhibitor of SOS (e.g., SOS1, SOS2) selected from RMC-5845, BI-3406

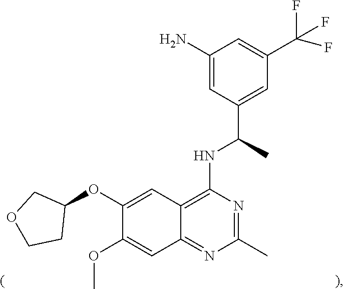 [SMILES: COc1cc2nc(C)nc(N[C@H](C)c3cc(N)cc(C(F)(F)F)c3)c2cc1O[C@H]1CCOC1]

### BI-1701963, and BAY 293

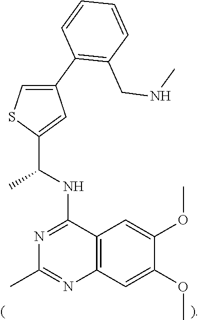

In embodiments, the additional pharmacologically active agent administered in combination or in conjunction with a compound described herein (e.g., compound capable of binding a Ras protein) is an inhibitor of SOS (e.g., SOS1, SOS2) described in WO2021092115, WO2018172250, WO2019201848, WO2019122129, WO2018115380, WO2021127429, WO2020180768, or WO2020180770, all of which are herein incorporated by reference in their entirety for all purposes.

In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody).

In some embodiments, any of the compounds described herein that is capable of binding a Ras protein (e.g., KRAS) may be administered in combination or in conjunction with one or more pharmacologically active agents comprising an inhibitor of: (1) SOS1 or a mutant thereof (e.g., RMC-5845, BI-3406, BAY-293, BI-1701963); (2) SHP2 or a mutant thereof (e.g., 6-(4-amino-4-methylpiperidin-1-yl)-3-(2,3-dichlorophenyl)pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., Sorafenib, Vemurafenib, Dabrafenib, Encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX⁰²¹⁸⁹, LY³²¹⁴⁹⁹⁶, ravoxertinib); (11) PI3K or a mutant thereof (e.g., Idelalisib, Copanlisib, Duvelisib, Alpelisib, Taselisib, Perifosine, Buparlisib, Umbralisib, NVP-BEZ²³⁵-AN); (12) MAPK or a mutant thereof (e.g., VX-745, VX-702, RO-4402257, SCIO-469, BIRB-796, SD-0006, PH-797804, AMG-548, LY²²²⁸⁸²⁰, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFR1, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R428, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY²⁹⁴⁰⁰², AZD5153, MT-1, MS645); (26) IGF1, IGF2, IGF1R, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In combination therapy, a compound provided herein and other anti-cancer agent(s) may be administered either simultaneously, concurrently or sequentially with no specific time limits, wherein such administration provides therapeutically effective levels of the two compounds in the body of the patient.

In some embodiments, the compound of the present disclosure and the other anti-cancer agent(s) are generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

An antibiotic can be administered to a subject as part of a therapeutic regime. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3^{rd} or 4^{th} generation, can be cephalosporin or a quinolone. An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin. In some cases, an antibiotic can be 1^{st} generation, 2^{nd} generation, 3^{rd} generation, 4^{th} generation, or 5^{th} generation. A first-generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3^{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole.

In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. It can be supplied in 500 mg tablets. Valacyclovir can be administered at a therapeutically effective amount.

In some cases, a treatment regime may be dosed according to a body weight of a subject. In subjects who are determined obese (BMI>35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases, a body surface area may be utilized to calculate a dosage. A body surface area (BSA) may be calculated by: BSA (m2)=√Height (cm)*Weight (kg)/3600.

In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (Ille-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (Ille-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, thereby modulating the activity of the Ras protein. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa- 1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein said modulating comprises inhibiting the Ras protein activity. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the Ras protein is a K-Ras protein. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb- 1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the Ras protein is a G12D or G12V mutant K-Ras. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the Ras protein is a G12D mutant K-Ras. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the Ras protein is a G12V mutant K-Ras. In some embodiments is a method of modulating activity of a Ras protein, comprising contacting a Ras protein with an effective amount of a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (Ille-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), or (Vc-2), or a pharmaceutically acceptable salt or solvate thereof, wherein the Ras protein is a mutant Ras protein (e.g., G12C, G12S, G12D, G13D, G13C, or G12V).

In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, thereby modulating the activity of the Ras protein. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein said modulating comprises inhibiting the Ras protein activity. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the Ras protein is a K-Ras protein. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the Ras protein is a G12D or G12V mutant K-Ras. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the Ras protein is a G12D mutant K-Ras. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the Ras protein is a G12V mutant K-Ras. In some embodiments is a method of modulating activity of a Ras protein, comprising contacting a Ras protein with an effective amount of a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein the Ras protein is a mutant Ras protein (e.g., G12C, G12S, G12D, G13D, G13C, or G12V).

In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, thereby modulating the activity of the Ras protein. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein said modulating comprises inhibiting the Ras protein activity. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the Ras protein is a K-Ras protein. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the Ras protein is a G12D or G12V mutant K-Ras. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the Ras protein is a G12D mutant K-Ras. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the Ras protein is a G12V mutant K-Ras. In some embodiments is a method of modulating activity of a Ras protein, comprising contacting a Ras protein with an effective amount of a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein the Ras protein is a mutant Ras protein (e.g., G12C, G12S, G12D, G13D, G13C, or G12V).

In some embodiments, provided is a method of reducing Ras signaling output in a cell by contacting the cell with a compound of the present disclosure. A reduction in Ras signalling can be evidenced by one or more members of the following: (i) an increase in steady state level of GDP-bound Ras protein; (ii) a reduction of phosphorylated AKTs473, (iii) a reduction of phosphorylated ERKT202/y204, (iv) a reduction of phosphorylated S6S235/236, and (v) reduction (e.g., inhibition) of cell growth of Ras-driven tumor cells (e.g., those derived from a tumor cell line). In some cases, the reduction in Ras signaling output can be evidenced by two, three, four or all of (i)-(v) above. In some embodiments, the reduction any one or more of (i)-(v) can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to control untreated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine (i.e. colon)), endometrium, stomach, hematopoietic tissue (e.g., lymphoid tissue), etc. Examples of the tumor cell line with a K-Ras mutation may include, but are not limited to, A549 (e.g., K-Ras G12S), AGS (e.g., K-Ras G12D), ASPC1 (e.g., K-Ras G12D), Calu-6 (e.g., K-Ras Q61K), CFPAC-1 (e.g., K-Ras G12V), CL40 (e.g., K-Ras G12D), COLO678 (e.g., K-Ras G12D), COR-L23 (e.g., K-Ras G12V), DAN-G (e.g., K-Ras G12V), GP2D (e.g., K-Ras G12D), GSU (e.g., K-Ras G12F), HCT116 (e.g., K-Ras G13D), HECIA (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCT1 (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANCI (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLUI (e.g., K-Ras G12D), SKM-1 (e.g., K-Ras K117N), SNU1 (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q61L), T3M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

In some embodiments is a Ras protein bound by a compound of Formula (I-1), (I-2), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof, wherein activity of said Ras protein is reduced as compared to a Ras protein unbound to said compound.

In some embodiments is a Ras protein bound by a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof, wherein activity of said Ras protein is reduced as compared to a Ras protein unbound to said compound.

In some embodiments is a Ras protein bound by a compound of Formula (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, wherein activity of said Ras protein is reduced as compared to a Ras protein unbound to said compound.

In some embodiments is a method of inhibiting activity of a KrasG12D mutant protein, comprising contacting the KrasG12D mutant protein with a compound of Formula (XI′-1), or a pharmaceutically acceptable salt or solvate thereof,

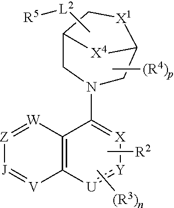

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X⁴ is selected from C(O), C═N—OR⁴, C—NNR⁴R⁶, N(R¹), N(R⁶), S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N;
Z is N or C(R⁸), wherein at least one of U, X, and Z is not N;
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰%;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²)(R²³), —C(O)OR²², —C(O)N(R²)(R²³), —C(O)C(O)N(R²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In an aspect is a method of inhibiting activity of a KrasG12D mutant protein, comprising contacting the KrasG12D mutant protein with a compound of Formula (XXI′-1), or a pharmaceutically acceptable salt or solvate thereof,

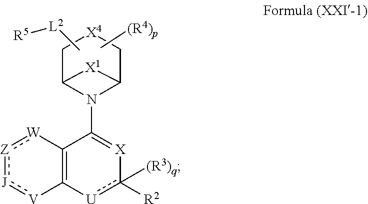

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁴)C(R⁴)(R⁶), C(R⁴)(R⁶), C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁴)OC(R⁴)(R⁶), C(R⁴)(R⁶)S, C(R⁴)(R⁶)SC(R⁴)(R⁶), C(R⁴)(R⁴), S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁴)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ,
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d},
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12b};
L³ is selected from C₁-C₆alkyl, C₂₋₆alkenyl, C₂₋₆alkynyl, —O—, —N(R¹⁴)—, and —S—;
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C_{1- 9}heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is a method of inhibiting activity of a KrasG12D mutant protein, comprising contacting the KrasG12D mutant protein with a compound of Formula (XXII′-1), or a pharmaceutically acceptable salt or solvate thereof,

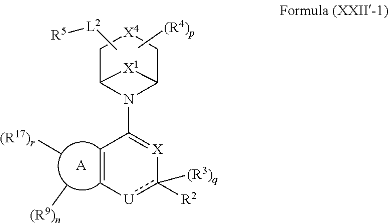

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C—N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁴), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁴)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R¹⁸)O, C(R^{1f})(R¹⁸)N(R^{1c}), and C(R¹⁶)(R^{1g});
each R^{1c}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d}.

R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In some embodiments is a method of inhibiting activity of a KrasG12D mutant protein, comprising contacting the KrasG12D mutant protein with a compound of Formula (XI′-2), or a pharmaceutically acceptable salt or solvate thereof,

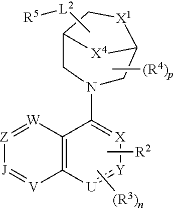

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—; X⁴ is selected from C(O), C═N—OR⁴, C═NNR⁴R⁶, N(R¹), N(R⁶), S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N;
Z is N or C(R⁸), wherein at least one of U, X, and Z is not N; V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

In an aspect is a method of inhibiting activity of a KrasG12D mutant protein, comprising contacting the KrasG12D mutant protein with a compound of Formula (XXI′-2), or a pharmaceutically acceptable salt or solvate thereof,

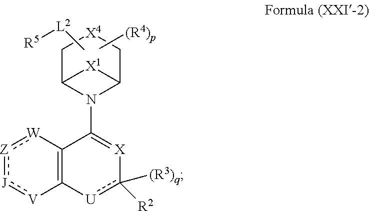

wherein:
X is C(R³) or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁴)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁴)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁴)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a}.
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R¹)(R¹⁸)O, C(R¹)(R^{1g})N(R^{1c}), and C(R^{1f})(R¹⁸);
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ,
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d},
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12b};
L³ is selected from C₁-C₆alkyl, C₂₋₆alkenyl, C₂₋₆alkynyl, —O—, —N(R¹⁴)—, and —S—;
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C_{1- 9}heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

In an aspect is a method of inhibiting activity of a KrasG12D mutant protein, comprising contacting the KrasG12D mutant protein with a compound of Formula (XXII′-2), or a pharmaceutically acceptable salt or solvate thereof,

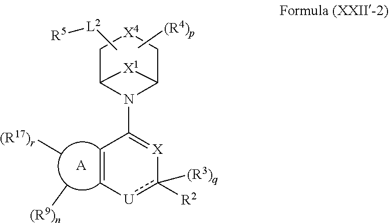

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁴)N(R⁶), C(R⁴)(R⁶)O, C(R⁴)(R⁶)OC(R⁴)(R⁴), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁴)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X⁴ is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R¹⁸)O, C(R¹)(R^{1g})N(R^{1c}), and C(R^{1f})(R¹⁸);
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

### Pharmaceutical Compositions and Methods of Administration

The compounds of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, described herein are administered to subjects in a biologically compatible form suitable for administration to treat or prevent diseases, disorders or conditions. Administration of the compounds described herein can be in any pharmacological form including a therapeutically effective amount of a compound of Formula (I-1), (1-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa-1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), (XXIV-2), or a pharmaceutically acceptable salt or solvate thereof, alone or in combination with a pharmaceutically acceptable carrier.

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments is a pharmaceutical composition comprising a pharmaceutically acceptable excipient and a compound of Formula (I-1), (I-2), (I′), (I″), (Ia), (Ib), (Ic), (Id), (Ie), (If), (Ig), (II), (IIa), (IIb), (IIc′), (IIc), (IId′), (IId), (IIe), (IIf), (IIg), (IIh), (IIi), (IIj), (IIk), (IIm), (IIIa), (IIIb), (IIIc), (IIId), (IIIe), (IIIf), (IIIg), (IIIh), (IIIi), (IVa), (IVb), (IVc), (Va), (Vb), or (Vc), or a pharmaceutically acceptable salt or solvate thereof. In some embodiments is a pharmaceutical composition comprising a pharmaceutically acceptable excipient and a compound of Formula (I-1), or a pharmaceutically acceptable salt or solvate thereof. In some embodiments is a pharmaceutical composition comprising a pharmaceutically acceptable excipient and a compound of Formula (I-2), or a pharmaceutically acceptable salt or solvate thereof. In some embodiments is a pharmaceutical composition comprising a pharmaceutically acceptable excipient and a compound of Formula (I′), or a pharmaceutically acceptable salt or solvate thereof. In some embodiments is a pharmaceutical composition comprising a pharmaceutically acceptable excipient and a compound of Formula (I″), or a pharmaceutically acceptable salt or solvate thereof. In some embodiments is a pharmaceutical composition comprising a pharmaceutically acceptable excipient and a compound of Formula (Ia), or a pharmaceutically acceptable salt or solvate thereof. In some embodiments is a pharmaceutical composition comprising a pharmaceutically acceptable excipient and a compound of Formula (Ib), or a pharmaceutically acceptable salt or solvate thereof. In some embodiments is a pharmaceutical composition comprising a pharmaceutically acceptable excipient and a compound of Formula (Ic), or a pharmaceutically acceptable salt or solvate thereof. In some embodiments is a pharmaceutical composition comprising a pharmaceutically acceptable excipient and a compound of Formula (Id), or a pharmaceutically acceptable salt or solvate thereof. In some embodiments is a pharmaceutical composition comprising a pharmaceutically acceptable excipient and a compound of Formula (Ie), or a pharmaceutically acceptable salt or solvate thereof. In some embodiments is a pharmaceutical composition comprising a pharmaceutically acceptable excipient and a compound of Formula (If), or a pharmaceutically acceptable salt or solvate thereof. In some embodiments is a pharmaceutical composition comprising a pharmaceutically acceptable excipient and a compound of Formula (Ig), or a pharmaceutically acceptable salt or solvate thereof. In some embodiments is a pharmaceutical composition comprising a pharmaceutically acceptable excipient and a compound of Formula (II), or a pharmaceutically acceptable salt or solvate thereof. In some embodiments is a pharmaceutical composition comprising a pharmaceutically acceptable excipient and a compound of Formula (IIa), or a pharmaceutically acceptable salt or solvate thereof. In some embodiments is a pharmaceutical composition comprising a pharmaceutically acceptable excipient and a compound of Formula (IIb), or a pharmaceutically acceptable salt or solvate thereof. In some embodiments is a pharmaceutical composition comprising a pharmaceutically acceptable excipient and a compound of Formula (IIc′), or a pharmaceutically acceptable salt or solvate thereof. In some embodiments is a pharmaceutical composition comprising a pharmaceutically acceptable excipient and a compound of Formula (IIc), or a pharmaceutically acceptable salt or solvate thereof. In some embodiments is a pharmaceutical composition comprising a pharmaceutically acceptable excipient and a compound of Formula (IId′), or a pharmaceutically acceptable salt or solvate thereof. In some embodiments is a pharmaceutical composition comprising a pharmaceutically acceptable excipient and a compound of Formula (IId), or a pharmaceutically acceptable salt or solvate thereof. In some embodiments is a pharmaceutical composition comprising a pharmaceutically acceptable excipient and a compound of Formula (IIe), or a pharmaceutically acceptable salt or solvate thereof. In some embodiments is a pharmaceutical composition comprising a pharmaceutically acceptable excipient and a compound of Formula (IIf), or a pharmaceutically acceptable salt or solvate thereof. In some embodiments is a pharmaceutical composition comprising a pharmaceutically acceptable excipient and a compound of Formula (IIg), or a pharmaceutically acceptable salt or solvate thereof. In some embodiments is a pharmaceutical composition comprising a pharmaceutically acceptable excipient and a compound of Formula (IIh), or a pharmaceutically acceptable salt or solvate thereof. In some embodiments is a pharmaceutical composition comprising a pharmaceutically acceptable excipient and a compound of Formula (IIi), or a pharmaceutically acceptable salt or solvate thereof. In some embodiments is a pharmaceutical composition comprising a pharmaceutically acceptable excipient and a compound of Formula (IIj), or a pharmaceutically acceptable salt or solvate thereof. In some embodiments is a pharmaceutical composition comprising a pharmaceutically acceptable excipient and a compound of Formula (IIk), or a pharmaceutically acceptable salt or solvate thereof. In some embodiments is a pharmaceutical composition comprising a pharmaceutically acceptable excipient and a compound of Formula (IIm), or a pharmaceutically acceptable salt or solvate thereof. In some embodiments is a pharmaceutical composition comprising a pharmaceutically acceptable excipient and a compound of Formula (IIIa), or a pharmaceutically acceptable salt or solvate thereof. In some embodiments is a pharmaceutical composition comprising a pharmaceutically acceptable excipient and a compound of Formula (IIIb), or a pharmaceutically acceptable salt or solvate thereof. In some embodiments is a pharmaceutical composition comprising a pharmaceutically acceptable excipient and a compound of Formula (IIIc), or a pharmaceutically acceptable salt or solvate thereof. In some embodiments is a pharmaceutical composition comprising a pharmaceutically acceptable excipient and a compound of Formula (IIId), or a pharmaceutically acceptable salt or solvate thereof. In some embodiments is a pharmaceutical composition comprising a pharmaceutically acceptable excipient and a compound of Formula (IIIe), or a pharmaceutically acceptable salt or solvate thereof. In some embodiments is a pharmaceutical composition comprising a pharmaceutically acceptable excipient and a compound of Formula (IIIf), or a pharmaceutically acceptable salt or solvate thereof. In some embodiments is a pharmaceutical composition comprising a pharmaceutically acceptable excipient and a compound of Formula (IIIg), or a pharmaceutically acceptable salt or solvate thereof. In some embodiments is a pharmaceutical composition comprising a pharmaceutically acceptable excipient and a compound of Formula (IIIh), or a pharmaceutically acceptable salt or solvate thereof. In some embodiments is a pharmaceutical composition comprising a pharmaceutically acceptable excipient and a compound of Formula (IIIi), or a pharmaceutically acceptable salt or solvate thereof. In some embodiments is a pharmaceutical composition comprising a pharmaceutically acceptable excipient and a compound of Formula (IVa), or a pharmaceutically acceptable salt or solvate thereof. In some embodiments is a pharmaceutical composition comprising a pharmaceutically acceptable excipient and a compound of Formula (IVb), or a pharmaceutically acceptable salt or solvate thereof. In some embodiments is a pharmaceutical composition comprising a pharmaceutically acceptable excipient and a compound of Formula (IVc), or a pharmaceutically acceptable salt or solvate thereof. In some embodiments is a pharmaceutical composition comprising a pharmaceutically acceptable excipient and a compound of Formula (Va), or a pharmaceutically acceptable salt or solvate thereof. In some embodiments is a pharmaceutical composition comprising a pharmaceutically acceptable excipient and a compound of Formula (Vb), or a pharmaceutically acceptable salt or solvate thereof. In some embodiments is a pharmaceutical composition comprising a pharmaceutically acceptable excipient and a compound of Formula (Vc), or a pharmaceutically acceptable salt or solvate thereof. In some embodiments is a pharmaceutical composition comprising a pharmaceutically acceptable excipient and a compound of Formula (XI-1), (XI-2), (XIa), (XIb), (XIc), (XId), (XIe), (XIf), (XII-1), (XII-2), (XIIa), (XIIb), (XIIc), or (XIId), or a pharmaceutically acceptable salt or solvate thereof. In some embodiments is a pharmaceutical composition comprising a pharmaceutically acceptable excipient and a compound of Formula (XI), or a pharmaceutically acceptable salt or solvate thereof. In some embodiments is a pharmaceutical composition comprising a pharmaceutically acceptable excipient and a compound of Formula (XIa), or a pharmaceutically acceptable salt or solvate thereof. In some embodiments is a pharmaceutical composition comprising a pharmaceutically acceptable excipient and a compound of Formula (XIb), or a pharmaceutically acceptable salt or solvate thereof. In some embodiments is a pharmaceutical composition comprising a pharmaceutically acceptable excipient and a compound of Formula (XIc), or a pharmaceutically acceptable salt or solvate thereof. In some embodiments is a pharmaceutical composition comprising a pharmaceutically acceptable excipient and a compound of Formula (XId), or a pharmaceutically acceptable salt or solvate thereof. In some embodiments is a pharmaceutical composition comprising a pharmaceutically acceptable excipient and a compound of Formula (XIe), or a pharmaceutically acceptable salt or solvate thereof. In some embodiments is a pharmaceutical composition comprising a pharmaceutically acceptable excipient and a compound of Formula (XIf), or a pharmaceutically acceptable salt or solvate thereof. In some embodiments is a pharmaceutical composition comprising a pharmaceutically acceptable excipient and a compound of Formula (XII), or a pharmaceutically acceptable salt or solvate thereof. In some embodiments is a pharmaceutical composition comprising a pharmaceutically acceptable excipient and a compound of Formula (XIIa), or a pharmaceutically acceptable salt or solvate thereof. In some embodiments is a pharmaceutical composition comprising a pharmaceutically acceptable excipient and a compound of Formula (XIIb), or a pharmaceutically acceptable salt or solvate thereof. In some embodiments is a pharmaceutical composition comprising a pharmaceutically acceptable excipient and a compound of Formula (XIIc), or a pharmaceutically acceptable salt or solvate thereof. In some embodiments is a pharmaceutical composition comprising a pharmaceutically acceptable excipient and a compound of Formula (XIId), or a pharmaceutically acceptable salt or solvate thereof. In some embodiments is a pharmaceutical composition comprising a pharmaceutically acceptable excipient and a compound of Formula (XXI), (XXIa), (XXIb), (XXIc), (XXII), (XXIIa), (XXIIb), (XXIIc), (XXIId), (XXIIe), (XXIIf), (XXIIg), (XXIII), or (XXIV), or a pharmaceutically acceptable salt or solvate thereof. In some embodiments is a pharmaceutical composition comprising a pharmaceutically acceptable excipient and a compound of Formula (XXI), or a pharmaceutically acceptable salt or solvate thereof. In some embodiments is a pharmaceutical composition comprising a pharmaceutically acceptable excipient and a compound of Formula (XXIa), or a pharmaceutically acceptable salt or solvate thereof. In some embodiments is a pharmaceutical composition comprising a pharmaceutically acceptable excipient and a compound of Formula (XXIb), or a pharmaceutically acceptable salt or solvate thereof. In some embodiments is a pharmaceutical composition comprising a pharmaceutically acceptable excipient and a compound of Formula (XXIc), or a pharmaceutically acceptable salt or solvate thereof. In some embodiments is a pharmaceutical composition comprising a pharmaceutically acceptable excipient and a compound of Formula (XXII), or a pharmaceutically acceptable salt or solvate thereof. In some embodiments is a pharmaceutical composition comprising a pharmaceutically acceptable excipient and a compound of Formula (XXIIa), or a pharmaceutically acceptable salt or solvate thereof. In some embodiments is a pharmaceutical composition comprising a pharmaceutically acceptable excipient and a compound of Formula (XXIIb), or a pharmaceutically acceptable salt or solvate thereof. In some embodiments is a pharmaceutical composition comprising a pharmaceutically acceptable excipient and a compound of Formula (XXIIc), or a pharmaceutically acceptable salt or solvate thereof. In some embodiments is a pharmaceutical composition comprising a pharmaceutically acceptable excipient and a compound of Formula (XXIId), or a pharmaceutically acceptable salt or solvate thereof. In some embodiments is a pharmaceutical composition comprising a pharmaceutically acceptable excipient and a compound of Formula (XXIIe), or a pharmaceutically acceptable salt or solvate thereof. In some embodiments is a pharmaceutical composition comprising a pharmaceutically acceptable excipient and a compound of Formula (XXIIf), or a pharmaceutically acceptable salt or solvate thereof. In some embodiments is a pharmaceutical composition comprising a pharmaceutically acceptable excipient and a compound of Formula (XXIIg), or a pharmaceutically acceptable salt or solvate thereof. In some embodiments is a pharmaceutical composition comprising a pharmaceutically acceptable excipient and a compound of Formula (XXIII), or a pharmaceutically acceptable salt or solvate thereof. In some embodiments is a pharmaceutical composition comprising a pharmaceutically acceptable excipient and a compound of Formula (XXIV), or a pharmaceutically acceptable salt or solvate thereof.

In some embodiments is a pharmaceutical composition comprising a pharmaceutically acceptable excipient and a compound of Formula (I-1), (I-2), (I′), (I″-1), (I″-2), (Ia), (Ib), (Ic), (Id), (Ie), (If), (Ig), (II-1), (II-2), (IIa), (IIb), (IIc′), (IIc), (IId′), (IId), (IIe), (IIf), (IIg), (IIh), (IIi), (IIj), (IIk), (IIm), (IIIa-1), (IIIb-1), (IIIc-1), (IIId-1), (IIIe-1), (IIIf-1), (IIIg-1), (IIIh-1), (IIIi-1), (IIIa-2), (IIIb-2), (IIIc-2), (IIId-2), (IIIe-2), (IIIf-2), (IIIg-2), (IIIh-2), (IIIi-2), (IIIa-3), (IIIb-3), (IIIc-3), (IIId-3), (IIIe-3), (IIIf-3), (IIIg-3), (IIIh-3), (IIIi-3), (IIIa-4), (IIIb-4), (IIIc-4), (IIId-4), (IIIe-4), (IIIf-4), (IIIg-4), (IIIh-4), (IIIi-4), (IVa- 1), (IVb-1), (IVc-1), (IVa-2), (IVb-2), (IVc-2), (Va-1), (Vb-1), (Vc-1), (Va-2), (Vb-2), (Vc-2), (XI-1), (XI-2), (XIa), (XIb), (XIc), (XId), (XIe), (XIf), (XII-1), (XII-2), (XIIa), (XIIb), (XIIc), (XIId), (XXI-1), (XXI-2), (XXIa), (XXIb), (XXIc), (XXII-1), (XXII-2), (XXIIa-1), (XXIIa-2), (XXIIb-1), (XXIIb-2), (XXIIc), (XXIId), (XXIIe), (XXIIf), (XXIIg), (XXIII-1), (XXIII-2), (XXIV-1), or (XXIV-2), or a pharmaceutically acceptable salt or solvate thereof. In some embodiments is a pharmaceutical composition comprising a pharmaceutically acceptable excipient and a compound of Formula (I-1), or a pharmaceutically acceptable salt or solvate thereof. In some embodiments is a pharmaceutical composition comprising a pharmaceutically acceptable excipient and a compound of Formula (I-2), or a pharmaceutically acceptable salt or solvate thereof. In some embodiments is a pharmaceutical composition comprising a pharmaceutically acceptable excipient and a compound of Formula (I′), or a pharmaceutically acceptable salt or solvate thereof. In some embodiments is a pharmaceutical composition comprising a pharmaceutically acceptable excipient and a compound of Formula (I″-1), or a pharmaceutically acceptable salt or solvate thereof. In some embodiments is a pharmaceutical composition comprising a pharmaceutically acceptable excipient and a compound of Formula (I″-2), or a pharmaceutically acceptable salt or solvate thereof. In some embodiments is a pharmaceutical composition comprising a pharmaceutically acceptable excipient and a compound of Formula (Ia), or a pharmaceutically acceptable salt or solvate thereof. In some embodiments is a pharmaceutical composition comprising a pharmaceutically acceptable excipient and a compound of Formula (Ib), or a pharmaceutically acceptable salt or solvate thereof. In some embodiments is a pharmaceutical composition comprising a pharmaceutically acceptable excipient and a compound of Formula (Ic), or a pharmaceutically acceptable salt or solvate thereof. In some embodiments is a pharmaceutical composition comprising a pharmaceutically acceptable excipient and a compound of Formula (Id), or a pharmaceutically acceptable salt or solvate thereof. In some embodiments is a pharmaceutical composition comprising a pharmaceutically acceptable excipient and a compound of Formula (Ie), or a pharmaceutically acceptable salt or solvate thereof. In some embodiments is a pharmaceutical composition comprising a pharmaceutically acceptable excipient and a compound of Formula (If), or a pharmaceutically acceptable salt or solvate thereof. In some embodiments is a pharmaceutical composition comprising a pharmaceutically acceptable excipient and a compound of Formula (Ig), or a pharmaceutically acceptable salt or solvate thereof. In some embodiments is a pharmaceutical composition comprising a pharmaceutically acceptable excipient and a compound of Formula (II-1), or a pharmaceutically acceptable salt or solvate thereof. In some embodiments is a pharmaceutical composition comprising a pharmaceutically acceptable excipient and a compound of Formula (II-2), or a pharmaceutically acceptable salt or solvate thereof. In some embodiments is a pharmaceutical composition comprising a pharmaceutically acceptable excipient and a compound of Formula (IIa), or a pharmaceutically acceptable salt or solvate thereof. In some embodiments is a pharmaceutical composition comprising a pharmaceutically acceptable excipient and a compound of Formula (IIb), or a pharmaceutically acceptable salt or solvate thereof. In some embodiments is a pharmaceutical composition comprising a pharmaceutically acceptable excipient and a compound of Formula (IIc′), or a pharmaceutically acceptable salt or solvate thereof. In some embodiments is a pharmaceutical composition comprising a pharmaceutically acceptable excipient and a compound of Formula (IIc), or a pharmaceutically acceptable salt or solvate thereof. In some embodiments is a pharmaceutical composition comprising a pharmaceutically acceptable excipient and a compound of Formula (IId′), or a pharmaceutically acceptable salt or solvate thereof. In some embodiments is a pharmaceutical composition comprising a pharmaceutically acceptable excipient and a compound of Formula (IId), or a pharmaceutically acceptable salt or solvate thereof. In some embodiments is a pharmaceutical composition comprising a pharmaceutically acceptable excipient and a compound of Formula (IIe), or a pharmaceutically acceptable salt or solvate thereof. In some embodiments is a pharmaceutical composition comprising a pharmaceutically acceptable excipient and a compound of Formula (IIf), or a pharmaceutically acceptable salt or solvate thereof. In some embodiments is a pharmaceutical composition comprising a pharmaceutically acceptable excipient and a compound of Formula (IIg), or a pharmaceutically acceptable salt or solvate thereof. In some embodiments is a pharmaceutical composition comprising a pharmaceutically acceptable excipient and a compound of Formula (IIh), or a pharmaceutically acceptable salt or solvate thereof. In some embodiments is a pharmaceutical composition comprising a pharmaceutically acceptable excipient and a compound of Formula (IIi), or a pharmaceutically acceptable salt or solvate thereof. In some embodiments is a pharmaceutical composition comprising a pharmaceutically acceptable excipient and a compound of Formula (IIj), or a pharmaceutically acceptable salt or solvate thereof. In some embodiments is a pharmaceutical composition comprising a pharmaceutically acceptable excipient and a compound of Formula (IIk), or a pharmaceutically acceptable salt or solvate thereof. In some embodiments is a pharmaceutical composition comprising a pharmaceutically acceptable excipient and a compound of Formula (IIm), or a pharmaceutically acceptable salt or solvate thereof. In some embodiments is a pharmaceutical composition comprising a pharmaceutically acceptable excipient and a compound of Formula (IIIa-1), or a pharmaceutically acceptable salt or solvate thereof. In some embodiments is a pharmaceutical composition comprising a pharmaceutically acceptable excipient and a compound of Formula (IIIb-1), or a pharmaceutically acceptable salt or solvate thereof. In some embodiments is a pharmaceutical composition comprising a pharmaceutically acceptable excipient and a compound of Formula (IIIc-1), or a pharmaceutically acceptable salt or solvate thereof. In some embodiments is a pharmaceutical composition comprising a pharmaceutically acceptable excipient and a compound of Formula (IIId-1), or a pharmaceutically acceptable salt or solvate thereof. In some embodiments is a pharmaceutical composition comprising a pharmaceutically acceptable excipient and a compound of Formula (IIIe-1), or a pharmaceutically acceptable salt or solvate thereof. In some embodiments is a pharmaceutical composition comprising a pharmaceutically acceptable excipient and a compound of Formula (IIIf-1), or a pharmaceutically acceptable salt or solvate thereof. In some embodiments is a pharmaceutical composition comprising a pharmaceutically acceptable excipient and a compound of Formula (IIIg-1), or a pharmaceutically acceptable salt or solvate thereof. In some embodiments is a pharmaceutical composition comprising a pharmaceutically acceptable excipient and a compound of Formula (IIIh-1), or a pharmaceutically acceptable salt or solvate thereof. In some embodiments is a pharmaceutical composition comprising a pharmaceutically acceptable excipient and a compound of Formula (IIIi-1), or a pharmaceutically acceptable salt or solvate thereof. In some embodiments is a pharmaceutical composition comprising a pharmaceutically acceptable excipient and a compound of Formula (IIIa-2), or a pharmaceutically acceptable salt or solvate thereof. In some embodiments is a pharmaceutical composition comprising a pharmaceutically acceptable excipient and a compound of Formula (IIIb-2), or a pharmaceutically acceptable salt or solvate thereof. In some embodiments is a pharmaceutical composition comprising a pharmaceutically acceptable excipient and a compound of Formula (IIIc-2), or a pharmaceutically acceptable salt or solvate thereof. In some embodiments is a pharmaceutical composition comprising a pharmaceutically acceptable excipient and a compound of Formula (IIId-2), or a pharmaceutically acceptable salt or solvate thereof. In some embodiments is a pharmaceutical composition comprising a pharmaceutically acceptable excipient and a compound of Formula (IIIe-2), or a pharmaceutically acceptable salt or solvate thereof. In some embodiments is a pharmaceutical composition comprising a pharmaceutically acceptable excipient and a compound of Formula (IIIf-2), or a pharmaceutically acceptable salt or solvate thereof. In some embodiments is a pharmaceutical composition comprising a pharmaceutically acceptable excipient and a compound of Formula (IIIg-2), or a pharmaceutically acceptable salt or solvate thereof. In some embodiments is a pharmaceutical composition comprising a pharmaceutically acceptable excipient and a compound of Formula (IIIh-2), or a pharmaceutically acceptable salt or solvate thereof. In some embodiments is a pharmaceutical composition comprising a pharmaceutically acceptable excipient and a compound of Formula (IIIi-2), or a pharmaceutically acceptable salt or solvate thereof. In some embodiments is a pharmaceutical composition comprising a pharmaceutically acceptable excipient and a compound of Formula (IIIa-3), or a pharmaceutically acceptable salt or solvate thereof. In some embodiments is a pharmaceutical composition comprising a pharmaceutically acceptable excipient and a compound of Formula (IIIb-3), or a pharmaceutically acceptable salt or solvate thereof. In some embodiments is a pharmaceutical composition comprising a pharmaceutically acceptable excipient and a compound of Formula (IIIc-3), or a pharmaceutically acceptable salt or solvate thereof. In some embodiments is a pharmaceutical composition comprising a pharmaceutically acceptable excipient and a compound of Formula (IIId-3), or a pharmaceutically acceptable salt or solvate thereof. In some embodiments is a pharmaceutical composition comprising a pharmaceutically acceptable excipient and a compound of Formula (IIIe-3), or a pharmaceutically acceptable salt or solvate thereof. In some embodiments is a pharmaceutical composition comprising a pharmaceutically acceptable excipient and a compound of Formula (IIIf-3), or a pharmaceutically acceptable salt or solvate thereof. In some embodiments is a pharmaceutical composition comprising a pharmaceutically acceptable excipient and a compound of Formula (IIIg-3), or a pharmaceutically acceptable salt or solvate thereof. In some embodiments is a pharmaceutical composition comprising a pharmaceutically acceptable excipient and a compound of Formula (IIIh-3), or a pharmaceutically acceptable salt or solvate thereof. In some embodiments is a pharmaceutical composition comprising a pharmaceutically acceptable excipient and a compound of Formula (IIIi-3), or a pharmaceutically acceptable salt or solvate thereof. In some embodiments is a pharmaceutical composition comprising a pharmaceutically acceptable excipient and a compound of Formula (IIIa-4), or a pharmaceutically acceptable salt or solvate thereof. In some embodiments is a pharmaceutical composition comprising a pharmaceutically acceptable excipient and a compound of Formula (IIIb-4), or a pharmaceutically acceptable salt or solvate thereof. In some embodiments is a pharmaceutical composition comprising a pharmaceutically acceptable excipient and a compound of Formula (IIIc-4), or a pharmaceutically acceptable salt or solvate thereof. In some embodiments is a pharmaceutical composition comprising a pharmaceutically acceptable excipient and a compound of Formula (IIId-4), or a pharmaceutically acceptable salt or solvate thereof. In some embodiments is a pharmaceutical composition comprising a pharmaceutically acceptable excipient and a compound of Formula (IIIe-4), or a pharmaceutically acceptable salt or solvate thereof. In some embodiments is a pharmaceutical composition comprising a pharmaceutically acceptable excipient and a compound of Formula (IIIf-4), or a pharmaceutically acceptable salt or solvate thereof. In some embodiments is a pharmaceutical composition comprising a pharmaceutically acceptable excipient and a compound of Formula (IIIg-4), or a pharmaceutically acceptable salt or solvate thereof. In some embodiments is a pharmaceutical composition comprising a pharmaceutically acceptable excipient and a compound of Formula (IIIh-4), or a pharmaceutically acceptable salt or solvate thereof. In some embodiments is a pharmaceutical composition comprising a pharmaceutically acceptable excipient and a compound of Formula (IIIi-4), or a pharmaceutically acceptable salt or solvate thereof. In some embodiments is a pharmaceutical composition comprising a pharmaceutically acceptable excipient and a compound of Formula (IVa-1), or a pharmaceutically acceptable salt or solvate thereof. In some embodiments is a pharmaceutical composition comprising a pharmaceutically acceptable excipient and a compound of Formula (IVb-1), or a pharmaceutically acceptable salt or solvate thereof. In some embodiments is a pharmaceutical composition comprising a pharmaceutically acceptable excipient and a compound of Formula (IVc-1), or a pharmaceutically acceptable salt or solvate thereof. In some embodiments is a pharmaceutical composition comprising a pharmaceutically acceptable excipient and a compound of Formula (IVa-2), or a pharmaceutically acceptable salt or solvate thereof. In some embodiments is a pharmaceutical composition comprising a pharmaceutically acceptable excipient and a compound of Formula (IVb-2), or a pharmaceutically acceptable salt or solvate thereof. In some embodiments is a pharmaceutical composition comprising a pharmaceutically acceptable excipient and a compound of Formula (IVc-2), or a pharmaceutically acceptable salt or solvate thereof. In some embodiments is a pharmaceutical composition comprising a pharmaceutically acceptable excipient and a compound of Formula (Va-1), or a pharmaceutically acceptable salt or solvate thereof. In some embodiments is a pharmaceutical composition comprising a pharmaceutically acceptable excipient and a compound of Formula (Vb-1), or a pharmaceutically acceptable salt or solvate thereof. In some embodiments is a pharmaceutical composition comprising a pharmaceutically acceptable excipient and a compound of Formula (Vc-1), or a pharmaceutically acceptable salt or solvate thereof. In some embodiments is a pharmaceutical composition comprising a pharmaceutically acceptable excipient and a compound of Formula (Va-2), or a pharmaceutically acceptable salt or solvate thereof. In some embodiments is a pharmaceutical composition comprising a pharmaceutically acceptable excipient and a compound of Formula (Vb-2), or a pharmaceutically acceptable salt or solvate thereof. In some embodiments is a pharmaceutical composition comprising a pharmaceutically acceptable excipient and a compound of Formula (Vc-2), or a pharmaceutically acceptable salt or solvate thereof. In some embodiments is a pharmaceutical composition comprising a pharmaceutically acceptable excipient and a compound of Formula (XI-1), or a pharmaceutically acceptable salt or solvate thereof. In some embodiments is a pharmaceutical composition comprising a pharmaceutically acceptable excipient and a compound of Formula (XI-2), or a pharmaceutically acceptable salt or solvate thereof. In some embodiments is a pharmaceutical composition comprising a pharmaceutically acceptable excipient and a compound of Formula (XIa), or a pharmaceutically acceptable salt or solvate thereof. In some embodiments is a pharmaceutical composition comprising a pharmaceutically acceptable excipient and a compound of Formula (XIb), or a pharmaceutically acceptable salt or solvate thereof. In some embodiments is a pharmaceutical composition comprising a pharmaceutically acceptable excipient and a compound of Formula (XIc), or a pharmaceutically acceptable salt or solvate thereof. In some embodiments is a pharmaceutical composition comprising a pharmaceutically acceptable excipient and a compound of Formula (XId), or a pharmaceutically acceptable salt or solvate thereof. In some embodiments is a pharmaceutical composition comprising a pharmaceutically acceptable excipient and a compound of Formula (XIe), or a pharmaceutically acceptable salt or solvate thereof. In some embodiments is a pharmaceutical composition comprising a pharmaceutically acceptable excipient and a compound of Formula (XIf), or a pharmaceutically acceptable salt or solvate thereof. In some embodiments is a pharmaceutical composition comprising a pharmaceutically acceptable excipient and a compound of Formula (XII-1), or a pharmaceutically acceptable salt or solvate thereof. In some embodiments is a pharmaceutical composition comprising a pharmaceutically acceptable excipient and a compound of Formula (XII-2), or a pharmaceutically acceptable salt or solvate thereof. In some embodiments is a pharmaceutical composition comprising a pharmaceutically acceptable excipient and a compound of Formula (XIIa), or a pharmaceutically acceptable salt or solvate thereof. In some embodiments is a pharmaceutical composition comprising a pharmaceutically acceptable excipient and a compound of Formula (XIIb), or a pharmaceutically acceptable salt or solvate thereof. In some embodiments is a pharmaceutical composition comprising a pharmaceutically acceptable excipient and a compound of Formula (XIIc), or a pharmaceutically acceptable salt or solvate thereof. In some embodiments is a pharmaceutical composition comprising a pharmaceutically acceptable excipient and a compound of Formula (XIId), or a pharmaceutically acceptable salt or solvate thereof. In some embodiments is a pharmaceutical composition comprising a pharmaceutically acceptable excipient and a compound of Formula (XXI-1), or a pharmaceutically acceptable salt or solvate thereof. In some embodiments is a pharmaceutical composition comprising a pharmaceutically acceptable excipient and a compound of Formula (XXI-2), or a pharmaceutically acceptable salt or solvate thereof. In some embodiments is a pharmaceutical composition comprising a pharmaceutically acceptable excipient and a compound of Formula (XXIa), or a pharmaceutically acceptable salt or solvate thereof. In some embodiments is a pharmaceutical composition comprising a pharmaceutically acceptable excipient and a compound of Formula (XXIb), or a pharmaceutically acceptable salt or solvate thereof. In some embodiments is a pharmaceutical composition comprising a pharmaceutically acceptable excipient and a compound of Formula (XXIc), or a pharmaceutically acceptable salt or solvate thereof. In some embodiments is a pharmaceutical composition comprising a pharmaceutically acceptable excipient and a compound of Formula (XXII-1), or a pharmaceutically acceptable salt or solvate thereof. In some embodiments is a pharmaceutical composition comprising a pharmaceutically acceptable excipient and a compound of Formula (XXII-2), or a pharmaceutically acceptable salt or solvate thereof. In some embodiments is a pharmaceutical composition comprising a pharmaceutically acceptable excipient and a compound of Formula (XXIIa-1), or a pharmaceutically acceptable salt or solvate thereof. In some embodiments is a pharmaceutical composition comprising a pharmaceutically acceptable excipient and a compound of Formula (XXIIa-2), or a pharmaceutically acceptable salt or solvate thereof. In some embodiments is a pharmaceutical composition comprising a pharmaceutically acceptable excipient and a compound of Formula (XXIIb-1), or a pharmaceutically acceptable salt or solvate thereof. In some embodiments is a pharmaceutical composition comprising a pharmaceutically acceptable excipient and a compound of Formula (XXIIb-2), or a pharmaceutically acceptable salt or solvate thereof. In some embodiments is a pharmaceutical composition comprising a pharmaceutically acceptable excipient and a compound of Formula (XXIIc), or a pharmaceutically acceptable salt or solvate thereof. In some embodiments is a pharmaceutical composition comprising a pharmaceutically acceptable excipient and a compound of Formula (XXIId), or a pharmaceutically acceptable salt or solvate thereof. In some embodiments is a pharmaceutical composition comprising a pharmaceutically acceptable excipient and a compound of Formula (XXIIe), or a pharmaceutically acceptable salt or solvate thereof. In some embodiments is a pharmaceutical composition comprising a pharmaceutically acceptable excipient and a compound of Formula (XXIIf), or a pharmaceutically acceptable salt or solvate thereof. In some embodiments is a pharmaceutical composition comprising a pharmaceutically acceptable excipient and a compound of Formula (XXIIg), or a pharmaceutically acceptable salt or solvate thereof. In some embodiments is a pharmaceutical composition comprising a pharmaceutically acceptable excipient and a compound of Formula (XXIII-1), or a pharmaceutically acceptable salt or solvate thereof. In some embodiments is a pharmaceutical composition comprising a pharmaceutically acceptable excipient and a compound of Formula (XXIII-2), or a pharmaceutically acceptable salt or solvate thereof. In some embodiments is a pharmaceutical composition comprising a pharmaceutically acceptable excipient and a compound of Formula (XXIV-1), or a pharmaceutically acceptable salt or solvate thereof. In some embodiments is a pharmaceutical composition comprising a pharmaceutically acceptable excipient and a compound of Formula (XXIV-2), or a pharmaceutically acceptable salt or solvate thereof. Also provided are pharmaceutical compositions comprising one or more compounds disclosed in the “ADDITIONAL EMBODIMENTS”.

In some embodiments of the methods described herein, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments of the methods described herein, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### ADDITIONAL EMBODIMENTS

Embodiment 1. A compound of Formula (I), or a pharmaceutically acceptable salt or solvate thereof:

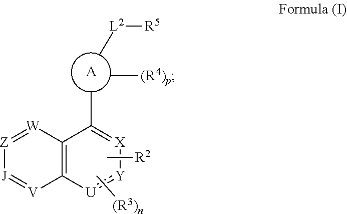

wherein

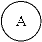

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C_{6 10}aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}, or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, Co-10aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, Co-10aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment 2. The compound of embodiment 1, or a pharmaceutically acceptable salt or solvate thereof, having the structure of Formula (I′):

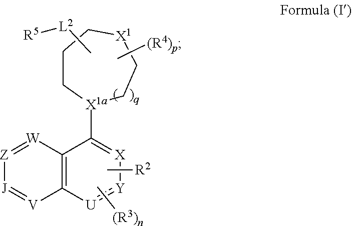

wherein X¹ is selected from C, N, O, S, S(O), and S(O)₂; X^{1a} is selected from N and C(H); and q is 1 or 2.

Embodiment 3. The compound of embodiment 1 or embodiment 2, or a pharmaceutically acceptable salt or solvate thereof, having the structure of Formula (Ia):

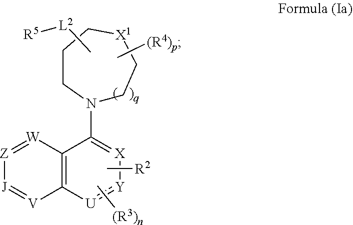

wherein X¹ is selected from C, N, O, S, S(O), and S(O)₂; and q is 1 or 2.

Embodiment 4. The compound of embodiment 3, or a pharmaceutically acceptable salt or solvate thereof, having the structure of Formula (Ib):

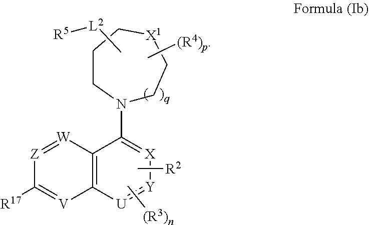

Embodiment 5. The compound of embodiment 4, or a pharmaceutically acceptable salt or solvate thereof, having the structure of Formula (Ic):

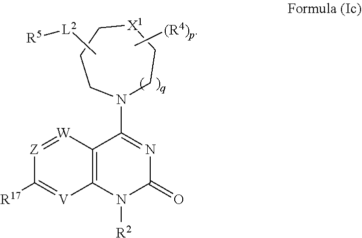

Embodiment 6. The compound of embodiment 4, or a pharmaceutically acceptable salt or solvate thereof, having the structure of Formula (Id):

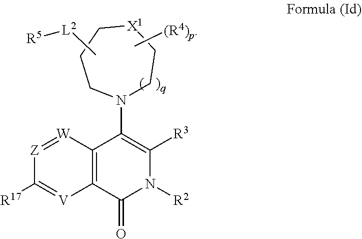

Embodiment 7. The compound of embodiment 4, or a pharmaceutically acceptable salt or solvate thereof, having the structure of Formula (Ie):

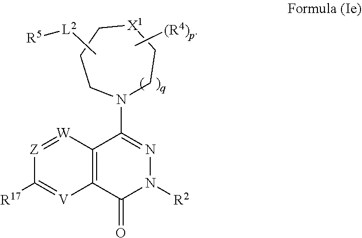

Embodiment 8. The compound of embodiment 4, or a pharmaceutically acceptable salt or solvate thereof, having the structure of Formula (If):

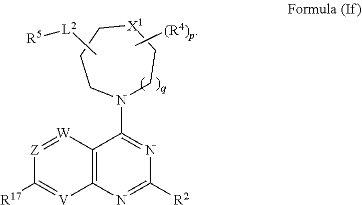

Embodiment 9. The compound of embodiment 4, or a pharmaceutically acceptable salt or solvate thereof, having the structure of Formula (Ig):

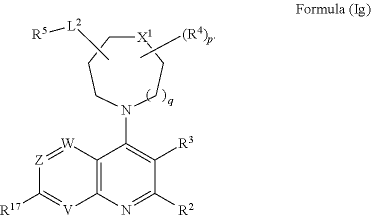

Embodiment 10. A compound of Formula (I″), or a pharmaceutically acceptable salt or solvate thereof:

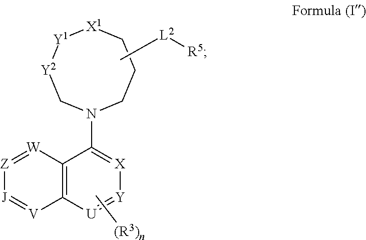

wherein:
X is C or N;
X¹ is selected from C(R⁴)(R⁶), N(R¹²), N(R⁶), O, S, S(O), and S(O);
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁴);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(—NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

Embodiment 11. The compound of embodiment 10, or a pharmaceutically acceptable salt or solvate thereof, wherein Y² is a bond.

Embodiment 12. The compound of embodiment 10, or a pharmaceutically acceptable salt or solvate thereof, wherein Y² is CH₂.

Embodiment 13. The compound of any one of embodiments 10-12, or a pharmaceutically acceptable salt or solvate thereof, wherein Y¹ is CH₂.

Embodiment 14. The compound of any one of embodiments 2-13, or a pharmaceutically acceptable salt or solvate thereof, wherein X¹ is N(H).

Embodiment 15. The compound of any one of embodiments 2-13, or a pharmaceutically acceptable salt or solvate thereof, wherein X¹ is N(R⁴).

Embodiment 16. The compound of any one of embodiments 2-13, or a pharmaceutically acceptable salt or solvate thereof, wherein X¹ is N(-L²-R⁵).

Embodiment 17. The compound of any one of embodiments 2-13, or a pharmaceutically acceptable salt or solvate thereof, wherein X¹ is O.

Embodiment 18. The compound of any one of embodiments 2-13, or a pharmaceutically acceptable salt or solvate thereof, wherein X¹ is S.

Embodiment 19. The compound of any one of embodiments 2-13, or a pharmaceutically acceptable salt or solvate thereof, wherein X¹ is S(O)₂.

Embodiment 20. The compound of any one of embodiments 2-13, or a pharmaceutically acceptable salt or solvate thereof, wherein X¹ is CH₂.

Embodiment 21. The compound of any one of embodiments 2-13, or a pharmaceutically acceptable salt or solvate thereof, wherein X¹ is C(H)(R⁴).

Embodiment 22. The compound of any one of embodiments 2-13, or a pharmaceutically acceptable salt or solvate thereof, wherein X¹ is C(R⁴)₂.

Embodiment 23. The compound of any one of embodiments 2-13, or a pharmaceutically acceptable salt or solvate thereof, wherein X¹ is C(H)(-L²-R⁵).

Embodiment 24. The compound of any one of embodiments 2-23, or a pharmaceutically acceptable salt or solvate thereof, wherein q is 1.

Embodiment 25. The compound of any one of embodiments 2-23, or a pharmaceutically acceptable salt or solvate thereof, wherein q is 2.

Embodiment 26. The compound of any one of embodiments 1-25, or a pharmaceutically acceptable salt or solvate thereof, wherein V is C(R¹⁶).

Embodiment 27. The compound of any one of embodiments 1-26, or a pharmaceutically acceptable salt or solvate thereof, wherein V is C(H).

Embodiment 28. The compound of any one of embodiments 1-25, or a pharmaceutically acceptable salt or solvate thereof, wherein V is N.

Embodiment 29. The compound of any one of embodiments 1-28, or a pharmaceutically acceptable salt or solvate thereof, wherein W is C(R¹⁸).

Embodiment 30. The compound of embodiment 29, or a pharmaceutically acceptable salt or solvate thereof, wherein W is C(H).

Embodiment 31. The compound of any one of embodiments 1-28, or a pharmaceutically acceptable salt or solvate thereof, wherein W is N.

Embodiment 32. The compound of any one of embodiments 1-31, or a pharmaceutically acceptable salt or solvate thereof, wherein Z is C(R⁸).

Embodiment 33. The compound of embodiment 32, or a pharmaceutically acceptable salt or solvate thereof, wherein Z is C(H).

Embodiment 34. The compound of any one of embodiments 1-31, or a pharmaceutically acceptable salt or solvate thereof, wherein Z is N.

Embodiment 35. A compound of Formula (II), or a pharmaceutically acceptable salt or solvate thereof:

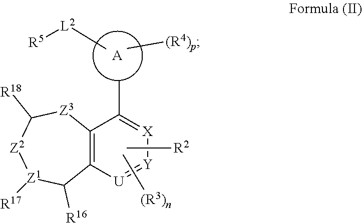

wherein:

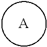

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸); Z¹ is N or C(R⁶);
Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};

R⁵ is hydrogen, or a group other than an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, Co-10aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};

R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

Embodiment 36. The compound of embodiment 35, or a pharmaceutically acceptable salt or solvate thereof, having the structure of Formula (IIa):

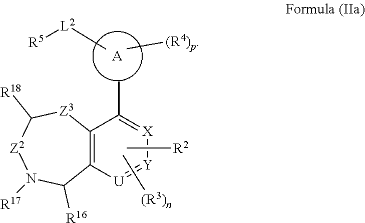

Embodiment 37. The compound of embodiment 35, or a pharmaceutically acceptable salt or solvate thereof, having the structure of Formula (IIb):

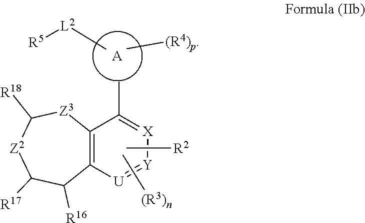

Embodiment 38. The compound of embodiment 35 or embodiment 36, or a pharmaceutically acceptable salt or solvate thereof, having the structure of Formula (IIc′):

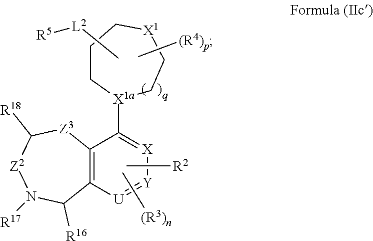

wherein X¹ is selected from C, N, O, S, S(O), and S(O)₂; X^{1a} is selected from N and C(H); and q is 1 or 2.

Embodiment 39. The compound of embodiment 38, or a pharmaceutically acceptable salt or solvate thereof, having the structure of Formula (IIc):

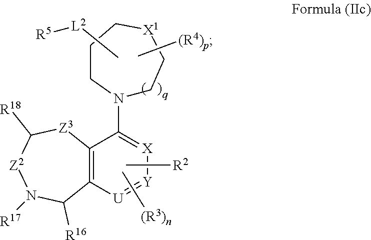

wherein X¹ is selected from C, N, O, S, S(O), and S(O)₂; and q is 1 or 2.

Embodiment 40. The compound of embodiment 35 or embodiment 37, or a pharmaceutically acceptable salt or solvate thereof, having the structure of Formula (IId′):

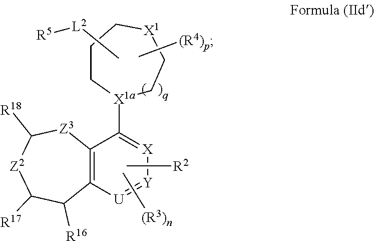

wherein X¹ is selected from C, N, O, S, S(O), and S(O)₂; X^{1a} is selected from N and C(H); and q is 1 or 2.

Embodiment 41. The compound of embodiment 40, or a pharmaceutically acceptable salt or solvate thereof, having the structure of Formula (IId):

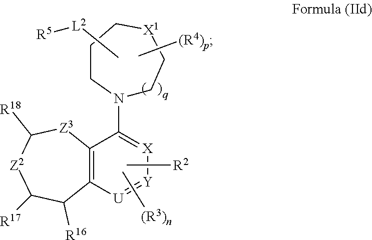

wherein X¹ is selected from C, N, O, S, S(O), and S(O)₂; and q is 1 or 2.

Embodiment 42. The compound of any one of embodiments 38-41, or a pharmaceutically acceptable salt or solvate thereof, wherein X¹ is N(H).

Embodiment 43. The compound of any one of embodiments 38-41, or a pharmaceutically acceptable salt or solvate thereof, wherein X¹ is N(R⁴).

Embodiment 44. The compound of any one of embodiments 38-41, or a pharmaceutically acceptable salt or solvate thereof, wherein X¹ is N(-L²-R⁵).

Embodiment 45. The compound of any one of embodiments 38-41, or a pharmaceutically acceptable salt or solvate thereof, wherein X¹ is O.

Embodiment 46. The compound of any one of embodiments 38-41, or a pharmaceutically acceptable salt or solvate thereof, wherein X¹ is S.

Embodiment 47. The compound of any one of embodiments 38-41, or a pharmaceutically acceptable salt or solvate thereof, wherein X¹ is S(O)₂.

Embodiment 48. The compound of any one of embodiments 38-41, or a pharmaceutically acceptable salt or solvate thereof, wherein X¹ is CH₂.

Embodiment 49. The compound of any one of embodiments 38-41, or a pharmaceutically acceptable salt or solvate thereof, wherein X¹ is C(H)(R⁴).

Embodiment 50. The compound of any one of embodiments 38-41, or a pharmaceutically acceptable salt or solvate thereof, wherein X¹ is C(R⁴)₂.

Embodiment 51. The compound of any one of embodiments 38-41, or a pharmaceutically acceptable salt or solvate thereof, wherein X¹ is C(H)(-L²-R⁵).

Embodiment 52. The compound of any one of embodiments of any one of embodiments 38-51, or a pharmaceutically acceptable salt or solvate thereof, wherein q is 1.

Embodiment 53. The compound of any one of embodiments 38-51, or a pharmaceutically acceptable salt or solvate thereof, wherein q is 2.

Embodiment 54. The compound of any one of embodiments 35-53, or a pharmaceutically acceptable salt or solvate thereof, wherein Z³ is absent.

Embodiment 55. The compound of any one of embodiments 35-53, or a pharmaceutically acceptable salt or solvate thereof, wherein Z² is C(R⁸)(R⁹).

Embodiment 56. The compound of any one of embodiments 35-55, or a pharmaceutically acceptable salt or solvate thereof, wherein R⁹ is hydrogen.

Embodiment 57. The compound of any one of embodiments 35-55, or a pharmaceutically acceptable salt or solvate thereof, wherein X is C.

Embodiment 58. The compound of any one of embodiments 35-55, or a pharmaceutically acceptable salt or solvate thereof, wherein X is N.

Embodiment 59. The compound of any one of embodiments 35-58, or a pharmaceutically acceptable salt or solvate thereof, wherein Y is C.

Embodiment 60. The compound of any one of embodiments 35-58, or a pharmaceutically acceptable salt or solvate thereof, wherein Y is N.

Embodiment 61. The compound of any one of embodiments 35-58, or a pharmaceutically acceptable salt or solvate thereof, wherein Y is C(O).

Embodiment 62. The compound of any one of embodiments 35-61, or a pharmaceutically acceptable salt or solvate thereof, wherein U is C.

Embodiment 63. The compound of any one of embodiments 35-61, or a pharmaceutically acceptable salt or solvate thereof, wherein U is N.

Embodiment 64. The compound of any one of embodiments 35-60, or a pharmaceutically acceptable salt or solvate thereof, wherein U is C(O).

Embodiment 65. The compound of any one of embodiments 42-53, or a pharmaceutically acceptable salt or solvate thereof, having the structure of Formula (IIe):

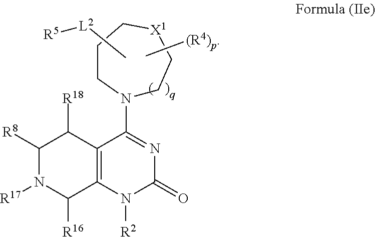

Embodiment 66. The compound of any one of embodiments 42-53, or a pharmaceutically acceptable salt or solvate thereof, having the structure of Formula (IIf):

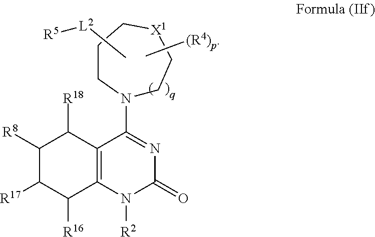

Embodiment 67. The compound of any one of embodiments 42-53, or a pharmaceutically acceptable salt or solvate thereof, having the structure of Formula (IIg):

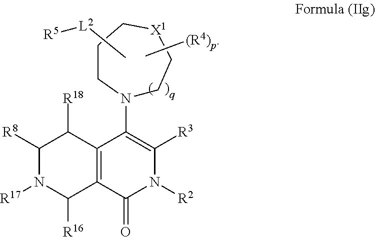

Embodiment 68. The compound of any one of embodiments 42-53, or a pharmaceutically acceptable salt or solvate thereof, having the structure of Formula (IIh):

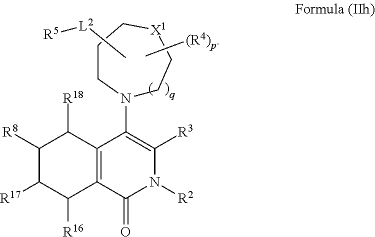

Embodiment 69. The compound of any one of embodiments 42-53, or a pharmaceutically acceptable salt or solvate thereof, having the structure of Formula (III):

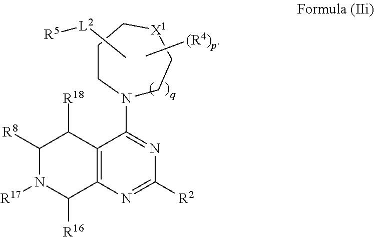

Embodiment 70. The compound of any one of embodiments 42-53, or a pharmaceutically acceptable salt or solvate thereof, having the structure of Formula (IIj):

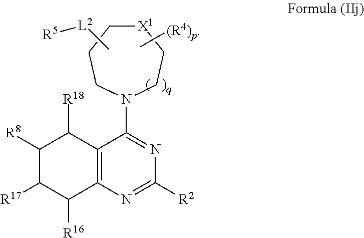

Embodiment 71. The compound of any one of embodiments 42-53, or a pharmaceutically acceptable salt or solvate thereof, having the structure of Formula (IIk):

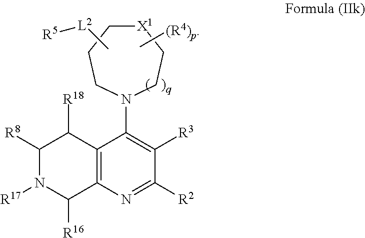

Embodiment 72. The compound of any one of embodiments 42-53, or a pharmaceutically acceptable salt or solvate thereof, having the structure of Formula (IIm):

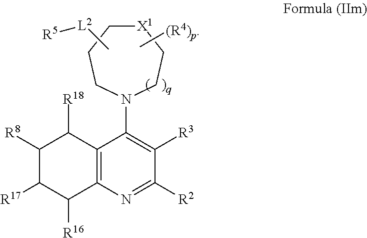

Embodiment 73. A compound of Formula (IIIa)-(IIIi), or a pharmaceutically acceptable salt or solvate thereof:

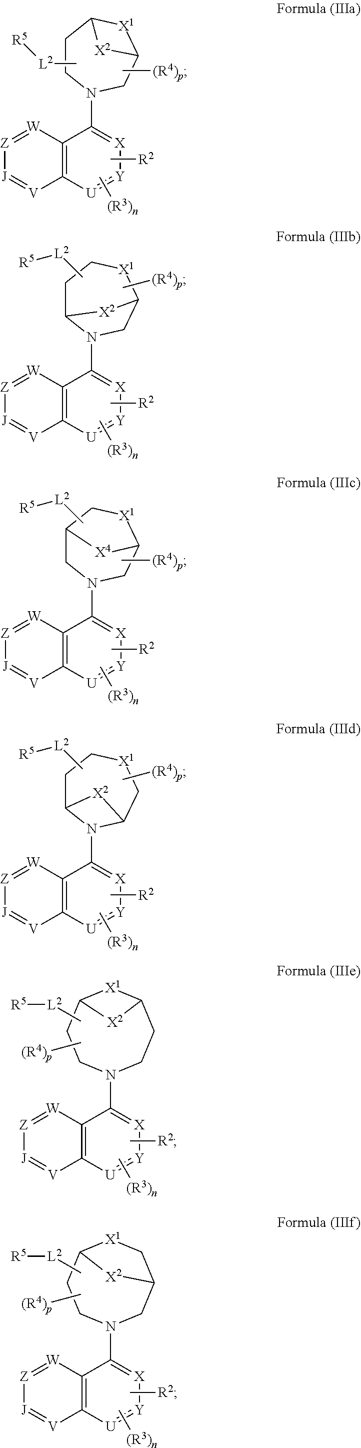

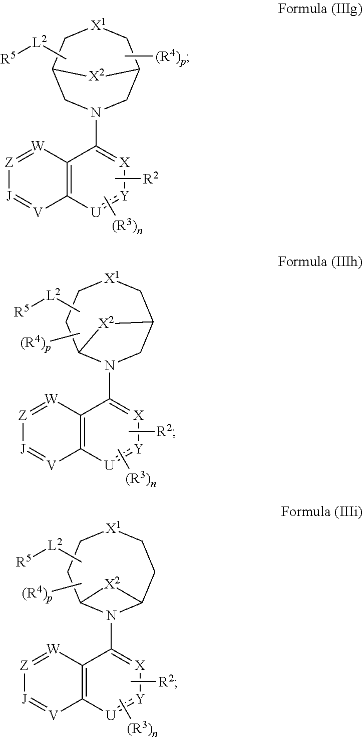

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X⁵, —CH₂—, —X⁵CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X⁵—, —C(R⁴)₂—, —C(R⁴)₂X⁵—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment 74. The compound of embodiment 73, or a pharmaceutically acceptable salt or solvate thereof, having the structure of Formula (IIIa):

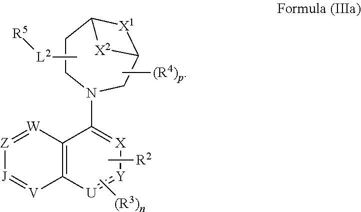

Embodiment 75. The compound of embodiment 73, or a pharmaceutically acceptable salt or solvate thereof, having the structure of Formula (IIIb):

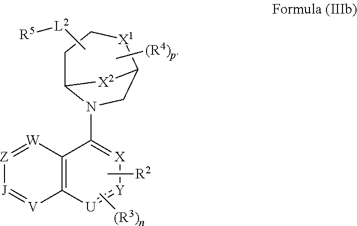

Embodiment 76. The compound of embodiment 73, or a pharmaceutically acceptable salt or solvate thereof, having the structure of Formula (IIId):

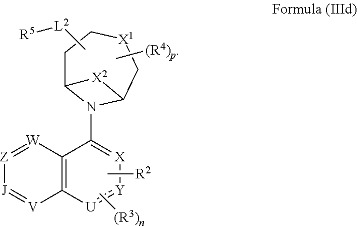

Embodiment 77. The compound of embodiment 73, or a pharmaceutically acceptable salt or solvate thereof, having the structure of Formula (IIIe):

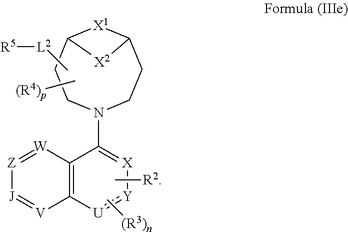

Embodiment 78. The compound of embodiment 73, or a pharmaceutically acceptable salt or solvate thereof, having the structure of Formula (IIIf):

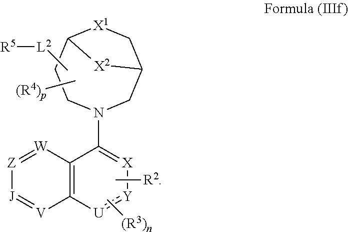

Embodiment 79. The compound of embodiment 73, or a pharmaceutically acceptable salt or solvate thereof, having the structure of Formula (IIIg):

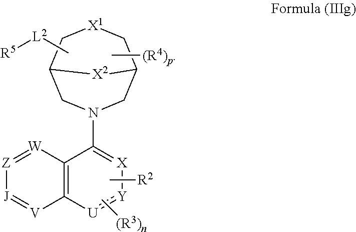

Embodiment 80. The compound of embodiment 73, or a pharmaceutically acceptable salt or solvate thereof, having the structure of Formula (IIIh):

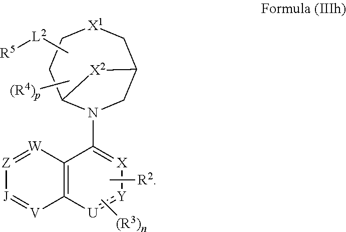

Embodiment 81. The compound of embodiment 73, or a pharmaceutically acceptable salt or solvate thereof, having the structure of Formula (IIIi):

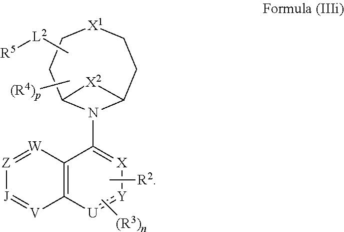

Embodiment 82. The compound of embodiment 73, or a pharmaceutically acceptable salt or solvate thereof, having the structure of Formula (IIIc):

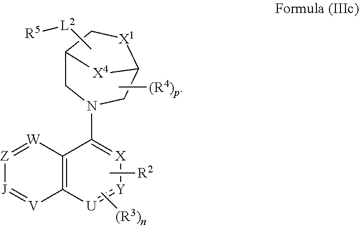

Embodiment 83. The compound of embodiment 82, or a pharmaceutically acceptable salt or solvate thereof, wherein X⁴ is selected from —CH₂— and —CH₂CH₂—.

Embodiment 84. The compound of any one of embodiments 73-81, or a pharmaceutically acceptable salt or solvate thereof, wherein X² is selected from —CH₂— and —CH₂CH₂—.

Embodiment 85. A compound of Formula (IVa), (IVb), or (IVc), or a pharmaceutically acceptable salt or solvate thereof:

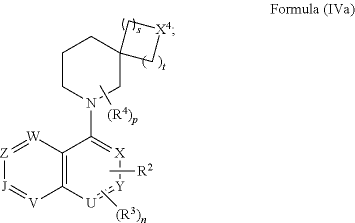

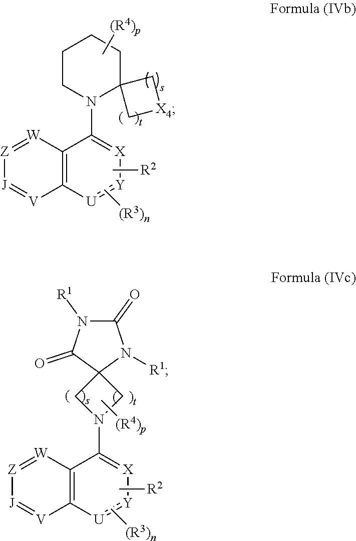

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a}, R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20b}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
tis 1, 2, 3, 4, or 5; wherein s+t≥2; and
[symbol] indicates a single or double bond such that all valences are satisfied.

Embodiment 86. The compound of embodiment 85, or a pharmaceutically acceptable salt or solvate thereof, having the structure of Formula (IVa):

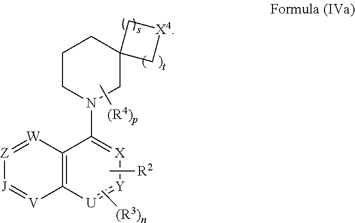

Embodiment 87. The compound of embodiment 85, or a pharmaceutically acceptable salt or solvate thereof, having the structure of Formula (IVb):

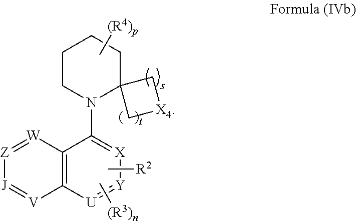

Embodiment 88. The compound of any one of embodiments 85-87, or a pharmaceutically acceptable salt or solvate thereof, wherein s is 1 or 2.

Embodiment 89. The compound of any one of embodiments 85-88, or a pharmaceutically acceptable salt or solvate thereof, wherein t is 1 or 2.

Embodiment 90. The compound of any one of embodiments 85-89, or a pharmaceutically acceptable salt or solvate thereof, wherein X⁴ is N(R¹).

Embodiment 91. The compound of embodiment 85, or a pharmaceutically acceptable salt or solvate thereof, having the structure of Formula (IVc):

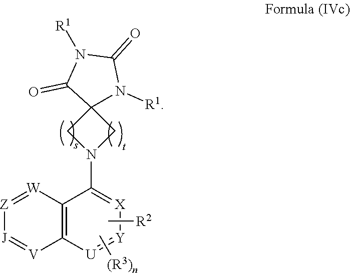

Embodiment 92. A compound of Formula (Va), (Vb), or (Vc), or a pharmaceutically acceptable salt or solvate thereof:

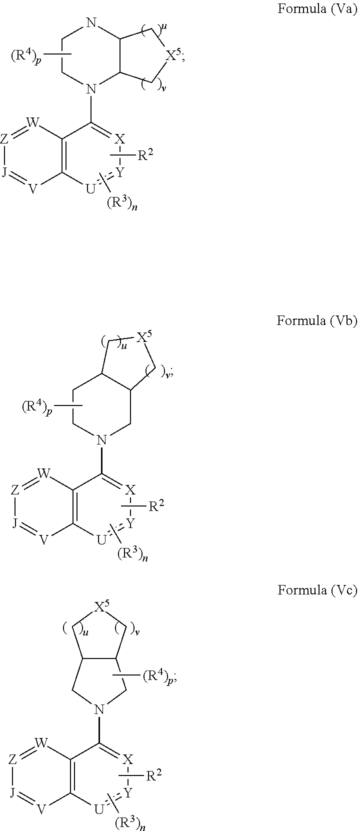

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20b}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

Embodiment 93. The compound of embodiment 92, or a pharmaceutically acceptable salt or solvate thereof, having the structure of Formula (Va):

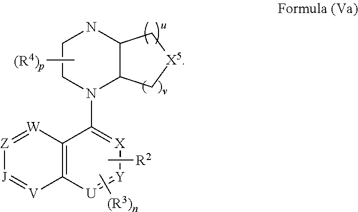

Embodiment 94. The compound of embodiment 92, or a pharmaceutically acceptable salt or solvate thereof, having the structure of Formula (Vb):

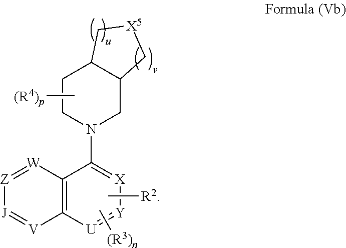

Embodiment 95. The compound of embodiment 92, or a pharmaceutically acceptable salt or solvate thereof, having the structure of Formula (Vc):

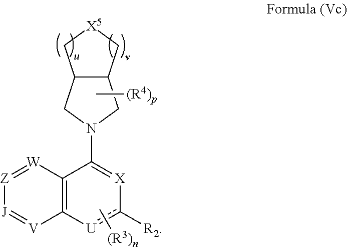

Embodiment 96. The compound of any one of embodiments 92-95, or a pharmaceutically acceptable salt or solvate thereof, wherein u is 0 or 1.

Embodiment 97. The compound of any one of embodiments 92-95, or a pharmaceutically acceptable salt or solvate thereof, wherein v is 0 or 1.

Embodiment 98. The compound of any one of embodiments 92-97, or a pharmaceutically acceptable salt or solvate thereof, wherein X⁵ is N(R¹).

Embodiment 99. The compound of any one of embodiments 85-98, or a pharmaceutically acceptable salt or solvate thereof, wherein R¹ is hydrogen.

Embodiment 100. The compound of any one of embodiments 84-98, or a pharmaceutically acceptable salt or solvate thereof, wherein R¹ is -L²-R⁵.

Embodiment 101. The compound of any one of embodiments 73-100, or a pharmaceutically acceptable salt or solvate thereof, wherein Y is C.

Embodiment 102. The compound of any one of embodiments 73-100, or a pharmaceutically acceptable salt or solvate thereof, wherein Y is N.

Embodiment 103. The compound of any one of embodiments 73-100, or a pharmaceutically acceptable salt or solvate thereof, wherein Y is C(O).

Embodiment 104. The compound of any one of embodiments 73-103, or a pharmaceutically acceptable salt or solvate thereof, wherein X is C.

Embodiment 105. The compound of any one of embodiments 73-103, or a pharmaceutically acceptable salt or solvate thereof, wherein X is N.

Embodiment 106. The compound of any one of embodiments 73-105, or a pharmaceutically acceptable salt or solvate thereof, wherein U is C.

Embodiment 107. The compound of any one of embodiments 73-105, or a pharmaceutically acceptable salt or solvate thereof, wherein U is N.

Embodiment 108. The compound of any one of embodiments 73-105, or a pharmaceutically acceptable salt or solvate thereof, wherein U is C(O).

Embodiment 109. The compound of any one of embodiments 73-108, or a pharmaceutically acceptable salt or solvate thereof, wherein Z is C(R⁸).

Embodiment 110. The compound of any one of embodiments 73-109, or a pharmaceutically acceptable salt or solvate thereof, wherein R⁸ is hydrogen.

Embodiment 111. The compound of any one of embodiments 73-108, or a pharmaceutically acceptable salt or solvate thereof, wherein Z is N.

Embodiment 112. The compound of any one of embodiments 73-111, or a pharmaceutically acceptable salt or solvate thereof, wherein V is C(R¹⁶).

Embodiment 113. The compound of any one of embodiments 73-112, or a pharmaceutically acceptable salt or solvate thereof, wherein V is C(H).

Embodiment 114. The compound of any one of embodiments 73-111, or a pharmaceutically acceptable salt or solvate thereof, wherein V is N.

Embodiment 115. The compound of any one of embodiments 73-114, or a pharmaceutically acceptable salt or solvate thereof, wherein J is C(R¹⁷).

Embodiment 116. The compound of any one of embodiments 73-115, or a pharmaceutically acceptable salt or solvate thereof, wherein W is C(R¹⁸).

Embodiment 117. The compound of embodiment 116, or a pharmaceutically acceptable salt or solvate thereof, wherein W is C(H).

Embodiment 118. The compound of any one of embodiments 73-117, or a pharmaceutically acceptable salt or solvate thereof, wherein W is N.

Embodiment 119. The compound of any one of embodiments 1-118, or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}.

Embodiment 120. The compound of any one of embodiments 1-119, or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}.

Embodiment 121. The compound of any one of embodiments 1-120, or a pharmaceutically acceptable salt or solvate thereof, wherein R² is —OR¹².

Embodiment 122. The compound of any one of embodiments 1-121, or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}.

Embodiment 123. The compound of any one of embodiments 1-121, or a pharmaceutically acceptable salt or solvate thereof, wherein R¹² is C₁₋₆alkyl optionally substituted with one, two, or three R^{20d}.

Embodiment 124. The compound of any one of embodiments 1-121, or a pharmaceutically acceptable salt or solvate thereof, wherein R¹² is C₂₋₉heterocycloalkyl optionally substituted with one, two, or three R^{20d}.

Embodiment 125. The compound of any one of embodiments 1-121, or a pharmaceutically acceptable salt or solvate thereof, wherein R¹² is —CH₂—C₂₋₉heterocycloalkyl optionally substituted with one, two, or three R^{20d}.

Embodiment 126. The compound of any one of embodiments 1-125, or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³).

Embodiment 127. The compound of any one of embodiments 1-126, or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

Embodiment 128. The compound of any one of embodiments 1-119, or a pharmaceutically acceptable salt or solvate thereof, wherein R² is selected from

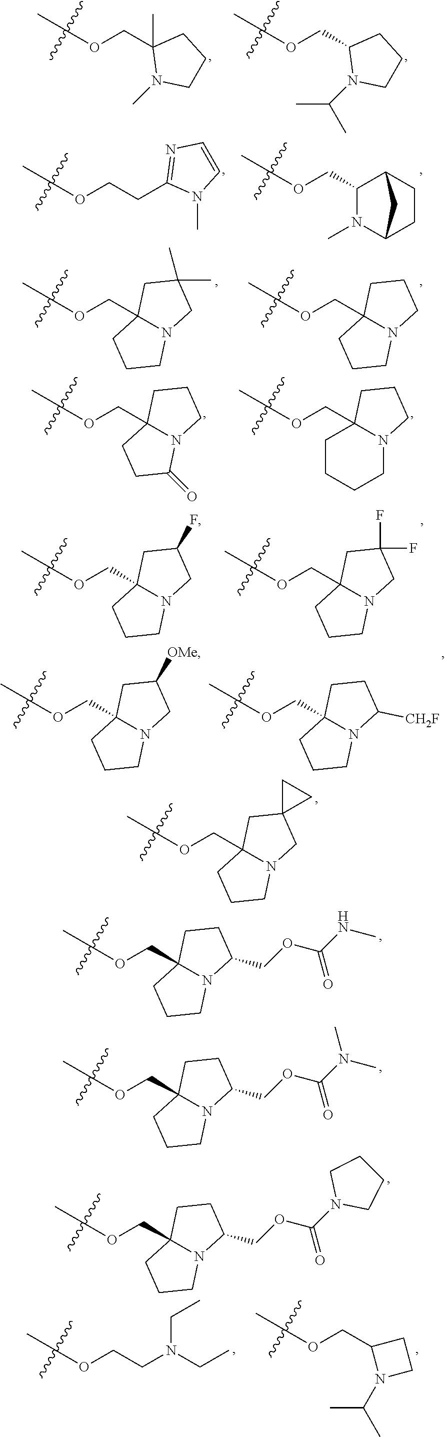

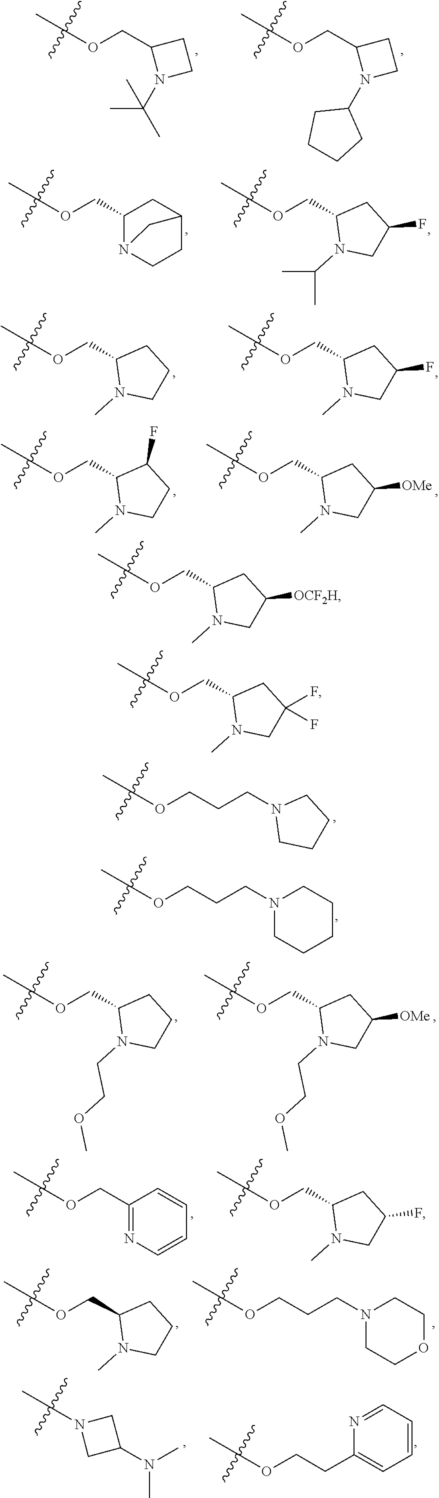

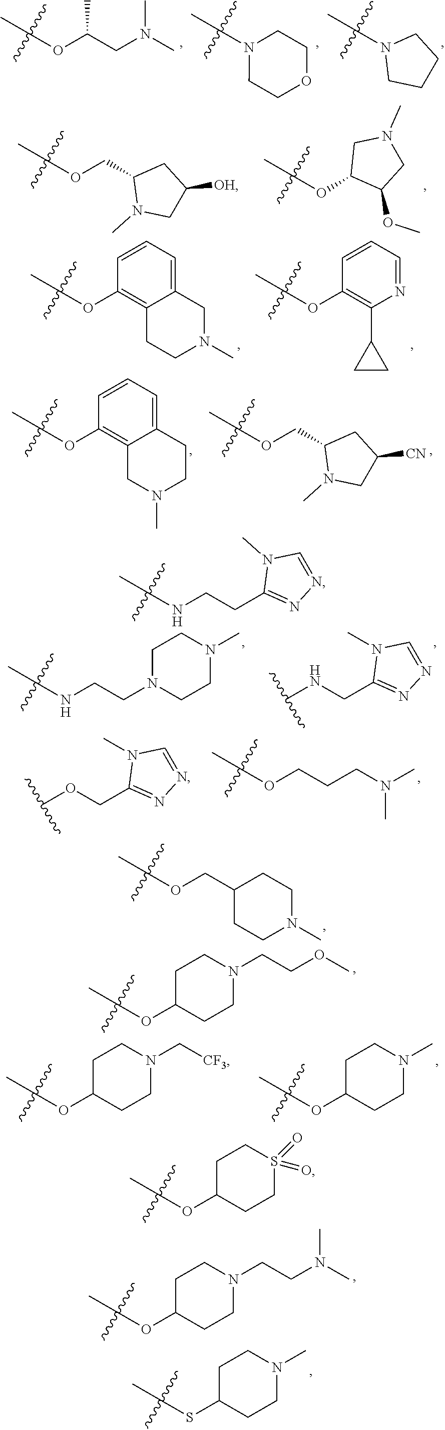

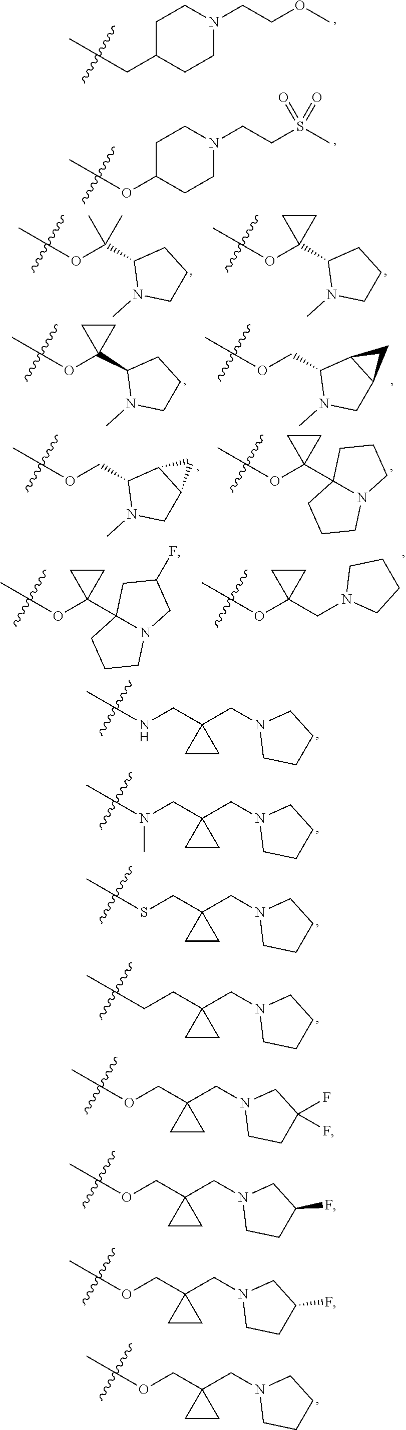

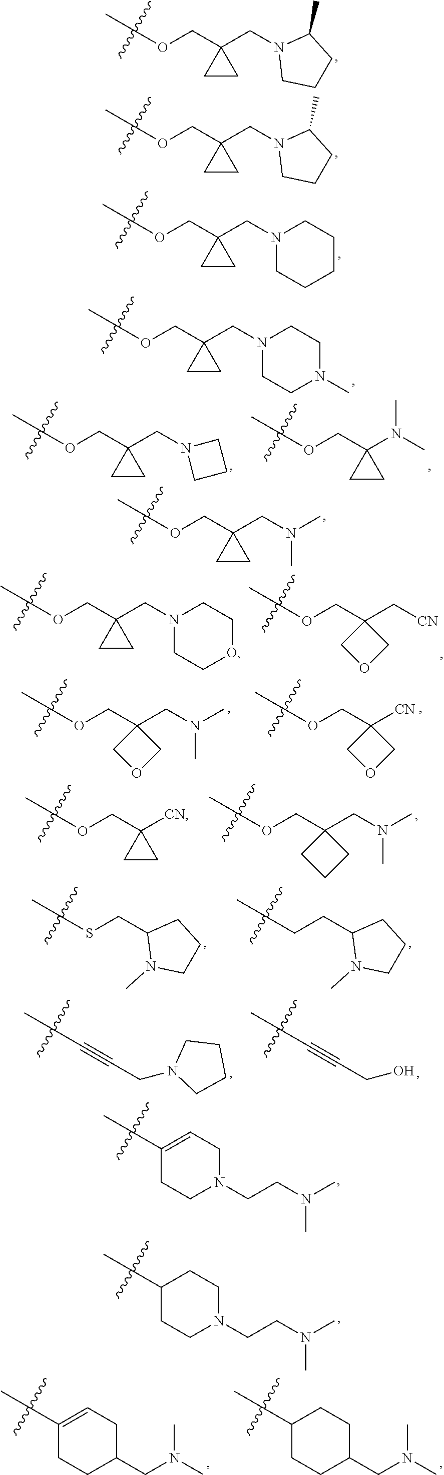

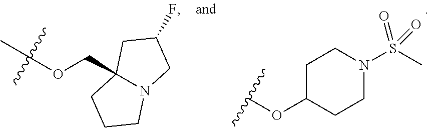 [SMILES: *OC[C@]12CCCN1C[C@@H](F)C2 ; *OC1CCN(S(C)(=O)=O)CC1]

Embodiment 129. The compound of any one of embodiments 1-128, or a pharmaceutically acceptable salt or solvate thereof, wherein L¹ is a bond.

Embodiment 130. The compound of any one of embodiments 1-128, or a pharmaceutically acceptable salt or solvate thereof, wherein L¹ is O.

Embodiment 131. The compound of any one of embodiments 1-130, or a pharmaceutically acceptable salt or solvate thereof, wherein R¹⁹ is C₁₋₉heteroaryl optionally substituted with one, two, or three R²⁰ⁱ.

Embodiment 132. The compound of any one of embodiments 1-130, or a pharmaceutically acceptable salt or solvate thereof, wherein R¹⁹ is C₆₋₁₀aryl optionally substituted with one, two, or three R²⁰ⁱ.

Embodiment 133. The compound of any one of embodiments 1-132, or a pharmaceutically acceptable salt or solvate thereof, wherein L² is selected from a bond, C₁-C₆alkyl, and —C(O)—.

Embodiment 134. The compound of any one of embodiments 1-133, or a pharmaceutically acceptable salt or solvate thereof, wherein L² is a bond.

Embodiment 135. The compound of any one of embodiments 1-133, or a pharmaceutically acceptable salt or solvate thereof, wherein L² is a C₁-C₆alkyl.

Embodiment 136. The compound of any one of embodiments 1-135, or a pharmaceutically acceptable salt or solvate thereof, wherein R⁵ is hydrogen.

Embodiment 137. The compound of any one of embodiments 1-135, or a pharmaceutically acceptable salt or solvate thereof, wherein R⁵ is a group other than an electrophilic moiety capable of forming a covalent bond with the cysteine residue at position 12 of a KRAS protein.

Embodiment 138. The compound of embodiment 137, or a pharmaceutically acceptable salt or solvate thereof, wherein R⁵ is —H, —NH₂, —OH, —NH(C₁₋₆ alkyl),

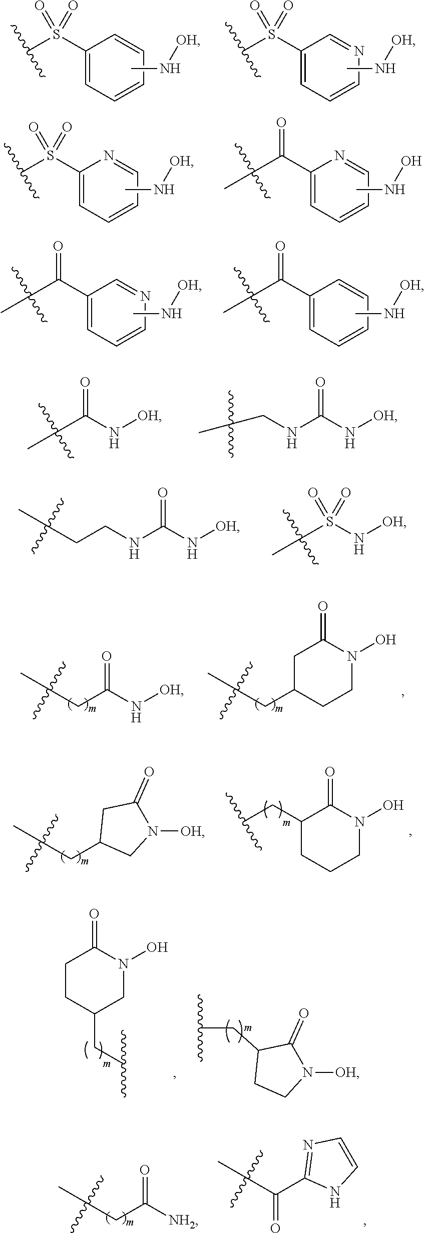

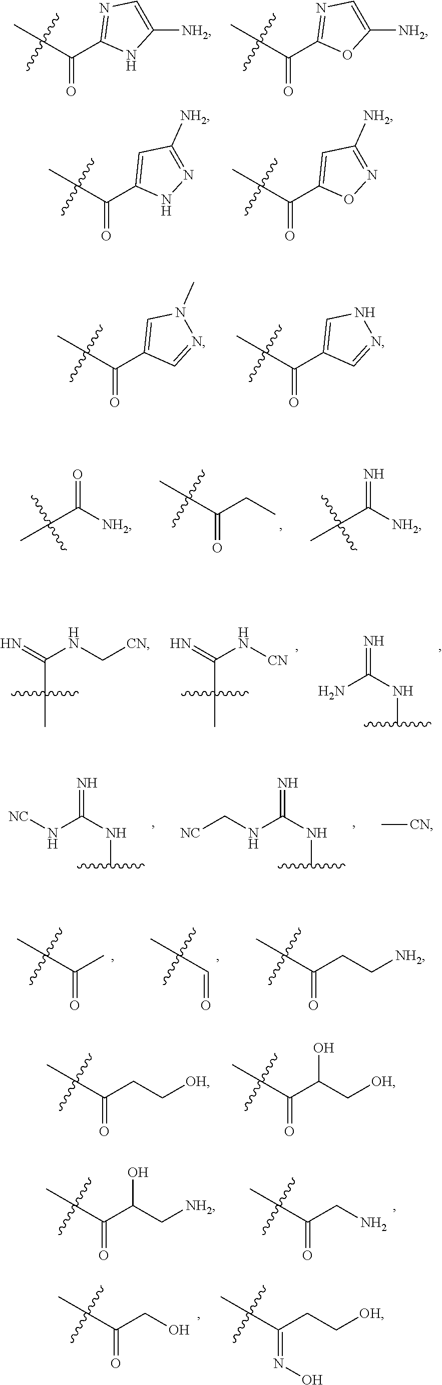

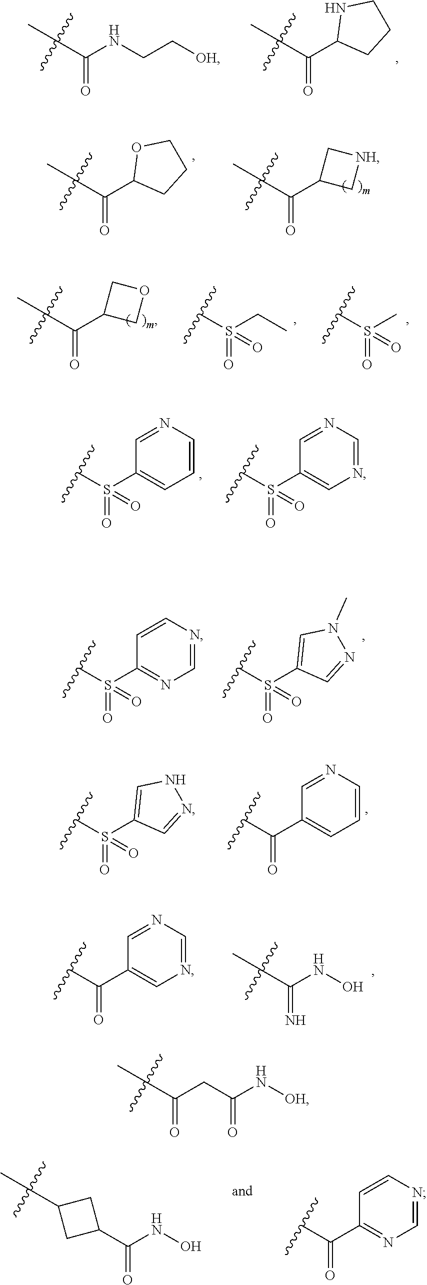

and m, when present, is 0, 1, 2, or 3.

Embodiment 139. A compound selected from:

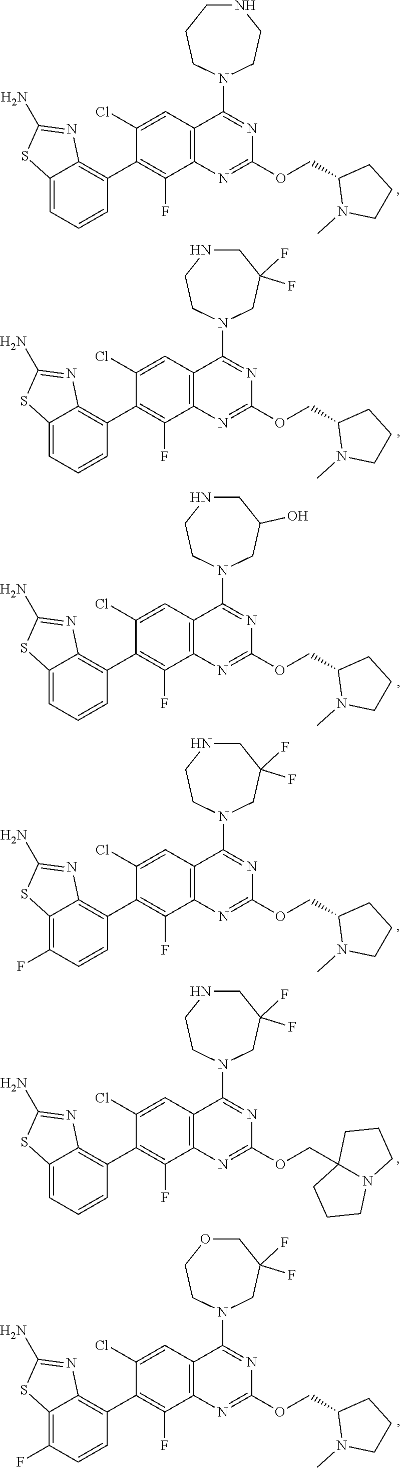

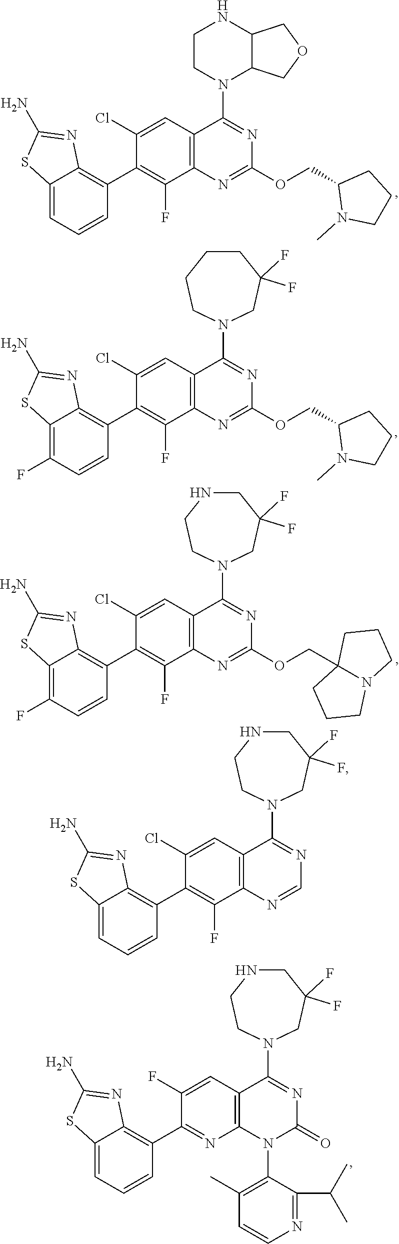

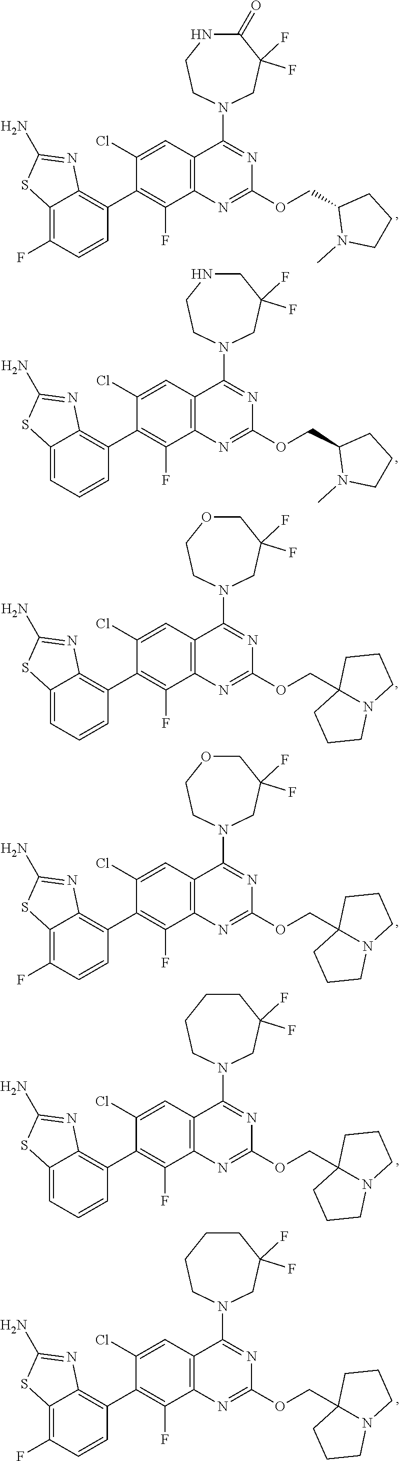

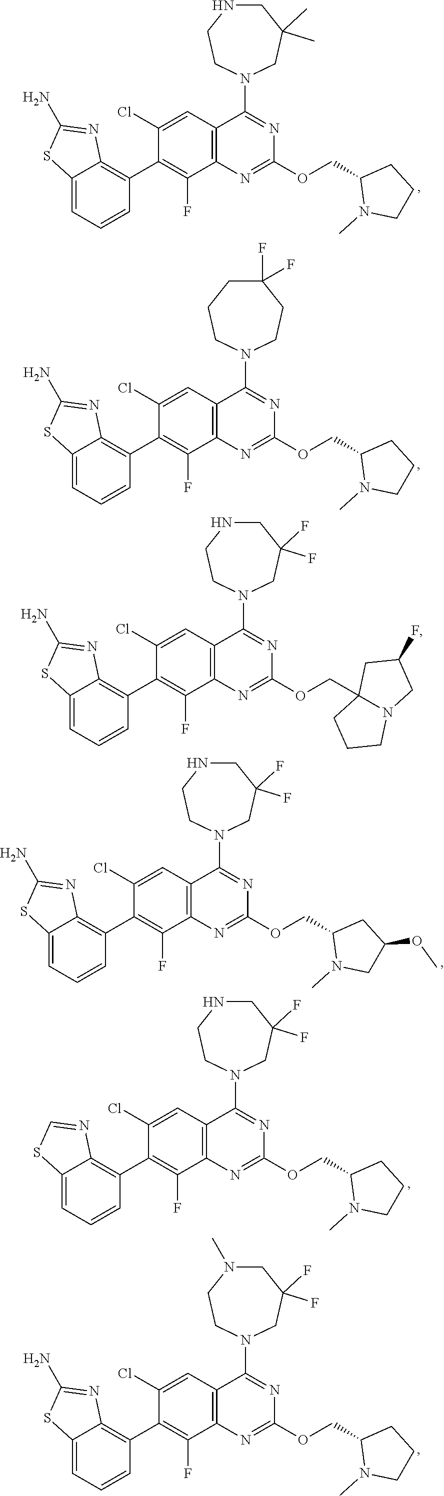

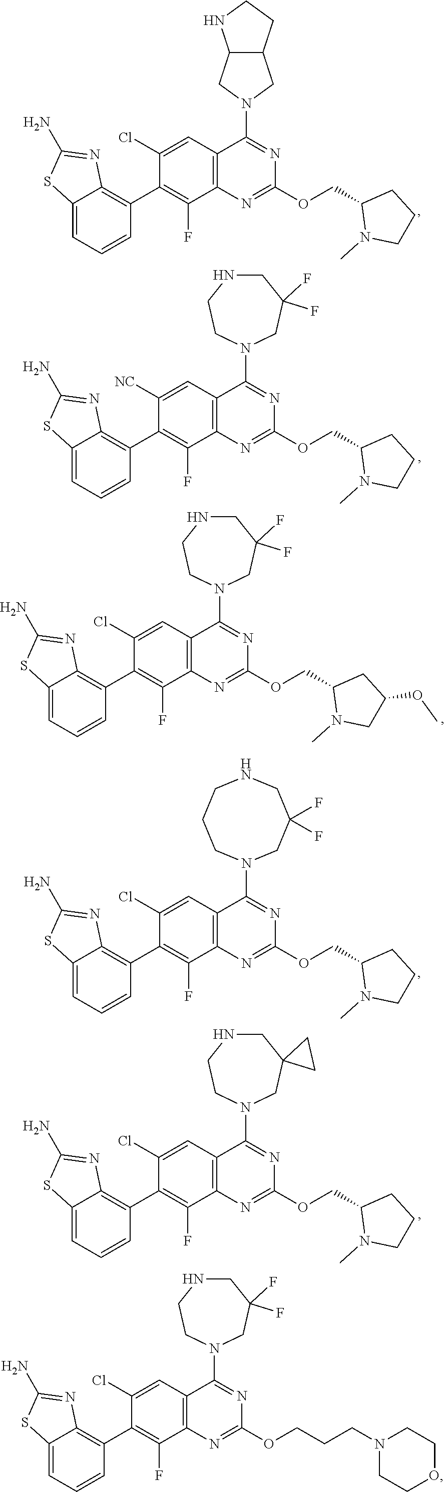

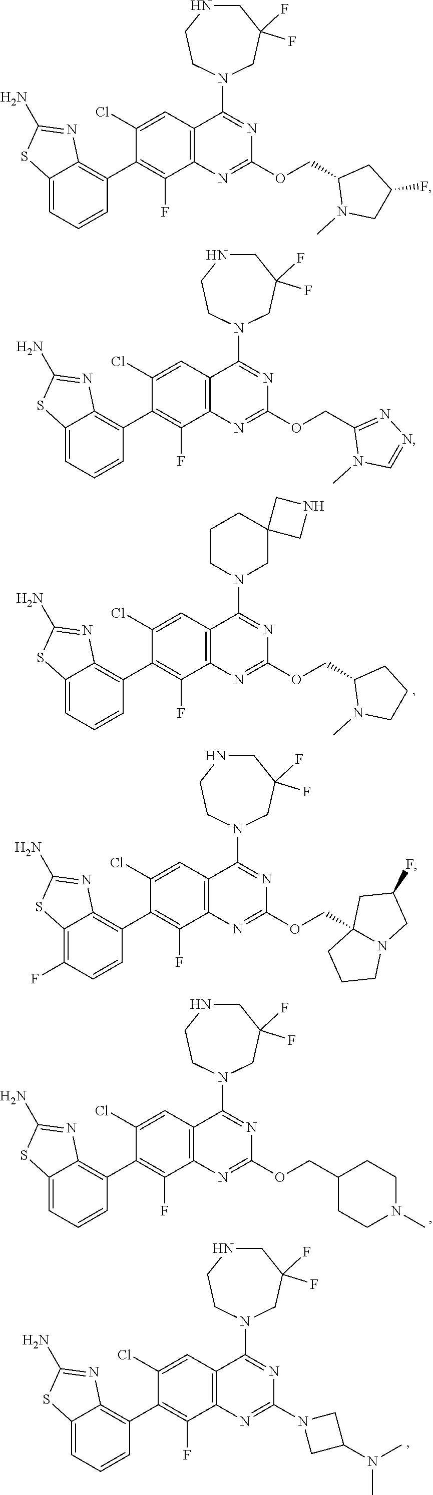

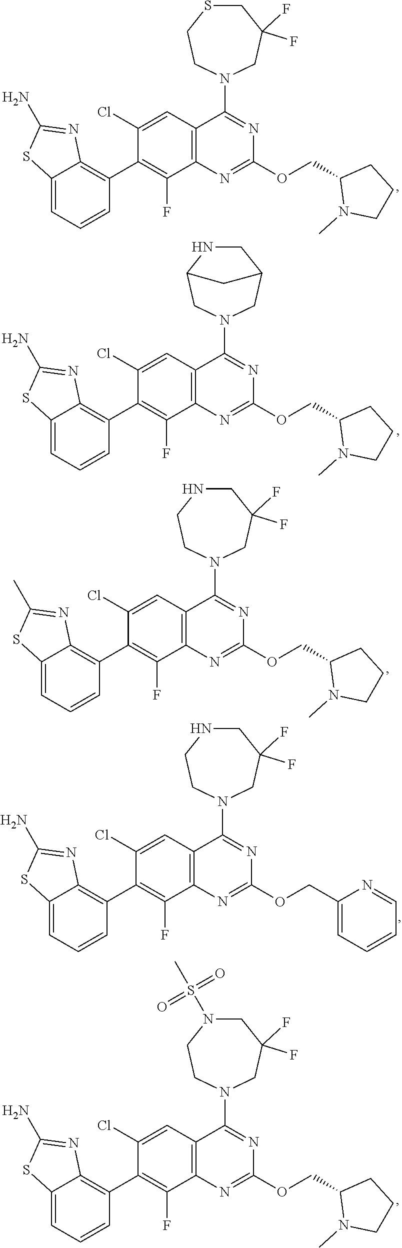

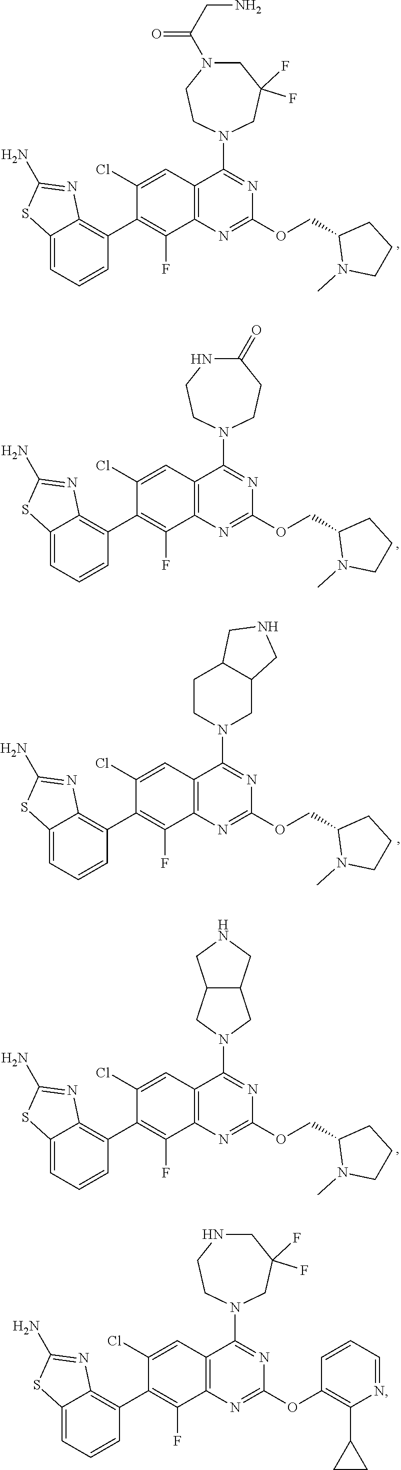 [SMILES: CN1CCC[C@H]1COc1nc(N2CCN(C(=O)CN)CC(F)(F)C2)c2cc(Cl)c(-c3cccc4sc(N)nc34)c(F)c2n1 ; CN1CCC[C@H]1COc1nc(N2CCNC(=O)CC2)c2cc(Cl)c(-c3cccc4sc(N)nc34)c(F)c2n1 ; CN1CCC[C@H]1COc1nc(N2CCC3CNCC3C2)c2cc(Cl)c(-c3cccc4sc(N)nc34)c(F)c2n1 ; CN1CCC[C@H]1COc1nc(N2CC3CNCC3C2)c2cc(Cl)c(-c3cccc4sc(N)nc34)c(F)c2n1 ; Nc1nc2c(-c3c(Cl)cc4c(N5CCNCC(F)(F)C5)nc(Oc5cccnc5C5CC5)nc4c3F)cccc2s1]

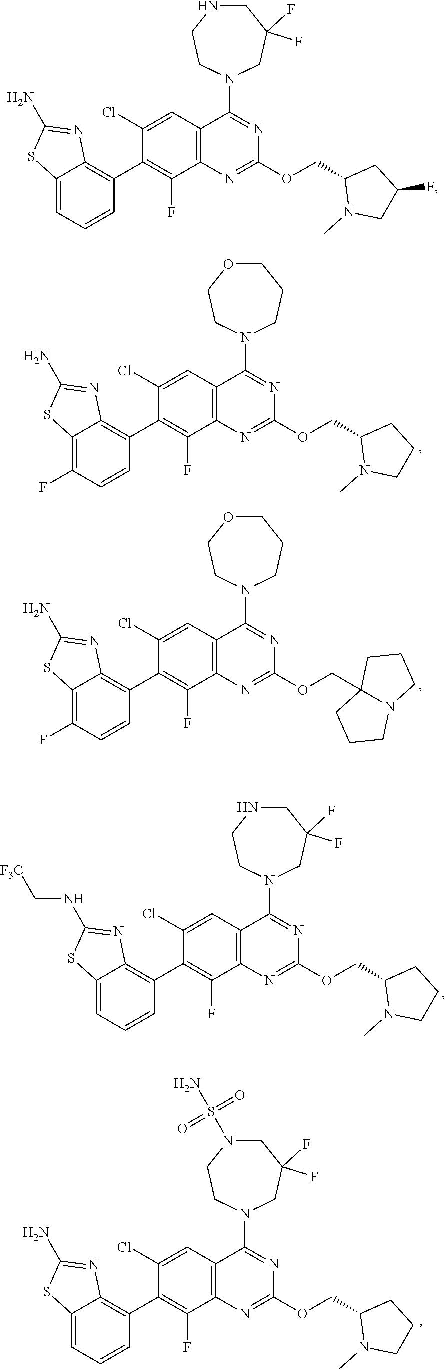

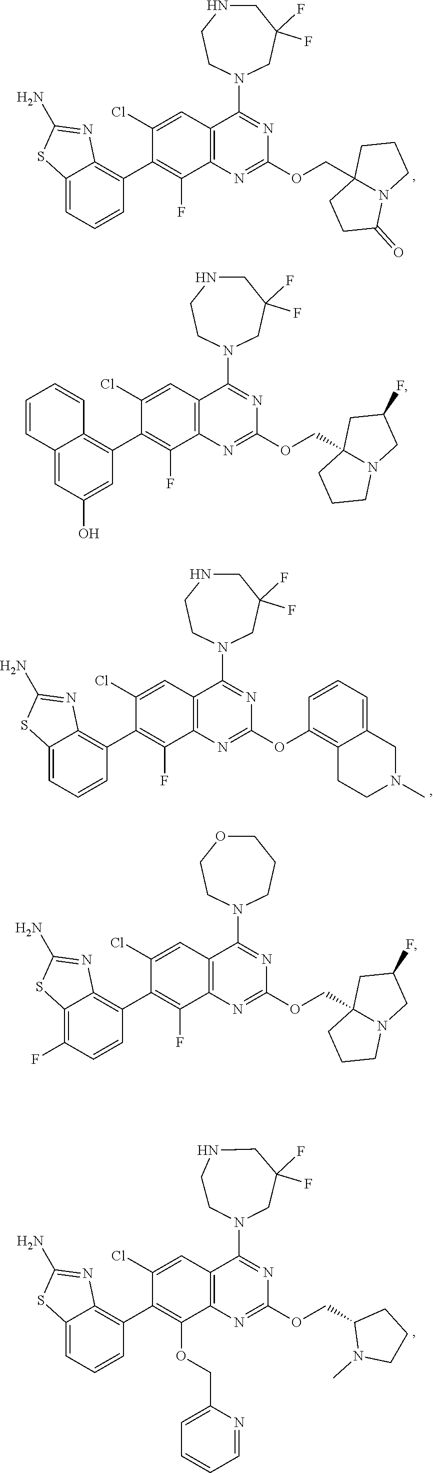 [SMILES: Nc1nc2c(-c3c(Cl)cc4c(N5CCNCC(F)(F)C5)nc(OCC56CCCN5C(=O)CC6)nc4c3F)cccc2s1 ; Oc1cc(-c2c(Cl)cc3c(N4CCNCC(F)(F)C4)nc(OC[C@@]45CCCN4C[C@H](F)C5)nc3c2F)c2ccccc2c1 ; CN1CCc2c(cccc2Oc2nc(N3CCNCC(F)(F)C3)c3cc(Cl)c(-c4cccc5sc(N)nc45)c(F)c3n2)C1 ; Nc1nc2c(-c3c(Cl)cc4c(N5CCCOCC5)nc(OC[C@@]56CCCN5C[C@H](F)C6)nc4c3F)ccc(F)c2s1 ; CN1CCC[C@H]1COc1nc(N2CCNCC(F)(F)C2)c2cc(Cl)c(-c3cccc4sc(N)nc34)c(OCc3ccccn3)c2n1]

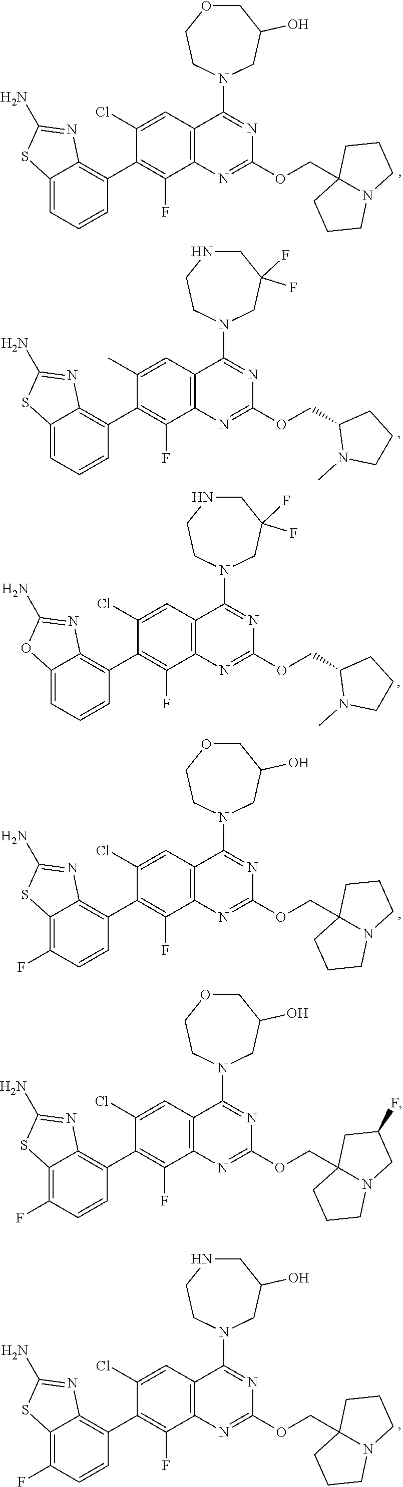

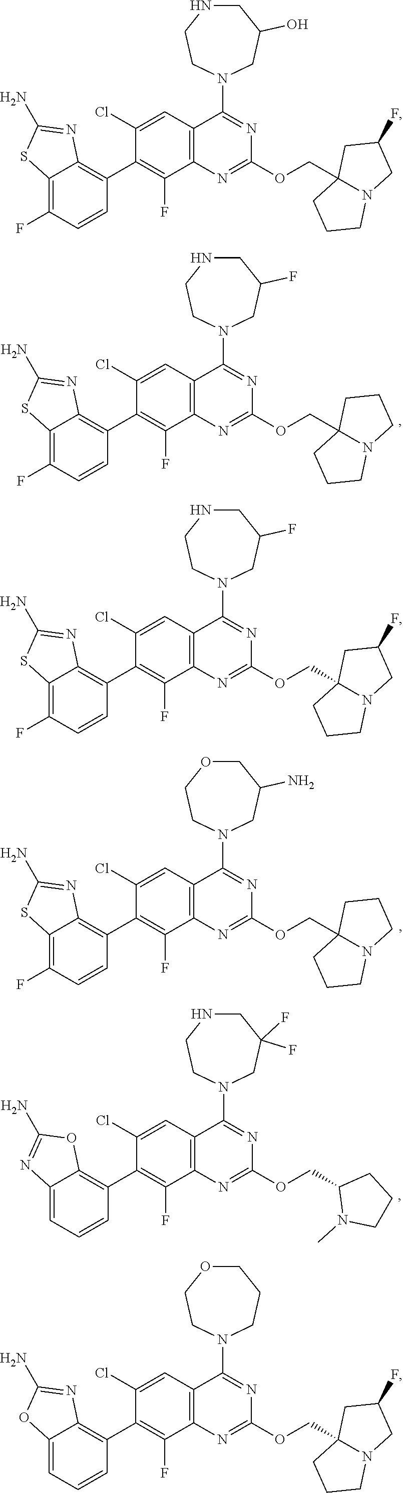 [SMILES: Nc1nc2c(-c3c(Cl)cc4c(N5CCNCC(O)C5)nc(OCC56CCCN5C[C@H](F)C6)nc4c3F)ccc(F)c2s1 ; Nc1nc2c(-c3c(Cl)cc4c(N5CCNCC(F)C5)nc(OCC56CCCN5CCC6)nc4c3F)ccc(F)c2s1 ; Nc1nc2c(-c3c(Cl)cc4c(N5CCNCC(F)C5)nc(OC[C@@]56CCCN5C[C@H](F)C6)nc4c3F)ccc(F)c2s1 ; Nc1nc2c(-c3c(Cl)cc4c(N5CCOCC(N)C5)nc(OCC56CCCN5CCC6)nc4c3F)ccc(F)c2s1 ; CN1CCC[C@H]1COc1nc(N2CCNCC(F)(F)C2)c2cc(Cl)c(-c3cccc4nc(N)oc34)c(F)c2n1 ; Nc1nc2c(-c3c(Cl)cc4c(N5CCCOCC5)nc(OC[C@@]56CCCN5C[C@H](F)C6)nc4c3F)cccc2o1]

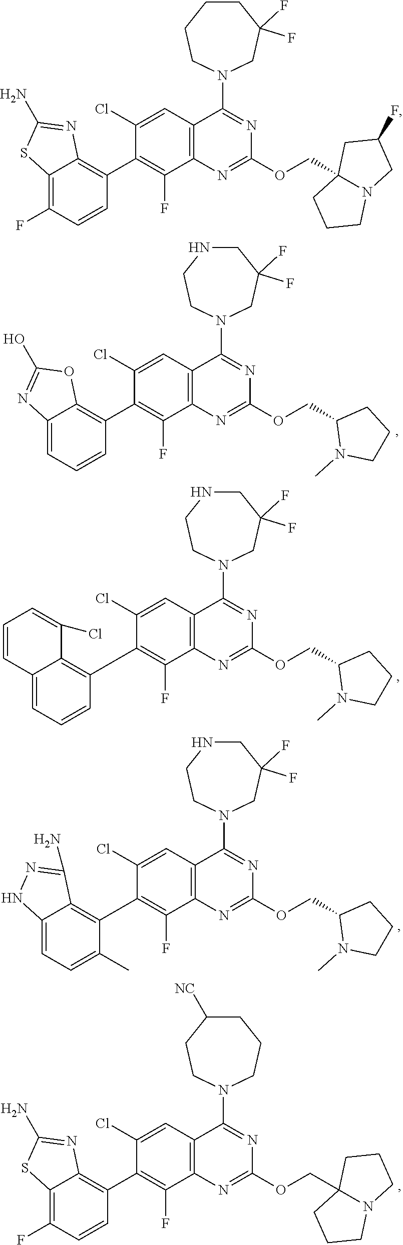

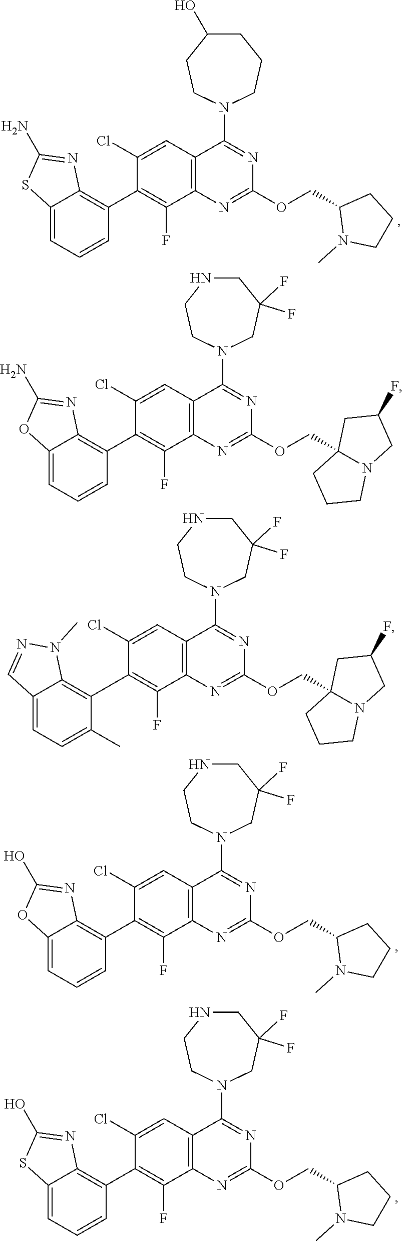

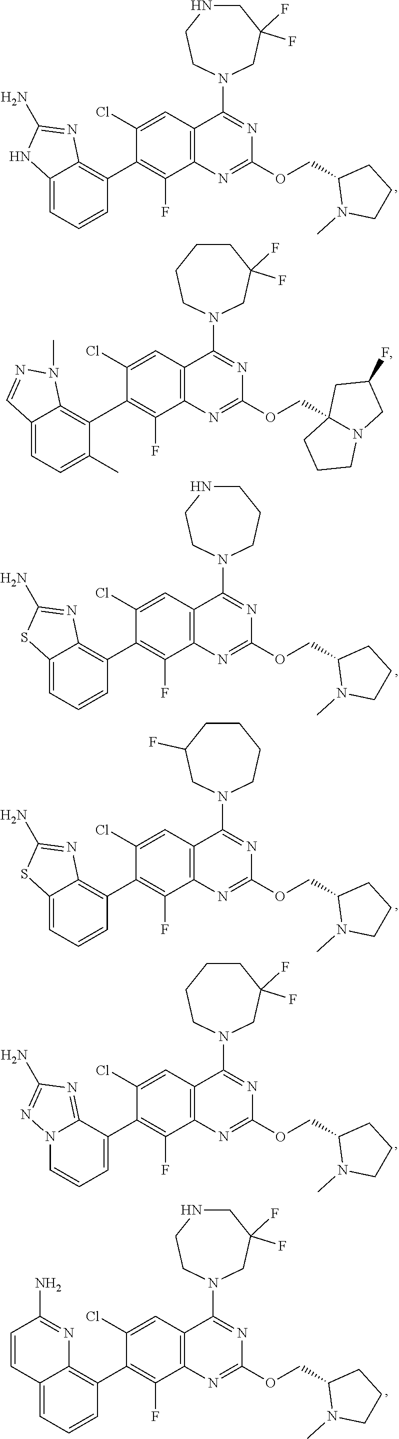

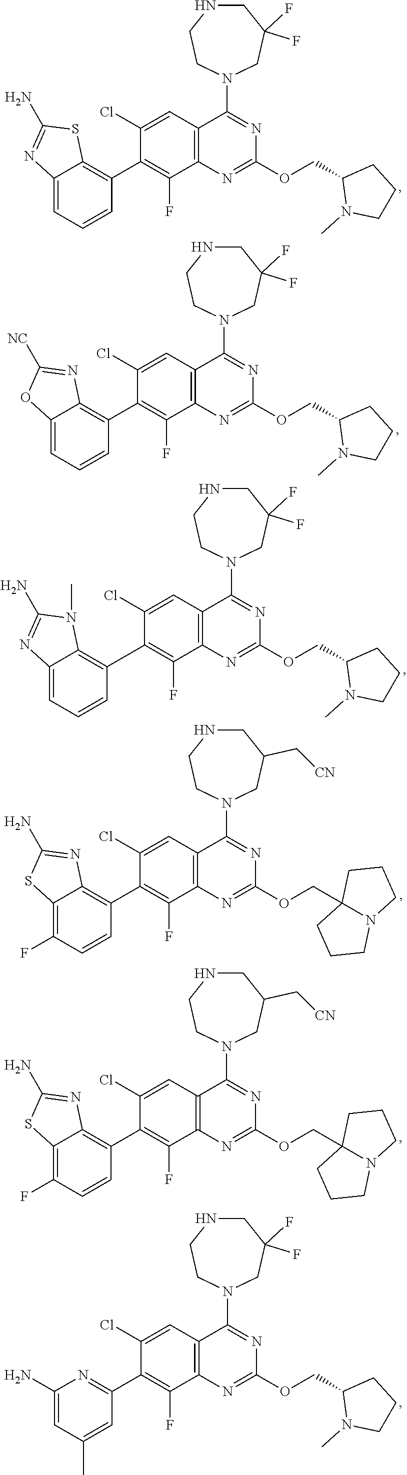

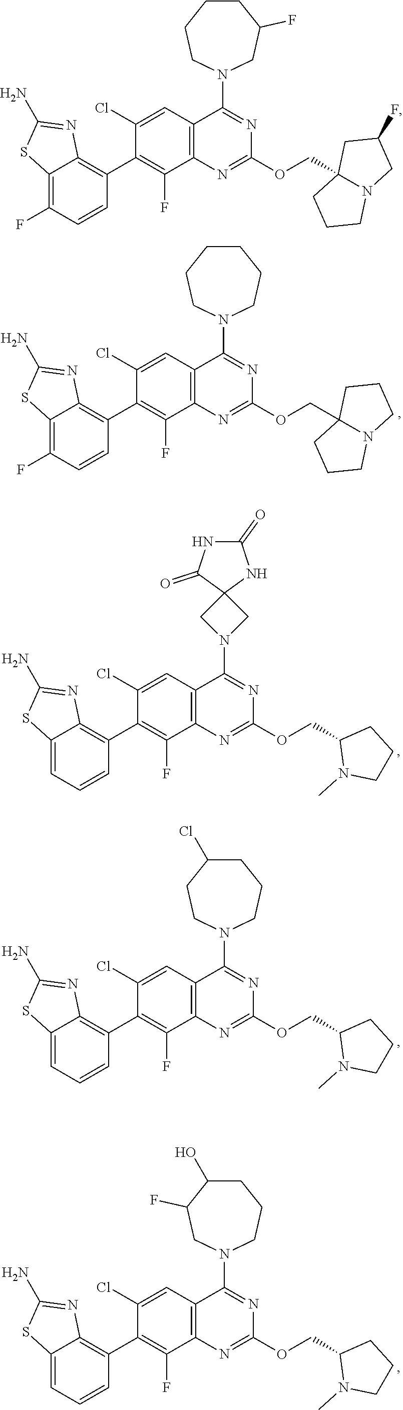 [SMILES: Nc1nc2c(-c3c(Cl)cc4c(N5CCCCC(F)C5)nc(OC[C@@]56CCCN5C[C@H](F)C6)nc4c3F)ccc(F)c2s1 ; Nc1nc2c(-c3c(Cl)cc4c(N5CCCCCC5)nc(OCC56CCCN5CCC6)nc4c3F)ccc(F)c2s1 ; CN1CCC[C@H]1COc1nc(N2CC3(C2)NC(=O)NC3=O)c2cc(Cl)c(-c3cccc4sc(N)nc34)c(F)c2n1 ; CN1CCC[C@H]1COc1nc(N2CCCC(Cl)CC2)c2cc(Cl)c(-c3cccc4sc(N)nc34)c(F)c2n1 ; CN1CCC[C@H]1COc1nc(N2CCCC(O)C(F)C2)c2cc(Cl)c(-c3cccc4sc(N)nc34)c(F)c2n1]

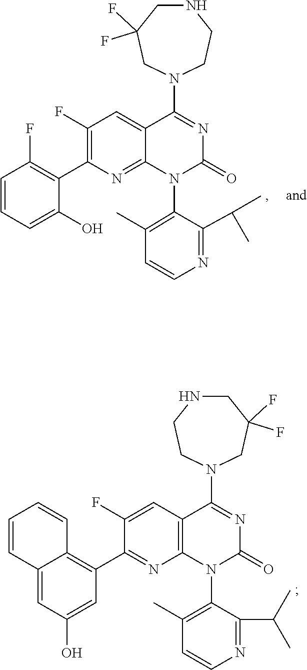 [SMILES: Cc1ccnc(C(C)C)c1-n1c(=O)nc(N2CCNCC(F)(F)C2)c2cc(F)c(-c3c(O)cccc3F)nc21 ; Cc1ccnc(C(C)C)c1-n1c(=O)nc(N2CCNCC(F)(F)C2)c2cc(F)c(-c3cc(O)cc4ccccc34)nc21]

or a pharmaceutically acceptable salt or solvate thereof.

Embodiment 140. A pharmaceutical composition comprising a compound of any one of embodiments 1-139, or a pharmaceutically acceptable salt or solvate thereof, and a pharmaceutically acceptable excipient.

Embodiment 141. A method of treating cancer in a subject in need thereof, comprising administering to the subject a therapeutically effective amount of a compound of any one of embodiments 1-139, or a pharmaceutically acceptable salt or solvate thereof.

Embodiment 142. The method of embodiment 141, wherein the cancer is a solid tumor.

Embodiment 143. The method of embodiment 141, wherein the cancer is a hematological cancer.

Embodiment 144. A method of modulating activity of a Ras protein, comprising contacting a Ras protein with an effective amount of a compound of any one of embodiments 1-139, or a pharmaceutically acceptable salt or solvate thereof, thereby modulating the activity of the Ras protein.

Embodiment 145. The method of embodiment 144, wherein said modulating comprises inhibiting the Ras protein activity.

Embodiment 146. The method of embodiment 144, wherein the Ras protein is a K-Ras protein.

Embodiment 147. The method of embodiment 144, wherein the Ras protein is a G12D or G12V mutant K-Ras.

Embodiment 148. The method of any one of embodiments 141-147 comprising administering an additional agent or therapy.

Embodiment 149. The method of embodiment 148, wherein the additional agent or therapy is selected from the group consisting of a chemotherapeutic agent, a radioactive agent, and an immune modulator.

Embodiment 150. The method of embodiment 144, wherein said modulating takes place in vitro or in vivo.

Embodiment 151. A method of inhibiting cell growth, comprising administering a cell expressing a Ras protein with an effective amount of a compound of any one of embodiments 1-139, or a pharmaceutically acceptable salt or solvate thereof, thereby inhibiting growth of said cells.

Embodiment 152. The method of embodiment 151 comprising administering an additional agent to said cell.

Embodiment 153. The method of embodiment 152, wherein the additional agent is a chemotherapeutic agent, a radioactive agent, or an immune modulator.

Embodiment 154. A Ras protein bound by a compound of any one of embodiments 1-139, or a pharmaceutically acceptable salt or solvate thereof, wherein activity of said Ras protein is reduced as compared to a Ras protein unbound to said compound.

Embodiment A1. A compound of Formula (I), or a pharmaceutically acceptable salt or solvate thereof:

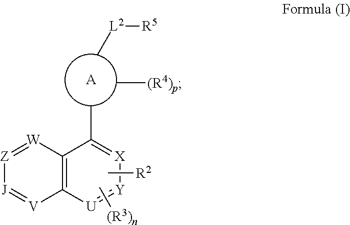

wherein:

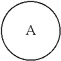

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, Co-10aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};

R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment A2 The compound of Embodiment A1, or a pharmaceutically acceptable salt or solvate thereof, having the structure of Formula (I′):

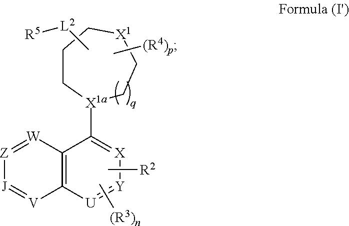

wherein X¹ is selected from C, N, O, S, S(O), and S(O)₂; X^{1a} is selected from N and C(H); and q is 1 or 2.

Embodiment A3 The compound of Embodiment A1 or Embodiment A2, or a pharmaceutically acceptable salt or solvate thereof, having the structure of Formula (Ia):

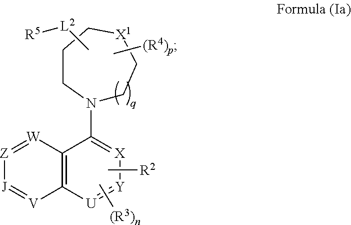

wherein X¹ is selected from C, N, O, S, S(O), and S(O)₂; and q is 1 or 2.

Embodiment A4 The compound of Embodiment A3, or a pharmaceutically acceptable salt or solvate thereof, having the structure of Formula (Ib):

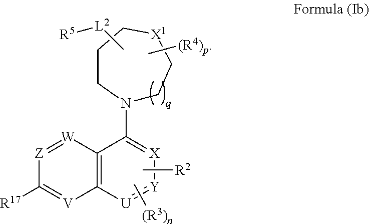

Embodiment A5 The compound of Embodiment A4, or a pharmaceutically acceptable salt or solvate thereof, having the structure of Formula (Ic):

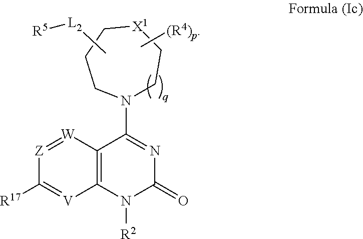

Embodiment A6 The compound of Embodiment A4, or a pharmaceutically acceptable salt or solvate thereof, having the structure of Formula (Id):

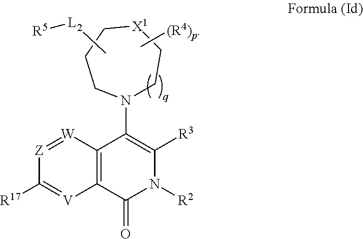

Embodiment A7 The compound of Embodiment A4, or a pharmaceutically acceptable salt or solvate thereof, having the structure of Formula (Ie):

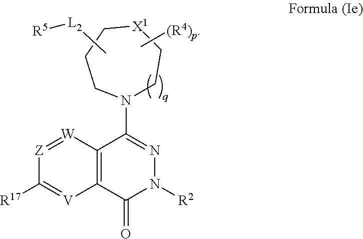

Embodiment A8 The compound of Embodiment A4, or a pharmaceutically acceptable salt or solvate thereof, having the structure of Formula (If):

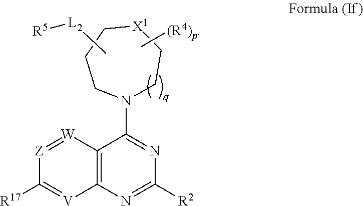

Embodiment A9 The compound of Embodiment A4, or a pharmaceutically acceptable salt or solvate thereof, having the structure of Formula (Ig):

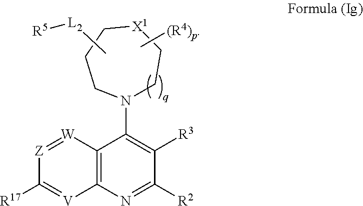

Embodiment A10 A compound of Formula (I″), or a pharmaceutically acceptable salt or solvate thereof:

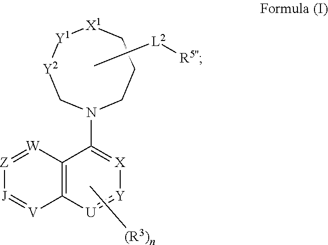

wherein:
X is C or N;
X¹ is selected from C(R*)(R⁴), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁴);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}.

R⁵ is an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and
[symbol] indicates a single or double bond such that all valences are satisfied.

Embodiment A11 The compound of Embodiment A10, or a pharmaceutically acceptable salt or solvate thereof, wherein Y² is a bond.

Embodiment A12 The compound of Embodiment A10, or a pharmaceutically acceptable salt or solvate thereof, wherein Y² is CH₂.

Embodiment A13 The compound of any one of Embodiments A10-A12, or a pharmaceutically acceptable salt or solvate thereof, wherein Y¹ is CH₂.

Embodiment A14 The compound of any one of Embodiments A2-A13, or a pharmaceutically acceptable salt or solvate thereof, wherein X¹ is N(H).

Embodiment A15 The compound of any one of Embodiments A2-A13, or a pharmaceutically acceptable salt or solvate thereof, wherein X¹ is N(R⁴).

Embodiment A16 The compound of any one of Embodiments A2-A13, or a pharmaceutically acceptable salt or solvate thereof, wherein X¹ is N(-L²-R⁵).

Embodiment A17 The compound of any one of Embodiments A2-A13, or a pharmaceutically acceptable salt or solvate thereof, wherein X¹ is O.

Embodiment A18 The compound of any one of Embodiments A2-A13, or a pharmaceutically acceptable salt or solvate thereof, wherein X¹ is S.

Embodiment A19 The compound of any one of Embodiments A2-A13, or a pharmaceutically acceptable salt or solvate thereof, wherein X¹ is S(O)₂.

Embodiment A20 The compound of any one of Embodiments A2-A13, or a pharmaceutically acceptable salt or solvate thereof, wherein X¹ is CH₂.

Embodiment A21 The compound of any one of Embodiments A2-A13, or a pharmaceutically acceptable salt or solvate thereof, wherein X¹ is C(H)(R⁴).

Embodiment A22 The compound of any one of Embodiments A2-A13, or a pharmaceutically acceptable salt or solvate thereof, wherein X¹ is C(R⁴)₂.

Embodiment A23 The compound of any one of Embodiments A2-A13, or a pharmaceutically acceptable salt or solvate thereof, wherein X¹ is C(H)(-L²-R⁵).

Embodiment A24 The compound of any one of Embodiments A2-A23, or a pharmaceutically acceptable salt or solvate thereof, wherein q is 1.

Embodiment A25 The compound of any one of Embodiments A2-A23, or a pharmaceutically acceptable salt or solvate thereof, wherein q is 2.

Embodiment A26 The compound of any one of Embodiments A1-A25, or a pharmaceutically acceptable salt or solvate thereof, wherein V is C(R¹⁶).

Embodiment A27 The compound of any one of Embodiments A1-A26, or a pharmaceutically acceptable salt or solvate thereof, wherein V is C(H).

Embodiment A28 The compound of any one of Embodiments A1-A25, or a pharmaceutically acceptable salt or solvate thereof, wherein V is N.

Embodiment A29 The compound of any one of Embodiments A1-A28, or a pharmaceutically acceptable salt or solvate thereof, wherein W is C(R¹⁸).

Embodiment A30 The compound of Embodiment A29, or a pharmaceutically acceptable salt or solvate thereof, wherein W is C(H).

Embodiment A31 The compound of any one of Embodiments A1-A28, or a pharmaceutically acceptable salt or solvate thereof, wherein W is N.

Embodiment A32 The compound of any one of Embodiments A1-A31, or a pharmaceutically acceptable salt or solvate thereof, wherein Z is C(R⁸).

Embodiment A33 The compound of Embodiment A32, or a pharmaceutically acceptable salt or solvate thereof, wherein Z is C(H).

Embodiment A34 The compound of any one of Embodiments A1-A31, or a pharmaceutically acceptable salt or solvate thereof, wherein Z is N.

Embodiment A35 A compound of Formula (II), or a pharmaceutically acceptable salt or solvate thereof:

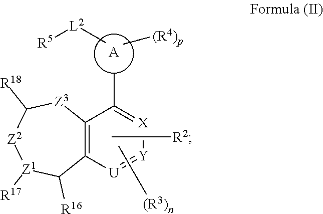

wherein:

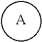

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸); Z¹ is N or C(R⁶); Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

Embodiment A36 The compound of Embodiment A35, or a pharmaceutically acceptable salt or solvate thereof, having the structure of Formula (IIa):

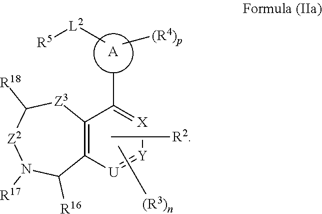

Embodiment A37 The compound of Embodiment A35, or a pharmaceutically acceptable salt or solvate thereof, having the structure of Formula (IIb):

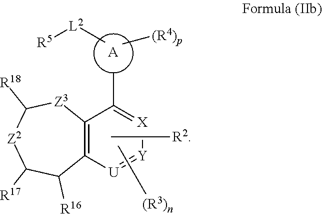

Embodiment A38 The compound of Embodiment A35 or Embodiment A36, or a pharmaceutically acceptable salt or solvate thereof, having the structure of Formula (IIc′):

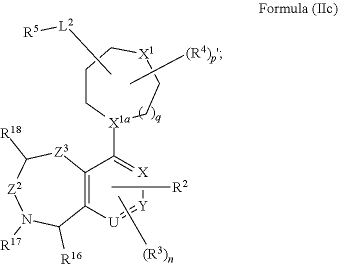

wherein X¹ is selected from C, N, O, S, S(O), and S(O)₂; X^{1a} is selected from N and C(H); and q is 1 or 2.

Embodiment A39 The compound of Embodiment A38, or a pharmaceutically acceptable salt or solvate thereof, having the structure of Formula (IIc):

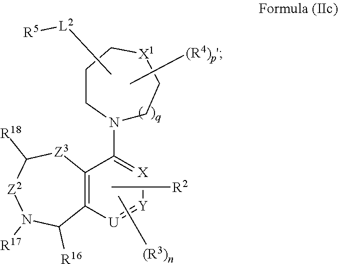

wherein X¹ is selected from C, N, O, S, S(O), and S(O)₂; and q is 1 or 2.

Embodiment A40 The compound of Embodiment A35 or Embodiment A37, or a pharmaceutically acceptable salt or solvate thereof, having the structure of Formula (IId′):

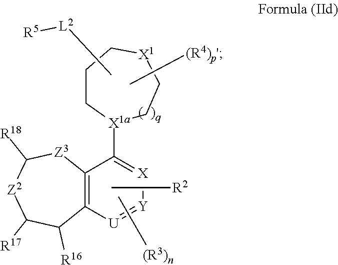

wherein X¹ is selected from C, N, O, S, S(O), and S(O)₂; X^{1a} is selected from N and C(H); and q is 1 or 2.

Embodiment A41 The compound of Embodiment A40, or a pharmaceutically acceptable salt or solvate thereof, having the structure of Formula (IId):

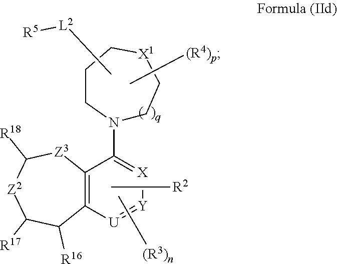

wherein X¹ is selected from C, N, O, S, S(O), and S(O)₂; and q is 1 or 2.

Embodiment A42 The compound of any one of Embodiments A38-A41, or a pharmaceutically acceptable salt or solvate thereof, wherein X¹ is N(H).

Embodiment A43 The compound of any one of Embodiments A38-A41, or a pharmaceutically acceptable salt or solvate thereof, wherein X¹ is N(R⁴).

Embodiment A44 The compound of any one of Embodiments A38-A41, or a pharmaceutically acceptable salt or solvate thereof, wherein X¹ is N(-L²-R⁵).

Embodiment A45 The compound of any one of Embodiments A38-A41, or a pharmaceutically acceptable salt or solvate thereof, wherein X¹ is O.

Embodiment A46 The compound of any one of Embodiments A38-A41, or a pharmaceutically acceptable salt or solvate thereof, wherein X¹ is S.

Embodiment A47 The compound of any one of Embodiments A38-A41, or a pharmaceutically acceptable salt or solvate thereof, wherein X¹ is S(O)₂.

Embodiment A48 The compound of any one of Embodiments A38-A41, or a pharmaceutically acceptable salt or solvate thereof, wherein X¹ is CH₂.

Embodiment A49 The compound of any one of Embodiments A38-A41, or a pharmaceutically acceptable salt or solvate thereof, wherein X¹ is C(H)(R⁴).

Embodiment A50 The compound of any one of Embodiments A38-A41, or a pharmaceutically acceptable salt or solvate thereof, wherein X¹ is C(R⁴)₂.

Embodiment A51 The compound of any one of Embodiments A38-A41, or a pharmaceutically acceptable salt or solvate thereof, wherein X¹ is C(H)(-L²-R⁵).

Embodiment A52 The compound of any one of Embodiments A of any one of Embodiments A38-A51, or a pharmaceutically acceptable salt or solvate thereof, wherein q is 1.

Embodiment A53 The compound of any one of Embodiments A38-A51, or a pharmaceutically acceptable salt or solvate thereof, wherein q is 2.

Embodiment A54 The compound of any one of Embodiments A35-A53, or a pharmaceutically acceptable salt or solvate thereof, wherein Z³ is absent.

Embodiment A55 The compound of any one of Embodiments A35-A53, or a pharmaceutically acceptable salt or solvate thereof, wherein Z² is C(R⁸)(R⁹).

Embodiment A56 The compound of any one of Embodiments A35-A55, or a pharmaceutically acceptable salt or solvate thereof, wherein R⁹ is hydrogen.

Embodiment A57 The compound of any one of Embodiments A35-A55, or a pharmaceutically acceptable salt or solvate thereof, wherein X is C.

Embodiment A58 The compound of any one of Embodiments A35-A55, or a pharmaceutically acceptable salt or solvate thereof, wherein X is N.

Embodiment A58 The compound of any one of Embodiments A35-A58, or a pharmaceutically acceptable salt or solvate thereof, wherein Y is C.

Embodiment A60 The compound of any one of Embodiments A35-A58, or a pharmaceutically acceptable salt or solvate thereof, wherein Y is N.

Embodiment A61 The compound of any one of Embodiments A35-A58, or a pharmaceutically acceptable salt or solvate thereof, wherein Y is C(O).

Embodiment A62 The compound of any one of Embodiments A35-A61, or a pharmaceutically acceptable salt or solvate thereof, wherein U is C.

Embodiment A63 The compound of any one of Embodiments A35-A61, or a pharmaceutically acceptable salt or solvate thereof, wherein U is N.

Embodiment A64 The compound of any one of Embodiments A35-A60, or a pharmaceutically acceptable salt or solvate thereof, wherein U is C(O).

Embodiment A65 The compound of any one of Embodiments A42-A53, or a pharmaceutically acceptable salt or solvate thereof, having the structure of Formula (IIe):

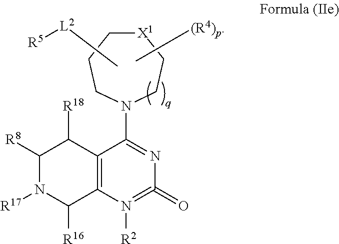

Embodiment A66 The compound of any one of Embodiments A42-A53, or a pharmaceutically acceptable salt or solvate thereof, having the structure of Formula (IIf):

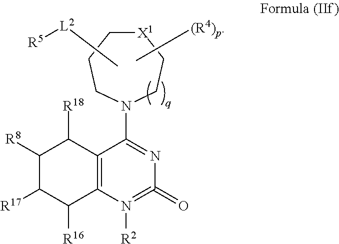

Embodiment A67 The compound of any one of Embodiments A42-A53, or a pharmaceutically acceptable salt or solvate thereof, having the structure of Formula (IIg):

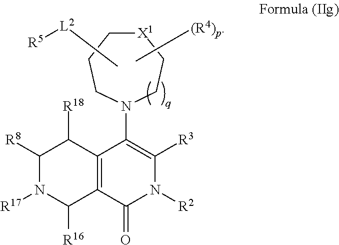

Embodiment A68 The compound of any one of Embodiments A42-A53, or a pharmaceutically acceptable salt or solvate thereof, having the structure of Formula (IIh):

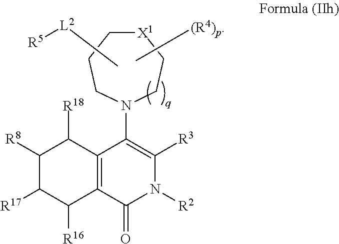

Embodiment A69 The compound of any one of Embodiments A42-A53, or a pharmaceutically acceptable salt or solvate thereof, having the structure of Formula (IIi):

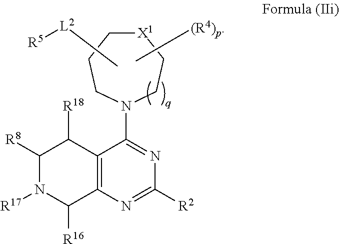

Embodiment A70 The compound of any one of Embodiments A42-A53, or a pharmaceutically acceptable salt or solvate thereof, having the structure of Formula (IIj):

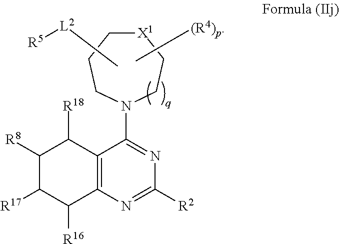

Embodiment A71 The compound of any one of Embodiments A42-A53, or a pharmaceutically acceptable salt or solvate thereof, having the structure of Formula (IIk):

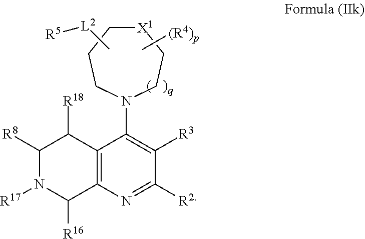

Embodiment A72 The compound of any one of Embodiments A42-A53, or a pharmaceutically acceptable salt or solvate thereof, having the structure of Formula (IIm):

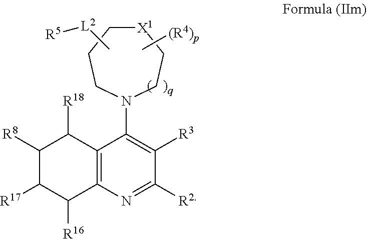

Embodiment A73 A compound of Formula (IIIa)-(IIIi), or a pharmaceutically acceptable salt or solvate thereof:

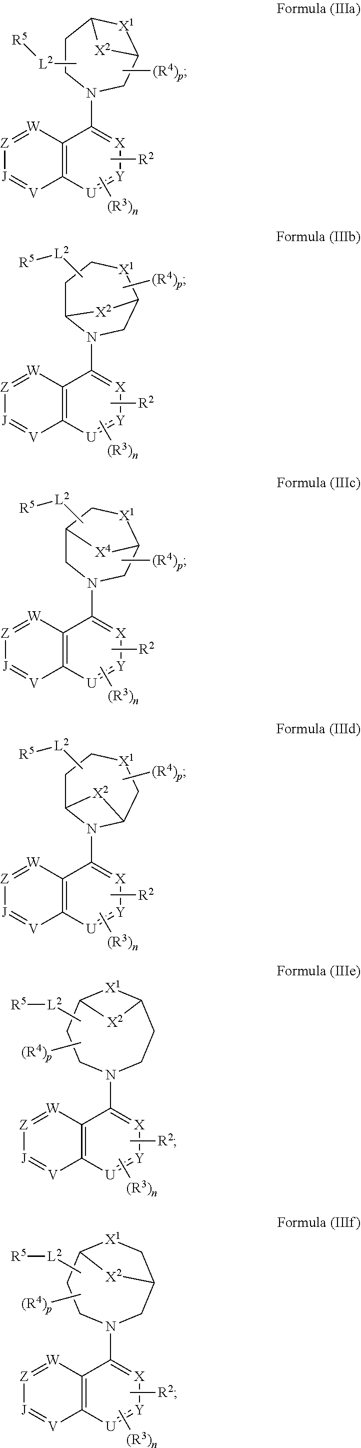

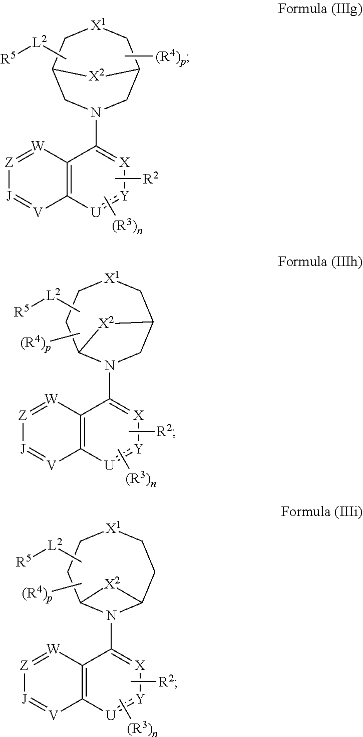

wherein:
X is C or N;
X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X⁵, —CH₂—, —X⁵CH₂—, —CH₂X³—, —CH₂CH₂—, —C(H)(R⁴)—, —C(H)(R⁴)—, —X⁵—, —C(H)(R⁴)—, —C(H)(R⁴)X³—, —C(H)(R⁴)CH₂—, —C(H)(R⁴)C(H)(R⁴)—, —C(R⁴)₂—, —X⁵—, —C(R⁴)₂—, —C(R⁴)₂X³—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁶ is -L²-R⁵;

R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment A74 The compound of Embodiment A73, or a pharmaceutically acceptable salt or solvate thereof, having the structure of Formula (IIIa):

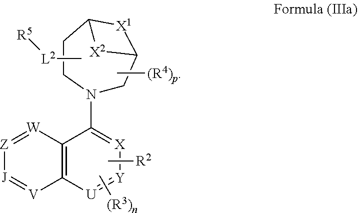

Embodiment A75 The compound of Embodiment A73, or a pharmaceutically acceptable salt or solvate thereof, having the structure of Formula (IIIb):

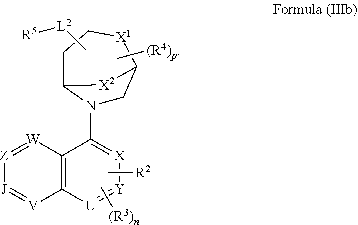

Embodiment A76 The compound of Embodiment A73, or a pharmaceutically acceptable salt or solvate thereof, having the structure of Formula (IIId):

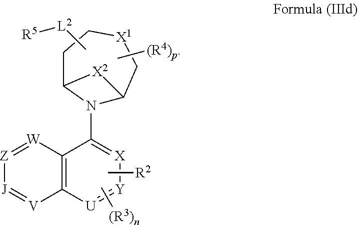

Embodiment A77 The compound of Embodiment A73, or a pharmaceutically acceptable salt or solvate thereof, having the structure of Formula (IIIe):

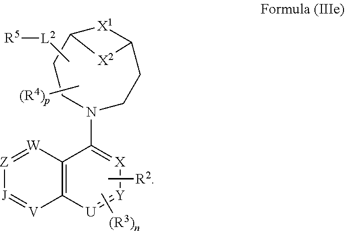

Embodiment A78 The compound of Embodiment A73, or a pharmaceutically acceptable salt or solvate thereof, having the structure of Formula (IIIf):

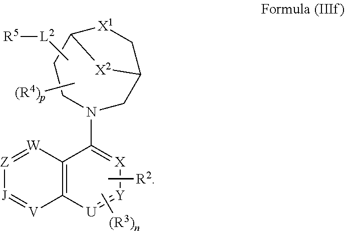

Embodiment A79 The compound of Embodiment A73, or a pharmaceutically acceptable salt or solvate thereof, having the structure of Formula (IIIg):

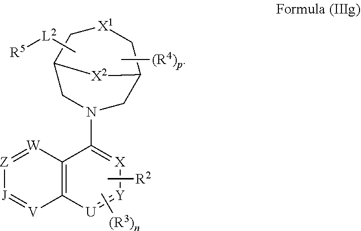

Embodiment A80 The compound of Embodiment A73, or a pharmaceutically acceptable salt or solvate thereof, having the structure of Formula (IIIh):

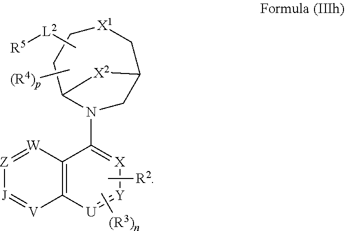

Embodiment A81 The compound of Embodiment A73, or a pharmaceutically acceptable salt or solvate thereof, having the structure of Formula (IIIi):

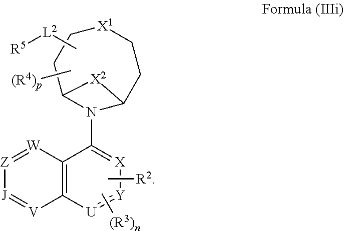

Embodiment A82 The compound of Embodiment A73, or a pharmaceutically acceptable salt or solvate thereof, having the structure of Formula (IIIc):

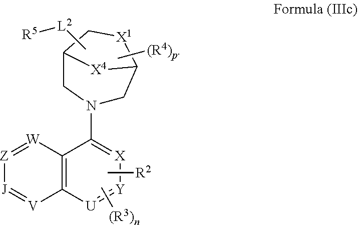

Embodiment A83 The compound of Embodiment A82, or a pharmaceutically acceptable salt or solvate thereof, wherein X⁴ is selected from —CH₂— and —CH₂CH₂—.

Embodiment A84 The compound of any one of Embodiments A73-A81, or a pharmaceutically acceptable salt or solvate thereof, wherein X² is selected from —CH₂— and —CH₂CH₂—.

Embodiment A85 A compound of Formula (IVa), (IVb), or (IVc), or a pharmaceutically acceptable salt or solvate thereof:

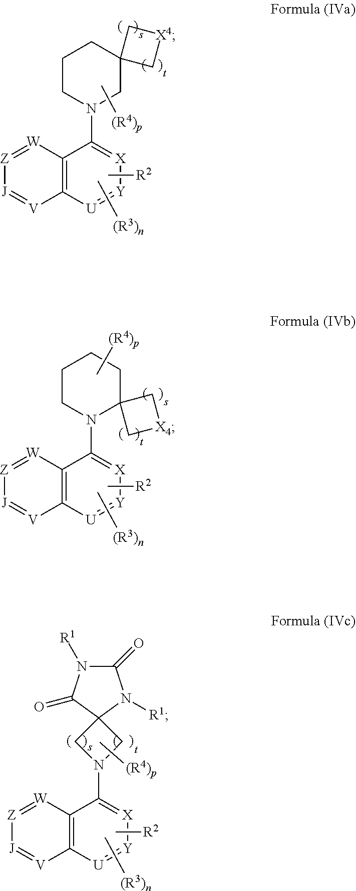

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20b}; R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, Co-10aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁-heteroaryl are optionally substituted with one, two, or three groups independently selected from halogen, oxo, —CN, C₁₋₆alkyl, C₁-haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

Embodiment A86 The compound of Embodiment A85, or a pharmaceutically acceptable salt or solvate thereof, having the structure of Formula (IVa):

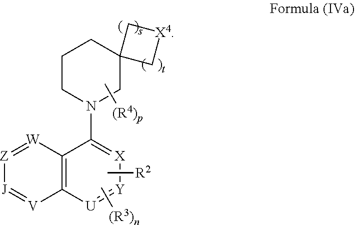

Embodiment A87 The compound of Embodiment A85, or a pharmaceutically acceptable salt or solvate thereof, having the structure of Formula (IVb):

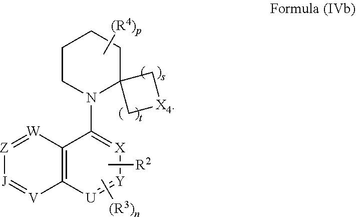

Embodiment A88 The compound of any one of Embodiments A85-A87, or a pharmaceutically acceptable salt or solvate thereof, wherein s is 1 or 2.

Embodiment A89 The compound of any one of Embodiments A85-A88, or a pharmaceutically acceptable salt or solvate thereof, wherein t is 1 or 2.

Embodiment A90 The compound of any one of Embodiments A85-A89, or a pharmaceutically acceptable salt or solvate thereof, wherein X⁴ is N(R¹).

Embodiment A91 The compound of Embodiment A85, or a pharmaceutically acceptable salt or solvate thereof, having the structure of Formula (IVc):

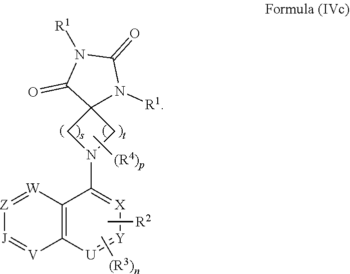

Embodiment A92 A compound of Formula (Va), (Vb), or (Vc), or a pharmaceutically acceptable salt or solvate thereof:

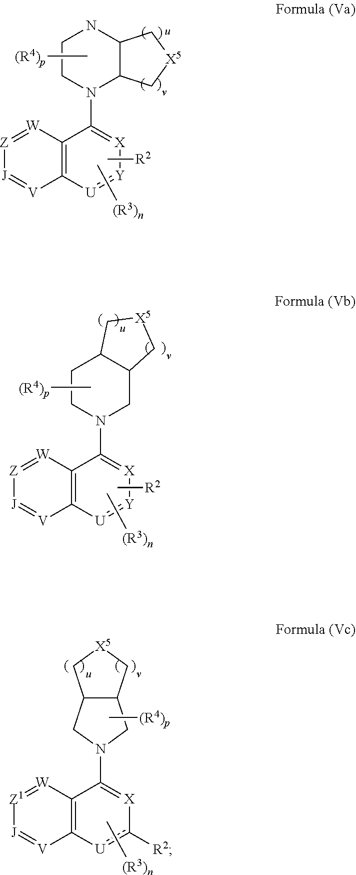

wherein:
X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—; R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with the cysteine residue at position 12 of a KRAS protein; R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁-heteroaryl are optionally substituted with one, two, or three groups independently selected from halogen, oxo, —CN, C₁₋₆alkyl, C₁-haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

Embodiment A93 The compound of Embodiment A92, or a pharmaceutically acceptable salt or solvate thereof, having the structure of Formula (Va):

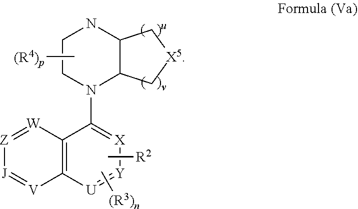

Embodiment A94 The compound of Embodiment A92, or a pharmaceutically acceptable salt or solvate thereof, having the structure of Formula (Vb):

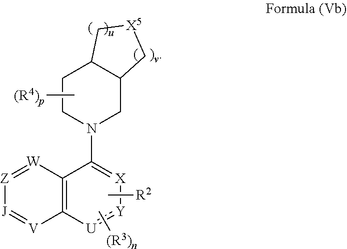

Embodiment A95 The compound of Embodiment A92, or a pharmaceutically acceptable salt or solvate thereof, having the structure of Formula (Vc):

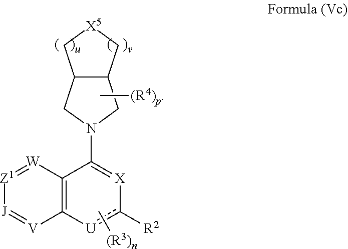

Embodiment A96 The compound of any one of Embodiments A92-A95, or a pharmaceutically acceptable salt or solvate thereof, wherein u is 0 or 1.

Embodiment A97 The compound of any one of Embodiments A92-A95, or a pharmaceutically acceptable salt or solvate thereof, wherein v is 0 or 1.

Embodiment A98 The compound of any one of Embodiments A92-A97, or a pharmaceutically acceptable salt or solvate thereof, wherein X⁵ is N(R¹).

Embodiment A99 The compound of any one of Embodiments A85-A98, or a pharmaceutically acceptable salt or solvate thereof, wherein R¹ is hydrogen.

Embodiment A100 The compound of any one of Embodiments A84-A98, or a pharmaceutically acceptable salt or solvate thereof, wherein R¹ is -L²-R⁵.

Embodiment A101 The compound of any one of Embodiments A73-A100, or a pharmaceutically acceptable salt or solvate thereof, wherein Y is C.

Embodiment A102 The compound of any one of Embodiments A73-A100, or a pharmaceutically acceptable salt or solvate thereof, wherein Y is N.

Embodiment A103 The compound of any one of Embodiments A73-A100, or a pharmaceutically acceptable salt or solvate thereof, wherein Y is C(O).

Embodiment A104 The compound of any one of Embodiments A73-A103, or a pharmaceutically acceptable salt or solvate thereof, wherein X is C.

Embodiment A105 The compound of any one of Embodiments A73-A103, or a pharmaceutically acceptable salt or solvate thereof, wherein X is N.

Embodiment A106 The compound of any one of Embodiments A73-A105, or a pharmaceutically acceptable salt or solvate thereof, wherein U is C.

Embodiment A107 The compound of any one of Embodiments A73-A105, or a pharmaceutically acceptable salt or solvate thereof, wherein U is N.

Embodiment A108 The compound of any one of Embodiments A73-A105, or a pharmaceutically acceptable salt or solvate thereof, wherein U is C(O).

Embodiment A109 The compound of any one of Embodiments A73-A108, or a pharmaceutically acceptable salt or solvate thereof, wherein Z is C(R⁸).

Embodiment A110 The compound of any one of Embodiments A73-A109, or a pharmaceutically acceptable salt or solvate thereof, wherein R⁸ is hydrogen.

Embodiment A111 The compound of any one of Embodiments A73-A108, or a pharmaceutically acceptable salt or solvate thereof, wherein Z is N.

Embodiment A112 The compound of any one of Embodiments A73-A111, or a pharmaceutically acceptable salt or solvate thereof, wherein V is C(R¹⁶).

Embodiment A113 The compound of any one of Embodiments A73-A112, or a pharmaceutically acceptable salt or solvate thereof, wherein V is C(H).

Embodiment A114 The compound of any one of Embodiments A73-A111, or a pharmaceutically acceptable salt or solvate thereof, wherein V is N.

Embodiment A115 The compound of any one of Embodiments A73-A114, or a pharmaceutically acceptable salt or solvate thereof, wherein J is C(R¹⁷).

Embodiment A116 The compound of any one of Embodiments A73-A115, or a pharmaceutically acceptable salt or solvate thereof, wherein W is C(R¹⁸).

Embodiment A117 The compound of Embodiment A116, or a pharmaceutically acceptable salt or solvate thereof, wherein W is C(H).

Embodiment A118 The compound of any one of Embodiments A73-A117, or a pharmaceutically acceptable salt or solvate thereof, wherein W is N.

Embodiment A119 The compound of any one of Embodiments A1-A118, or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}.

Embodiment A120 The compound of any one of Embodiments A1-A119, or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}.

Embodiment A121 The compound of any one of Embodiments A1-A120, or a pharmaceutically acceptable salt or solvate thereof, wherein R² is —OR¹².

Embodiment A122 The compound of any one of Embodiments A1-A121, or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}.

Embodiment A123 The compound of any one of Embodiments A1-A121, or a pharmaceutically acceptable salt or solvate thereof, wherein R¹² is C₁₋₆alkyl optionally substituted with one, two, or three R^{20d}.

Embodiment A124 The compound of any one of Embodiments A1-A121, or a pharmaceutically acceptable salt or solvate thereof, wherein R¹² is C₂₋₉heterocycloalkyl optionally substituted with one, two, or three R^{20d}.

Embodiment A125 The compound of any one of Embodiments A1-A121, or a pharmaceutically acceptable salt or solvate thereof, wherein R¹² is —CH₂—C₂₋₉heterocycloalkyl optionally substituted with one, two, or three R^{20d}.

Embodiment A126 The compound of any one of Embodiments A1-A125, or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³).

Embodiment A127 The compound of any one of Embodiments A1-A126, or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

Embodiment A128 The compound of any one of Embodiments A1-A119, or a pharmaceutically acceptable salt or solvate thereof, wherein R² is selected from

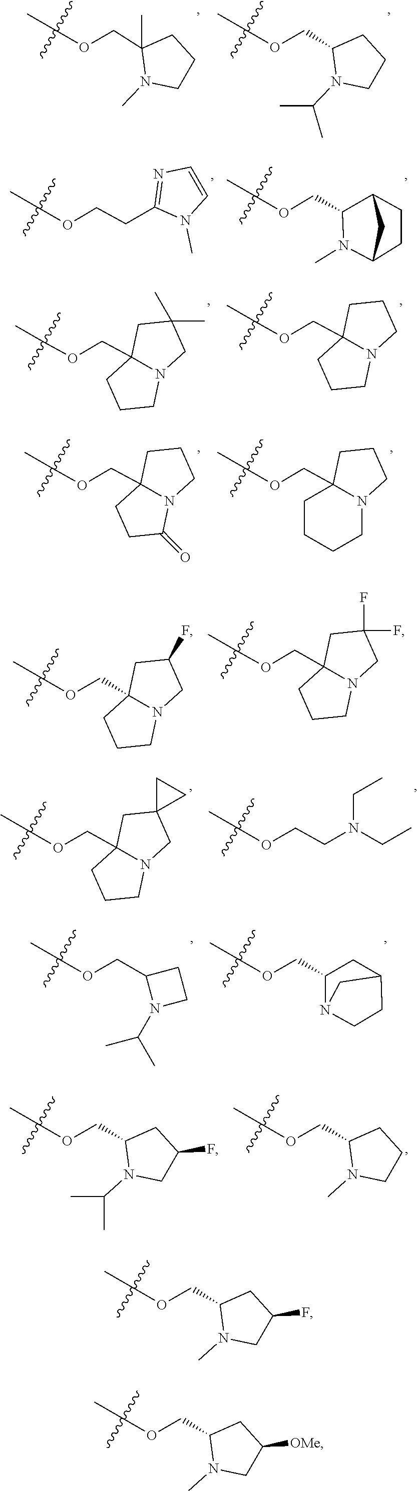

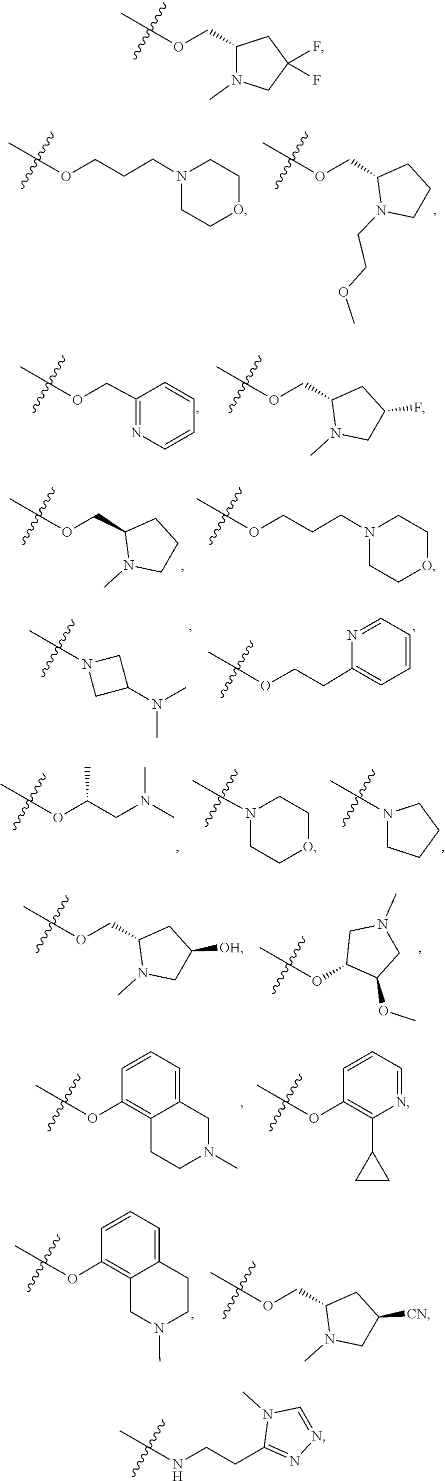

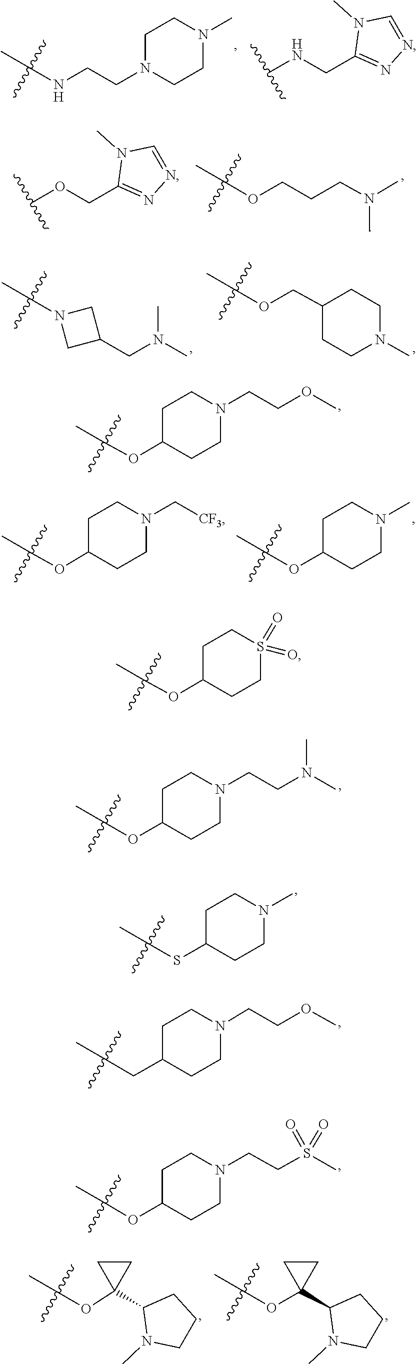

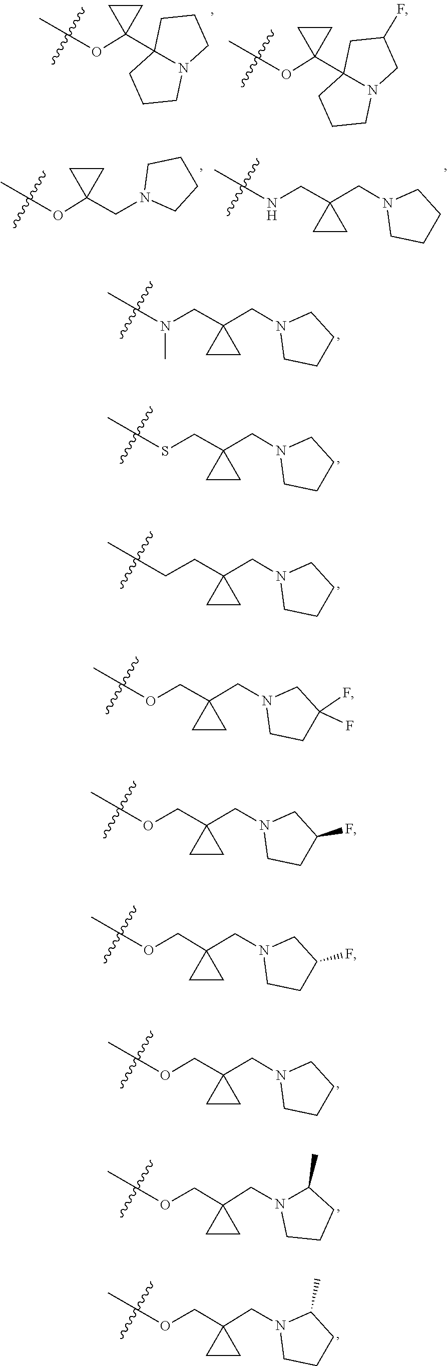

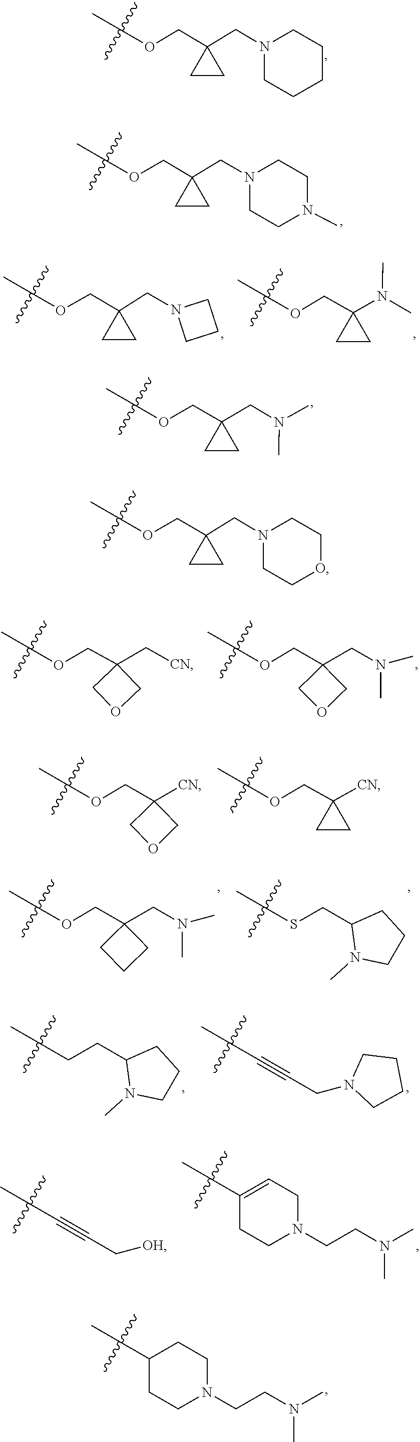

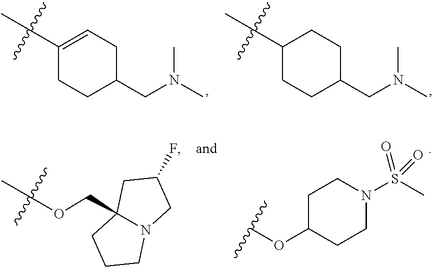 [SMILES: *C1=CCC(CN(C)C)CC1 ; *C1CCC(CN(C)C)CC1 ; *OC[C@]12CCCN1C[C@@H](F)C2 ; *OC1CCN(S(C)(=O)=O)CC1]

Embodiment A129 The compound of any one of Embodiments A1-A128, or a pharmaceutically acceptable salt or solvate thereof, wherein L¹ is a bond.

Embodiment A130 The compound of any one of Embodiments A1-A128, or a pharmaceutically acceptable salt or solvate thereof, wherein L¹ is O.

Embodiment A131 The compound of any one of Embodiments A1-A130, or a pharmaceutically acceptable salt or solvate thereof, wherein R¹⁹ is C₁₋₉heteroaryl optionally substituted with one, two, or three R²⁰ⁱ.

Embodiment A132 The compound of any one of Embodiments A1-A130, or a pharmaceutically acceptable salt or solvate thereof, wherein R¹⁹ is C₆₋₁₀aryl optionally substituted with one, two, or three R²⁰ⁱ.

Embodiment A133 The compound of any one of Embodiments A1-A132, or a pharmaceutically acceptable salt or solvate thereof, wherein L² is selected from a bond, C₁-C₆alkyl, and —C(O)—.

Embodiment A134 The compound of any one of Embodiments A1-A133, or a pharmaceutically acceptable salt or solvate thereof, wherein L² is a bond.

Embodiment A135 The compound of any one of Embodiments A1-A133, or a pharmaceutically acceptable salt or solvate thereof, wherein L² is a C₁-C₆alkyl.

Embodiment A136 The compound of any one of Embodiments A1-A135, or a pharmaceutically acceptable salt or solvate thereof, wherein R⁵ is an electrophilic moiety capable of forming a covalent bond with the cysteine residue at position 12 of a KRAS protein.

Embodiment A137 The compound of Embodiment A136, or a pharmaceutically acceptable salt or solvate thereof, wherein R⁵ is selected from the group consisting of

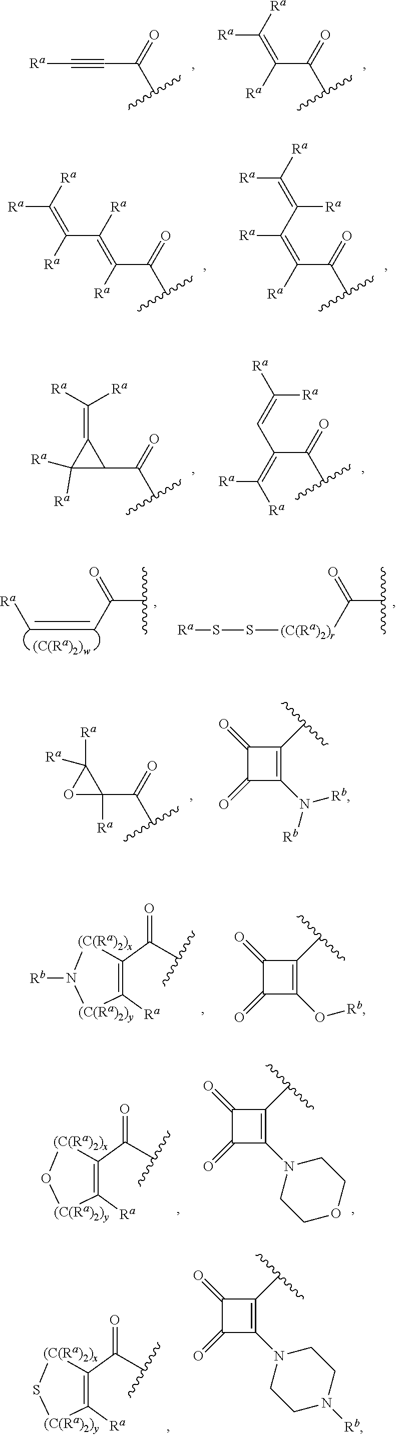

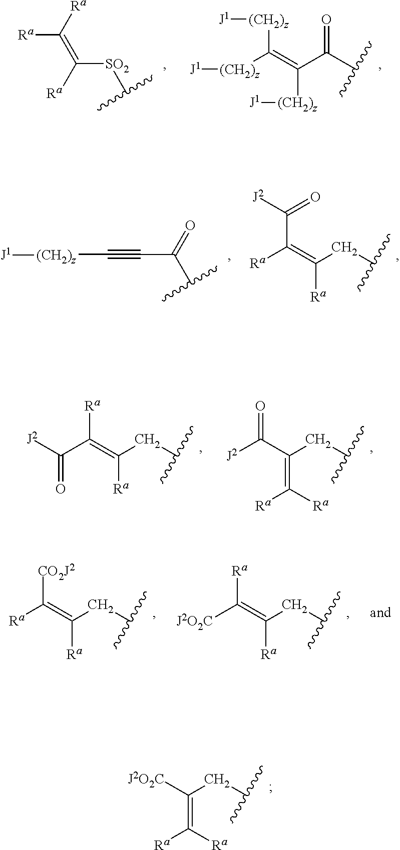

where each R^{a} is independently hydrogen, C₁₋₆alkyl, carboxy, C₁₋₆carboalkoxy, phenyl, C₂₋₇carboalkyl, R^{c}—(C(R^{b})₂)_{z}—, R^{c}—(C(R^{b})₂)_{w}-M-) (C(R^{b})₂), (R^{a})(R⁴)CH-M-(C(R^{b})₂)ᵣ, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; R^{c} is —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; M is —N(R^{b})—, —O—, —N[(C(R^{b})₂)_{w}—NR^{b}R^{b}]—, or —N[(C(R^{b})₂)_{w}—OR^{b}]—; J³ is —N(R^{b})—, —O—, or a bond; Het is a heterocycle, optionally mono- or di-substituted on carbon or nitrogen with Ro and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; r is 1-4; w is 2-4; x is 0-1; y is 0-4, and z is 1-6; wherein the sum of x+y is 2-4.

Embodiment A138 The compound of any one of Embodiments A1-A137, or a pharmaceutically acceptable salt or solvate thereof, wherein R⁵ is selected from the group consisting of:

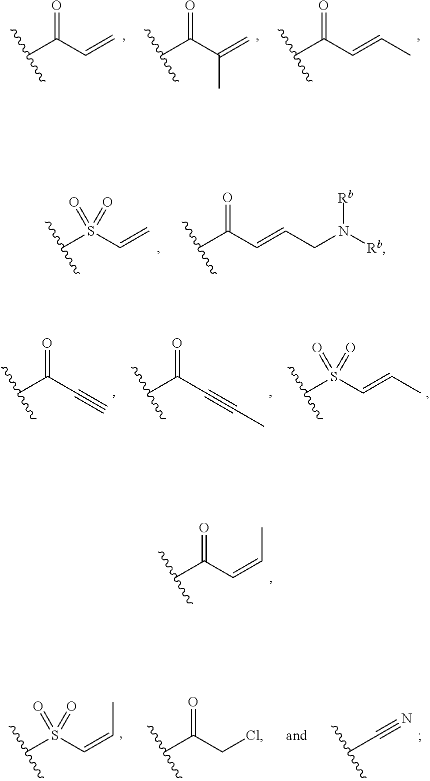

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

Embodiment B1 A compound of Formula (I), or a pharmaceutically acceptable salt or solvate thereof:

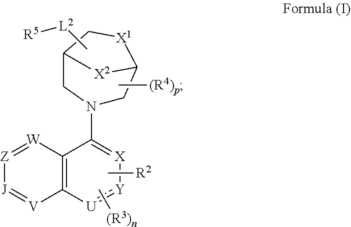

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(—NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from C(O), C═N—OR⁴, C—NNR⁴R⁶, N(R¹), N(R⁶), S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N;
Z is N or C(R⁸), wherein at least one of U, X, and Z is not N;
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—; R¹ is selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

Embodiment B2 The compound of Embodiment B1, or a pharmaceutically acceptable salt or solvate thereof, having the structure of Formula (Ia):

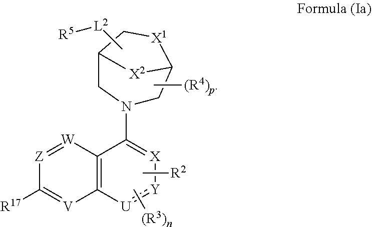

Embodiment B3 The compound of Embodiment B1 or Embodiment B2, or a pharmaceutically acceptable salt or solvate thereof, wherein Y is C.

Embodiment B4 The compound of Embodiment B1 or Embodiment B2, or a pharmaceutically acceptable salt or solvate thereof, wherein Y is N.

Embodiment B5 The compound of any one Embodiment B1 or Embodiment B2, or a pharmaceutically acceptable salt or solvate thereof, wherein Y is C(O).

Embodiment B6 The compound of any one of Embodiments B1-B5, or a pharmaceutically acceptable salt or solvate thereof, wherein X is C.

Embodiment B7 The compound of any one of Embodiments B1-B5, or a pharmaceutically acceptable salt or solvate thereof, wherein X is N.

Embodiment B8 The compound of any one of Embodiments B1-B7, or a pharmaceutically acceptable salt or solvate thereof, wherein U is C.

Embodiment B9 The compound of any one of Embodiments B1-B7, or a pharmaceutically acceptable salt or solvate thereof, wherein U is N.

Embodiment B10 The compound of any one of Embodiments B1-B7, or a pharmaceutically acceptable salt or solvate thereof, wherein U is C(O).

Embodiment B11 The compound of Embodiment B1 or Embodiment B2, or a pharmaceutically acceptable salt or solvate thereof, having the structure of Formula (Ib):

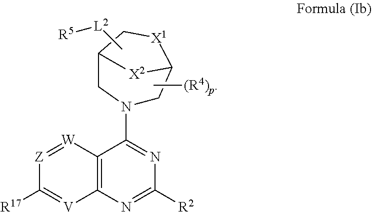

Embodiment B12 The compound of Embodiment B1 or Embodiment B2, or a pharmaceutically acceptable salt or solvate thereof, having the structure of Formula (Ic):

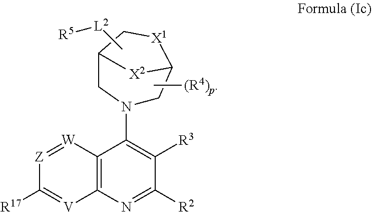

Embodiment B13 The compound of Embodiment B1 or Embodiment B2, or a pharmaceutically acceptable salt or solvate thereof, having the structure of Formula (Id):

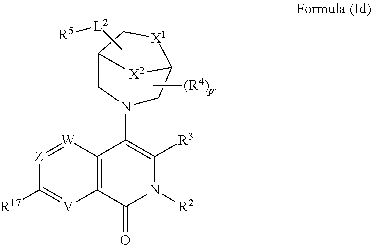

Embodiment B14 The compound of Embodiment B1 or Embodiment B2, or a pharmaceutically acceptable salt or solvate thereof, having the structure of Formula (Ie):

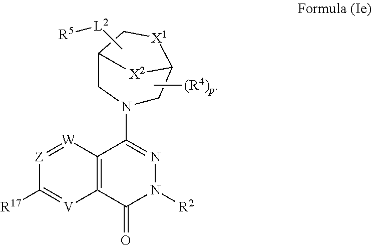

Embodiment B15 The compound of Embodiment B1 or Embodiment B2, or a pharmaceutically acceptable salt or solvate thereof, having the structure of Formula (If):

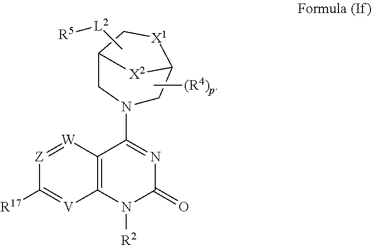

Embodiment B161 The compound of any one of Embodiments B1-B15, or a pharmaceutically acceptable salt or solvate thereof, wherein X² is selected from X³ and —CH₂—.

Embodiment B7 The compound of any one of Embodiments B1-B16, or a pharmaceutically acceptable salt or solvate thereof, wherein X² is X³.

Embodiment B18 The compound of any one of Embodiments B1-B17, or a pharmaceutically acceptable salt or solvate thereof, wherein X³ is N(R¹).

Embodiment B19 The compound of Embodiment B18, or a pharmaceutically acceptable salt or solvate thereof, wherein R¹ is selected from hydrogen and C₁₋₆alkyl.

Embodiment B20 The compound of Embodiment B19, or a pharmaceutically acceptable salt or solvate thereof, wherein R¹ is hydrogen.

Embodiment B21 The compound of Embodiment B19, or a pharmaceutically acceptable salt or solvate thereof, wherein R¹ is C₁₋₆alkyl.

Embodiment B22 A compound of Formula (II), or a pharmaceutically acceptable salt or solvate thereof:

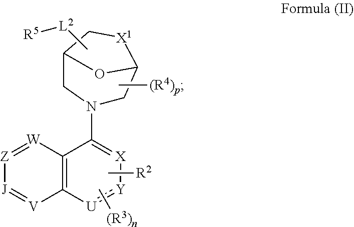

wherein:
X is C or N;
X¹ is selected from C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), and S(O)₂;
Y is C, S(O), S(O)₂, C(O), or N;
U is C, S(O), S(O)₂, C(O), or N, wherein two of U, Y, and X are N;
Z is N or C(R⁸);
V and J are independently selected from N, C(R¹⁶), and C(R¹⁷), wherein one of V and J is C(R¹⁷);
W is N or C(R¹⁸);
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
each R⁵ is independently hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, naphthalenyl, and C₁₋₉heteroaryl, wherein C₃₋₆cycloalkyl, C₂₋₉heterocycloalkyl, naphthalenyl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently 8selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 0, 1, 2, 3, 4, or 5; and
[symbol] indicates a single or double bond such that all valences are satisfied.

Embodiment B23 The compound of Embodiment B22, or a pharmaceutically acceptable salt or solvate thereof, having the structure of Formula (IIa):

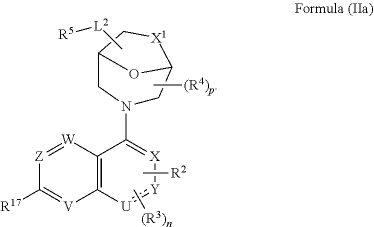

Embodiment B242 The compound of Embodiment B22 or Embodiment B23, or a pharmaceutically acceptable salt or solvate thereof, having the structure of Formula (IIb):

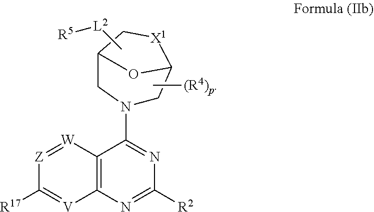

Embodiment B25 The compound of Embodiment B22 or Embodiment B23, or a pharmaceutically acceptable salt or solvate thereof, having the structure of Formula (IIc):

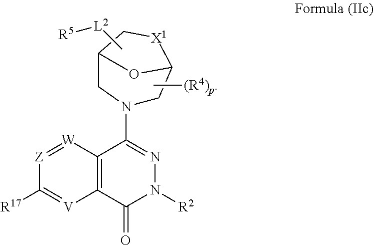

Embodiment B26 The compound of Embodiment B22 or Embodiment B23, or a pharmaceutically acceptable salt or solvate thereof, having the structure of Formula (IId):

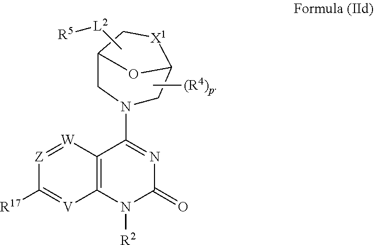

Embodiment B27 The compound of any one of Embodiments B1-B26, or a pharmaceutically acceptable salt or solvate thereof, wherein Z is C(R⁸).

Embodiment B28 The compound of any one of Embodiments B1-B27, or a pharmaceutically acceptable salt or solvate thereof, wherein R⁸ is hydrogen or halogen.

Embodiment B29 The compound of any one of Embodiments B1-B26, or a pharmaceutically acceptable salt or solvate thereof, wherein Z is N.

Embodiment B30 The compound of any one of Embodiments B1-B29, or a pharmaceutically acceptable salt or solvate thereof, wherein V is C(R¹⁶).

Embodiment B31 The compound of any one of Embodiments B1-B30, or a pharmaceutically acceptable salt or solvate thereof, wherein R¹⁶ is hydrogen or halogen.

Embodiment B32 The compound of any one of Embodiments B1-B29, or a pharmaceutically acceptable salt or solvate thereof, wherein V is N.

Embodiment B33 The compound of any one of Embodiments B1-B32, or a pharmaceutically acceptable salt or solvate thereof, wherein W is C(R¹⁸).

Embodiment B34 The compound of any one of Embodiments B1-B33, or a pharmaceutically acceptable salt or solvate thereof, wherein R¹⁸ is hydrogen.

Embodiment B35 The compound of any one of Embodiments B1-B32, or a pharmaceutically acceptable salt or solvate thereof, wherein W is N.

Embodiment B36. The compound of any one of Embodiments B1-B35, or a pharmaceutically acceptable salt or solvate thereof, wherein X¹ is N(R⁴).

Embodiment B370The compound of any one of Embodiments B1-B36, or a pharmaceutically acceptable salt or solvate thereof, wherein X¹ is N(H).

Embodiment B38 The compound of any one of Embodiments B1-B35, or a pharmaceutically acceptable salt or solvate thereof, wherein X¹ is N(R⁶).

Embodiment B39 The compound of any one of Embodiments B1-B35, or a pharmaceutically acceptable salt or solvate thereof, wherein X¹ is O.

Embodiment B40 The compound of any one of Embodiments B1-B35, or a pharmaceutically acceptable salt or solvate thereof, wherein X¹ is S.

Embodiment B41 The compound of any one of Embodiments B1-B35, or a pharmaceutically acceptable salt or solvate thereof, wherein X¹ is S(O)₂.

Embodiment B42 The compound of any one of Embodiments B1-B35, or a pharmaceutically acceptable salt or solvate thereof, wherein X¹ is C(R⁴)(R⁶).

Embodiment B43 The compound of any one of Embodiments B1-B35, or a pharmaceutically acceptable salt or solvate thereof, wherein X¹ is C(H)(R⁴).

Embodiment B44 The compound of any one of Embodiments B1-B35, or a pharmaceutically acceptable salt or solvate thereof, wherein X¹ is CH₂.

Embodiment B45 The compound of any one of Embodiments B1-B35, or a pharmaceutically acceptable salt or solvate thereof, wherein X¹ is C(H)(-L²-R⁵).

Embodiment B46 The compound of any one of Embodiments B1-B45, or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}.

Embodiment B47 The compound of any one of Embodiments B1-B46, or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}.

Embodiment B48 The compound of any one of Embodiments B1-B47, or a pharmaceutically acceptable salt or solvate thereof, wherein R² is —OR¹².

Embodiment B49 The compound of any one of Embodiments B1-B48, or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}.

Embodiment B50 The compound of any one of Embodiments B1-B48, or a pharmaceutically acceptable salt or solvate thereof, wherein R¹² is C₁₋₆alkyl optionally substituted with one, two, or three R^{20d}.

Embodiment B51 The compound of any one of Embodiments B1-B48, or a pharmaceutically acceptable salt or solvate thereof, wherein R¹² is C₂₋₉heterocycloalkyl optionally substituted with one, two, or three R^{20d}.

Embodiment B52 The compound of any one of Embodiments B1-B48, or a pharmaceutically acceptable salt or solvate thereof, wherein R¹² is —CH₂—C₂₋₉heterocycloalkyl optionally substituted with one, two, or three R^{20d}.

Embodiment B53 The compound of any one of Embodiments B1-B52, or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³).

Embodiment B54 The compound of any one of Embodiments B1-B53, or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

Embodiment B55 The compound of any one of Embodiments B1-B53, or a pharmaceutically acceptable salt or solvate thereof, wherein R² is selected from

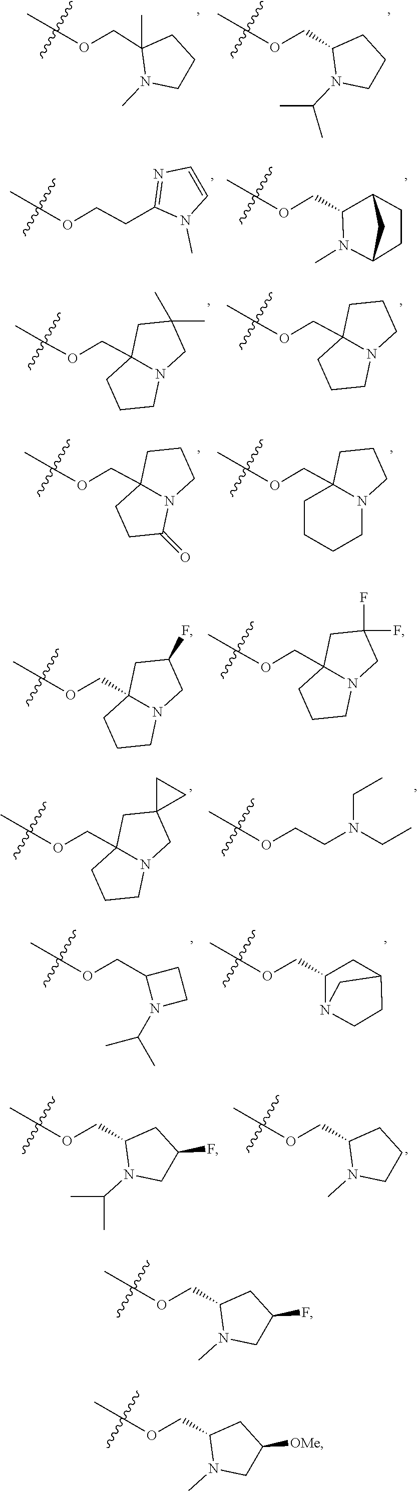

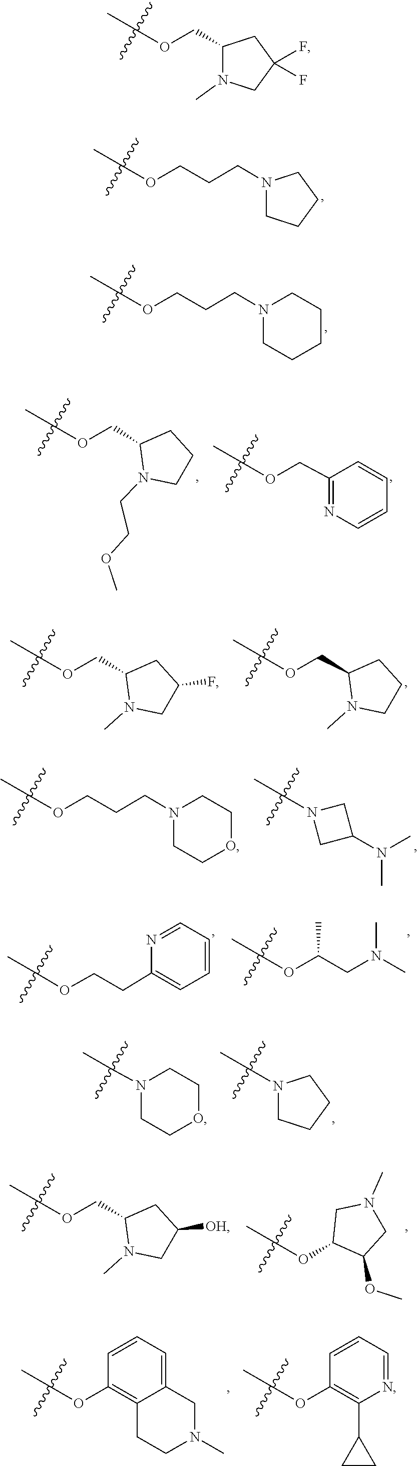

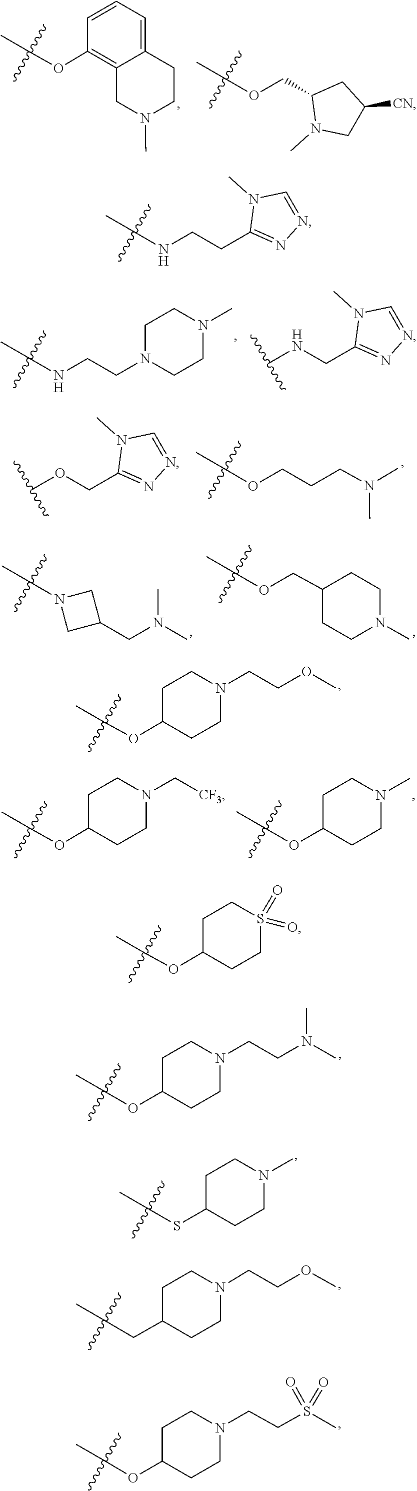

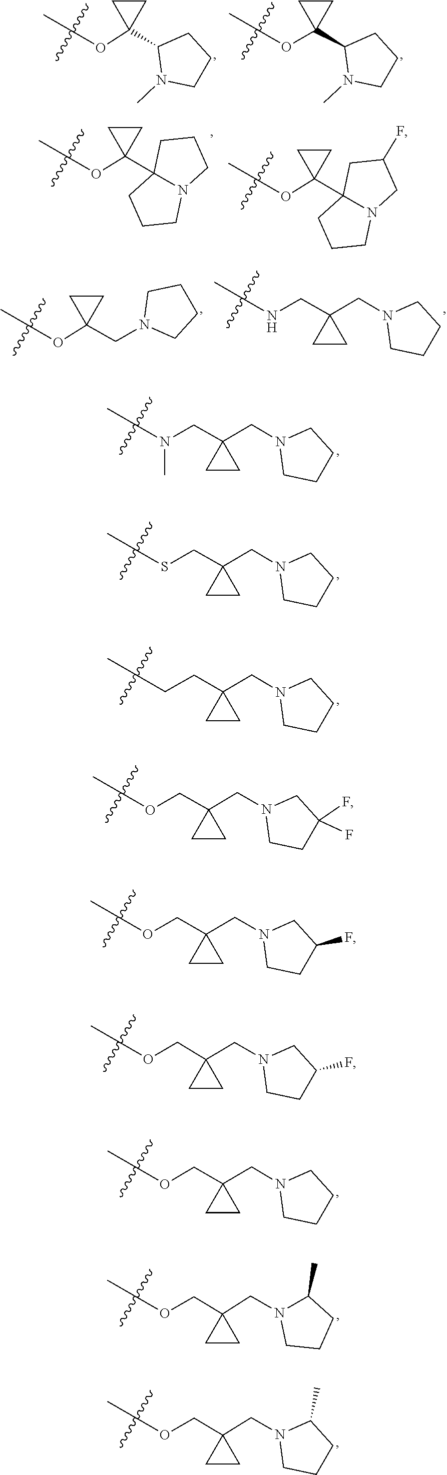

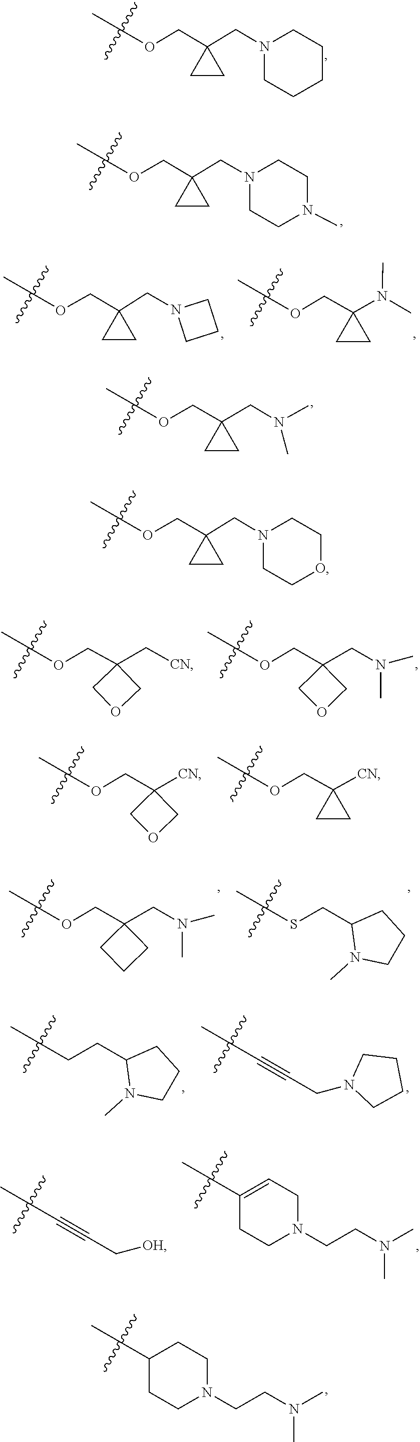

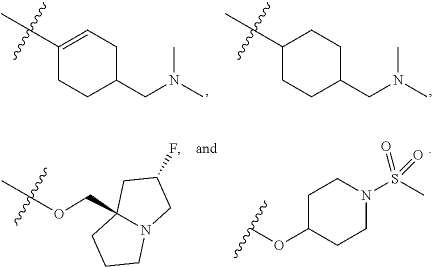 [SMILES: *C1=CCC(CN(C)C)CC1 ; *C1CCC(CN(C)C)CC1 ; *OC[C@]12CCCN1C[C@@H](F)C2 ; *OC1CCN(S(C)(=O)=O)CC1]

Embodiment B56 The compound of any one of Embodiments B1-B55, or a pharmaceutically acceptable salt or solvate thereof, wherein L¹ is a bond.

Embodiment B57 The compound of any one of Embodiments B1-B55, or a pharmaceutically acceptable salt or solvate thereof, wherein L¹ is O.

Embodiment B58 The compound of any one of Embodiments B1-B57, or a pharmaceutically acceptable salt or solvate thereof, wherein R¹⁹ is C₁₋₉heteroaryl optionally substituted with one, two, or three R²⁰ⁱ.

Embodiment B59 The compound of any one of Embodiments B1-B57, or a pharmaceutically acceptable salt or solvate thereof, wherein R¹⁹ is C₆₋₁₀aryl optionally substituted with one, two, or three R²⁰ⁱ.

Embodiment B60 The compound of any one of Embodiments B1-B59, or a pharmaceutically acceptable salt or solvate thereof, wherein p is 0.

Embodiment B61 The compound of any one of Embodiments B1-B60, or a pharmaceutically acceptable salt or solvate thereof, wherein each L² is selected from a bond, C₁-C₆alkyl, and —C(O)—.

Embodiment B62 The compound of any one of Embodiments B1-B61, or a pharmaceutically acceptable salt or solvate thereof, wherein each L² is a bond.

Embodiment B63 The compound of any one of Embodiments B1-B61, or a pharmaceutically acceptable salt or solvate thereof, wherein each L² is a C₁-C₆alkyl.

Embodiment B64 The compound of any one of Embodiments B1-B62, or a pharmaceutically acceptable salt or solvate thereof, wherein each R⁵ is hydrogen.

Embodiment B65 The compound of any one of Embodiments B1-B62, or a pharmaceutically acceptable salt or solvate thereof, wherein each R⁵ is a group other than an electrophilic moiety capable of forming a covalent bond with the cysteine residue at position 12 of a KRAS protein.

Embodiment B66 The compound of Embodiment B65, or a pharmaceutically acceptable salt or solvate thereof, wherein each R⁵ is selected from: —H, —NH₂, —OH, —NH(C₁₋₆ alkyl),

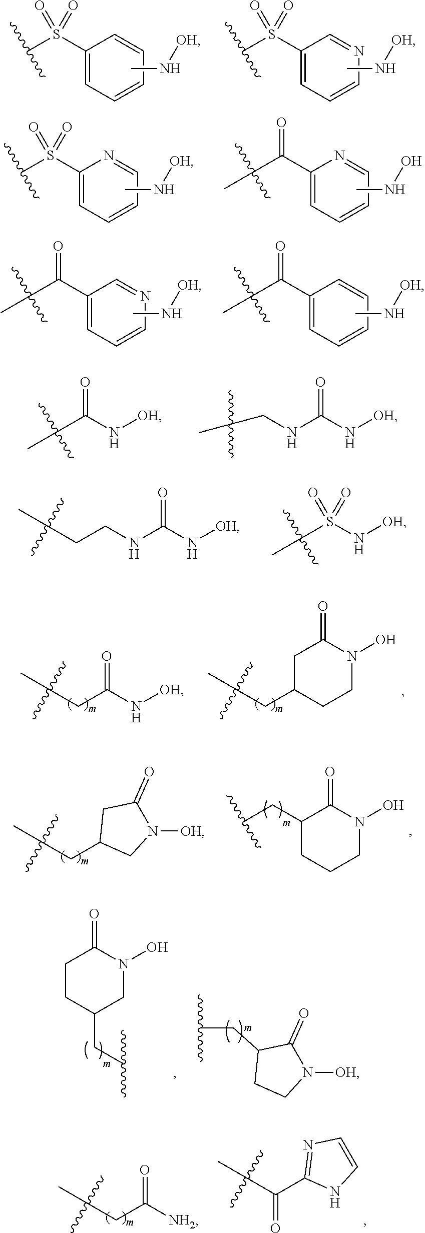

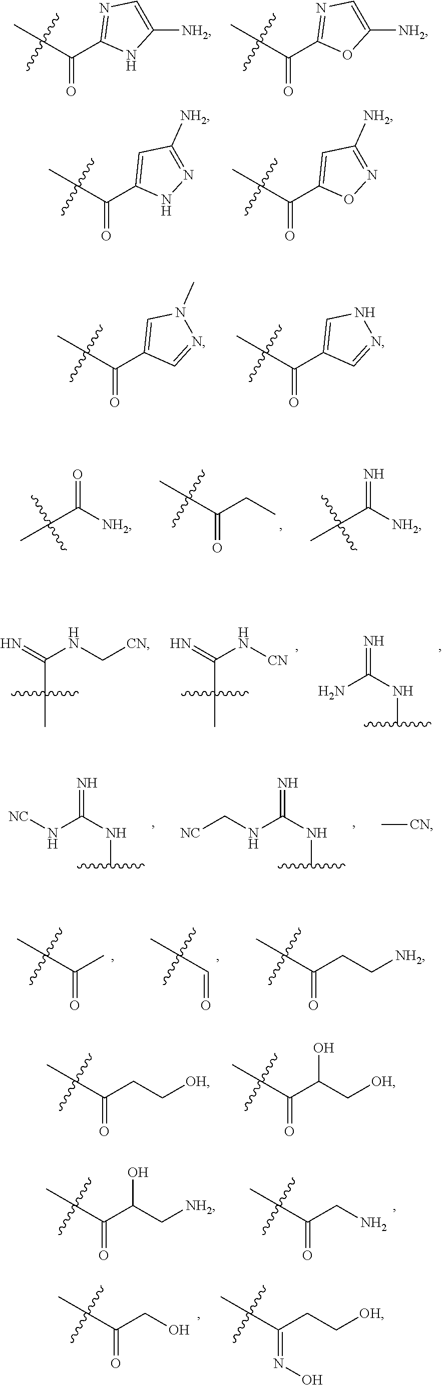

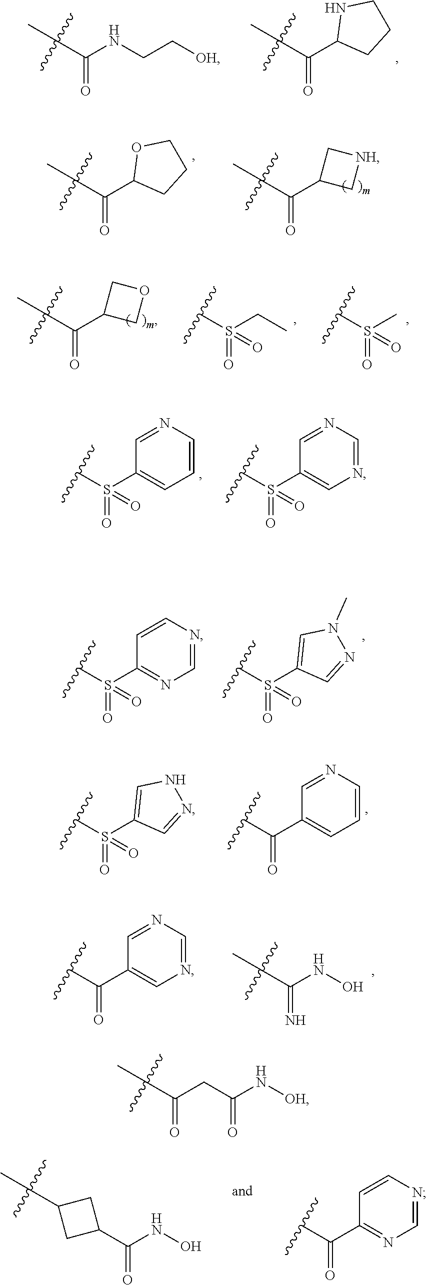

and m, when present, is 0, 1, 2, or 3.

Embodiment B67 A compound selected from:

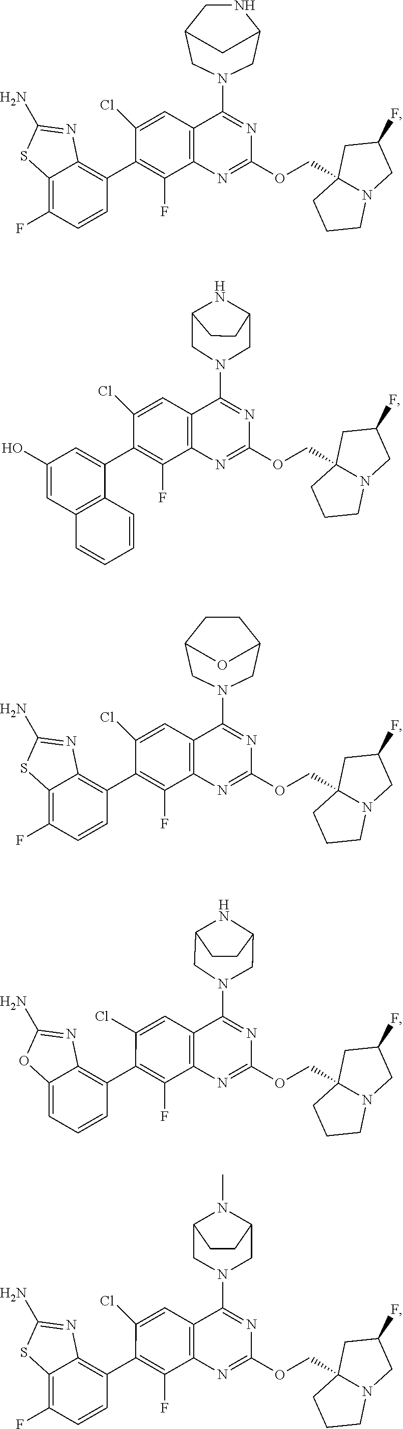 [SMILES: Nc1nc2c(-c3c(Cl)cc4c(N5CC6CNC(C6)C5)nc(OC[C@@]56CCCN5C[C@H](F)C6)nc4c3F)ccc(F)c2s1 ; Oc1cc(-c2c(Cl)cc3c(N4CC5CCC(C4)N5)nc(OC[C@@]45CCCN4C[C@H](F)C5)nc3c2F)c2ccccc2c1 ; Nc1nc2c(-c3c(Cl)cc4c(N5CC6CCC(C5)O6)nc(OC[C@@]56CCCN5C[C@H](F)C6)nc4c3F)ccc(F)c2s1 ; Nc1nc2c(-c3c(Cl)cc4c(N5CC6CCC(C5)N6)nc(OC[C@@]56CCCN5C[C@H](F)C6)nc4c3F)cccc2o1 ; CN1C2CCC1CN(c1nc(OC[C@@]34CCCN3C[C@H](F)C4)nc3c(F)c(-c4ccc(F)c5sc(N)nc45)c(Cl)cc13)C2]

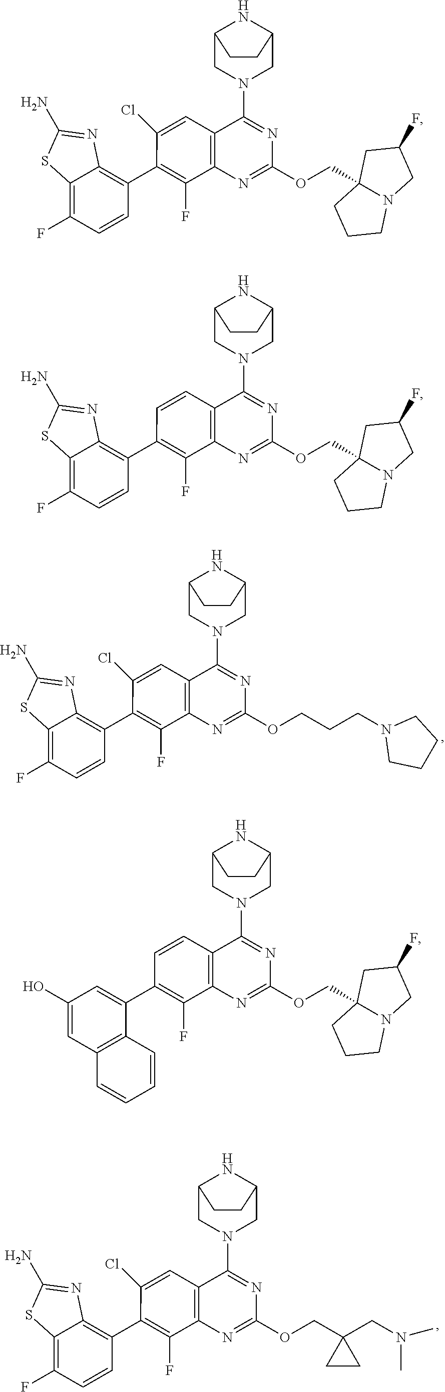 [SMILES: Nc1nc2c(-c3c(Cl)cc4c(N5CC6CCC(C5)N6)nc(OC[C@@]56CCCN5C[C@H](F)C6)nc4c3F)ccc(F)c2s1 ; Nc1nc2c(-c3ccc4c(N5CC6CCC(C5)N6)nc(OC[C@@]56CCCN5C[C@H](F)C6)nc4c3F)ccc(F)c2s1 ; Nc1nc2c(-c3c(Cl)cc4c(N5CC6CCC(C5)N6)nc(OCCCN5CCCC5)nc4c3F)ccc(F)c2s1 ; Oc1cc(-c2ccc3c(N4CC5CCC(C4)N5)nc(OC[C@@]45CCCN4C[C@H](F)C5)nc3c2F)c2ccccc2c1 ; CN(C)CC1(COc2nc(N3CC4CCC(C3)N4)c3cc(Cl)c(-c4ccc(F)c5sc(N)nc45)c(F)c3n2)CC1]

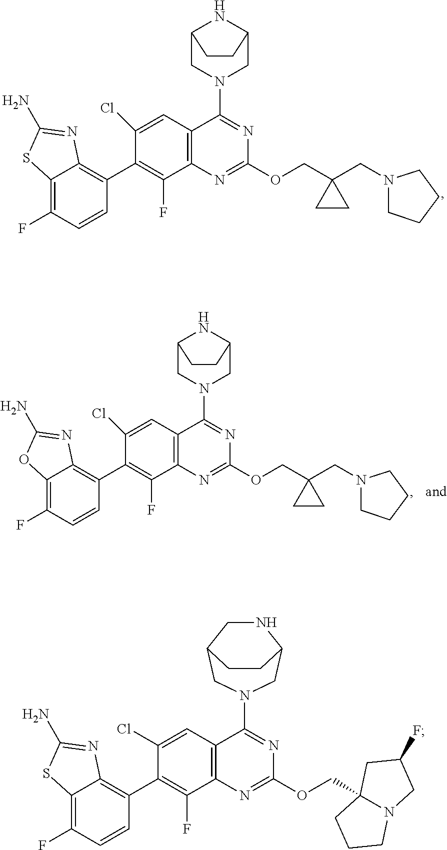 [SMILES: Nc1nc2c(-c3c(Cl)cc4c(N5CC6CCC(C5)N6)nc(OCC5(CN6CCCC6)CC5)nc4c3F)ccc(F)c2s1 ; Nc1nc2c(-c3c(Cl)cc4c(N5CC6CCC(C5)N6)nc(OCC5(CN6CCCC6)CC5)nc4c3F)ccc(F)c2o1 ; Nc1nc2c(-c3c(Cl)cc4c(N5CC6CCC(C5)NC6)nc(OC[C@@]56CCCN5C[C@H](F)C6)nc4c3F)ccc(F)c2s1]

or a pharmaceutically acceptable salt or solvate thereof.

Embodiment C1 A compound of Formula (I), or a pharmaceutically acceptable salt or solvate thereof:

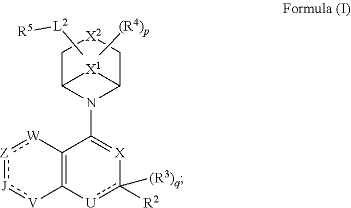

wherein:
X is C(R³) or N;
X¹ is selected from CH₂, C(R⁴)(R⁶), C—N—OR⁴, C═NN(R⁴)(R⁶), C(O)N(R⁴), N(R⁴), N(R⁶), O, S, S(O), S(—O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C—N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁴)OC(O)N(R⁴), C(R⁴)(R⁶)N(R⁴)C(O)N(R⁴), C(R⁴)(R⁶)S(O)₂, C—NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C—N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═OX(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X² is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R¹⁸)O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h}; R² is -L³-R^{12b};
L³ is selected from C₁-C₆alkyl, C₂₋₆alkenyl, C₂₋₆alkynyl, —O—, —N(R¹⁴)—, and —S—;
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C_{1- 9}heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

Embodiment C2 The compound of embodiment C1, or a pharmaceutically acceptable salt or solvate thereof, wherein X is C(R³).

Embodiment C3 The compound of embodiment C1, or a pharmaceutically acceptable salt or solvate thereof, wherein X is N.

Embodiment C4 The compound of any one of embodiments C1-C3, or a pharmaceutically acceptable salt or solvate thereof, wherein U is C(R³).

Embodiment C5 The compound of any one of embodiments C1-C3, or a pharmaceutically acceptable salt or solvate thereof, wherein U is N(R³).

Embodiment C6 The compound of any one of embodiments C1-C3, or a pharmaceutically acceptable salt or solvate thereof, wherein U is N.

Embodiment C7 The compound of any one of embodiments C1-C3, or a pharmaceutically acceptable salt or solvate thereof, wherein U is C(O).

Embodiment C8 The compound of embodiment C1, or a pharmaceutically acceptable salt or solvate thereof, having the structure of Formula (Ia):

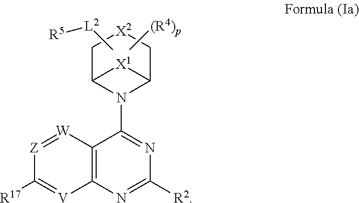

Embodiment C9 The compound of embodiment C1, or a pharmaceutically acceptable salt or solvate thereof, having the structure of Formula (Ib):

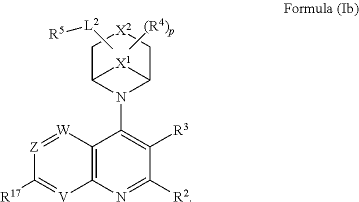

Embodiment C10 The compound of any one of embodiments C1-C9, or a pharmaceutically acceptable salt or solvate thereof, wherein Z is C(R⁸).

Embodiment C11 The compound of any one of embodiments C1-C10, or a pharmaceutically acceptable salt or solvate thereof, wherein R⁸ is hydrogen or halogen.

Embodiment C12 The compound of any one of embodiments C1-C9, or a pharmaceutically acceptable salt or solvate thereof, wherein Z is N.

Embodiment C13 The compound of any one of embodiments C1-C12, or a pharmaceutically acceptable salt or solvate thereof, wherein V is C(R¹⁶).

Embodiment C141 The compound of any one of embodiments C1-C13, or a pharmaceutically acceptable salt or solvate thereof, wherein R¹⁶ is hydrogen or halogen.

Embodiment C15 The compound of any one of embodiments C1-C12, or a pharmaceutically acceptable salt or solvate thereof, wherein V is N.

Embodiment C16 The compound of any one of embodiments C1-C15, or a pharmaceutically acceptable salt or solvate thereof, wherein W is C(R¹⁸).

Embodiment C17 The compound of any one of embodiments C1-C15, or a pharmaceutically acceptable salt or solvate thereof, wherein W is N(R¹⁸).

Embodiment C18 The compound of any one of embodiments C1-C17, or a pharmaceutically acceptable salt or solvate thereof, wherein R¹⁸ is hydrogen.

Embodiment C19 The compound of any one of embodiments C1-C15, or a pharmaceutically acceptable salt or solvate thereof, wherein W is N.

Embodiment C20 The compound of embodiment C1, or a pharmaceutically acceptable salt or solvate thereof, having the structure of Formula (Ic):

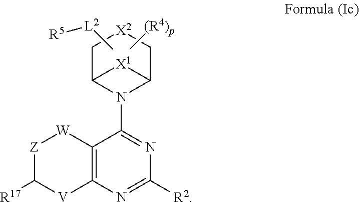

Embodiment C21 The compound of any one of embodiments C1-C7 and C20, or a pharmaceutically acceptable salt or solvate thereof, wherein Z is C(R⁸)(R^{8a}).

Embodiment C22 The compound of embodiment C21, or a pharmaceutically acceptable salt or solvate thereof, wherein R⁸ and R^{8a} are hydrogen.

Embodiment C23 The compound of any one of embodiments C1-C7 and C20, or a pharmaceutically acceptable salt or solvate thereof, wherein Z is N(R⁸).

Embodiment C24 The compound of any one of embodiments C1-C7 and C21-C23, or a pharmaceutically acceptable salt or solvate thereof, wherein V is C(R¹⁶)(R^{16a}).

Embodiment C25 The compound of embodiment C24, or a pharmaceutically acceptable salt or solvate thereof, wherein R¹⁶ and R^{16a} are hydrogen

Embodiment C26 The compound of any one of embodiments C1-C7 and C21-C23, or a pharmaceutically acceptable salt or solvate thereof, wherein V is N(R¹⁶).

Embodiment C27 The compound of any one of embodiments C1-C7 and C21-C26, or a pharmaceutically acceptable salt or solvate thereof, wherein W is C(R¹⁸)(R^{18a}).

Embodiment C28 The compound of embodiment C27, or a pharmaceutically acceptable salt or solvate thereof, wherein R¹⁸ and R^{18a} are hydrogen.

Embodiment C29 The compound of any one of embodiments C1-C7 and C21-C26, or a pharmaceutically acceptable salt or solvate thereof, wherein W is N(R¹⁸).

Embodiment C30 A compound of Formula (II), or a pharmaceutically acceptable salt or solvate thereof:

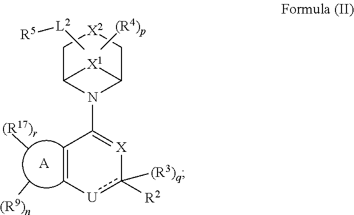

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁴)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X² is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a}; R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R¹, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20c}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

Embodiment C31 The compound of embodiment C30, or a pharmaceutically acceptable salt or solvate thereof, having the formula (IIa):

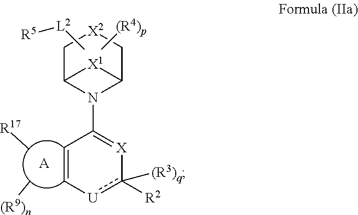

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁶)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X² is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a}; R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein; R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R^{1g})O, C(R¹⁵)(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl, wherein the bicyclic C₄₋₁₂cycloalkyl, bicyclic C₂₋₁₁heterocycloalkyl, naphthalenyl, fused ring C₇₋₁₂aryl, bicyclic C₂₋₁₂heteroaryl, and fused ring C₂₋₁₂heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
each R⁹ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, or 5;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁-haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

Embodiment C32 The compound of one of embodiments C30 to C31, or a pharmaceutically acceptable salt or solvate thereof, having the structure of Formula (IIc):

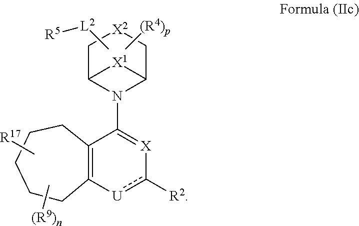

Embodiment C33 The compound of one of embodiments C30 to C31, or a pharmaceutically acceptable salt or solvate thereof, having the structure of Formula (IId):

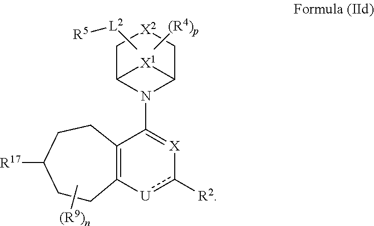

Embodiment C34 The compound of one of embodiments C30 to C31, or a pharmaceutically acceptable salt or solvate thereof, having the structure of Formula (IIe):

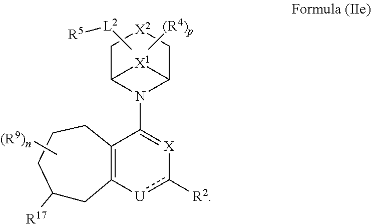

Embodiment C35 The compound of one of embodiments C30 to C31, or a pharmaceutically acceptable salt or solvate thereof, having the structure of Formula (IIf):

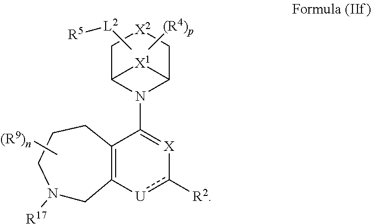

Embodiment C36 The compound of one of embodiments C30 to C31, or a pharmaceutically acceptable salt or solvate thereof, having the structure of Formula (IIg):

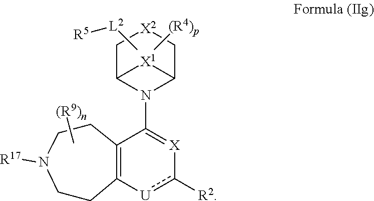

Embodiment C37 The compound of any one of embodiments C31 to C36, or a pharmaceutically acceptable salt or solvate thereof, wherein X is C(R³).

Embodiment C38 The compound of any one of embodiments C31 to C36, or a pharmaceutically acceptable salt or solvate thereof, wherein X is N.

Embodiment C39 The compound of any one of embodiments C31 to C38, or a pharmaceutically acceptable salt or solvate thereof, wherein U is C(R³).

Embodiment C40 The compound of any one of embodiments C31 to C38, or a pharmaceutically acceptable salt or solvate thereof, wherein U is N(R³).

Embodiment C41 The compound of any one of embodiments C31 to C38, or a pharmaceutically acceptable salt or solvate thereof, wherein U is N.

Embodiment C42 The compound of any one of embodiments C31 to C38, or a pharmaceutically acceptable salt or solvate thereof, wherein U is C(O).

Embodiment C43 The compound of any one of embodiments C31 to C42, or a pharmaceutically acceptable salt or solvate thereof, wherein each R⁹ is independently selected from halogen, C₁₋₆alkyl, C₃₋₆cycloalkyl, C₂₋₉heterocycloalkyl, C₆₋₁₀aryl, C₁₋₉heteroaryl, —OR¹², and —N(H)(R¹²), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20c}.

Embodiment C44 The compound of any one of embodiments C31 to C44, or a pharmaceutically acceptable salt or solvate thereof, wherein each R⁹ is independently selected from halogen, C₁₋₆alkyl, —OR¹², and —N(H)(R¹²), wherein C₁₋₆alkyl is optionally substituted with one, two, or three R^{20c}.

Embodiment C45 The compound of any one of embodiments C31 to C45, or a pharmaceutically acceptable salt or solvate thereof, wherein n is 1, 2, or 3.

Embodiment C46 The compound of any one of embodiments C31 to C42, or a pharmaceutically acceptable salt or solvate thereof, wherein n is 0.

Embodiment C47 The compound of any one of embodiments C1-C46, or a pharmaceutically acceptable salt or solvate thereof, wherein X¹ is CH₂C(R⁴)(R⁶).

Embodiment C48 The compound of any one of embodiments C1-C47, or a pharmaceutically acceptable salt or solvate thereof, wherein X¹ is —CH₂CH₂—.

Embodiment C49 The compound of any one of embodiments C1-C48, or a pharmaceutically acceptable salt or solvate thereof, wherein X² is N(R¹).

Embodiment C50 The compound of embodiment C49, or a pharmaceutically acceptable salt or solvate thereof, wherein X² is N(H).

Embodiment C51 The compound of any one of embodiments C1-C48, or a pharmaceutically acceptable salt or solvate thereof, wherein X² is O.

Embodiment C52 The compound of any one of embodiments C1-C48, or a pharmaceutically acceptable salt or solvate thereof, wherein X² is C(R⁴).

Embodiment C53 The compound of any one of embodiments C1-C48, or a pharmaceutically acceptable salt or solvate thereof, wherein X² is —CH₂—.

Embodiment C54 The compound of any one of embodiments C1-C53, or a pharmaceutically acceptable salt or solvate thereof, wherein L³ is selected from —O—, —N(R¹⁴)—, and —S—.

Embodiment C55 The compound of any one of embodiments C1-C54, or a pharmaceutically acceptable salt or solvate thereof, wherein L³ is —O—.

Embodiment C56 The compound of any one of embodiments C1-C55, or a pharmaceutically acceptable salt or solvate thereof, wherein R^{12b} is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or Embodiment C three R^{20d}.

Embodiment C57 The compound of any one of embodiments C1-C55, or a pharmaceutically acceptable salt or solvate thereof, wherein R^{12b} is C₁₋₆alkyl optionally substituted with one, two, or three R^{20d}.

Embodiment C58 The compound of any one of embodiments C1-C56, or a pharmaceutically acceptable salt or solvate thereof, wherein R^{12b} is C₂₋₉heterocycloalkyl optionally substituted with one, two, or three R^{20d}.

Embodiment C59 The compound of any one of embodiments C1-C56, or a pharmaceutically acceptable salt or solvate thereof, wherein R^{12b} is —CH₂—C₂₋₉heterocycloalkyl optionally substituted with one, two, or three R^{20d}.

Embodiment C60 The compound of any one of embodiments C1-C59, or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³).

Embodiment C61 The compound of any one of embodiments C1-C60, or a pharmaceutically acceptable salt or solvate thereof, wherein each R^{20d} is independently selected from halogen, C₁₋₆calkyl, and —OR²¹, wherein C₁₋₆calkyl is optionally substituted with one, two, or three groups independently selected from halogen, oxo, C₁₋₆alkyl, —OR²¹, and —N(R²²)(R²³).

Embodiment C62 The compound of any one of embodiments C1-C61, or a pharmaceutically acceptable salt or solvate thereof, wherein R³ is selected from

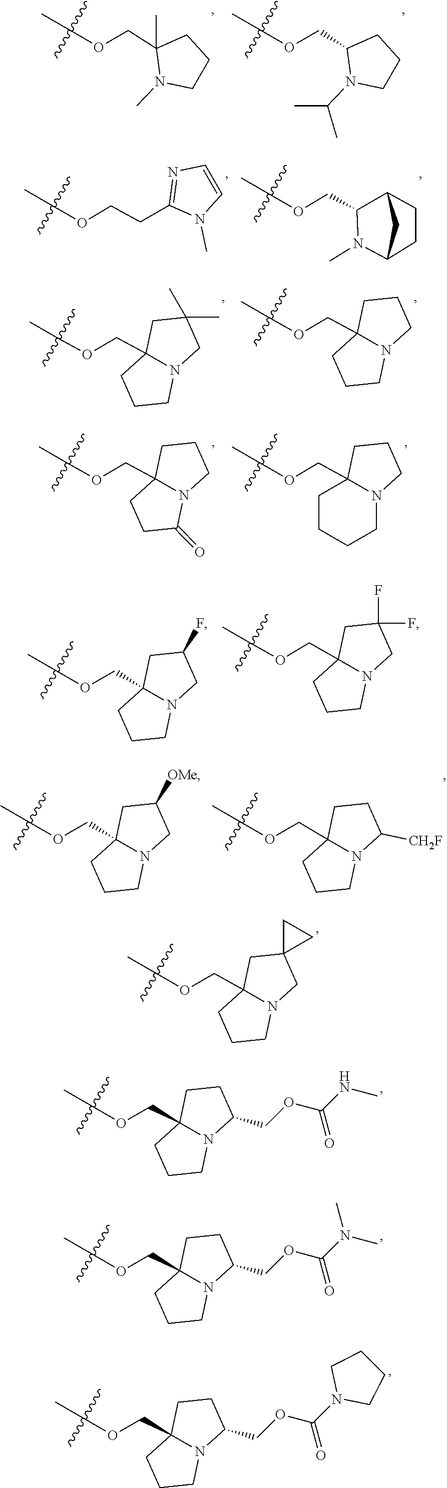

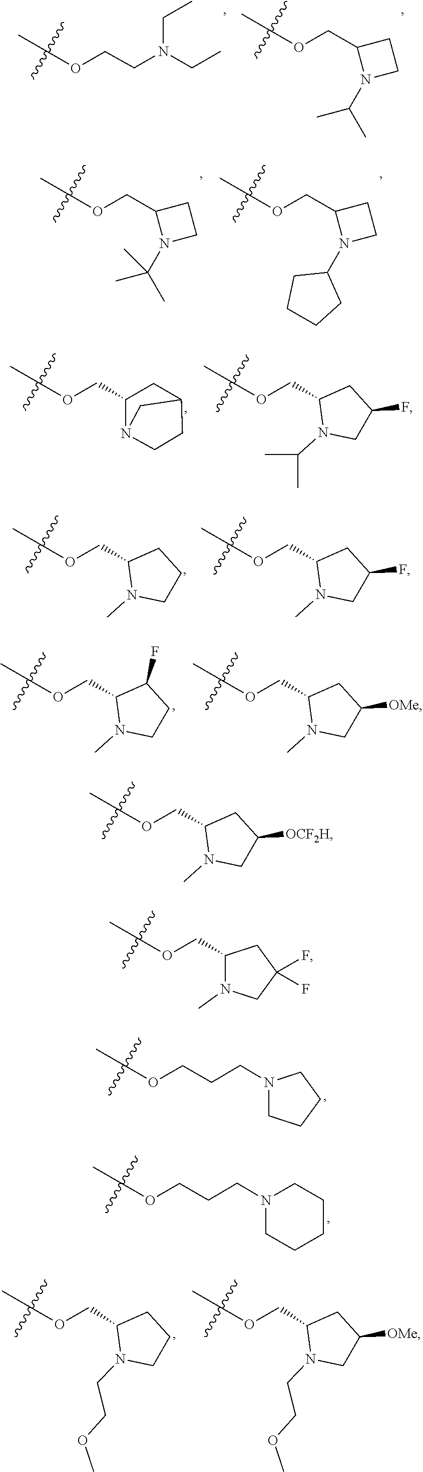

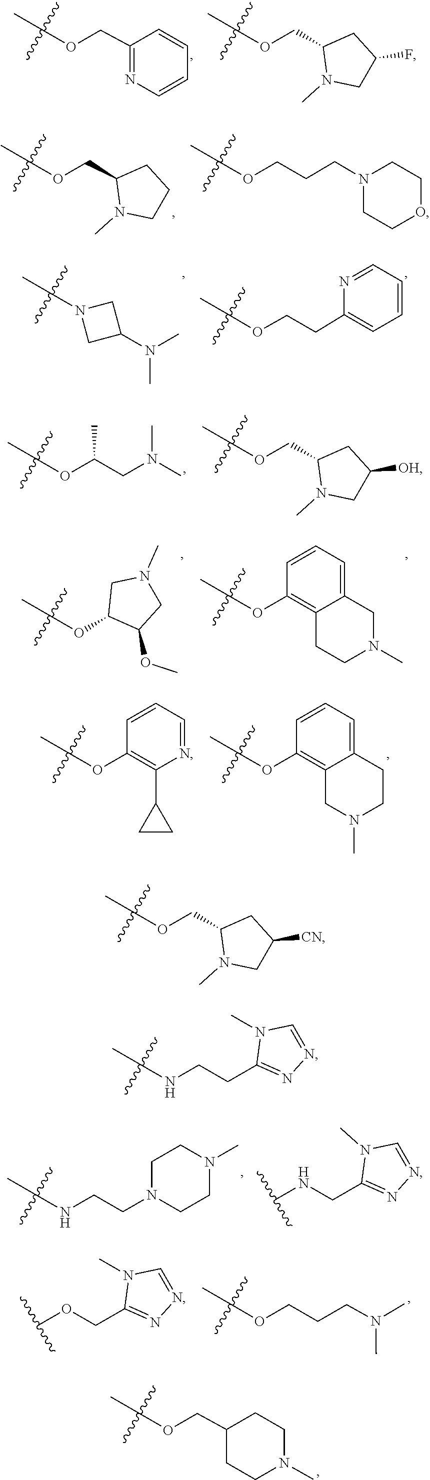

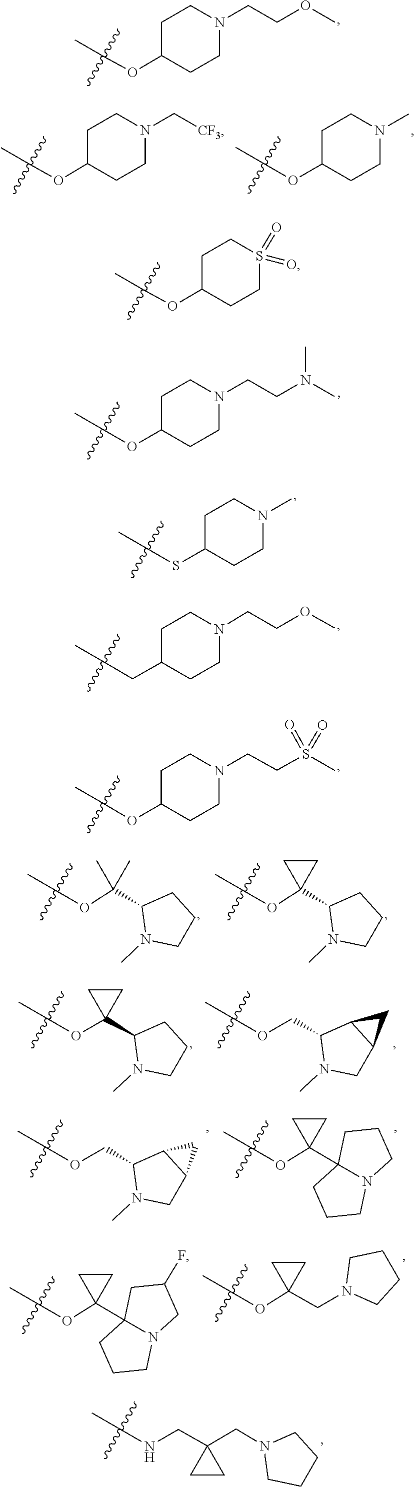

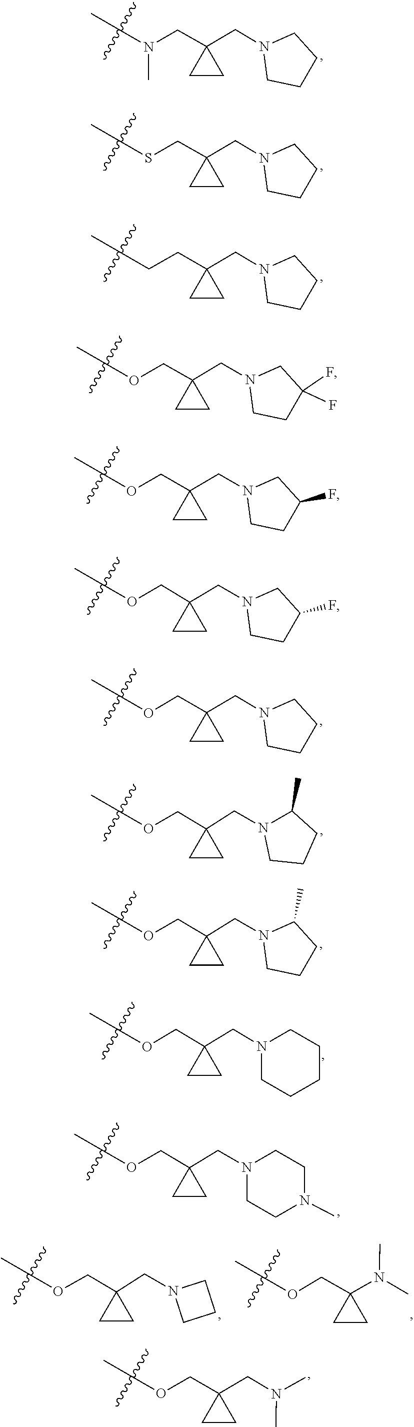

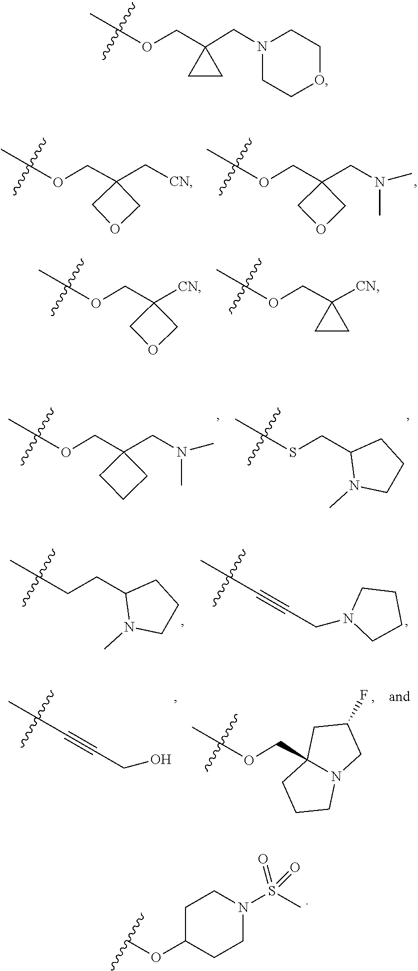

Embodiment C63 The compound of any one of embodiments C1-C62, or a pharmaceutically acceptable salt or solvate thereof, wherein L¹ is a bond.

Embodiment C64 The compound of any one of embodiments C1-C62, or a pharmaceutically acceptable salt or solvate thereof, wherein L¹ is O.

Embodiment C65 The compound of any one of embodiments C1-C64, or a pharmaceutically acceptable salt or solvate thereof, wherein R¹⁹ is a bicyclic C₂₋₁₂heteroaryl or a fused ring C₂₋₁₂heteroaryl, wherein the bicyclic C₂₋₁₂heteroaryl and fused ring C₂₋₁₂heteroaryl are optionally substituted with one, two, or three R²⁰ⁱ.

Embodiment C66 The compound of any one of embodiments C1-C65, or a pharmaceutically acceptable salt or solvate thereof, wherein R¹⁹ is selected from:

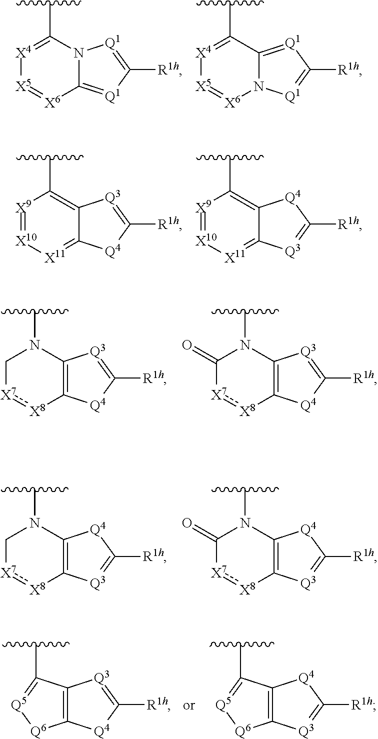

Q¹, Q³, and Q⁵ are independently N or C(R^{1d});
Q⁴ and Q⁶ are independently O, S, C(R^{1a})(R^{1b}), or N(R^{1c});
X⁴, X³, X⁶, X⁹, X¹⁰, and X¹¹ are independently selected from C(R^{1a}) or N;
X⁷ and X⁸ are independently selected from C(R^{1a}), C(R^{1a})(R^{1b}), N, or N(R^{1c});
each R^{1a}, R^{1b}, R^{1d}, R^{1f}, R¹⁸, and R^{1h} are each independently selected from hydrogen, halogen, —CN, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or Rif and R¹⁸ are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ, and
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

Embodiment C67 The compound of any one of embodiments C1-C66, or a pharmaceutically acceptable salt or solvate thereof, wherein R¹⁹ is selected from

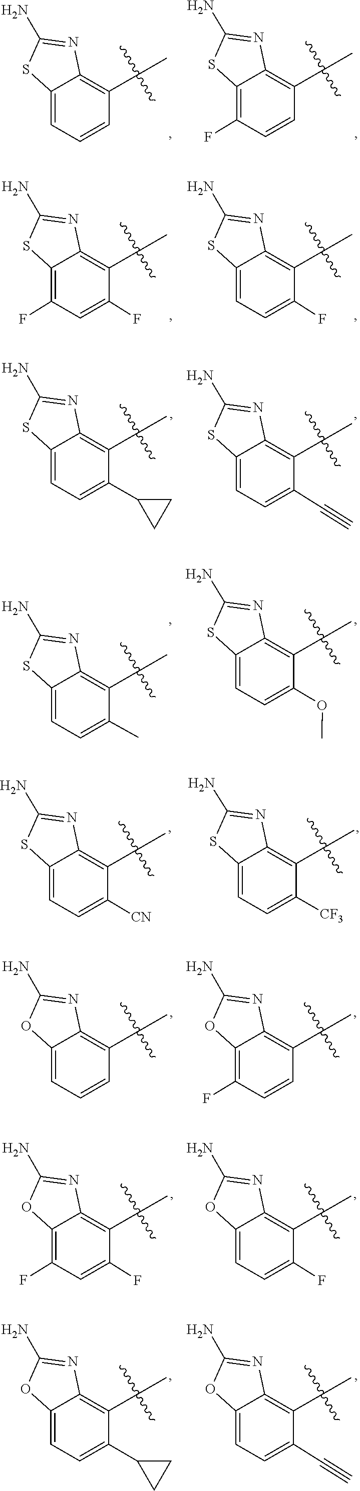

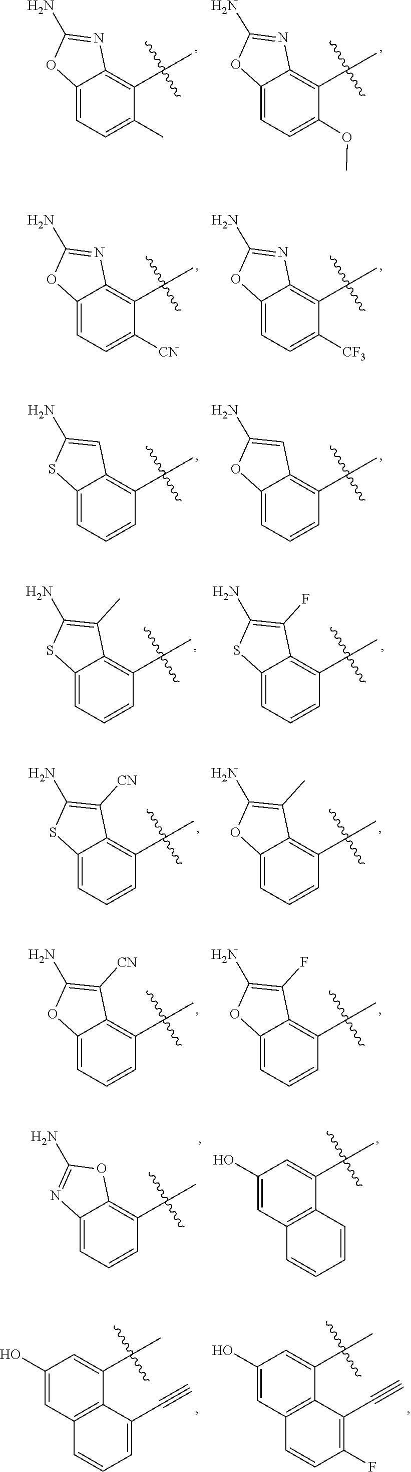

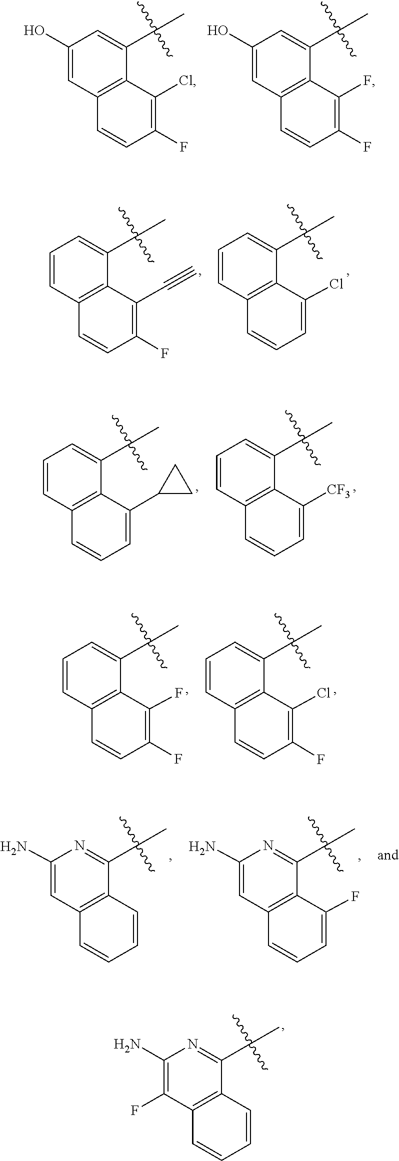

Embodiment C68 The compound of any one of embodiments C1-C67, or a pharmaceutically acceptable salt or solvate thereof, wherein p is 0.

Embodiment C69 The compound of any one of embodiments C1-C68, or a pharmaceutically acceptable salt or solvate thereof, wherein each L² is selected from a bond, C₁-C₆alkyl, and —C(O)—.

Embodiment C70 The compound of any one of embodiments C1-C69, or a pharmaceutically acceptable salt or solvate thereof, wherein each R⁵ is hydrogen.

Embodiment C71 The compound of any one of embodiments C1-C70, or a pharmaceutically acceptable salt or solvate thereof, wherein each R⁵ is a group other than an electrophilic moiety capable of forming a covalent bond with the cysteine residue at position 12 of a KRAS protein.

Embodiment C72 The compound of embodiment C71, or a pharmaceutically acceptable salt or solvate thereof, wherein each R⁵ is selected from: —H, —NH₂, —OH, —NH(C₁₋₆ alkyl),

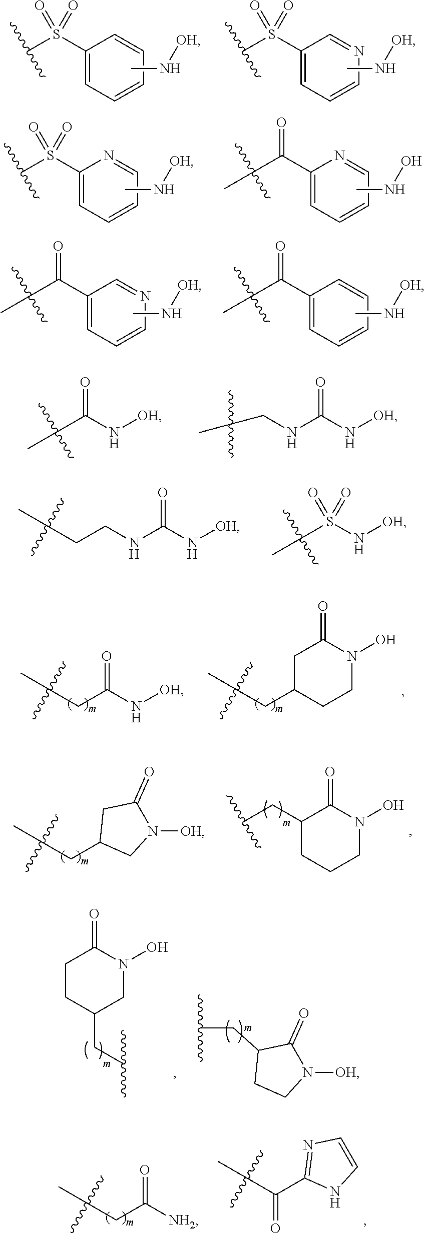

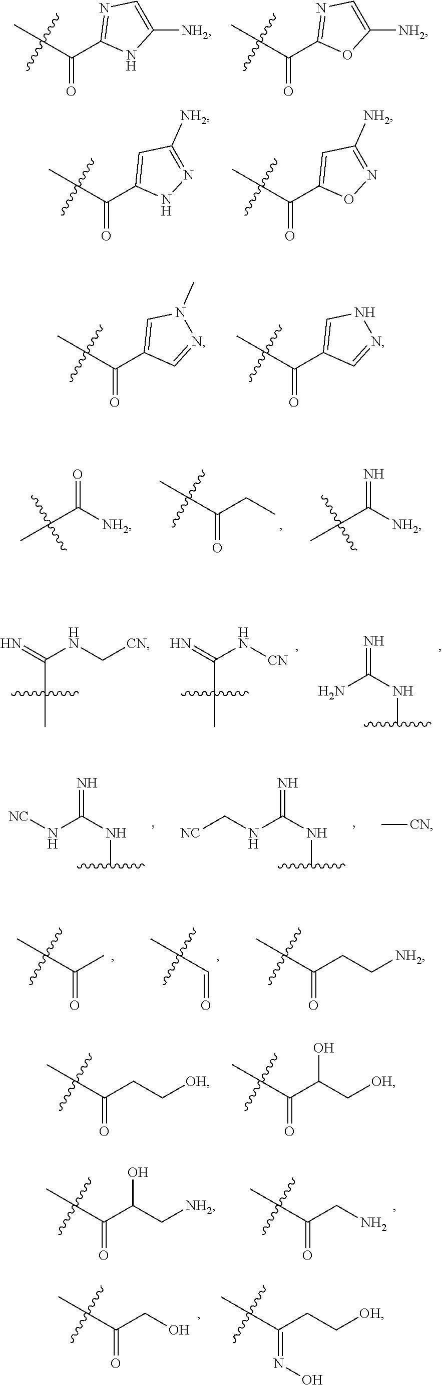

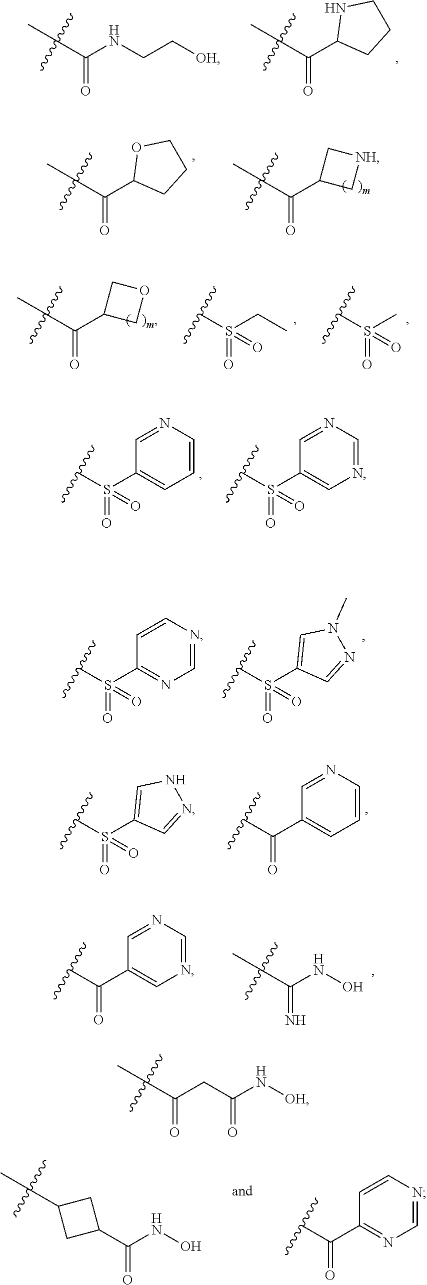

and m, when present, is 0, 1, 2, or 3.

Embodiment C73 A compound of Formula (III), or a pharmaceutically acceptable salt or solvate thereof:

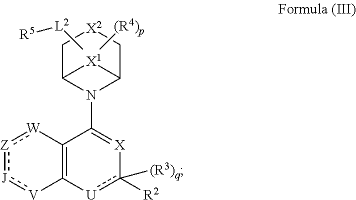

wherein:
X is C(R³) or N;
X¹ is selected from CH₂, C(R⁴)(R⁶), C—N—OR⁴, C═NN(R⁴)(R⁶), C(O)N(R⁴), N(R⁴), N(R⁶), O, S, S(O), S(—OX(—NR⁴), S(O), N(R⁴), N(R⁴)S(O)N(R⁴), N(R⁴)S(O)₂, N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁴)C(R⁴)(R⁴)C(R⁴)(R⁶), C(R⁴)(R⁴)C—N—OR⁴, CH₂C═NN(R⁴)(R⁶), C(R⁴)(R⁴)C(O)N(R⁴), C(R⁴)(RO)N(R⁴), C(R⁴)(R⁴)N(R⁶), C(R⁴)(R⁶)O), C(R⁴)(R⁴)OC(R⁴)(R⁴), C(R⁴)(R⁶)S, C(R⁴)(R⁴)SC(R⁴)(R⁴), C(R⁴)(R⁶)S(O), C(R⁴)(R⁶)S(O)C(R⁴)(R⁴), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X² is selected from N(R¹), N(R⁶), O, S, S(O), —C(H)(R⁶)—, and —C(R⁴)₂;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a};
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
q is 0 or 1;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
U is C(R³), C(R³)₂, S(O), S(O)₂, C(O), N(R³), or N;
Z is N, N(R⁸), C(R⁸), or C(R⁸)(R^{8a});
R⁸ and R^{8a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(—O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c}; J is selected from N, CH(R¹⁷), and C(R¹⁷);
R¹⁷ is independently -L¹-R¹⁹;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R^{1g})O, C(R^{1f})(R^{1g})N(R^{1c}), and C(R^{1f})(R^{1g});
each R^{1e}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or Rif and R¹⁸ are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ.

R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
V is selected from N, N(R¹⁶), C(R¹⁶)(R^{16a}), and C(R¹⁶);
R¹⁶ and R^{16a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR^{12a}, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R^{12a} is independently selected from hydrogen, C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
W is N, N(R¹⁸), C(R¹⁸)(R^{18a}), or C(R¹⁸);
R¹⁸ and R^{18a} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R² is -L³-R^{12c};
L³ is selected from C₁-C₆alkyl, C₂₋₆alkenyl, C₂₋₆alkynyl, —O—, —N(R¹⁴)—, and —S—;
R^{12c} is independently selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R^{20d};
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C_{1- 9}heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

Embodiment C74 A compound of Formula (IV), or a pharmaceutically acceptable salt or solvate thereof:

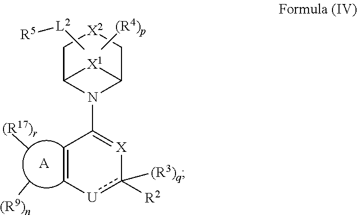

wherein:
ring A is a 7-membered cycloalkyl ring or a 7-membered heterocycloalkyl ring;
X is C(R³) or N;
U is C(R³), C(R³)₂, S(O), S(O)₂, C(O), N(R³), or N;
X¹ is selected from CH₂, C(R⁴)(R⁶), C═N—OR⁴, C═NN(R⁴)(R⁶), C(O)N(R⁴), N(R⁴), N(R⁶), O, S, S(O), S(═O)(═NR⁴), S(O)₂N(R⁴), N(R⁴)S(O)N(R⁴), N(R⁴)S(O)₂N(R⁴), S(O)N(R⁴), OC(O)N(R⁴), N(R⁴)C(O)N(R⁴), S(O)₂, CH₂C(R⁴)(R⁶), CH₂C(R⁴)(R⁶)CH₂, C(R⁴)(R⁶)C(R⁴)(R⁶)C(R⁴)(R⁶), C(R⁴)(R⁶)C═N—OR⁴, CH₂C═NN(R⁴)(R⁶), C(R⁴)(R⁶)C(O)N(R⁴), C(R⁴)(R⁶)N(R⁴), C(R⁴)(R⁶)N(R⁶), C(R⁴)(R⁶)O, C(R⁴)(R⁶)OC(R⁴)(R⁶), C(R⁴)(R⁶)S, C(R⁴)(R⁶)SC(R⁴)(R⁶), C(R⁴)(R⁶)S(O), C(R⁴)(R⁴)S(O)C(R⁴)(R⁶), C(R⁴)(R⁶)S(O)₂C(R⁴)(R⁶), C(R⁴)(R⁶)S(═O)(═NR⁴), C(R⁴)(R⁶)S(O)₂N(R⁴), C(R⁴)(R⁶)N(R⁴)S(O)N(R⁴), C(R⁴)(R⁶)N(R⁴)S(O)₂N(R⁴), C(R⁴)(R⁶)S(O)N(R⁴), C(R⁴)(R⁶)OC(O)N(R⁴), C(R⁴)(R⁶)N(R⁴)C(O)N(R⁴), C(R⁴)(R⁶)S(O)₂, C═NN(R⁴)(R⁶)C(R⁴)(R⁶), C(O)N(R⁴)C(R⁴)(R⁶), S(O)₂N(R⁴)C(R⁴)(R⁶), S(O)N(R⁴)C(R⁴)(R⁶), OC(O)N(R⁴)C(R⁴)(R⁶), C(R⁴)(R⁴), C═N—OR⁴, C═NN(R⁴)(R⁴), CH₂C(R⁴)(R⁴), CH₂C(R⁴)(R⁴)CH₂, C(R⁴)(R⁴)C(R⁴)(R⁶)C(R⁴)(R⁴), C(R⁴)(R⁴)C(R⁴)(R⁴)C(R⁴)(R⁴), C(R⁴)(R⁴)C═N—OR⁴, CH₂C═NN(R⁴)(R⁴), C(R⁴)(R⁴)C(O)N(R⁴), C(R⁴)(R⁴)N(R⁴), C(R⁴)(R⁴)N(R⁶), C(R⁴)(R⁴)O, C(R⁴)(R⁴)OC(R⁴)(R⁴), C(R⁴)(R⁴)S, C(R⁴)(R⁴)SC(R⁴)(R⁴), C(R⁴)(R⁴)S(O), C(R⁴)(R⁴)S(O)C(R⁴)(R⁴), C(R⁴)(R⁴)S(O)₂C(R⁴)(R⁴), C(R⁴)(R⁴)S(═O)(═NR⁴), C(R⁴)(R⁴)S(O)₂N(R⁴), C(R⁴)(R⁴)N(R⁴)S(O)N(R⁴), C(R⁴)(R⁴)N(R⁴)S(O)₂N(R⁴), C(R⁴)(R⁴)S(O)N(R⁴), C(R⁴)(R⁴)OC(O)N(R⁴), C(R⁴)(R⁴)N(R⁴)C(O)N(R⁴), C(R⁴)(R⁴)S(O)₂, C═NN(R⁴)(R⁴)C(R⁴)(R⁴), C(O)N(R⁴)C(R⁴)(R⁴), S(O)₂N(R⁴)C(R⁴)(R⁴), S(O)N(R⁴)C(R⁴)(R⁴), and OC(O)N(R⁴)C(R⁴)(R⁴);
X² is selected from N(R¹), N(R⁶), O, S, S(O), —C(H)(R⁶)—, and —C(R⁴)₂—;
R¹ is selected from hydrogen, C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₁₋₆haloalkyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20a}; R² is -L³-R^{12b};
L³ is selected from C₁-C₆alkyl, C₂₋₆alkenyl, C₂₋₆alkynyl, —O—, —N(R¹⁴)—, and —S—;
each R³ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
q is 0 or 1;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
p is 0, 1, 2, 3, 4, or 5;
R⁶ is -L²-R⁵;
each L² is independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R⁵ is independently hydrogen, or a group other than an electrophilic moiety capable of forming a covalent bond with the cysteine residue at position 12 of a KRAS protein;
R¹⁷ is independently -L¹-R¹⁹;
r is 0 or 1;
L¹ is selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g});
each R^{1c}, R^{1f}, and R^{1g} is independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ; or Rif and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a monocyclic C₃₋₁₀cycloalkyl, monocyclic C₂₋₉heterocycloalkyl, phenyl, and monocyclic C₁₋₅heteroaryl, wherein the monocyclic C₃₋₁₀cycloalkyl, monocyclic C₂₋₉heterocycloalkyl, phenyl, and monocyclic C₁₋₅heteroaryl are optionally substituted with one, two, three, four, five, six, or seven R¹ⁱ;

R¹ⁱ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ, each R⁹ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(H)(R¹²), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
n is 0, 1, 2, 3, 4, 5, 6, 7, 8, 9, or 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
R^{12b} is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
each R^{20a}, R^{20b}, R^{20c}, R^{20d}, R^{20e}, R^{20f}, and R²⁰ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁-haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
[symbol] indicates a single or double bond such that all valences are satisfied.

Embodiment C75 The compound of embodiment C73 or embodiment C74, or a pharmaceutically acceptable salt or solvate thereof, wherein R¹⁹ is selected from

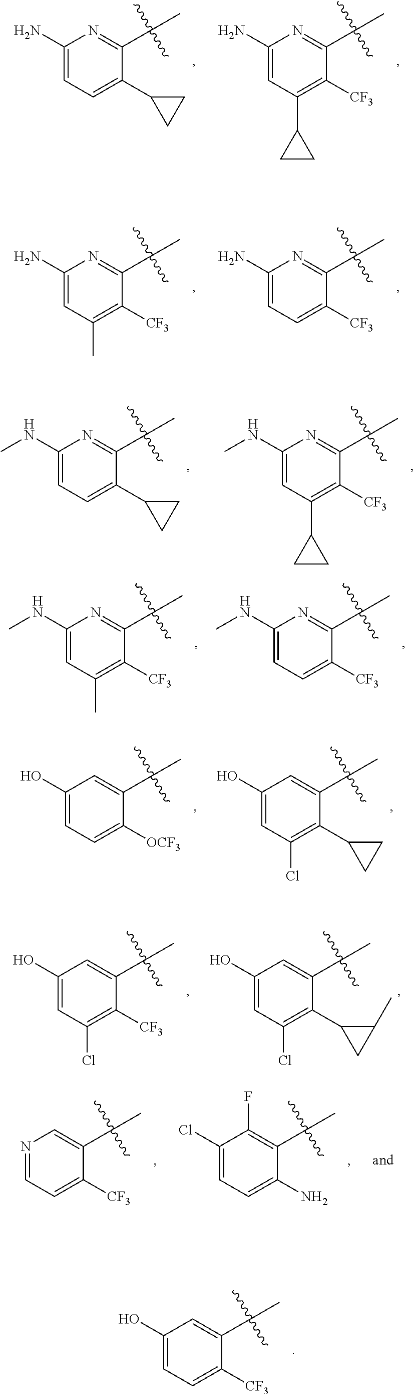

Embodiment D1 A compound of Formula (I), or a pharmaceutically acceptable salt or solvate thereof:

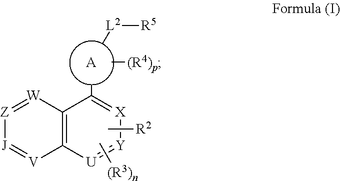

wherein:

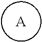

is a 7- or 8-membered monocyclic heterocycloalkyl ring;
X is C or N;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};

R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment D2 The compound of Embodiment D1, or a pharmaceutically acceptable salt or solvate thereof, having the structure of Formula (I′):

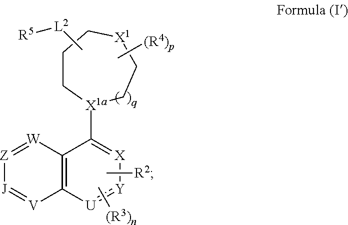

wherein X¹ is selected from C, N, O, S, S(O), and S(O)₂; X^{1a} is selected from N and C(H); and q is 1 or 2.

Embodiment D3 The compound of Embodiment D1 or Embodiment D2, or a pharmaceutically acceptable salt or solvate thereof, having the structure of Formula (Ia):

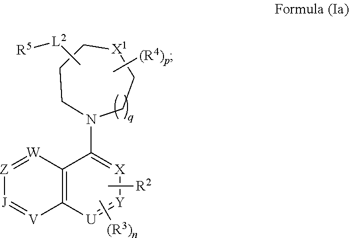

wherein X¹ is selected from C, N, O, S, S(O), and S(O)₂; and q is 1 or 2.

Embodiment D4 The compound of Embodiment D3, or a pharmaceutically acceptable salt or solvate thereof, having the structure of Formula (Ib):

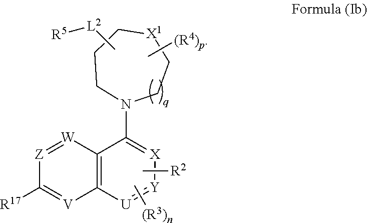

Embodiment D5 The compound of Embodiment D4, or a pharmaceutically acceptable salt or solvate thereof, having the structure of Formula (Ic):

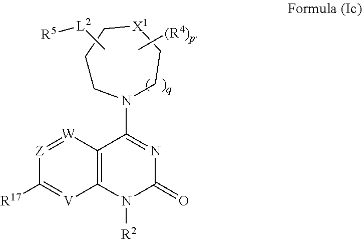

Embodiment D6 The compound of Embodiment D4, or a pharmaceutically acceptable salt or solvate thereof, having the structure of Formula (Id):

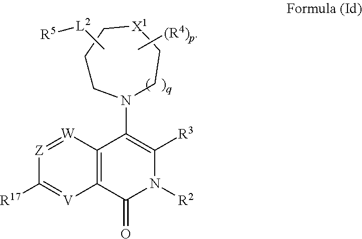

Embodiment D7 The compound of Embodiment D4, or a pharmaceutically acceptable salt or solvate thereof, having the structure of Formula (Ie):

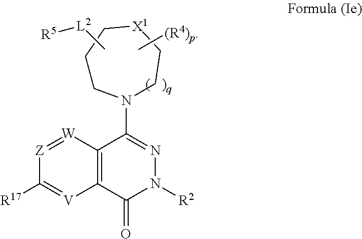

Embodiment D8 The compound of Embodiment D4, or a pharmaceutically acceptable salt or solvate thereof, having the structure of Formula (If):

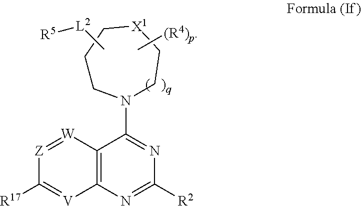

Embodiment D9 The compound of Embodiment D4, or a pharmaceutically acceptable salt or solvate thereof, having the structure of Formula (Ig):

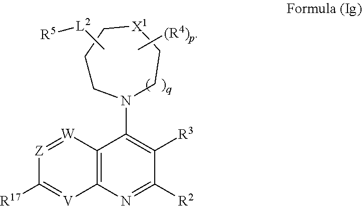

Embodiment D10 A compound of Formula (I″), or a pharmaceutically acceptable salt or solvate thereof:

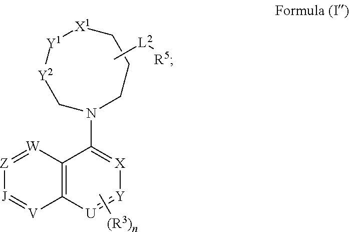

wherein:
X is C or N;
X¹ is selected from C(R⁴)(R⁶), N(R¹²), N(R⁶), O, S, S(O), and S(O)₂;
Y is C(R⁷), S(O), S(O)₂, C(O), or N;
Y¹ is selected from CH₂, N(H), O, S, S(O), and S(O)₂;
Y² is selected from a bond, CH₂, N(H), O, S, S(O), and S(O)₂;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};

R⁵ is hydrogen, or a group other than an electrophilic moiety capable of forming a covalent bond with the cysteine residue at position 12 of a KRAS protein;
R⁶ is -L²-R⁵;
R⁷ is selected from halogen, —CN, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹⁵), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b}; or R⁷ is C₁₋₆alkyl substituted with one, two, or three R^{20b};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, Co-10aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2; and

[symbol] indicates a single or double bond such that all valences are satisfied.

Embodiment D11 The compound of Embodiment D10, or a pharmaceutically acceptable salt or solvate thereof, wherein Y² is a bond.

Embodiment D21 The compound of Embodiment D10, or a pharmaceutically acceptable salt or solvate thereof, wherein Y² is CH₂.

Embodiment D13 The compound of any one of Embodiments D10-D12, or a pharmaceutically acceptable salt or solvate thereof, wherein Y¹ is CH₂.

Embodiment D14 The compound of any one of Embodiments D2-D13, or a pharmaceutically acceptable salt or solvate thereof, wherein X¹ is N(H).

Embodiment D15 The compound of any one of Embodiments D2-D13, or a pharmaceutically acceptable salt or solvate thereof, wherein X¹ is N(R⁴).

Embodiment D16 The compound of any one of Embodiments D2-D13, or a pharmaceutically acceptable salt or solvate thereof, wherein X¹ is N(-L²-R⁵).

Embodiment D17 The compound of any one of Embodiments D2-D13, or a pharmaceutically acceptable salt or solvate thereof, wherein X¹ is O.

Embodiment D18 The compound of any one of Embodiments D2-D13, or a pharmaceutically acceptable salt or solvate thereof, wherein X¹ is S.

Embodiment D19 The compound of any one of Embodiments D2-D13, or a pharmaceutically acceptable salt or solvate thereof, wherein X¹ is S(O)₂.

Embodiment D20 The compound of any one of Embodiments D2-D13, or a pharmaceutically acceptable salt or solvate thereof, wherein X¹ is CH₂.

Embodiment D21 The compound of any one of Embodiments D2-D13, or a pharmaceutically acceptable salt or solvate thereof, wherein X¹ is C(H)(R⁴).

Embodiment D22 The compound of any one of Embodiments D2-D13, or a pharmaceutically acceptable salt or solvate thereof, wherein X¹ is C(R⁴)₂.

Embodiment D23 The compound of any one of Embodiments D2-D13, or a pharmaceutically acceptable salt or solvate thereof, wherein X¹ is C(H)(-L²-R⁵).

Embodiment D24 The compound of any one of Embodiments D2-D23, or a pharmaceutically acceptable salt or solvate thereof, wherein q is 1.

Embodiment D25 The compound of any one of Embodiments D2-D23, or a pharmaceutically acceptable salt or solvate thereof, wherein q is 2.

Embodiment D26 The compound of any one of Embodiments D1-D25, or a pharmaceutically acceptable salt or solvate thereof, wherein V is C(R¹⁶).

Embodiment D27 The compound of any one of Embodiments D1-D26, or a pharmaceutically acceptable salt or solvate thereof, wherein V is C(H).

Embodiment D28 The compound of any one of Embodiments D1-D25, or a pharmaceutically acceptable salt or solvate thereof, wherein V is N.

Embodiment D29 The compound of any one of Embodiments D1-D28, or a pharmaceutically acceptable salt or solvate thereof, wherein W is C(R¹⁸).

Embodiment D30 The compound of Embodiment D29, or a pharmaceutically acceptable salt or solvate thereof, wherein W is C(H).

Embodiment D31 The compound of any one of Embodiments D1-D28, or a pharmaceutically acceptable salt or solvate thereof, wherein W is N.

Embodiment D32 The compound of any one of Embodiments D1-D31, or a pharmaceutically acceptable salt or solvate thereof, wherein Z is C(R⁸).

Embodiment D33 The compound of Embodiment D32, or a pharmaceutically acceptable salt or solvate thereof, wherein Z is C(H).

Embodiment D34 The compound of any one of Embodiments D1-D31, or a pharmaceutically acceptable salt or solvate thereof, wherein Z is N.

Embodiment D35 A compound of Formula (II), or a pharmaceutically acceptable salt or solvate thereof:

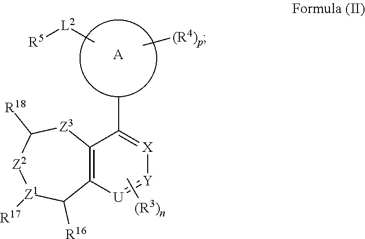

wherein:

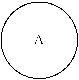

is a 7- or 8-membered monocyclic heterocycloalkyl ring;
X is C or N;
Y is C, S(O), S(O)₂, C(O), or N;
U is C, S(O), S(O)₂, C(O), or N;
W is N or C(R¹⁸);
Z¹ is N or C(R⁶);
Z² is N(R⁷) or C(R⁸)(R⁹);
Z³ is absent, N(R²⁶), or C(R²⁷)(R²⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a}; or two R⁴ on the same carbon atom are combined to form a C₃₋₆cycloalkyl optionally substituted with one, two, or three R^{20a}; or two R⁴ on adjacent carbon atoms are combined to form a C₃₋₆cycloalkyl, C₂₋₉heterocycloalkyl, C₆₋₁₀aryl, or C₁₋₉heteroaryl, wherein the C₃₋₆cycloalkyl, C₂₋₉heterocycloalkyl, C₆₋₁₀aryl, or C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is selected from hydrogen and C₁₋₆alkyl;
R⁷ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, Co-10aryl, and C₁₋₉heteroaryl are optionally substituted with one, two, or three R^{20c};
R⁹ is selected from hydrogen and C₁₋₆alkyl;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, R²⁰ⁱ, and R^{20j} are each independently selected from halogen, —CN, C₁₋₆alkyl, C₂₋₆ alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹², —C(O)R¹⁵, —S(O)R¹⁵, —C(O)N(R¹²)(R¹³), —C(O)C(O)N(R¹²)(R¹³), —S(O)₂R¹⁵, and —S(O)₂N(R¹²)(R¹³)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
R²⁷ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═OX(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃-cycloalkyl, C₂₋₉heterocycloalkyl, C₆₋₁₀aryl, and C₁₋₉heteroaryl are optionally substituted with one, two, or three R^{20j};
R²⁸ is selected from hydrogen and C₁₋₆alkyl;
n is 0, 1, or 2;
p is 1, 2, 3, 4, or 5; and
[symbol] indicates a single or double bond such that all valences are satisfied.

Embodiment D36 The compound of Embodiment D35, or a pharmaceutically acceptable salt or solvate thereof, having the structure of Formula (IIa):

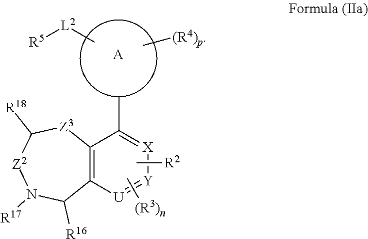

Embodiment D37 The compound of Embodiment D35, or a pharmaceutically acceptable salt or solvate thereof, having the structure of Formula (IIb):

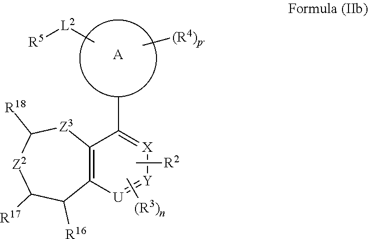

Embodiment D38 The compound of Embodiment D35 or Embodiment D36, or a pharmaceutically acceptable salt or solvate thereof, having the structure of Formula (IIc′):

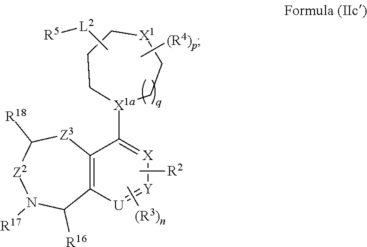

wherein X¹ is selected from C, N, O, S, S(O), and S(O)₂; X^{1a} is selected from N and C(H); and q is 1 or 2.

Embodiment D39 The compound of Embodiment D38, or a pharmaceutically acceptable salt or solvate thereof, having the structure of Formula (IIc):

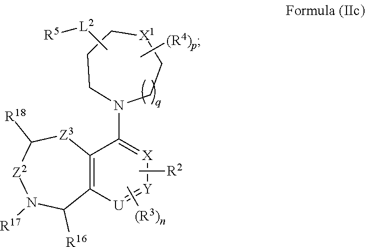

wherein X¹ is selected from C, N, O, S, S(O), and S(O)₂; and q is 1 or 2.

Embodiment D40 The compound of Embodiment D35 or Embodiment D37, or a pharmaceutically acceptable salt or solvate thereof, having the structure of Formula (IId′):

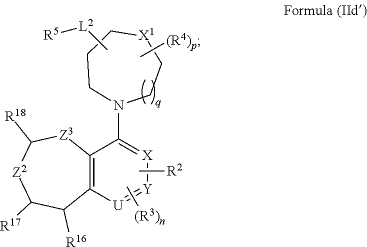

wherein X¹ is selected from C, N, O, S, S(O), and S(O)₂; X^{1a} is selected from N and C(H); and q is 1 or 2.

Embodiment D41 The compound of Embodiment D40, or a pharmaceutically acceptable salt or solvate thereof, having the structure of Formula (IId):

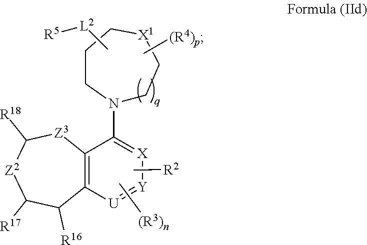

wherein X¹ is selected from C, N, O, S, S(O), and S(O)₂; and q is 1 or 2.

Embodiment D42 The compound of any one of Embodiments D38-D41, or a pharmaceutically acceptable salt or solvate thereof, wherein X¹ is N(H).

Embodiment D43 The compound of any one of Embodiments D38-D41, or a pharmaceutically acceptable salt or solvate thereof, wherein X¹ is N(R⁴).

Embodiment D44 The compound of any one of Embodiments D38-D41, or a pharmaceutically acceptable salt or solvate thereof, wherein X¹ is N(-L²-R⁵).

Embodiment D45 The compound of any one of Embodiments D38-D41, or a pharmaceutically acceptable salt or solvate thereof, wherein X¹ is O.

Embodiment D46 The compound of any one of Embodiments D38-D41, or a pharmaceutically acceptable salt or solvate thereof, wherein X¹ is S.

Embodiment D47 The compound of any one of Embodiments D38-D41, or a pharmaceutically acceptable salt or solvate thereof, wherein X¹ is S(O)₂.

Embodiment D48 The compound of any one of Embodiments D38-D41, or a pharmaceutically acceptable salt or solvate thereof, wherein X¹ is CH₂.

Embodiment D49 The compound of any one of Embodiments D38-D41, or a pharmaceutically acceptable salt or solvate thereof, wherein X¹ is C(H)(R⁴).

Embodiment D50 The compound of any one of Embodiments D38-D41, or a pharmaceutically acceptable salt or solvate thereof, wherein X¹ is C(R⁴)₂.

Embodiment D51 The compound of any one of Embodiments D38-D41, or a pharmaceutically acceptable salt or solvate thereof, wherein X¹ is C(H)(-L²-R⁵).

Embodiment D52 The compound of any one of Embodiments D38-D51, or a pharmaceutically acceptable salt or solvate thereof, wherein q is 1.

Embodiment D53 The compound of any one of Embodiments D38-D51, or a pharmaceutically acceptable salt or solvate thereof, wherein q is 2.

Embodiment D54 The compound of any one of Embodiments D35-D53, or a pharmaceutically acceptable salt or solvate thereof, wherein Z³ is absent.

Embodiment D55 The compound of any one of Embodiments D35-D53, or a pharmaceutically acceptable salt or solvate thereof, wherein Z² is C(R⁸)(R⁹).

Embodiment D56 The compound of any one of Embodiments D35-D55, or a pharmaceutically acceptable salt or solvate thereof, wherein R⁹ is hydrogen.

Embodiment D57 The compound of any one of Embodiments D35-D55, or a pharmaceutically acceptable salt or solvate thereof, wherein X is C.

Embodiment D58 The compound of any one of Embodiments D35-D55, or a pharmaceutically acceptable salt or solvate thereof, wherein X is N.

Embodiment D59 The compound of any one of Embodiments D35-D58, or a pharmaceutically acceptable salt or solvate thereof, wherein Y is C.

Embodiment D60 The compound of any one of Embodiments D35-D58, or a pharmaceutically acceptable salt or solvate thereof, wherein Y is N.

Embodiment D61 The compound of any one of Embodiments D35-D58, or a pharmaceutically acceptable salt or solvate thereof, wherein Y is C(O).

Embodiment D62 The compound of any one of Embodiments D35-D61, or a pharmaceutically acceptable salt or solvate thereof, wherein U is C.

Embodiment D63 The compound of any one of Embodiments D35-D61, or a pharmaceutically acceptable salt or solvate thereof, wherein U is N.

Embodiment D64 The compound of any one of Embodiments D35-D60, or a pharmaceutically acceptable salt or solvate thereof, wherein U is C(O).

Embodiment D65 The compound of any one of Embodiments D42-D53, or a pharmaceutically acceptable salt or solvate thereof, having the structure of Formula (IIe):

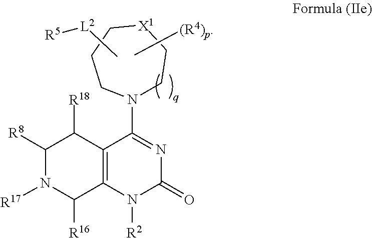

Embodiment D66 The compound of any one of Embodiments D42-D53, or a pharmaceutically acceptable salt or solvate thereof, having the structure of Formula (IIf):

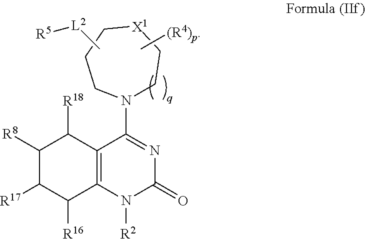

Embodiment D67 The compound of any one of Embodiments D42-D53, or a pharmaceutically acceptable salt or solvate thereof, having the structure of Formula (IIg):

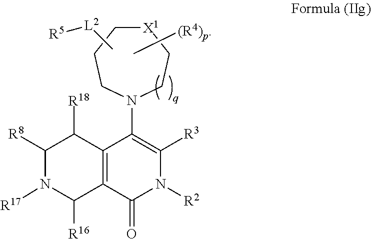

Embodiment D68 The compound of any one of Embodiments D42-D53, or a pharmaceutically acceptable salt or solvate thereof, having the structure of Formula (IIh):

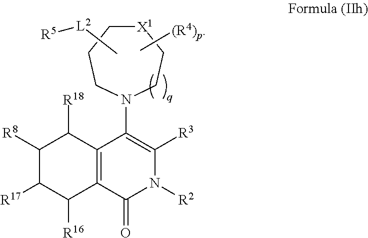

Embodiment D69 The compound of any one of Embodiments D42-D53, or a pharmaceutically acceptable salt or solvate thereof, having the structure of Formula (Ii):

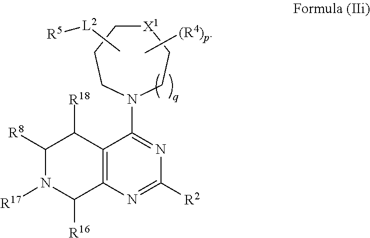

Embodiment D70 The compound of any one of Embodiments D42-D53, or a pharmaceutically acceptable salt or solvate thereof, having the structure of Formula (IIj):

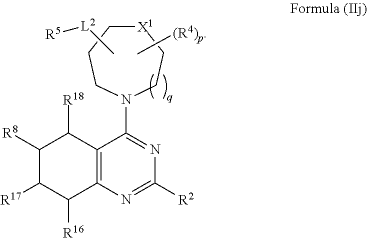

Embodiment D71 The compound of any one of Embodiments D42-D53, or a pharmaceutically acceptable salt or solvate thereof, having the structure of Formula (IIk):

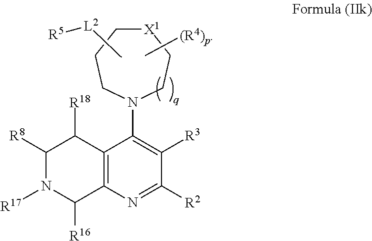

Embodiment D72 The compound of any one of Embodiments D42-D53, or a pharmaceutically acceptable salt or solvate thereof, having the structure of Formula (IIm):

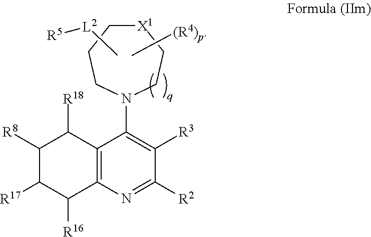

Embodiment D73 A compound of Formula (IIIa)-(IIIi), or a pharmaceutically acceptable salt or solvate thereof:

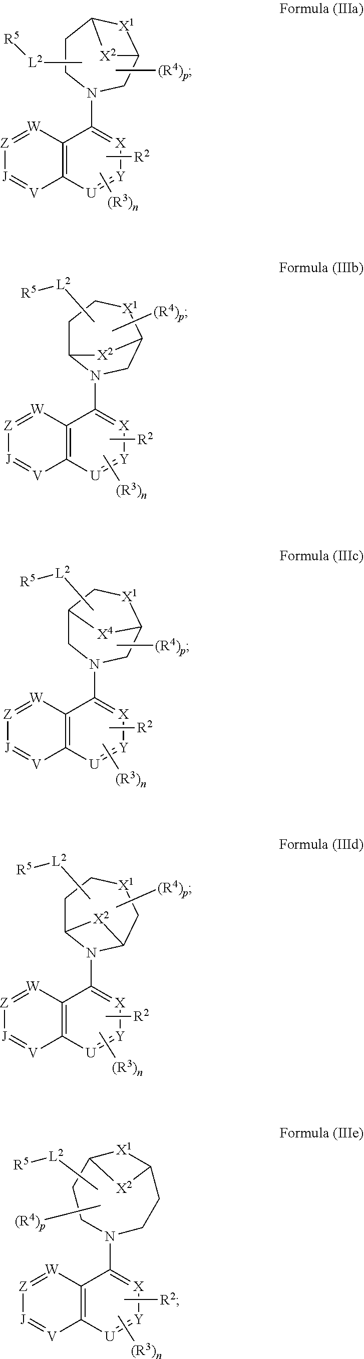

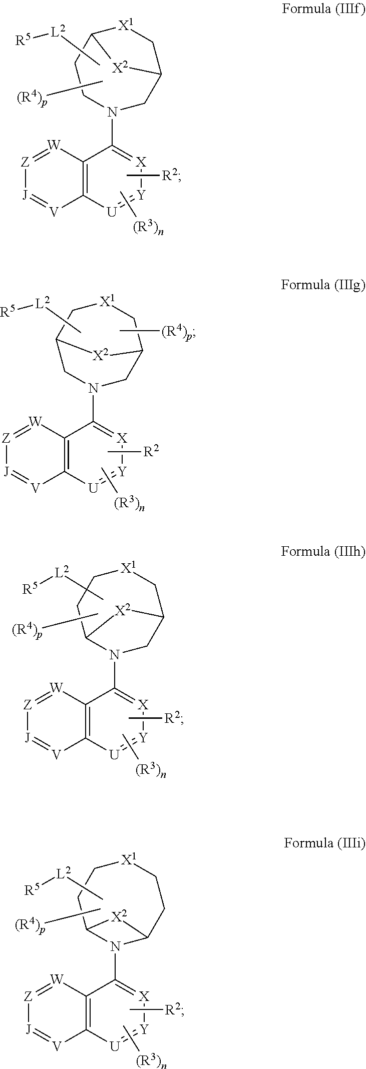

X¹ is selected from C(R⁴)(R⁶), N(R⁴), N(R⁶), O, S, S(O), and S(O)₂;
X² is selected from X³, —CH₂—, —X³CH₂—, —CH₂X³—, —CH₂CH₂—, —C(H)(R⁴)—, —C(H)(R⁴)—, —X³C(H)(R⁴)—, —C(H)(R⁴)X³—, —C(H)(R⁴)CH₂—, —C(H)(R⁴)C(H)(R⁴)—, —C(R⁴)₂—, —X³C(R⁴)₂—, —C(R⁴)₂X³—, —C(H)C(R⁴)₂—, —C(R⁴)₂C(H)—, and —C(R⁴)₂C(R⁴)₂—;
X³ is selected from N(R¹), O, S, S(O), and S(O)₂;
X⁴ is selected from X³, —CH₂—, —X³CH₂—, —CH₂X³—, —CH₂CH₂—, —C(H)(R⁴)—, —C(H)(R⁴)—, —X⁵—, —C(H)(R⁴)—, —C(H)(R⁴)X³—, —C(H)(R⁴)CH₂—, —C(H)(R⁴)C(H)(R⁴)—, —C(R⁴)₂—, —XC(R⁴)₂—, —C(R⁴)₂X³—, —C(H)C(R⁴)₂—, —C(R⁴)₂C(H)—, and —C(R⁴)₂C(R⁴)₂—;
X⁵ is selected from N(R¹), S, S(O), and S(O)₂;
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁶ is -L²-R⁵;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20c}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁-haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5; and
[symbol] indicates a single or double bond such that all valences are satisfied.

Embodiment D74 The compound of Embodiment D73, or a pharmaceutically acceptable salt or solvate thereof, having the structure of Formula (IIIa):

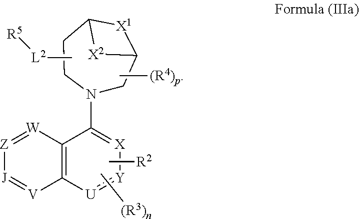

Embodiment D75 The compound of Embodiment D73, or a pharmaceutically acceptable salt or solvate thereof, having the structure of Formula (IIIb):

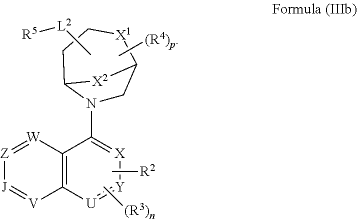

Embodiment D76 The compound of Embodiment D73, or a pharmaceutically acceptable salt or solvate thereof, having the structure of Formula (IIId):

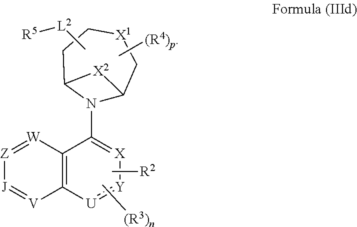

Embodiment D77 The compound of Embodiment D73, or a pharmaceutically acceptable salt or solvate thereof, having the structure of Formula (IIIe):

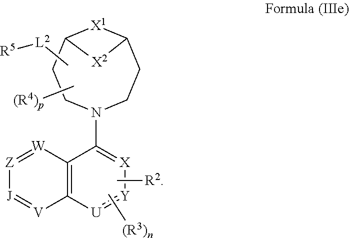

Embodiment D78 The compound of Embodiment D73, or a pharmaceutically acceptable salt or solvate thereof, having the structure of Formula (IIIf):

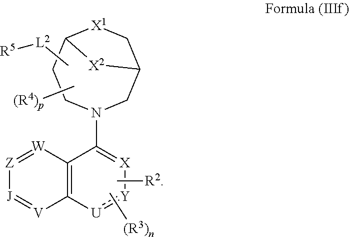

Embodiment D79 The compound of Embodiment D73, or a pharmaceutically acceptable salt or solvate thereof, having the structure of Formula (IIIg):

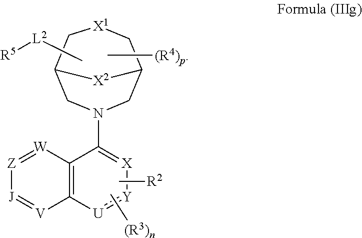

Embodiment D80 The compound of Embodiment D73, or a pharmaceutically acceptable salt or solvate thereof, having the structure of Formula (IIIh):

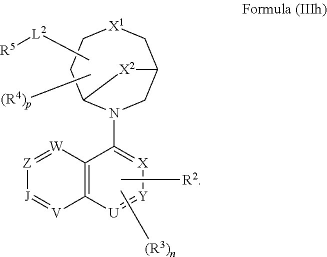

Embodiment D81 The compound of Embodiment D73, or a pharmaceutically acceptable salt or solvate thereof, having the structure of Formula (IIIi):

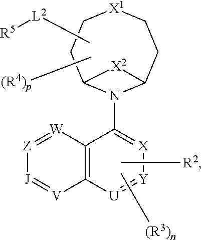

### Formula (IIIi).

Embodiment D82 The compound of Embodiment D73, or a pharmaceutically acceptable salt or solvate thereof, having the structure of Formula (IIIc):

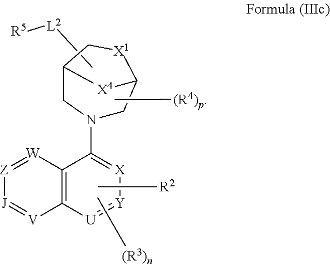

Embodiment D83 The compound of Embodiment D82, or a pharmaceutically acceptable salt or solvate thereof, wherein X⁴ is —NH—.

Embodiment D84 The compound of any one of Embodiments D73-D81, or a pharmaceutically acceptable salt or solvate thereof, wherein X² is selected from —CH₂— and —CH₂CH₂—.

Embodiment D85 A compound of Formula (IVa), (IVb), or (IVc), or a pharmaceutically acceptable salt or solvate thereof:

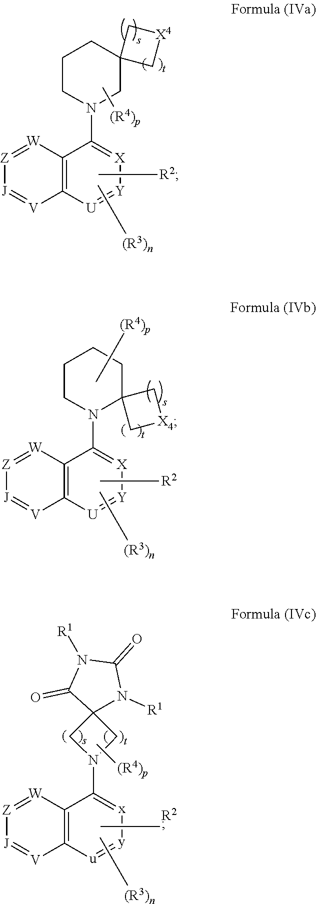

wherein:
X is C or N;
X⁴ is selected from N(R¹), O, S, S(O), S(O)₂, —CH₂—, —C(H)(R⁴)—, —C(R⁴)₂—, and C(H)(-L²-R⁵);
Y is C, S(O), S(O)₂, C(O), or N;
U is C, S(O), S(O)₂, C(O), or N;
Z is N or C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—;
each R¹ is independently from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a};
R² is selected from hydrogen, halogen, —CN, C₁₋₆alkyl, C₂₋₆alkenyl, C₂₋₆alkynyl, C₃₋₆cycloalkyl, C₂₋₉heterocycloalkyl, Co-10aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
s is 0, 1, 2, 3, or 4;
t is 1, 2, 3, 4, or 5; wherein s+t≥2; and
[symbol] indicates a single or double bond such that all valences are satisfied.

Embodiment D86 The compound of Embodiment D85, or a pharmaceutically acceptable salt or solvate thereof, having the structure of Formula (IVa):

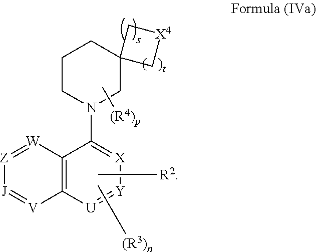

Embodiment D87 The compound of Embodiment D85, or a pharmaceutically acceptable salt or solvate thereof, having the structure of Formula (IVb):

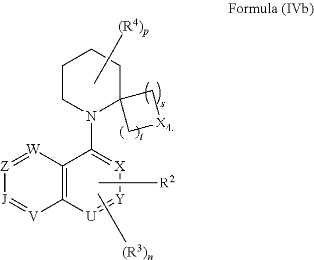

Embodiment D88 The compound of any one of Embodiments D85-D87, or a pharmaceutically acceptable salt or solvate thereof, wherein s is 1 or 2.

Embodiment D89 The compound of any one of Embodiments D85-D88, or a pharmaceutically acceptable salt or solvate thereof, wherein t is 1 or 2.

Embodiment D90 The compound of any one of Embodiments D85-D89, or a pharmaceutically acceptable salt or solvate thereof, wherein X⁴ is N(R¹).

Embodiment D91 The compound of Embodiment D85, or a pharmaceutically acceptable salt or solvate thereof, having the structure of Formula (IVc):

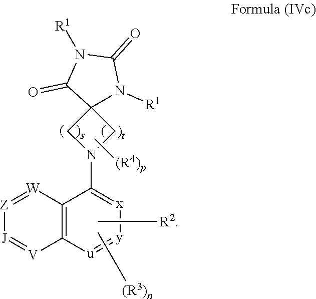

Embodiment D92 A compound of Formula (Va), (Vb), or (Vc), or a pharmaceutically acceptable salt or solvate thereof:

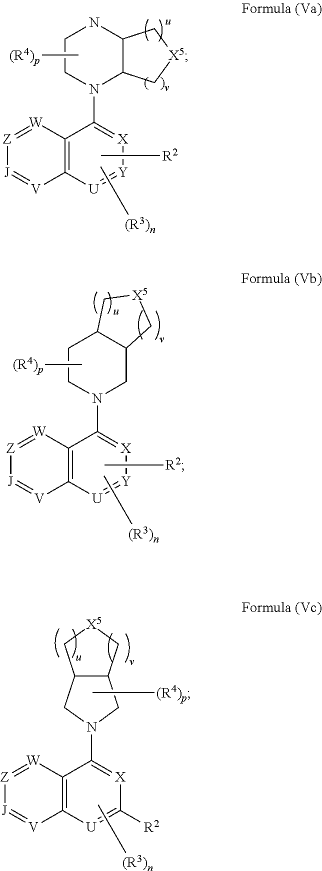

X is C or N;
X⁵ is selected from N(R¹), O, S, S(O), and S(O)₂;
Y is C, S(O), S(O)₂, C(O), or N;
U is C, S(O), S(O)₂, C(O), or N;
Z is N or C(R⁸);
Z¹ is C(R⁸);
V and J are independently selected from N, C(R¹⁶), and C(R¹⁷), wherein one of V and J is C(R¹⁷);
W is N or C(R¹⁸);
L¹ and L² are independently selected from a bond, C₁-C₆alkyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, and —N(R¹⁴)C(O)N(R¹⁴)—; R¹ is selected from hydrogen, -L²-R⁵, —C(O)OR¹², —C(O)R¹⁵, —C(O)N(R¹²)(R¹³), —S(O)₂R¹⁵, —S(O)₂N(R¹²)(R¹³)—, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20a}; R² is selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
each R³ is independently selected from hydrogen, halogen, oxo, C₁-C₆alkyl, C₁-C₆haloalkyl, —OR¹², —N(R¹²)(R¹³), —CN, —C(O)OR¹², —OC(O)N(R¹²)(R¹³), —C(O)R¹⁵, —S(O)₂R¹⁵, and —S(O)₂N(R¹²)(R¹³);
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is hydrogen, or a group other than an electrophilic moiety capable of forming a covalent bond with the cysteine residue at position 12 of a KRAS protein;
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20c};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, Co-10aryl, and C₁₋₉heteroaryl are optionally substituted with one, two, or three R^{20g};
R¹⁷ is -L¹-R¹⁹;
R¹⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R¹⁹ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, C₆₋₁₀aryl, and C₁₋₉heteroaryl are optionally substituted with one, two, or three R²⁰ⁱ;
each R^{20a}, R^{20b}, R^{20c}, R^{20d}, R^{20e}, R^{20f}, R^{20g}, R^{20h}, and R²⁰ⁱ are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
n is 0, 1, or 2;
p is 1, 2, 3, 4, or 5;
u is 0, 1, 2, 3, or 4;
v is 0, 1, 2, 3, or 4; and
[symbol] indicates a single or double bond such that all valences are satisfied.

Embodiment D93 The compound of Embodiment D92, or a pharmaceutically acceptable salt or solvate thereof, having the structure of Formula (Va):

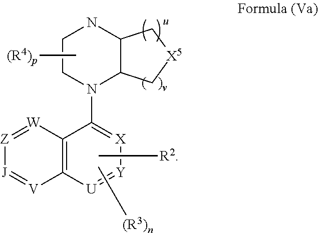

Embodiment D94 The compound of Embodiment D92, or a pharmaceutically acceptable salt or solvate thereof, having the structure of Formula (Vb):

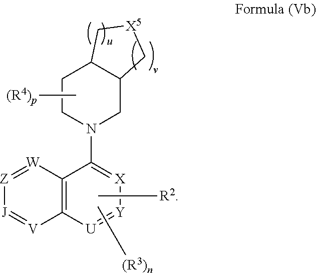

Embodiment D95 The compound of Embodiment D92, or a pharmaceutically acceptable salt or solvate thereof, having the structure of Formula (Vc):

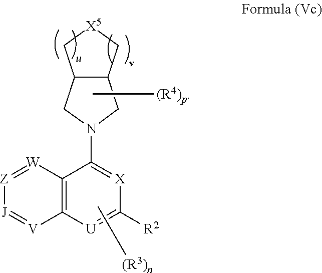

Embodiment D96 The compound of any one of Embodiments D92-D95, or a pharmaceutically acceptable salt or solvate thereof, wherein u is 0 or 1.

Embodiment D97 The compound of any one of Embodiments D92-D95, or a pharmaceutically acceptable salt or solvate thereof, wherein v is 0 or 1.

Embodiment D98 The compound of any one of Embodiments D92-D97, or a pharmaceutically acceptable salt or solvate thereof, wherein X⁵ is N(R¹).

Embodiment D99 The compound of any one of Embodiments D85-D98, or a pharmaceutically acceptable salt or solvate thereof, wherein R¹ is hydrogen.

Embodiment D100 The compound of any one of Embodiments D84-D98, or a pharmaceutically acceptable salt or solvate thereof, wherein R¹ is -L²-R⁵.

Embodiment D101 The compound of any one of Embodiments D73-D100, or a pharmaceutically acceptable salt or solvate thereof, wherein Y is C.

Embodiment D102 The compound of any one of Embodiments D73-D100, or a pharmaceutically acceptable salt or solvate thereof, wherein Y is N.

Embodiment D103 The compound of any one of Embodiments D73-D100, or a pharmaceutically acceptable salt or solvate thereof, wherein Y is C(O).

Embodiment D104 The compound of any one of Embodiments D73-D103, or a pharmaceutically acceptable salt or solvate thereof, wherein X is C.

Embodiment D105 The compound of any one of Embodiments D73-D103, or a pharmaceutically acceptable salt or solvate thereof, wherein X is N.

Embodiment D106 The compound of any one of Embodiments D73-D105, or a pharmaceutically acceptable salt or solvate thereof, wherein U is C.

Embodiment D107 The compound of any one of Embodiments D73-D105, or a pharmaceutically acceptable salt or solvate thereof, wherein U is N.

Embodiment D108 The compound of any one of Embodiments D73-D105, or a pharmaceutically acceptable salt or solvate thereof, wherein U is C(O).

Embodiment D109 The compound of any one of Embodiments D73-D108, or a pharmaceutically acceptable salt or solvate thereof, wherein Z is C(R⁸).

Embodiment D110 The compound of any one of Embodiments D73-D109, or a pharmaceutically acceptable salt or solvate thereof, wherein R⁸ is hydrogen.

Embodiment D111 The compound of any one of Embodiments D73-D108, or a pharmaceutically acceptable salt or solvate thereof, wherein Z is N.

Embodiment D112 The compound of any one of Embodiments D73-D111, or a pharmaceutically acceptable salt or solvate thereof, wherein V is C(R¹⁶).

Embodiment D113 The compound of any one of Embodiments D73-D112, or a pharmaceutically acceptable salt or solvate thereof, wherein V is C(H).

Embodiment D114 The compound of any one of Embodiments D73-D111, or a pharmaceutically acceptable salt or solvate thereof, wherein V is N.

Embodiment D115 The compound of any one of Embodiments D73-D114, or a pharmaceutically acceptable salt or solvate thereof, wherein J is C(R¹⁷).

Embodiment D116 The compound of any one of Embodiments D73-D115, or a pharmaceutically acceptable salt or solvate thereof, wherein W is C(R¹⁸).

Embodiment D117 The compound of Embodiment D116, or a pharmaceutically acceptable salt or solvate thereof, wherein W is C(H).

Embodiment D118 The compound of any one of Embodiments D73-D117, or a pharmaceutically acceptable salt or solvate thereof, wherein W is N.

Embodiment D119 The compound of any one of Embodiments D1-D118, or a pharmaceutically acceptable salt or solvate thereof, wherein R² is selected from hydrogen, C₁₋₆alkyl, C₃₋₆cycloalkyl, C₂₋₉heterocycloalkyl, C₆₋₁₀aryl, C₁₋₉heteroaryl, —OR¹², —SR¹², and —N(R¹²)(R¹³), wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{20b}.

Embodiment D120 The compound of any one of Embodiments D1-D119, or a pharmaceutically acceptable salt or solvate thereof, wherein R² is selected from —OR¹², —SR¹², and C₁₋₆alkyl, wherein C₁₋₆alkyl is optionally substituted with one, two, or three R^{20b}.

Embodiment D121 The compound of any one of Embodiments D1-D120, or a pharmaceutically acceptable salt or solvate thereof, wherein R² is —OR¹².

Embodiment D122 The compound of any one of Embodiments D1-D121, or a pharmaceutically acceptable salt or solvate thereof, wherein R¹² is selected from C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d}.

Embodiment D123 The compound of any one of Embodiments D1-D121, or a pharmaceutically acceptable salt or solvate thereof, wherein R¹² is C₁₋₆alkyl optionally substituted with one, two, or three R^{20d}.

Embodiment D124 The compound of any one of Embodiments D1-D121, or a pharmaceutically acceptable salt or solvate thereof, wherein R¹² is C₂₋₉heterocycloalkyl optionally substituted with one, two, or three R^{20d}.

Embodiment D125 The compound of any one of Embodiments D1-D121, or a pharmaceutically acceptable salt or solvate thereof, wherein R¹² is —CH₂—C₂₋₉heterocycloalkyl optionally substituted with one, two, or three R^{20d}.

Embodiment D126 The compound of any one of Embodiments D1-D125, or a pharmaceutically acceptable salt or solvate thereof, wherein each R^{20d} is independently selected from halogen, C₁₋₆alkyl, C₃₋₆cycloalkyl, C₂₋₉heterocycloalkyl, C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₃₋₆cycloalkyl, C₂₋₉heterocycloalkyl, C₆₋₁₀aryl, C₁₋₉heteroaryl are optionally substituted with one, two, or three groups independently selected from halogen, oxo, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —S(O)₂R²⁵, and —S(O)₂N(R²²)(R²³).

Embodiment D127 The compound of any one of Embodiments D1-D126, or a pharmaceutically acceptable salt or solvate thereof, wherein each R^{20d} is independently selected from halogen, C₁₋₆alkyl, and —OR²¹, wherein C₁₋₆alkyl is optionally substituted with one, two, or three groups independently selected from halogen, oxo, C₁₋₆alkyl, —OR²¹, and —N(R²²)(R²³).

Embodiment D128 The compound of any one of Embodiments D1-D119, or a pharmaceutically acceptable salt or solvate thereof, wherein R² is selected from

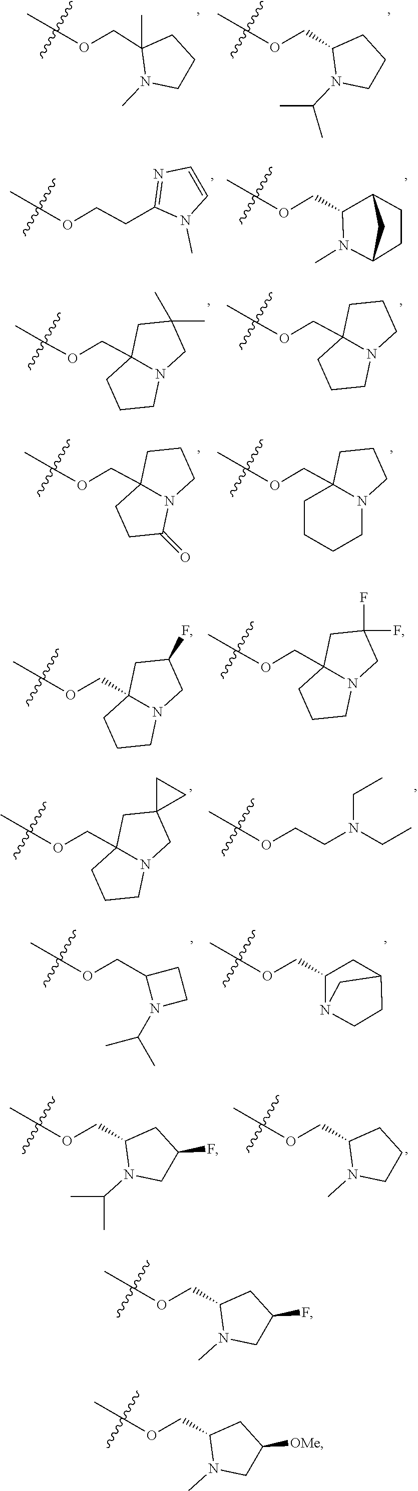

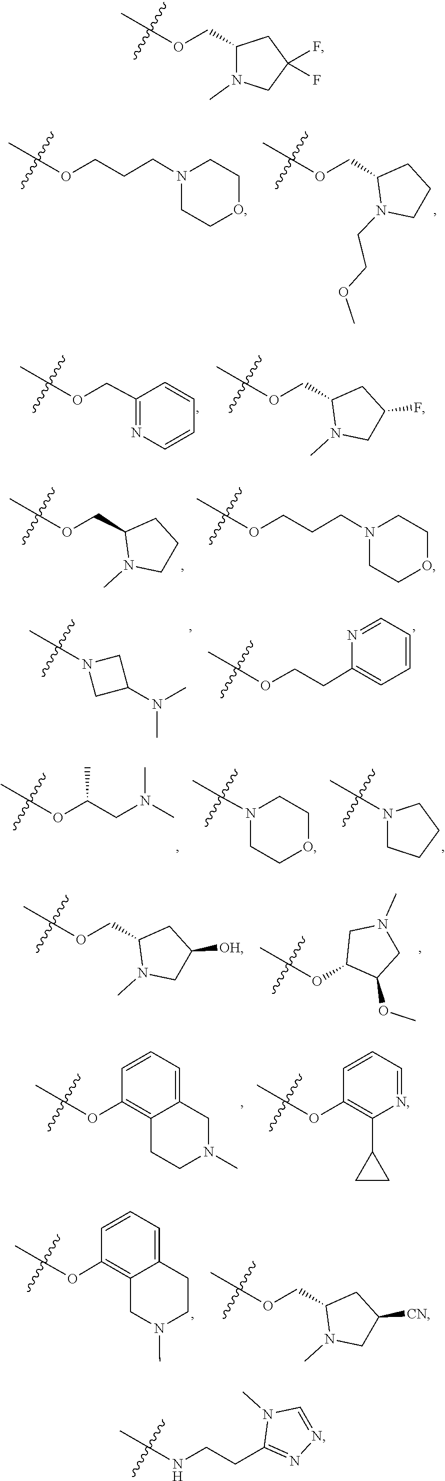

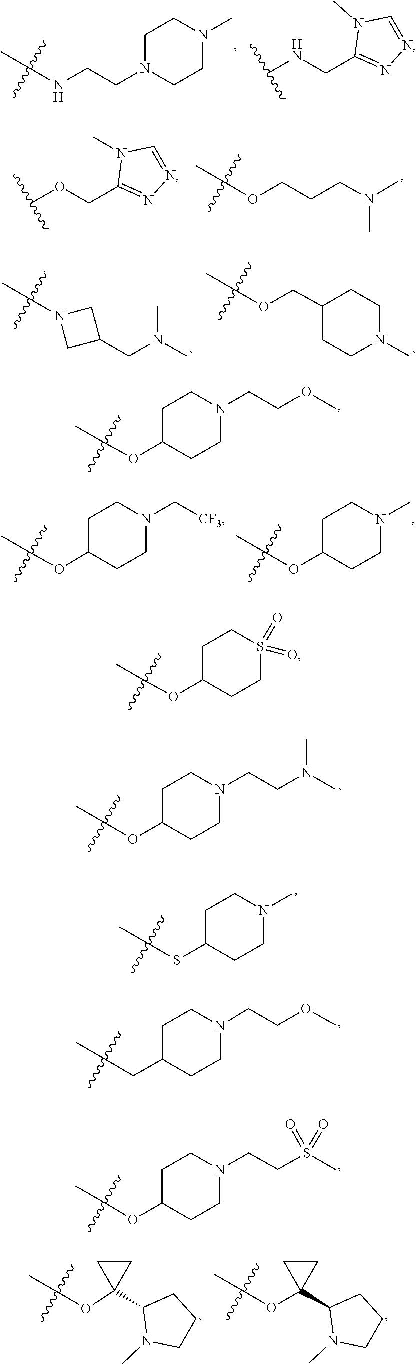

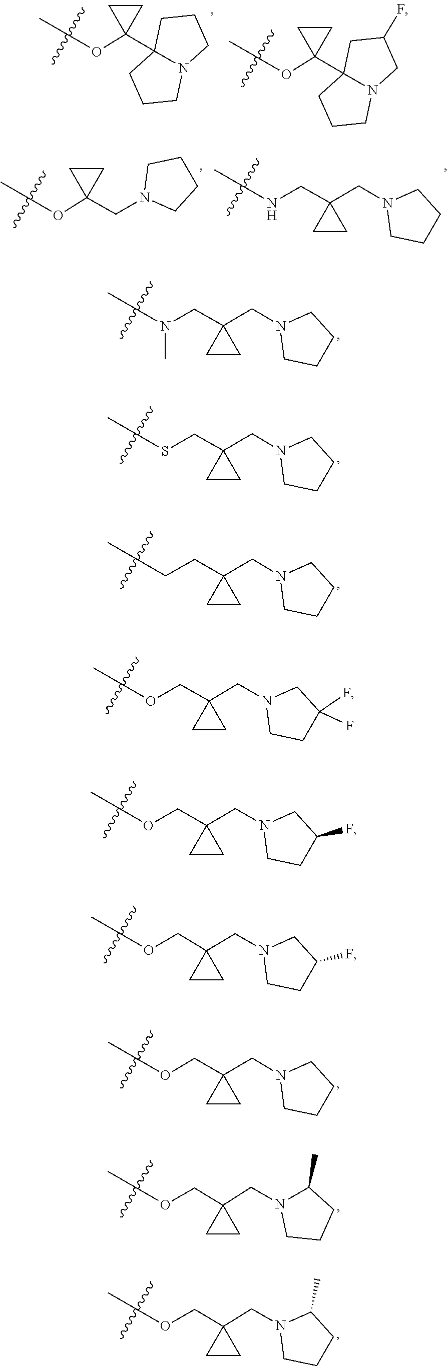

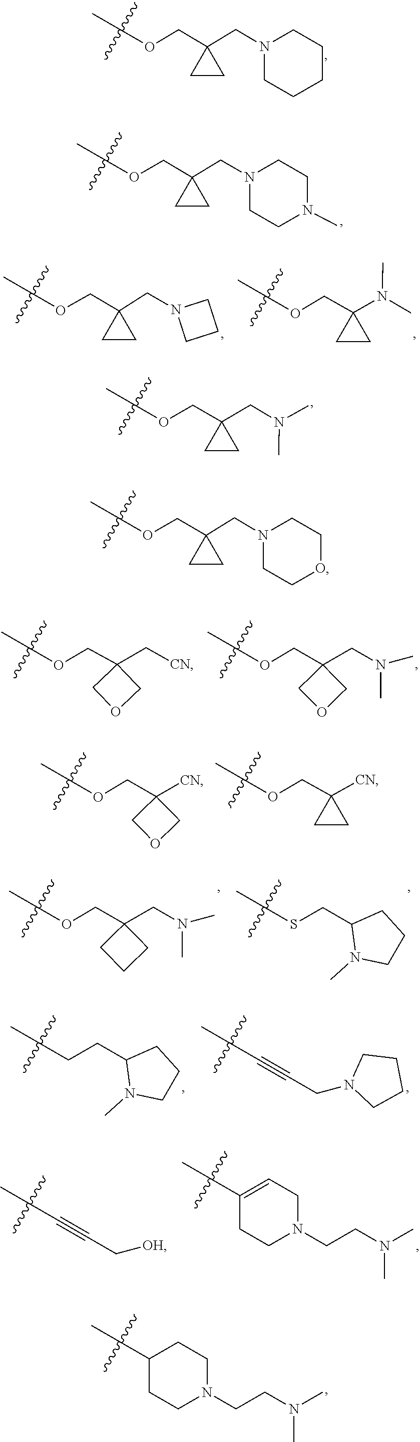

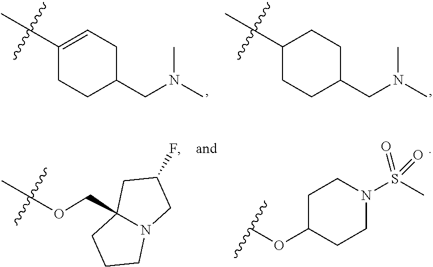 [SMILES: *C1=CCC(CN(C)C)CC1 ; *C1CCC(CN(C)C)CC1 ; *OC[C@]12CCCN1C[C@@H](F)C2 ; *OC1CCN(S(C)(=O)=O)CC1]

Embodiment D129 The compound of any one of Embodiments D1-D128, or a pharmaceutically acceptable salt or solvate thereof, wherein L¹ is a bond.

Embodiment D130 The compound of any one of Embodiments D1-D128, or a pharmaceutically acceptable salt or solvate thereof, wherein L¹ is O.

Embodiment D131 The compound of any one of Embodiments D1-D130, or a pharmaceutically acceptable salt or solvate thereof, wherein R¹⁹ is C₁₋₉heteroaryl optionally substituted with one, two, or three R²⁰ⁱ.

Embodiment D132 The compound of any one of Embodiments D1-D130, or a pharmaceutically acceptable salt or solvate thereof, wherein R¹⁹ is C₆₋₁₀aryl optionally substituted with one, two, or three R²⁰ⁱ.

Embodiment D133 The compound of any one of Embodiments D1-D132, or a pharmaceutically acceptable salt or solvate thereof, wherein L² is selected from a bond, C₁-C₆alkyl, and —C(O)—.

Embodiment D134 The compound of any one of Embodiments D1-D133, or a pharmaceutically acceptable salt or solvate thereof, wherein L² is a bond.

Embodiment D135 The compound of any one of Embodiments D1-D133, or a pharmaceutically acceptable salt or solvate thereof, wherein L² is a C₁-C₆alkyl.

Embodiment D136 The compound of any one of Embodiments D1-D135, or a pharmaceutically acceptable salt or solvate thereof, wherein R⁵ is hydrogen.

Embodiment D137 The compound of any one of Embodiments D1-D135, or a pharmaceutically acceptable salt or solvate thereof, wherein R⁵ is a group other than an electrophilic moiety capable of forming a covalent bond with the cysteine residue at position 12 of a KRAS protein.

Embodiment D138 The compound of Embodiment D138, or a pharmaceutically acceptable salt or solvate thereof, wherein R⁵ is —H, —NH₂, —OH, —NH(C₁₋₆ alkyl),

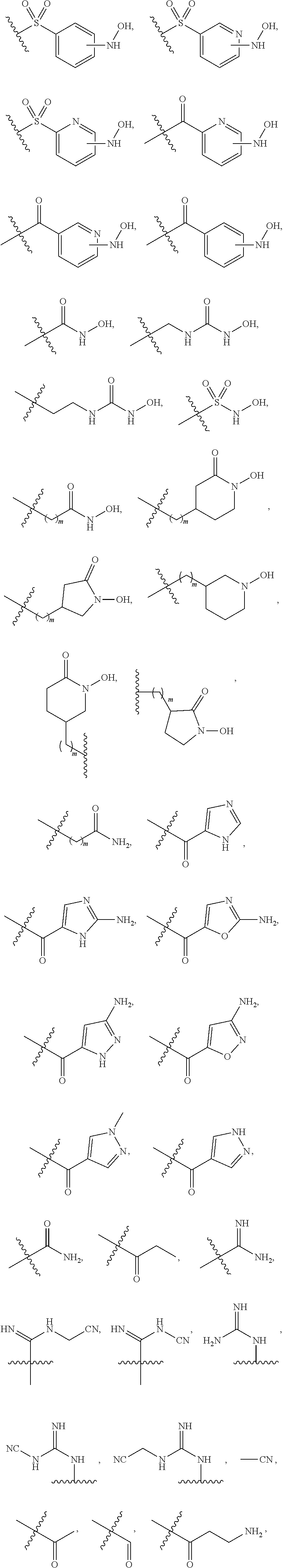

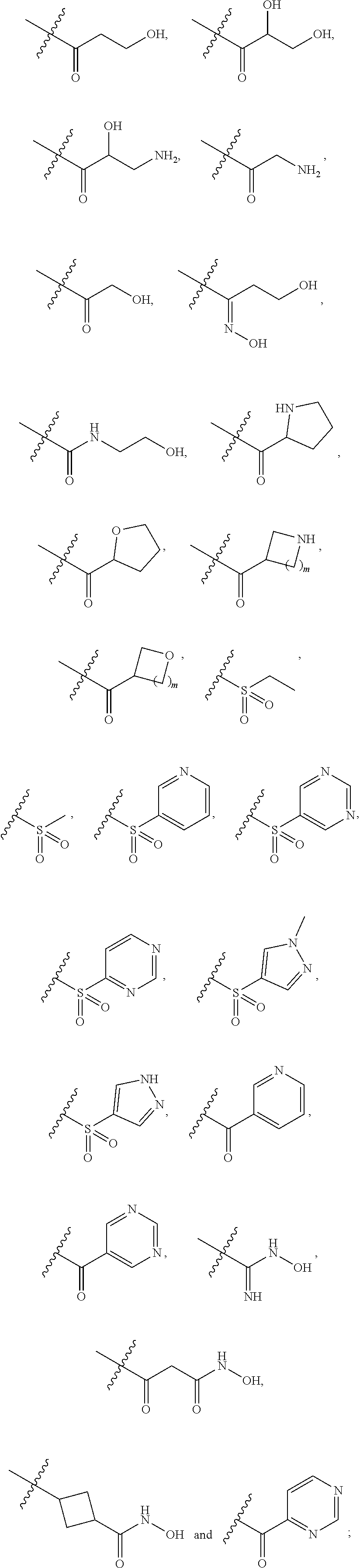

and m, when present, is 0, 1, 2, or 3.

Embodiment D139 A compound selected from:

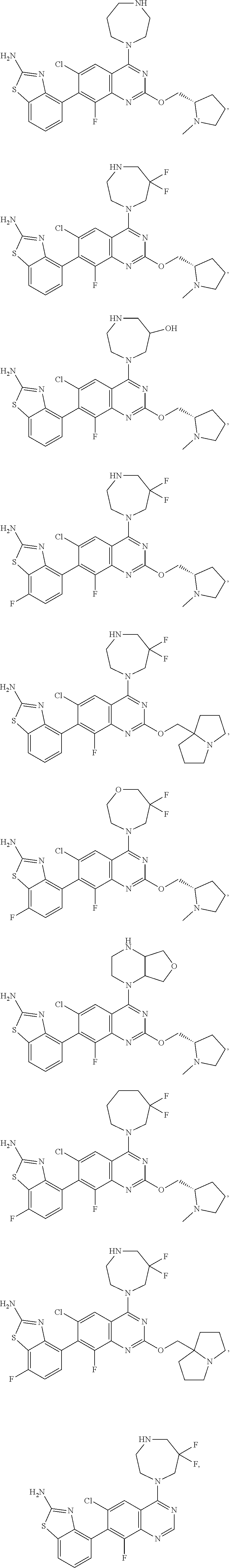

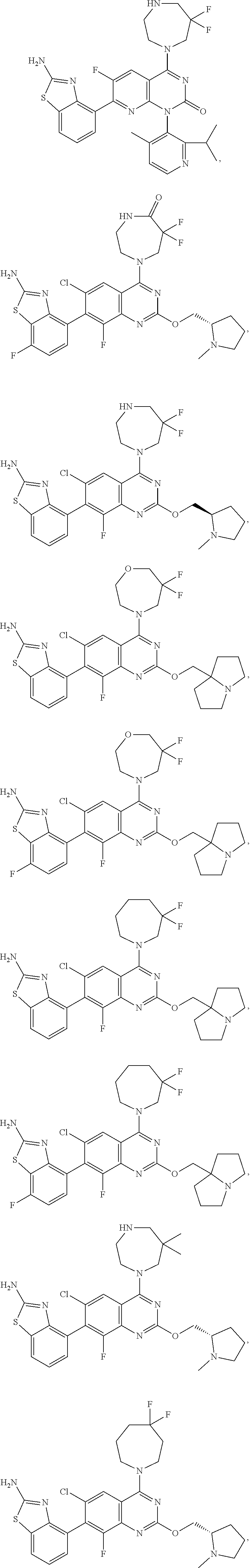

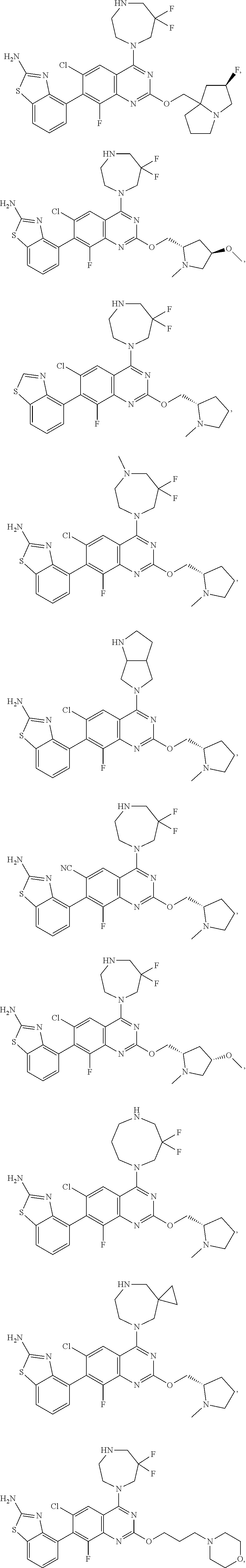

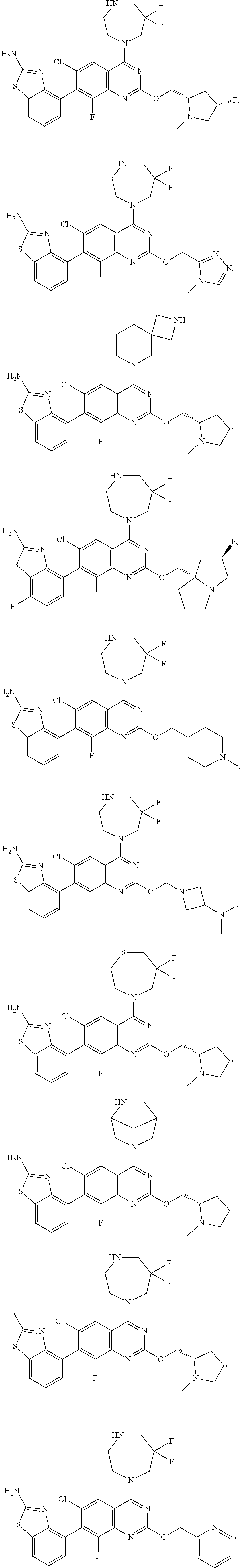

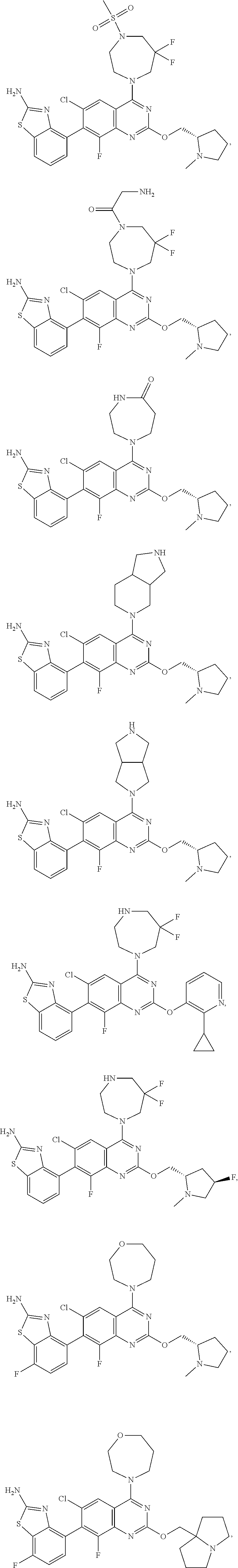

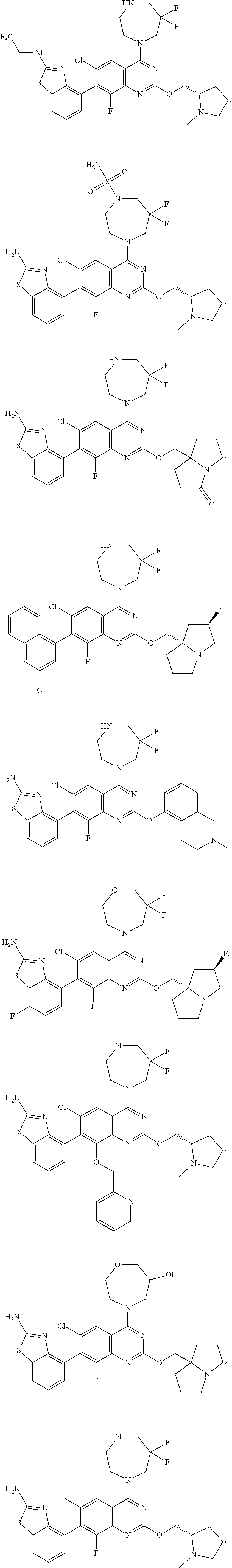

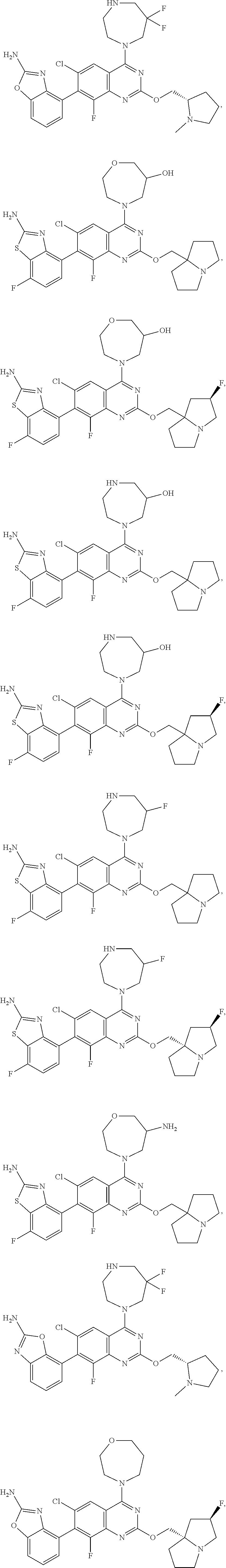

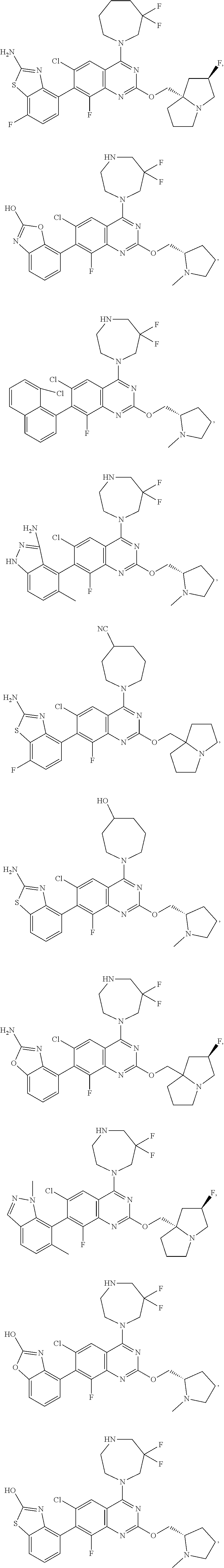

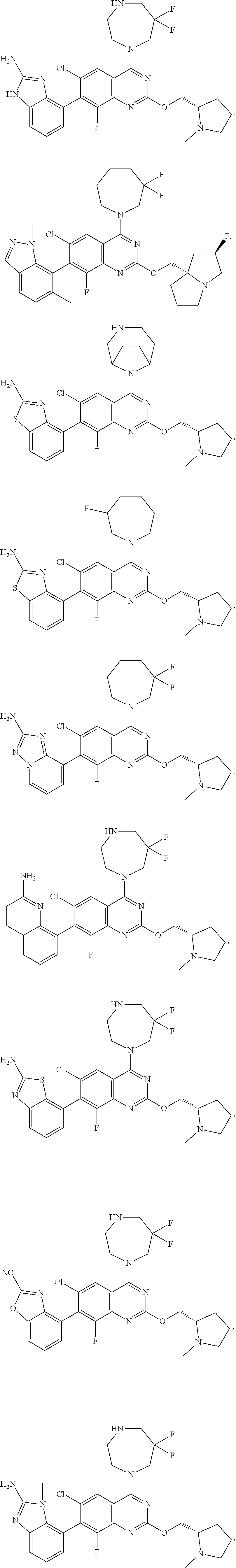

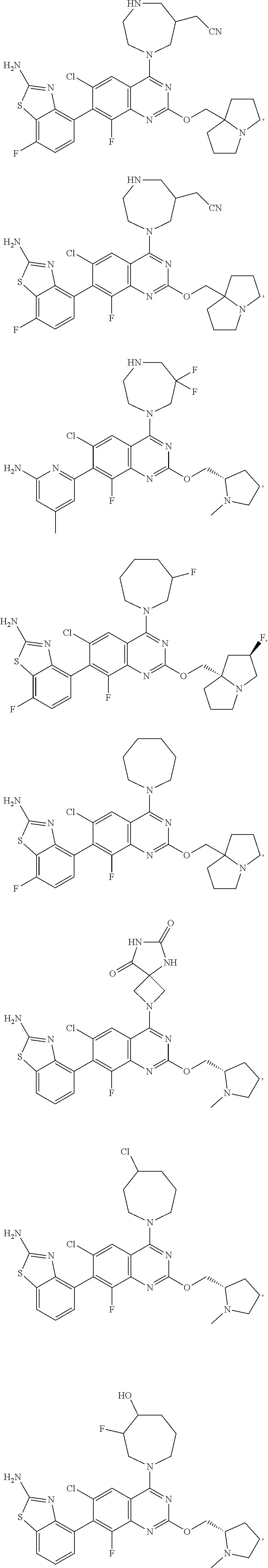

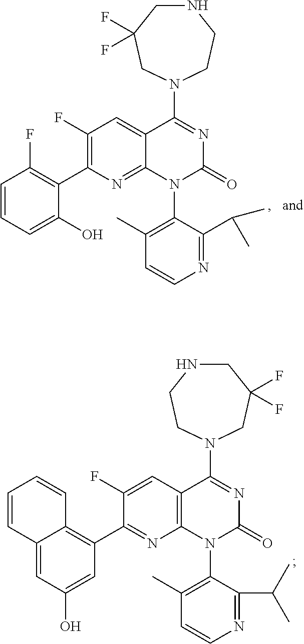 [SMILES: Cc1ccnc(C(C)C)c1-n1c(=O)nc(N2CCNCC(F)(F)C2)c2cc(F)c(-c3c(O)cccc3F)nc21 ; Cc1ccnc(C(C)C)c1-n1c(=O)nc(N2CCNCC(F)(F)C2)c2cc(F)c(-c3cc(O)cc4ccccc34)nc21]

or a pharmaceutically acceptable salt or solvate thereof.

Embodiment D140 A pharmaceutical composition comprising a compound of any one of Embodiments D1-D139, or a pharmaceutically acceptable salt or solvate thereof, and a pharmaceutically acceptable excipient.

Embodiment D141 A method of treating cancer in a subject in need thereof, comprising administering to the subject a therapeutically effective amount of a compound of any one of Embodiments D1-D139, or a pharmaceutically acceptable salt or solvate thereof.

Embodiment D142 The method of Embodiment D141, wherein the cancer is a solid tumor.

Embodiment D143 The method of Embodiment D141, wherein the cancer is a hematological cancer.

Embodiment D144 A method of modulating activity of a Ras protein, comprising contacting a Ras protein with an effective amount of a compound of any one of Embodiments D1-D139, or a pharmaceutically acceptable salt or solvate thereof, thereby modulating the activity of the Ras protein.

Embodiment D145 The method of Embodiment D144, wherein said modulating comprises inhibiting the Ras protein activity.

Embodiment D146 The method of Embodiment D144, wherein the Ras protein is a K-Ras protein.

Embodiment D147 The method of Embodiment D144, wherein the Ras protein is a G12D or G12V mutant K-Ras.

Embodiment D148 The method of any one of Embodiments D141-D147 comprising administering an additional agent or therapy.

Embodiment D149 The method of Embodiment D148, wherein the additional agent or therapy is selected from the group consisting of a chemotherapeutic agent, a radioactive agent, and an immune modulator.

Embodiment D150 The method of Embodiment D144, wherein said modulating takes place in vitro or in vivo.

Embodiment D151 A method of inhibiting cell growth, comprising administering a cell expressing a Ras protein with an effective amount of a compound of any one of Embodiments D1-D139, or a pharmaceutically acceptable salt or solvate thereof, thereby inhibiting growth of said cells.

Embodiment D152 The method of Embodiment D151 comprising administering an additional agent to said cell.

Embodiment D153 The method of Embodiment D152, wherein the additional agent is a chemotherapeutic agent, a radioactive agent, or an immune modulator.

Embodiment D154 A Ras protein bound by a compound of any one of Embodiments D1-D139, or a pharmaceutically acceptable salt or solvate thereof, wherein activity of said Ras protein is reduced as compared to a Ras protein unbound to said compound.

### EXAMPLES

The following examples are provided for illustrative purposes only and not to limit the scope of the claims provided herein.

As used herein, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
i-Bu iso-butyl
t-Bu tert-butyl
DCM dichloromethane (CH₂Cl₂)
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1,1′-bis(diphenylphosphino)ferrocene
EDC or EDCI N-(3-dimethylaminopropyl)-N′-ethylcarbodiimide hydrochloride
eq equivalent(s)
Et ethyl
Et₂O diethyl ether
EtOH ethanol
EtOAc ethyl acetate
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
NMR nuclear magnetic resonance
Ph phenyl
iPr/i-Pr iso-propyl
RP-HPLC reverse-phase high-pressure liquid chromatography
RT room temperature
TBS tert-butyldimethylsilyl
TEA triethylamine
TFA trifluoroacetic acid
THF tetrahydrofuran
TLC thin layer chromatography
TMS trimethylsilyl
TsOH/p-TsOH p-toluenesulfonic acid.

### Example 1A: Synthesis of 4-(6-chloro-4-(1,4-diazepan-1-yl)-8-fluoro-2-(((S)-1-methylpyrrolidin-2-yl)methoxy)quinazolin-7-yl)benzo[d]thiazol-2-amine (Compound 101)

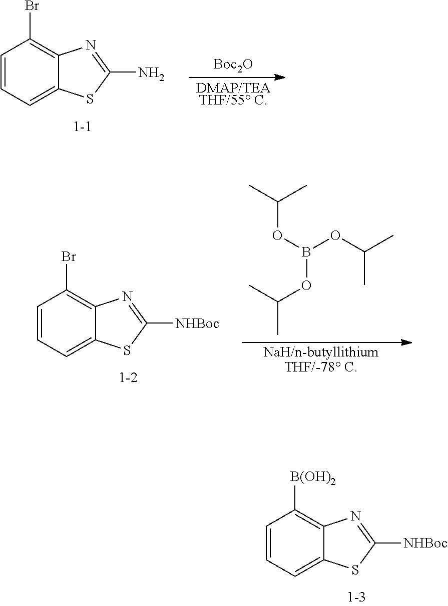

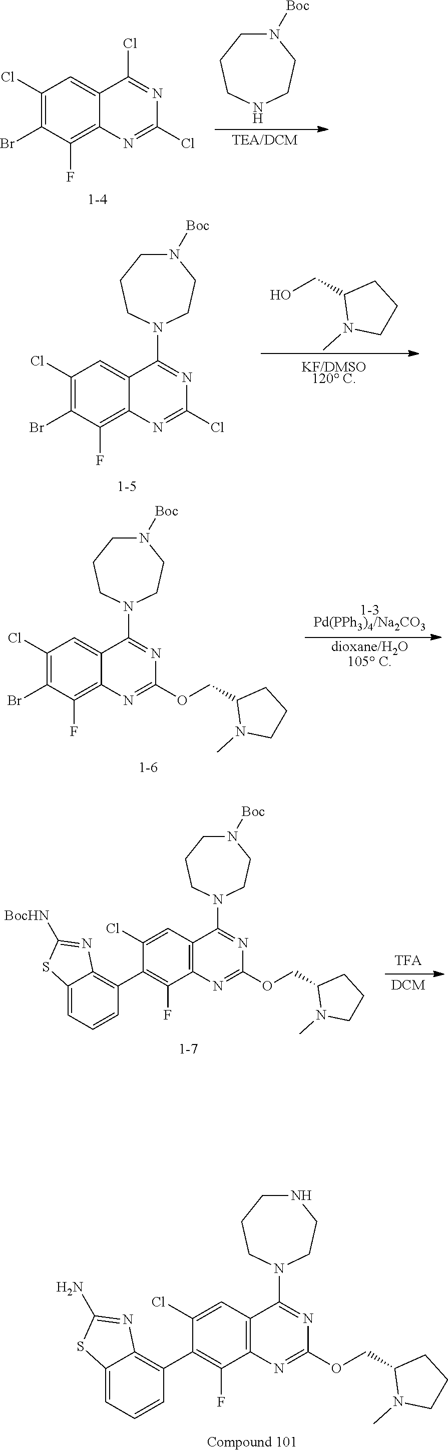

To a solution of 4-bromobenzo[d]thiazol-2-amine (1-1) (2 g, 8.81 mmol) in THF (20 mL), Boc₂O (2.88 g, 13.21 mmol), TEA (2.66 g, 26.43 mmol) and DMAP (107 mg, 0.88 mmol) were added and the resulting mixture was stirred at 55° C. overnight. The mixture was allowed to cool to RT and partitioned between with ethyl acetate and water. The combined organic layer was concentrated in vacuo. The residue was purified on a silica gel column to afford product (1-2) (1.7 g). ESI-MS m/z: 327.99 [M+H]⁺.

To a solution of compound 1-2 (1.7 g, 5.18 mmol) in THF (30 mL) under nitrogen, NaH (60% in paraffin oil, 310 mg) was added in portions and the resulting mixture was stirred for 10 min. The mixture was cooled to −78° C., n-butyllithium (2.5 M in hexanes, 3.1 mL) was added dropwise. The mixture was stirred for 25 min and then triisopropyl borate (4.25 mL) was added dropwise. The mixture was stirred at −78° C. for 25 min and then was allowed to warm to RT. The reaction mixture was quenched with saturated aqueous NH₄Cl, extracted with ethyl acetate, washed with water and brine, dried over Na₂SO₄, filtered and concentrated in vacuo. The residue was purified by flash column chromatography on silica gel to afford product (1-3) (714 mg). ESI-MS m/z: 294.08 [M+H]⁺.

To a stirred solution of compound 1-4 (1.0 g, 3.05 mmol) in DCM (20 mL), TEA (924 mg, 9.15 mmol) and tert-butyl 1,4-diazepane-1-carboxylate (610 mg, 3.05 mmol) were added and the resulting mixture was stirred at RT for 30 min. The mixture was partitioned between water and DCM. The organic layer was washed with brine, dried over Na₂SO₄, filtered and concentrated in vacuo. The residue was purified by flash column chromatography on silica gel to afford product (1-5) (1.35 g). ESI-MS m/z: 492.01 [M+H]⁺.

To a stirred solution of compound 1-5 (1.35 g, 2.74 mmol) in DMSO (20 mL) under nitrogen, KF (1.27 g, 21.92 mmol) and (S)-(1-methylpyrrolidin-2-yl)methanol (630 mg, 5.48 mmol) were added and the resulting mixture was stirred at 120° C. for 3 h. The mixture was partitioned between water and ethyl acetate. The organic layer was washed with brine, dried over Na₂SO₄, filtered and concentrated in vacuo. The residue was purified by flash column chromatography on silica gel to afford product (1-6) (813 mg). ESI-MS m/z: 571.14 [M+H]⁺.

To a stirred solution of compound 1-6 (100 mg, 0.18 mmol) in 1,4-dioxane (8 mL) and H₂O (2 mL), compound 1-3 (102 mg, 0.350 mmol), tetrakis(triphenylphosphine)palladium (20 mg, 0.018 mmol) and Na₂CO₃ (55 mg, 0.53 mmol) were added and the resulting mixture was stirred at 100° C. under nitrogen overnight. The mixture was partitioned between water and ethyl acetate. The organic layer was washed with brine, dried over Na₂SO₄, filtered and concentrated in vacuo. The residue was purified by flash column chromatography on silica gel to afford product (1-7) (85 mg). ESI-MS m/z: 741.29 [M+H]⁺.

To a solution of compound 1-7 (85 mg) in DCM (6 mL) at RT, TFA (2 mL) was added and the resulting mixture was stirred at RT for 30 min. The mixture was concentrated in vacuo to remove the solvent. The residue was purified by flash column chromatography on silica gel to afford product (Compound 101) (8 mg). ESI-MS m/z: 541.18 [M+H]⁺; 1HNMR (400 MHz, CD₃OD): δ 7.86 (s, 1H), 7.53-7.51 (m, 1H), 7.00-6.98 (d, 2H), 4.510 (s, 2H), 4.11-4.09 (d, 2H), 4.05-4.03 (m, 2H), 3.48-3.45 (m, 2H), 3.19-3.16 (m, 3H), 3.13 (s, 2H), 2.87 (s, 3H), 2.22-2.18 (m, 3H), 1.99-1.96 (m, 1H), 1.89 (d, 2H).

### Example 1B: Synthesis of 4-(6,6-difluoro-1,4-diazepan-1-yl)-6-fluoro-7-(2-fluoro-6-hydroxyphenyl)-1-(2-isopropyl-4-methylpyridin-3-yl)pyrido[2,3-d]pyrimidin-2(1H)-one (Compound 195)

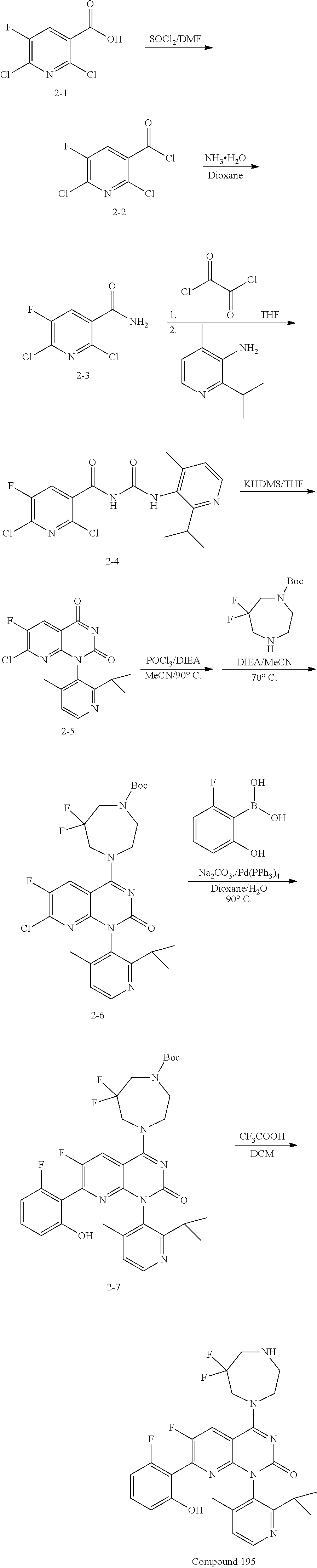

To a solution of 2,6-dichloro-5-fluoronicotinic acid (2-1) (10.0 g, 47.84 mmol) in SOCl₂ (100 mL), DMF (1 drop) was added and the resulting mixture was stirred at 80° ° C. for 16 h. The mixture was allowed to cool to RT and concentrated in vacuo to afford product (2-2) which was used in the next step directly.

To a solution of compound 2-2 in dioxane (100 mL) at 0° ° C., NH₃·H₂O (20 mL) was slowly added (dropwise) and the resulting mixture was stirred at RT for 1 h. The mixture was partitioned between ethyl acetate and water. The organic layer was washed with brine, dried over Na₂SO₄, filtered and concentrated in vacuo. The residue was purified by flash column chromatography on silica gel to afford product (2-3) (8.7 g).

To a stirred solution of compound 2-3 (8.7 g, 41.6 mmol) in THF (100 mL) at 0° C., oxalyl chloride (7.9 g, 62.4 mmol) was added and the resulting mixture was stirred at 75° C. for 1 h. The mixture was allowed to cool to RT and concentrated in vacuo. The residue was dissolved in THF (100 mL), cooled to −78° C., and 2-isopropyl-4-methylpyridin-3-amine (7.5 g, 49.92 mmol) was added. The mixture was stirred at RT for 16 h, poured into water and extracted with EtOAc. The organic layer was washed with water and brine, dried over Na₂SO₄, filtered and concentrated in vacuo. The residue was purified by flash column chromatography on silica gel to afford product (2-4) (12.3 g).

To a stirred solution of compound 2-4 (6.2 g, 16.1 mmol) in THF (60 mL) at 0° C., KHDMS (1 M, 32.2 mL, 32.2 mmol) was slowly and the resulting mixture was stirred at RT for 1 h. The reaction was quenched with saturated NH₄Cl solution. The mixture was diluted with water and extracted with EtOAc. The combined organic layer was washed with brine, dried over Na₂SO₄, filtered and concentrated in vacuo. The residue was purified by flash column chromatography on silica gel to afford product (2-5) (3.9 g).

To a stirred solution of compound 2-5 (300 mg, 0.86 mmol) in acetonitrile (15 mL), POCl₃ (264 mg, 1.72 mmol) and DIEA (333 mg, 2.58 mmol) were added and the resulting mixture was stirred at 90° C. for 2 h. The mixture was concentrated in vacuo. The residue was dissolved in acetonitrile, DIEA and tert-butyl 6,6-difluoro-1,4-diazepane-1-carboxylate (223 mg, 0.95 mmol) were added. The mixture was stirred at RT for 16 h, diluted with water and extracted with EtOAc. The combined organic layer was washed with water and brine, dried over Na₂SO₄, filtered and concentrated in vacuo. The residue was purified by flash column chromatography on silica gel to afford product (2-6) (250 mg).

The mixture of compound 2-6 (150 mg, 0.26 mmol), (2-fluoro-6-hydroxyphenyl)boronic acid (50 mg, 0.32 mmol), sodium carbonate (83 mg, 0.78 mmol), Pd(PPh₃)₄ (60 mg, 0.052 mmol) in dioxane and H₂O (10 mL/2 mL) was stirred at 80° ° C. under argon for 4 h. The mixture was concentrated in vacuo. The residue was purified by flash column chromatography on silica gel to afford product (2-7) (76 mg).

To a stirred solution of compound 2-7 (150 mg, 0.23 mmol) in DCM (6 mL), TFA (2 mL) was added and the resulting mixture was stirred at RT for 1 h. The mixture was concentrated in vacuo. The residue was dissolved in DCM and treated with NH₃ in MeOH (7 N, 1 mL). The mixture was concentrated in vacuo and the residue was purified by prep-TLC plate to afford product (Compound 195) (75 mg). ESI-MS m/z: 543.4 [M+H]⁺; 1HNMR (400 MHz, DMSO-d6): δ 10.23 (s, 1H), 8.39 (d, J=5.2 Hz, 1H), 8.28 (d, J=9.2 Hz, 1H), 7.27 (m, 1H), 7.19 (d, J=5.2 Hz, 1H), 6.73 (d, J=8.4 Hz, 1H), 6.68 (t, J=8.8 Hz, 1H), 4.68 (m, 1H), 4.52 (m, 1H), 4.06-3.94 (m, 2H), 3.28 (m, 4H), 2.99 (s, 1H), 2.70 (m, 1H), 1.92 (s, 3H), 1.08 (d, J=6.8 Hz, 3H), 0.94 (d, J=6.8 Hz, 3H).

### Example 1c: Synthesis of 4-(4-(3,6-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (Compound 201)

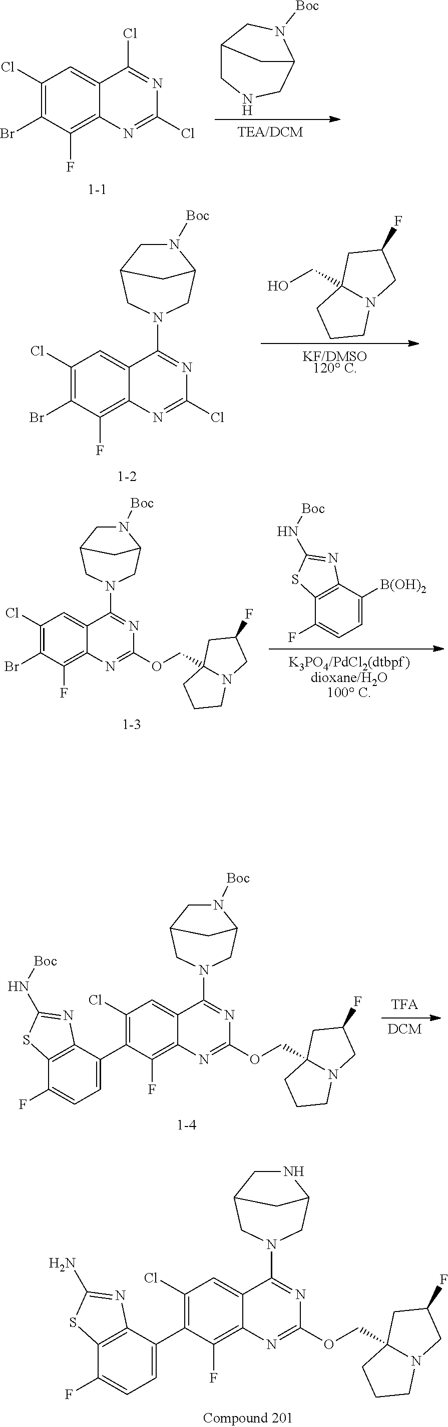

To a solution of 7-bromo-2,4,6-trichloro-8-fluoroquinazoline (1-1) (200 mg, 0.61 mmol, 1 eq) in dichloromethane (10 mL) at 0° C. were added triethylamine (184 mg, 1.83 mmol, 3 eq) and tert-butyl 3,6-diazabicyclo[3.2.1]octane-6-carboxylate (129 mg, 0.61 mmol, 1 eq). The resulting mixture was stirred at RT for 2 h. The mixture was partitioned between dichloromethane and water. The organic layer was concentrated in vacuo and the residue was purified on a silica gel column to afford compound 1-2 (300 mg). ESI-MS m/z: 506.00 [M+H]⁺.

To a solution of compound 1-2 (300 mg, 0.59 mmol) in DMSO (3 mL) were added KF (275 mg, 4.75 mmol, 8 eq) and ((2R,7aS)-2-fluorotetrahydro-1H-pyrrolizin-7a(5H)-yl)methanol (283 mg, 1.78 mmol, 3 eq). The resulting mixture was stirred at 120° C. under argon for 3 h. The mixture was partitioned between ethyl acetate and water. The organic layer was concentrated in vacuo. The residue was purified on a silica gel column to afford compound 1-3 (162 mg). ESI-MS m/z: 628.12 [M+H]⁺.

A mixture of compound 1-3 (162 mg, 0.25 mmol), (2-((tert-butoxycarbonyl)amino)-7-fluorobenzo[d]thiazol-4-yl)boronic acid (76 mg, 0.24 mmol), K₃PO₄ (77 mg, 0.36 mmol), PdCl₂(dtbpf) (12 mg, 0.018 mmol) in dioxane (6 mL) and water (1.2 mL) was stirred at 100° ° C. under argon for 2.5 h. The mixture was diluted with water (20 mL) and extracted with ethyl acetate (15 mL×2). The combined organic layer was washed with water and brine, dried over anhydrous Na₂SO₄, filtered and concentrated in vacuo. The residue was purified by prep-TLC plate to afford compound 1-4 (99 mg). ESI-MS m/z: 817.1 [M+H]⁺.

To a stirred solution of compound 1-4 (99 mg, 0.12 mmol) in dichloromethane (6 mL), was added trifluoroacetic acid (3 mL). The resulting mixture was stirred at RT for 1 h. The mixture was concentrated in vacuo. The residue was dissolved in DCM and treated with NH₃ in MeOH (7 N, 1 mL). The mixture was concentrated in vacuo and the residue was purified by prep-TLC plate to afford Compound 201 (18 mg). ESI-MS m/z: 616.4 [M+H]⁺.

### Example 1d: Synthesis of 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)naphthalen-2-ol (Compound 202)

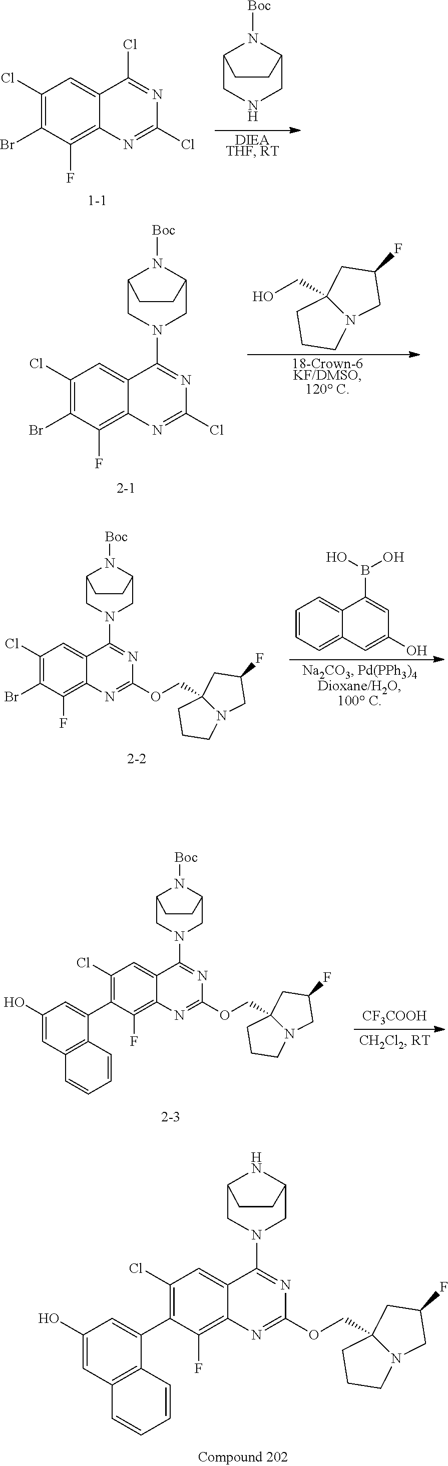

To a stirred solution of 7-bromo-2,4,6-trichloro-8-fluoroquinazoline (1-1) (323 mg, 0.99 mmol) in THF (10 mL) were added DIPEA (382 mg, 2.96 mmol) and tert-butyl-3,8-diazabicyclo[3.2.1]octane-8-carboxylate (230 mg, 1.08 mmol). The resulting mixture was stirred at RT for 1 h. The mixture was diluted with water and extracted with ethyl acetate (25 mL×2). The combined organic layer was washed with water and brine, dried over anhydrous Na₂SO₄, and concentrated in vacuo. The residue was purified by silica gel column to afford compound 2-1 (540 mg). ESI-MS m/z: 504.00 [M+H]⁺.

To a stirred solution of 2-1 (540 mg, 1.07 mmol) in DMSO (10 mL) were added ((2R,7aS)-2-fluorotetrahydro-1H-pyrrolizin-7a(5H)-yl)methanol (204 mg, 1.28 mmol), KF (1.43 g, 24.7 mmol), and 18-crown-6 (283 mg, 1.07 mmol). The resulting mixture was stirred at 120° C. under argon for 4 h. The mixture was diluted with water (50 mL) and extracted with ethyl acetate (50 mL×2). The combined organic layer was washed with water and brine and concentrated in vacuo. The residue was purified by silica gel column to afford compound 2-2 (240 mg). ESI-MS m/z: 628 [M+H]⁺.

A mixture of compound 2-2 (130 mg, 0.2 mmol), (3-hydroxynaphthalen-1-yl)boronic acid (78 mg, 0.4 mmol), Na₂CO₃ (64 mg, 0.6 mmol), Pd(PPh₃)₄ (46 mg, 0.04 mmol) in dioxane (8 mL) and water (2 mL) was stirred at 100° C. under argon for 3 h. The mixture was diluted with water (20 mL) and extracted with ethyl acetate (15 mL×2). The combined organic layer was washed with water and brine, dried over anhydrous Na₂SO₄ and concentrated in vacuo. The residue was purified by prep-TLC plate to afford the compound 2-3 (80 mg). ESI-MS m/z: 692 [M+H]⁺.

To a stirred solution of compound 2-3 (80 mg, 0.12 mmol) in dichloromethane (8 mL) was added trifluoroacetic acid (3 mL) and the resulting mixture was stirred at RT for 1 h. The mixture was concentrated in vacuo to remove the solvent. The residue was dissolved in dichloromethane and treated with NH₃ in MeOH (7 N, 1 mL) and then concentrated in vacuo. The residue was purified by prep-HPLC to afford Compound 202 (15 mg). ESI-MS m/z: 592.6 [M+H]⁺; 1HNMR (400 MHZ, DMSO-d6): δ 8.22 (s, 1H), 7.94 (s, 1H), 7.80 (d, J=8.4 Hz, 1H), 7.43 (m, 1H), 7.28 (s, 1H), 7.21 (s, 2H), 7.05 (s, 1H), 5.34 and 5.21 (s, s, 1H), 4.35 (m, 2H), 4.11-3.98 (m, 2H), 3.66-3.55 (m, 7H), 3.09-3.00 (m, 3H), 2.81 (m, 1H), 2.13-1.99 (m, 3H), 1.85-1.71 (m, 4H).

### Example 1e: Synthesis of 4-(4-(8-oxa-3-azabicyclo[3.2.1]octan-3-yl)-6-chloro-8-fluoro-2-(((2R,7aS)-2-fluorohexahydro-1H-pyrrolizin-7a-yl)methoxy)quinazolin-7-yl)-7-fluorobenzo[d]thiazol-2-amine (Compound 203)

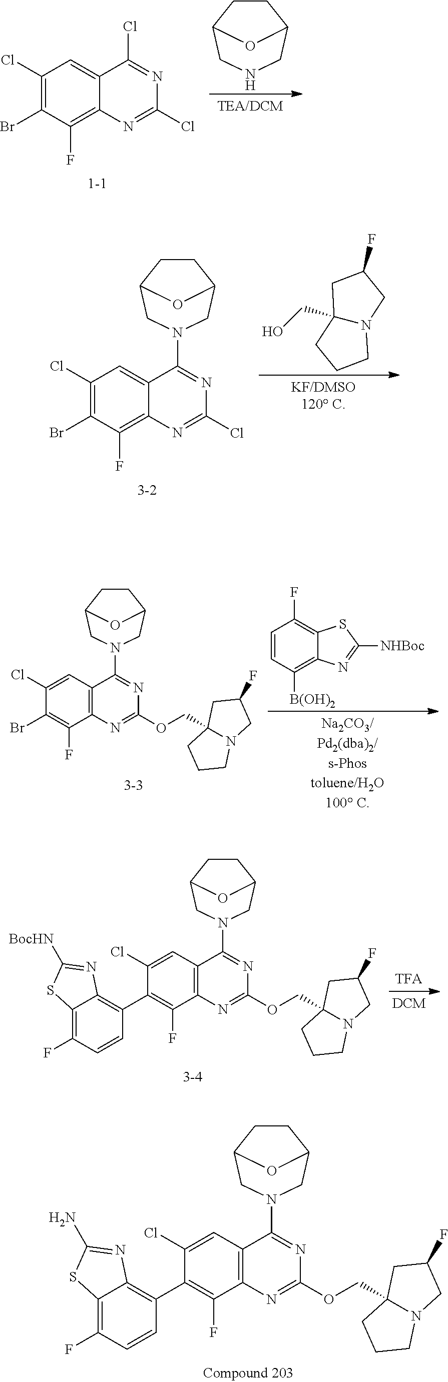

To a solution of 7-bromo-2,4,6-trichloro-8-fluoroquinazoline (1-1) (320 mg, 0.98 mmol, 1 eq) in dichloromethane (100 mL) at RT were added triethylamine (493 mg, 4.88 mmol, 5 eq) and 8-oxa-3-azabicyclo[3.2.1]octane (175 mg, 1.17 mmol, 1.2 eq). The resulting mixture was stirred at RT for 2 h. The mixture was partitioned between dichloromethane and water. The organic layer was concentrated in vacuo and the residue was purified on a silica gel column to afford compound 3-2 (300 mg). ESI-MS m/z: 408.07 [M+H]⁺.

To a solution of compound 3-2 (300 mg, 0.74 mmol) in DMSO (6 mL), were added KF (343 mg, 5.90 mmol, 8 eq) and ((2R,7aS)-2-fluorohexahydro-1H-pyrrolizin-7a-yl)methanol (176 mg, 1.11 mmol, 1.5 eq). The resulting mixture was stirred at 110° C. under argon overnight. The mixture was cooled to room temperature and partitioned between ethyl acetate and water. The organic layer was concentrated. The residue was purified on a silica gel column to afford compound 3-3 (150 mg). ESI-MS m/z: 530.81 [M+H]⁺.

A mixture of compound 3-3 (100 mg, 0.19 mmol), (2-((tert-butoxycarbonyl)amino)-7-fluorobenzo[d]thiazol-4-yl)boronic acid (88 mg, 0.28 mmol, 1.5 eq), Na₂CO₃ (60 mg, 0.57 mmol, 3 eq), Pd₂(dba)₂ (17 mg, 0.019 mmol, 0.1 eq), s-Phos (15 mg, 0.038 mmol, 0.2 eq) in toluene (10 mL) and water (1.5 mL) was stirred at 100° C. under argon overnight. The resulting mixture was cooled to room temperature and diluted with water (20 mL) and extracted with ethyl acetate (15 mL×2). The combined organic layer was washed with water and brine, dried over anhydrous Na₂SO₄, filtered and concentrated in vacuo. The residue was purified by prep-TLC plate to afford compound 3-4 (30 mg).

To a stirred solution of compound 3-4 (30 mg, 0.042 mmol) in DCM (6 mL) was added trifluoroacetic acid (2 mL) and the resulting mixture was stirred at RT for 2 h. The mixture was concentrated in vacuo. The residue was dissolved in DCM and treated with NH₃ in MeOH (7 N, 1 mL). The mixture was concentrated in vacuo and the residue was purified by prep-TLC plate to afford Compound 203 (6.5 mg). ESI-MS m/z: 618.08 [M+H]⁺; 1HNMR (400 MHZ, CD₃OD): δ 7.83 (s, 1H), 7.11 (m, 1H), 6.89 (m, 1H), 5.51 (m, 1H), 4.53 (m, 2H), 4.38-4.30 (m, 4H), 3.81-3.59 (m, 6H), 2.56-2.45 (m, 2H), 2.41-2.17 (m, 3H), 2.09-2.03 (m, 1H), 1.85-1.70 (m, 5H).

### Example 1f: Synthesis of (1R,5S)-8-(7-(2-amino-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-8-azabicyclo[3.2.1]octan-3-ol

### (Compound 307)

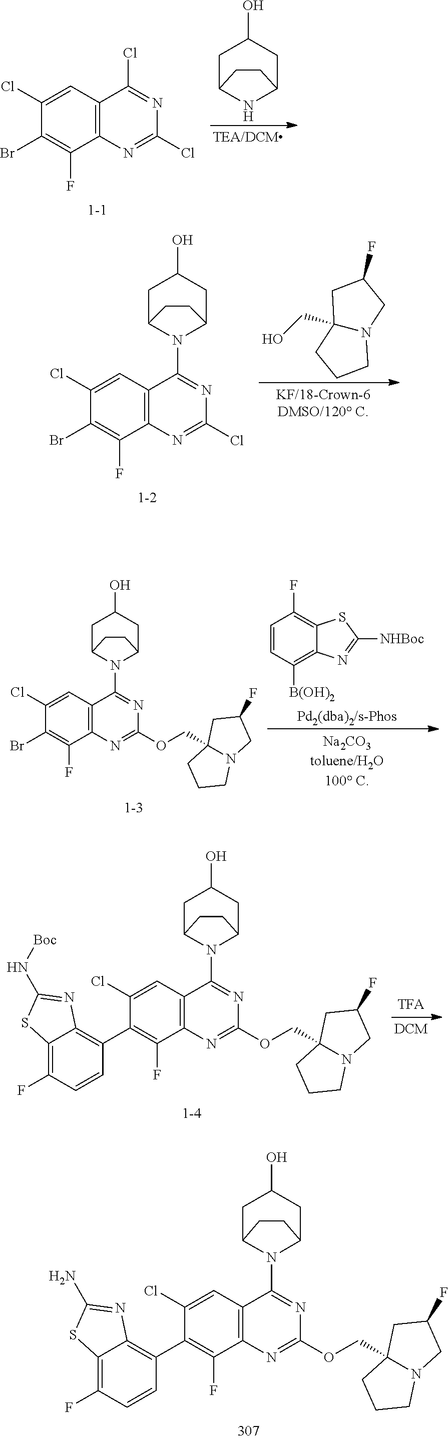

To a solution of 7-bromo-2,4,6-trichloro-8-fluoroquinazoline (1-1) (440 mg, 1.35 mmol) in dichloromethane (20 mL) at room temperature, TEA (678 mg, 6.72 mmol) and (1R,5S)-8-azabicyclo[3.2.1]octan-3-ol (176 mg, 1.37 mmol, 1.02 eq) were added and the resulting mixture was stirred at room temperature for 2 h. The mixture was partitioned between dichloromethane and water. The organic layer was concentrated in vacuo. The residue was purified on a silica gel column eluting with ethyl acetate/petroleum (1:2) to afford compound 1-2 (520 mg). ESI-MS m/z: 422.1 [M+H]⁺.

To a stirred solution of compound 1-2 (520 mg, 1.23 mmol) in DMSO (4 mL) under argon, KF (714 mg, 12.3 mmol), 18-Crown-6 (487 mg, 1.84 mmol) and ((2R,7aS)-2-fluorohexahydro-1H-pyrrolizin-7a-yl)methanol (391 mg, 2.46 mmol) were added and the resulting mixture was stirred at 120° C. for 3 h. The mixture was partitioned between ethyl acetate and water. The organic layer was concentrated in vacuo. The residue was purified on a silica gel column to afford compound 1-3 (120 mg). ESI-MS m/z: 545.2 [M+H]⁺.

The mixture of compound 1-3 (60 mg, 0.11 mmol), (2-((tert-butoxycarbonyl)amino)-7-fluorobenzo[d]thiazol-4-yl)boronic acid (69 mg, 0.22 mmol), Na₂CO₃ (41 mg, 0.39 mmol), Pd₂(dba) 3 (16 mg, 0.016 mmol) and S-phos (7 mg, 0.016 mmol) in toluene (10 mL) and water (1.5 mL) was stirred at 100° C. under argon overnight. The mixture was allowed to cool to room temperature, diluted with water (20 mL) and extracted with EtOAc (15 mL×2). The combined organic layer was washed with water and brine, dried over anhydrous Na₂SO₄, filtered and concentrated in vacuo. The residue was purified by prep-TLC plate to afford compound 1-4 (25 mg). ESI-MS m/z: 731.2 [M+H]⁺.

To a stirred solution of compound 1-4 (25 mg, 0.034 mmol) in dichloromethane (6 mL), trifluoroacetic acid (3 mL) was added and the resulting mixture was stirred at room temperature for 2 h. The mixture was concentrated in vacuo. The residue was dissolved in DCM and treated with NH₃ in MeOH (7 N, 1 mL). The mixture was concentrated in vacuo and the residue was purified by prep-TLC plate to afford compound 307 (6 mg). ESI-MS m/z: 631.4 [M+H]⁺; 1HNMR (400 MHZ, CD₃OD): 7.93 (s, 1H), 7.23-7.19 (m, 1H), 7.01-6.96 (m, 1H), 5.50-5.37 (m, 1H), 5.11-5.07 (m, 2H), 4.52-4.41 (m, 2H), 4.18-4.17 (m, 1H), 3.72-3.57 (m, 3H), 3.27-3.24 (m, 1H), 2.46-2.28 (m, 7H), 2.21-2.14 (m, 2H), 2.04-2.00 (m, 5H).

### Example 1g: Synthesis of 4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)quinazolin-7-yl)naphthalen-2-ol (Compound 308)

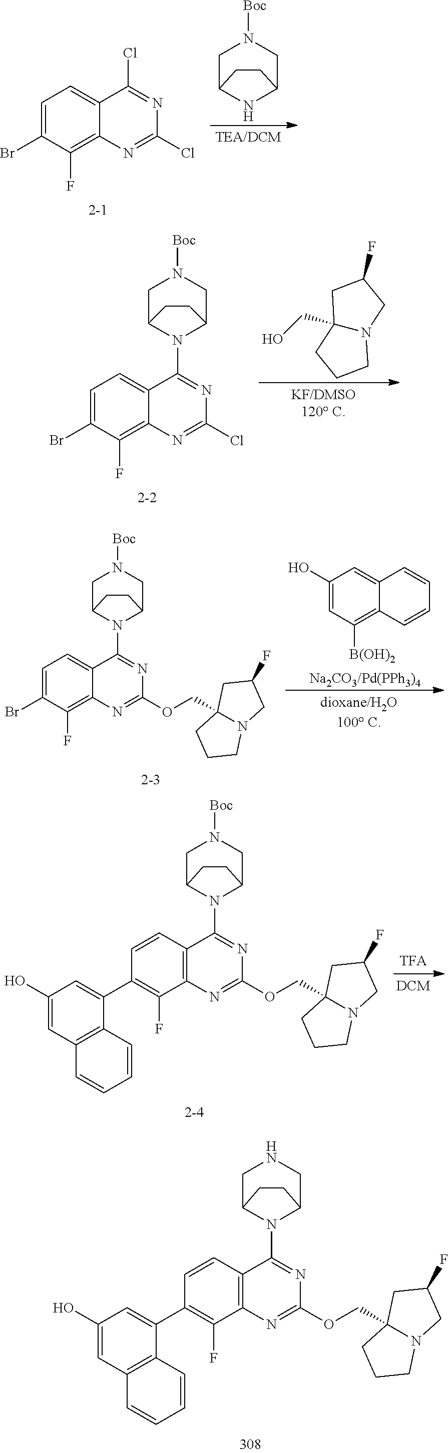

To a solution of 7-bromo-2,4-dichloro-8-fluoroquinazoline (2-1) (195 mg, 0.67 mmol) in dichloromethane (100 mL) at room temperature, TEA (339 mg, 3.36 mmol) and tert-butyl (1R,5S)-3,8-diazabicyclo[3.2.1]octane-3-carboxylate (160 mg, 0.81 mmol) were added and the resulting mixture was stirred at room temperature for 2 h. The mixture was partitioned between dichloromethane and water. The organic layer was concentrated in vacuo and the residue was purified on a silica gel column eluting with ethyl acetate/petroleum (1:4) to afford compound 2-2 (220 mg). ESI-MS m/z: 470.1 [M+H]⁺.

To a solution of compound 2-2 (240 mg, 0.52 mmol) in DMSO (3 mL) under argon, KF (243 mg, 4.19 mmol) and ((2R,7aS)-2-fluorohexahydro-1H-pyrrolizin-7a-yl)methanol (125 mg, 0.79 mmol) were added and the resulting mixture was stirred at 120° C. for 3 h. The mixture was partitioned between ethyl acetate and water. The organic layer was concentrated in vacuo. The residue was purified on a silica gel column to afford compound 2-3 (100 mg). ESI-MS m/z: 595.2 [M+H]⁺.

The mixture of compound 2-3 (100 mg, 0.17 mmol), (3-hydroxynaphthalen-1-yl)boronic acid (60 mg, 0.34 mmol), Na₂CO₃ (137 mg, 0.64 mmol), Pd(PPh3)₄ (20 mg, 0.017 mmol) in 1,4-dioxane (15 mL) and water (3 mL) was stirred at 100° C. under argon for 3 h. The mixture was diluted with water (20 mL) and extracted with EtOAc (15 mL×2). The combined organic layer was washed with water and brine, dried over anhydrous Na₂SO₄, filtered and concentrated in vacuo. The residue was purified by prep-TLC plate to afford compound 2-4 (60 mg).

To a stirred solution of compound 2-4 (60 mg, 0.1 mmol) in DCM (6 mL), trifluoroacetic acid (2 mL) was added and the resulting mixture was stirred at room temperature for 1 h. The mixture was concentrated in vacuo. The residue was dissolved in DCM and treated with NH₃ in MeOH (7 N, 1 mL). The mixture was concentrated in vacuo and the residue was purified by prep-TLC plate to afford the compound 308 (25 mg). ESI-MS m/z: 558.3 [M+H]⁺; 1HNMR (400 MHZ, DMSO-d6): 10.14 (s, 1H), 8.03 (d, J=8.4 Hz, 1H), 7.81 (d, J=8.4 Hz, 1H), 7.45 (t, J=6.8 Hz, 1H), 7.37 (m, 2H), 7.29 (s, 1H), 7.25 (t, J=7.2 Hz, 1H), 7.15 (d, J=2.0 Hz, 1H), 5.45 (s, s, 1H), 5.05 (s, 2H), 4.37 (m, 2H), 3.43 (m, 4H), 3.28 (m, 3H), 3.07 (m, 1H), 2.28 (m, 2H), 2.16 (m, 5H), 1.99 (m, 3H).

### Example 1h: Synthesis of (S)-4-(4-(6,6-difluoro-1,4-diazepan-1-yl)-2-((1-methylpyrrolidin-2-yl)methoxy)-5,8-dihydropyrido[3,4-d]pyrimidin-7(6H)-yl)naphthalen-2-ol (Compound 419)

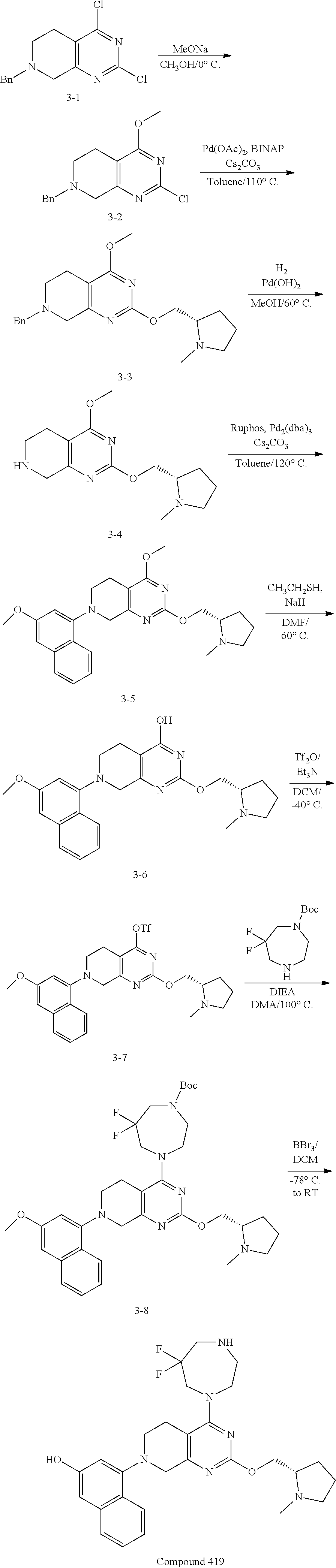

To a solution of 7-benzyl-2,4-dichloro-5,6,7,8-tetrahydropyrido[3,4-d]pyrimidine (3-1) (5 g, 16.9 mmol) in MeOH (50 mL) at ° C., NaOCH₃ (1.37 g, 25.49 mmol) was added in portions and the resulting mixture was stirred at ° C. for 2 h. The reaction was quenched with saturated NH₄Cl aqueous solution, and extracted with DCM. The organic layer was washed with water and brine, dried over Na₂SO₄, filtered and concentrated in vacuo. The residue was purified by flash chromatography on silica gel to afford product (3-2) (4.7 g).

The mixture of compound 3-2 (4.7 g, 16.22 mmol), (S)-(1-methylpyrrolidin-2-yl)methanol (3.7 g, 32.44 mmol), Pd(OAc)₂ (363 mg, 1.62 mmol), BINAP (2 g, 3.24 mmol), Cs₂CO₃ (15.9 g, 48.66 mmol) in toluene (100 mL) was stirred at 110° ° C. under argon for 16 h. The mixture was filtered through celite. The filtrate was diluted with water and EtOAc, and acidified with 2N HCl aqueous solution to adjust the pH to 3-4. The organic layer was separated, and the water layer was basified with Na₂CO₃ solid to adjust the pH to 8-9. The mixture was extracted with EtOAc. The organic layer was washed with water and brine, dried over Na₂SO₄, filtered and concentrated in vacuo. The residue was purified by flash column chromatography on silica gel to afford product (3-3) (3.74 g).

Compound 3-3 (3.74 g, 10.16 mmol) was dissolved in MeOH (50 mL) and Pd(OH)₂/C (900 mg) was added. The mixture was stirred at 60° ° C. under hydrogen for 3 h. The mixture was allowed to cool to RT and filtered through celite. The filtrate was concentrated in vacuo to afford product (3-4) (2.8 g).

The mixture of compound 3-4 (1.7 g, 6.1 mmol), 3-methoxynaphthalen-1-yl trifluoromethanesulfonate (2.8 g, 9.15 mmol), Ruphos (569 mg, 1.22 mmol), Pd₂(dba)₃ (1.16 g, 1.22 mmol), Cs₂CO₃ (6 g, 18.3 mmol) in toluene (80 mL) was stirred at 120° ° C. under argon for 16 h. The mixture was allowed to cool to RT and concentrated in vacuo. The residue was purified by flash column chromatography on silica gel to afford product (3-5) (800 mg).

To a stirred mixture of NaH (184 mg, 4.6 mmol) in DMF (10 mL) at 0° C., ethyl mercaptan (485 mg, 7.82 mmol) was slowly and the resulting mixture was stirred for 30 min. A solution of compound 3-5 (800 mg, 1.84 mmol) in DMF (10 mL) was added to the mixture and then stirred at 60° C. for 3 h. The reaction was quenched with saturated NH₄Cl aqueous solution, and extracted with EtOAc. The organic layer was washed with water and brine, dried over Na₂SO₄, filtered and concentrated in vacuo. The residue was purified by flash column chromatography on silica gel to afford product (3-6) (382 mg).

To a stirred solution of compound 3-6 (380 mg, 0.9 mmol) in DCM (10 mL) at −40° C., TEA (273 mg, 2.7 mmol) was added followed by Tf₂O (509 mg, 1.8 mmol). The resulting mixture was stirred at −40° C. for 1.5 h. The reaction was quenched with saturated NH₄Cl aqueous solution, and extracted with DCM. The organic layer was washed with water and brine, dried over Na₂SO₄, filtered and concentrated in vacuo. The residue was purified by flash column chromatography on silica gel to afford product (3-7) (160 mg).

The mixture of compound 3-7 (400 mg, 0.72 mmol), tert-butyl 6,6-difluoro-1,4-diazepane-1-carboxylate (256 mg, 1.08 mmol), DIEA (280 mg, 2.17 mmol) in DMA (10 mL) in seal tube was stirred at 100° ° C. for 6 h. The mixture was poured into water and extracted with EtOAc. The organic layer was washed with water and brine, dried over Na₂SO₄, filtered and concentrated in vacuo. The residue was purified by flash column chromatography on silica gel to afford product (3-8) (200 mg).

To a stirred solution of compound 3-8 (100 mg, 0.16 mmol) in DCM (10 mL) at −78° C., BBr₃ (196 mg, 0.78 mmol) was added and the resulting mixture was stirred at RT for 2 h. The mixture was cooled to −78° C., quenched with water. And basified with NH₃ in MeOH (7 N) to adjust the pH to 8-9. The mixture was extracted with DCM, washed with water and brine, dried over Na₂SO₄, filtered and concentrated in vacuo. The residue was purified by prep-HPLC to afford compound 419 as a formic acid salt (16 mg). ESI-MS m/z: 525.45 [M+H]⁺; ¹HNMR (400 MHZ, DMSO-d6): δ 8.21 (s, 1H), 8.14 (d, J=16.8 Hz, 1H), 7.98 (m, 1H), 7.66 (d, J=8.0 Hz, 1H), 7.39 (t, J=6.8 Hz, 1H), 7.28 (t, J=7.2 Hz, 1H), 6.86 (s, 1H), 6.76 (s, 1H), 4.30 (m, 2H), 4.13 (m, 2H), 3.82 (m, 3H), 3.28 (m, 2H), 3.03 (m, 2H), 2.92 (s, 2H), 2.69 (m, 1H), 2.42 (s, 3H), 2.28 (m, 1H), 1.99 (m, 1H), 1.729-1.611 (m, 3H).

### Example 1i: Synthesis of 4-(4-((1R,5S)-3,8-diazabicyclo[3.2.1]octan-3-yl)-6-methoxy-2-((tetrahydro-1H-pyrrolizin-7a(5H)-yl)methoxy)pyrido[3,2-d]pyrimidin-7-yl)naphthalen-2-ol (Compound 545)

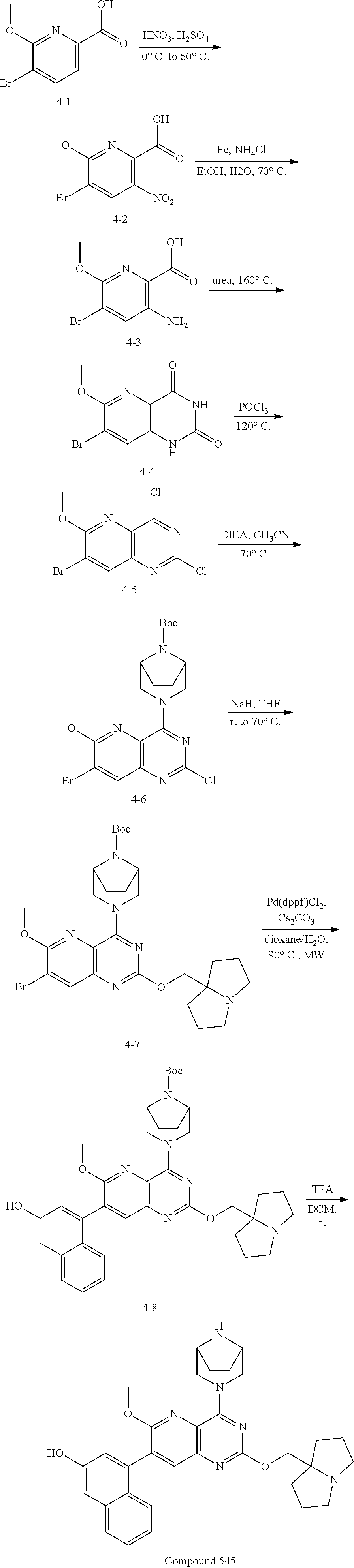

At 0° C., to a suspension of compound 4-1 (3.0 g, 12.9 mmol) in conc·H₂SO₄ (10 ml) was added dropwise fuming HNO₃ (4.06 g, 64.5 mmol), the mixture was stirred for 16 h at 60° C., after cooling, the mixture was poured into crash ice, extracted with EA (100 ml*3), the combined extracts were washed with brine, dried over anhydrous Na₂SO₄, concentrated in vacuo, the residue was purified by flash column chromatography on silica gel (eluting with 0˜100% of EA in PE) to give compound 4-2 (2.0 g). ESI-MS m/z: 277.0 [M+H]⁺.

To a solution of compound 4-2 (2.0 g, 7.22 mmol) in EtOH/H₂O (50 ml/10 ml) was added Fe powder (2.02 g, 36.1 mmol), NH₄Cl (3.86 g, 72.2 mmol), the mixture was stirred at 80° C. for 2 h. Removed the solvent in vacuo, the residue was purified by flash column chromatography on silica gel (eluting with 0˜20% of MeOH in DCM) to give compound 4-3 (560 mg). ESI-MS m/z: 246.9 [M+H]⁺.

A mixture of compound 4-3 (3.4 g, 13.8 mmol) and urea (10.2 g) was heated and stirred for 5 h at 160° C. After cooling, H₂O (100 ml) was added and stirred for 30 min, the precipitate was collected by filtration to give compound 4-4 (2.8 g). ESI-MS m/z: 274.0 [M+H]⁺.

Compound 4-4 (6.0 g, 22.1 mmol) was dissolved in POCl₃ (50 ml), to the solution was added N,N-dimethylaniline (5.36 g, 44.2 mmol), the mixture was stirred at 120° C. under Ar₂ for 30 min. Removed the solvent, the resulted residue was purified by flash column chromatography on silica gel (eluting with 0˜20% of EA in PE) to give compound 4-5 (6.20 g). ESI-MS m/z: 312.8 [M+H]⁺.

Under Ar₂, to a solution of compound 4-5 (30 mg, 0.097 mmol) in anhydrous CAN (5 ml) was added DIEA (37.6 mg, 0.29 mmol), followed by tert-butyl (1R,5S)-3,8-diazabicyclo[3.2.1]octane-8-carboxylate (22.7 mg, 0.107 mmol). The mixture was stirred for 2 h at 50° ° C. Removed the solvent in vacuo, the resulted residue was purified by flash column chromatography on silica gel (eluting with 0˜20% of EA in PE) to give compound 4-6 (20 mg). ESI-MS m/z: 485.9 [M+H]⁺.

To a solution of (tetrahydro-1H-pyrrolizin-7a(5H)-yl)methanol (52.5 mg, 0.372 mmol) in dry THF(5 ml) cooled to 0° C. with an a ice bath was added NaH (60%, 15 mg, 0.372 mmol). The mixture was stirred for 30 min, then compound 4-6 (90 mg, 0.186 mmol) was added and the mixture was stirred for 2 h at 70° C. LCMS showed the starting material was consumed. Quenched with H₂O at 0° C., the mixture was concentrated in vcauo and the residue was purified by flash column chromatography on silica gel (eluting with 0˜20% of MeOH in DCM) to give compound 4-7 (100 mg). ESI-MS m/z: 591.1 [M+H]⁺.
Under Ar2, to a solution of compound 4-7 (50 mg, 0.0848 mmol) in 1,4-dioxane/H2O (5 ml/1 ml) was added (3-hydroxynaphthalen-1-yl)boronic acid (23 mg, 0.0848 mmol), Cs₂CO₃ (55.4 mg, 0.17 mmol) and Pd(dppf)Cl₂ (10 mg). The mixture was stirred for 0.5h at 90° C. under microwave irritation. Removed the solvent in vacuo, the residue was purified by flash column chromatography on silica gel (eluting with 0˜20% of MeOH in DCM) to give compound 4-8 (38 mg). ESI-MS m/z: 653.3 [M+H]⁺.

Compound 4-8 (38 mg, 0.0582 mmol) was dissolved in anhydrous DCM (1.0 ml), TFA (0.3 ml) was added and the reaction mixture was stirred for 16 h at RT. Removed the solvent and the resulted residue was purified by Prep-HPLC to give compound 545 (12.23 mg). ESI-MS m/z: 553.4 [M+H]⁺; 1HNMR (400 MHZ, DMSO-d6): δ 8.25 (s, 2H), 7.79-7.75 (m, 2H), 7.41 (dd, J=11.0, 4.0 Hz, 1H), 7.30 (d, J=8.4 Hz, 1H), 7.20 (dd, J=11.2, 4.1 Hz, 2H), 7.04 (d, J=2.4 Hz, 1H), 4.10 (s, 2H), 3.86 (s, 2H), 3.80 (s, 3H), 3.43 (d, J=13.2 Hz, 2H), 3.10-3.03 (m, 2H), 2.70 (dt, J=10.3, 6.6 Hz, 2H), 2.00-1.75 (m, 12H), 1.71-1.61 (m, 3H).

### Example 1j: Synthesis of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)naphthalen-2-ol (Compound 610 and Compound 429)

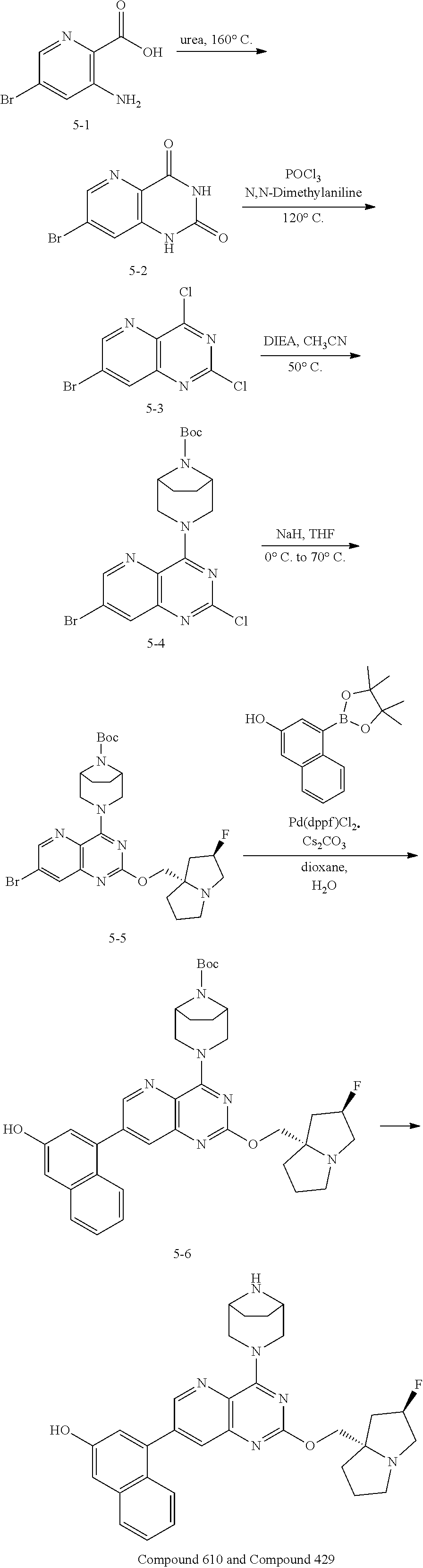

A mixture of compound 5-1 (2.0 g, 9.2 mmol) and urea (8.28 g, 138 mmol) were stirred at 160° C. for 5 h under N₂. The mixture was cooled to 80° C. and quenched by water (5 mL). The mixture was cooled to room temperature and triturated with MeOH (15 mL) and H₂O (10 mL). The residue was filtered to give compound 5-2 (1.5 g). ESI-MS m/z: 242.1 [M+H]⁺.

A solution of compound 5-2 (4.0 g, 16.5 mmol) in POCl₃ (20 mL), N,N-Dimethylaniline (4.0 g) was added. The reaction mixture was stirred at 120° ° C. for 3 h. The mixture was concentrated to remove POCl₃, and the residue was purified by flash column chromatography on silica gel (PE:EA=15:1˜3:1) to compound 5-3 (4.0 g). ESI-MS m/z: 280.1 [M+H]⁺.

A solution of compound 5-3 (250 mg, 0.902 mmol) in MeCN (10 mL), tert-butyl (1R,5S)-3,8-diazabicyclo[3.2.1]octane-8-carboxylate (213 mg, 0.993 mmol) and DIEA (350 mg, 2.706 mmol) was added. The mixture was stirred at 70° C. for 2h. The mixture was concentrated and purified by flash column chromatography on silica gel (PE:EA=50:1˜5:1) to afford compound 5-4 (300 mg). ESI-MS m/z: 456.3 [M+H]⁺.

A solution of ((2R,7aS)-2-fluorotetrahydro-1H-pyrrolizin-7a(5H)-yl)methanol (189 mg, 1.18 mmol) in THF (10 mL), NaH (45 mg, 1.18 mmol) was added. The mixture was stirred at r.t. for 30 min. Compound 5-4 (270 mg, 0.585 mmol) was added. The mixture was stirred at 70° C. for 2 h. The mixture was quenched by water (3 mL) and the residue was extracted with EA (10 mL×3). The residue was purified by flash column chromatography on silica gel (DCM:MeOH=10:1) to afford compound 5-5 (240 mg). ESI-MS m/z: 577.4 [M+H]⁺.

A solution of compound 5-5 (80 mg, 1.36 mmol) and 4-(4,4,5,5-tetramethyl-1,3,2-dioxaborolan-2-yl)naphthalen-2-ol (44 mg, 0.164 mmol) in dioxane/H₂O (3 mL/1 mL), Cs₂CO₃ (136 mg, 0.408 mmol) and Pd(dtbpf)Cl₂ (20 mg, 0.136 mmol) was added. The mixture was stirred at 90° C. for 30 min under Ar₂. Water (2 mL) was added and the residue was extracted with EA (5 mL×3). The combined organic layers were concentrated and purified by flash column chromatography on silica gel (DCM:MeOH=20:1˜10:1) to afford compound 5-6 (60 mg). ESI-MS m/z: 641.3 [M+H]⁺.

To a solution of compound 5-6 (40 mg, 0.062 mmol) in DCM (2 mL) was added HCl/dioxane (2 mL, 1M). The mixture was stirred at r.t. for 30 min. The mixture was concentrated under reduced pressure. The residue was purified by prep-HPLC (FA) to afford two atropisomers compound 610 (14.57 mg) and compound 429 (16.49 mg).

Compound 610: ESI-MS m/z: 541.5 [M+H]⁺; 1HNMR (400 MHZ, DMSO-d6): δ 8.65 (d, J=2.1 Hz, 1H), 8.21 (s, 1H), 7.90 (d, J=2.1 Hz, 1H), 7.82 (d, J=8.2 Hz, 1H), 7.62 (d, J=8.4 Hz, 1H), 7.46 (t, J=7.5 Hz, 1H), 7.32-7.23 (m, 2H), 7.13 (d, J=2.4 Hz, 1H), 5.28 (d, J=52.9 Hz, 2H), 4.09 (d, J=10.2 Hz, 1H), 3.99 (d, J=10.3 Hz, 1H), 3.69 (s, 3H), 3.09 (d, J=11.2 Hz, 3H), 3.02 (s, 2H), 2.83 (d, J=6.5 Hz, 1H), 2.20-2.09 (m, 1H), 2.03 (d, J=17.8 Hz, 2H), 1.84 (d, J=15.6 Hz, 2H), 1.76 (s, 6H).

Compound 429: ESI-MS m/z: 541.5 [M+H]⁺; 1HNMR (400 MHz, DMSO-d6): δ 8.66 (d, J=2.1 Hz, 1H), 8.23 (s, 1H), 7.92 (d, J=2.1 Hz, 1H), 7.82 (d, J=8.3 Hz, 1H), 7.62 (d, J=8.5 Hz, 1H), 7.47 (t, J=7.3 Hz, 1H), 7.29 (dd, J=9.3, 4.8 Hz, 2H), 7.14 (d, J=2.3 Hz, 1H), 5.29 (d, J=53.1 Hz, 2H), 4.10 (d, J=10.4 Hz, 1H), 4.01 (d, J=10.3 Hz, 1H), 3.83 (s, 3H), 3.17 (s, 5H), 3.13-3.07 (m, 3H), 3.03 (s, 1H), 2.84 (dd, J=14.7, 8.3 Hz, 1H), 2.20-2.11 (m, 1H), 2.05 (t, J=12.1 Hz, 2H), 1.86 (s, 1H), 1.82 (s, 4H), 1.77 (s, 1H).

### Example 1k: Synthesis of 4-(4-((1R,5S)-3,8-diazabicyclo[3.2.1]octan-3-yl)-2-((tetrahydro-1H-pyrrolizin-7a(5H)-yl)methoxy)-5,6,8,9-tetrahydro-7H-pyrimido[4,5-d]azepin-7-yl)naphthalen-2-ol (Compound 521)

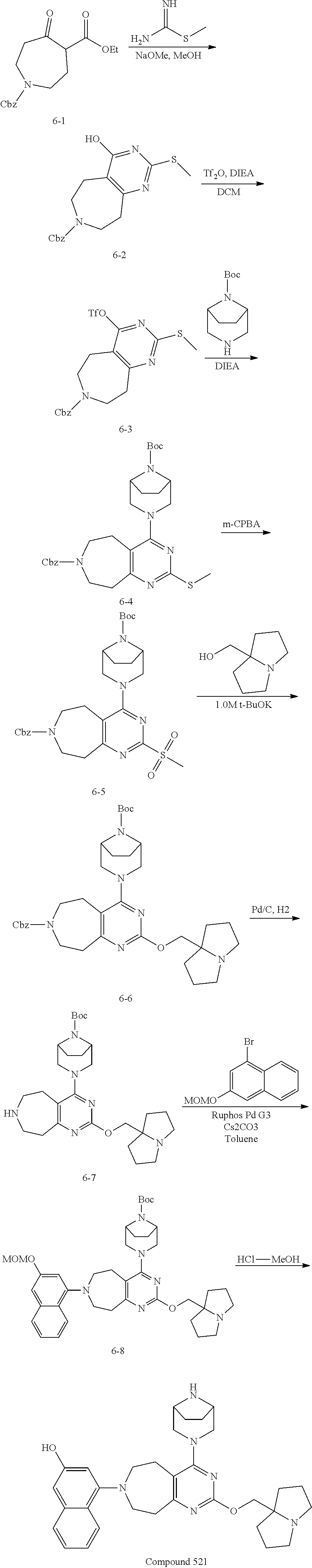

To a solution of compound 6-1 (5 g, 15.6 mmol) and methyl carbamimidothioate (2.8 g, 31.2 mmol) in 20 ml of MeOH was added NaOMe (4.3 g, 93.6 mmol) at 0° C., the mixture was stirred at 25° C. for 4 hours. This reaction was concentrated under reduced pressure. The residue was purified by flash column chromatography on silica gel using 65% ethyl acetate in hexane as eluent to afford compound 6-2 (2.5 g). ESI-MS m/z: 346.2 [M+H]⁺.

The reaction mixture of compound 6-2 (2.5 g, 7.25 mmol), Tf₂O (4 g, 14.4 mmol), DIEA (2.8 g, 21.75 mmol) in 20 mL of DCM was sealed at 0° C. and heated at 25° ° C. for 2 hours. The reaction was poured into water and extracted with ethyl acetate (3×20 mL). Combined organic layers were washed with water (2×20 mL), brine (40 mL), dried over anhydrous sodium sulfate and concentrated under reduced pressure to give compound 6-3 (2.9 g). ESI-MS m/z: 478.2 [M+H]⁺.

The reaction mixture of compound 6-3 (2.9 g, 6.1 mmol), tert-butyl (1R,5S)-3,8-diazabicyclo[3.2.1]octane-8-carboxylate (1.4 g, 6.6 mmol), DIEA (2.3 g, 18.3 mmol) in 20 mL of DMF was sealed and heated at 50° C. for 2 hours. The reaction was poured into water and extracted with ethyl acetate (3×20 mL). Combined organic layers were washed with water (2×10 mL), brine (10 mL), dried over anhydrous sodium sulfate and concentrated under reduced pressure. The residue was purified by flash column chromatography on silica gel (100-200 mesh) using 55% ethyl acetate in hexane as eluent to afford compound 6-4 (2.5 g). ESI-MS m/z: 540.4 [M+H]⁺.

To a solution of compound 6-4 (2.5 g, 4.6 mmol) in 20 ml DCM was added m-CPBA (2.5 g, 13.9 mmol) at 0° C., the mixture was stirred at 25° C. for 16 hours. The reaction was poured into Na₂SO₃ solution and extracted with Ethyl Acetate (3×20 mL). Combined organic layers were washed with Na₂CO₃ (2×20 mL), brine (20 mL), dried over anhydrous sodium sulfate and concentrated under reduced pressure to afford compound 6-5 (2.5 g). ESI-MS m/z: 572.5 [M+H]⁺.

To a solution of (tetrahydro-1H-pyrrolizin-7a(5H)-yl)methanol (676.8 mg, 4.8 mmol) in 20 mL of THF was added t-BuOK (8.3 ml, 4.8 mmol) at 0° C. The mixture was stirred at 0° C. for 0.5 hour, then compound 6-5 (2.5 g, 4.4 mmol) was added and stirred at 25° ° C. for 2 hours. The reaction was poured into water and extracted with ethyl acetate (3×20 mL). Combined organic layers were washed with water (2×20 mL), brine (20 mL), dried over anhydrous sodium sulfate and concentrated under reduced pressure. The residue was purified by flash column chromatography on silica gel to afford compound 6-6 (1.3 g). ESI-MS m/z: 633.6 [M+H]⁺.

To a solution of compound 6-6 (500 mg, 0.79 mol) in 20 mL of MeOH was added Pd/C(200 mg), the mixture was stirred under H₂ for 2 hours. The mixture was filtered and concentrated in vacuo to give compound 6-7 (300 mg). ESI-MS m/z: 499.5 [M+H]⁺.

A mixture of 6-7 (100 mg, 0.2 mmol), 1-bromo-3-(methoxymethoxy)naphthalene (106 mg, 0.4 mmol), Ruphos Pd G3 (16.7 mg, 0.02 mmol) and Cs₂CO₃ (195.6 mg, 0.6 mmol) in toluene(1.0 ml) was stirred at 80° ° C. for 16 hours. The cooled reaction mixture was diluted with EtOAc (20 mL) and washed with water (10 mL). the combined organics were washed with brine (10 mL), dried (MgSO4), filtered and concentrated in vacuo to give a yellow residue. This was purified by flash column chromatography on silica gel (DCM/MeOH=10:1) to give compound 6-8 (40 mg). ESI-MS m/z: 685.6 [M+H]⁺.

To a solution of compound 6-8 (40 mg, 0.058 mmol) in 4 ml of HCl-MeOH at 25° C., the mixture was stirred at 25° C. for 2 h. This reaction was concentrated under reduced pressure and purified by prep-HPLC to give compound 521 (5 mg). ESI-MS m/z: 541.6 [M+H]⁺.

### Example 11: Synthesis of 4-(6-chloro-4-(6,6-difluoro-1,4-diazepan-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (Compound 133)

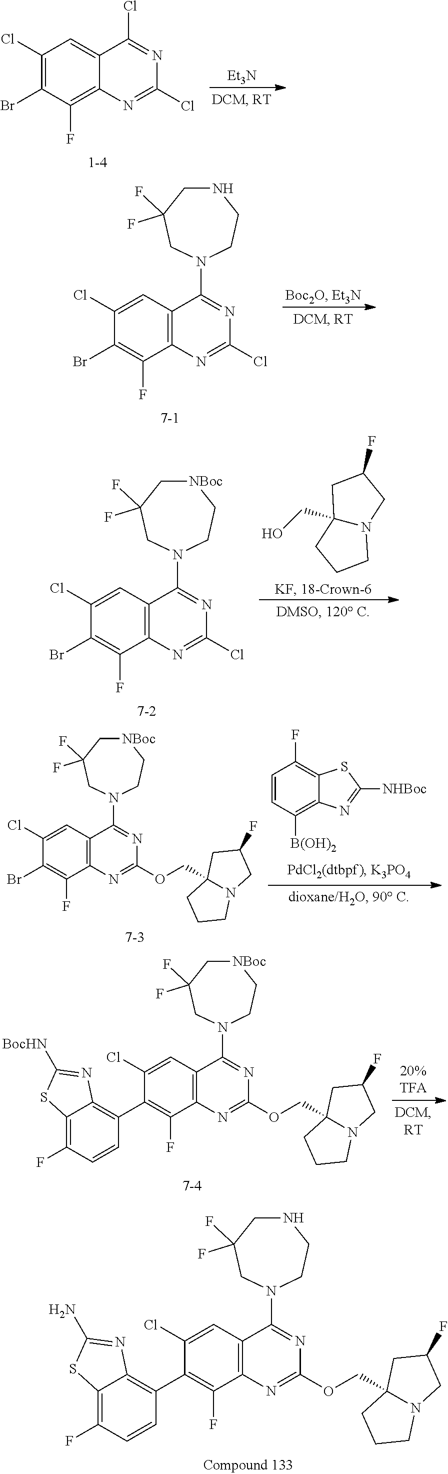

To a stirred solution of 7-bromo-2,4,6-trichloro-8-fluoroquinazoline (1-4) (1.2 g, 3.64 mmol) in DCM (20 mL) at ice-water bath was added Et₃N (1.75 mL, 12.74 mmol) followed by 6,6-difluoro-1,4-diazepane dihydrochloride (750 mg, 3.59 mmol) slowly. The reaction mixture was warmed to room temperature and stirred overnight. Diluted with water, extracted with DCM. The combined organics were washed with water, dried over anhydrous Na₂SO₄ then concentrated in vacuo. The residue was purified by silica gel column chromatography eluting with 0-100% ethyl acetate/hexane with 0.5% Et₃N) to afford compound 7-1 (1.2 g). ESI-MS m/z: 428.9 [M+H]⁺.

The reaction mixture of compound 7-1 (250 mg, 0.58 mmol), Et₃N (120 μL, 0.87 mmol), Boc₂O (190 μL, 0.87 mmol) and DMAP (15 mg, 0.12 mmol) in DCM (8 mL) was stirred at room temperature overnight. Concentrated, the residue was purified by silica gel column chromatography eluting with 0-70% ethyl acetate/hexane to afford compound 7-2 (270 mg). ESI-MS m/z: 529.0 [M+H]⁺.

To a stirred solution of compound 7-2 (270 mg, 0.51 mmol) in DMSO (8 ml) was added ((2R,7aS)-2-fluorotetrahydro-1H-pyrrolizin-7a(5H)-yl)methanol (185 mg, 1.17 mmol), potassium fluoride (148 mg, 2.55 mmol) and 18-crown-6 (175 mg, 0.66 mmol). The reaction mixture was heated at 120° C. for 2 h under nitrogen. The reaction mixture was cooled down, diluted with water, extracted with ethyl acetate. The combined organic layers were washed with water, brine and concentrated. The residue was purified by silica gel column chromatography eluting with 0-90% ethyl acetate/hexane with 0.5% Et₃N to afford compound 7-3 (155 mg). ESI-MS m/z: 652.1 [M+H]⁺.

The reaction mixture of compound 7-3 (70 mg, 0.108 mmol), (2-((tert-butoxycarbonyl)amino)-7-fluorobenzo[d]thiazol-4-yl)boronic acid (64 mg, 0.16 mmol), K₃PO₄ (41 mg, 0.19 mmol) and dichloro[1,1′-bis(di-tert-butylphosphino)ferrocene]palladium (II) (14 mg, 0.025 mmol) in a mixed solvent of dioxane-water (1.6 mL, 3:1) was heated at 90° ° C. for 2 h under nitrogen. Filtered through Celite, the filtrate was concentrated, the residue was purified by silica gel column chromatography eluting with 0-100% ethyl acetate/hexane with 0.5% Et₃N to afford compound 7-4 (65 mg). ESI-MS m/z: 840.2 [M+H]⁺.

The reaction mixture of compound 7-4 (65 mg, 0.077 mmol) in 20% TFA in DCM (5 mL) was stirred at room temperature for 4 h, then concentrated. The residue was purified by HPLC (5-60% CH₃CN/H₂O with 0.1% formic acid), the combined fractions were neutralized with saturated aqueous sodium bicarbonate, extracted with DCM. The combined fractions were washed with water, dried over anhydrous Na₂SO₄, filtered then concentrated to afford compound 133 (33 mg). ESI-MS m/z: 640.2 [M+H]⁺; 1HNMR (400 MHZ, DMSO-d6): 7.95 (s, 1H), 7.92 (s, 2H), 7.24 (dd, J=8.4 and 5.6 Hz, 1H), 7.07 (t, J=8.8 Hz, 1H), 5.35-5.19 (m, 1H), 4.63-4.38 (m, 2H), 4.16-3.98 (m, 2H), 3.95-3.85 (m, 2H), 3.28-3.15 (m, 4H), 3.12-2.95 (m, 4H), 2.84-2.78 (m, 1H), 2.18-1.95 (m, 3H), 1.90-1.75 (m, 3H).

### Example 1m: Synthesis of 4-(7-(2-amino-7-fluorobenzo[d]thiazol-4-yl)-6-chloro-8-fluoro-2-((tetrahydro-1H-pyrrolizin-7a(5H)-yl)methoxy)quinazolin-4-yl)-6,6-difluoro-1,4-diazepane-1-carbonitrile (Compound 551)

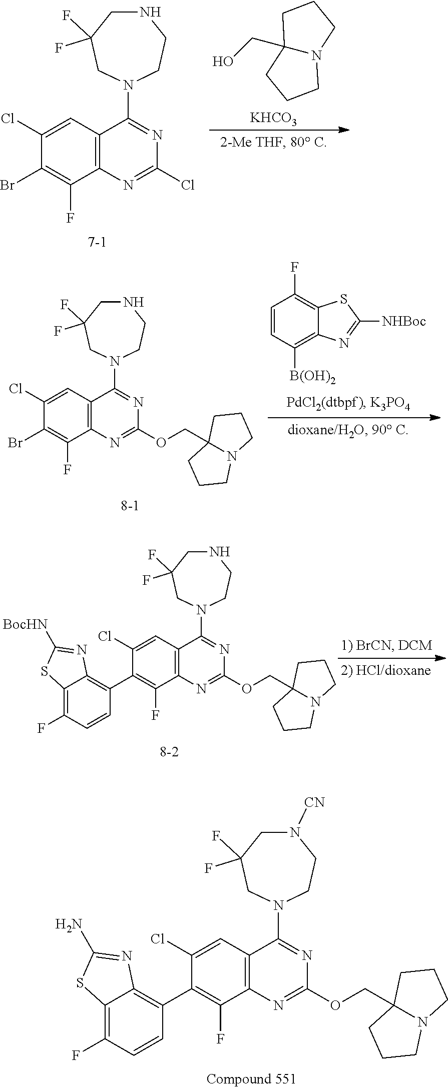

To a stirred solution of compound 7-1 (660 mg, 1.5 mmol) in 2-methyl tetrahydrofuran (10 mL) was added (tetrahydro-1H-pyrrolizin-7a(5H)-yl)methanol (495 mg, 3.5 mmol) and potassium bicarbonate (900 mg, 3.5 mmol). The reaction mixture was heated at 80° C. for 2 days. The solid was filtered off, washed with ethyl acetate. The filtrate was concentrated, the residue was purified by reverse phase column (5-60% CH₃CN/H₂O with 0.1% TFA). The combined fractions were neutralized with saturated aqueous sodium bicarbonate, extracted with DCM. The combined organic layers were dried over anhydrous Na₂SO₄, filtered then concentrated to afford compound 8-1. ESI-MS m/z: 534.0 [M+H]⁺.

The reaction mixture of compound 8-1 (35 mg, 0.058 mmol), (2-((tert-butoxycarbonyl)amino)-7-fluorobenzo[d]thiazol-4-yl)boronic acid (27 mg, 0.087 mmol), K₃PO₄ (22 mg, 0.1 mmol) and dichloro[1,1′-bis(di-tert-butylphosphino)ferrocene]palladium (II) (7 mg, 0.012 mmol) in a mixed solvent of dioxane-water (1.6 mL, 3:1) was heated at 90° C. for 2 h under nitrogen. Filtered through Celite, the filtrate was concentrated, the residue was purified by silica gel column chromatography further by HPLC to afford compound 8-2 (8 mg). ESI-MS m/z: 722.2 [M+H]⁺.

To a stirred solution of compound 8-2 (8 mg, 0.011 mmol) in DCM (1 mL) was added Et₃N (3 μL, 0.022 mmol) followed by a 1 M solution of BrCN in DCM (20 μL, 0.02 mmol). The reaction mixture was stirred at RT overnight. Diluted with aqueous sodium bicarbonate, extracted with DCM. The combined organics were washed with water then concentrated. The residue was dissolved in dioxane (0.5 mL), a 4 M solution of HCl in dioxane (0.5 mL) was added. The reaction mixture was stirred at room temperature for 2 h then concentrated. The residue was purified by HPLC (5-60% CH₃CN/H₂O with 0.1% formic acid), the combined fractions were lyophilized to afford compound 551 (1 mg) as formic acid salt. ESI-MS m/z: 647.2 [M+H]⁺.

### Example 1n: Synthesis of 4-(4-(6,6-difluoro-1,4-diazepan-1-yl)-2-(((S)-1-methylpyrrolidin-2-yl)methoxy)-5,6,7,8-tetrahydroquinazolin-7-yl)benzo[d]thiazol-2-amine (Compound 432)

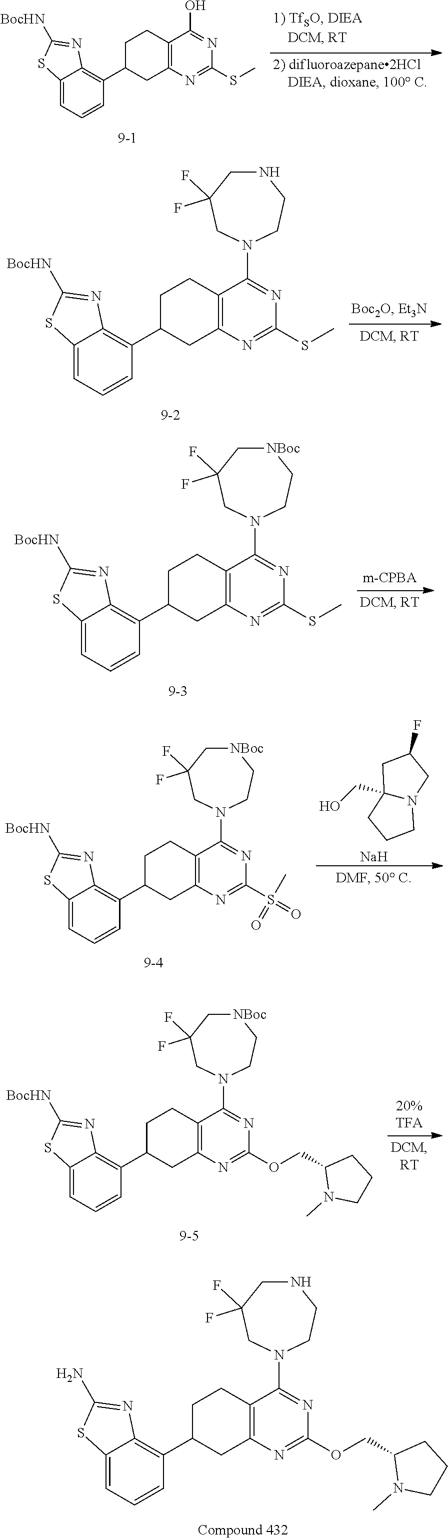

To a stirred solution of tert-butyl (4-(4-hydroxy-2-(methylthio)-5,6,7,8-tetrahydroquinazolin-7-yl)benzo[d]thiazol-2-yl)carbamate (9-1) (100 mg, 0.22 mmol) in DCM (5 mL) was added DIEA (153 μL, 0.88 mmol) followed by a 1M solution of trifluoromethanesulfonic anhydride in DCM (440 μL, 0.44 mmol). The reaction mixture was stirred at room temperature for 30 min then concentrated. The residue was dissolved in dioxane (5 mL), DIEA (230 μL, 1.32 mmol) was added followed by 6,6-difluoro-1,4-diazepane dihydrochloride (200 mg, 0.96 mmol). The whole reaction mixture was heated at 100° C. for 3 h, concentrated. The residue was purified by silica gel column chromatography eluting with 0-20% methanol/ethyl acetate to afford compound 9-2 (96 mg) as a mixture containing small amount of starting material. ESI-MS m/z: 563.2 [M+H]⁺.

The reaction mixture of compound 9-2 (96 mg, 0.17 mmol), Et₃N (60 μL, 0.44 mmol) and Boc₂O (78 μL, 0.34 mmol) in DCM (4 mL) was stirred at room temperature overnight. Concentrated, the residue was purified by silica gel column chromatography eluting with 0-50% ethyl acetate/hexane to afford compound 9-3 (87 mg). ESI-MS m/z: 663.2 [M+H]⁺.

To a stirred solution of compound 9-3 (87 mg, 0.13 mmol) in DCM (4 mL) was added meta-chloroperoxybenzoic acid (77%) (73 mg, 0.33 mmol) at room temperature. The reaction mixture was stirred for 3 h, diluted with saturated aqueous sodium bicarbonate solution, extracted with DCM. The combined organics were washed with water and concentrated. The residue was purified by silica gel column chromatography eluting with 0-50% ethyl acetate/hexane to afford compound 9-4 (50 mg). ESI-MS m/z: 695.2 [M+H]⁺.

To a stirred solution of N-methyl-L-prolinol (17 mg, 0.14 mmol) in DMF (1 mL) was added sodium hydride (60% suspension in mineral oil) (6 mg, 0.15 mmol) at room temperature under nitrogen. After 15 min, compound 9-4 (25 mg, 0.036 mmol) in DMF (1 mL) was added. The reaction mixture was stirred at 50° ° C. for 3 h, poured into saturated aqueous NH₄Cl solution, extracted with ethyl acetate. The combined organics were washed with water, brine and concentrated. The residue was purified by silica gel column chromatography eluting with 0-100% ethyl acetate/hexane with 0.5% Et3N to afford compound 9-5 (12 mg). ESI-MS m/z: 730.3 [M+H]⁺.

The reaction mixture of compound 9-5 (12 mg, 0.016 mmol) in 20% TFA in DCM (2 mL) was stirred at room temperature for 4 h, concentrated. The residue was purified by HPLC (5-60% CH₃CN/H2O with 0.1% formic acid), the combined fractions were lyophilized to afford compound 432 as formic acid salt (6 mg). ESI-MS m/z: 530.2 [M+H]⁺; 1HNMR (400 MHZ, d6-DMSO): 7.54 (d, J=7.8 Hz, 1H), 7.47 (s, 2H), 7.09 (d, J=7.1 Hz, 1H), 7.00 (t, J=7.6 Hz, 1H), 4.65-4.50 (m, 2H), 4.47-4.37 (m, 1H), 4.05-3.72 (m, 3H), 3.50-3.20 (m, 5H, buried in H₂O peak) 3.68-3.54 (m, 2H), 3.18-3.00 (m, 3H), 2.92 (s, 3H), 2.92-2.84 (m, 1H), 2.80-2.70 (m, 1H), 2.65-2.58 (m, 1H), 2.28-2.15 (m, 1H), 2.10-1.98 (m, 2H), 1.98-1.86 (m, 3H).

Compounds having an 8-membered heterocyclic ring in place of a corresponding 7-membered ring (e.g., at Ring A), can be made according to the exemplary synthesis methods of example 1A, 1B, 1h, 1l, 1m and 1n.

### Example 1o: Synthesis of 7-(2-amino-7-fluorobenzo[d]thiazol-4-yl)-4-((1R,5S)-3,8-diazabicyclo[3.2.1]octan-3-yl)-6-chloro-2-(2-(pyrrolidin-1-yl)ethyl)phthalazin-1(2H)-one (Compound 404)

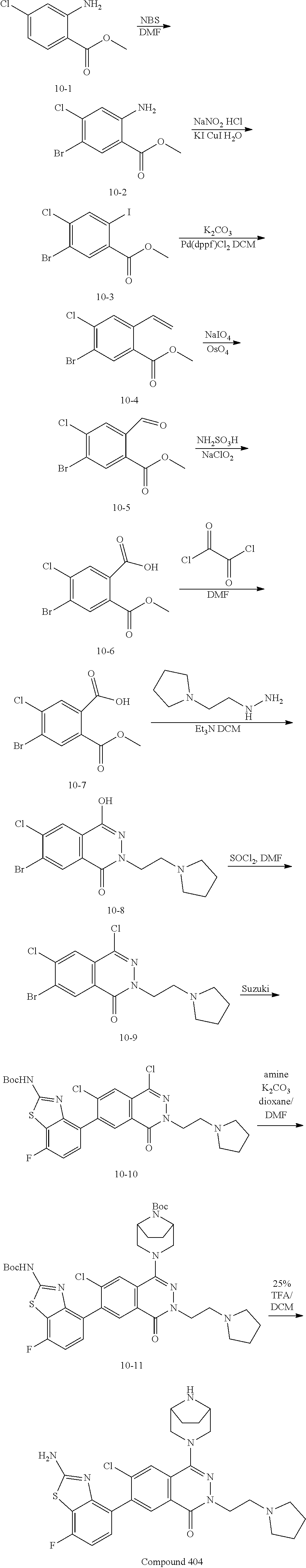

To a solution of (10-1) (10.0 g, 53.88 mmol, 1 eq) in DMF (15 mL) at r, t, NBS (10.1 g, 56.57 mmol, 1.05 eq) was added. The resulting solution was stirred at r, t for 15 h. The mixture was partitioned between dichloromethane and water. The organic layer was concentrated and the residue was purified on a silica gel column eluting with ethyl acetate/petroleum (1:3) to afford compound 10-2 (15.0 g). ESI-MS m/z: 265[M+H]⁺.

To a solution of compound 10-2 (15 g, 56.8 mmol) in HCl/H₂O (150 mL/150 mL), NaNO₂ (5.1 g, 73.8 mmol), KI (42.6 g, 284 mmol) and CuI (21.6 g, 113.6 mmol) were added. The resulting mixture was stirred for 15 h. The mixture was partitioned between ethyl acetate and water. The organic layer was concentrated. The residue was purified on a silica gel column to afford compound 10-3 (6.0 g). ESI-MS m/z: 376 [M+H]⁺.

The mixture of compound 10-3 (3.0 g, 8.0 mmol), potassium vinyltrifluoroborate (1.07 g, 8.0 mmol), K₂CO₃ (3.3 g, 24 mmol), Pd(dppf)Cl₂DCM (654 mg, 0.8 mmol) in dioxane/water (3:1) was stirred at 100° C. under argon overnight. The resulting mixture was diluted with water (20 mL) and extracted with EtOAc (50 mL×2). The combined organic layer was washed with water and brine, dried over anhydrous Na₂SO₄, filtered and concentrated in vacuo. The residue was purified by a silica gel column to afford compound 10-4 (2.1 g), ESI-MS m/z: 276 [M+H]⁺.

To a stirred solution of compound 10-4 (2.1 g) in MeOH/water/THF (4:2:1, 20 mL), was added NaIO₄ (4.9 g, 22.9 mmol), 0.0 and the resulting mixture was stirred at RT for 15 h. The resulting mixture was diluted with water (20 mL) and extracted with EtOAc (50 mL×2). The combined organic layer was washed with water and brine, dried over anhydrous Na₂SO₄, filtered and concentrated in vacuo. The residue was purified by a silica gel column to afford compound 10-5 (2.0 g). ESI-MS m/z: 278[M+H]⁺.

The mixture of compound 10-5 (2.0 g, 7.2 mmol), NH₂SO₃H (1.4 g, 14.4 mmol), NaClO₂ (1.3 g, 14.4 mmol), in dioxane/water (3:1, 40 mL) was stirred overnight. The resulting mixture was diluted with water (20 mL) and extracted with EtOAc (50 mL×2). The combined organic layer was washed with water and brine, dried over anhydrous Na₂SO₄, filtered and concentrated in vacuo. The residue was purified by a silica gel column to afford compound 10-6 (1.2 g). ESI-MS m/z: 294[M+H]⁺.

To a mixture of compound 10-6 (500 mg, 1.7 mmol) in DCM (5 mL), were added oxalyl dichloride (432 mg, 340 mmol) and DMF (2 drops), and the resulting mixture was stirred for 2 h. The mixture was concentrated in vacuo to obtain compound 10-7 (500 mg). ESI-MS m/z: 312 [M+H]⁺.

The mixture of compound 10-7 (500 mg, 1.6 mmol) in DCM (10 mL), 2-(2-(pyrrolidin-1-yl)ethyl)hydrazine (417 mg, 3.2 mmol) and Et₃N (485 mg, 4.8 mmol) were added and the resulting mixture was stirred for 2 h. The mixture was diluted with water (20 mL) and extracted with EtOAc (50 mL×2). The combined organic layer was washed with water and brine, dried over anhydrous Na₂SO₄, filtered and concentrated in vacuo. The residue was purified by a silica gel column to afford compound 10-8 (330 mg). ESI-MS m/z: 374 [M+H]⁺.

To a reaction mixture of compound 10-8 (120 mg, 0.32 mmol) in thionyl chloride (6 mL) was added a few drops of DMF at room temperature. The whole reaction mixture was stirred at 75° C. for one day. Concentrated, the residue was purified by silica gel column chromatography with 0-80% 10% methanol in ethyl acetate with 0.5% Et₃N to afford compound 10-9 (90 mg). ESI-MS m/z: 390.0, 391.9 [M+H]⁺.

The reaction mixture of compound 10-9 (90 mg, 0.23 mmol), (2-((tert-butoxycarbonyl)amino)-7-fluorobenzo[d]thiazol-4-yl)boronic acid (108 mg, 0.34 mmol), K₃PO₄ (88 mg, 0.41 mmol) and dichloro[1,1′-bis(di-tert-butylphosphino)ferrocene]palladium (II) (28 mg, 0.05 mmol) in a mixed solvent of dioxane-water (2 mL, 3:1) was heated at 90° C. for 2 h under nitrogen. Filtered through Celite, the filtrate was concentrated, the residue was purified by silica gel column chromatography with 0-100% 10% methanol in ethyl acetate with 0.5% Et₃N to afford compound 10-10 (95 mg). ESI-MS m/z: 578.1 [M+H]⁺.

To a stirred solution of compound 10-10 (20 mg, 0.035 mmol) in a mixed solvent dioxane-DMF (2 ml, 1:1) was added tert-butyl (1R,5S)-3,8-diazabicyclo[3.2.1]octane-8-carboxylate (110 mg, 0.52 mmol) followed by K₂CO₃ (50 mg, 0.35 mmol). The reaction mixture was heated at 100° ° C. for 2 days, still some starting material left, 110 mg more of amine and 50 mg more of K₂CO₃ were added, stirred for another day. The reaction was complete. Filtered, washed with ethyl acetate. The filtrate was concentrated, the residue was purified by silica gel column chromatography with 0-100% 10% methanol in ethyl acetate with 0.5% Et₃N to afford compound 10-11 (14 mg). ESI-MS m/z: 754.5 [M+H]⁺.

The reaction mixture of compound 10-11 (14 mg, 0.019 mmol) in 25% TFA in DCM (2 mL) was stirred at room temperature for 4 h, concentrated. The residue was purified by HPLC (5-60% CH₃CN/H₂O with 0.1% formic acid). The combined fractions were lyophilized to afford compound 404 as formic acid salt (8 mg). ESI-MS m/z: 554.3 [M+H]⁺.

### Example 1p: Synthesis of 4-(6-chloro-8-fluoro-2-(((2R,7aS)-2-fluorotetrahydro-1H-pyrrolizin-7a(5H)-yl)methoxy)-4-((1R,5S)-8-methyl-3,8-diazabicyclo[3.2.1]octan-3-yl)quinazolin-7-yl)-7-fluorobenzo[d]thiazol-2-amine (Compound 470)

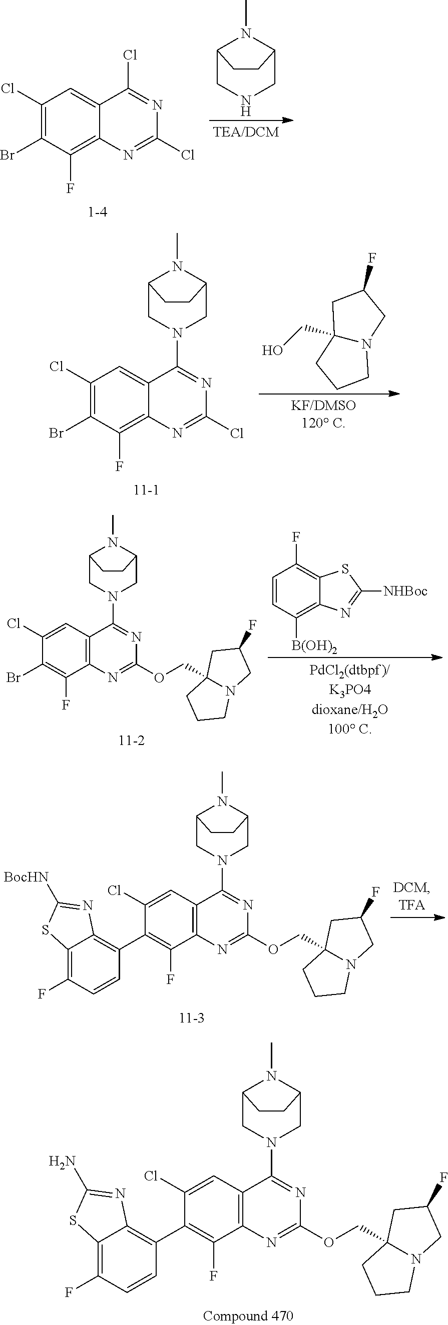

To a solution of compound 1-4 (220 mg, 0.67 mmol) in DCM (20 mL), TEA (339 mg, 3.36 mmol) and (1R,5S)-8-methyl-3,8-diazabicyclo[3.2.1]octane (101 mg, 0.81 mmol) were added and the resulting mixture was stirred at RT overnight. The mixture was partitioned between water and DCM. The organic layer was washed with brine, dried over Na₂SO₄, filtered and concentrated in vacuo. The residue was purified by flash column chromatography on silica gel to afford compound 11-1 (220 mg). ESI-MS m/z: 417.98 [M+H]⁺.

To a stirred solution of compound 11-1 (200 mg, 0.48 mmol) in DMSO (4 mL) under nitrogen, KF (222 mg, 3.83 mmol) and ((2R,7aS)-2-fluorotetrahydro-1H-pyrrolizin-7a(5H)-yl)methanol (114 mg, 0.72 mmol) were added and the resulting mixture was stirred at 120° C. for 3 h. The mixture was partitioned between water and ethyl acetate. The organic layer was washed with brine, dried over Na₂SO₄, filtered and concentrated in vacuo. The residue was purified by flash column chromatography on silica gel to afford product 11-2 (100 mg). ESI-MS m/z: 541.11 [M+H]⁺.

To a stirred solution of compound 11-2 (100 mg, 0.18 mmol) in 1,4-dioxane (8 mL) and H₂O (2 mL), (2-((tert-butoxycarbonyl)amino)-7-fluorobenzo[d]thiazol-4-yl)boronic acid (86 mg, 0.28 mmol), PdCl₂(dtbpf) (18 mg, 0.028 mmol) and K₃PO₄ (137 mg, 0.65 mmol) were added and the resulting mixture was stirred at 100° C. under nitrogen for 3 h. The mixture was partitioned between water and ethyl acetate. The organic layer was washed with brine, dried over Na₂SO₄, filtered and concentrated in vacuo. The residue was purified by flash column chromatography on silica gel to afford product 11-3 (30 mg). ESI-MS m/z: 729.20 [M+H]⁺.

To a solution of compound 11-3 (30 mg) in DCM (6 mL) at RT, TFA (2 mL) was added and the resulting mixture was stirred at RT for 30 min. The mixture was concentrated in vacuo to remove the solvent. The residue was purified by flash column chromatography on silica gel to afford compound 470 (5.0 mg). ESI-MS m/z: 630.5 [M+H]⁺; 1HNMR (400 MHZ, CD₃OD): δ 8.38 (s, 1H), 7.80 (s, 1H), 7.14-7.10 (m, 1H), 6.92-6.87 (m, 1H), 5.41-5.28 (m, 1H), 4.49-4.35 (m, 5H), 3.66-3.34 (m, 7H), 2.35-1.19 (m, 13H).

### Example 1q: Synthesis of 4-(5-((1R,5S)-3,8-diazabicyclo[3.2.1]octan-3-yl)-7-(((2R,7aS)-2-fluorotetrahydro-1H-pyrrolizin-7a(5H)-yl)methoxy)pyrimido[4,5-d]pyrimidin-2-yl)naphthalen-2-ol (Compound 418)

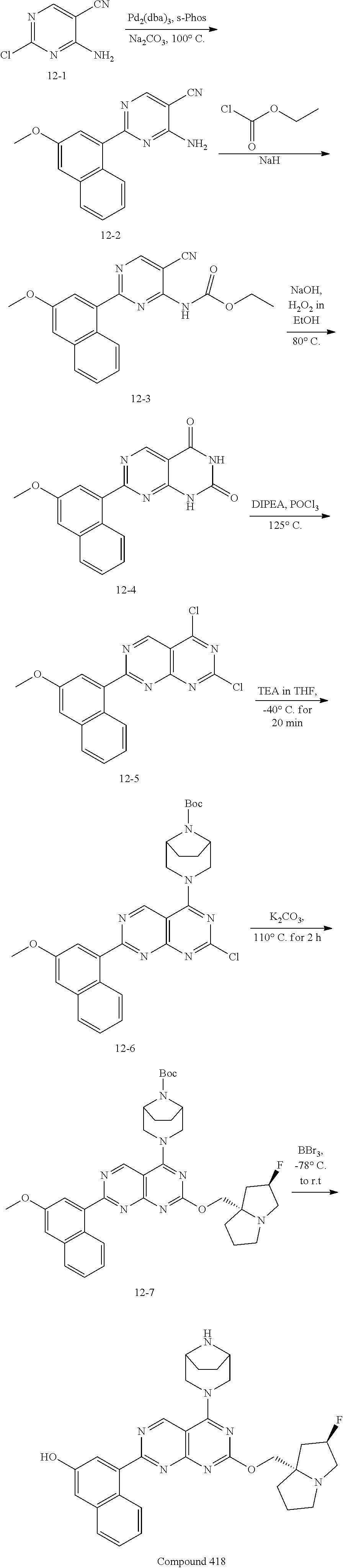

The mixture of compound 12-1 (2 g, 12.94 mmol), (3-methoxynaphthalen-1-yl)boronic acid (5.22 g, 25.88 mmol), sodium carbonate (4.80 g, 45.29 mmol), Pd₂(dba); (2.48 g, 2.59 mmol) and S-phos (1.06 g, 2.59 mmol) in toluene and H₂O (20 mL/3 mL) was stirred at 100° ° C. under argon for overnight. The mixture was concentrated in vacuo. The residue was purified by flash column chromatography on silica gel to afford product 12-2 (1.6 g), ESI-MS m/z: 277 [M+H]⁺.

To a solution of compound 12-2 (1.6 g, 5.80 mmol) in THF (30 mL) under nitrogen at 0° C., NaH (60% in paraffin oil, 811 mg, 20.29 mmol) was added in portions and the resulting mixture was stirred at r.t for 2h. Then ethyl carbonochloridate (940 mg, 8.70 mmol) in THF (10 mL) was added dropwise. The mixture was stirred at r.t for overnight. The reaction mixture was quenched with saturated aqueous NH₄Cl, extracted with ethyl acetate, washed with water and brine, dried over Na₂SO₄, filtered and concentrated in vacuo. The residue was purified by flash column chromatography on silica gel to afford product 12-3 (800 mg). ESI-MS m/z: 349 [M+H]⁺.

To a stirred solution of compound 12-3 (8 00 mg, 2.30 mmol) in EtOH (10 mL), NaOH (184 mg, 4.6 mmol) and H₂O₂ (10 ml, 30%) were added and the resulting mixture was stirred at 85° ° C. for 2 h. The reaction mixture was quenched with H₂O (10 ml), concentrated in vacuo, and filtered. The solid was washed with H₂O and dried gel to afford product 12-4 (600 mg). ESI-MS m/z: 321 [M+H]⁺.

To a stirred solution of compound 12-4 (600 mg, 1.87 mmol) in POCl₃ (10 ml) and DIEA (266 mg, 2.06 mmol) were added and the resulting mixture was stirred at 125° C. for overnight. The mixture was concentrated in vacuo. The residue was dissolved in DCM. The residue was purified by flash column chromatography on silica gel to afford product 12-5 (400 mg). ESI-MS m/z: 357 [M+H]⁺.

To a stirred solution of compound 12-5 (400 mg, 1.12 mmol) in THF (10 mL) at −40° C., TEA (340 mg, 3.37 mmol) was added and the (1R,5S)-tert-butyl 3,8-diazabicyclo[3.2.1]octane-8-carboxylate (118 mg, 0.56 mmol) in THF (2 mL) was added dropwise. The resulting mixture was stirred at −40° C. for 20 min. The reaction was quenched with water and extracted with EtOAc. The combined organic layer was washed with brine, dried over Na₂SO₄, filtered and concentrated in vacuo. The residue was purified by flash column chromatography on silica gel to afford product 12-6 (250 mg). ESI-MS m/z: 533 [M+H]⁺.

To a stirred solution of compound 12-6 (250 mg, 0.47 mmol) in DMF (4 mL) under nitrogen, K₂CO₃ (260 mg, 1.88 mmol) and ((2R,7aS)-2-fluorohexahydro-1H-pyrrolizin-7a-yl)methanol (150 mg, 0.94 mmol) were added and the resulting mixture was stirred at 110° C. for 2 h. The mixture was partitioned between water and ethyl acetate. The organic layer was washed with brine, dried over Na₂SO₄, filtered and concentrated in vacuo. The residue was purified by flash column chromatography on silica gel to afford product 12-7 (100 mg). ESI-MS m/z: 656 [M+H]⁺.

To a stirred solution of compound 12-7 (40 mg, 0.06 mmol) in DCM (10 mL) at −78° C., BBr₃ (305 mg, 1.22 mmol) in DCM (2 mL) was added and the resulting mixture was stirred at r.t for overnight. The reaction was re-cooled to −78° C. and quenched with MeOH and 7N NH₃·MeOH added to PH=7. The organic layer was concentrated in vacuo. The residue was purified by flash column chromatography on silica gel to afford compound 418 (5 mg). ESI-MS m/z: 542.5 [M+H]⁺.

### Example 1r: Synthesis of 4-(4-((1R,5S)-3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol (Compound 701)

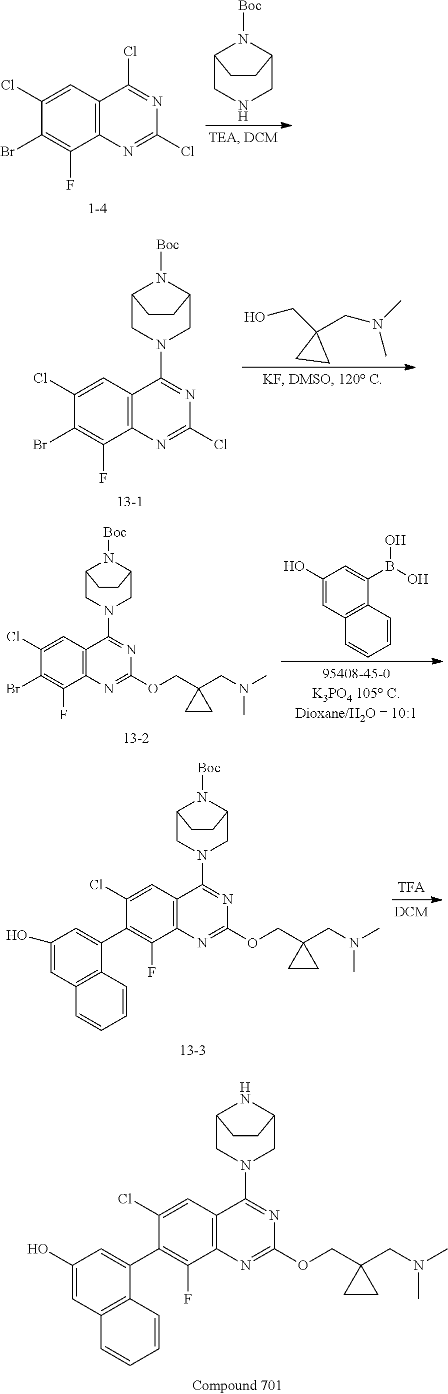

To a solution of compound 1-4 (1.5 g, 4.58 mmol, 1 eq) in dichloromethane (100 mL) at 0° C., were added TEA (1.39 g, 13.74 mmol, 3 eq) and tert-butyl (1R,5S)-3,8-diazabicyclo[3.2.1]octane-8-carboxylate (1.17 g, 5.49 mmol, 1.2 eq). The resulting solution was stirred at 0° C. for 1 h. The mixture was partitioned between dichloromethane and water. The organic layer was concentrated and the residue was purified on a silica gel column eluting with ethyl acetate/petroleum (1:4) to afford product 13-1 (1.5 g). ESI-MS m/z: 507.00 [M+H]⁺.

To a solution of compound 13-1 (210 mg, 0.42 mmol) in DMSO (4 mL) was added KF (194 mg, 3.35 mmol, 8 eq) and (1-((dimethylamino)methyl)cyclopropyl)methanol (108 mg, 0.84 mmol, 2 eq). The resulting mixture was stirred at 120° C. under argon for 3 h. The mixture was partitioned between ethyl acetate and water. The organic layer was concentrated. The residue was purified on a silica gel column to afford compound 13-2 (32 mg). ESI-MS m/z: 598.15 [M+H]⁺.

The mixture of compound 13-2 (32 mg, 0.051 mmol), compound (2-((tert-butoxycarbonyl)amino)-7-fluorobenzo[d]thiazol-4-yl)boronic acid (32 mg, 0.10 mmol), K₃PO₄ (33 mg, 0.15 mmol), PdCl₂(dtbf) (7 mg, 0.01 mmol) in dioxane (10 mL) and water (1 mL) was stirred at 105° C. under argon for 3 h. The resulting mixture was diluted with water (20 mL) and extracted with EtOAc (15 mL×2). The combined organic layer was washed with water and brine, dried over anhydrous Na₂SO₄, filtered and concentrated in vacuo. The residue was purified by prep-TLC to afford product 13-3 (36 mg). ESI-MS m/z: 662.28 [M+H]⁺.

To a stirred solution of compound 13-3 (36 mg, 0.056 mmol) in DCM (6 mL), was a added trifluoroacetic acid (3 mL) and the resulting mixture was stirred at RT for 2 h. The mixture was concentrated in vacuo. The residue was dissolved in DCM. The mixture was concentrated and the residue was purified by prep-TLC plate to afford the compound 701 (3 mg). ESI-MS m/z: 562.5 [M+H]⁺; 1HNMR (400 MHZ, DMSO-d6): δ 8.29 (s, 3H), 7.93 (s, 1H), 7.80 (d, J=8.4 Hz, 1H), 7.46-7.42 (m, 1H), 7.29 (m, 1H), 7.22 (m, 2H), 7.06 (m, 1H), 4.35-4.32 (m, 2H), 4.21 (s, 2H), 3.63-3.57 (m, 4H), 2.24 (s, 2H), 2.17 (s, 6H), 1.69 (s, 4H), 0.63 (s, 2H), 0.40 (s, 2H).

### Example 1s: Synthesis of 2-amino-4-(6-chloro-4-(2-((E)-4-(dimethylamino)but-2-enoyl)-2,6-diazaspiro[3.5]nonan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Compound 522)

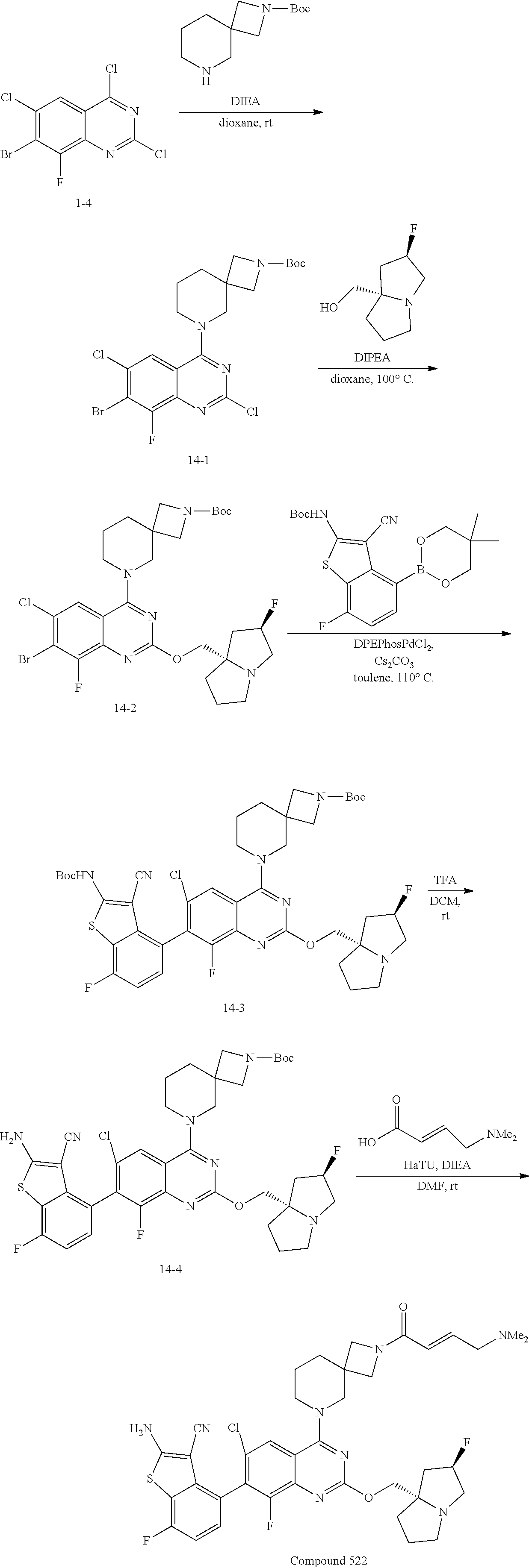

To a solution of compound 1-4 (1.0 g, 3.03 mmol) and tert-butyl 2,6-diazaspiro[3.5]nonane-2-carboxylate (685 mg, 3.03 mmol) in i-PrOH (30 ml) was added DIEA (1.17 g 12.09 mmol). The reaction mixture was stirred at 25° ° C. for 3 h. The mixture was poured into ice water (200 mL). The mixture was filtered and the solid was diluted with MeOH (150 ml). The organic solution was concentrated to dryness under reduced pressure to give compound 14-1 (1.2 g). MS m/z (ESI): 521.1 [M+H]⁺.

To a solution of compound 14-1 (1.0 g, 1.9 mmol) and ((2R,7aS)-2-fluorotetrahydro-1H-pyrrolizin-7a(5H)-yl)methanol (1.53 g, 9.6 mmol) in DMSO (10 ml) was added KF (557 mg, 9.6 mmol). The reaction mixture was stirred at 120° C. for 12 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 14-2 (520 mg). MS m/z (ESI): 644.2 [M+H]⁺.

To a solution of compound 14-2 (500 mg, 0.39 mmol), tert-butyl (3-cyano-4-(5,5-dimethyl-1,3,2-dioxaborinan-2-y1)-7-fluorobenzo[b]thiophen-2-yl)carbamate (195 mg, 0.585 mmol), and Cs₂CO₃ (380 mg, 1.17 mmol) in toluene (10 mL) was added Pd(DPEPhos)Cl₂ (60 mg, 0.078 mmol). The mixture was stirred at 110° C. under nitrogen for 3 hours. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 14-3 (320 mg). MS m/z (ESI): 854.4 [M+H]⁺.

To a solution of compound 14-3 (320 mg, 0.12 mmol) in DCM (5 ml) was added TFA (2 ml). The reaction mixture was stirred at 25° ° C. for 2 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 14-4 (250 mg). MS m/z (ESI): 654.2 [M+H]⁺.

To a solution of compound 14-4 (20 mg, 0.031 mmol) in DMF (5 mL) was added compound 9 (8 mg, 0.0465 mmol), HATU (12 mg, 0.031 mmol) and DIEA (12 mg, 0.093 mmol). The mixture was stirred at 25° C. for 2 h. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by Prep-HPLC (basic) to give compound 522 (2.40 mg). MS m/z (ESI): 756.3 [M+H]⁺. 1H NMR (400 MHZ, DMSO-d6): δ 8.12 (s, 2H), 7.87-7.86 (m, 1H), 7.26-7.23 (m, 1H), 7.19-7.14 (m, 1H), 6.61-6.59 (m, 1H), 6.42-6.36 (m, 1H), 5.30 (d, J=52.4 Hz, 1H), 4.51 (s, 2H), 4.03-3.94 (m, 4H), 3.90-3.80 (m, 4H), 3.72-3.67 (m, 6H), 2.71-2.67 (m, 6H), 2.42-2.28 (m, 3H), 2.13-1.99 (m, 3H), 1.93 (m, 2H), 1.78 (m, 2H).

The compounds in Table 1 were synthesized using similar procedures as shown in the preceding examples.

TABLE 1

| | | | ESI-MS |
|---|---|---|---|
| | | | m/z |
| Compound | Structure | Name | [M + H]⁺ |
| 102 | 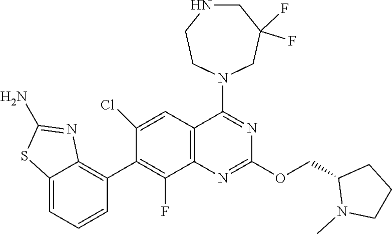 [SMILES: CN1CCC[C@H]1COc1nc(N2CCNCC(F)(F)C2)c2cc(Cl)c(-c3cccc4sc(N)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]thiazol-2-amine | 542.2 |
| 103 | 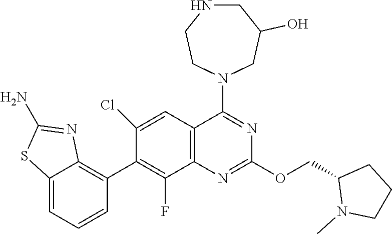 [SMILES: CN1CCC[C@H]1COc1nc(N2CCNCC(O)C2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)-1,4- diazepan-6-ol | 558.2 |
| 104 | 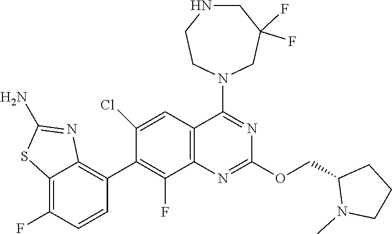 [SMILES: CN1CCC[C@H]1COc1nc(N2CCNCC(F)(F)C2)c2cc(Cl)c(-c3ccc(F)c4sc(N)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-7-fluorobenzo[d]thiazol- 2-amine | 596.1 |
| 105 | 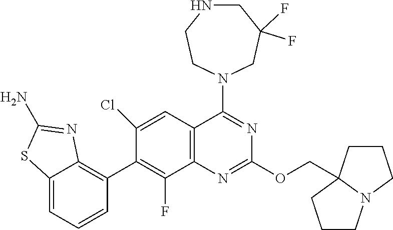 [SMILES: Nc1nc2c(-c3c(Cl)cc4c(N5CCNCC(F)(F)C5)nc(OCC56CCCN5CCC6)nc4c3F)cccc2s1] | 4-(6-chloro-4-(6,6-difluoro- 1,4-diazepan-1-yl)-8-fluoro- 2-((tetrahydro-1H-pyrrol- izin-7a(5H)-yl)methoxy)- quinazolin-7-yl)benzo[d]- thiazol-2-amine | 604.3 |
| 106 | 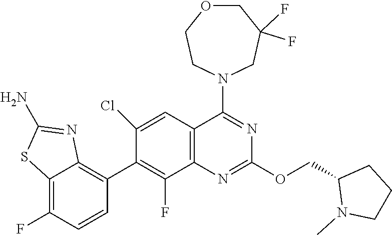 [SMILES: CN1CCC[C@H]1COc1nc(N2CCOCC(F)(F)C2)c2cc(Cl)c(-c3ccc(F)c4sc(N)nc34)c(F)c2n1] | 4-(6-chloro-4-(6,6-difluoro- 1,4-oxazepan-4-yl)-8-fluoro- 2-(((S)-1-methylpyrrolidin- 2-yl)methoxy)quinazolin-7- yl)-7-fluorobenzo[d]thiazol- 2-amine | 597.2 |
| 107 | 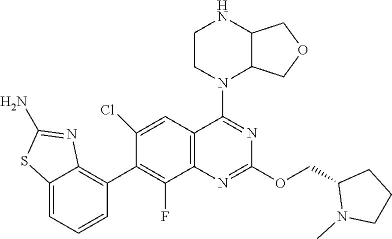 [SMILES: CN1CCC[C@H]1COc1nc(N2CCNC3COCC32)c2cc(Cl)c(-c3cccc4sc(N)nc34)c(F)c2n1] | 4-(6-chloro-8-fluoro-4- (hexahydrofuro[3,4-b]pyr- azin-1(2H)-yl)-2-(((S)-1- methylpyrrolidin-2-yl)meth- oxy)quinazolin-7-yl)benzo- [d]thiazol-2-amine | 570.7 |
| 108 | 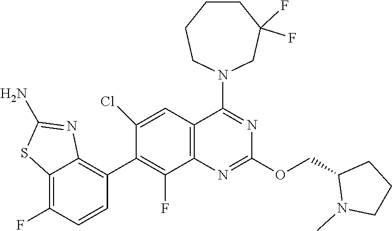 [SMILES: CN1CCC[C@H]1COc1nc(N2CCCCC(F)(F)C2)c2cc(Cl)c(-c3ccc(F)c4sc(N)nc34)c(F)c2n1] | 4-(6-chloro-4-(3,3-difluoro- azepan-1-yl)-8-fluoro-2- (((S)-1-methylpyrrolidin-2- yl)methoxy)quinazolin-7- yl)-7-fluorobenzo[d]thiazol- 2-amine | 595.2 |
| 109 | 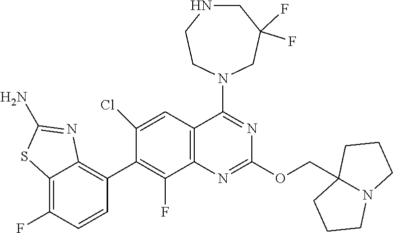 [SMILES: Nc1nc2c(-c3c(Cl)cc4c(N5CCNCC(F)(F)C5)nc(OCC56CCCN5CCC6)nc4c3F)ccc(F)c2s1] | 4-(6-chloro-4-(6,6-difluoro- 1,4-diazepan-1-yl)-8-fluoro- 2-((tetrahydro-1H-pyrrol- izin-7a(5H)-yl)methoxy)- quinazolin-7-yl)-7-fluoro- benzo[d]thiazol-2-amine | 622.1 |
| 110 | 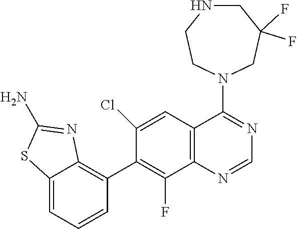 [SMILES: Nc1nc2c(-c3c(Cl)cc4c(N5CCNCC(F)(F)C5)ncnc4c3F)cccc2s1] | 4-(6-chloro-4-(6,6-difluoro- 1,4-diazepan-1-yl)-8-fluoro- quinazolin-7-yl)benzo[d]- thiazol-2-amine | 465.4 |
| 111 | 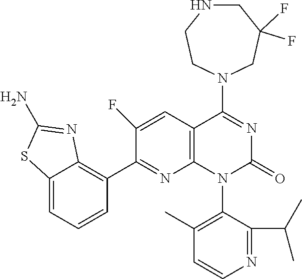 [SMILES: Cc1ccnc(C(C)C)c1-n1c(=O)nc(N2CCNCC(F)(F)C2)c2cc(F)c(-c3cccc4sc(N)nc34)nc21] | 7-(2-aminobenzo[d]thiazol- 4-yl)-4-(6,6-difluoro-1,4- diazepan-1-yl)-6-fluoro-1- (2-isopropyl-4-methylpyr- idin-3-yl)pyrido[2,3-d]pyr- imidin-2(1H)-one | 581.3 |
| 112 | 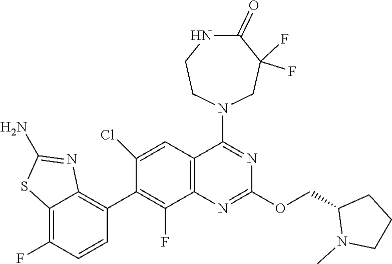 [SMILES: CN1CCC[C@H]1COc1nc(N2CCNC(=O)C(F)(F)C2)c2cc(Cl)c(-c3ccc(F)c4sc(N)nc34)c(F)c2n1] | 1-(7-(2-amino-7-fluoro- benzo[d]thiazol-4-yl)-6- chloro-8-fluoro-2-(((S)-1- methylpyrrolidin-2-yl)meth- oxy)quinazolin-4-yl)-6,6- difluoro-1,4-diazepan-5- one | 610.1 |
| 113 | 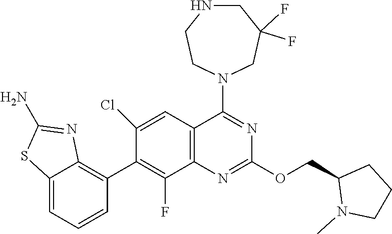 [SMILES: CN1CCC[C@@H]1COc1nc(N2CCNCC(F)(F)C2)c2cc(Cl)c(-c3cccc4sc(N)nc34)c(F)c2n1] | 4-(6-chloro-4-(6,6-difluoro- 1,4-diazepan-1-yl)-8-fluoro- 2-(((R)-1-methylpyrrolidin- 2-yl)methoxy)quinazolin-7- yl)benzo[d]thiazol-2-amine | 576.4 [M − H]⁻ |
| 114 | 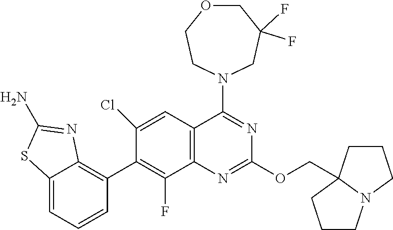 [SMILES: Nc1nc2c(-c3c(Cl)cc4c(N5CCOCC(F)(F)C5)nc(OCC56CCCN5CCC6)nc4c3F)cccc2s1] | 4-(6-chloro-4-(6,6-difluoro- 1,4-oxazepan-4-yl)-8-fluoro- 2-((tetrahydro-1H-pyrrolizin- 7a(5H)-yl)methoxy)quin- azolin-7-yl)benzo[d]thiazol- 2-amine | 605.8 |
| 115 | 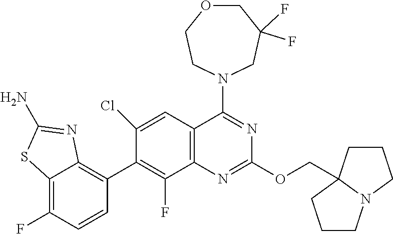 [SMILES: Nc1nc2c(-c3c(Cl)cc4c(N5CCOCC(F)(F)C5)nc(OCC56CCCN5CCC6)nc4c3F)ccc(F)c2s1] | 4-(6-chloro-4-(6,6-difluoro- 1,4-oxazepan-4-yl)-8-fluoro- 2-((tetrahydro-1H-pyrrol- izin-7a(5H)-yl)methoxy)- quinazolin-7-yl)-7-fluoro- benzo[d]thiazol-2-amine | 623.7 |
| 116 | 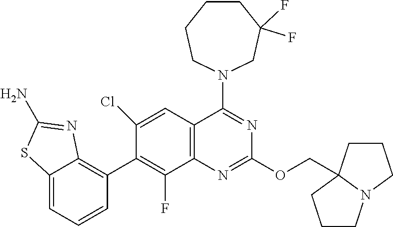 [SMILES: Nc1nc2c(-c3c(Cl)cc4c(N5CCCCC(F)(F)C5)nc(OCC56CCCN5CCC6)nc4c3F)cccc2s1] | 4-(6-chloro-4-(3,3-difluoro- azepan-1-yl)-8-fluoro-2- ((tetrahydro-1H-pyrrolizin- 7a(5H)-yl)methoxy)quin- azolin-7-yl)benzo[d]thiazol- 2-amine | 603.2 |
| 117 | 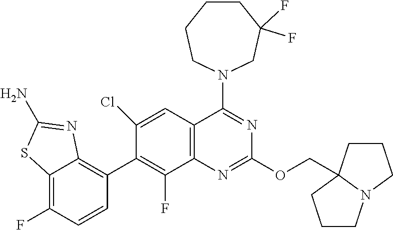 [SMILES: Nc1nc2c(-c3c(Cl)cc4c(N5CCCCC(F)(F)C5)nc(OCC56CCCN5CCC6)nc4c3F)ccc(F)c2s1] | 4-(6-chloro-4-(3,3-difluoro- azepan-1-yl)-8-fluoro-2- ((tetrahydro-1H-pyrrolizin- 7a(5H)-yl)methoxy)quin- azolin-7-yl)-7-fluorobenzo- [d]thiazol-2-amine | 621.2 |
| 118 | 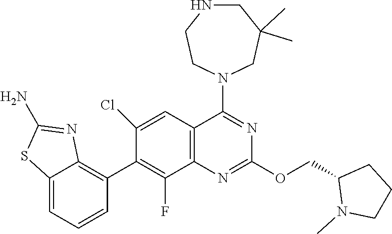 [SMILES: CN1CCC[C@H]1COc1nc(N2CCNCC(C)(C)C2)c2cc(Cl)c(-c3cccc4sc(N)nc34)c(F)c2n1] | 4-(6-chloro-4-(6,6-dimethyl- 1,4-diazepan-1-yl)-8-fluoro- 2-(((S)-1-methylpyrrolidin- 2-yl)methoxy)quinazolin-7- yl)benzo[d]thiazol-2-amine | 570.5 |
| 119 | 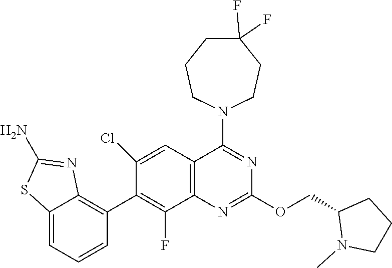 [SMILES: CN1CCC[C@H]1COc1nc(N2CCCC(F)(F)CC2)c2cc(Cl)c(-c3cccc4sc(N)nc34)c(F)c2n1] | 4-(6-chloro-4-(4,4-difluoro- azepan-1-yl)-8-fluoro-2- (((S)-1-methylpyrrolidin-2- yl)methoxy)quinazolin-7-yl)- benzo[d]thiazol-2-amine | 577.5 |
| 120 | 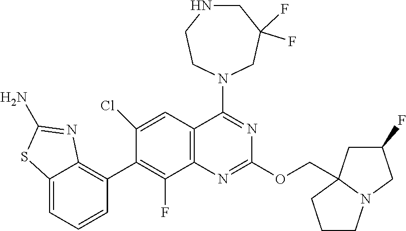 [SMILES: Nc1nc2c(-c3c(Cl)cc4c(N5CCNCC(F)(F)C5)nc(OCC56CCCN5C[C@H](F)C6)nc4c3F)cccc2s1] | 4-(6-chloro-4-(6,6-difluoro- 1,4-diazepan-1-yl)-8-fluoro- 2-(((2R)-2-fluorotetrahydro- 1H-pyrrolizin-7a(5H)-yl)- methoxy)quinazolin-7-yl)- benzo[d]thiazol-2-amine | 620.3 [M − H]⁻ |
| 121 | 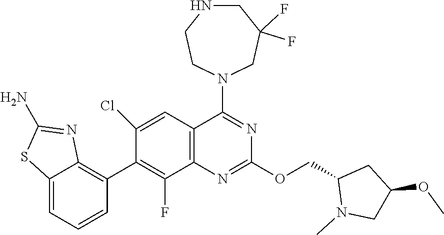 [SMILES: CO[C@@H]1C[C@@H](COc2nc(N3CCNCC(F)(F)C3)c3cc(Cl)c(-c4cccc5sc(N)nc45)c(F)c3n2)N(C)C1] | 4-(6-chloro-4-(6,6-difluoro- 1,4-diazepan-1-yl)-8-fluoro- 2-(((2S,4R)-4-methoxy-1- methylpyrrolidin-2-yl)- methoxy)quinazolin-7-yl)- benzo[d]thiazol-2-amine | 606.5 [M − H]⁻ |
| 122 | 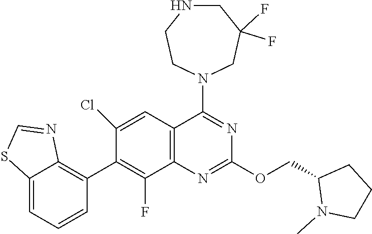 [SMILES: CN1CCC[C@H]1COc1nc(N2CCNCC(F)(F)C2)c2cc(Cl)c(-c3cccc4scnc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]thiazole | 563.3 |
| 123 | 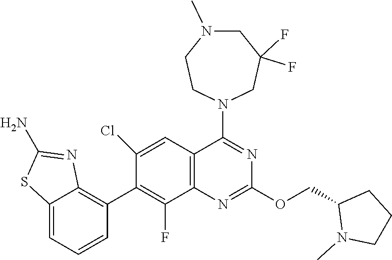 [SMILES: CN1CCN(c2nc(OC[C@@H]3CCCN3C)nc3c(F)c(-c4cccc5sc(N)nc45)c(Cl)cc23)CC(F)(F)C1] | 4-(6-chloro-4-(6,6-difluoro- 4-methyl-1,4-diazepan-1-yl)- 8-fluoro-2-(((S)-1-methyl- pyrrolidin-2-yl)methoxy)- quinazolin-7-yl)benzo[d]- thiazol-2-amine | 592.1 |
| 124 | 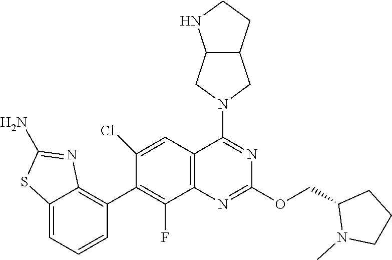 [SMILES: CN1CCC[C@H]1COc1nc(N2CC3CCNC3C2)c2cc(Cl)c(-c3cccc4sc(N)nc34)c(F)c2n1] | 4-(6-chloro-8-fluoro-4- (hexahydropyrrolo[3,4-b]- pyrrol-5(1H)-yl)-2-(((S)-1- methylpyrrolidin-2-yl)meth- oxy)quinazolin-7-yl)benzo- [d]thiazol-2-amine | 554.2 |
| 125 | 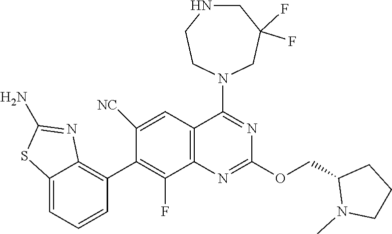 [SMILES: CN1CCC[C@H]1COc1nc(N2CCNCC(F)(F)C2)c2cc(C#N)c(-c3cccc4sc(N)nc34)c(F)c2n1] | 7-(2-aminobenzo[d]thiazol- 4-yl)-4-(6,6-difluoro-1,4- diazepan-1-yl)-8-fluoro-2- (((S)-1-methylpyrrolidin-2- yl)methoxy)quinazoline-6- carbonitrile | 569.4 |
| 126 | 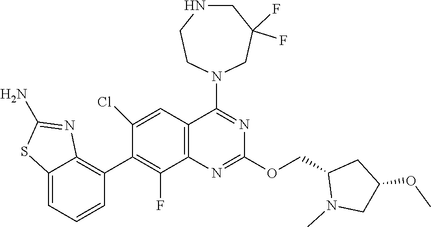 [SMILES: CO[C@H]1C[C@@H](COc2nc(N3CCNCC(F)(F)C3)c3cc(Cl)c(-c4cccc5sc(N)nc45)c(F)c3n2)N(C)C1] | 4-(6-chloro-4-(6,6-difluoro- 1,4-diazepan-1-yl)-8-fluoro- 2-(((2S,4S)-4-methoxy-1- methylpyrrolidin-2-yl)- methoxy)quinazolin-7-yl)- benzo[d]thiazol-2-amine | 606.3 [M − H]⁻ |
| 127 | 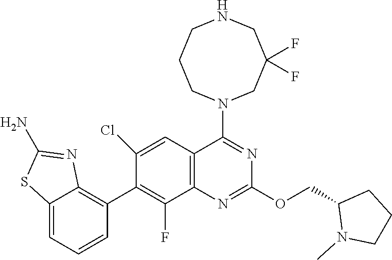 [SMILES: CN1CCC[C@H]1COc1nc(N2CCCNCC(F)(F)C2)c2cc(Cl)c(-c3cccc4sc(N)nc34)c(F)c2n1] | 4-(6-chloro-4-(3,3-difluoro- 1,5-diazocan-1-yl)-8-fluoro- 2-(((S)-1-methylpyrrolidin- 2-yl)methoxy)quinazolin-7- yl)benzo[d]thiazol-2-amine | 592.2 |
| 128 | 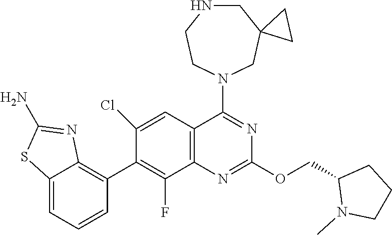 [SMILES: CN1CCC[C@H]1COc1nc(N2CCNCC3(CC3)C2)c2cc(Cl)c(-c3cccc4sc(N)nc34)c(F)c2n1] | 4-(6-chloro-8-fluoro-2-(((S)- 1-methylpyrrolidin-2-yl)- methoxy)-4-(5,8-diazaspiro- [2.6]nonan-5-yl)quinazolin- 7-yl)benzo[d]thiazol-2- amine | 568.2 |
| 129 | 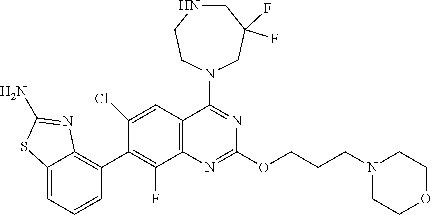 [SMILES: Nc1nc2c(-c3c(Cl)cc4c(N5CCNCC(F)(F)C5)nc(OCCCN5CCOCC5)nc4c3F)cccc2s1] | 4-(6-chloro-4-(6,6-difluoro- 1,4-diazepan-1-yl)-8-fluoro- 2-(3-morpholinopropoxy)- quinazolin-7-yl)benzo[d]- thiazol-2-amine | 608.4 |
| 130 | 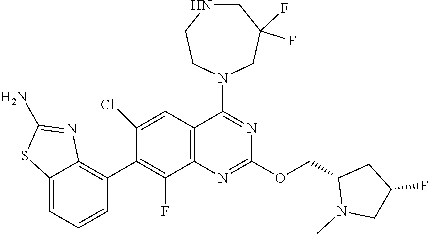 [SMILES: CN1C[C@@H](F)C[C@H]1COc1nc(N2CCNCC(F)(F)C2)c2cc(Cl)c(-c3cccc4sc(N)nc34)c(F)c2n1] | 4-(6-chloro-4-(6,6-difluoro- 1,4-diazepan-1-yl)-8-fluoro- 2-(((2S,4S)-4-fluoro-1- methylpyrrolidin-2-yl)meth- oxy)quinazolin-7-yl)benzo- [d]thiazol-2-amine | 596.4 |
| 131 | 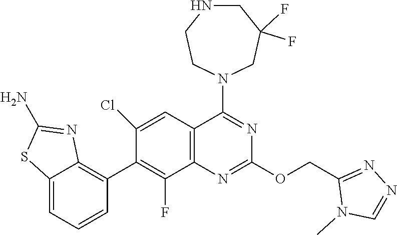 [SMILES: Cn1cnnc1COc1nc(N2CCNCC(F)(F)C2)c2cc(Cl)c(-c3cccc4sc(N)nc34)c(F)c2n1] | 4-(6-chloro-4-(6,6-difluoro- 1,4-diazepan-1-yl)-8-fluoro- 2-((4-methyl-4H-1,2,4- triazol-3-yl)methoxy)quin- azolin-7-yl)benzo[d]thiazol- 2-amine | 576.4 |
| 132 | 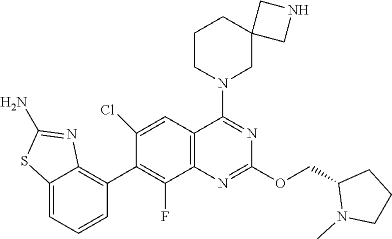 [SMILES: CN1CCC[C@H]1COc1nc(N2CCCC3(CNC3)C2)c2cc(Cl)c(-c3cccc4sc(N)nc34)c(F)c2n1] | 4-(6-chloro-8-fluoro-2-(((S)- 1-methylpyrrolidin-2-yl)- methoxy)-4-(2,6-diazaspiro- [3.5]nonan-6-yl)quinazolin- 7-yl)benzo[d]thiazol-2-amine | 568.2 |
| 133 | 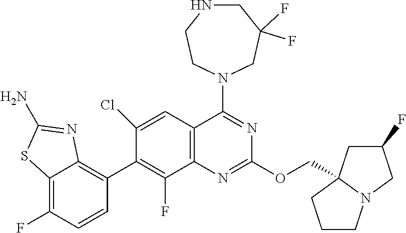 [SMILES: Nc1nc2c(-c3c(Cl)cc4c(N5CCNCC(F)(F)C5)nc(OC[C@@]56CCCN5C[C@H](F)C6)nc4c3F)ccc(F)c2s1] | 4-(6-chloro-4-(6,6-difluoro- 1,4-diazepan-1-yl)-8-fluoro- 2-(((2R,7aS)-2-fluorotetra- hydro-1H-pyrrolizin-7a(5H)- yl)methoxy)quinazolin-7-yl)- 7-fluorobenzo[d]thiazol-2- amine | 640.1 |
| 134 | 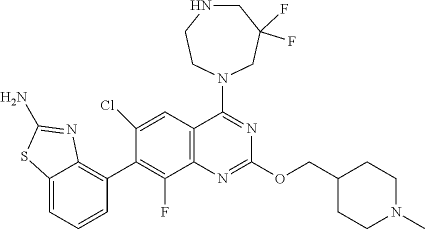 [SMILES: CN1CCC(COc2nc(N3CCNCC(F)(F)C3)c3cc(Cl)c(-c4cccc5sc(N)nc45)c(F)c3n2)CC1] | 4-(6-chloro-4-(6,6-difluoro- 1,4-diazepan-1-yl)-8-fluoro- 2-((1-methylpiperidin-4-yl)- methoxy)quinazolin-7-yl)- benzo[d]thiazol-2-amine | 592.3 |
| 135 | 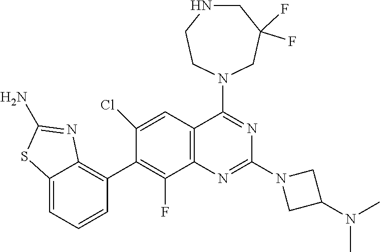 [SMILES: CN(C)C1CN(c2nc(N3CCNCC(F)(F)C3)c3cc(Cl)c(-c4cccc5sc(N)nc45)c(F)c3n2)C1] | 4-(6-chloro-4-(6,6-difluoro- 1,4-diazepan-1-yl)-2-(3- (dimethylamino)azetidin-1- yl)-8-fluoroquinazolin-7-yl)- benzo[d]thiazol-2-amine | 563.4 |
| 136 | 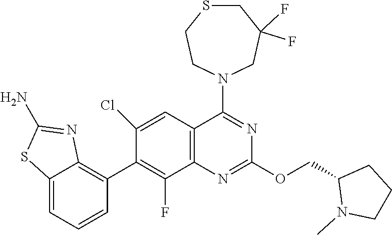 [SMILES: CN1CCC[C@H]1COc1nc(N2CCSCC(F)(F)C2)c2cc(Cl)c(-c3cccc4sc(N)nc34)c(F)c2n1] | 4-(6-chloro-4-(6,6-difluoro- 1,4-thiazepan-4-yl)-8-fluoro- 2-(((S)-1-methylpyrrolidin- 2-yl)methoxy)quinazolin-7- yl)benzo[d]thiazol-2-amine | 595.1 |
| 137 | 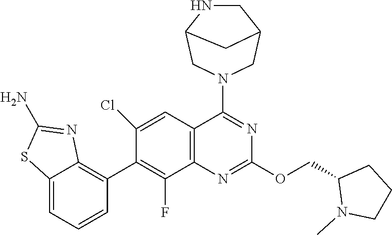 [SMILES: CN1CCC[C@H]1COc1nc(N2CC3CNC(C3)C2)c2cc(Cl)c(-c3cccc4sc(N)nc34)c(F)c2n1] | 4-(4-(3,6-diazabicyclo- [3.2.1]octan-3-yl)-6-chloro- 8-fluoro-2-(((S)-1-methyl- pyrrolidin-2-yl)methoxy)- quinazolin-7-yl)benzo[d]- thiazol-2-amine | 554.3 |
| 138 | 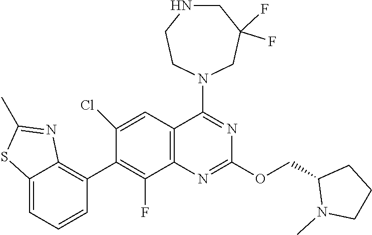 [SMILES: Cc1nc2c(-c3c(Cl)cc4c(N5CCNCC(F)(F)C5)nc(OC[C@@H]5CCCN5C)nc4c3F)cccc2s1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-2-methylbenzo[d]thiazole | 577.2 |
| 139 | 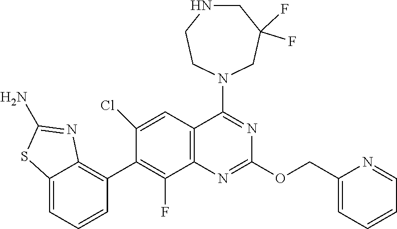 [SMILES: Nc1nc2c(-c3c(Cl)cc4c(N5CCNCC(F)(F)C5)nc(OCc5ccccn5)nc4c3F)cccc2s1] | 4-(6-chloro-4-(6,6-difluoro- 1,4-diazepan-1-yl)-8-fluoro- 2-(pyridin-2-ylmethoxy)- quinazolin-7-yl)benzo[d]- thiazol-2-amine | 572.1 |
| 140 | 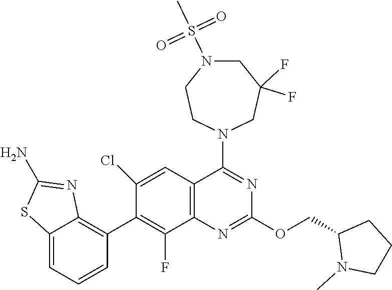 [SMILES: CN1CCC[C@H]1COc1nc(N2CCN(S(C)(=O)=O)CC(F)(F)C2)c2cc(Cl)c(-c3cccc4sc(N)nc34)c(F)c2n1] | 4-(6-chloro-4-(6,6-difluoro- 4-(methylsulfonyl)-1,4- diazepan-1-yl)-8-fluoro-2- (((S)-1-methylpyrrolidin-2- yl)methoxy)quinazolin-7-yl)- benzo[d]thiazol-2-amine | 656.1 |
| 141 | 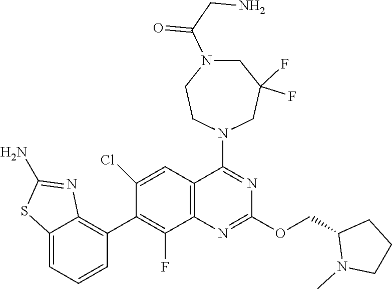 [SMILES: CN1CCC[C@H]1COc1nc(N2CCN(C(=O)CN)CC(F)(F)C2)c2cc(Cl)c(-c3cccc4sc(N)nc34)c(F)c2n1] | 2-amino-1-(4-(7-(2-amino- benzo[d]thiazol-4-yl)-6- chloro-8-fluoro-2-(((S)-1- methylpyrrolidin-2-yl)meth- oxy)quinazolin-4-yl)-6,6- difluoro-1,4-diazepan-1-yl)- ethan-1-one | 635.2 |
| 142 | 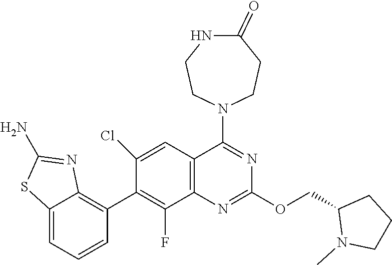 [SMILES: CN1CCC[C@H]1COc1nc(N2CCNC(=O)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)-1,4- diazepan-5-one | 556.3 |
| 143 | 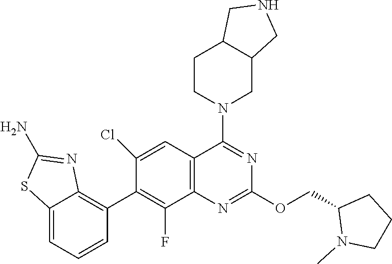 [SMILES: CN1CCC[C@H]1COc1nc(N2CCC3CNCC3C2)c2cc(Cl)c(-c3cccc4sc(N)nc34)c(F)c2n1] | 4-(6-chloro-8-fluoro-2-(((S)- 1-methylpyrrolidin-2-yl)- methoxy)-4-(octahydro-5H- pyrrolo[3,4-c]pyridin-5-yl)- quinazolin-7-yl)benzo[d]- thiazol-2-amine | 568.2 |
| 144 | 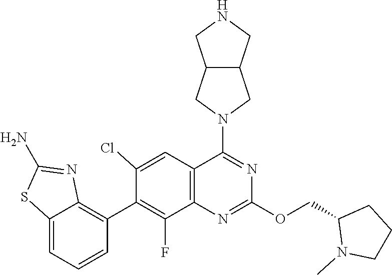 [SMILES: CN1CCC[C@H]1COc1nc(N2CC3CNCC3C2)c2cc(Cl)c(-c3cccc4sc(N)nc34)c(F)c2n1] | 4-(6-chloro-8-fluoro-4- (hexahydropyrrolo[3,4-c]- pyrrol-2(1H)-yl)-2-(((S)-1- methylpyrrolidin-2-yl)meth- oxy)quinazolin-7-yl)benzo- [d]thiazol-2-amine | 554.2 |
| 145 | 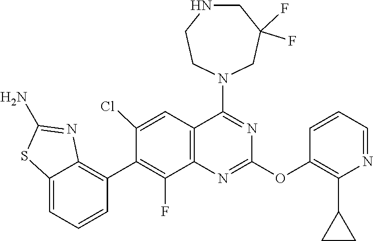 [SMILES: Nc1nc2c(-c3c(Cl)cc4c(N5CCNCC(F)(F)C5)nc(Oc5cccnc5C5CC5)nc4c3F)cccc2s1] | 4-(6-chloro-2-((2-cycloprop- ylpyridin-3-yl)oxy)-4-(6,6- difluoro-1,4-diazepan-1-yl)- 8-fluoroquinazolin-7-yl)- benzo[d]thiazol-2-amine | 598.0 |
| 146 | 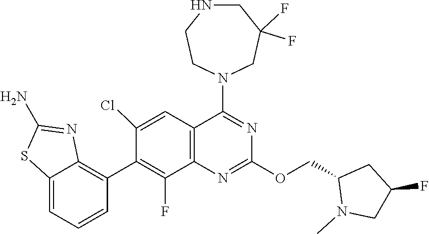 [SMILES: CN1C[C@H](F)C[C@H]1COc1nc(N2CCNCC(F)(F)C2)c2cc(Cl)c(-c3cccc4sc(N)nc34)c(F)c2n1] | 4-(6-chloro-4-(6,6-difluoro- 1,4-diazepan-1-yl)-8-fluoro- 2-(((2S,4R)-4-fluoro-1- methylpyrrolidin-2-yl)meth- oxy)quinazolin-7-yl)benzo- [d]thiazol-2-amine | 596.3 |
| 147 | 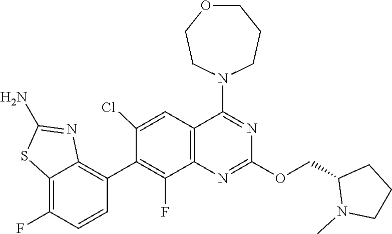 [SMILES: CN1CCC[C@H]1COc1nc(N2CCCOCC2)c2cc(Cl)c(-c3ccc(F)c4sc(N)nc34)c(F)c2n1] | 4-(6-chloro-8-fluoro-2-(((S)- 1-methylpyrrolidin-2-yl)- methoxy)-4-(1,4-oxazepan- 4-yl)quinazolin-7-yl)-7- fluorobenzo[d]thiazol-2- amine | 561.3 |
| 148 | 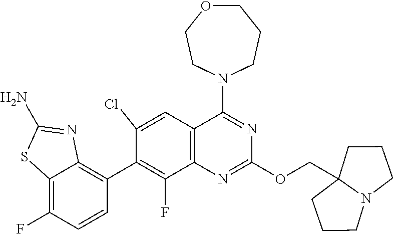 [SMILES: Nc1nc2c(-c3c(Cl)cc4c(N5CCCOCC5)nc(OCC56CCCN5CCC6)nc4c3F)ccc(F)c2s1] | 4-(6-chloro-8-fluoro-4-(1,4- oxazepan-4-yl)-2-((tetra- hydro-1H-pyrrolizin-7a(5H)- yl)methoxy)quinazolin-7-yl)- 7-fluorobenzo[d]thiazol-2- amine | 587.3 |
| 149 | 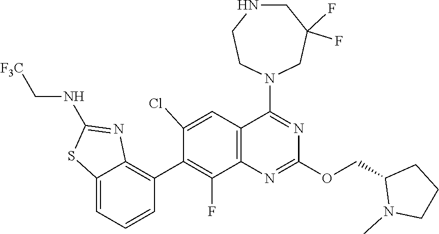 [SMILES: CN1CCC[C@H]1COc1nc(N2CCNCC(F)(F)C2)c2cc(Cl)c(-c3cccc4sc(NCC(F)(F)F)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-N-(2,2,2-trifluoroethyl)- benzo[d]thiazol-2-amine | 660.2 |
| 150 | 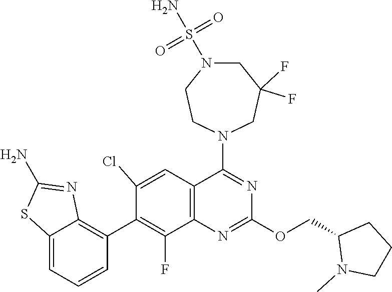 [SMILES: CN1CCC[C@H]1COc1nc(N2CCN(S(N)(=O)=O)CC(F)(F)C2)c2cc(Cl)c(-c3cccc4sc(N)nc34)c(F)c2n1] | 4-(7-(2-aminobenzo[d]- thiazol-4-yl)-6-chloro-8- fluoro-2-(((S)-1-methyl- pyrrolidin-2-yl)methoxy)- quinazolin-4-yl)-6,6- difluoro-1,4-diazepane-1- sulfonamide | 657.1 |
| 151 | 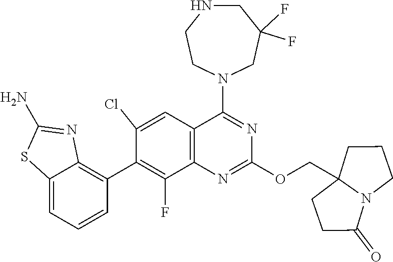 [SMILES: Nc1nc2c(-c3c(Cl)cc4c(N5CCNCC(F)(F)C5)nc(OCC56CCCN5C(=O)CC6)nc4c3F)cccc2s1] | 7a-(((7-(2-aminobenzo[d]- thiazol-4-yl)-6-chloro-4- (6,6-difluoro-1,4-diazepan- 1-yl)-8-fluoroquinazolin-2- yl)oxy)methyl)hexahydro- 3H-pyrrolizin-3-one | 618.3 |
| 152 | 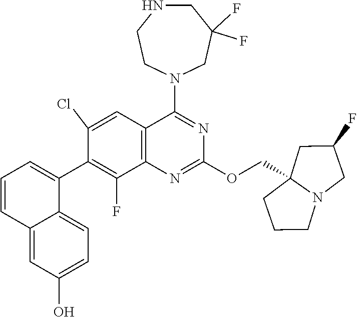 [SMILES: Oc1ccc2c(-c3c(Cl)cc4c(N5CCNCC(F)(F)C5)nc(OC[C@@]56CCCN5C[C@H](F)C6)nc4c3F)cccc2c1] | 4-(6-chloro-4-(6,6-difluoro- 1,4-diazepan-1-yl)-8-fluoro- 2-(((2R,7aS)-2-fluorotetra- hydro-1H-pyrrolizin-7a(5H)- yl)methoxy)quinazolin-7- yl)naphthalen-2-ol | 616.3 |
| 153 | 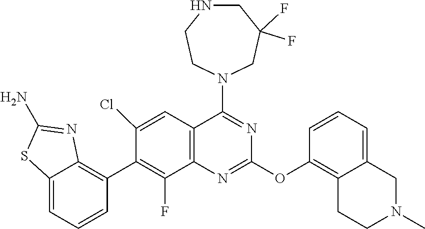 [SMILES: CN1CCc2c(cccc2Oc2nc(N3CCNCC(F)(F)C3)c3cc(Cl)c(-c4cccc5sc(N)nc45)c(F)c3n2)C1] | 4-(6-chloro-4-(6,6-difluoro- 1,4-diazepan-1-yl)-8-fluoro- 2-((2-methyl-1,2,3,4-tetra- hydroisoquinolin-5-yl)oxy)- quinazolin-7-yl)benzo[d]- thiazol-2-amine | 626.4 |
| 154 | 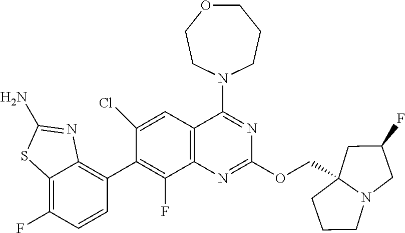 [SMILES: Nc1nc2c(-c3c(Cl)cc4c(N5CCCOCC5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin- 7a(5H)-yl)methoxy)-4- (1,4-oxazepan-4-yl)quin- azolin-7-yl)-7-fluoro- benzo[d]-thiazol-2-amine | 605.2 |
| 155 | 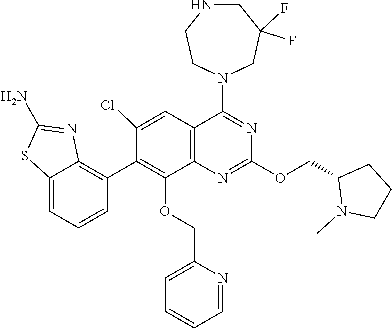 [SMILES: CN1CCC[C@H]1COc1nc(N2CCNCC(F)(F)C2)c2cc(Cl)c(-c3cccc4sc(N)nc34)c(OCc3ccccn3)c2n1] | 4-(6-chloro-4-(6,6-difluoro- 1,4-diazepan-1-yl)-2-(((S)- 1-methylpyrrolidin-2-yl)- methoxy)-8-(pyridin-2-yl- methoxy)quinazolin-7-yl)- benzo[d]thiazol-2-amine | 667.4 |
| 156 | 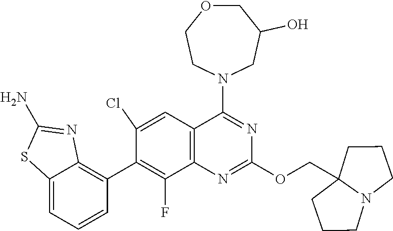 [SMILES: Nc1nc2c(-c3c(Cl)cc4c(N5CCOCC(O)C5)nc(OCC56CCCN5CCC6)nc4c3F)cccc2s1] | 4-(7-(2-aminobenzo[d]- thiazol-4-yl)-6-chloro-8- fluoro-2-((tetrahydro-1H- pyrrolizin-7a(5H)-yl)- methoxy)quinazolin-4- yl)-1,4-oxazepan-6-ol | 585.2 |
| 157 | 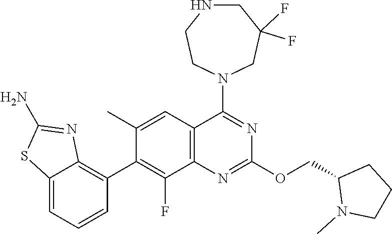 [SMILES: Cc1cc2c(N3CCNCC(F)(F)C3)nc(OC[C@@H]3CCCN3C)nc2c(F)c1-c1cccc2sc(N)nc12] | 4-(4-(6,6-difluoro-1,4- diazepan-1-yl)-8-fluoro-6- methyl-2-(((S)-1-methyl- pyrrolidin-2-yl)methoxy)- quinazolin-7-yl)benzo[d]- thiazol-2-amine | 558.4 |
| 158 | 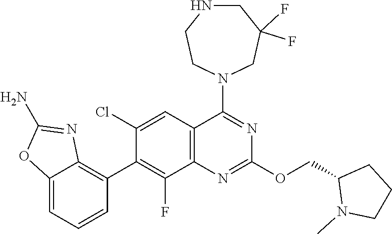 [SMILES: CN1CCC[C@H]1COc1nc(N2CCNCC(F)(F)C2)c2cc(Cl)c(-c3cccc4oc(N)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]oxazol-2-amine | 562.3 |
| 159 | 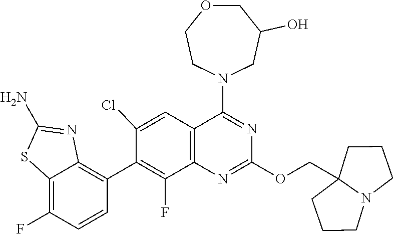 [SMILES: Nc1nc2c(-c3c(Cl)cc4c(N5CCOCC(O)C5)nc(OCC56CCCN5CCC6)nc4c3F)ccc(F)c2s1] | 4-(7-(2-amino-7-fluoro- benzo[d]thiazol-4-yl)-6- chloro-8-fluoro-2-((tetra- hydro-1H-pyrrolizin-7a(5H)- yl)methoxy)quinazolin-4- yl)-1,4-oxazepan-6-ol | 603.7 |
| 160 | 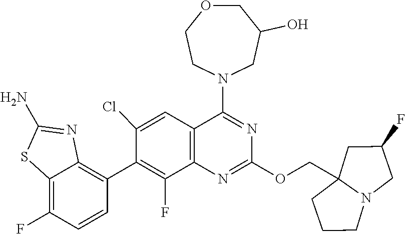 [SMILES: Nc1nc2c(-c3c(Cl)cc4c(N5CCOCC(O)C5)nc(OCC56CCCN5C[C@H](F)C6)nc4c3F)ccc(F)c2s1] | 4-(7-(2-amino-7-fluoro- benzo[d]thiazol-4-yl)-6- chloro-8-fluoro-2-(((2R)-2- fluorotetrahydro-1H-pyrrol- izin-7a(5H)-yl)methoxy)- quinazolin-4-yl)-1,4- oxazepan-6-ol | 621.5 |
| 161 | 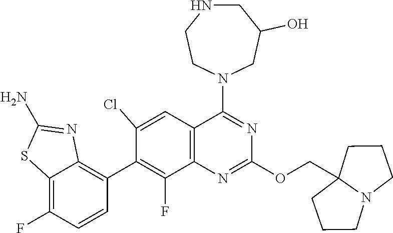 [SMILES: Nc1nc2c(-c3c(Cl)cc4c(N5CCNCC(O)C5)nc(OCC56CCCN5CCC6)nc4c3F)ccc(F)c2s1] | 1-(7-(2-amino-7-fluoro- benzo[d]thiazol-4-yl)-6- chloro-8-fluoro-2-((tetra- hydro-1H-pyrrolizin-7a(5H)- yl)methoxy)quinazolin-4- yl)-1,4-diazepan-6-ol | 602.8 |
| 162 | 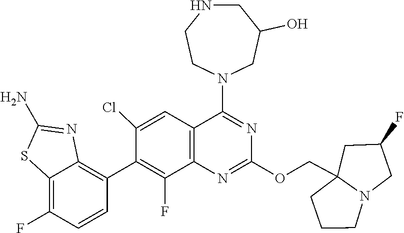 [SMILES: Nc1nc2c(-c3c(Cl)cc4c(N5CCNCC(O)C5)nc(OCC56CCCN5C[C@H](F)C6)nc4c3F)ccc(F)c2s1] | 1-(7-(2-amino-7-fluoro- benzo[d]thiazol-4-yl)-6- chloro-8-fluoro-2-(((2R)-2- fluorotetrahydro-1H-pyrrol- izin-7a(5H)-yl)methoxy)- quinazolin-4-yl)-1,4- diazepan-6-ol | 620.8 |
| 163 | 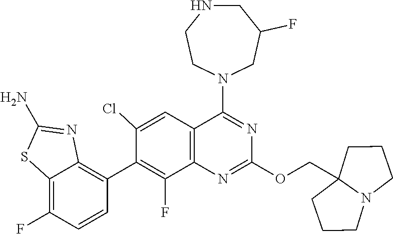 [SMILES: Nc1nc2c(-c3c(Cl)cc4c(N5CCNCC(F)C5)nc(OCC56CCCN5CCC6)nc4c3F)ccc(F)c2s1] | 4-(6-chloro-8-fluoro-4-(6- fluoro-1,4-diazepan-1-yl)-2- ((tetrahydro-1H-pyrrolizin- 7a(5H)-yl)methoxy)quin- azolin-7-yl)-7-fluorobenzo- [d]thiazol-2-amine | 604.4 |
| 164 | 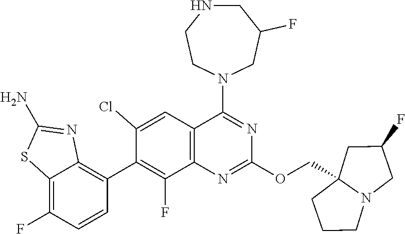 [SMILES: Nc1nc2c(-c3c(Cl)cc4c(N5CCNCC(F)C5)nc(OC[C@@]56CCCN5C[C@H](F)C6)nc4c3F)ccc(F)c2s1] | 4-(6-chloro-8-fluoro-4-(6- fluoro-1,4-diazepan-1-yl)-2- (((2R,7aS)-2-fluorotetra- hydro-1H-pyrrolizin-7a(5H)- yl)methoxy)quinazolin-7- yl)-7-fluorobenzo[d]thiazol- 2-amine | 622.6 |
| 165 | 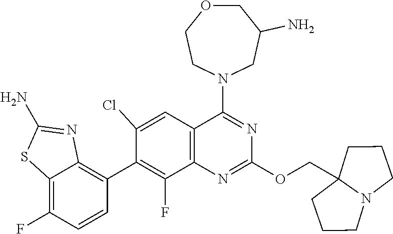 [SMILES: Nc1nc2c(-c3c(Cl)cc4c(N5CCOCC(N)C5)nc(OCC56CCCN5CCC6)nc4c3F)ccc(F)c2s1] | 4-(7-(2-amino-7-fluoro- benzo[d]thiazol-4-yl)-6- chloro-8-fluoro-2-((tetra- hydro-1H-pyrrolizin-7a(5H)- yl)methoxy)quinazolin-4- yl)-1,4-oxazepan-6-amine | 602.3 |
| 166 | 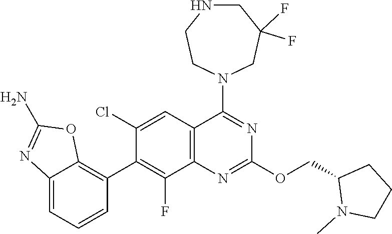 [SMILES: CN1CCC[C@H]1COc1nc(N2CCNCC(F)(F)C2)c2cc(Cl)c(-c3cccc4nc(N)oc34)c(F)c2n1] | 7-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]oxazol-2-amine | 562.4 |
| 167 | 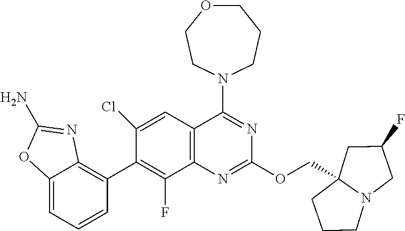 [SMILES: Nc1nc2c(-c3c(Cl)cc4c(N5CCCOCC5)nc(OC[C@@]56CCCN5C[C@H](F)C6)nc4c3F)cccc2o1] | 4-(6-chloro-8-fluoro-2- (((2R,7aS)-2-fluorotetra- hydro-1H-pyrrolizin- 7a(5H)-yl)methoxy)-4-(1,4- oxazepan-4-yl)quinazolin-7- yl)benzo[d]thiazol-2-amine | 571.5 |
| 168 | 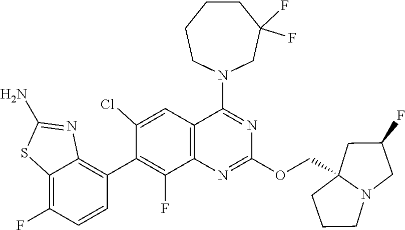 [SMILES: Nc1nc2c(-c3c(Cl)cc4c(N5CCCCC(F)(F)C5)nc(OC[C@@]56CCCN5C[C@H](F)C6)nc4c3F)ccc(F)c2s1] | 4-(6-chloro-4-(3,3-difluoro- azepan-1-yl)-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 639.8 |
| 169 | 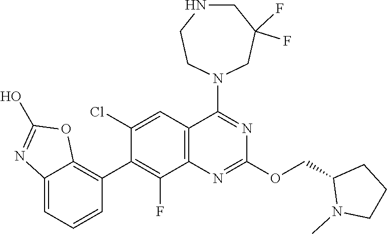 [SMILES: CN1CCC[C@H]1COc1nc(N2CCNCC(F)(F)C2)c2cc(Cl)c(-c3cccc4nc(O)oc34)c(F)c2n1] | 7-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]oxazol-2-ol | 563.4 |
| 170 | 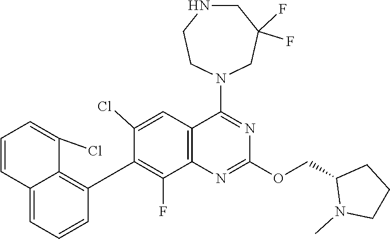 [SMILES: CN1CCC[C@H]1COc1nc(N2CCNCC(F)(F)C2)c2cc(Cl)c(-c3cccc4cccc(Cl)c34)c(F)c2n1] | 6-chloro-7-(8-chloronaph- thalen-1-yl)-4-(6,6-difluoro- 1,4-diazepan-1-yl)-8-fluoro- 2-(((S)-1-methylpyrrolidin- 2-yl)methoxy)quinazoline | 590.5 |
| 171 | 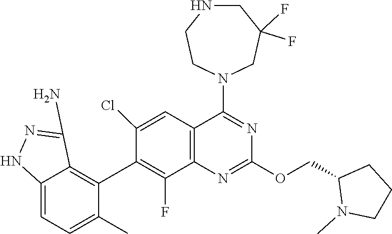 [SMILES: Cc1ccc2[nH]nc(N)c2c1-c1c(Cl)cc2c(N3CCNCC(F)(F)C3)nc(OC[C@@H]3CCCN3C)nc2c1F] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-5-methyl-1H-indazol-3- amine | 575.5 |
| 172 | 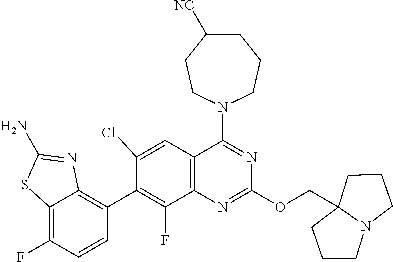 [SMILES: N#CC1CCCN(c2nc(OCC34CCCN3CCC4)nc3c(F)c(-c4ccc(F)c5sc(N)nc45)c(Cl)cc23)CC1] | 1-(7-(2-amino-7-fluoro- benzo[d]thiazol-4-yl)-6- chloro-8-fluoro-2-((tetra- hydro-1H-pyrrolizin- 7a(5H)-yl)methoxy)- quinazolin-4-yl)azepane- 4-carbonitrile | 610.7 |
| 173 | 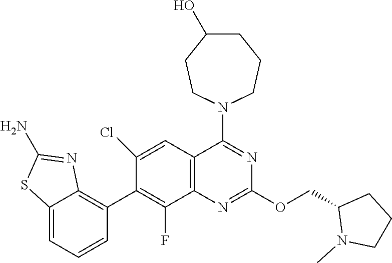 [SMILES: CN1CCC[C@H]1COc1nc(N2CCCC(O)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)azepan-4- ol | 557.2 |
| 174 | 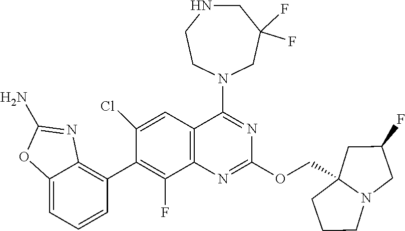 [SMILES: Nc1nc2c(-c3c(Cl)cc4c(N5CCNCC(F)(F)C5)nc(OC[C@@]56CCCN5C[C@H](F)C6)nc4c3F)cccc2o1] | 4-(6-chloro-4-(6,6-difluoro- 1,4-diazepan-1-yl)-8-fluoro- 2-(((2R,7aS)-2-fluorotetra- hydro-1H-pyrrolizin-7a(5H)- yl)methoxy)quinazolin-7- yl)benzo[d]oxazol-2-amine | 606.4 |
| 175 | 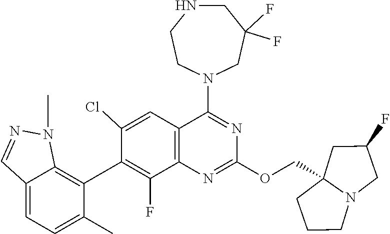 [SMILES: Cc1ccc2cnn(C)c2c1-c1c(Cl)cc2c(N3CCNCC(F)(F)C3)nc(OC[C@@]34CCCN3C[C@H](F)C4)nc2c1F] | 6-chloro-4-(6,6-difluoro- 1,4-diazepan-1-yl)-7-(1,6- dimethyl-1H-indazol-7-yl)- 8-fluoro-2-(((2R,7aS)-2- fluorotetrahydro-1H- pyrrolizin-7a(5H)-yl)meth- oxy)quinazoline | 618.5 |
| 176 | 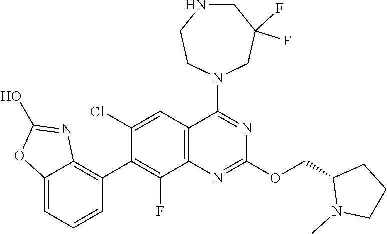 [SMILES: CN1CCC[C@H]1COc1nc(N2CCNCC(F)(F)C2)c2cc(Cl)c(-c3cccc4oc(O)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin- 7-yl)benzo[d]oxazol-2-ol | 563.5 |
| 177 | 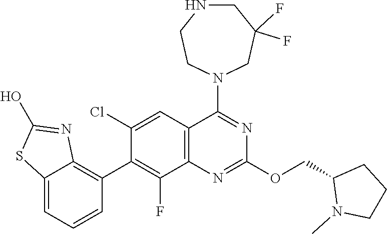 [SMILES: CN1CCC[C@H]1COc1nc(N2CCNCC(F)(F)C2)c2cc(Cl)c(-c3cccc4sc(O)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]thiazol-2-ol | 579.4 |
| 178 | 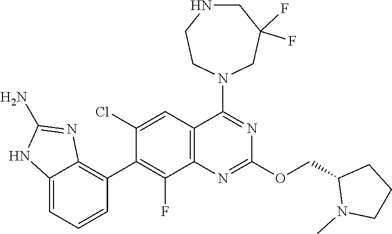 [SMILES: CN1CCC[C@H]1COc1nc(N2CCNCC(F)(F)C2)c2cc(Cl)c(-c3cccc4[nH]c(N)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-1H-benzo[d]imidazol-2- amine | 561.4 |
| 179 | 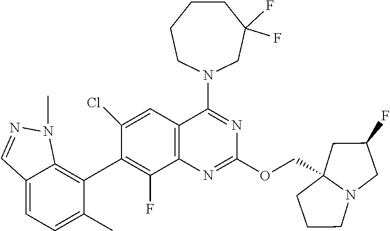 [SMILES: Cc1ccc2cnn(C)c2c1-c1c(Cl)cc2c(N3CCCCC(F)(F)C3)nc(OC[C@@]34CCCN3C[C@H](F)C4)nc2c1F] | 6-chloro-4-(3,3-difluoro- azepan-1-yl)-7-(1,6- dimethyl-1H-indazol-7- yl)-8-fluoro-2-(((2R,7aS)- 2-fluoro-tetrahydro-1H- pyrrolizin-7a(5H)-yl)- methoxy)quinazoline | 617.5 |
| 180 | 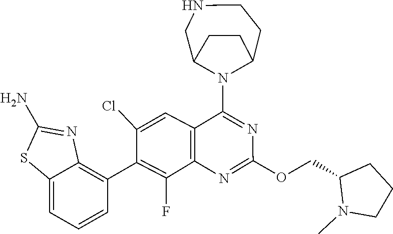 [SMILES: CN1CCC[C@H]1COc1nc(N2C3CCNCC2CC3)c2cc(Cl)c(-c3cccc4sc(N)nc34)c(F)c2n1] | 4-(4-(3,9-diazabicyclo- [4.2.1]nonan-9-yl)-6-chloro- 8-fluoro-2-(((S)-1-methyl- pyrrolidin-2-yl)methoxy)- quinazolin-7-yl)benzo[d]- thiazol-2-amine | 568.3 |
| 181 | 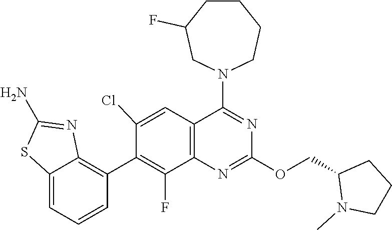 [SMILES: CN1CCC[C@H]1COc1nc(N2CCCCC(F)C2)c2cc(Cl)c(-c3cccc4sc(N)nc34)c(F)c2n1] | 4-(6-chloro-8-fluoro-4-(3- fluoroazepan-1-yl)-2-(((S)- 1-methylpyrrolidin-2-yl)- methoxy)quinazolin-7-yl)- benzo[d]thiazol-2-amine | 559.2 |
| 182 | 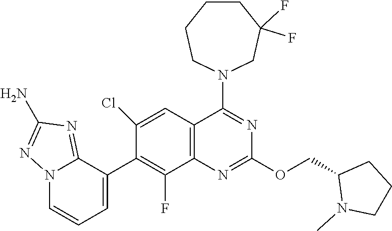 [SMILES: CN1CCC[C@H]1COc1nc(N2CCCCC(F)(F)C2)c2cc(Cl)c(-c3cccn4nc(N)nc34)c(F)c2n1] | (S)-8-(6-chloro-4-(3,3- difluoroazepan-1-yl)-8- fluoro-2-((1-methylpyrrol- idin-2-yl)methoxy)quin- azolin-7-yl)[1,2,4]triazolo- [1,5-a]pyridin-2-amine | 561.5 |
| 183 | 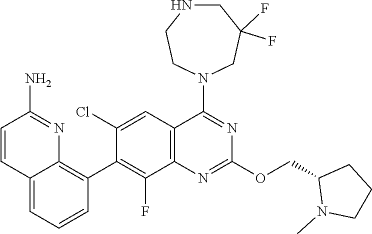 [SMILES: CN1CCC[C@H]1COc1nc(N2CCNCC(F)(F)C2)c2cc(Cl)c(-c3cccc4ccc(N)nc34)c(F)c2n1] | 8-(6-chloro-4-(6,6-difluoro- 1,4-diazepan-1-yl)-8-fluoro- 2-(((S)-1-methylpyrrolidin- 2-yl)methoxy)quinazolin-7- yl)quinolin-2-amine | 572.5 |
| 184 | 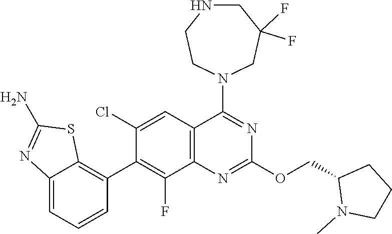 [SMILES: CN1CCC[C@H]1COc1nc(N2CCNCC(F)(F)C2)c2cc(Cl)c(-c3cccc4nc(N)sc34)c(F)c2n1] | 7-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]thiazol-2-amine | 578.5 |
| 185 | 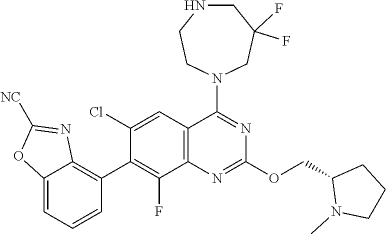 [SMILES: CN1CCC[C@H]1COc1nc(N2CCNCC(F)(F)C2)c2cc(Cl)c(-c3cccc4oc(C#N)nc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin-7- yl)benzo[d]oxazole-2- carbonitrile | 572.5 |
| 186 | 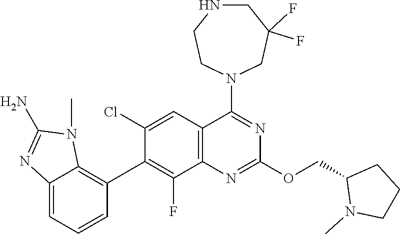 [SMILES: CN1CCC[C@H]1COc1nc(N2CCNCC(F)(F)C2)c2cc(Cl)c(-c3cccc4nc(N)n(C)c34)c(F)c2n1] | 7-(6-chloro-4-(6,6-difluoro- 1,4-diazepan-1-yl)-8-fluoro- 2-(((S)-1-methylpyrrolidin- 2-yl)methoxy)quinazolin-7- yl)-1-methyl-1H-benzo[d]- imidazol-2-amine | 575.5 |
| 187 | 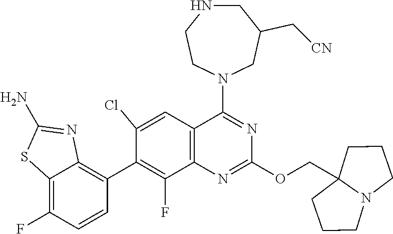 [SMILES: N#CCC1CNCCN(c2nc(OCC34CCCN3CCC4)nc3c(F)c(-c4ccc(F)c5sc(N)nc45)c(Cl)cc23)C1] | 2-(1-(7-(2-amino-7-fluoro- benzo[d]thiazol-4-yl)-6- chloro-8-fluoro-2-((tetra- hydro-1H-pyrrolizin- 7a(5H)-yl)methoxy)quin- azolin-4-yl)-1,4-diazepan- 6-yl)acetonitrile | 625.2 |
| 188 | 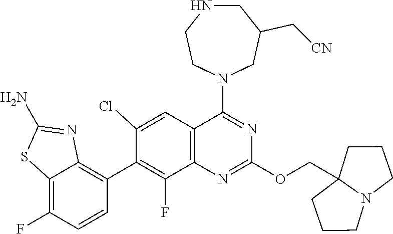 [SMILES: N#CCC1CNCCN(c2nc(OCC34CCCN3CCC4)nc3c(F)c(-c4ccc(F)c5sc(N)nc45)c(Cl)cc23)C1] | 2-(1-(7-(2-amino-7-fluoro- benzo[d]thiazol-4-yl)-6- chloro-8-fluoro-2-((tetra- hydro-1H-pyrrolizin- 7a(5H)-yl)methoxy)quin- azolin-4-yl)-1,4-diazepan- 6-yl)acetonitrile | 625.2 |
| 189 | 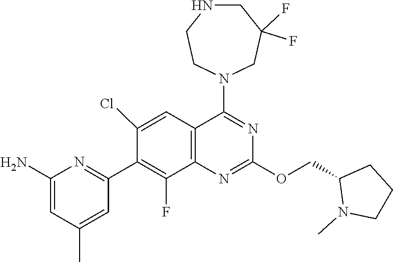 [SMILES: Cc1cc(N)nc(-c2c(Cl)cc3c(N4CCNCC(F)(F)C4)nc(OC[C@@H]4CCCN4C)nc3c2F)c1] | (S)-6-(6-chloro-4-(6,6- difluoro-1,4-diazepan-1- yl)-8-fluoro-2-((1-methyl- pyrrolidin-2-yl)methoxy)- quinazolin-7-yl)-4-methyl- pyridin-2-amine | 536.5 |
| 190 | 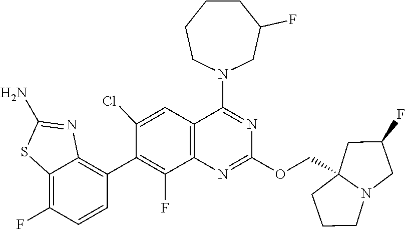 [SMILES: Nc1nc2c(-c3c(Cl)cc4c(N5CCCCC(F)C5)nc(OC[C@@]56CCCN5C[C@H](F)C6)nc4c3F)ccc(F)c2s1] | 4-(6-chloro-8-fluoro-4-(3- fluoroazepan-1-yl)-2- (((2R,7aS)-2-fluorotetra- hydro-1H-pyrrolizin- 7a(5H)-yl)methoxy)quin- azolin-7-yl)-7-fluorobenzo- [d]thiazol-2-amine | 621.2 |
| 191 | 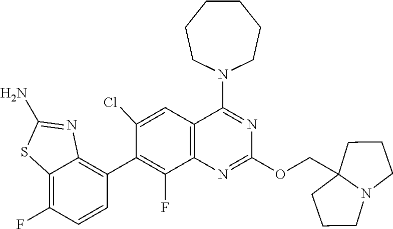 [SMILES: Nc1nc2c(-c3c(Cl)cc4c(N5CCCCCC5)nc(OCC56CCCN5CCC6)nc4c3F)ccc(F)c2s1] | 4-(4-(azepan-1-yl)-6-chloro- 8-fluoro-2-((tetrahydro-1H- pyrrolizin-7a(5H)-yl)meth- oxy)quinazolin-7-yl)-7- fluorobenzo[d]thiazol-2- amine | 585.6 |
| 192 | 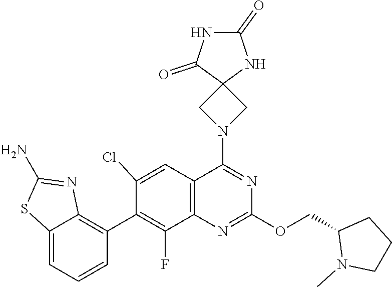 [SMILES: CN1CCC[C@H]1COc1nc(N2CC3(C2)NC(=O)NC3=O)c2cc(Cl)c(-c3cccc4sc(N)nc34)c(F)c2n1] | 2-(7-(2-aminobenzo[d]- thiazol-4-yl)-6-chloro-8- fluoro-2-(((S)-1-methyl- pyrrolidin-2-yl)methoxy)- quinazolin-4-yl)-2,5,7- triazaspiro[3.4]octane-6,8- dione | 583.1 |
| 193 | 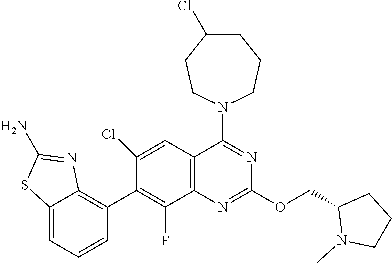 [SMILES: CN1CCC[C@H]1COc1nc(N2CCCC(Cl)CC2)c2cc(Cl)c(-c3cccc4sc(N)nc34)c(F)c2n1] | 4-(6-chloro-4-(4-chloro- azepan-1-yl)-8-fluoro-2- (((S)-1-methylpyrrolidin- 2-yl)methoxy)quinazolin- 7-yl)benzo[d]thiazol-2- amine | 575.1 |
| 194 | 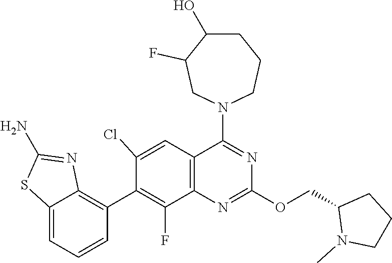 [SMILES: CN1CCC[C@H]1COc1nc(N2CCCC(O)C(F)C2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)-3-fluoro- azepan-4-ol | 575.0 |
| 196 | 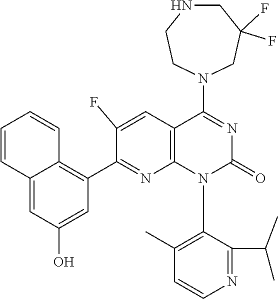 [SMILES: Cc1ccnc(C(C)C)c1-n1c(=O)nc(N2CCNCC(F)(F)C2)c2cc(F)c(-c3cc(O)cc4ccccc34)nc21] | 4-(6,6-difluoro-1,4- diazepan-1-yl)-6-fluoro-7- (3-hydroxynaphthalen-1- yl)-1-(2-isopropyl-4-methyl- pyridin-3-yl)pyrido[2,3-d]- pyrimidin-2(1H)-one | 575.3 |
| 204 | 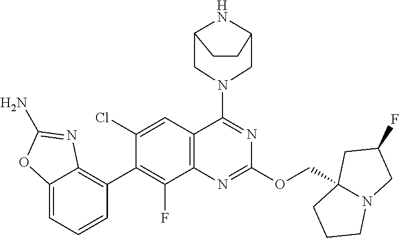 [SMILES: Nc1nc2c(-c3c(Cl)cc4c(N5CC6CCC(C5)N6)nc(OC[C@@]56CCCN5C[C@H](F)C6)nc4c3F)cccc2o1] | 4-(4-((1R,5S)-3,8-diaza- bicyclo[3.2.1]octan-3-yl)- 6-chloro-8-fluoro-2- (((2R,7aS)-2-fluorotetra- hydro-1H-pyrrolizin- 7a(5H)-yl)methoxy)quin- azolin-7-yl)benzo[d]- oxazol-2-amine | 582.5 |
| 205 | 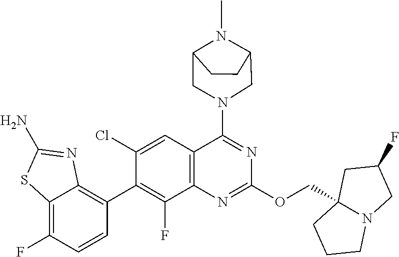 [SMILES: CN1C2CCC1CN(c1nc(OC[C@@]34CCCN3C[C@H](F)C4)nc3c(F)c(-c4ccc(F)c5sc(N)nc45)c(Cl)cc13)C2] | 4-(6-chloro-8-fluoro-2- (((2R,7aS)-2-fluorotetra- hydro-1H-pyrrolizin- 7a(5H)-yl)methoxy)-4- ((1R,5S)-8-methyl-3,8- diazabicyclo[3.2.1]octan- 3-yl)quinazolin-7-yl)-7- fluorobenzo[d]thiazol-2- amine | 630.5 |
| 206 | 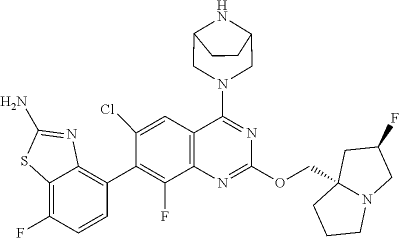 [SMILES: Nc1nc2c(-c3c(Cl)cc4c(N5CC6CCC(C5)N6)nc(OC[C@@]56CCCN5C[C@H](F)C6)nc4c3F)ccc(F)c2s1] | 4-(4-((1R,5S)-3,8-diaza- bicyclo[3.2.1]octan-3-yl)- 6-chloro-8-fluoro-2- (((2R,7aS)-2-fluorotetra- hydro-1H-pyrrolizin-7a(5H)- yl)methoxy)quinazolin-7-yl)- 7-fluorobenzo[d]thiazol-2- amine | 616.5 |
| 207 | 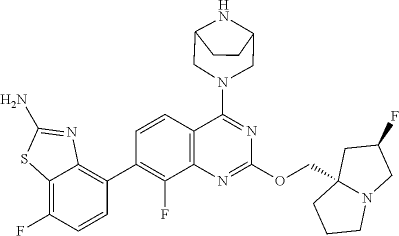 [SMILES: Nc1nc2c(-c3ccc4c(N5CC6CCC(C5)N6)nc(OC[C@@]56CCCN5C[C@H](F)C6)nc4c3F)ccc(F)c2s1] | 4-(4-((1R,5S)-3,8-diaza- bicyclo[3.2.1]octan-3-yl)-8- fluoro-2-(((2R,7aS)-2-fluoro- tetrahydro-1H-pyrrolizin- 7a(5H)-yl)methoxy)quin- azolin-7-yl)-7-fluorobenzo- [d]thiazol-2-amine | 582.5 |
| 208 | 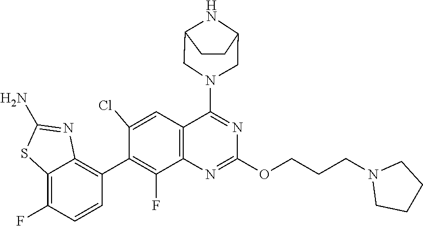 [SMILES: Nc1nc2c(-c3c(Cl)cc4c(N5CC6CCC(C5)N6)nc(OCCCN5CCCC5)nc4c3F)ccc(F)c2s1] | 4-(4-((1R,5S)-3,8-diaza- bicyclo[3.2.1]octan-3-yl)- 6-chloro-8-fluoro-2-(3- (pyrrolidin-1-yl)propoxy)- quinazolin-7-yl)-7-fluoro- benzo[d]thiazol-2-amine | 586.5 |
| 209 | 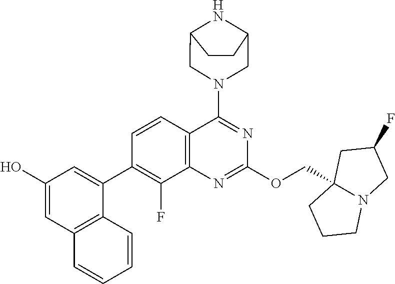 [SMILES: Oc1cc(-c2ccc3c(N4CC5CCC(C4)N5)nc(OC[C@@]45CCCN4C[C@H](F)C5)nc3c2F)c2ccccc2c1] | 4-(4-((1R,5S)-3,8-diaza- bicyclo[3.2.1]octan-3-yl)-8- fluoro-2-(((2R,7aS)-2-fluoro- tetrahydro-1H-pyrrolizin- 7a(5H)-yl)methoxy)quin- azolin-7-yl)naphthalen-2- ol | 558.5 |
| 210 | 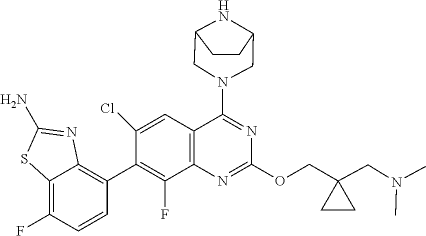 [SMILES: CN(C)CC1(COc2nc(N3CC4CCC(C3)N4)c3cc(Cl)c(-c4ccc(F)c5sc(N)nc45)c(F)c3n2)CC1] | 4-(4-((1R,5S)-3,8-diaza- bicyclo[3.2.1]octan-3-yl)- 6-chloro-2-((1-(dimethyl- amino)methyl)cyclopropyl)- methoxy)-8-fluoroquin- azolin-7-yl)-7-fluorobenzo- [d]thiazol-2-amine | 586.5 |
| 211 | 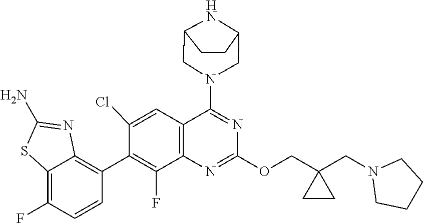 [SMILES: Nc1nc2c(-c3c(Cl)cc4c(N5CC6CCC(C5)N6)nc(OCC5(CN6CCCC6)CC5)nc4c3F)ccc(F)c2s1] | 4-(4-((1R,5S)-3,8-diaza- bicyclo[3.2.1]octan-3-yl)- 6-chloro-8-fluoro-2-((1- (pyrrolidin-1-ylmethyl)- cyclopropyl)methoxy)quin- azolin-7-yl)-7-fluorobenzo- [d]thiazol-2-amine | 612.5 |
| 212 | 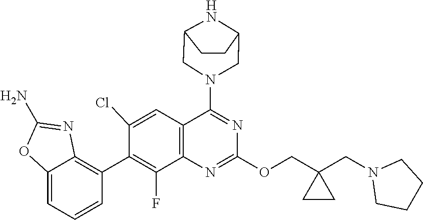 [SMILES: Nc1nc2c(-c3c(Cl)cc4c(N5CC6CCC(C5)N6)nc(OCC5(CN6CCCC6)CC5)nc4c3F)cccc2o1] | 4-(4-((1R,5S)-3,8-diaza- bicyclo[3.2.1]octan-3-yl)- 6-chloro-8-fluoro-2-((1- (pyrrolidin-1-ylmethyl)- cyclopropyl)methoxy)quin- azolin-7-yl)benzo[d]oxazol- 2-amine | 578.5 |
| 213 | 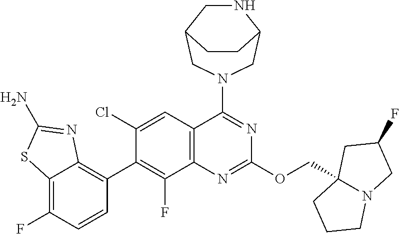 [SMILES: Nc1nc2c(-c3c(Cl)cc4c(N5CC6CCC(C5)NC6)nc(OC[C@@]56CCCN5C[C@H](F)C6)nc4c3F)ccc(F)c2s1] | 4-(4-(3,6-diazabicyclo- [3.2.2]nonan-3-yl)-6-chloro- 8-fluoro-2-(((2R,7aS)-2- fluorotetrahydro-1H- pyrrolizin-7a(5H)-yl)meth- oxy)quinazolin-7-yl)-7- fluorobenzo[d]thiazol-2- amine | 630.5 |
| 301 | 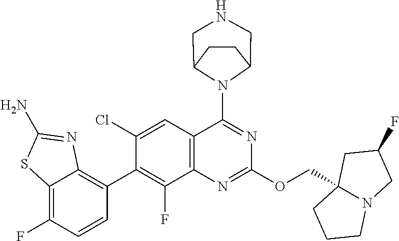 [SMILES: Nc1nc2c(-c3c(Cl)cc4c(N5C6CCC5CNC6)nc(OC[C@@]56CCCN5C[C@H](F)C6)nc4c3F)ccc(F)c2s1] | 4-(4-((1R,5S)-3,8-diaza- bicyclo[3.2.1]octan-8-yl)- 6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 616.6 |
| 302 | 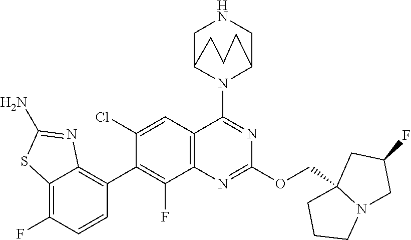 [SMILES: Nc1nc2c(-c3c(Cl)cc4c(N5C6CCCC5CNC6)nc(OC[C@@]56CCCN5C[C@H](F)C6)nc4c3F)ccc(F)c2s1] | 4-(4-(3,9-diazabicyclo- [3.3.1]nonan-9-yl)-6-chloro- 8-fluoro-2-(((2R,7aS)-2- fluorotetrahydro-1H- pyrrolizin-7a(5H)-yl)meth- oxy)quinazolin-7-yl)-7- fluorobenzo[d]thiazol-2- amine | 631.2 |
| 303 | 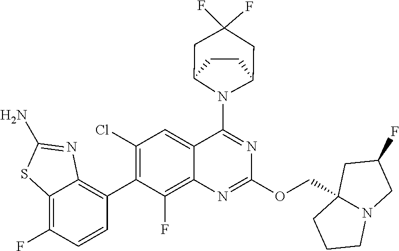 [SMILES: Nc1nc2c(-c3c(Cl)cc4c(N5[C@@H]6CC[C@H]5CC(F)(F)C6)nc(OC[C@@]56CCCN5C[C@H](F)C6)nc4c3F)ccc(F)c2s1] | 4-(6-chloro-4-((1R,5S)-3,3- difluoro-8-azabicyclo[3.2.1]- octan-8-yl)-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 651.2 |
| 304 | 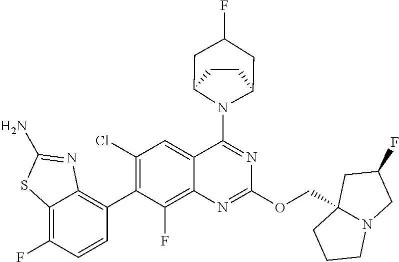 [SMILES: Nc1nc2c(-c3c(Cl)cc4c(N5[C@@H]6CC[C@H]5CC(F)C6)nc(OC[C@@]56CCCN5C[C@H](F)C6)nc4c3F)ccc(F)c2s1] | 4-(6-chloro-8-fluoro-4- ((1R,3S,5S)-3-fluoro-8- azabicyclo[3.2.1]octan-8- yl)-2-(((2R,7aS)-2-fluoro- tetrahydro-1H-pyrrolizin- 7a(5H)-yl)methoxy)quin- azolin-7-yl)-7-fluorobenzo- [d]thiazol-2-amine | 633.1 |
| 305 | 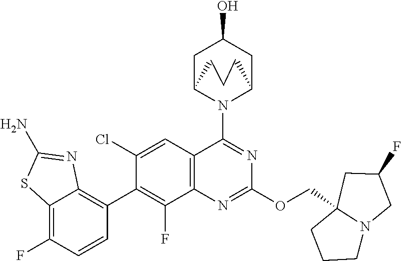 [SMILES: Nc1nc2c(-c3c(Cl)cc4c(N5[C@@H]6CCC[C@H]5C[C@H](O)C6)nc(OC[C@@]56CCCN5C[C@H](F)C6)nc4c3F)ccc(F)c2s1] | (1R,3R,5S)-9-(7-(2-amino- 7-fluorobenzo[d]thiazol-4- yl)-6-chloro-8-fluoro-2- (((2R,7aS)-2-fluorotetra- hydro-1H-pyrrolizin-7a(5H)- yl)methoxy)quinazolin-4- yl)-9-azabicyclo[3.3.1]- nonan-3-ol | 645.2 |
| 306 | 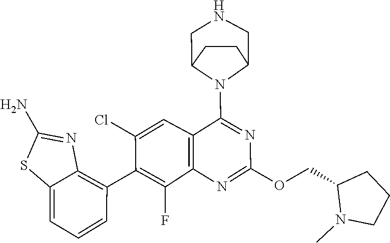 [SMILES: CN1CCC[C@H]1COc1nc(N2C3CCC2CNC3)c2cc(Cl)c(-c3cccc4sc(N)nc34)c(F)c2n1] | 4-(4-((1R,5S)-3,8-diaza- bicyclo[3.2.1]octan-8-yl)- 6-chloro-8-fluoro-2-(((S)-1- methylpyrrolidin-2-yl)meth- oxy)quinazolin-7-yl)benzo- [d]thiazol-2-amine | 554.2 |
| 309 | 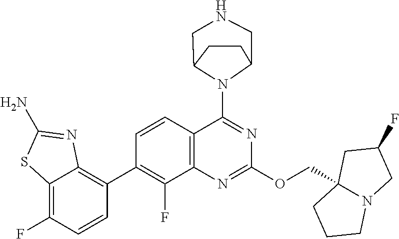 [SMILES: Nc1nc2c(-c3ccc4c(N5C6CCC5CNC6)nc(OC[C@@]56CCCN5C[C@H](F)C6)nc4c3F)ccc(F)c2s1] | 4-(4-((1R,5S)-3,8-diaza- bicyclo[3.2.1]octan-8-yl)-8- fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 582.5 |
| 310 | 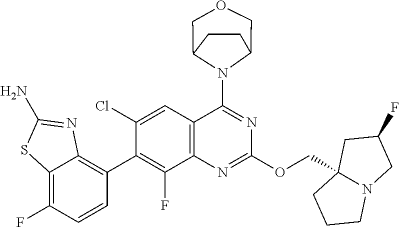 [SMILES: Nc1nc2c(-c3c(Cl)cc4c(N5C6CCC5COC6)nc(OC[C@@]56CCCN5C[C@H](F)C6)nc4c3F)ccc(F)c2s1] | 4-(4-((1R,5S)-3-oxa-8- azabicyclo[3.2.1]octan-8- yl)-6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 617.3 |
| 311 | 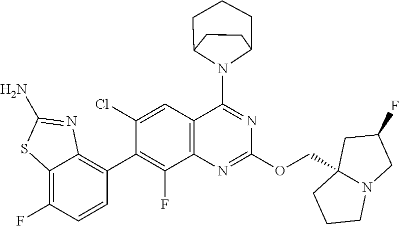 [SMILES: Nc1nc2c(-c3c(Cl)cc4c(N5C6CCCC5CC6)nc(OC[C@@]56CCCN5C[C@H](F)C6)nc4c3F)ccc(F)c2s1] | 4-(4-((1R,5S)-8-azabicyclo- [3.2.1]octan-8-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 615.4 |
| 312 | 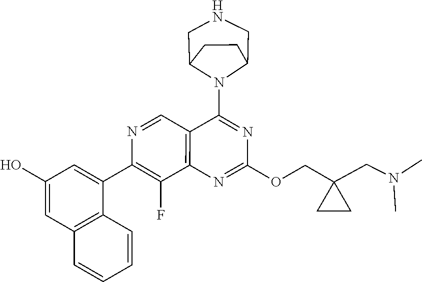 [SMILES: CN(C)CC1(COc2nc(N3C4CCC3CNC4)c3cnc(-c4cc(O)cc5ccccc45)c(F)c3n2)CC1] | 4-(4-((1R,5S)-3,8-diaza- bicyclo[3.2.1]octan-8-yl)-2- ((1-((dimethylamino)meth- yl)cyclopropyl)methoxy)-8- fluoropyrido[4,3-d]pyrim- idin-7-yl)naphthalen-2-ol | 529.5 |
| 401 | 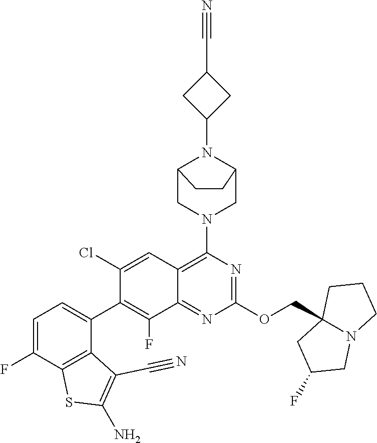 [SMILES: N#Cc1c(N)sc2c(F)ccc(-c3c(Cl)cc4c(N5CC6CCC(C5)N6C5CC(C#N)C5)nc(OC[C@@]56CCCN5C[C@H](F)C6)nc4c3F)c12] | 2-amino-4-(6-chloro-4- ((1R,5S)-8-(3-cyanocyclo- butyl)-3,8-diazabicyclo- [3.2.1]octan-3-yl)-8-fluoro- 2-(((2R,7aS)-2-fluorotetra- hydro-1H-pyrrolizin- 7a(5H)-yl)methoxy)quin- azolin-7-yl)-7-fluorobenzo- [b]thiophene-3-carbonitrile | 719.2 |
| 402 | 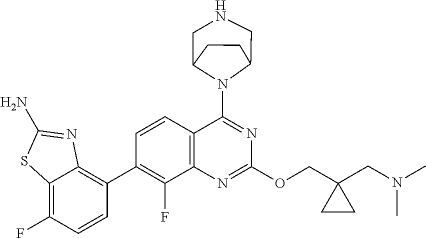 [SMILES: CN(C)CC1(COc2nc(N3C4CCC3CNC4)c3ccc(-c4ccc(F)c5sc(N)nc45)c(F)c3n2)CC1] | 4-(4-((1R,5S)-3,8-diaza- bicyclo[3.2.1]octan-8-yl)-2- ((1-((dimethylamino)meth- yl)cyclopropyl)methoxy)-8- fluoroquinazolin-7-yl)-7- fluorobenzo[d]thiazol-2- amine | 552.4 |
| 403 | 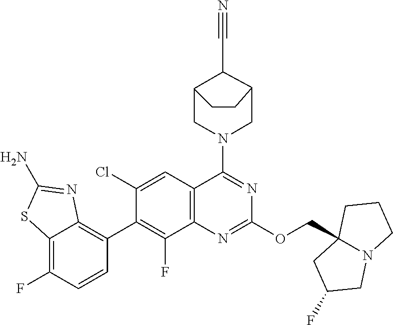 [SMILES: N#CC1C2CCC1CN(c1nc(OC[C@@]34CCCN3C[C@H](F)C4)nc3c(F)c(-c4ccc(F)c5sc(N)nc45)c(Cl)cc13)C2] | (1R,5S,8S)-3-(7-(2-amino- 7-fluorobenzo[d]thiazol-4- yl)-6-chloro-8-fluoro-2- (((2R,7aS)-2-fluorotetra- hydro-1H-pyrrolizin-7a(5H)- yl)methoxy)quinazolin-4- yl)-3-azabicyclo[3.2.1]- octane-8-carbonitrile | 640.4 |
| 404 | 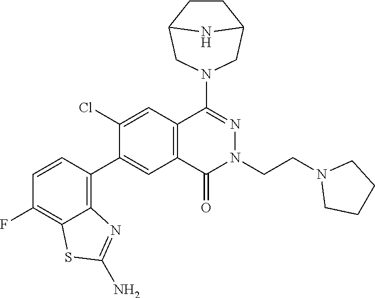 [SMILES: Nc1nc2c(-c3cc4c(=O)n(CCN5CCCC5)nc(N5CC6CCC(C5)N6)c4cc3Cl)ccc(F)c2s1] | 7-(2-amino-7-fluorobenzo- [d]thiazol-4-yl)-4-(3,8- diazabicyclo[3.2.1]octan-3- yl)-6-chloro-2-(2-(pyrrol- idin-1-yl)ethyl)phthalazin- 1(2H)-one | 555.3 |
| 405 | 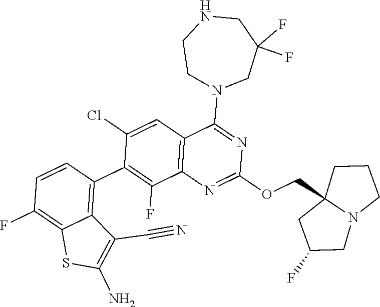 [SMILES: N#Cc1c(N)sc2c(F)ccc(-c3c(Cl)cc4c(N5CCNCC(F)(F)C5)nc(OC[C@@]56CCCN5C[C@H](F)C6)nc4c3F)c12] | 2-amino-4-(6-chloro-4-(6,6- difluoro-1,4-diazepan-1-yl)- 8-fluoro-2-(((2R,7aS)-2- fluorotetrahydro-1H-pyr- rolizin-7a(5H)-yl)methoxy)- quinazolin-7-yl)-7-fluoro- benzo[b]thiophene-3- carbonitrile | 664.3 |
| 406 | 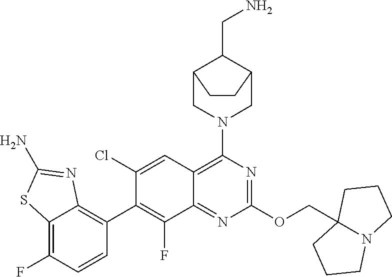 [SMILES: NCC1C2CCC1CN(c1nc(OCC34CCCN3CCC4)nc3c(F)c(-c4ccc(F)c5sc(N)nc45)c(Cl)cc13)C2] | 4-(4-((1R,5S,8r)-8-(amino- methyl)-3-azabicyclo[3.2.1]- octan-3-yl)-6-chloro-8- fluoro-2-((tetrahydro-1H- pyrrolizin-7a(5H)-yl)meth- oxy)quinazolin-7-yl)-7- fluorobenzo[d]thiazol-2- amine | 626.2 |
| 407 | 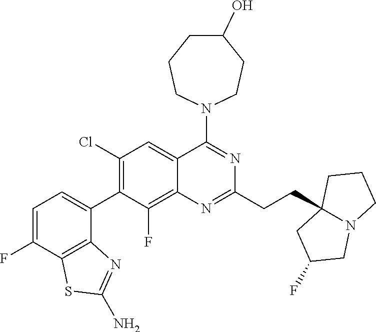 [SMILES: Nc1nc2c(-c3c(Cl)cc4c(N5CCCC(O)CC5)nc(CC[C@@]56CCCN5C[C@H](F)C6)nc4c3F)ccc(F)c2s1] | 1-(7-(2-amino-7-fluoro- benzo[d]thiazol-4-yl)-6- chloro-8-fluoro-2-(((2R,7aS)- 2-fluorotetrahydro-1H-pyr- rolizin-7a(5H)-yl)methoxy)- quinazolin-4-yl)azepan-4-ol | 619.1 |
| 408 | 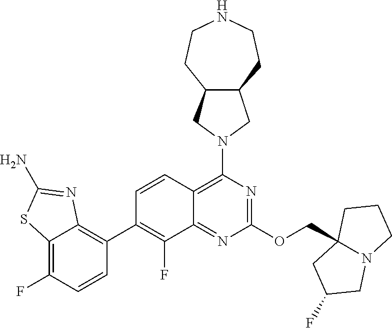 [SMILES: Nc1nc2c(-c3ccc4c(N5C[C@H]6CCNCC[C@H]6C5)nc(OC[C@@]56CCCN5C[C@H](F)C6)nc4c3F)ccc(F)c2s1] | 7-fluoro-4-(8-fluoro-2- (((2R,7aS)-2-fluorotetra- hydro-1H-pyrrolizin-7a(5H)- yl)methoxy)-4-((3aR,8aS)- octahydropyrrolo[3,4-d]- azepin-2(1H)-yl)quinazolin- 7-yl)benzo[d]thiazol-2- amine | 610.4 |
| 409 | 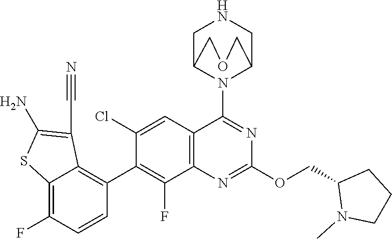 [SMILES: CN1CCC[C@H]1COc1nc(N2C3CNCC2COC3)c2cc(Cl)c(-c3ccc(F)c4sc(N)c(C#N)c34)c(F)c2n1] | 4-(4-(3-oxa-7,9-diaza- bicyclo[3.3.1]nonan-9-yl)- 6-chloro-8-fluoro-2-(((S)-1- methylpyrrolidin-2-yl)meth- oxy)quinazolin-7-yl)-2- amino-7-fluorobenzo[b]- thiophene-3-carbonitrile | 612.4 |
| 410 | 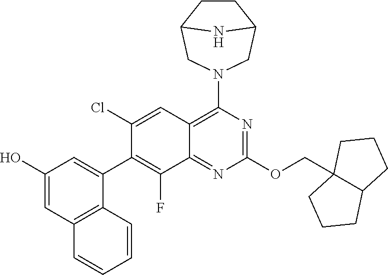 [SMILES: Oc1cc(-c2c(Cl)cc3c(N4CC5CCC(C4)N5)nc(OCC45CCCC4CCC5)nc3c2F)c2ccccc2c1] | 4-(4-(3,8-diazabicyclo- [3.2.1]octan-3-yl)-6-chloro- 8-fluoro-2-((hexahydro- pentalen-3a(1H)-yl)meth- oxy)quinazolin-7-yl)naph- thalen-2-ol | 573.1 |
| 411 | 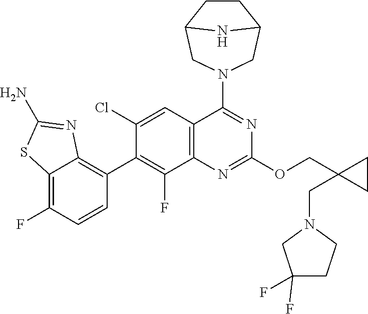 [SMILES: Nc1nc2c(-c3c(Cl)cc4c(N5CC6CCC(C5)N6)nc(OCC5(CN6CCC(F)(F)C6)CC5)nc4c3F)ccc(F)c2s1] | 4-(4-(3,8-diazabicyclo- [3.2.1]octan-3-yl)-6-chloro- 2-((1-((3,3-difluoropyrrol- idin-1-yl)methyl)cycloprop- yl)methoxy)-8-fluoroquin- azolin-7-yl)-7-fluorobenzo- [d]thiazol-2-amine | 646.3 [M − H]− |
| 412 | 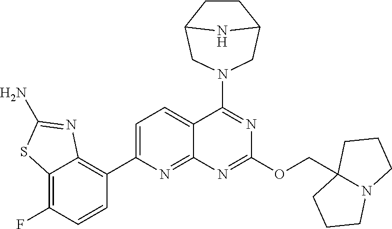 [SMILES: Nc1nc2c(-c3ccc4c(N5CC6CCC(C5)N6)nc(OCC56CCCN5CCC6)nc4n3)ccc(F)c2s1] | 4-(4-(3,8-diazabicyclo- [3.2.1]octan-3-yl)-2-((tetra- hydro-1H-pyrrolizin-7a(5H)- yl)methoxy)pyrido[2,3-d]- pyrimidin-7-yl)-7-fluoro- benzo[d]thiazol-2-amine | 547 |
| 413 | 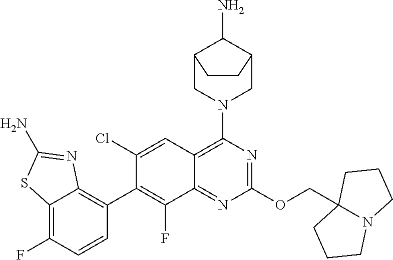 [SMILES: Nc1nc2c(-c3c(Cl)cc4c(N5CC6CCC(C5)C6N)nc(OCC56CCCN5CCC6)nc4c3F)ccc(F)c2s1] | 4-(4-((1R,5S,8s)-8-amino-3- azabicyclo[3.2.1]octan-3-yl)- 6-chloro-8-fluoro-2-((tetra- hydro-1H-pyrrolizin-7a(5H)- yl)methoxy)quinazolin-7-yl)- 7-fluorobenzo[d]thiazol-2- amine | 613.2 |
| 414 | 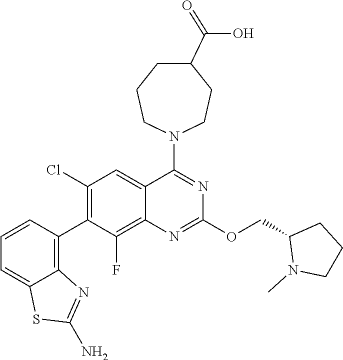 [SMILES: CN1CCC[C@H]1COc1nc(N2CCCC(C(=O)O)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)azepane- 4-carboxylic acid | 585.5 |
| 415 | 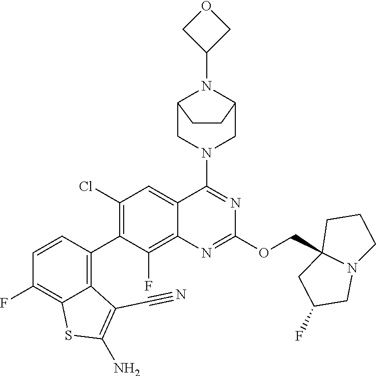 [SMILES: N#Cc1c(N)sc2c(F)ccc(-c3c(Cl)cc4c(N5CC6CCC(C5)N6C5COC5)nc(OC[C@@]56CCCN5C[C@H](F)C6)nc4c3F)c12] | 2-amino-4-(6-chloro-8- fluoro-2-(((2R,7aS)-2- fluorotetrahydro-1H- pyrrolizin-7a(5H)-yl)- methoxy)-4-((1R,5S)-8- (oxetan-3-yl)-3,8-diaza- bicyclo[3.2.1]octan-3-yl)- quinazolin-7-yl)-7-fluoro- benzo[b]thiophene-3- carbonitrile | 696.2 |
| 416 | 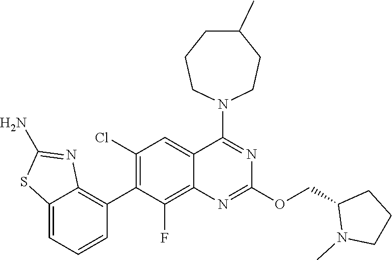 [SMILES: CC1CCCN(c2nc(OC[C@@H]3CCCN3C)nc3c(F)c(-c4cccc5sc(N)nc45)c(Cl)cc23)CC1] | 4-(6-chloro-8-fluoro-4-(4- methylazepan-1-yl)-2-(((S)- 1-methylpyrrolidin-2-yl)- methoxy)quinazolin-7-yl)- benzo[d]thiazol-2-amine | 555.1 |
| 417 | 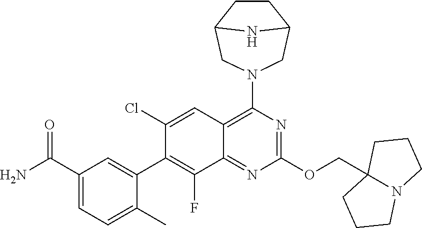 [SMILES: Cc1ccc(C(N)=O)cc1-c1c(Cl)cc2c(N3CC4CCC(C3)N4)nc(OCC34CCCN3CCC4)nc2c1F] | 3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-4- methylbenzamide | 565.2 |
| 418 | 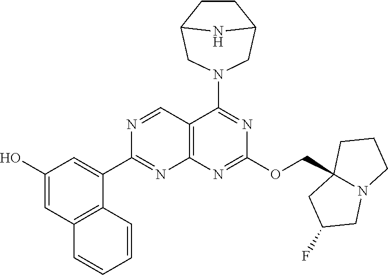 [SMILES: Oc1cc(-c2ncc3c(N4CC5CCC(C4)N5)nc(OC[C@@]45CCCN4C[C@H](F)C5)nc3n2)c2ccccc2c1] | 4-(5-(3,8-diazabicyclo- [3.2.1]octan-3-yl)-7- (((2R,7aS)-2-fluorotetra- hydro-1H-pyrrolizin- 7a(5H)-yl)methoxy)pyrim- ido[4,5-d]pyrimidin-2-yl)- naphthalen-2-ol | 542.5 |
| 419 | 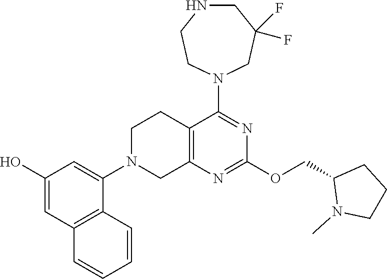 [SMILES: CN1CCC[C@H]1COc1nc2c(c(N3CCNCC(F)(F)C3)n1)CCN(c1cc(O)cc3ccccc13)C2] | (S)-4-(4-(6,6-difluoro-1,4- diazepan-1-yl)-2-((1-methyl- pyrrolidin-2-yl)methoxy)- 5,8-dihydropyrido[3,4-d]- pyrimidin-7(6H)-yl)naph- thalen-2-ol | 525.5 |
| 420 | 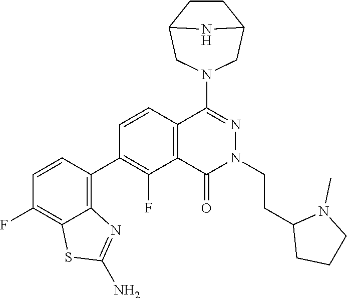 [SMILES: CN1CCCC1CCn1nc(N2CC3CCC(C2)N3)c2ccc(-c3ccc(F)c4sc(N)nc34)c(F)c2c1=O] | 7-(2-amino-7-fluorobenzo- [d]thiazol-4-yl)-4-(3,8- diazabicyclo[3.2.1]octan-3- yl)-8-fluoro-2-(2-(1-methyl- pyrrolidin-2-yl)ethyl)phthal- azin-1(2H)-one | 552.1 |
| 421 | 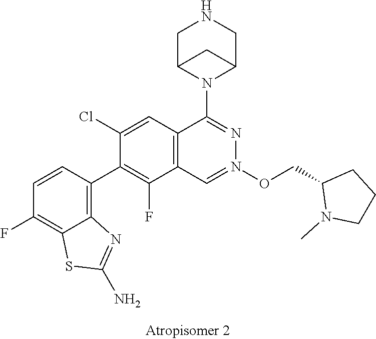 | 4-(4-(3,6-diazabicyclo- [3.1.1]heptan-6-yl)-6-chloro- 8-fluoro-2-(((S)-1-methyl- pyrrolidin-2-yl)methoxy)- quinazolin-7-yl)-7-fluoro- benzo[d]thiazol-2-amine | 558.1 |
| 422 | 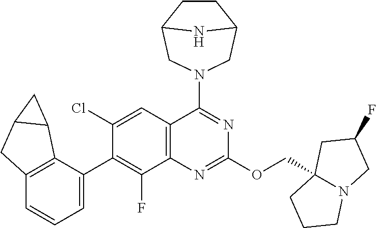 [SMILES: Fc1c(-c2cccc3c2C2CC2C3)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-6-chloro-8- fluoro-2-(((2R,7aS)-2-fluoro- tetrahydro-1H-pyrrolizin- 7a(5H)-yl)methoxy)-7- (1,1a,6,6a-tetrahydrocyclo- propa[a]inden-2-yl)quin- azoline | 578.2 |
| 423 | 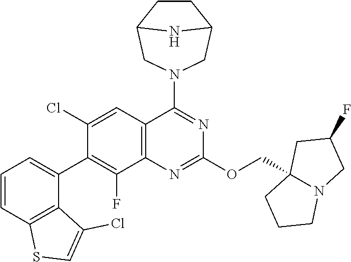 [SMILES: Fc1c(-c2cccc3scc(Cl)c23)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-6-chloro-7-(3- chlorobenzo[b]thiophen-4- yl)-8-fluoro-2-(((2R,7aS)-2- fluorotetrahydro-1H- pyrrolizin-7a(5H)-yl)meth- oxy)quinazoline | 616.2 |
| 424 | 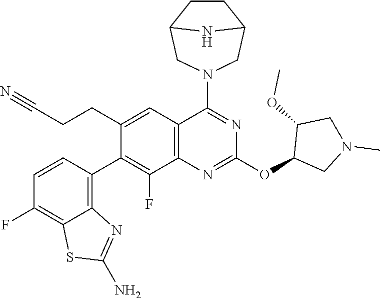 [SMILES: CO[C@@H]1CN(C)C[C@H]1Oc1nc(N2CC3CCC(C2)N3)c2cc(CCC#N)c(-c3ccc(F)c4sc(N)nc34)c(F)c2n1] | 3-(7-(2-amino-7-fluoro- benzo[d]thiazol-4-yl)-4-(3,8- diazabicyclo[3.2.1]octan-3- yl)-8-fluoro-2-(((3R,4R)-4- methoxy-1-methylpyrrolidin- 3-yl)oxy)quinazolin-6-yl)- propanenitrile | 605.4 [M − H]− |
| 425 | 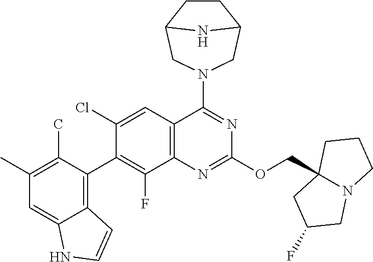 [SMILES: Cc1cc2[nH]ccc2c(-c2c(Cl)cc3c(N4CC5CCC(C4)N5)nc(OC[C@@]45CCCN4C[C@H](F)C5)nc3c2F)c1C] | 4-(3,8-diazabicyclo[3.2.1]- octan-3-yl)-6-chloro-7-(5- chloro-6-methyl-1H-indazol- 4-yl)-8-fluoro-2-(((2R,7aS)- 2-fluorotetrahydro-1H- pyrrolizin-7a(5H)-yl)meth- oxy)quinazoline | 614.2 |
| 426 | 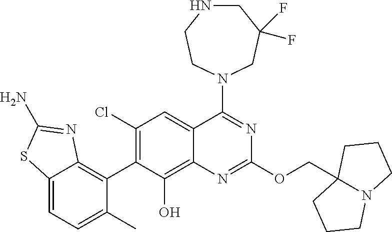 [SMILES: Cc1ccc2sc(N)nc2c1-c1c(Cl)cc2c(N3CCNCC(F)(F)C3)nc(OCC34CCCN3CCC4)nc2c1O] | 7-(2-amino-5-methylbenzo- [d]thiazol-4-yl)-6-chloro-4- (6,6-difluoro-1,4-diazepan- 1-yl)-2-((tetrahydro-1H- pyrrolizin-7a(5H)-yl)meth- oxy)quinazolin-8-ol | 616.3 |
| 427 | 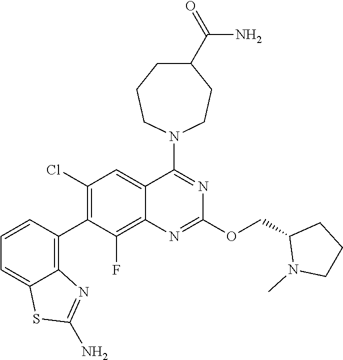 [SMILES: CN1CCC[C@H]1COc1nc(N2CCCC(C(N)=O)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methylpyr- rolidin-2-yl)methoxy)quin- azolin-4-yl)azepane-4- carboxamide | 584.3 |
| 428 | 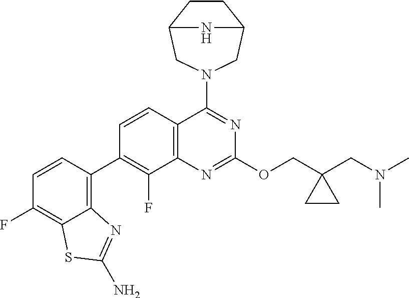 [SMILES: CN(C)CC1(COc2nc(N3CC4CCC(C3)N4)c3ccc(-c4ccc(F)c5sc(N)nc45)c(F)c3n2)CC1] | 4-(4-(3,8-diazabicyclo- [3.2.1]-octan-3-yl)-2-((1- ((dimethylamino)methyl)- cyclopropyl)methoxy)-8- fluoroquinazolin-7-yl)-7- fluorobenzo[d]thiazol-2- amine | 552.4 |
| 429 | 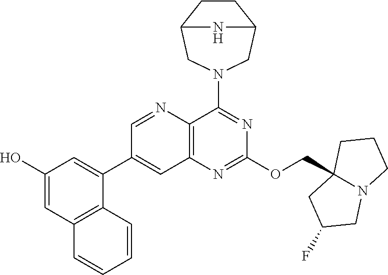 [SMILES: Oc1cc(-c2cnc3c(N4CC5CCC(C4)N5)nc(OC[C@@]45CCCN4C[C@H](F)C5)nc3c2)c2ccccc2c1] | 4-(4-(3,8-diazabicyclo- [3.2.1]octan-3-yl)-2- (((2R,7aS)-2-fluorotetra- hydro-1H-pyrrolizin- 7a(5H)-yl)methoxy)- pyrido[3,2-d]pyrimidin- 7-yl)naphthalen-2-ol | 541.5 |
| 430 | 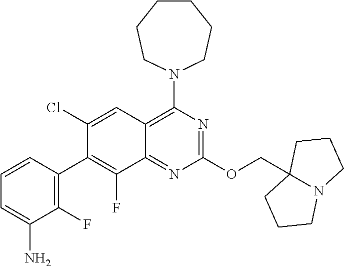 [SMILES: Nc1cccc(-c2c(Cl)cc3c(N4CCCCCC4)nc(OCC45CCCN4CCC5)nc3c2F)c1F] | 3-(4-(azepan-1-yl)-6-chloro- 8-fluoro-2-((tetrahydro-1H- pyrrolizin-7a(5H)-yl)meth- oxy)quinazolin-7-yl)-2- fluoroaniline | 528.2 |
| 431 | 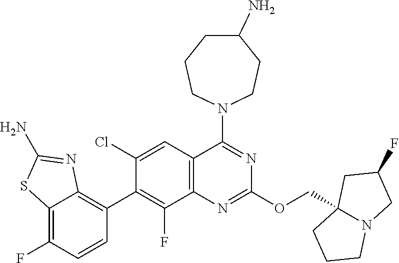 [SMILES: Nc1nc2c(-c3c(Cl)cc4c(N5CCCC(N)CC5)nc(OC[C@@]56CCCN5C[C@H](F)C6)nc4c3F)ccc(F)c2s1] | 4-(4-(4-aminoazepan-1-yl)- 6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 618.4 |
| 432 | 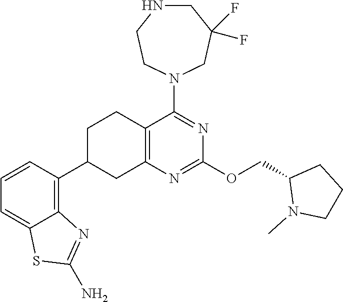 [SMILES: CN1CCC[C@H]1COc1nc2c(c(N3CCNCC(F)(F)C3)n1)CCC(c1cccc3sc(N)nc13)C2] | 4-(4-(6,6-difluoro-1,4- diazepan-1-yl)-2-(((S)-1- methylpyrrolidin-2-yl)meth- oxy)-5,6,7,8-tetrahydroquin- azolin-7-yl)benzo[d]thiazol- 2-amine | 530.2 |
| 433 | 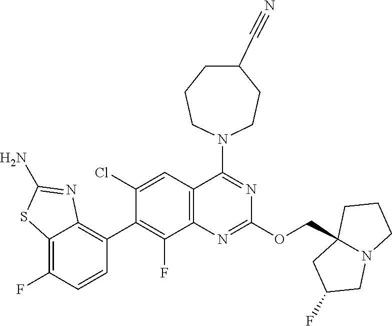 [SMILES: N#CC1CCCN(c2nc(OC[C@@]34CCCN3C[C@H](F)C4)nc3c(F)c(-c4ccc(F)c5sc(N)nc45)c(Cl)cc23)CC1] | 1-(7-(2-amino-7-fluoro- benzo[d]thiazol-4-yl)-6- chloro-8-fluoro-2- (((2R,7aS)-2-fluorotetra- hydro-1H-pyrrolizin- 7a(5H)-yl)methoxy)quin- azolin-4-yl)azepane-4- carbonitrile | 629.2 |
| 434 | 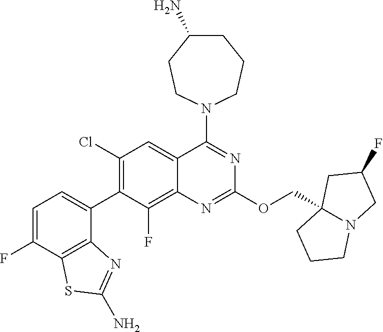 [SMILES: Nc1nc2c(-c3c(Cl)cc4c(N5CCC[C@@H](N)CC5)nc(OC[C@@]56CCCN5C[C@H](F)C6)nc4c3F)ccc(F)c2s1] | 4-(4-((R)-4-aminoazepan-1- yl)-6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 618.5 |
| 435 | 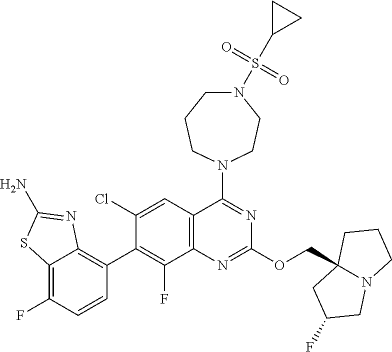 [SMILES: Nc1nc2c(-c3c(Cl)cc4c(N5CCCN(S(=O)(=O)C6CC6)CC5)nc(OC[C@@]56CCCN5C[C@H](F)C6)nc4c3F)ccc(F)c2s1] | 4-(6-chloro-4-(4-(cyclo- propylsulfonyl)-1,4- diazepan-1-yl)-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 708.5 |
| 436 | 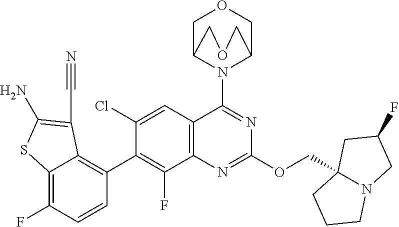 [SMILES: N#Cc1c(N)sc2c(F)ccc(-c3c(Cl)cc4c(N5C6COCC5COC6)nc(OC[C@@]56CCCN5C[C@H](F)C6)nc4c3F)c12] | 4-(4-(3,7-dioxa-9-azabi- cyclo[3.3.1]nonan-9-yl)-6- chloro-8-fluoro-2-(((2R,7aS)- 2-fluorotetrahydro-1H- pyrrolizin-7a(5H)-yl)meth- oxy)quinazolin-7-yl)-2- amino-7-fluorobenzo[b]- thiophene-3-carbonitrile | 657.4 |
| 437 | 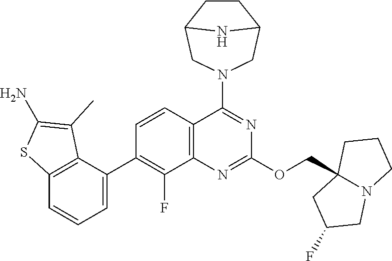 [SMILES: Cc1c(N)sc2cccc(-c3ccc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-3-methylbenzo[b]thio- phen-2-amine | 577.5 |
| 438 | 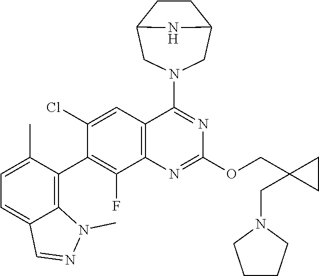 [SMILES: Cc1ccc2cnn(C)c2c1-c1c(Cl)cc2c(N3CC4CCC(C3)N4)nc(OCC3(CN4CCCC4)CC3)nc2c1F] | 4-(3,8-diazabicyclo[3.2.1]- octan-3-yl)-6-chloro-7-(1,6- dimethyl-1H-indazol-7-yl)- 8-fluoro-2-((1-(pyrrolidin-1- ylmethyl)cyclopropyl)meth- oxy)quinazoline | 590.5 |
| 439 | 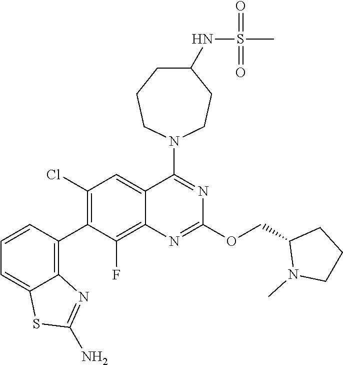 [SMILES: CN1CCC[C@H]1COc1nc(N2CCCC(NS(C)(=O)=O)CC2)c2cc(Cl)c(-c3cccc4sc(N)nc34)c(F)c2n1] | N-(1-(7-(2-aminobenzo[d]- thiazol-4-yl)-6-chloro-8- fluoro-2-(((S)-1-methyl- pyrrolidin-2-yl)methoxy)- quinazolin-4-yl)azepan-4- yl)methanesulfonamide | 634.3 |
| 440 | 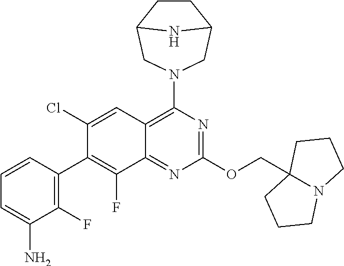 [SMILES: Nc1cccc(-c2c(Cl)cc3c(N4CC5CCC(C4)N5)nc(OCC45CCCN4CCC5)nc3c2F)c1F] | 3-(4-(3,8-diazabicyclo- [3.2.1]-octan-3-yl)-6-chloro- 8-fluoro-2-((tetrahydro-1H- pyrrolizin-7a(5H)-yl)meth- oxy)quinazolin-7-yl)-2- fluoroaniline | 542.2 |
| 441 | 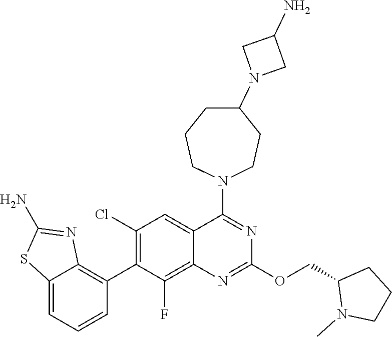 [SMILES: CN1CCC[C@H]1COc1nc(N2CCCC(N3CC(N)C3)CC2)c2cc(Cl)c(-c3cccc4sc(N)nc34)c(F)c2n1] | 4-(4-(4-(3-aminoazetidin-1- yl)azepan-1-yl)-6-chloro-8- fluoro-2-(((S)-1-methyl- pyrrolidin-2-yl)methoxy)- quinazolin-7-yl)benzo[d]- thiazol-2-amine | 611.3 |
| 442 | 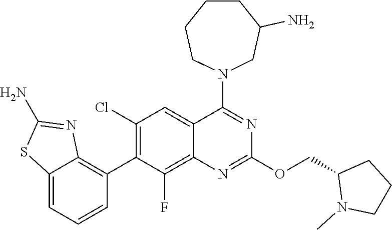 [SMILES: CN1CCC[C@H]1COc1nc(N2CCCCC(N)C2)c2cc(Cl)c(-c3cccc4sc(N)nc34)c(F)c2n1] | 4-(4-(3-aminoazepan-1-yl)- 6-chloro-8-fluoro-2-(((S)-1- methylpyrrolidin-2-yl)- methoxy)quinazolin-7-yl)- benzo[d]thiazol-2-amine | 556.2 |
| 443 | 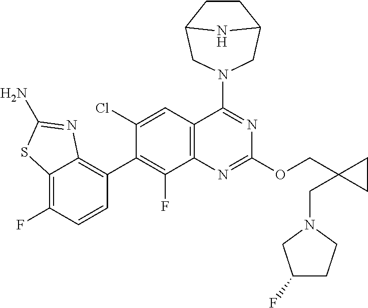 [SMILES: Nc1nc2c(-c3c(Cl)cc4c(N5CC6CCC(C5)N6)nc(OCC5(CN6CC[C@H](F)C6)CC5)nc4c3F)ccc(F)c2s1] | 4-(4-(3,8-diazabicyclo- [3.2.1]octan-3-yl)-6-chloro- 8-fluoro-2-((1-(((S)-3- fluoropyrrolidin-1-yl)meth- yl)cyclopropyl)methoxy)- quinazolin-7-yl)-7-fluoro- benzo[d]thiazol-2-amine | 630.4 |
| 444 | 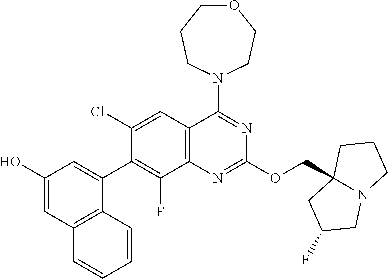 [SMILES: Oc1cc(-c2c(Cl)cc3c(N4CCCOCC4)nc(OC[C@@]45CCCN4C[C@H](F)C5)nc3c2F)c2ccccc2c1] | 4-(6-chloro-8-fluoro-2- (((2R,7aS)-2-fluorotetra- hydro-1H-pyrrolizin-7a(5H)- yl)methoxy)-4-(1,4-oxa- zepan-4-yl)quinazolin-7-yl)- naphthalen-2-ol | 581.3 |
| 445 | 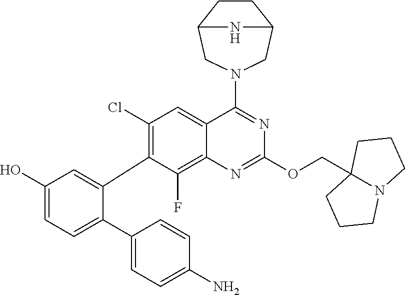 [SMILES: Nc1ccc(-c2ccc(O)cc2-c2c(Cl)cc3c(N4CC5CCC(C4)N5)nc(OCC45CCCN4CCC5)nc3c2F)cc1] | 2-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-4′- amino-[1,1′-biphenyl]-4-ol | 615.2 |
| 446 | 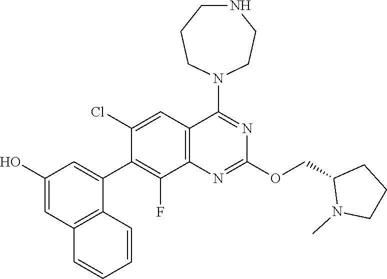 [SMILES: CN1CCC[C@H]1COc1nc(N2CCCNCC2)c2cc(Cl)c(-c3cc(O)cc4ccccc34)c(F)c2n1] | 4-(6-chloro-4-(1,4-diazepan- 1-yl)-8-fluoro-2-(((S)-1- methylpyrrolidin-2-yl)meth- oxy)quinazolin-7-yl)naph- thalen-2-ol | 536.3 |
| 447 | 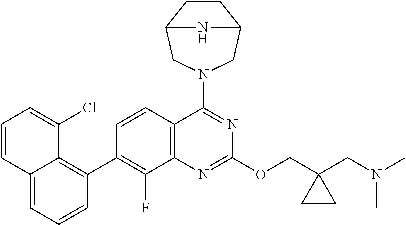 [SMILES: CN(C)CC1(COc2nc(N3CC4CCC(C3)N4)c3ccc(-c4cccc5cccc(Cl)c45)c(F)c3n2)CC1] | 1-(1-(((4-(3,8-diazabicyclo- [3.2.1]octan-3-yl)-7-(8- chloronaphthalen-1-yl)-8- fluoroquinazolin-2-yl)oxy)- methyl)cyclopropyl)-N,N- dimethylmethanamine | 546.5 |
| 448 | 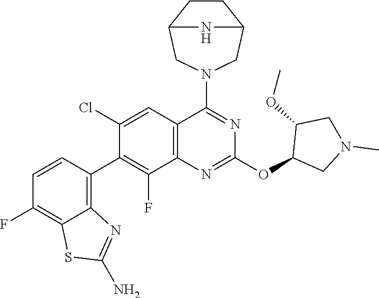 [SMILES: CO[C@@H]1CN(C)C[C@H]1Oc1nc(N2CC3CCC(C2)N3)c2cc(Cl)c(-c3ccc(F)c4sc(N)nc34)c(F)c2n1] | 4-(4-(3,8-diazabicyclo- [3.2.1]-octan-3-yl)-6-chloro- 8-fluoro-2-(((3R,4R)-4- methoxy-1-methylpyrrolidin- 3-yl)oxy)quinazolin-7-yl)- 7-fluorobenzo[d]thiazol-2- amine | 588.3 |
| 449 | 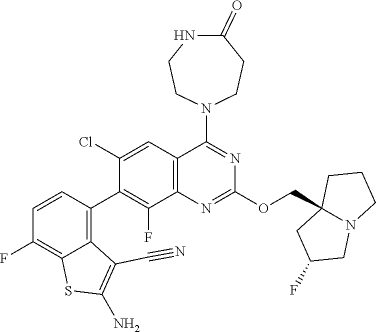 [SMILES: N#Cc1c(N)sc2c(F)ccc(-c3c(Cl)cc4c(N5CCNC(=O)CC5)nc(OC[C@@]56CCCN5C[C@H](F)C6)nc4c3F)c12] | 2-amino-4-(6-chloro-8- fluoro-2-(((2R,7aS)-2-fluoro- tetrahydro-1H-pyrrolizin- 7a(5H)-yl)methoxy)-4-(5- oxo-1,4-diazepan-1-yl)quin- azolin-7-yl)-7-fluorobenzo- [b]thiophene-3-carbonitrile | 642.2 |
| 450 | 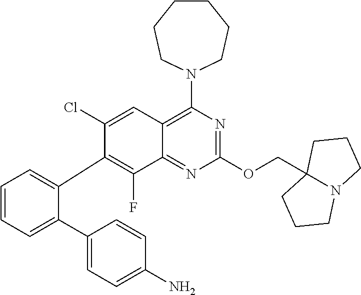 [SMILES: Nc1ccc(-c2ccccc2-c2c(Cl)cc3c(N4CCCCCC4)nc(OCC45CCCN4CCC5)nc3c2F)cc1] | 2′-(4-(azepan-1-yl)-6-chloro- 8-fluoro-2-((tetrahydro-1H- pyrrolizin-7a(5H)-yl)meth- oxy)quinazolin-7-yl)-[1,1′- biphenyl]-4-amine | 586.3 |
| 451 | 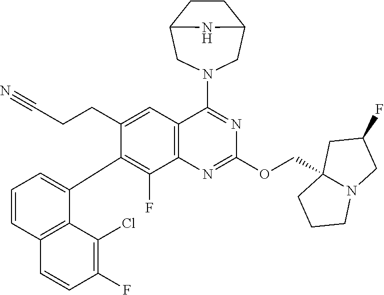 [SMILES: N#CCCc1cc2c(N3CC4CCC(C3)N4)nc(OC[C@@]34CCCN3C[C@H](F)C4)nc2c(F)c1-c1cccc2ccc(F)c(Cl)c12] | 3-(4-(3,8-diazabicyclo- [3.2.1]octan-3-yl)-7-(8- chloro-7-fluoronaphthalen- 1-yl)-8-fluoro-2-(((2R,7aS)- 2-fluorotetrahydro-1H- pyrrolizin-7a(5H)-yl)meth- oxy)quinazolin-6-yl)pro- panenitrile | 647.4 |
| 452 | 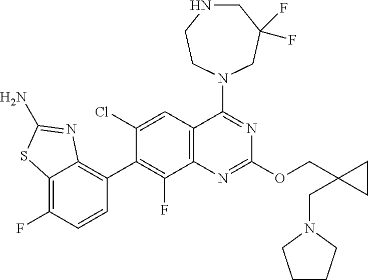 [SMILES: Nc1nc2c(-c3c(Cl)cc4c(N5CCNCC(F)(F)C5)nc(OCC5(CN6CCCC6)CC5)nc4c3F)ccc(F)c2s1] | 4-(6-chloro-4-(6,6-difluoro- 1,4-diazepan-1-yl)-8-fluoro- 2-((1-(pyrrolidin-1-ylmeth- yl)cyclopropyl)methoxy)- quinazolin-7-yl)-7-fluoro- benzo[d]thiazol-2-amine | 636.5 |
| 453 | 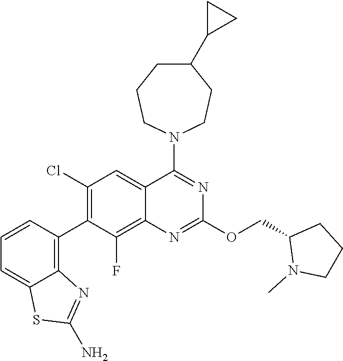 [SMILES: CN1CCC[C@H]1COc1nc(N2CCCC(C3CC3)CC2)c2cc(Cl)c(-c3cccc4sc(N)nc34)c(F)c2n1] | 4-(6-chloro-4-(4-cycloprop- propylazepan-1-yl)-8-fluoro- 2-(((S)-1-methylpyrrolidin- 2-yl)methoxy)quinazolin-7- yl)benzo[d]thiazol-2-amine | 581.2 |
| 454 | 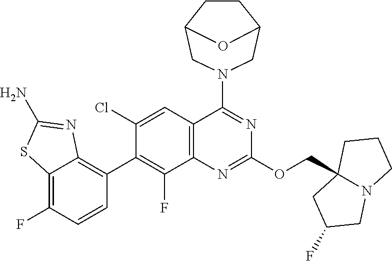 [SMILES: Nc1nc2c(-c3c(Cl)cc4c(N5CC6CCC(C5)O6)nc(OC[C@@]56CCCN5C[C@H](F)C6)nc4c3F)ccc(F)c2s1] | 4-(4-(8-oxa-3-azabicyclo- [3.2.1]octan-3-yl)-6-chloro- 8-fluoro-2-(((2R,7aS)-2- fluorotetrahydro-1H- pyrrolizin-7a(5H)-yl)meth- oxy)quinazolin-7-yl)-7- fluorobenzo[d]thiazol-2- amine | 617.4 |
| 455 | 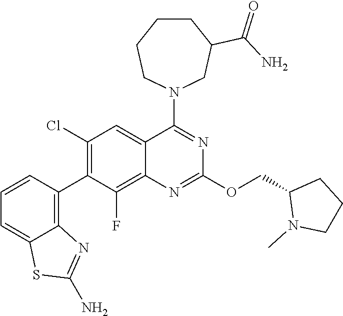 [SMILES: CN1CCC[C@H]1COc1nc(N2CCCCC(C(N)=O)C2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methylpyr- rolidin-2-yl)methoxy)quin- zolin-4-yl)azepane-3- carboxamide | 584.2 |
| 456 | 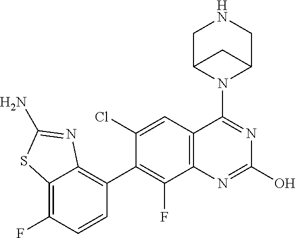 [SMILES: Nc1nc2c(-c3c(Cl)cc4c(N5C6CNCC5C6)nc(O)nc4c3F)ccc(F)c2s1] | 7-(2-amino-7-fluorobenzo- [d]thiazol-4-yl)-4-(3,6- diazabicyclo[3.1.1]heptan- 6-yl)-6-chloro-8-fluoroquin- azolin-2-ol | 461.0 |
| 457 | 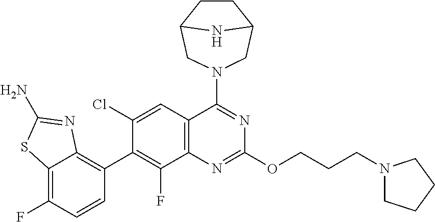 [SMILES: Nc1nc2c(-c3c(Cl)cc4c(N5CC6CCC(C5)N6)nc(OCCCN5CCCC5)nc4c3F)ccc(F)c2s1] | 4-(4-(3,8-diazabicyclo- [3.2.1]octan-3-yl)-6-chloro- 8-fluoro-2-(3-(pyrrolidin-1- yl)propoxy)quinazolin-7-yl)- 7-fluorobenzo[d]thiazol-2- amine | 586.5 |
| 458 | 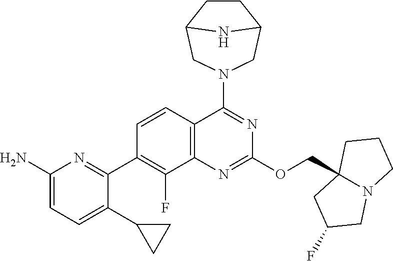 [SMILES: Nc1ccc(C2CC2)c(-c2ccc3c(N4CC5CCC(C4)N5)nc(OC[C@@]45CCCN4C[C@H](F)C5)nc3c2F)n1] | 6-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-5-cyclopropylpyridin-2- amine | 548.3 |
| 459 | 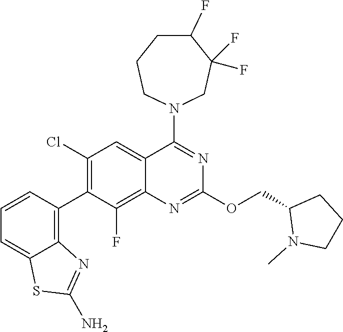 [SMILES: CN1CCC[C@H]1COc1nc(N2CCCC(F)C(F)(F)C2)c2cc(Cl)c(-c3cccc4sc(N)nc34)c(F)c2n1] | 4-(6-chloro-8-fluoro-2-(((S)- 1-methylpyrrolidin-2-yl)- methoxy)-4-(3,3,4-trifluoro- azepan-1-yl)quinazolin-7- yl)benzo[d]thiazol-2-amine | 595.3 |
| 460 | 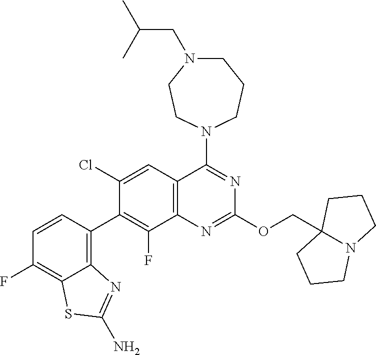 [SMILES: CC(C)CN1CCCN(c2nc(OCC34CCCN3CCC4)nc3c(F)c(-c4ccc(F)c5sc(N)nc45)c(Cl)cc23)CC1] | 4-(6-chloro-8-fluoro-4-(4- isobutyl-1,4-diazepan-1-yl)- 2-((tetrahydro-1H-pyrrolizin- 7a(5H)-yl)methoxy)quin- azolin-7-yl)-7-fluorobenzo- [d]thiazol-2-amine- | 642.4 |
| 461 | 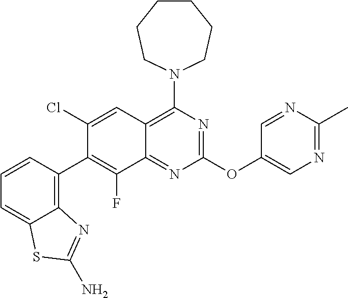 [SMILES: Cc1ncc(Oc2nc(N3CCCCCC3)c3cc(Cl)c(-c4cccc5sc(N)nc45)c(F)c3n2)cn1] | 4-(4-(azepan-1-yl)-6-chloro- 8-fluoro-2-((2-methylpyrim- idin-5-yl)oxy)quinazolin-7- yl)benzo[d]thiazol-2-amine | 537 |
| 462 | 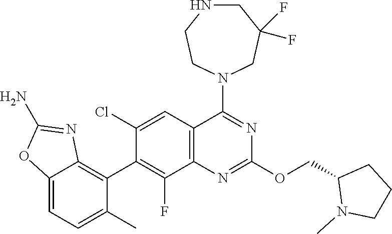 [SMILES: Cc1ccc2oc(N)nc2c1-c1c(Cl)cc2c(N3CCNCC(F)(F)C3)nc(OC[C@@H]3CCCN3C)nc2c1F] | 4-(6-chloro-4-(6,6-difluoro- 1,4-diazepan-1-yl)-8-fluoro- 2-(((S)-1-methylpyrrolidin- 2-yl)methoxy)quinazolin-7- yl)-5-methylbenzo[d]oxazol- 2-amine | 576.5 |
| 463 | 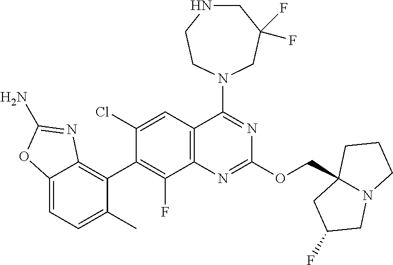 [SMILES: Cc1ccc2oc(N)nc2c1-c1c(Cl)cc2c(N3CCNCC(F)(F)C3)nc(OC[C@@]34CCCN3C[C@H](F)C4)nc2c1F] | 4-(6-chloro-4-(6,6-difluoro- 1,4-diazepan-1-yl)-8-fluoro- 2-(((2R,7aS)-2-fluorotetra- hydro-1H-pyrrolizin-7a(5H)- yl)methoxy)quinazolin-7-yl)- 5-methylbenzo[d]oxazol-2- amine | 620.5 |
| 464 | 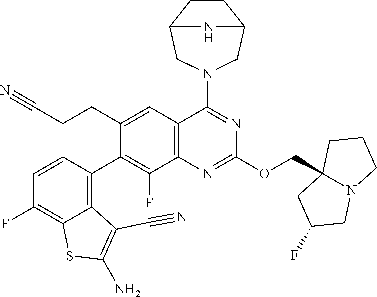 [SMILES: N#CCCc1cc2c(N3CC4CCC(C3)N4)nc(OC[C@@]34CCCN3C[C@H](F)C4)nc2c(F)c1-c1ccc(F)c2sc(N)c(C#N)c12] | 4-(4-(3,8-diazabicyclo- [3.2.1]octan-3-yl)-6-(2- cyanoethyl)-8-fluoro-2- (((2R,7aS)-2-fluorotetra- hydro-1H-pyrrolizin- 7a(5H)-yl)methoxy)quin- azolin-7-yl)-2-amino-7- fluorobenzo[b]thiophene- 3-carbonitrile | 659.4 |
| 465 | 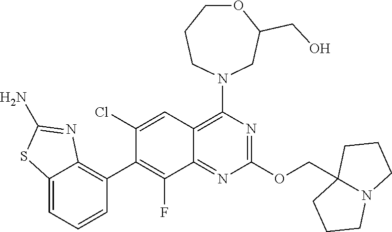 [SMILES: Nc1nc2c(-c3c(Cl)cc4c(N5CCCOC(CO)C5)nc(OCC56CCCN5CCC6)nc4c3F)cccc2s1] | (4-(7-(2-aminobenzo[d]- thiazol-4-yl)-6-chloro-8- fluoro-2-((tetrahydro-1H- pyrrolizin-7a(5H)-yl)meth- oxy)quinazolin-4-yl)-1,4- oxazepan-2-yl)methanol | 599.2 |
| 466 | 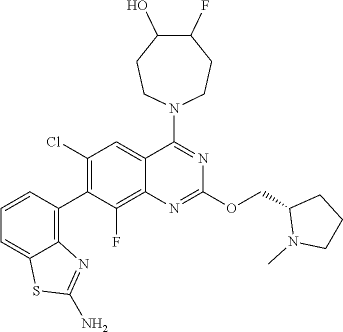 [SMILES: CN1CCC[C@H]1COc1nc(N2CCC(O)C(F)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methylpyr- rolidin-2-yl)methoxy)quin- azolin-4-yl)-5-fluoroazepan- 4-ol | 575.3 |
| 467 | 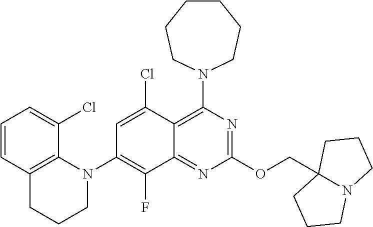 [SMILES: Fc1c(N2CCCc3cccc(Cl)c32)cc(Cl)c2c(N3CCCCCC3)nc(OCC34CCCN3CCC4)nc12] | 4-(azepan-1-yl)-5-chloro-7- (8-chloro-3,4-dihydroquin- olin-1(2H)-yl)-8-fluoro-2- ((tetrahydro-1H-pyrrolizin- 7a(5H)-yl)methoxy)quin- azoline | 585.2 |
| 468 | 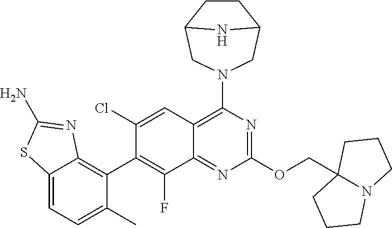 [SMILES: Cc1ccc2sc(N)nc2c1-c1c(Cl)cc2c(N3CC4CCC(C3)N4)nc(OCC34CCCN3CCC4)nc2c1F] | 4-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-5- methylbenzo[d]thiazol-2- amine | 594.2 |
| 469 | 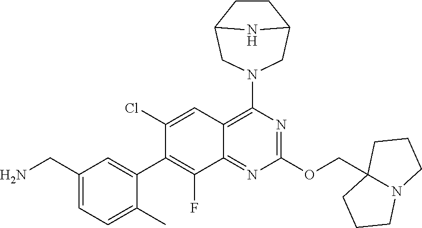 [SMILES: Cc1ccc(CN)cc1-c1c(Cl)cc2c(N3CC4CCC(C3)N4)nc(OCC34CCCN3CCC4)nc2c1F] | (3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-4- methylphenyl)methanamine | 551.2 |
| 470 | 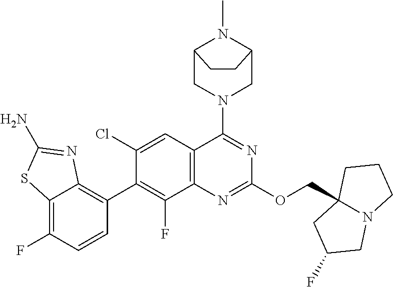 [SMILES: CN1C2CCC1CN(c1nc(OC[C@@]34CCCN3C[C@H](F)C4)nc3c(F)c(-c4ccc(F)c5sc(N)nc45)c(Cl)cc13)C2] | 4-(6-chloro-8-fluoro-2- (((2R,7aS)-2-fluorotetra- hydro-1H-pyrrolizin-7a(5H)- yl)methoxy)-4-((1R,5S)-8- methyl-3,8-diazabicyclo- [3.2.1]octan-3-yl)quinazolin- 7-yl)-7-fluorobenzo[d]- thiazol-2-amine | 630.5 |
| 471 | 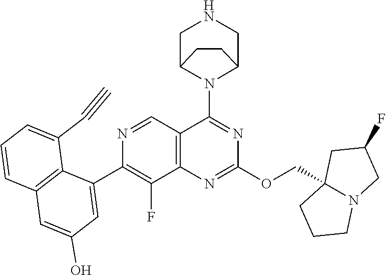 [SMILES: C#Cc1cccc2cc(O)cc(-c3ncc4c(N5C6CCC5CNC6)nc(OC[C@@]56CCCN5C[C@H](F)C6)nc4c3F)c12] | 4-(4-((1R,5S)-3,8-diaza- bicyclo-[3.2.1]octan-8-yl)- 8-fluoro-2-(((2R,7aS)-2- fluorotetrahydro-1H-pyrrol- izin-7a(5H)-yl)methoxy)- pyrido[4,3-d]pyrimidin-7- yl)-5-ethynylnaphthalen-2- ol | 583.5 |
| 472 | 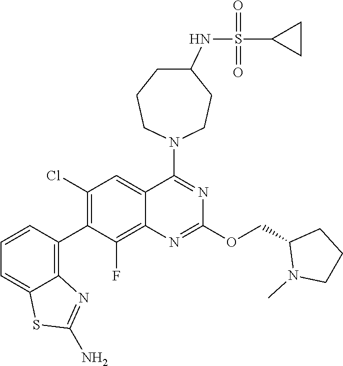 [SMILES: CN1CCC[C@H]1COc1nc(N2CCCC(NS(=O)(=O)C3CC3)CC2)c2cc(Cl)c(-c3cccc4sc(N)nc34)c(F)c2n1] | N-(1-(7-(2-aminobenzo[d]- thiazol-4-yl)-6-chloro-8- fluoro-2-(((S)-1-methylpyr- rolidin-2-yl)methoxy)quin- azolin-4-yl)azepan-4-yl)- cyclopropanesulfonamide | 660.3 |
| 473 | 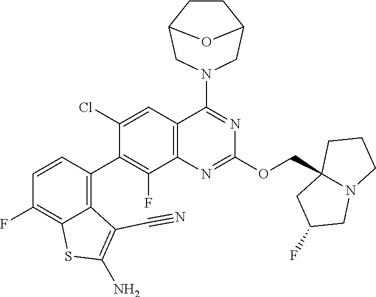 [SMILES: N#Cc1c(N)sc2c(F)ccc(-c3c(Cl)cc4c(N5CC6CCC(C5)O6)nc(OC[C@@]56CCCN5C[C@H](F)C6)nc4c3F)c12] | 4-(4-(8-oxa-3-azabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-2-amino-7- fluorobenzo[b]thiophene-3- carbonitrile | 641.2 |
| 474 | 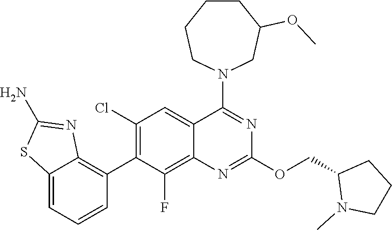 [SMILES: COC1CCCCN(c2nc(OC[C@@H]3CCCN3C)nc3c(F)c(-c4cccc5sc(N)nc45)c(Cl)cc23)C1] | 4-(6-chloro-8-fluoro-4-(3- methoxyazepan-1-yl)-2- (((S)-1-methylpyrrolidin-2- yl)methoxy)quinazolin-7- yl)benzo[d]thiazol-2-amine | 571.3 |
| 475 | 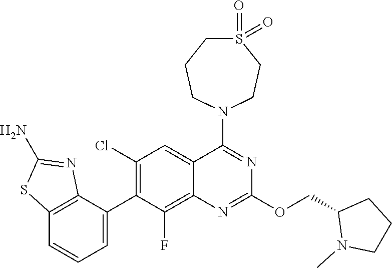 [SMILES: CN1CCC[C@H]1COc1nc(N2CCCS(=O)(=O)CC2)c2cc(Cl)c(-c3cccc4sc(N)nc34)c(F)c2n1] | 4-(7-(2-aminobenzo[d]- thiazol-4-yl)-6-chloro-8- fluoro-2-(((S)-1-methyl- pyrrolidin-2-yl)methoxy)- quinazolin-4-yl)-1,4- thiazepane 1,1-dioxide | 591.1 |
| 476 | 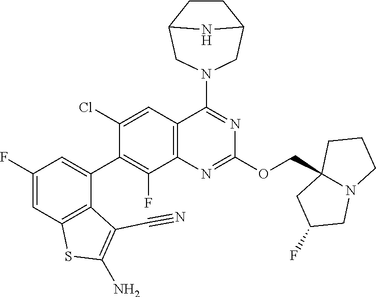 [SMILES: N#Cc1c(N)sc2cc(F)cc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-6-chloro- 8-fluoro-2-(((2R,7aS)-2- fluorotetrahydro-1H- pyrrolizin-7a(5H)-yl)meth- oxy)quinazolin-7-yl)-2- amino-6-fluorobenzo[b]- thiophene-3-carbonitrile | 640.2 |
| 477 | 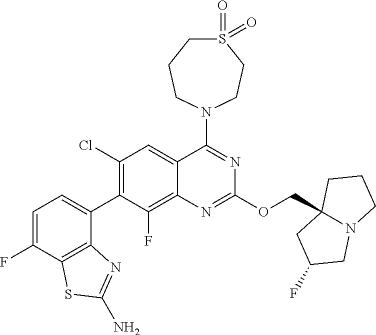 [SMILES: Nc1nc2c(-c3c(Cl)cc4c(N5CCCS(=O)(=O)CC5)nc(OC[C@@]56CCCN5C[C@H](F)C6)nc4c3F)ccc(F)c2s1] | 4-(7-(2-amino-7-fluoroben- zo-[d]thiazol-4-yl)-6-chloro- 8-fluoro-2-(((2R,7aS)-2- fluorotetrahydro-1H- pyrrolizin-7a(5H)-yl)meth- oxy)quinazolin-4-yl)-1,4- thiazepane 1,1-dioxide | 655.2 |
| 478 | 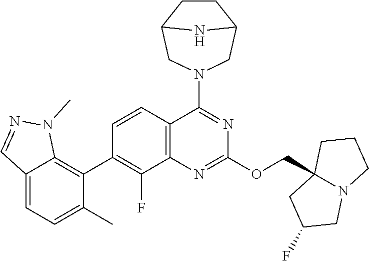 [SMILES: Cc1ccc2cnn(C)c2c1-c1ccc2c(N3CC4CCC(C3)N4)nc(OC[C@@]34CCCN3C[C@H](F)C4)nc2c1F] | 4-(3,8-diazabicyclo- [3.2.1]octan-3-yl)-7-(1,6- dimethyl-1H-indazol-7-yl)- 8-fluoro-2-(((2R,7aS)-2- fluorotetrahydro-1H- pyrrolizin-7a(5H)-yl)meth- oxy)quinazoline | 560.5 |
| 479 | 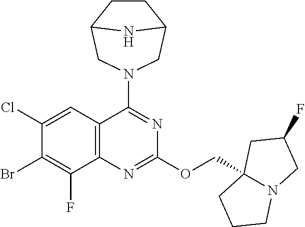 [SMILES: Fc1c(Br)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-7-bromo-6- chloro-8-fluoro-2- (((2R,7aS)-2-fluorotetra- hydro-1H-pyrrolizin- 7a(5H)-yl)methoxy)quin- azoline | 530.1 |
| 480 | 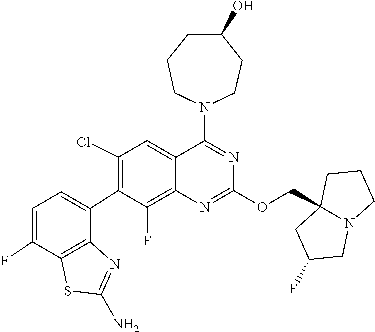 [SMILES: Nc1nc2c(-c3c(Cl)cc4c(N5CCC[C@@H](O)CC5)nc(OC[C@@]56CCCN5C[C@H](F)C6)nc4c3F)ccc(F)c2s1] | (4R)-1-(7-(2-amino-7- fluorobenzo[d]thiazol-4- yl)-6-chloro-8-fluoro-2- (((2R,7aS)-2-fluorotetra- hydro-1H-pyrrolizin-7a(5H)- yl)methoxy)quinazolin-4- yl)azepan-4-ol | 619.2 |
| 481 | 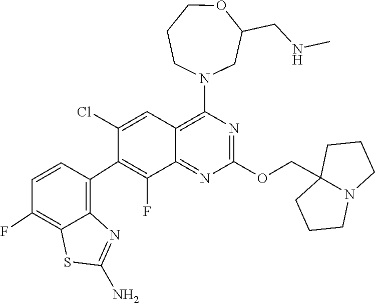 [SMILES: CNCC1CN(c2nc(OCC34CCCN3CCC4)nc3c(F)c(-c4ccc(F)c5sc(N)nc45)c(Cl)cc23)CCCO1] | 4-(6-chloro-8-fluoro-4-(2- ((methylamino)methyl)-1,4- oxazepan-4-yl)-2-((tetra- hydro-1H-pyrrolizin-7a(5H)- yl)methoxy)quinazolin-7- yl)-7-fluorobenzo[d]thiazol- 2-amine | 630.3 |
| 482 | 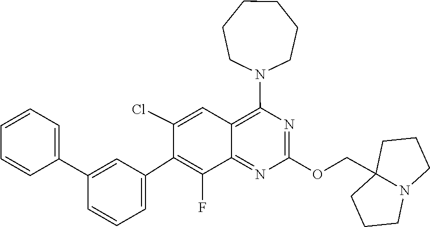 [SMILES: Fc1c(-c2cccc(-c3ccccc3)c2)c(Cl)cc2c(N3CCCCCC3)nc(OCC34CCCN3CCC4)nc12] | 7-([1,1′-biphenyl]-3-yl)-4- (azepan-1-yl)-6-chloro-8- fluoro-2-((tetrahydro-1H- pyrrolizin-7a(5H)-yl)meth- oxy)quinazoline | 571.2 |
| 483 | 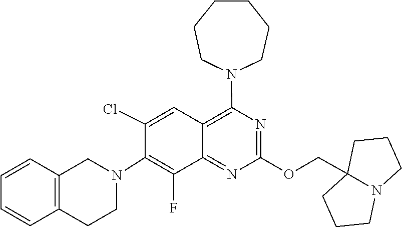 [SMILES: Fc1c(N2CCc3ccccc3C2)c(Cl)cc2c(N3CCCCCC3)nc(OCC34CCCN3CCC4)nc12] | 4-(azepan-1-yl)-7-(3,4- dihydroisoquinolin-2(1H)- yl)-8-fluoro-2-((tetrahydro- 1H-pyrrolizin-7a(5H)-yl)- methoxy)quinazoline | 515.2 |
| 484 | 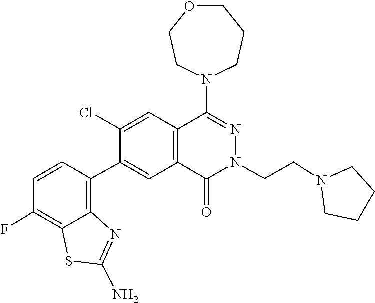 [SMILES: Nc1nc2c(-c3cc4c(=O)n(CCN5CCCC5)nc(N5CCCOCC5)c4cc3Cl)ccc(F)c2s1] | 7-(2-amino-7-fluorobenzo- [d]thiazol-4-yl)-6-chloro-4- (1,4-oxazepan-4-yl)-2-(2- (pyrrolidin-1-yl)ethyl)phthal- azin-1(2H)-one | 544.2 |
| 485 | 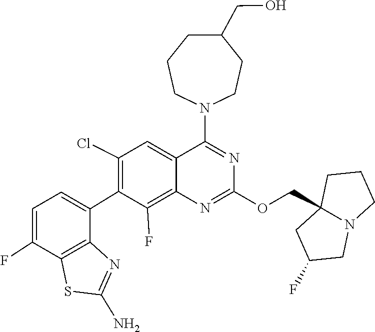 [SMILES: Nc1nc2c(-c3c(Cl)cc4c(N5CCCC(CO)CC5)nc(OC[C@@]56CCCN5C[C@H](F)C6)nc4c3F)ccc(F)c2s1] | (1-(7-(2-amino-7-fluoro- benzo[d]thiazol-4-yl)-6- chloro-8-fluoro-2- (((2R,7aS)-2-fluorotetra- hydro-1H-pyrrolizin-7a(5H)- yl)methoxy)quinazolin-4- yl)azepan-4-yl)methanol | 633.1 |
| 486 | 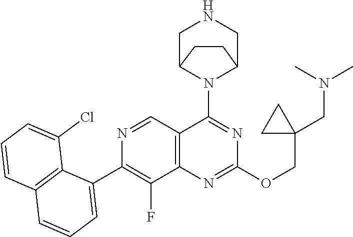 [SMILES: CN(C)CC1(COc2nc(N3C4CCC3CNC4)c3cnc(-c4cccc5cccc(Cl)c45)c(F)c3n2)CC1] | 1-(1-(((4-((1R,5S)-3,8- diazabicyclo[3.2.1]octan-8- yl)-7-(8-chloronaphthalen- 1-yl)-8-fluoropyrido[4,3- d]pyrimidin-2-yl)oxy)meth- yl)cyclopropyl)-N,N- dimethylmethanamine | 547.4 |
| 487 | 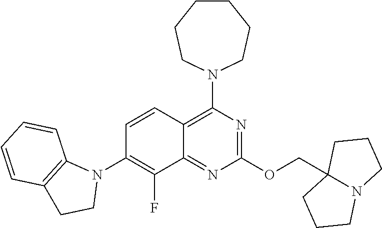 [SMILES: Fc1c(N2CCc3ccccc32)ccc2c(N3CCCCCC3)nc(OCC34CCCN3CCC4)nc12] | 4-(azepan-1-yl)-8-fluoro-7- (indolin-1-yl)-2-((tetra- hydro-1H-pyrrolizin- 7a(5H)-yl)methoxy)quin- azoline | 502.3 |
| 488 | 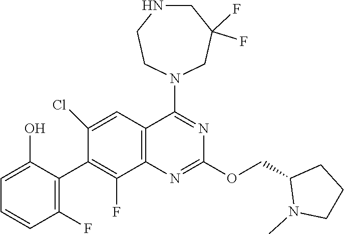 [SMILES: CN1CCC[C@H]1COc1nc(N2CCNCC(F)(F)C2)c2cc(Cl)c(-c3c(O)cccc3F)c(F)c2n1] | 2-(6-chloro-4-(6,6-difluoro- 1,4-diazepan-1-yl)-8-fluoro- 2-(((S)-1-methylpyrrolidin- 2-yl)methoxy)quinazolin-7- yl)-3-fluorophenol | 540.4 |
| 489 | 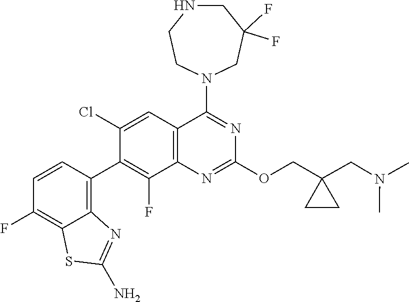 [SMILES: CN(C)CC1(COc2nc(N3CCNCC(F)(F)C3)c3cc(Cl)c(-c4ccc(F)c5sc(N)nc45)c(F)c3n2)CC1] | 4-(6-chloro-4-(6,6-difluoro- 1,4-diazepan-1-yl)-2-((1- ((dimethylamino)methyl)- cyclopropyl)methoxy)-8- fluoroquinazolin-7-yl)-7- fluorobenzo[d]thiazol-2- amine | 610.5 |
| 490 | 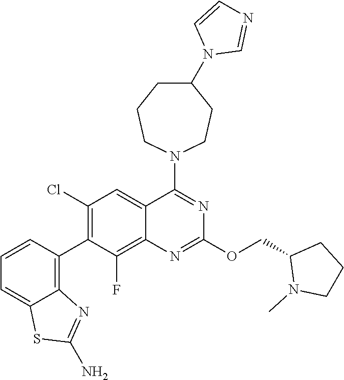 [SMILES: CN1CCC[C@H]1COc1nc(N2CCCC(n3ccnc3)CC2)c2cc(Cl)c(-c3cccc4sc(N)nc34)c(F)c2n1] | 4-(4-(4-(1H-imidazol-1-yl)- azepan-1-yl)-6-chloro-8- fluoro-2-(((S)-1-methylpyr- rolidin-2-yl)methoxy)quin- azolin-7-yl)benzo[d]thiazol- 2-amine | 607.3 |
| 491 | 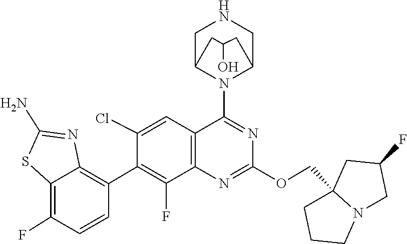 [SMILES: Nc1nc2c(-c3c(Cl)cc4c(N5C6CNCC5CC(O)C6)nc(OC[C@@]56CCCN5C[C@H](F)C6)nc4c3F)ccc(F)c2s1] | 9-(7-(2-amino-7-fluoro- benzo[d]thiazol-4-yl)-6- chloro-8-fluoro-2- (((2R,7aS)-2-fluorotetra- hydro-1H-pyrrolizin-7a(5H)- yl)methoxy)quinazolin-4- yl)-3,9-diazabicyclo[3.3.1]- nonan-7-ol | 647.3 |
| 492 | 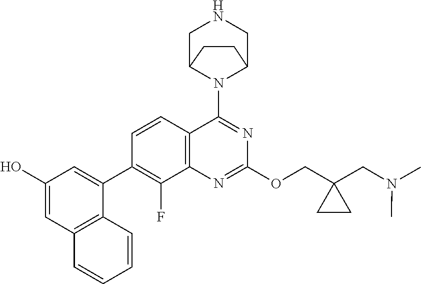 [SMILES: CN(C)CC1(COc2nc(N3C4CCC3CNC4)c3ccc(-c4cc(O)cc5ccccc45)c(F)c3n2)CC1] | 4-(4-((1R,5S)-3,8-diaza- bicyclo-[3.2.1]octan-8-yl)- 2-((1-((dimethylamino)- methyl)cyclopropyl)meth- oxy)-8-fluoroquinazolin-7- yl)naphthalen-2-ol | 528.5 |
| 493 | 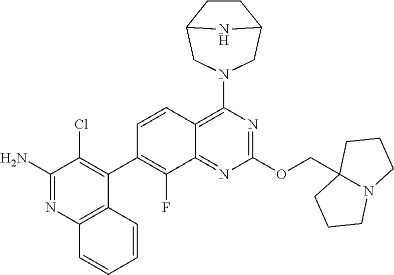 [SMILES: Nc1nc2ccccc2c(-c2ccc3c(N4CC5CCC(C4)N5)nc(OCC45CCCN4CCC5)nc3c2F)c1Cl] | 4-(4-(3,8-diazabicyclo- [3.2.1]octan-3-yl)-8-fluoro- 2-((tetrahydro-1H-pyrrol- izin-7a(5H)-yl)methoxy)- quinazolin-7-yl)-3-chloro- quinolin-2-amine | 575.3 |
| 494 | 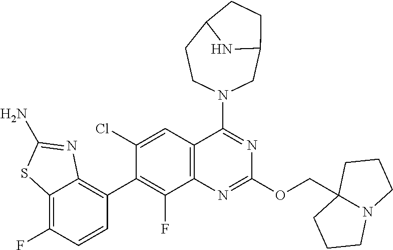 [SMILES: Nc1nc2c(-c3c(Cl)cc4c(N5CCC6CCC(C5)N6)nc(OCC56CCCN5CCC6)nc4c3F)ccc(F)c2s1] | 4-(4-(3,9-diazabicyclo- [4.2.1]nonan-3-yl)-6-chloro- 8-fluoro-2-((tetrahydro-1H- pyrrolizin-7a(5H)-yl)meth- oxy)quinazolin-7-yl)-7- fluorobenzo[d]thiazol-2- amine | 612.2 |
| 495 | 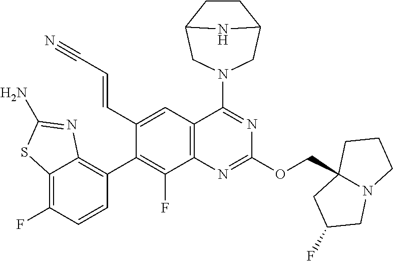 [SMILES: N#C/C=C/c1cc2c(N3CC4CCC(C3)N4)nc(OC[C@@]34CCCN3C[C@H](F)C4)nc2c(F)c1-c1ccc(F)c2sc(N)nc12] | (E)-3-(7-(2-amino-7-fluoro- benzo[d]thiazol-4-yl)-4-(3,8- diazabicyclo[3.2.1]octan-3- yl)-8-fluoro-2-(((2R,7aS)-2- fluorotetrahydro-1H-pyrrol- izin-7a(5H)-yl)methoxy)- quinazolin-6-yl)acrylonitrile | 633.4 |
| 496 | 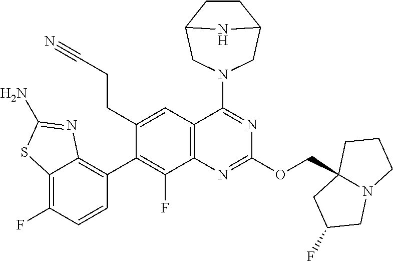 [SMILES: N#CCCc1cc2c(N3CC4CCC(C3)N4)nc(OC[C@@]34CCCN3C[C@H](F)C4)nc2c(F)c1-c1ccc(F)c2sc(N)nc12] | 3-(7-(2-amino-7-fluoroben- zo[d]thiazol-4-yl)-4-(3,8- diazabicyclo[3.2.1]octan-3- yl)-8-fluoro-2-(((2R,7aS)-2- fluorotetrahydro-1H-pyrrol- izin-7a(5H)-yl)methoxy)- quinazolin-6-yl)propane- nitrile | 635.4 |
| 497 | 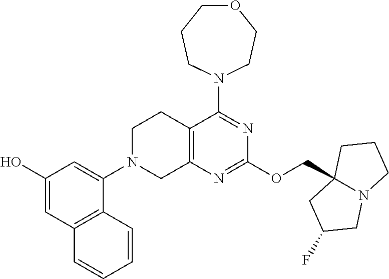 [SMILES: Oc1cc(N2CCc3c(nc(OC[C@@]45CCCN4C[C@H](F)C5)nc3N3CCCOCC3)C2)c2ccccc2c1] | 4-(2-(((2R,7aS)-2-fluoro- tetrahydro-1H-pyrrolizin- 7a(5H)-yl)methoxy)-4-(1,4- oxazepan-4-yl)-5,8-dihydro- pyrido[3,4-d]pyrimidin- 7(6H)-yl)naphthalen-2-ol | 534.4 |
| 498 | 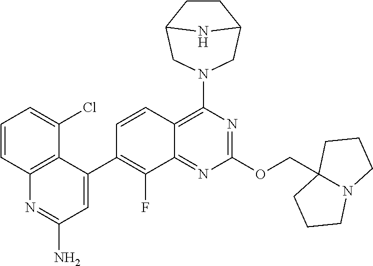 [SMILES: Nc1cc(-c2ccc3c(N4CC5CCC(C4)N5)nc(OCC45CCCN4CCC5)nc3c2F)c2c(Cl)cccc2n1] | 4-(4-(3,8-diazabicyclo- [3.2.1]octan-3-yl)-8-fluoro- 2-((tetrahydro-1H-pyrrol- izin-7a(5H)-yl)methoxy)- quinazolin-7-yl)-5-chloro- quinolin-2-amine | 574.2 |
| 499 | 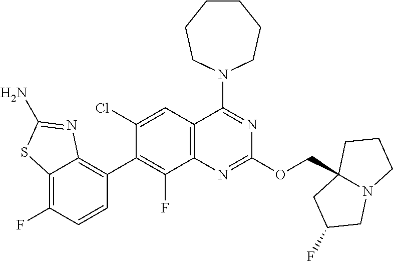 [SMILES: Nc1nc2c(-c3c(Cl)cc4c(N5CCCCCC5)nc(OC[C@@]56CCCN5C[C@H](F)C6)nc4c3F)ccc(F)c2s1] | 4-(4-(azepan-1-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 604.1 |
| 500 | 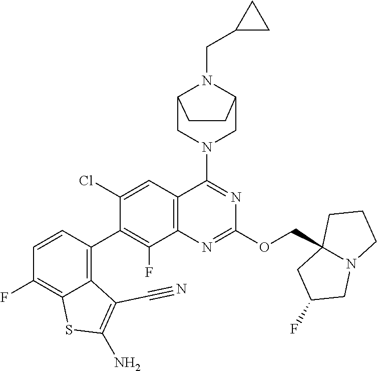 [SMILES: N#Cc1c(N)sc2c(F)ccc(-c3c(Cl)cc4c(N5CC6CCC(C5)N6CC5CC5)nc(OC[C@@]56CCCN5C[C@H](F)C6)nc4c3F)c12] | 2-amino-4-(6-chloro-4- ((1R,5S)-8-(cyclopropyl- methyl)-3,8-diazabicyclo- [3.2.1]octan-3-yl)-8-fluoro- 2-(((2R,7aS)-2-fluorotetra- hydro-1H-pyrrolizin- 7a(5H)-yl)methoxy)quin- azolin-7-yl)-7-fluorobenzo- [b]thiophene-3-carbonitrile | 694.3 |
| 501 | 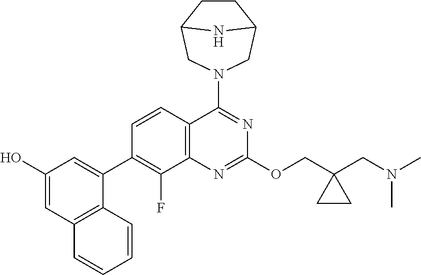 [SMILES: CN(C)CC1(COc2nc(N3CC4CCC(C3)N4)c3ccc(-c4cc(O)cc5ccccc45)c(F)c3n2)CC1] | 4-(4-(3,8-diazabicyclo- [3.2.1]octan-3-yl)-2-((1- ((dimethylamino)methyl)- cyclopropyl)methoxy)-8- fluoroquinazolin-7-yl)naph- thalen-2-ol | 528.5 |
| 502 | 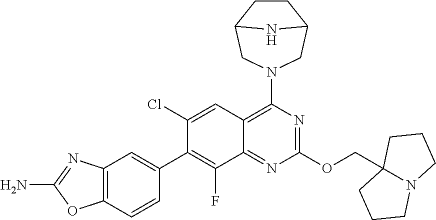 [SMILES: Nc1nc2cc(-c3c(Cl)cc4c(N5CC6CCC(C5)N6)nc(OCC56CCCN5CCC6)nc4c3F)ccc2o1] | 5-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)benzo- [d]oxazol-2-amine | 564.1 |
| 503 | 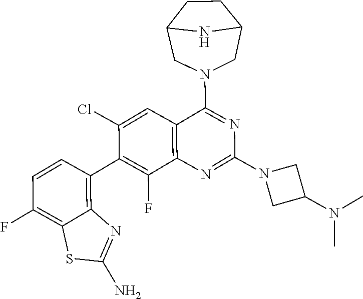 [SMILES: CN(C)C1CN(c2nc(N3CC4CCC(C3)N4)c3cc(Cl)c(-c4ccc(F)c5sc(N)nc45)c(F)c3n2)C1] | 4-(4-(3,8-diazabicyclo- [3.2.1]octan-3-yl)-6- chloro-2-(3-(dimethyl- amino)azetidin-1-yl)-8- fluoroquinazolin-7-yl)- 7-fluorobenzo[d]thiazol- 2-amine | 557.3 |
| 504 | 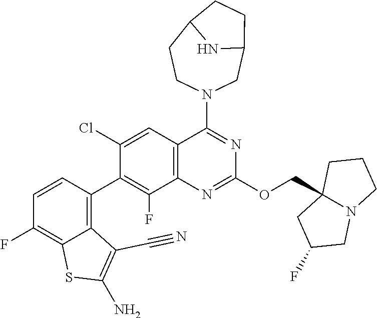 [SMILES: N#Cc1c(N)sc2c(F)ccc(-c3c(Cl)cc4c(N5CCC6CCC(C5)N6)nc(OC[C@@]56CCCN5C[C@H](F)C6)nc4c3F)c12] | 4-(4-(3,9-diazabicyclo- [4.2.1]nonan-3-yl)-6-chloro- 8-fluoro-2-(((2R,7aS)-2- fluorotetrahydro-1H-pyrrol- izin-7a(5H)-yl)methoxy)- quin-azolin-7-yl)-2-amino- 7-fluorobenzo[b]thiophene- 3-carbonitrile | 654.4 |
| 505 | 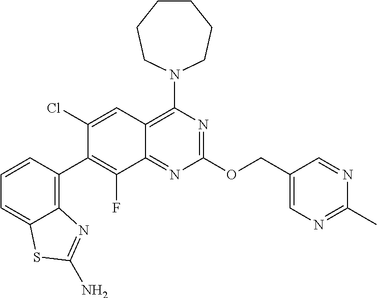 [SMILES: Cc1ncc(COc2nc(N3CCCCCC3)c3cc(Cl)c(-c4cccc5sc(N)nc45)c(F)c3n2)cn1] | 4-(4-(azepan-1-yl)-6-chloro- 8-fluoro-2-((2-methylpyr- imidin-5-yl)methoxy)quin- azolin-7-yl)benzo[d]thiazol- 2-amine | 551 |
| 506 | 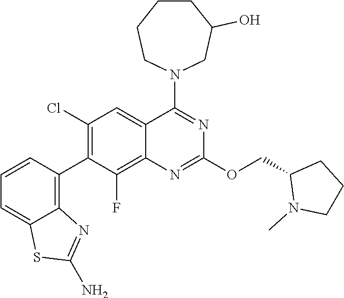 [SMILES: CN1CCC[C@H]1COc1nc(N2CCCCC(O)C2)c2cc(Cl)c(-c3cccc4sc(N)nc34)c(F)c2n1] | 1-(7-(2-aminobenzo[d]thia- zol-4-yl)-6-chloro-8-fluoro- 2-(((S)-1-methylpyrrolidin- 2-yl)methoxy)quinazolin-4- yl)azepan-3-ol | 557.3 |
| 507 | 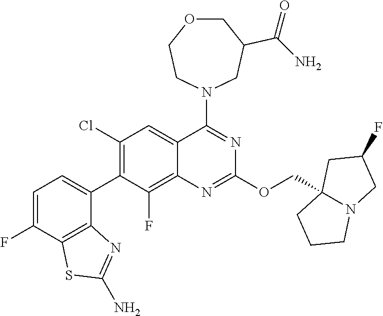 [SMILES: NC(=O)C1COCCN(c2nc(OC[C@@]34CCCN3C[C@H](F)C4)nc3c(F)c(-c4ccc(F)c5sc(N)nc45)c(Cl)cc23)C1] | 4-(7-(2-amino-7-fluoroben- zo[d]thiazol-4-yl)-6-chloro- 8-fluoro-2-(((2R,7aS)-2- fluorotetrahydro-1H-pyrrol- izin-7a(5H)-yl)methoxy)- quinazolin-4-yl)-1,4- oxazepane-6-carboxamide | 648.3 |
| 508 | 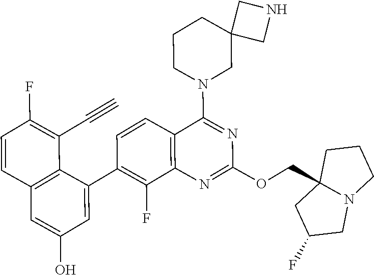 [SMILES: C#Cc1c(F)ccc2cc(O)cc(-c3ccc4c(N5CCCC6(CNC6)C5)nc(OC[C@@]56CCCN5C[C@H](F)C6)nc4c3F)c12] | 5-ethynyl-6-fluoro-4-(8- fluoro-2-(((2R,7aS)-2- fluorotetrahydro-1H-pyrrol- izin-7a(5H)-yl)methoxy)-4- (2,6-diazaspiro[3.5]nonan- 6-yl)quinazolin-7-yl)naph- thalen-2-ol | 614.3 |
| 509 | 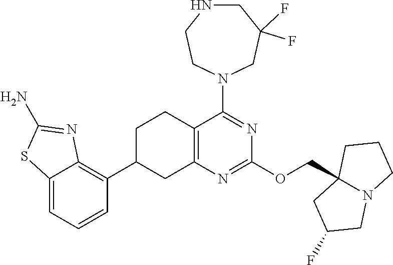 [SMILES: Nc1nc2c(C3CCc4c(nc(OC[C@@]56CCCN5C[C@H](F)C6)nc4N4CCNCC(F)(F)C4)C3)cccc2s1] | 4-(4-(6,6-difluoro-1,4- diazepan-1-yl)-2-(((2R,7aS)- 2-fluorotetrahydro-1H- pyrrolizin-7a(5H)-yl)meth- oxy)-5,6,7,8-tetrahydro- quinazolin-7-yl)benzo[d]- thiazol-2-amine | 574.3 |
| 510 | 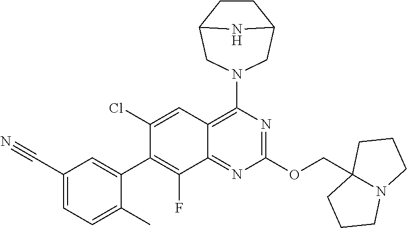 [SMILES: Cc1ccc(C#N)cc1-c1c(Cl)cc2c(N3CC4CCC(C3)N4)nc(OCC34CCCN3CCC4)nc2c1F] | 3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-4- methylbenzonitrile | 547.2 |
| 511 | 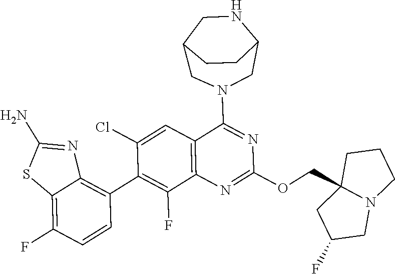 [SMILES: Nc1nc2c(-c3c(Cl)cc4c(N5CC6CCC(C5)NC6)nc(OC[C@@]56CCCN5C[C@H](F)C6)nc4c3F)ccc(F)c2s1] | 4-(4-(3,6-diazabicyclo- [3.2.2]nonan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 630.6 |
| 512 | 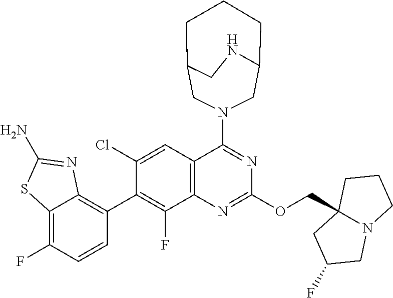 [SMILES: Nc1nc2c(-c3c(Cl)cc4c(N5CC6CCCC(C5)NC6)nc(OC[C@@]56CCCN5C[C@H](F)C6)nc4c3F)ccc(F)c2s1] | 4-(4-(3,9-diazabicyclo- [3.3.2]decan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 646.2 |
| 513 | 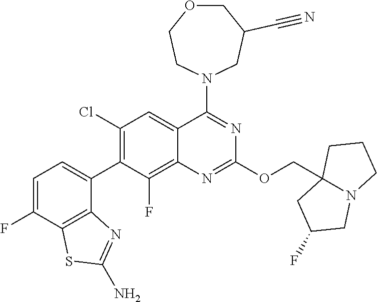 [SMILES: N#CC1COCCN(c2nc(OCC34CCCN3C[C@H](F)C4)nc3c(F)c(-c4ccc(F)c5sc(N)nc45)c(Cl)cc23)C1] | 4-(7-(2-amino-7-fluoroben- zo[d]thiazol-4-yl)-6-chloro- 8-fluoro-2-(((2R)-2-fluoro- tetrahydro-1H-pyrrolizin- 7a(5H)-yl)methoxy)quin- azolin-4-yl)-1,4-oxazepane- 6-carbonitrile | 630.1 |
| 514 | 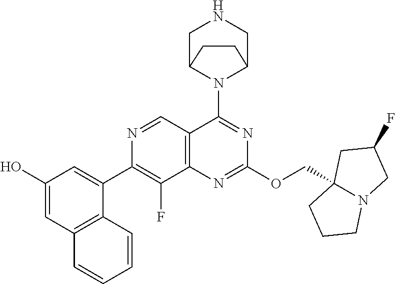 [SMILES: Oc1cc(-c2ncc3c(N4C5CCC4CNC5)nc(OC[C@@]45CCCN4C[C@H](F)C5)nc3c2F)c2ccccc2c1] | 4-(4-((1R,5S)-3,8-diazabi- cyclo-[3.2.1]octan-8-yl)-8- fluoro-2-(((2R,7aS)-2- fluorotetrahydro-1H-pyrrol- izin-7a(5H)-yl)methoxy)- pyrido[4,3-d]pyrimidin-7- yl)naphthalen-2-ol | 559.5 |
| 515 | 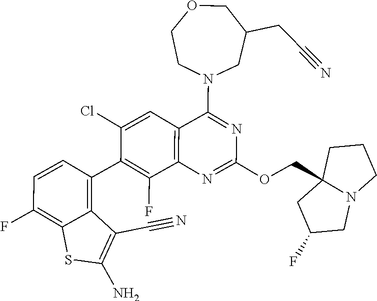 [SMILES: N#CCC1COCCN(c2nc(OC[C@@]34CCCN3C[C@H](F)C4)nc3c(F)c(-c4ccc(F)c5sc(N)c(C#N)c45)c(Cl)cc23)C1] | 2-amino-4-(6-chloro-4- (6-(cyanomethyl)-1,4- oxazepan-4-yl)-8-fluoro- 2-(((2R,7aS)-2-fluorotetra- hydro-1H-pyrrolizin-7a(5H)- yl)methoxy)quinazolin-7- yl)-7-fluorobenzo[b]- thiophene-3-carbonitrile | 668.2 |
| 516 | 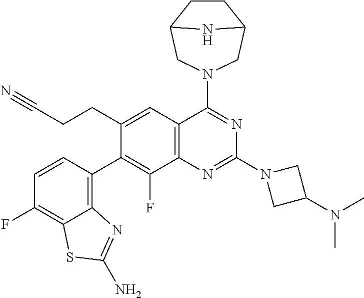 [SMILES: CN(C)C1CN(c2nc(N3CC4CCC(C3)N4)c3cc(CCC#N)c(-c4ccc(F)c5sc(N)nc45)c(F)c3n2)C1] | 3-(7-(2-amino-7-fluoroben- zo[d]thiazol-4-yl)-4-(3,8- diazabicyclo[3.2.1]octan-3- yl)-2-(3-(dimethylamino)- azetidin-1-yl)-8-fluoroquin- azolin-6-yl)propanenitrile | 576.4 |
| 517 | 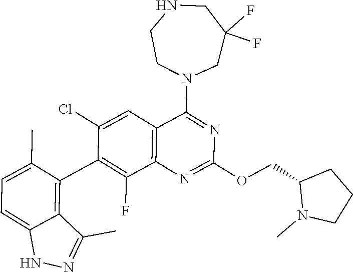 [SMILES: Cc1ccc2[nH]nc(C)c2c1-c1c(Cl)cc2c(N3CCNCC(F)(F)C3)nc(OC[C@@H]3CCCN3C)nc2c1F] | 6-chloro-4-(6,6-difluoro- 1,4-diazepan-1-yl)-7-(3,5- dimethyl-1H-indazol-4-yl)- 8-fluoro-2-(((S)-1-methyl- pyrrolidin-2-yl)methoxy)- quinazoline | 574.5 |
| 518 | 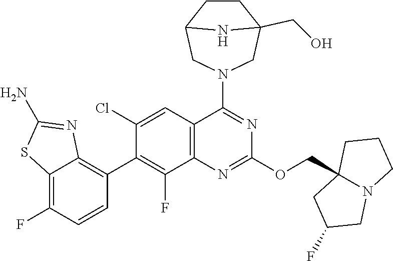 [SMILES: Nc1nc2c(-c3c(Cl)cc4c(N5CC6CCC(CO)(C5)N6)nc(OC[C@@]56CCCN5C[C@H](F)C6)nc4c3F)ccc(F)c2s1] | (3-(7-(2-amino-7-fluoroben- zo-[d]thiazol-4-yl)-6-chloro- 8-fluoro-2-(((2R,7aS)-2- fluorotetrahydro-1H-pyrrol- izin-7a(5H)-yl)methoxy)- quinazolin-4-yl)-3,8-diaza- bicyclo[3.2.1]octan-1-yl)- methanol | 646.5 |
| 519 | 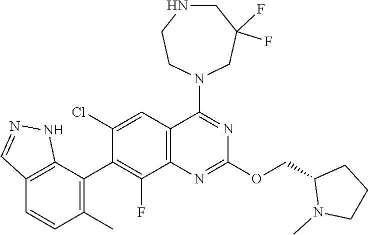 [SMILES: Cc1ccc2cn[nH]c2c1-c1c(Cl)cc2c(N3CCNCC(F)(F)C3)nc(OC[C@@H]3CCCN3C)nc2c1F] | 6-chloro-4-(6,6-difluoro-1,4- diazepan-1-yl)-8-fluoro-7- (6-methyl-1H-indazol-7-yl)- 2-(((S)-1-methylpyrrolidin- 2-yl)methoxy)quinazoline | 560.3 |
| 520 | 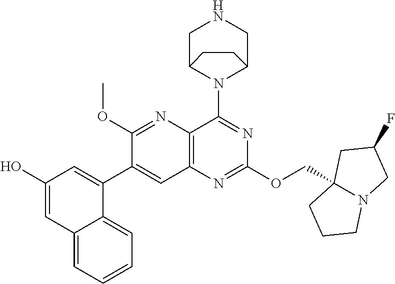 [SMILES: COc1nc2c(N3C4CCC3CNC4)nc(OC[C@@]34CCCN3C[C@H](F)C4)nc2cc1-c1cc(O)cc2ccccc12] | 4-(4-((1R,5S)-3,8-diaza- bicyclo-[3.2.1]octan-8-yl)- 2-(((2R,7aS)-2-fluorotetra- hydro-1H-pyrrolizin-7a(5H)- yl)methoxy)-6-methoxy- pyrido-[3,2-d]pyrimidin-7- yl)naphthalen-2-ol | 571.3 |
| 521 | 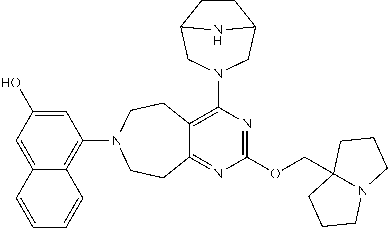 [SMILES: Oc1cc(N2CCc3nc(OCC45CCCN4CCC5)nc(N4CC5CCC(C4)N5)c3CC2)c2ccccc2c1] | 4-(4-(3,8-diazabicyclo- [3.2.1]octan-3-yl)-2-((tetra- hydro-1H-pyrrolizin-7a(5H)- yl)methoxy)-5,6,8,9-tetra- hydro-7H-pyrimido[4,5-d]- azepin-7-yl)naphthalen-2-ol | 541.6 |
| 522 | 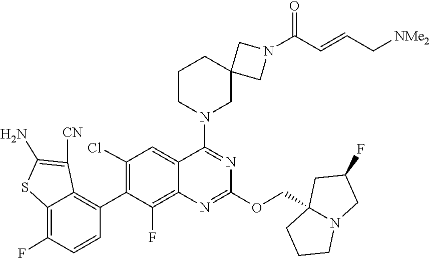 [SMILES: CN(C)C/C=C/C(=O)N1CC2(CCCN(c3nc(OC[C@@]45CCCN4C[C@H](F)C5)nc4c(F)c(-c5ccc(F)c6sc(N)c(C#N)c56)c(Cl)cc34)C2)C1] | 2-amino-4-(6-chloro-4-(2- ((E)-4-(dimethylamino)but- 2-enoyl)-2,6-diazaspiro- [3.5]nonan-6-yl)-8-fluoro- 2-(((2R,7aS)-2-fluorotetra- hydro-1H-pyrrolizin-7a(5H)- yl)methoxy)quinazolin-7- yl)-7-fluorobenzo[b]-thio- phene-3-carbonitrile | 765.2 |
| 523 | 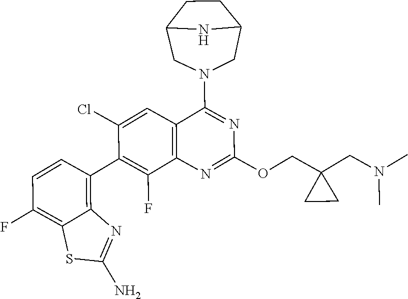 [SMILES: CN(C)CC1(COc2nc(N3CC4CCC(C3)N4)c3cc(Cl)c(-c4ccc(F)c5sc(N)nc45)c(F)c3n2)CC1] | 4-(4-(3,8-diazabicyclo- [3.2.1]octan-3-yl)-6-chloro- 2-((1-((dimethylamino)- methyl)cyclopropyl)meth- oxy)-8-fluoroquinazolin-7- yl)-7-fluorobenzo[d]thiazol- 2-amine | 586.5 |
| 524 | 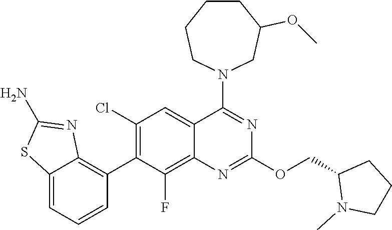 [SMILES: COC1CCCCN(c2nc(OC[C@@H]3CCCN3C)nc3c(F)c(-c4cccc5sc(N)nc45)c(Cl)cc23)C1] | 4-(6-chloro-8-fluoro-4-(3- methoxyazepan-1-yl)-2- (((S)-1-methylpyrrolidin-2- yl)methoxy)quinazolin-7- yl)benzo[d]thiazol-2-amine | 571.3 |
| 525 | 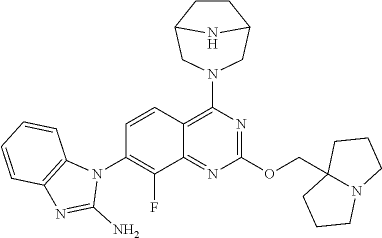 [SMILES: Nc1nc2ccccc2n1-c1ccc2c(N3CC4CCC(C3)N4)nc(OCC34CCCN3CCC4)nc2c1F] | 1-(4-(3,8-diazabicyclo- [3.2.1]octan-3-yl)-8-fluoro- 2-((tetrahydro-1H-pyrrol- izin-7a(5H)-yl)methoxy)- quin-azolin-7-yl)-1H-benzo- [d]imidazol-2-amine | 529.3 |
| 526 | 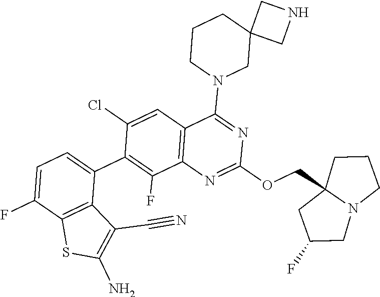 [SMILES: N#Cc1c(N)sc2c(F)ccc(-c3c(Cl)cc4c(N5CCCC6(CNC6)C5)nc(OC[C@@]56CCCN5C[C@H](F)C6)nc4c3F)c12] | 2-amino-4-(6-chloro-8- fluoro-2-(((2R,7aS)-2- fluorotetrahydro-1H-pyrrol- izin-7a(5H)-yl)methoxy)-4- (2,6-diazaspiro[3.5]nonan- 6-yl)quinazolin-7-yl)-7- fluorobenzo[b]thiophene- 3-carbonitrile | 654.3 |
| 527 | 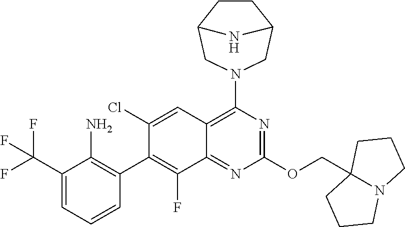 [SMILES: Nc1c(-c2c(Cl)cc3c(N4CC5CCC(C4)N5)nc(OCC45CCCN4CCC5)nc3c2F)cccc1C(F)(F)F] | 2-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-6- (trifluoromethyl)aniline | 591.3 |
| 528 | 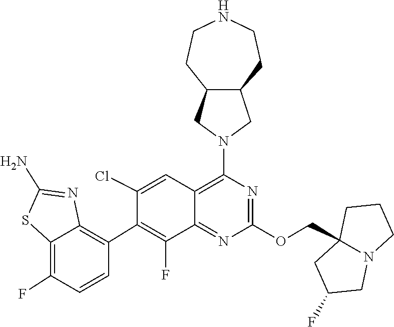 [SMILES: Nc1nc2c(-c3c(Cl)cc4c(N5C[C@H]6CCNCC[C@H]6C5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin- 7a(5H)-yl)methoxy)-4- ((3aR,8aS)-octahydropyr- rolo[3,4-d]azepin-2(1H)- yl)quinazolin-7-yl)-7- fluorobenzo[d]thiazol-2- amine | 645.4 |
| 529 | 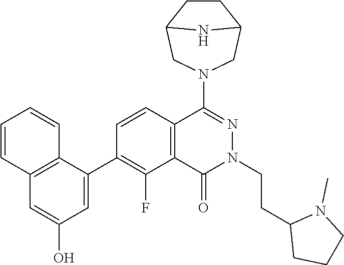 [SMILES: CN1CCCC1CCn1nc(N2CC3CCC(C2)N3)c2ccc(-c3cc(O)cc4ccccc34)c(F)c2c1=O] | 4-(3,8-diazabicyclo[3.2.1]- octan-3-yl)-8-fluoro-7-(3- hydroxynaphthalen-1-yl)-2- (2-(1-methylpyrrolidin-2- yl)ethyl)phthalazin-1(2H)- one | 528.2 |
| 530 | 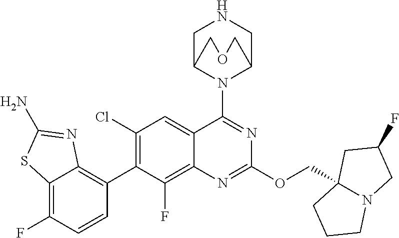 [SMILES: Nc1nc2c(-c3c(Cl)cc4c(N5C6CNCC5COC6)nc(OC[C@@]56CCCN5C[C@H](F)C6)nc4c3F)ccc(F)c2s1] | 4-(4-(3-oxa-7,9-diaza- bicyclo-[3.3.1]nonan-9-yl)- 6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 632.2 |
| 531 | 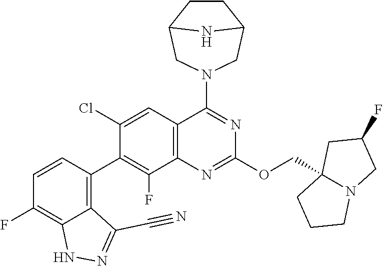 [SMILES: N#Cc1n[nH]c2c(F)ccc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quin-azolin-7-yl)-7-fluoro- 1H-indazole-3-carbonitrile | 607.4 [M − H]− |
| 532 | 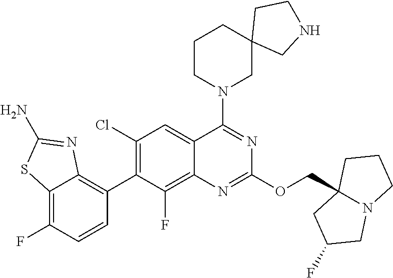 [SMILES: Nc1nc2c(-c3c(Cl)cc4c(N5CCCC6(CCNC6)C5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin-7a(5H)- yl)methoxy)-4-(2,7-diaza- spiro[4.5]decan-7-yl)quin- azolin-7-yl)-7-fluorobenzo- [d]thiazol-2-amine | 644.1 |
| 533 | 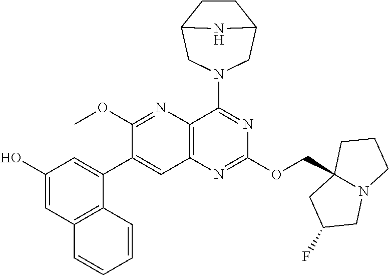 [SMILES: COc1nc2c(N3CC4CCC(C3)N4)nc(OC[C@@]34CCCN3C[C@H](F)C4)nc2cc1-c1cc(O)cc2ccccc12] | 4-(4-(3,8-diazabicyclo- [3.2.1]octan-3-yl)-2- (((2R,7aS)-2-fluorotetra- hydro-1H-pyrrolizin- 7a(5H)-yl)methoxy)-6- methoxypyrido-[3,2-d]- pyrimidin-7-yl)naphthalen- 2-ol | 571.4 |
| 534 | 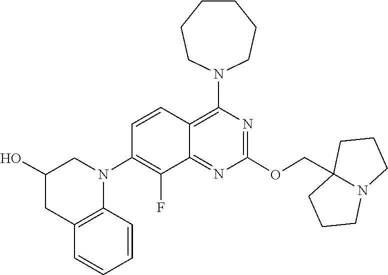 [SMILES: OC1Cc2ccccc2N(c2ccc3c(N4CCCCCC4)nc(OCC45CCCN4CCC5)nc3c2F)C1] | 1-(4-(azepan-1-yl)-8-fluoro- 2-((tetrahydro-1H-pyrrol- izin-7a(5H)-yl)methoxy)- quinazolin-7-yl)-1,2,3,4- tetrahydroquinolin-3-ol | 532.3 |
| 535 | 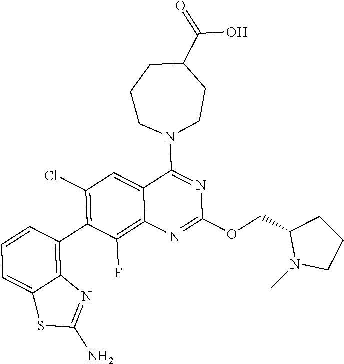 [SMILES: CN1CCC[C@H]1COc1nc(N2CCCC(C(=O)O)CC2)c2cc(Cl)c(-c3cccc4sc(N)nc34)c(F)c2n1] | 1-(7-(2-aminobenzo[d]thia- zol-4-yl)-6-chloro-8-fluoro- 2-(((S)-1-methylpyrrolidin- 2-yl)methoxy)quinazolin-4- yl)azepane-4-carboxylic acid | 585.5 |
| 536 | 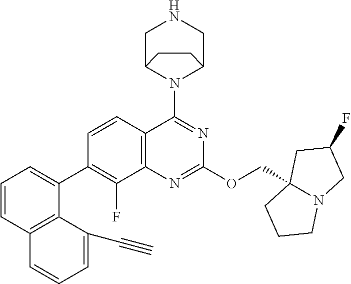 [SMILES: C#Cc1cccc2cccc(-c3ccc4c(N5C6CCC5CNC6)nc(OC[C@@]56CCCN5C[C@H](F)C6)nc4c3F)c12] | 4-(4-((1R,5S)-3,8-diaza- bicyclo-[3.2.1]octan-8-yl)- 8-fluoro-2-(((2R,7aS)-2- fluorotetrahydro-1H-pyrrol- izin-7a(5H)-yl)methoxy)- quinazolin-7-yl)-5-ethynyl- naphthalen-2-ol | 582.5 |
| 537 | 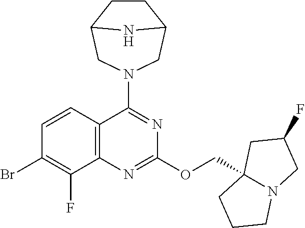 [SMILES: Fc1c(Br)ccc2c(N3CC4CCC(C3)N4)nc(OC[C@@]34CCCN3C[C@H](F)C4)nc12] | 4-(3,8-diazabicyclo[3.2.1]- octan-3-yl)-7-bromo-8- fluoro-2-(((2R,7aS)-2- fluorotetrahydro-1H-pyrrol- izin-7a(5H)-yl)methoxy)- quinazoline | 496.2 |
| 538 | 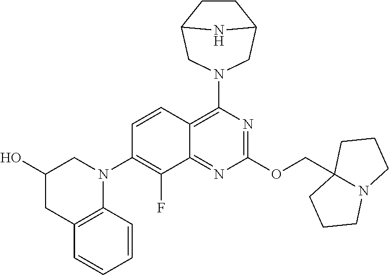 [SMILES: OC1Cc2ccccc2N(c2ccc3c(N4CC5CCC(C4)N5)nc(OCC45CCCN4CCC5)nc3c2F)C1] | 1-(4-(3,8-diazabicyclo- [3.2.1]octan-3-yl)-8-fluoro- 2-((tetrahydro-1H-pyrrol- izin-7a(5H)-yl)methoxy)- quinazolin-7-yl)-1,2,3,4- tetrahydro-quinolin-3-ol | 545.3 |
| 539 | 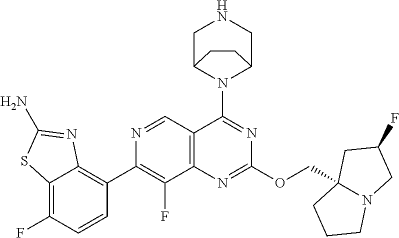 [SMILES: Nc1nc2c(-c3ncc4c(N5C6CCC5CNC6)nc(OC[C@@]56CCCN5C[C@H](F)C6)nc4c3F)ccc(F)c2s1] | 4-(4-((1R,5S)-3,8-diaza- bicyclo[3.2.1]octan-8-yl)- 8-fluoro-2-(((2R,7aS)-2- fluorotetrahydro-1H-pyrrol- izin-7a(5H)-yl)methoxy)- pyrido[4,3-d]pyrimidin-7- yl)-7-fluorobenzo[d]thiazol- 2-amine | 583.4 |
| 540 | 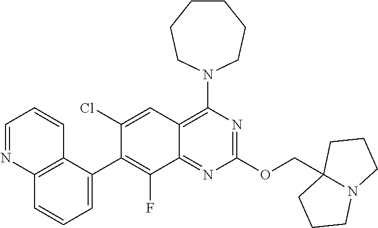 [SMILES: Fc1c(-c2cccc3ncccc23)c(Cl)cc2c(N3CCCCCC3)nc(OCC34CCCN3CCC4)nc12] | 4-(azepan-1-yl)-6-chloro-8- fluoro-7-(quinolin-5-yl)-2- ((tetrahydro-1H-pyrrolizin- 7a(5H)-yl)methoxy)quin- azoline | 547.3 |
| 541 | 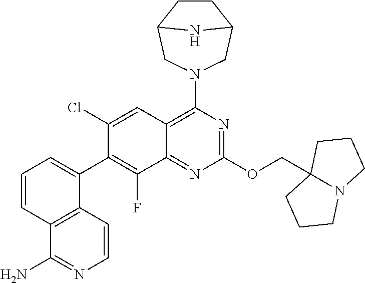 [SMILES: Nc1nccc2c(-c3c(Cl)cc4c(N5CC6CCC(C5)N6)nc(OCC56CCCN5CCC6)nc4c3F)cccc12] | 5-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)iso- quinolin-1-amine | 574.2 |
| 542 | 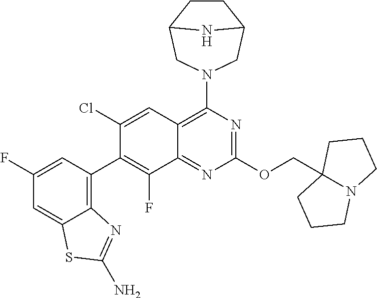 [SMILES: Nc1nc2c(-c3c(Cl)cc4c(N5CC6CCC(C5)N6)nc(OCC56CCCN5CCC6)nc4c3F)cc(F)cc2s1] | 4-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-6- fluorobenzo[d]thiazol-2- amine | 598.1 |
| 543 | 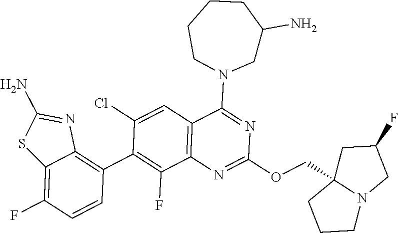 [SMILES: Nc1nc2c(-c3c(Cl)cc4c(N5CCCCC(N)C5)nc(OC[C@@]56CCCN5C[C@H](F)C6)nc4c3F)ccc(F)c2s1] | 4-(4-(3-aminoazepan-1-yl)- 6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 616.3 [M − H]− |
| 544 | 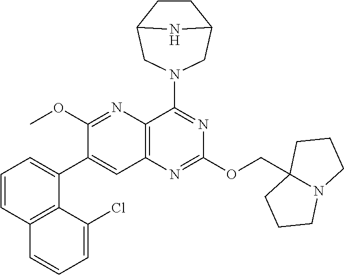 [SMILES: COc1nc2c(N3CC4CCC(C3)N4)nc(OCC34CCCN3CCC4)nc2cc1-c1cccc2cccc(Cl)c12] | 4-(3,8-diazabicyclo[3.2.1]- octan-3-yl)-7-(8-chloronaph- thalen-1-yl)-6-methoxy-2- ((tetrahydro-1H-pyrrolizin- 7a(5H)-yl)methoxy)pyrido- [3,2-d]pyrimidine | 571.4 |
| 545 | 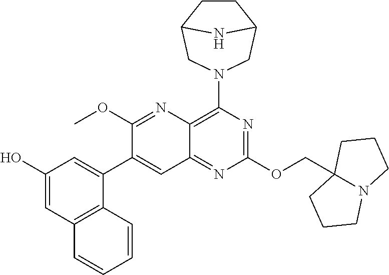 [SMILES: COc1nc2c(N3CC4CCC(C3)N4)nc(OCC34CCCN3CCC4)nc2cc1-c1cc(O)cc2ccccc12] | 4-(4-(3,8-diazabicyclo- [3.2.1]-octan-3-yl)-6-meth- oxy-2-((tetrahydro-1H- pyrrolizin-7a(5H)-yl)meth- oxy)pyrido[3,2-d]pyrimidin- 7-yl)naphthalen-2-ol | 553.4 |
| 546 | 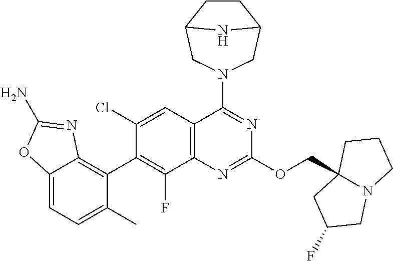 [SMILES: Cc1ccc2oc(N)nc2c1-c1c(Cl)cc2c(N3CC4CCC(C3)N4)nc(OC[C@@]34CCCN3C[C@H](F)C4)nc2c1F] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-5-methyl- benzo[d]oxazol-2-amine | 596.3 |
| 547 | 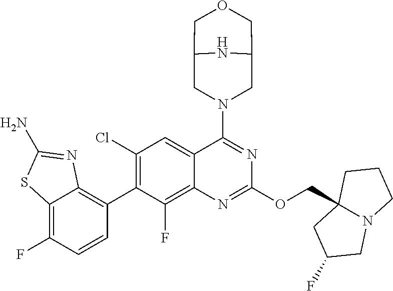 [SMILES: Nc1nc2c(-c3c(Cl)cc4c(N5CC6COCC(C5)N6)nc(OC[C@@]56CCCN5C[C@H](F)C6)nc4c3F)ccc(F)c2s1] | 4-(4-(3-oxa-7,9-diaza- bicyclo[3.3.1]nonan-7-yl)- 6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 632.3 |
| 548 | 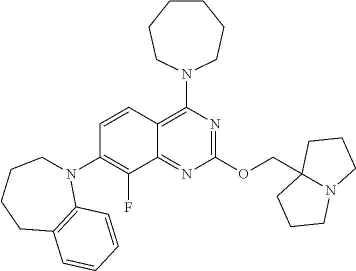 [SMILES: Fc1c(N2CCCCc3ccccc32)ccc2c(N3CCCCCC3)nc(OCC34CCCN3CCC4)nc12] | 1-(4-(azepan-1-yl)-8-fluoro- 2-((tetrahydro-1H-pyrrol- izin-7a(5H)-yl)methoxy)- quinazolin-7-yl)-2,3,4,5- tetrahydro-1H-benzo[b]- azepine | 530.4 |
| 549 | 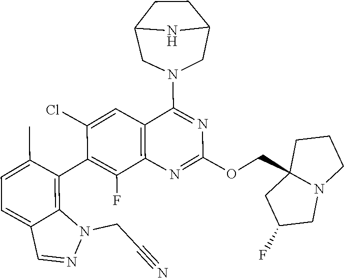 [SMILES: Cc1ccc2cnn(CC#N)c2c1-c1c(Cl)cc2c(N3CC4CCC(C3)N4)nc(OC[C@@]34CCCN3C[C@H](F)C4)nc2c1F] | 2-(7-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-6-methyl- 1H-indazol-1-yl)acetonitrile | 619.3 |
| 550 | 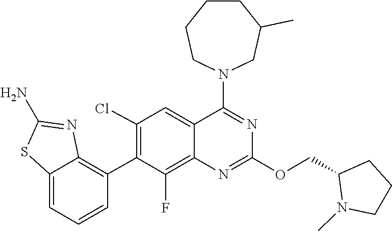 [SMILES: CC1CCCCN(c2nc(OC[C@@H]3CCCN3C)nc3c(F)c(-c4cccc5sc(N)nc45)c(Cl)cc23)C1] | 4-(6-chloro-8-fluoro-4-(3- methylazepan-1-yl)-2-(((S)- 1-methylpyrrolidin-2-yl)- methoxy)quinazolin-7-yl)- benzo[d]thiazol-2-amine | 555.2 |
| 551 | 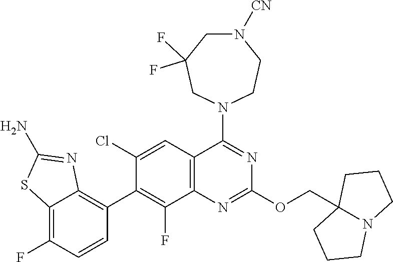 [SMILES: N#CN1CCN(c2nc(OCC34CCCN3CCC4)nc3c(F)c(-c4ccc(F)c5sc(N)nc45)c(Cl)cc23)CC(F)(F)C1] | 4-(7-(2-amino-7-fluoroben- zo[d]thiazol-4-yl)-6-chloro- 8-fluoro-2-((tetrahydro-1H- pyrrolizin-7a(5H)-yl)meth- oxy)quinazolin-4-yl)-6,6- difluoro-1,4-diazepane-1- carbonitrile | 648.2 |
| 552 | 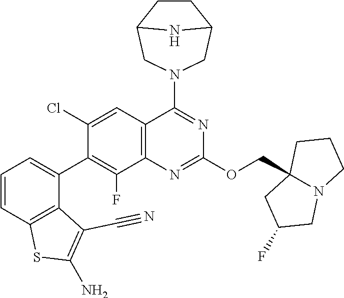 [SMILES: N#Cc1c(N)sc2cccc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-2-amino- benzo[b]thiophene-3- carbonitrile | 622.2 |
| 553 | 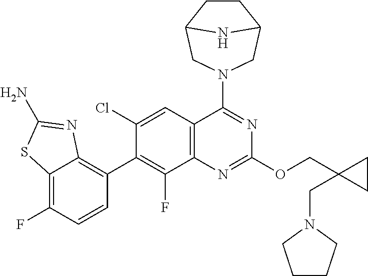 [SMILES: Nc1nc2c(-c3c(Cl)cc4c(N5CC6CCC(C5)N6)nc(OCC5(CN6CCCC6)CC5)nc4c3F)ccc(F)c2s1] | 4-(4-(3,8-diazabicyclo- [3.2.1]-octan-3-yl)-6-chloro- 8-fluoro-2-((1-(pyrrolidin-1- ylmethyl)cyclopropyl)meth- oxy)quinazolin-7-yl)-7- fluorobenzo[d]thiazol-2- amine | 612.5 |
| 554 | 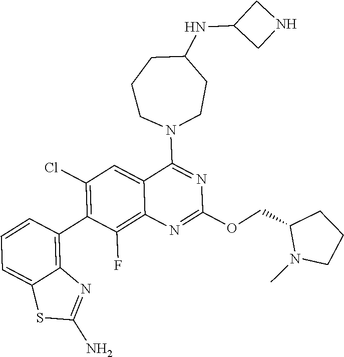 [SMILES: CN1CCC[C@H]1COc1nc(N2CCCC(NC3CNC3)CC2)c2cc(Cl)c(-c3cccc4sc(N)nc34)c(F)c2n1] | 4-(4-(4-(azetidin-3-yl- amino)azepan-1-yl)-6- chloro-8-fluoro-2-(((S)-1- methylpyrrolidin-2-yl)- methoxy)quinazolin-7-yl)- benzo[d]thiazol-2-amine | 611.3 |
| 555 | 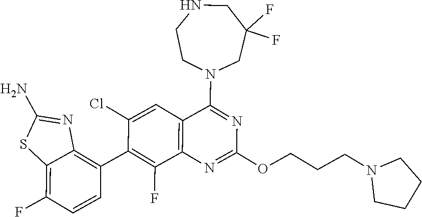 [SMILES: Nc1nc2c(-c3c(Cl)cc4c(N5CCNCC(F)(F)C5)nc(OCCCN5CCCC5)nc4c3F)ccc(F)c2s1] | 4-(6-chloro-4-(6,6-difluoro- 1,4-diazepan-1-yl)-8-fluoro- 2-(3-(pyrrolidin-1-yl)- propoxy)quinazolin-7-yl)- 7-fluorobenzo[d]thiazol-2- amine | 610.6 |
| 556 | 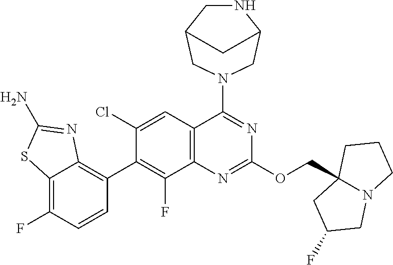 [SMILES: Nc1nc2c(-c3c(Cl)cc4c(N5CC6CNC(C6)C5)nc(OC[C@@]56CCCN5C[C@H](F)C6)nc4c3F)ccc(F)c2s1] | 4-(4-(3,6-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 616.,4 |
| 557 | 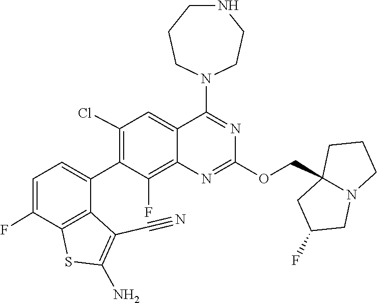 [SMILES: N#Cc1c(N)sc2c(F)ccc(-c3c(Cl)cc4c(N5CCCNCC5)nc(OC[C@@]56CCCN5C[C@H](F)C6)nc4c3F)c12] | 2-amino-4-(6-chloro-4-(1,4- diazepan-1-yl)-8-fluoro-2- (((2R,7aS)-2-fluorotetra- hydro-1H-pyrrolizin-7a(5H)- yl)methoxy)quinazolin-7- yl)-7-fluorobenzo[b]thio- phene-3-carbonitrile | 628.4 |
| 558 | 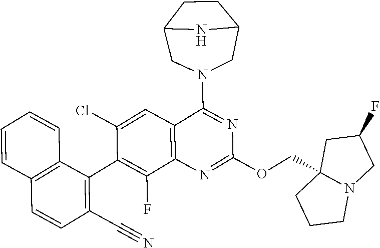 [SMILES: N#Cc1ccc2ccccc2c1-c1c(Cl)cc2c(N3CC4CCC(C3)N4)nc(OC[C@@]34CCCN3C[C@H](F)C4)nc2c1F] | 1-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-2-naph- thonitrile | 601.4 |
| 559 | 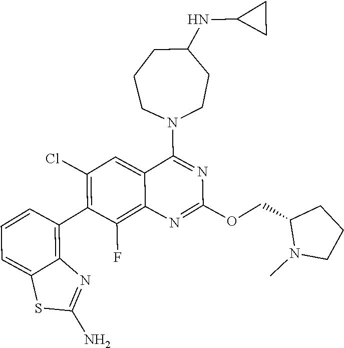 [SMILES: CN1CCC[C@H]1COc1nc(N2CCCC(NC3CC3)CC2)c2cc(Cl)c(-c3cccc4sc(N)nc34)c(F)c2n1] | 4-(6-chloro-4-(4-(cyclo- propylamino)azepan-1-yl)- 8-fluoro-2-(((S)-1-methyl- pyrrolidin-2-yl)methoxy)- quinazolin-7-yl)benzo[d]- thiazol-2-amine | 596.3 |
| 560 | 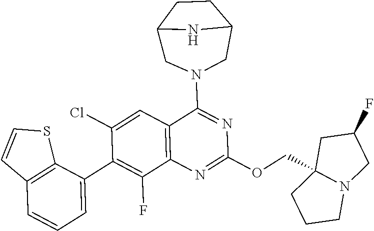 [SMILES: Fc1c(-c2cccc3ccsc23)c(Cl)cc2c(N3CC4CCC(C3)N4)nc(OC[C@@]34CCCN3C[C@H](F)C4)nc12] | 7-(benzo[b]thiophen-7-yl)- 4-(3,8-diazabicyclo[3.2.1]- octan-3-yl)-6-chloro-8- fluoro-2-(((2R,7aS)-2- fluorotetrahydro-1H-pyrrol- izin-7a(5H)-yl)methoxy)- quinazoline | 582.3 |
| 561 | 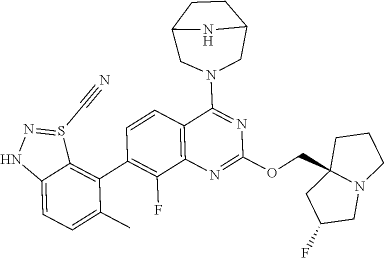 [SMILES: Cc1ccc2c(c1-c1ccc3c(N4CC5CCC(C4)N5)nc(OC[C@@]45CCCN4C[C@H](F)C5)nc3c1F)S(C#N)=NN2] | 4-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-5-methyl-1H-indazole- 3-carbonitrile | 571.5 |
| 562 | 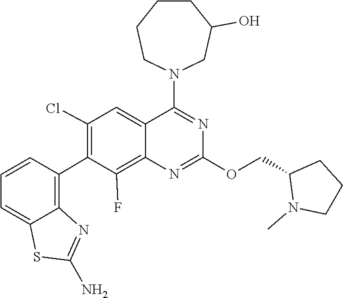 [SMILES: CN1CCC[C@H]1COc1nc(N2CCCCC(O)C2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)azepan-3- ol | 557.3 |
| 563 | 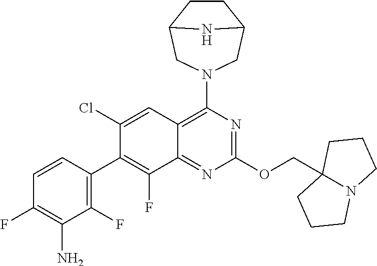 [SMILES: Nc1c(F)ccc(-c2c(Cl)cc3c(N4CC5CCC(C4)N5)nc(OCC45CCCN4CCC5)nc3c2F)c1F] | 3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-2,6- difluoroaniline | 559.2 |
| 564 | 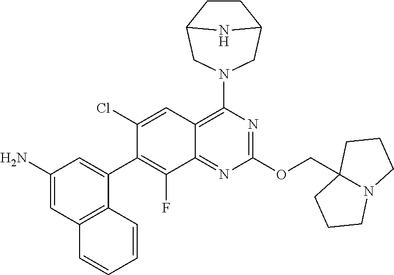 [SMILES: Nc1cc(-c2c(Cl)cc3c(N4CC5CCC(C4)N5)nc(OCC45CCCN4CCC5)nc3c2F)c2ccccc2c1] | 4-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)naph- thalen-2-amine | 573.4 |
| 565 | 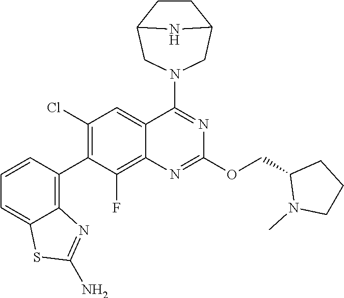 [SMILES: CN1CCC[C@H]1COc1nc(N2CC3CCC(C2)N3)c2cc(Cl)c(-c3cccc4sc(N)nc34)c(F)c2n1] | 4-(4-(3,8-diazabicyclo- [3.2.1]octan-3-yl)-6-chloro- 8-fluoro-2-(((S)-1-methyl- pyrrolidin-2-yl)methoxy)- quinazolin-7-yl)benzo[d]- thiazol-2-amine | 554.2 |
| 566 | 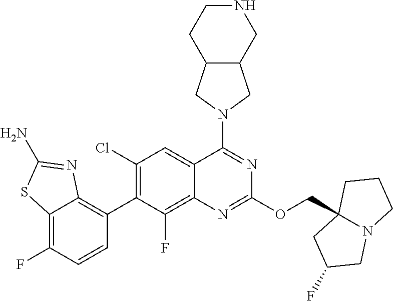 [SMILES: Nc1nc2c(-c3c(Cl)cc4c(N5CC6CCNCC6C5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin-7a(5H)- yl)methoxy)-4-(octahydro- 2H-pyrrolo[3,4-c]pyridin-2- yl)quinazolin-7-yl)-7-fluoro- benzo[d]thiazol-2-amine | 630.2 |
| 567 | 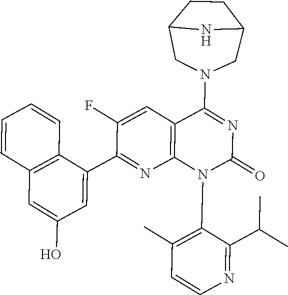 [SMILES: Cc1ccnc(C(C)C)c1-n1c(=O)nc(N2CC3CCC(C2)N3)c2cc(F)c(-c3cc(O)cc4ccccc34)nc21] | 4-(3,8-diazabicyclo[3.2.1]- octan-3-yl)-6-fluoro-7-(3- hydroxynaphthalen-1-yl)-1- (2-isopropyl-4-methylpyri- din-3-yl)pyrido[2,3-d]pyrim- idin-2(1H)-one | 551.5 |
| 568 | 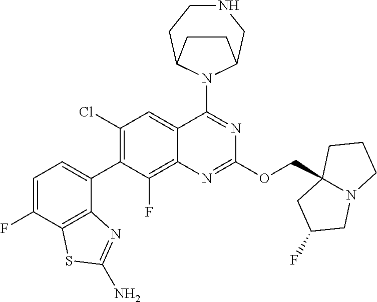 [SMILES: Nc1nc2c(-c3c(Cl)cc4c(N5C6CCNCC5CC6)nc(OC[C@@]56CCCN5C[C@H](F)C6)nc4c3F)ccc(F)c2s1] | 4-(4-(3,9-diazabicyclo- [4.2.1]-nonan-9-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 630.7 |
| 569 | 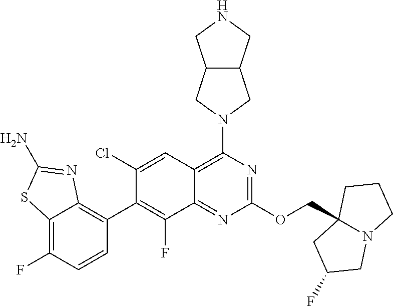 [SMILES: Nc1nc2c(-c3c(Cl)cc4c(N5CC6CNCC6C5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin-7a(5H)- yl)methoxy)-4-(hexahydro- pyrrolo[3,4-c]pyrrol-2(1H)- yl)quinazolin-7-yl)-7-fluoro- benzo[d]thiazol-2-amine | 616 |
| 570 | 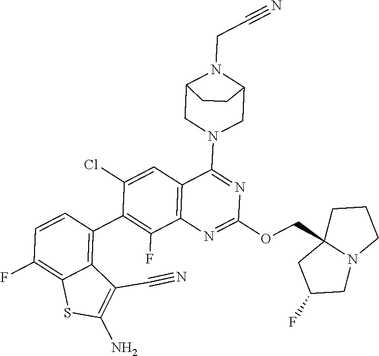 [SMILES: N#CCN1C2CCC1CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4- ((1R,5S)-8-(cyanomethyl)- 3,8-diazabicyclo[3.2.1]- octan-3-yl)-8-fluoro-2- (((2R,7aS)-2-fluorotetra- hydro-1H-pyrrolizin-7a(5H)- yl)methoxy)quinazolin-7- yl)-7-fluorobenzo[b]thio- phene-3-carbonitrile | 679.1 |
| 571 | 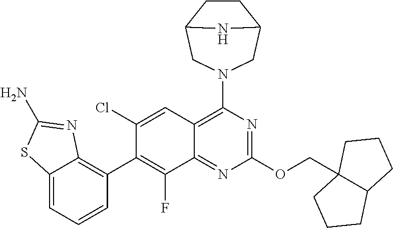 [SMILES: Nc1nc2c(-c3c(Cl)cc4c(N5CC6CCC(C5)N6)nc(OCC56CCCC5CCC6)nc4c3F)cccc2s1] | 4-(4-(3,8-diazabicyclo- [3.2.1]octan-3-yl)-6-chloro- 8-fluoro-2-((hexahydro- pentalen-3a(1H)-yl)meth- oxy)quinazolin-7-yl)benzo- [d]thiazol-2-amine | 579.2 |
| 572 | 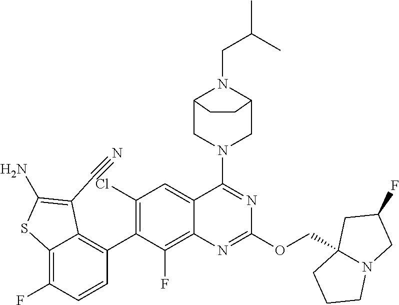 [SMILES: CC(C)CN1C2CCC1CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8- fluoro-2-(((2R,7aS)-2- fluorotetrahydro-1H-pyrrol- izin-7a(5H)-yl)methoxy)- 4-((1R,5S)-8-isobutyl-3,8- diazabicyclo[3.2.1]octan-3- yl)quinazolin-7-yl)-7- fluorobenzo[b]thiophene- 3-carbonitrile | 696.2 |
| 573 | 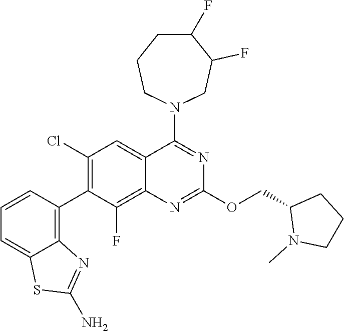 [SMILES: CN1CCC[C@H]1COc1nc(N2CCCC(F)C(F)C2)c2cc(Cl)c(-c3cccc4sc(N)nc34)c(F)c2n1] | 4-(6-chloro-4-(3,4-difluoro- azepan-1-yl)-8-fluoro-2- (((S)-1-methylpyrrolidin-2- yl)methoxy)quinazolin-7-yl)- benzo[d]thiazol-2-amine | 577.3 |
| 574 | 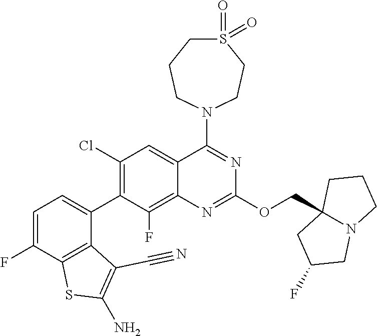 [SMILES: N#Cc1c(N)sc2c(F)ccc(-c3c(Cl)cc4c(N5CCCS(=O)(=O)CC5)nc(OC[C@@]56CCCN5C[C@H](F)C6)nc4c3F)c12] | 2-amino-4-(6-chloro-4-(1,1- dioxido-1,4-thiazepan-4-yl)- 8-fluoro-2-(((2R,7aS)-2- fluorotetrahydro-1H-pyrrol- izin-7a(5H)-yl)methoxy)- quinazolin-7-yl)-7-fluoro- benzo[b]thiophene-3- carbonitrile | 677.3 |
| 575 | 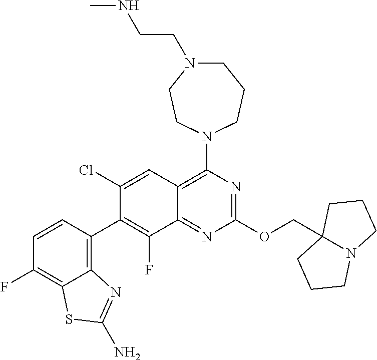 [SMILES: CNCCN1CCCN(c2nc(OCC34CCCN3CCC4)nc3c(F)c(-c4ccc(F)c5sc(N)nc45)c(Cl)cc23)CC1] | 4-(6-chloro-8-fluoro-4-(4- (2-(methylamino)ethyl)-1,4- diazepan-1-yl)-2-((tetra- hydro-1H-pyrrolizin-7a(5H)- yl)methoxy)quinazolin-7- yl)-7-fluorobenzo[d]thiazol- 2-amine | 634.4 |
| 576 | 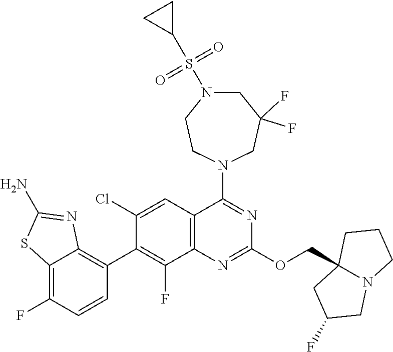 [SMILES: Nc1nc2c(-c3c(Cl)cc4c(N5CCN(S(=O)(=O)C6CC6)CC(F)(F)C5)nc(OC[C@@]56CCCN5C[C@H](F)C6)nc4c3F)ccc(F)c2s1] | 4-(6-chloro-4-(4-cycloprop- yl-sulfonyl)-6,6-difluoro-1,4- diazepan-1-yl)-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 742.4 [M − H]− |
| 577 | 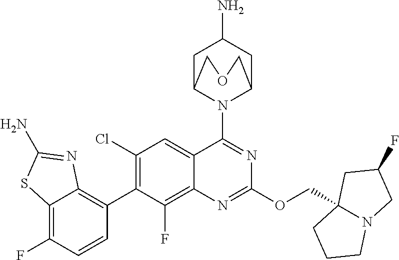 [SMILES: Nc1nc2c(-c3c(Cl)cc4c(N5C6COCC5CC(N)C6)nc(OC[C@@]56CCCN5C[C@H](F)C6)nc4c3F)ccc(F)c2s1] | 9-(7-(2-amino-7-fluoroben- zo[d]thiazol-4-yl)-6-chloro- 8-fluoro-2-(((2R,7aS)-2- fluorotetrahydro-1H-pyrrol- izin-7a(5H)-yl)methoxy)- quinazolin-4-yl)-3-oxa-9- azabicyclo[3.3.1]nonan-7- amine | 646.4 |
| 578 | 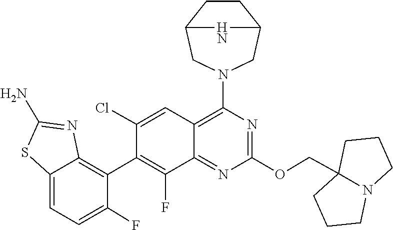 [SMILES: Nc1nc2c(-c3c(Cl)cc4c(N5CC6CCC(C5)N6)nc(OCC56CCCN5CCC6)nc4c3F)c(F)ccc2s1] | 4-(4-(3,8-diazabicyclo- [3.2.1]octan-3-yl)-6-chloro- 8-fluoro-2-((tetrahydro-1H- pyrrolizin-7a(5H)-l)meth- oxy)quinazolin-7-yl)-5- fluorobenzo[d]thiazol-2- amine | 598.2 |
| 579 | 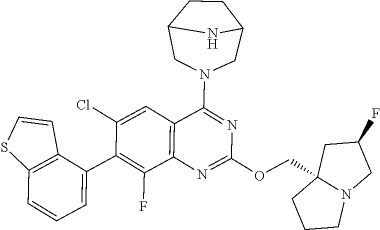 [SMILES: Fc1c(-c2cccc3sccc23)c(Cl)cc2c(N3CC4CCC(C3)N4)nc(OC[C@@]34CCCN3C[C@H](F)C4)nc12] | 7-(benzo[b]thiophen-4-yl)- 4-(3,8-diazabicyclo[3.2.1]- octan-3-yl)-6-chloro-8- fluoro-2-(((2R,7aS)-2- fluorotetrahydro-1H-pyrrol- izin-7a(5H)-yl)methoxy)- quinazoline | 582.3 |
| 580 | 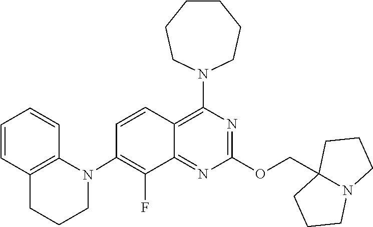 [SMILES: Fc1c(N2CCCc3ccccc32)ccc2c(N3CCCCCC3)nc(OCC34CCCN3CCC4)nc12] | 4-(azepan-1-yl)-7-(3,4- dihydroquinolin-1(2H)-yl)- 8-fluoro-2-((tetrahydro-1H- pyrrolizin-7a(5H)-yl)meth- oxy)quinazoline | 516.3 |
| 581 | 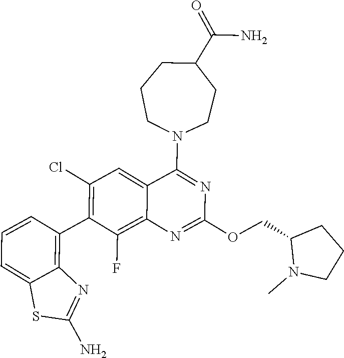 [SMILES: CN1CCC[C@H]1COc1nc(N2CCCC(C(N)=O)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)azepane- 4-carboxamide | 584.3 |
| 582 | 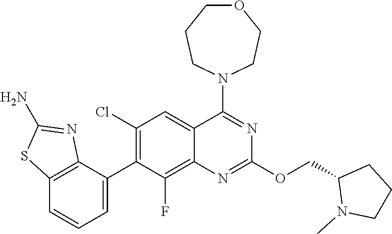 [SMILES: CN1CCC[C@H]1COc1nc(N2CCCOCC2)c2cc(Cl)c(-c3cccc4sc(N)nc34)c(F)c2n1] | 4-(6-chloro-8-fluoro-2-(((S)- 1-methylpyrrolidin-2-yl)- methoxy)-4-(1,4-oxazepan- 4-yl)quinazolin-7-yl)benzo- [d]thiazol-2-amine | 544.5 |
| 583 | 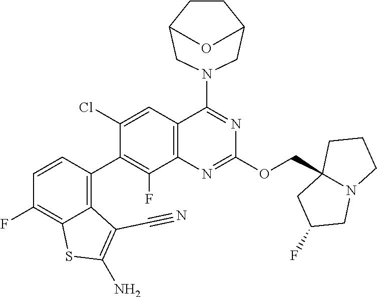 [SMILES: N#Cc1c(N)sc2c(F)ccc(-c3c(Cl)cc4c(N5CC6CCC(C5)O6)nc(OC[C@@]56CCCN5C[C@H](F)C6)nc4c3F)c12] | 4-(4-(8-oxa-3-azabicyclo- [3.2.1]-octan-3-yl)-6-chloro- 8-fluoro-2-(((2R,7aS)-2- fluorotetrahydro-1H-pyrrol- izin-7a(5H)-yl)methoxy)- quinazolin-7-yl)-2-amino- 7-fluorobenzo[b]thiophene- 3-carbonitrile | 641.5 |
| 584 | 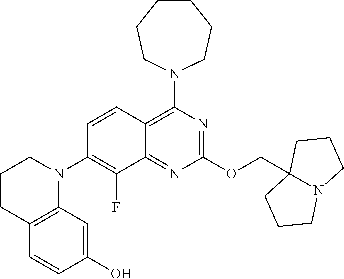 [SMILES: Oc1ccc2c(c1)N(c1ccc3c(N4CCCCCC4)nc(OCC45CCCN4CCC5)nc3c1F)CCC2] | 1-(4-(azepan-1-yl)-8-fluoro- 2-((tetrahydro-1H-pyrrol- izin-7a(5H)-yl)methoxy)- quinazolin-7-yl)-1,2,3,4- tetrahydroquinolin-7-ol | 533.3 |
| 585 | 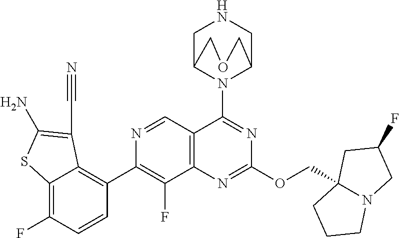 [SMILES: N#Cc1c(N)sc2c(F)ccc(-c3ncc4c(N5C6CNCC5COC6)nc(OC[C@@]56CCCN5C[C@H](F)C6)nc4c3F)c12] | 4-(4-(3-oxa-7,9-diaza- bicyclo[3.3.1]nonan-9-yl)- 8-fluoro-2-(((2R,7aS)-2- fluorotetrahydro-1H- pyrrolizin-7a(5H)-yl)meth- oxy)pyrido[4,3-d]pyrimidin- 7-yl)-2-amino-7-fluoroben- zo[b]thiophene-3-carbo- nitrile | 623.4 |
| 586 | 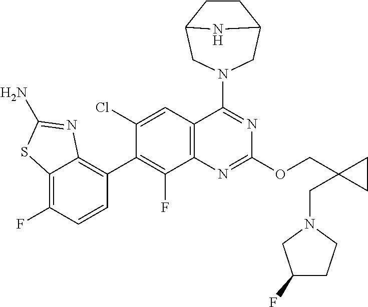 [SMILES: Nc1nc2c(-c3c(Cl)cc4c(N5CC6CCC(C5)N6)nc(OCC5(CN6CC[C@@H](F)C6)CC5)nc4c3F)ccc(F)c2s1] | 4-(4-(3,8-diazabicyclo- [3.2.1]octan-3-yl)-6-chloro- 8-fluoro-2-((1-(((R)-3- fluoropyrrolidin-1-yl)meth- yl)cyclopropyl)methoxy)- quinazolin-7-yl)-7-fluoro- benzo[d]thiazol-2-amine | 630.4 |
| 587 | 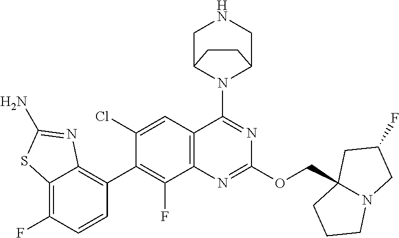 [SMILES: Nc1nc2c(-c3c(Cl)cc4c(N5C6CCC5CNC6)nc(OC[C@]56CCCN5C[C@@H](F)C6)nc4c3F)ccc(F)c2s1] | 4-(4-((1R,5S)-3,8-diaza- bicyclo-[3.2.1]octan-8-yl)- 6-chloro-8-fluoro-2- (((2S,7aR)-2-fluorotetra- hydro-1H-pyrrolizin- 7a(5H)-yl)methoxy)quin- azolin-7-yl)-7-fluorobenzo- [d]thiazol-2-amine | 616.5 |
| 588 | 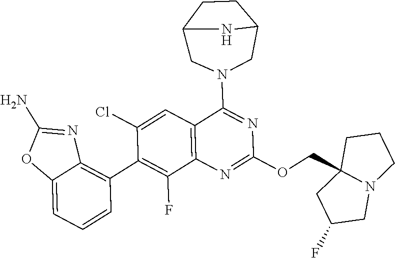 [SMILES: Nc1nc2c(-c3c(Cl)cc4c(N5CC6CCC(C5)N6)nc(OC[C@@]56CCCN5C[C@H](F)C6)nc4c3F)cccc2o1] | 4-(4-(3,8-diazabicyclo- [3.2.1]-octan-3-yl)-6-chloro- 8-fluoro-2-(((2R,7aS)-2- fluorotetrahydro-1H-pyrrol- izin-7a(5H)-yl)methoxy)- quinazolin-7-yl)benzo[d]- oxazol-2-amine | 580.4 [M − H]− |
| 589 | 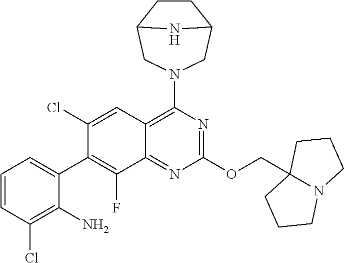 [SMILES: Nc1c(Cl)cccc1-c1c(Cl)cc2c(N3CC4CCC(C3)N4)nc(OCC34CCCN3CCC4)nc2c1F] | 2-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-6- chloroaniline | 557.3 |
| 590 | 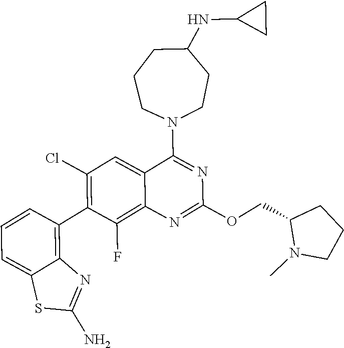 [SMILES: CN1CCC[C@H]1COc1nc(N2CCCC(NC3CC3)CC2)c2cc(Cl)c(-c3cccc4sc(N)nc34)c(F)c2n1] | 4-(6-chloro-4-(4-(cycloprop- ylamino)azepan-1-yl)-8- fluoro-2-(((S)-1-methylpyr- rolidin-2-yl)methoxy)quin- azolin-7-yl)benzo[d]thiazol- 2-amine | 596.3 |
| 591 | 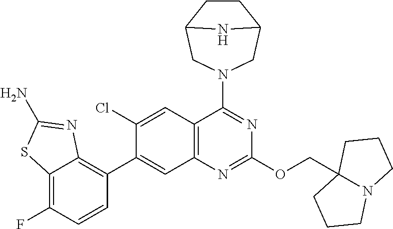 [SMILES: Nc1nc2c(-c3cc4nc(OCC56CCCN5CCC6)nc(N5CC6CCC(C5)N6)c4cc3Cl)ccc(F)c2s1] | 4-(4-(3,8-diazabicyclo- [3.2.1]octan-3-yl)-6-chloro- 2-((tetrahydro-1H-pyrrol- izin-7a(5H)-yl)methoxy)- quinazolin-7-yl)-7-fluoro- benzo[d]thiazol-2-amine | 580.3 |
| 592 | 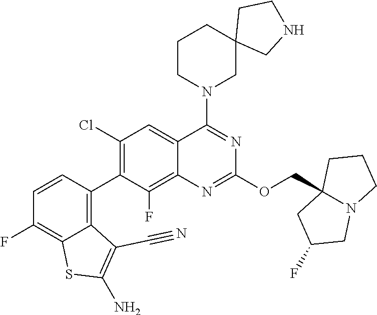 [SMILES: N#Cc1c(N)sc2c(F)ccc(-c3c(Cl)cc4c(N5CCCC6(CCNC6)C5)nc(OC[C@@]56CCCN5C[C@H](F)C6)nc4c3F)c12] | 2-amino-4-(6-chloro-8- fluoro-2-(((2R,7aS)-2- fluorotetrahydro-1H-pyrrol- izin-7a(5H)-yl)methoxy)-4- (2,7-diazaspiro[4.5]decan- 7-yl)quinazolin-7-yl)-7- fluorobenzo[b]thiophene- 3-carbonitrile | 668.3 |
| 593 | 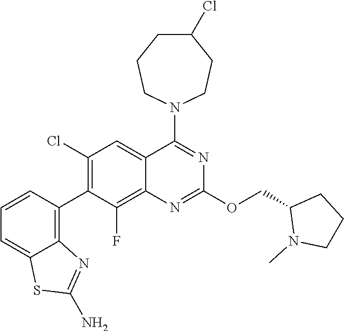 [SMILES: CN1CCC[C@H]1COc1nc(N2CCCC(Cl)CC2)c2cc(Cl)c(-c3cccc4sc(N)nc34)c(F)c2n1] | 4-(6-chloro-4-(4-chloro- azepan-1-yl)-8-fluoro-2- (((S)-1-methylpyrrolidin-2- yl)methoxy)quinazolin-7- yl)benzo[d]thiazol-2-amine | 575.1 |
| 594 | 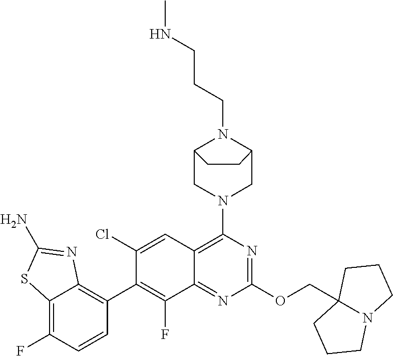 [SMILES: CNCCCN1C2CCC1CN(c1nc(OCC34CCCN3CCC4)nc3c(F)c(-c4ccc(F)c5sc(N)nc45)c(Cl)cc13)C2] | 4-(6-chloro-8-fluoro-4- ((1R,5S)-8-(3-(methyl- amino)propyl)-3,8-diaza- bicyclo[3.2.1]octan-3-yl)- 2-((tetrahydro-1H-pyrrol- izin-7a(5H)-yl)methoxy)- quinazolin-7-yl)-7-fluoro- benzo[d]thiazol-2-amine | 669.3 |
| 595 | 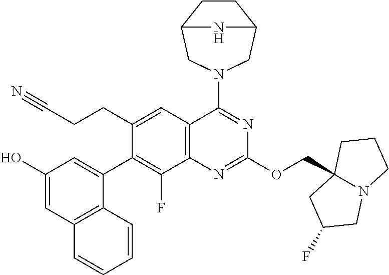 [SMILES: N#CCCc1cc2c(N3CC4CCC(C3)N4)nc(OC[C@@]34CCCN3C[C@H](F)C4)nc2c(F)c1-c1cc(O)cc2ccccc12] | 3-(4-(3,8-diazabicyclo- [3.2.1]octan-3-yl)-8-fluoro- 2-(((2R,7aS)-2-fluorotetra- hydro-1H-pyrrolizin- 7a(5H)-yl)methoxy)-7-(3- hydroxynaphthalen-1-yl)- quinazolin-6-yl)propane- nitrile | 611.4 |
| 596 | 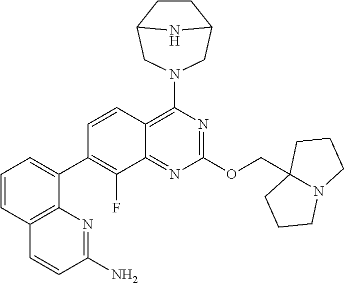 [SMILES: Nc1ccc2cccc(-c3ccc4c(N5CC6CCC(C5)N6)nc(OCC56CCCN5CCC6)nc4c3F)c2n1] | 8-(4-(3,8-diazabicyclo- [3.2.1]octan-3-yl)-8-fluoro- 2-((tetrahydro-1H-pyrrol- izin-7a(5H)-yl)methoxy)- quinazolin-7-yl)quinolin- 2-amine | 540.2 |
| 597 | 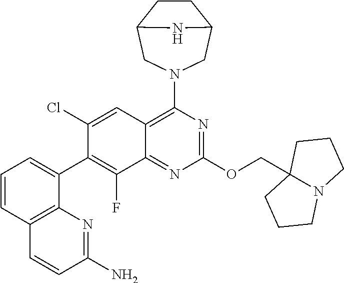 [SMILES: Nc1ccc2cccc(-c3c(Cl)cc4c(N5CC6CCC(C5)N6)nc(OCC56CCCN5CCC6)nc4c3F)c2n1] | 8-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)quin- olin-2-amine | 574.3 |
| 598 | 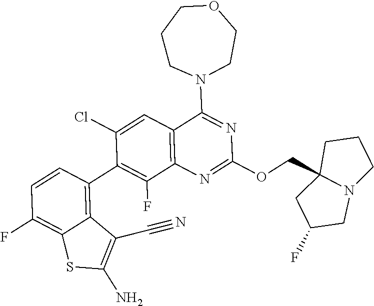 [SMILES: N#Cc1c(N)sc2c(F)ccc(-c3c(Cl)cc4c(N5CCCOCC5)nc(OC[C@@]56CCCN5C[C@H](F)C6)nc4c3F)c12] | 2-amino-4-(6-chloro-8- fluoro-2-(((2R,7aS)-2- fluorotetrahydro-1H-pyrrol- izin-7a(5H)-yl)methoxy)-4- (1,4-oxazepan-4-yl)quin- azolin-7-yl)-7-fluorobenzo- [b]thiophene-3-carbonitrile | 629.4 |
| 599 | 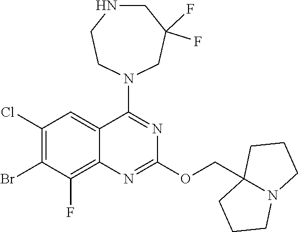 [SMILES: Fc1c(Br)c(Cl)cc2c(N3CCNCC(F)(F)C3)nc(OCC34CCCN3CCC4)nc12] | 7-bromo-6-chloro-4-(6,6- difluoro-1,4-diazepan-1-yl)- 8-fluoro-2-((tetrahydro-1H- pyrrolizin-7a(5H)-yl)meth- oxy)quinazoline | 534.1 |
| 600 | 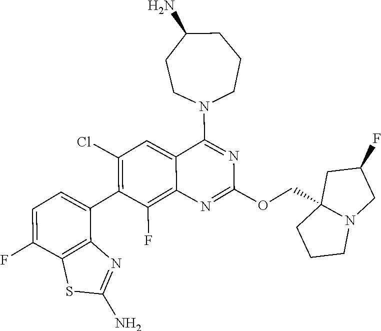 [SMILES: Nc1nc2c(-c3c(Cl)cc4c(N5CCC[C@H](N)CC5)nc(OC[C@@]56CCCN5C[C@H](F)C6)nc4c3F)ccc(F)c2s1] | 4-(4-((S)-4-aminoazepan-1- yl)-6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 618.8 |
| 601 | 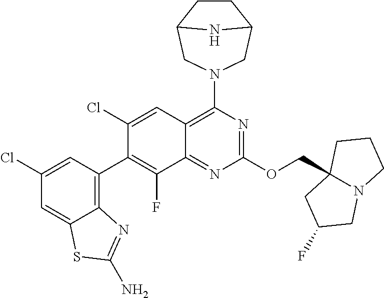 [SMILES: Nc1nc2c(-c3c(Cl)cc4c(N5CC6CCC(C5)N6)nc(OC[C@@]56CCCN5C[C@H](F)C6)nc4c3F)cc(Cl)cc2s1] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-6-chloro- benzo[d]thiazol-2-amine | 632.1 |
| 602 | 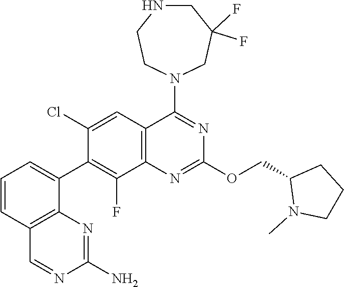 [SMILES: CN1CCC[C@H]1COc1nc(N2CCNCC(F)(F)C2)c2cc(Cl)c(-c3cccc4cnc(N)nc34)c(F)c2n1] | 6-chloro-4-(6,6-difluoro- 1,4-diazepan-1-yl)-8-fluoro- 2-(((S)-1-methylpyrrolidin- 2-yl)methoxy)-[7,8′-biquin- azolin]-2′-amine | 573.8 |
| 603 | 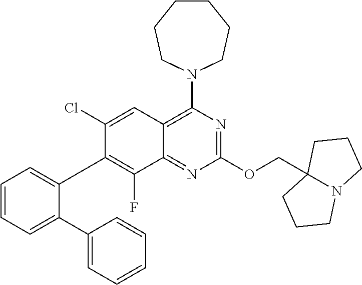 [SMILES: Fc1c(-c2ccccc2-c2ccccc2)c(Cl)cc2c(N3CCCCCC3)nc(OCC34CCCN3CCC4)nc12] | 7-([1,1′-biphenyl]-2-yl)-4- (azepan-1-yl)-6-chloro-8- fluoro-2-((tetrahydro-1H- pyrrolizin-7a(5H)-yl)meth- oxy)quinazoline | 572.3 |
| 604 | 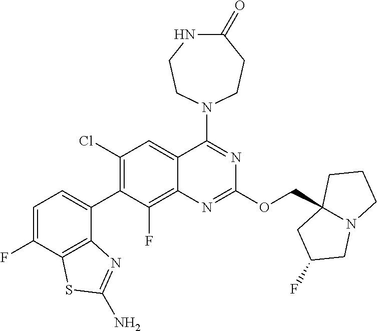 [SMILES: Nc1nc2c(-c3c(Cl)cc4c(N5CCNC(=O)CC5)nc(OC[C@@]56CCCN5C[C@H](F)C6)nc4c3F)ccc(F)c2s1] | 1-(7-(2-amino-7-fluoroben- zo[d]thiazol-4-yl)-6-chloro- 8-fluoro-2-(((2R,7aS)-2- fluorotetrahydro-1H-pyrrol- izin-7a(5H)-yl)methoxy)- quinazolin-4-yl)-1,4-diazepan-5-one | 618.1 |
| 605 | 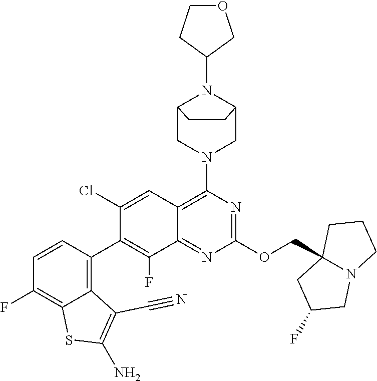 [SMILES: N#Cc1c(N)sc2c(F)ccc(-c3c(Cl)cc4c(N5CC6CCC(C5)N6C5CCOC5)nc(OC[C@@]56CCCN5C[C@H](F)C6)nc4c3F)c12] | 2-amino-4-(6-chloro-8- fluoro-2-(((2R,7aS)-2-fluoro- tetrahydro-1H-pyrrolizin- 7a(5H)-yl)methoxy)-4- ((1R,5S)-8-(tetrahydro- furan-3-yl)-3,8-diazabicyclo- [3.2.1]octan-3-yl)quinazolin- 7-yl)-7-fluorobenzo[b]thio- phene-3-carbonitrile | 710.2 |
| 606 | 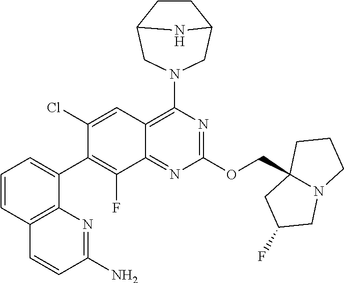 [SMILES: Nc1ccc2cccc(-c3c(Cl)cc4c(N5CC6CCC(C5)N6)nc(OC[C@@]56CCCN5C[C@H](F)C6)nc4c3F)c2n1] | 8-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)quinolin-2- amine | 592.2 |
| 607 | 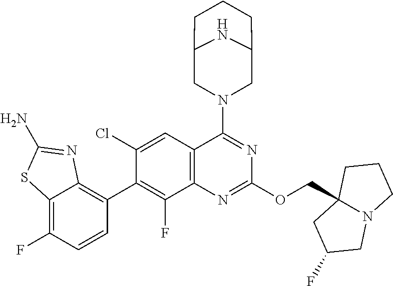 [SMILES: Nc1nc2c(-c3c(Cl)cc4c(N5CC6CCCC(C5)N6)nc(OC[C@@]56CCCN5C[C@H](F)C6)nc4c3F)ccc(F)c2s1] | 4-(4-(3,9-diazabicyclo- [3.3.1]nonan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 631.2 |
| 608 | 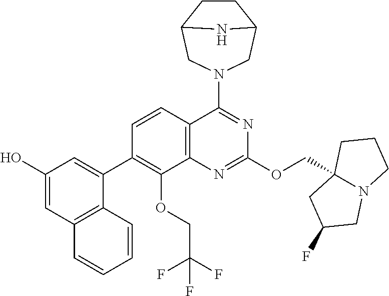 [SMILES: Oc1cc(-c2ccc3c(N4CC5CCC(C4)N5)nc(OC[C@]45CCCN4C[C@@H](F)C5)nc3c2OCC(F)(F)F)c2ccccc2c1] | 4-(4-(3,8-diazabicyclo- [3.2.1]octan-3-yl)-2- (((2S,7aR)-2-fluorotetra- hydro-1H-pyrrolizin- 7a(5H)-yl)methoxy)-8- (2,2,2-trifluoroethoxy)- quinazolin-7-yl)naph- thalen-2-ol | 638.5 |
| 609 | 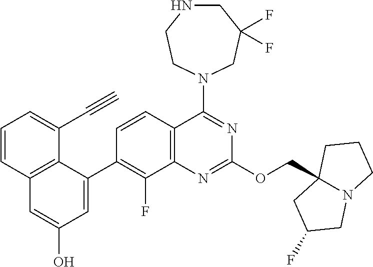 [SMILES: C#Cc1cccc2cc(O)cc(-c3ccc4c(N5CCNCC(F)(F)C5)nc(OC[C@@]56CCCN5C[C@H](F)C6)nc4c3F)c12] | 4-(4-(6,6-difluoro-1,4- diazepan-1-yl)-8-fluoro-2- (((2R,7aS)-2-fluorotetra- hydro-1H-pyrrolizin- 7a(5H)-yl)methoxy)quin- azolin-7-yl)-5-ethynylnaph- thalen-2-ol | 605.5 |
| 610 | 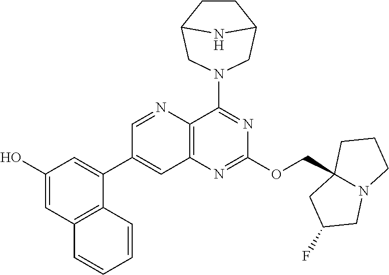 [SMILES: Oc1cc(-c2cnc3c(N4CC5CCC(C4)N5)nc(OC[C@@]45CCCN4C[C@H](F)C5)nc3c2)c2ccccc2c1] | 4-(4-(3,8-diazabicyclo- [3.2.1]octan-3-yl)-2- (((2R,7aS)-2-fluorotetra- hydro-1H-pyrrolizin-7a(5H)- yl)methoxy)pyrido[3,2-d]- pyrimidin-7-yl)naphthalen- 2-ol | 541.5 |
| 611 | 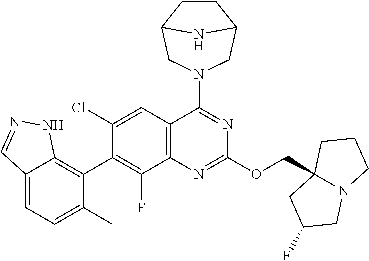 [SMILES: Cc1ccc2cn[nH]c2c1-c1c(Cl)cc2c(N3CC4CCC(C3)N4)nc(OC[C@@]34CCCN3C[C@H](F)C4)nc2c1F] | 4-(3,8-diazabicyclo[3.2.1]- octan-3-yl)-6-chloro-8- fluoro-2-(((2R,7aS)-2- fluorotetrahydro-1H-pyrrol- izin-7a(5H)-yl)methoxy)-7- (6-methyl-1H-indazol-7-yl)- quinazoline | 578.4 [M − H]− |
| 612 | 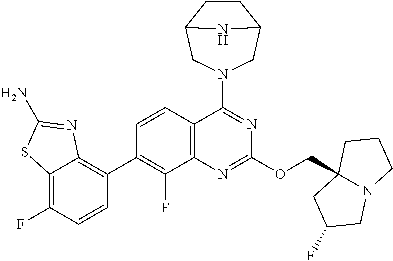 [SMILES: Nc1nc2c(-c3ccc4c(N5CC6CCC(C5)N6)nc(OC[C@@]56CCCN5C[C@H](F)C6)nc4c3F)ccc(F)c2s1] | 4-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-7-fluorobenzo[d]thiazol- 2-amine | 582.5 |
| 613 | 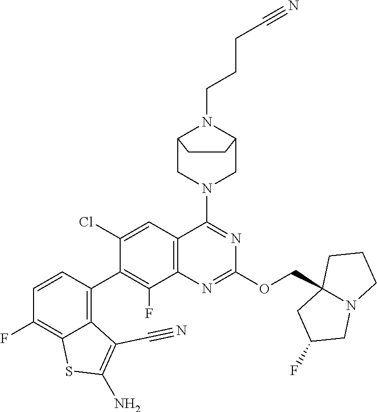 [SMILES: N#CCCCN1C2CCC1CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4- ((1R,5S)-8-(3-cyanoprop- yl)-3,8-diazabicyclo[3.2.1]- octan-3-yl)-8-fluoro-2- (((2R,7aS)-2-fluorotetra- hydro-1H-pyrrolizin-7a(5H)- yl)methoxy)quinazolin-7- yl)-7-fluorobenzo[b]thio- phene-3-carbonitrile | 707.2 |
| 614 | 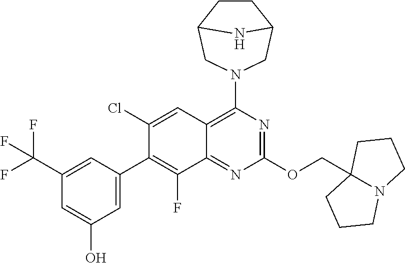 [SMILES: Oc1cc(-c2c(Cl)cc3c(N4CC5CCC(C4)N5)nc(OCC45CCCN4CCC5)nc3c2F)cc(C(F)(F)F)c1] | 3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-5- (trifluoromethyl)phenol | 592.2 |
| 615 | 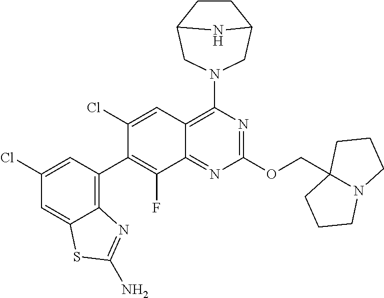 [SMILES: Nc1nc2c(-c3c(Cl)cc4c(N5CC6CCC(C5)N6)nc(OCC56CCCN5CCC6)nc4c3F)cc(Cl)cc2s1] | 4-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-6- chlorobenzo[d]thiazol-2- amine | 614.1 |
| 616 | 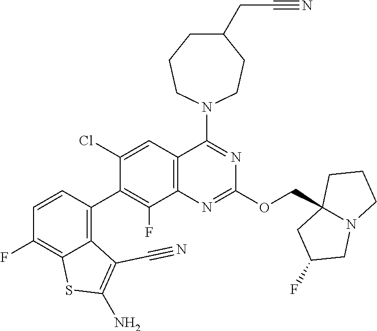 [SMILES: N#CCC1CCCN(c2nc(OC[C@@]34CCCN3C[C@H](F)C4)nc3c(F)c(-c4ccc(F)c5sc(N)c(C#N)c45)c(Cl)cc23)CC1] | 2-amino-4-(6-chloro-4-(4- (cyanomethyl)azepan-1-yl)- 8-fluoro-2-(((2R,7aS)-2- fluorotetrahydro-1H-pyrrol- izin-7a(5H)-yl)methoxy)- quinazolin-7-yl)-7-fluoro- benzo[b]thiophene-3- carbonitrile | 666.3 |
| 617 | 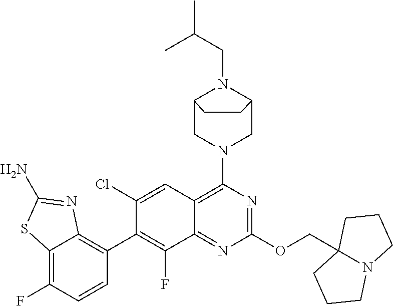 [SMILES: CC(C)CN1C2CCC1CN(c1nc(OCC34CCCN3CCC4)nc3c(F)c(-c4ccc(F)c5sc(N)nc45)c(Cl)cc13)C2] | 4-(6-chloro-8-fluoro-4- ((1R,5S)-8-isobutyl-3,8- diazabicyclo[3.2.1]octan- 3-yl)-2-((tetrahydro-1H- pyrrolizin-7a(5H)-yl)meth- oxy)quinazolin-7-yl)-7- fluorobenzo[d]thiazol-2- amine | 654.2 |
| 618 | 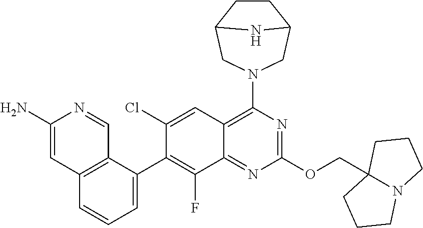 [SMILES: Nc1cc2cccc(-c3c(Cl)cc4c(N5CC6CCC(C5)N6)nc(OCC56CCCN5CCC6)nc4c3F)c2cn1] | 8-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)iso- quinolin-3-amine | 574.3 |
| 619 | 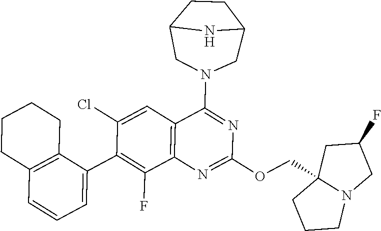 [SMILES: Fc1c(-c2cccc3c2CCCC3)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-6-chloro-8- fluoro-2-(((2R,7aS)-2- fluorotetrahydro-1H-pyrrol- izin-7a(5H)-yl)methoxy)- 7-(5,6,7,8-tetrahydronaph- thalen-1-yl)quinazoline | 580.3 |
| 620 | 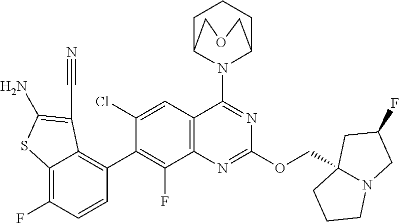 | 4-(4-(3-oxa-9-azabicyclo- [3.3.1]nonan-9-yl)-6-chloro- 8-fluoro-2-(((2R,7aS)-2- fluorotetrahydro-1H-pyrrol- izin-7a(5H)-yl)methoxy)- quinazolin-7-yl)-2-amino-7- fluorobenzo[b]thiophene-3- carbonitrile | 655.4 |
| 621 | 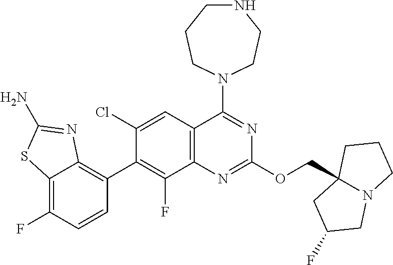 [SMILES: Nc1nc2c(-c3c(Cl)cc4c(N5CCCNCC5)nc(OC[C@@]56CCCN5C[C@H](F)C6)nc4c3F)ccc(F)c2s1] | 4-(6-chloro-4-(1,4-diazepan- 1-yl)-8-fluoro-2-(((2R,7aS)- 2-fluorotetrahydro-1H- pyrrolizin-7a(5H)-yl)meth- oxy)quinazolin-7-yl)-7- fluorobenzo[d]thiazol-2- amine | 602.4 [M − H]_ |
| 622 | 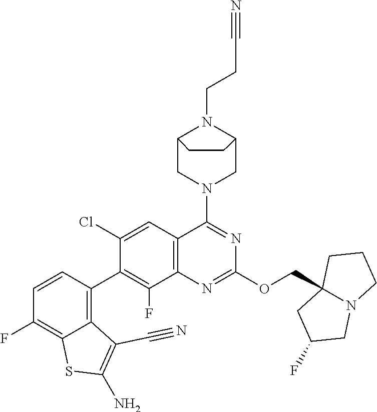 [SMILES: N#CCCN1C2CCC1CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4- ((1R,5S)-8-(2-cyanoethyl)- 3,8-diazabicyclo[3.2.1]- octan-3-yl)-8-fluoro-2- (((2R,7aS)-2-fluorotetra- hydro-1H-pyrrolizin-7a(5H)- yl)methoxy)quinazolin-7- yl)-7-fluorobenzo[b]thio- phene-3-carbonitrile | 693.7 |
| 623 | 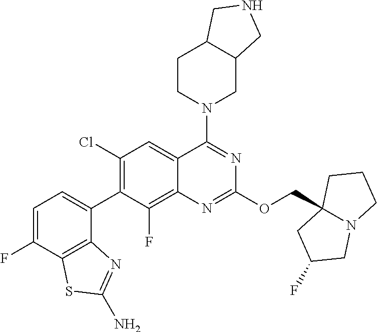 [SMILES: Nc1nc2c(-c3c(Cl)cc4c(N5CCC6CNCC6C5)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin-7a(5H)- yl)methoxy)-4-(octahydro- 5H-pyrrolo[3,4-c]pyridin-5- yl)quinazolin-7-yl)-7-fluoro- benzo[d]thiazol-2-amine | 630.1 |
| 624 | 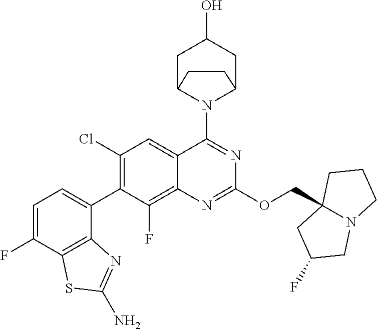 [SMILES: Nc1nc2c(-c3c(Cl)cc4c(N5C6CCC5CC(O)C6)nc(OC[C@@]56CCCN5C[C@H](F)C6)nc4c3F)ccc(F)c2s1] | (1R,5S)-8-(7-(2-amino-7- fluorobenzo[d]thiazol-4-yl)- 6-chloro-8-fluoro-2- (((2R,7aS)-2-fluorotetra- hydro-1H-pyrrolizin-7a(5H)- yl)methoxy)quinazolin-4- yl)-8-azabicyclo[3.2.1]- octan-3-ol | 631.4 |
| 625 | 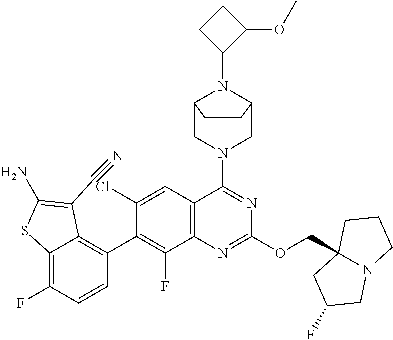 [SMILES: COC1CCC1N1C2CCC1CN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8- fluoro-2-(((2R,7aS)-2- fluorotetrahydro-1H-pyrrol- izin-7a(5H)-yl)methoxy)-4- ((1R,5S)-8-(2-methoxy- cyclobutyl)-3,8-diazabicyclo- [3.2.1]octan-3-yl)quinazolin- 7-yl)-7-fluorobenzo[b]thio- phene-3-carbonitrile | 724.4 |
| 626 | 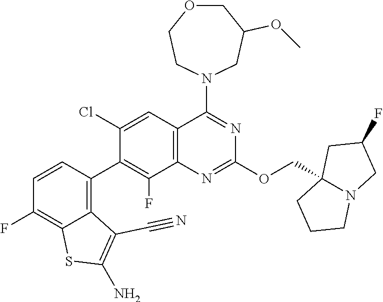 [SMILES: COC1COCCN(c2nc(OC[C@@]34CCCN3C[C@H](F)C4)nc3c(F)c(-c4ccc(F)c5sc(N)c(C#N)c45)c(Cl)cc23)C1] | 2-amino-4-(6-chloro-8- fluoro-2-(((2R,7aS)-2- fluorotetrahydro-1H-pyrrol- izin-7a(5H)-yl)methoxy)-4- (6-methoxy-1,4-oxazepan- 4-yl)quinazolin-7-yl)-7- fluorobenzo[b]thiophene- 3-carbonitrile | 659.2 |
| 627 | 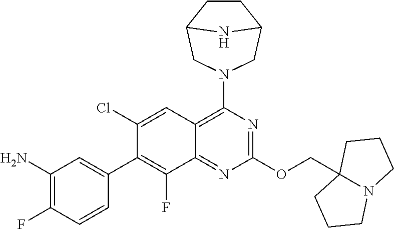 [SMILES: Nc1cc(-c2c(Cl)cc3c(N4CC5CCC(C4)N5)nc(OCC45CCCN4CCC5)nc3c2F)ccc1F] | 5-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-2- fluoroaniline | 541.3 |
| 628 | 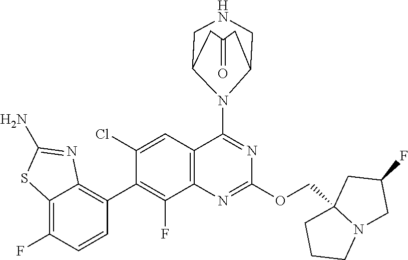 [SMILES: Nc1nc2c(-c3c(Cl)cc4c(N5C6CNCC5CC(=O)C6)nc(OC[C@@]56CCCN5C[C@H](F)C6)nc4c3F)ccc(F)c2s1] | 9-(7-(2-amino-7-fluoroben- zo[d]thiazol-4-yl)-6-chloro- 8-fluoro-2-(((2R,7aS)-2- fluorotetrahydro-1H-pyrrol- izin-7a(5H)-yl)methoxy)- quinazolin-4-yl)-3,9-diaza- bicyclo[3.3.1]nonan-7-one | 644.2 |
| 629 | 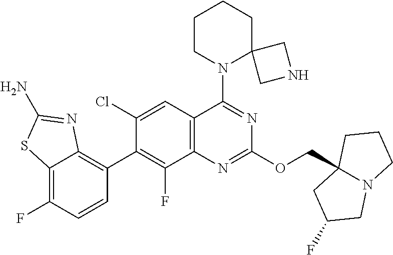 [SMILES: Nc1nc2c(-c3c(Cl)cc4c(N5CCCCC56CNC6)nc(OC[C@@]56CCCN5C[C@H](F)C6)nc4c3F)ccc(F)c2s1] | 4-(6-chloro-8-fluoro-2- (((2R,7aS)-2-fluorotetra- hydro-1H-pyrrolizin-7a(5H)- yl)methoxy)-4-(2,5-diaza- spiro[3.5]nonan-5-yl)quin- azolin-7-yl)-7-fluorobenzo- [d]thiazol-2-amine | 630.2 |
| 630 | 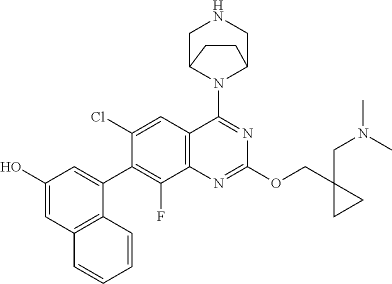 [SMILES: CN(C)CC1(COc2nc(N3C4CCC3CNC4)c3cc(Cl)c(-c4cc(O)cc5ccccc45)c(F)c3n2)CC1] | 4-(4-((1R,5S)-3,8-diaza- bicyclo[3.2.1]octan-8-yl)- 6-chloro-2-((1-((dimethyl- amino)methyl)cycloprop- yl)methoxy)-8-fluoroquin- azolin-7-yl)naphthalen-2- ol | 562.4 |
| 631 | 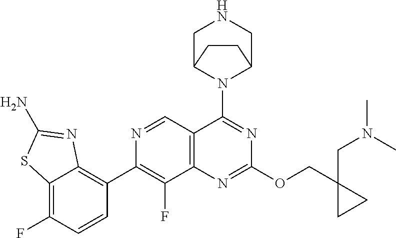 [SMILES: CN(C)CC1(COc2nc(N3C4CCC3CNC4)c3cnc(-c4ccc(F)c5sc(N)nc45)c(F)c3n2)CC1] | 4-(4-((1R,5S)-3,8-diaza- bicyclo[3.2.1]octan-8-yl)- 2-((1-(dimethylamino)- methyl)cyclopropyl)meth- oxy)-8-fluoropyrido[4,3-d]- pyrimidin-7-yl)-7-fluoro- benzo[d]thiazol-2-amine | 553.4 |
| 632 | 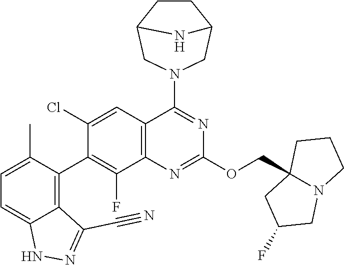 [SMILES: Cc1ccc2[nH]nc(C#N)c2c1-c1c(Cl)cc2c(N3CC4CCC(C3)N4)nc(OC[C@@]34CCCN3C[C@H](F)C4)nc2c1F] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quin-azolin-7-yl)-5-methyl- 1H-indazole-3-carbonitrile | 603.4 [M − H]− |
| 633 | 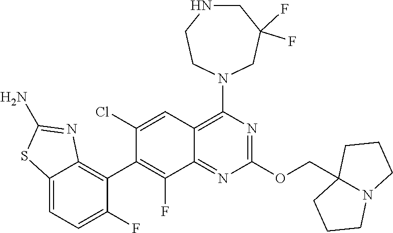 [SMILES: Nc1nc2c(-c3c(Cl)cc4c(N5CCNCC(F)(F)C5)nc(OCC56CCCN5CCC6)nc4c3F)c(F)ccc2s1] | 4-(6-chloro-4-(6,6-difluoro- 1,4-diazepan-1-yl)-8-fluoro- 2-((tetrahydro-1H-pyrrol- izin-7a(5H)-yl)methoxy)- quinazolin-7-yl)-5-fluoro- benzo[d]thiazol-2-amine | 622.2 |
| 634 | 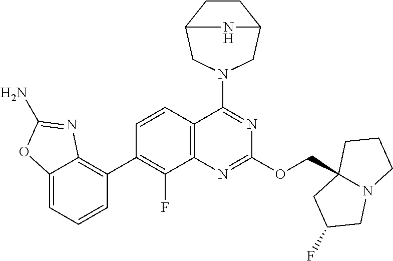 [SMILES: Nc1nc2c(-c3ccc4c(N5CC6CCC(C5)N6)nc(OC[C@@]56CCCN5C[C@H](F)C6)nc4c3F)cccc2o1] | 4-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)benzo[d]oxazol-2-amine | 548.4 |
| 635 | 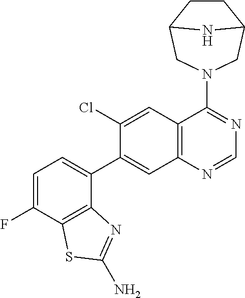 [SMILES: Nc1nc2c(-c3cc4ncnc(N5CC6CCC(C5)N6)c4cc3Cl)ccc(F)c2s1] | 4-(4-(3,8-diazabicyclo- [3.2.1]octan-3-yl)-6-chloro- quinazolin-7-yl)-7-fluoro- benzo[d]thiazol-2-amine | 441.4 |
| 636 | 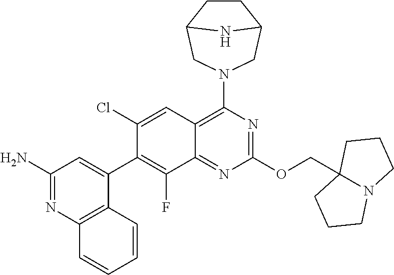 [SMILES: Nc1cc(-c2c(Cl)cc3c(N4CC5CCC(C4)N5)nc(OCC45CCCN4CCC5)nc3c2F)c2ccccc2n1] | 4-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)quin- olin-2-amine | 574.2 |
| 637 | 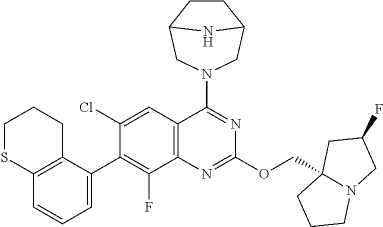 [SMILES: Fc1c(-c2cccc3c2CCCS3)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-6-chloro-8- fluoro-2-(((2R,7aS)-2-fluoro- tetrahydro-1H-pyrrolizin- 7a(5H)-yl)methoxy)-7-(thio- chroman-5-yl)quinazoline | 598.2 |
| 638 | 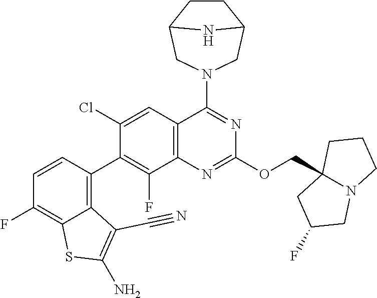 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-2-amino-7- fluorobenzo[b]thiophene-3- carbonitrile | 640.1 |
| 639 | 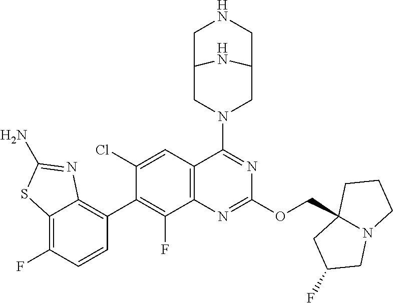 [SMILES: Nc1nc2c(-c3c(Cl)cc4c(N5CC6CNCC(C5)N6)nc(OC[C@@]56CCCN5C[C@H](F)C6)nc4c3F)ccc(F)c2s1] | 4-(4-(3,7,9-triazabicyclo- [3.3.1]nonan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 631.4 |
| 640 | 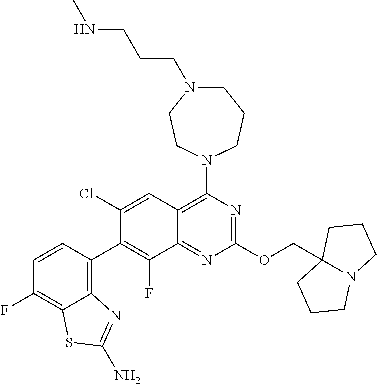 [SMILES: CNCCCN1CCCN(c2nc(OCC34CCCN3CCC4)nc3c(F)c(-c4ccc(F)c5sc(N)nc45)c(Cl)cc23)CC1] | 4-(6-chloro-8-fluoro-4-(4- (3-(methylamino)propyl)- 1,4-diazepan-1-yl)-2- ((tetrahydro-1H-pyrrolizin- 7a(5H)-yl)methoxy)quin- azolin-7-yl)-7-fluorobenzo- [d]thiazol-2-amine | 657.3 |
| 641 | 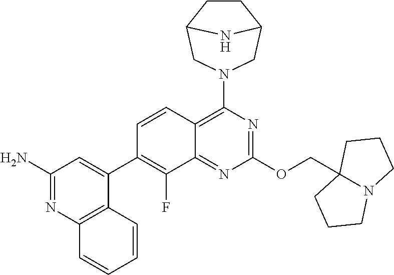 [SMILES: Nc1cc(-c2ccc3c(N4CC5CCC(C4)N5)nc(OCC45CCCN4CCC5)nc3c2F)c2ccccc2n1] | 4-(4-(3,8-diazabicyclo- [3.2.1]octan-3-yl)-8-fluoro- 2-((tetrahydro-1H-pyrrol- izin-7a(5H)-yl)methoxy)- quinazolin-7-yl)quinolin-2- amine | 540.3 |
| 642 | 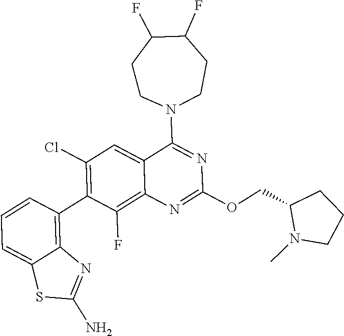 [SMILES: CN1CCC[C@H]1COc1nc(N2CCC(F)C(F)CC2)c2cc(Cl)c(-c3cccc4sc(N)nc34)c(F)c2n1] | 4-(6-chloro-4-(4,5-difluoro- azepan-1-yl)-8-fluoro-2- (((S)-1-methylpyrrolidin-2- yl)methoxy)quinazolin-7-yl)- benzo[d]thiazol-2-amine | 577.3 |
| 643 | 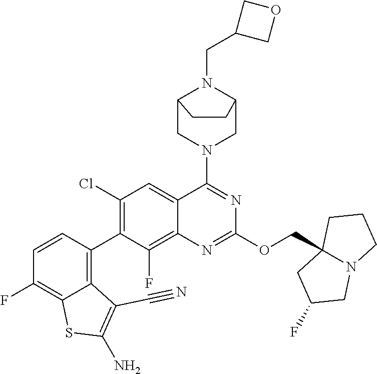 [SMILES: N#Cc1c(N)sc2c(F)ccc(-c3c(Cl)cc4c(N5CC6CCC(C5)N6CC5COC5)nc(OC[C@@]56CCCN5C[C@H](F)C6)nc4c3F)c12] | 2-amino-4-(6-chloro-8- fluoro-2-(((2R,7aS)-2- fluorotetrahydro-1H- pyrrolizin-7a(5H)-yl)- methoxy)-4-((1R,5S)-8- (oxetan-3-yl-methyl)- 3,8-diazabicyclo[3.2.1]- octan-3-yl)quinazolin- 7-yl)-7-fluorobenzo[b]- thiophene-3-carbonitrile | 710.2 |
| 644 | 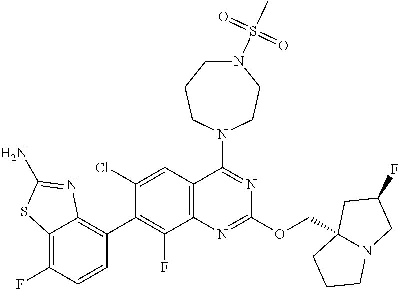 [SMILES: CS(=O)(=O)N1CCCN(c2nc(OC[C@@]34CCCN3C[C@H](F)C4)nc3c(F)c(-c4ccc(F)c5sc(N)nc45)c(Cl)cc23)CC1] | 4-(6-chloro-8-fluoro-2- (((2R,7aS)-2-fluorotetra- hydro-1H-pyrrolizin- 7a(5H)-yl)methoxy)-4-(4- (methylsulfonyl)-1,4- diazepan-1-yl)quinazolin- 7-yl)-7-fluorobenzo[d]thia- zol-2-amine | 682.5 |
| 645 | 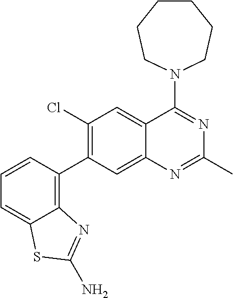 [SMILES: Cc1nc(N2CCCCCC2)c2cc(Cl)c(-c3cccc4sc(N)nc34)cc2n1] | 4-(4-(azepan-1-yl)-6-chloro- 2-methylquinazolin-7-yl)- benzo[d]thiazol-2-amine | 423.0 |
| 646 | 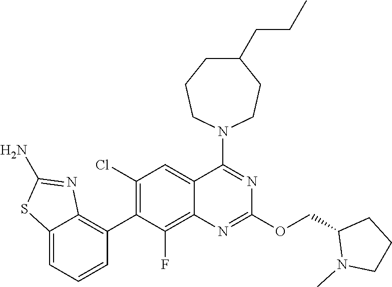 [SMILES: CCCC1CCCN(c2nc(OC[C@@H]3CCCN3C)nc3c(F)c(-c4cccc5sc(N)nc45)c(Cl)cc23)CC1] | 4-(6-chloro-8-fluoro-2-(((S)- 1-methylpyrrolidin-2-yl)- methoxy)-4-(4-propylaze- pan-1-yl)quinazolin-7-yl)- benzo[d]thiazol-2-amine | 583.2 |
| 647 | 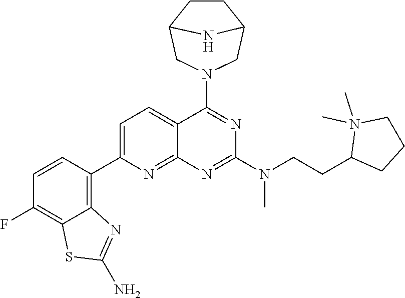 | 4-(4-(3,8-diazabicyclo- [3.2.1]octan-3-yl)-2-((2- (1,1-dimethyl-114-pyrrol- idin-2-yl)ethyl)(methyl)- amino)pyrido[2,3-d]pyr- imidin-7-yl)-7-fluoroben- zo[d]thiazol-2-amine | 563.0 |
| 648 | 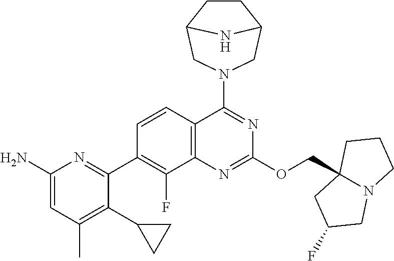 [SMILES: Cc1cc(N)nc(-c2ccc3c(N4CC5CCC(C4)N5)nc(OC[C@@]45CCCN4C[C@H](F)C5)nc3c2F)c1C1CC1] | 6-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-5-cyclopropyl-4-methyl- pyridin-2-amine | 562.5 |
| 649 | 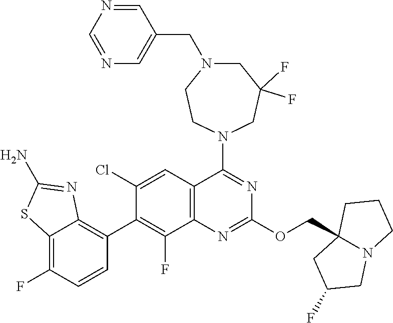 [SMILES: Nc1nc2c(-c3c(Cl)cc4c(N5CCN(Cc6cncnc6)CC(F)(F)C5)nc(OC[C@@]56CCCN5C[C@H](F)C6)nc4c3F)ccc(F)c2s1] | 4-(6-chloro-4-(6,6-difluoro- 4-(pyrimidin-5-ylmethyl)- 1,4-diazepan-1-yl)-8-fluoro- 2-(((2R,7aS)-2-fluorotetra- hydro-1H-pyrrolizin-7a(5H)- yl)methoxy)quinazolin-7- yl)-7-fluorobenzo[d]thiazol- 2-amine | 730.5 [M − H]− |
| 650 | 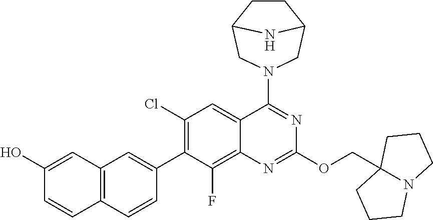 [SMILES: Oc1ccc2ccc(-c3c(Cl)cc4c(N5CC6CCC(C5)N6)nc(OCC56CCCN5CCC6)nc4c3F)cc2c1] | 7-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)naph- thalen-2-ol | 574.2 |
| 651 | 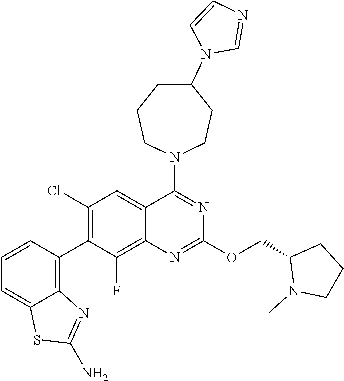 [SMILES: CN1CCC[C@H]1COc1nc(N2CCCC(n3ccnc3)CC2)c2cc(Cl)c(-c3cccc4sc(N)nc34)c(F)c2n1] | 4-(4-(4-(1H-imidazol-1-yl)- azepan-1-yl)-6-chloro-8- fluoro-2-(((S)-1-methyl- pyrrolidin-2-yl)methoxy)- quinazolin-7-yl)benzo[d]- thiazol-2-amine | 607.3 |
| 652 | 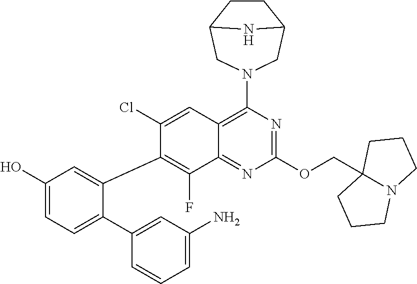 [SMILES: Nc1cccc(-c2ccc(O)cc2-c2c(Cl)cc3c(N4CC5CCC(C4)N5)nc(OCC45CCCN4CCC5)nc3c2F)c1] | 2-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-3′- amino-[1,1′-biphenyl]-4-ol | 615.3 |
| 653 | 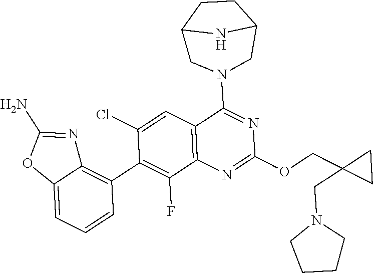 [SMILES: Nc1nc2c(-c3c(Cl)cc4c(N5CC6CCC(C5)N6)nc(OCC5(CN6CCCC6)CC5)nc4c3F)cccc2o1] | 4-(4-(3,8-diazabicyclo- [3.2.1]octan-3-yl)-6-chloro- 8-fluoro-2-((1-(pyrrolidin-1- ylmethyl)cyclopropyl)meth- oxy)quinazolin-7-yl)benzo- [d]oxazol-2-amine | 578.5 |
| 654 | 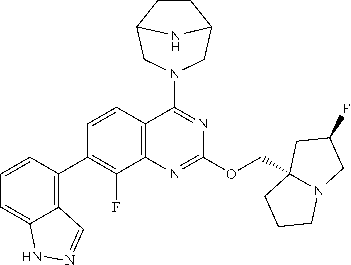 [SMILES: Fc1c(-c2cccc3[nH]ncc23)ccc2c(N3CC4CCC(C3)N4)nc(OC[C@@]34CCCN3C[C@H](F)C4)nc12] | 4-(3,8-diazabicyclo[3.2.1]- octan-3-yl)-8-fluoro-2- (((2R,7aS)-2-fluorotetra- hydro-1H-pyrrolizin-7a(5H)- yl)methoxy)-7-(1H-indazol- 4-yl)quinazoline | 532.5 |
| 655 | 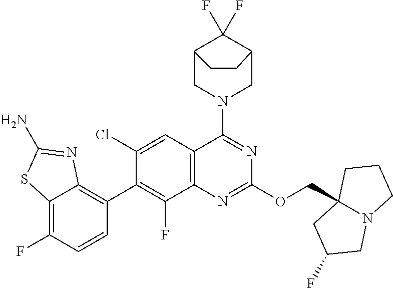 [SMILES: Nc1nc2c(-c3c(Cl)cc4c(N5CC6CCC(C5)C6(F)F)nc(OC[C@@]56CCCN5C[C@H](F)C6)nc4c3F)ccc(F)c2s1] | 4-(6-chloro-4-((1R,5S)-8,8- difluoro-3-azabicyclo[3.2.1]- octan-3-yl)-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 651.3 |
| 656 | 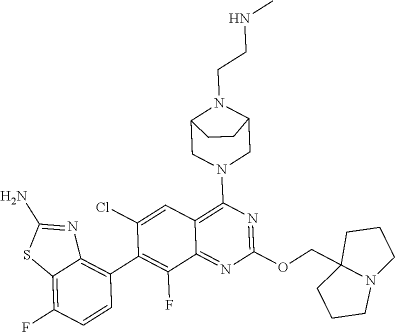 [SMILES: CNCCN1C2CCC1CN(c1nc(OCC34CCCN3CCC4)nc3c(F)c(-c4ccc(F)c5sc(N)nc45)c(Cl)cc13)C2] | 4-(6-chloro-8-fluoro-4- ((1R,5S)-8-(2-(methyl- amino)-ethyl)-3,8-diaza- bicyclo[3.2.1]octan-3-yl)- 2-((tetrahydro-1H-pyrrol- izin-7a(5H)-yl)methoxy)- quinazolin-7-yl)-7-fluoro- benzo[d]thiazol-2-amine | 655.2 |
| 657 | 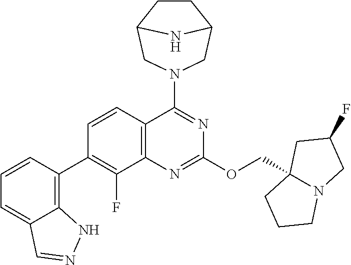 [SMILES: Fc1c(-c2cccc3cn[nH]c23)ccc2c(N3CC4CCC(C3)N4)nc(OC[C@@]34CCCN3C[C@H](F)C4)nc12] | 4-(3,8-diazabicyclo[3.2.1]- octan-3-yl)-8-fluoro-2- (((2R,7aS)-2-fluorotetra- hydro-1H-pyrrolizin-7a(5H)- yl)methoxy)-7-(1H-indazol- 7-yl)quinazoline | 532.5 |
| 658 | 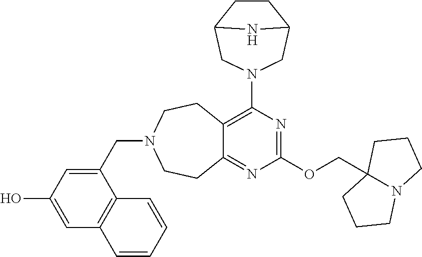 | 4-((4-(3,8-diazabicyclo- [3.2.1]-octan-3-yl)-2- ((tetrahydro-1H-pyrrolizin- 7a(5H)-yl)methoxy)-5,6,8,9- tetrahydro-7H-pyrimido- [4,5-d]azepin-7-yl)methyl)- naphthalen-2-ol | 555.5 |
| 659 | 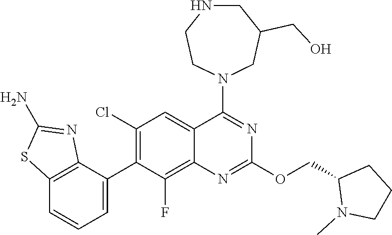 [SMILES: CN1CCC[C@H]1COc1nc(N2CCNCC(CO)C2)c2cc(Cl)c(-c3cccc4sc(N)nc34)c(F)c2n1] | (1-(7-(2-aminobenzo[d]- thiazol-4-yl)-6-chloro-8- fluoro-2-(((S)-1-methylpyr- rolidin-2-yl)methoxy)quin- azolin-4-yl)-1,4-diazepan- 6-yl)methanol | 572.8 |
| 660 | 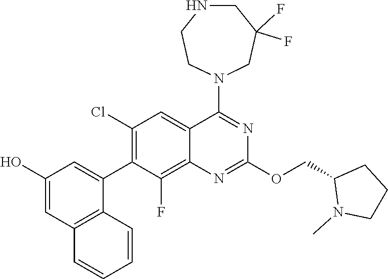 [SMILES: CN1CCC[C@H]1COc1nc(N2CCNCC(F)(F)C2)c2cc(Cl)c(-c3cc(O)cc4ccccc34)c(F)c2n1] | 4-(6-chloro-4-(6,6-difluoro- 1,4-diazepan-1-yl)-8-fluoro- 2-(((S)-1-methylpyrrolidin- 2-yl)methoxy)quinazolin- 7-yl)naphthalen-2-ol | 572.7 |
| 661 | 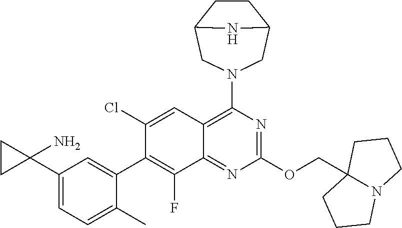 [SMILES: Cc1ccc(C2(N)CC2)cc1-c1c(Cl)cc2c(N3CC4CCC(C3)N4)nc(OCC34CCCN3CCC4)nc2c1F] | 1-(3-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-4- methylphenyl)cyclopropan- 1-amine | 577.2 |
| 662 | 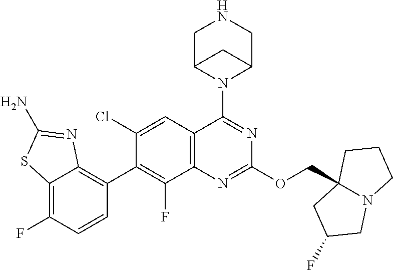 [SMILES: Nc1nc2c(-c3c(Cl)cc4c(N5C6CNCC5C6)nc(OC[C@@]56CCCN5C[C@H](F)C6)nc4c3F)ccc(F)c2s1] | 4-(4-(3,6-diazabicyclo- [3.1.1]heptan-6-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 602.0 |
| 663 | 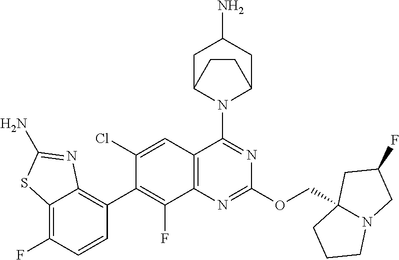 [SMILES: Nc1nc2c(-c3c(Cl)cc4c(N5C6CCC5CC(N)C6)nc(OC[C@@]56CCCN5C[C@H](F)C6)nc4c3F)ccc(F)c2s1] | 4-(4-((1R,5S)-3-amino-8- azabicyclo[3.2.1]octan-8- yl)-6-chloro-8-fluoro-2- (((2R,7aS)-2-fluorotetra- hydro-1H-pyrrolizin-7a(5H)- yl)methoxy)quinazolin-7- yl)-7-fluorobenzo[d]thiazol- 2-amine | 631.2 |
| 664 | 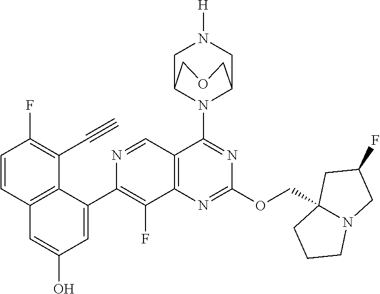 [SMILES: C#Cc1c(F)ccc2cc(O)cc(-c3ncc4c(N5C6CNCC5COC6)nc(OC[C@@]56CCCN5C[C@H](F)C6)nc4c3F)c12] | 4-(4-(3-oxa-7,9-diaza- bicyclo[3.3.1]nonan-9-yl)- 8-fluoro-2-(((2R,7aS)-2- fluorotetrahydro-1H-pyrrol- izin-7a(5H)-yl)methoxy)- pyrido[4,3-d]pyrimidin-7- yl)-5-ethynyl-6-fluoronaph- thalen-2-ol | 617.3 |
| 665 | 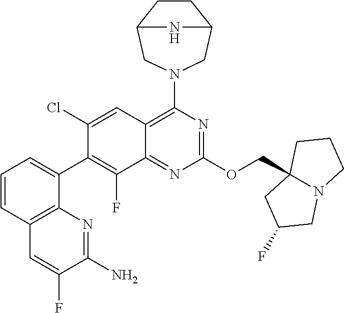 [SMILES: Nc1nc2c(-c3c(Cl)cc4c(N5CC6CCC(C5)N6)nc(OC[C@@]56CCCN5C[C@H](F)C6)nc4c3F)cccc2cc1F] | 8-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-3-fluoro- quinolin-2-amine | 610.2 |
| 666 | 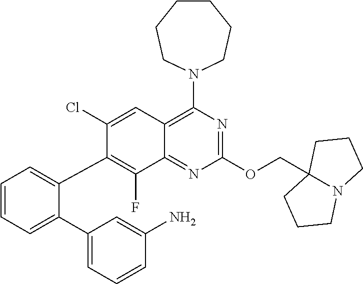 [SMILES: Nc1cccc(-c2ccccc2-c2c(Cl)cc3c(N4CCCCCC4)nc(OCC45CCCN4CCC5)nc3c2F)c1] | 2′-(4-(azepan-1-yl)-6-chloro- 8-fluoro-2-((tetrahydro-1H- pyrrolizin-7a(5H)-yl)meth- oxy)quinazolin-7-yl)-[1,1′- biphenyl]-3-amine | 586.3 |
| 667 | 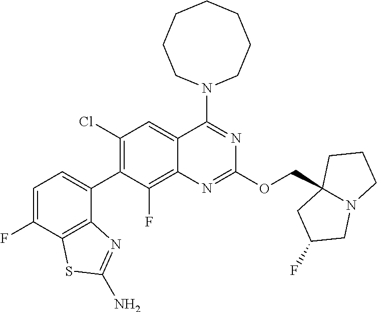 [SMILES: Nc1nc2c(-c3c(Cl)cc4c(N5CCCCCCC5)nc(OC[C@@]56CCCN5C[C@H](F)C6)nc4c3F)ccc(F)c2s1] | 4-(4-(azocan-1-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 617.5 |
| 668 | 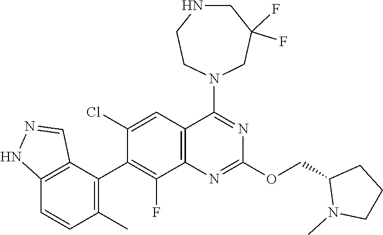 [SMILES: Cc1ccc2[nH]ncc2c1-c1c(Cl)cc2c(N3CCNCC(F)(F)C3)nc(OC[C@@H]3CCCN3C)nc2c1F] | 6-chloro-4-(6,6-difluoro-1,4- diazepan-1-yl)-8-fluoro-7-(5- methyl-1H-indazol-4-yl)-2- (((S)-1-methylpyrrolidin-2- yl)methoxy)quinazoline | 560.2 |
| 669 | 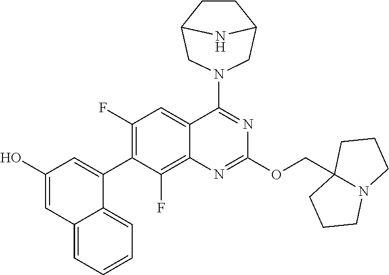 [SMILES: Oc1cc(-c2c(F)cc3c(N4CC5CCC(C4)N5)nc(OCC45CCCN4CCC5)nc3c2F)c2ccccc2c1] | 4-(4-(3,8-diazabicyclo- [3.2.1]octan-3-yl)-6,8- difluoro-2-((tetrahydro- 1H-pyrrolizin-7a(5H)- yl)methoxy)quinazolin- 7-yl)naphthalen-2-ol | 558.3 |
| 670 | 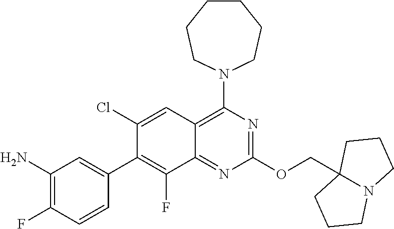 [SMILES: Nc1cc(-c2c(Cl)cc3c(N4CCCCCC4)nc(OCC45CCCN4CCC5)nc3c2F)ccc1F] | 5-(4-(azepan-1-yl)-6-chloro- 8-fluoro-2-((tetrahydro-1H- pyrrolizin-7a(5H)-yl)meth- oxy)quinazolin-7-yl)-2- fluoroaniline | 528.2 |
| 671 | 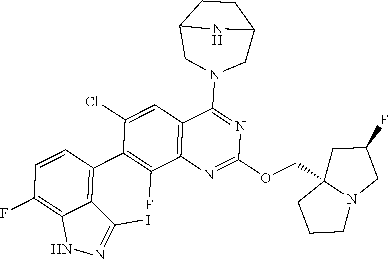 [SMILES: Fc1c(-c2ccc(F)c3[nH]nc(I)c23)c(Cl)cc2c(N3CC4CCC(C3)N4)nc(OC[C@@]34CCCN3C[C@H](F)C4)nc12] | 4-(3,8-diazabicyclo[3.2.1]- octan-3-yl)-6-chloro-8- fluoro-7-(7-fluoro-3-iodo- 1H-indazol-4-yl)-2- (((2R,7aS)-2-fluorotetra- hydro-1H-pyrrolizin-7a(5H)- yl)methoxy)quinazoline | 710.3 |
| 672 | 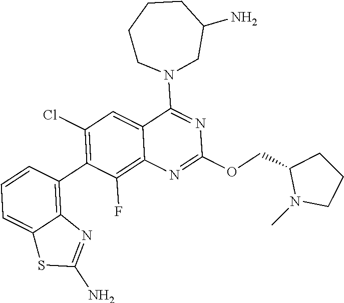 [SMILES: CN1CCC[C@H]1COc1nc(N2CCCCC(N)C2)c2cc(Cl)c(-c3cccc4sc(N)nc34)c(F)c2n1] | 4-(4-(3-aminoazepan-1-yl)- 6-chloro-8-fluoro-2-(((S)-1- methylpyrrolidin-2-yl)meth- oxy)quinazolin-7-yl)ben- zo[d]thiazol-2-amine | 556.2 |
| 673 | 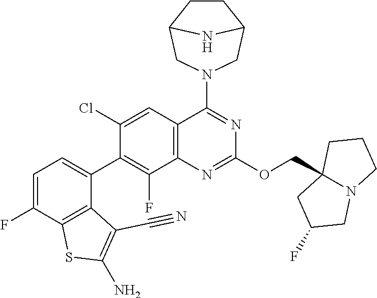 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-2-amino- 7-fluorobenzo[b]thiophene- 3-carbonitrile | 640.1 |
| 674 | 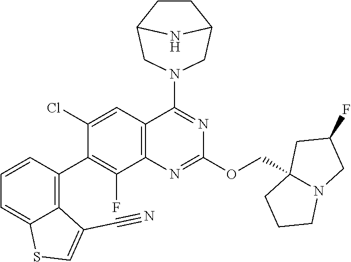 [SMILES: N#Cc1csc2cccc(-c3c(Cl)cc4c(N5CC6CCC(C5)N6)nc(OC[C@@]56CCCN5C[C@H](F)C6)nc4c3F)c12] | 4-(4-(3,8-diazabicyclo- [3.2.1]-octan-3-yl)-6-chloro- 8-fluoro-2-(((2R,7aS)-2- fluorotetrahydro-1H-pyrrol- izin-7a(5H)-yl)methoxy)- quinazolin-7-yl)benzo[b]- thiophene-3-carbonitrile | 607.2 |
| 675 | 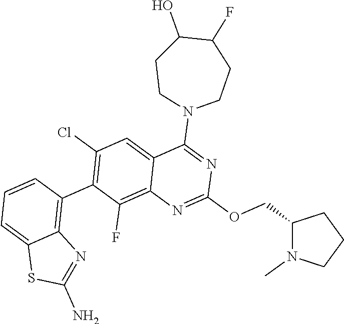 [SMILES: CN1CCC[C@H]1COc1nc(N2CCC(O)C(F)CC2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)-5-fluoro- azepan-4-ol | 557.2 |
| 676 | 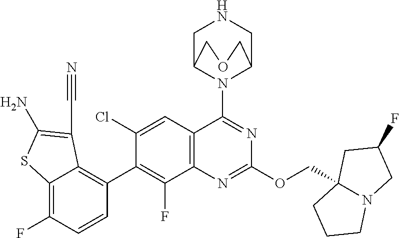 [SMILES: N#Cc1c(N)sc2c(F)ccc(-c3c(Cl)cc4c(N5C6CNCC5COC6)nc(OC[C@@]56CCCN5C[C@H](F)C6)nc4c3F)c12] | 4-(4-(3-oxa-7,9-diaza- bicyclo[3.3.1]nonan-9-yl)- 6-chloro-8-fluoro-2- (((2R,7aS)-2-fluorotetra- hydro-1H-pyrrolizin-7a(5H)- yl)methoxy)quinazolin-7- yl)-2-amino-7-fluorobenzo- [b]thiophene-3-carbonitrile | 656.4 |
| 677 | 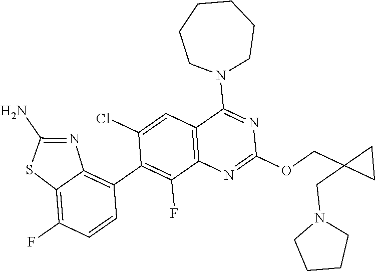 [SMILES: Nc1nc2c(-c3c(Cl)cc4c(N5CCCCCC5)nc(OCC5(CN6CCCC6)CC5)nc4c3F)ccc(F)c2s1] | 4-(4-(azepan-1-yl)-6-chloro- 8-fluoro-2-((1-(pyrrolidin-1- ylmethyl)cyclopropyl)meth- oxy)quinazolin-7-yl)-7- fluorobenzo[d]thiazol-2- amine | 599.5 |
| 678 | 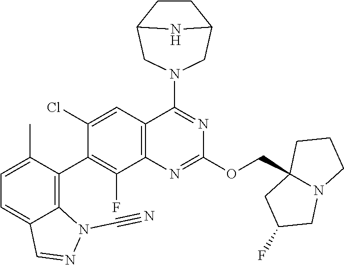 [SMILES: Cc1ccc2cnn(C#N)c2c1-c1c(Cl)cc2c(N3CC4CCC(C3)N4)nc(OC[C@@]34CCCN3C[C@H](F)C4)nc2c1F] | 7-(4-(3,8-diazabicyclo- [3.2.1]octan-3-yl)-6-chloro- 8-fluoro-2-(((2R,7aS)-2- fluorotetrahydro-1H-pyrrol- izin-7a(5H)-yl)methoxy)- quinazolin-7-yl)-6-methyl- 1H-indazole-1-carbonitrile | 605.4 |
| 679 | 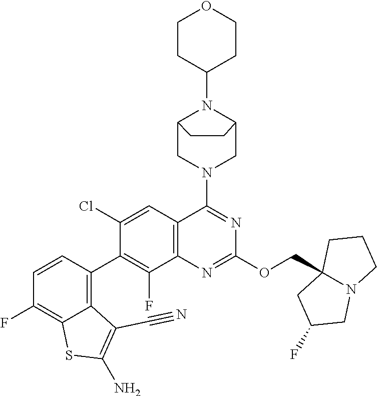 [SMILES: N#Cc1c(N)sc2c(F)ccc(-c3c(Cl)cc4c(N5CC6CCC(C5)N6C5CCOCC5)nc(OC[C@@]56CCCN5C[C@H](F)C6)nc4c3F)c12] | 2-amino-4-(6-chloro-8- fluoro-2-(((2R,7aS)-2- fluorotetrahydro-1H-pyrrol- izin-7a(5H)-yl)methoxy)- 4-((1R,5S)-8-(tetrahydro- 2H-pyran-4-yl)-3,8-diaza- bicyclo[3.2.1]octan-3-yl)- quinazolin-7-yl)-7-fluoro- benzo[b]thiophene-3- carbonitrile | 724.1 |
| 680 | 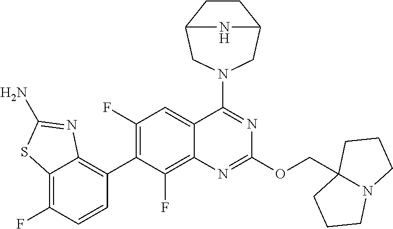 [SMILES: Nc1nc2c(-c3c(F)cc4c(N5CC6CCC(C5)N6)nc(OCC56CCCN5CCC6)nc4c3F)ccc(F)c2s1] | 4-(4-(3,8-diazabicyclo- [3.2.1]octan-3-yl)-6,8- difluoro-2-((tetrahydro- 1H-pyrrolizin-7a(5H)- yl)methoxy)quinazolin-7- yl)-7-fluorobenzo[d]thiazol- 2-amine | 582.3 |
| 681 | 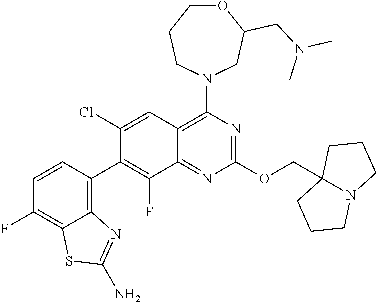 [SMILES: CN(C)CC1CN(c2nc(OCC34CCCN3CCC4)nc3c(F)c(-c4ccc(F)c5sc(N)nc45)c(Cl)cc23)CCCO1] | 4-(6-chloro-4-(2-((dimethyl- amino)methyl)-1,4-oxaze- pan-4-yl)-8-fluoro-2-((tetra- hydro-1H-pyrrolizin-7a(5H)- yl)methoxy)quinazolin-7- yl)-7-fluorobenzo[d]thiazol- 2-amine | 644.2 |
| 682 | 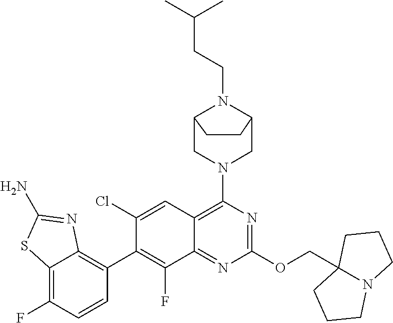 [SMILES: CC(C)CCN1C2CCC1CN(c1nc(OCC34CCCN3CCC4)nc3c(F)c(-c4ccc(F)c5sc(N)nc45)c(Cl)cc13)C2] | 4-(6-chloro-8-fluoro-4- ((1R,5S)-8-isopentyl-3,8- diazabicyclo[3.2.1]octan-3- yl)-2-((tetrahydro-1H-pyr- rolizin-7a(5H)-yl)methoxy)- quinazolin-7-yl)-7-fluoro- benzo[d]thiazol-2-amine | 668.2 |
| 683 | 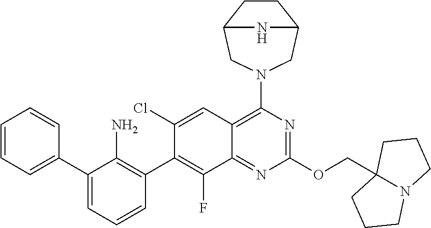 [SMILES: Nc1c(-c2ccccc2)cccc1-c1c(Cl)cc2c(N3CC4CCC(C3)N4)nc(OCC34CCCN3CCC4)nc2c1F] | 3-(4-(3,8-diazabicyclo- [3.2.1]-octan-3-yl)-6-chloro- 8-fluoro-2-((tetrahydro-1H- pyrrolizin-7a(5H)-yl)meth- oxy)quinazolin-7-yl)-[1,1′- biphenyl]-2-amine | 599.4 |
| 684 | 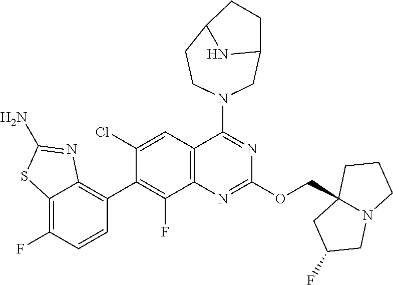 [SMILES: Nc1nc2c(-c3c(Cl)cc4c(N5CCC6CCC(C5)N6)nc(OC[C@@]56CCCN5C[C@H](F)C6)nc4c3F)ccc(F)c2s1] | 4-(4-(3,9-diazabicyclo- [4.2.1]nonan-3-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 631.2 |
| 685 | 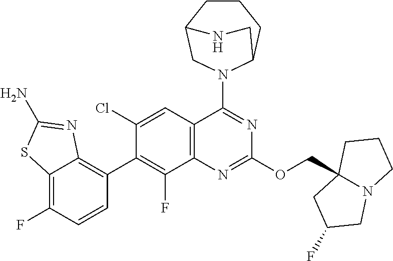 [SMILES: Nc1nc2c(-c3c(Cl)cc4c(N5CC6CCCC5CN6)nc(OC[C@@]56CCCN5C[C@H](F)C6)nc4c3F)ccc(F)c2s1] | 4-(4-(6,8-diazabicyclo- [3.2.2]nonan-6-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 631.4 |
| 686 | 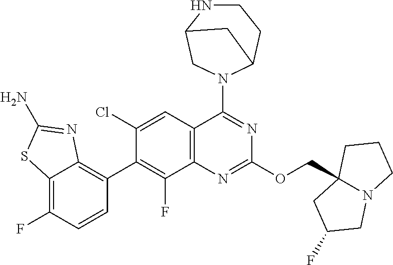 [SMILES: Nc1nc2c(-c3c(Cl)cc4c(N5CC6CC5CCN6)nc(OC[C@@]56CCCN5C[C@H](F)C6)nc4c3F)ccc(F)c2s1] | 4-(4-(2,6-diazabicyclo- [3.2.1]octan-6-yl)-6-chloro- 8-fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 617.2 |
| 687 | 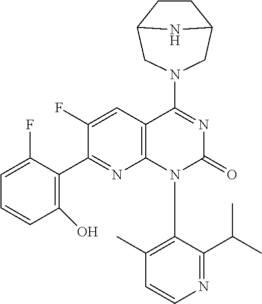 [SMILES: Cc1ccnc(C(C)C)c1-n1c(=O)nc(N2CC3CCC(C2)N3)c2cc(F)c(-c3c(O)cccc3F)nc21] | 4-(3,8-diazabicyclo[3.2.1]- octan-3-yl)-6-fluoro-7-(2- fluoro-6-hydroxyphenyl)-1- (2-isopropyl-4-methylpyr- idin-3-yl)pyrido[2,3-d]pyr- imidin-2(1H)-one | 519.4 |
| 688 | 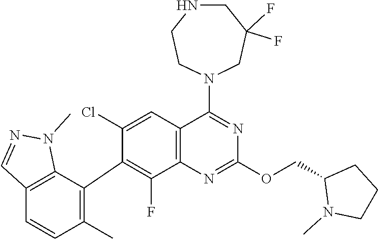 [SMILES: Cc1ccc2cnn(C)c2c1-c1c(Cl)cc2c(N3CCNCC(F)(F)C3)nc(OC[C@@H]3CCCN3C)nc2c1F] | 6-chloro-4-(6,6-difluoro- 1,4-diazepan-1-yl)-7-(1,6- dimethyl-1H-indazol-7-yl)- 8-fluoro-2-(((S)-1-methyl- pyrrolidin-2-yl)methoxy)- quinazoline | 574.6 |
| 689 | 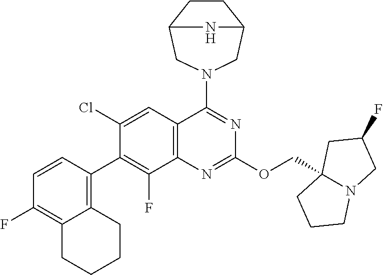 [SMILES: Fc1ccc(-c2c(Cl)cc3c(N4CC5CCC(C4)N5)nc(OC[C@@]45CCCN4C[C@H](F)C5)nc3c2F)c2c1CCCC2] | 4-(3,8-diazabicyclo[3.2.1]- octan-3-yl)-6-chloro-8- fluoro-7-(4-fluoro-5,6,7,8- tetrahydronaphthalen-1-yl)- 2-(((2R,7aS)-2-fluorotetra- hydro-1H-pyrrolizin-7a(5H)- yl)methoxy)quinazoline | 598.3 |
| 690 | 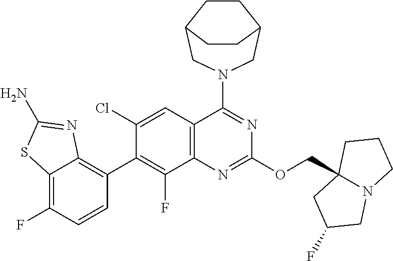 [SMILES: Nc1nc2c(-c3c(Cl)cc4c(N5CC6CCC(CC6)C5)nc(OC[C@@]56CCCN5C[C@H](F)C6)nc4c3F)ccc(F)c2s1] | 4-(4-(3-azabicyclo[3.2.2]- nonan-3-yl)-6-chloro-8- fluoro-2-(((2R,7aS)-2- fluorotetrahydro-1H-pyrrol- izin-7a(5H)-yl)methoxy)- quinazolin-7-yl)-7-fluoro- benzo[d]thiazol-2-amine | 630.2 |
| 691 | 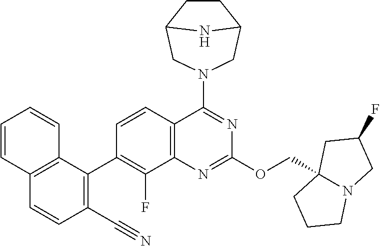 [SMILES: N#Cc1ccc2ccccc2c1-c1ccc2c(N3CC4CCC(C3)N4)nc(OC[C@@]34CCCN3C[C@H](F)C4)nc2c1F] | 1-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-2-naphthonitrile | 567.4 |
| 692 | 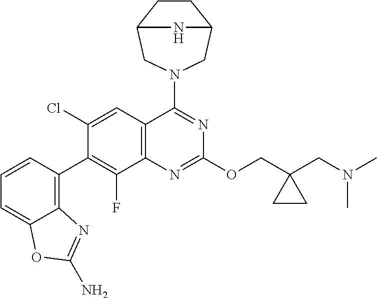 [SMILES: CN(C)CC1(COc2nc(N3CC4CCC(C3)N4)c3cc(Cl)c(-c4cccc5oc(N)nc45)c(F)c3n2)CC1] | 4-(4-(3,8-diazabicyclo- [3.2.1]-octan-3-yl)-6-chloro- 2-((1-((dimethylamino)- methyl)cyclopropyl)meth- oxy)-8-fluoroquinazolin-7- yl)benzo[d]oxazol-2-amine | 552.5 |
| 693 | 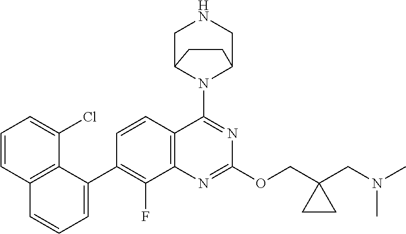 [SMILES: CN(C)CC1(COc2nc(N3C4CCC3CNC4)c3ccc(-c4cccc5cccc(Cl)c45)c(F)c3n2)CC1] | 1-(1-(((4-((1R,5S)-3,8-diaza- bicyclo[3.2.1]octan-8-yl)-7- (8-chloronaphthalen-1-yl)- 8-fluoroquinazolin-2-yl)- oxy)methyl)cyclopropyl)- N,N-dimethylmethanamine | 546.4 |
| 694 | 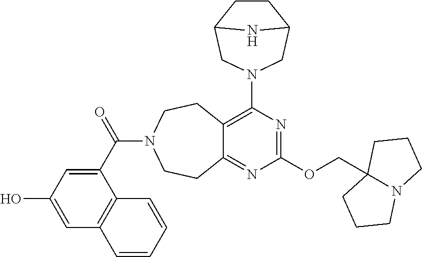 [SMILES: O=C(c1cc(O)cc2ccccc12)N1CCc2nc(OCC34CCCN3CCC4)nc(N3CC4CCC(C3)N4)c2CC1] | (4-(3,8-diazabicyclo[3.2.1]- octan-3-yl)-2-((tetrahydro- 1H-pyrrolizin-7a(5H)-yl)- methoxy)-5,6,8,9-tetrahydro- 7H-pyrimido[4,5-d]azepin- 7-yl)(3-hydroxynaphthalen- 1-yl)methanone | 569.3 |
| 695 | 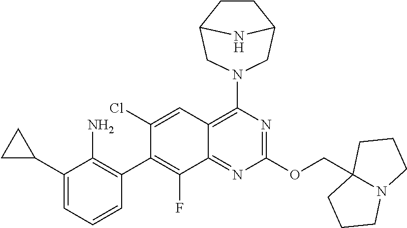 [SMILES: Nc1c(-c2c(Cl)cc3c(N4CC5CCC(C4)N5)nc(OCC45CCCN4CCC5)nc3c2F)cccc1C1CC1] | 2-(4-(3,8-diazabicyclo- [3.2.1]octan-3-yl)-6-chloro- 8-fluoro-2-((tetrahydro-1H- pyrrolizin-7a(5H)-yl)meth- oxy)quinazolin-7-yl)-6- cyclopropylaniline | 563.4 |
| 696 | 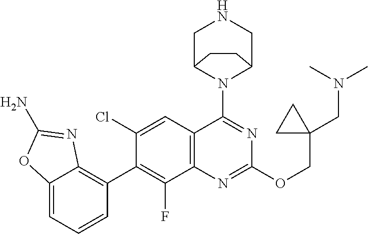 [SMILES: CN(C)CC1(COc2nc(N3C4CCC3CNC4)c3cc(Cl)c(-c4cccc5oc(N)nc45)c(F)c3n2)CC1] | 4-(4-((1R,5S)-3,8-diaza- bicyclo[3.2.1]octan-8-yl)-6- chloro-2-((1-(dimethyl- amino)methyl)cyclopropyl)- methoxy)-8-fluoroquin- azolin-7-yl)benzo[d]oxazol- 2-amine | 552.4 |
| 697 | 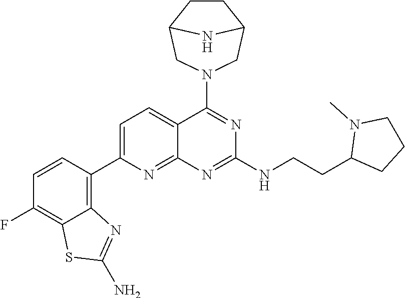 [SMILES: CN1CCCC1CCNc1nc(N2CC3CCC(C2)N3)c2ccc(-c3ccc(F)c4sc(N)nc34)nc2n1] | 4-(4-(3,8-diazabicyclo- [3.2.1]octan-3-yl)-2-((2-(1- methylpyrrolidin-2-yl)- ethyl)amino)pyrido[2,3-d]- pyrimidin-7-yl)-7-fluoro- benzo[d]thiazol-2-amine | 534 |
| 698 | 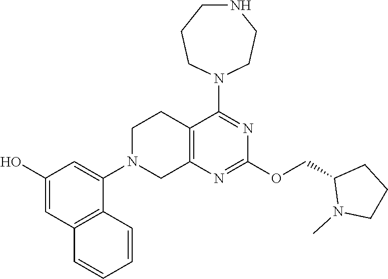 [SMILES: CN1CCC[C@H]1COc1nc2c(c(N3CCCNCC3)n1)CCN(c1cc(O)cc3ccccc13)C2] | (S)-4-(4-(1,4-diazepan-1-yl)- 2-((1-methylpyrrolidin-2- yl)methoxy)-5,8-dihydro- pyrido[3,4-d]pyrimidin- 7(6H)-yl)naphthalen-2-ol | 489.55 |
| 699 | 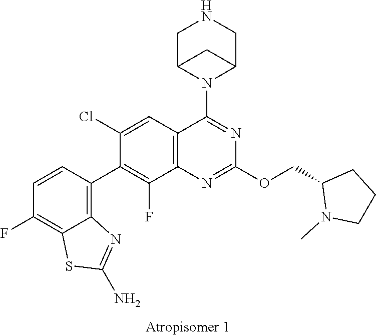 [SMILES: CN1CCC[C@H]1COc1nc(N2C3CNCC2C3)c2cc(Cl)c(-c3ccc(F)c4sc(N)nc34)c(F)c2n1] | 4-(4-(3,6-diazabicyclo- [3.1.1]heptan-6-yl)-6-chloro- 8-fluoro-2-(((S)-1-methyl- pyrrolidin-2-yl)methoxy)- quinazolin-7-yl)-7-fluoro- benzo[d]thiazol-2-amine | 558.1 |
| 700 | 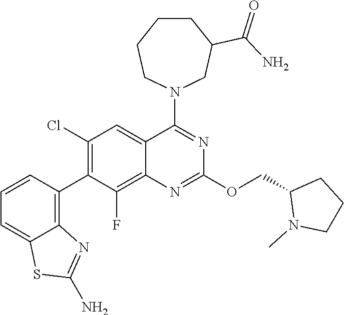 [SMILES: CN1CCC[C@H]1COc1nc(N2CCCCC(C(N)=O)C2)c2cc(Cl)c(-c3cccc4sc(N)nc34)c(F)c2n1] | 1-(7-(2-aminobenzo[d]- thiazol-4-yl)-6-chloro-8- fluoro-2-(((S)-1-methyl- pyrrolidin-2-yl)methoxy)- quinazolin-4-yl)azepane-3- carboxamide | 584.2 |
| 701 | 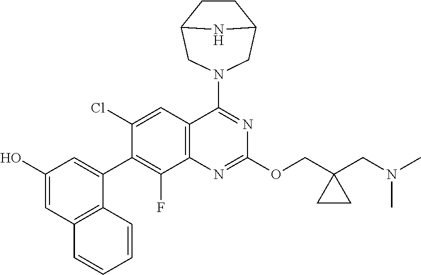 [SMILES: CN(C)CC1(COc2nc(N3CC4CCC(C3)N4)c3cc(Cl)c(-c4cc(O)cc5ccccc45)c(F)c3n2)CC1] | 4-(4-(3,8-diazabicyclo- [3.2.1]octan-3-yl)-6-chloro- 2-((1-((dimethylamino)- methyl)cyclopropyl)meth- oxy)-8-fluoroquinazolin-7- yl)naphthalen-2-ol | 562.5 |
| 702 | 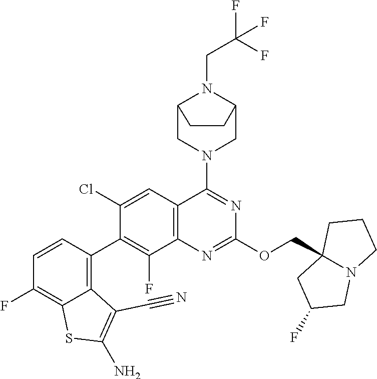 [SMILES: N#Cc1c(N)sc2c(F)ccc(-c3c(Cl)cc4c(N5CC6CCC(C5)N6CC(F)(F)F)nc(OC[C@@]56CCCN5C[C@H](F)C6)nc4c3F)c12] | 2-amino-4-(6-chloro-8- fluoro-2-(((2R,7aS)-2- fluorotetrahydro-1H-pyrrol- izin-7a(5H)-yl)methoxy)-4- ((1R,5S)-8-(2,2,2-trifluoro- ethyl)-3,8-diazabicyclo- [3.2.1]octan-3-yl)quin- azolin-7-yl)-7-fluorobenzo- [b]thiophene-3-carbonitrile | 722.2 |
| 703 | 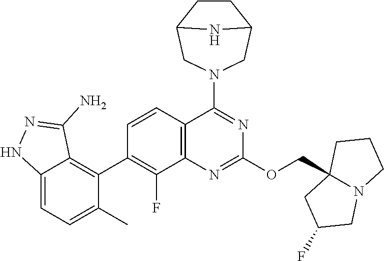 [SMILES: Cc1ccc2[nH]nc(N)c2c1-c1ccc2c(N3CC4CCC(C3)N4)nc(OC[C@@]34CCCN3C[C@H](F)C4)nc2c1F] | 4-(4-(3,8-diazabicyclo- [3.2.1]octan-3-yl)-8-fluoro- 2-(((2R,7aS)-2-fluorotetra- hydro-1H-pyrrolizin-7a(5H)- yl)methoxy)quinazolin-7- yl)-5-methyl-1H-indazol-3- amine | 561.5 |

### Example 2: Ras Sequence

```
Human K-Ras4b (SEQ ID NO. 1):
    1 MTEYKLVVVG AGGVGKSALT IQLIQNHFVD
        EYDPTIEDSY RKQVVIDGET
  51 CLLDILDTAG QEEYSAMRDQ YMRTGEGFLC
        VFAINNTKSF EDIHHYREQI
101 KRVKDSEDVP MVLVGNKCDL PSRTVDTKQA
        QDLARSYGIP FIETSAKTRQ
151 GVDDAFYTLV REIRKHKEKM SKDGKKKKKK
        SKTKCVIM
```

### Example 3: Protein Expression

DNA expression constructs encoding one or more protein sequences of interest (e.g., Kras fragments thereof, mutant variants thereof, etc.) and its corresponding DNA sequences are optimized for expression in E. coli and synthesized by, for example, the GeneArt Technology at Life Technologies. In some cases, the protein sequences of interest are fused with a tag (e.g., glutathione S-transferase (GST), histidine (His), or any other affinity tags) to facilitate recombinant expression and purification of the protein of interest. Such tag can be cleaved subsequent to purification. Alternatively, such tag may remain intact to the protein of interest and may not interfere with activities (e.g., target binding and/or phosphorylation) of the protein of interest

A resulting expression construct is additionally encoded with (i) att-site sequences at the 5′ and 3′ ends for subcloning into various destination vectors using, for example, the Gateway Technology, as well as (ii) a Tobacco Etch Virus (TEV) protease site for proteolytic cleavage of one or more tag sequences. The applied destination vectors can be a pET vector series from Novagen (e.g., with ampicillin resistance gene), which provides an N-terminal fusion of a GST-tag to the integrated gene of interest and/or a pET vector series (e.g., with ampicillin resistance gene), which provides a N-terminal fusion of a HIS-tag to the integrated gene. To generate the final expression vectors, the expression construct of the protein of interest is cloned into any of the applied destination vectors. The expression vectors are transformed into E. coli strain, e.g., BL21 (DE3). Cultivation of the transformed strains for expression is performed in 10 L and 1 L fermenter. The cultures are grown, for example, in Terrific Broth media (MP Biomedicals, Kat. #1 13045032) with 200 ug/mL ampicillin at a temperature of 37° C. to a density of 0.6 (OD600), shifted to a temperature of ˜27° C. (for K-Ras expression vectors) induced for expression with 100 mM IPTG, and further cultivated for 24 hours. After cultivation, the transformed E. coli cells are harvested by centrifugation and the resulting pellet is suspended in a lysis buffer, as provided below, and lysed by passing three-times through a high pressure device. The lysate is centrifuged (49000 g, 45 min, 4° C.) and the supernatant is used for further purification.

### Example 4: Ras Protein Purification

A Ras (e.g., K-Ras wildtype or a mutant such as K-Ras G12D, K-Ras G12V or K-RasG12C) construct or a variant thereof is tagged with GST. E. coli culture from a 10 L fermenter is lysed in lysis buffer (50 mM Tris HCl 7.5, 500 mM NaCl, 1 mM DTT, 0.5% CHAPS, Complete Protease Inhibitor Cocktail-(Roche)). As a first chromatography step, the centrifuged lysate is incubated with 50 mL Glutathione Agarose 4B (Macherey-Nagel; 745500.100) in a spinner flask (16 h, 100). The Glutathione Agarose 4B loaded with protein is transferred to a chromatography column connected to a chromatography system, e.g., an Akta chromatography system. The column is washed with wash buffer (50 mM Tris HCl 7.5, 500 mM NaCl, 1 mM DTT) and the bound protein is eluted with elution buffer (50 mM Tris HCl 7.5, 500 mM NaCl, 1 mM DTT, 15 mM Glutathione). The main fractions of the elution peak (monitored by OD280) is pooled. For further purification by size-exclusion chromatography, the above eluate volume is applied to a column Superdex 200 HR prep grade (GE Healthcare) and the resulting peak fractions of the eluted fusion protein is collected. Native mass spectrometry analyses of the final purified protein construct can be performed to assess its homogeneous load with GDP.

### Example 5: HTRF (Homogenous Time-Resolved Fluorescence Resonance Energy Transfer Assay

The ability of a compound of the present disclosure to inhibit a Ras protein signaling can be demonstrated by an HTRF assay. This assay can be also used to assess a selective inhibition of a mutant Ras protein relative to a wildtype, or relative to a different mutant Ras protein. For example, the equilibrium interaction of wildtype Kras or K-Ras mutant (e.g., wildtype or a mutant thereof including those mentioned in Example 6) with SOS1 (e.g., hSOS1) can be assessed as a proxy or an indication for a subject compound's ability to bind and inhibit Ras protein. HTRF assay detects from (i) a fluorescence resonance energy transfer (FRET) donor (e.g., antiGST-Europium) that is bound to GST-tagged K-Ras mutant to (ii) a FRET acceptor (e.g., anti-6His-XL665) bound to a His-tagged hSOS1.

The assay buffer can contain ˜5 mM HEPES pH 7.4, ˜150 mM NaCl, ˜ 1 mM DTT, 0.05% BSA and 0.0025% (v/v) Igepal. A Ras working solution is prepared in an assay buffer containing typically a suitable amount of the protein construct (e.g., GST-tagged K-Ras mutant) and the FRET donor (e.g., antiGST-Eu(K) from Cisbio, France). A SOS1 working solution is prepared in an assay buffer containing suitable amount of the protein construct (e.g., His-hSOS1) and the FRET acceptor (e.g., anti-6His-XL665 from Cisbio, France). A suitable amount of the protein construct will depend on the range of activity or range of IC50 values being detected or under investigation. For detecting IC50 within a range of 500 nM, the protein constructs of the same range of molarity can be utilized. An inhibitor control solution is prepared in an assay buffer containing comparable amount of the FRET acceptor without the SOS1 protein.

A fixed volume of DMSO with or without test compound is transferred into a 384-well plate. Ras working solution is added to all wells of the test plate. SOS1 working solution is added to all wells except for those that are subsequently filled the inhibitor control solution. Upon incubation for about 10 minutes or longer, the fluorescence is measured with a M1000Pro plate reader (Tecan) using HTRF detection (excitation 337 nm, emission 1: 620 nm, emission 2: 665 nm). Compounds are tested in duplicates at different concentrations (for example, 10 μM, 2.5 μM, 0.63 μM, 0.16 μM, 0.04 UM, 0.01 μM test compound). The ratiometric data (i.e., emission 2 divided by emission 1) is used to calculate IC50 values against Ras using GraphPad Prism (GraphPad software). Following this general procedure, samples were tested with or without a subject compound disclosed herein including compounds exemplified in Table 1 to assess their abilities to inhibit K-Ras signaling or their respective IC50 values against a given Ras protein (e.g., a given mutant K-Ras) of interest. IC50 data for certain compounds exemplified herein is presented in Table 2 below.

### Example 6: GTPase Activity Assay

The ability of any compound of the present disclosure to inhibit a Ras protein signalling can be demonstrated by a reduced GTPase activity. This assay can be also used to assess a selective inhibition of a mutant Ras protein relative to a wildtype, or relative to a different mutant Ras protein. For instance, the assay can be used to establish a subject compound's ability to selectively inhibit Kras G12D relative to wildtype, Kras G12V relative to wildtype, or KrasG12D relative KrasG12V. In particular, intrinsic and GTPase-activating protein (GAP)-stimulated GTPase activity for K-Ras construct or a mutant thereof can be measured using EnzCheck phosphate assay system (Life Technologies). For example K-Ras WT, K-Ras D154Q mutant, K-Ras G12D mutant, and K-Ras G12D/D154Q mutant proteins (2.5 mg/ml) in buffer (20 mmol/L Tris, pH 8.0, 50 mM NaCl) is loaded with GTP at room temperature for 2 hours by exposing to exchange buffer containing EDTA. Proteins are buffer exchanged to assay buffer (30 mM Tris, pH 7.5, 1 mM DTT) and the concentration is adjusted to 2 mg/ml. GTP loading is verified by back extraction of nucleotide using 6M urea and evaluation of nucleotide peaks by HPLC using an ion-exchange column. The assay is performed in a clear 384-well plate (Costar) by combining GTP-loaded K-Ras proteins (50 mM final) with 2-amino-6-mercapto-7-methylpurine ribonucleoside (MESG) (200 mM final), and purine nucleotide phosphorylase (5 U/ml final). GTP hydrolysis is initiated by the addition of MgCl₂ at a working concentration of 40 mM. For GAP stimulation, Ras p21 protein activator 1 (P120GAP) can be included at 50 mM. Absorbance at 360 nm can be measured every 8 to 15 s for 1,000 s at 20° C. Samples are tested with or without a subject compound disclosed herein including compounds exemplified in Table 1 to assess each compound's ability to inhibit signaling of a given Ras protein (e.g., a given mutant Kras) of interest.

### Example 7: Nucleotide Exchange Assay

The ability of a compound of the present disclosure to inhibit a Ras protein signaling can be demonstrated by a reduced nucleotide exchange activity. This assay can be also used to assess a selective inhibition of a mutant Ras protein relative to a wildtype, or relative to a different mutant Ras protein. For example, 250 nM or 500 nM GDP-loaded K-Ras proteins (e.g., wildtype or a mutant thereof including those mentioned in Example 6), each was incubated with different concentrations of compounds (for example ˜60 μM, ˜20 μM, ˜6.7 μM, ˜2.2 μM, ˜0.7 UM, ˜0.2 μM subject compound). A control reaction without subject compound was also included. SOS1 (catalytic domain) protein was added to the K-Ras protein solution. The nucleotide exchange reaction was initiated by adding fluorescent labelled GDP (Guanosine 5′-Diphosphate, BODIPY™ FL 2′-(or-3′)—O—(N-(2-Aminoethyl) Urethane) to a final concentration of 0.36 μM. Fluorescence was measured every 30 s for 70 minutes at 490 nm/515 nm (excitation/emission) in a M1000Pro plate reader (Tecan). Data is exported and analyzed to calculate an IC50 using GraphPad Prism (GraphPad Software). Sample(s) was tested with or without a subject compound disclosed herein including compound(s) exemplified in Table 1 to assess compound's ability to inhibit K-Ras signaling or its IC50 against a given Ras protein (e.g., a given mutant K-Ras) of interest. IC50 data for certain compounds exemplified herein against wildtype K-Ras or a mutant K-Ras (e.g., K-Ras G12D mutant) is presented in Table 2 below.

TABLE 2

| | IC50 against | IC50 against | | IC50 against mutant | IC50 against |
|---|---|---|---|---|---|
| Compound | mutant Kras | wildtype Kras | Compound | Kras | wildtype Kras |
| 101* | +++ | ++ | 102 | +++ | +++ |
| 103 | +++ | +++ | 104 | +++ | +++ |
| 105 | +++ | +++ | 106 | +++ | +++ |
| 107 | ++ | ++ | 108 | +++ | +++ |
| 109 | +++ | +++ | 110 | ++ | ++ |
| 111 | ++ | ++ | 112 | +++ | +++ |
| 113 | +++ | +++ | 114 | +++ | +++ |
| 115 | +++ | +++ | 116 | +++ | +++ |
| 117 | +++ | +++ | 118 | ++ | ++ |
| 119 | ++ | +++ | 120 | +++ | +++ |
| 121 | +++ | +++ | 122 | ++ | ++ |
| 123 | ++ | ++ | 124 | ++ | ++ |
| 125 | ++ | ++ | 126 | ++ | +++ |
| 127 | +++ | +++ | 128 | ++ | ++ |
| 129 | ++ | +++ | 130 | ++ | +++ |
| 131 | ++ | ++ | 132 | ++ | ++ |
| 133 | +++ | +++ | 134 | ++ | +++ |
| 135 | ++ | ++ | 136 | +++ | +++ |
| 137 | ++ | ++ | 138 | ++ | ++ |
| 139 | ++ | ++ | 140 | ++ | +++ |
| 141 | ++ | +++ | 142 | +++ | +++ |
| 143 | ++ | ++ | 144 | ++ | ++ |
| 145 | ++ | ++ | 146 | +++ | +++ |
| 147 | +++ | +++ | 148 | +++ | +++ |
| 149 | ++ | ++ | 150 | ++ | +++ |
| 151 | ++ | ++ | 152 | +++ | +++ |
| 153 | ++ | ++ | 154 | +++ | +++ |
| 155 | ++ | ++ | 156 | +++ | +++ |
| 157 | +++ | +++ | 158 | +++ | +++ |
| 159 | +++ | +++ | 160 | ++ | +++ |
| 161 | +++ | +++ | 162 | +++ | +++ |
| 163 | +++ | +++ | 164 | +++ | +++ |
| 165 | +++ | +++ | 166 | ++ | ++ |
| 167 | +++ | +++ | 168 | +++ | +++ |
| 169 | ++ | ++ | 170 | ++ | ++ |
| 171 | ++ | ++ | 172 | +++ | +++ |
| 173 | +++ | +++ | 174 | +++ | +++ |
| 175 | +++ | +++ | 176 | ++ | ++ |
| 177 | ++ | ++ | 178 | ++ | ++ |
| 179 | ++ | ++ | 180 | +++ | ++ |
| 181 | +++ | +++ | 182 | ++ | ++ |
| 183 | ++ | ++ | 184 | ++ | ++ |
| 185 | ++ | ++ | 186 | ++ | ++ |
| 187 | +++ | +++ | 188 | +++ | +++ |
| 189 | ++ | ++ | 190 | +++ | +++ |
| 195 | ++ | ++ | 196 | ++ | ++ |
| 201 | +++ | +++ | 202 | +++ | +++ |
| 203 | +++ | +++ | 204 | +++ | +++ |
| 205 | +++ | +++ | 206 | +++ | +++ |
| 207 | +++ | +++ | 208 | +++ | +++ |
| 209 | +++ | +++ | 210 | +++ | +++ |
| 211 | +++ | +++ | 212 | +++ | +++ |
| 213 | +++ | +++ | | | |
| 301 | +++ | +++ | 302 | +++ | +++ |
| 303 | ++ | +++ | 304 | +++ | +++ |
| 305 | +++ | +++ | 306 | +++ | +++ |
| 401 | ND | ND | 553 | +++ | ++ |
| 402 | ND | ND | 554 | ++ | ++ |
| 403 | +++ | +++ | 555 | +++ | ++ |
| 404 | ++ | +++ | 556 | +++ | ++ |
| 405 | ++ | +++ | 557 | +++ | ++ |
| 406 | +++ | +++ | 558 | ++ | ++ |
| 407 | +++ | +++ | 559 | ++ | ++ |
| 408 | ++ | +++ | 560 | ++ | ++ |
| 409 | ND | ND | 561 | ++ | ++ |
| 410 | ++ | +++ | 562 | +++ | ++ |
| 411 | ++ | +++ | 563 | ++ | ++ |
| 412 | ++ | +++ | 564 | ++ | ++ |
| 413 | +++ | +++ | 565 | +++ | ++ |
| 414 | ++ | +++ | 566 | +++ | ++ |
| 415 | ND | ND | 567 | ++ | ++ |
| 416 | ++ | +++ | 568 | +++ | ++ |
| 417 | ++ | +++ | 569 | +++ | ++ |
| 418 | ++ | +++ | 570 | +++ | ++ |
| 419 | +++ | +++ | 571 | ++ | ++ |
| 420 | ++ | +++ | 572 | ND | ND |
| 421 | ND | ND | 573 | +++ | ++ |
| 422 | ++ | +++ | 574 | +++ | ++ |
| 423 | ++ | +++ | 575 | +++ | ++ |
| 424 | ++ | +++ | 576 | +++ | ++ |
| 425 | ++ | +++ | 577 | ND | ND |
| 426 | ++ | +++ | 578 | +++ | ++ |
| 427 | +++ | +++ | 579 | ++ | ++ |
| 428 | ++ | +++ | 580 | ++ | ++ |
| 429 | ++ | +++ | 581 | +++ | ++ |
| 430 | ++ | +++ | 582 | ND | ND |
| 431 | +++ | +++ | 583 | +++ | ++ |
| 432 | ++ | +++ | 584 | ++ | ++ |
| 433 | +++ | +++ | 585 | ND | ND |
| 434 | +++ | +++ | 586 | +++ | ++ |
| 435 | +++ | +++ | 587 | ND | ND |
| 436 | ND | ND | 588 | +++ | ++ |
| 437 | ++ | +++ | 589 | ++ | ++ |
| 438 | ++ | +++ | 590 | ++ | ++ |
| 439 | +++ | +++ | 591 | +++ | ++ |
| 440 | ++ | +++ | 592 | +++ | ++ |
| 441 | ++ | +++ | 593 | +++ | ++ |
| 442 | +++ | +++ | 594 | +++ | ++ |
| 443 | +++ | +++ | 595 | +++ | ++ |
| 444 | ND | ND | 596 | ++ | ++ |
| 445 | ++ | +++ | 597 | ++ | ++ |
| 446 | ND | ND | 598 | +++ | ++ |
| 447 | ++ | +++ | 599 | ++ | ++ |
| 448 | +++ | +++ | 600 | +++ | ++ |
| 449 | ND | ND | 601 | +++ | ++ |
| 450 | ++ | +++ | 602 | ++ | ++ |
| 451 | ++ | +++ | 603 | ++ | ++ |
| 452 | +++ | +++ | 604 | +++ | ++ |
| 453 | ++ | +++ | 605 | +++ | ++ |
| 454 | +++ | +++ | 606 | ++ | ++ |
| 455 | +++ | +++ | 607 | +++ | ++ |
| 456 | ND | ND | 608 | ++ | ++ |
| 457 | ++ | +++ | 609 | +++ | ++ |
| 458 | ++ | +++ | 610 | ++ | ++ |
| 459 | +++ | +++ | 611 | ++ | ++ |
| 460 | +++ | +++ | 612 | ++ | ++ |
| 461 | ++ | +++ | 613 | ND | ND |
| 462 | +++ | +++ | 614 | ++ | ++ |
| 463 | +++ | +++ | 615 | +++ | ++ |
| 464 | +++ | +++ | 616 | ND | ND |
| 465 | +++ | +++ | 617 | +++ | ++ |
| 466 | +++ | +++ | 618 | ++ | ++ |
| 467 | ++ | +++ | 619 | ++ | ++ |
| 468 | +++ | +++ | 620 | ND | ND |
| 469 | ++ | +++ | 621 | +++ | ++ |
| 470 | +++ | +++ | 622 | ND | ND |
| 471 | ND | ND | 623 | +++ | ++ |
| 472 | +++ | +++ | 624 | ND | ND |
| 473 | +++ | +++ | 625 | ND | ND |
| 474 | +++ | +++ | 626 | ND | ND |
| 475 | +++ | +++ | 627 | ++ | ++ |
| 476 | +++ | +++ | 628 | ND | ND |
| 477 | +++ | +++ | 629 | ++ | ++ |
| 478 | ++ | +++ | 630 | ND | ND |
| 479 | ++ | +++ | 631 | ND | ND |
| 480 | +++ | +++ | 632 | +++ | ++ |
| 481 | +++ | +++ | 633 | +++ | ++ |
| 482 | ++ | +++ | 634 | ++ | ++ |
| 483 | ++ | +++ | 635 | ++ | ++ |
| 484 | ++ | +++ | 636 | ++ | ++ |
| 485 | +++ | +++ | 637 | ++ | ++ |
| 486 | ND | ND | 638 | +++ | ++ |
| 487 | ++ | +++ | 639 | +++ | ++ |
| 488 | ND | ND | 640 | +++ | ++ |
| 489 | +++ | +++ | 641 | ++ | ++ |
| 490 | ++ | +++ | 642 | +++ | ++ |
| 491 | ND | ND | 643 | +++ | ++ |
| 492 | ND | ND | 644 | +++ | ++ |
| 493 | ++ | +++ | 645 | ++ | ++ |
| 494 | +++ | +++ | 646 | ++ | ++ |
| 495 | ++ | +++ | 647 | ++ | ++ |
| 496 | +++ | +++ | 648 | ++ | ++ |
| 497 | ND | ND | 649 | +++ | ++ |
| 498 | ++ | +++ | 650 | ++ | ++ |
| 499 | +++ | +++ | 651 | +++ | ++ |
| 500 | +++ | +++ | 652 | ++ | ++ |
| 501 | +++ | ++ | 653 | +++ | ++ |
| 502 | ++ | ++ | 654 | ++ | ++ |
| 503 | ++ | ++ | 655 | ++ | ++ |
| 504 | +++ | ++ | 656 | +++ | ++ |
| 505 | ++ | ++ | 657 | ++ | ++ |
| 506 | +++ | ++ | 658 | ++ | ++ |
| 507 | +++ | ++ | 659 | ND | ND |
| 508 | +++ | ++ | 660 | ND | ND |
| 509 | ++ | ++ | 661 | ++ | ++ |
| 510 | ++ | ++ | 662 | ND | ND |
| 511 | +++ | ++ | 663 | ND | ND |
| 512 | +++ | ++ | 664 | ND | ND |
| 513 | +++ | ++ | 665 | ++ | ++ |
| 514 | ND | ND | 666 | ++ | ++ |
| 515 | ND | ND | 667 | +++ | ++ |
| 516 | ++ | ++ | 668 | ND | ND |
| 517 | ND | ND | 669 | +++ | ++ |
| 518 | +++ | ++ | 670 | ++ | ++ |
| 519 | ND | ND | 671 | ++ | ++ |
| 520 | ND | ND | 672 | +++ | ++ |
| 521 | ++ | ++ | 673 | +++ | ++ |
| 522 | +++ | ++ | 674 | +++ | ++ |
| 523 | +++ | ++ | 675 | ++ | ++ |
| 524 | +++ | ++ | 676 | ND | ND |
| 525 | ++ | ++ | 677 | +++ | ++ |
| 526 | +++ | ++ | 678 | ++ | ++ |
| 527 | ++ | ++ | 679 | +++ | ++ |
| 528 | +++ | ++ | 680 | +++ | ++ |
| 529 | ++ | ++ | 681 | +++ | ++ |
| 530 | ND | ND | 682 | +++ | ++ |
| 531 | ++ | ++ | 683 | ++ | ++ |
| 532 | +++ | ++ | 684 | +++ | ++ |
| 533 | ++ | ++ | 685 | +++ | ++ |
| 534 | ++ | ++ | 686 | +++ | ++ |
| 535 | +++ | ++ | 687 | ++ | ++ |
| 536 | ND | ND | 688 | ND | ND |
| 537 | ++ | ++ | 689 | ++ | ++ |
| 538 | ++ | ++ | 690 | +++ | ++ |
| 539 | ND | ND | 691 | ++ | ++ |
| 540 | ++ | ++ | 692 | +++ | ++ |
| 541 | ++ | ++ | 693 | ND | ND |
| 542 | +++ | ++ | 694 | ++ | ++ |
| 543 | +++ | ++ | 695 | ++ | ++ |
| 544 | ++ | ++ | 696 | ND | ND |
| 545 | ++ | ++ | 697 | ++ | ++ |
| 546 | +++ | ++ | 698 | ND | ND |
| 547 | +++ | ++ | 699 | ND | ND |
| 548 | ++ | ++ | 700 | +++ | ++ |
| 549 | ++ | ++ | 701 | +++ | ++ |
| 550 | +++ | ++ | 702 | ND | ND |
| 551 | +++ | ++ | 703 | ++ | ++ |
| 552 | +++ | ++ | | | |

where for compound nos. 101-196, ‘+++’ means IC50 at or less than about 0.5 uM;
‘++’ means IC50 greater than 0.5 uM;
*means the IC50 was assessed in accordance to the general methodology of example 7, and the rest compound nos. from 101-196 were assessed in accordance to the general methodology of example 5;
where for compound nos. 201-213, ‘+++’ means IC50 at or less than about 1.5 uM that was assessed in accordance to the general methodology of example 5.
where for compound nos. 301-306, ‘+++’ means IC50 at or less than about 1.5 uM; “++” means IC50 above 1.5 uM;
where for compound nos. 401-703, ‘+++’ means IC50 at or less than about 0.5 uM; “++” means IC50 above 0.5 uM.

### Example 8: Ras Cellular Assay

The ability of any compound of the present disclosure to inhibit a Ras protein signalling can be demonstrated by inhibiting growth of a given Kras mutant cells. For example, this assay can be also used to assess a selective growth inhibition of a mutant Ras protein relative to a wildtype, or relative to a different mutant Ras protein.

a. Growth of Cells with K-Ras G12C Mutation

MIA PaCa-2 (ATCC CRL-1420) and NCI-H1792 (ATCC CRL-5895) cell lines comprise a G12C mutation and can be used to assess Ras cellular signaling in vitro, e.g., in response to a subject inhibitor compounds of the present disclosure. This cellular assay can also be used to discern selective inhibition of a subject compounds against certain types of Kras mutants, e.g., more potent inhibition against KrasG12D relative to KrasG12C mutant, by using MIA PaCa-2 (G12C driven tumor cell line) as a comparison. MIA PaCa-2 culture medium is prepared with DMEM/Ham's F12 (e.g., with stable Glutamine, 10% FCS, and 2.5% Horse Serum. NCI-H1792 culture medium is prepared with RPMI 1640 (e.g., with stable Glutamine) and 10% FCS.

On a first day (e.g., Day 1), Softagar (Select Agar, Invitrogen, 3% in ddH20 autoclaved) is boiled and tempered at 48° C. Appropriate culture medium (i.e., medium) is tempered to 37° C. Agar (3%) is diluted 1:5 in medium (═0.6%) and 50 ml/well plated into 96 well plates (Corning, #3904), then incubated at room temperature for agar solidification. A 3% agar is diluted to 0.25% in medium (1:12 dilution) and tempered at 42° C. Cells are trypsinized, counted, and tempered at 37° C. The cells (e.g., MIA PaCa-2 at about 125-150 cells, NCI-H1792 at about 1000 cells) are resuspended in 100 mL 0.25% Agar and plated, followed by incubation at room temperature for agar solidification. The wells are overlaid with 50 mL of the medium. Sister wells in a separate plate are plated for time zero determination. All plates are incubated overnight at 37° C. and 5% CO₂.

On a second day (e.g., Day 2), time zero values are measured. A 40 mL volume of Cell Titer 96 Aqueous Solution (Promega) is added to each well and incubated in the dark at 37° C. and 5% CO₂. Absorption can be measured at 490 nm and reference wavelength 660 nm. DMSO-prediluted test compounds are added to wells of interest, e.g., with HP Dispenser, to one or more desired concentrations (e.g., a final DMSO concentration of 0.3%).

On a tenth day (e.g., Day 10), absorption by wells treated with the test compounds and control wells are measured with, for example, Cell Titer 96 AQueous and analyzed in comparison to the time zero measurements. The IC50 values are determined using the four parameter fit. The resulting IC50 value is a measurement of the ability of the compounds herein to reduce cell growth of Ras-driven cells (e.g., tumor cell lines) in vitro and/or in vivo.

b. Growth of Cells with K-Ras G12D Mutation

ASPC-1 (ATCC CRL-1682), Panc-10.05 (ATCC CRL-2547), A427 cell lines comprise a G12D mutation and can be used to assess Ras cellular signaling in vitro, e.g., in response to the compounds herein. ASPC-1 culture medium is prepared with RPMI-1640 and 10% heat-inactivated FBS. Panc-10.05 culture medium is prepared with RPMI-1640, 10 Units/ml human recombinant insulin, and 10% FBS. A427 cell culture is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384UZ). The day after plating, cells are treated with a dilution series (e.g., a 9 point 3-fold dilution series) of the compounds herein (e.g., approximately 40 μl final volume per well). Cell viability can be monitored (e.g., approximately 5 days later) according to the manufacturer's recommended instructions, where the CellTiter-Glo reagent is added (e.g., approximately 10 μl), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the compounds herein to reduce cell growth of Ras-driven cells (e.g., tumor cell lines) in vitro and/or in vivo. The IC50 values are determined using the four parameter fit. The resulting IC50 value is a measurement of the ability of the compounds herein to reduce cell growth of Ras-driven cells (e.g., tumor cell lines) in vitro and/or in vivo. The ability of one or more compounds exemplified in Table 1 including Compound A and Compound B to inhibit a Ras protein signalling is demonsrated by inhibiting growth of multiple cell lines (e.g., listed in Table 3 below) that comprise a given Kras mutation.

TABLE 3
IC50 values of Compounds A and B against various cancer cell lines

| | | | Cellular IC50 of | Cellular IC50 of |
|---|---|---|---|---|
| Cancer Cell Line | Tumor location | Disease | Compound A | Compound B |
| A375 | Skin | Skin Cancer | ++ | ++ |
| A427 | Lung | Lung cancer | +++ | ++ |
| AsPC-1 | Pancreas | Pancreatic Cancer | +++ | +++ |
| CORL23 | Lung | Lung cancer | | ++ |
| GP2D | Colon | Colon cancer | +++ | +++ |
| NCI-H358 | Lung | Lung cancer | | +++ |
| HPAC | Pancreas | Pancreatic Cancer | +++ | +++ |
| LCLC97TM1 | Lung | Lung cancer | | +++ |
| LS123 | Colon | Colon cancer | ++ | +++ |
| Ls513 | Colon | Colon cancer | +++ | ++ |
| MiaPaca2 | Pancreas | Pancreatic Cancer | | +++ |
| NCI-H441 | Lung | Lung cancer | | +++ |
| NCI-H727 | Lung | Lung cancer | | +++ |
| PA-TU-8902 | Pancreas | Pancreatic Cancer | | +++ |
| Panc 04.03 | Pancreas | Pancreatic Cancer | +++ | +++ |
| Panc-1 | Pancreas | Pancreatic Cancer | | +++ |
| SK-CO-1 | Colon | Colon cancer | | +++ |
| SW620 | Colon | Colon cancer | | +++ |
| SW626 | Colon | Colon cancer | | ++ |
| SW900 | Lung | Lung cancer | | ++ |

“++” in this Table 3 indicates IC50 is great than 0.5 uM;
“+++” in this Table 3 indicates IC50 is less than 0.5 uM

### Example 9: In Vivo Ras Inhibition

The in vivo reduction in Ras signaling output by a compound of the present disclosure is determined in a mouse tumor xenograft model.

a. Xenograft with K-Ras G12C Mutation

In an example, tumor xenografts are established by administration of tumor cells with K-Ras G12C mutation (e.g., MIA PaCa-2 cells) into mice, e.g., injection of the tumor cells into the right flanks of female BomTacNMRI-Foxn1nu mice with an age between 6 to 8 weeks.

In case of the subcutaneous (s.c.) MIA PaCa-2 xenograft mouse models, MIA PaCa-2 cells are grown in cell culture flasks in appropriate medium. Cultures are incubated at 37° ° C. and 5% CO₂ in a humidified atmosphere, with medium change or subcultivation performed 2-3 times a week. For injection, the cultured tumor cells are mixed with PBS including 5% FCS and Matrigel in a 1:1 ratio. About 1×10E7 cells in a volume of 100 μL is injected s.c. in each mouse to establish tumors. Mice are randomized into treatment groups of 7-10 mice, once tumors reach a desirable size (e.g., between about 86 to about 170 mm3, or between about 115 to about 170 mm³). Treatment with a subject compound disclosed herein or controls (e.g., vehicle control) may start on the day of randomization and can be continued until end of the study (e.g., 22 days). The test samples are administered intragastrically using a gavage needle at an application volume of 10 mL/kg in a volume of 10 mL/kg per mouse daily twice with a 6 h difference. In some cases, the test compounds are dissolved in 0.5% DMSO (or 0.5% and 0.5% Natrosol) in sterile PBS.

Mice are housed under standardized conditions at 21.5±1.5° C. and 55±10% humidity. Standardized irradiated diet and autoclaved tap water is provided ad libitum. In some cases, tags (e.g., ear tags, microchips implanted subcutaneously under isoflurane anesthesia) are used to identify each mouse. The tumor diameter is measured two or three times a week with a caliper. The volume of each tumor (in mm3) is calculated according to the formula “tumor volume=(π*length*width2)/6.” To monitor side effects of treatment, mice are inspected daily for abnormalities and body weight is determined, e.g., daily. Animals are sacrificed at the end of the study. Animals with necrotic tumors or tumor sizes exceeding 1500 mm³ are sacrificed early during the study for ethical reasons.

b. Xenograft with K-Ras G12D Mutation

In another example, tumor xenografts are established by administration of tumor cells with K-Ras G12D mutation (e.g., ASPC-1, Panc-10.05 cells, or Panc 04.03) into mice.

Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μl). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w2)/2 in which L and w refer to the length and width tumor diameter, respectively. Percent tumor volume change can be calculated using the following formula: (V_{final}−Vᵢₙᵢₜᵢₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜᵢₐₗ of treatment group)/(average V_{final}−Vᵢₙᵢₜᵢₐₗ of control group). When tumors reach a threshold average size (e.g., approximately 200-400 mm³). mice are randomized into 3-10 mice per group and are treated with vehicle (e.g., 100% Labrasol®) or a subject compound disclosed herein using, for example, a daily schedule by oral gavage. Results can be expressed as mean and standard deviation of the mean.

1-108. (canceled)

109. A compound, or a pharmaceutically acceptable salt or solvate thereof, having the formula: 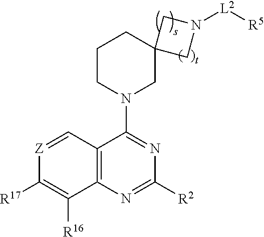
wherein:
Z is N or C(R⁸);
L¹ is selected from a bond, C₁-C₆alkyl, and —C(O)—;
R² is —OR¹²;
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)—N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is selected from C₃₋₆cycloalkyl, C₂₋₉heterocycloalkyl, C₆₋₁₀aryl, and C₁₋₉heteroaryl, wherein C₃₋₆cycloalkyl, C₂₋₉heterocycloalkyl, Co-10aryl, and C₁₋₉heteroaryl are optionally substituted with one, two, or three R^{20a};
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, Co-10aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, Co-10aryl, and C₁₋₉heteroaryl are optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl: or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₆cycloalkyl, C₂ heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{30g};
R¹⁷ is -L¹-R¹⁹:
L¹ is a bond:
R¹⁹ is selected from C₆₋₁₀aryl and C₁₋₉heteroaryl, wherein C₆₋₁₀aryl and C₁₋₉heteroaryl are optionally substituted with one, two, or three R²⁰ⁱ;
each R^{20a}, R^{20c}, R^{20d}, R^{20e}, R^{20f}, R^{20g}, and R²⁰ⁱ is independently selected from halogen, —CN, C₁₋₆alkyl, C₂₋₉alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
p is 1, 2, 3, 4, or 5;
s is 0 or 1;
tis 1 or 2; and
wherein s+t ≥2.

110. The compound of claim 109, or a pharmaceutically acceptable salt or solvate thereof, wherein Z is C(R⁸).

111. The compound of claim 109, or a pharmaceutically acceptable salt or solvate thereof, wherein Z is N.

112. The compound of claim 109, or a pharmaceutically acceptable salt or solvate thereof, wherein R² is selected from

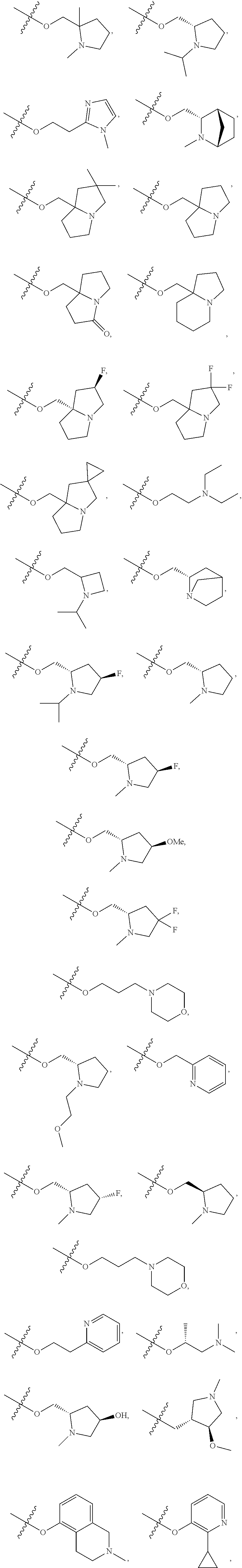

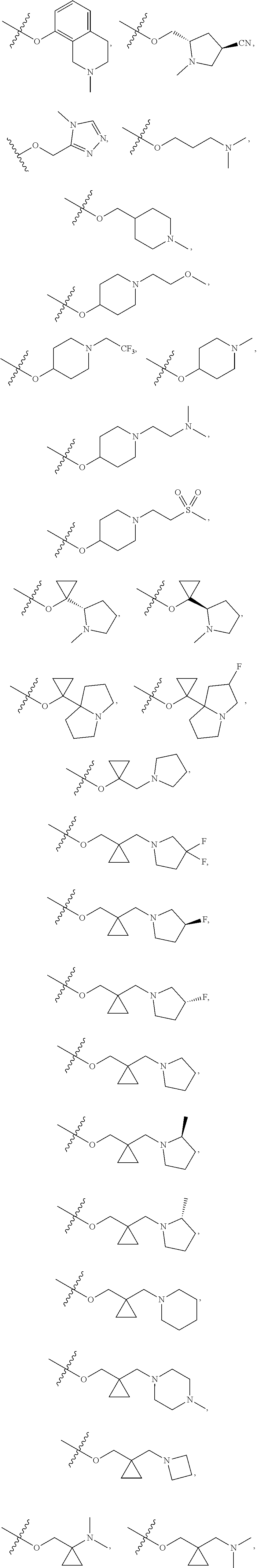

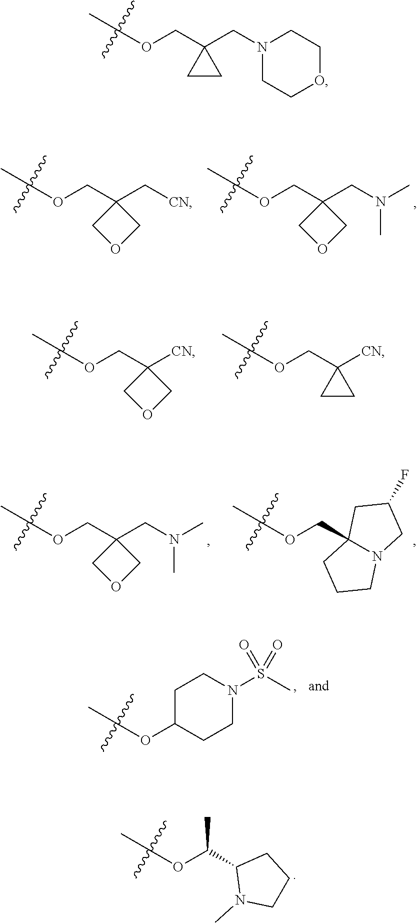

113. The compound of claim 109, or a pharmaceutically acceptable salt or solvate thereof, wherein R¹⁹ is C₁₋₉heteroaryl optionally substituted with one, two, or three R²⁰ⁱ.

114. The compound of claim 109, or a pharmaceutically acceptable salt or solvate thereof, wherein R¹⁹ is C₆₋₁₀aryl optionally substituted with one, two, or three R²⁰ⁱ.

115. The compound of claim 109, or a pharmaceutically acceptable salt or solvate thereof, wherein R¹⁹ is 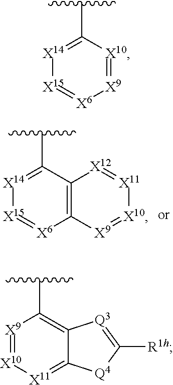
Q³ is C(R¹⁴);
Q⁴ is S;
X¹⁴, X¹⁵, X⁶, X⁹, X¹⁰, X¹¹, and X¹² are independently selected from C(R^{1a}) and N;
each R^{1a} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, —OH, and —NH₂;
R^{1d} is —CN; and
R^{1h} is —NH₂.

116. The compound of claim 109, or a pharmaceutically acceptable salt or solvate thereof, wherein R¹⁹ is selected from:

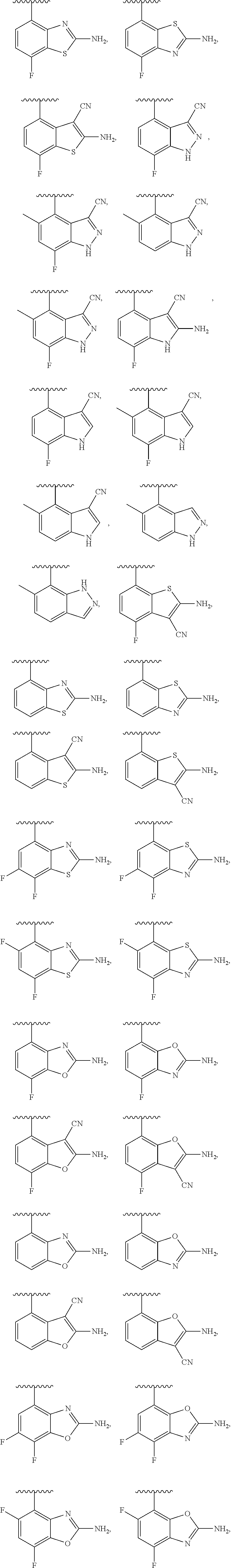

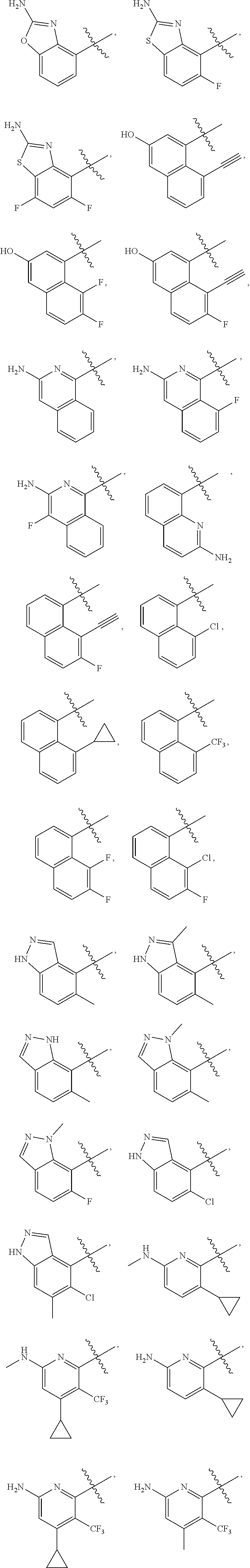

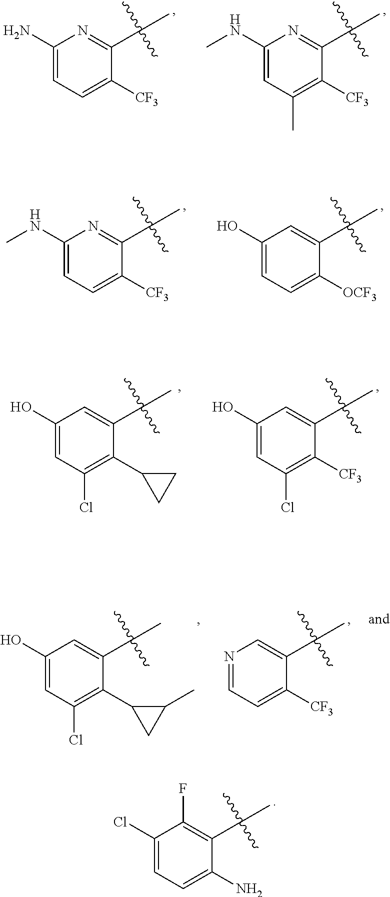

117. The compound of claim 109, or a pharmaceutically acceptable salt or solvate thereof, wherein L¹ is —C(O)—.

118. A compound, or a pharmaceutically acceptable salt or solvate thereof, having the formula: 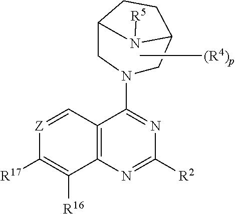
wherein:
Z is N or C(R⁸);
R² is selected from 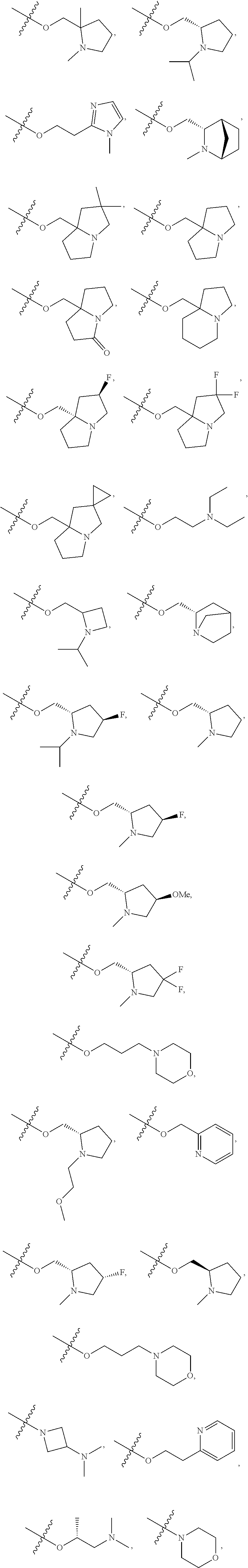 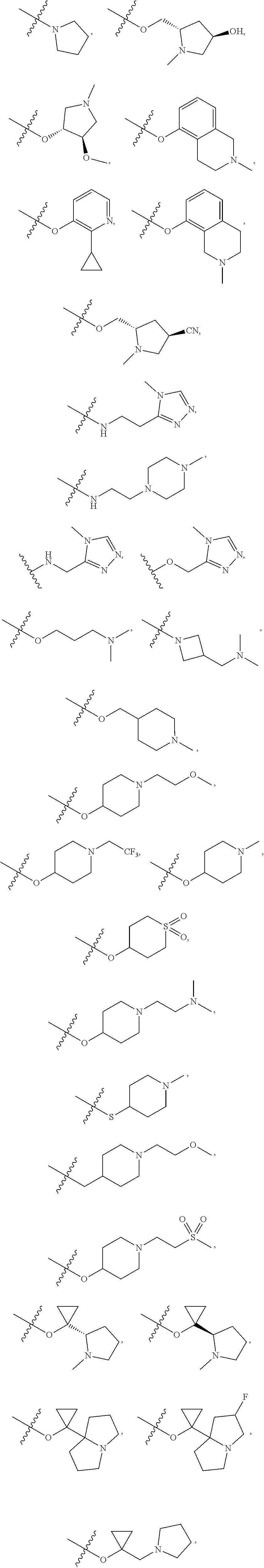 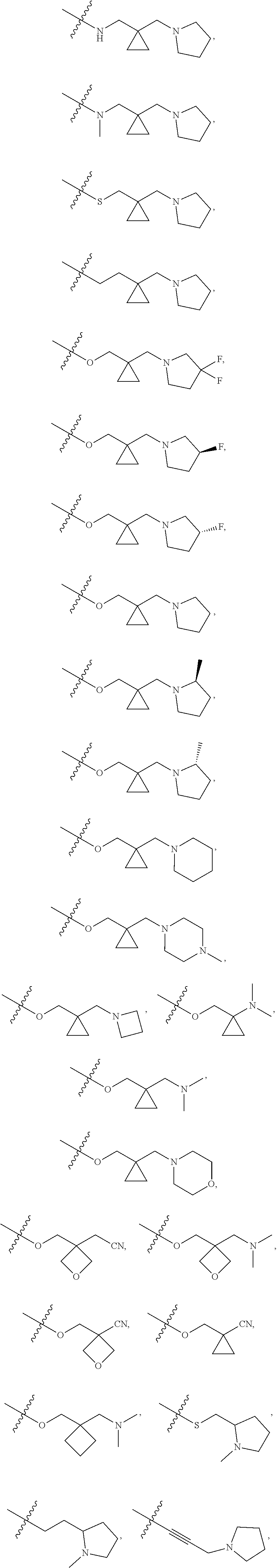 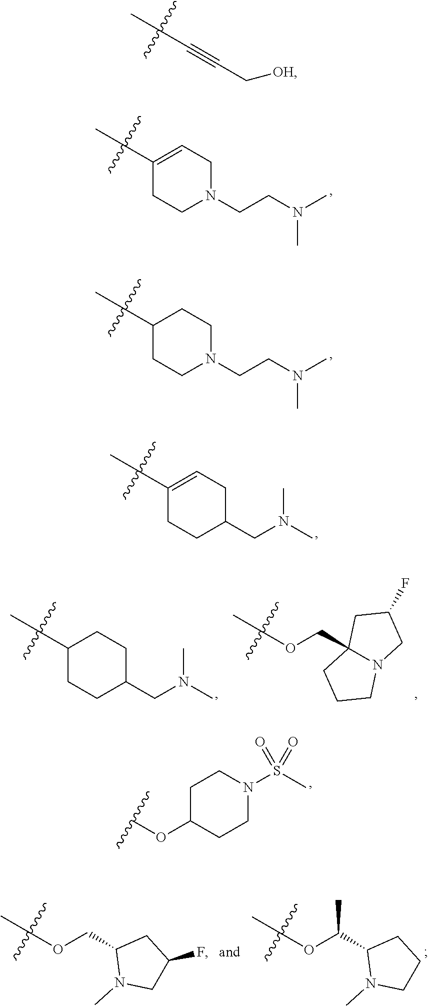
each R⁴ is independently selected from hydrogen, halogen, oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R⁵ is an electrophilic moiety capable of forming a covalent bond with a residue at position 12 of a KRAS protein: provided R⁵ is not selected from the group consisting of: 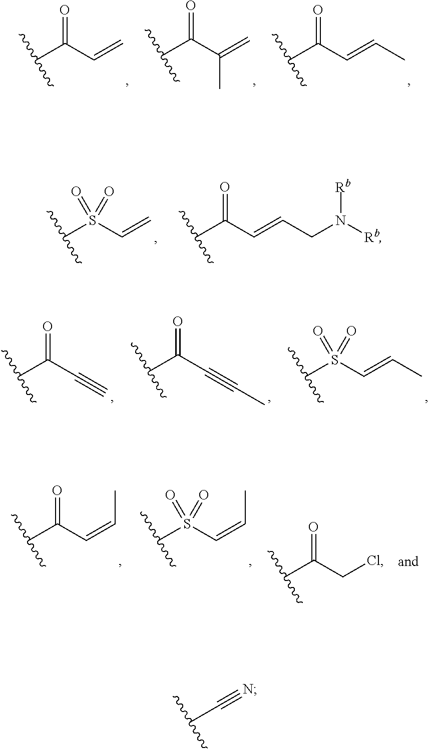
where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl;
R⁸ is hydrogen, halogen, C₁₋₆alkyl, or —OR¹², wherein C₁₋₆alkyl is optionally substituted with one, two, or three R^{20c};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₆cycloalkyl, C_{2 9}heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁰ⁱ;
R¹⁶ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)—N(R¹²)(R¹³), wherein C₁₋₆alkyl, C_{2 6}alkenyl, C₂₋₆alkynyl, C₃₋₆cycloalkyl, C₂ heterocycloalkyl, C₆₋₁₀aryl, and C₁₋₉heteroaryl are optionally substituted with one, two, or three R^{20g};
R¹⁷ is -L¹-R¹⁹;
L¹ is a bond:
R¹⁹ is selected from C₆₋₁₀aryl and C₁₋₉heteroaryl, wherein C₆₋₁₀aryl and C₁₋₉heteroaryl are optionally substituted with one, two, or three R²⁰ⁱ;
each R^{20a}, R^{20c}, R^{20d}, R^{20e}, R^{20f}, R^{20b}, and R²⁰ⁱ is independently selected from halogen, —CN, C₁₋₆alkyl, C₂₋₉alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR³¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl:
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl:
each R²⁴ is independently selected from H and C₁₋₆alkyl;
each R²⁵ is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; and
p is 1, 2, 3, 4, or 5.

119. The compound of claim 118, or a pharmaceutically acceptable salt or solvate thereof, wherein Z is C(R⁸).

120. The compound of claim 119, or a pharmaceutically acceptable salt or solvate thereof, wherein R⁸ is halogen, C₁₋₆alkyl, or —OR¹², wherein C₁₋₆alkyl is optionally substituted with one, two, or three R^{20c}.

121. The compound of claim 118, or a pharmaceutically acceptable salt or solvate thereof, wherein Z is N.

122. The compound of claim 118, or a pharmaceutically acceptable salt or solvate thereof, wherein R¹⁹ is 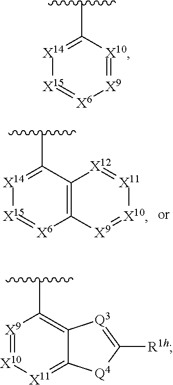
Q³ is C(R¹⁴);
Q⁴ is S;
X¹⁴, X¹⁵, X⁶, X⁹, X¹⁰, X¹¹, and X¹² are independently selected from C(R^{1a}) and N;
each R^{1a} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, —OH, and —NH₂;
R^{1d} is —CN; and
R^{1h} is —NH₂.

123. The compound of claim 118, or a pharmaceutically acceptable salt or solvate thereof, wherein R¹⁹ is selected from:

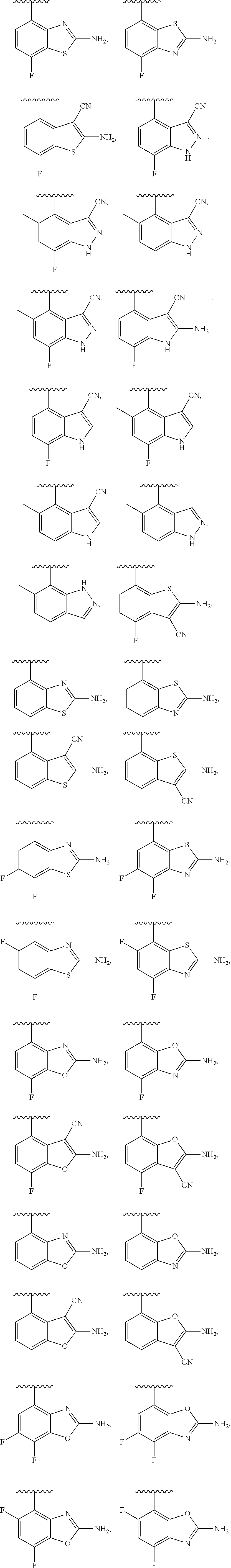

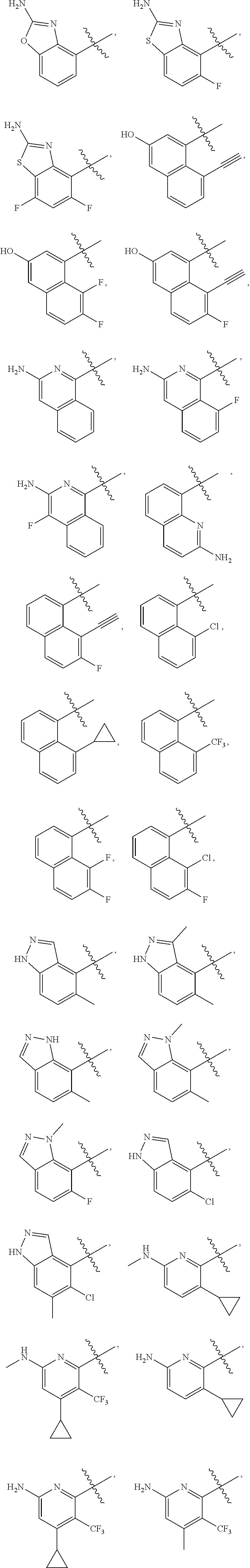

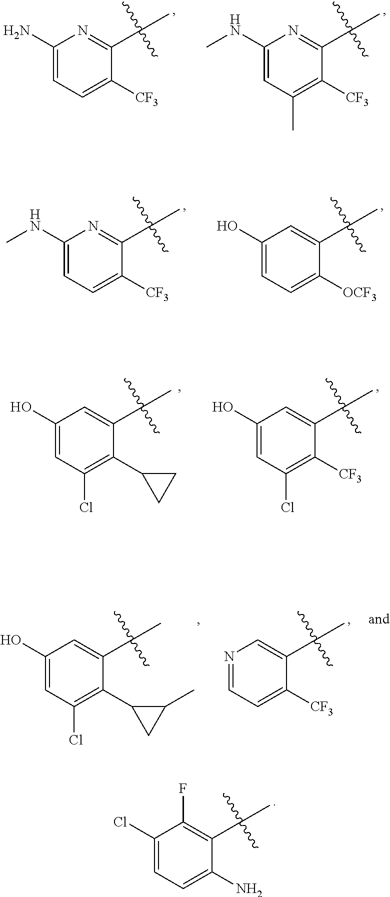

124. The compound of claim 118, or a pharmaceutically acceptable salt or solvate thereof, wherein R⁵ is —C(O)R¹⁵.

125. A pharmaceutical composition comprising a compound of claim 109, or a pharmaceutically acceptable salt or solvate thereof, and a pharmaceutically acceptable excipient.

126. A method of treating a Ras-driven cancer in a subject in need thereof, comprising administering to the subject a therapeutically effective amount of a compound of claim 109, or a pharmaceutically acceptable salt or solvate thereof.

127. A method of inhibiting activity of a Ras protein, comprising contacting a Ras protein with an effective amount of a compound of claim 109, or a pharmaceutically acceptable salt or solvate thereof, thereby modulating the activity of the Ras protein.

128. The method of claim 127, wherein the Ras protein is a G12D or G12S mutant K-Ras.
